(19) United States
(12) Patent Application Publication
(10) Pub. No.: US 2025/0214969 A1
(43) Pub. Date: Jul. 3, 2025

(54) PROTEIN DEGRADERS AND USES THEREOF

(71) Applicants: Kymera Therapeutics, Inc., Watertown, MA (US); KYMERA THERAPEUTICS, INC., Watertown, MA (US)

(72) Inventors: Bin YANG, Lexington, MA (US); Xiaozhang ZHENG, Lexington, MA (US); Michael D. SINTCHAK, Winchester, MA (US); Matthew M. WEISS, Boston, MA (US); Christopher M. YATES, Belmont, MA (US); Yi ZHANG, Belmont, MA (US); Xiao ZHU, Winchester, MA (US); Lewis Dale PENNINGTON, Watertown, MA (US); Nan JI, Watertown, MA (US)

(21) Appl. No.: 18/852,017

(22) PCT Filed: Mar. 31, 2023
(86) PCT No.: PCT/US2023/017071

§ 371 Date: Sep. 27, 2024

Related U.S. Application Data
(60) Provisional application No. 63/326,074, filed on Mar. 31, 2022.

(51) Int. Cl. C07D 401/14; A61K 31/4545; A61K 31/5517; A61P 35/00; C07D 213/74; C07D 409/12; C07D 487/04; C07D 495/14

(52) U.S. Cl. CPC C07D 401/14; A61K 31/4545; A61K 31/5517; A61P 35/00; C07D 213/74; C07D 409/12; C07D 487/04; C07D 495/14

(57) ABSTRACT

The present invention provides compounds, compositions thereof, and methods of using the same for the targeted degradation of proteins, and the treatment of target protein-mediated disorders.

### CROSS-REFERENCE TO RELATED APPLICATIONS

This application claims the benefit of priority to U.S. Provisional Appl. No. 63/326,074, filed Mar. 31, 2022, the entirety of which is herein incorporated by reference.

### TECHNICAL FIELD OF THE INVENTION

The present invention relates to compounds and methods useful for the modulation of targeted ubiquitination, especially with respect to a variety of polypeptides and other proteins, which are degraded and/or otherwise inhibited by compounds according to the present invention. The invention also provides pharmaceutically acceptable compositions comprising compounds of the present invention and methods of using said compositions in the treatment of various disorders.

### BACKGROUND OF THE INVENTION

Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases. These ligases comprise over 500 different proteins and are categorized into multiple classes defined by the structural element of their E3 functional activity.

Kelch domain-containing protein 2 (KLHDC2), also known as Help1, is a substrate-recognition component of a Cullin 2-RING (CRL2) E3 ubiquitin ligase complex of the DesCEND (destruction via C-end degrons) pathway, which recognizes a C-degron located at the extreme C terminus of target proteins, leading to their ubiquitination and degradation. The CRL2 (KLHDC2) complex specifically recognizes proteins with a diglycine (Gly-Gly) at the C-terminus, leading to their ubiquitination and degradation.

The UPP is used to induce selective protein degradation, including use of fusion proteins to artificially ubiquitinate target proteins and synthetic small-molecule probes to induce proteasome-dependent degradation. Bifunctional compounds composed of a target protein-binding ligand and an E3 ubiquitin ligase ligand induce proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful as biochemical reagents and lead to a new paradigm for the treatment of diseases by removing pathogenic or oncogenic proteins (Crews C, Chemistry & Biology, 2010, 17 (6): 551-555; Schnnekloth JS Jr., Chembiochem, 2005, 6 (1): 40-46).

An ongoing need exists in the art for effective treatments for disease, especially cancer. However, non-specific effects, and the inability to target and modulate certain classes of proteins altogether, such as transcription factors, remain as obstacles to the development of effective anti-cancer agents. As such, small molecule therapeutic agents that leverage or potentiate KLHDC2 substrate specificity and, at the same time, are “tunable” such that a wide range of protein classes can be targeted and modulated with specificity would be very useful as a therapeutic. Accordingly, there remains a need to find bifunctional compounds that utilize a KLHDC2 E3 ubiquitin ligase binding moiety in protein degraders useful as therapeutic agents.

### SUMMARY OF THE INVENTION

The present application relates novel compounds which modulate KLHDC2 and/or function to recruit targeted proteins to KLHDC2 for degradation, and methods of preparation and uses thereof. In particular, the present disclosure provides bifunctional compounds, which find utility as modulators of targeted ubiquitination of a variety of polypeptides and other proteins, which are then degraded and/or otherwise inhibited by the bifunctional compounds as described herein. An advantage of the compounds provided herein is that a broad range of pharmacological activities is possible, consistent with the degradation/inhibition of targeted polypeptides from virtually any protein class or family. In addition, the description provides methods of using an effective amount of the compounds as described herein for the treatment or amelioration of a disease condition, such as cancer.

The present application further relates to targeted degradation of proteins through the use of bifunctional molecules, including bifunctional molecules that link a KLHDC2 binding moiety to a ligand that binds the targeted protein. Such compounds have the general formula I:

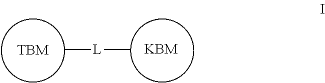

or a pharmaceutically acceptable salt thereof, wherein,
TBM is a target binding moiety capable of binding to a targeted protein(s);
L is a bivalent moiety that connects TBM to KBM; and
KBM is a ubiquitin binding moiety capable of binding to a KLHDC2 E3 ubiquitin ligase.

Compounds of the present invention, and pharmaceutically acceptable compositions thereof, are useful for treating a variety of diseases, disorders or conditions. Such diseases, disorders, or conditions include those described herein.

Compounds provided by this invention are also useful for the study of KLHDC2 and targeted proteins in biological and pathological phenomena; the study of KLHDC2 and targeted proteins occurring in bodily tissues; and the comparative evaluation of new KLHDC2 or targeted protein ligands or other regulators of KLHDC2 or targeted proteins in vitro or in vivo.

### BRIEF DESCRIPTION OF THE DRAWINGS

FIG. 1 shows STAT3 degradation of compounds I-482 to I-485 in HEK293 cells.

FIG. 2 shows BRD4 degradation of compounds I-478 to I-480 in HEK293 cells.

FIG. 3 shows BRD4 degradation of compound I-481 in HEK293 cells.

FIG. 4 shows KLHDC2-dependent BRD4 degradation of compound I-481 in HEK293 cells.

### DETAILED DESCRIPTION OF CERTAIN EMBODIMENTS

### 1. General Description of Certain Embodiments of the Invention:

Compounds of the present invention, and compositions thereof, are useful for the modulation KLHDC2 and targeted ubiquitination. As defined herein, the terms “binder,” “modulator,” and “ligand” are used interchangeably and describe a compound that binds to, modulates or is a ligand for KLHDC2 or a targeted protein.

In certain embodiments, the present invention provides a compound of formula I-a:

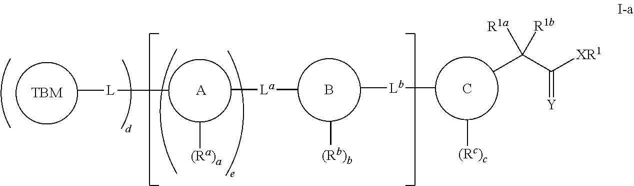

or a pharmaceutically acceptable salt thereof, wherein TBM and L are described and defined herein, and wherein:
R¹, R^{1a} and R^{1b} are each independently hydrogen or optionally substituted C₁₋₆ aliphatic;
each R^{a}, R^{b}, and R^{c} are each independently hydrogen, R^{A}, halogen, —CN, —NO₂, —OR, —SR, —NR₂, —S(O)₂R, —S(O)₂NR₂, —S(O)R, —S(O)(NR)R, —P(O)(OR)₂, —P(O)(NR₂)₂, —CFR₂, —CRF₂, —CF₃, —CR₂ (OR), —CR₂ (NR₂), —C(O)R, —C(O) OR, or —C(O)NR₂;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atom to form an optionally substituted 4-11 membered saturated or partially unsaturated carbocyclic or heterocyclic monocyclic, bicyclic, bridged bicyclic, spirocyclic, or heteroaryl ring having 0-3 heteroatoms, in addition to the atom to which they are attached, independently selected from nitrogen, oxygen, and sulfur;
Ring A is bivalent ring selected from phenylenyl, naphthylenyl, a 4-10 membered saturated or partially unsaturated monocyclic or bicyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur;
Ring B is bivalent ring selected from phenylenyl, a 3-10 membered saturated or partially unsaturated monocyclic, bicyclic, bridged bicyclic, or spirocyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur;
Ring C is bivalent ring selected from phenylenyl, a 4-10 membered saturated or partially unsaturated monocyclic or bicyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur;
each of L^{a} and L^{b} is independently a covalent bond or a C₁₋₃ bivalent straight or branched saturated or unsaturated hydrocarbon chain wherein 1-3 methylene units of the chain are independently and optionally replaced with —O—, —C(O)—, —C(S)—, —C(R)₂—, —CH(R)—, —CF(R)—, —C(F)₂—, —N(R)—, —S—, —S (O)₂— or —CR═CR—;
a, b, and c are each independently 0, 1, 2, 3 or 4;
each of e and d is independently 0 or 1;
X is —O—, —N(R)—, or —S—; and
Y is O, N (R), or S.

### 2. Compounds and Definitions:

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

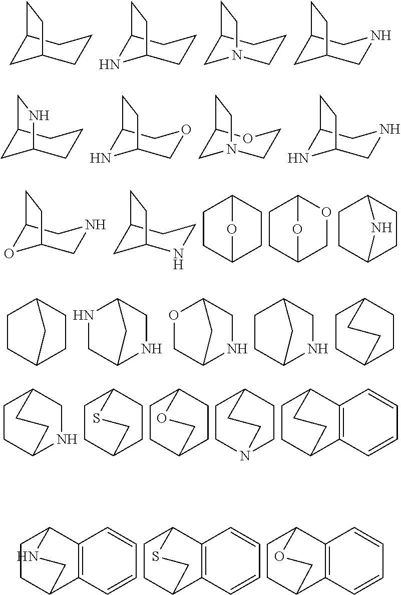

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

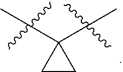 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 x electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 9-membered monocyclic or 7- to 11-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention arc preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O (CH₂)₀₋₄R^{o}, —O—(CH₂)₀₋₄C(O) OR^{o}; —) (CH₂)₀₋₄CH (OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —)(CH₂)₀₋₄N(R^{o}2; —)(CH₂)₀₋₄N (R^{o}C(O)R^{o}; —N(R^{o}C(S)R^{o}; —(CH₂)₀₋₄N (R^{o}C(O)NR^{o}2; —N(R^{o})C(S)NR^{o}₂; —(CH₂)₀₋₄N (R^{o}C(O) OR^{o}; —N(R^{o}N(R^{o}C(O)R^{o}; —N(R^{o}N (R^{o}C(O)NR^{o}2; —N(R^{o}N (R^{o}C(O) OR^{o}; —(CH₂)₀₋₄C(O)R^{o}; —C(S)R^{o}; —(CH₂)₀₋₄C(O)OR^{o}; —(CH₂)₀₋₄C(O)SR^{o}; (CH₂)₀₋₄C(O)OSiR^{o}₃; —(CH₂)₀₋₄OC(O)R^{o}; —OC(O)(CH₂)₀₋₄SR^{o}; —SC(S) SR^{o}; —(CH₂)₀₋₄SC(O)R^{o}; —(CH₂)₀₋₄C(O)NR^{o}₂; —C(S)NR^{o}₂; —C(S) SR^{o}; —(CH₂)₀₋₄₀C(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S (O)₂R^{o}; —(CH₂)₀₋₄S (O)₂OR^{o}; —(CH₂)₀₋₄OS (O)₂R^{o}; —S(O)₂NR^{o}₂; —(CH₂)₀₋₄S (O)R^{o}; —N(R^{o}S(O)₂NR^{o}₂; —)N(R^{o}S (O)₂R^{o}; —N(OR^{o})R^{o}; —C(NH)NR^{o}₂; —(CH₂)₀₋₄P(O)₂R^{o}; —(CH₂)₀₋₄P(O)R^{o}₂; —(CH₂)₀₋₄OP(O)R^{o}₂; —(CH₂)₀₋₄OP(O)(OR^{o})₂; —SiR^{o}₃; —(C₁₋₄ straight or branched)alkylene)O—N(R^{o}₂; or —(C₁₋₄ straight or branched)alkylene)C(O)O—N(R^{o}₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), arc independently halogen, —(CH₂)₀₋₂R^{o}, -(haloR^{•}), —(CH₂)₀₋₂OH, —(CH₂)₀₋₂OR^{•}, —(CH₂)₀₋₂CH (OR^{•})₂; —)O(haloR^{•}, —CN, —N₃, —(CH₂)₀₋₂C(O)R^{•}, —(CH₂)₀₋₂C(O) OH, —(CH₂)₀₋₂C(O) OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O) SR^{•}, —(C₁₋₄ straight or branched alkylene)C(O) OR^{•}, or —SSR^{•} wherein each R^{o} is unsubstituted or where preceded by “halo” is substituted only with one or more halogens, and is independently selected from C₁₋₄ aliphatic, —CH₂Ph, —O (CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{•} include ═O and ═S.

Suitable divalent substituents on a saturated carbon atom of an “optionally substituted” group include the following: ═O, ═S, ═NNR^{*}₂, ═NNHC(O)R^{*}, ═NNHC(O) OR^{*}, ═NNHS (O)₂R^{*}, ═NR^{*}, ═NOR^{*}, —O (C (R^{*}₂))₂₋₃₀—, or —S(C(R^{*}₂))₂₋₃S—, wherein each independent occurrence of R^{•}is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR^{*}₂)₂₋₃O—, wherein each independent occurrence of R^{•}is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R^{*} include halogen, —R^{•}, -(haloR^{•}, —OH, —OR^{•}, —O(haloR^{•}), —CN, —C(O)OH, —C(O)OR^{•}, —NH₂, —NHR^{•}, —NR^{•}₂, or —NO₂, wherein each R′ is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include-R^{†}, —NR^{†}₂, —C(O)R^{†}, C(O)OR^{†}, —C(O)C(O)R^{•}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{•}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}, —OH, —OR^{•}, —O(haloR^{•}, —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O (CH₂)₀₋₁Ph, or a 5-6-membered saturated, partially unsaturated, or aryl ring having 0-4 heteroatoms independently selected from nitrogen, oxygen, or sulfur.

As used herein, the term “provided compound” refers to any genus, subgenus, and/or species set forth herein.

As used herein, the term “pharmaceutically acceptable salt” refers to those salts which are, within the scope of sound medical judgment, suitable for use in contact with the tissucs of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In certain embodiments, a provided compound may be substituted with one or more deuterium atoms.

As used herein, the term “provided compound” refers to any genus, subgenus, and/or species set forth herein.

As used herein, the term “binder” or “inhibitor” is defined as a compound that binds to KLHDC2 and binds to or inhibits a targeted protein with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

As used herein, the term “degrader” is defined as a heterobifunctional compound that binds to and/or inhibits both a target protein and an E3 ligase with measurable affinity resulting in the ubiqitination and subsequent degradation of the target protein. In certain embodiments, a degrader has an DC50 of less than about 50 M, less than about 1 μM, less than about 500 nM, less than about 100 nM, less than about 10 nM, or less than about 1 nM.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41:2596-99 and Sun et al., Bioconjugate Chem., 2006, 17:52-57.

As used herein, the term “detectable moiety” is used interchangeably with the term “label” and relates to any moiety capable of being detected, e.g., primary labels and secondary labels. Primary labels, such as radioisotopes (e.g., tritium, 32P, 33P, 35S, or 14C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R⁶G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy] phenyl]-3-methylglyceronyl] isonipecotic Acid, 4′-[2,3,5,6-Tetrafluoro-4-(pentafluorophenoxyl)] methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably modulate,” as used herein, means a measurable change in a KLHDC2 activity between a sample comprising a compound of the present invention, or composition thereof, and KLHDC2, and an equivalent sample comprising KLHDC2, in the absence of said compound, or composition thereof.

### 3. Description of Exemplary Embodiments:

The compounds of the present application include KLHDC2 binding compounds and bifunctional molecules that link a KLHDC2 binding moiety to a ligand that bind target proteins, bifunctional compounds having the general formula I:

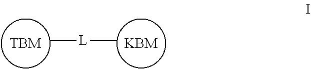

or a pharmaceutically acceptable salt thereof, wherein,
TBM is a target binding moiety capable of binding to a targeted protein(s);
L is a bivalent moiety that connects TBM to KBM; and
KBM is a E3 ubiquitin binding moiety capable of binding to a KLHDC2 E3 ubiquitin ligase.

### KLHDC2 Binding Moiety (KBM)

As described above and in certain embodiments, the present invention provides a compound of formula I-a:

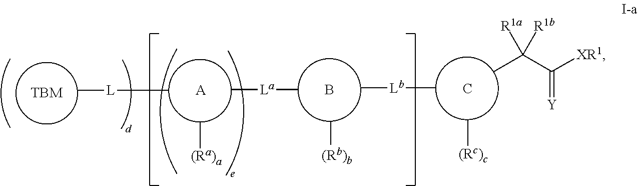

or a pharmaceutically acceptable salt thereof, wherein:
R¹, R^{1a} and R^{1b} are each independently hydrogen or optionally substituted C₁₋₆ aliphatic;
each R^{a}, R^{b}, and R^{c} are each independently hydrogen, R^{A}, halogen, —CN, —NO₂, oxo, —OR, —SR, —NR₂, —S(O)₂R, —S(O)₂NR₂, —S(O)R, —S(O)(NR)R, —P(O)(OR)₂, —P(O)(NR₂)₂, —CFR₂, —CRF₂, —CF₃, —CR₂ (OR), —CR₂ (NR₂), —C(O)R, —C(O) OR, or —C(O)NR₂;
each R^{A} is independently an optionally substituted group selected from C₁₋₁₀ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atom to form an optionally substituted 4-11 membered saturated or partially unsaturated carbocyclic or heterocyclic monocyclic, bicyclic, bridged bicyclic, spirocyclic, or heteroaryl ring having 0-3 heteroatoms, in addition to the atom to which they are attached, independently selected from nitrogen, oxygen, and sulfur;
Ring A is bivalent ring selected from phenylenyl, naphthylenyl, a 4-10 membered saturated or partially unsaturated monocyclic or bicyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur;
Ring B is bivalent ring selected from phenylenyl, a 3-10 membered saturated or partially unsaturated monocyclic, bicyclic, bridged bicyclic, or spirocyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur;
Ring C is bivalent ring selected from phenylenyl, a 4-10 membered saturated or partially unsaturated monocyclic or bicyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur; each of L^{a} and L^{b} is independently a covalent bond or a C₁₋₃ bivalent straight or branched saturated or unsaturated hydrocarbon chain wherein 1-3 methylene units of the chain are independently and optionally replaced with —O—, —C(O)—, —C(S)—, —C(R)₂—, —CH(R)—, —CF (R)—, —C(F)₂—, —N(R)—, —S—, —S(O)₂— or —CR═CR—;
a, b, and c are each independently 0, 1, 2, 3 or 4;
each of e and d is independently 0 or 1;
X is —O—, —N(R)—, or —S—;
Y is O, N (R), or S;
L is a covalent bond or a bivalent, saturated or unsaturated, straight or branched C₁₋₅₀ hydrocarbon chain, wherein 0-6 methylene units of L are independently replaced by —Cy—, —CRF—, —CF₂—, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N (R)C(O)O—,

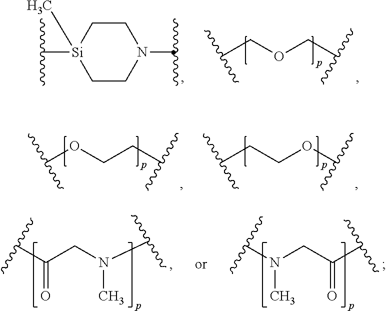

each —Cy— is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
each p is independently 0, 1, 2, 3, 4, 5, 6, 7, 8, 9, or 10; and
   TBM is a target binding moiety.

As defined above and described herein, R¹, R^{1a} and R^{1b} are each independently hydrogen or optionally substituted C₁₋₆ aliphatic.

In some embodiments, R¹ is hydrogen. In some embodiments, R¹ is an optionally substituted C₁₋₆ aliphatic. In some embodiments, R^{1a} is hydrogen. In some embodiments, R^{1a} is an optionally substituted C₁₋₆ aliphatic. In some embodiments, R^{1b} is hydrogen. In some embodiments, R^{1b} is an optionally substituted C₁₋₆ aliphatic.

In some embodiments, R¹, R^{1a} and R^{1b} are selected from those depicted in Table 1A and 1B, below.

As defined above and described herein, each R^{a}, R^{b}, and R^{c} are each independently hydrogen, R^{A}, halogen, —CN, —NO₂, —OR, oxo, —SR, —NR₂, —S(O)₂R, —S(0)₂NR₂, —S(O)R, —S(O)(NR)R, —P(O)(OR)₂, —P(O)(NR₂)₂, —CFR₂, —CRF₂, —CF₃, —CR₂(OR), —CR₂(NR₂), —C(O)R, —C(O) OR, or —C(O)NR₂.

In some embodiments, one or more of R^{a}, R^{b}, and R^{c} are hydrogen. In some embodiments, one or more of R^{a}, R^{b}, and R^{c} are R^{A}. In some embodiments, one or more of R^{a}, R^{b}, and R^{c} are halogen. In some embodiments, one or more of R^{a}, R^{b}, and R^{o} are —CN. In some embodiments, one or more of R^{a}, R^{b}, and R^{c} are —NO₂. In some embodiments, one or more of R^{a}, R^{b}, and R^{c} are —OR. In some embodiments, one or more of R^{a}, R^{b}, and R^{c} are oxo. In some embodiments, one or more of R^{a}, R^{b}, and R^{c} are —SR. In some embodiments, one or more of R^{a}, R^{b}, and R^{c} are —NR₂. In some embodiments, one or more of R^{a}, R^{b}, and R^{c} are —S(O)₂R. In some embodiments, one or more of R^{a}, R^{b}, and R^{c} are —S(O)₂NR₂. In some embodiments, one or more of R^{a}, R^{b}, and R^{c} are —S(O)R, —S(O)(NR)R. In some embodiments, one or more of R^{a}, R^{b}, and R^{o} are —P(O)(OR)₂. In some embodiments, one or more of R^{a}, R^{b}, and R^{o} are —P(O)(NR₂)₂. In some embodiments, one or more of R^{a}, R^{b}, and R^{c} are —CFR₂. In some embodiments, one or more of R^{a}, R^{b}, and R^{o} are —CRF₂. In some embodiments, one or more of R^{a}, R^{b}, and R^{c} are —CF₃. In some embodiments, one or more of R^{a}, R^{b}, and R^{c} are —CR₂(OR). In some embodiments, one or more of R^{a}, R^{b}, and R^{o} are —CR₂(NR₂). In some embodiments, one or more of R^{a}, R^{b}, and R^{o} are —C(O)R. In some embodiments, one or more of R^{a}, R^{b}, and R^{c} are —C(O)OR. In some embodiments, one or more of R^{a}, R^{b}, and R^{o} are —C(O)NR₂.

In some embodiments, R^{a}, R^{b}, and R^{c} are selected from those depicted in Table 1A and 1B, below.

As defined above and described herein, each R^{A} is independently an optionally substituted group selected from C₁₋₁₀ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, each R^{A} is independently an optionally substituted group selected from C₁₋₁₀ aliphatic. In some embodiments, each R^{A} is independently an optionally substituted phenyl. In some embodiments, each R^{A} is independently an optionally substituted 3-7 membered saturated or partially unsaturated carbocyclic or heterocyclic ring having 1-2 heteroatoms independently selected from nitrogen, oxygen, and sulfur. In some embodiments, each R^{A} is independently an optionally substituted 5-6 membered heteroaryl ring having 1-4 heteroatoms independently selected from nitrogen, oxygen, and sulfur.

In some embodiments, R^{A} is —(CH₂)₃NH₂. In some embodiments, R^{A} is —(CH₂)₃NHCO₂tBu. In some embodiments, R^{A} is —(CH₂)₆NH₂. In some embodiments, R^{A} is —(CH₂)₆NHCO₂tBu. In some embodiments, R^{A} is —(CH₂),NH₂. In some embodiments, R^{A} is —(CH₂),NHCO₂tBu. In some embodiments, R^{A} is —(CH₂)₂CO₂H. In some embodiments, R^{A} is —(CH₂)₅CO₂H. In some embodiments, R^{A} is —(CH₂)₆CO₂H. In some embodiments, R^{A} is —(CH₂)₃CO₂H.

In some embodiments, each R^{A} is selected from those depicted in Table 1, below.

As defined above and described herein, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom are optionally taken together with their intervening atom to form an optionally substituted 4-11 membered saturated or partially unsaturated carbocyclic or heterocyclic monocyclic, bicyclic, bridged bicyclic, spirocyclic, or heteroaryl ring having 0-3 heteroatoms, in addition to the atom to which they are attached, independently selected from nitrogen, oxygen, and sulfur.

In some embodiments, R is hydrogen. In some embodiments, R is an optionally substituted C₁-6 aliphatic. In some embodiments, R is an optionally substituted phenyl. In some embodiments, R is an optionally substituted 4-7 membered saturated or partially unsaturated heterocyclic having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur. In some embodiments, two R groups on the same atom are optionally taken together with their intervening atom to form an optionally substituted 4-11 membered saturated or partially unsaturated carbocyclic or heterocyclic monocyclic, bicyclic, bridged bicyclic, spirocyclic, or heteroaryl ring having 0-3 heteroatoms, in addition to the atom to which they are attached, independently selected from nitrogen, oxygen, and sulfur.

In some embodiments, R is selected from those depicted in Table 1A and 1B, below.

As defined above and described herein, Ring A is bivalent ring selected from phenylenyl, naphthylenyl, a 4-10 membered saturated or partially unsaturated monocyclic or bicyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur.

In some embodiments, Ring A is phenylenyl. In some embodiments, Ring A is naphthylenyl. In some embodiments, Ring A is a 4-10 membered saturated or partially unsaturated monocyclic or bicyclic carbocyclylenyl or heterocyclylenyl having 1-3 heteroatoms independently selected from nitrogen, oxygen, and sulfur. In some embodiments, Ring A is a 5-10 membered monocyclic or bicyclic heteroarylenyl having 1-4 heteroatoms independently selected from nitrogen, oxygen, and sulfur. In some embodiments, Ring A is 10-membered bicyclic heteroarylenyl having 1-4 heteroatoms independently selected from nitrogen, oxygen, and sulfur.

In some embodiments, Ring A is selected from those depicted in Table 1A and 1B, below.

As defined above and described herein, Ring B is bivalent ring selected from phenylenyl, a 3-10 membered saturated or partially unsaturated monocyclic, bicyclic, bridged bicyclic, or spirocyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur.

In some embodiments, Ring B is phenylenyl. In some embodiments, Ring B is a 3-10 membered saturated or partially unsaturated monocyclic, bicyclic, bridged bicyclic, or spirocyclic carbocyclylenyl or heterocyclylenyl having 1-3 heteroatoms independently selected from nitrogen, oxygen, and sulfur. In some embodiments, Ring B is a 5-10 membered monocyclic or bicyclic heteroarylenyl having 1-4 heteroatoms independently selected from nitrogen, oxygen, and sulfur.

In some embodiments, Ring B is selected from those depicted in Table 1A and 1B, below.

As defined above and described herein, Ring C is bivalent ring selected from phenylenyl, a 4-10 membered saturated or partially unsaturated monocyclic or bicyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur.

In some embodiments, Ring C is phenylenyl. In some embodiments, Ring C is a 4-10 membered saturated or partially unsaturated monocyclic or bicyclic carbocyclylenyl or heterocyclylenyl having 1-3 heteroatoms independently selected from nitrogen, oxygen, and sulfur. In some embodiments, Ring C is a 5-10 membered monocyclic or bicyclic heteroarylenyl having 1-4 heteroatoms independently selected from nitrogen, oxygen, and sulfur.

In some embodiments, Ring C is selected from those depicted in Table 1A and 1B, below.

As defined above and described herein, each of L^{a} and L^{b} are independently a covalent bond or a C₁₋₃ bivalent straight or branched saturated or unsaturated hydrocarbon chain wherein 1-3 methylene units of the chain are independently and optionally replaced with —O—, —C(O)—, —C(S)—, —C(R)₂—, —CH(R)—, —CF(R)—, —C(F)₂—, —N(R)—, —S—, —S(O)₂— or —CR═CR—.

In some embodiments, L^{a} is a covalent bond. In some embodiments, L^{a} is a C₁₋₃ bivalent straight or branched saturated or unsaturated hydrocarbon chain wherein 1-3 methylene units of the chain are independently and optionally replaced with —O—, —C(O)—, —C(S)—, —C(R)₂—, —CH(R)—, —CF(R)—, —C(F)₂—, —N(R)—, —S—, —S(O)₂— or —CR═CR—. In some embodiments, L^{a} is a covalent bond. In some embodiments, L^{b} is a C₁₋₃ bivalent straight or branched saturated or unsaturated hydrocarbon chain wherein 1-3 methylene units of the chain are independently and optionally replaced with —O—, —C(O)—, —C(S)—, —C(R)₂—, —CH(R)—, —CF(R)—, —C(F)₂—, —N(R)—, —S—, —S(O)₂— or —CR═CR—

In some embodiments, L^{a} and L′ are selected from those depicted in Table 1A and 1B, below.

As defined above and described herein, a, b, and c are each independently 0, 1, 2, 3 or 4.

In some embodiments, one or more of a, b, and c is 0. In some embodiments, one or more of a, b, and c is 1. In some embodiments, one or more of a, b, and c is 2. In some embodiments, one or more of a, b, and c is 3. In some embodiments, one or more of a, b, and c is 4.

In some embodiments, a, b, and c are selected from those depicted in Table 1A and 1B, below.

As defined above and described herein, d is 0 or 1.

In some embodiments, d is 0. In some embodiments, dis 1.

In some embodiments, d is selected from those depicted in Table 1A and 1B, below.

As defined above and described herein, e is 0 or 1.

In some embodiments, e is 0. In some embodiments, e is 1.

In some embodiments, e is selected from those depicted in Table 1A and 1B, below.

As defined above and described herein, X is —O—, —N(R)—, or —S—.

In some embodiments, X is —O—. In some embodiments, X is —N(R)—. In some embodiments, X is —S—In some embodiments, X is selected from those depicted in Table 1A and 1B, below.

As defined above and described herein, Y is O, N (R), or S.

In some embodiments, Y is O. In some embodiments, Y is N (R). In some embodiments, Y is S.

In some embodiments, Y is selected from those depicted in Table 1A and 1B, below.

In some embodiments, KBM is

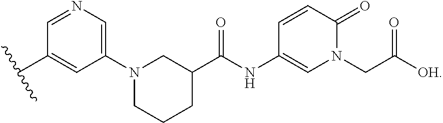 [SMILES: *c1cncc(N2CCCC(C(=O)Nc3ccc(=O)n(CC(=O)O)c3)C2)c1]

In some embodiments, KBM is

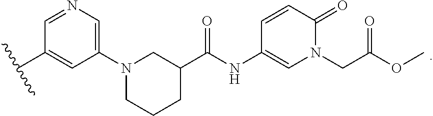 [SMILES: *c1cncc(N2CCCC(C(=O)Nc3ccc(=O)n(CC(=O)OC)c3)C2)c1]

In In some embodiments, KBM is

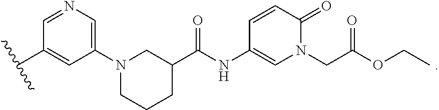 [SMILES: *c1cncc(N2CCCC(C(=O)Nc3ccc(=O)n(CC(=O)OCC)c3)C2)c1]

In some embodiments, KBM is

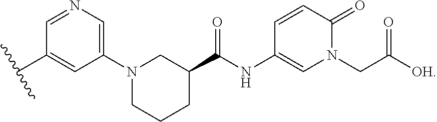 [SMILES: *c1cncc(N2CCC[C@H](C(=O)Nc3ccc(=O)n(CC(=O)O)c3)C2)c1]

In some embodiments, KBM is

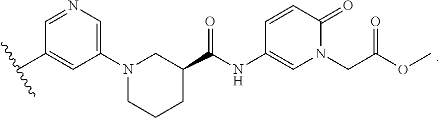 [SMILES: *c1cncc(N2CCC[C@H](C(=O)Nc3ccc(=O)n(CC(=O)OC)c3)C2)c1]

In some embodiments, KBM is

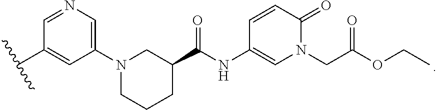 [SMILES: *c1cncc(N2CCC[C@H](C(=O)Nc3ccc(=O)n(CC(=O)OCC)c3)C2)c1]

In some embodiments, KBM is

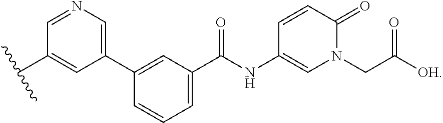 [SMILES: *c1cncc(-c2cccc(C(=O)Nc3ccc(=O)n(CC(=O)O)c3)c2)c1]

In some embodiments, KBM is

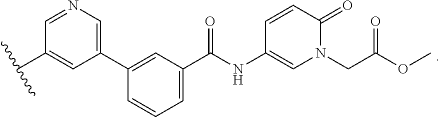 [SMILES: *c1cncc(-c2cccc(C(=O)Nc3ccc(=O)n(CC(=O)OC)c3)c2)c1]

In some embodiments, KBM is

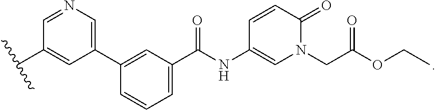 [SMILES: *c1cncc(-c2cccc(C(=O)Nc3ccc(=O)n(CC(=O)OCC)c3)c2)c1]

In some embodiments, KBM is

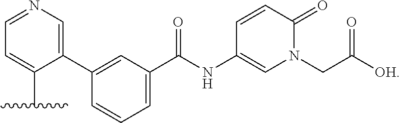 [SMILES: *c1ccncc1-c1cccc(C(=O)Nc2ccc(=O)n(CC(=O)O)c2)c1]

In some embodiments, KBM is

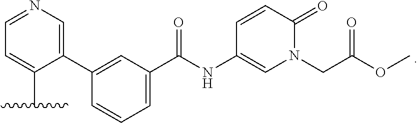 [SMILES: *c1ccncc1-c1cccc(C(=O)Nc2ccc(=O)n(CC(=O)OC)c2)c1]

In some embodiments, KBM is

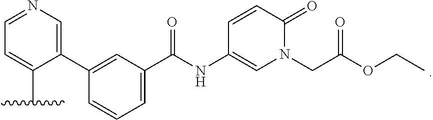 [SMILES: *c1ccncc1-c1cccc(C(=O)Nc2ccc(=O)n(CC(=O)OCC)c2)c1]

In some embodiments, KBM is

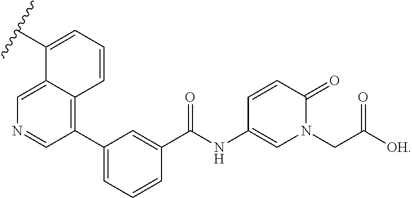 [SMILES: *c1cccc2c(-c3cccc(C(=O)Nc4ccc(=O)n(CC(=O)O)c4)c3)cncc12]

In some embodiments, KBM is

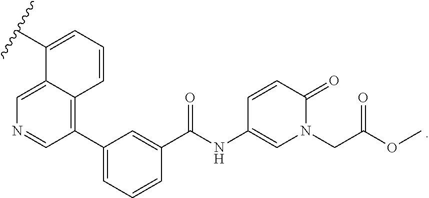 [SMILES: *c1cccc2c(-c3cccc(C(=O)Nc4ccc(=O)n(CC(=O)OC)c4)c3)cncc12]

In some embodiments, KBM is

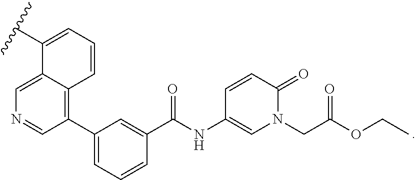 [SMILES: *c1cccc2c(-c3cccc(C(=O)Nc4ccc(=O)n(CC(=O)OCC)c4)c3)cncc12]

In some embodiments, KBM is

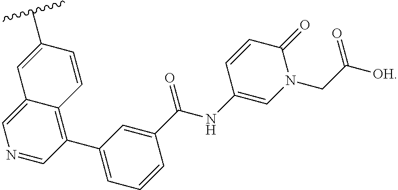 [SMILES: *c1ccc2c(-c3cccc(C(=O)Nc4ccc(=O)n(CC(=O)O)c4)c3)cncc2c1]

In some embodiments, KBM is

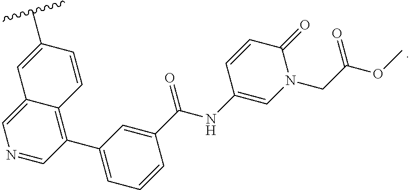 [SMILES: *c1ccc2c(-c3cccc(C(=O)Nc4ccc(=O)n(CC(=O)OC)c4)c3)cncc2c1]

In some embodiments, KBM is

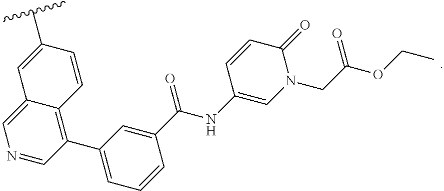 [SMILES: *c1ccc2c(-c3cccc(C(=O)Nc4ccc(=O)n(CC(=O)OCC)c4)c3)cncc2c1]

In some embodiments, KBM is

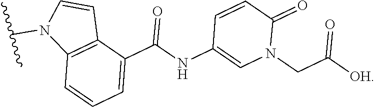 [SMILES: *n1ccc2c(C(=O)Nc3ccc(=O)n(CC(=O)O)c3)cccc21]

In some embodiments, KBM is

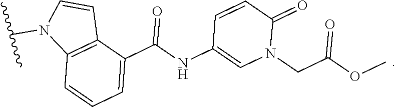 [SMILES: *n1ccc2c(C(=O)Nc3ccc(=O)n(CC(=O)OC)c3)cccc21]

In some embodiments, KBM is

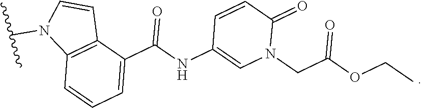 [SMILES: *n1ccc2c(C(=O)Nc3ccc(=O)n(CC(=O)OCC)c3)cccc21]

In some embodiments, KBM is

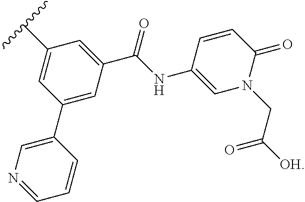 [SMILES: *c1cc(C(=O)Nc2ccc(=O)n(CC(=O)O)c2)cc(-c2cccnc2)c1]

In some embodiments, KBM is

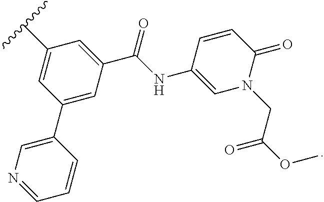 [SMILES: *c1cc(C(=O)Nc2ccc(=O)n(CC(=O)OC)c2)cc(-c2cccnc2)c1]

In some embodiments, KBM is

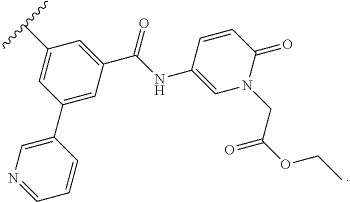 [SMILES: *c1cc(C(=O)Nc2ccc(=O)n(CC(=O)OCC)c2)cc(-c2cccnc2)c1]

In some embodiments, KBM is

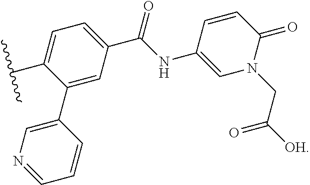 [SMILES: *c1ccc(C(=O)Nc2ccc(=O)n(CC(=O)O)c2)cc1-c1cccnc1]

In some embodiments, KBM is

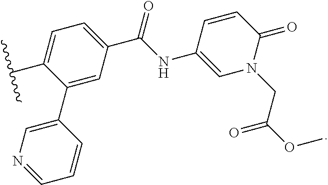 [SMILES: *c1ccc(C(=O)Nc2ccc(=O)n(CC(=O)OC)c2)cc1-c1cccnc1]

In some embodiments, KBM is

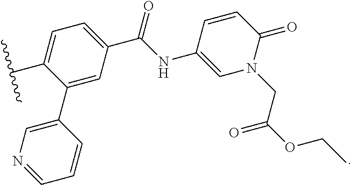 [SMILES: *c1ccc(C(=O)Nc2ccc(=O)n(CC(=O)OCC)c2)cc1-c1cccnc1]

In some embodiments, KBM is

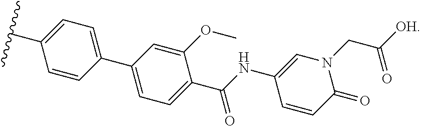 [SMILES: *c1ccc(-c2ccc(C(=O)Nc3ccc(=O)n(CC(=O)O)c3)c(OC)c2)cc1]

In some embodiments, KBM is

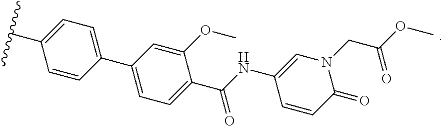 [SMILES: *c1ccc(-c2ccc(C(=O)Nc3ccc(=O)n(CC(=O)OC)c3)c(OC)c2)cc1]

In some embodiments, KBM is

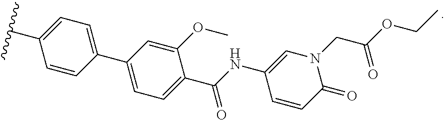 [SMILES: *c1ccc(-c2ccc(C(=O)Nc3ccc(=O)n(CC(=O)OCC)c3)c(OC)c2)cc1]

In some embodiments, KBM is

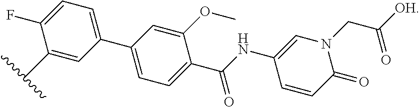 [SMILES: *c1cc(-c2ccc(C(=O)Nc3ccc(=O)n(CC(=O)O)c3)c(OC)c2)ccc1F]

In some embodiments, KBM is

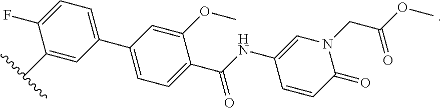 [SMILES: *c1cc(-c2ccc(C(=O)Nc3ccc(=O)n(CC(=O)OC)c3)c(OC)c2)ccc1F]

In some embodiments, KBM is

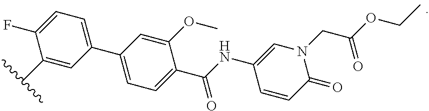 [SMILES: *c1cc(-c2ccc(C(=O)Nc3ccc(=O)n(CC(=O)OCC)c3)c(OC)c2)ccc1F]

In some embodiments, KBM is

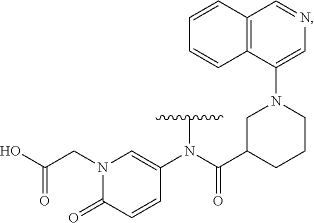 [SMILES: *N(C(=O)C1CCCN(c2cncc3ccccc23)C1)c1ccc(=O)n(CC(=O)O)c1]

In some embodiments, KBM is selected from those depicted in Table 1B, below.

In certain embodiments, the present invention provides a compound of formula I-a, wherein R^{1a} and R^{1b} are hydrogen, d is 1, X is —O—, and Y is O as shown below to provide a compound of formula I-a-1:

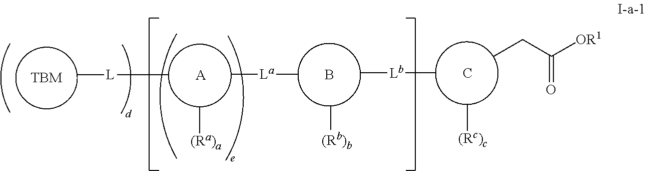

or a pharmaceutically acceptable salt thereof, wherein each of R¹, R^{a}, R^{b}, R^{c}, Ring A, Ring B, Ring C, L^{a}, L^{b}, a, b, c, e, L, and TBM is as defined and described herein, both independently and in combination.

In certain embodiments, the present invention provides a compound of formula I-a, wherein R^{1a} and R^{1b} are hydrogen, d is 1, X is —O—, Y is O, and Ring C is phenylenyl as shown below to provide a compound of formula I-a-2:

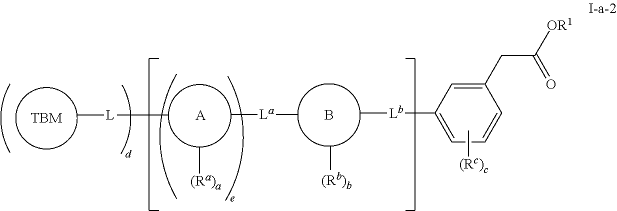

or a pharmaceutically acceptable salt thereof, wherein each of R¹, R^{a}, R^{b}, Re, Ring A, Ring B, L^{a}, L^{b}, a, b, c, e, L, and TBM is as defined and described herein, both independently and in combination.

In certain embodiments, the present invention provides a compound of formula I-a, wherein R^{1a} and R^{1b} are hydrogen, d is 1, X is —O—, Y is O, and Ring C is 2-pyridonyl as shown below to provide a compound of formula I-a-3:

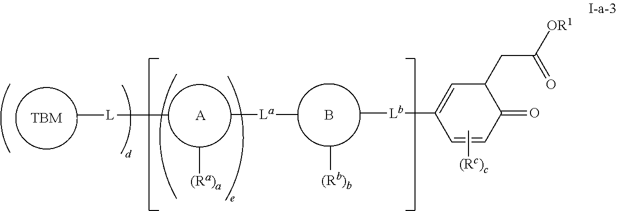

or a pharmaceutically acceptable salt thereof, wherein each of R¹, R^{a}, R^{b}, R^{c}, Ring A, Ring B, L^{a}, L^{b}, a, b, c, e, L, and TBM is as defined and described herein, both independently and in combination.

In certain embodiments, the present invention provides a compound of formula I-a, wherein R^{1a} and R^{1b} are hydrogen, d is 1, X is —O—, Y is O, and L′ is —C(O)NH— as shown below to provide a compound of formula I-a-4:

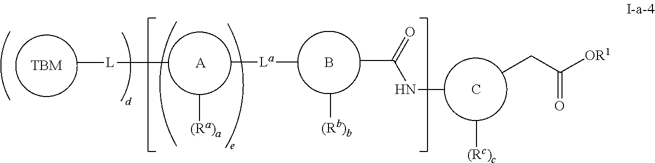

or a pharmaceutically acceptable salt thereof, wherein each of R¹, R^{a}, R^{b}, R^{c}, Ring A, Ring B, Ring C, L^{a}, a, b, c, e, L, and TBM is as defined and described herein, both independently and in combination.

In certain embodiments, the present invention provides a compound of formula I-a, wherein R^{1a} and R^{1b} are hydrogen, d is 1, X is —O—, Y is O, L^{b} is —C(O)NH—, and Ring C is phenylenyl as shown below to provide a compound of formula I-a-5:

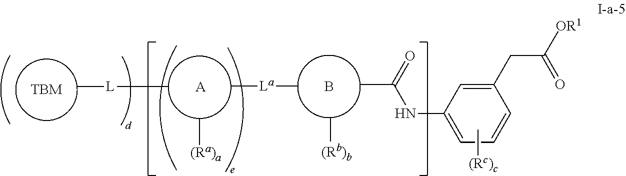

or a pharmaceutically acceptable salt thereof, wherein each of R¹, R^{a}, R^{b}, R^{c}, Ring A, Ring B, L^{a}, L^{b}, a, b, c, e, L, and TBM is as defined and described herein, both independently and in combination.

In certain embodiments, the present invention provides a compound of formula I-a, wherein R^{1a} and R^{1b} are hydrogen, d is 1, X is —O—, Y is O, L′ is —C(O)NH—, and Ring C is 2-pyridonyl as shown below to provide a compound of formula I-a-6:

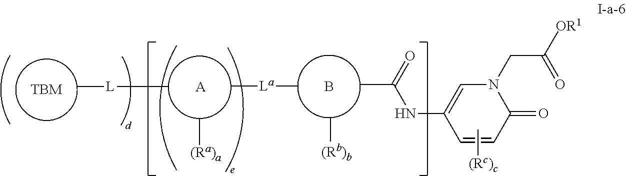

or a pharmaceutically acceptable salt thereof, wherein each of R¹, R^{a}, R^{b}, Re, Ring A, Ring B, L^{a}, L^{b}, a, b, c, e, L, and TBM is as defined and described herein, both independently and in combination.

### Linker (L)

As defined above and described herein, L is a covalent bond or a bivalent, saturated or unsaturated, straight or branched C₁₋₅₀ hydrocarbon chain, wherein 0-6 methylene units of L are independently replaced by —Cy—, —CRF—, —CF₂—, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N (R)—, —N (R)C(O)—, —C(O)N(R)—, —OC(O)N(R)—, —N(R)C(O)O—,

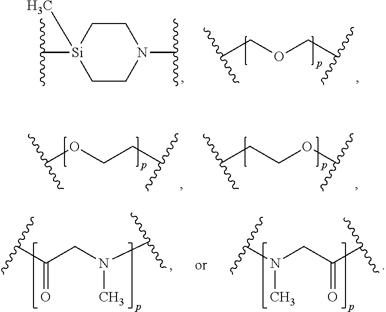

In some embodiments, L is a covalent bond. In some embodiments, L is a bivalent, saturated or unsaturated, straight or branched C₁₋₅₀ hydrocarbon chain, wherein 0-6 methylene units of L are independently replaced by —Cy—, —CRF—, —CF₂—, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N (R)—, —N (R)C(O)—, —C(O)N(R)—, —OC(O)N(R)—, —N(R)C(O)O—,

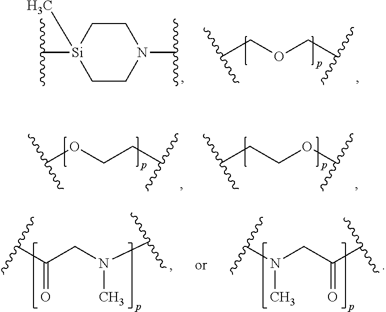

As defined above and described herein, each —Cy— is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur.

In some embodiments, —Cy— is an optionally substituted phenylenyl. In some embodiments, —Cy— is an optionally substituted 8-10 membered bicyclic arylenyl. In some embodiments, —Cy— is an optionally substituted 4-7 membered saturated or partially unsaturated carbocyclylenyl. In some embodiments, —Cy— is an optionally substituted 4-11 membered saturated or partially unsaturated spiro carbocyclylenyl. In some embodiments, —Cy— is an optionally substituted 8-10 membered bicyclic saturated or partially unsaturated carbocyclylenyl. In some embodiments, —Cy— is an optionally substituted 4-7 membered saturated or partially unsaturated heterocyclylenyl having 1-2 heteroatoms independently selected from nitrogen, oxygen, and sulfur. In some embodiments, —Cy— is an optionally substituted 4-11 membered saturated or partially unsaturated spiro heterocyclylenyl having 1-2 heteroatoms independently selected from nitrogen, oxygen, and sulfur. In some embodiments, —Cy— is an optionally substituted 8-10 membered bicyclic saturated or partially unsaturated heterocyclylenyl having 1-2 heteroatoms independently selected from nitrogen, oxygen, and sulfur. In some embodiments, —Cy— is an optionally substituted 5-6 membered heteroarylenyl having 1-4 heteroatoms independently selected from nitrogen, oxygen, and sulfur. In some embodiments, —Cy— is an optionally substituted 8-10 membered bicyclic heteroarylenyl having 1-5 heteroatoms independently selected from nitrogen, oxygen, or sulfur.

In some embodiments, —Cy— is

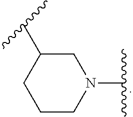 [SMILES: *C1CCCN(*)C1]

In some embodiments, —Cy— is

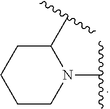 [SMILES: *C1CCCCN1*]

In some embodiments, —Cy— is

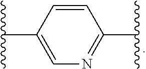 [SMILES: *c1ccc(*)nc1]

In some embodiments, —Cy— is

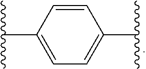 [SMILES: *c1ccc(*)cc1]

In some embodiments, —Cy— is

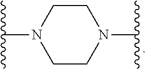 [SMILES: *N1CCN(*)CC1]

In some embodiments, —Cy— is

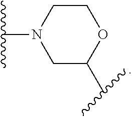 [SMILES: *C1CN(*)CCO1]

In some embodiments, Cy— is

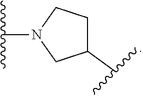 [SMILES: *C1CCN(*)C1]

In some embodiments, —Cy— is

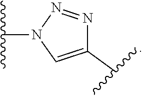 [SMILES: *c1cn(*)nn1]

In some embodiments, —Cy— is

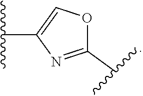 [SMILES: *c1coc(*)n1]

In some embodiments, —Cy— is

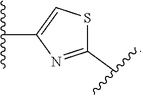 [SMILES: *c1csc(*)n1]

In some embodiments, —Cy— is

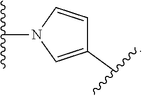 [SMILES: *c1ccn(*)c1]

In some embodiments, —Cy— is

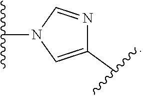 [SMILES: *c1cn(*)cn1]

In some embodiments, —Cy— is

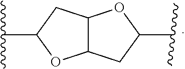 [SMILES: *C1CC2OC(*)CC2O1]

In some embodiments, —Cy— is

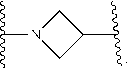 [SMILES: *C1CN(*)C1]

In some embodiments, —Cy— is

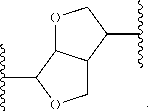 [SMILES: *C1COC2C(*)OCC12]

In some embodiments, —Cy— is

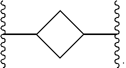 [SMILES: *C1CC(*)C1]

In some embodiments, —Cy— is

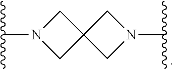 [SMILES: *N1CC2(C1)CN(*)C2]

In some embodiments, —Cy— is

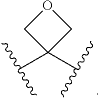 [SMILES: *C1(*)COC1]

In some embodiments, —Cy— is

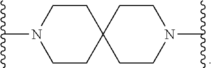 [SMILES: *N1CCC2(CC1)CCN(*)CC2]

In some embodiments, —Cy— is

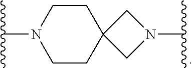 [SMILES: *N1CCC2(CC1)CN(*)C2]

In some embodiments, —Cy— is

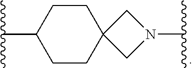 [SMILES: *C1CCC2(CC1)CN(*)C2]

In some embodiments, —Cy— is

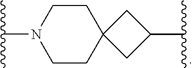 [SMILES: *C1CC2(CCN(*)CC2)C1]

In some embodiments, —Cy— is

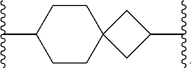 [SMILES: *C1CCC2(CC1)CC(*)C2]

In some embodiments, —Cy— is

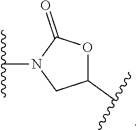 [SMILES: *C1CN(*)C(=O)O1]

In some embodiments, —Cy— is

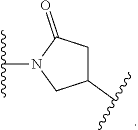 [SMILES: *C1CC(=O)N(*)C1]

In some embodiments, —Cy— is

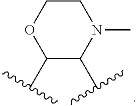 [SMILES: *C1OCCN(C)C1*]

In some embodiments, —Cy— is

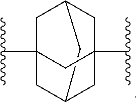 [SMILES: *C12CC3CC(C1)CC(*)(C3)C2]

In some embodiments, —Cy— is

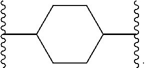 [SMILES: *C1CCC(*)CC1]

In some embodiments, —Cy— is

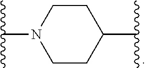 [SMILES: *C1CCN(*)CC1]

In some embodiments, —Cy— is

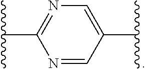 [SMILES: *c1cnc(*)nc1]

In some embodiments, —Cy— is

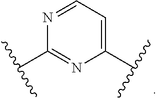 [SMILES: *c1ccnc(*)n1]

In some embodiments, —Cy— is

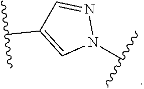 [SMILES: *c1cnn(*)c1]

In some embodiments, —Cy— is

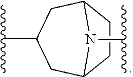 [SMILES: *C1CC2CCC(C1)N2*]

In some embodiments, —Cy— is

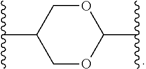 [SMILES: *C1COC(*)OC1]

In some embodiments, —Cy— is

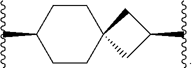

In some embodiments, —Cy— is

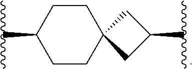

In some embodiments, —Cy— is

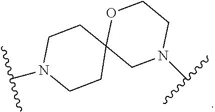 [SMILES: *N1CCC2(CC1)CN(*)CCO2]

In some embodiments, —Cy— is

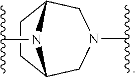 [SMILES: *N1C[C@H]2CC[C@@H](C1)N2*]

In some embodiments, —Cy— is

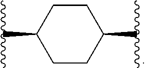 [SMILES: *[C@H]1CC[C@@H](*)CC1]

In some embodiments, —Cy— is

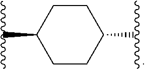 [SMILES: *[C@H]1CC[C@H](*)CC1]

In some embodiments, —Cy— is

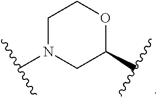 [SMILES: *[C@H]1CN(*)CCO1]

In some embodiments, —Cy— is

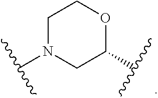 [SMILES: *[C@@H]1CN(*)CCO1]

In some embodiments, —Cy— is

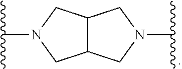 [SMILES: *N1CC2CN(*)CC2C1]

In some embodiments, —Cy— is

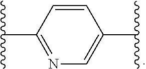 [SMILES: *c1ccc(*)nc1]

In some embodiments, —Cy— is

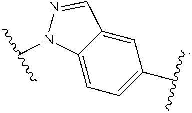 [SMILES: *c1ccc2c(cnn2*)c1]

In some embodiments, —Cy— is selected from those depicted in Table 1, below.

As defined above and described herein, each p is independently 1, 2, 3, 4, 5, 6, 7, 8, 9, or 10.

In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10.

In some embodiments, p is selected from those depicted in Table 1, below.

In some embodiments, L is —NR—(C₁₋₁₀ aliphatic)-. In some embodiments, L is —(C₁₋₁₀ aliphatic)-NR—(C₁₋₁₀aliphatic)-. In some embodiments, L is —(C₁₋₁₀ aliphatic)-NR—(CH₂CH₂O)₁₋₁₀CH₂CH₂—. In some embodiments, L is —Cy—NR—(C₁₋₁₀ aliphatic)-. In some embodiments, L is —Cy—(C₁₋₁₀ aliphatic)-NR—. In some embodiments, L is —Cy—(C₁₋₁₀ aliphatic)-NR—(C₁₋₁₀ aliphatic)-. In some embodiments, L is —(C₁₋₁₀ aliphatic) —Cy—NR—(C₁₋₁₀ aliphatic)-. In some embodiments, L is —(C₁₋₁₀ aliphatic) —Cy—(C₁₋₁₀ aliphatic)-NR—. In some embodiments, L is —(C₁₋₁₀ aliphatic) —Cy—(C₁₋₁₀ aliphatic)-NR—(C₁₋₁₀ aliphatic)-. In some embodiments, L is —Cy—(C₁₋₁₀ aliphatic) —Cy—NR—. In some embodiments, L is —Cy—(C₁₋₁₀ aliphatic)-NR—Cy—. In some embodiments, L is —Cy—(C₁₋₁₀ aliphatic) —Cy—NR—(C₁₋₁₀ aliphatic)-. In some embodiments, L is —Cy—(C₁₋₁₀ aliphatic)-NR—Cy—(C₁₋₁₀ aliphatic)-.

In some embodiments, L is —CONR—(C₁₋₁₀ aliphatic)-. In some embodiments, L is —(C₁₋₁₀ aliphatic)-CONR—(C₁₋₁₀aliphatic)-. In some embodiments, L is —(C₁₋₁₀ aliphatic)-CONR—(CH₂CH₂O)₁₋₁₀CH₂CH₂—. In some embodiments, L is —Cy-CONR—(C₁₋₁₀ aliphatic)-. In some embodiments, L is —Cy—(C₁₋₁₀ aliphatic)-CONR—. In some embodiments, L is —Cy—(C₁₋₁₀ aliphatic)-CONR—(C₁₋₁₀ aliphatic)-. In some embodiments, L is —(C₁₋₁₀ aliphatic) —Cy—CONR—(C₁₋₁₀ aliphatic)-. In some embodiments, L is —(C₁₋₁₀ aliphatic) —Cy—(C₁₋₁₀ aliphatic)-CONR—. In some embodiments, L is —(C₁₋₁₀ aliphatic) —Cy—(C₁₋₁₀ aliphatic)-CONR—(C₁₋₁₀ aliphatic)-. In some embodiments, L is —Cy—(C₁₋₁₀ aliphatic) —Cy—CONR—. In some embodiments, L is —Cy—(C₁₋₁₀ aliphatic)-CONR—Cy—. In some embodiments, L is —Cy—(C₁₋₁₀ aliphatic) —Cy—CONR—(C₁₋₁₀ aliphatic)-. In some embodiments, L is —Cy—(C₁₋₁₀ aliphatic)-CONR—Cy—(C₁₋₁₀ aliphatic)-.

In some embodiments, L is —NRCO—(C₁₋₁₀ aliphatic)-. In some embodiments, L is —(C₁₋₁₀ aliphatic)-NRCO—(C₁₋₁₀aliphatic)-. In some embodiments, L is —(C₁₋₁₀ aliphatic)-NRCO—(CH₂CH₂O)₁₋₁₀CH₂CH₂—. In some embodiments, L is —Cy—NRCO—(C₁₋₁₀ aliphatic)-. In some embodiments, L is —Cy—(C₁-10 aliphatic)-NRCO—. In some embodiments, L is —Cy—(C₁₋₁₀ aliphatic)-NRCO—(C₁₋₁₀ aliphatic)-. In some embodiments, L is —(C₁₋₁₀ aliphatic) —Cy—NRCO—(C₁₋₁₀ aliphatic)-. In some embodiments, L is —(C₁₋₁₀ aliphatic) —Cy—(C₁₋₁₀ aliphatic)-NRCO—. In some embodiments, L is —(C₁₋₁₀ aliphatic) —Cy—(C₁₋₁₀ aliphatic)-NRCO—(C₁₋₁₀ aliphatic)-. In some embodiments, L is —Cy—(C₁₋₁₀ aliphatic) —Cy—NRCO—. In some embodiments, L is —Cy—(C₁₋₁₀ aliphatic)-NRCO—Cy—. In some embodiments, L is —Cy—(C₁₋₁₀ aliphatic) —Cy—NRCO—(C₁₋₁₀ aliphatic)-. In some embodiments, L is —Cy—(C₁₋₁₀ aliphatic)-NRCO—Cy—(C₁₋₁₀ aliphatic)-.

In some embodiments, L is —O—(C₁₋₁₀ aliphatic)-. In some embodiments, L is —(C₁₋₁₀ aliphatic) —O—(C₁₋₁₀aliphatic)-. In some embodiments, L is —(C₁₋₁₀ aliphatic) —O—(CH₂CH₂O)₁₋₁₀CH₂CH₂—. In some embodiments, L is —Cy—O—(C₁₋₁₀ aliphatic)-. In some embodiments, L is —Cy—(C₁₋₁₀ aliphatic) —O—. In some embodiments, L is —Cy—(C₁₋₁₀ aliphatic) —O—(C₁-10 aliphatic)-. In some embodiments, L is —(C₁₋₁₀ aliphatic) —Cy—O—(C₁₋₁₀ aliphatic)-. In some embodiments, L is —(C₁₋₁₀ aliphatic) —Cy—(C₁₋₁₀ aliphatic) —O—. In some embodiments, L is —(C₁₋₁₀ aliphatic) —Cy—(C₁₋₁₀ aliphatic) —O—(C₁-10 aliphatic)-. In some embodiments, L is —Cy—(C₁₋₁₀ aliphatic) —Cy—O—. In some embodiments, L is —Cy—(C₁₋₁₀ aliphatic)-O—Cy—. In some embodiments, L is —Cy—(C₁₋₁₀ aliphatic) —Cy—O—(C₁₋₁₀ aliphatic)-. In some embodiments, L is —Cy—(C₁₋₁₀ aliphatic) —O—Cy—(C₁₋₁₀ aliphatic)-.

In some embodiments, L is —Cy—(C₁₋₁₀ aliphatic)-. In some embodiments, L is —(C₁₋₁₀ aliphatic) —Cy—(C₁₋₁₀ aliphatic)-. In some embodiments, L is —(C₁₋₁₀ aliphatic) —Cy—(CH₂CH₂O)₁₋₁₀CH₂CH₂—. In some embodiments, L is —Cy—(C₁₋₁₀ aliphatic) —Cy—. In some embodiments, L is —Cy—(C₁₋₁₀ aliphatic) —Cy—(C₁₋₁₀ aliphatic)-. In some embodiments, L is —Cy—(C₁₋₁₀ aliphatic) —Cy—(C₁₋₁₀ aliphatic) —Cy—. In some embodiments, L is —(C₁₋₁₀ aliphatic) —Cy—(C₁₋₁₀ aliphatic) —Cy—(C₁₋₁₀ aliphatic)-.

In some embodiments, L is —NR—(CH₂)₁₋₁₀—. In some embodiments, L is —(CH₂)₁₋₁₀—NR—(CH₂)₁₋₁₀—. In some embodiments, L is —(CH₂)₁₋₁₀—NR—(CH₂CH₂O)₁₋₁₀CH₂CH₂—. In some embodiments, L is —Cy—NR—(CH₂)₁₋₁₀—. In some embodiments, L is —Cy—(CH₂)₁₋₁₀—NR—. In some embodiments, L is —Cy—(CH₂)₁₋₁₀—NR—(CH₂)₁₋₁₀—. In some embodiments, L is —(CH₂)₁₋₁₀—Cy—NR—(CH₂)₁₋₁₀—. In some embodiments, L is —(CH₂)₁₋₁₀—Cy—(CH₂)₁₋₁₀—NR—. In some embodiments, L is —(CH₂)₁₋₁₀—Cy—(CH₂)₁₋₁₀—NR—(CH₂)₁₋₁₀—. In some embodiments, L is —Cy—(CH₂)₁₋₁₀—Cy—NR—. In some embodiments, L is —Cy—(CH₂)₁₋₁₀—NR—Cy—. In some embodiments, L is —Cy—(CH₂)₁₋₁₀—Cy—NR—(CH₂)₁₋₁₀—. In some embodiments, L is —Cy—(CH₂)₁₋₁₀—NR—Cy—(CH₂)₁₋₁₀—.

In some embodiments, L is —CONR—(CH₂)₁₋₁₀—. In some embodiments, L is —(CH₂)₁₋₁₀—CONR—(CH₂)₁₋₁₀—. In some embodiments, L is —(CH₂)₁₋₁₀—CONR—(CH₂CH₂O)₁₋₁₀CH₂CH₂—. In some embodiments, L is —Cy—CONR—(CH₂)₁₋₁₀—. In some embodiments, L is —Cy—(CH₂)₁₋₁₀—CONR—. In some embodiments, L is —Cy—(CH₂)₁₋₁₀—CONR—(CH₂)₁₋₁₀—. In some embodiments, L is —(CH₂)₁₋₁₀—Cy—CONR—(CH₂)₁₋₁₀—. In some embodiments, L is —(CH₂)₁₋₁₀—Cy—(CH₂)₁₋₁₀—CONR—. In some embodiments, L is —(CH₂)₁₋₁₀—Cy—(CH₂)₁₋₁₀—CONR—(CH₂)₁₋₁₀—. In some embodiments, L is —Cy—(CH₂)₁₋₁₀—Cy—CONR—. In some embodiments, L is —Cy—(CH₂)₁₋₁₀—CONR—Cy—. In some embodiments, L is —Cy—(CH₂)₁₋₁₀—Cy—CONR—(CH₂)₁₋₁₀—. In some embodiments, L is —Cy—(CH₂)₁₋₁₀—CONR—Cy—(CH₂)₁₋₁₀—.

In some embodiments, L is —NRCO—(CH₂)₁₋₁₀—. In some embodiments, L is —(CH₂)₁₋₁₀—NRCO—(CH₂)₁₋₁₀—. In some embodiments, L is —(CH₂)₁₋₁₀—NRCO—(CH₂CH₂O)₁₋₁₀CH₂CH₂—. In some embodiments, L is —Cy—NRCO—(CH₂)₁₋₁₀—. In some embodiments, L is —Cy—(CH₂)₁₋₁₀—NRCO—. In some embodiments, L is —Cy—(CH₂)₁₋₁₀—NRCO—(CH₂)₁₋₁₀—. In some embodiments, L is —(CH₂)₁₋₁₀—Cy—NRCO—(CH₂)₁₋₁₀—. In some embodiments, L is —(CH₂)₁₋₁₀—Cy—(CH₂)₁₋₁₀—NRCO—. In some embodiments, L is —(CH₂)₁₋₁₀—Cy—(CH₂)₁₋₁₀—NRCO—(CH₂)₁₋₁₀—. In some embodiments, L is —Cy—(CH₂)₁₋₁₀—Cy—NRCO—. In some embodiments, L is —Cy—(CH₂)₁₋₁₀—NRCO—Cy—. In some embodiments, L is —Cy—(CH₂)₁₋₁₀—Cy—NRCO—(CH₂)₁₋₁₀—. In some embodiments, L is —Cy—(CH₂)₁₋₁₀—NRCO—Cy—(CH₂)₁₋₁₀—.

In some embodiments, L is —O—(CH₂)₁₋₁₀—. In some embodiments, L is —(CH₂)₁₋₁₀—O—(CH₂)₁₋₁₀—. In some embodiments, L is —(CH₂)₁₋₁₀—O—(CH₂CH₂O)₁₋₁₀CH₂CH₂—. In some embodiments, L is —Cy—O—(CH₂)₁₋₁₀—. In some embodiments, L is —Cy—(CH₂)₁₋₁₀—O—. In some embodiments, L is —Cy—(CH₂)₁₋₁₀—O—(CH₂)₁₋₁₀—. In some embodiments, L is —(CH₂)₁₋₁₀—Cy—O—(CH₂)₁₋₁₀—. In some embodiments, L is —(CH₂)₁₋₁₀—Cy—(CH₂)₁₋₁₀—O—. In some embodiments, L is —(CH₂)₁₋₁₀—Cy—(CH₂)₁₋₁₀—O—(CH₂)₁₋₁₀—. In some embodiments, L is —Cy—(CH₂)₁₋₁₀—Cy—O—. In some embodiments, L is —Cy—(CH₂)₁₋₁₀—O—Cy—. In some embodiments, L is —Cy—(CH₂)₁₋₁₀—Cy—O—(CH₂)₁₋₁₀—. In some embodiments, L is —Cy—(CH₂)₁₋₁₀—O—Cy—(CH₂)₁₋₁₀—.

In some embodiments, L is —Cy—(CH₂)₁₋₁₀—. In some embodiments, L is —(CH₂)₁₋₁₀—Cy—(CH₂)₁₋₁₀—. In some embodiments, L is —(CH₂)₁₋₁₀—Cy—(CH₂CH₂O)₁₋₁₀CH₂CH₂—. In some embodiments, L is —Cy—(CH₂)₁₋₁₀—Cy—. In some embodiments, L is —Cy—(CH₂)₁₋₁₀—Cy—(CH₂)₁₋₁₀—. In some embodiments, L is —Cy—(CH₂)₁₋₁₀—Cy—(CH₂)₁₋₁₀—Cy—. In some embodiments, L is —(CH₂)₁₋₁₀—Cy—(CH₂)₁₋₁₀—Cy—(CH₂)₁₋₁₀—.

In some embodiments, L is

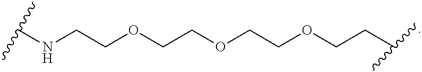 [SMILES: *CCOCCOCCOCCN*]

In some embodiments, L is

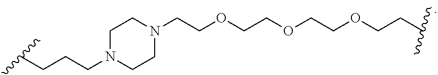 [SMILES: *CCCN1CCN(CCOCCOCCOCC*)CC1]

In some embodiments, L is

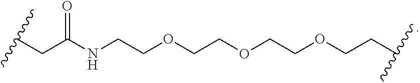 [SMILES: *CCOCCOCCOCCNC(=O)C*]

In some embodiments, L is

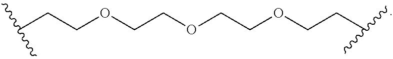 [SMILES: *CCOCCOCCOCC*]

In some embodiments, L is

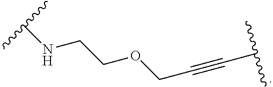 [SMILES: *C#CCOCCN*]

In some embodiments, L is

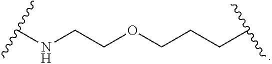 [SMILES: *CCCOCCN*]

In some embodiments, L is

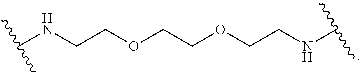 [SMILES: *NCCOCCOCCN*]

In some embodiments, L is

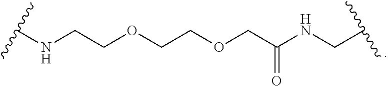 [SMILES: *CNC(=O)COCCOCCN*]

In some embodiments, L is

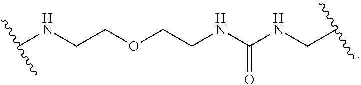 [SMILES: *CNC(=O)NCCOCCN*]

In some embodiments, L is

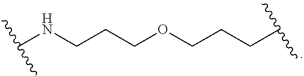 [SMILES: *CCCOCCCN*]

In some embodiments, L is

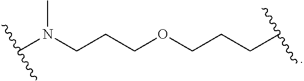 [SMILES: *CCCOCCCN(*)C]

In some embodiments, L is

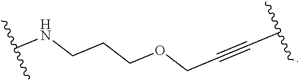 [SMILES: *C#CCOCCCN*]

In some embodiments, L is

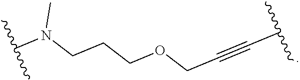 [SMILES: *C#CCOCCCN(*)C]

In some embodiments, L is

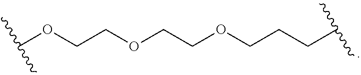 [SMILES: *CCCOCCOCCO*]

In some embodiments, L is

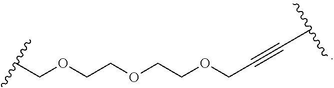 [SMILES: *C#CCOCCOCCOC*]

In some embodiments, L is

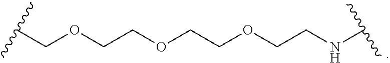 [SMILES: *COCCOCCOCCN*]

In some embodiments, L is

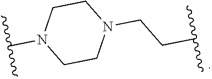 [SMILES: *CCN1CCN(*)CC1]

In some embodiments, L is

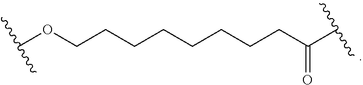 [SMILES: *OCCCCCCCCC(*)=O]

In some embodiments, L is

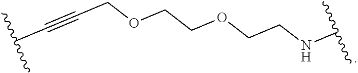 [SMILES: *C#CCOCCOCCN*]

In some embodiments, L is

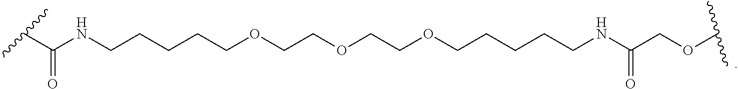 [SMILES: *OCC(=O)NCCCCCOCCOCCOCCCCCNC(*)=O]

In some embodiments, L is

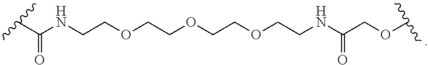 [SMILES: *OCC(=O)NCCOCCOCCOCCNC(*)=O]

In some embodiments, L is

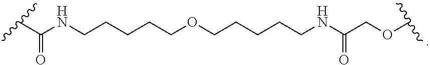 [SMILES: *OCC(=O)NCCCCCOCCCCCNC(*)=O]

In some embodiments, L is

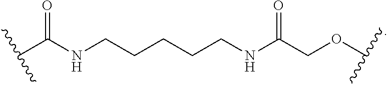 [SMILES: *OCC(=O)NCCCCCNC(*)=O]

In some embodiments, L is

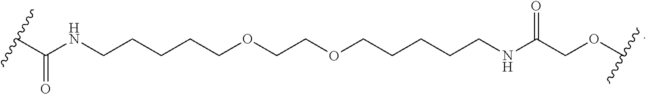 [SMILES: *OCC(=O)NCCCCCOCCOCCCCCNC(*)=O]

In some embodiments, L is

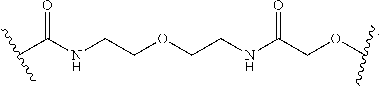 [SMILES: *OCC(=O)NCCOCCNC(*)=O]

In some embodiments, L is

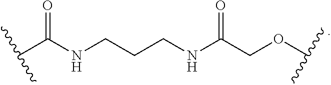 [SMILES: *OCC(=O)NCCCNC(*)=O]

In some embodiments, L is

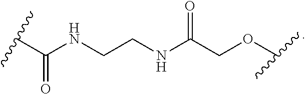 [SMILES: *OCC(=O)NCCNC(*)=O]

In some embodiments, L is

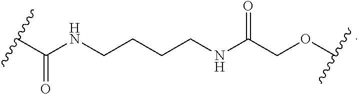 [SMILES: *OCC(=O)NCCCCNC(*)=O]

In some embodiments, L is

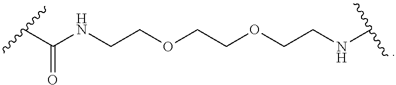 [SMILES: *NCCOCCOCCNC(*)=O]

In some embodiments, L is

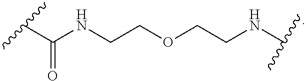 [SMILES: *NCCOCCNC(*)=O]

In some embodiments, L is

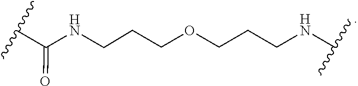 [SMILES: *NCCCOCCCNC(*)=O]

In some embodiments, L is

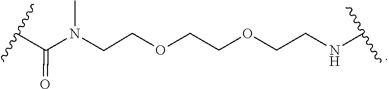 [SMILES: *NCCOCCOCCN(C)C(*)=O]

In some embodiments, L is

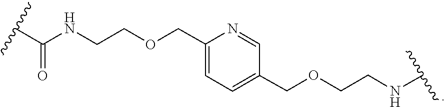 [SMILES: *NCCOCc1ccc(COCCNC(*)=O)nc1]

In some embodiments, L is

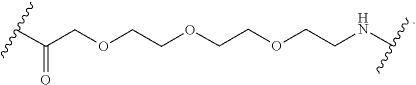 [SMILES: *NCCOCCOCCOCC(*)=O]

In some embodiments, L is

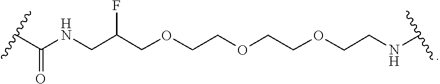 [SMILES: *NCCOCCOCCOCC(F)CNC(*)=O]

In some embodiments, L is

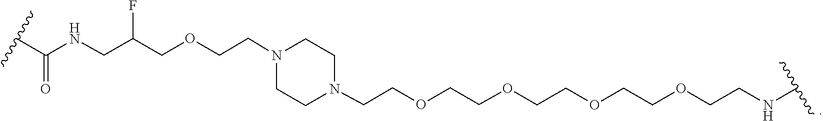 [SMILES: *NCCOCCOCCOCCOCCN1CCN(CCOCC(F)CNC(*)=O)CC1]

In some embodiments, L is

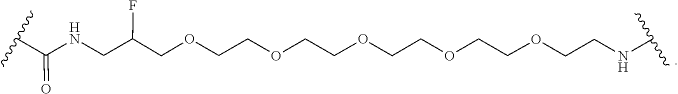 [SMILES: *NCCOCCOCCOCCOCCOCC(F)CNC(*)=O]

In some embodiments, L is

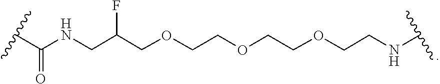 [SMILES: *NCCOCCOCCOCC(F)CNC(*)=O]

In some embodiments, L is

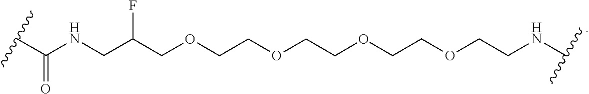 [SMILES: *NCCOCCOCCOCCOCC(F)CNC(*)=O]

In some embodiments, L is

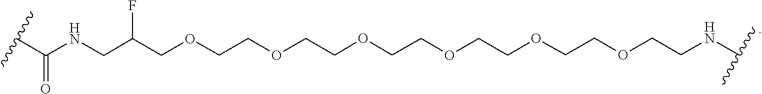 [SMILES: *NCCOCCOCCOCCOCCOCCOCC(F)CNC(*)=O]

In some embodiments, L is

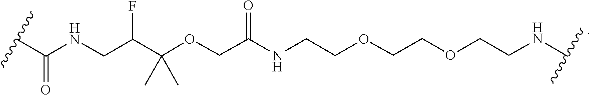 [SMILES: *NCCOCCOCCNC(=O)COC(C)(C)C(F)CNC(*)=O]

In some embodiments, L is

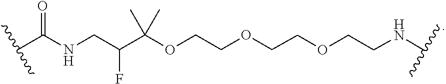 [SMILES: *NCCOCCOCCOC(C)(C)C(F)CNC(*)=O]

In some embodiments, L is

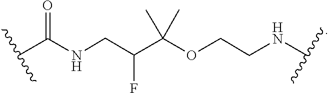 [SMILES: *NCCOC(C)(C)C(F)CNC(*)=O]

In some embodiments, L is

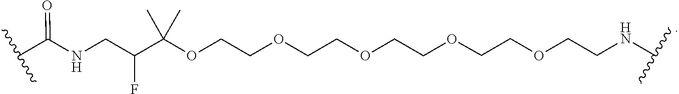 [SMILES: *NCCOCCOCCOCCOCCOC(C)(C)C(F)CNC(*)=O]

In some embodiments, L is

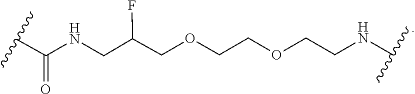 [SMILES: *NCCOCCOCC(F)CNC(*)=O]

In some embodiments, L is

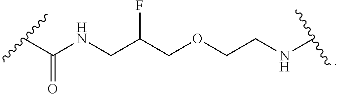 [SMILES: *NCCOCC(F)CNC(*)=O]

In some embodiments, L is

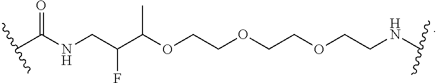 [SMILES: *NCCOCCOCCOC(C)C(F)CNC(*)=O]

In some embodiments, L is

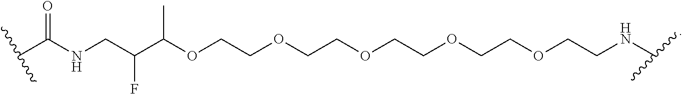 [SMILES: *NCCOCCOCCOCCOCCOC(C)C(F)CNC(*)=O]

In some embodiments, L is

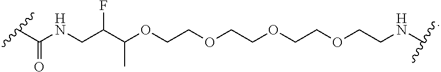 [SMILES: *NCCOCCOCCOCCOC(C)C(F)CNC(*)=O]

In some embodiments, L is

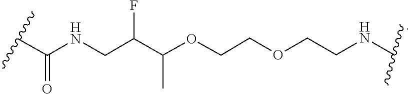 [SMILES: *NCCOCCOC(C)C(F)CNC(*)=O]

In some embodiments, L is

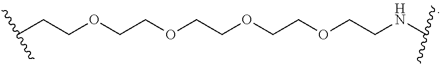 [SMILES: *CCOCCOCCOCCOCCN*]

In some embodiments, L is

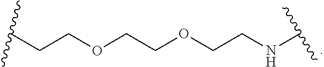 [SMILES: *CCOCCOCCN*]

In some embodiments, L is

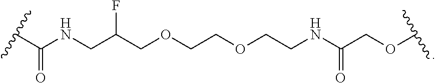 [SMILES: *OCC(=O)NCCOCCOCC(F)CNC(*)=O]

In some embodiments, L is

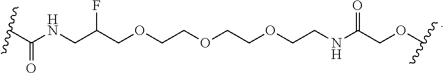 [SMILES: *OCC(=O)NCCOCCOCCOCC(F)CNC(*)=O]

In some embodiments, L is

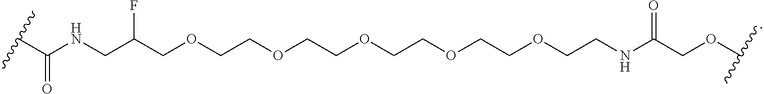 [SMILES: *OCC(=O)NCCOCCOCCOCCOCCOCC(F)CNC(*)=O]

In some embodiments, L is

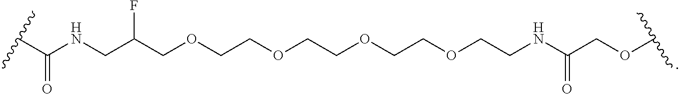 [SMILES: *OCC(=O)NCCOCCOCCOCCOCC(F)CNC(*)=O]

In some embodiments, L is

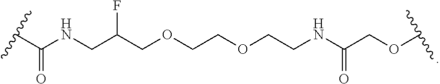 [SMILES: *OCC(=O)NCCOCCOCC(F)CNC(*)=O]

In some embodiments, L is

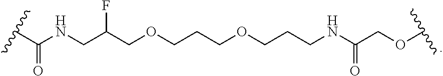 [SMILES: *OCC(=O)NCCCOCCCOCC(F)CNC(*)=O]

In some embodiments, L is

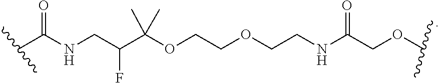 [SMILES: *OCC(=O)NCCOCCOC(C)(C)C(F)CNC(*)=O]

In some embodiments, L is

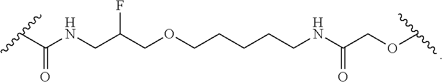 [SMILES: *OCC(=O)NCCCCCOCC(F)CNC(*)=O]

In some embodiments, L is

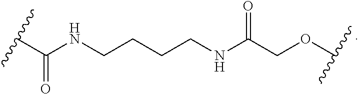 [SMILES: *OCC(=O)NCCCCNC(*)=O]

In some embodiments, L is

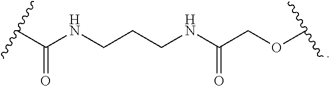 [SMILES: *OCC(=O)NCCCNC(*)=O]

In some embodiments, L is

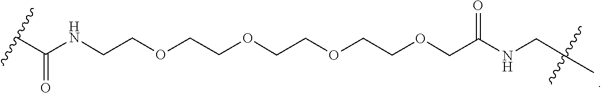 [SMILES: *CNC(=O)COCCOCCOCCOCCNC(*)=O]

In some embodiments, L is

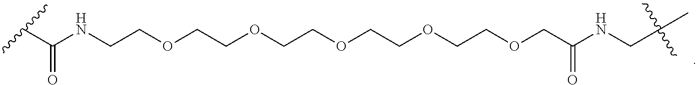 [SMILES: *CNC(=O)COCCOCCOCCOCCOCCNC(*)=O]

In some embodiments, L is

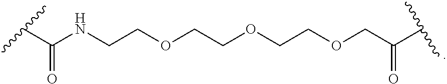 [SMILES: *C(=O)COCCOCCOCCNC(*)=O]

In some embodiments, L is

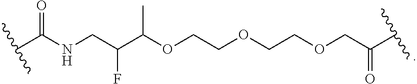 [SMILES: *C(=O)COCCOCCOC(C)C(F)CNC(*)=O]

In some embodiments, L is

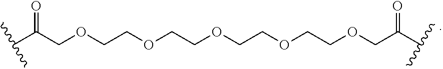 [SMILES: *C(=O)COCCOCCOCCOCCOCC(*)=O]

In some embodiments, L is

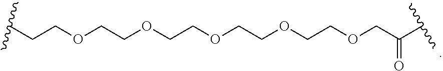 [SMILES: *CCOCCOCCOCCOCCOCC(*)=O]

In some embodiments, L is

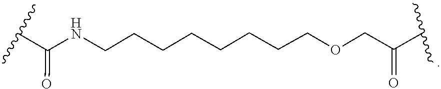 [SMILES: *C(=O)COCCCCCCCCNC(*)=O]

In some embodiments, L is

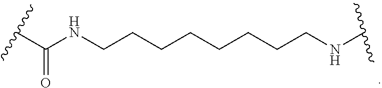 [SMILES: *NCCCCCCCCNC(*)=O]

In some embodiments, L is

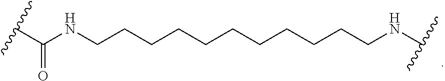 [SMILES: *NCCCCCCCCCCCNC(*)=O]

In some embodiments, L is

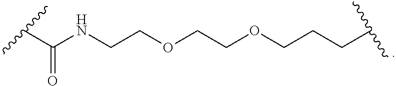 [SMILES: *CCCOCCOCCNC(*)=O]

In some embodiments, L is

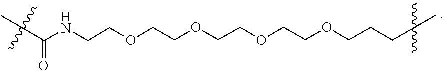 [SMILES: *CCCOCCOCCOCCOCCNC(*)=O]

In some embodiments, L is

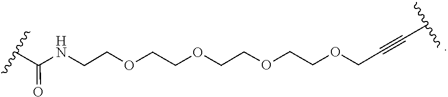 [SMILES: *C#CCOCCOCCOCCOCCNC(*)=O]

In some embodiments, L is

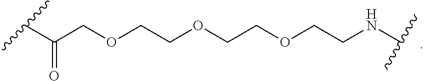 [SMILES: *NCCOCCOCCOCC(*)=O]

In some embodiments, L is

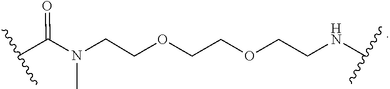 [SMILES: *NCCOCCOCCN(C)C(*)=O]

In some embodiments, L is

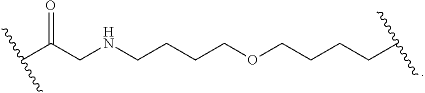 [SMILES: *CCCCOCCCCNCC(*)=O]

In some embodiments, L is

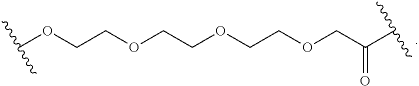 [SMILES: *OCCOCCOCCOCC(*)=O]

In some embodiments, L is

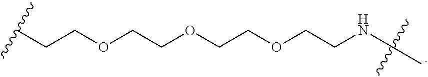 [SMILES: *CCOCCOCCOCCN*]

In some embodiments, L is

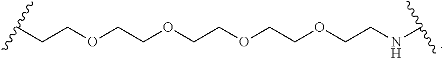 [SMILES: *CCOCCOCCOCCOCCN*]

In some embodiments, L is

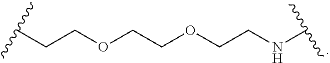 [SMILES: *CCOCCOCCN*]

In some embodiments, L is

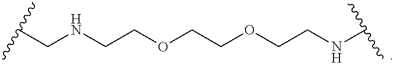 [SMILES: *CNCCOCCOCCN*]

In some embodiments, L is

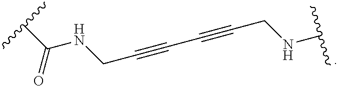 [SMILES: *NCC#CC#CCNC(*)=O]

In some embodiments, L is

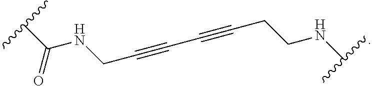 [SMILES: *NCCC#CC#CCNC(*)=O]

In some embodiments, L is

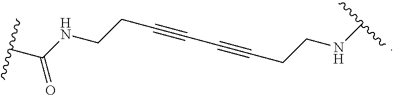 [SMILES: *NCCC#CC#CCCNC(*)=O]

In some embodiments, L is

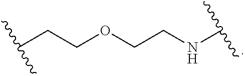 [SMILES: *CCOCCN*]

In some embodiments, L is

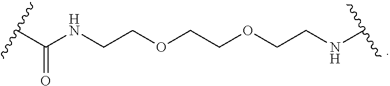 [SMILES: *NCCOCCOCCNC(*)=O]

In some embodiments, L is

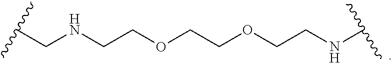 [SMILES: *CNCCOCCOCCN*]

In some embodiments, L is

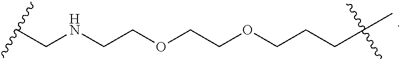 [SMILES: *CCCOCCOCCNC*]

In some embodiments, L is

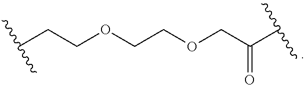 [SMILES: *CCOCCOCC(*)=O]

In some embodiments, L is

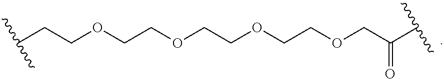 [SMILES: *CCOCCOCCOCCOCC(*)=O]

In some embodiments, L is

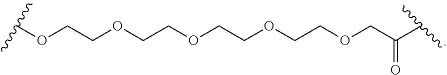 [SMILES: *OCCOCCOCCOCCOCC(*)=O]

In some embodiments, L is

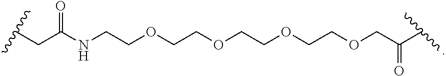 [SMILES: *CC(=O)NCCOCCOCCOCCOCC(*)=O]

In some embodiments, L is

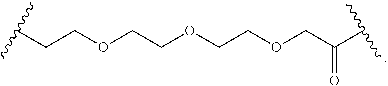 [SMILES: *CCOCCOCCOCC(*)=O]

In some embodiments, L is

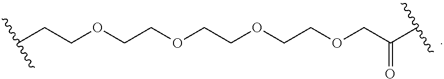 [SMILES: *CCOCCOCCOCCOCC(*)=O]

In some embodiments, L is

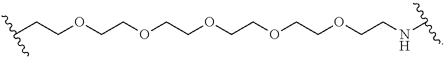 [SMILES: *CCOCCOCCOCCOCCOCCN*]

In some embodiments, L is

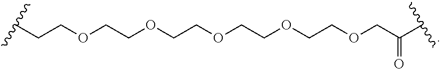 [SMILES: *CCOCCOCCOCCOCCOCC(*)=O]

In some embodiments, L is

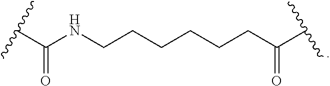 [SMILES: *C(=O)CCCCCCNC(*)=O]

In some embodiments, L is

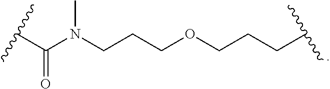 [SMILES: *CCCOCCCN(C)C(*)=O]

In some embodiments, L is

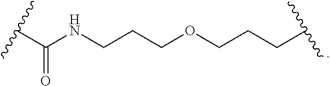 [SMILES: *CCCOCCCNC(*)=O]

In some embodiments, L is

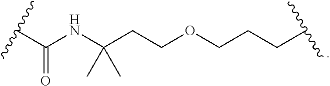 [SMILES: *CCCOCCC(C)(C)NC(*)=O]

In some embodiments, L is

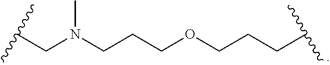 [SMILES: *CCCOCCCN(C)C*]

In some embodiments, L is

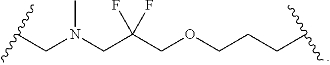 [SMILES: *CCCOCC(F)(F)CN(C)C*]

In some embodiments, L is

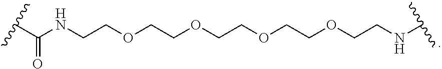 [SMILES: *NCCOCCOCCOCCOCCNC(*)=O]

In some embodiments, L is

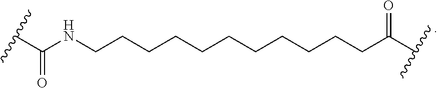 [SMILES: *C(=O)CCCCCCCCCCCNC(*)=O]

In some embodiments, L is

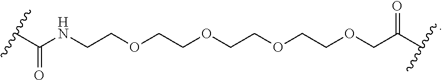 [SMILES: *C(=O)COCCOCCOCCOCCNC(*)=O]

In some embodiments, L is

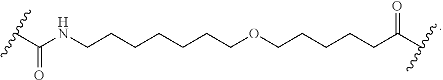 [SMILES: *C(=O)CCCCCOCCCCCCCNC(*)=O]

In some embodiments, L is

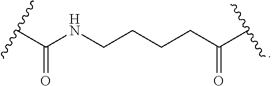 [SMILES: *C(=O)CCCCNC(*)=O]

In some embodiments, L is

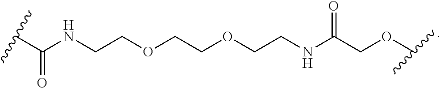 [SMILES: *OCC(=O)NCCOCCOCCNC(*)=O]

In some embodiments, L is

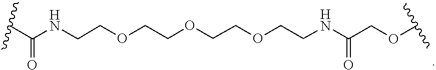 [SMILES: *OCC(=O)NCCOCCOCCOCCNC(*)=O]

In some embodiments, L is

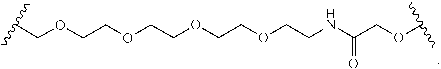 [SMILES: *COCCOCCOCCOCCNC(=O)CO*]

In some embodiments, L is

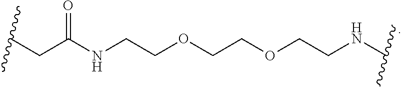 [SMILES: *CC(=O)NCCOCCOCCN*]

In some embodiments, L is

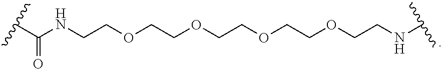 [SMILES: *NCCOCCOCCOCCOCCNC(*)=O]

In some embodiments, L is

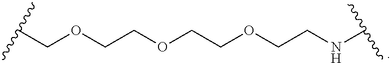 [SMILES: *COCCOCCOCCN*]

In some embodiments, L is

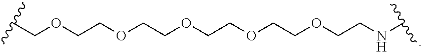 [SMILES: *COCCOCCOCCOCCOCCN*]

In some embodiments, L is

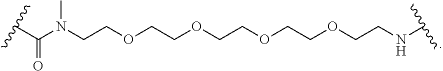 [SMILES: *NCCOCCOCCOCCOCCN(C)C(*)=O]

In some embodiments, L is

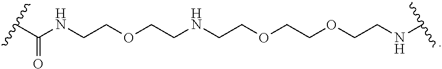 [SMILES: *NCCOCCOCCNCCOCCNC(*)=O]

In some embodiments, L is

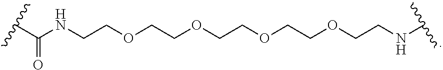 [SMILES: *NCCOCCOCCOCCOCCNC(*)=O]

In some embodiments, L is

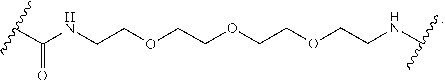 [SMILES: *NCCOCCOCCOCCNC(*)=O]

In some embodiments, L is

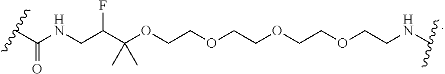 [SMILES: *NCCOCCOCCOCCOC(C)(C)C(F)CNC(*)=O]

In some embodiments, L is

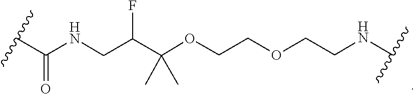 [SMILES: *NCCOCCOC(C)(C)C(F)CNC(*)=O]

In some embodiments, L is

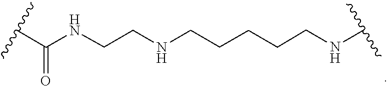 [SMILES: *NCCCCCNCCNC(*)=O]

In some embodiments, L is

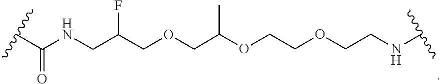 [SMILES: *NCCOCCOC(C)COCC(F)CNC(*)=O]

In some embodiments, L is

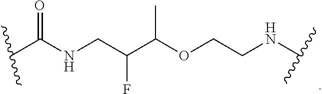 [SMILES: *NCCOC(C)C(F)CNC(*)=O]

In some embodiments, L is

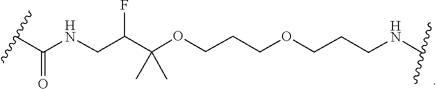 [SMILES: *NCCCOCCCOC(C)(C)C(F)CNC(*)=O]

In some embodiments, L is

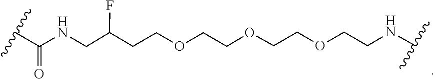 [SMILES: *NCCOCCOCCOCCC(F)CNC(*)=O]

In some embodiments, L is

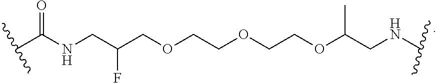 [SMILES: *NCC(C)OCCOCCOCC(F)CNC(*)=O]

In some embodiments, L is

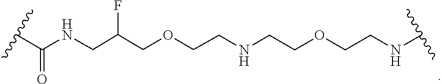 [SMILES: *NCCOCCNCCOCC(F)CNC(*)=O]

In some embodiments, L is

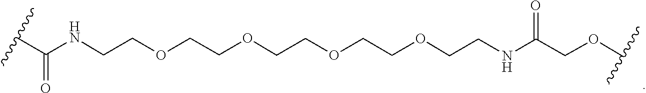 [SMILES: *OCC(=O)NCCOCCOCCOCCOCCNC(*)=O]

In some embodiments, L is

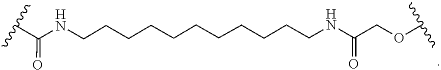 [SMILES: *OCC(=O)NCCCCCCCCCCCNC(*)=O]

In some embodiments, L is

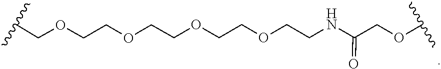 [SMILES: *COCCOCCOCCOCCNC(=O)CO*]

In some embodiments, L is

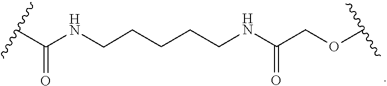 [SMILES: *OCC(=O)NCCCCCNC(*)=O]

In some embodiments, L is

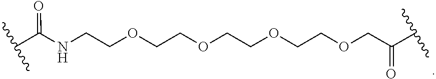 [SMILES: *C(=O)COCCOCCOCCOCCNC(*)=O]

In some embodiments, L is

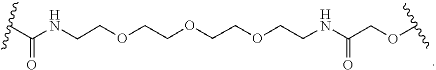 [SMILES: *OCC(=O)NCCOCCOCCOCCNC(*)=O]

In some embodiments, L is

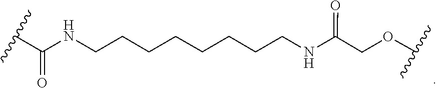 [SMILES: *OCC(=O)NCCCCCCCCNC(*)=O]

In some embodiments, L is

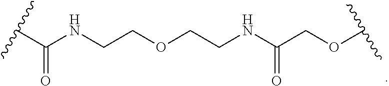 [SMILES: *OCC(=O)NCCOCCNC(*)=O]

In some embodiments, L is

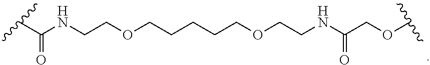 [SMILES: *OCC(=O)NCCOCCCCCOCCNC(*)=O]

In some embodiments, L is

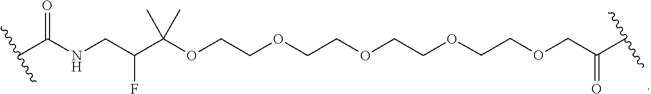 [SMILES: *C(=O)COCCOCCOCCOCCOC(C)(C)C(F)CNC(*)=O]

In some embodiments, L is

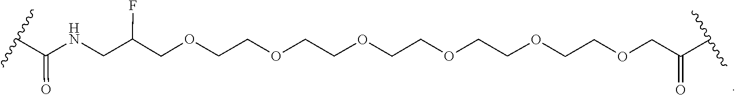 [SMILES: *C(=O)COCCOCCOCCOCCOCCOCC(F)CNC(*)=O]

In some embodiments, L is

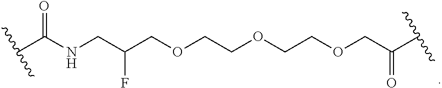 [SMILES: *C(=O)COCCOCCOCC(F)CNC(*)=O]

In some embodiments, L is

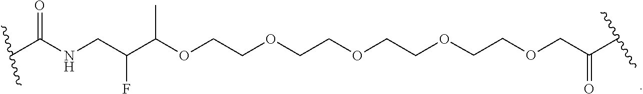 [SMILES: *C(=O)COCCOCCOCCOCCOC(C)C(F)CNC(*)=O]

In some embodiments, L is

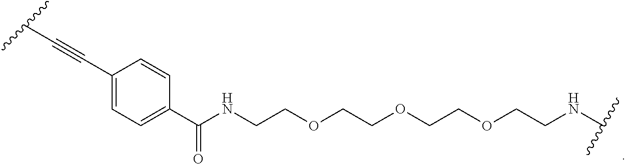 [SMILES: *C#Cc1ccc(C(=O)NCCOCCOCCOCCN*)cc1]

In some embodiments, L is

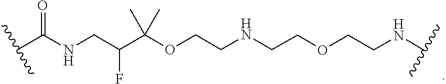 [SMILES: *NCCOCCNCCOC(C)(C)C(F)CNC(*)=O]

In some embodiments, L is

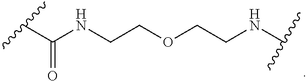 [SMILES: *NCCOCCNC(*)=O]

In some embodiments, L is

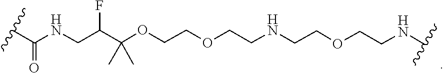 [SMILES: *NCCOCCNCCOCCOC(C)(C)C(F)CNC(*)=O]

In some embodiments, L is

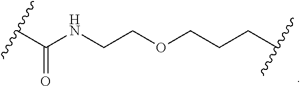 [SMILES: *CCCOCCNC(*)=O]

In some embodiments, L is

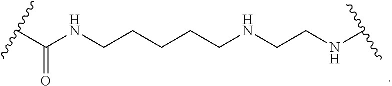 [SMILES: *NCCNCCCCCNC(*)=O]

In some embodiments, L is

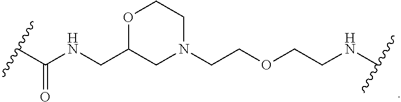 [SMILES: *NCCOCCN1CCOC(CNC(*)=O)C1]

In some embodiments, L is

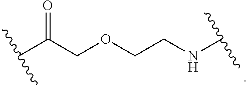 [SMILES: *NCCOCC(*)=O]

In some embodiments, L is

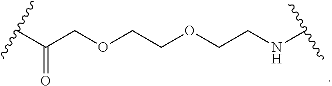 [SMILES: *NCCOCCOCC(*)=O]

In some embodiments, L is

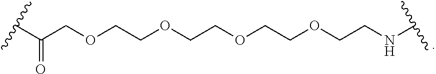 [SMILES: *NCCOCCOCCOCCOCC(*)=O]

In some embodiments, L is

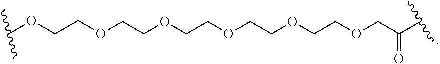 [SMILES: *OCCOCCOCCOCCOCCOCC(*)=O]

In some embodiments, L is

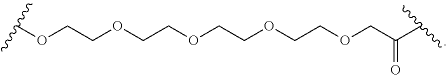 [SMILES: *OCCOCCOCCOCCOCC(*)=O]

In some embodiments, L is

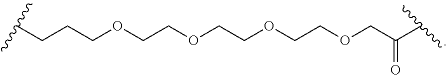 [SMILES: *CCCOCCOCCOCCOCC(*)=O]

In some embodiments, L is

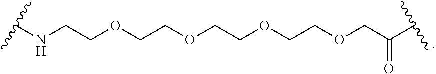 [SMILES: *NCCOCCOCCOCCOCC(*)=O]

In some embodiments, L is

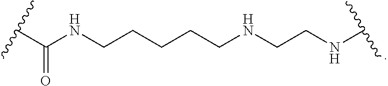 [SMILES: *NCCNCCCCCNC(*)=O]

In some embodiments, L is

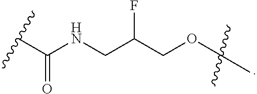 [SMILES: *OCC(F)CNC(*)=O]

In some embodiments, L is

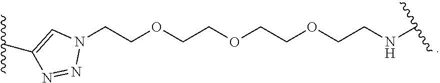 [SMILES: *NCCOCCOCCOCCn1cc(*)nn1]

In some embodiments, L is

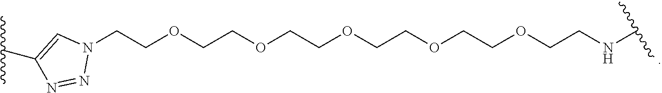 [SMILES: *NCCOCCOCCOCCOCCOCCn1cc(*)nn1]

In some embodiments, L is

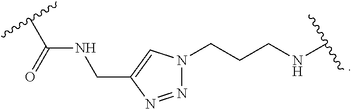 [SMILES: *NCCCn1cc(CNC(*)=O)nn1]

In some embodiments, L is

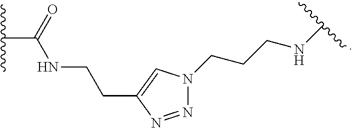 [SMILES: *NCCCn1cc(CCNC(*)=O)nn1]

In some embodiments, L is

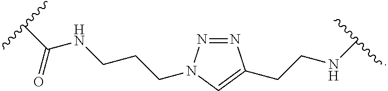 [SMILES: *NCCc1cn(CCCNC(*)=O)nn1]

In some embodiments, L is

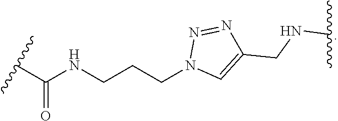 [SMILES: *NCc1cn(CCCNC(*)=O)nn1]

In some embodiments, L is

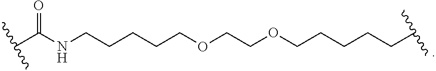 [SMILES: *CCCCCOCCOCCCCCNC(*)=O]

In some embodiments, L is

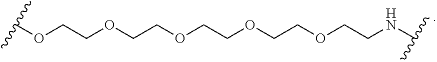 [SMILES: *NCCOCCOCCOCCOCCO*]

In some embodiments, L is

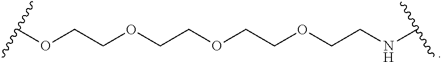 [SMILES: *NCCOCCOCCOCCO*]

In some embodiments, L is

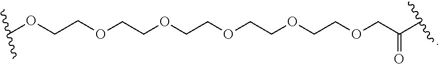 [SMILES: *OCCOCCOCCOCCOCCOCC(*)=O]

In some embodiments, L is

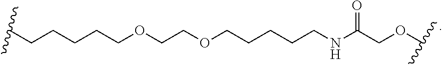 [SMILES: *CCCCCOCCOCCCCCNC(=O)CO*]

In some embodiments, L is

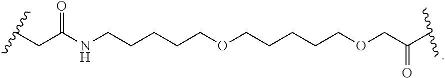 [SMILES: *CC(=O)NCCCCCOCCCCCOCC(*)=O]

In some embodiments, L is

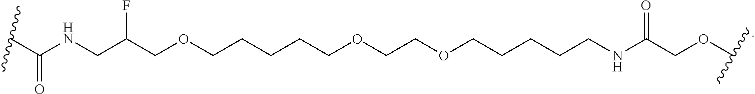 [SMILES: *OCC(=O)NCCCCCOCCOCCCCCOCC(F)CNC(*)=O]

In some embodiments, L is

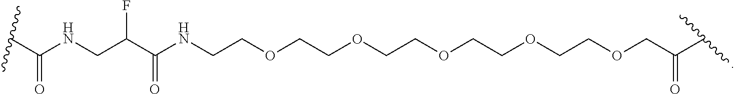 [SMILES: *C(=O)COCCOCCOCCOCCOCCNC(=O)C(F)CNC(*)=O]

In some embodiments, L is

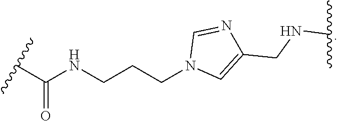 [SMILES: *NCc1cn(CCCNC(*)=O)cn1]

In some embodiments, L is

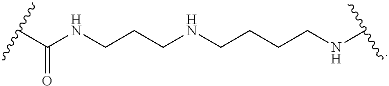 [SMILES: *NCCCCNCCCNC(*)=O]

In some embodiments, L is

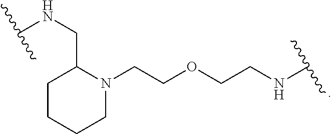 [SMILES: *NCCOCCN1CCCCC1CN*]

In some embodiments, L is

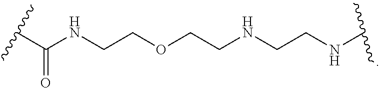 [SMILES: *NCCNCCOCCNC(*)=O]

In some embodiments, L is

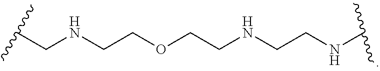 [SMILES: *CNCCOCCNCCN*]

In some embodiment, L is

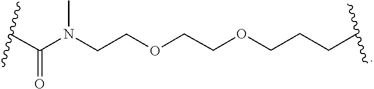 [SMILES: *CCCOCCOCCN(C)C(*)=O]

In some embodiment, L is

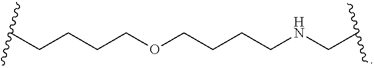 [SMILES: *CCCCOCCCCNC*]

In some embodiment, L is

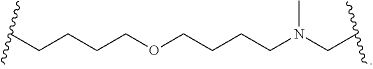 [SMILES: *CCCCOCCCCN(C)C*]

In some embodiments, L is

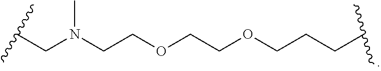 [SMILES: *CCCOCCOCCN(C)C*]

In some embodiments, L is

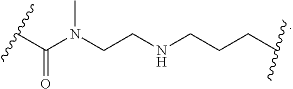 [SMILES: *CCCNCCN(C)C(*)=O]

In some embodiments, L is

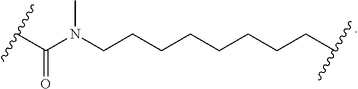 [SMILES: *CCCCCCCCN(C)C(*)=O]

In some embodiments, L is

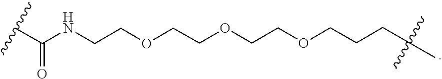 [SMILES: *CCCOCCOCCOCCNC(*)=O]

In some embodiments, L is

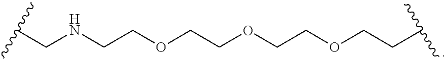 [SMILES: *CCOCCOCCOCCNC*]

In some embodiments, L is

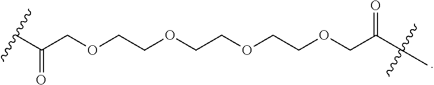 [SMILES: *C(=O)COCCOCCOCCOCC(*)=O]

In In some embodiments, L is

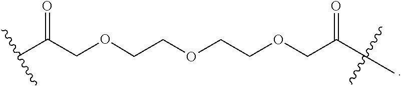 [SMILES: *C(=O)COCCOCCOCC(*)=O]

In some

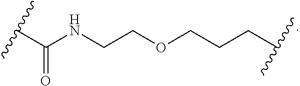 [SMILES: *CCCOCCNC(*)=O]

embodiments, L is

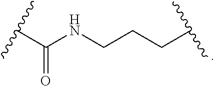 [SMILES: *CCCNC(*)=O]

In some embodiments, L is

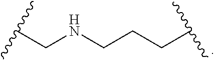 [SMILES: *CCCNC*]

In some embodiments, L is

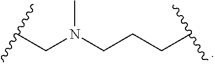 [SMILES: *CCCN(C)C*]

In some embodiments, L is

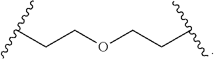 [SMILES: *CCOCC*]

In some embodiments, L is

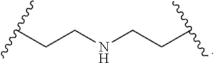 [SMILES: *CCNCC*]

In some embodiments, L is

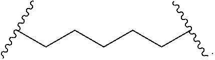 [SMILES: *CCCCC*]

In some embodiments, L is

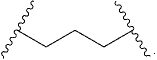 [SMILES: *CCC*]

In some embodiments, L is

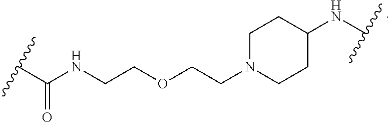 [SMILES: *NC1CCN(CCOCCNC(*)=O)CC1]

In some embodiments, L is

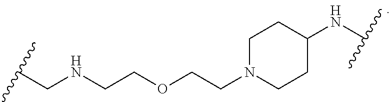 [SMILES: *CNCCOCCN1CCC(N*)CC1]

In some embodiments, L is

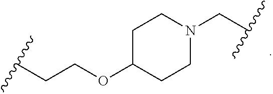 [SMILES: *CCOC1CCN(C*)CC1]

In some embodiments, L is

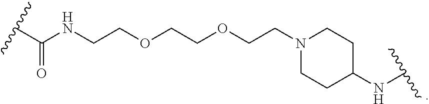 [SMILES: *NC1CCN(CCOCCOCCNC(*)=O)CC1]

In some embodiments, L is

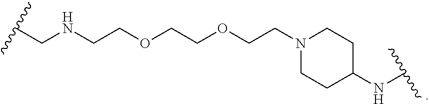 [SMILES: *CNCCOCCOCCN1CCC(N*)CC1]

In some embodiments, L is

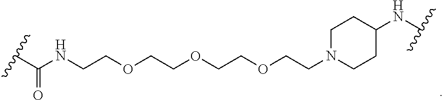 [SMILES: *NC1CCN(CCOCCOCCOCCNC(*)=O)CC1]

In some embodiments, L is

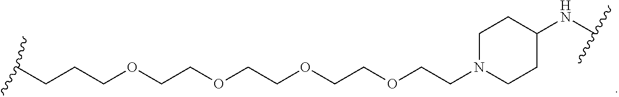 [SMILES: *CCCOCCOCCOCCOCCN1CCC(N*)CC1]

In some embodiments, L is

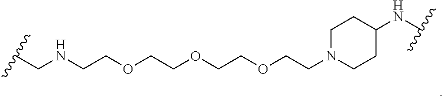 [SMILES: *CNCCOCCOCCOCCN1CCC(N*)CC1]

In some embodiments, L is

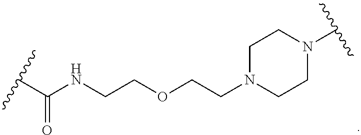 [SMILES: *C(=O)NCCOCCN1CCN(*)CC1]

In some embodiments, L is

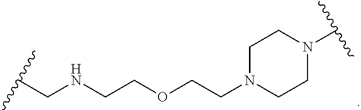 [SMILES: *CNCCOCCN1CCN(*)CC1]

In some embodiments, L is

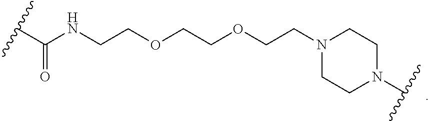 [SMILES: *C(=O)NCCOCCOCCN1CCN(*)CC1]

In some embodiments, L is

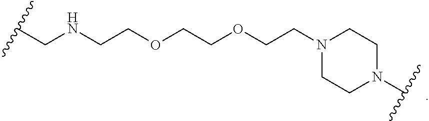 [SMILES: *CNCCOCCOCCN1CCN(*)CC1]

In some embodiments, L is

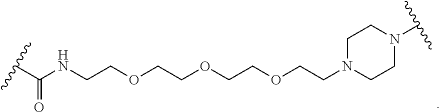 [SMILES: *C(=O)NCCOCCOCCOCCN1CCN(*)CC1]

In some embodiments, L is

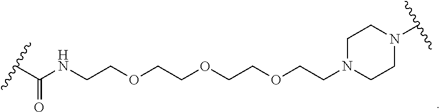 [SMILES: *C(=O)NCCOCCOCCOCCN1CCN(*)CC1]

In some embodiments, L is

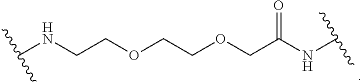 [SMILES: *NCCOCCOCC(=O)N*]

In some embodiments, L is

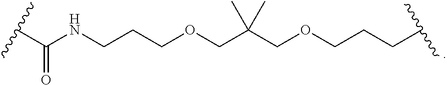 [SMILES: *CCCOCC(C)(C)COCCCNC(*)=O]

In some embodiments, L is

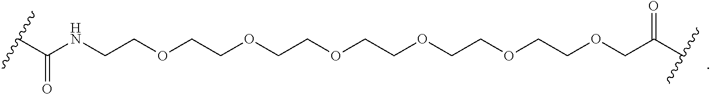 [SMILES: *C(=O)COCCOCCOCCOCCOCCOCCNC(*)=O]

In some embodiments, L is

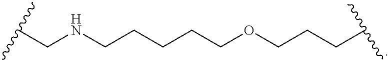 [SMILES: *CCCOCCCCCNC*]

In some embodiments, L is

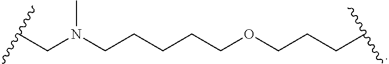 [SMILES: *CCCOCCCCCN(C)C*]

In some embodiments, L is

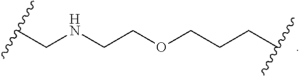 [SMILES: *CCCOCCNC*]

In some embodiments, L is

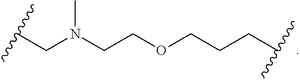 [SMILES: *CCCOCCN(C)C*]

In some embodiments, L is

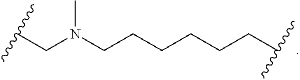 [SMILES: *CCCCCCN(C)C*]

In some embodiments, L is

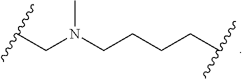 [SMILES: *CCCCN(C)C*]

In some embodiments, L is

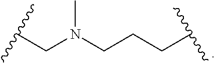 [SMILES: *CCCN(C)C*]

In some embodiments, L is

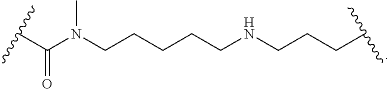 [SMILES: *CCCNCCCCCN(C)C(*)=O]

In some embodiments, L is

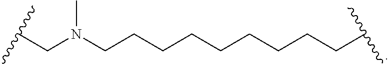 [SMILES: *CCCCCCCCCN(C)C*]

In some embodiments, L is

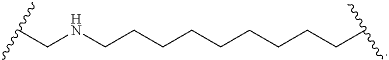 [SMILES: *CCCCCCCCCNC*]

In some embodiments, L is

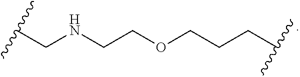 [SMILES: *CCCOCCNC*]

In some embodiments, L is

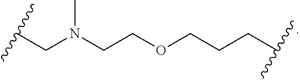 [SMILES: *CCCOCCN(C)C*]

In some embodiments, L is

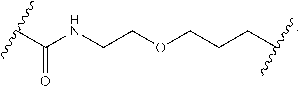 [SMILES: *CCCOCCNC(*)=O]

In some embodiments, L is

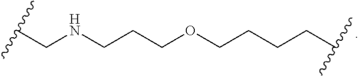 [SMILES: *CCCCOCCCNC*]

In some embodiments, L is

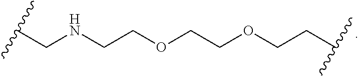 [SMILES: *CCOCCOCCNC*]

In some embodiments, L is

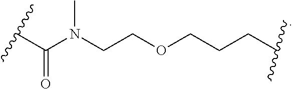 [SMILES: *CCCOCCN(C)C(*)=O]

In some embodiments, L is

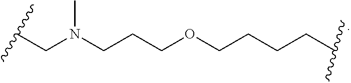 [SMILES: *CCCCOCCCN(C)C*]

In some embodiments, L is

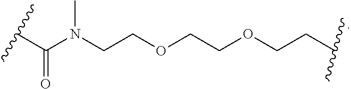 [SMILES: *CCOCCOCCN(C)C(*)=O]

In some embodiments, L is

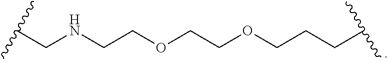 [SMILES: *CCCOCCOCCNC*]

In some embodiments, L is

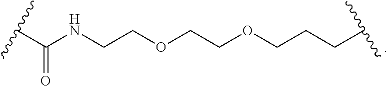 [SMILES: *CCCOCCOCCNC(*)=O]

In some embodiments, L is

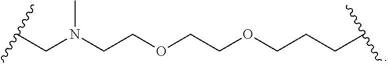 [SMILES: *CCCOCCOCCN(C)C*]

In some embodiments, L is

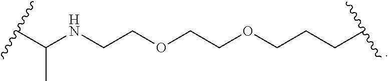 [SMILES: *CCCOCCOCCNC(*)C]

In some embodiments, L is

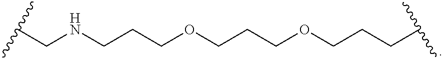 [SMILES: *CCCOCCCOCCCNC*]

In some embodiment, L is

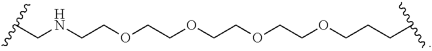 [SMILES: *CCCOCCOCCOCCOCCNC*]

In some embodiment, L is

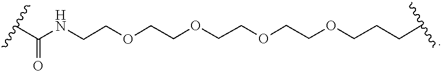 [SMILES: *CCCOCCOCCOCCOCCNC(*)=O]

In some embodiments, L is

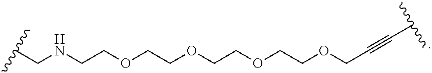 [SMILES: *C#CCOCCOCCOCCOCCNC*]

In some embodiments, L is

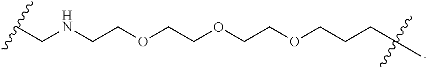 [SMILES: *CCCOCCOCCOCCNC*]

In some embodiments, L is

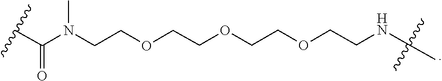 [SMILES: *NCCOCCOCCOCCN(C)C(*)=O]

In some embodiments, L is

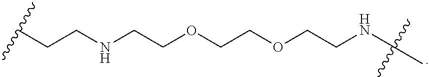 [SMILES: *CCNCCOCCOCCN*]

In some embodiments, L is

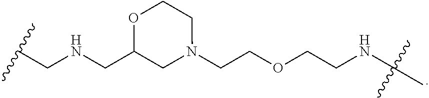 [SMILES: *CNCC1CN(CCOCCN*)CCO1]

In some embodiments, L is

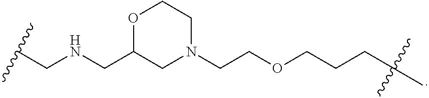 [SMILES: *CCCOCCN1CCOC(CNC*)C1]

In some embodiments, L is

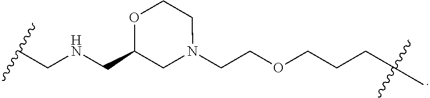 [SMILES: *CCCOCCN1CCO[C@H](CNC*)C1]

In some embodiments, L is

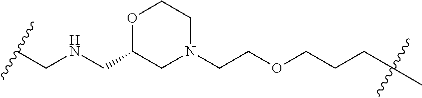 [SMILES: *CCCOCCN1CCO[C@@H](CNC*)C1]

In some embodiments, L is

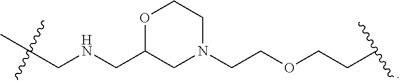 [SMILES: *CCOCCN1CCOC(CNC*)C1]

In some embodiments, L is

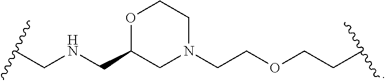 [SMILES: *CCOCCN1CCO[C@H](CNC*)C1]

In some embodiments, L is

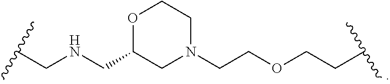 [SMILES: *CCOCCN1CCO[C@@H](CNC*)C1]

In some embodiments, L is

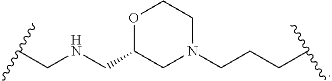 [SMILES: *CCCN1CCO[C@@H](CNC*)C1]

In some embodiments, L is

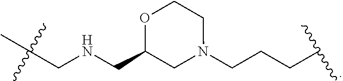 [SMILES: *CCCN1CCO[C@H](CNC*)C1]

In some embodiments, L is

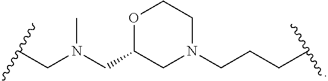 [SMILES: *CCCN1CCO[C@@H](CN(C)C*)C1]

In some embodiments, L is

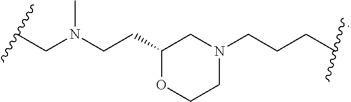 [SMILES: *CCCN1CCO[C@H](CCN(C)C*)C1]

In some embodiments, L is

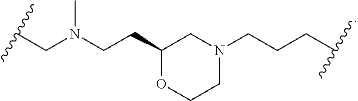 [SMILES: *CCCN1CCO[C@@H](CCN(C)C*)C1]

In some embodiments, L is

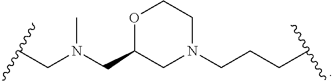 [SMILES: *CCCN1CCO[C@H](CN(C)C*)C1]

In some embodiments, L is

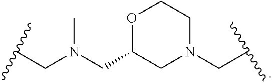 [SMILES: *CN(C)C[C@H]1CN(C*)CCO1]

In some embodiments, L is

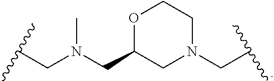 [SMILES: *CN(C)C[C@@H]1CN(C*)CCO1]

In some embodiments, L is

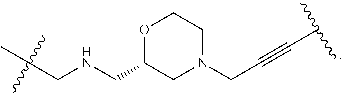 [SMILES: *C#CCN1CCO[C@@H](CNC*)C1]

In some embodiments, L is

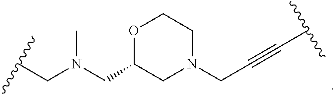 [SMILES: *C#CCN1CCO[C@@H](CN(C)C*)C1]

In some embodiments, L is

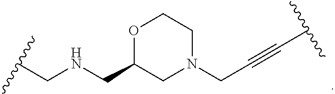 [SMILES: *C#CCN1CCO[C@H](CNC*)C1]

In some embodiments, L is

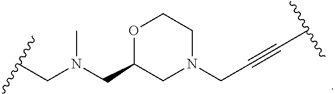 [SMILES: *C#CCN1CCO[C@H](CN(C)C*)C1]

In some embodiments, L is

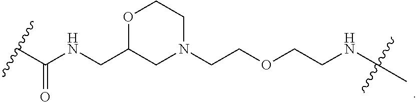 [SMILES: *NCCOCCN1CCOC(CNC(*)=O)C1]

In some embodiments, L is

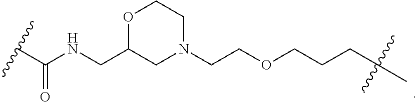 [SMILES: *CCCOCCN1CCOC(CNC(*)=O)C1]

In some embodiments, L is

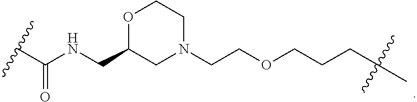 [SMILES: *CCCOCCN1CCO[C@H](CNC(*)=O)C1]

In some embodiments, L is

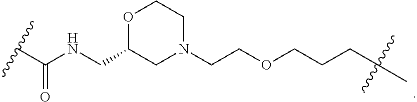 [SMILES: *CCCOCCN1CCO[C@@H](CNC(*)=O)C1]

In some embodiments, L is

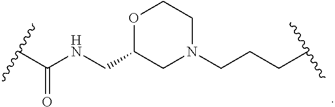 [SMILES: *CCCN1CCO[C@@H](CNC(*)=O)C1]

In some embodiments, L is

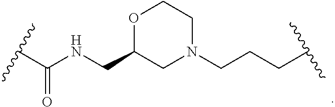 [SMILES: *CCCN1CCO[C@H](CNC(*)=O)C1]

In some embodiments, L is

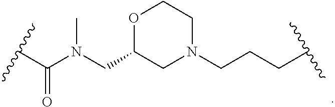 [SMILES: *CCCN1CCO[C@@H](CN(C)C(*)=O)C1]

In some embodiments, L is

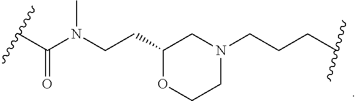 [SMILES: *CCCN1CCO[C@H](CCN(C)C(*)=O)C1]

In some embodiments, L is

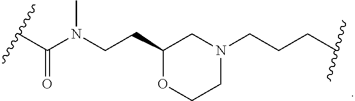 [SMILES: *CCCN1CCO[C@@H](CCN(C)C(*)=O)C1]

In some embodiments, L is

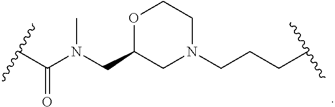 [SMILES: *CCCN1CCO[C@H](CN(C)C(*)=O)C1]

In some embodiments, L is

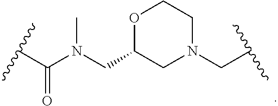 [SMILES: *CN1CCO[C@@H](CN(C)C(*)=O)C1]

In some embodiments, L is

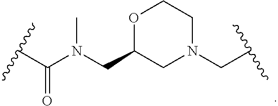 [SMILES: *CN1CCO[C@H](CN(C)C(*)=O)C1]

In some embodiments, L is

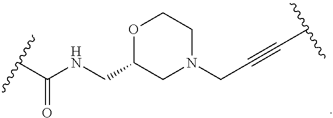 [SMILES: *C#CCN1CCO[C@@H](CNC(*)=O)C1]

In some embodiments, L is

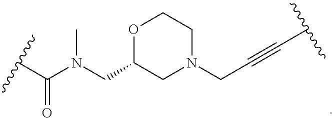 [SMILES: *C#CCN1CCO[C@@H](CN(C)C(*)=O)C1]

In some embodiments, L is

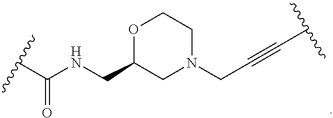 [SMILES: *C#CCN1CCO[C@H](CNC(*)=O)C1]

In some embodiments, L is

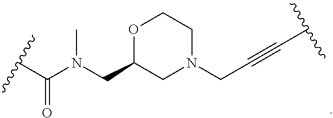 [SMILES: *C#CCN1CCO[C@H](CN(C)C(*)=O)C1]

In some embodiments, L is

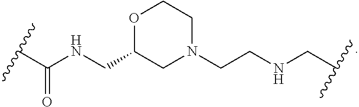 [SMILES: *CNCCN1CCO[C@@H](CNC(*)=O)C1]

In some embodiments, L is

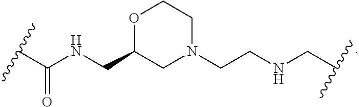 [SMILES: *CNCCN1CCO[C@H](CNC(*)=O)C1]

In some embodiments, L is

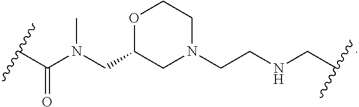 [SMILES: *CNCCN1CCO[C@@H](CN(C)C(*)=O)C1]

In some embodiments, L is

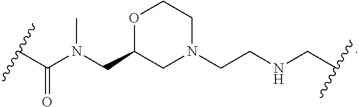 [SMILES: *CNCCN1CCO[C@H](CN(C)C(*)=O)C1]

In some embodiments, L is

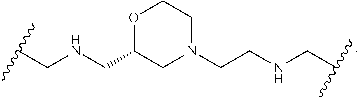 [SMILES: *CNCCN1CCO[C@@H](CNC*)C1]

In some embodiments, L is

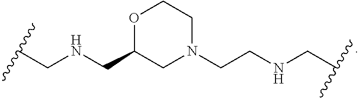 [SMILES: *CNCCN1CCO[C@H](CNC*)C1]

In some embodiments, L is

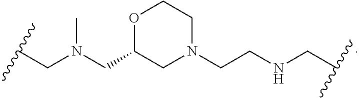 [SMILES: *CNCCN1CCO[C@@H](CN(C)C*)C1]

In some embodiments, L is

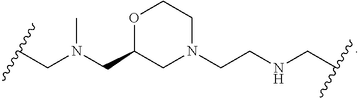 [SMILES: *CNCCN1CCO[C@H](CN(C)C*)C1]

In some embodiments, L is

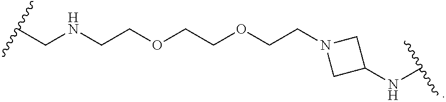 [SMILES: *CNCCOCCOCCN1CC(N*)C1]

In some embodiments, L is

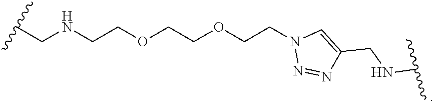 [SMILES: *CNCCOCCOCCn1cc(CN*)nn1]

In some embodiments, L is

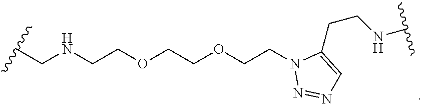 [SMILES: *CNCCOCCOCCn1nncc1CCN*]

In some embodiments, L is

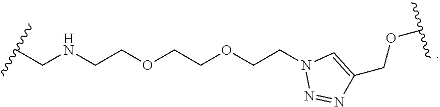 [SMILES: *CNCCOCCOCCn1cc(CO*)nn1]

In some embodiments, L is

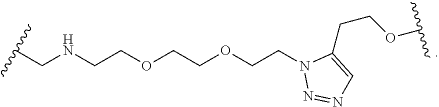 [SMILES: *CNCCOCCOCCn1nncc1CCO*]

In some embodiments, L is

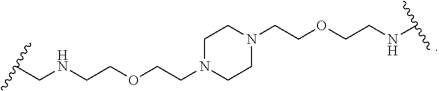 [SMILES: *CNCCOCCN1CCN(CCOCCN*)CC1]

In some embodiments, L is

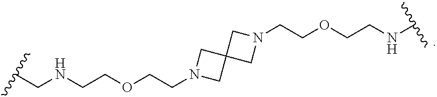 [SMILES: *CNCCOCCN1CC2(CN(CCOCCN*)C2)C1]

In some embodiments, L is

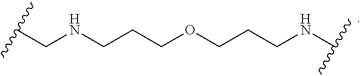 [SMILES: *CNCCCOCCCN*]

In some embodiments, L is

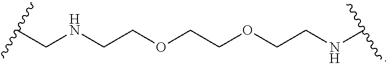 [SMILES: *CNCCOCCOCCN*]

In some embodiments, L is

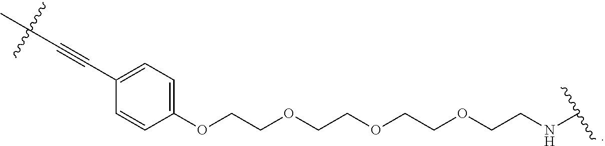 [SMILES: *C#Cc1ccc(OCCOCCOCCOCCN*)cc1]

In some embodiments, L is

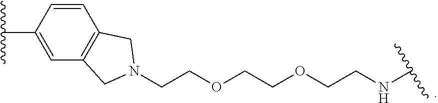 [SMILES: *NCCOCCOCCN1Cc2ccc(*)cc2C1]

In some embodiments, L is

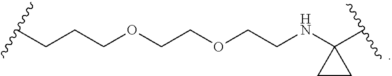 [SMILES: *CCCOCCOCCNC1(*)CC1]

In some embodiments, L is

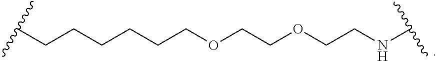 [SMILES: *CCCCCCOCCOCCN*]

In some embodiments, L is

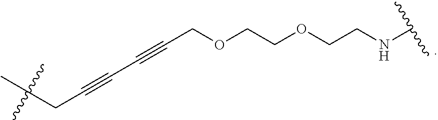 [SMILES: *CC#CC#CCOCCOCCN*]

In some embodiments, L is

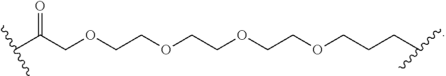 [SMILES: *CCCOCCOCCOCCOCC(*)=O]

In some embodiments, L is

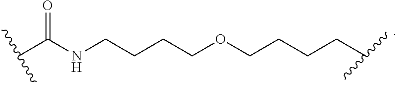 [SMILES: *CCCCOCCCCNC(*)=O]

In some embodiments, L is

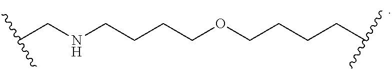 [SMILES: *CCCCOCCCCNC*]

In some embodiments, L is

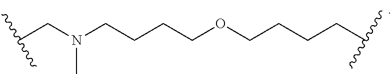 [SMILES: *CCCCOCCCCN(C)C*]

In some embodiments, L is

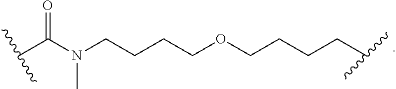 [SMILES: *CCCCOCCCCN(C)C(*)=O]

In some embodiments, L is

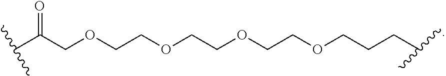 [SMILES: *CCCOCCOCCOCCOCC(*)=O]

In some embodiments, L is

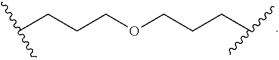 [SMILES: *CCCOCCC*]

In some embodiments, L is

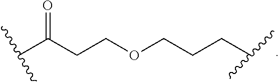 [SMILES: *CCCOCCC(*)=O]

In some embodiments, L is

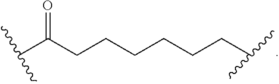 [SMILES: *CCCCCCC(*)=O]

In some embodiments, L is

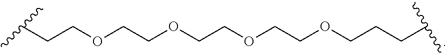 [SMILES: *CCCOCCOCCOCCOCC*]

In some embodiments, L is

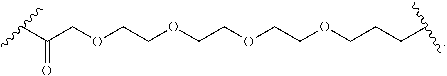 [SMILES: *CCCOCCOCCOCCOCC(*)=O]

In some embodiments, L is

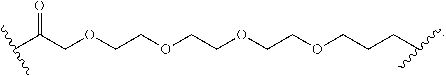 [SMILES: *CCCOCCOCCOCCOCC(*)=O]

In some embodiments, L is

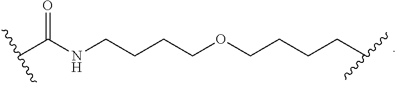 [SMILES: *CCCCOCCCCNC(*)=O]

In some embodiments, L is

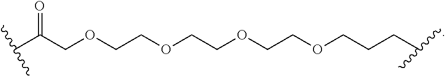 [SMILES: *CCCOCCOCCOCCOCC(*)=O]

In some embodiments, L is

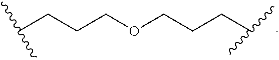 [SMILES: *CCCOCCC*]

In some embodiments, L is

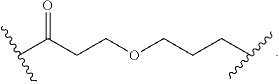 [SMILES: *CCCOCCC(*)=O]

In some embodiments, L is

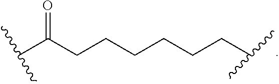 [SMILES: *CCCCCCC(*)=O]

In some embodiments, L is

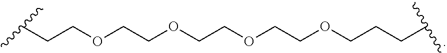 [SMILES: *CCCOCCOCCOCCOCC*]

In some embodiments, L is

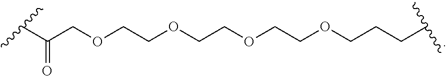 [SMILES: *CCCOCCOCCOCCOCC(*)=O]

In some embodiments, L is

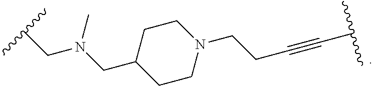 [SMILES: *C#CCCN1CCC(CN(C)C*)CC1]

In some embodiments, L is

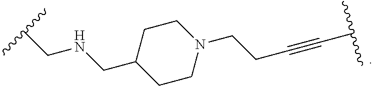 [SMILES: *C#CCCN1CCC(CNC*)CC1]

In some embodiments, L is

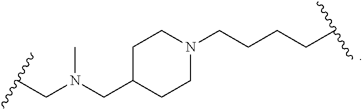 [SMILES: *CCCCN1CCC(CN(C)C*)CC1]

In some embodiments, L is

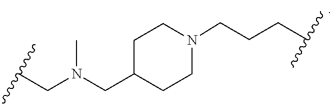 [SMILES: *CCCN1CCC(CN(C)C*)CC1]

In some embodiments, L is

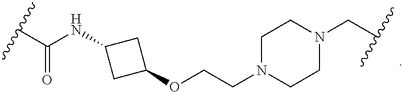 [SMILES: *CN1CCN(CCO[C@H]2C[C@H](NC(*)=O)C2)CC1]

In some embodiments, L is

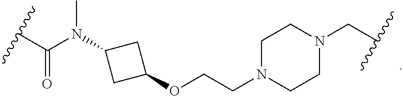 [SMILES: *CN1CCN(CCO[C@H]2C[C@H](N(C)C(*)=O)C2)CC1]

In some embodiments, L is

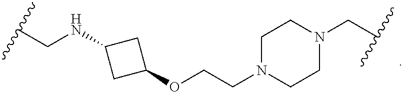 [SMILES: *CN[C@H]1C[C@H](OCCN2CCN(C*)CC2)C1]

In some embodiments, L is

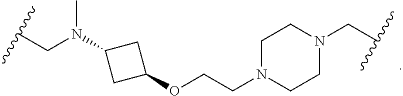 [SMILES: *CN1CCN(CCO[C@H]2C[C@H](N(C)C*)C2)CC1]

In some embodiments, L is

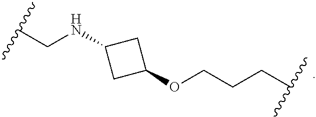 [SMILES: *CCCO[C@H]1C[C@H](NC*)C1]

In some embodiments, L is

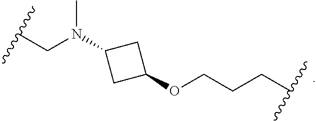 [SMILES: *CCCO[C@H]1C[C@H](N(C)C*)C1]

In some embodiments, L is

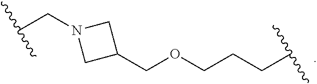 [SMILES: *CCCOCC1CN(C*)C1]

In some

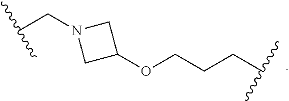 [SMILES: *CCCOC1CN(C*)C1]

In some embodiments, L is embodiments, L is

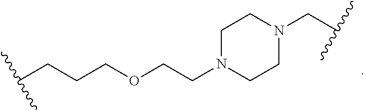 [SMILES: *CCCOCCN1CCN(C*)CC1]

In some embodiments, L is

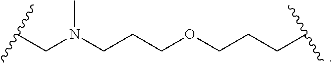 [SMILES: *CCCOCCCN(C)C*]

In some embodiments, L is

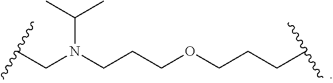 [SMILES: *CCCOCCCN(C*)C(C)C]

In some embodiments, is

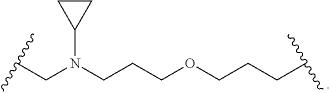 [SMILES: *CCCOCCCN(C*)C1CC1]

In some embodiments, L is

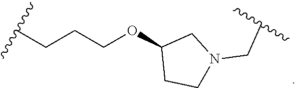 [SMILES: *CCCO[C@@H]1CCN(C*)C1]

In some embodiments, L is

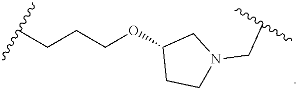 [SMILES: *CCCO[C@H]1CCN(C*)C1]

In some embodiments, L is

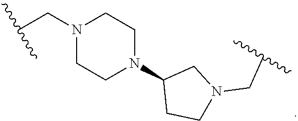 [SMILES: *CN1CCN([C@@H]2CCN(C*)C2)CC1]

In some embodiments, L is

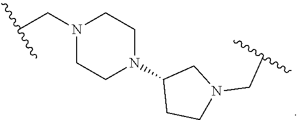 [SMILES: *CN1CCN([C@H]2CCN(C*)C2)CC1]

In some embodiments, L is

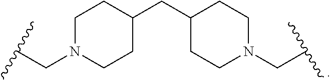 [SMILES: *CN1CCC(CC2CCN(C*)CC2)CC1]

In some embodiments, L is

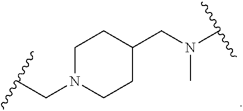 [SMILES: *CN1CCC(CN(*)C)CC1]

In some embodiments, L is

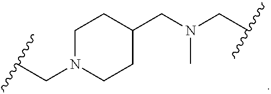 [SMILES: *CN(C)CC1CCN(C*)CC1]

In some embodiments, L is

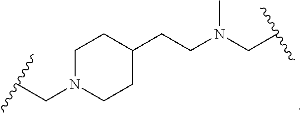 [SMILES: *CN(C)CCC1CCN(C*)CC1]

In some embodiments, L is

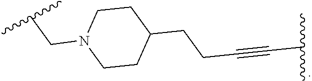 [SMILES: *C#CCCC1CCN(C*)CC1]

In some embodiments, L is

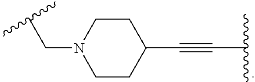 [SMILES: *C#CC1CCN(C*)CC1]

In some embodiments, L is

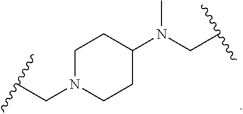 [SMILES: *CN1CCC(N(C)C*)CC1]

In some embodiments, L is

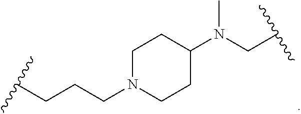 [SMILES: *CCCN1CCC(N(C)C*)CC1]

In some embodiments, L is

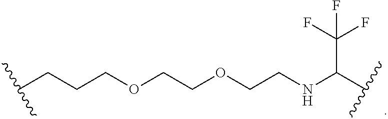 [SMILES: *CCCOCCOCCNC(*)C(F)(F)F]

In some embodiments, L is

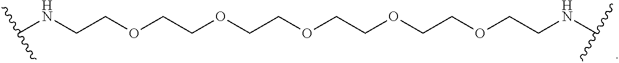 [SMILES: *NCCOCCOCCOCCOCCOCCN*]

In some embodiments, L is

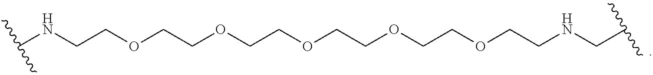 [SMILES: *CNCCOCCOCCOCCOCCOCCN*]

In some embodiments, L is

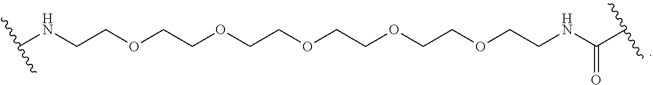 [SMILES: *NCCOCCOCCOCCOCCOCCNC(*)=O]

In some embodiments, L is

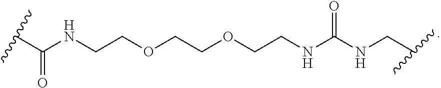 [SMILES: *CNC(=O)NCCOCCOCCNC(*)=O]

In some embodiments, L is

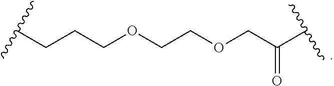 [SMILES: *CCCOCCOCC(*)=O]

In some embodiments, L is

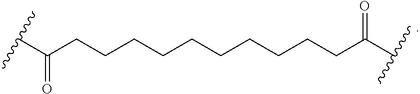 [SMILES: *C(=O)CCCCCCCCCCC(*)=O]

In some embodiments, L is

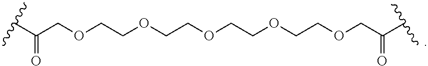 [SMILES: *C(=O)COCCOCCOCCOCCOCC(*)=O]

In some embodiments, L is

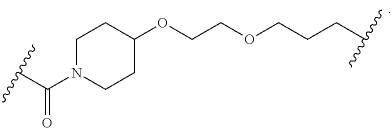 [SMILES: *CCCOCCOC1CCN(C(*)=O)CC1]

In some embodiments, L is

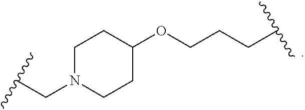 [SMILES: *CCCOC1CCN(C*)CC1]

In some embodiments, L is

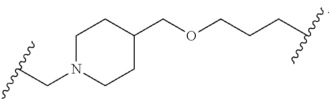 [SMILES: *CCCOCC1CCN(C*)CC1]

In some embodiments, L is

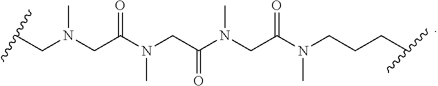 [SMILES: *CCCN(C)C(=O)CN(C)C(=O)CN(C)C(=O)CN(C)C*]

In some embodiments, L is

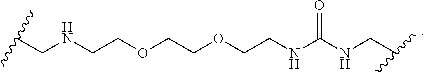 [SMILES: *CNCCOCCOCCNC(=O)NC*]

In some embodiment, L is

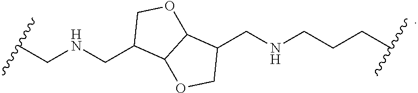 [SMILES: *CCCNCC1COC2C(CNC*)COC12]

In some embodiments, L is

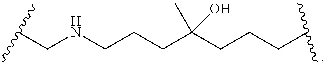 [SMILES: *CCCC(C)(O)CCCNC*]

In some embodiments, L is

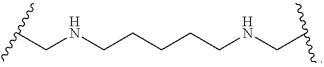 [SMILES: *CNCCCCCNC*]

In some embodiments, L is

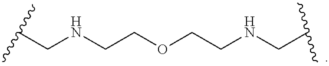 [SMILES: *CNCCOCCNC*]

In some embodiments, L is

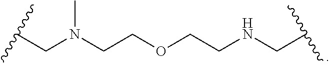 [SMILES: *CNCCOCCN(C)C*]

In some embodiments, L is

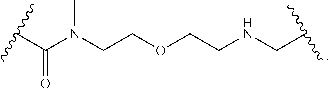 [SMILES: *CNCCOCCN(C)C(*)=O]

In some embodiments, L is

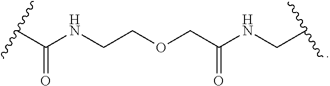 [SMILES: *CNC(=O)COCCNC(*)=O]

In some embodiments, L is

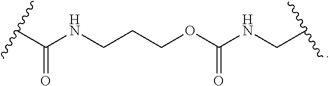 [SMILES: *CNC(=O)OCCCNC(*)=O]

In some embodiments, L is

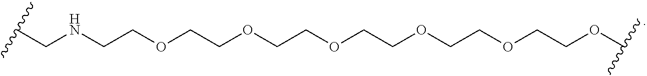 [SMILES: *CNCCOCCOCCOCCOCCOCCO*]

In some embodiments, L is

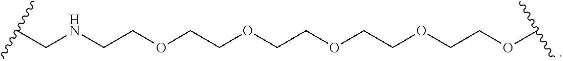 [SMILES: *CNCCOCCOCCOCCOCCO*]

In some embodiments, L is

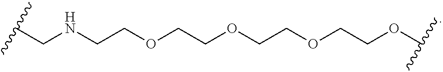 [SMILES: *CNCCOCCOCCOCCO*]

In some embodiments, L is

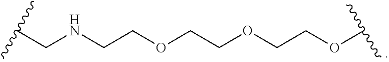 [SMILES: *CNCCOCCOCCO*]

In some embodiments, L is

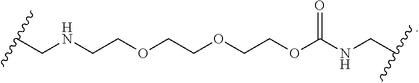 [SMILES: *CNCCOCCOCCOC(=O)NC*]

In some embodiments, L is

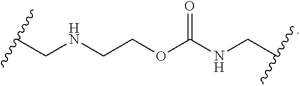 [SMILES: *CNCCOC(=O)NC*]

In some embodiments, L is

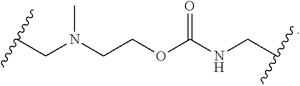 [SMILES: *CNC(=O)OCCN(C)C*]

In some embodiments, L is

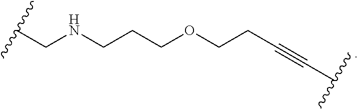 [SMILES: *C#CCCOCCCNC*]

In some embodiments, L is

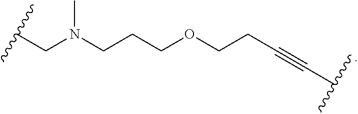 [SMILES: *C#CCCOCCCN(C)C*]

In some embodiments, L is

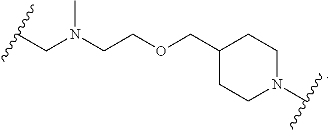 [SMILES: *CN(C)CCOCC1CCN(*)CC1]

In some embodiments, L is

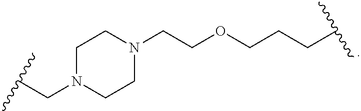 [SMILES: *CCCOCCN1CCN(C*)CC1]

In some embodiments, L is

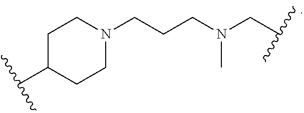 [SMILES: *CN(C)CCCN1CCC(*)CC1]

In some embodiments, L is

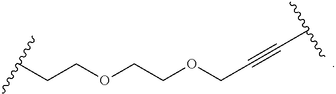 [SMILES: *C#CCOCCOCC*]

In some embodiments, is

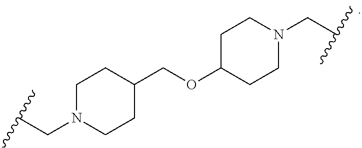 [SMILES: *CN1CCC(COC2CCN(C*)CC2)CC1]

In some embodiments, L is

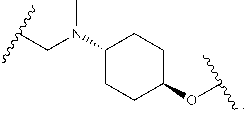 [SMILES: *CN(C)[C@H]1CC[C@H](O*)CC1]

In some embodiments, L is

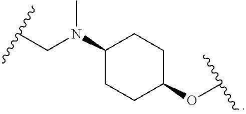 [SMILES: *CN(C)[C@H]1CC[C@@H](O*)CC1]

In some embodiments, L is

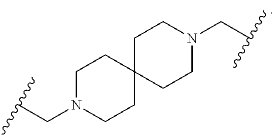 [SMILES: *CN1CCC2(CC1)CCN(C*)CC2]

In some embodiments, L is

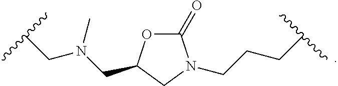 [SMILES: *CCCN1C[C@@H](CN(C)C*)OC1=O]

In some embodiments, L is

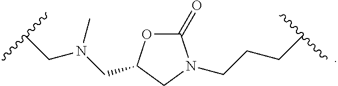 [SMILES: *CCCN1C[C@H](CN(C)C*)OC1=O]

In some embodiments, L is

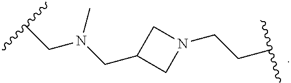 [SMILES: *CCN1CC(CN(C)C*)C1]

In some embodiments, L is

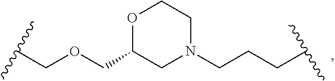 [SMILES: *CCCN1CCO[C@@H](COC*)C1]

In some embodiments, L is

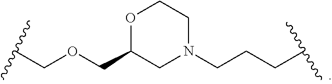 [SMILES: *CCCN1CCO[C@H](COC*)C1]

In some embodiments, L is

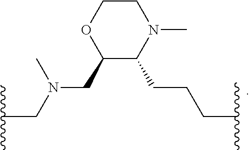 [SMILES: *CCC[C@@H]1[C@@H](CN(C)C*)OCCN1C]

In some embodiments, L is

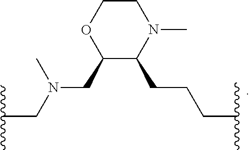 [SMILES: *CCC[C@H]1[C@@H](CN(C)C*)OCCN1C]

In some embodiments, L is

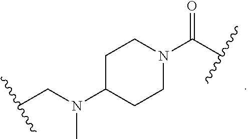 [SMILES: *CN(C)C1CCN(C(*)=O)CC1]

In some embodiments, L is

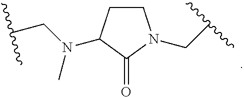 [SMILES: *CN1CCC(N(C)C*)C1=O]

In some embodiments, L is

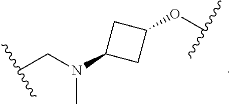 [SMILES: *CN(C)[C@H]1C[C@H](O*)C1]

In some embodiments, L is

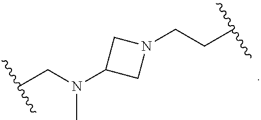 [SMILES: *CCN1CC(N(C)C*)C1]

In some embodiments, L is

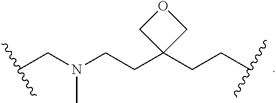 [SMILES: *CCC1(CCN(C)C*)COC1]

In some embodiments, L is

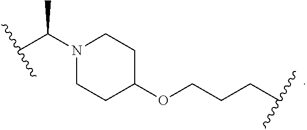 [SMILES: *CCCOC1CCN([C@@H](*)C)CC1]

In some embodiments, L is

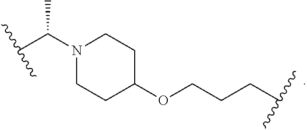 [SMILES: *CCCOC1CCN([C@H](*)C)CC1]

In some embodiments, L is

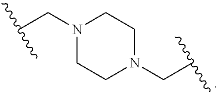 [SMILES: *CN1CCN(C*)CC1]

In some embodiments, L is

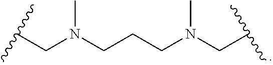 [SMILES: *CN(C)CCCN(C)C*]

In some embodiments, L is

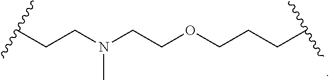 [SMILES: *CCCOCCN(C)CC*]

In some embodiments, L is

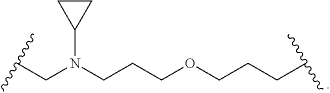 [SMILES: *CCCOCCCN(C*)C1CC1]

In some embodiments, L is

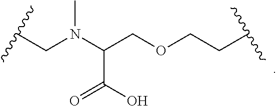 [SMILES: *CCOCC(C(=O)O)N(C)C*]

In some embodiments, L is

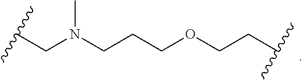 [SMILES: *CCOCCCN(C)C*]

In some embodiments, L is

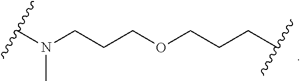 [SMILES: *CCCOCCCN(*)C]

In some embodiments, L is

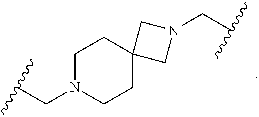 [SMILES: *CN1CCC2(CC1)CN(C*)C2]

In some embodiments, L is

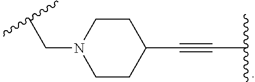 [SMILES: *C#CC1CCN(C*)CC1]

In some embodiments, L is

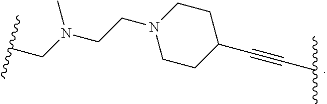 [SMILES: *C#CC1CCN(CCN(C)C*)CC1]

In some embodiments, L is

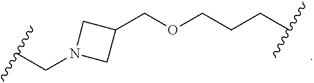 [SMILES: *CCCOCC1CN(C*)C1]

In some embodiments, L is a covalent bond. In some embodiments, L is

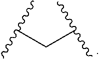 [SMILES: *C*]

In some embodiments, L is

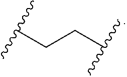 [SMILES: *CC*]

In some embodiments, L is

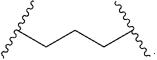 [SMILES: *CCC*]

In some embodiments, L is

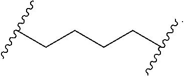 [SMILES: *CCCC*]

In some embodiments, L is

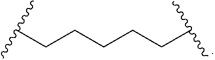 [SMILES: *CCCCC*]

In some embodiments, L is

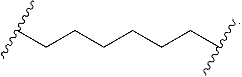 [SMILES: *CCCCCC*]

In some embodiments, L is

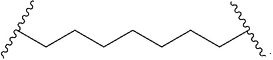 [SMILES: *CCCCCCC*]

In some embodiments, L is

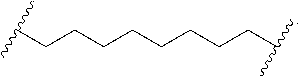 [SMILES: *CCCCCCCC*]

In some embodiments, L is

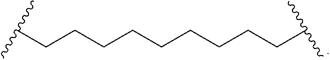 [SMILES: *CCCCCCCCC*]

In some embodiments, L is a covalent bond. In some embodiments, L is

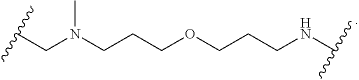 [SMILES: *CN(C)CCCOCCCN*]

In some embodiments, L is

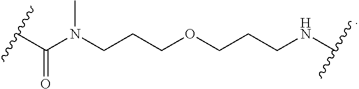 [SMILES: *NCCCOCCCN(C)C(*)=O]

In some embodiments, L is

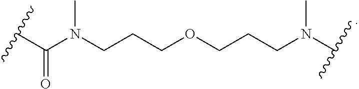 [SMILES: *C(=O)N(C)CCCOCCCN(*)C]

In some embodiments, L is

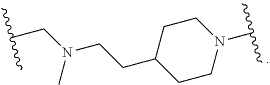 [SMILES: *CN(C)CCC1CCN(*)CC1]

In some embodiments, L is

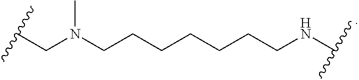 [SMILES: *CN(C)CCCCCCCN*]

In some embodiments, L is

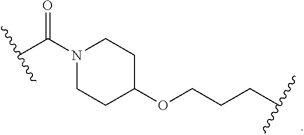 [SMILES: *CCCOC1CCN(C(*)=O)CC1]

In some embodiments, L is

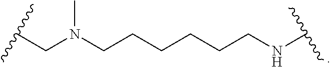 [SMILES: *CN(C)CCCCCCN*]

In some embodiments, L is

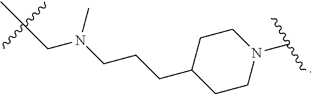 [SMILES: *CN(C)CCCC1CCN(*)CC1]

In some embodiments, L is

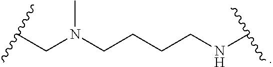 [SMILES: *CN(C)CCCCN*]

In some embodiments, L is

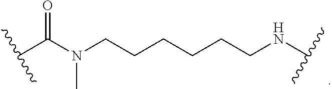 [SMILES: *NCCCCCCN(C)C(*)=O]

In some embodiments, L is

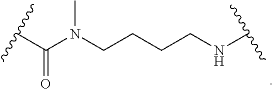 [SMILES: *NCCCCN(C)C(*)=O]

In some embodiments, L is

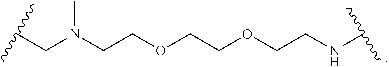 [SMILES: *CN(C)CCOCCOCCN*]

In some embodiments, L is

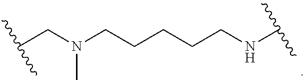 [SMILES: *CN(C)CCCCCN*]

In some embodiments, L is

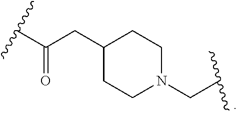 [SMILES: *CN1CCC(CC(*)=O)CC1]

In some embodiments,

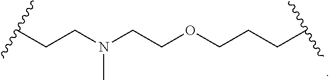 [SMILES: *CCCOCCN(C)CC*]

In some L is

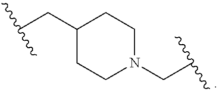 [SMILES: *CC1CCN(C*)CC1]

In some embodiments, L is embodiments, L is

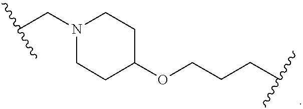 [SMILES: *CCCOC1CCN(C*)CC1]

In some embodiments, L is

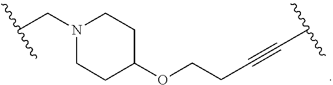 [SMILES: *C#CCCOC1CCN(C*)CC1]

In some embodiments, L is

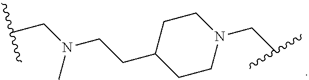 [SMILES: *CN(C)CCC1CCN(C*)CC1]

In some embodiments, L is

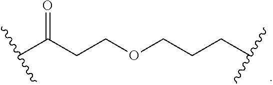 [SMILES: *CCCOCCC(*)=O]

In some embodiments, L is

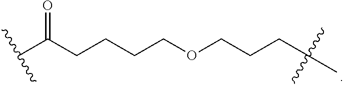 [SMILES: *CCCOCCCCC(*)=O]

In some embodiments, L is

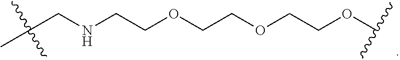 [SMILES: *CNCCOCCOCCO*]

In some embodiments, L is

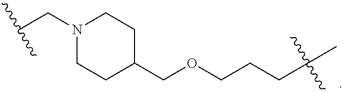 [SMILES: *CCCOCC1CCN(C*)CC1]

In some embodiments, L is

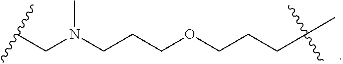 [SMILES: *CCCOCCCN(C)C*]

In some embodiments, L is

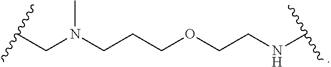 [SMILES: *CN(C)CCCOCCN*]

In some embodiments, L is

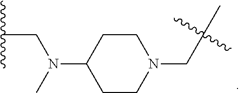 [SMILES: *CN1CCC(N(C)C*)CC1]

In some embodiments, L is

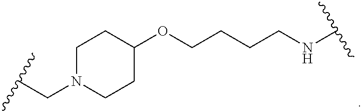 [SMILES: *CN1CCC(OCCCCN*)CC1]

In some embodiments, L is

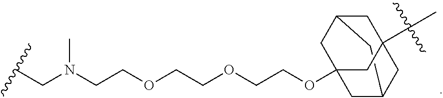 [SMILES: *CN(C)CCOCCOCCOC12CC3CC(CC(*)(C3)C1)C2]

In some embodiments, L is

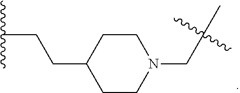 [SMILES: *CCC1CCN(C*)CC1]

In some embodiments, L is

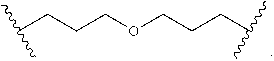 [SMILES: *CCCOCCC*]

In some embodiments, L is

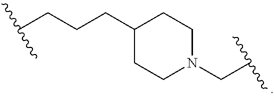 [SMILES: *CCCC1CCN(C*)CC1]

In some embodiments, L is

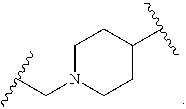 [SMILES: *CN1CCC(*)CC1]

In some embodiments, L is

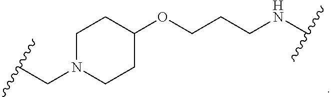 [SMILES: *CN1CCC(OCCCN*)CC1]

In some embodiments, L is

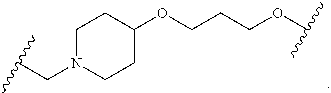 [SMILES: *CN1CCC(OCCCO*)CC1]

In some embodiments, L is

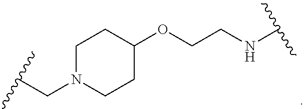 [SMILES: *CN1CCC(OCCN*)CC1]

In some embodiments, L is

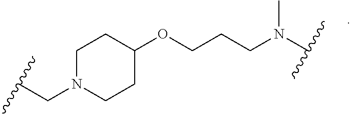 [SMILES: *CN1CCC(OCCCN(*)C)CC1]

In some embodiments, L is

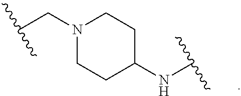 [SMILES: *CN1CCC(N*)CC1]

In some embodiments, L is

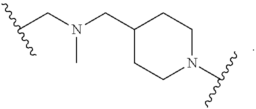 [SMILES: *CN(C)CC1CCN(*)CC1]

In some embodiments, L is

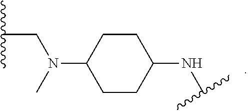 [SMILES: *CN(C)C1CCC(N*)CC1]

In some embodiments, L is

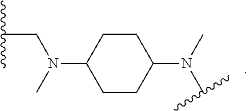 [SMILES: *CN(C)C1CCC(N(*)C)CC1]

In some embodiments, L is

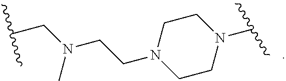 [SMILES: *CN(C)CCN1CCN(*)CC1]

In some embodiments, L is

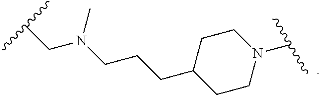 [SMILES: *CN(C)CCCC1CCN(*)CC1]

In some embodiments, L is

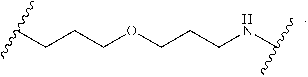 [SMILES: *CCCOCCCN*]

In some embodiments, L is

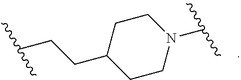 [SMILES: *CCC1CCN(*)CC1]

In some embodiments, L is

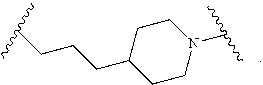 [SMILES: *CCCC1CCN(*)CC1]

In some embodiments, L is

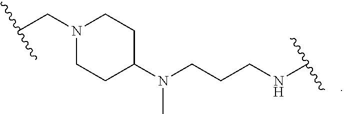 [SMILES: *CN1CCC(N(C)CCCN*)CC1]

In some embodiments, L is

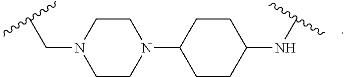 [SMILES: *CN1CCN(C2CCC(N*)CC2)CC1]

In some embodiments, L is

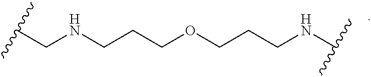 [SMILES: *CNCCCOCCCN*]

In some embodiments, L is

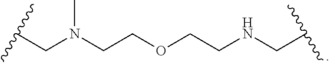 [SMILES: *CNCCOCCN(C)C*]

In some embodiments, L is

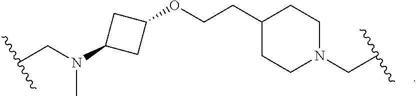 [SMILES: *CN1CCC(CCO[C@H]2C[C@H](N(C)C*)C2)CC1]

In some embodiments, L is

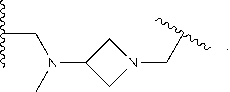 [SMILES: *CN1CC(N(C)C*)C1]

In some embodiments, L is

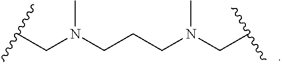 [SMILES: *CN(C)CCCN(C)C*]

In some embodiments, L is

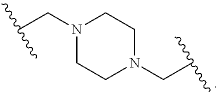 [SMILES: *CN1CCN(C*)CC1]

In some embodiments, L is

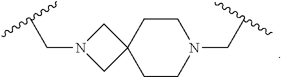 [SMILES: *CN1CCC2(CC1)CN(C*)C2]

In some embodiments, L is

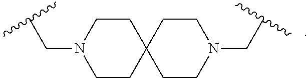 [SMILES: *CN1CCC2(CC1)CCN(C*)CC2]

In some embodiments, L is

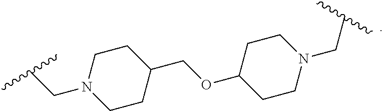 [SMILES: *CN1CCC(COC2CCN(C*)CC2)CC1]

In some embodiments, L is

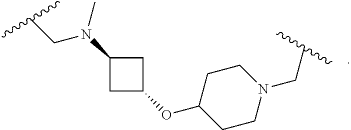 [SMILES: *CN1CCC(O[C@H]2C[C@H](N(C)C*)C2)CC1]

In some embodiments, L is In some embodiments, L is

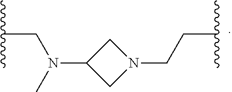 [SMILES: *CCN1CC(N(C)C*)C1]

In some embodiments, Lis

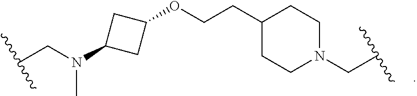 [SMILES: *CN1CCC(CCO[C@H]2C[C@H](N(C)C*)C2)CC1]

In some embodiments, L is

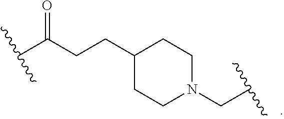 [SMILES: *CN1CCC(CCC(*)=O)CC1]

In some embodiments, L is

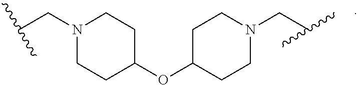 [SMILES: *CN1CCC(OC2CCN(C*)CC2)CC1]

In some embodiments, L is

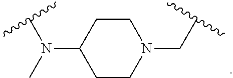 [SMILES: *CN1CCC(N(*)C)CC1]

In some embodiments, L is

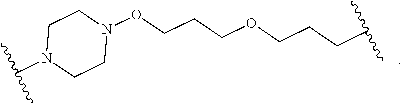 [SMILES: *CCCOCCCON1CCN(*)CC1]

In In some embodiments, L is

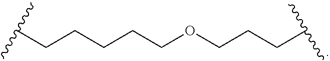 [SMILES: *CCCCCOCCC*]

In some embodiments, L is

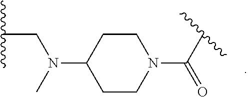 [SMILES: *CN(C)C1CCN(C(*)=O)CC1]

In some embodiments, L is

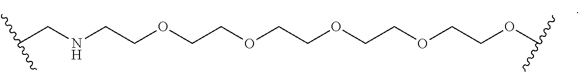 [SMILES: *CNCCOCCOCCOCCOCCO*]

In some embodiments, L is

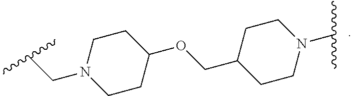 [SMILES: *CN1CCC(OCC2CCN(*)CC2)CC1]

In some embodiments, L is

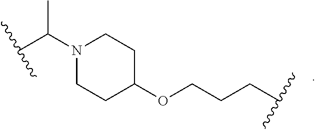 [SMILES: *CCCOC1CCN(C(*)C)CC1]

In some embodiments, L is

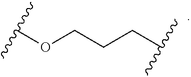 [SMILES: *CCCO*]

In some embodiments, L is

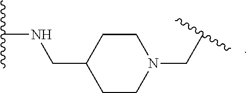 [SMILES: *CN1CCC(CN*)CC1]

In some embodiments, L

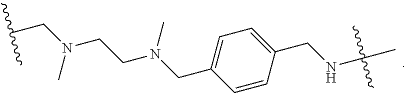 [SMILES: *CN(C)CCN(C)Cc1ccc(CN*)cc1]

In some embodiments, L is

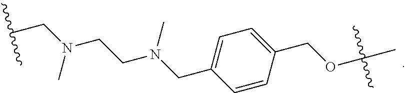 [SMILES: *CN(C)CCN(C)Cc1ccc(CO*)cc1]

In some embodiments, L is

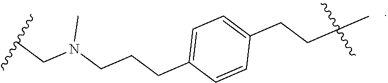 [SMILES: *CCc1ccc(CCCN(C)C*)cc1]

In some embodiments, L is

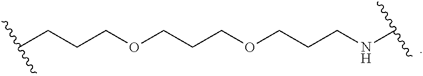 [SMILES: *CCCOCCCOCCCN*]

In some embodiments, L is

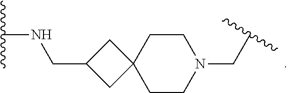 [SMILES: *CN1CCC2(CC1)CC(CN*)C2]

In some embodiments, L is

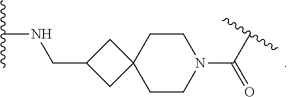 [SMILES: *NCC1CC2(CCN(C(*)=O)CC2)C1]

In some embodiments, L is

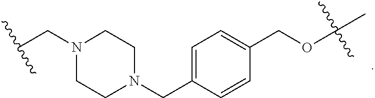 [SMILES: *CN1CCN(Cc2ccc(CO*)cc2)CC1]

In some embodiments, L is

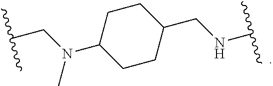 [SMILES: *CN(C)C1CCC(CN*)CC1]

In some embodiments, L is

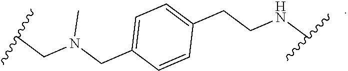 [SMILES: *CN(C)Cc1ccc(CCN*)cc1]

In some embodiments, L is

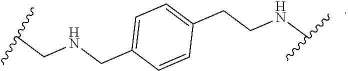 [SMILES: *CNCc1ccc(CCN*)cc1]

In some embodiments, L is

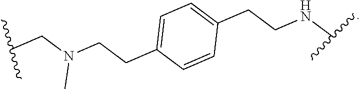 [SMILES: *CN(C)CCc1ccc(CCN*)cc1]

In some embodiments, L is

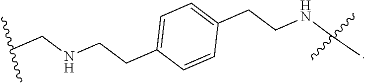 [SMILES: *CNCCc1ccc(CCN*)cc1]

In some embodiments, L is

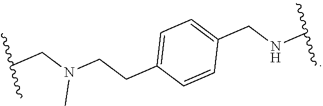 [SMILES: *CN(C)CCc1ccc(CN*)cc1]

In some embodiments, L is

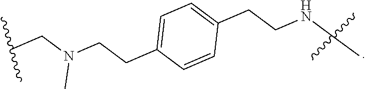 [SMILES: *CN(C)CCc1ccc(CCN*)cc1]

In some embodiments, L is

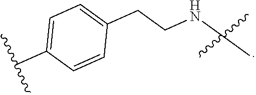 [SMILES: *NCCc1ccc(*)cc1]

In In some embodiments, L is

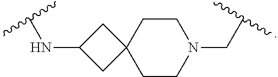 [SMILES: *CN1CCC2(CC1)CC(N*)C2]

In some In In some embodiments, L is

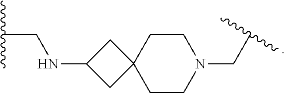 [SMILES: *CNC1CC2(CCN(C*)CC2)C1]

embodiments, L is

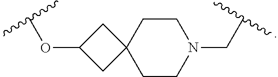 [SMILES: *CN1CCC2(CC1)CC(O*)C2]

In some embodiments, L is

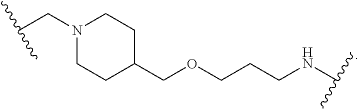 [SMILES: *CN1CCC(COCCCN*)CC1]

In some embodiments, L is

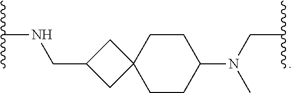 [SMILES: *CN(C)C1CCC2(CC1)CC(CN*)C2]

In some embodiments, L is

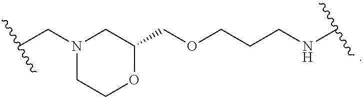 [SMILES: *CN1CCO[C@@H](COCCCN*)C1]

In some embodiments, L is

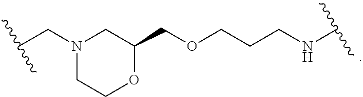 [SMILES: *CN1CCO[C@H](COCCCN*)C1]

In some embodiments, L is

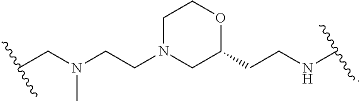 [SMILES: *CN(C)CCN1CCO[C@H](CCN*)C1]

In some embodiments, L is

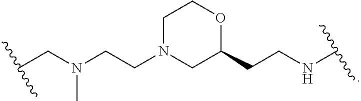 [SMILES: *CN(C)CCN1CCO[C@@H](CCN*)C1]

In some embodiments, L is

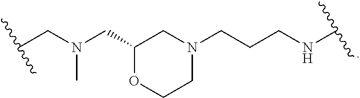 [SMILES: *CN(C)C[C@@H]1CN(CCCN*)CCO1]

In some embodiments, L is

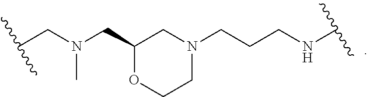 [SMILES: *CN(C)C[C@H]1CN(CCCN*)CCO1]

In some embodiments, L is

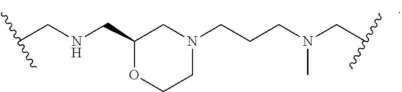 [SMILES: *CNC[C@H]1CN(CCCN(C)C*)CCO1]

In some embodiments, L is

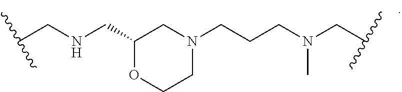 [SMILES: *CNC[C@@H]1CN(CCCN(C)C*)CCO1]

In some embodiments, L is

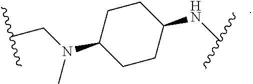 [SMILES: *CN(C)[C@H]1CC[C@@H](N*)CC1]

In some embodiments, L is

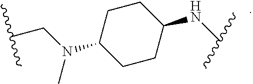 [SMILES: *CN(C)[C@H]1CC[C@H](N*)CC1]

In some embodiments, L is

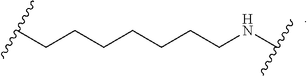 [SMILES: *CCCCCCCN*]

In some embodiments, L is

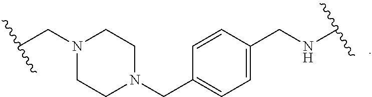 [SMILES: *CN1CCN(Cc2ccc(CN*)cc2)CC1]

some embodiments, L is

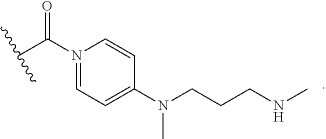

In some embodiments, L is

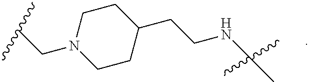 [SMILES: *CN1CCC(CCN*)CC1]

In some embodiments, L is

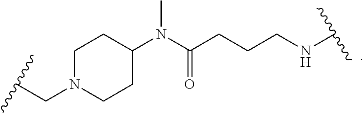 [SMILES: *CN1CCC(N(C)C(=O)CCCN*)CC1]

In some embodiments, L is

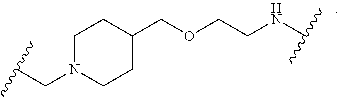 [SMILES: *CN1CCC(COCCN*)CC1]

In some embodiments, L is

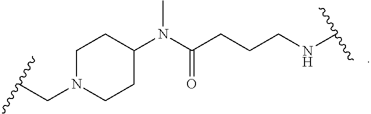 [SMILES: *CN1CCC(N(C)C(=O)CCCN*)CC1]

In some embodiments, L is

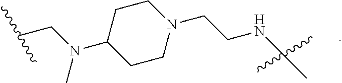 [SMILES: *CN(C)C1CCN(CCN*)CC1]

In some embodiments, L is

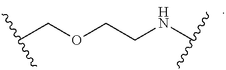 [SMILES: *COCCN*]

In some embodiments, L is

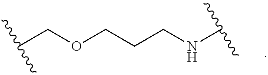 [SMILES: *COCCCN*]

In some embodiments, L is

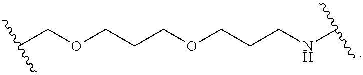 [SMILES: *COCCCOCCCN*]

In some embodiments, L is

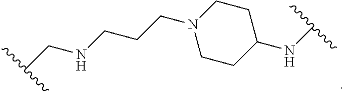 [SMILES: *CNCCCN1CCC(N*)CC1]

In some embodiments, L is a covalent bond. In some embodiments, L is

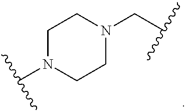 [SMILES: *CN1CCN(*)CC1]

In some embodiments, L is

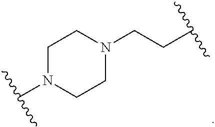 [SMILES: *CCN1CCN(*)CC1]

In some embodiments, L is

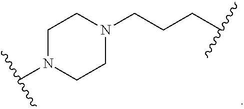 [SMILES: *CCCN1CCN(*)CC1]

In some embodiments, L is

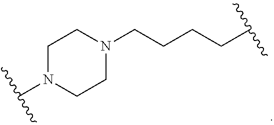 [SMILES: *CCCCN1CCN(*)CC1]

In some embodiments, L is

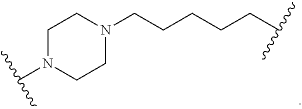 [SMILES: *CCCCCN1CCN(*)CC1]

In some embodiments, L is

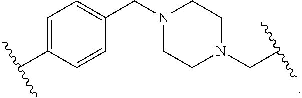 [SMILES: *CN1CCN(Cc2ccc(*)cc2)CC1]

In some embodiments, L is

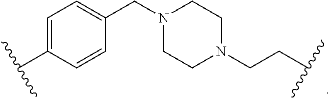 [SMILES: *CCN1CCN(Cc2ccc(*)cc2)CC1]

In some embodiments, L is

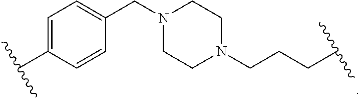 [SMILES: *CCCN1CCN(Cc2ccc(*)cc2)CC1]

In some embodiments, L is

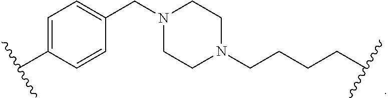 [SMILES: *CCCCN1CCN(Cc2ccc(*)cc2)CC1]

In some embodiments, L is

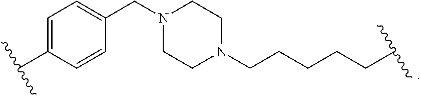 [SMILES: *CCCCCN1CCN(Cc2ccc(*)cc2)CC1]

In some embodiments, L is

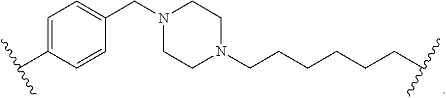 [SMILES: *CCCCCCN1CCN(Cc2ccc(*)cc2)CC1]

In some embodiments, L is

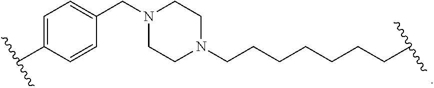 [SMILES: *CCCCCCCN1CCN(Cc2ccc(*)cc2)CC1]

In some embodiments, L is

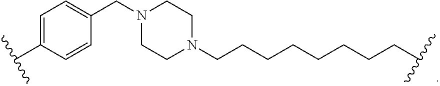 [SMILES: *CCCCCCCCN1CCN(Cc2ccc(*)cc2)CC1]

In some embodiments, L is

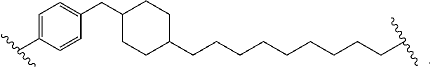 [SMILES: *CCCCCCCCCC1CCC(Cc2ccc(*)cc2)CC1]

In some embodiments, L is

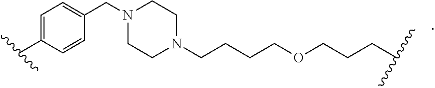 [SMILES: *CCCOCCCCN1CCN(Cc2ccc(*)cc2)CC1]

some embodiments, L is

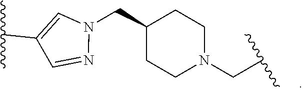

In some embodiments, L is

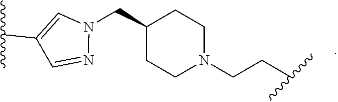

In some embodiments, L is

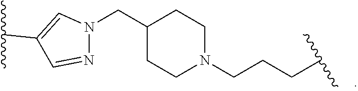 [SMILES: *CCCN1CCC(Cn2cc(*)cn2)CC1]

In some embodiments, L is

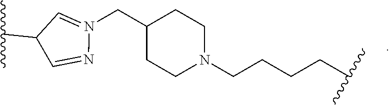

In some embodiments, L is

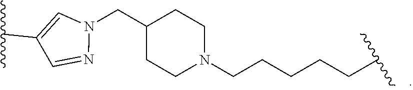 [SMILES: *CCCCCN1CCC(Cn2cc(*)cn2)CC1]

In some embodiments, L is

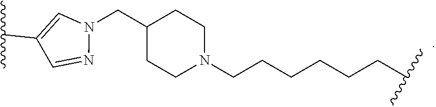 [SMILES: *CCCCCCN1CCC(Cn2cc(*)cn2)CC1]

In some embodiments, L is

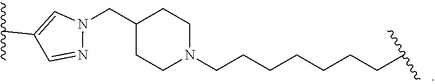 [SMILES: *CCCCCCCN1CCC(Cn2cc(*)cn2)CC1]

In some embodiments, L is

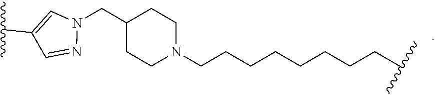 [SMILES: *CCCCCCCCN1CCC(Cn2cc(*)cn2)CC1]

In some embodiments, L is

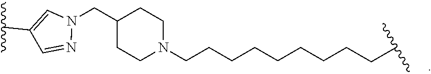 [SMILES: *CCCCCCCCCN1CCC(Cn2cc(*)cn2)CC1]

In some embodiments, L is

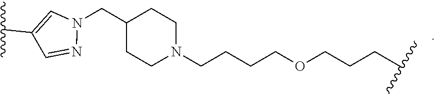 [SMILES: *CCCOCCCCN1CCC(Cn2cc(*)cn2)CC1]

In some embodiments, L is

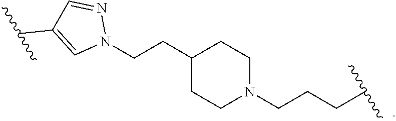 [SMILES: *CCCN1CCC(CCn2cc(*)cn2)CC1]

In some embodiments, L is

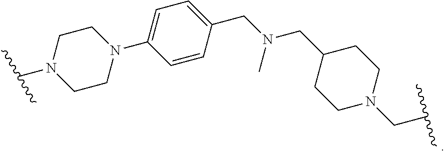 [SMILES: *CN1CCC(CN(C)Cc2ccc(N3CCN(*)CC3)cc2)CC1]

In some embodiments, L is

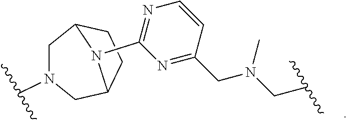 [SMILES: *CN(C)Cc1ccnc(N2C3CCC2CN(*)C3)n1]

In some embodiments, L is

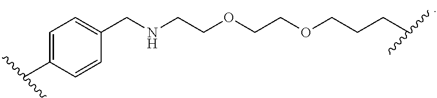 [SMILES: *CCCOCCOCCNCc1ccc(*)cc1]

In some embodiments, L is

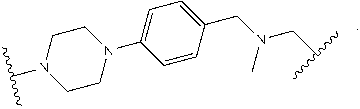 [SMILES: *CN(C)Cc1ccc(N2CCN(*)CC2)cc1]

In some embodiments, L is

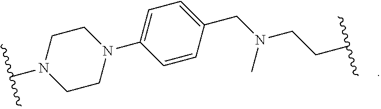 [SMILES: *CCN(C)Cc1ccc(N2CCN(*)CC2)cc1]

In some embodiments, L is

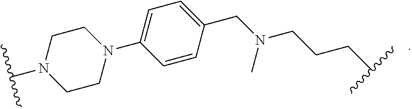 [SMILES: *CCCN(C)Cc1ccc(N2CCN(*)CC2)cc1]

In some embodiments, L is

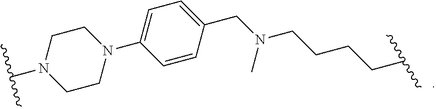 [SMILES: *CCCCN(C)Cc1ccc(N2CCN(*)CC2)cc1]

In some embodiments, L is

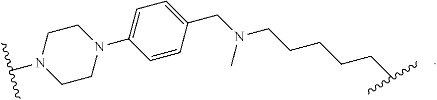 [SMILES: *CCCCCN(C)Cc1ccc(N2CCN(*)CC2)cc1]

In some embodiments, L is

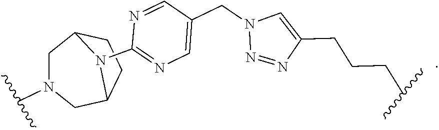 [SMILES: *CCCc1cn(Cc2cnc(N3C4CCC3CN(*)C4)nc2)nn1]

In some embodiments, L is

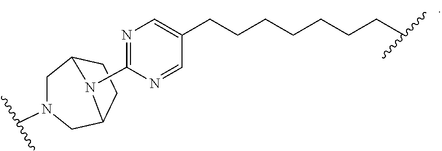 [SMILES: *CCCCCCCc1cnc(N2C3CCC2CN(*)C3)nc1]

In some embodiments, L is

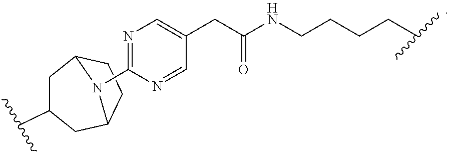 [SMILES: *CCCCNC(=O)Cc1cnc(N2C3CCC2CC(*)C3)nc1]

In some embodiments, L is

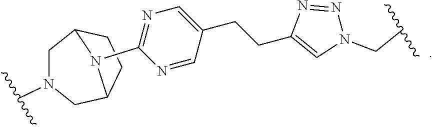 [SMILES: *Cn1cc(CCc2cnc(N3C4CCC3CN(*)C4)nc2)nn1]

In some embodiments, L is

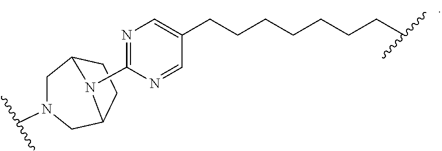 [SMILES: *CCCCCCCc1cnc(N2C3CCC2CN(*)C3)nc1]

In some embodiments, L is

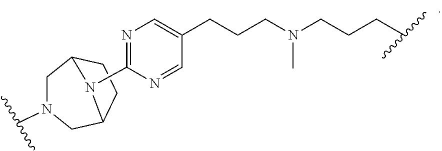 [SMILES: *CCCN(C)CCCc1cnc(N2C3CCC2CN(*)C3)nc1]

In some embodiments, L is

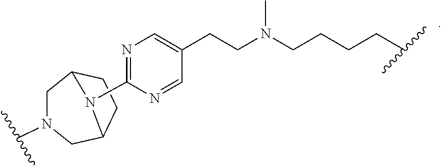 [SMILES: *CCCCN(C)CCc1cnc(N2C3CCC2CN(*)C3)nc1]

In some embodiments, L is

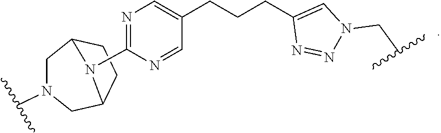 [SMILES: *Cn1cc(CCCc2cnc(N3C4CCC3CN(*)C4)nc2)nn1]

In some embodiments, L is

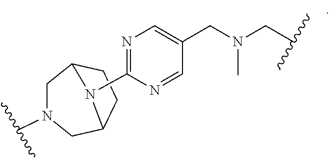 [SMILES: *CN(C)Cc1cnc(N2C3CCC2CN(*)C3)nc1]

In some embodiments, L is In

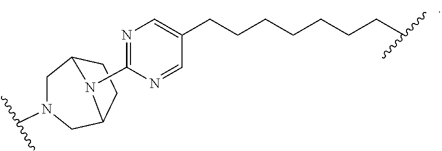 [SMILES: *CCCCCCCc1cnc(N2C3CCC2CN(*)C3)nc1]

some embodiments, L is

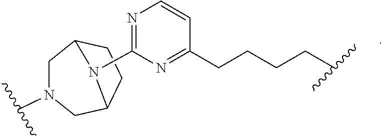 [SMILES: *CCCCc1ccnc(N2C3CCC2CN(*)C3)n1]

In some embodiments, L is

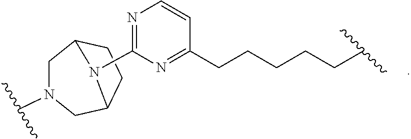 [SMILES: *CCCCCc1ccnc(N2C3CCC2CN(*)C3)n1]

In some embodiments, L is

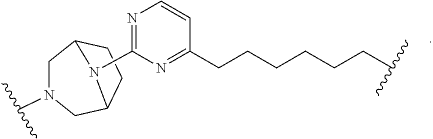 [SMILES: *CCCCCCc1ccnc(N2C3CCC2CN(*)C3)n1]

In some embodiments, L is

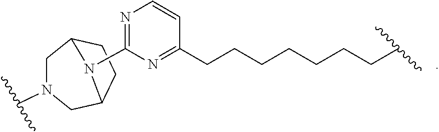 [SMILES: *CCCCCCCc1ccnc(N2C3CCC2CN(*)C3)n1]

In some embodiments, L is

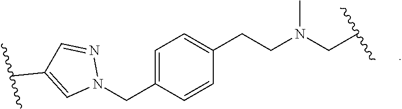 [SMILES: *CN(C)CCc1ccc(Cn2cc(*)cn2)cc1]

In some embodiments, L is

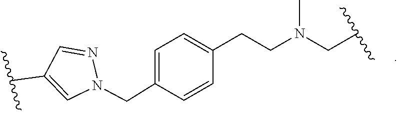 [SMILES: *CN(C)CCc1ccc(Cn2cc(*)cn2)cc1]

In some embodiments, L is

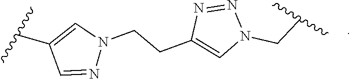 [SMILES: *Cn1cc(CCn2cc(*)cn2)nn1]

In some embodiments, L is

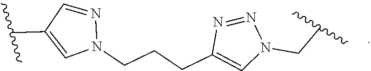 [SMILES: *Cn1cc(CCCn2cc(*)cn2)nn1]

In some embodiments, L is

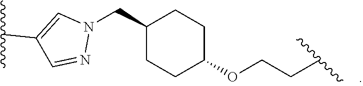 [SMILES: *CCO[C@H]1CC[C@H](Cn2cc(*)cn2)CC1]

In some embodiments, L is

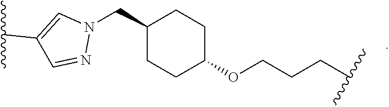 [SMILES: *CCCO[C@H]1CC[C@H](Cn2cc(*)cn2)CC1]

In some embodiments, L is

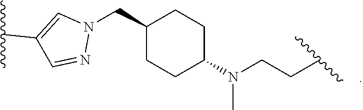 [SMILES: *CCN(C)[C@H]1CC[C@H](Cn2cc(*)cn2)CC1]

In some embodiments, L is

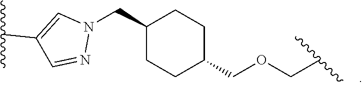 [SMILES: *COC[C@H]1CC[C@H](Cn2cc(*)cn2)CC1]

In some embodiments, L is

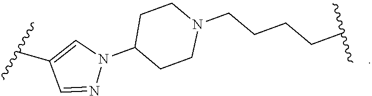 [SMILES: *CCCCN1CCC(n2cc(*)cn2)CC1]

In some embodiments, L is

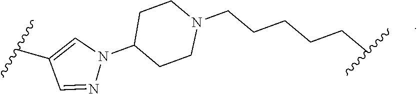 [SMILES: *CCCCCN1CCC(n2cc(*)cn2)CC1]

In some embodiments, L is

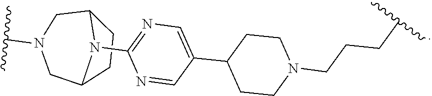 [SMILES: *CCCN1CCC(c2cnc(N3C4CCC3CN(*)C4)nc2)CC1]

In some embodiments, L is

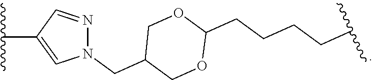 [SMILES: *CCCCC1OCC(Cn2cc(*)cn2)CO1]

In some embodiments, L is

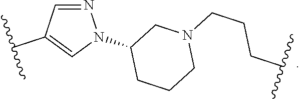 [SMILES: *CCCN1CCC[C@H](n2cc(*)cn2)C1]

In some embodiments, L is

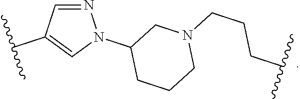 [SMILES: *CCCN1CCCC(n2cc(*)cn2)C1]

In some embodiments, L is

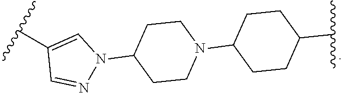 [SMILES: *c1cnn(C2CCN(C3CCC(*)CC3)CC2)c1]

In some embodiments, is

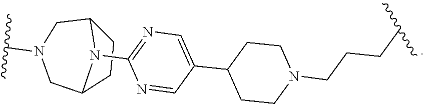 [SMILES: *CCCN1CCC(c2cnc(N3C4CCC3CN(*)C4)nc2)CC1]

In some embodiments, L is

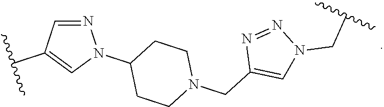 [SMILES: *Cn1cc(CN2CCC(n3cc(*)cn3)CC2)nn1]

In some embodiments, L is

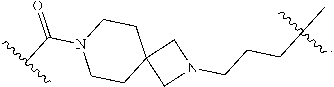 [SMILES: *CCCN1CC2(CCN(C(*)=O)CC2)C1]

In some embodiments, L is

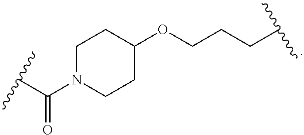 [SMILES: *CCCOC1CCN(C(*)=O)CC1]

In some embodiments, L is

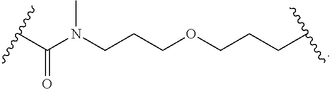 [SMILES: *CCCOCCCN(C)C(*)=O]

In some embodiments, L is

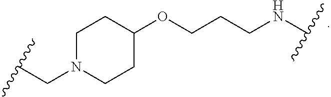 [SMILES: *CN1CCC(OCCCN*)CC1]

In some embodiments, L is

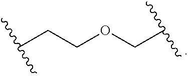 [SMILES: *CCOC*]

In some embodiments, L is

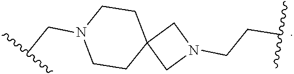 [SMILES: *CCN1CC2(CCN(C*)CC2)C1]

In some embodiments, L is

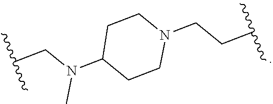 [SMILES: *CCN1CCC(N(C)C*)CC1]

In some embodiments, L is

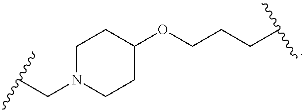 [SMILES: *CCCOC1CCN(C*)CC1]

In some embodiments, L is

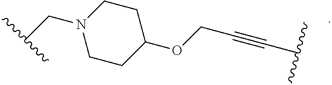 [SMILES: *C#CCOC1CCN(C*)CC1]

In some embodiments, L is

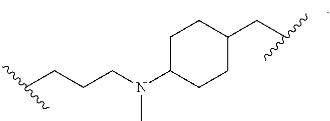 [SMILES: *CCCN(C)C1CCC(C*)CC1]

In some embodiments, L is

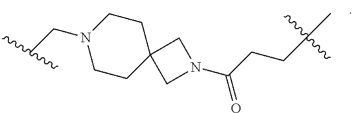 [SMILES: *CCC(=O)N1CC2(CCN(C*)CC2)C1]

In some embodiments, L is

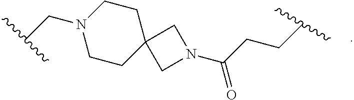 [SMILES: *CCC(=O)N1CC2(CCN(C*)CC2)C1]

In some embodiments, L is

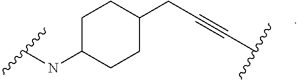 [SMILES: *C#CCC1CCC(N*)CC1]

n some embodiments, L is

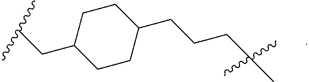 [SMILES: *CCCC1CCC(C*)CC1]

In some embodiments, L is

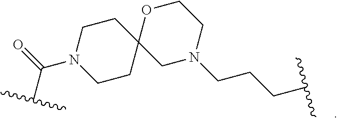 [SMILES: *CCCN1CCOC2(CCN(C(*)=O)CC2)C1]

In some embodiments, L is

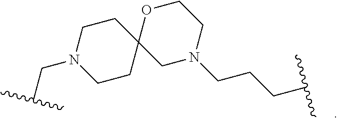 [SMILES: *CCCN1CCOC2(CCN(C*)CC2)C1]

In some embodiments, L is

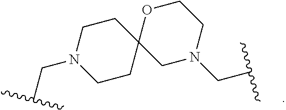 [SMILES: *CN1CCC2(CC1)CN(C*)CCO2]

In some embodiments, L is

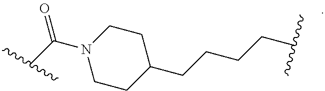 [SMILES: *CCCCC1CCN(C(*)=O)CC1]

In some embodiments, L is

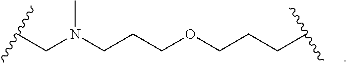 [SMILES: *CCCOCCCN(C)C*]

In some embodiments, L is

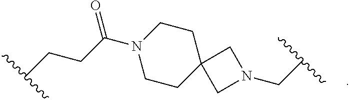 [SMILES: *CCC(=O)N1CCC2(CC1)CN(C*)C2]

In some embodiments, L is

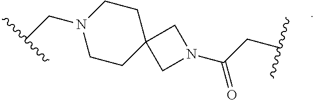 [SMILES: *CC(=O)N1CC2(CCN(C*)CC2)C1]

In some embodiments, L is

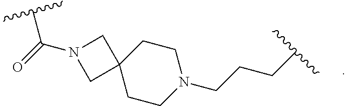 [SMILES: *CCCN1CCC2(CC1)CN(C(*)=O)C2]

In some embodiments, L is

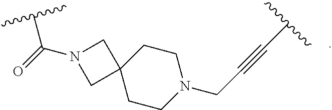 [SMILES: *C#CCN1CCC2(CC1)CN(C(*)=O)C2]

In some embodiments, L is

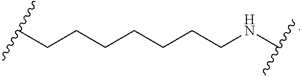 [SMILES: *CCCCCCCN*]

In some embodiments, L is

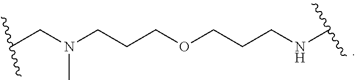 [SMILES: *CN(C)CCCOCCCN*]

In some embodiments, L is

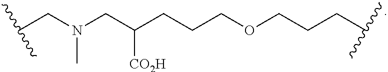 [SMILES: *CCCOCCCC(CN(C)C*)C(=O)O]

In some embodiments, L is

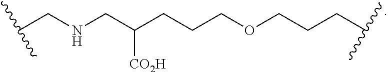 [SMILES: *CCCOCCCC(CNC*)C(=O)O]

In some embodiments, L is

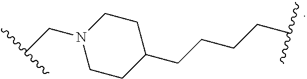 [SMILES: *CCCCC1CCN(C*)CC1]

In some embodiments, L is

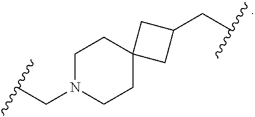 [SMILES: *CC1CC2(CCN(C*)CC2)C1]

In some embodiments, L is

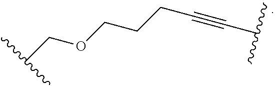 [SMILES: *C#CCCCOC*]

In some embodiments, L is

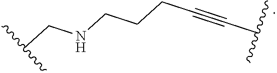 [SMILES: *C#CCCCNC*]

In some embodiments, L is

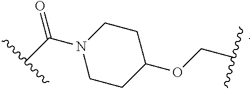 [SMILES: *COC1CCN(C(*)=O)CC1]

In some embodiments, L is

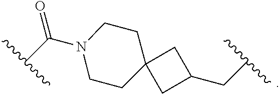 [SMILES: *CC1CC2(CCN(C(*)=O)CC2)C1]

In some embodiments, L is

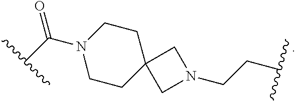 [SMILES: *CCN1CC2(CCN(C(*)=O)CC2)C1]

In some embodiments, L is

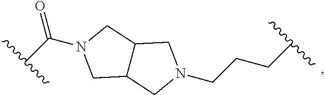 [SMILES: *CCCN1CC2CN(C(*)=O)CC2C1]

In some embodiments, L is

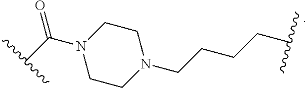 [SMILES: *CCCCN1CCN(C(*)=O)CC1]

In some embodiments, L is

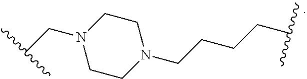 [SMILES: *CCCCN1CCN(C*)CC1]

In some embodiments, L is

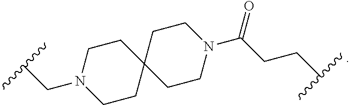 [SMILES: *CCC(=O)N1CCC2(CCN(C*)CC2)CC1]

In some embodiments, L is

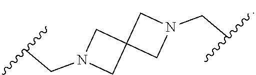 [SMILES: *CN1CC2(C1)CN(C*)C2]

In some embodiments, L is

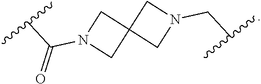 [SMILES: *CN1CC2(C1)CN(C(*)=O)C2]

In some embodiments, L is

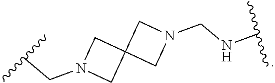 [SMILES: *CN1CC2(C1)CN(CN*)C2]

In some embodiments, L is

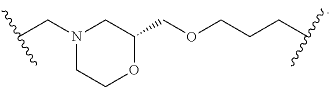 [SMILES: *CCCOC[C@H]1CN(C*)CCO1]

In some embodiments, L is

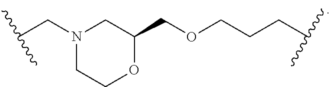 [SMILES: *CCCOC[C@@H]1CN(C*)CCO1]

In some embodiments, L is

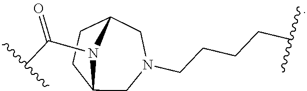 [SMILES: *CCCCN1C[C@H]2CC[C@@H](C1)N2C(*)=O]

In some embodiments, L is

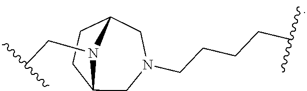 [SMILES: *CCCCN1C[C@H]2CC[C@@H](C1)N2C*]

In some embodiments, L is

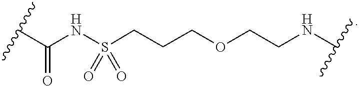 [SMILES: *NCCOCCCS(=O)(=O)NC(*)=O]

In some embodiments, L is

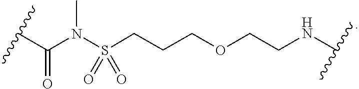 [SMILES: *NCCOCCCS(=O)(=O)N(C)C(*)=O]

In some embodiments, L is

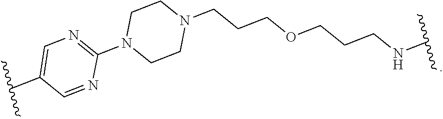 [SMILES: *NCCCOCCCN1CCN(c2ncc(*)cn2)CC1]

In some embodiments, L is

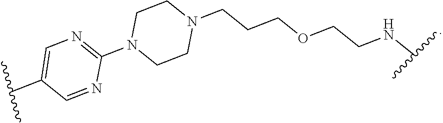 [SMILES: *NCCOCCCN1CCN(c2ncc(*)cn2)CC1]

In some embodiments, L is

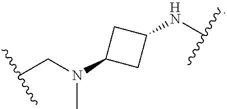 [SMILES: *CN(C)[C@H]1C[C@H](N*)C1]

In some embodiments, L is

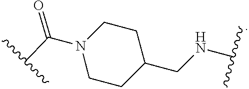 [SMILES: *NCC1CCN(C(*)=O)CC1]

In some embodiments, L is

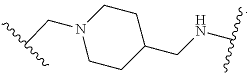 [SMILES: *CN1CCC(CN*)CC1]

In some embodiments, L is

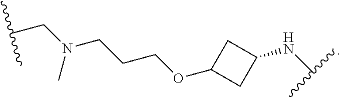

In some embodiments, L is

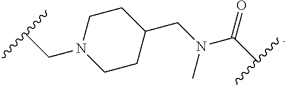 [SMILES: *CN1CCC(CN(C)C(*)=O)CC1]

In some embodiments, L is

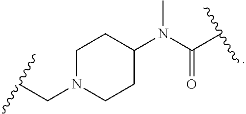 [SMILES: *CN1CCC(N(C)C(*)=O)CC1 ; "."]

In some embodiments, L is

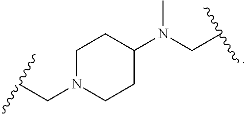 [SMILES: *CN1CCC(N(C)C*)CC1 ; "."]

In some embodiments, L is

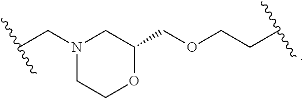 [SMILES: *CCOC[C@H]1CN(C*)CCO1 ; "."]

In some embodiments, L is

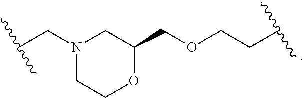 [SMILES: *CCOC[C@@H]1CN(C*)CCO1 ; "."]

In some embodiments, L is

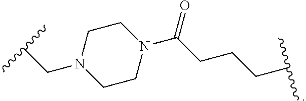 [SMILES: *CCCC(=O)N1CCN(C*)CC1 ; "."]

In some embodiments, L is

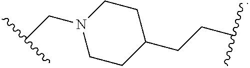 [SMILES: *CCC1CCN(C*)CC1 ; "."]

In some embodiments, L is

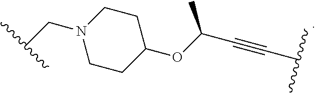 [SMILES: *C#C[C@H](C)OC1CCN(C*)CC1 ; "."]

In some embodiments, L is

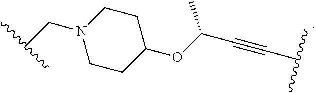 [SMILES: *C#C[C@@H](C)OC1CCN(C*)CC1 ; "."]

In some embodiments, L is

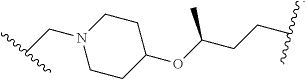 [SMILES: "." ; *CC[C@H](C)OC1CCN(C*)CC1]

In some embodiments, L is

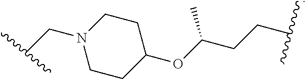 [SMILES: "." ; *CC[C@@H](C)OC1CCN(C*)CC1]

In some embodiments, L is

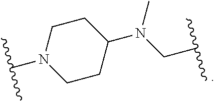 [SMILES: *CN(C)C1CCN(*)CC1 ; "."]

In some embodiments, L is

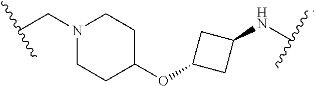 [SMILES: "." ; *CN1CCC(O[C@H]2C[C@H](N*)C2)CC1]

In some embodiments, L is

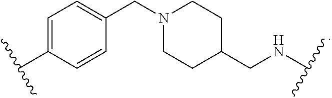 [SMILES: *NCC1CCN(Cc2ccc(*)cc2)CC1 ; "."]

In some embodiments, L is

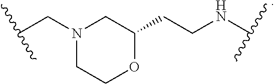 [SMILES: "." ; *CN1CCO[C@@H](CCN*)C1]

In some embodiments, L is

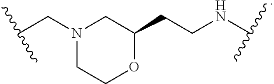 [SMILES: "." ; *CN1CCO[C@H](CCN*)C1]

In some embodiments, L is

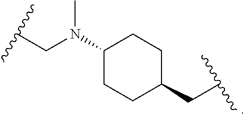 [SMILES: *CN(C)[C@H]1CC[C@H](C*)CC1 ; "."]

In some embodiments, L is

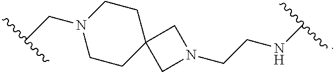 [SMILES: *CN1CCC2(CC1)CN(CCN*)C2 ; "."]

In some embodiments, L is

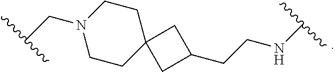 [SMILES: *CN1CCC2(CC1)CC(CCN*)C2 ; "."]

In some embodiments, L is

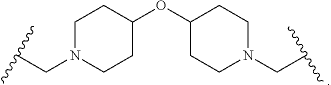 [SMILES: *CN1CCC(OC2CCN(C*)CC2)CC1 ; "."]

In some embodiments, L is

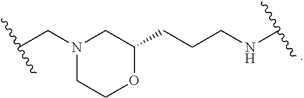 [SMILES: *CN1CCO[C@@H](CCCN*)C1 ; "."]

In some embodiments, L is

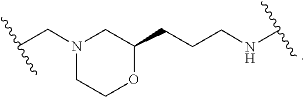 [SMILES: *CN1CCO[C@H](CCCN*)C1 ; "."]

In some embodiments, L is

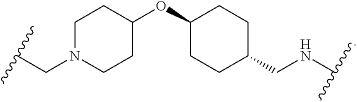 [SMILES: *CN1CCC(O[C@H]2CC[C@H](CN*)CC2)CC1]

In some embodiments, L is

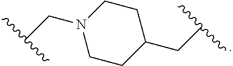 [SMILES: *CC1CCN(C*)CC1 ; "."]

In some embodiments, L is

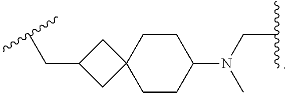 [SMILES: *CC1CC2(CCC(N(C)C*)CC2)C1 ; "."]

In some embodiments, L is

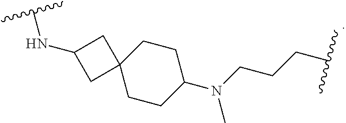 [SMILES: *CCCN(C)C1CCC2(CC1)CC(N*)C2 ; "."]

In some embodiments, L is

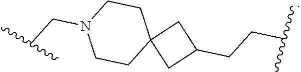 [SMILES: *CCC1CC2(CCN(C*)CC2)C1 ; "."]

In some embodiments, L is

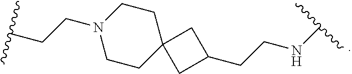 [SMILES: *CCN1CCC2(CC1)CC(CCN*)C2 ; "."]

In some embodiments, L is

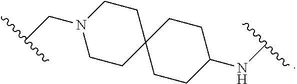 [SMILES: *CN1CCC2(CCC(N*)CC2)CC1 ; "."]

In some embodiments, L is

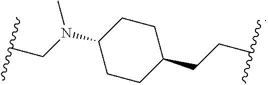 [SMILES: *CC[C@H]1CC[C@H](N(C)C*)CC1 ; "."]

In some embodiments, L is

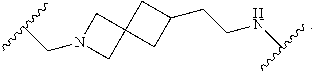 [SMILES: *CN1CC2(CC(CCN*)C2)C1]

In some embodiments, L is

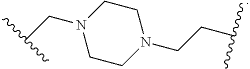 [SMILES: *CCN1CCN(C*)CC1 ; "."]

In some embodiments, L is

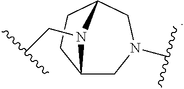 [SMILES: *CN1[C@@H]2CC[C@H]1CN(*)C2 ; "."]

In some embodiments, L is

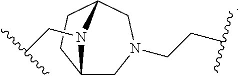 [SMILES: *CCN1C[C@H]2CC[C@@H](C1)N2C* ; "."]

In some embodiments, L is

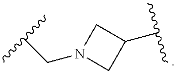 [SMILES: *CN1CC(*)C1 ; "."]

In some embodiments, L is

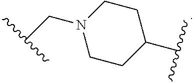 [SMILES: *CN1CCC(*)CC1 ; "."]

In some embodiments, L is

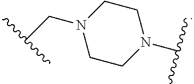 [SMILES: *CN1CCN(*)CC1 ; "."]

In some embodiments, L is

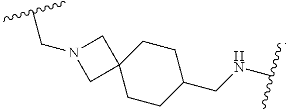 [SMILES: *CN1CC2(CCC(CN*)CC2)C1 ; "."]

In some embodiments, L is

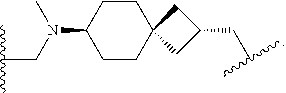

In some embodiments, L is

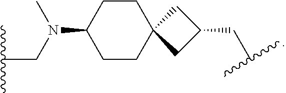

In some embodiments, L is

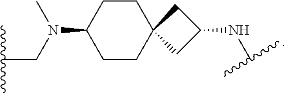

In some embodiments, L is

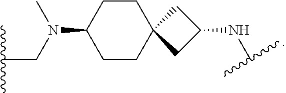

In some embodiments, L is

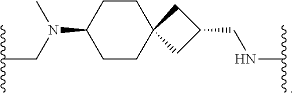

In some embodiments, L is

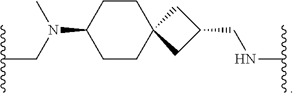

In some embodiments, L is

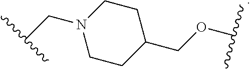 [SMILES: *CN1CCC(CO*)CC1 ; "."]

In some embodiments, L is

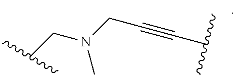 [SMILES: *C#CCN(C)C*]

In some embodiments, L is

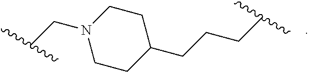 [SMILES: *CCCC1CCN(C*)CC1]

In some embodiments, L is

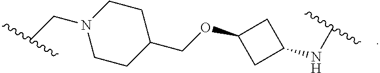 [SMILES: *CN1CCC(CO[C@H]2C[C@H](N*)C2)CC1]

In some embodiments, L is

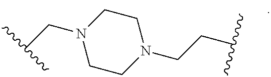 [SMILES: *CCN1CCN(C*)CC1]

In some embodiments, L is

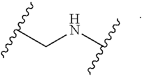 [SMILES: *CN*]

In some embodiments, L is

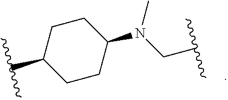 [SMILES: *CN(C)[C@H]1CC[C@@H](*)CC1]

In some embodiments, L is

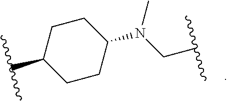 [SMILES: *CN(C)[C@H]1CC[C@H](*)CC1]

In some embodiments, L is

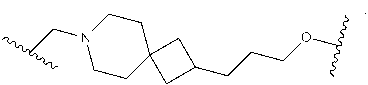 [SMILES: *CN1CCC2(CC1)CC(CCCO*)C2]

In some embodiments, L is

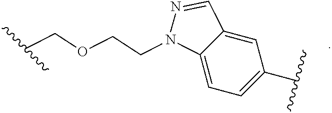 [SMILES: *COCCn1ncc2cc(*)ccc21]

In some embodiments, L is

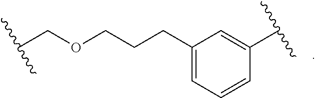 [SMILES: *COCCCc1cccc(*)c1]

In some embodiments, L is

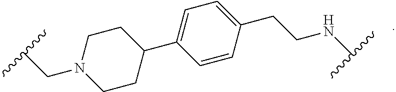 [SMILES: *CN1CCC(c2ccc(CCN*)cc2)CC1]

In some embodiments, L is

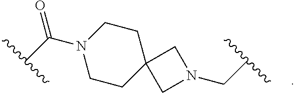 [SMILES: *CN1CC2(CCN(C(*)=O)CC2)C1]

In some embodiments, L is

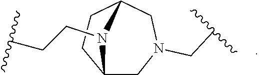 [SMILES: *CCN1[C@@H]2CC[C@H]1CN(C*)C2]

In some embodiments, L is

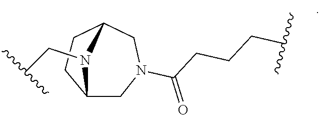 [SMILES: *CCCC(=O)N1C[C@H]2CC[C@@H](C1)N2C*]

In some embodiments, L is

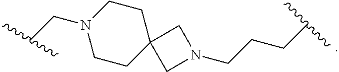 [SMILES: *CCCN1CC2(CCN(C*)CC2)C1]

In some embodiments, L is

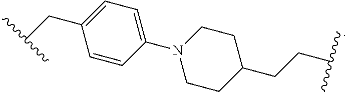 [SMILES: *CCC1CCN(c2ccc(C*)cc2)CC1]

In some embodiments, L is

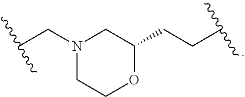 [SMILES: *CC[C@H]1CN(C*)CCO1]

In some embodiments, L is

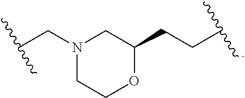 [SMILES: *CC[C@@H]1CN(C*)CCO1]

In some embodiments, L is

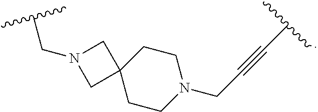 [SMILES: *C#CCN1CCC2(CC1)CN(C*)C2]

In some embodiments, L is

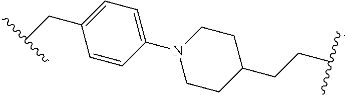 [SMILES: *CCC1CCN(c2ccc(C*)cc2)CC1]

In some embodiments, L is

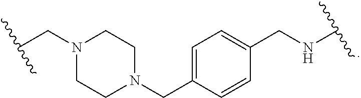 [SMILES: *CN1CCN(Cc2ccc(CN*)cc2)CC1]

In some embodiments, L is

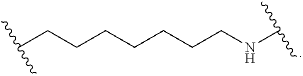 [SMILES: *CCCCCCCN*]

In some embodiments, L is

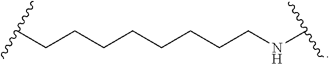 [SMILES: *CCCCCCCCN*]

In some embodiments, L is

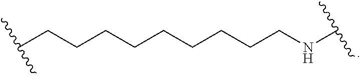 [SMILES: *CCCCCCCCCN*]

In some embodiments, L is

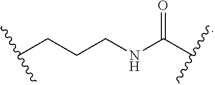 [SMILES: *CCCNC(*)=O]

In some embodiments, L is

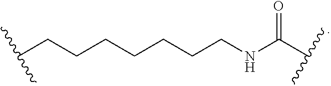 [SMILES: *CCCCCCCNC(*)=O]

In some embodiments, L is

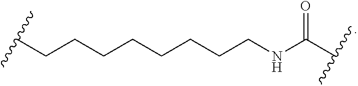 [SMILES: *CCCCCCCCNC(*)=O]

In some embodiments, L is

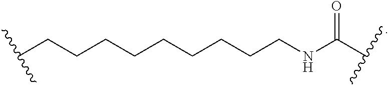 [SMILES: *CCCCCCCCCNC(*)=O]

In some embodiments, L is

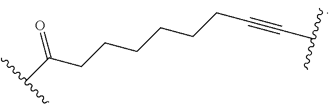 [SMILES: *C#CCCCCCCC(*)=O]

In some embodiments, L is

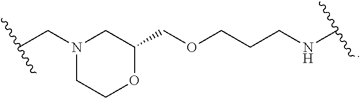 [SMILES: *CN1CCO[C@@H](COCCCN*)C1]

In some embodiments, L is

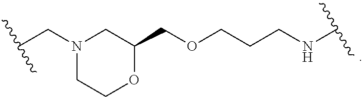 [SMILES: *CN1CCO[C@H](COCCCN*)C1]

In some embodiments, L is

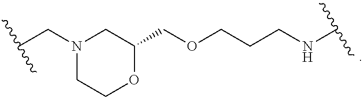 [SMILES: *CN1CCO[C@@H](COCCCN*)C1]

In some embodiments, L is

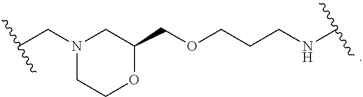 [SMILES: *CN1CCO[C@H](COCCCN*)C1]

In some embodiments, L is

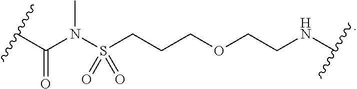 [SMILES: *NCCOCCCS(=O)(=O)N(C)C(*)=O]

In some embodiments, L is

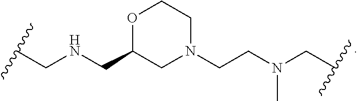 [SMILES: *CNC[C@@H]1CN(CCN(C)C*)CCO1]

In some embodiments, L is

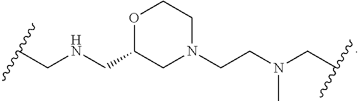 [SMILES: *CNC[C@H]1CN(CCN(C)C*)CCO1]

In some embodiments, L is

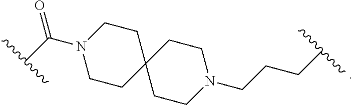 [SMILES: *CCCN1CCC2(CC1)CCN(C(*)=O)CC2]

In some embodiments, L is

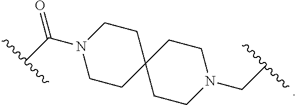 [SMILES: *CN1CCC2(CC1)CCN(C(*)=O)CC2]

In some embodiments, L is

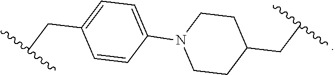 [SMILES: *Cc1ccc(N2CCC(C*)CC2)cc1]

In some embodiments, L is

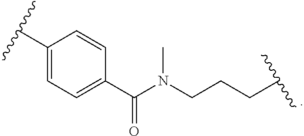 [SMILES: *CCCN(C)C(=O)c1ccc(*)cc1]

In some embodiments, L is

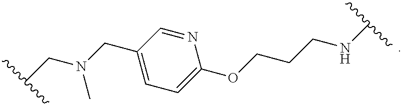 [SMILES: *CN(C)Cc1ccc(OCCCN*)nc1]

In some embodiments, L is

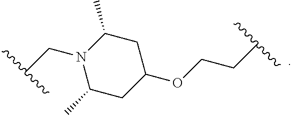 [SMILES: *CCOC1C[C@@H](C)N(C*)[C@@H](C)C1]

In some embodiments, L is

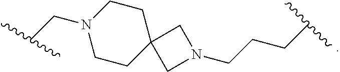 [SMILES: *CCCN1CC2(CCN(C*)CC2)C1]

In some embodiments, L is

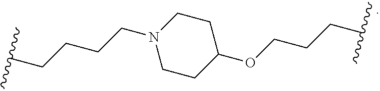 [SMILES: *CCCCN1CCC(OCCC*)CC1]

In some embodiments, L is

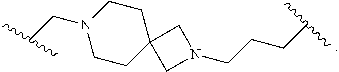 [SMILES: *CCCN1CC2(CCN(C*)CC2)C1]

In some embodiments, L is

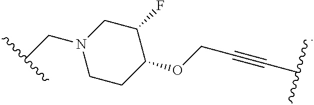 [SMILES: *C#CCO[C@@H]1CCN(C*)C[C@@H]1F]

In some embodiments, L is

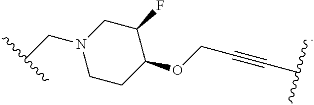 [SMILES: *C#CCO[C@H]1CCN(C*)C[C@H]1F]

In some embodiments, L is

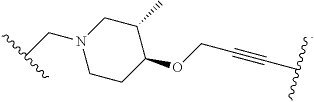 [SMILES: *C#CCO[C@H]1CCN(C*)C[C@@H]1C]

In some embodiments, L is

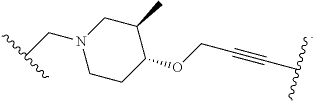 [SMILES: *C#CCO[C@@H]1CCN(C*)C[C@H]1C]

In some embodiments, L is

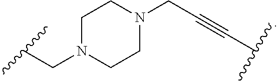 [SMILES: *C#CCN1CCN(C*)CC1]

In some embodiments, L is selected from those depicted in Table B, below.

In some embodiments, L is selected from those depicted in Table 1, below.

Without limitation, the point of attachment of L to TBM and KBM can be, for example when L is

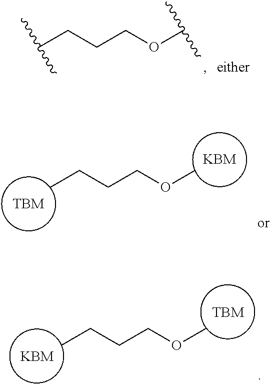

### Target Binding Moiety (TBM)

As defined above and described herein, TBM is a target binding moiety.

In some embodiments, TBM is a target binding moiety.

As described herein, wherein a formula is depicted using square brackets, e.g.,

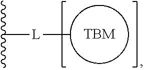

L is attached to a modifiable carbon, oxygen, or nitrogen atom within TBM including substitution or replacement of a defined group in TBM.

In preferred aspects of the invention, the TBM group is a group, which binds to target proteins. Targets of the TBM group are numerous in kind and are selected from proteins that are expressed in a cell such that at least a portion of the sequences is found in the cell and may bind to a TBM group. The term “protein” includes oligopeptides and polypeptide sequences of sufficient length that they can bind to a TBM group according to the present invention. Any protein in a eukaryotic system, as described herein, are targets for ubiquitination mediated by the compounds according to the present invention.

TBM groups according to the present invention include, for example, include any moiety which binds to a protein specifically (binds to a target protein) and includes the following non-limiting examples of small molecule target protein moieties: Hsp90 inhibitors, kinase inhibitors, HDM2 & MDM2 inhibitors, compounds targeting Human BET Bromodomain-containing proteins, HDAC inhibitors, human lysine methyltransferase inhibitors, angiogenesis inhibitors, nuclear hormone receptor compounds, immunosuppressive compounds, and compounds targeting the aryl hydrocarbon receptor (AHR), among numerous others. The compositions described below exemplify some of the members of these nine types of small molecule target protein binding moieties. Such small molecule target protein binding moieties also include pharmaceutically acceptable salts, enantiomers, solvates and polymorphs of these compositions, as well as other small molecules that may target a protein of interest. These binding moieties are linked to the ubiquitin ligase binding moiety preferably through a linker in order to present a target protein (to which the protein target moiety is bound) in proximity to the ubiquitin ligase for ubiquitination and degradation.

Any protein, which can bind to a target binding moiety or TBM group and acted on or degraded by an ubiquitin ligase is a target protein according to the present invention. In general, target proteins may include, for example, structural proteins, receptors, enzymes, cell surface proteins, proteins pertinent to the integrated function of a cell, including proteins involved in catalytic activity, aromatase activity, motor activity, helicase activity, metabolic processes (anabolism and catabolismantioxidant activity, proteolysis, biosynthesis, proteins with kinase activity, oxidoreductase activity, transferase activity, hydrolase activity, lyase activity, isomerase activity, ligase activity, enzyme regulator activity, signal transducer activity, structural molecule activity, binding activity (protein, lipid carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport (including protein transporter activity, nuclear transport, ion transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity, translation regulator activity. Proteins of interest can include proteins from eurkaryotes and prokaryotes including humans as targets for drug therapy, other animals, including domesticated animals, microbials for the determination of targets for antibiotics and other antimicrobials and plants, and even viruses, among numerous others.

In some embodiments, TBM (or target binding moiety) is a small molecule which is capable of binding to or binds to a target protein of interest. Some embodiments of the present application relates to TBMs which include but are not limited to Hsp90 inhibitors, kinase inhibitors, STAT3 inhibitors, compounds targeting Human BET Bromodomain-containing proteins, compounds targeting cytosolic signaling protein FKBP12, HDAC inhibitors, human lysine methyltransferase inhibitors, angiogenesis inhibitors, immunosuppressive compounds, and compounds targeting the aryl hydrocarbon receptor (AHR).

In some embodiments, TBM is STAT3 binding moiety of formula I-aa:

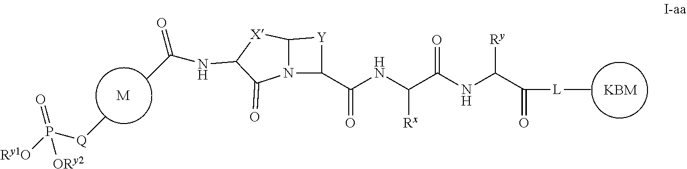

or a pharmaceutically acceptable salt thereof, wherein L and KBM are as defined above and described in embodiments herein, and wherein:
X′ is an optionally substituted —(CH₂)ₓ—, wherein 1-2 methylenes of X is optionally replaced with a bivalent group selected from —NR—, —N(COR)—, —N(CO₂R)—, —N(SO₂R)—, —N(CONR₂)—, and —N (SO₂NR₂)—, wherein:
   x is 1, 2, 3, 4, or 5;
each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-11 membered saturated or partially unsaturated monocyclic, bicyclic, bridged bicyclic, or spirocyclic carbocyclic or heterocyclic ring having 1-3 heteroatoms, in addition to the carbon or nitrogen from which the two R groups are attached, independently selected from nitrogen, oxygen, and sulfur;
Y is an optionally substituted —(CH₂)_{y}—, wherein:
   y is 1, 2, or 3;
R^{x} is hydrogen, R^{A}, —(CR₂)₁₋₃₀CONR₂, or —(CR₂)₁₋₃CONR₂;
each R^{A} is independently an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R^{y} is hydrogen, R^{A}, or

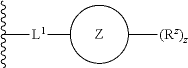

L′ is a covalent bond or a bivalent, saturated or partially unsaturated, straight or branched C₁₋₅ hydrocarbon chain, wherein 0-3 methylene units of L¹ are independently replaced by —O—, —NR—, —CRF—, —CF₂—, —C(O)—, —S—, —S(O)—, or —S(O)₂—;
Ring Z is a ring selected from phenyl, naphthyl, a 5-10 membered heteroaryl containing 1-4 heteroatoms independently selected from nitrogen, oxygen, or sulfur, a 5-11 membered saturated or partially unsaturated carbocyclyl or heterocyclyl with 1-4 heteroatoms independently selected from nitrogen, oxygen, or sulfur;
R^{z′} is hydrogen, R^{A}, halogen, —CN, —NO₂, —OR, —SR, —NR₂, —
   SiR₃, —S(O)₂R, —S(O)₂NR₂, —S(O)R, —C(O)R, —C(O) OR, —C(O)NR₂, —C(O)NROR, —CR₂NRC(O)R, —CR₂NRC(O)NR₂, —OC(O)R, —OC(O)NR₂, —OP(O)R², —OP(O)(OR)₂, —OP(O)(OR)NR₂, —OP(O)(NR₂)₂, —NRC(O) OR, —NRC(O)R, —NRC(O)NR₂, —NRS (O)₂R, —NP(O)R², —NRP(O)(OR)₂, —NRP(O)(OR)NR₂, —NRP(O)(NR₂)₂, or —NRS (O)₂R;
z is 0, 1, 2, 3, or 4;
Ring M is an optionally substituted bivalent ring selected from phenylenyl, naphthylenyl, a 5-10 membered heteroarylenyl containing 1-4 heteroatoms independently selected from nitrogen, oxygen, or sulfur, a 5-11 membered saturated or partially unsaturated carbocyclylenyl or heterocyclylenyl with 1-4 heteroatoms independently selected from nitrogen, oxygen, or sulfur;
Q is a bivalent moiety selected from —O—, —CR₂—, —CF₂—, —CFR—, —C(O)—, —OCR₂—, and —C(S)—; and
R^{y1} and R^{y2} are each independently hydrogen, R^{A}, —CH₂CO₂R, or —CH₂OCO₂R.

In some embodiments, TBM is STAT3 binding moiety of formula I-bb:

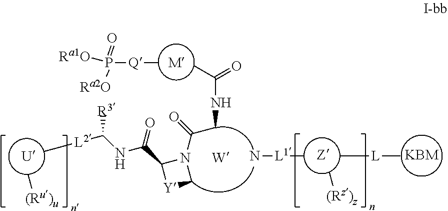

or a pharmaceutically acceptable salt thereof, wherein L and KBM are as defined above and described in embodiments herein, and wherein:
L^{1′} is a covalent bond or a bivalent, saturated or partially unsaturated, straight or branched C₁₋₅ hydrocarbon chain, wherein 0-3 methylene units of L^{1′} are independently replaced by —O—, —NR—, —CRF—, —CF₂—, —C(O)—, —S—, —S(O)—, or —S(O)₂—;
L^{2′} is a covalent bond or a bivalent, saturated or partially unsaturated, straight or branched C₁₋₅ hydrocarbon chain, wherein 0-3 methylene units of L^{2′} are independently replaced by —O—, —NR—, —CRF—, —CF₂—, —C(O)—, —S—, —S(O)—, or —S(O)₂—;
each R is independently hydrogen, or an optionally substituted group selected from C₁₋₆ aliphatic, phenyl, a 3-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-11 membered saturated or partially unsaturated monocyclic, bicyclic, bridged bicyclic, or spirocyclic carbocyclic or heterocyclic ring having 1-3 heteroatoms, in addition to the carbon or nitrogen from which the two R groups are attached, independently selected from nitrogen, oxygen, and sulfur;
R^{3′} is hydrogen or R^{A},
each R^{A} is independently an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring M′ is an optionally substituted bivalent ring selected from phenylenyl, naphthylenyl, a 5-10 membered heteroarylenyl containing 1-4 heteroatoms independently selected from nitrogen, oxygen, or sulfur, a 5-11 membered saturated or partially unsaturated carbocyclylenyl or heterocyclylenyl with 1-4 heteroatoms independently selected from nitrogen, oxygen, or sulfur;
Q′ is a bivalent moiety selected from —O—, —CR₂—, —CF₂—, —CFR—, —C(O)—, —OCR₂—, and —C(S)—;
R^{a1} and R^{a2} are each independently hydrogen, R^{A}, —CH₂CO₂R, or —CH₂OCO₂R;
Y′ is an optionally substituted —(CH₂)_{y}—, wherein:
   y is 1, 2, or 3;
Ring W′ is an optionally substituted ring selected from a 5-9 membered saturated or partially unsaturated heterocyclyl;
Ring U′ is a ring selected from phenyl, a 5-6 membered heteroaryl containing 1-4 heteroatoms independently selected from nitrogen, oxygen, or sulfur, and a 5-7 membered saturated or partially unsaturated carbocyclyl or heterocyclyl with 1-3 heteroatoms independently selected from nitrogen, oxygen, or sulfur;
R^{u′} is hydrogen, R^{A}, halogen, —CN, —NO₂, —OR, —SR, —NR₂, —
   SiR₃, —S(O)₂R, —S(O)₂NR₂, —S(O)R, —C(O)R, —C(O) OR, —C(O)NR₂, —C(O)NROR, —CR₂NRC(O)R, —CR₂NRC(O)NR₂, —OC(O)R, —OC(O)NR₂, —OP(O)R², —OP(O)(OR)₂, —OP(O)(OR)NR₂, —OP(O)(NR₂)₂, —NRC(O) OR, —NRC(O)R, —NRC(O)NR₂, —NRS (O)₂R, —NP(O)R², —NRP(O)(OR)₂, —NRP(O)(OR)NR₂, —NRP(O)(NR₂)₂, or —NRS (O)₂R;
u is 0, 1, 2, 3, or 4;
Ring Z′ is a bivalent ring selected from phenylenyl, a 4-7 membered saturated or partially unsaturated carbocyclylenyl or heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur;
R^{z′} is hydrogen, R^{A}, halogen, —CN, —NO₂, —OR, —SR, —NR₂, —
   SiR₃, —S(O)₂R, —S(O)₂NR₂, —S(O)R, —C(O)R, —C(O) OR, —C(O)NR₂, —C(O)NROR, —CR₂NRC(O)R, —CR₂NRC(O)NR₂, —OC(O)R, —OC(O)NR₂, —OP(O)R², —OP(O)(OR)₂, —OP(O)(OR)NR₂, —OP(O)(NR₂)₂, —NRC(O) OR, —NRC(O)R, —NRC(O)NR₂, —NRS (O)₂R, —NP(O)R², —NRP(O)(OR)₂, —NRP(O)(OR)NR₂, —NRP(O)(NR₂)₂, or —NRS (O)₂R;
z is 0, 1, 2, 3, or 4;
n is 0 or 1; and
n′ is 1 or 2.

As defined above and described herein, L^{1′} is a covalent bond or a bivalent, saturated or partially unsaturated, straight or branched C₁₋₅ hydrocarbon chain, wherein 0-3 methylene units of L^{1′} are independently replaced by —O—, —NR—, —CRF—, —CF₂—, —C(O)—, —S—, —S(O)—, or —S(O)₂—.

In some embodiments, L^{1′} is covalent bond. In some embodiments, L^{1′} is a bivalent, saturated or partially unsaturated, straight or branched C₁₋₅ hydrocarbon chain, wherein 0-3 methylene units of L^{1′} are independently replaced by —O—, —NR—, —CRF—, —CF₂—, —C(O)—, —S—, —S(O)—, or —S(O)₂—.

In some embodiments, L^{1′} is selected from those depicted in Table 1, below.

As defined above and described herein, L^{2′} is a covalent bond or a bivalent, saturated or partially unsaturated, straight or branched C₁₋₅ hydrocarbon chain, wherein 0-3 methylene units of L^{2′} are independently replaced by —O—, —NR—, —CRF—, —CF₂—, —C(O)—, —S—, —S(O)—, or —S(O)₂—.

In some embodiments, L^{2′} is covalent bond. In some embodiments, L^{2′} is a bivalent, saturated or partially unsaturated, straight or branched C₁₋₅ hydrocarbon chain, wherein 0-3 methylene units of L^{2′} are independently replaced by —O—, —NR—, —CRF—, —CF₂—, —C(O)—, —S—, —S(O)—, or —S(O)₂—. In some embodiments, L^{2′} is

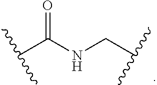 [SMILES: *CNC(*)=O]

In some embodiments, L^{2′} is

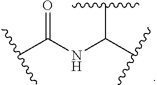 [SMILES: *C(=O)NC(*)*]

In some embodiments, L^{2′} is selected from those depicted in Table 1, below.

As defined above and described herein, R^{3′} is hydrogen or R^{A}.

In some embodiments, R^{3′} is hydrogen. In some embodiments, R^{3′} is R^{A}. In some embodiments, R^{3′} is

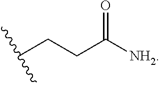 [SMILES: *CCC(N)=O]

In some embodiments, R^{3′} is selected from those depicted in Table 1, below.

As defined above and described herein, Ring M′ is an optionally substituted bivalent ring selected from phenylenyl, naphthylenyl, a 5-10 membered heteroarylenyl containing 1-4 heteroatoms independently selected from nitrogen, oxygen, or sulfur, a 5-11 membered saturated or partially unsaturated carbocyclylenyl or heterocyclylenyl with 1-4 heteroatoms independently selected from nitrogen, oxygen, or sulfur;

In some embodiments, Ring M′ is an optionally substituted phenylenyl. In some embodiments, Ring M′ is an optionally substituted naphthylenyl. In some embodiments, Ring M′ is an optionally substituted 5-10 membered heteroarylenyl containing 1-4 heteroatoms independently selected from nitrogen, oxygen, or sulfur. In some embodiments, Ring M′ is an optionally substituted 5-11 membered saturated or partially unsaturated carbocyclylenyl. In some embodiments, Ring M′ is an optionally substituted 5-11 membered saturated or partially unsaturated heterocyclylenyl with 1-4 heteroatoms independently selected from nitrogen, oxygen, or sulfur. In some embodiments, Ring M′ is

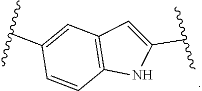 [SMILES: *c1ccc2[nH]c(*)cc2c1]

In some embodiments, Ring M′ is selected from those depicted in Table 1, below.

As defined above and described herein, Q′ is a bivalent moiety selected from —O—, —CR₂—, —CF₂—, —CFR—, —C(O)—, —OCR₂—, and —C(S)—.

In some embodiments, Q′ is —O—. In some embodiments, Q′ is —CR₂—. In some embodiments, Q′ is —OCR₂—. In some embodiments, Q′ is —CF₂—. In some embodiments, Q′ is —CFR—. In some embodiments, Q′ is —C(O)—. In some embodiments, Q′ is —C(S)—.

In some embodiments, Q′ is selected from those depicted in Table 1, below.

As defined above and described herein, R^{a1} and R^{a2} are each independently hydrogen, R^{A}, —CH₂CO₂R, or —CH₂OCO₂R.

In some embodiments, R^{a1} is hydrogen. In some embodiments, R^{a1} is R^{A}. In some embodiments, R^{a1} is —CH₂CO₂R. In some embodiments, R^{a1} is —CH₂OCO₂R. In some embodiments, R^{a2} is hydrogen. In some embodiments, R^{a2} is R^{A}. In some embodiments, R^{a2} is —CH₂CO₂R. In some embodiments, R^{a2} is —CH₂OCO₂R.

In some embodiments, R^{a1} and R^{a2} are selected from those depicted in Table 1, below.

As defined above and described herein, Y′ is an optionally substituted —(CH₂)_{y}—.

In some embodiments, Y′ is an optionally substituted-(CH₂)_{y}—. In some embodiments, Y′ is —CH₂—. In some embodiments, Y′ is

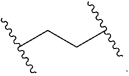 [SMILES: *CC*]

In some embodiments, Y′ is selected from those depicted in Table 1, below.

As defined above and described herein, y is 0, 1, 2, or 3.

In some embodiments, y is 0. In some embodiments, y is 1. In some embodiments, y is 2. In some embodiments, y is 3.

In some embodiments, y is selected from those depicted in Table 1, below.

As defined above and described herein, Ring W′ is an optionally substituted ring selected from a 5-9 membered saturated or partially unsaturated heterocyclyl.

In some embodiments, Ring W′ is an optionally substituted ring selected from a 5-9 membered saturated or partially unsaturated heterocyclyl. In some embodiments, Ring W′ is a 8-membered saturated heterocyclyl.

In some embodiments, Ring W′ is selected from those depicted in Table 1, below.

As defined above and described herein, Ring U′ is a ring selected from phenyl, a 5-6 membered heteroaryl containing 1-4 heteroatoms independently selected from nitrogen, oxygen, or sulfur, and a 5-7 membered saturated or partially unsaturated carbocyclyl or heterocyclyl with 1-3 heteroatoms independently selected from nitrogen, oxygen, or sulfur.

In some embodiments, Ring U′ is phenyl. In some embodiments, each Ring U′ is phenyl. In some embodiments, Ring U′ is a 5-6 membered heteroaryl containing 1-4 heteroatoms independently selected from nitrogen, oxygen, or sulfur. In some embodiments, Ring U′ is a 5-7 membered saturated or partially unsaturated carbocyclyl or heterocyclyl with 1-3 heteroatoms independently selected from nitrogen, oxygen, or sulfur.

In some embodiments, Ring U′ is selected from those depicted in Table 1, below.

As defined above and described herein, R^{u′} is hydrogen, R^{A}, halogen, —CN, —NO₂, —OR, —SR, —NR₂, —SiR₃, —S(O)₂R, —S(O)₂NR₂, —S(O)R, —C(O)R, —C(O) OR, —C(O)NR₂, —C(O)NROR, —CR₂NRC(O)R, —CR₂NRC(O)NR₂, —OC(O)R, —OC(O)NR₂, —OP(O)R², —OP(O)(OR)₂, —OP(O)(OR)NR₂, —OP(O)(NR₂)₂, —NRC(O) OR, —NRC(O)R, —NRC(O)NR₂, —NRS (O)₂R, —NP(O)R², —NRP(O)(OR)₂, —NRP(O)(OR)NR₂, —NRP(O)(NR₂)₂, or —NRS (O)₂R.

In some embodiments, R^{u′} is hydrogen. In some embodiments, R^{u′} is R^{A}. In some embodiments, R^{u′} is halogen. In some embodiments, R^{u′} is —CN. In some embodiments, R^{u′} is —NO₂. In some embodiments, R^{u′} is —OR. In some embodiments, R^{u′} is —SR. In some embodiments, R^{u′} is —NR₂. In some embodiments, R^{u′} is —SiR₃. In some embodiments, R^{u′} is —S(O)₂R. In some embodiments, R^{u′} is —S(O)₂NR₂. In some embodiments, R^{u′} is —S(O)R. In some embodiments, R^{u′} is —C(O)R. In some embodiments, R^{u′} is —C(O) OR. In some embodiments, R^{u′} is —C(O)NR₂. In some embodiments, R^{u′} is —C(O)NROR. In some embodiments, R^{u′} is —CR₂NRC(O)R. In some embodiments, R^{u′} is —CR₂NRC(O)NR₂. In some embodiments, R^{u′} is —OC(O)R. In some embodiments, R^{u′} is —OC(O)NR₂. In some embodiments, R^{u′} is —OP(O)R². In some embodiments, R^{u′} is —OP(O)(OR)₂. In some embodiments, R^{u′} is —OP(O)(OR)NR₂. In some embodiments, R^{u′} is —OP(O)(NR₂)₂. In some embodiments, R^{u′} is —NRC(O) OR. In some embodiments, R^{u′} is —NRC(O)R. In some embodiments, R^{u′} is —NRC(O)NR₂. In some embodiments, R^{u′} is —NRS (O)₂R. In some embodiments, R^{u′} is —NP(O)R². In some embodiments, R^{u′} is —NRP(O)(OR)₂. In some embodiments, R^{u′} is —NRP(O)(OR)NR₂. In some embodiments, R^{u′} is —NRP(O)(NR₂)₂. In some embodiments, R^{u′} is —NRS (O)₂R. In some embodiments, R^{u′} is -iPr. In some embodiments, R^{u′} is —S(O)₂iPr. In some embodiments, R^{u′} is —S(O)₂CH₃.

In some embodiments, R^{u′} is selected from those depicted in Table 1, below.

As defined above and described herein, u is 0, 1, 2, 3, or 4.

In some embodiments, u is 0. In some embodiments, u is 1. In some embodiments, u is 2. In some embodiments, u is 3. In some embodiments, u is 4.

In some embodiments, u is selected from those depicted in Table 1, below.

As defined above and described herein, Ring Z′ is a bivalent ring selected from phenylenyl, a 4-7 membered saturated or partially unsaturated carbocyclylenyl or heterocyclylenyl having 1-2 heteroatoms independently selected from nitrogen, oxygen, and sulfur, and a 5-6 membered heteroarylenyl having 1-4 heteroatoms independently selected from nitrogen, oxygen, and sulfur.

In some embodiments, Ring Z′ is phenylenyl. In some embodiments, Ring Z′ is a 4-7 membered saturated or partially unsaturated carbocyclylenyl. In some embodiments, Ring Z′ is a heterocyclylenyl having 1-2 heteroatoms independently selected from nitrogen, oxygen, and sulfur. In some embodiments, Ring Z′ is a 5-6 membered heteroarylenyl having 1-4 heteroatoms independently

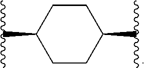 [SMILES: *[C@H]1CC[C@@H](*)CC1]

selected from nitrogen, oxygen, and sulfur. In some embodiments, Ring Z′ is

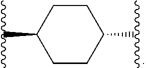 [SMILES: *[C@H]1CC[C@H](*)CC1]

In some embodiments, Ring Z′ is

In some embodiments, Ring Z′ is selected from those depicted in Table 1, below.

As defined above and described herein, R^{z′} is hydrogen, R^{A}, halogen, —CN, —NO₂, —OR, —SR, —NR₂, —SiR₃, —S(O)₂R, —S(O)₂NR₂, —S(O)R, —C(O)R, —C(O) OR, —C(O)NR₂, —C(O)NROR, —CR₂NRC(O)R, —CR₂NRC(O)NR₂, —OC(O)R, —OC(O)NR₂, —OP(O)R², —OP(O)(OR)₂, —OP(O)(OR)NR₂, —OP(O)(NR₂)₂, —NRC(O) OR, —NRC(O)R, —NRC(O)NR₂, —NRS (O)₂R, —NP(O)R², —NRP(O)(OR)₂, —NRP(O)(OR)NR₂, —NRP(O)(NR₂)₂, or —NRS (O)₂R.

In some embodiments, R^{z′} is hydrogen. In some embodiments, R^{z′} is R^{A}. In some embodiments, R^{z′} is halogen. In some embodiments, R^{2′} is —CN. In some embodiments, R^{z′} is —NO₂. In some embodiments, R^{z′} is —OR. In some embodiments, R^{z′} is —SR. In some embodiments, R^{z′} is —NR₂. In some embodiments, R^{z′} is —SiR₃. In some embodiments, R^{z′} is —S(O)₂R. In some embodiments, R^{z′} is —S(O)₂NR₂. In some embodiments, R^{z′} is —S(O)R, —C(O)R. In some embodiments, R^{z′} is —C(O) OR. In some embodiments, R^{2′} is —C(O)NR₂. In some embodiments, R^{z′} is —C(O)NROR. In some embodiments, R^{z′} is —CR₂NRC(O)R. In some embodiments, R^{z′} is —CR₂NRC(O)NR₂. In some embodiments, Rz is —OC(O)R. In some embodiments, R^{z′} is —OC(O)NR₂. In some embodiments, R^{z′} is —OP(O)R². In some embodiments, R^{z′} is —OP(O)(OR)₂. In some embodiments, R^{z′} is —OP(O)(OR)NR₂. In some embodiments, R^{z′} is —OP(O)(NR₂)₂. In some embodiments, R^{z′} is —NRC(O) OR. In some embodiments, R^{z′} is —NRC(O)R. In some embodiments, R^{z′} is —NRC(O)NR₂. In some embodiments, R^{z′} is —NRS (O)₂R. In some embodiments, R^{z′} is —NP(O)R². In some embodiments, R^{z′} is —NRP(O)(OR)₂. In some embodiments, R^{z′} is —NRP(O)(OR)NR₂. In some embodiments, R^{z′} is —NRP(O)(NR₂)₂. In some embodiments, R^{z′} is —NRS (O)₂R. In some embodiments, R^{2′} is —CH₃. In some embodiments, R^{z′} is —C₁. In some embodiments, R^{z′} is —F.

In some embodiments, R^{z′} is selected from those depicted in Table 1, below.

As defined above and described herein, z is 0, 1, 2, 3 or 4.

In some embodiments, z is 0. In some embodiments, z is 1. In some embodiments, z is 2. In some embodiments, z is 3. In some embodiments, z is 4.

In some embodiments, z is selected from those depicted in Table 1, below.

As defined above and described herein, n is 0 or 1.

In some embodiments, n is 0. In some embodiments, n is 1.

In some embodiments, n is selected from those depicted in Table 1, below.

As defined above and described herein, n′ is 1 or 2.

In some embodiments, n′ is 1. In some embodiments, n′ is 2.

In some embodiments, n′ is selected from those depicted in Table 1, below.

In some embodiments, the present invention provides a compound of formula I-bb, wherein KBM is a compound of formula I-a, thereby forming a compound of formula I-bb-1:

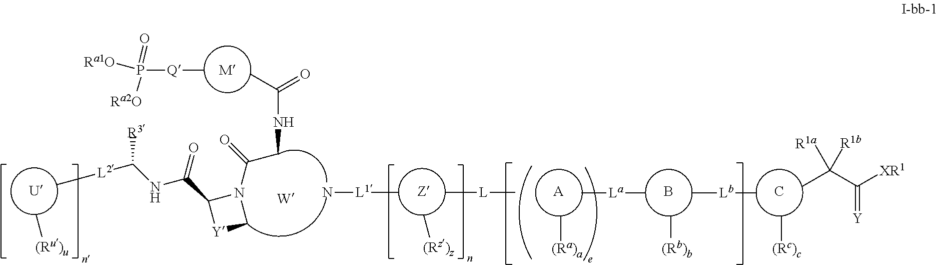

or a pharmaceutically acceptable salt thereof, wherein each of the variables is as defined and described herein, both independently and in combination.

In some embodiments, the present invention provides a compound of formula I-bb-1, wherein L¹′ is a covalent bond, n is 0, e is 1, and L′ is —C(O)NH— where attachment to L is as shown, thereby forming a compound of formula I-bb-2:

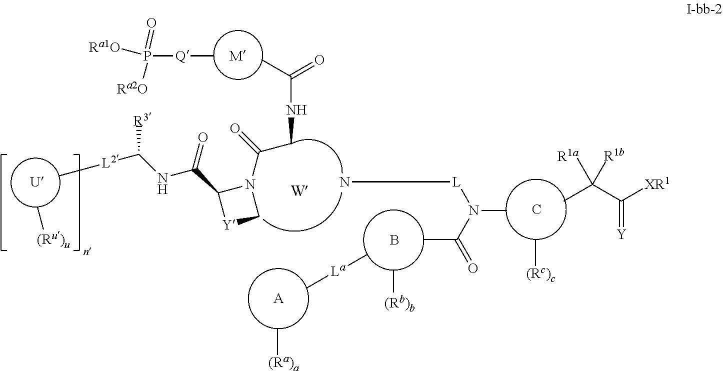

or a pharmaceutically acceptable salt thereof, wherein each of the variables is as defined and described herein, both independently and in combination.

In some embodiments, the present invention provides a compound of formula I-bb-1, wherein L¹′ is a covalent bond, n is 0, e is 1, R^{1a} and R^{1b} are hydrogen, X is —O—, Y is O, and L^{b} is —C(O)NH— where attachment to L is as shown, thereby forming a compound of formula I-bb-3:

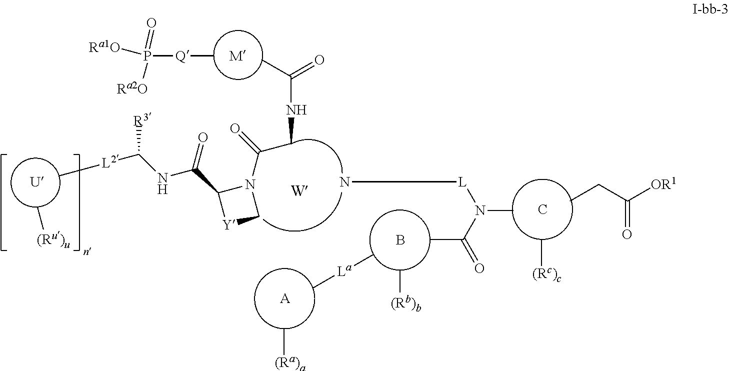

or a pharmaceutically acceptable salt thereof, wherein each of the variables is as defined and described herein, both independently and in combination.

In some embodiments, the present invention provides a compound of formula I-bb-1, wherein L¹′ is a covalent bond, n is 0, e is 1, R^{1a} and R^{1b} are hydrogen, X is —O—, Y is O, Ring C is 2-pyridonyl, and L^{b} is —C(O)NH— where attachment to L is as shown, thereby forming a compound of formula I-bb-4:

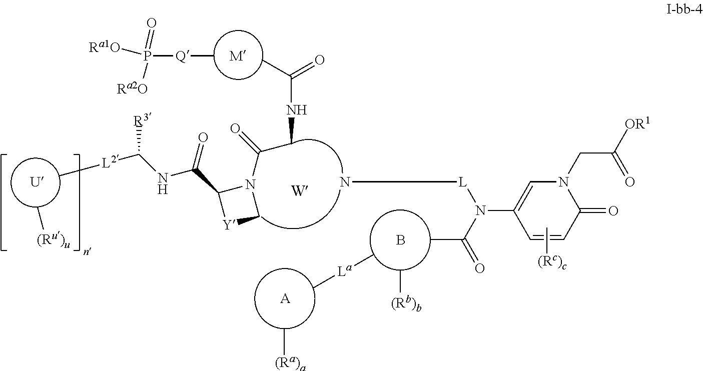

or a pharmaceutically acceptable salt thereof, wherein each of the variables is as defined and described herein, both independently and in combination.

In some embodiments, TBM is a BET/BRD4 binding moiety of formula I-cc:

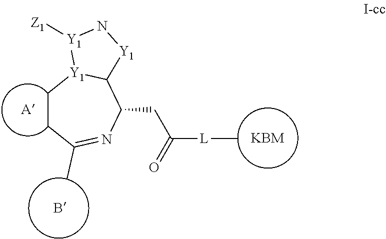

or a pharmaceutically acceptable salt thereof, wherein L and KBM are as defined above and described in embodiments herein, and wherein:
Ring A′ and Ring B′ are independently an aromatic ring, a heteroaromatic ring, a 5-membered carbocyclyl, a 6-membered carbocyclyl, a 5-membered heterocyclyl, a 6-membered heterocyclyl, a thiophene, a pyrrole, a pyrazole, a pyridine, a pyrimidine, a pyrazine, optionally substituted by alkyl, alkoxy, halogen, nitrile or another aromatic or heteroaromatic ring;
each Y₁, Y₂, Y₃ and Y₄ can independently be carbon, nitrogen or oxygen to form a fused 5-membered aromatic ring such as triazole or isoxazole; and
Z¹ is methyl, or lower alkyl group.

As defined above and described herein, Ring A′ and Ring B′ are independently an aromatic ring, a heteroaromatic ring, a 5-membered carbocyclyl, a 6-membered carbocyclyl, a 5-membered heterocyclyl, a 6-membered heterocyclyl, a thiophene, a pyrrole, a pyrazole, a pyridine, a pyrimidine, a pyrazine, optionally substituted by alkyl, alkoxy, halogen, nitrile or another aromatic or heteroaromatic ring.

In some embodiments, Ring A′ is a 1,2-fused aromatic ring. In some embodiments, Ring A′ is 1,2-fused phenyl or benzo. In some embodiments, Ring A′ is a 1,2-fused heteroaromatic ring. In some embodiments, Ring A′ is a 1,2-fused 5-membered carbocyclyl. In some embodiments, Ring A′ is a 1,2-fused 6-membered carbocyclyl. In some embodiments, Ring A′ is a 1,2-fused 5-membered heterocyclyl. In some embodiments, Ring A′ is a 1,2-fused 6-membered heterocyclyl. In some embodiments, Ring A′ is 1,2-fused thiophene. In some embodiments, Ring A′ is 1,2-fused pyrrole. In some embodiments, Ring A′ is 1,2-fused pyrazole. In some embodiments, Ring A′ is 1,2-fused pyridine. In some embodiments, Ring A′ is 1,2-fused pyrimidine. In some embodiments, Ring A′ is 1,2-fused pyrazine. In some embodiments, Ring A′ is substituted by alkyl, alkoxy, halogen, nitrile or another aromatic or heteroaromatic ring.

In some embodiments, Ring B′ is an aromatic ring. In some embodiments, Ring B′ is phenyl. In some embodiments, Ring B′ is a heteroaromatic ring. In some embodiments, Ring B′ is a 5-membered carbocyclyl. In some embodiments, Ring B′ is a 6-membered carbocyclyl. In some embodiments, Ring B′ is a 5-membered heterocyclyl. In some embodiments, Ring B′ is a 6-membered heterocyclyl. In some embodiments, Ring B′ is thiophene. In some embodiments, Ring B′ is pyrrole. In some embodiments, Ring B′ is pyrazole. In some embodiments, Ring B′ is pyridine. In some embodiments, Ring B′ is pyrimidine. In some embodiments, Ring B′ is pyrazine. In some embodiments, Ring B′ is substituted by alkyl, alkoxy, halogen, nitrile or another aromatic or heteroaromatic ring.

In some embodiments, Ring A′ and Ring B′ are selected from those depicted in Table 1, below.

As defined above and described herein, Y₁, Y₂, Y₃ and Y₄ can be carbon, nitrogen or oxygen to form a fused 5-membered aromatic ring such as triazole or isoxazole.

In some embodiments, Y₁ is carbon. In some embodiments, Y₁ is nitrogen. In some embodiments, Y₁ is oxygen. In some embodiments, Y₂ is carbon. In some embodiments, Y₂ is nitrogen. In some embodiments, Y₂ is oxygen. In some embodiments, Y₃ is carbon. In some embodiments, Y₃ is nitrogen. In some embodiments, Y₃ is oxygen. In some embodiments, Y₄ is carbon. In some embodiments, Y₄ is nitrogen. In some embodiments, Y₄ is oxygen.

In some embodiments, Y₁, Y₂, Y₃ and Y₄ are selected from those depicted in Table 1, below.

As defined above and described herein, Z is methyl, or lower alkyl group.

In some embodiments, Z₁ is methyl. In some embodiments, Z₁ is a lower alkyl group.

In some embodiments, the present invention provides a compound of formula I-cc, wherein KBM is a compound of formula I-a′, thereby forming a compound of formula I-cc-1:

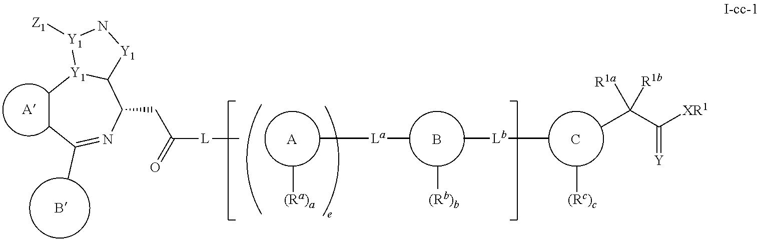

or a pharmaceutically acceptable salt thereof, wherein each of the variables is as defined and described herein, both independently and in combination.

In some embodiments, the present invention provides a compound of formula I-cc-1, wherein e is 1 and L^{b} is —C(O)NH— where attachment to L is as shown, thereby forming a compound of formula I-cc-2:

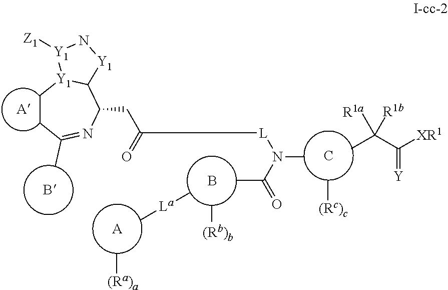

or a pharmaceutically acceptable salt thereof, wherein each of the variables is as defined and described herein, both independently and in combination.

In some embodiments, the present invention provides a compound of formula I-cc-1, wherein e is 1, R^{1a} and R^{1b} are hydrogen, X is —O—, Y is O, and L^{b} is —C(O)NH— where attachment to L is as shown, thereby forming a compound of formula I-cc-3:

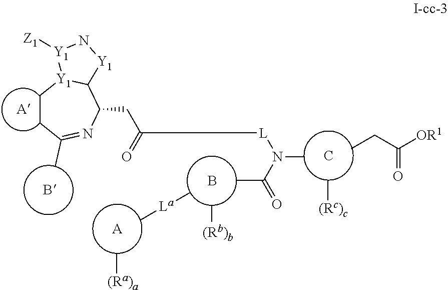

or a pharmaceutically acceptable salt thereof, wherein each of the variables is as defined and described herein, both independently and in combination.

In some embodiments, the present invention provides a compound of formula I-cc-1, wherein e is 1, R^{1a} and R^{1b} are hydrogen, X is —O—, Y is O, Ring C is 2-pyridonyl, and L^{b} is —C(O)NH— where attachment to L is as shown, thereby forming a compound of formula I-cc-4:

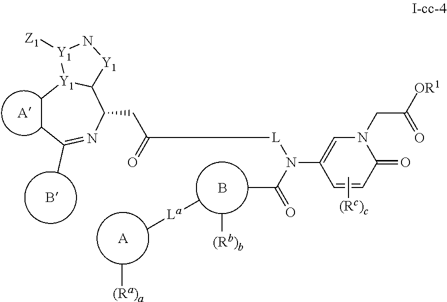

or a pharmaceutically acceptable salt thereof, wherein each of the variables is as defined and described herein, both independently and in combination.

In some embodiments, TBM is a BRD ligand selected from

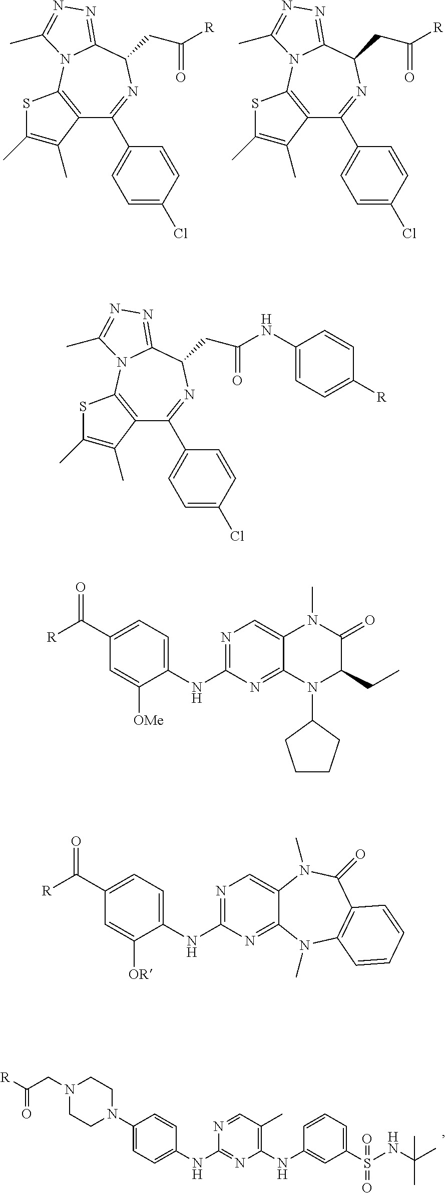

wherein R denotes attachment to

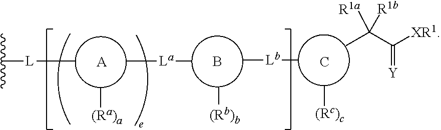

In some embodiments, TBM is a CREBBP ligand selected from

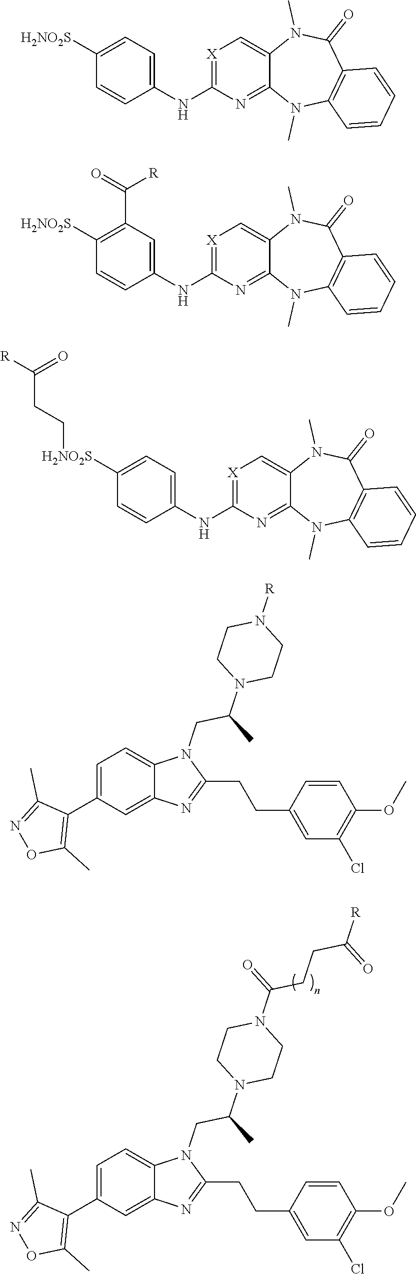

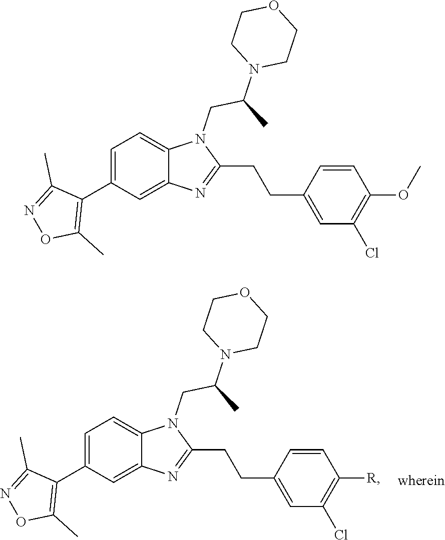

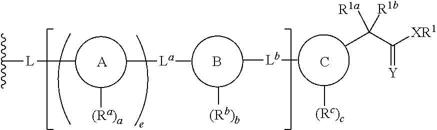

is attached to R or a modifiable carbon, oxygen, nitrogen or sulfur atom, X is N or C, and n is 0 to 8.

In some embodiments, TBM is a TRIM24/BRPF1 ligand selected from

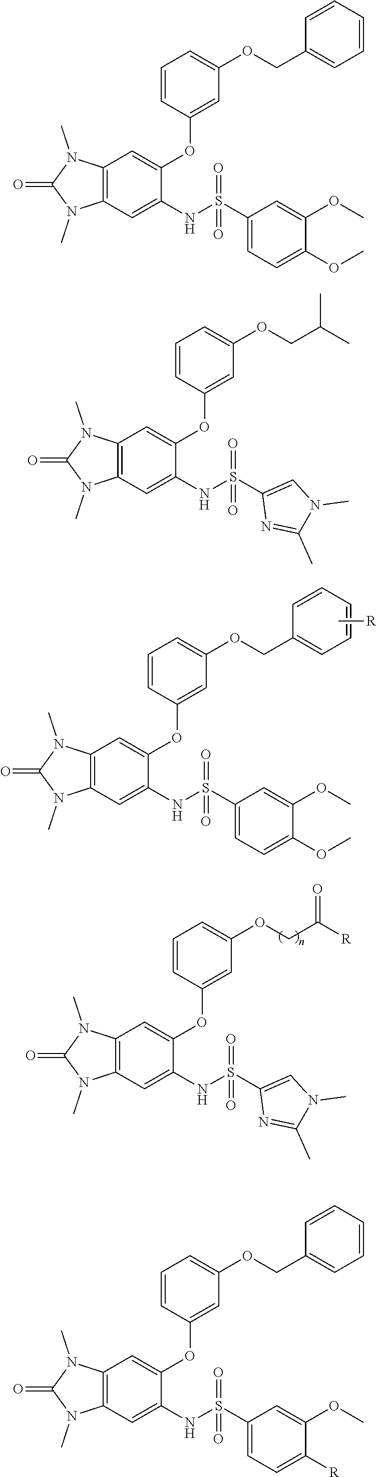

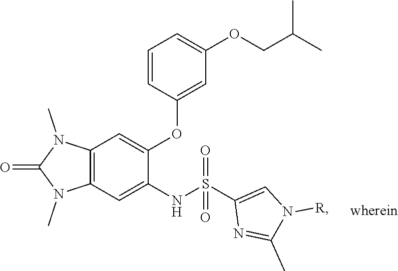

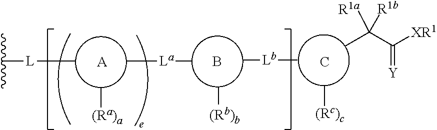

is attached to R or a modifiable carbon, oxygen, nitrogen or sulfur atom, and n is 0 to 8.

In some embodiments, TBM is a glucocorticoid receptor ligand selected from

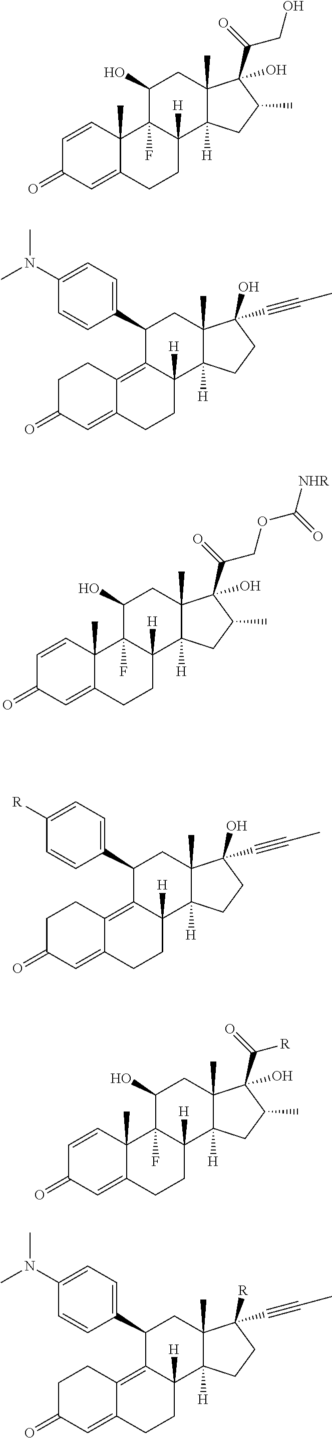

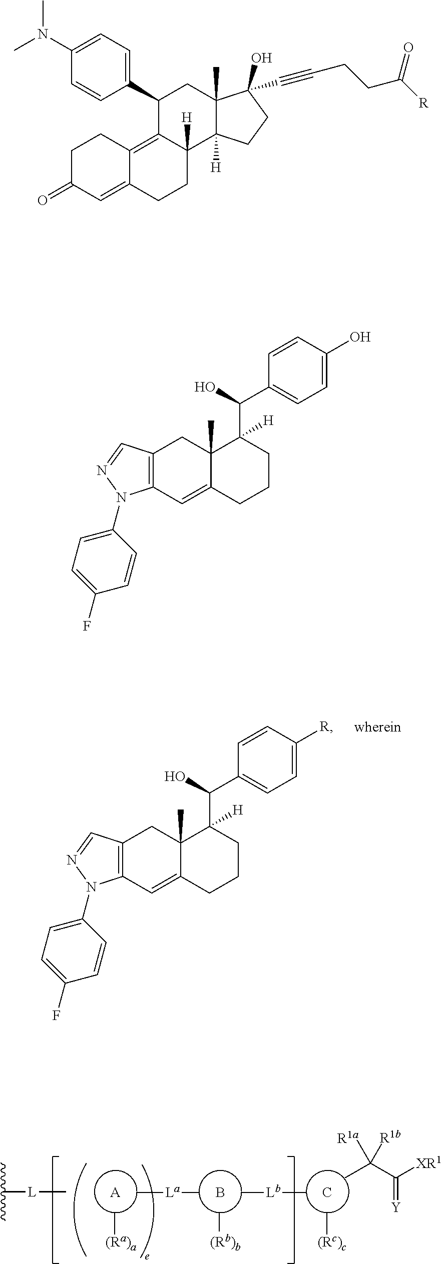

is attached to R or a modifiable carbon, oxygen, nitrogen or sulfur atom.

In some embodiments, TBM is an estrogen/androgen receptor ligand selected from

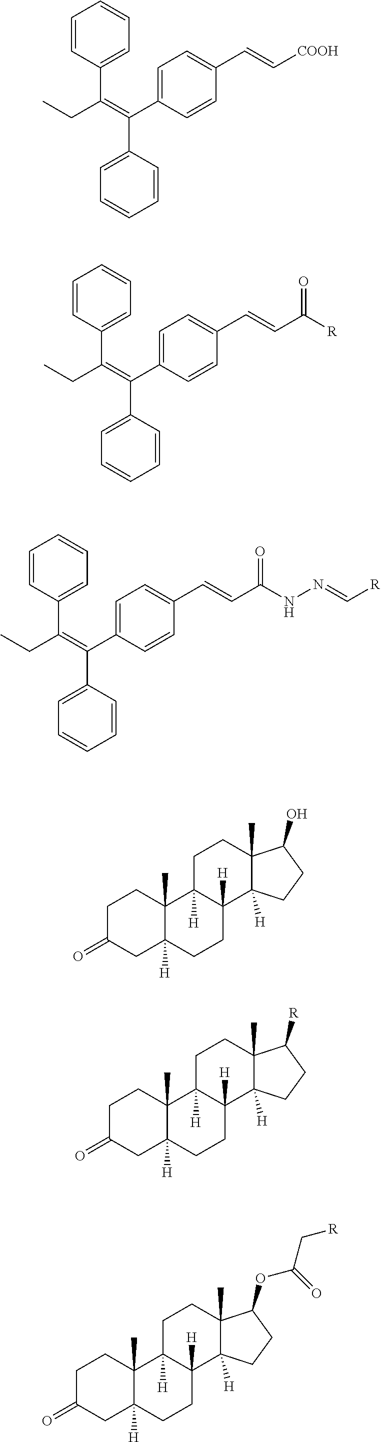

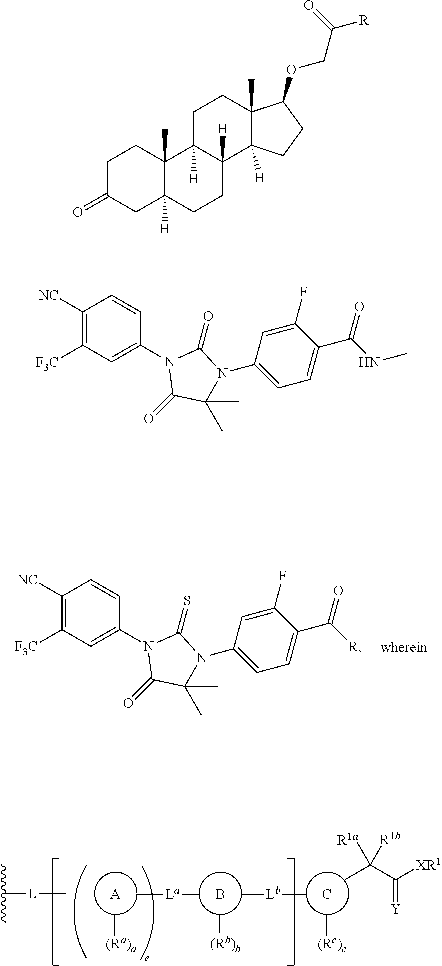

is attached to R or a modifiable carbon, oxygen, nitrogen or sulfur atom, X is N or C, and n is 0 to 8.

In some embodiments, TBM is a DOTIL ligand selected from

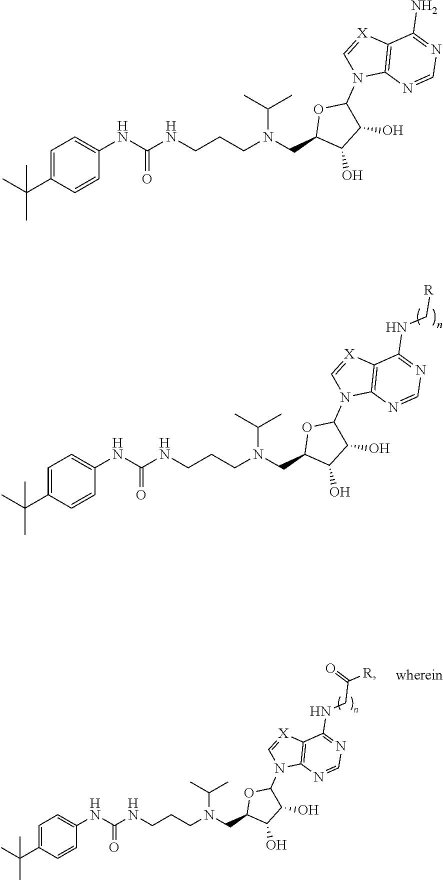

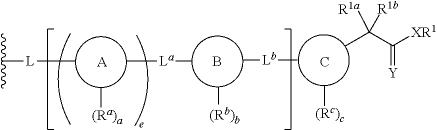

is attached to R or a modifiable carbon, oxygen, nitrogen or sulfur atom.

In some embodiments, TBM is a BRAF ligand selected from

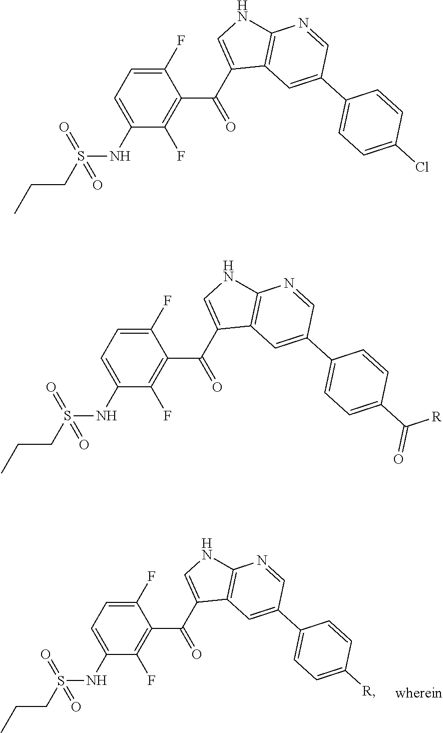

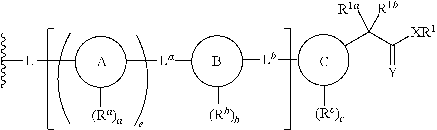

is attached to R or a modifiable carbon, oxygen, nitrogen or sulfur atom.

In some embodiments, TBM is a Ras ligand selected from

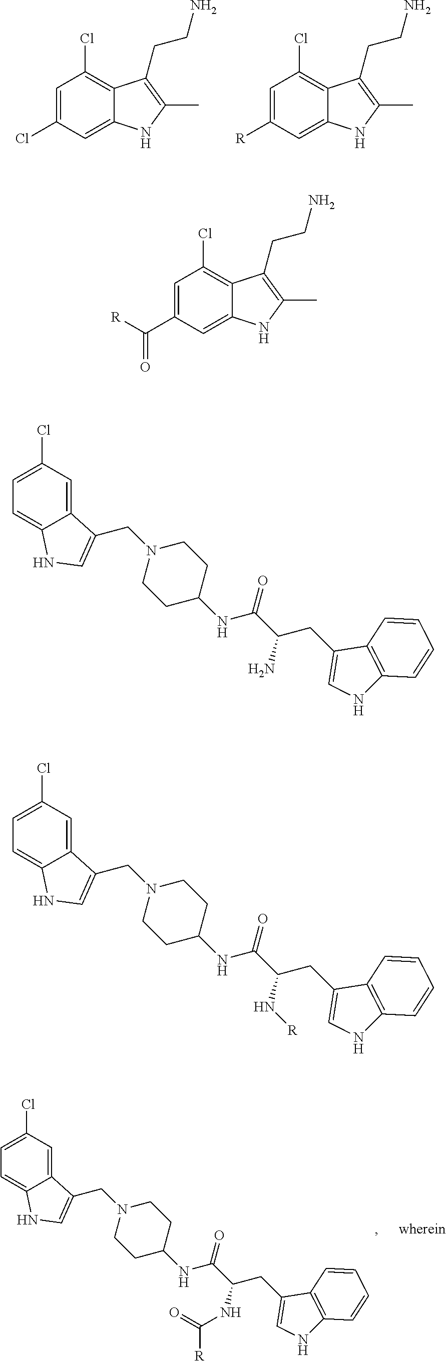

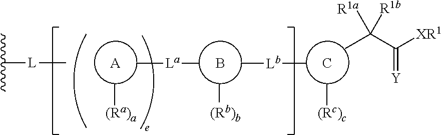

is attached to R or a modifiable carbon, oxygen, nitrogen or sulfur atom.

In some embodiments, TBM is a RasG12C ligand selected from

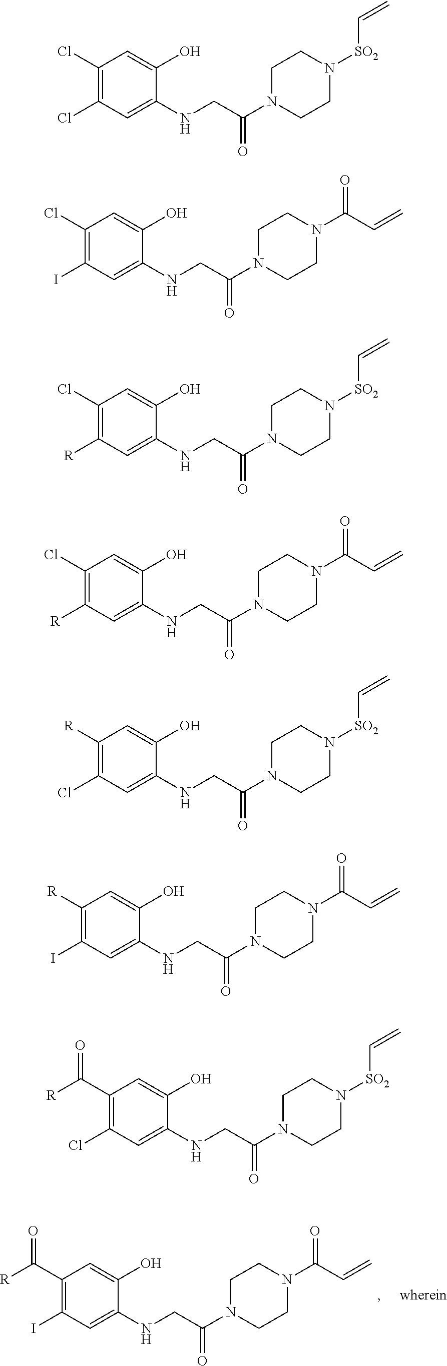

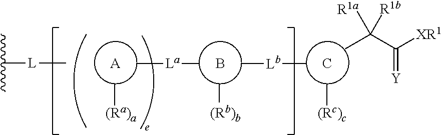

is attached to R or a modifiable carbon, oxygen, nitrogen or sulfur atom.

In some embodiments, TBM is a Her3 ligand selected from

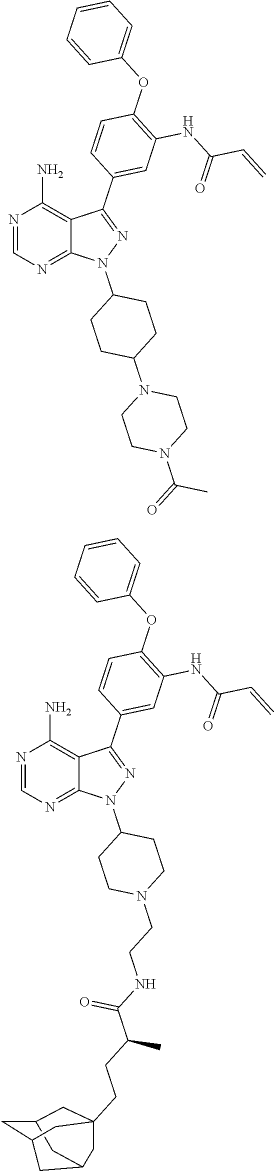

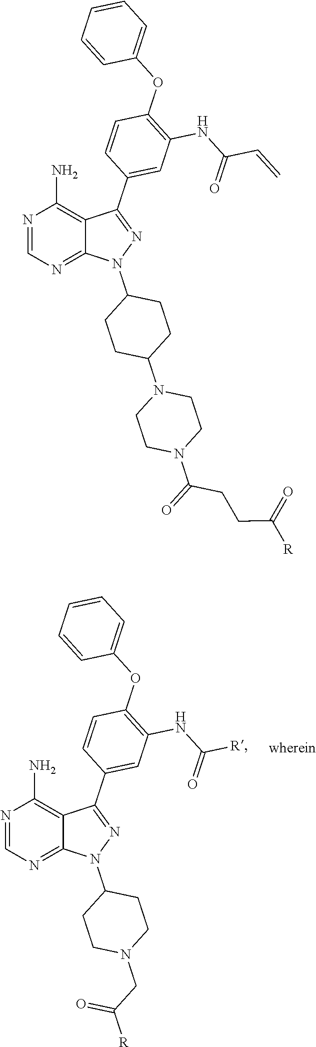

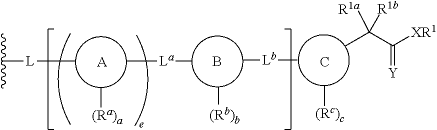

is attached to R or a modifiable carbon, oxygen, nitrogen or sulfur atom, and R′ is —CH₂CH₃ or —CH═CH₂.

In some embodiments, TBM is a Bcl-2/Bcl-XL ligand selected from

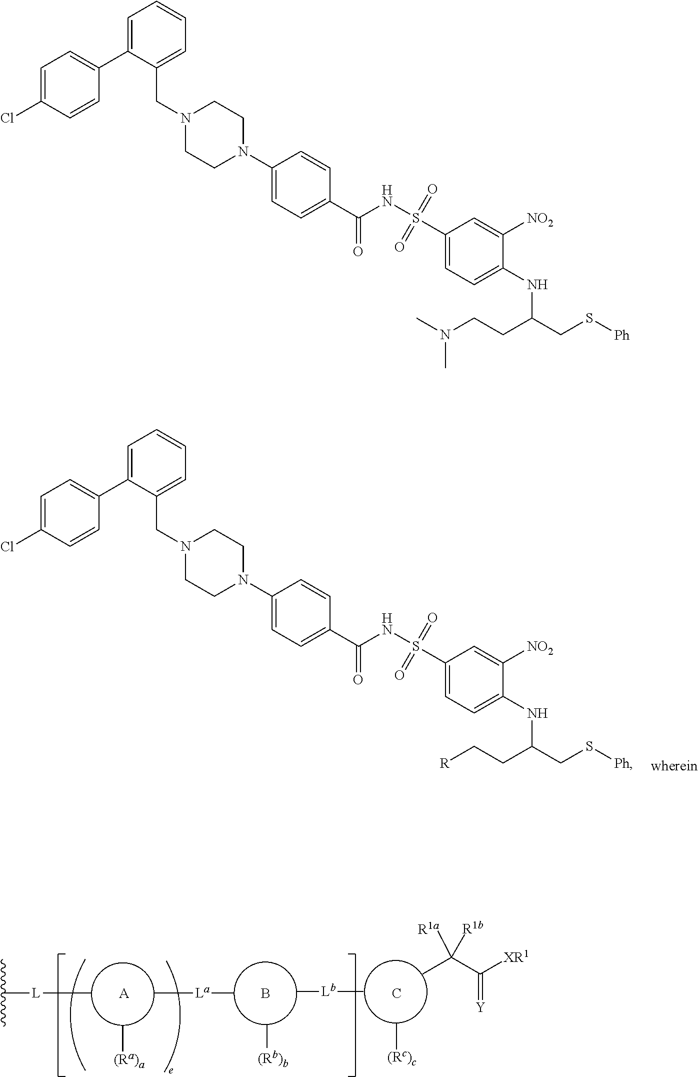

is attached to R or a modifiable carbon, oxygen, nitrogen or sulfur atom.

In some embodiments, TBM is an HDAC ligand selected from

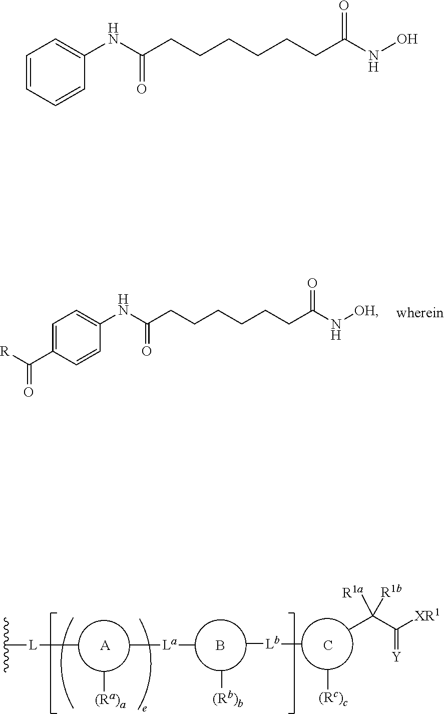

is attached to R or a modifiable carbon, oxygen, nitrogen or sulfur atom.

In some embodiments, TBM is a PPAR-gamma ligand selected from

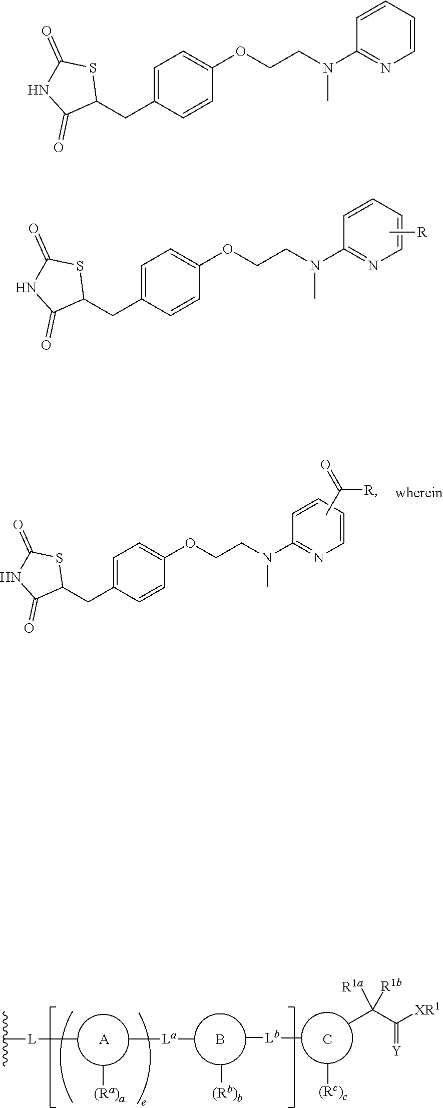

is attached to R or a modifiable carbon, oxygen, nitrogen or sulfur atom.

In some embodiments, TBM is selected from

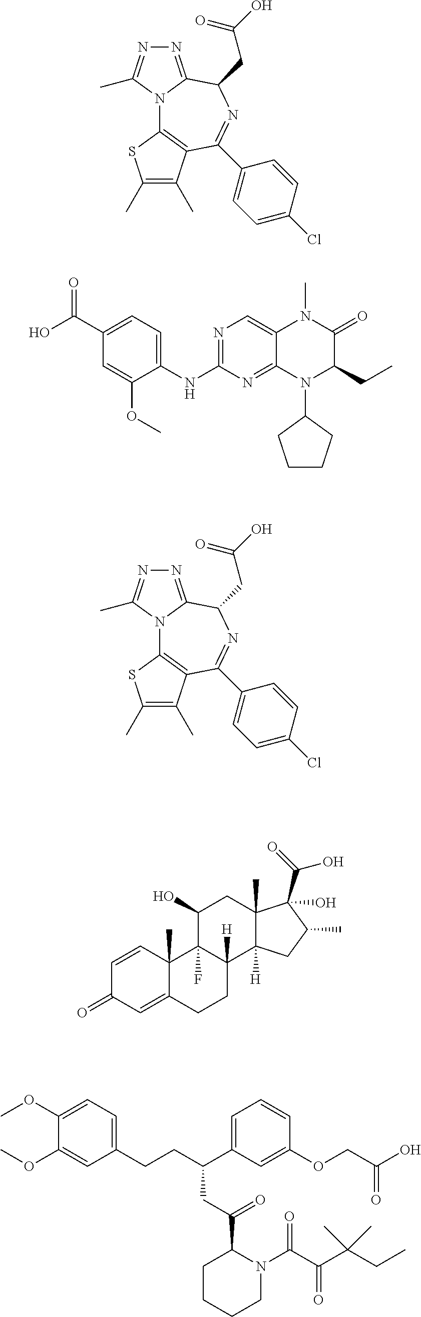

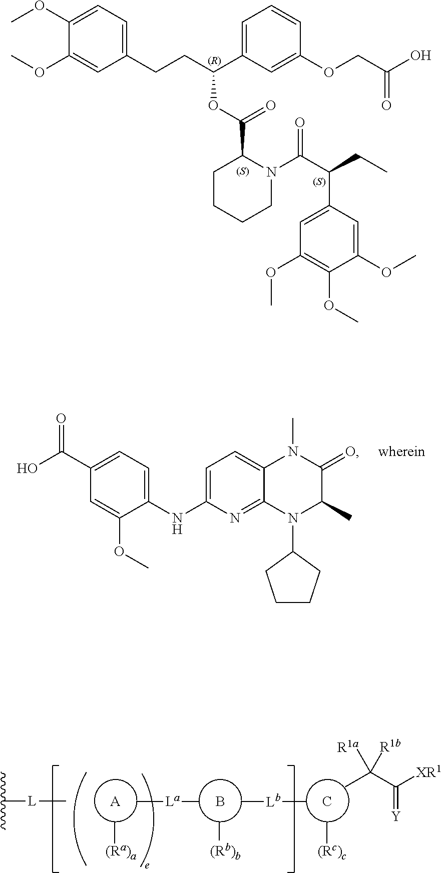

is attached to a modifiable carbon, oxygen, nitrogen or sulfur atom.

In some embodiments, TBM is an Abl, KRAS, SHP2, cRAF, or PRMT5 ligand that are selected from the following non-limiting examples:

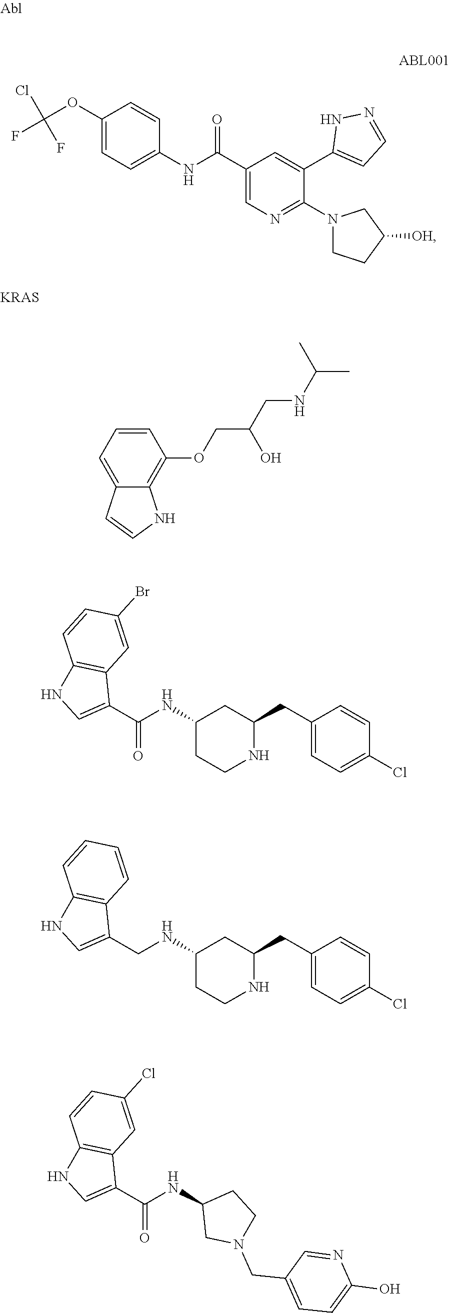 [SMILES: O=C(Nc1ccc(OC(F)(F)Cl)cc1)c1cnc(N2CC[C@@H](O)C2)c(-c2ccn[nH]2)c1 ; CC(C)NCC(O)COc1cccc2cc[nH]c12 ; O=C(N[C@H]1CCN[C@H](Cc2ccc(Cl)cc2)C1)c1c[nH]c2ccc(Br)cc12 ; Clc1ccc(C[C@@H]2C[C@@H](NCc3c[nH]c4ccccc34)CCN2)cc1 ; O=C(N[C@H]1CCN(Cc2ccc(O)nc2)C1)c1c[nH]c2ccc(Cl)cc12]

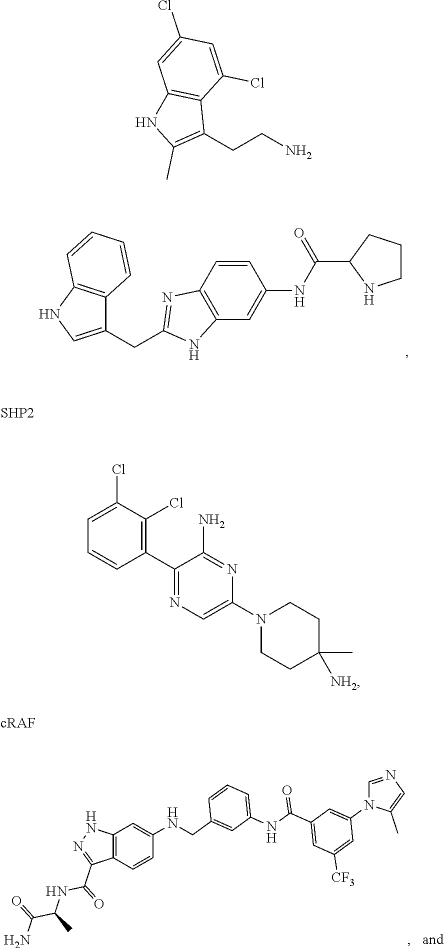 [SMILES: Cc1[nH]c2cc(Cl)cc(Cl)c2c1CCN ; O=C(Nc1ccc2nc(Cc3c[nH]c4ccccc34)[nH]c2c1)C1CCCN1 ; "SHP2" ; CC1(N)CCN(c2cnc(-c3cccc(Cl)c3Cl)c(N)n2)CC1 ; Cc1cncn1-c1cc(C(=O)Nc2cccc(CNc3ccc4c(C(=O)N[C@@H](C)C(N)=O)n[nH]c4c3)c2)cc(C(F)(F)F)c1]

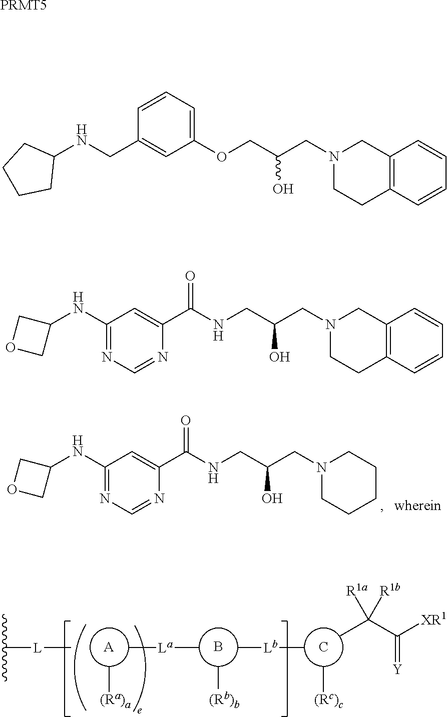

is attached to a modifiable carbon, oxygen, nitrogen or sulfur atom.

In some embodiments, TBM is a EZH2 ligand selected from

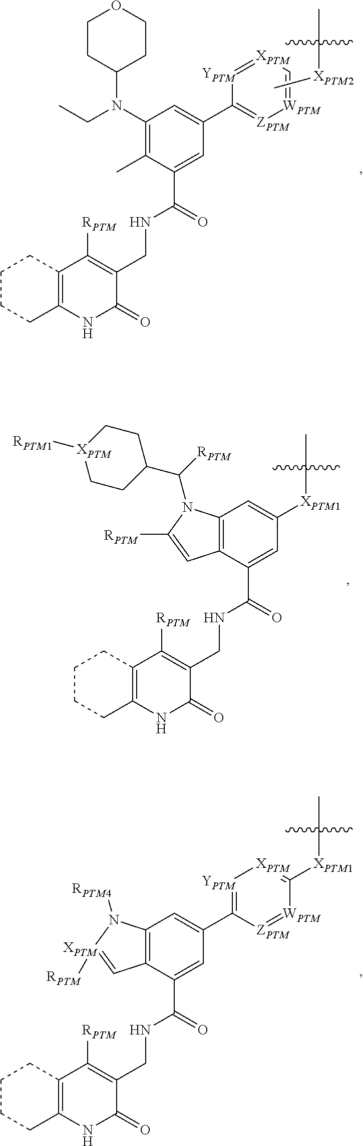

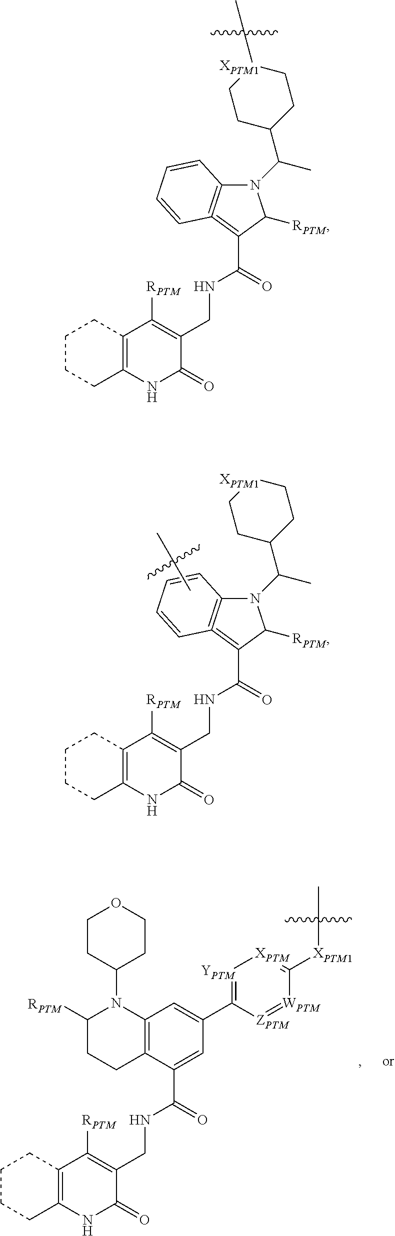

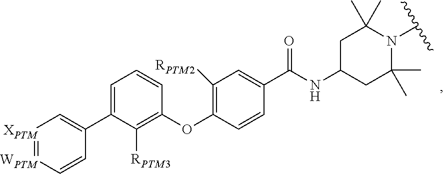

wherein [symbol] denotes attachment to

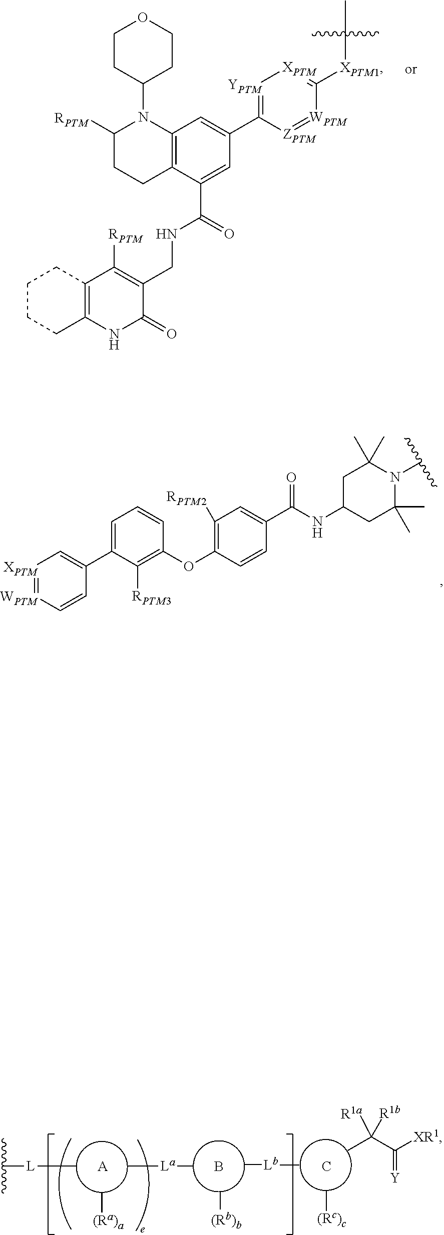

and wherein each of variables RP™ (1-4), WPTM, XPTM, YPTM, and ZP™ is as defined in WO 2018/119357 and US 2018/0177750, the entirety of each of which is herein incorporated by reference.

In some embodiments, TBM is a FLT3 ligand selected from denotes attachment to

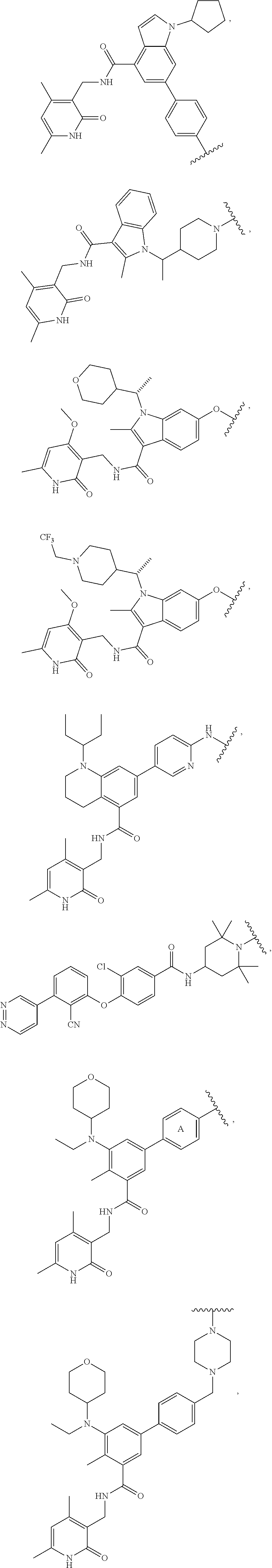

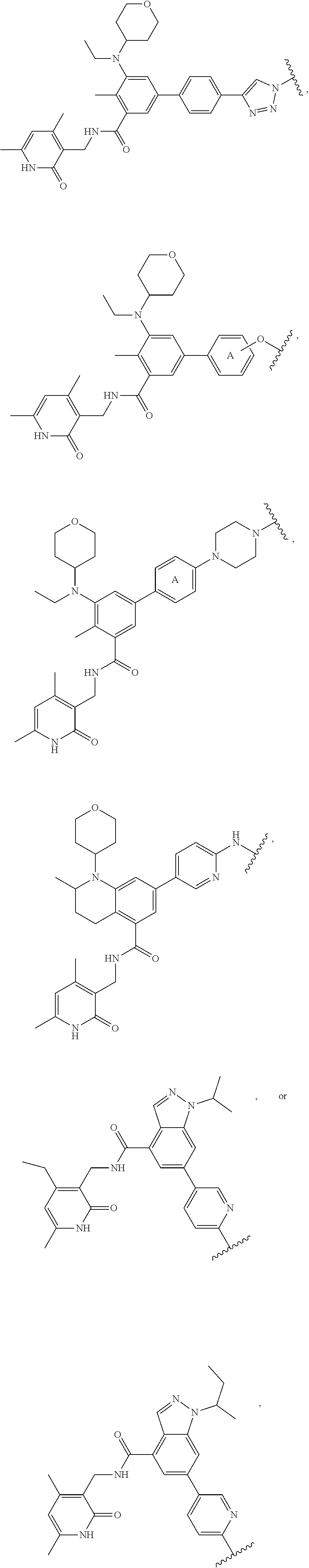

wherein [symbol] denotes attachment to

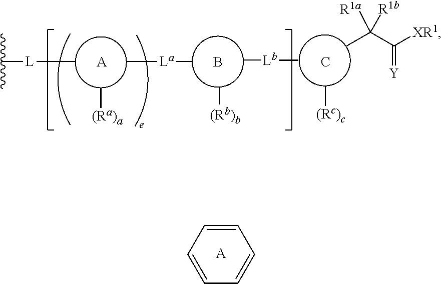

### may be N-substituted.

In some embodiments, a TBM moiety is selected from

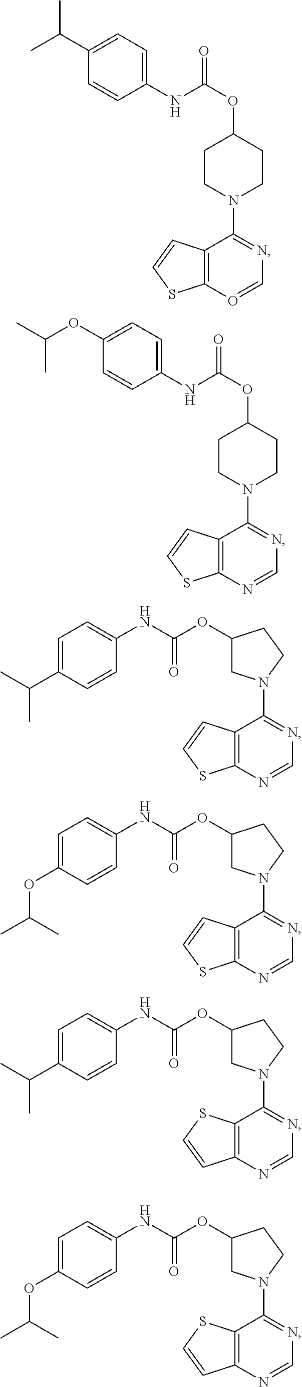

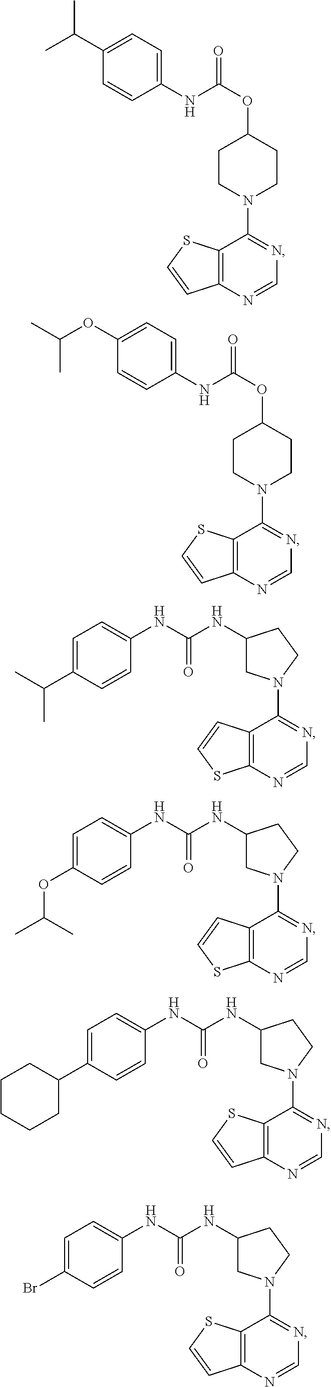

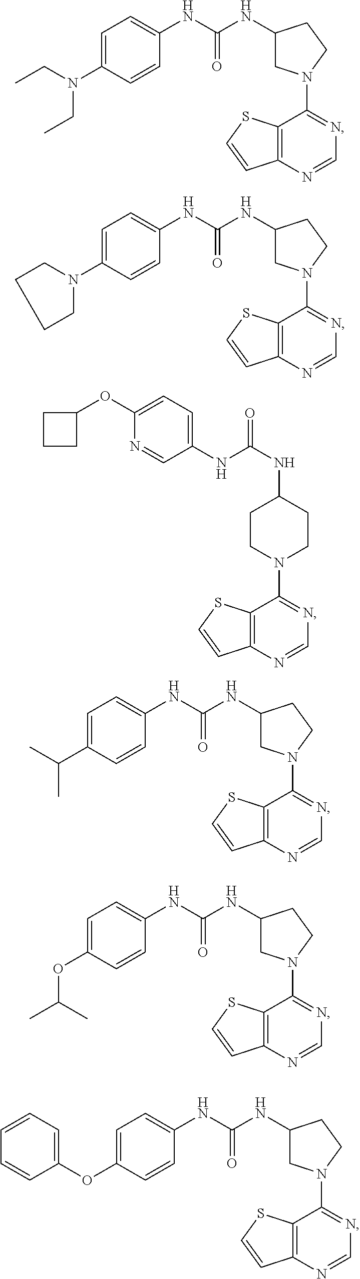

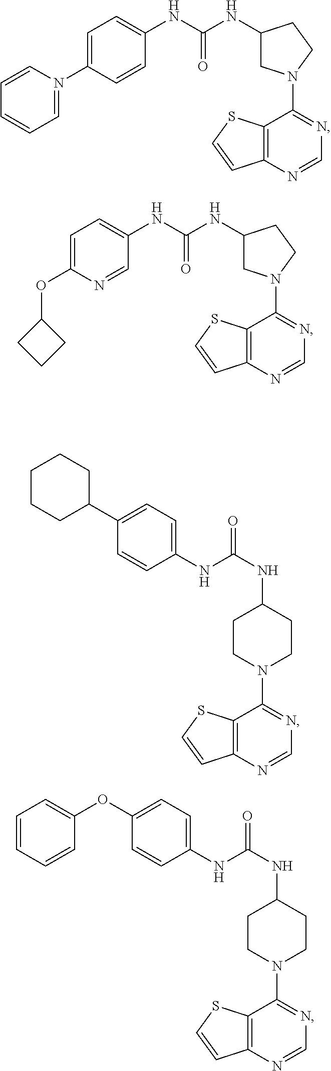

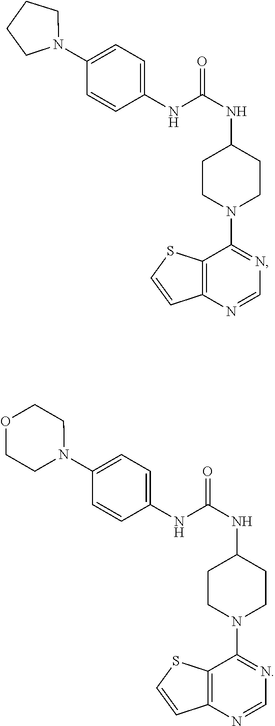 [SMILES: O=C(Nc1ccc(N2CCCC2)cc1)NC1CCN(c2ncnc3ccsc23)CC1 ; O=C(Nc1ccc(N2CCOCC2)cc1)NC1CCN(c2ncnc3ccsc23)CC1]

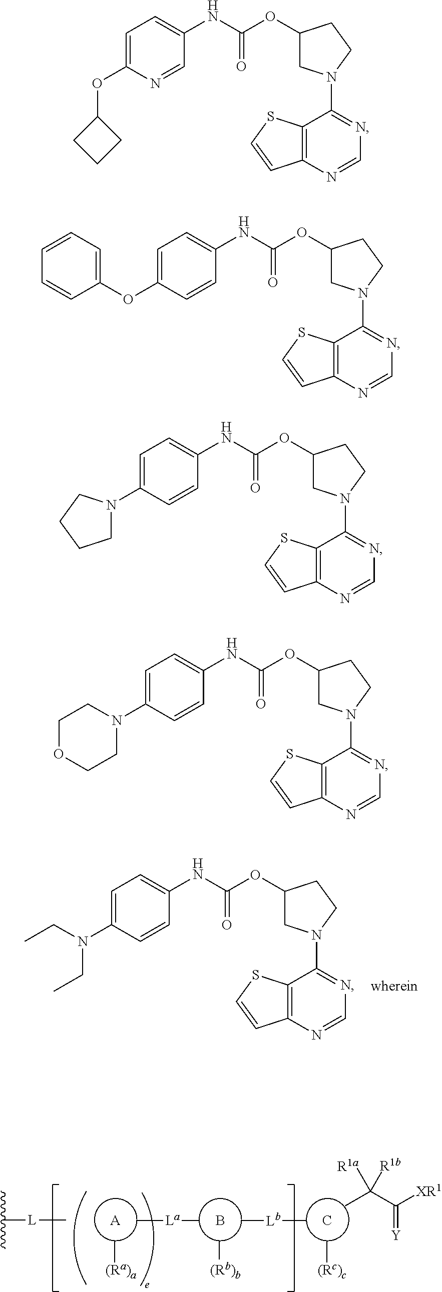

is attached to a modifiable carbon, oxygen, nitrogen or sulfur atom.

In some embodiments, a TBM moiety is a RAF ligand selected from

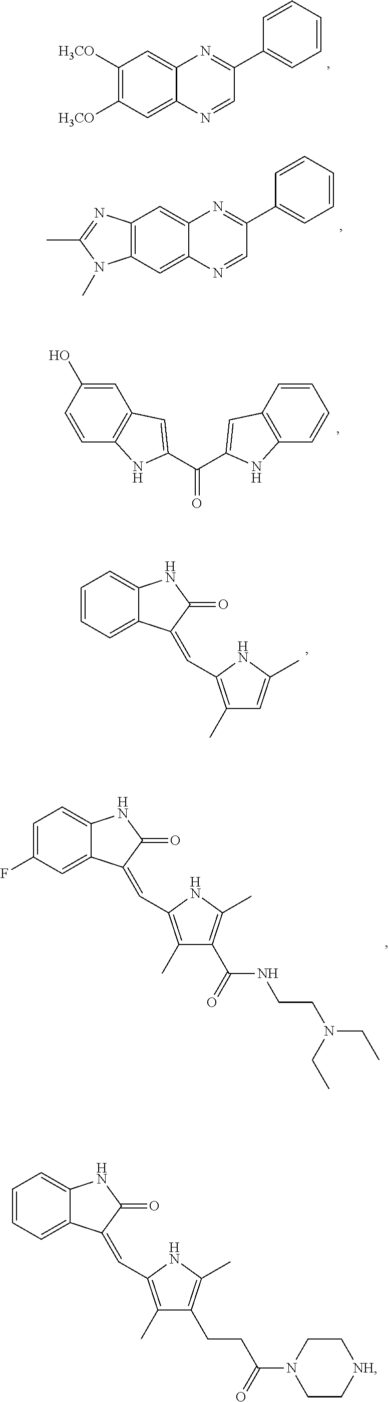 [SMILES: COc1cc2ncc(-c3ccccc3)nc2cc1OC ; Cc1nc2cc3nc(-c4ccccc4)cnc3cc2n1C ; O=C(c1cc2ccccc2[nH]1)c1cc2cc(O)ccc2[nH]1 ; Cc1cc(C)c(/C=C2\C(=O)Nc3ccccc32)[nH]1 ; CCN(CC)CCNC(=O)c1c(C)[nH]c(/C=C2\C(=O)Nc3ccc(F)cc32)c1C ; Cc1[nH]c(/C=C2\C(=O)Nc3ccccc32)c(C)c1CCC(=O)N1CCNCC1]

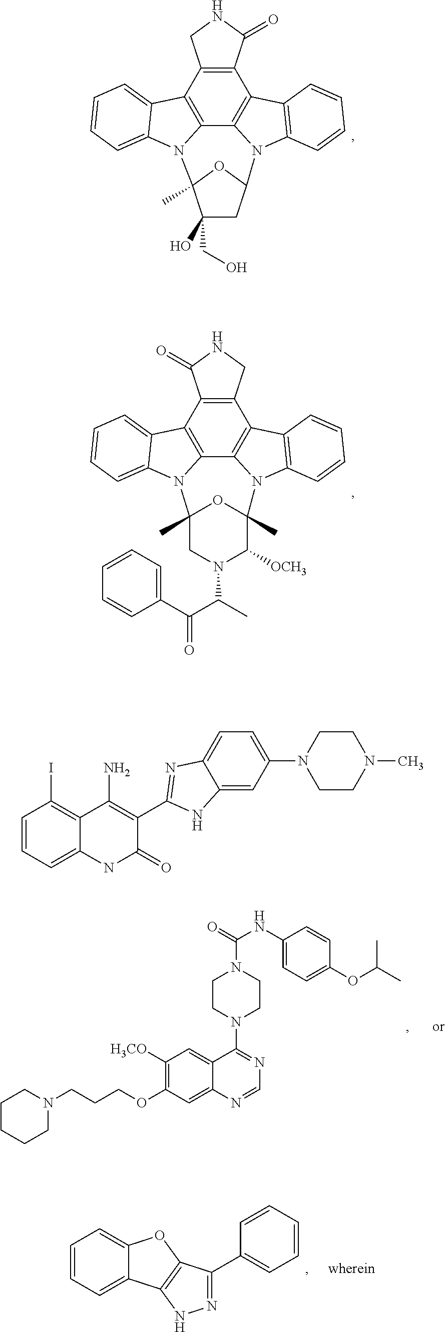

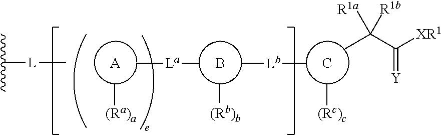

is attached to a modifiable carbon, oxygen, nitrogen or sulfur atom.

In some embodiments, a TBM moiety is selected from

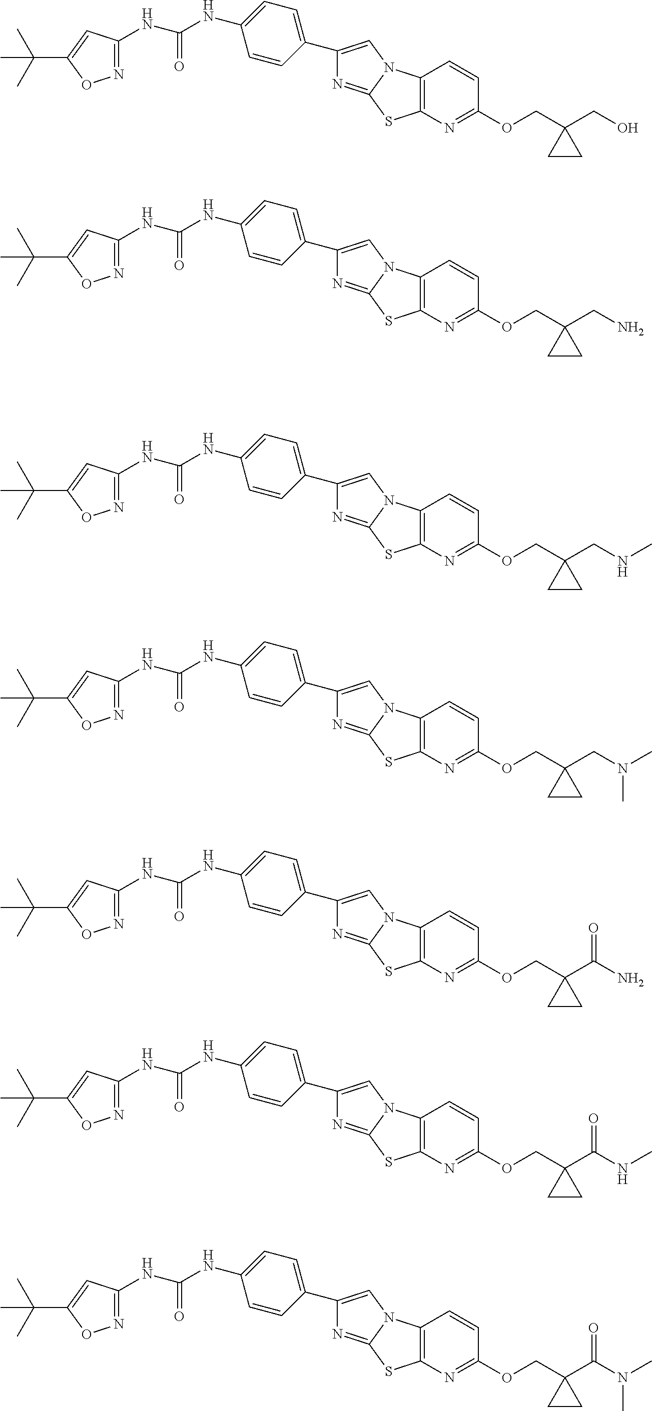

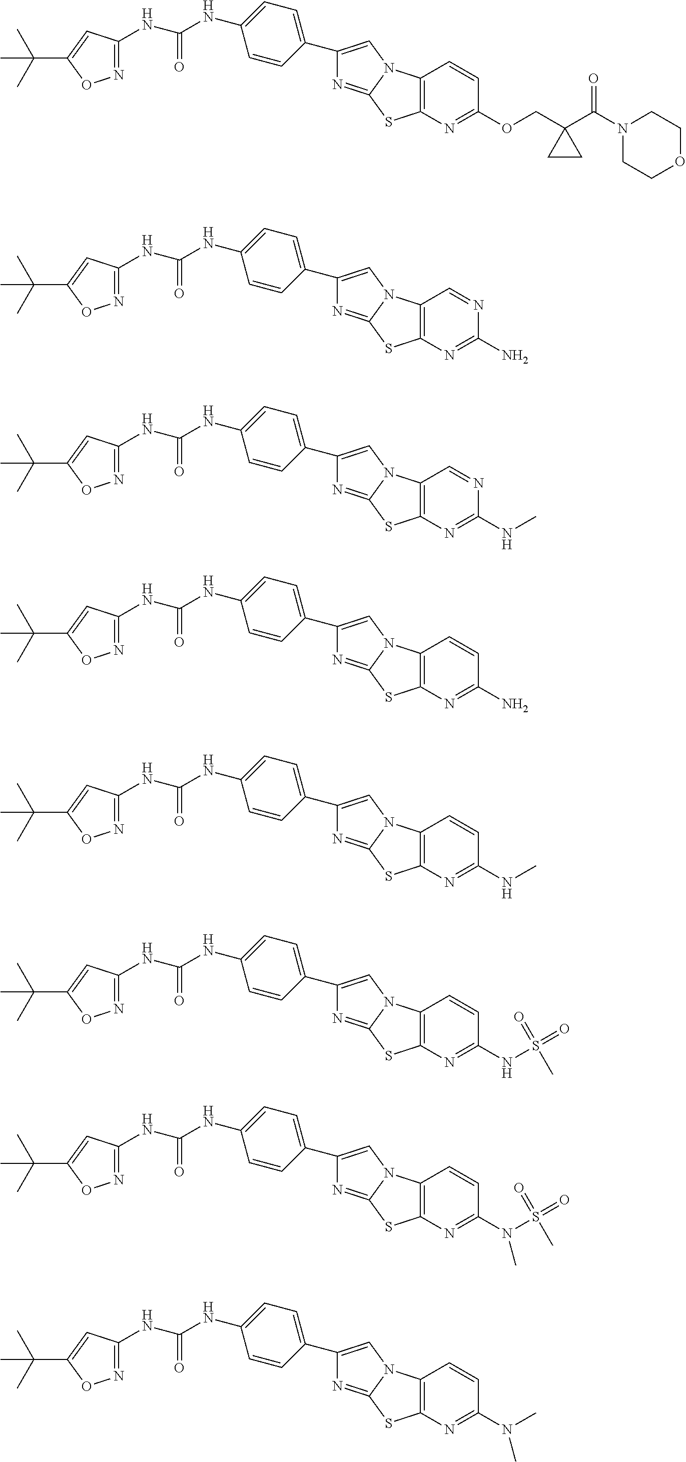

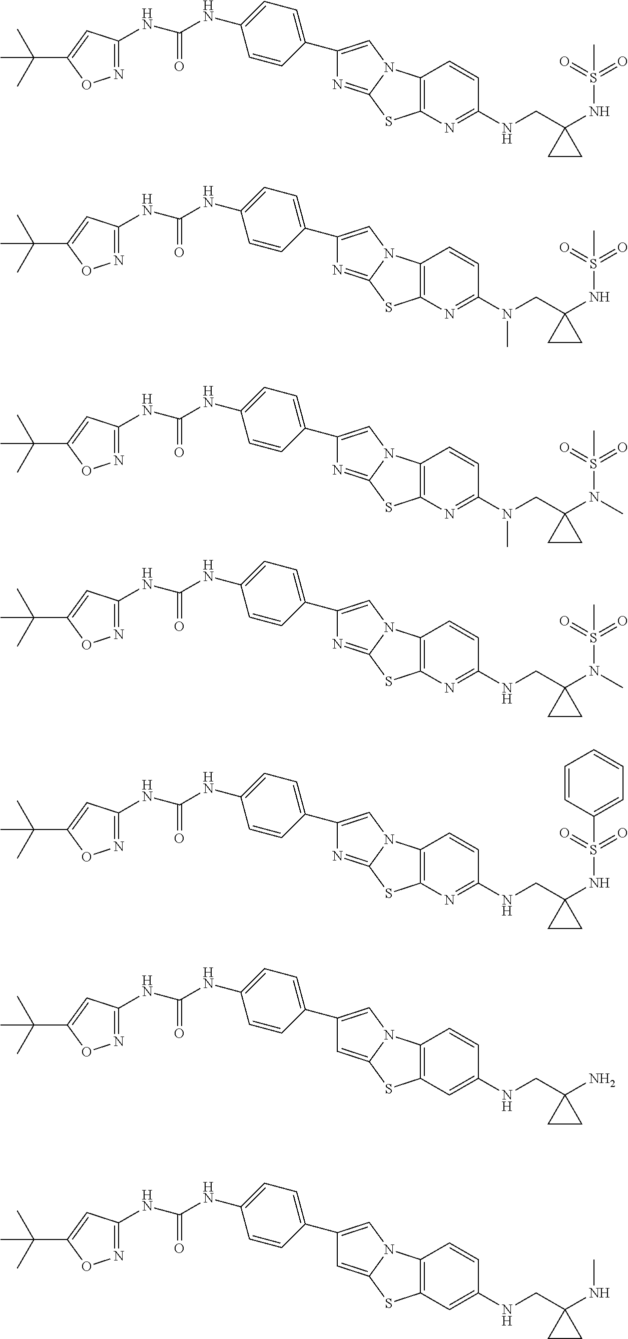

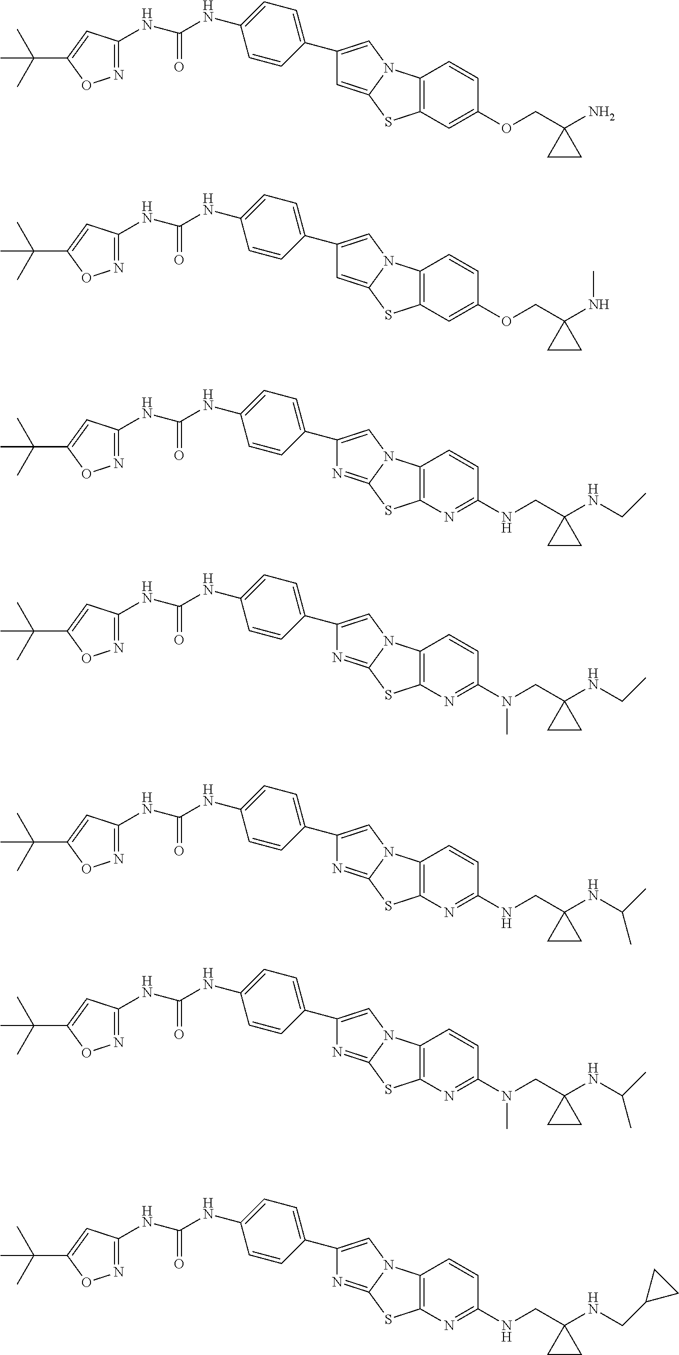

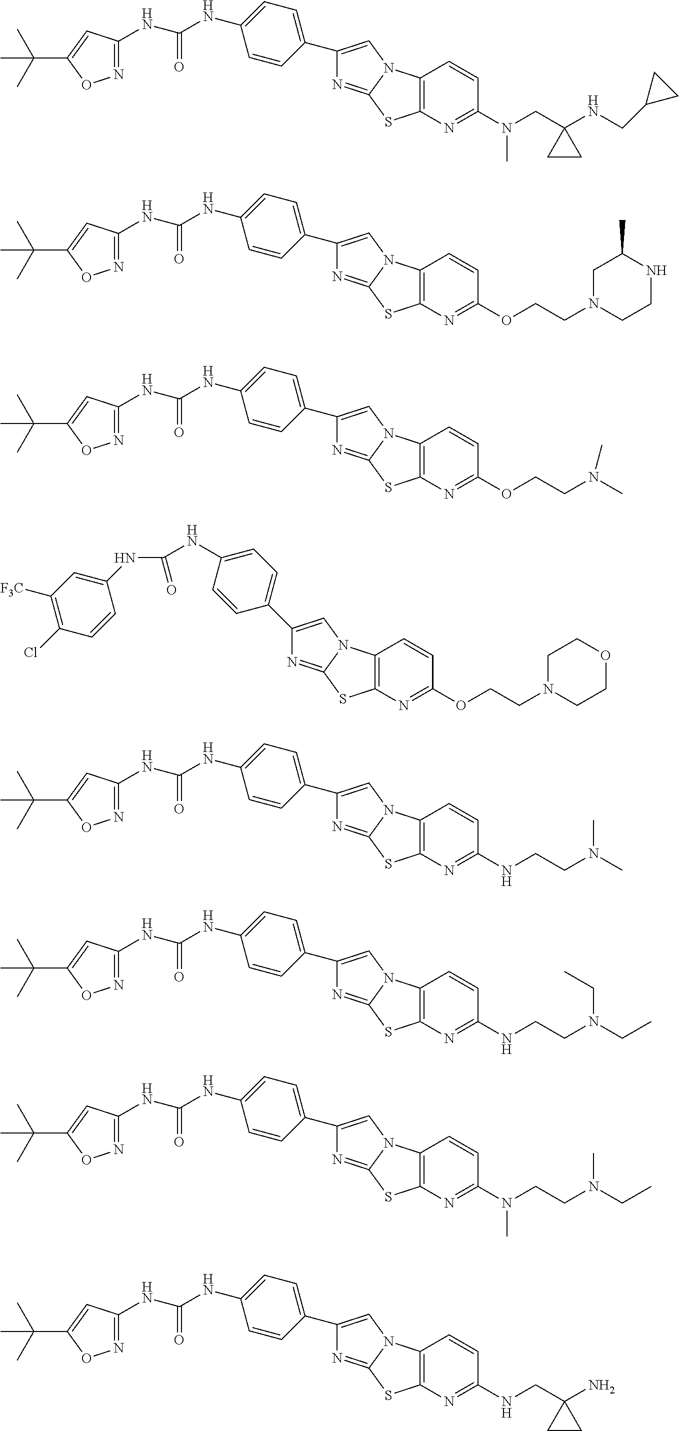

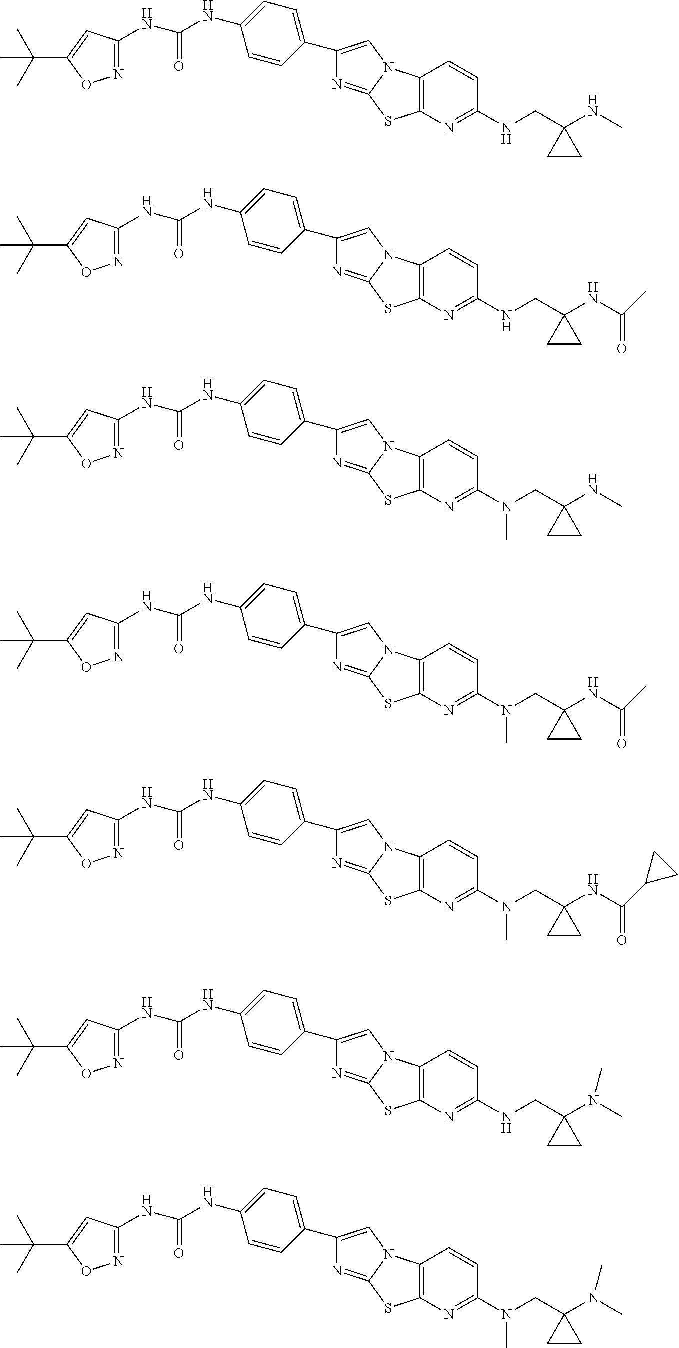

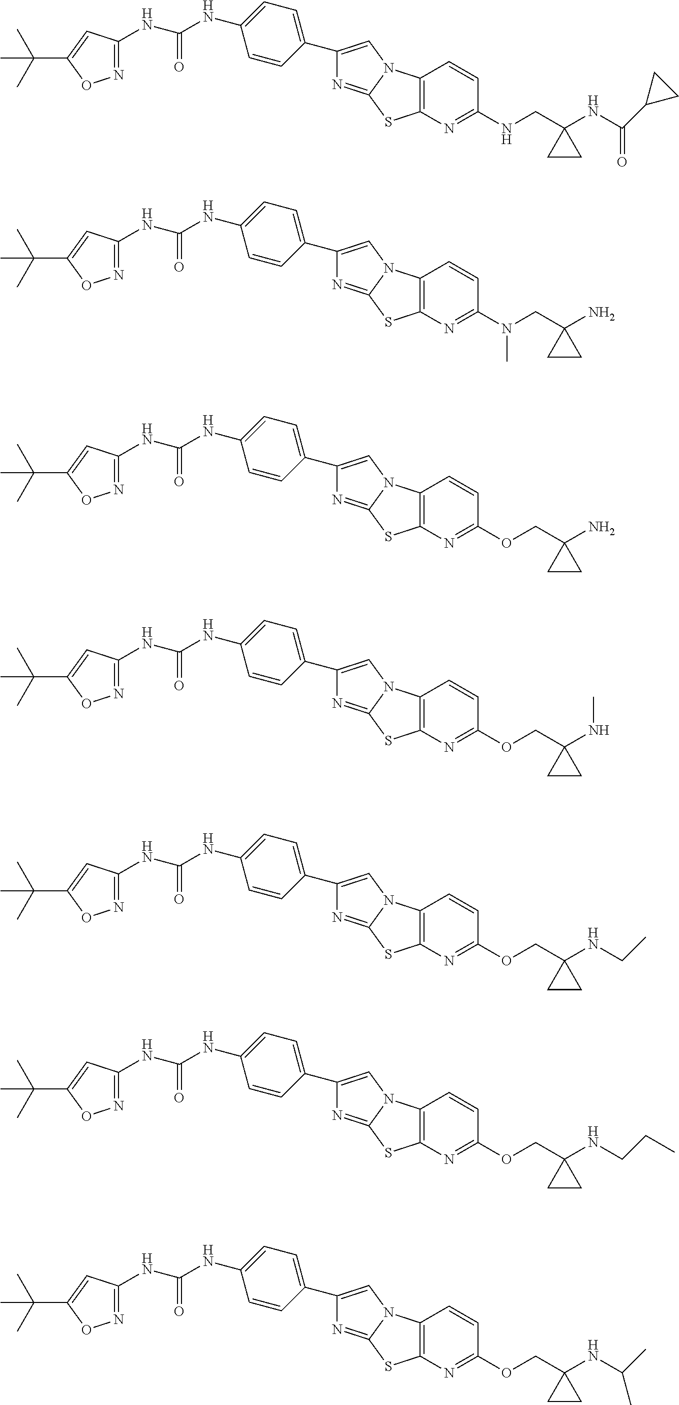

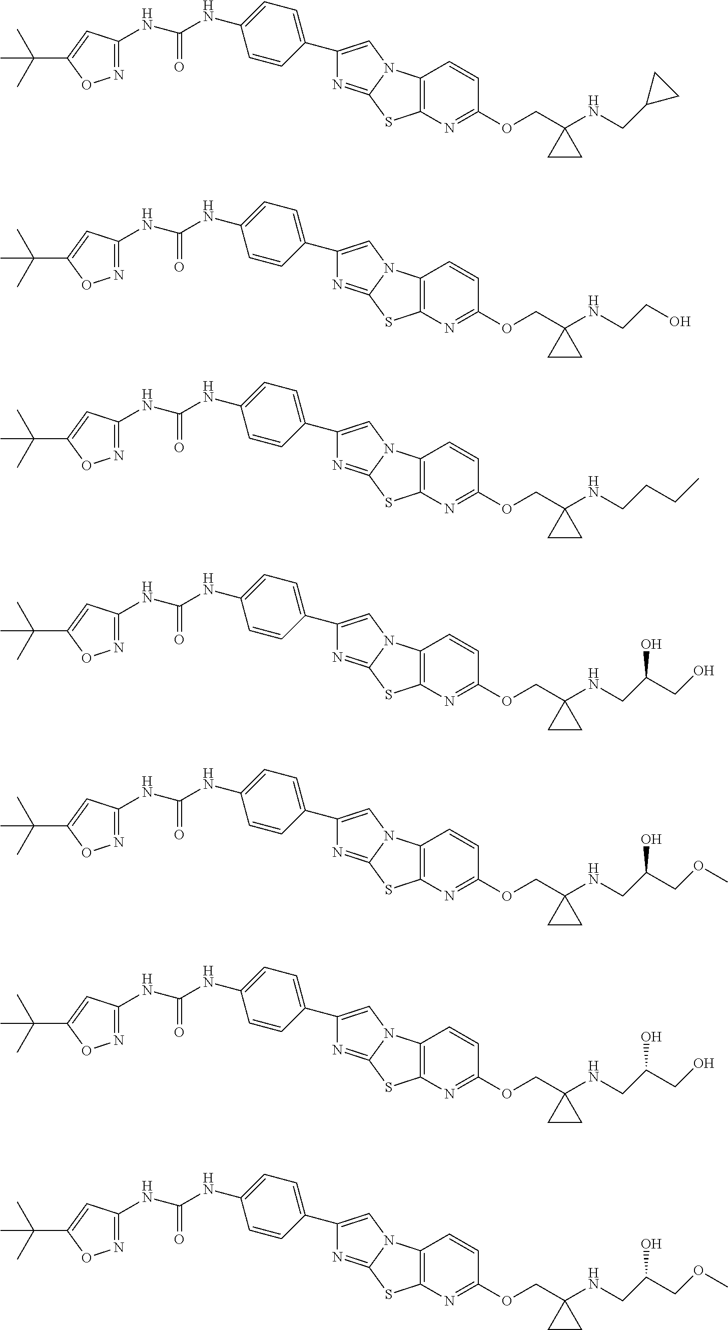

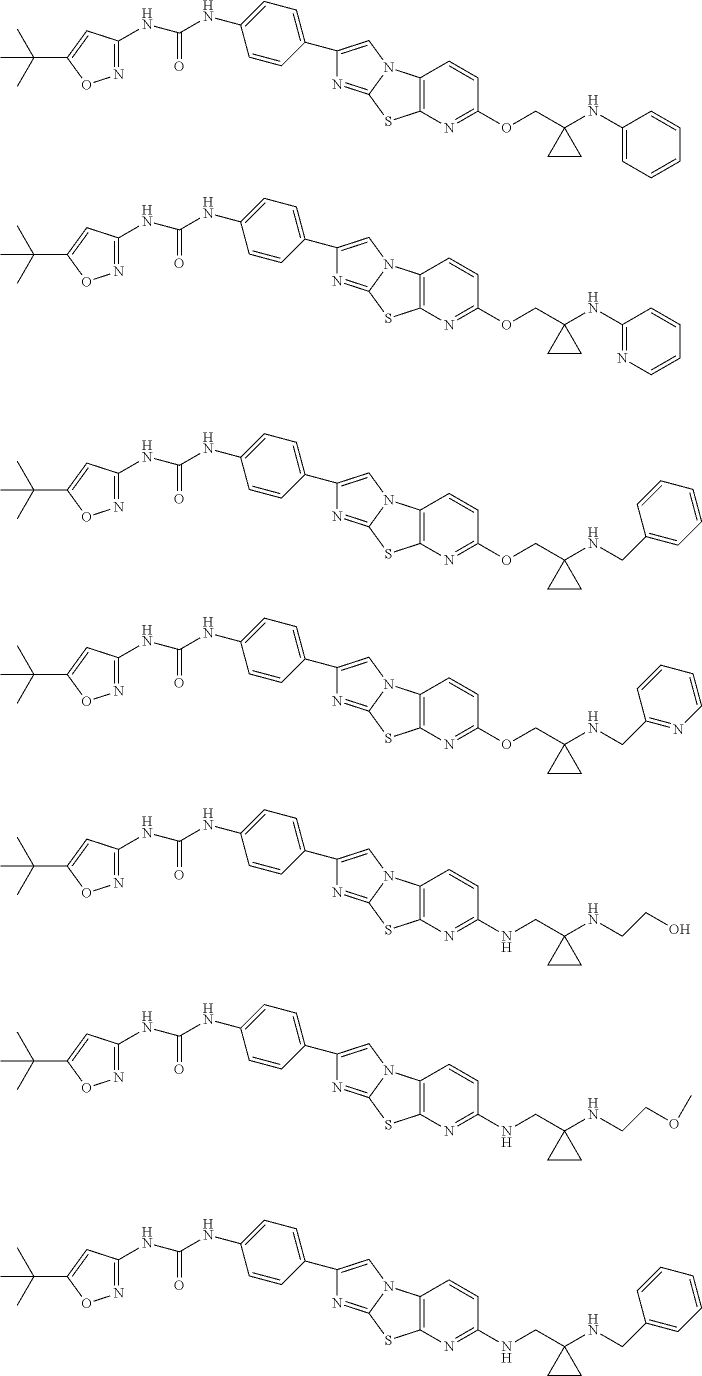

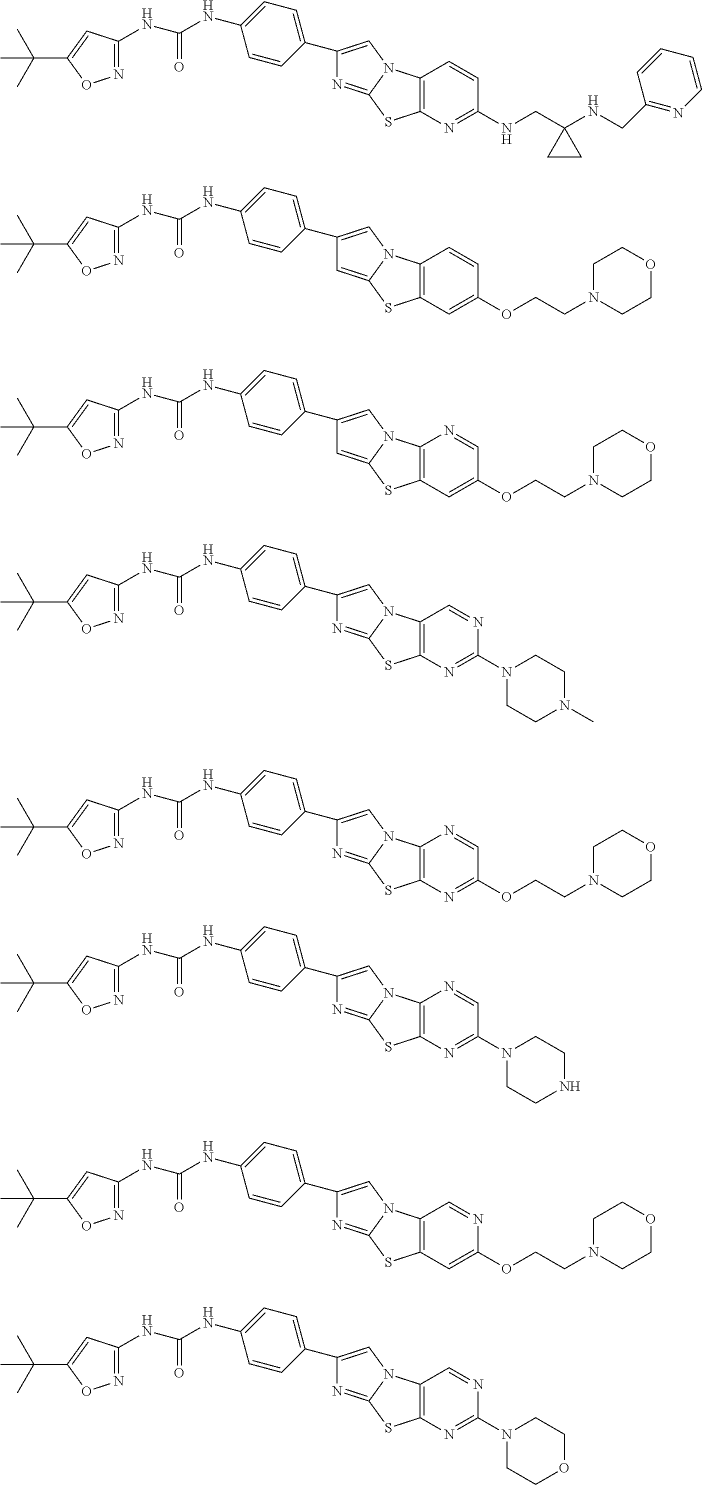

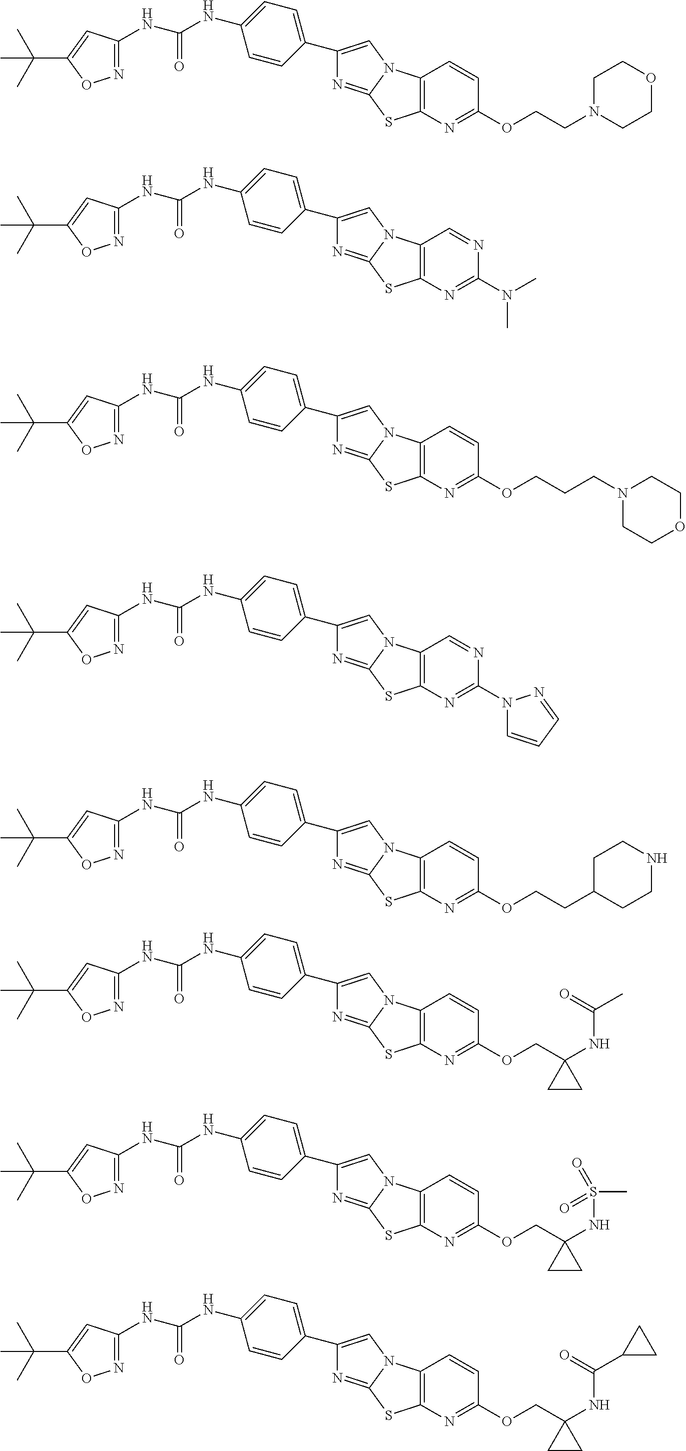

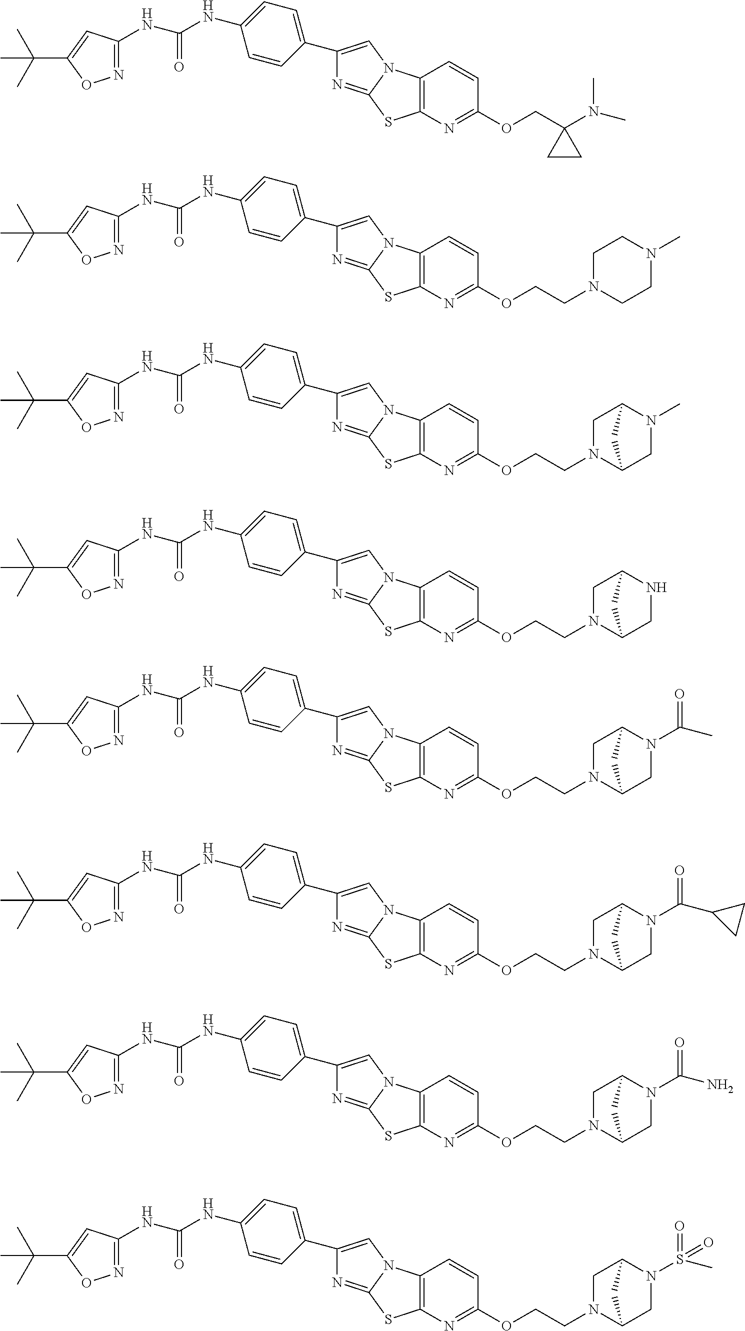

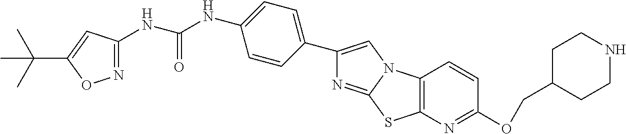 [SMILES: CC(C)(C)c1cc(NC(=O)Nc2ccc(-c3cn4c(n3)sc3nc(OCC5CCNCC5)ccc34)cc2)no1]

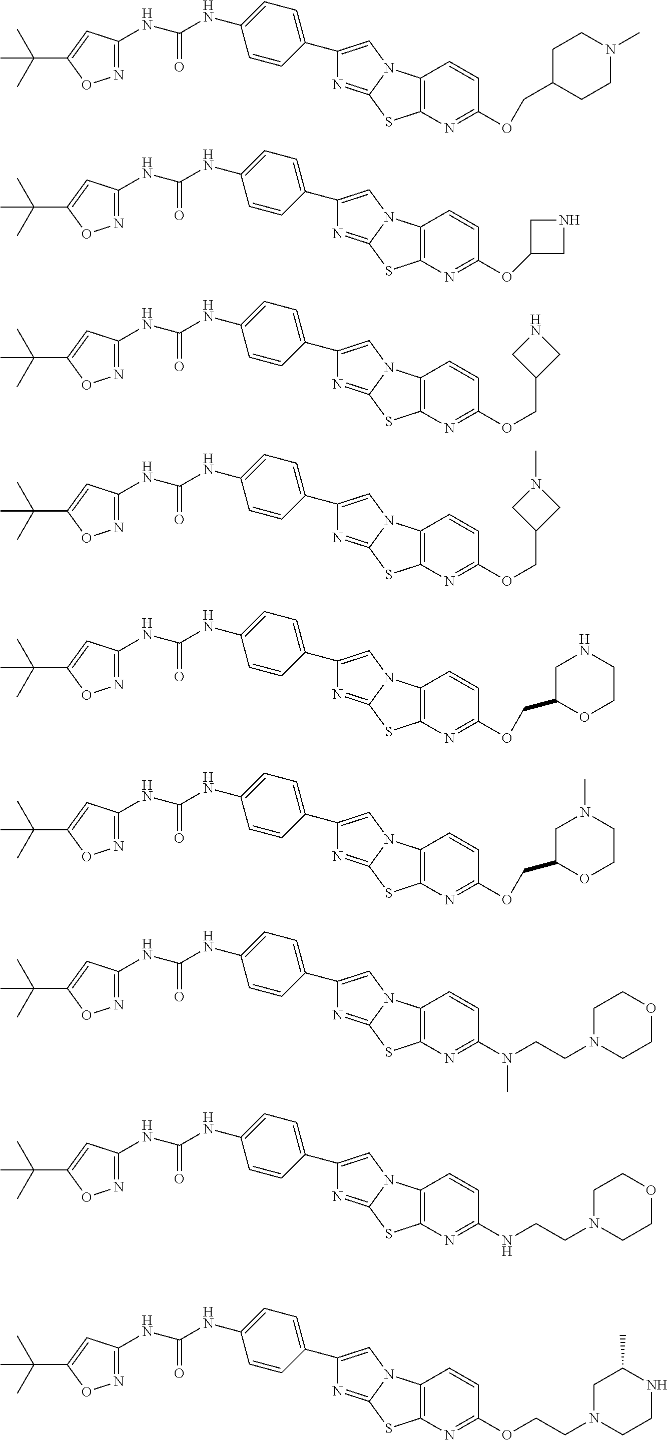

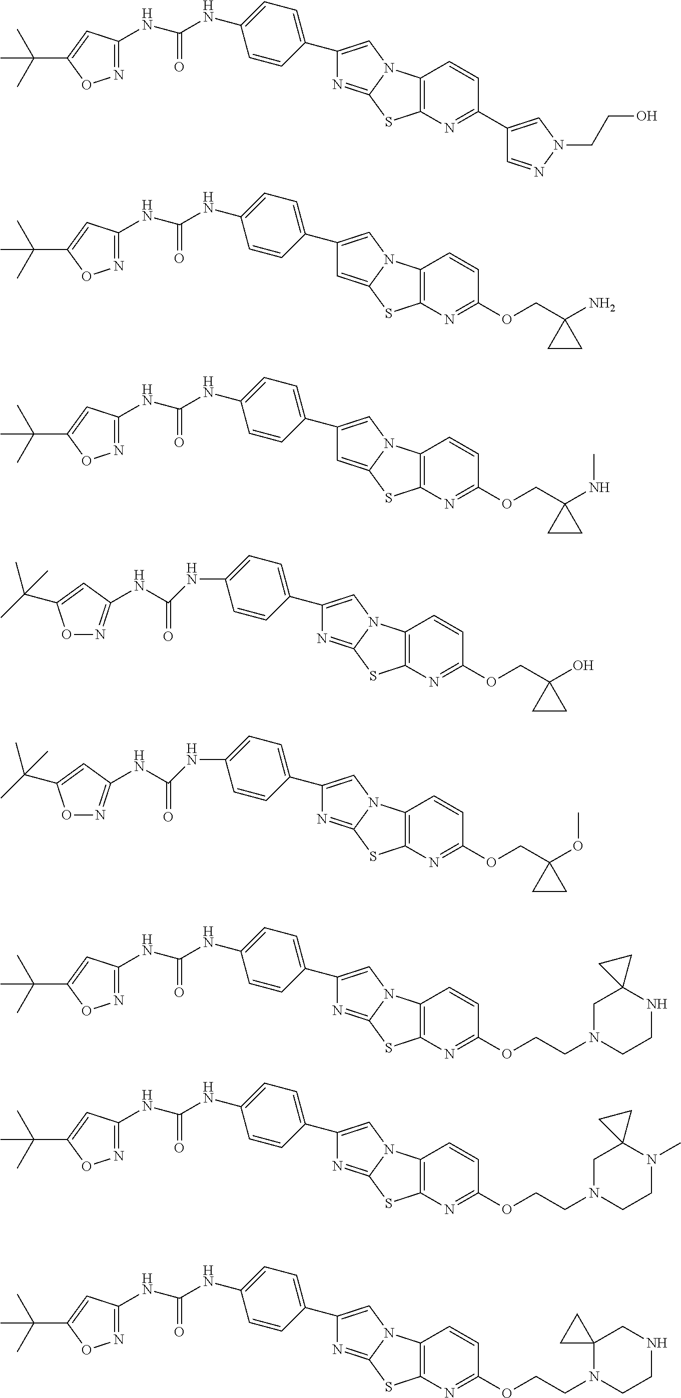

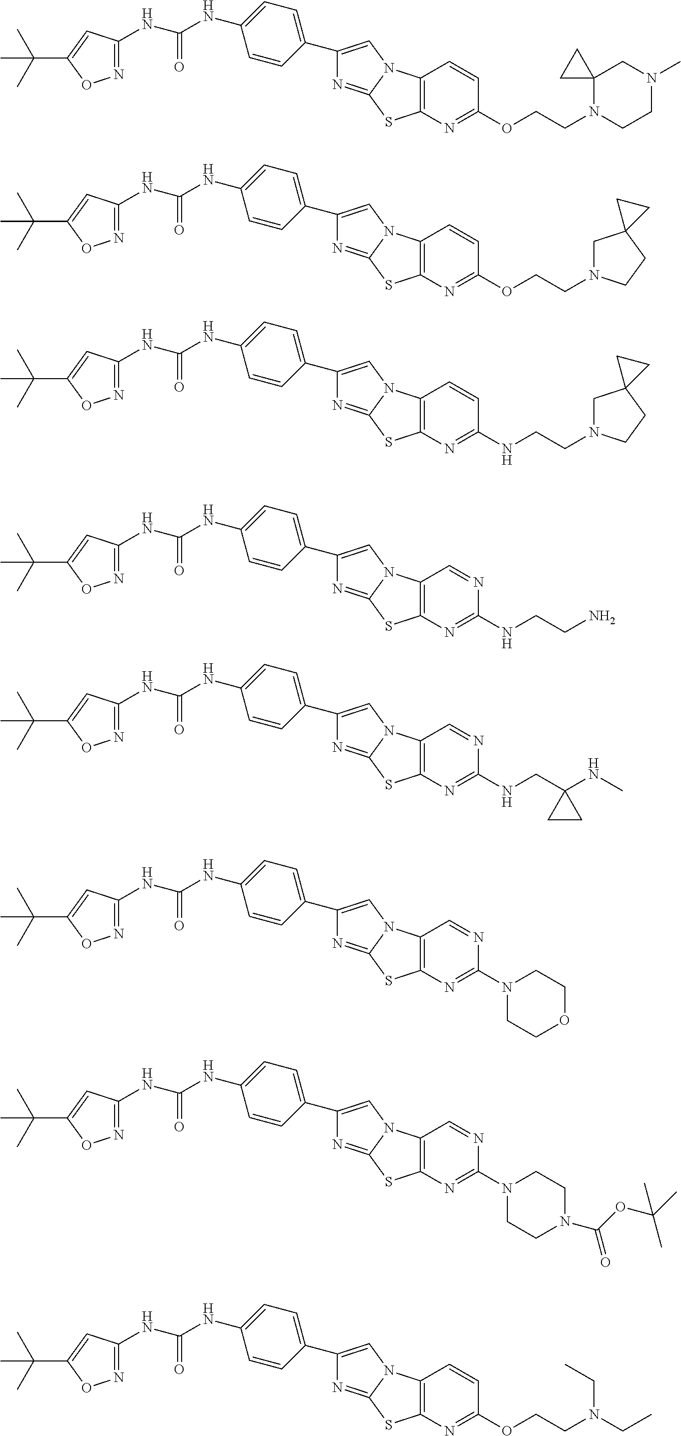

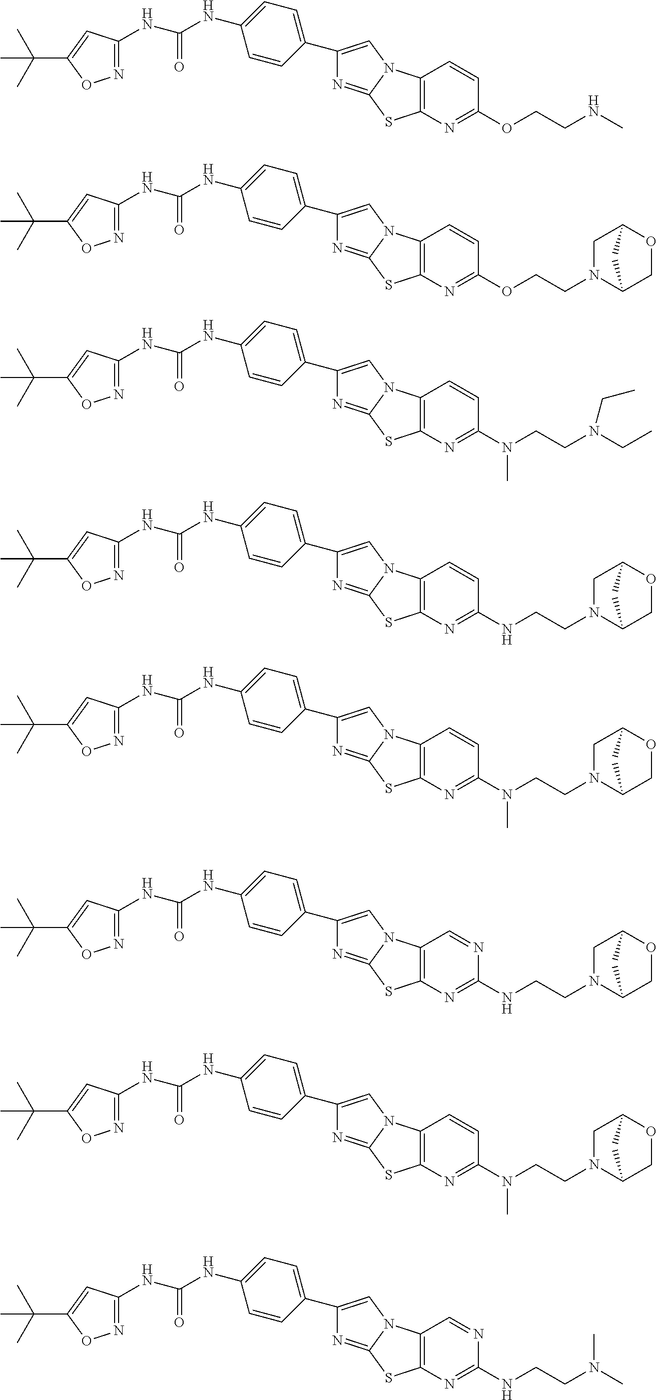

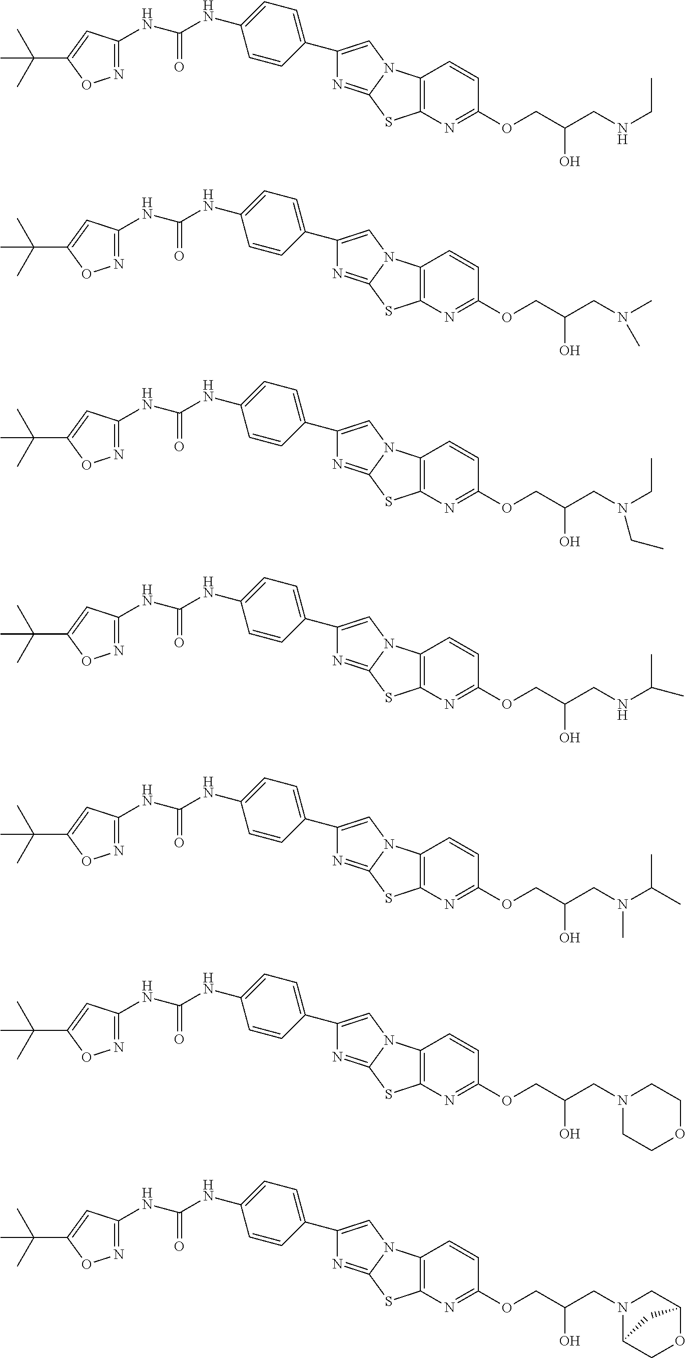

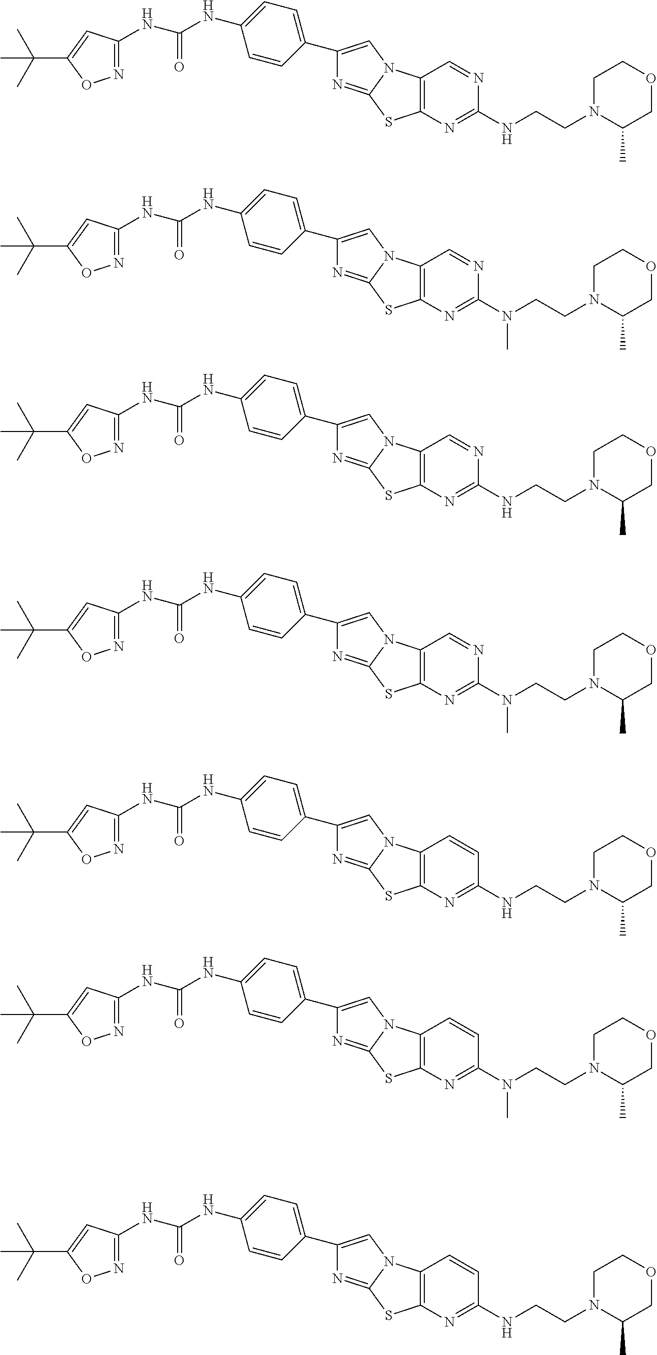

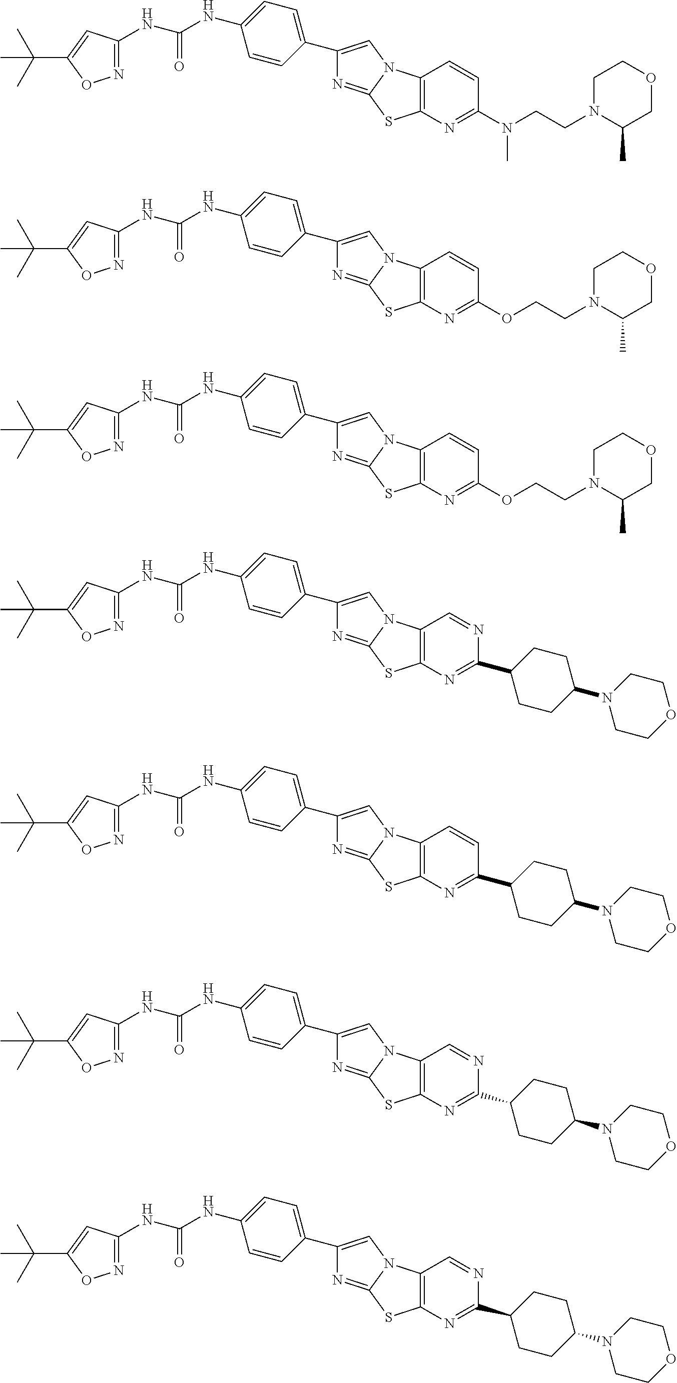

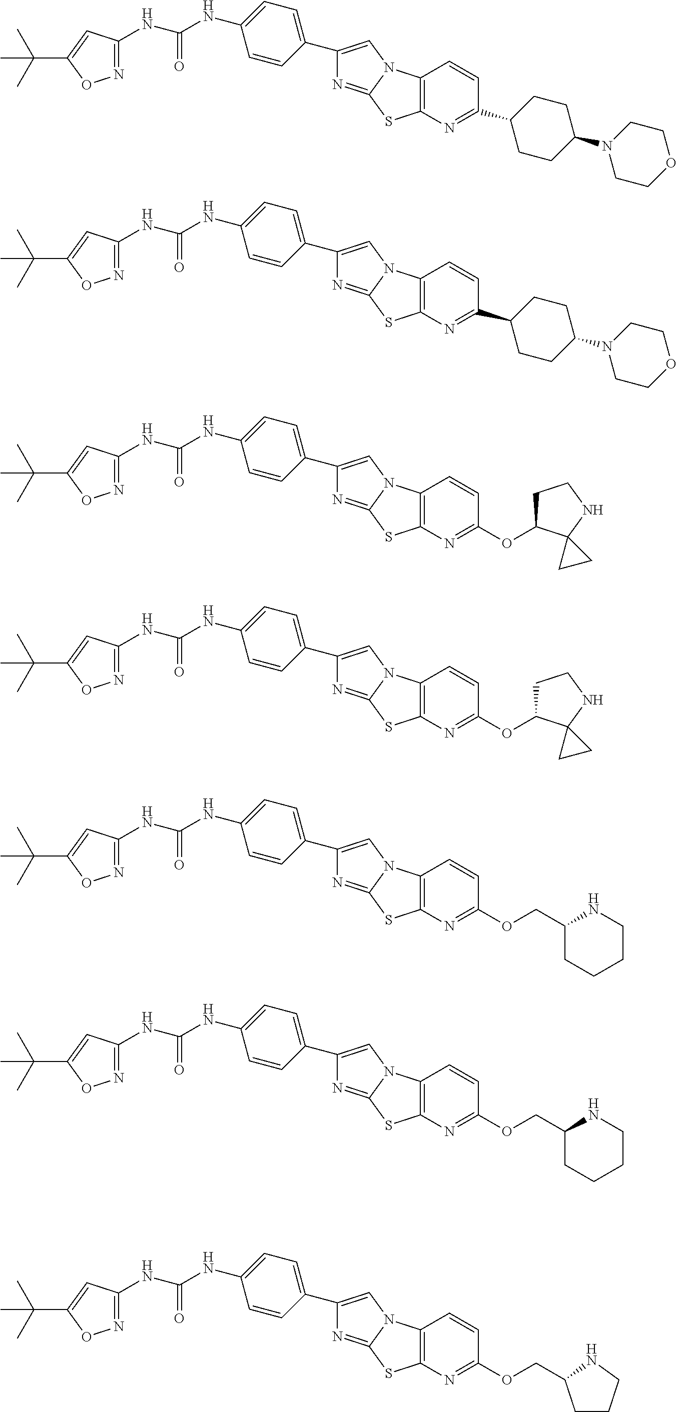

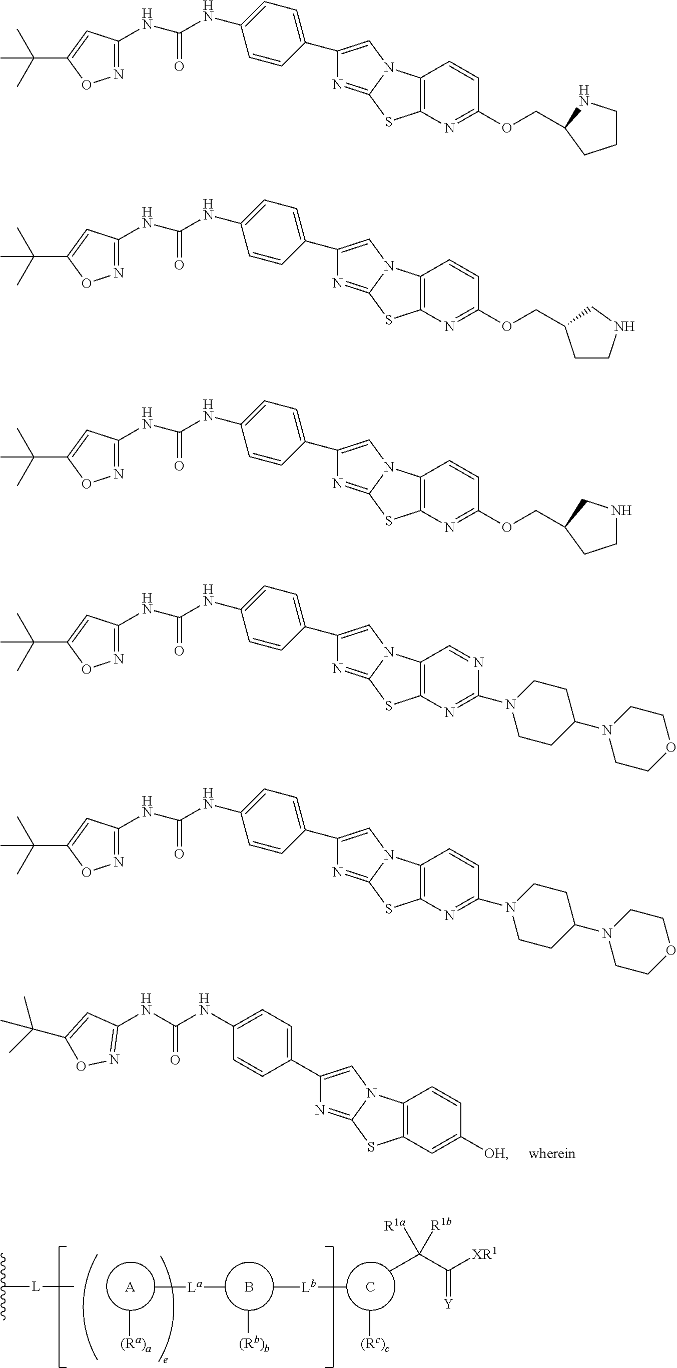

is attached to a modifiable carbon, oxygen, nitrogen or sulfur atom.

In some embodiments, a TBM moiety is selected from

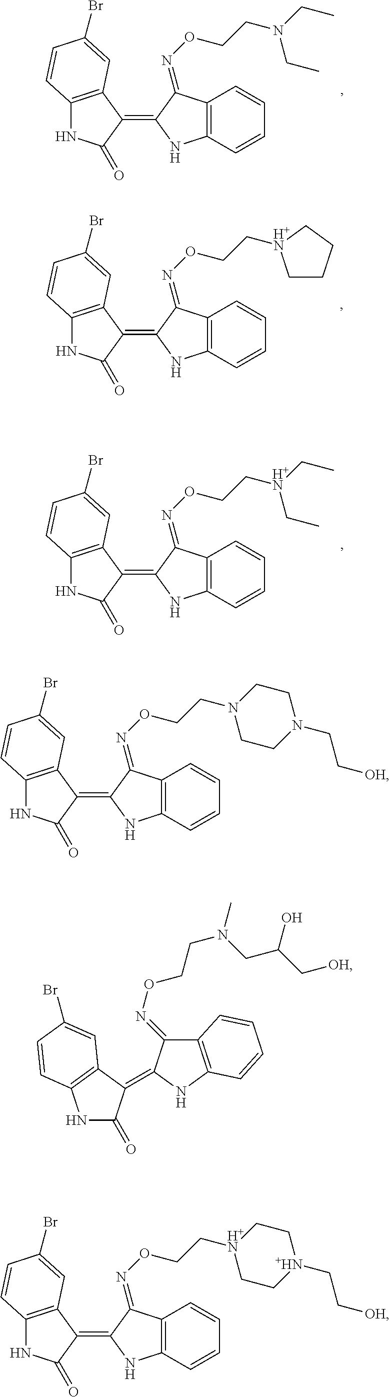

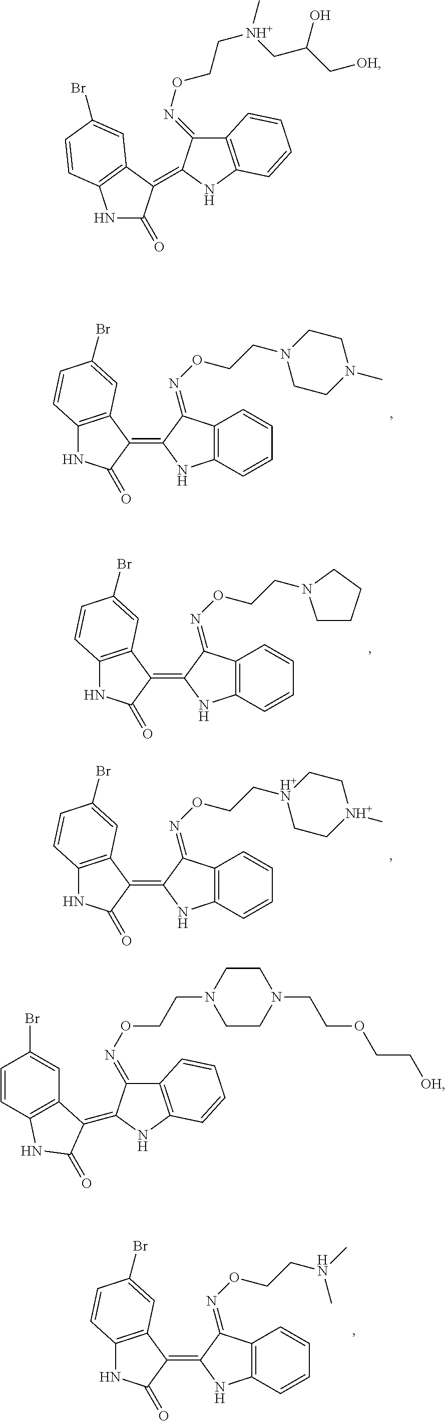

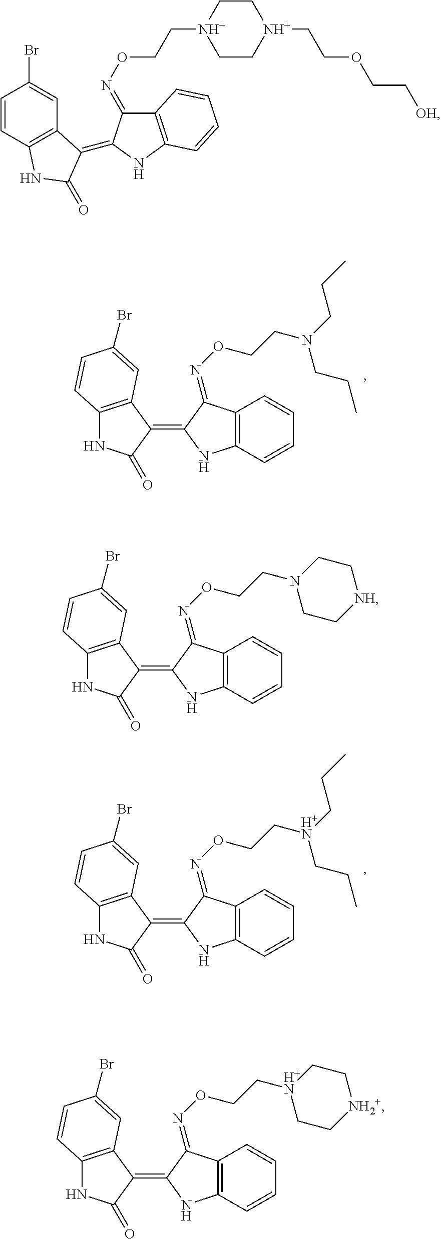

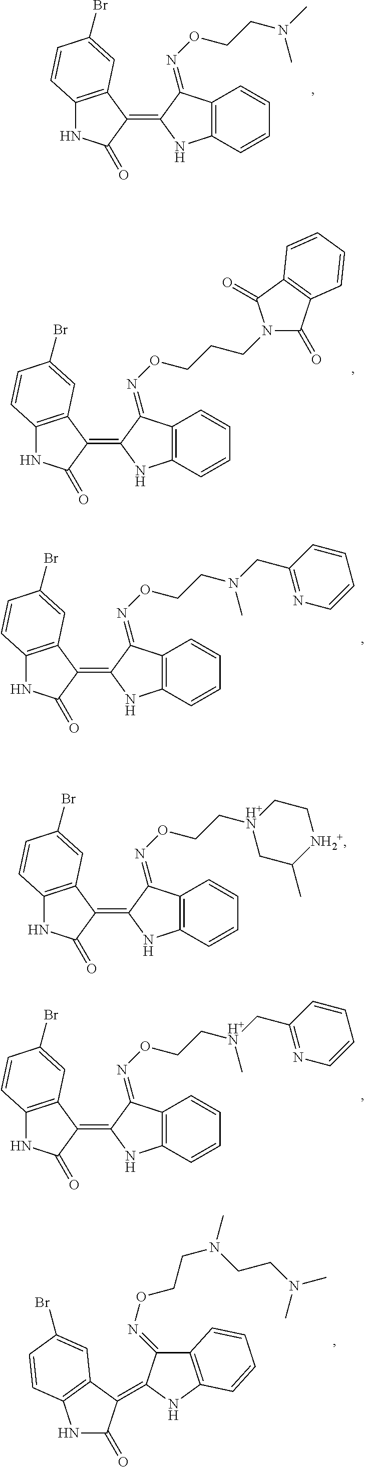

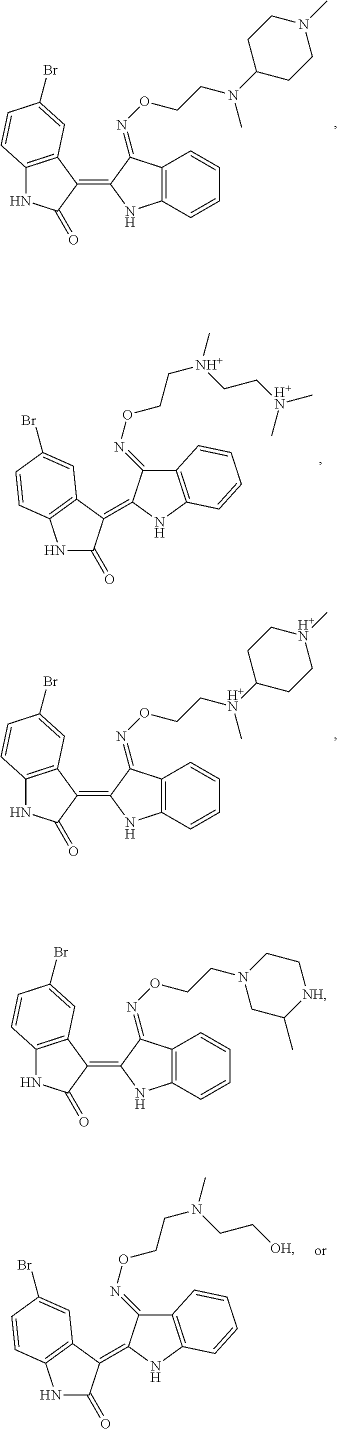

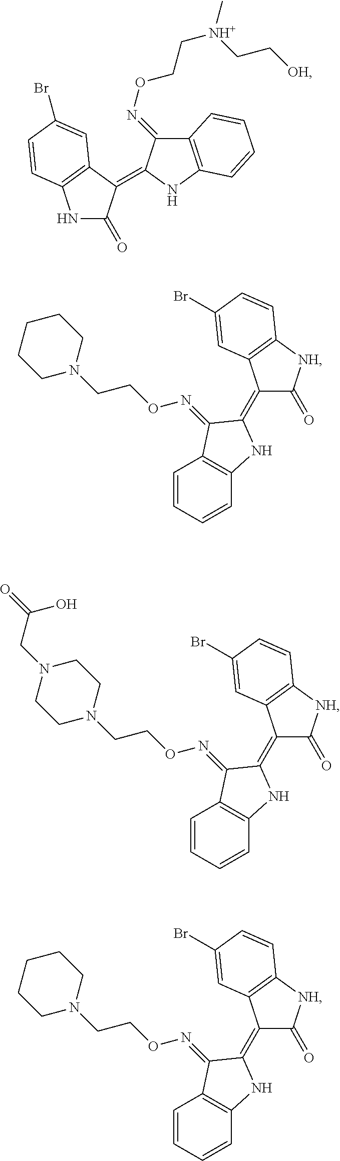 [SMILES: C[NH+](CCO)CCO/N=C1/C(=C2/C(=O)Nc3ccc(Br)cc32)Nc2ccccc21 ; O=C1Nc2ccc(Br)cc2/C1=C1/Nc2ccccc2/C1=N\OCCN1CCCCC1 ; O=C(O)CN1CCN(CCO/N=C2/C(=C3/C(=O)Nc4ccc(Br)cc43)Nc3ccccc32)CC1 ; O=C1Nc2ccc(Br)cc2/C1=C1/Nc2ccccc2/C1=N\OCCN1CCCCC1]

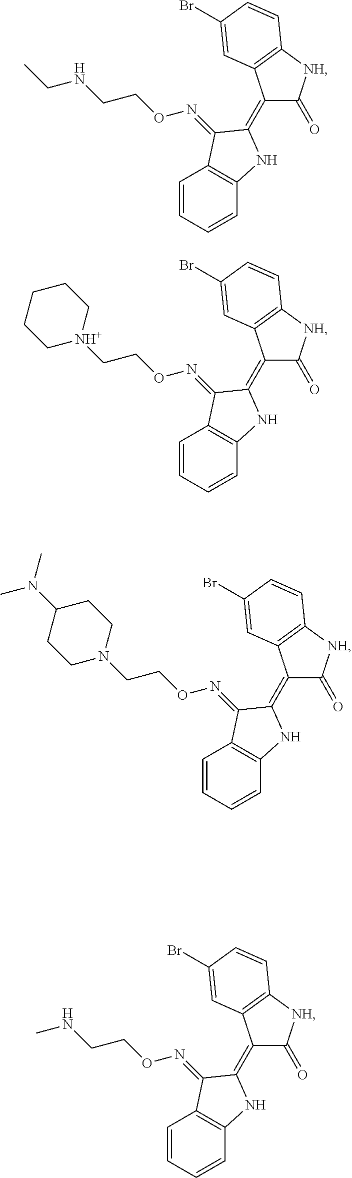 [SMILES: CCNCCO/N=C1/C(=C2/C(=O)Nc3ccc(Br)cc32)Nc2ccccc21 ; O=C1Nc2ccc(Br)cc2/C1=C1/Nc2ccccc2/C1=N\OCC[NH+]1CCCCC1 ; CN(C)C1CCN(CCO/N=C2/C(=C3/C(=O)Nc4ccc(Br)cc43)Nc3ccccc32)CC1 ; CNCCO/N=C1/C(=C2/C(=O)Nc3ccc(Br)cc32)Nc2ccccc21]

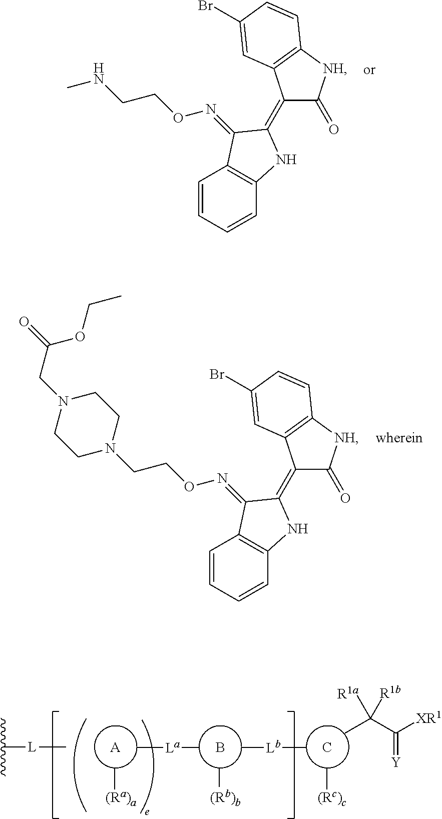

is attached to a modifiable carbon, oxygen, nitrogen or sulfur atom.

In some embodiments, a TBM moiety is selected from

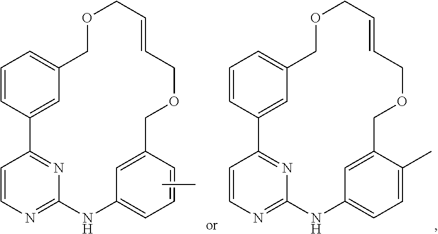

wherein [symbol] denotes attachment to

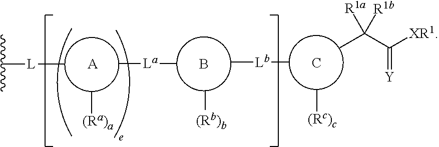

In some embodiments, a TBM moiety is selected from

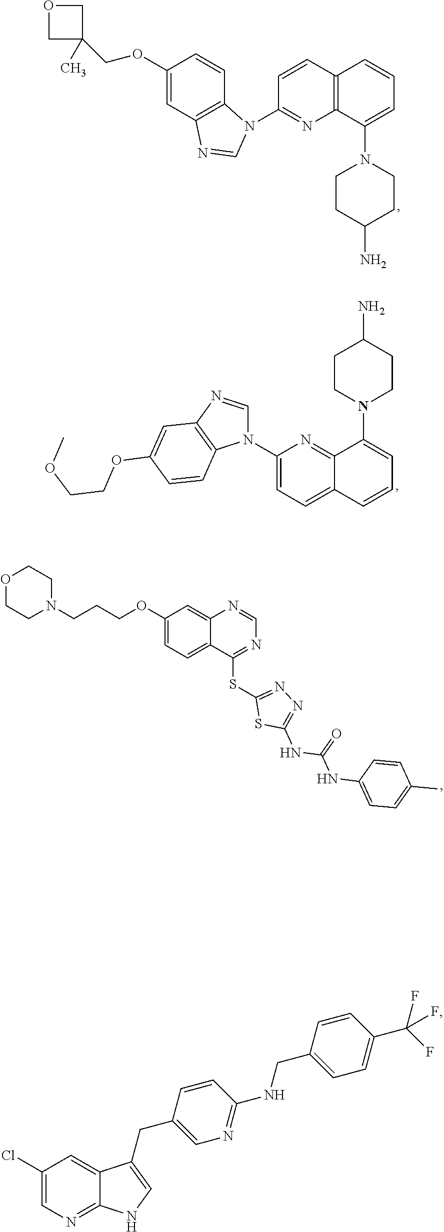

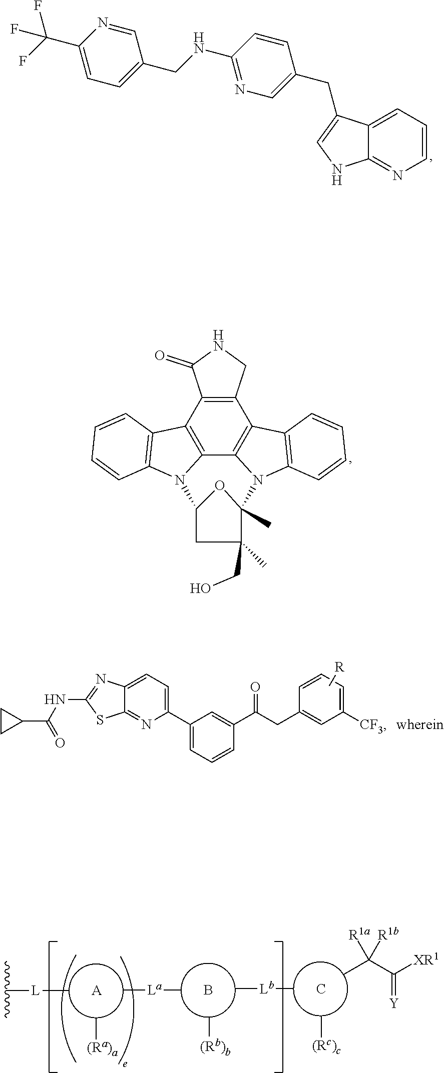

is attached to a modifiable carbon, oxygen, nitrogen or sulfur atom; R is hydrogen, 5-(4-methyl-1H-imidazol-1-yl), or 4-(N-ethylpiperazin-1-yl)methyl).

In some embodiments, a TBM moiety is a RAF ligand selected from

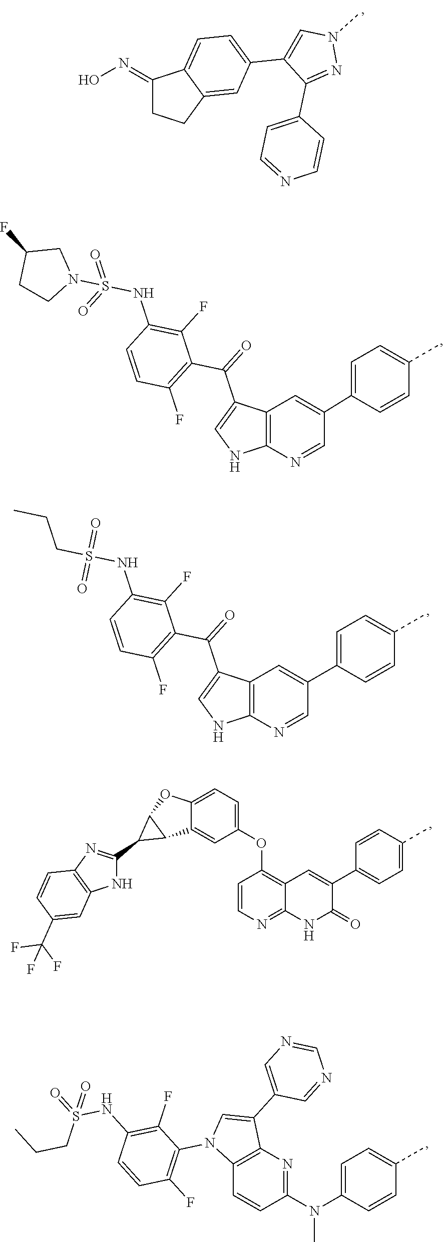

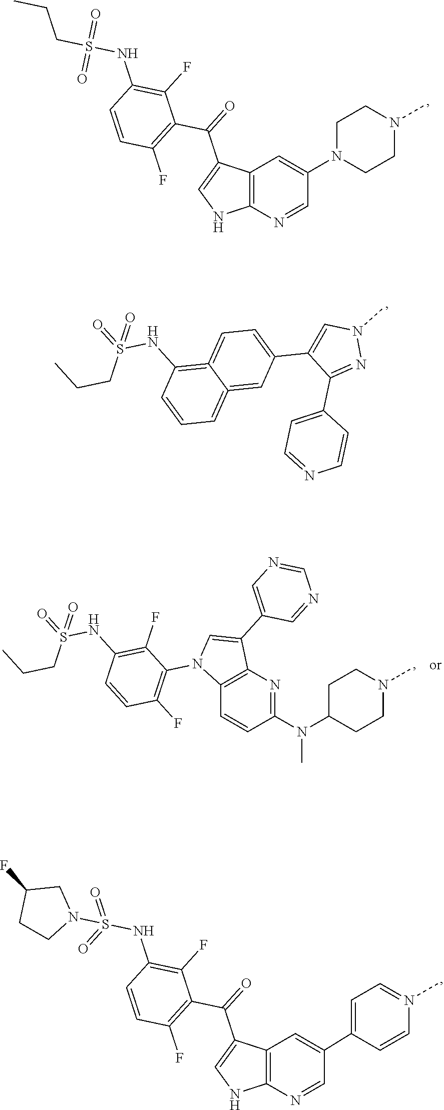

wherein [symbol] denotes attachment to

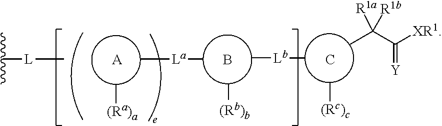

In certain embodiments, the present invention provides a compound of formula I-a, wherein TBM is an receptor tyrosine kinase (RTK) binding moiety

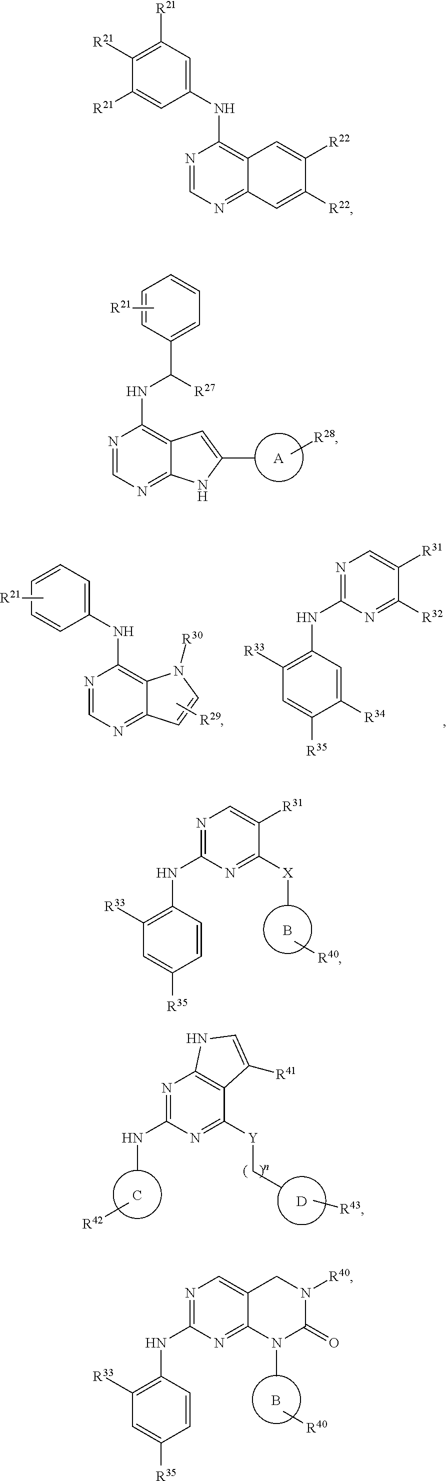

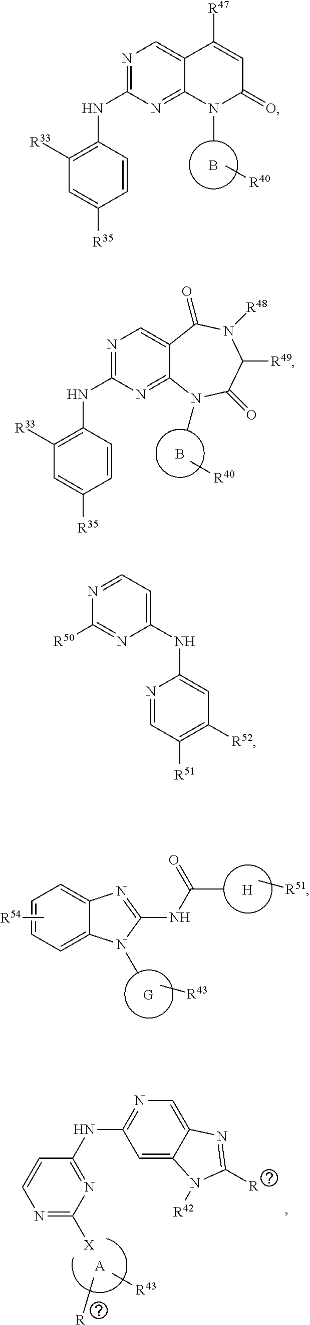

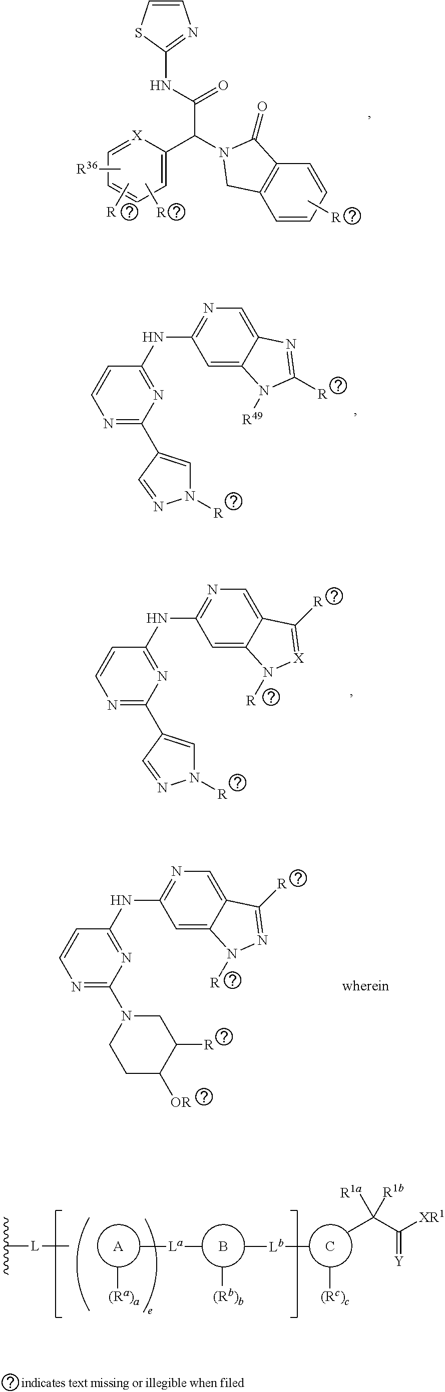

is attached to a modifiable carbon, oxygen, nitrogen or sulfur atom, and wherein L and UBM are as defined above and described in embodiments herein, and wherein each of the variables Ring A, Ring B, Ring C, Ring D, Ring G, Ring H, R²¹, R²², R²⁷, R²⁸, R²⁹, R³⁰, R³¹, R³², R³³, R³⁴, R³⁵, R⁴⁰, R⁴³, R⁴⁴, R⁴⁶, R⁴⁷, R⁴⁸, R⁴⁹, R⁵⁰, R⁵¹, R⁵², R⁵³, R⁵⁴, R^{k1}, R^{k2}, R^{k3}, R^{k4}, R^{k5}, R^{k6}, R^{k7}, R^{k8}, R^{k9}, R^{k10}, R^{k11}, R^{k12}, R^{k13}, R^{k14}, R^{k15}, R^{k16}, R^{k17}, X, Y, and n is as described and defined in WO 2018/118598 and US 2018/0256586, the entirety of each of which is herein incorporated by reference.

In certain embodiments, the present invention provides a compound of formula I-a, wherein TBM is TBK1 binding moiety

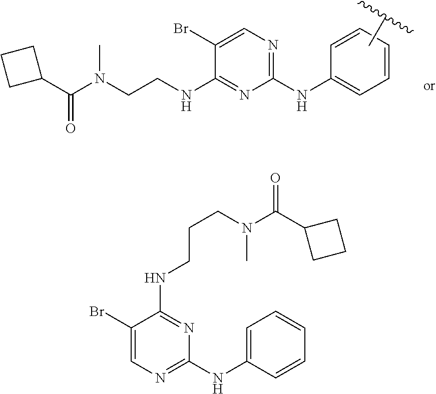

or a pharmaceutically acceptable salt thereof, wherein

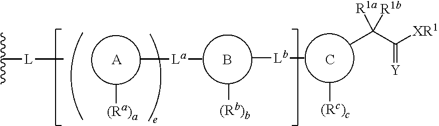

is attached to a modifiable carbon, oxygen, nitrogen or sulfur atom.

In certain embodiments, the present invention provides a compound of formula I-a, wherein TBM is TBK1 binding moiety

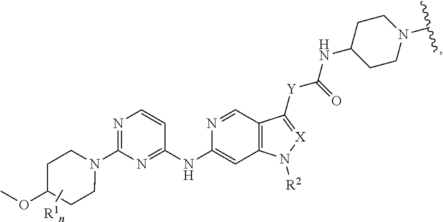

or a pharmaceutically acceptable salt thereof, wherein [symbol] denotes attachment to

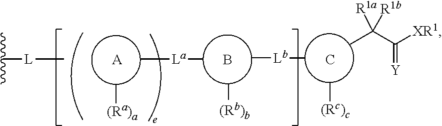

and wherein each of the variables R¹, R², X, Y, and n and as described and defined in WO 2019/121562, the entirety of each of which is herein incorporated by reference.

In certain embodiments, the present invention provides a compound of formula I-a, wherein TBM is a

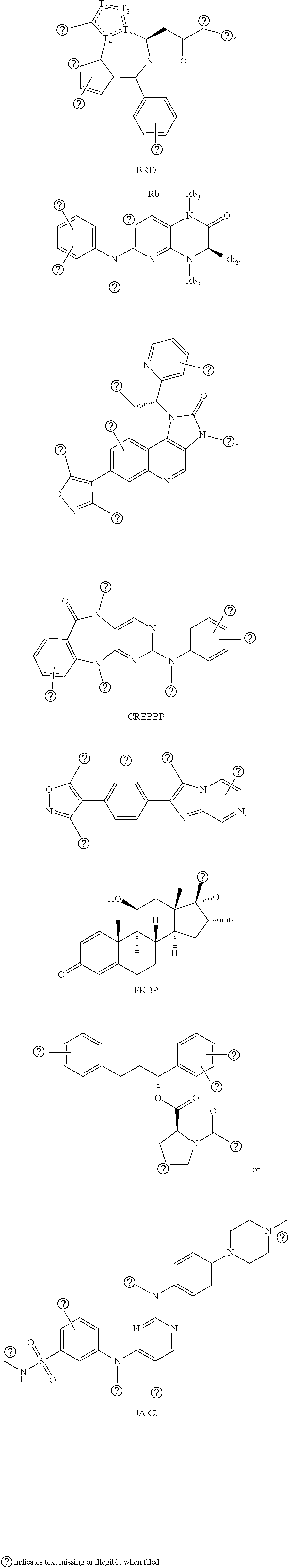

binding moiety or a pharmaceutically acceptable salt thereof, wherein

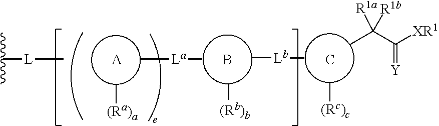

is attached to a modifiable carbon, oxygen, nitrogen or sulfur atom, and wherein each of the variables T1-T7, A1, A2, Raj-Ra4, Rb1-Rb7, Rc1-Rc5, Rd1-Rd3, Re1-Re3, Rf1, Rg1-Rg3, Rh1-Rh5, and nn1-nn12 as described and defined in U.S. Pat. Nos. 9,694,084 and 10,125,114, the entirety of each of which is herein incorporated by reference.

In certain embodiments, the present invention provides a compound of formula I-a, wherein TBM is TBK1 binding moiety

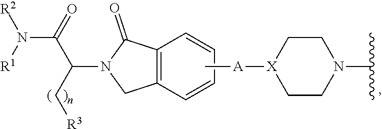

or a pharmaceutically acceptable salt thereof, wherein [symbol] denotes attachment to

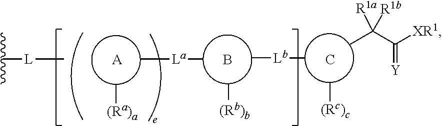

and wherein each of the variables R¹, R², R³, X, A, and n and as described and defined in WO 2017/1855036 and US 2019/0106417, the entirety of each of which is herein incorporated by reference.

In certain embodiments, the present invention provides a compound of formula I-a, wherein TBM is an androgen receptor binding moiety

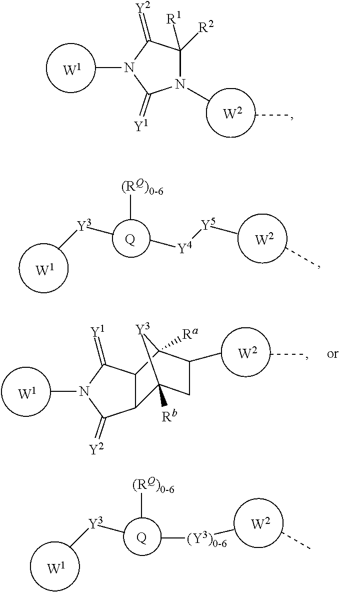

or a pharmaceutically acceptable salt thereof, wherein [symbol] denotes attachment to

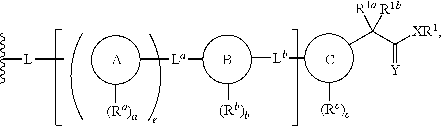

and wherein each of the variables W1, W2, Y₁, Y₂, Y₃, YA, Y₅, R^{a}, R^{b}, and RR is as described and defined in WO 2016/118666, US 2016/0214972, US 2017/327469, and WO 2019/023553, the entirety of each of which is herein incorporated by reference.

In certain embodiments, the present invention provides a compound of formula I-a, wherein TBM is an androgen receptor binding moiety

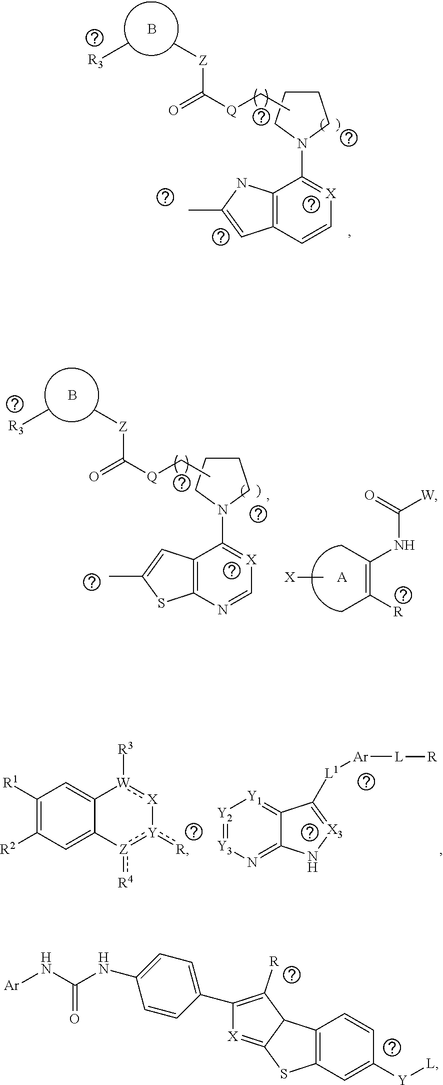

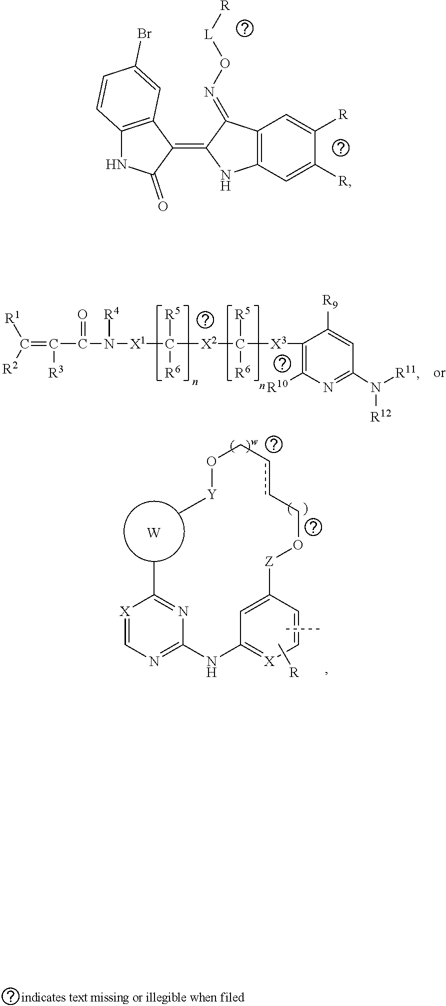

or a pharmaceutically acceptable salt thereof, wherein [symbol] denotes attachment to

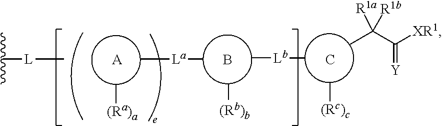

and wherein each of the variables Ring A, Ring B, Ring W, Ar, Ar₁, L, L¹, L², Q, R, R¹, R², R³, R⁴, R⁵, R⁶, R⁷, R⁸, R⁹, R¹⁰, R¹¹, R¹², R¹, R³, R⁵, R⁶, W, X, X¹, X², X³, X₁, X₂, X₃, X₄, Y, Y₁, Y₂, Y₃, Z, Z¹, m, n, q, and z is as described and defined in WO 2018/118598 and US 2018/0256586, the entirety of each of which is herein incorporated by reference.

In certain embodiments, the present invention provides a compound of formula I-a, wherein TBM is a HER binding moiety

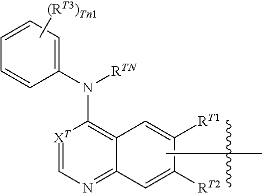

or a pharmaceutically acceptable salt thereof, wherein [symbol] denotes attachment to

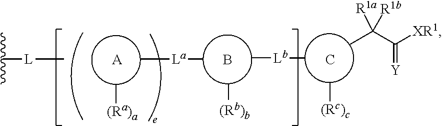

and wherein each of the variables RIN, R^{T1}, R^{T2}, R^{T3}, XT, and Tnl is as described and defined in WO 2017/117474 and US 2019/0016703, the entirety of each of which is herein incorporated by reference.

In certain embodiments, the present invention provides a compound of formula I-a, wherein TBM is a TAU binding moiety

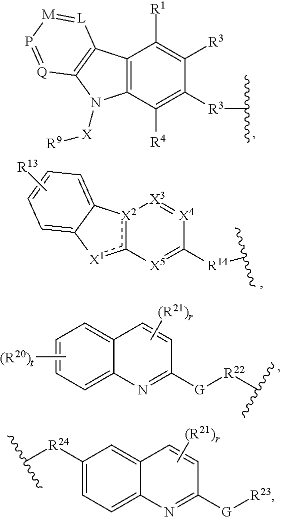

or a pharmaceutically acceptable salt thereof, wherein [symbol] denotes attachment to

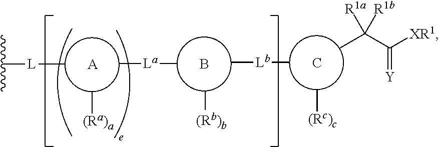

and wherein each of the variables R¹, R², R³, R⁴, R⁹, R¹³, R¹⁴, R²⁰, R²¹, R²², R²³, R²⁴, X¹, X², X³, X⁴, X³, G, L, M, P, Q, t, and r is as described and defined in WO 2019/014429, the entirety of each of which is herein incorporated by reference. In certain embodiments, the present invention provides a compound of formula I or II, wherein TBM is an estrogen receptor binding moiety

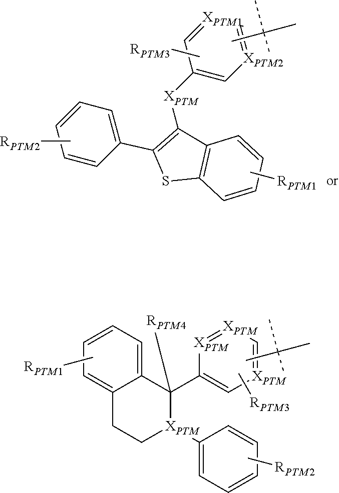

or a pharmaceutically acceptable salt thereof, wherein [symbol] denotes attachment to

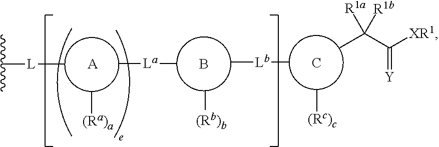

and wherein each of the variables R_{PTM1}, R_{PTM2}, R_{PTM3}, R_{PTM4}, X_{PTM}, X_{PTM1}, and X_{PTM2} is as described and defined in WO 2018/144649 and US 2018/0215731, the entirety of each of which is herein incorporated by reference.

In certain embodiments, the present invention provides a compound of formula I-a, wherein TBM is a protein kinase binding moiety

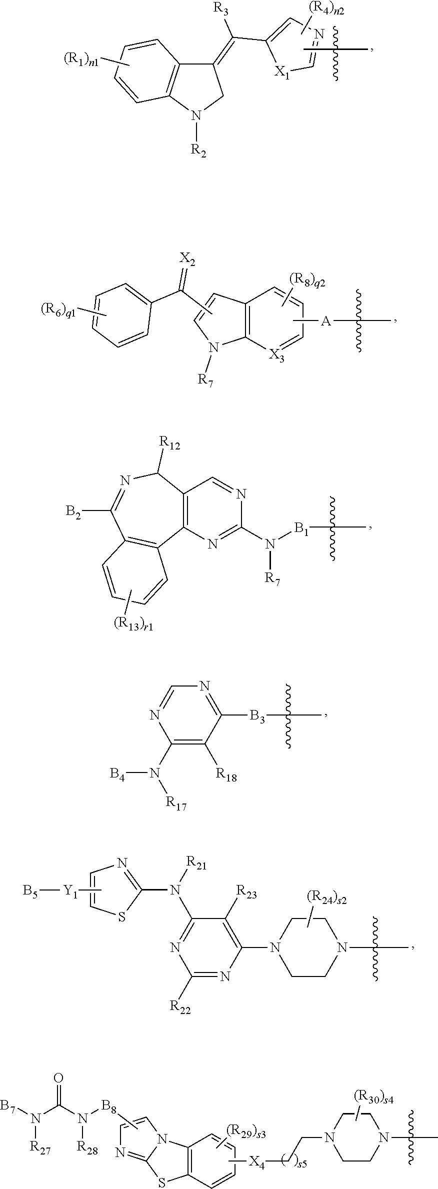

or a pharmaceutically acceptable salt thereof, wherein [symbol] denotes attachment to

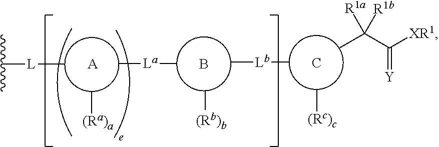

and wherein each of the variables R₁₋₄, R₆₋₈, R₁₂₋₁₄, R₁₇₋₁₈, R₂₁₋₂₄, R₂₇₋₃₀, X₁₋₄, B₂₋₇, Y₁, n1, n2, q1, q2, r1, and s2-5 is as described and defined in WO 2018/098280, the entirety of each of which is herein incorporated by reference.

In certain embodiments, the present invention provides a compound of formula I-a, wherein TBM is a BTK binding moiety

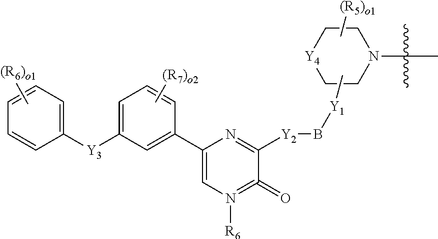

or a pharmaceutically acceptable salt thereof, wherein [symbol] denotes attachment to

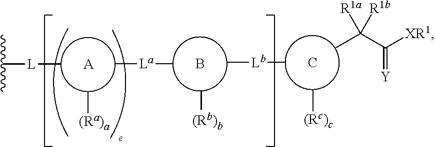

and wherein each of the variables Rₐ, R₅₋₇, B, Y₁₋₄, and o1-3 is as described and defined in WO 2018/098275, the entirety of each of which is herein incorporated by reference.

In certain embodiments, the present invention provides a compound of formula I-a, wherein TBM is a BET/BRD4 binding moiety

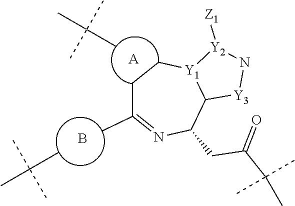

or a pharmaceutically acceptable salt thereof, wherein

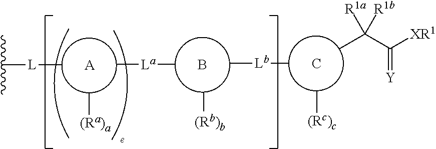

is attached to one [symbol] and wherein each of the variables Ring A, Ring B, Y₁₋₃, and Z₁ is as described and defined in WO 2017/030814 and US 2017/0065719, the entirety of each of which is herein incorporated by reference.

In certain embodiments, the present invention provides a compound of formula I-a, wherein TBM is a TAU binding moiety

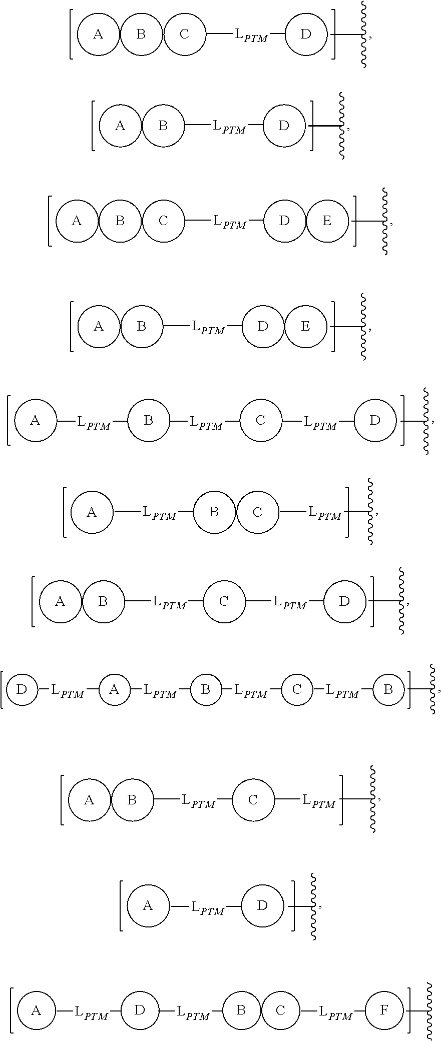

or a pharmaceutically acceptable salt thereof, wherein [symbol] denotes attachment to

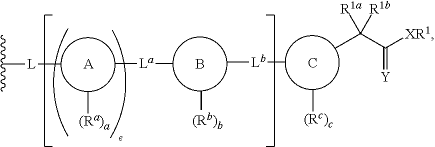

and wherein each of the variables Rings A-F and L_{PTM} is as described and defined in WO 2018/102067 and US 2018/0125821, the entirety of each of which is herein incorporated by reference.

In certain embodiments, the present invention provides a compound of formula I-a, wherein TBM is a Bcr-Abl binding moiety

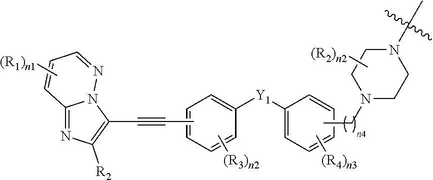

or a pharmaceutically acceptable salt thereof, wherein [symbol] denotes attachment to

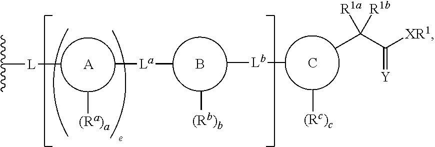

and wherein each of the variables R₁₋₅, Y₁, and n1-5 is as described and defined in WO 2018/089736, the entirety of each of which is herein incorporated by reference.

In certain embodiments, the present invention provides a compound of formula I-a, wherein TBM is an estrogen receptor binding moiety

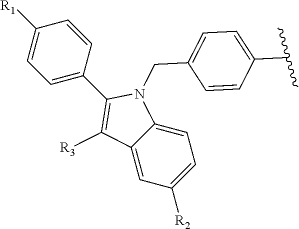

thereby forming a compound of the following formula:

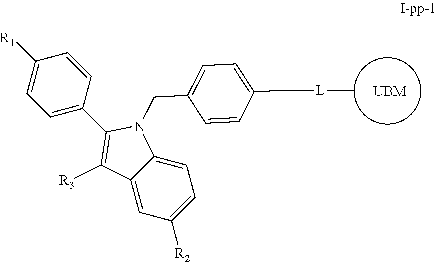

or a pharmaceutically acceptable salt thereof, wherein [symbol] denotes attachment to

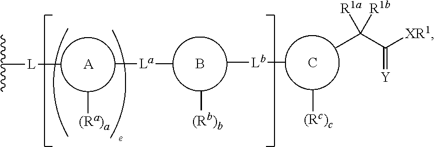

and wherein each of the variables R₁₋₃ is as described and defined in WO 2018/053354 and US 2018/072711, the entirety of each of which is herein incorporated by reference.

In certain embodiments, the present invention provides a compound of formula I-a, wherein TBM is a CDK binding moiety

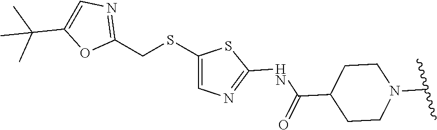 [SMILES: *N1CCC(C(=O)Nc2ncc(SCc3ncc(C(C)(C)C)o3)s2)CC1]

or a pharmaceutically acceptable salt thereof, wherein [symbol] denotes attachment to

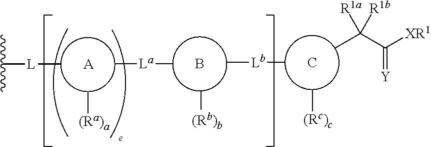

as described and defined in Olson et al., Nat. ChemBio. 2018, 14:163-170, the entirety of each of which is herein incorporated by reference.

In certain embodiments, the present invention provides a compound of formula I-a, wherein TBM is a CDK binding moiety

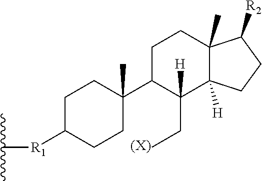

or a pharmaceutically acceptable salt thereof, wherein [symbol] denotes attachment to

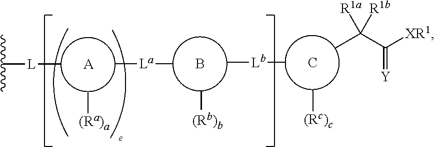

and wherein each of the variables R₁, R₂ and X is as described and defined in Hatcher et al., J. Med. Chem. 2018, 9 (6):540-545, the entirety of each of which is herein incorporated by reference.

In certain embodiments, the present invention provides a compound of formula I-a, wherein TBM is a HER binding moiety

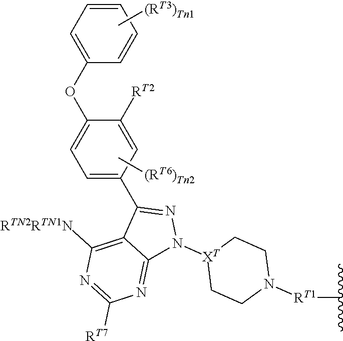

or a pharmaceutically acceptable salt thereof, wherein [symbol] denotes attachment to

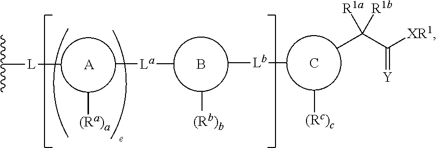

and wherein each of the variables R^{T1}, R^{T2}, R^{T3}, R^{T4}, R^{T5}, R^{T6}, R^{T7}, R^{TN1}, R^{TN2}, X^{T}, and Tn1-2 is as described and defined in WO 2017/117473, the entirety of each of which is herein incorporated by reference.

In certain embodiments, the present invention provides a compound of formula I-a, wherein TBM is a CDK4/6 binding moiety

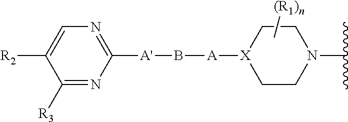

or a pharmaceutically acceptable salt thereof, wherein [symbol] denotes attachment to

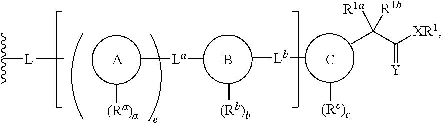

and wherein each of the variables R₁, R₂, R₃, A, A′, B, X, and n is as described and defined in WO 2017/185031 and US 2019/092768, the entirety of each of which is herein incorporated by reference.

In some embodiments, a TBM moiety is selected from PTM moieties as recited in WO 2016/197032 the entirety of which is incorporated herein by reference. In some embodiments, a TBM moiety is selected from such inhibitors as described in WO 2016/197032 the recitation of a “Linker” moiety in WO 2016/197032 corresponds to the —L— group as defined and described herein. In some embodiments, a TBM moiety is selected from such inhibitors as described in US 2018/0125821, the entirety of which is incorporated herein by reference. In some embodiments, a TBM moiety is selected from such inhibitors as described in WO 2018/119441, and US 2018/0193470, the entirety of each of which is herein incorporated by reference. In some embodiments, a TBM moiety is selected from such inhibitors as described in US 2018/0147202, the entirety of which is incorporated herein by reference. In some embodiments, a TBM moiety is selected from such inhibitors as described in WO 2018/098275 at Table A, the entirety of which is incorporated herein by reference. In some embodiments, a TBM moiety is selected from such inhibitors as described in WO 2016/169989 and US 2018/0118733, the entirety of each of which is herein incorporated by reference. In some embodiments, a TBM moiety is selected from such inhibitors as described in WO 2015/181747 and US 2017/0121335, the entirety of each of which is herein incorporated by reference. In some embodiments, a TBM moiety is selected from such inhibitors as described in Shimokawa et al., Med. Chem. Lett., 2017, 8 (10), pp 1042-1047, the entirety of which is incorporated herein by reference. In some embodiments, a TBM moiety is selected from such inhibitors as described in WO 2017/079267 and US 2018/0186785, the entirety of each of which is herein incorporated by reference. In some embodiments, a TBM moiety is selected from such inhibitors as described in Powell et al., J. Med. Chem., 2018, 61 (9), pp 4249-4255, the entirety of which is incorporated herein by reference. In some embodiments, a TBM moiety is selected from such inhibitors as described in Zhang et al., Eur. J. Med. Chem., 2018, 151, pp 304-314, the entirety of which is incorporated herein by reference. In some embodiments, a TBM moiety is selected from such inhibitors as described in Li et al., Eur. J. Med. Chem., 2018, 151, pp 237-247, the entirety of which is incorporated herein by reference. In some embodiments, a TBM moiety is selected from such inhibitors as described in WO 2016/169989 and US 2018/0118733, the entirety of each of which is herein incorporated by reference. In some embodiments, a TBM moiety is selected from such inhibitors as described in WO 2017/046036, the entirety of which is incorporated herein by reference. In some embodiments, a TBM moiety is selected from such inhibitors as described in WO 2016/169989 and US 2018/0118733, the entirety of each of which is herein incorporated by reference. In some embodiments, a TBM moiety is selected from such inhibitors as described in WO 2018/053354 and US 2018/0072711, the entirety of each of which is herein incorporated by reference. In some embodiments, a TBM moiety is selected from such inhibitors as described in Olsen et al., Nat. Chem. Bio., 2018, 14, pp 163-170, the entirety of which is incorporated herein by reference. In some embodiments, a TBM moiety is selected from such inhibitors as described in WO 2017/185031, the entirety of which is incorporated herein by reference. In some embodiments, a TBM moiety is selected from such inhibitors as described in Hatcher et al., Med. Chem. Lett., 2018, 9 (6), pp 540-545, the entirety of which is incorporated herein by reference. In some embodiments, a TBM moiety is selected from such inhibitors as described in Burslem et al., Cell Chem. Bio., 2018, 25 (1), pp 67-77, the entirety of which is incorporated herein by reference. In some embodiments, a TBM moiety is selected from such inhibitors as described in CN106977584, the entirety of which is incorporated herein by reference. In some embodiments, a TBM moiety is selected from such inhibitors as described in WO 2017/197056, the entirety of which is incorporated herein by reference. In some embodiments, a TBM moiety is selected from such inhibitors as described in WO 2018/051107, the entirety of which is incorporated herein by reference. In some embodiments, a TBM moiety is selected from such inhibitors as described in US 2018/0050021, the entirety of which is incorporated herein by reference. In some embodiments, a TBM moiety is selected from such inhibitors as described in WO 2017/223452, the entirety of which is incorporated herein by reference. In some embodiments, a TBM moiety is selected from such inhibitors as described in WO 2017/117473, WO 2017/117474, and US 2019/0016703, the entirety of each of which is herein incorporated by reference. In some embodiments, a TBM moiety is selected from such inhibitors as described in WO 2018/071606 and US 2018/0099940, the entirety of each of which is herein incorporated by reference. In some embodiments, a TBM moiety is selected from such inhibitors as described in US 2018/0099940, the entirety of which is incorporated herein by reference. In some embodiments, a TBM moiety is selected from such inhibitors as described in Gechijian et al., Nat. Chem. Bio., 2018, 14, pp. 405-412, the entirety of which is incorporated herein by reference. In some embodiments, a TBM moiety is selected from such inhibitors as described in CN 106749513, the entirety of which is incorporated herein by reference. In some embodiments, a TBM moiety is selected from such inhibitors as described in CN107056772, the entirety of which is incorporated herein by reference. In some embodiments, a TBM moiety is selected from such inhibitors as described in Pawar et al., Cell Rep., 2018, 22 (9), pp 2236-2245, the entirety of which is incorporated herein by reference. In some embodiments, a TBM moiety is selected from such inhibitors as described in US 2018/009779, the entirety of which is incorporated herein by reference. In some embodiments, a TBM moiety is selected from such inhibitors as described in WO 2017/180417, the entirety of which is incorporated herein by reference. In some embodiments, a TBM moiety is selected from such inhibitors as described in WO 2017/223452, the entirety of which is incorporated herein by reference. In some embodiments, a TBM moiety is selected from such inhibitors as described in US 2018/009779, the entirety of which is incorporated herein by reference. In some embodiments, a TBM moiety is selected from such inhibitors as described in Tomoshige et al., Bioorg. Med. Chem. Lett., 2018, 28 (4), pp 707-710, the entirety of which is incorporated herein by reference. In some embodiments, a TBM moiety is selected from such inhibitors as described in Chessum et al., J. Med. Chem., 2018, 61 (3), pp. 918-933, the entirety of which is incorporated herein by reference. In some embodiments, a TBM moiety is selected from such inhibitors as described in CN 105085620, the entirety of which is incorporated herein by reference. In some embodiments, a TBM moiety is selected from such protein target moieties as described in WO 2017/011371 and US 2017/008904, the entirety of each of which is incorporated herein by reference. In some embodiments, a TBM moiety is selected from such protein target moieties as described in US 2016/045607, the entirety of which is incorporated herein by reference. In some embodiments, a TBM moiety is selected from such protein target binders as described in US 2017/0281784, WO 2019/118893, and WO 2019/118851, the entirety of each of which is incorporated herein by reference. In some embodiments, a TBM moiety is selected from such protein target binders as described in WO 2018/144649 and US 2017/0281784, the entirety of each of which is incorporated herein by reference. In some embodiments, a TBM moiety is selected from such protein target binders as described in US 2018/0179522, WO 2018/119357, WO 2017/197056, WO 2017/011590, and US 2017/0037004, the entirety of each of which is incorporated herein by reference. In some embodiments, a TBM moiety is selected from such protein target moieties as described in WO 2017/007612 and US 2018/0134684, the entirety of each of which is incorporated herein by reference. In some embodiments, a TBM moiety is selected from such protein target moieties as described in WO 2018/064589 and U.S. Pat. No. 10,239,888, the entirety of each of which is incorporated herein by reference. In some embodiments, a TBM moiety is selected from such targeting ligands as described in U.S. Pat. No. 9,694,084, the entirety of which is incorporated herein by reference.

In some embodiments, TBM is

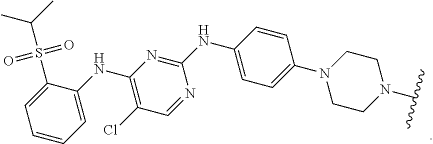 [SMILES: *N1CCN(c2ccc(Nc3ncc(Cl)c(Nc4ccccc4S(=O)(=O)C(C)C)n3)cc2)CC1]

In some embodiments, TBM is

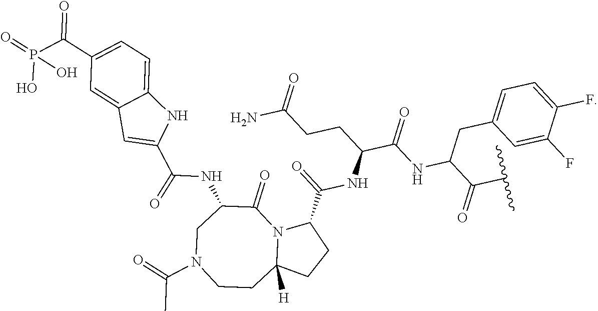 [SMILES: *C(=O)C(Cc1ccc(F)c(F)c1)NC(=O)[C@H](CCC(N)=O)NC(=O)[C@@H]1CC[C@@H]2CCN(C(C)=O)C[C@H](NC(=O)c3cc4cc(C(=O)P(=O)(O)O)ccc4[nH]3)C(=O)N21]

In some embodiments, TBM is

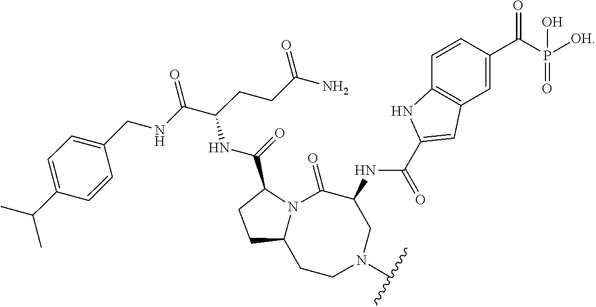 [SMILES: *N1CC[C@H]2CC[C@@H](C(=O)N[C@@H](CCC(N)=O)C(=O)NCc3ccc(C(C)C)cc3)N2C(=O)[C@@H](NC(=O)c2cc3cc(C(=O)P(=O)(O)O)ccc3[nH]2)C1]

In some embodiments, TBM is

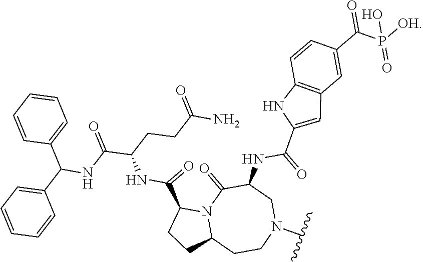 [SMILES: *N1CC[C@H]2CC[C@@H](C(=O)N[C@@H](CCC(N)=O)C(=O)NC(c3ccccc3)c3ccccc3)N2C(=O)[C@@H](NC(=O)c2cc3cc(C(=O)P(=O)(O)O)ccc3[nH]2)C1]

In some embodiments, TBM is

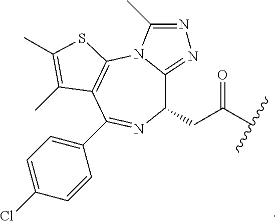 [SMILES: *C(=O)C[C@@H]1N=C(c2ccc(Cl)cc2)c2c(sc(C)c2C)-n2c(C)nnc21]

In some embodiments, TBM is

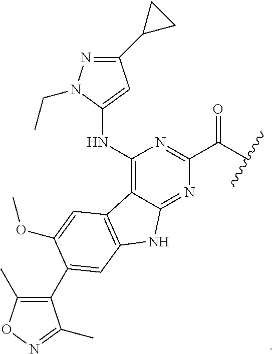 [SMILES: *C(=O)c1nc(Nc2cc(C3CC3)nn2CC)c2c(n1)[nH]c1cc(-c3c(C)noc3C)c(OC)cc12]

In some embodiments, TBM is

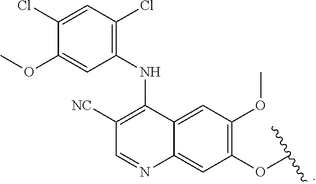 [SMILES: *Oc1cc2ncc(C#N)c(Nc3cc(OC)c(Cl)cc3Cl)c2cc1OC]

In some embodiments, TBM is

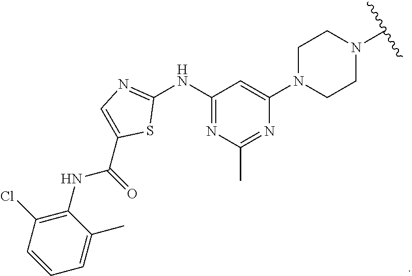 [SMILES: *N1CCN(c2cc(Nc3ncc(C(=O)Nc4c(C)cccc4Cl)s3)nc(C)n2)CC1]

In some embodiments, TBM is

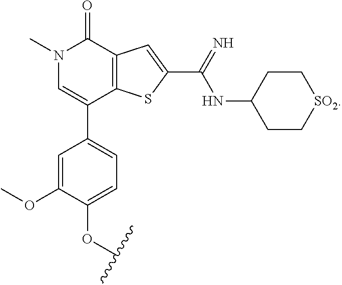 [SMILES: *Oc1ccc(-c2cn(C)c(=O)c3cc(C(=N)NC4CCS(=O)(=O)CC4)sc23)cc1OC]

In some embodiments, TBM is

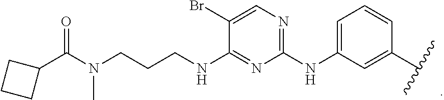 [SMILES: *c1cccc(Nc2ncc(Br)c(NCCCN(C)C(=O)C3CCC3)n2)c1]

In some embodiments, TBM is

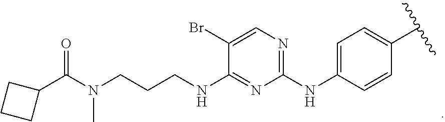 [SMILES: *c1ccc(Nc2ncc(Br)c(NCCCN(C)C(=O)C3CCC3)n2)cc1]

In some embodiments, TBM is

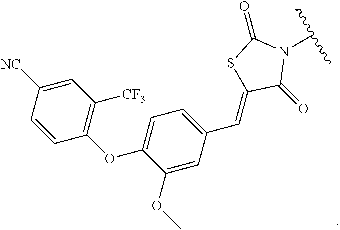 [SMILES: *N1C(=O)S/C(=C\c2ccc(Oc3ccc(C#N)cc3C(F)(F)F)c(OC)c2)C1=O]

In some embodiments, TBM is

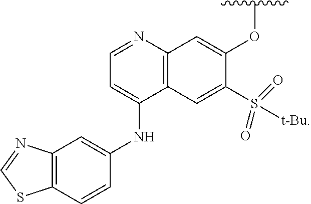 [SMILES: *Oc1cc2nccc(Nc3ccc4scnc4c3)c2cc1S(=O)(=O)C(C)(C)C]

In some embodiments, TBM is

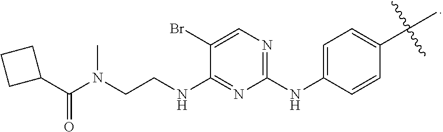 [SMILES: *c1ccc(Nc2ncc(Br)c(NCCN(C)C(=O)C3CCC3)n2)cc1]

In some embodiments, TBM is

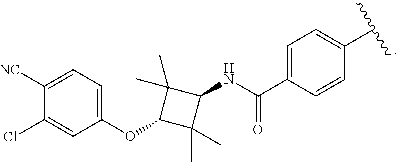 [SMILES: *c1ccc(C(=O)N[C@H]2C(C)(C)[C@H](Oc3ccc(C#N)c(Cl)c3)C2(C)C)cc1]

In some embodiments, TBM is

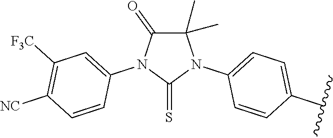 [SMILES: *c1ccc(N2C(=S)N(c3ccc(C#N)c(C(F)(F)F)c3)C(=O)C2(C)C)cc1]

In some embodiments, TBM is

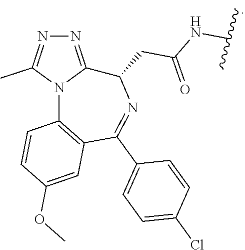 [SMILES: *NC(=O)C[C@@H]1N=C(c2ccc(Cl)cc2)c2cc(OC)ccc2-n2c(C)nnc21]

In some embodiments, TBM is

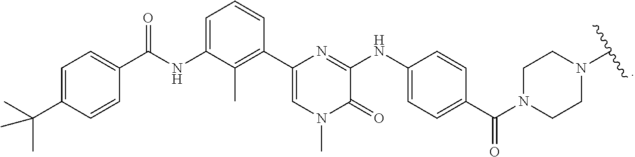 [SMILES: *N1CCN(C(=O)c2ccc(Nc3nc(-c4cccc(NC(=O)c5ccc(C(C)(C)C)cc5)c4C)cn(C)c3=O)cc2)CC1]

In some embodiments, TBM is

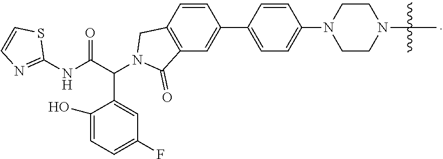 [SMILES: *N1CCN(c2ccc(-c3ccc4c(c3)C(=O)N(C(C(=O)Nc3nccs3)c3cc(F)ccc3O)C4)cc2)CC1]

In some embodiments, TBM is

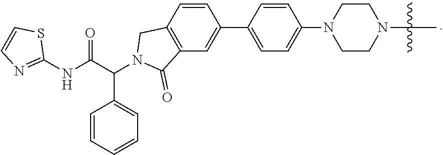 [SMILES: *N1CCN(c2ccc(-c3ccc4c(c3)C(=O)N(C(C(=O)Nc3nccs3)c3ccccc3)C4)cc2)CC1]

In some embodiments, TBM is

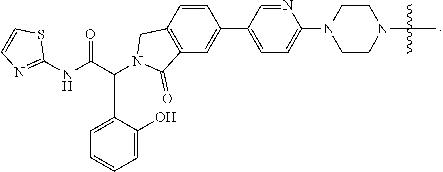 [SMILES: *N1CCN(c2ccc(-c3ccc4c(c3)C(=O)N(C(C(=O)Nc3nccs3)c3ccccc3O)C4)cn2)CC1]

In some embodiments, TBM is

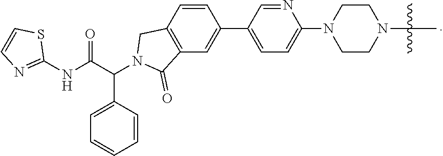 [SMILES: *N1CCN(c2ccc(-c3ccc4c(c3)C(=O)N(C(C(=O)Nc3nccs3)c3ccccc3)C4)cn2)CC1]

In some embodiments, TBM is

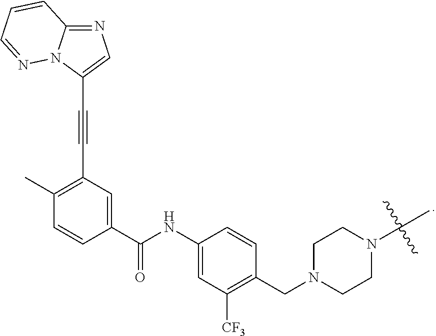 [SMILES: *N1CCN(Cc2ccc(NC(=O)c3ccc(C)c(C#Cc4cnc5cccnn45)c3)cc2C(F)(F)F)CC1]

In some embodiments, TBM is

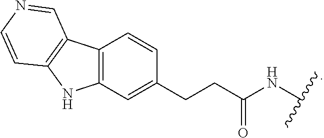 [SMILES: *NC(=O)CCc1ccc2c(c1)[nH]c1ccncc12]

In some embodiments, TBM is

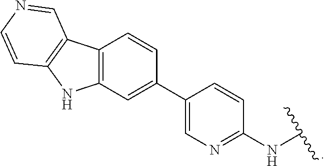 [SMILES: *Nc1ccc(-c2ccc3c(c2)[nH]c2ccncc23)cn1]

In some embodiments, TBM is

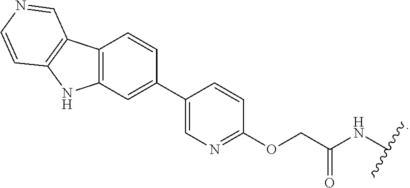 [SMILES: *NC(=O)COc1ccc(-c2ccc3c(c2)[nH]c2ccncc23)cn1]

In some embodiments, TBM is

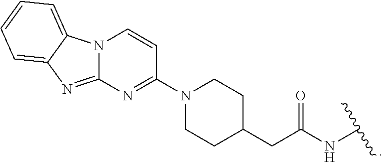 [SMILES: *NC(=O)CC1CCN(c2ccn3c(n2)nc2ccccc23)CC1]

In some embodiments, TBM is

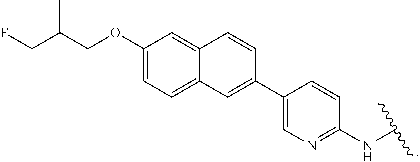 [SMILES: *Nc1ccc(-c2ccc3cc(OCC(C)CF)ccc3c2)cn1]

In some embodiments, TBM is

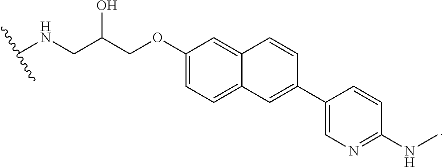 [SMILES: *NCC(O)COc1ccc2cc(-c3ccc(NC)nc3)ccc2c1]

In some embodiments, TBM is

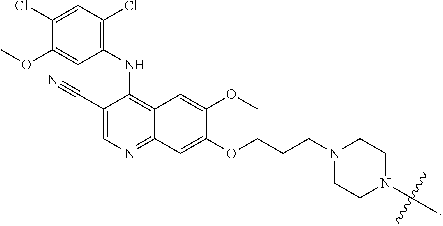 [SMILES: *N1CCN(CCCOc2cc3ncc(C#N)c(Nc4cc(OC)c(Cl)cc4Cl)c3cc2OC)CC1]

In some embodiments, TBM is

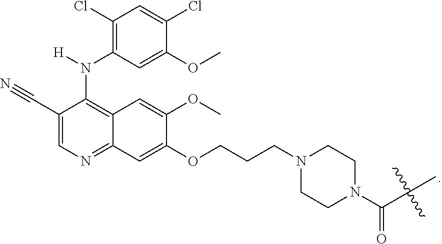 [SMILES: *C(=O)N1CCN(CCCOc2cc3ncc(C#N)c(Nc4cc(OC)c(Cl)cc4Cl)c3cc2OC)CC1]

In some embodiments, TBM is

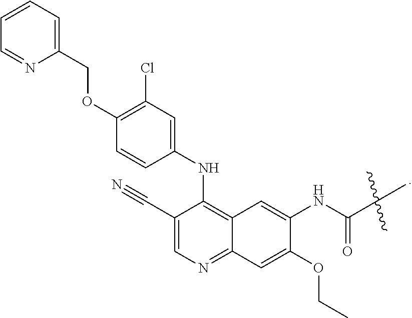 [SMILES: *C(=O)Nc1cc2c(Nc3ccc(OCc4ccccn4)c(Cl)c3)c(C#N)cnc2cc1OCC]

In some embodiments, TBM is

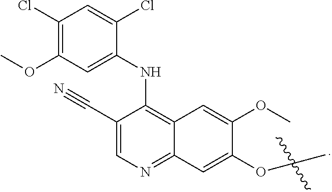 [SMILES: *Oc1cc2ncc(C#N)c(Nc3cc(OC)c(Cl)cc3Cl)c2cc1OC]

In some embodiments, TBM is

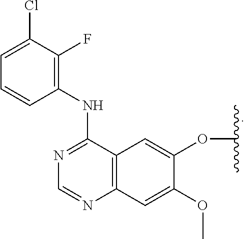 [SMILES: *Oc1cc2c(Nc3cccc(Cl)c3F)ncnc2cc1OC]

In some embodiments, TBM is

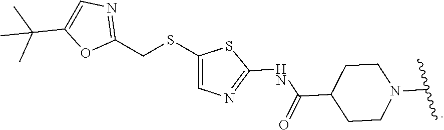 [SMILES: *N1CCC(C(=O)Nc2ncc(SCc3ncc(C(C)(C)C)o3)s2)CC1]

In some embodiments, TBM is

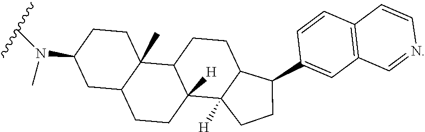

In some embodiments, TBM is

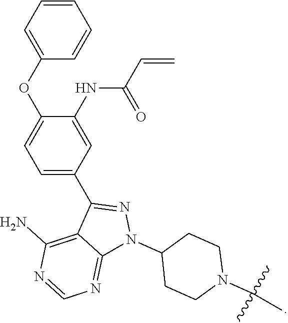 [SMILES: *N1CCC(n2nc(-c3ccc(Oc4ccccc4)c(NC(=O)C=C)c3)c3c(N)ncnc32)CC1]

In some embodiments, TBM is

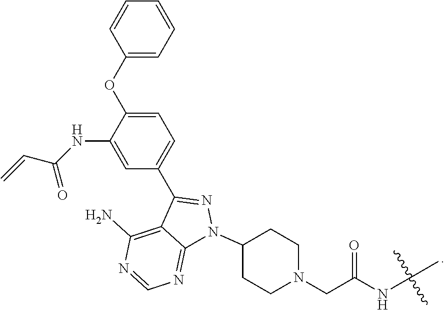 [SMILES: *NC(=O)CN1CCC(n2nc(-c3ccc(Oc4ccccc4)c(NC(=O)C=C)c3)c3c(N)ncnc32)CC1]

In some embodiments, TBM is

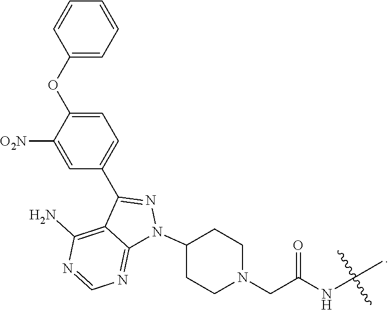 [SMILES: *NC(=O)CN1CCC(n2nc(-c3ccc(Oc4ccccc4)c([N+](=O)[O-])c3)c3c(N)ncnc32)CC1]

In some embodiments, TBM is

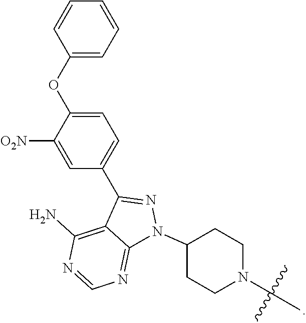 [SMILES: *N1CCC(n2nc(-c3ccc(Oc4ccccc4)c([N+](=O)[O-])c3)c3c(N)ncnc32)CC1]

In some embodiments, TBM is

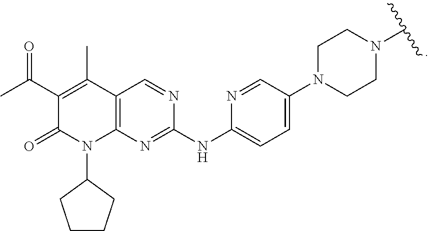 [SMILES: *N1CCN(c2ccc(Nc3ncc4c(C)c(C(C)=O)c(=O)n(C5CCCC5)c4n3)nc2)CC1]

In some embodiments, TBM is

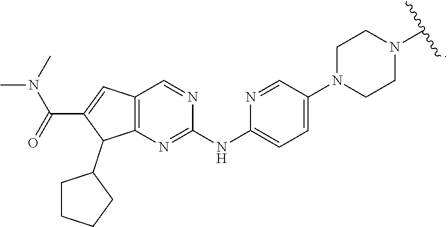 [SMILES: *N1CCN(c2ccc(Nc3ncc4c(n3)C(C3CCCC3)C(C(=O)N(C)C)=C4)nc2)CC1]

In some embodiments, TBM is

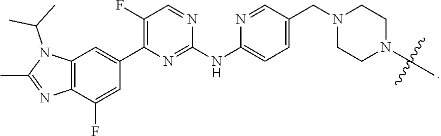 [SMILES: *N1CCN(Cc2ccc(Nc3ncc(F)c(-c4cc(F)c5nc(C)n(C(C)C)c5c4)n3)nc2)CC1]

In some embodiments, TBM is

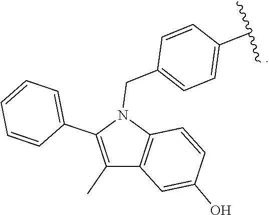 [SMILES: *c1ccc(Cn2c(-c3ccccc3)c(C)c3cc(O)ccc32)cc1]

In some embodiments, TBM is

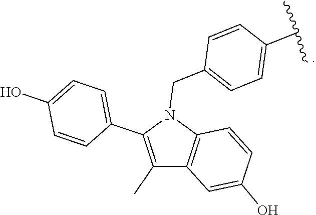 [SMILES: *c1ccc(Cn2c(-c3ccc(O)cc3)c(C)c3cc(O)ccc32)cc1]

In some embodiments, TBM is

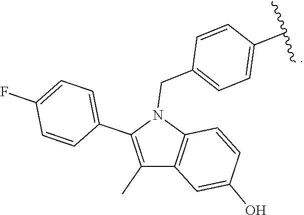 [SMILES: *c1ccc(Cn2c(-c3ccc(F)cc3)c(C)c3cc(O)ccc32)cc1]

In some embodiments, TBM is

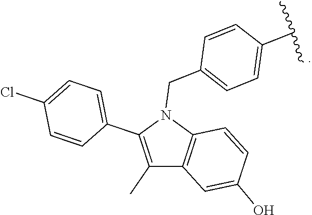 [SMILES: *c1ccc(Cn2c(-c3ccc(Cl)cc3)c(C)c3cc(O)ccc32)cc1]

In some embodiments, TBM is

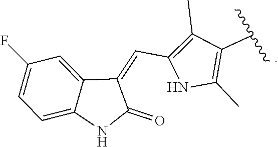 [SMILES: *c1c(C)[nH]c(/C=C2\C(=O)Nc3ccc(F)cc32)c1C]

In some embodiments, TBM is

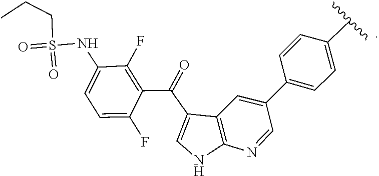 [SMILES: *c1ccc(-c2cnc3[nH]cc(C(=O)c4c(F)ccc(NS(=O)(=O)CCC)c4F)c3c2)cc1]

In some embodiments, TBM is

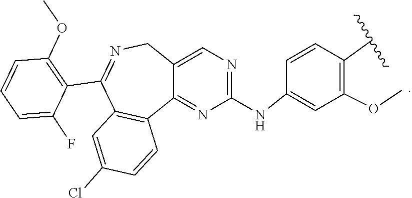 [SMILES: *c1ccc(Nc2ncc3c(n2)-c2ccc(Cl)cc2C(c2c(F)cccc2OC)=NC3)cc1OC]

In some embodiments, TBM is

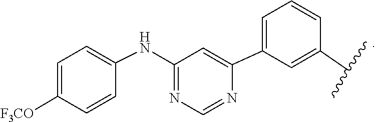 [SMILES: *c1cccc(-c2cc(Nc3ccc(OC(F)(F)F)cc3)ncn2)c1]

In some embodiments, TBM is

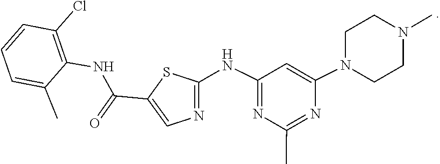 [SMILES: Cc1nc(Nc2ncc(C(=O)Nc3c(C)cccc3Cl)s2)cc(N2CCN(C)CC2)n1]

In some embodiments, TBM is

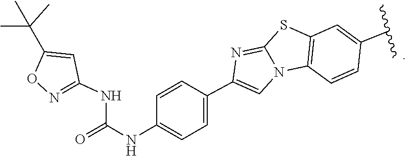 [SMILES: *c1ccc2c(c1)sc1nc(-c3ccc(NC(=O)Nc4cc(C(C)(C)C)on4)cc3)cn12]

In some embodiments, TBM is

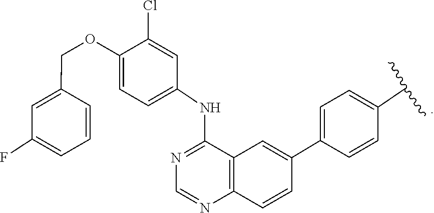 [SMILES: *c1ccc(-c2ccc3ncnc(Nc4ccc(OCc5cccc(F)c5)c(Cl)c4)c3c2)cc1]

In some embodiment, TBM is selected from the compounds listed in Table 1B.

In some embodiments, a provided compound or pharmaceutically acceptable salt thereof, is selected from those wherein KBM is

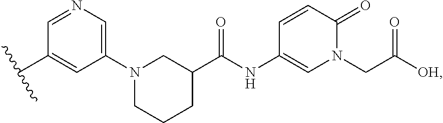 [SMILES: *c1cncc(N2CCCC(C(=O)Nc3ccc(=O)n(CC(=O)O)c3)C2)c1]

TBM is selected from but not limited to any of those in Table A below, and L is selected from any of those in Table B below.

In some embodiments, a provided compound or pharmaceutically acceptable salt thereof, is selected from those wherein KBM is

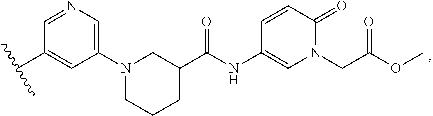 [SMILES: *c1cncc(N2CCCC(C(=O)Nc3ccc(=O)n(CC(=O)OC)c3)C2)c1]

TBM is selected from but not limited to any of those in Table A below, and L is selected from any of those in Table B below.

In some embodiments, a provided compound or pharmaceutically acceptable salt thereof, is selected from those wherein KBM is

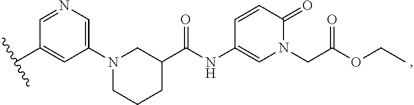 [SMILES: *c1cncc(N2CCCC(C(=O)Nc3ccc(=O)n(CC(=O)OCC)c3)C2)c1]

TBM is selected from but not limited to any of those in Table A below, and L is selected from any of those in Table B below.

In some embodiments, a provided compound or pharmaceutically acceptable salt thereof, is selected from those wherein KBM is

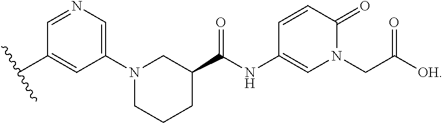 [SMILES: *c1cncc(N2CCC[C@H](C(=O)Nc3ccc(=O)n(CC(=O)O)c3)C2)c1]

TBM is selected from but not limited to any of those in Table A below, and L is selected from any of those in Table B below.

In some embodiments, a provided compound or pharmaceutically acceptable salt thereof, is selected from those wherein KBM is

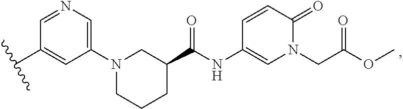 [SMILES: *c1cncc(N2CCC[C@H](C(=O)Nc3ccc(=O)n(CC(=O)OC)c3)C2)c1]

TBM is selected from but not limited to any of those in Table A below, and L is selected from any of those in Table B below.

In some embodiments, a provided compound or pharmaceutically acceptable salt thereof, is selected from those wherein KBM is

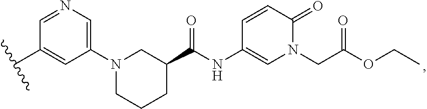 [SMILES: *c1cncc(N2CCC[C@H](C(=O)Nc3ccc(=O)n(CC(=O)OCC)c3)C2)c1]

TBM is selected from but not limited to any of those in Table A below, and L is selected from any of those in Table B below.

In some embodiments, a provided compound or pharmaceutically acceptable salt thereof, is selected from those wherein KBM is

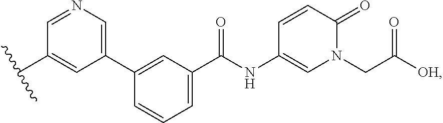 [SMILES: *c1cncc(-c2cccc(C(=O)Nc3ccc(=O)n(CC(=O)O)c3)c2)c1]

TBM is selected from but not limited to any of those in Table A below, and L is selected from any of those in Table B below.

In some embodiments, a provided compound or pharmaceutically acceptable salt thereof, is selected from those wherein KBM is

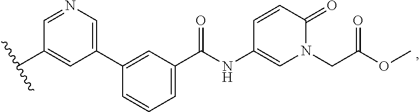 [SMILES: *c1cncc(-c2cccc(C(=O)Nc3ccc(=O)n(CC(=O)OC)c3)c2)c1]

TBM is selected from but not limited to any of those in Table A below, and L is selected from any of those in Table B below.

In some embodiments, a provided compound or pharmaceutically acceptable salt thereof, is selected from those wherein KBM is

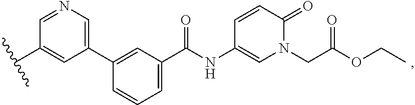 [SMILES: *c1cncc(-c2cccc(C(=O)Nc3ccc(=O)n(CC(=O)OCC)c3)c2)c1]

TBM is selected from but not limited to any of those in Table A below, and L is selected from any of those in Table B below.

In some embodiments, a provided compound or pharmaceutically acceptable salt thereof, is selected from those wherein KBM is

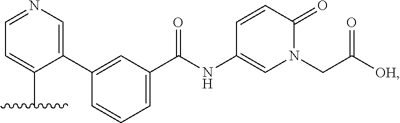 [SMILES: *c1ccncc1-c1cccc(C(=O)Nc2ccc(=O)n(CC(=O)O)c2)c1]

TBM is selected from but not limited to any of those in Table A below, and L is selected from any of those in Table B below.

In some embodiments, a provided compound or pharmaceutically acceptable salt thereof, is selected from those wherein KBM is

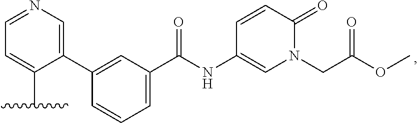 [SMILES: *c1ccncc1-c1cccc(C(=O)Nc2ccc(=O)n(CC(=O)OC)c2)c1]

TBM is selected from but not limited to any of those in Table A below, and L is selected from any of those in Table B below.

In some embodiments, a provided compound or pharmaceutically acceptable salt thereof, is selected from those wherein KBM is

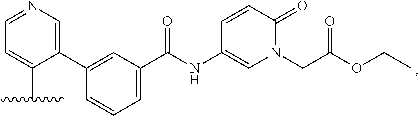 [SMILES: *c1ccncc1-c1cccc(C(=O)Nc2ccc(=O)n(CC(=O)OCC)c2)c1]

TBM is selected from but not limited to any of those in Table A below, and L is selected from any of those in Table B below.

In some embodiments, a provided compound or pharmaceutically acceptable salt thereof, is selected from those wherein KBM is

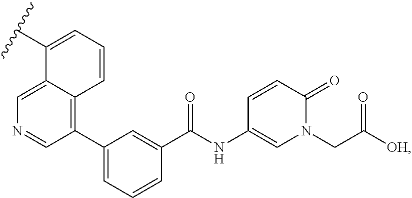 [SMILES: *c1cccc2c(-c3cccc(C(=O)Nc4ccc(=O)n(CC(=O)O)c4)c3)cncc12]

TBM is selected from but not limited to any of those in Table A below, and L is selected from any of those in Table B below.

In some embodiments, a provided compound or pharmaceutically acceptable salt thereof, is selected from those wherein KBM is

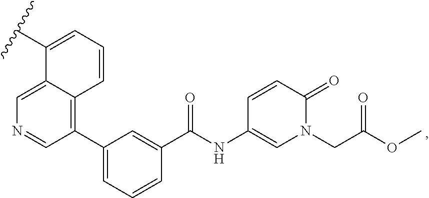 [SMILES: *c1cccc2c(-c3cccc(C(=O)Nc4ccc(=O)n(CC(=O)OC)c4)c3)cncc12]

TBM is selected from but not limited to any of those in Table A below, and L is selected from any of those in Table B below.

In some embodiments, a provided compound or pharmaceutically acceptable salt thereof, is selected from those wherein KBM is

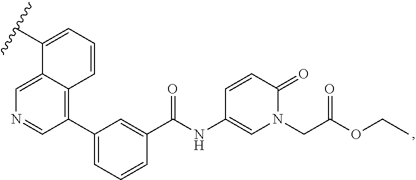 [SMILES: *c1cccc2c(-c3cccc(C(=O)Nc4ccc(=O)n(CC(=O)OCC)c4)c3)cncc12]

TBM is selected from but not limited to any of those in Table A below, and L is selected from any of those in Table B below.

In some embodiments, a provided compound or pharmaceutically acceptable salt thereof, is selected from those wherein KBM is

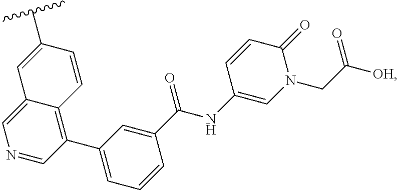 [SMILES: *c1ccc2c(-c3cccc(C(=O)Nc4ccc(=O)n(CC(=O)O)c4)c3)cncc2c1]

TBM is selected from but not limited to any of those in Table A below, and L is selected from any of those in Table B below.

In some embodiments, a provided compound or pharmaceutically acceptable salt thereof, is selected from those wherein KBM is

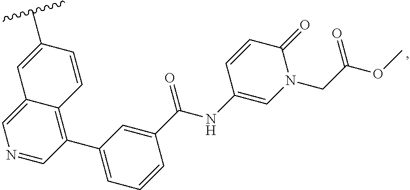 [SMILES: *c1ccc2c(-c3cccc(C(=O)Nc4ccc(=O)n(CC(=O)OC)c4)c3)cncc2c1]

TBM is selected from but not limited to any of those in Table A below, and L is selected from any of those in Table B below.

In some embodiments, a provided compound or pharmaceutically acceptable salt thereof, is selected from those wherein KBM is

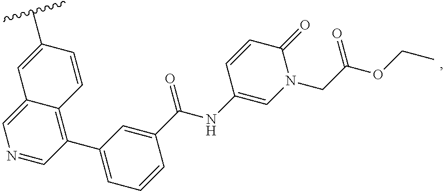 [SMILES: *c1ccc2c(-c3cccc(C(=O)Nc4ccc(=O)n(CC(=O)OCC)c4)c3)cncc2c1]

TBM is selected from but not limited to any of those in Table A below, and L is selected from any of those in Table B below.

In some embodiments, a provided compound or pharmaceutically acceptable salt thereof, is selected from those wherein KBM is

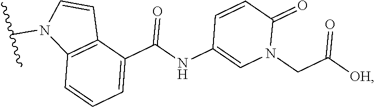 [SMILES: *n1ccc2c(C(=O)Nc3ccc(=O)n(CC(=O)O)c3)cccc21]

TBM is selected from but not limited to any of those in Table A below, and L is selected from any of those in Table B below.

In some embodiments, a provided compound or pharmaceutically acceptable salt thereof, is selected from those wherein KBM is

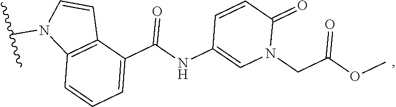 [SMILES: *n1ccc2c(C(=O)Nc3ccc(=O)n(CC(=O)OC)c3)cccc21]

TBM is selected from but not limited to any of those in Table A below, and L is selected from any of those in Table B below.

In some embodiments, a provided compound or pharmaceutically acceptable salt thereof, is selected from those wherein KBM is

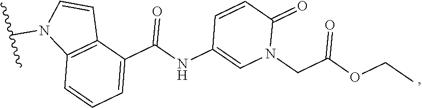 [SMILES: *n1ccc2c(C(=O)Nc3ccc(=O)n(CC(=O)OCC)c3)cccc21]

TBM is selected from but not limited to any of those in Table A below, and L is selected from any of those in Table B below.

In some embodiments, a provided compound or pharmaceutically acceptable salt thereof, is selected from those wherein KBM is

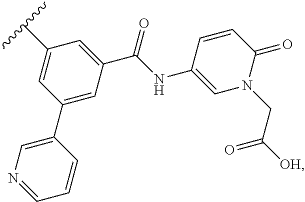 [SMILES: *c1cc(C(=O)Nc2ccc(=O)n(CC(=O)O)c2)cc(-c2cccnc2)c1]

TBM is selected from but not limited to any of those in Table A below, and L is selected from any of those in Table B below.

In some embodiments, a provided compound or pharmaceutically acceptable salt thereof, is selected from those wherein KBM is

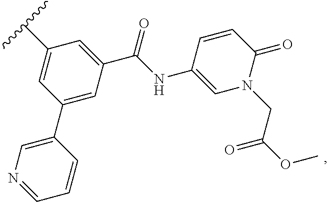 [SMILES: *c1cc(C(=O)Nc2ccc(=O)n(CC(=O)OC)c2)cc(-c2cccnc2)c1]

TBM is selected from but not limited to any of those in Table A below, and L is selected from any of those in Table B below.

In some embodiments, a provided compound or pharmaceutically acceptable salt thereof, is selected from those wherein KBM is

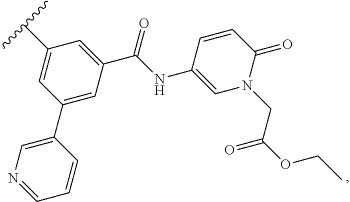 [SMILES: *c1cc(C(=O)Nc2ccc(=O)n(CC(=O)OCC)c2)cc(-c2cccnc2)c1]

TBM is selected from but not limited to any of those in Table A below, and L is selected from any of those in Table B below.

In some embodiments, a provided compound or pharmaceutically acceptable salt thereof, is selected from those wherein KBM is

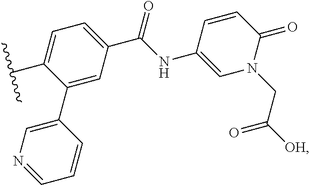 [SMILES: *c1ccc(C(=O)Nc2ccc(=O)n(CC(=O)O)c2)cc1-c1cccnc1]

TBM is selected from but not limited to any of those in Table A below, and L is selected from any of those in Table B below.

In some embodiments, a provided compound or pharmaceutically acceptable salt thereof, is selected from those wherein KBM is

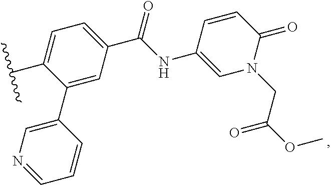 [SMILES: *c1ccc(C(=O)Nc2ccc(=O)n(CC(=O)OC)c2)cc1-c1cccnc1]

TBM is selected from but not limited to any of those in Table A below, and L is selected from any of those in Table B below.

In some embodiments, a provided compound or pharmaceutically acceptable salt thereof, is selected from those wherein KBM is

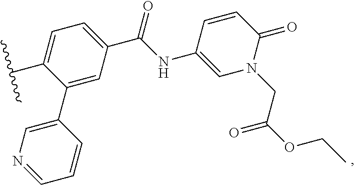 [SMILES: *c1ccc(C(=O)Nc2ccc(=O)n(CC(=O)OCC)c2)cc1-c1cccnc1]

TBM is selected from but not limited to any of those in Table A below, and L is selected from any of those in Table B below.

In some embodiments, a provided compound or pharmaceutically acceptable salt thereof, is selected from those wherein KBM is

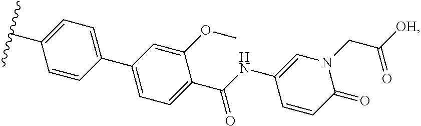 [SMILES: *c1ccc(-c2ccc(C(=O)Nc3ccc(=O)n(CC(=O)O)c3)c(OC)c2)cc1]

TBM is selected from but not limited to any of those in Table A below, and L is selected from any of those in Table B below.

In some embodiments, a provided compound or pharmaceutically acceptable salt thereof, is selected from those wherein KBM is

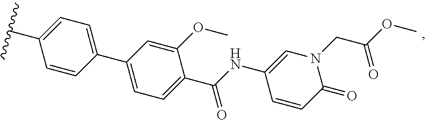 [SMILES: *c1ccc(-c2ccc(C(=O)Nc3ccc(=O)n(CC(=O)OC)c3)c(OC)c2)cc1]

TBM is selected from but not limited to any of those in Table A below, and L is selected from any of those in Table B below.

In some embodiments, a provided compound or pharmaceutically acceptable salt thereof, is selected from those wherein KBM is

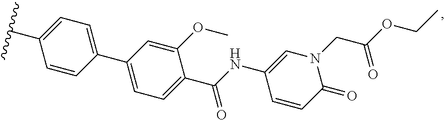 [SMILES: *c1ccc(-c2ccc(C(=O)Nc3ccc(=O)n(CC(=O)OCC)c3)c(OC)c2)cc1]

TBM is selected from but not limited to any of those in Table A below, and L is selected from any of those in Table B below.

In some embodiments, a provided compound or pharmaceutically acceptable salt thereof, is selected from those wherein KBM is

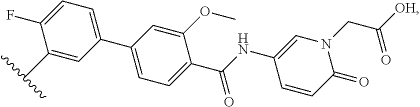 [SMILES: *c1cc(-c2ccc(C(=O)Nc3ccc(=O)n(CC(=O)O)c3)c(OC)c2)ccc1F]

TBM is selected from but not limited to any of those in Table A below, and L is selected from any of those in Table B below.

In some embodiments, a provided compound or pharmaceutically acceptable salt thereof, is selected from those wherein KBM is

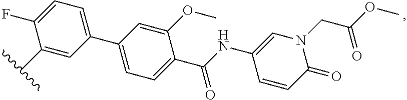 [SMILES: *c1cc(-c2ccc(C(=O)Nc3ccc(=O)n(CC(=O)OC)c3)c(OC)c2)ccc1F]

TBM is selected from but not limited to any of those in Table A below, and L is selected from any of those in Table B below.

In some embodiments, a provided compound or pharmaceutically acceptable salt thereof, is selected from those wherein KBM is

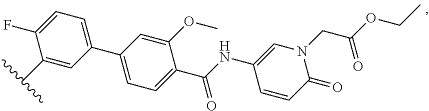 [SMILES: *c1cc(-c2ccc(C(=O)Nc3ccc(=O)n(CC(=O)OCC)c3)c(OC)c2)ccc1F]

TBM is selected from but not limited to any of those in Table A below, and L is selected from any of those in Table B below.

In some embodiments, a provided compound or pharmaceutically acceptable salt thereof, is selected from those wherein KBM is

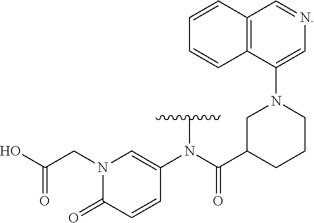 [SMILES: *N(C(=O)C1CCCN(c2cncc3ccccc23)C1)c1ccc(=O)n(CC(=O)O)c1]

TBM is selected from but not limited to any of those in Table A below, and L is selected from any of those in Table B below.

TABLE A
Exemplified Binders (TBM)

| | |
|---|---|
| 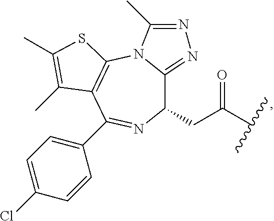 [SMILES: *C(=O)C[C@@H]1N=C(c2ccc(Cl)cc2)c2c(sc(C)c2C)-n2c(C)nnc21] | (a) |
| 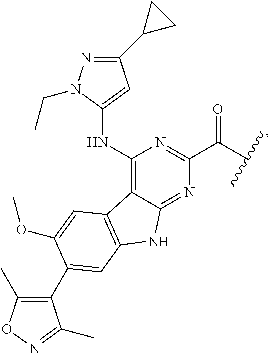 [SMILES: *C(=O)c1nc(Nc2cc(C3CC3)nn2CC)c2c(n1)[nH]c1cc(-c3c(C)noc3C)c(OC)cc12] | (b) |
| 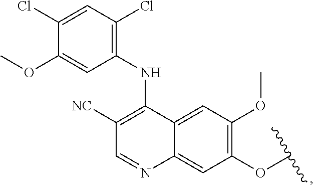 [SMILES: *Oc1cc2ncc(C#N)c(Nc3cc(OC)c(Cl)cc3Cl)c2cc1OC] | (c) |
| 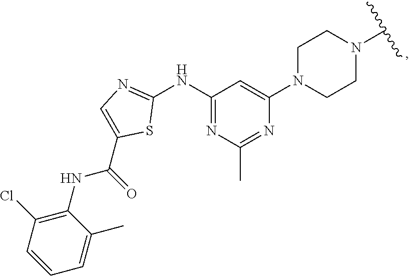 [SMILES: *N1CCN(c2cc(Nc3ncc(C(=O)Nc4c(C)cccc4Cl)s3)nc(C)n2)CC1] | (d) |
| 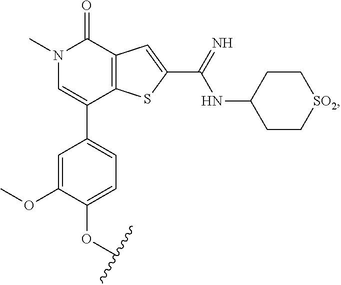 [SMILES: *Oc1ccc(-c2cn(C)c(=O)c3cc(C(=N)NC4CCS(=O)(=O)CC4)sc23)cc1OC] | (e) |
| 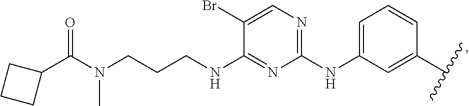 [SMILES: *c1cccc(Nc2ncc(Br)c(NCCCN(C)C(=O)C3CCC3)n2)c1] | (h) |
| 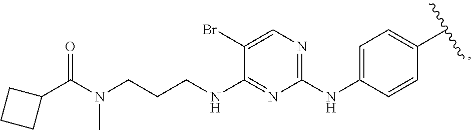 [SMILES: *c1ccc(Nc2ncc(Br)c(NCCCN(C)C(=O)C3CCC3)n2)cc1] | (i) |
| 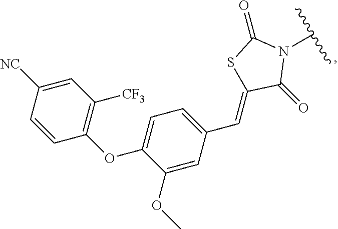 [SMILES: *N1C(=O)S/C(=C\c2ccc(Oc3ccc(C#N)cc3C(F)(F)F)c(OC)c2)C1=O] | (j) |
| 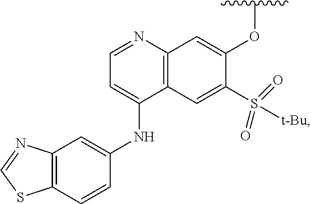 [SMILES: *Oc1cc2nccc(Nc3ccc4scnc4c3)c2cc1S(=O)(=O)C(C)(C)C] | (k) |
| 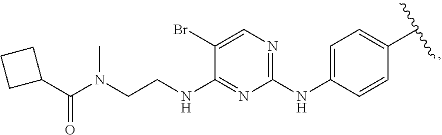 [SMILES: *c1ccc(Nc2ncc(Br)c(NCCN(C)C(=O)C3CCC3)n2)cc1] | (l) |
| 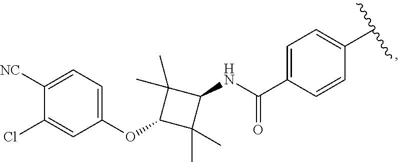 [SMILES: *c1ccc(C(=O)N[C@H]2C(C)(C)[C@H](Oc3ccc(C#N)c(Cl)c3)C2(C)C)cc1] | (m) |
| 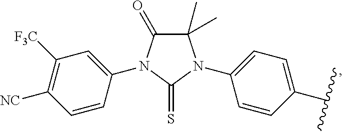 [SMILES: *c1ccc(N2C(=S)N(c3ccc(C#N)c(C(F)(F)F)c3)C(=O)C2(C)C)cc1] | (n) |
| 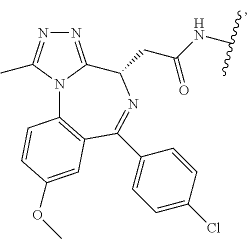 [SMILES: *NC(=O)C[C@@H]1N=C(c2ccc(Cl)cc2)c2cc(OC)ccc2-n2c(C)nnc21] | (o) |
| 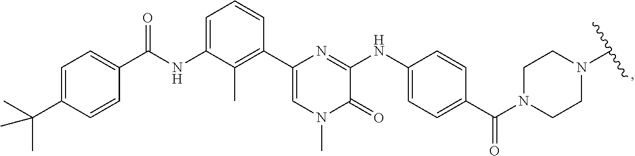 [SMILES: *N1CCN(C(=O)c2ccc(Nc3nc(-c4cccc(NC(=O)c5ccc(C(C)(C)C)cc5)c4C)cn(C)c3=O)cc2)CC1] | (p) |
| 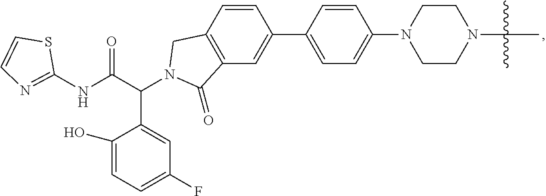 [SMILES: *N1CCN(c2ccc(-c3ccc4c(c3)C(=O)N(C(C(=O)Nc3nccs3)c3cc(F)ccc3O)C4)cc2)CC1] | (q) |
| 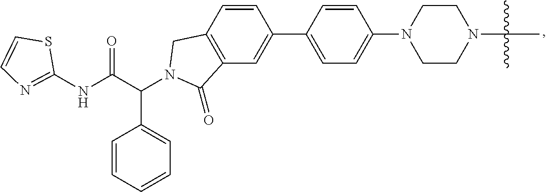 [SMILES: *N1CCN(c2ccc(-c3ccc4c(c3)C(=O)N(C(C(=O)Nc3nccs3)c3ccccc3)C4)cc2)CC1] | (r) |
| 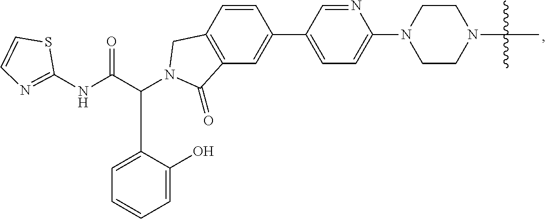 [SMILES: *N1CCN(c2ccc(-c3ccc4c(c3)C(=O)N(C(C(=O)Nc3nccs3)c3ccccc3O)C4)cn2)CC1] | (s) |
| 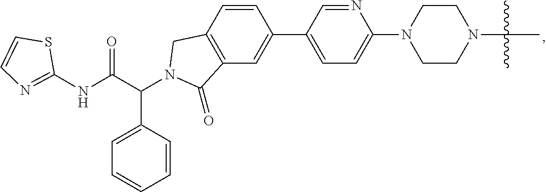 [SMILES: *N1CCN(c2ccc(-c3ccc4c(c3)C(=O)N(C(C(=O)Nc3nccs3)c3ccccc3)C4)cn2)CC1] | (t) |
| 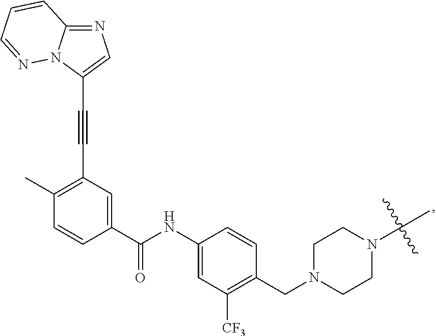 [SMILES: *N1CCN(Cc2ccc(NC(=O)c3ccc(C)c(C#Cc4cnc5cccnn45)c3)cc2C(F)(F)F)CC1] | (u) |
| 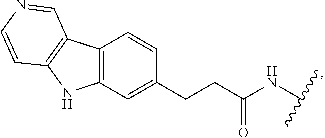 [SMILES: *NC(=O)CCc1ccc2c(c1)[nH]c1ccncc12] | (v) |
| 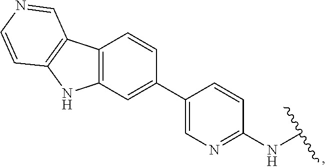 [SMILES: *Nc1ccc(-c2ccc3c(c2)[nH]c2ccncc23)cn1] | (w) |
| 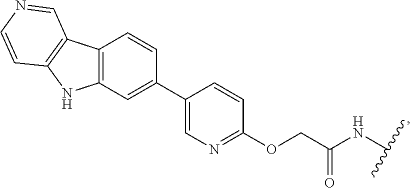 [SMILES: *NC(=O)COc1ccc(-c2ccc3c(c2)[nH]c2ccncc23)cn1] | (x) |
| 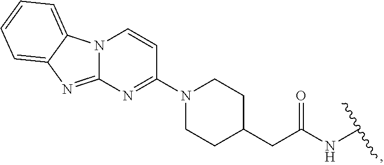 [SMILES: *NC(=O)CC1CCN(c2ccn3c(n2)nc2ccccc23)CC1] | (y) |
| 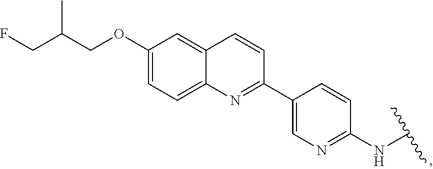 [SMILES: *Nc1ccc(-c2ccc3cc(OCC(C)CF)ccc3n2)cn1] | (z) |
| 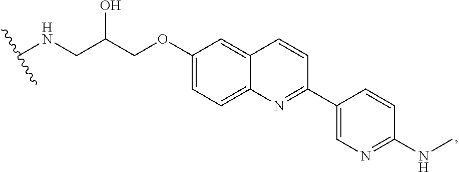 [SMILES: *NCC(O)COc1ccc2nc(-c3ccc(NC)nc3)ccc2c1] | (aa) |
| 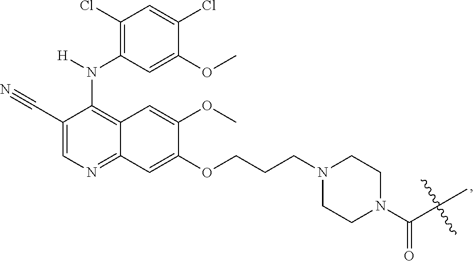 [SMILES: *C(=O)N1CCN(CCCOc2cc3ncc(C#N)c(Nc4cc(OC)c(Cl)cc4Cl)c3cc2OC)CC1] | (bb) |
| 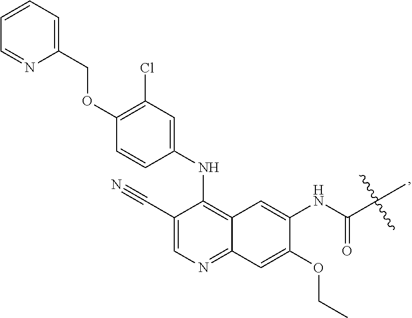 [SMILES: *C(=O)Nc1cc2c(Nc3ccc(OCc4ccccn4)c(Cl)c3)c(C#N)cnc2cc1OCC] | (cc) |
| 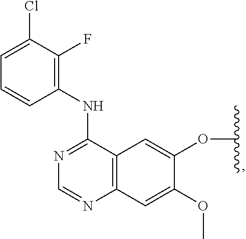 [SMILES: *Oc1cc2c(Nc3cccc(Cl)c3F)ncnc2cc1OC] | (dd) |
| 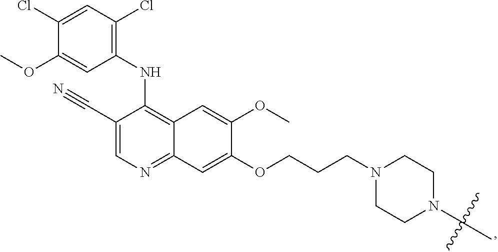 [SMILES: *N1CCN(CCCOc2cc3ncc(C#N)c(Nc4cc(OC)c(Cl)cc4Cl)c3cc2OC)CC1] | (ee) |
| 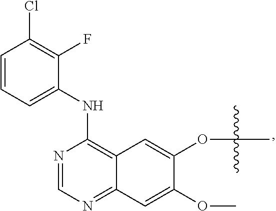 [SMILES: *Oc1cc2c(Nc3cccc(Cl)c3F)ncnc2cc1OC] | (ff) |
| 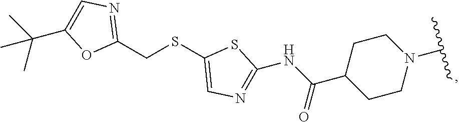 [SMILES: *N1CCC(C(=O)Nc2ncc(SCc3ncc(C(C)(C)C)o3)s2)CC1] | (gg) |
| 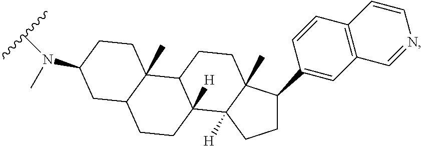 | (hh) |
| 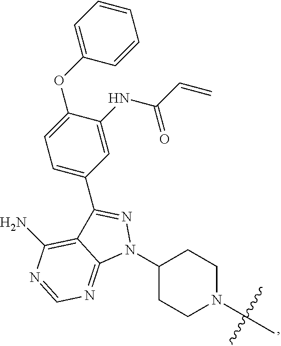 [SMILES: *N1CCC(n2nc(-c3ccc(Oc4ccccc4)c(NC(=O)C=C)c3)c3c(N)ncnc32)CC1] | (ii) |
| 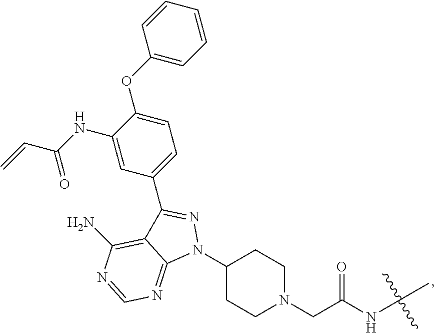 [SMILES: *NC(=O)CN1CCC(n2nc(-c3ccc(Oc4ccccc4)c(NC(=O)C=C)c3)c3c(N)ncnc32)CC1] | (jj) |
| 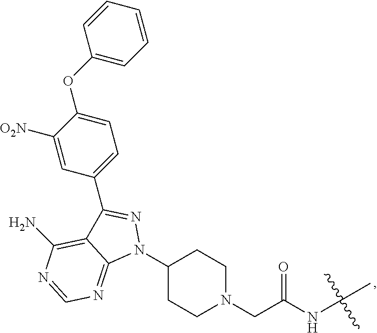 [SMILES: *NC(=O)CN1CCC(n2nc(-c3ccc(Oc4ccccc4)c([N+](=O)[O-])c3)c3c(N)ncnc32)CC1] | (kk) |
| 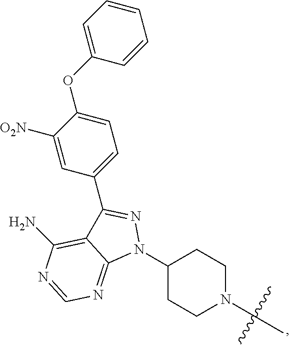 [SMILES: *N1CCC(n2nc(-c3ccc(Oc4ccccc4)c([N+](=O)[O-])c3)c3c(N)ncnc32)CC1] | (ll) |
| 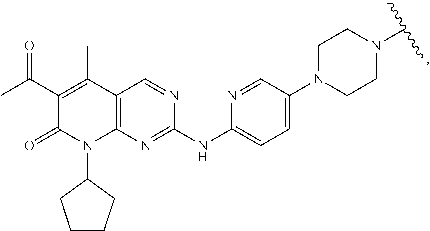 [SMILES: *N1CCN(c2ccc(Nc3ncc4c(C)c(C(C)=O)c(=O)n(C5CCCC5)c4n3)nc2)CC1] | (mm) |
| 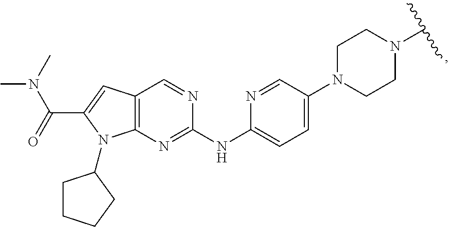 [SMILES: *N1CCN(c2ccc(Nc3ncc4cc(C(=O)N(C)C)n(C5CCCC5)c4n3)nc2)CC1] | (nn) |
| 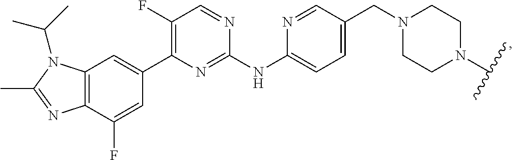 [SMILES: *N1CCN(Cc2ccc(Nc3ncc(F)c(-c4cc(F)c5nc(C)n(C(C)C)c5c4)n3)nc2)CC1] | (oo) |
| 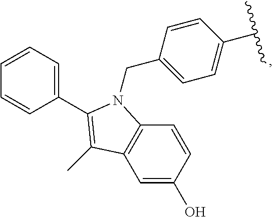 [SMILES: *c1ccc(Cn2c(-c3ccccc3)c(C)c3cc(O)ccc32)cc1] | (pp) |
| 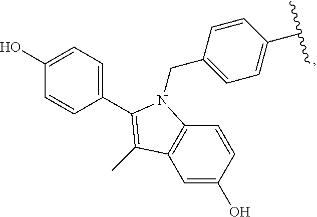 [SMILES: *c1ccc(Cn2c(-c3ccc(O)cc3)c(C)c3cc(O)ccc32)cc1] | (qq) |
| 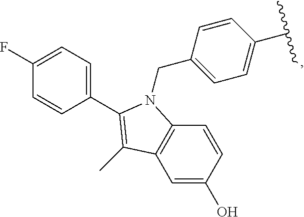 [SMILES: *c1ccc(Cn2c(-c3ccc(F)cc3)c(C)c3cc(O)ccc32)cc1] | (rr) |
| 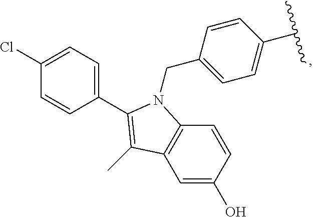 [SMILES: *c1ccc(Cn2c(-c3ccc(Cl)cc3)c(C)c3cc(O)ccc32)cc1] | (ss) |
| 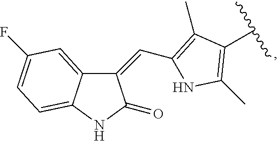 [SMILES: *c1c(C)[nH]c(/C=C2\C(=O)Nc3ccc(F)cc32)c1C] | (tt) |
| 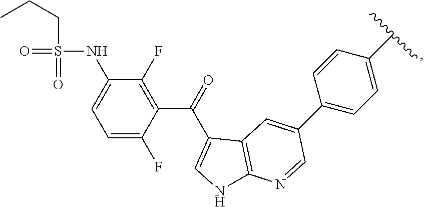 [SMILES: *c1ccc(-c2cnc3[nH]cc(C(=O)c4c(F)ccc(NS(=O)(=O)CCC)c4F)c3c2)cc1] | (uu) |
| 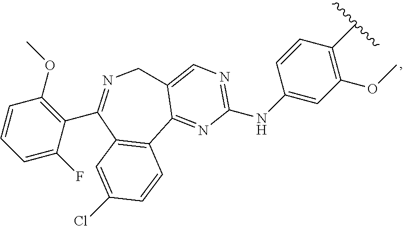 [SMILES: *c1ccc(Nc2ncc3c(n2)-c2ccc(Cl)cc2C(c2c(F)cccc2OC)=NC3)cc1OC] | (vv) |
| 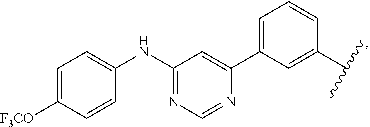 [SMILES: *c1cccc(-c2cc(Nc3ccc(OC(F)(F)F)cc3)ncn2)c1] | (ww) |
| 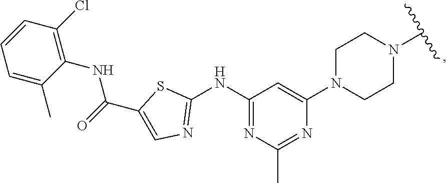 [SMILES: *N1CCN(c2cc(Nc3ncc(C(=O)Nc4c(C)cccc4Cl)s3)nc(C)n2)CC1] | (xx) |
| 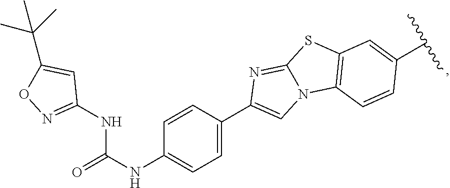 [SMILES: *c1ccc2c(c1)sc1nc(-c3ccc(NC(=O)Nc4cc(C(C)(C)C)on4)cc3)cn12] | (yy) |
| 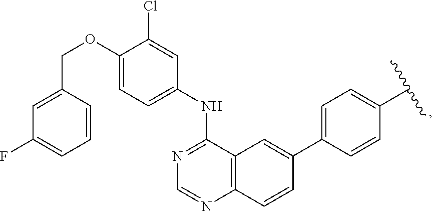 [SMILES: *c1ccc(-c2ccc3ncnc(Nc4ccc(OCc5cccc(F)c5)c(Cl)c4)c3c2)cc1] | (zz) |
| 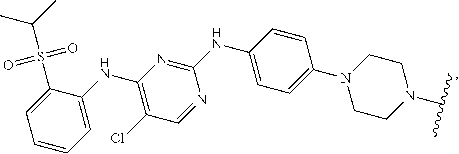 [SMILES: *N1CCN(c2ccc(Nc3ncc(Cl)c(Nc4ccccc4S(=O)(=O)C(C)C)n3)cc2)CC1] | (aaa) |
| 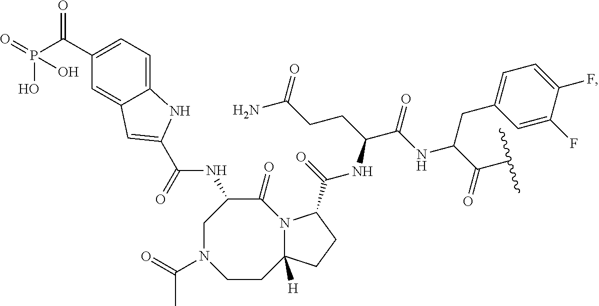 [SMILES: *C(=O)C(Cc1ccc(F)c(F)c1)NC(=O)[C@H](CCC(N)=O)NC(=O)[C@@H]1CC[C@@H]2CCN(C(C)=O)C[C@H](NC(=O)c3cc4cc(C(=O)P(=O)(O)O)ccc4[nH]3)C(=O)N21] | (bbb) |
| 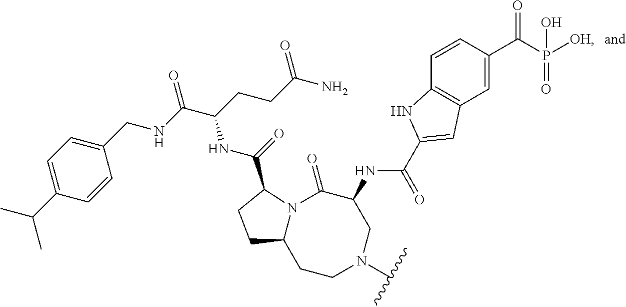 [SMILES: *N1CC[C@H]2CC[C@@H](C(=O)N[C@@H](CCC(N)=O)C(=O)NCc3ccc(C(C)C)cc3)N2C(=O)[C@@H](NC(=O)c2cc3cc(C(=O)P(=O)(O)O)ccc3[nH]2)C1 ; "and"] | (ccc) |
| 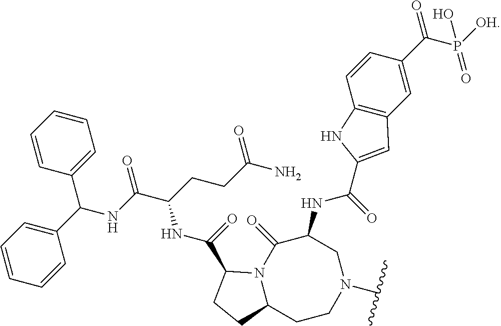 [SMILES: *N1CC[C@H]2CC[C@@H](C(=O)N[C@@H](CCC(N)=O)C(=O)NC(c3ccccc3)c3ccccc3)N2C(=O)[C@@H](NC(=O)c2cc3cc(C(=O)P(=O)(O)O)ccc3[nH]2)C1] | (ddd) |

TABLE B
Exemplified Linkers (L)

| | |
|---|---|
| 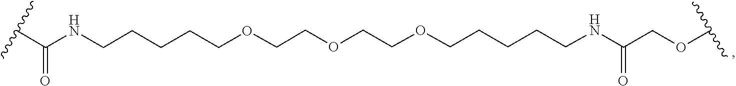 [SMILES: *OCC(=O)NCCCCCOCCOCCOCCCCCNC(*)=O] | (1) |
| 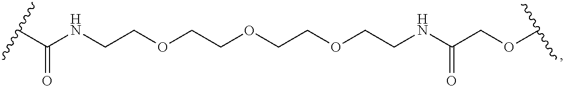 [SMILES: *OCC(=O)NCCOCCOCCOCCNC(*)=O] | (2) |
| 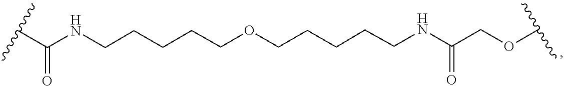 [SMILES: *OCC(=O)NCCCCCOCCCCCNC(*)=O] | (3) |
| 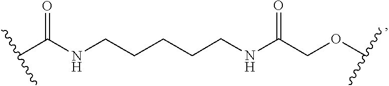 [SMILES: *OCC(=O)NCCCCCNC(*)=O] | (4) |
| 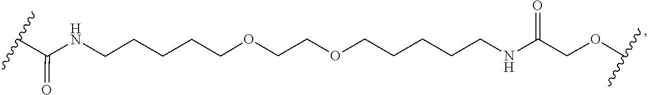 [SMILES: *OCC(=O)NCCCCCOCCOCCCCCNC(*)=O] | (5) |
| 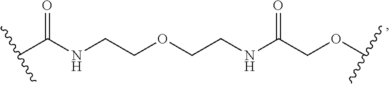 [SMILES: *OCC(=O)NCCOCCNC(*)=O] | (6) |
| 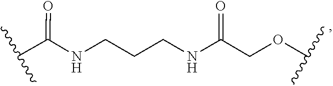 [SMILES: *OCC(=O)NCCCNC(*)=O] | (7) |
| 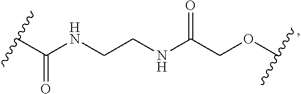 [SMILES: *OCC(=O)NCCNC(*)=O] | (8) |
| 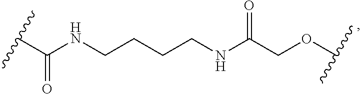 [SMILES: *OCC(=O)NCCCCNC(*)=O] | (9) |
| 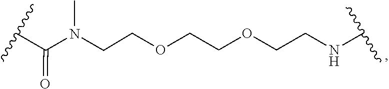 [SMILES: *NCCOCCOCCN(C)C(*)=O] | (10) |
| 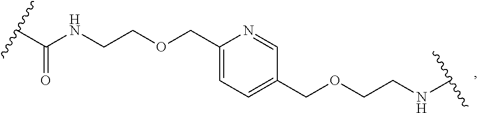 [SMILES: *NCCOCc1ccc(COCCNC(*)=O)nc1] | (11) |
| 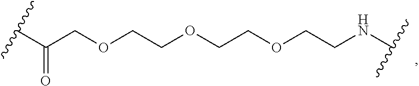 [SMILES: *NCCOCCOCCOCC(*)=O] | (12) |
| 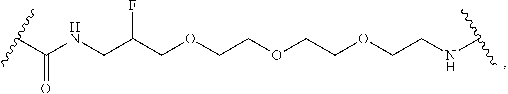 [SMILES: *NCCOCCOCCOCC(F)CNC(*)=O] | (13) |
| 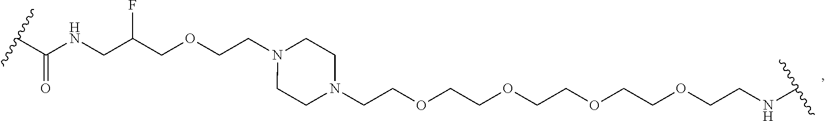 [SMILES: *NCCOCCOCCOCCOCCN1CCN(CCOCC(F)CNC(*)=O)CC1] | (14) |
| 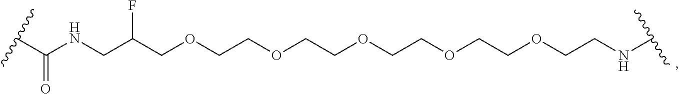 [SMILES: *NCCOCCOCCOCCOCCOCC(F)CNC(*)=O] | (15) |
| 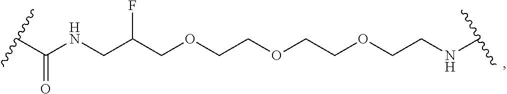 [SMILES: *NCCOCCOCCOCC(F)CNC(*)=O] | (16) |
| 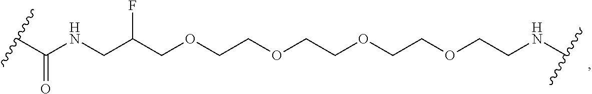 [SMILES: *NCCOCCOCCOCCOCC(F)CNC(*)=O] | (17) |
| 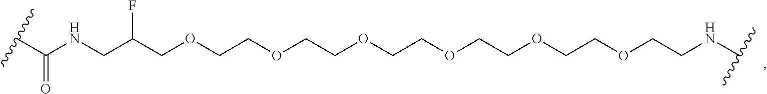 [SMILES: *NCCOCCOCCOCCOCCOCCOCC(F)CNC(*)=O] | (18) |
| 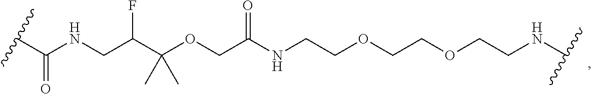 [SMILES: *NCCOCCOCCNC(=O)COC(C)(C)C(F)CNC(*)=O] | (19) |
| 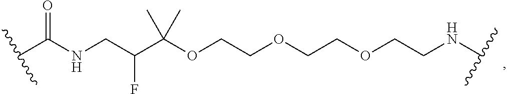 [SMILES: *NCCOCCOCCOC(C)(C)C(F)CNC(*)=O] | (20) |
| 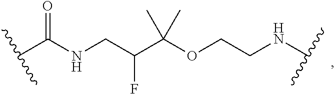 [SMILES: *NCCOC(C)(C)C(F)CNC(*)=O] | (21) |
| 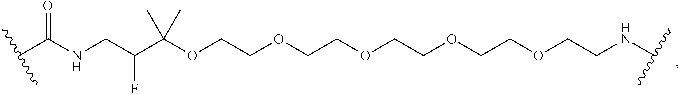 [SMILES: *NCCOCCOCCOCCOCCOC(C)(C)C(F)CNC(*)=O] | (22) |
| 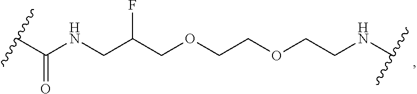 [SMILES: *NCCOCCOCC(F)CNC(*)=O] | (23) |
| 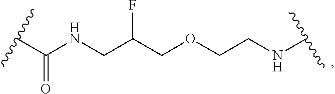 [SMILES: *NCCOCC(F)CNC(*)=O] | (24) |
| 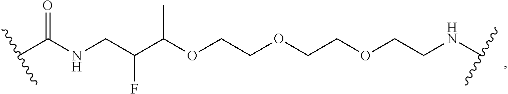 [SMILES: *NCCOCCOCCOC(C)C(F)CNC(*)=O] | (25) |
| 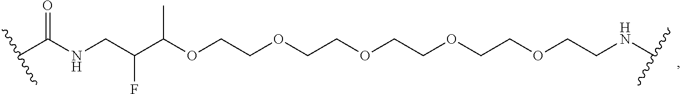 [SMILES: *NCCOCCOCCOCCOCCOC(C)C(F)CNC(*)=O] | (26) |
| 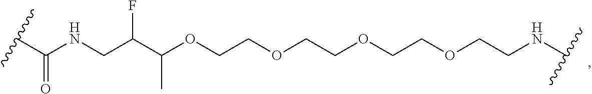 [SMILES: *NCCOCCOCCOCCOC(C)C(F)CNC(*)=O] | (27) |
| 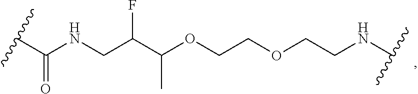 [SMILES: *NCCOCCOC(C)C(F)CNC(*)=O] | (28) |
| 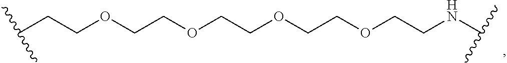 [SMILES: *CCOCCOCCOCCOCCN*] | (29) |
| 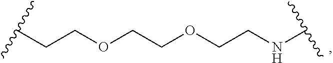 [SMILES: *CCOCCOCCN*] | (30) |
| 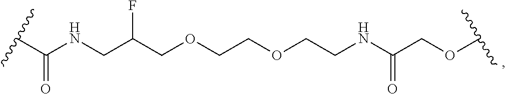 [SMILES: *OCC(=O)NCCOCCOCC(F)CNC(*)=O] | (31) |
| 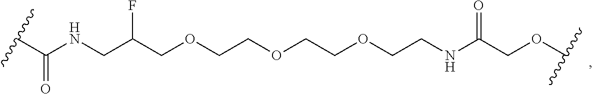 [SMILES: *OCC(=O)NCCOCCOCCOCC(F)CNC(*)=O] | (32) |
| 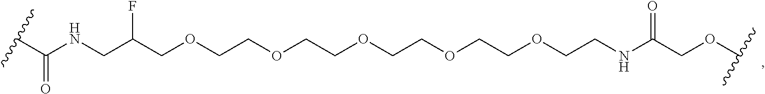 [SMILES: *OCC(=O)NCCOCCOCCOCCOCCOCC(F)CNC(*)=O] | (33) |
| 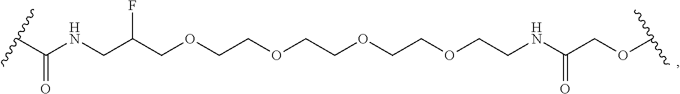 [SMILES: *OCC(=O)NCCOCCOCCOCCOCC(F)CNC(*)=O] | (34) |
| 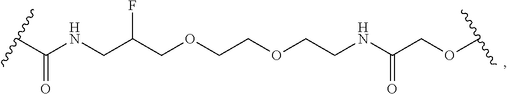 [SMILES: *OCC(=O)NCCOCCOCC(F)CNC(*)=O] | (35) |
| 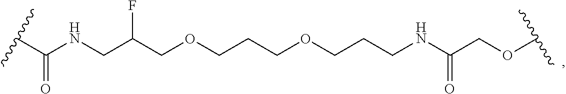 [SMILES: *OCC(=O)NCCCOCCCOCC(F)CNC(*)=O] | (36) |
| 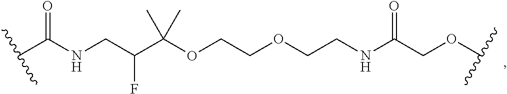 [SMILES: *OCC(=O)NCCOCCOC(C)(C)C(F)CNC(*)=O] | (37) |
| 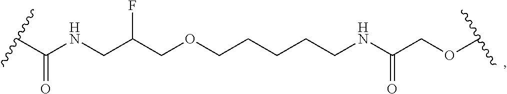 [SMILES: *OCC(=O)NCCCCCOCC(F)CNC(*)=O] | (38) |
| 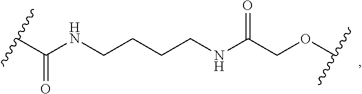 [SMILES: *OCC(=O)NCCCCNC(*)=O] | (39) |
| 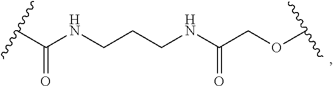 [SMILES: *OCC(=O)NCCCNC(*)=O] | (40) |
| 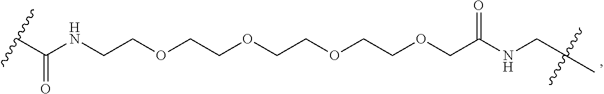 [SMILES: *CNC(=O)COCCOCCOCCOCCNC(*)=O] | (41) |
| 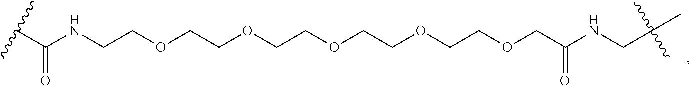 [SMILES: *CNC(=O)COCCOCCOCCOCCOCCNC(*)=O] | (42) |
| 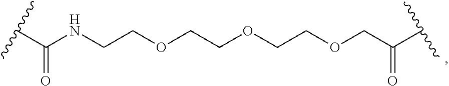 [SMILES: *C(=O)COCCOCCOCCNC(*)=O] | (43) |
| 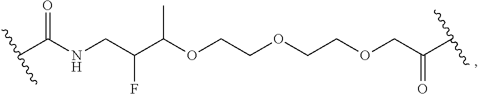 [SMILES: *C(=O)COCCOCCOC(C)C(F)CNC(*)=O] | (44) |
| 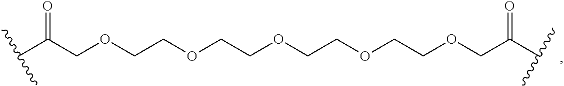 [SMILES: *C(=O)COCCOCCOCCOCCOCC(*)=O] | (45) |
| 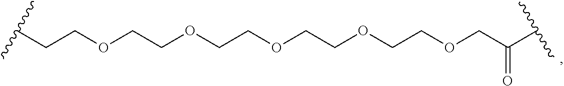 [SMILES: *CCOCCOCCOCCOCCOCC(*)=O] | (46) |
| 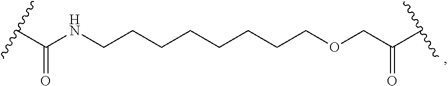 [SMILES: *C(=O)COCCCCCCCCNC(*)=O] | (47) |
| 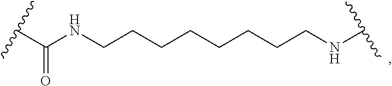 [SMILES: *NCCCCCCCCNC(*)=O] | (49) |
| 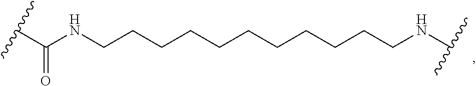 [SMILES: *NCCCCCCCCCCCNC(*)=O] | (50) |
| 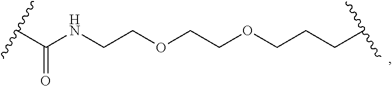 [SMILES: *CCCOCCOCCNC(*)=O] | (51) |
| 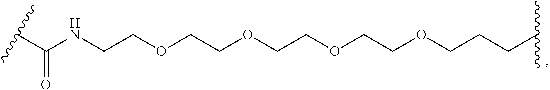 [SMILES: *CCCOCCOCCOCCOCCNC(*)=O] | (52) |
| 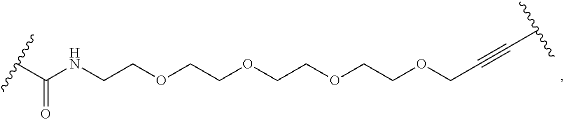 [SMILES: *C#CCOCCOCCOCCOCCNC(*)=O] | (53) |
| 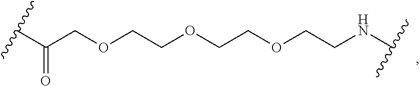 [SMILES: *NCCOCCOCCOCC(*)=O] | (54) |
| 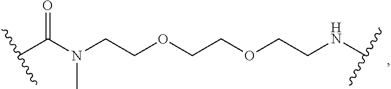 [SMILES: *NCCOCCOCCN(C)C(*)=O] | (55) |
| 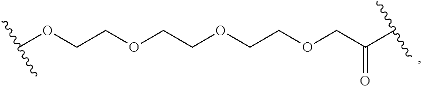 [SMILES: *OCCOCCOCCOCC(*)=O] | (56) |
| 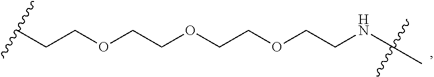 [SMILES: *CCOCCOCCOCCN*] | (57) |
| 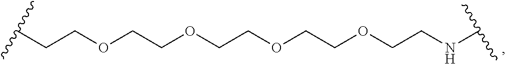 [SMILES: *CCOCCOCCOCCOCCN*] | (58) |
| 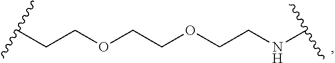 [SMILES: *CCOCCOCCN*] | (59) |
| 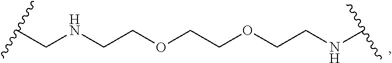 [SMILES: *CNCCOCCOCCN*] | (60) |
| 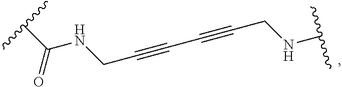 [SMILES: *NCC#CC#CCNC(*)=O] | (61) |
| 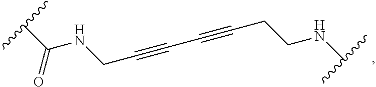 [SMILES: *NCCC#CC#CCNC(*)=O] | (62) |
| 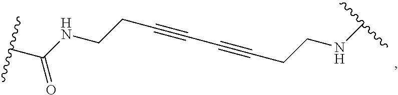 [SMILES: *NCCC#CC#CCCNC(*)=O] | (63) |
| 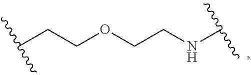 [SMILES: *CCOCCN*] | (64) |
| 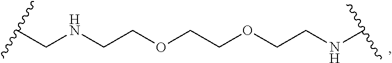 [SMILES: *CNCCOCCOCCN*] | (65) |
| 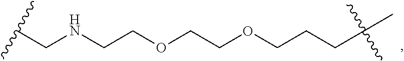 [SMILES: *CCCOCCOCCNC*] | (66) |
| 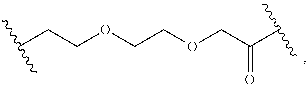 [SMILES: *CCOCCOCC(*)=O] | (67) |
| 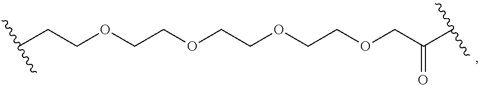 [SMILES: *CCOCCOCCOCCOCC(*)=O] | (68) |
| 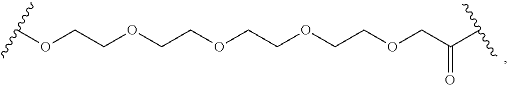 [SMILES: *OCCOCCOCCOCCOCC(*)=O] | (69) |
| 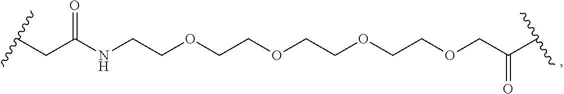 [SMILES: *CC(=O)NCCOCCOCCOCCOCC(*)=O] | (70) |
| 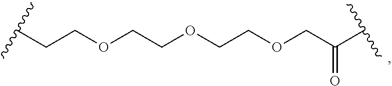 [SMILES: *CCOCCOCCOCC(*)=O] | (71) |
| 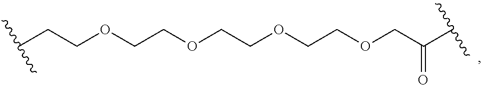 [SMILES: *CCOCCOCCOCCOCC(*)=O] | (72) |
| 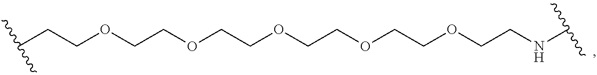 [SMILES: *CCOCCOCCOCCOCCOCCN*] | (73) |
| 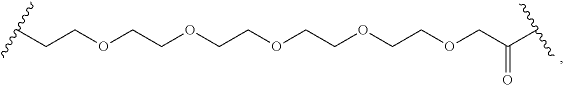 [SMILES: *CCOCCOCCOCCOCCOCC(*)=O] | (74) |
| 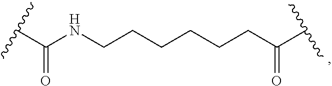 [SMILES: *C(=O)CCCCCCNC(*)=O] | (75) |
| 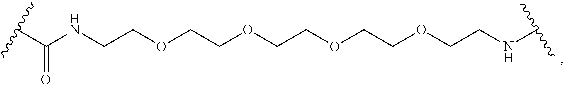 [SMILES: *NCCOCCOCCOCCOCCNC(*)=O] | (76) |
| 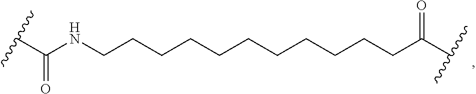 [SMILES: *C(=O)CCCCCCCCCCCNC(*)=O] | (77) |
| 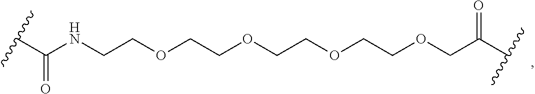 [SMILES: *C(=O)COCCOCCOCCOCCNC(*)=O] | (78) |
| 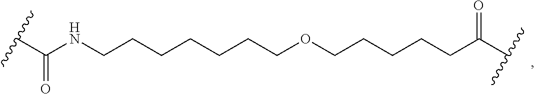 [SMILES: *C(=O)CCCCCOCCCCCCCNC(*)=O] | (79) |
| 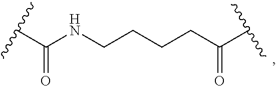 [SMILES: *C(=O)CCCCNC(*)=O] | (80) |
| 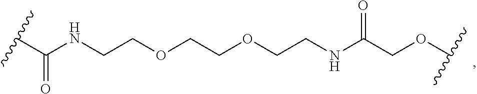 [SMILES: *OCC(=O)NCCOCCOCCNC(*)=O] | (81) |
| 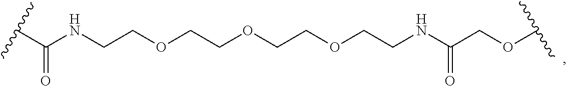 [SMILES: *OCC(=O)NCCOCCOCCOCCNC(*)=O] | (82) |
| 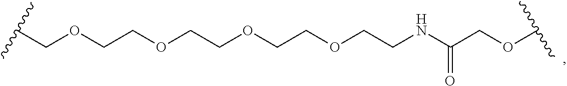 [SMILES: *COCCOCCOCCOCCNC(=O)CO*] | (83) |
| 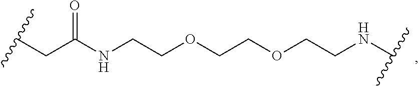 [SMILES: *CC(=O)NCCOCCOCCN*] | (84) |
| 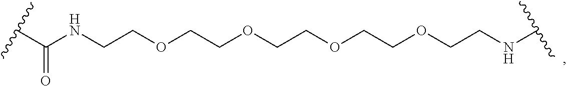 [SMILES: *NCCOCCOCCOCCOCCNC(*)=O] | (85) |
| 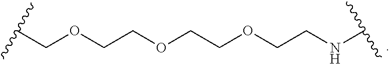 [SMILES: *COCCOCCOCCN*] | (86) |
| 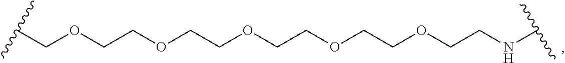 [SMILES: *COCCOCCOCCOCCOCCN*] | (87) |
| 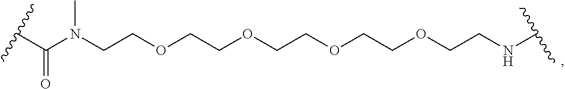 [SMILES: *NCCOCCOCCOCCOCCN(C)C(*)=O] | (88) |
| 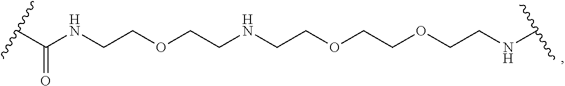 [SMILES: *NCCOCCOCCNCCOCCNC(*)=O] | (89) |
| 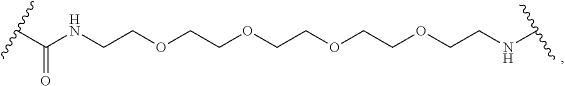 [SMILES: *NCCOCCOCCOCCOCCNC(*)=O] | (90) |
| 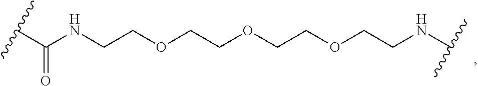 [SMILES: *NCCOCCOCCOCCNC(*)=O] | (91) |
| 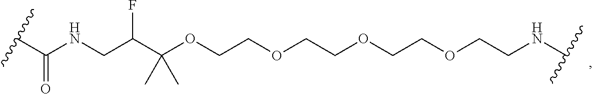 [SMILES: *NCCOCCOCCOCCOC(C)(C)C(F)CNC(*)=O] | (92) |
| 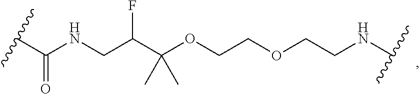 [SMILES: *NCCOCCOC(C)(C)C(F)CNC(*)=O] | (93) |
| 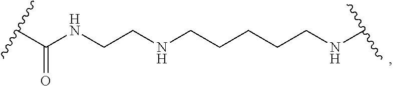 [SMILES: *NCCCCCNCCNC(*)=O] | (94) |
| 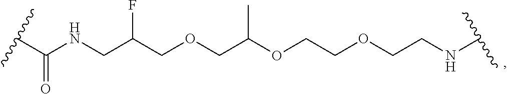 [SMILES: *NCCOCCOC(C)COCC(F)CNC(*)=O] | (95) |
| 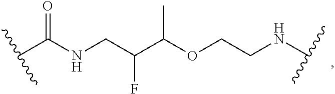 [SMILES: *NCCOC(C)C(F)CNC(*)=O] | (96) |
| 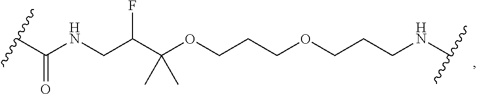 [SMILES: *NCCCOCCCOC(C)(C)C(F)CNC(*)=O] | (97) |
| 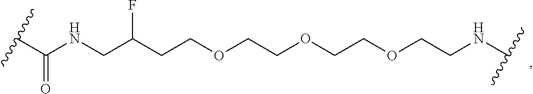 [SMILES: *NCCOCCOCCOCCC(F)CNC(*)=O] | (98) |
| 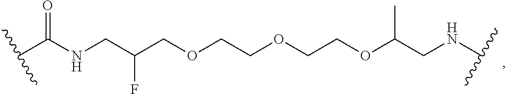 [SMILES: *NCC(C)OCCOCCOCC(F)CNC(*)=O] | (99) |
| 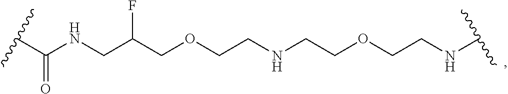 [SMILES: *NCCOCCNCCOCC(F)CNC(*)=O] | (100) |
| 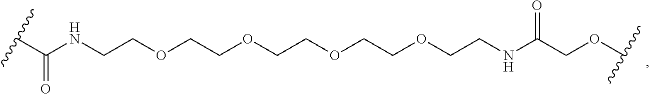 [SMILES: *OCC(=O)NCCOCCOCCOCCOCCNC(*)=O] | (101) |
| 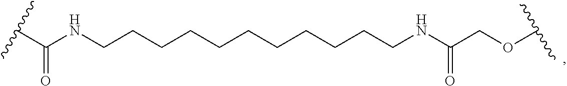 [SMILES: *OCC(=O)NCCCCCCCCCCCNC(*)=O] | (102) |
| 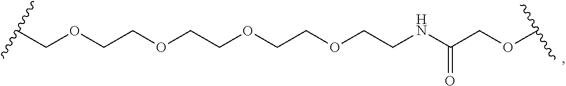 [SMILES: *COCCOCCOCCOCCNC(=O)CO*] | (103) |
| 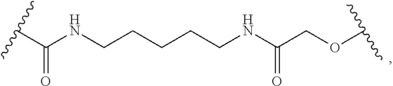 [SMILES: *OCC(=O)NCCCCCNC(*)=O] | (104) |
| 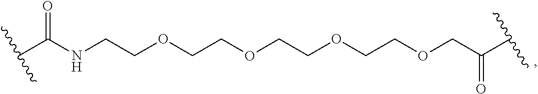 [SMILES: *C(=O)COCCOCCOCCOCCNC(*)=O] | (105) |
| 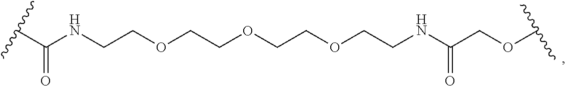 [SMILES: *OCC(=O)NCCOCCOCCOCCNC(*)=O] | (106) |
| 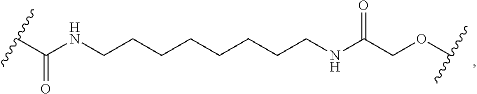 [SMILES: *OCC(=O)NCCCCCCCCNC(*)=O] | (107) |
| 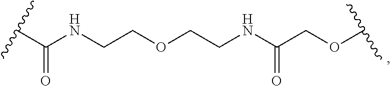 [SMILES: *OCC(=O)NCCOCCNC(*)=O] | (108) |
| 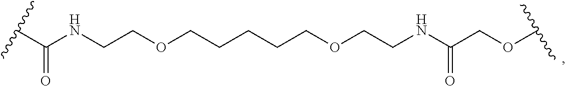 [SMILES: *OCC(=O)NCCOCCCCCOCCNC(*)=O] | (109) |
| 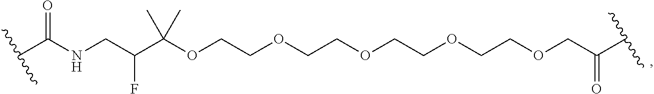 [SMILES: *C(=O)COCCOCCOCCOCCOC(C)(C)C(F)CNC(*)=O] | (110) |
| 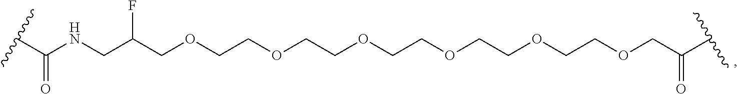 [SMILES: *C(=O)COCCOCCOCCOCCOCCOCC(F)CNC(*)=O] | (111) |
| 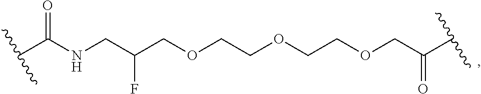 [SMILES: *C(=O)COCCOCCOCC(F)CNC(*)=O] | (112) |
| 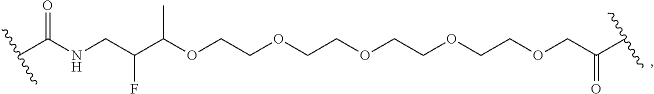 [SMILES: *C(=O)COCCOCCOCCOCCOC(C)C(F)CNC(*)=O] | (113) |
| 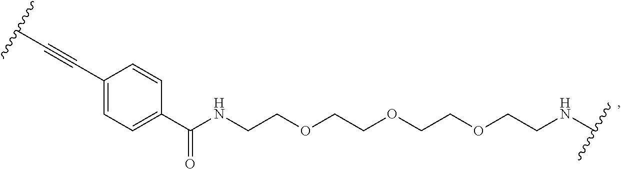 [SMILES: *C#Cc1ccc(C(=O)NCCOCCOCCOCCN*)cc1] | (114) |
| 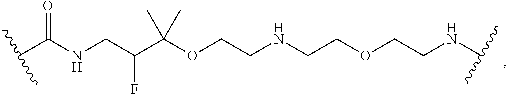 [SMILES: *NCCOCCNCCOC(C)(C)C(F)CNC(*)=O] | (115) |
| 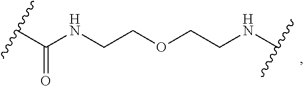 [SMILES: *NCCOCCNC(*)=O] | (116) |
| 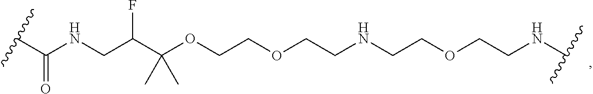 [SMILES: *NCCOCCNCCOCCOC(C)(C)C(F)CNC(*)=O] | (117) |
| 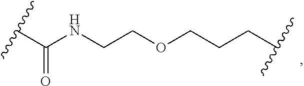 [SMILES: *CCCOCCNC(*)=O] | (118) |
| 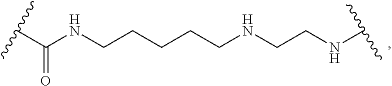 [SMILES: *NCCNCCCCCNC(*)=O] | |
| 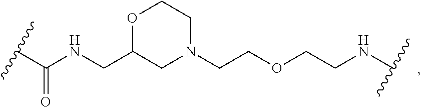 [SMILES: *NCCOCCN1CCOC(CNC(*)=O)C1] | (119) |
| 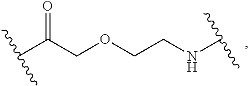 [SMILES: *NCCOCC(*)=O] | |
| 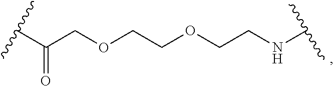 [SMILES: *NCCOCCOCC(*)=O] | (120) |
| 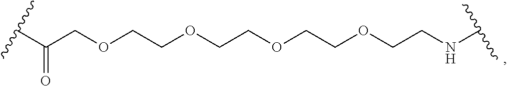 [SMILES: *NCCOCCOCCOCCOCC(*)=O] | (121) |
| 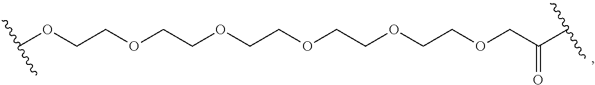 [SMILES: *OCCOCCOCCOCCOCCOCC(*)=O] | (122) |
| 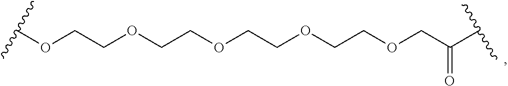 [SMILES: *OCCOCCOCCOCCOCC(*)=O] | (123) |
| 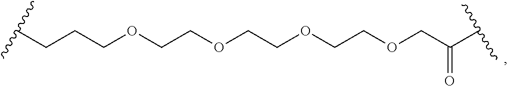 [SMILES: *CCCOCCOCCOCCOCC(*)=O] | (124) |
| 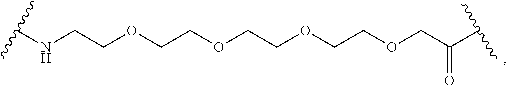 [SMILES: *NCCOCCOCCOCCOCC(*)=O] | (125) |
| 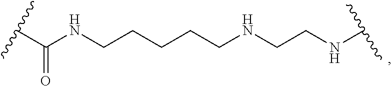 [SMILES: *NCCNCCCCCNC(*)=O] | (126) |
| 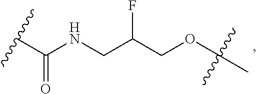 [SMILES: *OCC(F)CNC(*)=O] | |
| 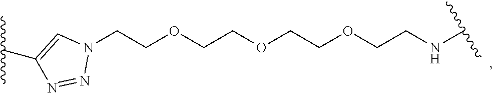 [SMILES: *NCCOCCOCCOCCn1cc(*)nn1] | (127) |
| 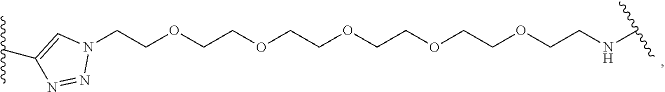 [SMILES: *NCCOCCOCCOCCOCCOCCn1cc(*)nn1] | (128) |
| 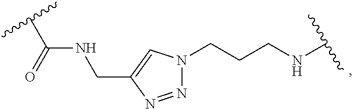 [SMILES: *NCCCn1cc(CNC(*)=O)nn1] | (129) |
| 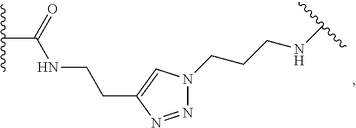 [SMILES: *NCCCn1cc(CCNC(*)=O)nn1] | (130) |
| 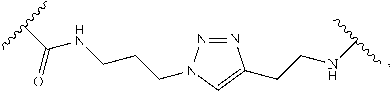 [SMILES: *NCCc1cn(CCCNC(*)=O)nn1] | (131) |
| 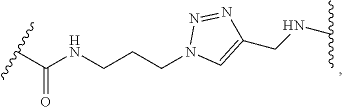 [SMILES: *NCc1cn(CCCNC(*)=O)nn1] | (132) |
| 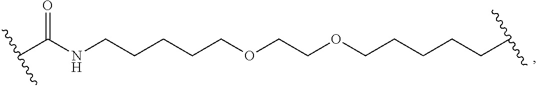 [SMILES: *CCCCCOCCOCCCCCNC(*)=O] | (133) |
| 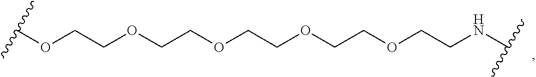 [SMILES: *NCCOCCOCCOCCOCCO*] | (134) |
| 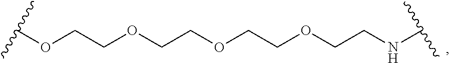 [SMILES: *NCCOCCOCCOCCO*] | (135) |
| 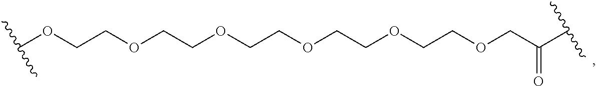 [SMILES: *OCCOCCOCCOCCOCCOCC(*)=O] | (136) |
| 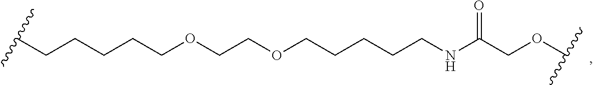 [SMILES: *CCCCCOCCOCCCCCNC(=O)CO*] | (137) |
| 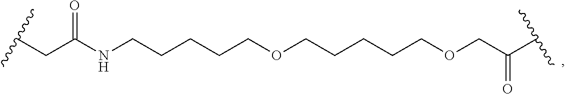 [SMILES: *CC(=O)NCCCCCOCCCCCOCC(*)=O] | (138) |
| 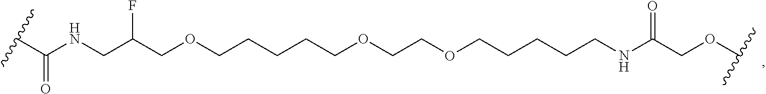 [SMILES: *OCC(=O)NCCCCCOCCOCCCCCOCC(F)CNC(*)=O] | (139) |
| 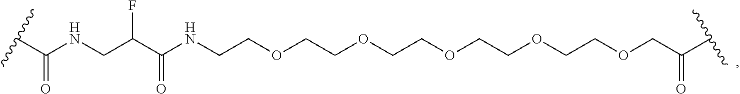 [SMILES: *C(=O)COCCOCCOCCOCCOCCNC(=O)C(F)CNC(*)=O] | (140) |
| 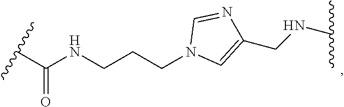 [SMILES: *NCc1cn(CCCNC(*)=O)cn1] | (141) |
| 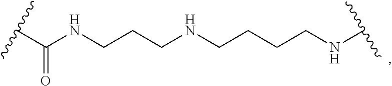 [SMILES: *NCCCCNCCCNC(*)=O] | (142) |
| 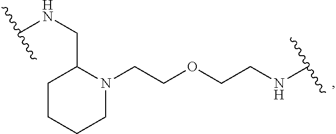 [SMILES: *NCCOCCN1CCCCC1CN*] | (143) |
| 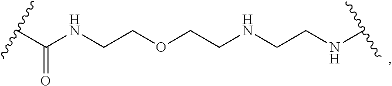 [SMILES: *NCCNCCOCCNC(*)=O] | (144) |
| 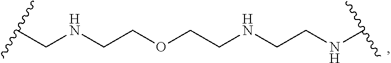 [SMILES: *CNCCOCCNCCN*] | (145) |
| 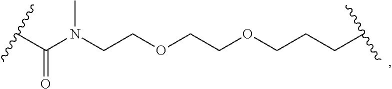 [SMILES: *CCCOCCOCCN(C)C(*)=O] | (146) |
| 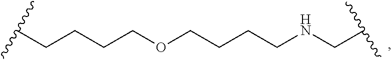 [SMILES: *CCCCOCCCCNC*] | (147) |
| 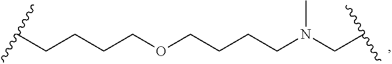 [SMILES: *CCCCOCCCCN(C)C*] | (148) |
| 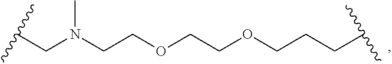 [SMILES: *CCCOCCOCCN(C)C*] | (149) |
| 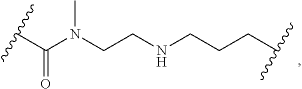 [SMILES: *CCCNCCN(C)C(*)=O] | (150) |
| 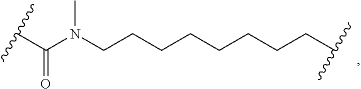 [SMILES: *CCCCCCCCN(C)C(*)=O] | (151) |
| 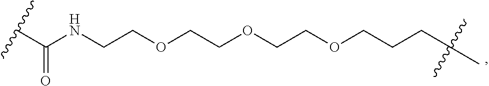 [SMILES: *CCCOCCOCCOCCNC(*)=O] | (152) |
| 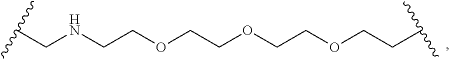 [SMILES: *CCOCCOCCOCCNC*] | (153) |
| 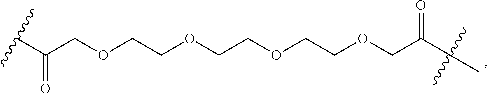 [SMILES: *C(=O)COCCOCCOCCOCC(*)=O] | (154) |
| 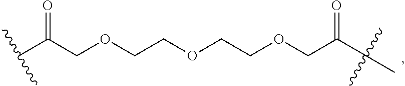 [SMILES: *C(=O)COCCOCCOCC(*)=O] | (155) |
| 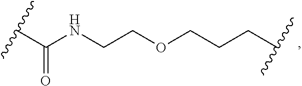 [SMILES: *CCCOCCNC(*)=O] | (156) |
| 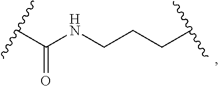 [SMILES: *CCCNC(*)=O] | (157) |
| 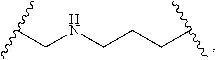 [SMILES: *CCCNC*] | (158) |
| 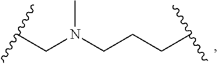 [SMILES: *CCCN(C)C*] | (159) |
| 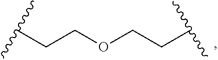 [SMILES: *CCOCC*] | (160) |
| 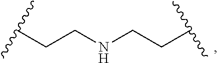 [SMILES: *CCNCC*] | (161) |
| 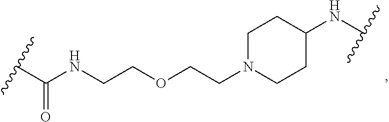 [SMILES: *NC1CCN(CCOCCNC(*)=O)CC1] | (162) |
| 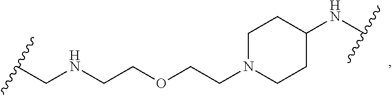 [SMILES: *CNCCOCCN1CCC(N*)CC1] | (163) |
| 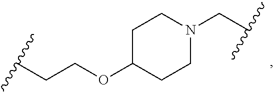 [SMILES: *CCOC1CCN(C*)CC1] | (164) |
| 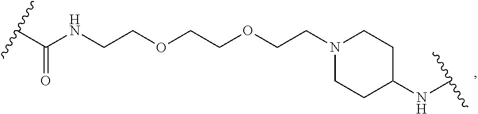 [SMILES: *NC1CCN(CCOCCOCCNC(*)=O)CC1] | (165) |
| 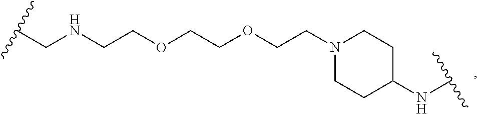 [SMILES: *CNCCOCCOCCN1CCC(N*)CC1] | (166) |
| 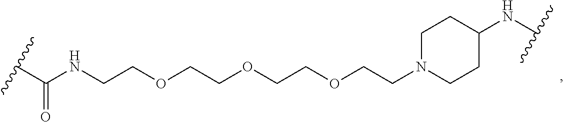 [SMILES: *NC1CCN(CCOCCOCCOCCNC(*)=O)CC1] | (167) |
| 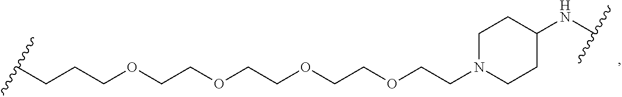 [SMILES: *CCCOCCOCCOCCOCCN1CCC(N*)CC1] | (168) |
| 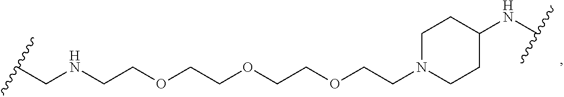 [SMILES: *CNCCOCCOCCOCCN1CCC(N*)CC1] | (169) |
| 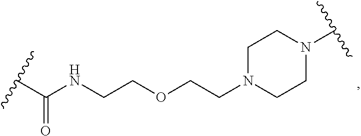 [SMILES: *C(=O)NCCOCCN1CCN(*)CC1] | (170) |
| 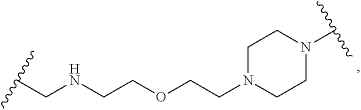 [SMILES: *CNCCOCCN1CCN(*)CC1] | (171) |
| 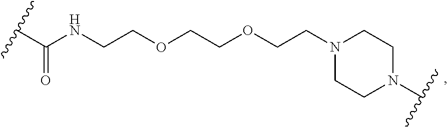 [SMILES: *C(=O)NCCOCCOCCN1CCN(*)CC1] | (172) |
| 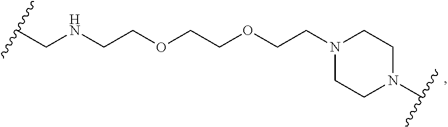 [SMILES: *CNCCOCCOCCN1CCN(*)CC1] | (173) |
| 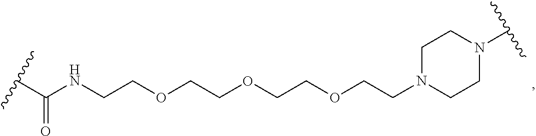 [SMILES: *C(=O)NCCOCCOCCOCCN1CCN(*)CC1] | (174) |
| 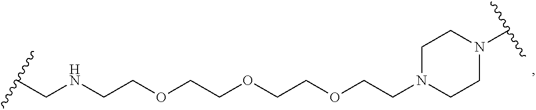 [SMILES: *CNCCOCCOCCOCCN1CCN(*)CC1] | (175) |
| 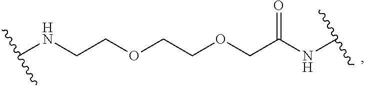 [SMILES: *NCCOCCOCC(=O)N*] | (176) |
| 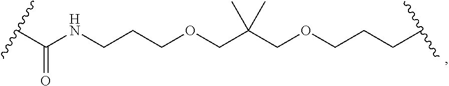 [SMILES: *CCCOCC(C)(C)COCCCNC(*)=O] | (177) |
| 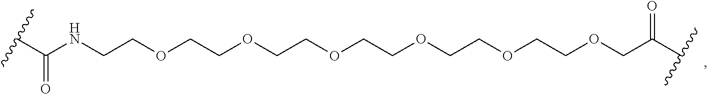 [SMILES: *C(=O)COCCOCCOCCOCCOCCOCCNC(*)=O] | (178) |
| 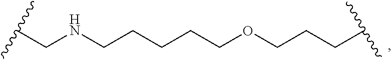 [SMILES: *CCCOCCCCCNC*] | (179) |
| 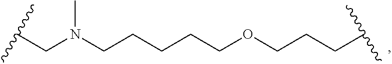 [SMILES: *CCCOCCCCCN(C)C*] | (180) |
| 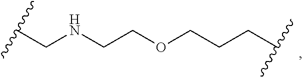 [SMILES: *CCCOCCNC*] | (181) |
| 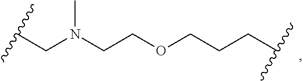 [SMILES: *CCCOCCN(C)C*] | (182) |
| 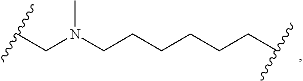 [SMILES: *CCCCCCN(C)C*] | (183) |
| 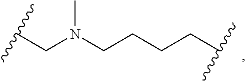 [SMILES: *CCCCN(C)C*] | (184) |
| 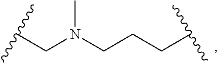 [SMILES: *CCCN(C)C*] | (185) |
| 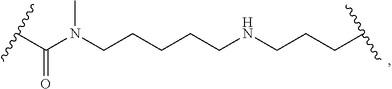 [SMILES: *CCCNCCCCCN(C)C(*)=O] | (186) |
| 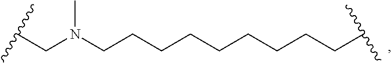 [SMILES: *CCCCCCCCCN(C)C*] | (187) |
| 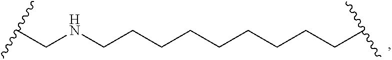 [SMILES: *CCCCCCCCCNC*] | (188) |
| 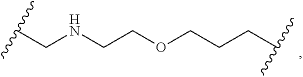 [SMILES: *CCCOCCNC*] | (189) |
| 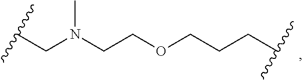 [SMILES: *CCCOCCN(C)C*] | (190) |
| 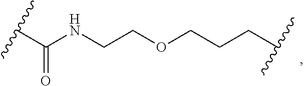 [SMILES: *CCCOCCNC(*)=O] | (191) |
| 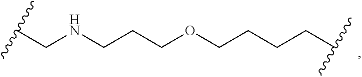 [SMILES: *CCCCOCCCNC*] | (192) |
| 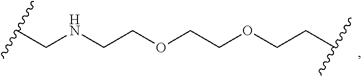 [SMILES: *CCOCCOCCNC*] | (193) |
| 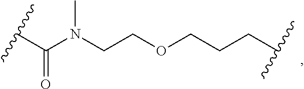 [SMILES: *CCCOCCN(C)C(*)=O] | (194) |
| 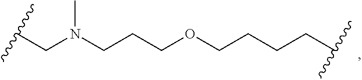 [SMILES: *CCCCOCCCN(C)C*] | (195) |
| 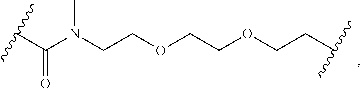 [SMILES: *CCOCCOCCN(C)C(*)=O] | (196) |
| 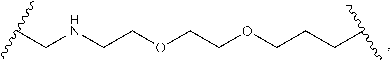 [SMILES: *CCCOCCOCCNC*] | (197) |
| 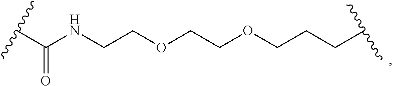 [SMILES: *CCCOCCOCCNC(*)=O] | (198) |
| 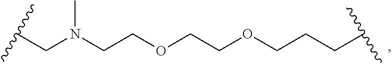 [SMILES: *CCCOCCOCCN(C)C*] | (199) |
| 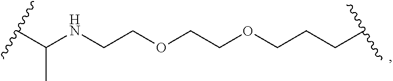 [SMILES: *CCCOCCOCCNC(*)C] | (200) |
| 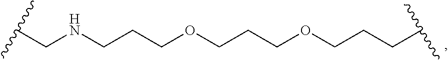 [SMILES: *CCCOCCCOCCCNC*] | (201) |
| 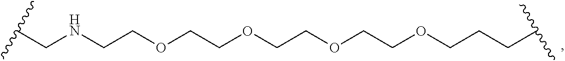 [SMILES: *CCCOCCOCCOCCOCCNC*] | (202) |
| 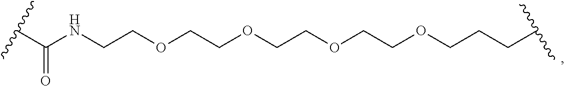 [SMILES: *CCCOCCOCCOCCOCCNC(*)=O] | (203) |
| 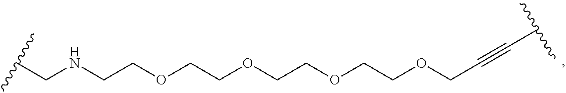 [SMILES: *C#CCOCCOCCOCCOCCNC*] | (204) |
| 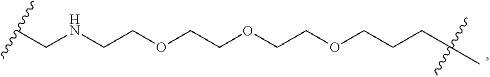 [SMILES: *CCCOCCOCCOCCNC*] | (205) |
| 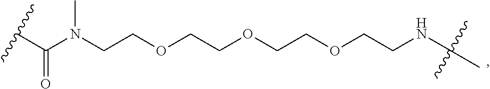 [SMILES: *NCCOCCOCCOCCN(C)C(*)=O] | (206) |
| 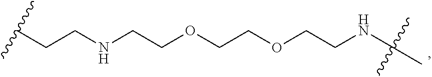 [SMILES: *CCNCCOCCOCCN*] | (207) |
| 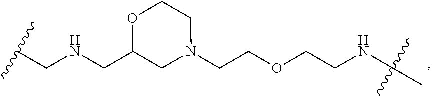 [SMILES: *CNCC1CN(CCOCCN*)CCO1] | (208) |
| 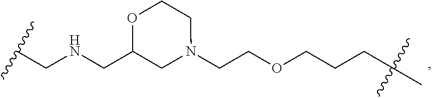 [SMILES: *CCCOCCN1CCOC(CNC*)C1] | (209) |
| 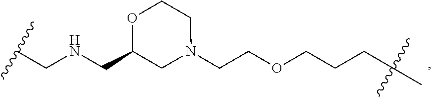 [SMILES: *CCCOCCN1CCO[C@H](CNC*)C1] | (210) |
| 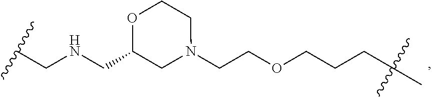 [SMILES: *CCCOCCN1CCO[C@@H](CNC*)C1] | (211) |
| 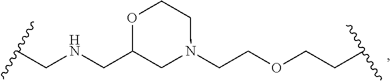 [SMILES: *CCOCCN1CCOC(CNC*)C1] | (212) |
| 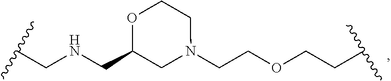 [SMILES: *CCOCCN1CCO[C@H](CNC*)C1] | (213) |
| 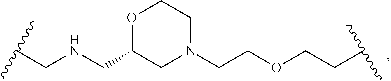 [SMILES: *CCOCCN1CCO[C@@H](CNC*)C1] | (214) |
| 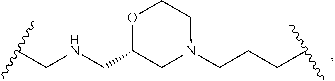 [SMILES: *CCCN1CCO[C@@H](CNC*)C1] | (215) |
| 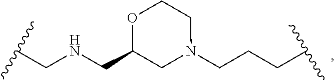 [SMILES: *CCCN1CCO[C@H](CNC*)C1] | (216) |
| 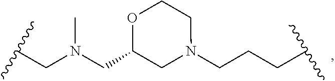 [SMILES: *CCCN1CCO[C@@H](CN(C)C*)C1] | (217) |
| 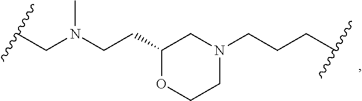 [SMILES: *CCCN1CCO[C@H](CCN(C)C*)C1] | (218) |
| 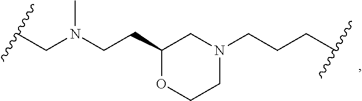 [SMILES: *CCCN1CCO[C@@H](CCN(C)C*)C1] | (219) |
| 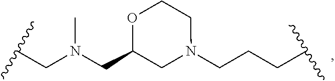 [SMILES: *CCCN1CCO[C@H](CN(C)C*)C1] | (220) |
| 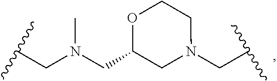 [SMILES: *CN(C)C[C@H]1CN(C*)CCO1] | (221) |
| 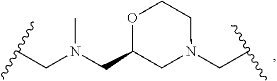 [SMILES: *CN(C)C[C@@H]1CN(C*)CCO1] | (222) |
| 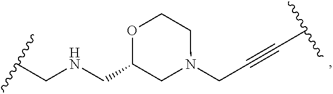 [SMILES: *C#CCN1CCO[C@@H](CNC*)C1] | (223) |
| 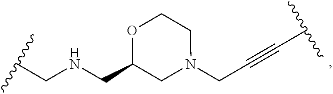 [SMILES: *C#CCN1CCO[C@H](CNC*)C1] | (224) |
| 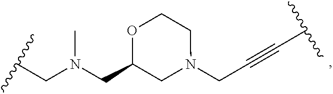 [SMILES: *C#CCN1CCO[C@H](CN(C)C*)C1] | (225) |
| 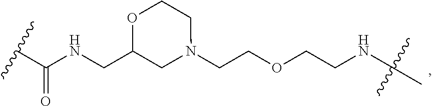 [SMILES: *NCCOCCN1CCOC(CNC(*)=O)C1] | (226) |
| 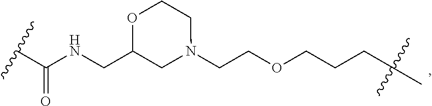 [SMILES: *CCCOCCN1CCOC(CNC(*)=O)C1] | (227) |
| 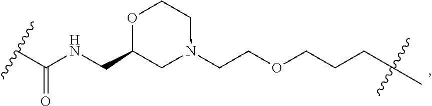 [SMILES: *CCCOCCN1CCO[C@H](CNC(*)=O)C1] | (228) |
| 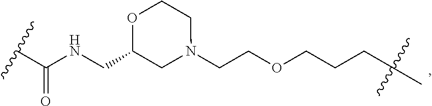 [SMILES: *CCCOCCN1CCO[C@@H](CNC(*)=O)C1] | (229) |
| 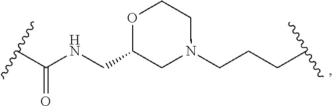 [SMILES: *CCCN1CCO[C@@H](CNC(*)=O)C1] | (230) |
| 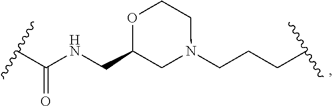 [SMILES: *CCCN1CCO[C@H](CNC(*)=O)C1] | (231) |
| 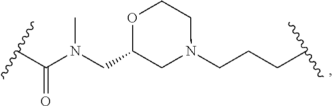 [SMILES: *CCCN1CCO[C@@H](CN(C)C(*)=O)C1] | (232) |
| 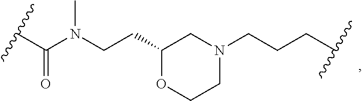 [SMILES: *CCCN1CCO[C@H](CCN(C)C(*)=O)C1] | (233) |
| 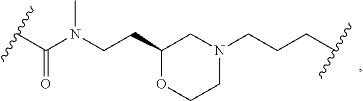 [SMILES: *CCCN1CCO[C@@H](CCN(C)C(*)=O)C1] | (234) |
| 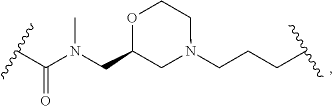 [SMILES: *CCCN1CCO[C@H](CN(C)C(*)=O)C1] | (235) |
| 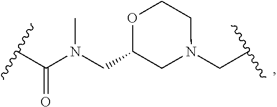 [SMILES: *CN1CCO[C@@H](CN(C)C(*)=O)C1] | (236) |
| 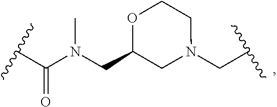 [SMILES: *CN1CCO[C@H](CN(C)C(*)=O)C1] | (237) |
| 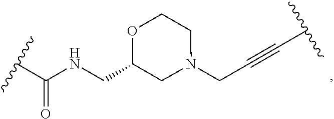 [SMILES: *C#CCN1CCO[C@@H](CNC(*)=O)C1] | (238) |
| 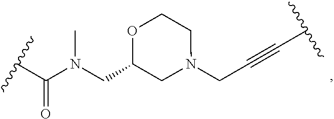 [SMILES: *C#CCN1CCO[C@@H](CN(C)C(*)=O)C1] | (239) |
| 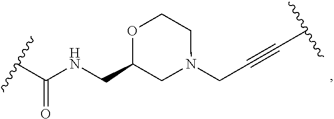 [SMILES: *C#CCN1CCO[C@H](CNC(*)=O)C1] | (240) |
| 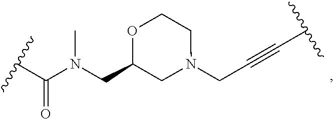 [SMILES: *C#CCN1CCO[C@H](CN(C)C(*)=O)C1] | (241) |
| 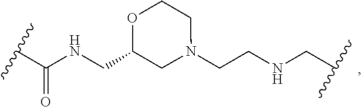 [SMILES: *CNCCN1CCO[C@@H](CNC(*)=O)C1] | (242) |
| 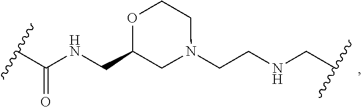 [SMILES: *CNCCN1CCO[C@H](CNC(*)=O)C1] | (243) |
| 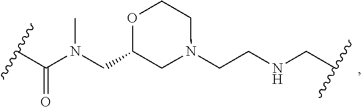 [SMILES: *CNCCN1CCO[C@@H](CN(C)C(*)=O)C1] | (244) |
| 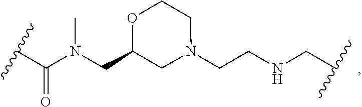 [SMILES: *CNCCN1CCO[C@H](CN(C)C(*)=O)C1] | (245) |
| 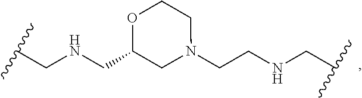 [SMILES: *CNCCN1CCO[C@@H](CNC*)C1] | (246) |
| 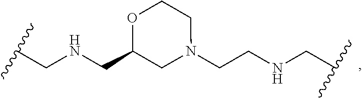 [SMILES: *CNCCN1CCO[C@H](CNC*)C1] | (247) |
| 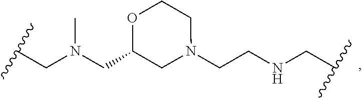 [SMILES: *CNCCN1CCO[C@@H](CN(C)C*)C1] | (248) |
| 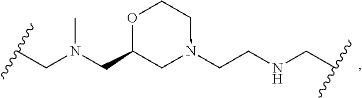 [SMILES: *CNCCN1CCO[C@H](CN(C)C*)C1] | (249) |
| 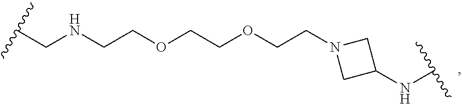 [SMILES: *CNCCOCCOCCN1CC(N*)C1] | (250) |
| 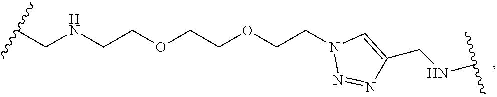 [SMILES: *CNCCOCCOCCn1cc(CN*)nn1] | (251) |
| 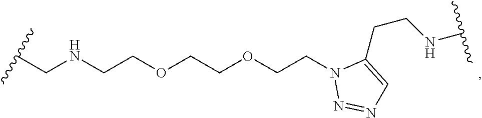 [SMILES: *CNCCOCCOCCn1nncc1CCN*] | (253) |
| 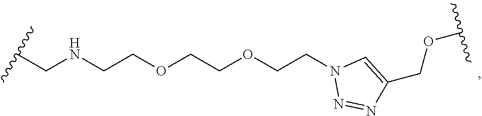 [SMILES: *CNCCOCCOCCn1cc(CO*)nn1] | (254) |
| 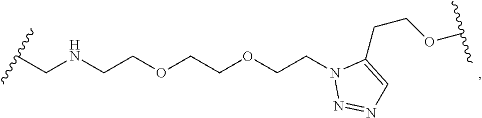 [SMILES: *CNCCOCCOCCn1nncc1CCO*] | (255) |
| 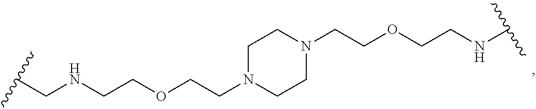 [SMILES: *CNCCOCCN1CCN(CCOCCN*)CC1] | (256) |
| 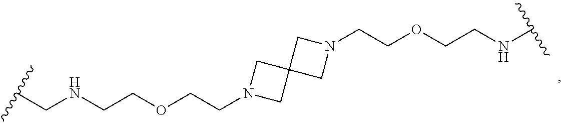 [SMILES: *CNCCOCCN1CC2(CN(CCOCCN*)C2)C1] | (257) |
| 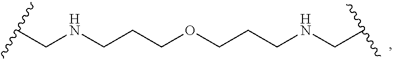 [SMILES: *CNCCCOCCCNC*] | (258) |
| 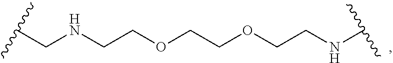 [SMILES: *CNCCOCCOCCN*] | (259) |
| 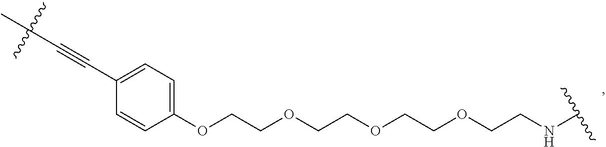 [SMILES: *C#Cc1ccc(OCCOCCOCCOCCN*)cc1] | (260) |
| 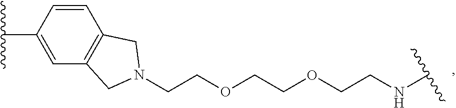 [SMILES: *NCCOCCOCCN1Cc2ccc(*)cc2C1] | (261) |
| 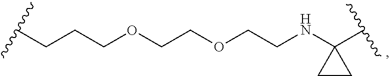 [SMILES: *CCCOCCOCCNC1(*)CC1] | (262) |
| 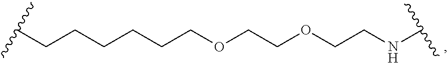 [SMILES: *CCCCCCOCCOCCN*] | (263) |
| 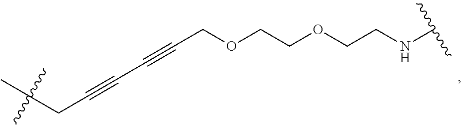 [SMILES: *CC#CC#CCOCCOCCN*] | (264) |
| 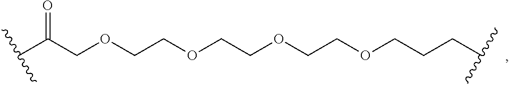 [SMILES: *CCCOCCOCCOCCOCC(*)=O] | (265) |
| 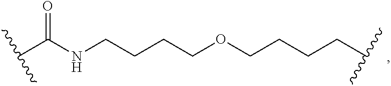 [SMILES: *CCCCOCCCCNC(*)=O] | (266) |
| 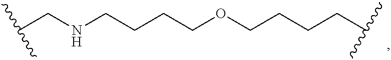 [SMILES: *CCCCOCCCCNC*] | (267) |
| 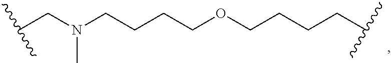 [SMILES: *CCCCOCCCCN(C)C*] | (268) |
| 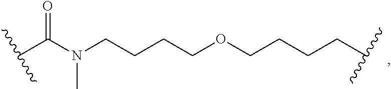 [SMILES: *CCCCOCCCCN(C)C(*)=O] | (269) |
| 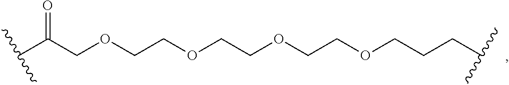 [SMILES: *CCCOCCOCCOCCOCC(*)=O] | (270) |
| 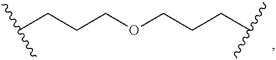 [SMILES: *CCCOCCC*] | (271) |
| 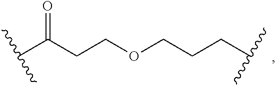 [SMILES: *CCCOCCC(*)=O] | (272) |
| 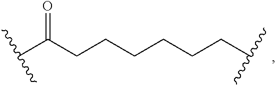 [SMILES: *CCCCCCC(*)=O] | (273) |
| 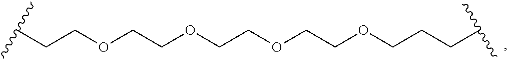 [SMILES: *CCCOCCOCCOCCOCC*] | (274) |
| 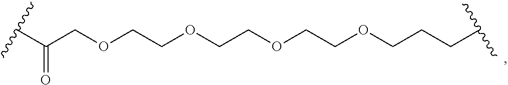 [SMILES: *CCCOCCOCCOCCOCC(*)=O] | (275) |
| 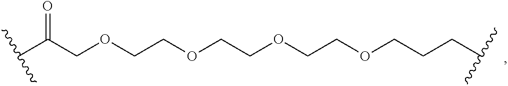 [SMILES: *CCCOCCOCCOCCOCC(*)=O] | (276) |
| 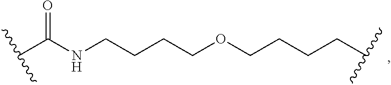 [SMILES: *CCCCOCCCCNC(*)=O] | (277) |
| 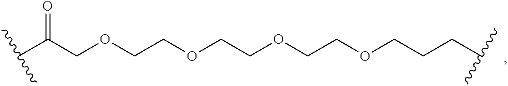 [SMILES: *CCCOCCOCCOCCOCC(*)=O] | (278) |
| 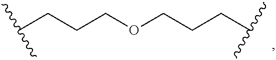 [SMILES: *CCCOCCC*] | (279) |
| 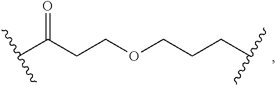 [SMILES: *CCCOCCC(*)=O] | (280) |
| 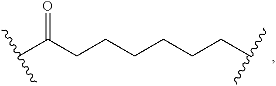 [SMILES: *CCCCCCC(*)=O] | (281) |
| 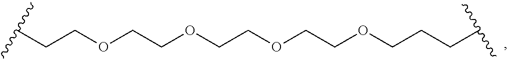 [SMILES: *CCCOCCOCCOCCOCC*] | (282) |
| 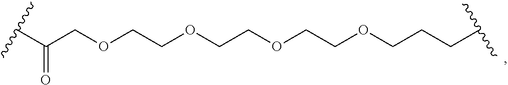 [SMILES: *CCCOCCOCCOCCOCC(*)=O] | (283) |
| 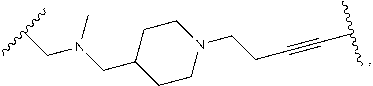 [SMILES: *C#CCCN1CCC(CN(C)C*)CC1] | (284) |
| 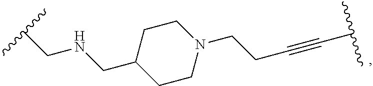 [SMILES: *C#CCCN1CCC(CNC*)CC1] | (285) |
| 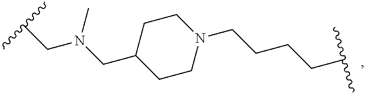 [SMILES: *CCCCN1CCC(CN(C)C*)CC1] | (286) |
| 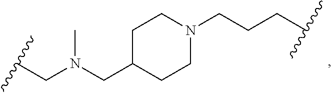 [SMILES: *CCCN1CCC(CN(C)C*)CC1] | (287) |
| 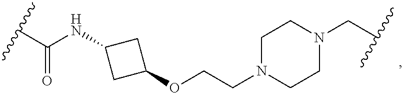 [SMILES: *CN1CCN(CCO[C@H]2C[C@H](NC(*)=O)C2)CC1] | (288) |
| 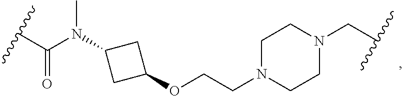 [SMILES: *CN1CCN(CCO[C@H]2C[C@H](N(C)C(*)=O)C2)CC1] | (289) |
| 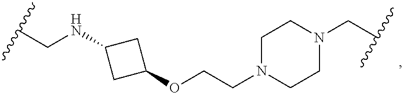 [SMILES: *CN[C@H]1C[C@H](OCCN2CCN(C*)CC2)C1] | (290) |
| 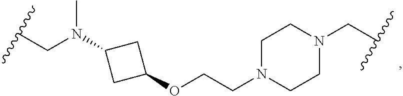 [SMILES: *CN1CCN(CCO[C@H]2C[C@H](N(C)C*)C2)CC1] | (291) |
| 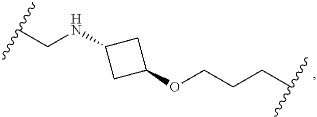 [SMILES: *CCCO[C@H]1C[C@H](NC*)C1] | (292) |
| 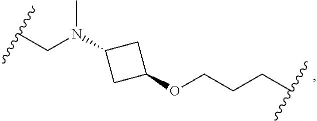 [SMILES: *CCCO[C@H]1C[C@H](N(C)C*)C1] | (293) |
| 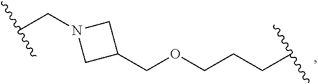 [SMILES: *CCCOCC1CN(C*)C1] | (294) |
| 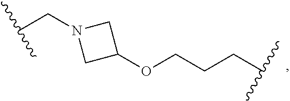 [SMILES: *CCCOC1CN(C*)C1] | (295) |
| 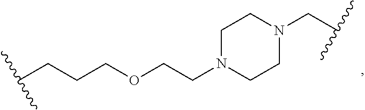 [SMILES: *CCCOCCN1CCN(C*)CC1] | (296) |
| 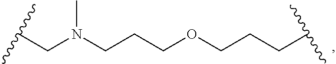 [SMILES: *CCCOCCCN(C)C*] | (297) |
| 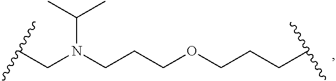 [SMILES: *CCCOCCCN(C*)C(C)C] | (298) |
| 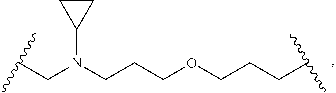 [SMILES: *CCCOCCCN(C*)C1CC1] | (299) |
| 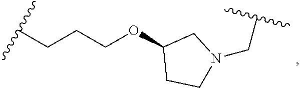 [SMILES: *CCCO[C@@H]1CCN(C*)C1] | (300) |
| 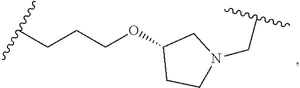 [SMILES: *CCCO[C@H]1CCN(C*)C1] | (301) |
| 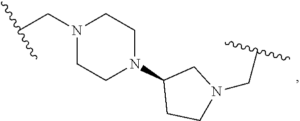 [SMILES: *CN1CCN([C@@H]2CCN(C*)C2)CC1] | (302) |
| 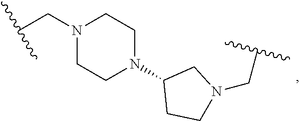 [SMILES: *CN1CCN([C@H]2CCN(C*)C2)CC1] | (303) |
| 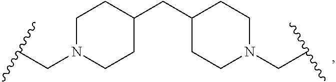 [SMILES: *CN1CCC(CC2CCN(C*)CC2)CC1] | (304) |
| 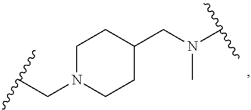 [SMILES: *CN1CCC(CN(*)C)CC1] | (305) |
| 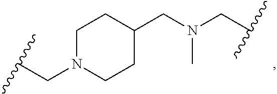 [SMILES: *CN(C)CC1CCN(C*)CC1] | (306) |
| 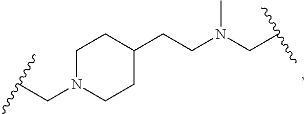 [SMILES: *CN(C)CCC1CCN(C*)CC1] | (307) |
| 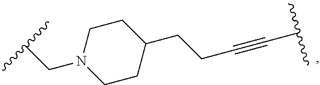 [SMILES: *C#CCCC1CCN(C*)CC1] | (308) |
| 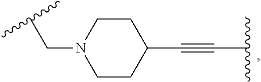 [SMILES: *C#CC1CCN(C*)CC1] | (309) |
| 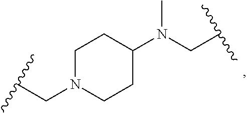 [SMILES: *CN1CCC(N(C)C*)CC1] | (310) |
| 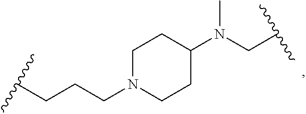 [SMILES: *CCCN1CCC(N(C)C*)CC1] | (311) |
| 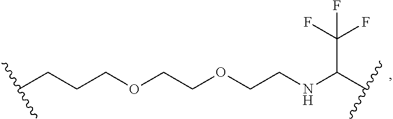 [SMILES: *CCCOCCOCCNC(*)C(F)(F)F] | (312) |
| 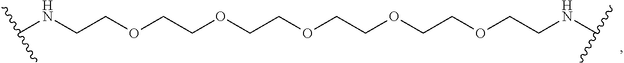 [SMILES: *NCCOCCOCCOCCOCCOCCN*] | (313) |
| 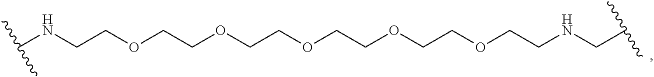 [SMILES: *CNCCOCCOCCOCCOCCOCCN*] | (314) |
| 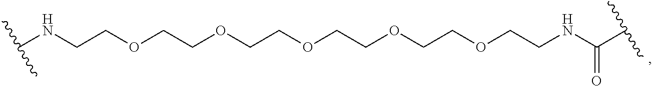 [SMILES: *NCCOCCOCCOCCOCCOCCNC(*)=O] | (315) |
| 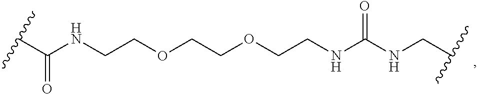 [SMILES: *CNC(=O)NCCOCCOCCNC(*)=O] | (316) |
| 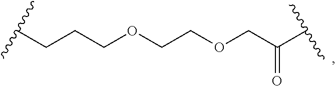 [SMILES: *CCCOCCOCC(*)=O] | (317) |
| 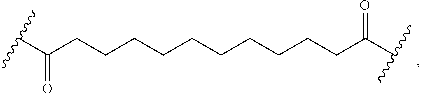 [SMILES: *C(=O)CCCCCCCCCCC(*)=O] | (318) |
| 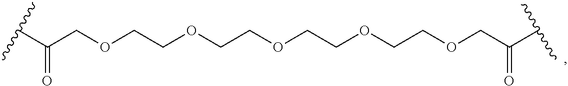 [SMILES: *C(=O)COCCOCCOCCOCCOCC(*)=O] | (319) |
| 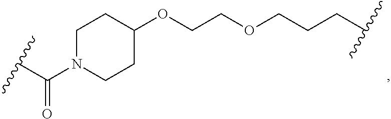 [SMILES: *CCCOCCOC1CCN(C(*)=O)CC1] | (320) |
| 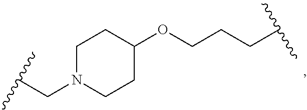 [SMILES: *CCCOC1CCN(C*)CC1] | (321) |
| 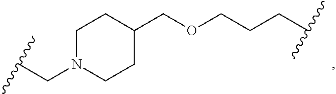 [SMILES: *CCCOCC1CCN(C*)CC1] | (322) |
| 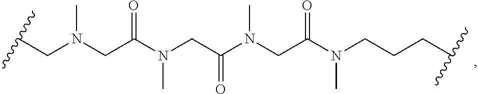 [SMILES: *CCCN(C)C(=O)CN(C)C(=O)CN(C)C(=O)CN(C)C*] | (323) |
| 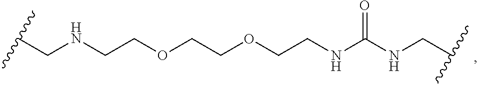 [SMILES: *CNCCOCCOCCNC(=O)NC*] | (324) |
| 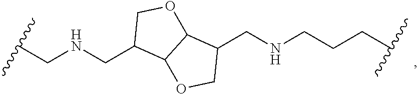 [SMILES: *CCCNCC1COC2C(CNC*)COC12] | (325) |
| 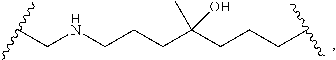 [SMILES: *CCCC(C)(O)CCCNC*] | (326) |
| 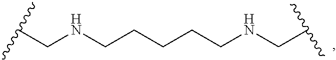 [SMILES: *CNCCCCCNC*] | (327) |
| 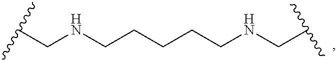 [SMILES: *CNCCCCCNC*] | (328) |
| 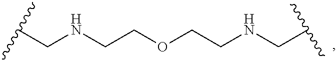 [SMILES: *CNCCOCCNC*] | (329) |
| 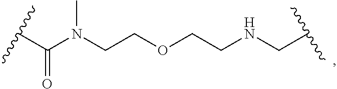 [SMILES: *CNCCOCCN(C)C(*)=O] | (330) |
| 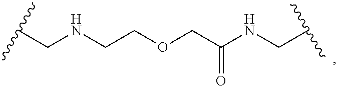 [SMILES: *CNCCOCC(=O)NC*] | (331) |
| 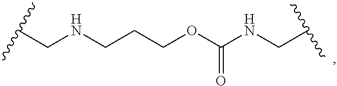 [SMILES: *CNCCCOC(=O)NC*] | (332) |
| 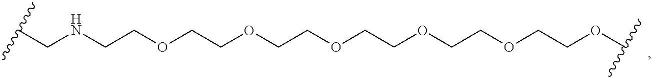 [SMILES: *CNCCOCCOCCOCCOCCOCCO*] | (333) |
| 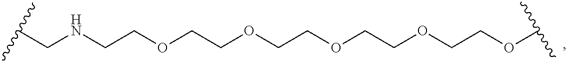 [SMILES: *CNCCOCCOCCOCCOCCO*] | (334) |
| 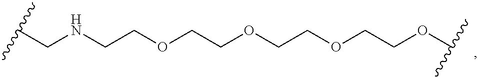 [SMILES: *CNCCOCCOCCOCCO*] | (335) |
| 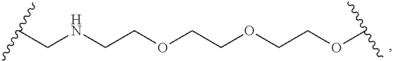 [SMILES: *CNCCOCCOCCO*] | (336) |
| | (337) |
| 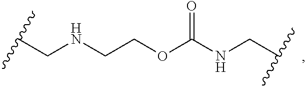 [SMILES: *CNCCOC(=O)NC*] | (338) |
| 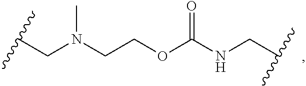 [SMILES: *CNC(=O)OCCN(C)C*] | (339) |
| 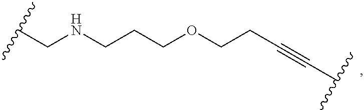 [SMILES: *C#CCCOCCCNC*] | (340) |
| 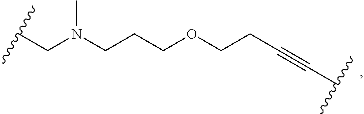 [SMILES: *C#CCCOCCCN(C)C*] | (341) |
| 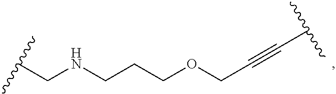 [SMILES: *C#CCOCCCNC*] | (342) |
| 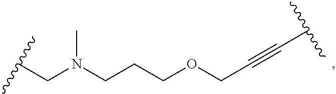 [SMILES: *C#CCOCCCN(C)C*] | (343) |
| 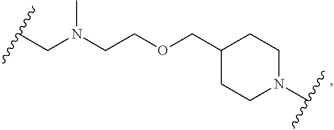 [SMILES: *CN(C)CCOCC1CCN(*)CC1] | (344) |
| 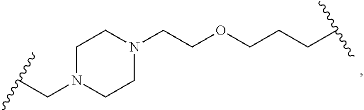 [SMILES: *CCCOCCN1CCN(C*)CC1] | (345) |
| 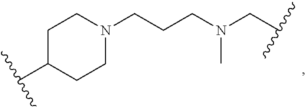 [SMILES: *CN(C)CCCN1CCC(*)CC1] | (346) |
| 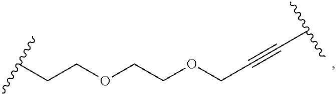 [SMILES: *C#CCOCCOCC*] | (347) |
| 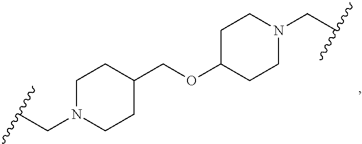 [SMILES: *CN1CCC(COC2CCN(C*)CC2)CC1] | (348) |
| 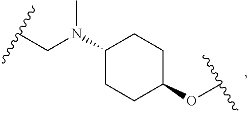 [SMILES: *CN(C)[C@H]1CC[C@H](O*)CC1] | (349) |
| 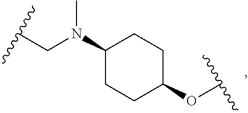 [SMILES: *CN(C)[C@H]1CC[C@@H](O*)CC1] | (350) |
| 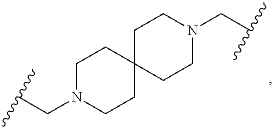 [SMILES: *CN1CCC2(CC1)CCN(C*)CC2] | (351) |
| 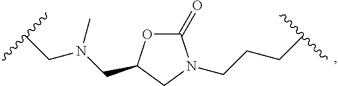 [SMILES: *CCCN1C[C@@H](CN(C)C*)OC1=O] | (352) |
| 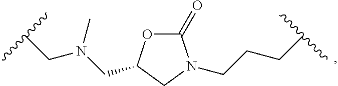 [SMILES: *CCCN1C[C@H](CN(C)C*)OC1=O] | (353) |
| 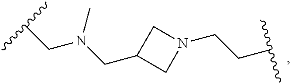 [SMILES: *CCN1CC(CN(C)C*)C1] | (354) |
| 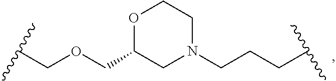 [SMILES: *CCCN1CCO[C@@H](COC*)C1] | (355) |
| 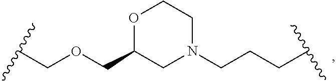 [SMILES: *CCCN1CCO[C@H](COC*)C1] | (356) |
| 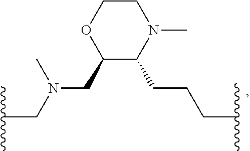 [SMILES: *CCC[C@@H]1[C@@H](CN(C)C*)OCCN1C] | (357) |
| 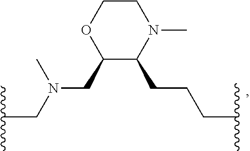 [SMILES: *CCC[C@H]1[C@@H](CN(C)C*)OCCN1C] | (358) |
| 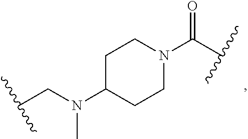 [SMILES: *CN(C)C1CCN(C(*)=O)CC1] | (359) |
| 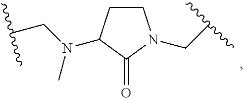 [SMILES: *CN1CCC(N(C)C*)C1=O] | (360) |
| 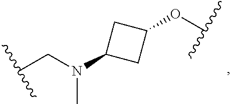 [SMILES: *CN(C)[C@H]1C[C@H](O*)C1] | (361) |
| 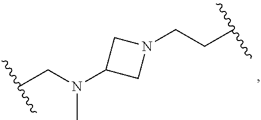 [SMILES: *CCN1CC(N(C)C*)C1] | (362) |
| 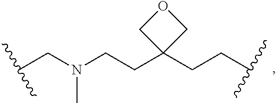 [SMILES: *CCC1(CCN(C)C*)COC1] | (363) |
| 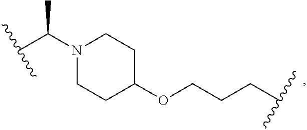 [SMILES: *CCCOC1CCN([C@@H](*)C)CC1] | (364) |
| 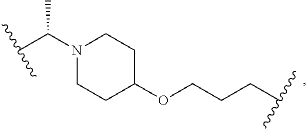 [SMILES: *CCCOC1CCN([C@H](*)C)CC1] | (365) |
| 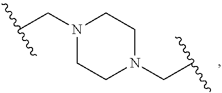 [SMILES: *CN1CCN(C*)CC1] | (366) |
| 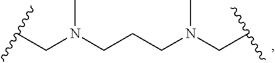 [SMILES: *CN(C)CCCN(C)C*] | (367) |
| 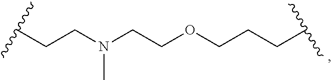 [SMILES: *CCCOCCN(C)CC*] | (368) |
| 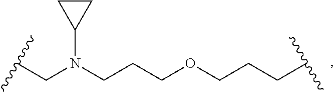 [SMILES: *CCCOCCCN(C*)C1CC1] | (369) |
| 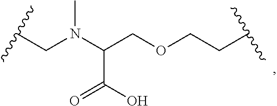 [SMILES: *CCOCC(C(=O)O)N(C)C*] | (370) |
| 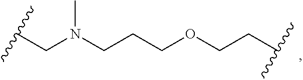 [SMILES: *CCOCCCN(C)C*] | (371) |
| 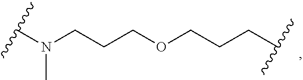 [SMILES: *CCCOCCCN(*)C] | (372) |
| 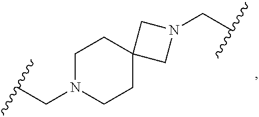 [SMILES: *CN1CCC2(CC1)CN(C*)C2] | (373) |
| 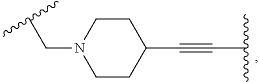 [SMILES: *C#CC1CCN(C*)CC1] | (374) |
| 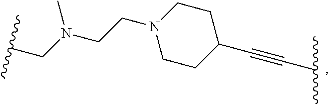 [SMILES: *C#CC1CCN(CCN(C)C*)CC1] | (375) |
| 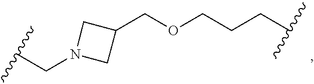 [SMILES: *CCCOCC1CN(C*)C1] | (376) |
| 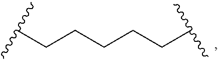 [SMILES: *CCCCC*] | (377) |
| 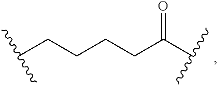 [SMILES: *CCCCC(*)=O] | (378) |
| 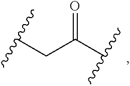 [SMILES: *CC(*)=O] | (379) |
| 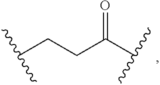 [SMILES: *CCC(*)=O] | (380) |
| 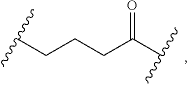 [SMILES: *CCCC(*)=O] | (381) |
| 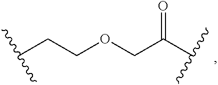 [SMILES: *CCOCC(*)=O] | (382) |
| 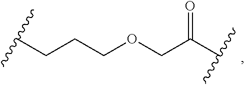 [SMILES: *CCCOCC(*)=O] | (383) |
| 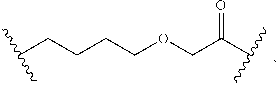 [SMILES: *CCCCOCC(*)=O] | (384) |
| 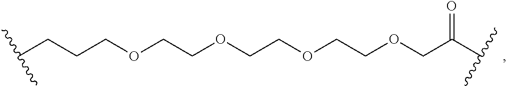 [SMILES: *CCCOCCOCCOCCOCC(*)=O] | (385) |
| 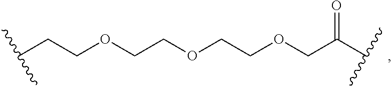 [SMILES: *CCOCCOCCOCC(*)=O] | (386) |
| 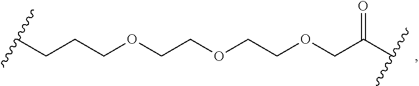 [SMILES: *CCCOCCOCCOCC(*)=O] | (387) |
| 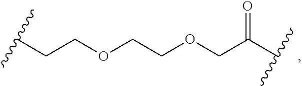 [SMILES: *CCOCCOCC(*)=O] | (388) |
| 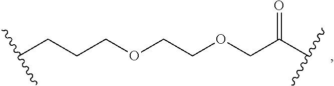 [SMILES: *CCCOCCOCC(*)=O] | (389) |
| 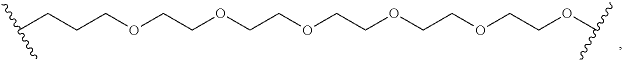 [SMILES: *CCCOCCOCCOCCOCCOCCO*] | (390) |
| 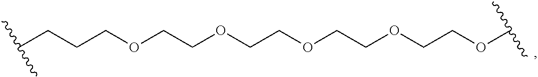 [SMILES: *CCCOCCOCCOCCOCCO*] | (391) |
| 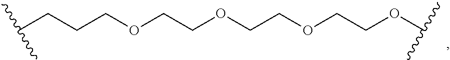 [SMILES: *CCCOCCOCCOCCO*] | (392) |
| 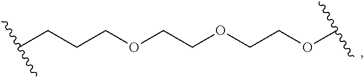 [SMILES: *CCCOCCOCCO*] | (393) |
| 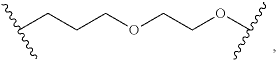 [SMILES: *CCCOCCO*] | (394) |
| 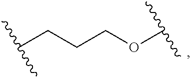 [SMILES: *CCCO*] | (395) |
| 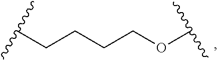 [SMILES: *CCCCO*] | (396) |
| 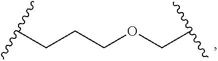 [SMILES: *CCCOC*] | (397) |
| 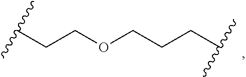 [SMILES: *CCCOCC*] | (398) |
| 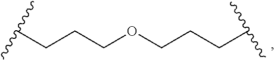 [SMILES: *CCCOCCC*] | (399) |
| 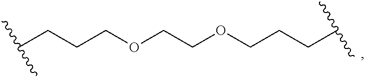 [SMILES: *CCCOCCOCCC*] | (400) |
| 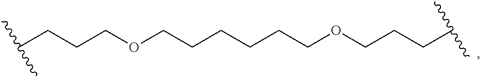 [SMILES: *CCCOCCCCCCOCCC*] | (401) |
| 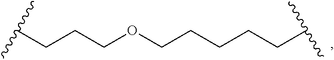 [SMILES: *CCCCCOCCC*] | (402) |
| 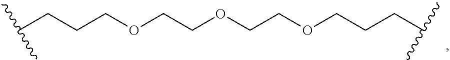 [SMILES: *CCCOCCOCCOCCC*] | (403) |
| 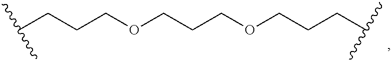 [SMILES: *CCCOCCCOCCC*] | (404) |
| 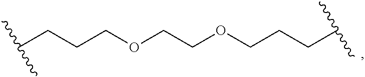 [SMILES: *CCCOCCOCCC*] | (405) |
| 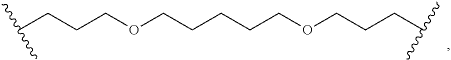 [SMILES: *CCCOCCCCCOCCC*] | (406) |
| 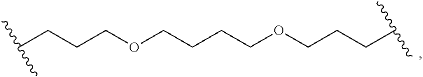 [SMILES: *CCCOCCCCOCCC*] | (407) |
| 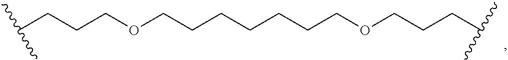 [SMILES: *CCCOCCCCCCCOCCC*] | (408) |
| 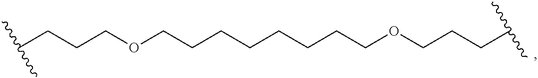 [SMILES: *CCCOCCCCCCCCOCCC*] | (409) |
| 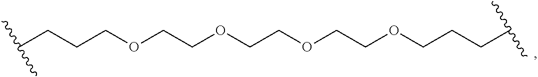 [SMILES: *CCCOCCOCCOCCOCCC*] | (410) |
| 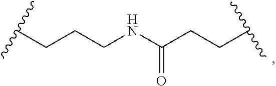 [SMILES: *CCCNC(=O)CC*] | (411) |
| 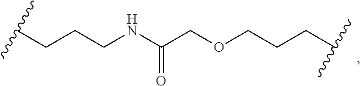 [SMILES: *CCCNC(=O)COCCC*] | (412) |
| 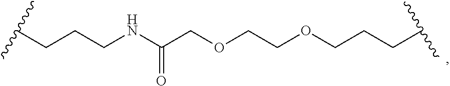 [SMILES: *CCCNC(=O)COCCOCCC*] | (413) |
| 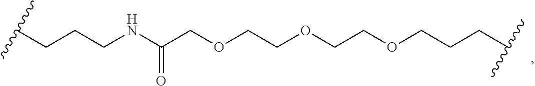 [SMILES: *CCCNC(=O)COCCOCCOCCC*] | (414) |
| 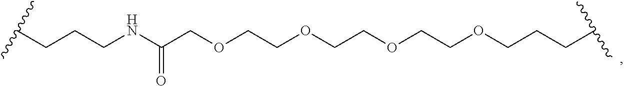 [SMILES: *CCCNC(=O)COCCOCCOCCOCCC*] | (415) |
| 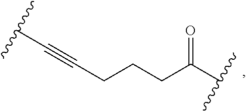 [SMILES: *C#CCCCC(*)=O] | (416) |
| 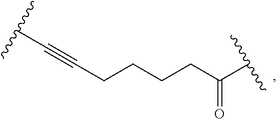 [SMILES: *C#CCCCCC(*)=O] | (417) |
| 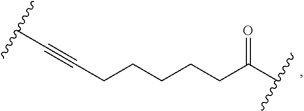 [SMILES: *C#CCCCCCC(*)=O] | (418) |
| 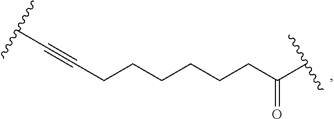 [SMILES: *C#CCCCCCCC(*)=O] | (419) |
| 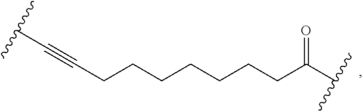 [SMILES: *C#CCCCCCCCC(*)=O] | (420) |
| 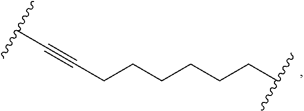 [SMILES: *C#CCCCCCC*] | (421) |
| 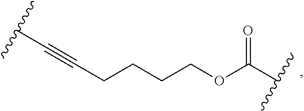 [SMILES: *C#CCCCCOC(*)=O] | (422) |
| 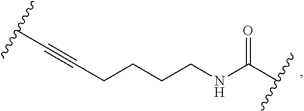 [SMILES: *C#CCCCCNC(*)=O] | (423) |
| 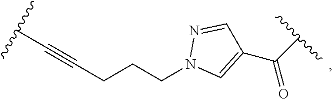 [SMILES: *C#CCCCn1cc(C(*)=O)cn1] | (424) |
| 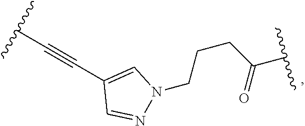 [SMILES: *C#Cc1cnn(CCCC(*)=O)c1] | (425) |
| 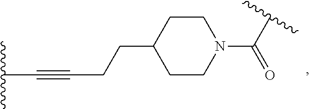 [SMILES: *C#CCCC1CCN(C(*)=O)CC1] | (426) |
| 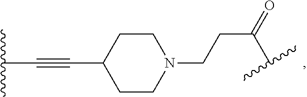 [SMILES: *C#CC1CCN(CCC(*)=O)CC1] | (427) |
| 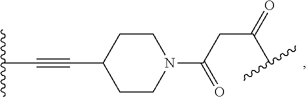 [SMILES: *C#CC1CCN(C(=O)CC(*)=O)CC1] | (428) |
| 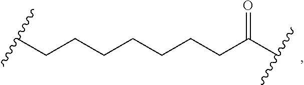 [SMILES: *CCCCCCCC(*)=O] | (429) |
| 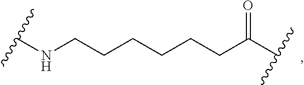 [SMILES: *NCCCCCCC(*)=O] | (430) |
| 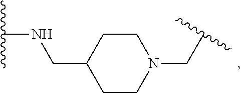 [SMILES: *CN1CCC(CN*)CC1] | (431) |
| 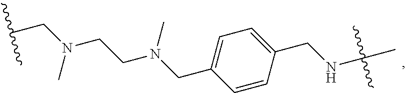 [SMILES: *CN(C)CCN(C)Cc1ccc(CN*)cc1] | (432) |
| 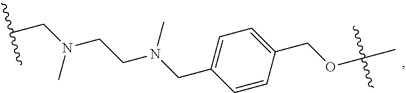 [SMILES: *CN(C)CCN(C)Cc1ccc(CO*)cc1] | (433) |
| 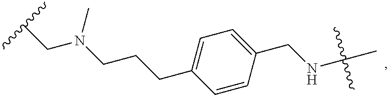 [SMILES: *CN(C)CCCc1ccc(CN*)cc1] | (434) |
| 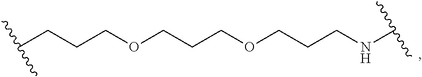 [SMILES: *CCCOCCCOCCCN*] | (435) |
| 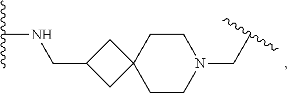 [SMILES: *CN1CCC2(CC1)CC(CN*)C2] | (436) |
| 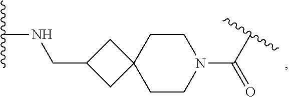 [SMILES: *NCC1CC2(CCN(C(*)=O)CC2)C1] | (437) |
| 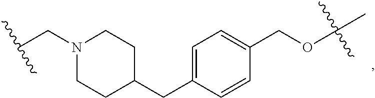 [SMILES: *CN1CCC(Cc2ccc(CO*)cc2)CC1] | (438) |
| 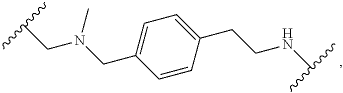 [SMILES: *CN(C)Cc1ccc(CCN*)cc1] | (439) |
| 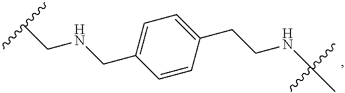 [SMILES: *CNCc1ccc(CCN*)cc1] | (440) |
| 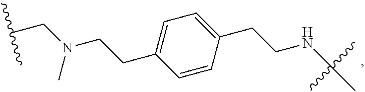 [SMILES: *CN(C)CCc1ccc(CCN*)cc1] | (441) |
| 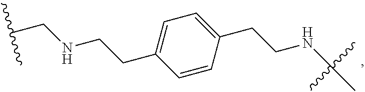 [SMILES: *CNCCc1ccc(CCN*)cc1] | (442) |
| 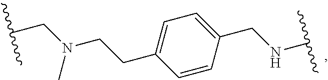 [SMILES: *CN(C)CCc1ccc(CN*)cc1] | (443) |
| 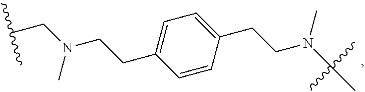 [SMILES: *CN(C)CCc1ccc(CCN(*)C)cc1] | (444) |
| 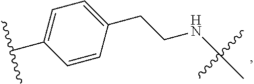 [SMILES: *NCCc1ccc(*)cc1] | (445) |
| 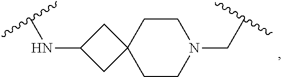 [SMILES: *CN1CCC2(CC1)CC(N*)C2] | (446) |
| 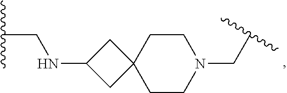 [SMILES: *CNC1CC2(CCN(C*)CC2)C1] | (447) |
| 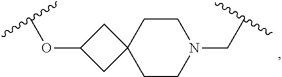 [SMILES: *CN1CCC2(CC1)CC(O*)C2] | (448) |
| 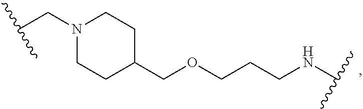 [SMILES: *CN1CCC(COCCCN*)CC1] | (449) |
| 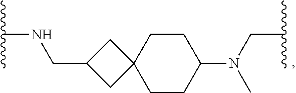 [SMILES: *CN(C)C1CCC2(CC1)CC(CN*)C2] | (450) |
| 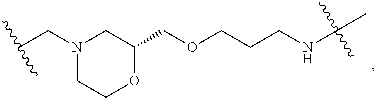 [SMILES: *CN1CCO[C@@H](COCCCN*)C1] | (451) |
| 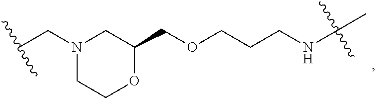 [SMILES: *CN1CCO[C@H](COCCCN*)C1] | (452) |
| 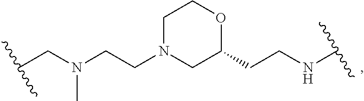 [SMILES: *CN(C)CCN1CCO[C@H](CCN*)C1] | (453) |
| 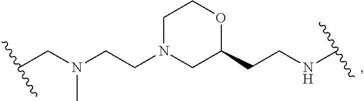 [SMILES: *CN(C)CCN1CCO[C@@H](CCN*)C1] | (454) |
| 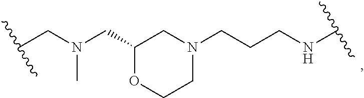 [SMILES: *CN(C)C[C@@H]1CN(CCCN*)CCO1] | (455) |
| 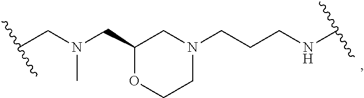 [SMILES: *CN(C)C[C@H]1CN(CCCN*)CCO1] | (456) |
| 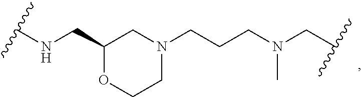 [SMILES: *CN(C)CCCN1CCO[C@@H](CN*)C1] | (457) |
| 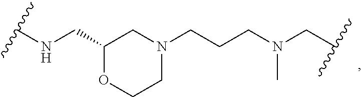 [SMILES: *CN(C)CCCN1CCO[C@H](CN*)C1] | (458) |
| 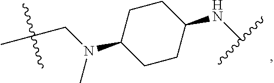 [SMILES: *CN(C)[C@H]1CC[C@@H](N*)CC1] | (459) |
| 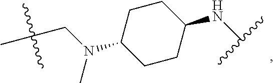 [SMILES: *CN(C)[C@H]1CC[C@H](N*)CC1] | (460) |
| 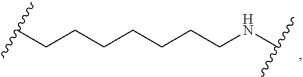 [SMILES: *CCCCCCCN*] | (461) |
| 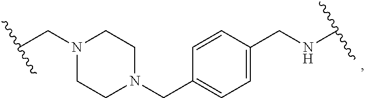 [SMILES: *CN1CCN(Cc2ccc(CN*)cc2)CC1] | (462) |
| 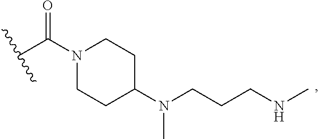 [SMILES: *C(=O)N1CCC(N(C)CCCNC)CC1] | (463) |
| 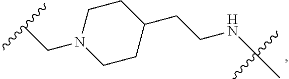 [SMILES: *CN1CCC(CCN*)CC1] | (464) |
| 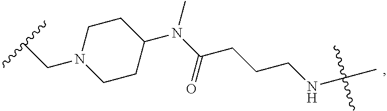 [SMILES: *CN1CCC(N(C)C(=O)CCCN*)CC1] | (465) |
| 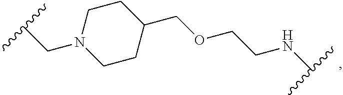 [SMILES: *CN1CCC(COCCN*)CC1] | (466) |
| 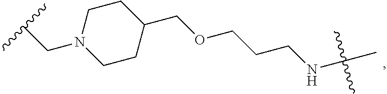 [SMILES: *CN1CCC(COCCCN*)CC1] | (467) |
| 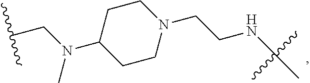 [SMILES: *CN(C)C1CCN(CCN*)CC1] | (468) |
| 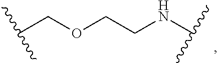 [SMILES: *COCCN*] | (469) |
| 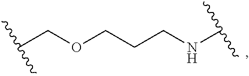 [SMILES: *COCCCN*] | (470) |
| 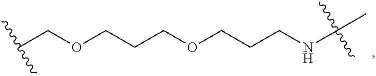 [SMILES: *COCCCOCCCN*] | (471) |
| 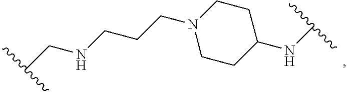 [SMILES: *CNCCCN1CCC(N*)CC1] | (472) |
| 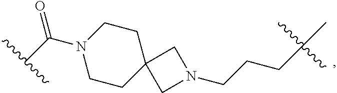 [SMILES: *CCCN1CC2(CCN(C(*)=O)CC2)C1] | (473) |
| 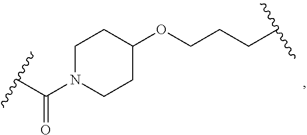 [SMILES: *CCCOC1CCN(C(*)=O)CC1] | (474) |
| 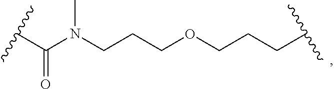 [SMILES: *CCCOCCCN(C)C(*)=O] | (475) |
| 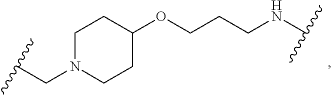 [SMILES: *CN1CCC(OCCCN*)CC1] | (475) |
| 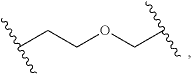 [SMILES: *CCOC*] | (476) |
| 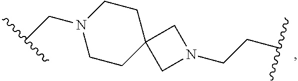 [SMILES: *CCN1CC2(CCN(C*)CC2)C1] | (477) |
| 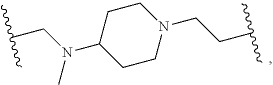 [SMILES: *CCN1CCC(N(C)C*)CC1] | (478) |
| 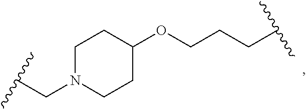 [SMILES: *CCCOC1CCN(C*)CC1] | (479) |
| 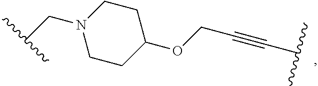 [SMILES: *C#CCOC1CCN(C*)CC1] | (480) |
| 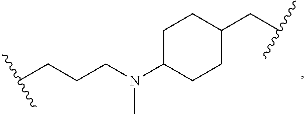 [SMILES: *CCCN(C)C1CCC(C*)CC1] | (481) |
| 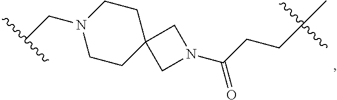 [SMILES: *CCC(=O)N1CC2(CCN(C*)CC2)C1] | (482) |
| 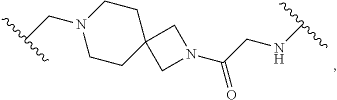 [SMILES: *CN1CCC2(CC1)CN(C(=O)CN*)C2] | (483) |
| 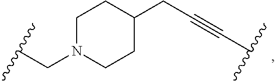 [SMILES: *C#CCC1CCN(C*)CC1] | (484) |
| 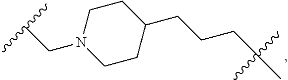 [SMILES: *CCCC1CCN(C*)CC1] | (485) |
| 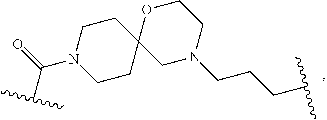 [SMILES: *CCCN1CCOC2(CCN(C(*)=O)CC2)C1] | (486) |
| 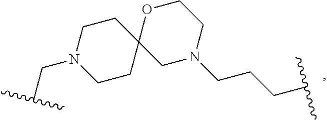 [SMILES: *CCCN1CCOC2(CCN(C*)CC2)C1] | (487) |
| 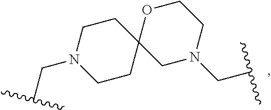 [SMILES: *CN1CCC2(CC1)CN(C*)CCO2] | (488) |
| 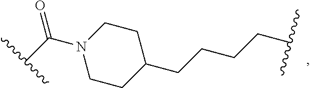 [SMILES: *CCCCC1CCN(C(*)=O)CC1] | (489) |
| 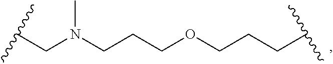 [SMILES: *CCCOCCCN(C)C*] | (490) |
| 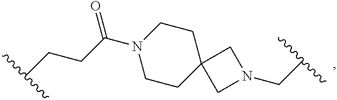 [SMILES: *CCC(=O)N1CCC2(CC1)CN(C*)C2] | (491) |
| 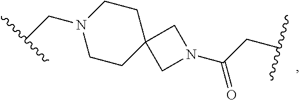 [SMILES: *CC(=O)N1CC2(CCN(C*)CC2)C1] | (492) |
| 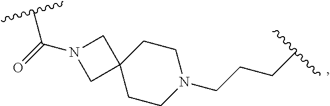 [SMILES: *CCCN1CCC2(CC1)CN(C(*)=O)C2] | (493) |
| 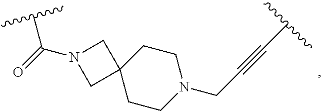 [SMILES: *C#CCN1CCC2(CC1)CN(C(*)=O)C2] | (494) |
| 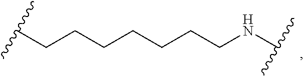 [SMILES: *CCCCCCCN*] | (495) |
| 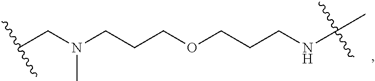 [SMILES: *CN(C)CCCOCCCN*] | (496) |
| 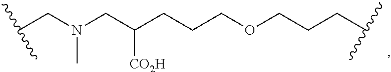 [SMILES: *CCCOCCCC(CN(C)C*)C(=O)O] | (497) |
| 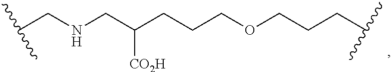 [SMILES: *CCCOCCCC(CNC*)C(=O)O] | (498) |
| 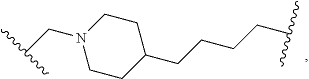 [SMILES: *CCCCC1CCN(C*)CC1] | (499) |
| 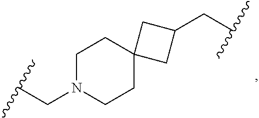 [SMILES: *CC1CC2(CCN(C*)CC2)C1] | (500) |
| 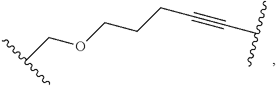 [SMILES: *C#CCCCOC*] | (501) |
| 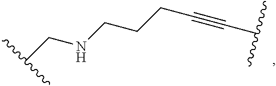 [SMILES: *C#CCCCNC*] | (502) |
| 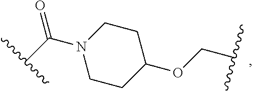 [SMILES: *COC1CCN(C(*)=O)CC1] | (503) |
| 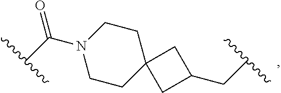 [SMILES: *CC1CC2(CCN(C(*)=O)CC2)C1] | (504) |
| 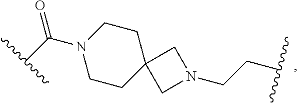 [SMILES: *CCN1CC2(CCN(C(*)=O)CC2)C1] | (505) |
| 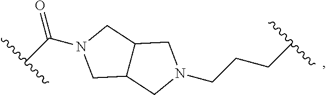 [SMILES: *CCCN1CC2CN(C(*)=O)CC2C1] | (506) |
| 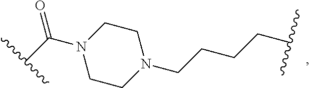 [SMILES: *CCCCN1CCN(C(*)=O)CC1] | (507) |
| 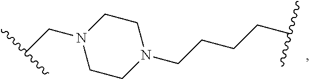 [SMILES: *CCCCN1CCN(C*)CC1] | (508) |
| 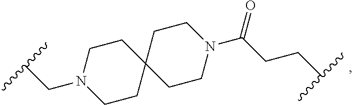 [SMILES: *CCC(=O)N1CCC2(CCN(C*)CC2)CC1] | (509) |
| 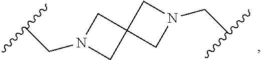 [SMILES: *CN1CC2(C1)CN(C*)C2] | (510) |
| 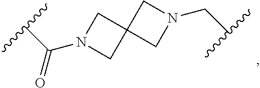 [SMILES: *CN1CC2(C1)CN(C(*)=O)C2] | (511) |
| 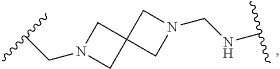 [SMILES: *CN1CC2(C1)CN(CN*)C2] | (512) |
| 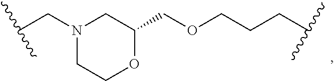 [SMILES: *CCCOC[C@H]1CN(C*)CCO1] | (513) |
| 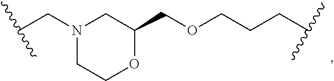 [SMILES: *CCCOC[C@@H]1CN(C*)CCO1] | (514) |
| 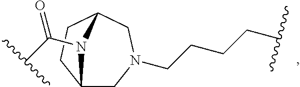 [SMILES: *CCCCN1C[C@H]2CC[C@@H](C1)N2C(*)=O] | (515) |
| 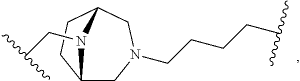 [SMILES: *CCCCN1C[C@H]2CC[C@@H](C1)N2C*] | (516) |
| 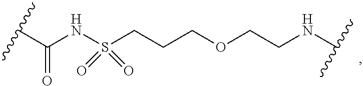 [SMILES: *NCCOCCCS(=O)(=O)NC(*)=O] | (517) |
| 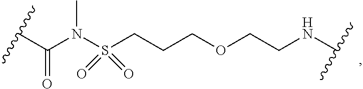 [SMILES: *NCCOCCCS(=O)(=O)N(C)C(*)=O] | (518) |
| 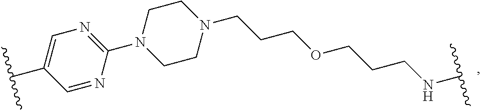 [SMILES: *NCCCOCCCN1CCN(c2ncc(*)cn2)CC1] | (519) |
| 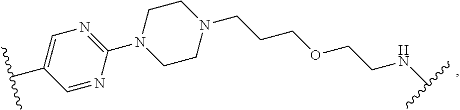 [SMILES: *NCCOCCCN1CCN(c2ncc(*)cn2)CC1] | (520) |
| 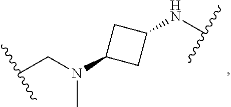 [SMILES: *CN(C)[C@H]1C[C@H](N*)C1] | (521) |
| 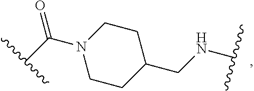 [SMILES: *NCC1CCN(C(*)=O)CC1] | (522) |
| 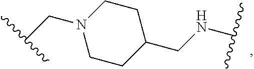 [SMILES: *CN1CCC(CN*)CC1] | (523) |
| 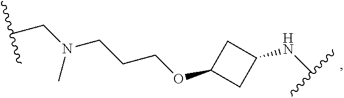 [SMILES: *CN(C)CCCO[C@H]1C[C@H](N*)C1] | (524) |
| 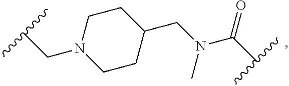 [SMILES: *CN1CCC(CN(C)C(*)=O)CC1] | (525) |
| 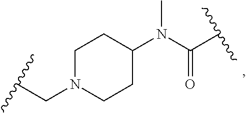 [SMILES: *CN1CCC(N(C)C(*)=O)CC1] | (526) |
| 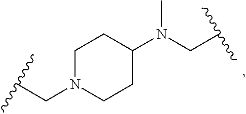 [SMILES: *CN1CCC(N(C)C*)CC1] | (527) |
| 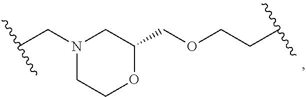 [SMILES: *CCOC[C@H]1CN(C*)CCO1] | (528) |
| 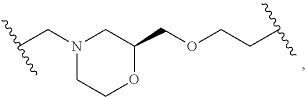 [SMILES: *CCOC[C@@H]1CN(C*)CCO1] | (529) |
| 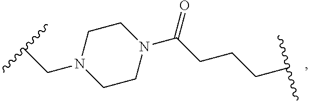 [SMILES: *CCCC(=O)N1CCN(C*)CC1] | (530) |
| 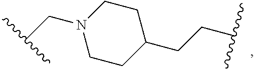 [SMILES: *CCC1CCN(C*)CC1] | (531) |
| 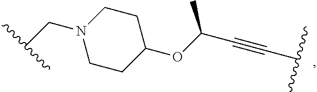 [SMILES: *C#C[C@H](C)OC1CCN(C*)CC1] | (532) |
| 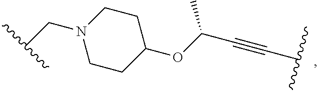 [SMILES: *C#C[C@@H](C)OC1CCN(C*)CC1] | (533) |
| 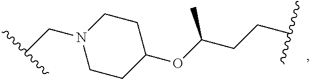 [SMILES: *CC[C@H](C)OC1CCN(C*)CC1] | (534) |
| 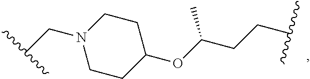 [SMILES: *CC[C@@H](C)OC1CCN(C*)CC1] | (535) |
| 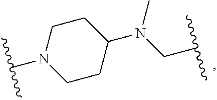 [SMILES: *CN(C)C1CCN(*)CC1] | (536) |
| 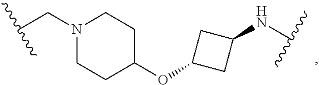 [SMILES: *CN1CCC(O[C@H]2C[C@H](N*)C2)CC1] | (537) |
| 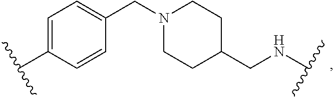 [SMILES: *NCC1CCN(Cc2ccc(*)cc2)CC1] | (538) |
| 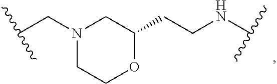 [SMILES: *CN1CCO[C@@H](CCN*)C1] | (539) |
| 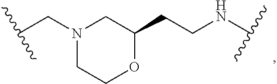 [SMILES: *CN1CCO[C@H](CCN*)C1] | (540) |
| 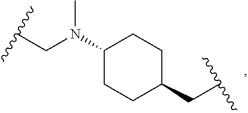 [SMILES: *CN(C)[C@H]1CC[C@H](C*)CC1] | (541) |
| 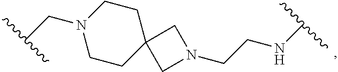 [SMILES: *CN1CCC2(CC1)CN(CCN*)C2] | (542) |
| 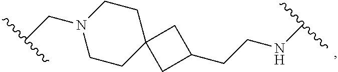 [SMILES: *CN1CCC2(CC1)CC(CCN*)C2] | (543) |
| 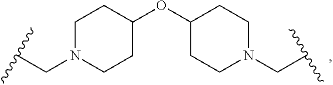 [SMILES: *CN1CCC(OC2CCN(C*)CC2)CC1] | (544) |
| 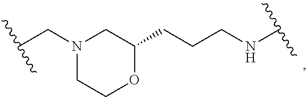 [SMILES: *CN1CCO[C@@H](CCCN*)C1] | (545) |
| 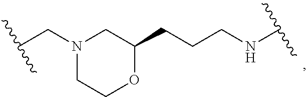 [SMILES: *CN1CCO[C@H](CCCN*)C1] | (546) |
| 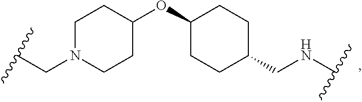 [SMILES: *CN1CCC(O[C@H]2CC[C@H](CN*)CC2)CC1] | (547) |
| 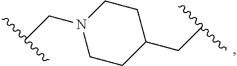 [SMILES: *CC1CCN(C*)CC1] | (548) |
| 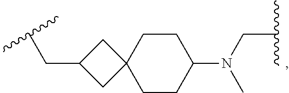 [SMILES: *CC1CC2(CCC(N(C)C*)CC2)C1] | (549) |
| 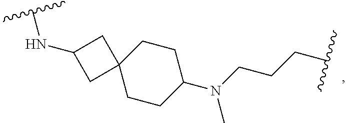 [SMILES: *CCCN(C)C1CCC2(CC1)CC(N*)C2] | (550) |
| 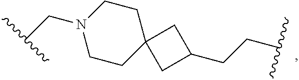 [SMILES: *CCC1CC2(CCN(C*)CC2)C1] | (551) |
| 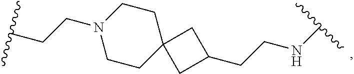 [SMILES: *CCN1CCC2(CC1)CC(CCN*)C2] | (552) |
| 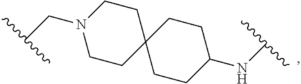 [SMILES: *CN1CCC2(CCC(N*)CC2)CC1] | (553) |
| 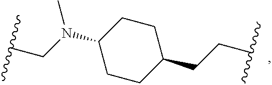 [SMILES: *CC[C@H]1CC[C@H](N(C)C*)CC1] | (554) |
| 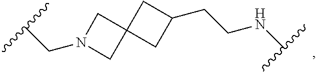 [SMILES: *CN1CC2(CC(CCN*)C2)C1] | (555) |
| 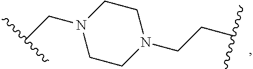 [SMILES: *CCN1CCN(C*)CC1] | (556) |
| 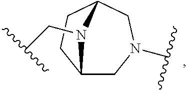 [SMILES: *CN1[C@@H]2CC[C@H]1CN(*)C2] | (557) |
| 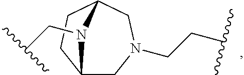 [SMILES: *CCN1C[C@H]2CC[C@@H](C1)N2C*] | (558) |
| 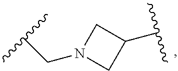 [SMILES: *CN1CC(*)C1] | (559) |
| 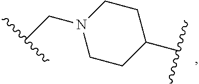 [SMILES: *CN1CCC(*)CC1] | (560) |
| 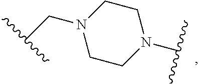 [SMILES: *CN1CCN(*)CC1] | (561) |
| 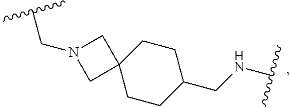 [SMILES: *CN1CC2(CCC(CN*)CC2)C1] | (562) |
| 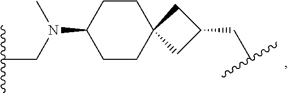 | (563) |
| 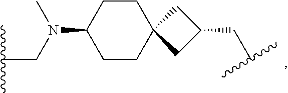 | (564) |
| 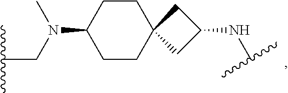 | (565) |
| 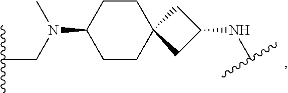 | (566) |
| 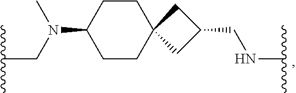 | (567) |
| 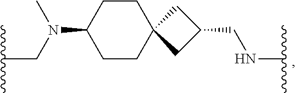 | (568) |
| 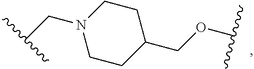 [SMILES: *CN1CCC(CO*)CC1] | (569) |
| 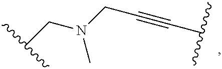 [SMILES: *C#CCN(C)C*] | (570) |
| 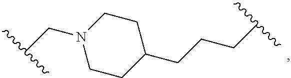 [SMILES: *CCCC1CCN(C*)CC1] | (571) |
| 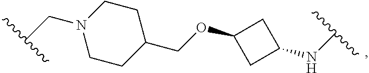 [SMILES: *CN1CCC(CO[C@H]2C[C@H](N*)C2)CC1] | (572) |
| 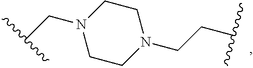 [SMILES: *CCN1CCN(C*)CC1] | (573) |
| 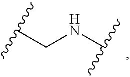 [SMILES: *CN*] | (574) |
| 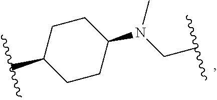 [SMILES: *CN(C)[C@H]1CC[C@@H](*)CC1] | (575) |
| 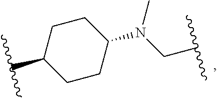 [SMILES: *CN(C)[C@H]1CC[C@H](*)CC1] | (576) |
| 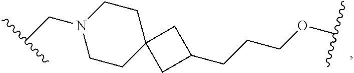 [SMILES: *CN1CCC2(CC1)CC(CCCO*)C2] | (577) |
| 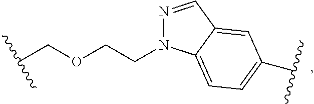 [SMILES: *COCCn1ncc2cc(*)ccc21] | (578) |
| 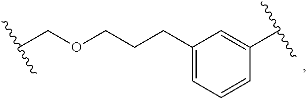 [SMILES: *COCCCc1cccc(*)c1] | (579) |
| 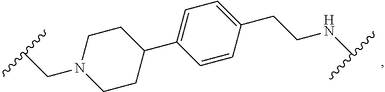 [SMILES: *CN1CCC(c2ccc(CCN*)cc2)CC1] | (580) |
| 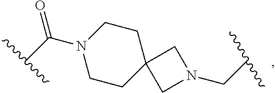 [SMILES: *CN1CC2(CCN(C(*)=O)CC2)C1] | (581) |
| 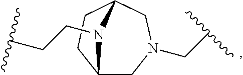 [SMILES: *CCN1[C@@H]2CC[C@H]1CN(C*)C2] | (582) |
| 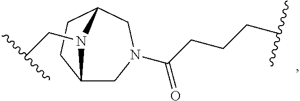 [SMILES: *CCCC(=O)N1C[C@H]2CC[C@@H](C1)N2C*] | (583) |
| 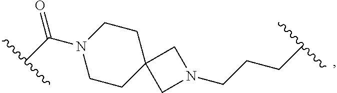 [SMILES: *CCCN1CC2(CCN(C(*)=O)CC2)C1] | (584) |
| 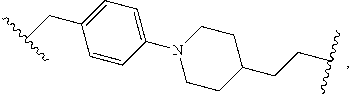 [SMILES: *CCC1CCN(c2ccc(C*)cc2)CC1] | (585) |
| 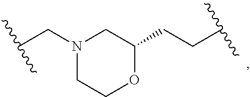 [SMILES: *CC[C@H]1CN(C*)CCO1] | (586) |
| 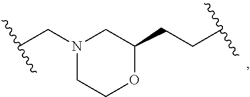 [SMILES: *CC[C@@H]1CN(C*)CCO1] | (587) |
| 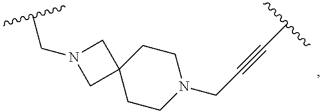 [SMILES: *C#CCN1CCC2(CC1)CN(C*)C2] | (588) |
| 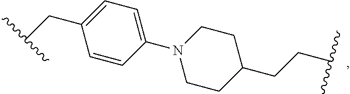 [SMILES: *CCC1CCN(c2ccc(C*)cc2)CC1] | (589) |
| 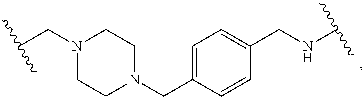 [SMILES: *CN1CCN(Cc2ccc(CN*)cc2)CC1] | (590) |
| 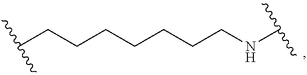 [SMILES: *CCCCCCCN*] | (591) |
| 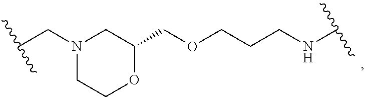 [SMILES: *CN1CCO[C@@H](COCCCN*)C1] | (592) |
| 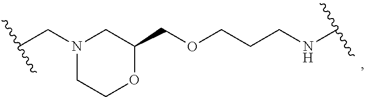 [SMILES: *CN1CCO[C@H](COCCCN*)C1] | (593) |
| 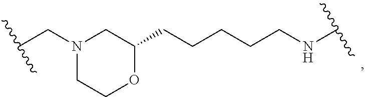 [SMILES: *CN1CCO[C@@H](CCCCCN*)C1] | (594) |
| 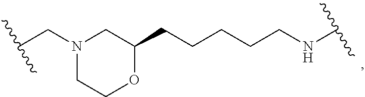 [SMILES: *CN1CCO[C@H](CCCCCN*)C1] | (595) |
| 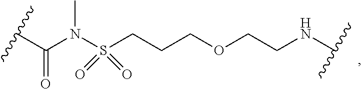 [SMILES: *NCCOCCCS(=O)(=O)N(C)C(*)=O] | (596) |
| 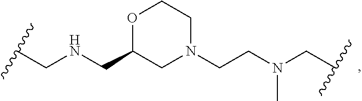 [SMILES: *CNC[C@@H]1CN(CCN(C)C*)CCO1] | (597) |
| 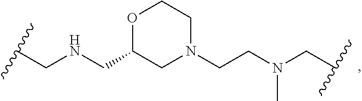 [SMILES: *CNC[C@H]1CN(CCN(C)C*)CCO1] | (598) |
| 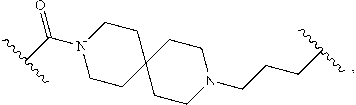 [SMILES: *CCCN1CCC2(CC1)CCN(C(*)=O)CC2] | (599) |
| 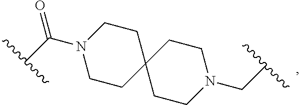 [SMILES: *CN1CCC2(CC1)CCN(C(*)=O)CC2] | (600) |
| 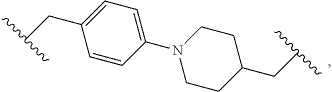 [SMILES: *Cc1ccc(N2CCC(C*)CC2)cc1] | (601) |
| 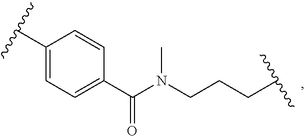 [SMILES: *CCCN(C)C(=O)c1ccc(*)cc1] | (602) |
| 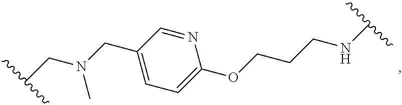 [SMILES: *CN(C)Cc1ccc(OCCCN*)nc1] | (603) |
| 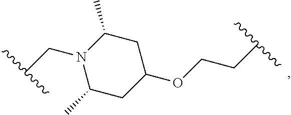 [SMILES: *CCOC1C[C@@H](C)N(C*)[C@@H](C)C1] | (604) |
| 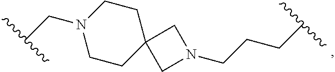 [SMILES: *CCCN1CC2(CCN(C*)CC2)C1] | (605) |
| 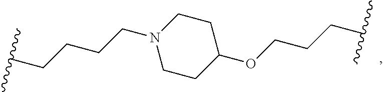 [SMILES: *CCCCN1CCC(OCCC*)CC1] | (606) |
| and | |
| 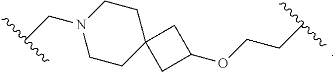 [SMILES: *CCOC1CC2(CCN(C*)CC2)C1] | (607) |

In some embodiments, the present invention provides a compound having an UBM binding moiety described and disclosed herein, an TBM set forth in Table A above, and a linker set forth in Table B above, or a pharmaceutically acceptable salt thereof.

Exemplary compounds of the invention are set forth in Table 1A and 1B, below.

TABLE 1A
Exemplary Compounds

| I-# | Structure |
|---|---|
| I-1 | 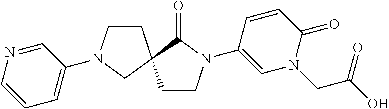 [SMILES: O=C(O)Cn1cc(N2CC[C@]3(CCN(c4cccnc4)C3)C2=O)ccc1=O] |
| I-2 | 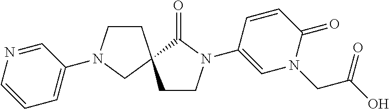 [SMILES: O=C(O)Cn1cc(N2CC[C@@]3(CCN(c4cccnc4)C3)C2=O)ccc1=O] |
| I-3 | 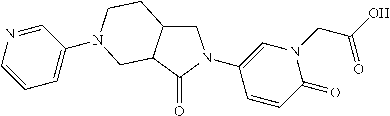 [SMILES: O=C(O)Cn1cc(N2CC3CCN(c4cccnc4)CC3C2=O)ccc1=O] |
| I-4 | 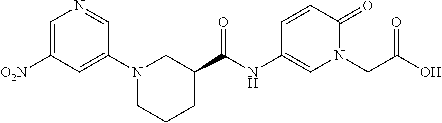 [SMILES: O=C(O)Cn1cc(NC(=O)[C@H]2CCCN(c3cncc([N+](=O)[O-])c3)C2)ccc1=O] |
| I-5 | 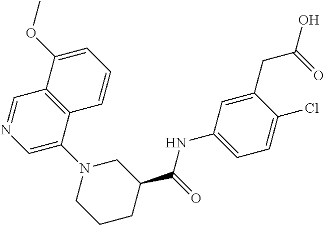 [SMILES: COc1cccc2c(N3CCC[C@H](C(=O)Nc4ccc(Cl)c(CC(=O)O)c4)C3)cncc12] |
| I-6 | 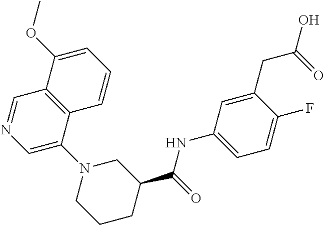 [SMILES: COc1cccc2c(N3CCC[C@H](C(=O)Nc4ccc(F)c(CC(=O)O)c4)C3)cncc12] |
| I-7 | 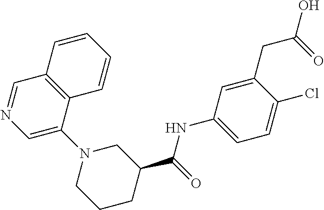 [SMILES: O=C(O)Cc1cc(NC(=O)[C@H]2CCCN(c3cncc4ccccc34)C2)ccc1Cl] |
| I-8 | 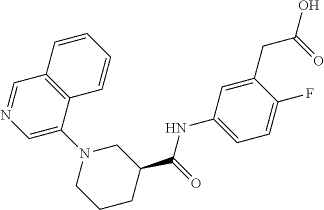 [SMILES: O=C(O)Cc1cc(NC(=O)[C@H]2CCCN(c3cncc4ccccc34)C2)ccc1F] |
| I-9 | 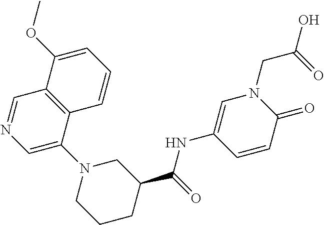 [SMILES: COc1cccc2c(N3CCC[C@H](C(=O)Nc4ccc(=O)n(CC(=O)O)c4)C3)cncc12] |
| I-10 | 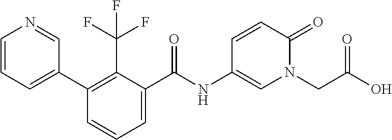 [SMILES: O=C(O)Cn1cc(NC(=O)c2cccc(-c3cccnc3)c2C(F)(F)F)ccc1=O] |
| I-11 | 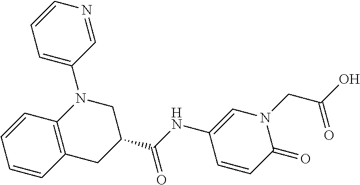 [SMILES: O=C(O)Cn1cc(NC(=O)[C@@H]2Cc3ccccc3N(c3cccnc3)C2)ccc1=O] |
| I-12 | 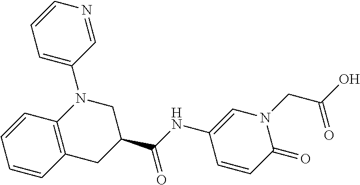 [SMILES: O=C(O)Cn1cc(NC(=O)[C@H]2Cc3ccccc3N(c3cccnc3)C2)ccc1=O] |
| I-13 | 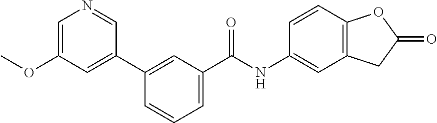 [SMILES: COc1cncc(-c2cccc(C(=O)Nc3ccc4c(c3)CC(=O)O4)c2)c1] |
| I-14 | 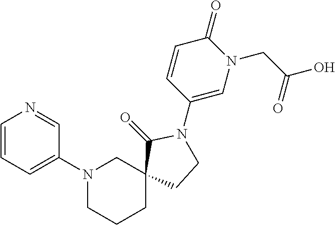 [SMILES: O=C(O)Cn1cc(N2CC[C@@]3(CCCN(c4cccnc4)C3)C2=O)ccc1=O] |
| I-15 | 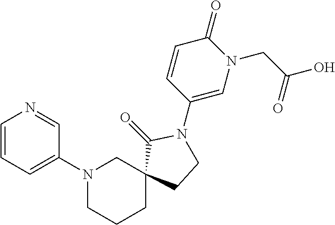 [SMILES: O=C(O)Cn1cc(N2CC[C@]3(CCCN(c4cccnc4)C3)C2=O)ccc1=O] |
| I-16 | 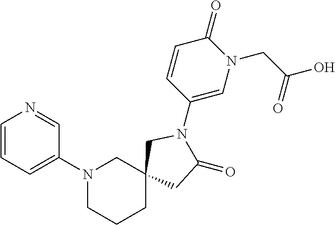 [SMILES: O=C(O)Cn1cc(N2C[C@]3(CCCN(c4cccnc4)C3)CC2=O)ccc1=O] |
| I-17 | 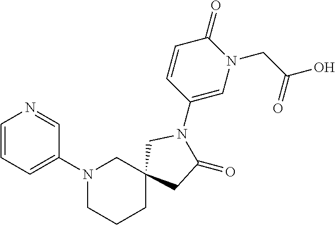 [SMILES: O=C(O)Cn1cc(N2C[C@@]3(CCCN(c4cccnc4)C3)CC2=O)ccc1=O] |
| I-18 | 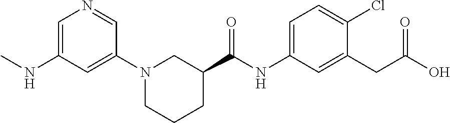 [SMILES: CNc1cncc(N2CCC[C@H](C(=O)Nc3ccc(Cl)c(CC(=O)O)c3)C2)c1] |
| I-19 | 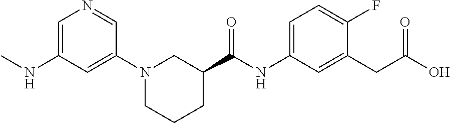 [SMILES: CNc1cncc(N2CCC[C@H](C(=O)Nc3ccc(F)c(CC(=O)O)c3)C2)c1] |
| I-20 | 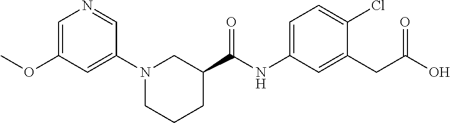 [SMILES: COc1cncc(N2CCC[C@H](C(=O)Nc3ccc(Cl)c(CC(=O)O)c3)C2)c1] |
| I-21 | 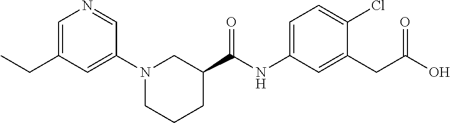 [SMILES: CCc1cncc(N2CCC[C@H](C(=O)Nc3ccc(Cl)c(CC(=O)O)c3)C2)c1] |
| I-22 | 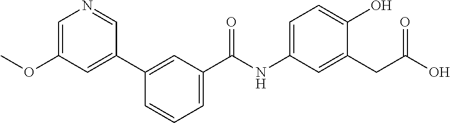 [SMILES: COc1cncc(-c2cccc(C(=O)Nc3ccc(O)c(CC(=O)O)c3)c2)c1] |
| I-23 | 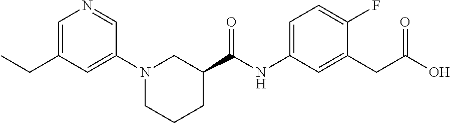 [SMILES: CCc1cncc(N2CCC[C@H](C(=O)Nc3ccc(F)c(CC(=O)O)c3)C2)c1] |
| I-24 | 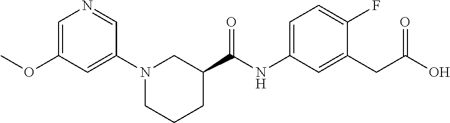 [SMILES: COc1cncc(N2CCC[C@H](C(=O)Nc3ccc(F)c(CC(=O)O)c3)C2)c1] |
| I-25 | 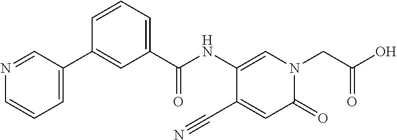 [SMILES: N#Cc1cc(=O)n(CC(=O)O)cc1NC(=O)c1cccc(-c2cccnc2)c1] |
| I-26 | 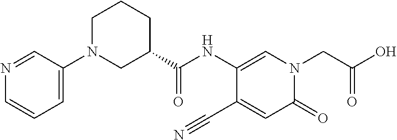 [SMILES: N#Cc1cc(=O)n(CC(=O)O)cc1NC(=O)[C@H]1CCCN(c2cccnc2)C1] |
| I-27 | 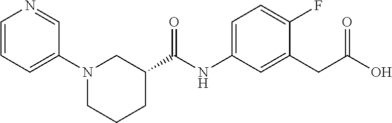 [SMILES: O=C(O)Cc1cc(NC(=O)[C@@H]2CCCN(c3cccnc3)C2)ccc1F] |
| I-28 | 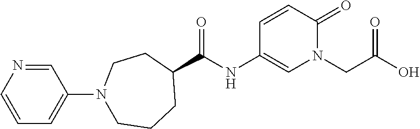 [SMILES: O=C(O)Cn1cc(NC(=O)[C@H]2CCCN(c3cccnc3)CC2)ccc1=O] |
| I-29 | 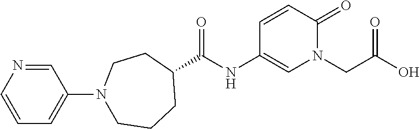 [SMILES: O=C(O)Cn1cc(NC(=O)[C@@H]2CCCN(c3cccnc3)CC2)ccc1=O] |
| I-30 | 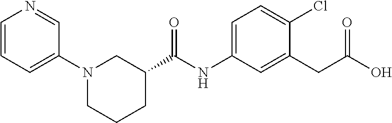 [SMILES: O=C(O)Cc1cc(NC(=O)[C@@H]2CCCN(c3cccnc3)C2)ccc1Cl] |
| I-31 | 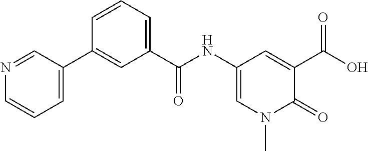 [SMILES: Cn1cc(NC(=O)c2cccc(-c3cccnc3)c2)cc(C(=O)O)c1=O] |
| I-32 | 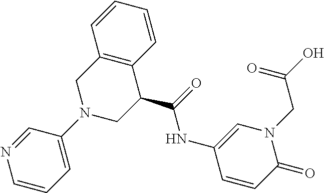 [SMILES: O=C(O)Cn1cc(NC(=O)[C@H]2CN(c3cccnc3)Cc3ccccc32)ccc1=O] |
| I-33 | 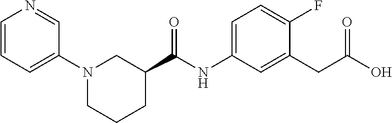 [SMILES: O=C(O)Cc1cc(NC(=O)[C@H]2CCCN(c3cccnc3)C2)ccc1F] |
| I-34 | 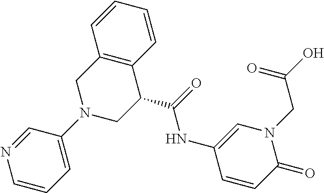 [SMILES: O=C(O)Cn1cc(NC(=O)[C@@H]2CN(c3cccnc3)Cc3ccccc32)ccc1=O] |
| I-35 | 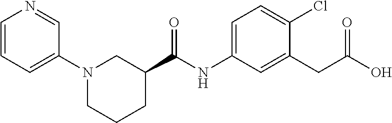 [SMILES: O=C(O)Cc1cc(NC(=O)[C@H]2CCCN(c3cccnc3)C2)ccc1Cl] |
| I-36 | 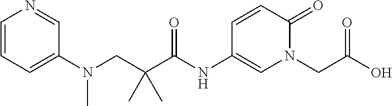 [SMILES: CN(CC(C)(C)C(=O)Nc1ccc(=O)n(CC(=O)O)c1)c1cccnc1] |
| I-37 | 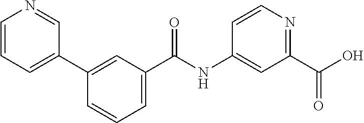 [SMILES: O=C(Nc1ccnc(C(=O)O)c1)c1cccc(-c2cccnc2)c1] |
| I-38 | 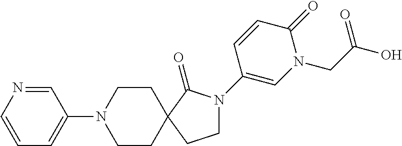 [SMILES: O=C(O)Cn1cc(N2CCC3(CCN(c4cccnc4)CC3)C2=O)ccc1=O] |
| I-39 | 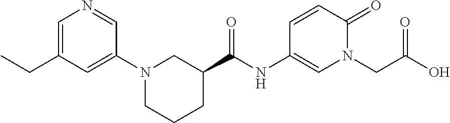 [SMILES: CCc1cncc(N2CCC[C@H](C(=O)Nc3ccc(=O)n(CC(=O)O)c3)C2)c1] |
| I-40 | 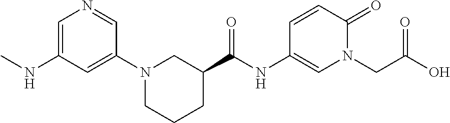 [SMILES: CNc1cncc(N2CCC[C@H](C(=O)Nc3ccc(=O)n(CC(=O)O)c3)C2)c1] |
| I-41 | 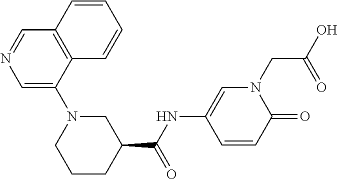 [SMILES: O=C(O)Cn1cc(NC(=O)[C@H]2CCCN(c3cncc4ccccc34)C2)ccc1=O] |
| I-42 | 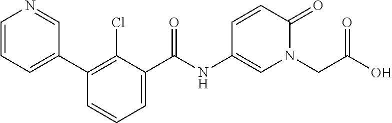 [SMILES: O=C(O)Cn1cc(NC(=O)c2cccc(-c3cccnc3)c2Cl)ccc1=O] |
| I-413 | 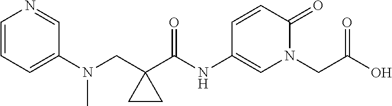 [SMILES: CN(CC1(C(=O)Nc2ccc(=O)n(CC(=O)O)c2)CC1)c1cccnc1] |
| I-44 | 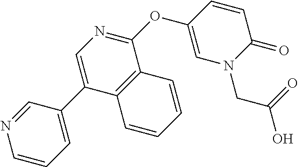 [SMILES: O=C(O)Cn1cc(Oc2ncc(-c3cccnc3)c3ccccc23)ccc1=O] |
| I-45 | 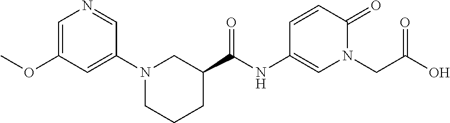 [SMILES: COc1cncc(N2CCC[C@H](C(=O)Nc3ccc(=O)n(CC(=O)O)c3)C2)c1] |
| I-46 | 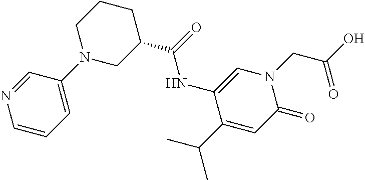 [SMILES: CC(C)c1cc(=O)n(CC(=O)O)cc1NC(=O)[C@H]1CCCN(c2cccnc2)C1] |
| I-47 | 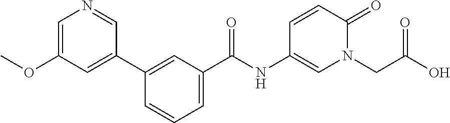 [SMILES: COc1cncc(-c2cccc(C(=O)Nc3ccc(=O)n(CC(=O)O)c3)c2)c1] |
| I-48 | 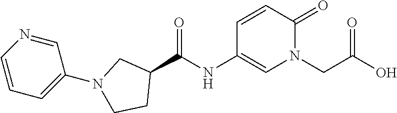 [SMILES: O=C(O)Cn1cc(NC(=O)[C@H]2CCN(c3cccnc3)C2)ccc1=O] |
| I-49 | 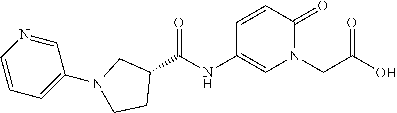 [SMILES: O=C(O)Cn1cc(NC(=O)[C@@H]2CCN(c3cccnc3)C2)ccc1=O] |
| I-50 | 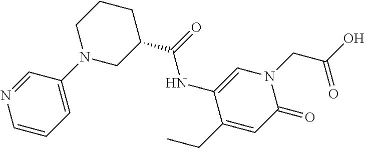 [SMILES: CCc1cc(=O)n(CC(=O)O)cc1NC(=O)[C@H]1CCCN(c2cccnc2)C1] |
| I-51 | 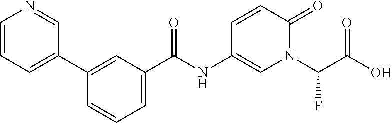 [SMILES: O=C(Nc1ccc(=O)n([C@@H](F)C(=O)O)c1)c1cccc(-c2cccnc2)c1] |
| I-52 | 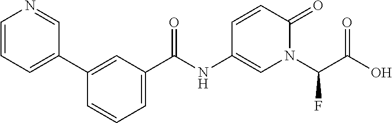 [SMILES: O=C(Nc1ccc(=O)n([C@H](F)C(=O)O)c1)c1cccc(-c2cccnc2)c1] |
| I-53 | 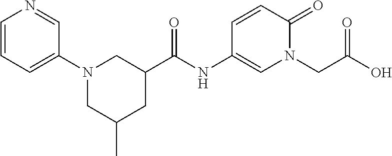 [SMILES: CC1CC(C(=O)Nc2ccc(=O)n(CC(=O)O)c2)CN(c2cccnc2)C1] |
| I-54 | 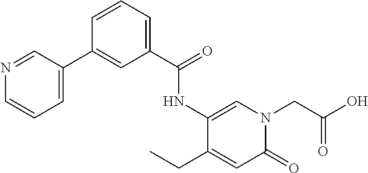 [SMILES: CCc1cc(=O)n(CC(=O)O)cc1NC(=O)c1cccc(-c2cccnc2)c1] |
| I-55 | 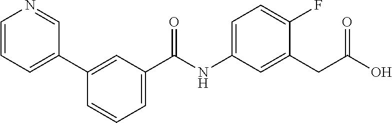 [SMILES: O=C(O)Cc1cc(NC(=O)c2cccc(-c3cccnc3)c2)ccc1F] |
| I-56 | 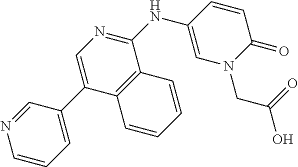 [SMILES: O=C(O)Cn1cc(Nc2ncc(-c3cccnc3)c3ccccc23)ccc1=O] |
| I-57 | 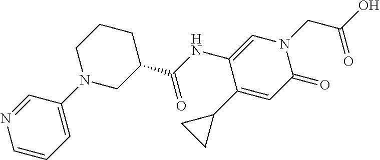 [SMILES: O=C(O)Cn1cc(NC(=O)[C@H]2CCCN(c3cccnc3)C2)c(C2CC2)cc1=O] |
| I-58 | 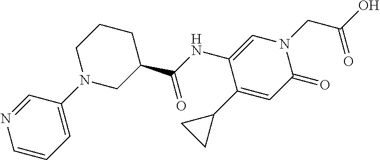 [SMILES: O=C(O)Cn1cc(NC(=O)[C@@H]2CCCN(c3cccnc3)C2)c(C2CC2)cc1=O] |
| I-59 | 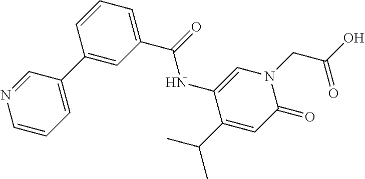 [SMILES: CC(C)c1cc(=O)n(CC(=O)O)cc1NC(=O)c1cccc(-c2cccnc2)c1] |
| I-60 | 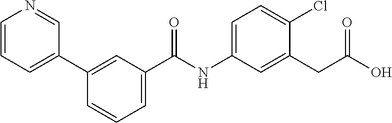 [SMILES: O=C(O)Cc1cc(NC(=O)c2cccc(-c3cccnc3)c2)ccc1Cl] |
| I-61 | 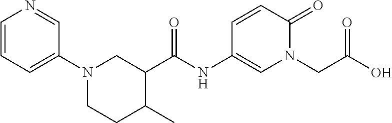 [SMILES: CC1CCN(c2cccnc2)CC1C(=O)Nc1ccc(=O)n(CC(=O)O)c1] |
| I-62 | 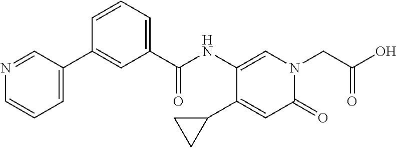 [SMILES: O=C(O)Cn1cc(NC(=O)c2cccc(-c3cccnc3)c2)c(C2CC2)cc1=O] |
| I-63 | 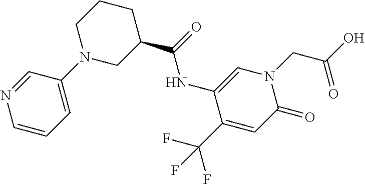 [SMILES: O=C(O)Cn1cc(NC(=O)[C@@H]2CCCN(c3cccnc3)C2)c(C(F)(F)F)cc1=O] |
| I-64 | 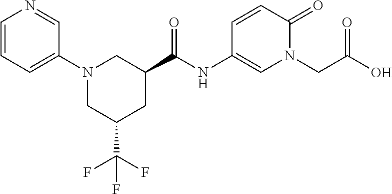 [SMILES: O=C(O)Cn1cc(NC(=O)[C@H]2C[C@H](C(F)(F)F)CN(c3cccnc3)C2)ccc1=O] |
| I-65 | 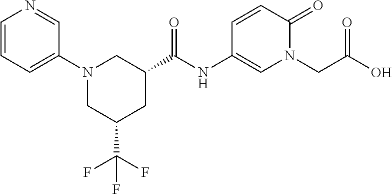 [SMILES: O=C(O)Cn1cc(NC(=O)[C@@H]2C[C@H](C(F)(F)F)CN(c3cccnc3)C2)ccc1=O] |
| I-66 | 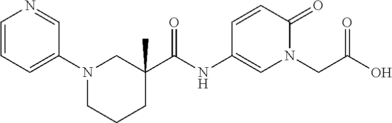 [SMILES: C[C@@]1(C(=O)Nc2ccc(=O)n(CC(=O)O)c2)CCCN(c2cccnc2)C1] |
| I-67 | 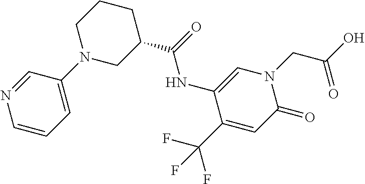 [SMILES: O=C(O)Cn1cc(NC(=O)[C@H]2CCCN(c3cccnc3)C2)c(C(F)(F)F)cc1=O] |
| I-68 | 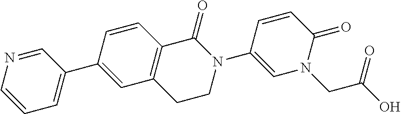 [SMILES: O=C(O)Cn1cc(N2CCc3cc(-c4cccnc4)ccc3C2=O)ccc1=O] |
| I-69 | 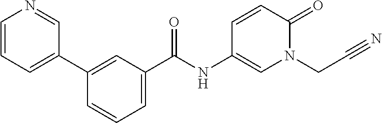 [SMILES: N#CCn1cc(NC(=O)c2cccc(-c3cccnc3)c2)ccc1=O] |
| I-70 | 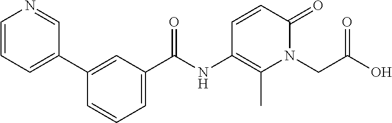 [SMILES: Cc1c(NC(=O)c2cccc(-c3cccnc3)c2)ccc(=O)n1CC(=O)O] |
| I-71 | 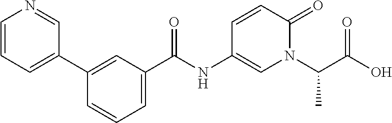 [SMILES: C[C@@H](C(=O)O)n1cc(NC(=O)c2cccc(-c3cccnc3)c2)ccc1=O] |
| I-72 | 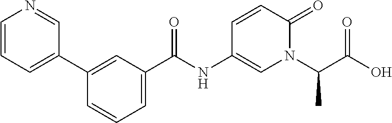 [SMILES: C[C@H](C(=O)O)n1cc(NC(=O)c2cccc(-c3cccnc3)c2)ccc1=O] |
| I-73 | 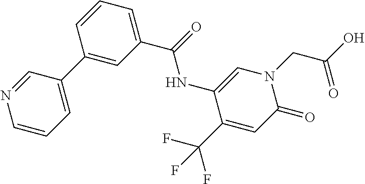 [SMILES: O=C(O)Cn1cc(NC(=O)c2cccc(-c3cccnc3)c2)c(C(F)(F)F)cc1=O] |
| I-74 | 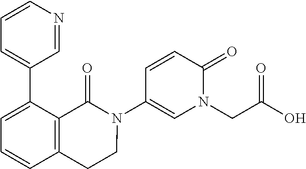 [SMILES: O=C(O)Cn1cc(N2CCc3cccc(-c4cccnc4)c3C2=O)ccc1=O] |
| I-75 | 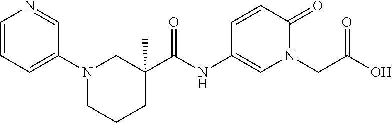 [SMILES: C[C@]1(C(=O)Nc2ccc(=O)n(CC(=O)O)c2)CCCN(c2cccnc2)C1] |
| I-76 | 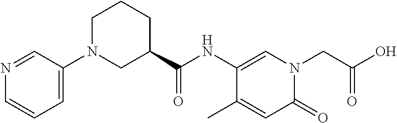 [SMILES: Cc1cc(=O)n(CC(=O)O)cc1NC(=O)[C@@H]1CCCN(c2cccnc2)C1] |
| I-77 | 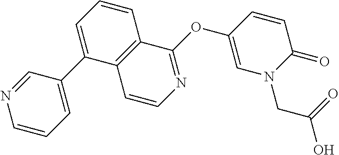 [SMILES: O=C(O)Cn1cc(Oc2nccc3c(-c4cccnc4)cccc23)ccc1=O] |
| I-78 | 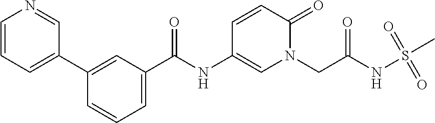 [SMILES: CS(=O)(=O)NC(=O)Cn1cc(NC(=O)c2cccc(-c3cccnc3)c2)ccc1=O] |
| I-79 | 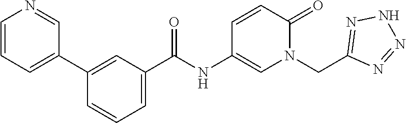 [SMILES: O=C(Nc1ccc(=O)n(Cc2nn[nH]n2)c1)c1cccc(-c2cccnc2)c1] |
| I-80 | 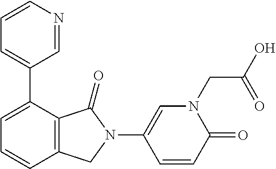 [SMILES: O=C(O)Cn1cc(N2Cc3cccc(-c4cccnc4)c3C2=O)ccc1=O] |
| I-81 | 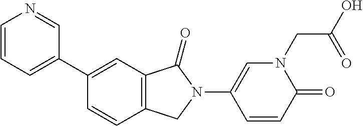 [SMILES: O=C(O)Cn1cc(N2Cc3ccc(-c4cccnc4)cc3C2=O)ccc1=O] |
| I-82 | 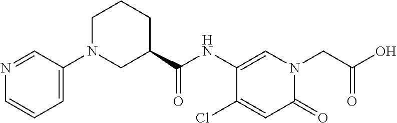 [SMILES: O=C(O)Cn1cc(NC(=O)[C@@H]2CCCN(c3cccnc3)C2)c(Cl)cc1=O] |
| I-83 | 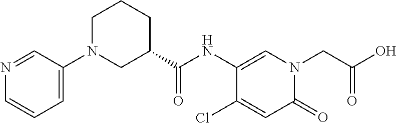 [SMILES: O=C(O)Cn1cc(NC(=O)[C@H]2CCCN(c3cccnc3)C2)c(Cl)cc1=O] |
| I-84 | 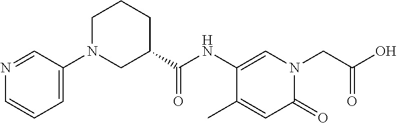 [SMILES: Cc1cc(=O)n(CC(=O)O)cc1NC(=O)[C@H]1CCCN(c2cccnc2)C1] |
| I-85 | 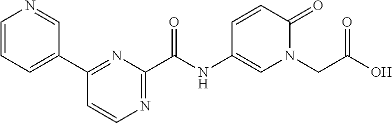 [SMILES: O=C(O)Cn1cc(NC(=O)c2nccc(-c3cccnc3)n2)ccc1=O] |
| I-86 | 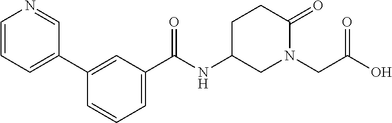 [SMILES: O=C(O)CN1CC(NC(=O)c2cccc(-c3cccnc3)c2)CCC1=O] |
| I-87 | 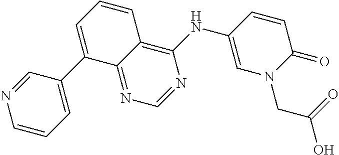 [SMILES: O=C(O)Cn1cc(Nc2ncnc3c(-c4cccnc4)cccc23)ccc1=O] |
| I-88 | 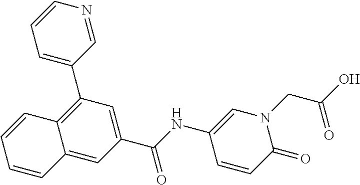 [SMILES: O=C(O)Cn1cc(NC(=O)c2cc(-c3cccnc3)c3ccccc3c2)ccc1=O] |
| I-89 | 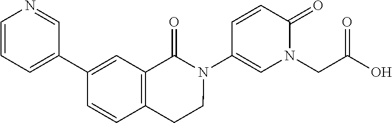 [SMILES: O=C(O)Cn1cc(N2CCc3ccc(-c4cccnc4)cc3C2=O)ccc1=O] |
| I-90 | 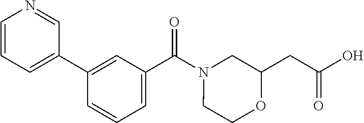 [SMILES: O=C(O)CC1CN(C(=O)c2cccc(-c3cccnc3)c2)CCO1] |
| I-91 | 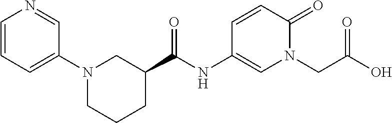 [SMILES: O=C(O)Cn1cc(NC(=O)[C@H]2CCCN(c3cccnc3)C2)ccc1=O] |
| I-92 | 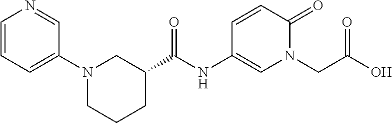 [SMILES: O=C(O)Cn1cc(NC(=O)[C@@H]2CCCN(c3cccnc3)C2)ccc1=O] |
| I-93 | 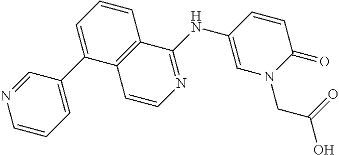 [SMILES: O=C(O)Cn1cc(Nc2nccc3c(-c4cccnc4)cccc23)ccc1=O] |
| I-94 | 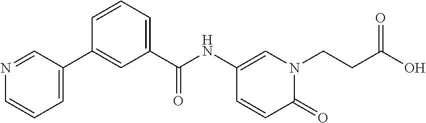 [SMILES: O=C(O)CCn1cc(NC(=O)c2cccc(-c3cccnc3)c2)ccc1=O] |
| I-95 | 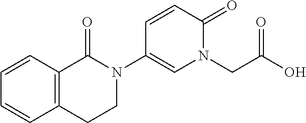 [SMILES: O=C(O)Cn1cc(N2CCc3ccccc3C2=O)ccc1=O] |
| I-96 | 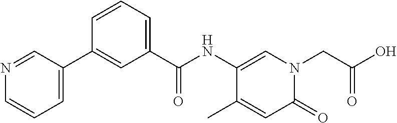 [SMILES: Cc1cc(=O)n(CC(=O)O)cc1NC(=O)c1cccc(-c2cccnc2)c1] |
| I-97 | 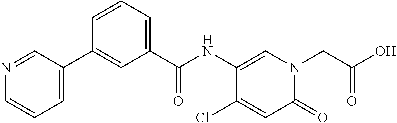 [SMILES: O=C(O)Cn1cc(NC(=O)c2cccc(-c3cccnc3)c2)c(Cl)cc1=O] |
| I-98 | 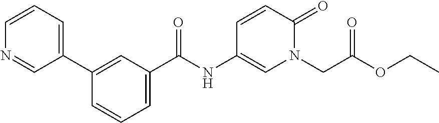 [SMILES: CCOC(=O)Cn1cc(NC(=O)c2cccc(-c3cccnc3)c2)ccc1=O] |
| I-99 | 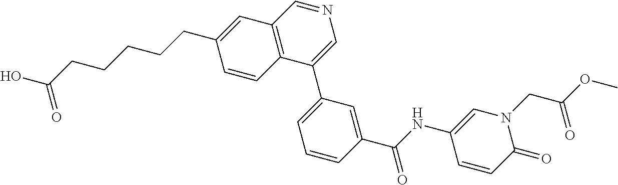 [SMILES: COC(=O)Cn1cc(NC(=O)c2cccc(-c3cncc4cc(CCCCCC(=O)O)ccc34)c2)ccc1=O] |
| I-100 | 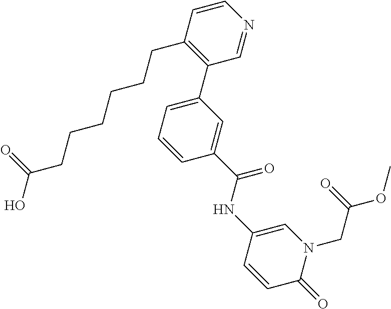 [SMILES: COC(=O)Cn1cc(NC(=O)c2cccc(-c3cnccc3CCCCCCC(=O)O)c2)ccc1=O] |
| I-101 | 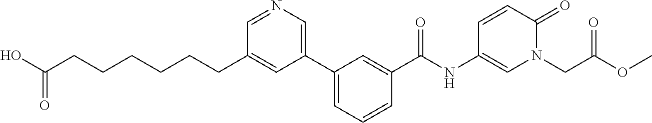 [SMILES: COC(=O)Cn1cc(NC(=O)c2cccc(-c3cncc(CCCCCCC(=O)O)c3)c2)ccc1=O] |
| I-102 | 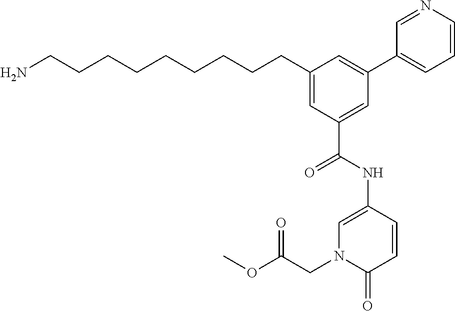 [SMILES: COC(=O)Cn1cc(NC(=O)c2cc(CCCCCCCCCN)cc(-c3cccnc3)c2)ccc1=O] |
| I-103 | 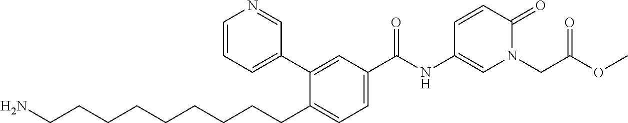 [SMILES: COC(=O)Cn1cc(NC(=O)c2ccc(CCCCCCCCCN)c(-c3cccnc3)c2)ccc1=O] |
| I-104 | 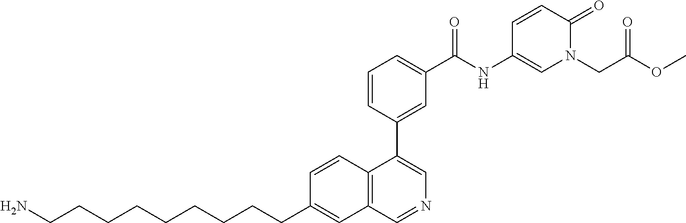 [SMILES: COC(=O)Cn1cc(NC(=O)c2cccc(-c3cncc4cc(CCCCCCCCCN)ccc34)c2)ccc1=O] |
| I-105 | 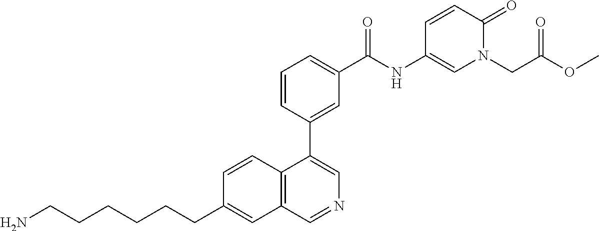 [SMILES: COC(=O)Cn1cc(NC(=O)c2cccc(-c3cncc4cc(CCCCCCN)ccc34)c2)ccc1=O] |
| I-106 | 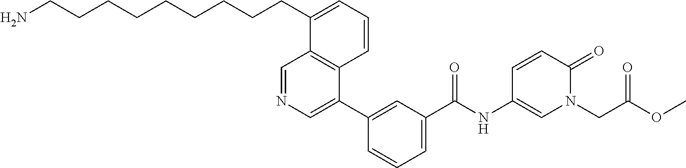 [SMILES: COC(=O)Cn1cc(NC(=O)c2cccc(-c3cncc4c(CCCCCCCCCN)cccc34)c2)ccc1=O] |
| I-107 | 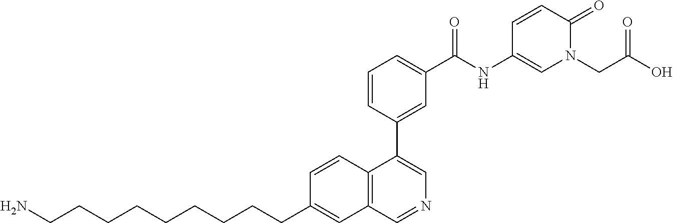 [SMILES: NCCCCCCCCCc1ccc2c(-c3cccc(C(=O)Nc4ccc(=O)n(CC(=O)O)c4)c3)cncc2c1] |
| I-108 | 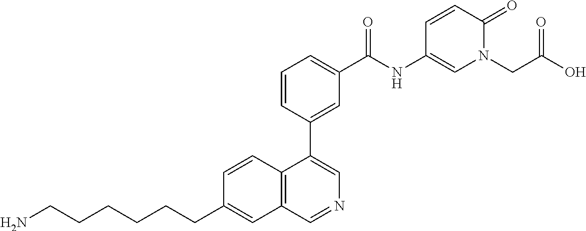 [SMILES: NCCCCCCc1ccc2c(-c3cccc(C(=O)Nc4ccc(=O)n(CC(=O)O)c4)c3)cncc2c1] |
| I-109 | 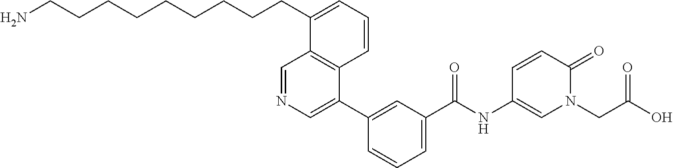 [SMILES: NCCCCCCCCCc1cccc2c(-c3cccc(C(=O)Nc4ccc(=O)n(CC(=O)O)c4)c3)cncc12] |
| I-110 | 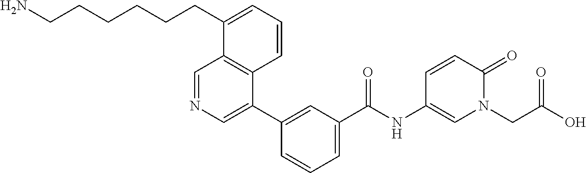 [SMILES: NCCCCCCc1cccc2c(-c3cccc(C(=O)Nc4ccc(=O)n(CC(=O)O)c4)c3)cncc12] |
| I-111 | 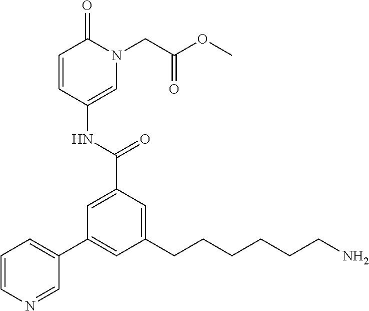 [SMILES: COC(=O)Cn1cc(NC(=O)c2cc(CCCCCCN)cc(-c3cccnc3)c2)ccc1=O] |
| I-112 | 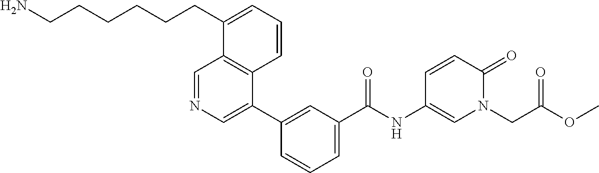 [SMILES: COC(=O)Cn1cc(NC(=O)c2cccc(-c3cncc4c(CCCCCCN)cccc34)c2)ccc1=O] |
| I-113 | 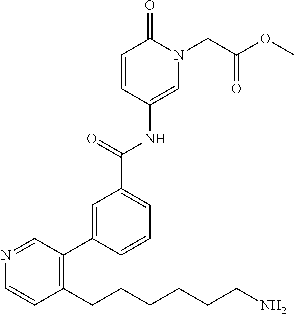 [SMILES: COC(=O)Cn1cc(NC(=O)c2cccc(-c3cnccc3CCCCCCN)c2)ccc1=O] |
| I-114 | 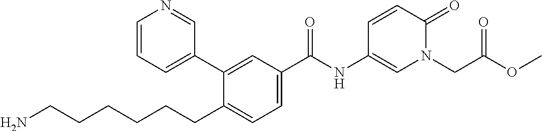 [SMILES: COC(=O)Cn1cc(NC(=O)c2ccc(CCCCCCN)c(-c3cccnc3)c2)ccc1=O] |
| I-115 | 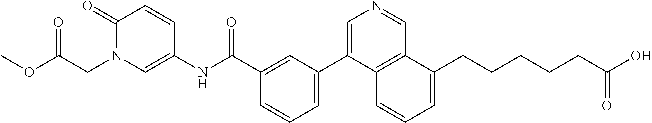 [SMILES: COC(=O)Cn1cc(NC(=O)c2cccc(-c3cncc4c(CCCCCC(=O)O)cccc34)c2)ccc1=O] |
| I-116 | 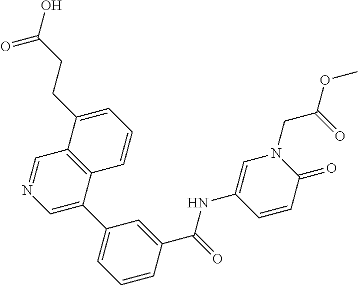 [SMILES: COC(=O)Cn1cc(NC(=O)c2cccc(-c3cncc4c(CCC(=O)O)cccc34)c2)ccc1=O] |
| I-117 | 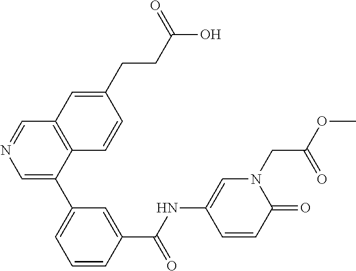 [SMILES: COC(=O)Cn1cc(NC(=O)c2cccc(-c3cncc4cc(CCC(=O)O)ccc34)c2)ccc1=O] |
| I-118 | 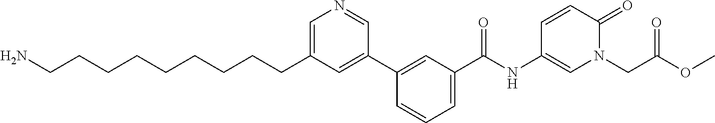 [SMILES: COC(=O)Cn1cc(NC(=O)c2cccc(-c3cncc(CCCCCCCCCN)c3)c2)ccc1=O] |
| I-119 | 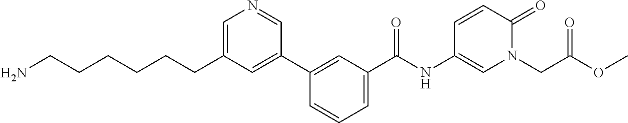 [SMILES: COC(=O)Cn1cc(NC(=O)c2cccc(-c3cncc(CCCCCCN)c3)c2)ccc1=O] |
| I-120 | 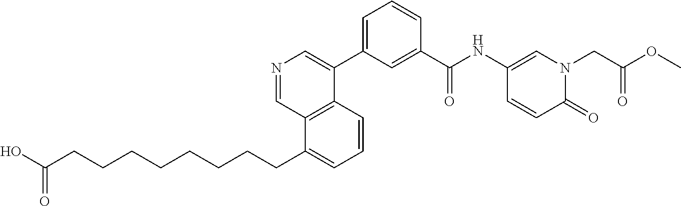 [SMILES: COC(=O)Cn1cc(NC(=O)c2cccc(-c3cncc4c(CCCCCCCCC(=O)O)cccc34)c2)ccc1=O] |
| I-121 | 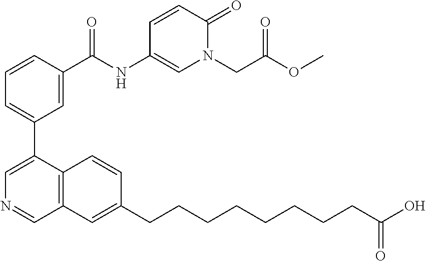 [SMILES: COC(=O)Cn1cc(NC(=O)c2cccc(-c3cncc4cc(CCCCCCCCC(=O)O)ccc34)c2)ccc1=O] |
| I-122 | 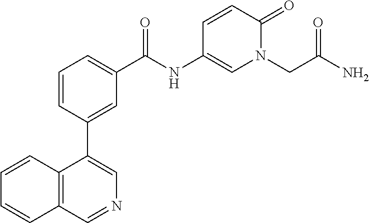 [SMILES: NC(=O)Cn1cc(NC(=O)c2cccc(-c3cncc4ccccc34)c2)ccc1=O] |
| I-123 | 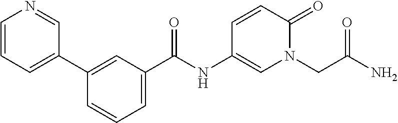 [SMILES: NC(=O)Cn1cc(NC(=O)c2cccc(-c3cccnc3)c2)ccc1=O] |
| I-124 | 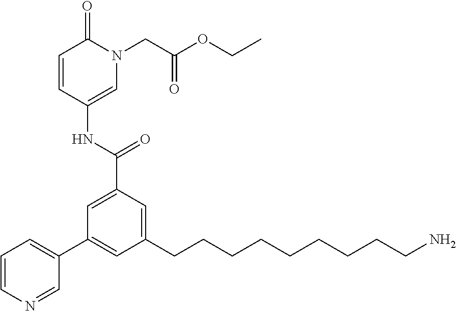 [SMILES: CCOC(=O)Cn1cc(NC(=O)c2cc(CCCCCCCCCN)cc(-c3cccnc3)c2)ccc1=O] |
| I-125 | 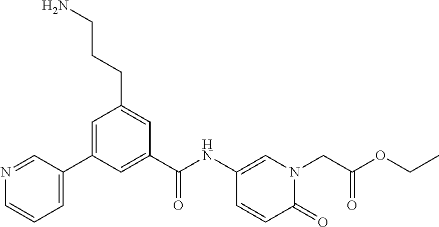 [SMILES: CCOC(=O)Cn1cc(NC(=O)c2cc(CCCN)cc(-c3cccnc3)c2)ccc1=O] |
| I-126 | 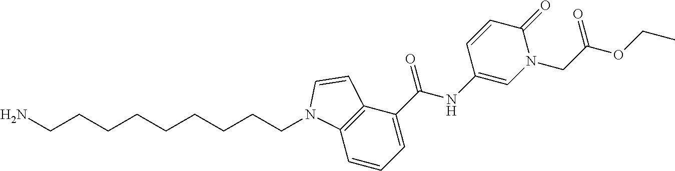 [SMILES: CCOC(=O)Cn1cc(NC(=O)c2cccc3c2ccn3CCCCCCCCCN)ccc1=O] |
| I-127 | 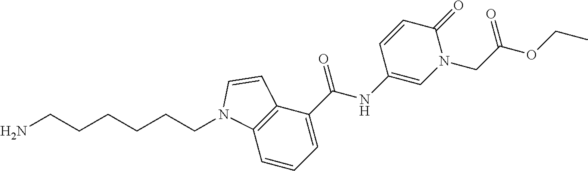 [SMILES: CCOC(=O)Cn1cc(NC(=O)c2cccc3c2ccn3CCCCCCN)ccc1=O] |
| I-128 | 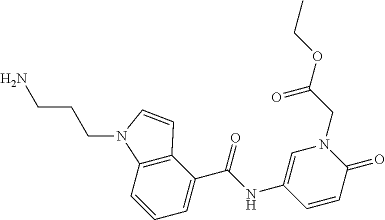 [SMILES: CCOC(=O)Cn1cc(NC(=O)c2cccc3c2ccn3CCCN)ccc1=O] |
| I-129 | 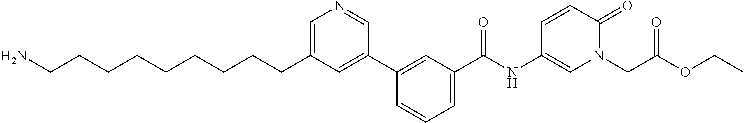 [SMILES: CCOC(=O)Cn1cc(NC(=O)c2cccc(-c3cncc(CCCCCCCCCN)c3)c2)ccc1=O] |
| I-130 | 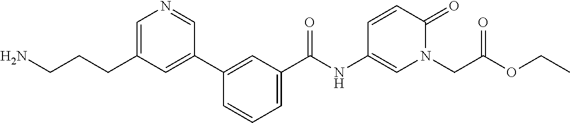 [SMILES: CCOC(=O)Cn1cc(NC(=O)c2cccc(-c3cncc(CCCN)c3)c2)ccc1=O] |
| I-131 | 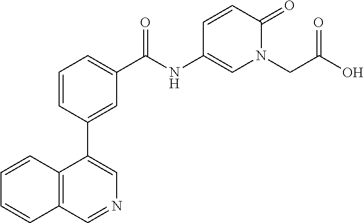 [SMILES: O=C(O)Cn1cc(NC(=O)c2cccc(-c3cncc4ccccc34)c2)ccc1=O] |
| I-132 | 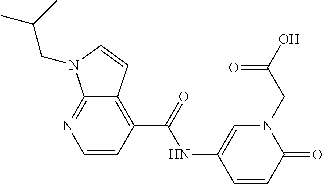 [SMILES: CC(C)Cn1ccc2c(C(=O)Nc3ccc(=O)n(CC(=O)O)c3)ccnc21] |
| I-133 | 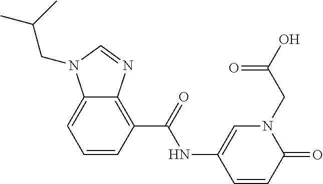 [SMILES: CC(C)Cn1cnc2c(C(=O)Nc3ccc(=O)n(CC(=O)O)c3)cccc21] |
| I-134 | 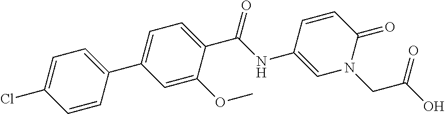 [SMILES: COc1cc(-c2ccc(Cl)cc2)ccc1C(=O)Nc1ccc(=O)n(CC(=O)O)c1] |
| I-135 | 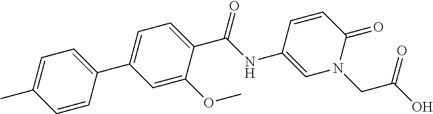 [SMILES: COc1cc(-c2ccc(C)cc2)ccc1C(=O)Nc1ccc(=O)n(CC(=O)O)c1] |
| I-136 | 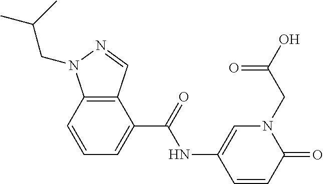 [SMILES: CC(C)Cn1ncc2c(C(=O)Nc3ccc(=O)n(CC(=O)O)c3)cccc21] |
| I-137 | 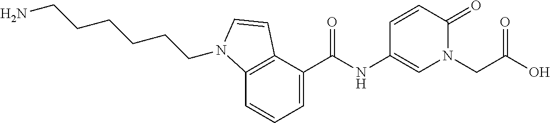 [SMILES: NCCCCCCn1ccc2c(C(=O)Nc3ccc(=O)n(CC(=O)O)c3)cccc21] |
| I-138 | 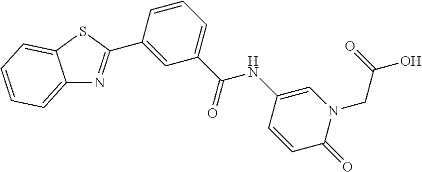 [SMILES: O=C(O)Cn1cc(NC(=O)c2cccc(-c3nc4ccccc4s3)c2)ccc1=O] |
| I-139 | 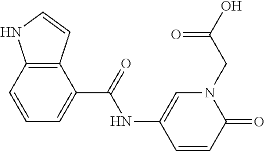 [SMILES: O=C(O)Cn1cc(NC(=O)c2cccc3[nH]ccc23)ccc1=O] |
| I-140 | 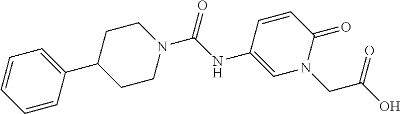 [SMILES: O=C(O)Cn1cc(NC(=O)N2CCC(c3ccccc3)CC2)ccc1=O] |
| I-141 | 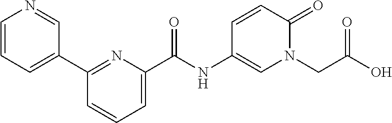 [SMILES: O=C(O)Cn1cc(NC(=O)c2cccc(-c3cccnc3)n2)ccc1=O] |
| I-142 | 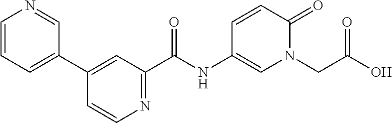 [SMILES: O=C(O)Cn1cc(NC(=O)c2cc(-c3cccnc3)ccn2)ccc1=O] |
| I-143 | 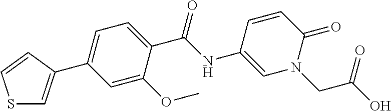 [SMILES: COc1cc(-c2ccsc2)ccc1C(=O)Nc1ccc(=O)n(CC(=O)O)c1] |
| I-144 | 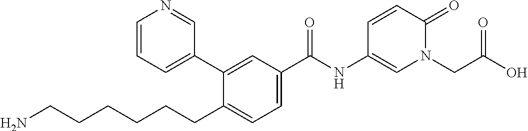 [SMILES: NCCCCCCc1ccc(C(=O)Nc2ccc(=O)n(CC(=O)O)c2)cc1-c1cccnc1] |
| I-145 | 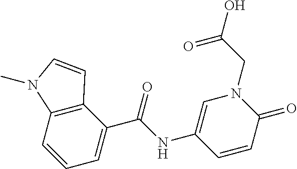 [SMILES: Cn1ccc2c(C(=O)Nc3ccc(=O)n(CC(=O)O)c3)cccc21] |
| I-146 | 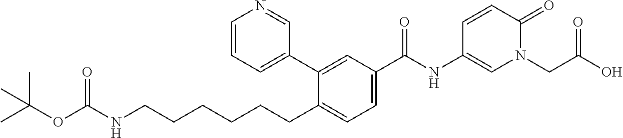 [SMILES: CC(C)(C)OC(=O)NCCCCCCc1ccc(C(=O)Nc2ccc(=O)n(CC(=O)O)c2)cc1-c1cccnc1] |
| I-147 | 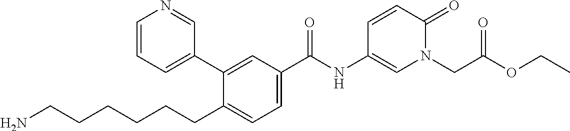 [SMILES: CCOC(=O)Cn1cc(NC(=O)c2ccc(CCCCCCN)c(-c3cccnc3)c2)ccc1=O] |
| I-148 | 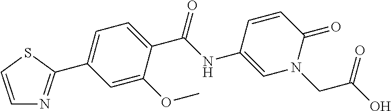 [SMILES: COc1cc(-c2nccs2)ccc1C(=O)Nc1ccc(=O)n(CC(=O)O)c1] |
| I-149 | 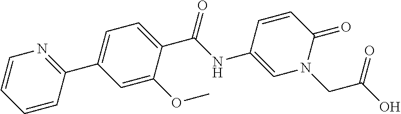 [SMILES: COc1cc(-c2ccccn2)ccc1C(=O)Nc1ccc(=O)n(CC(=O)O)c1] |
| I-150 | 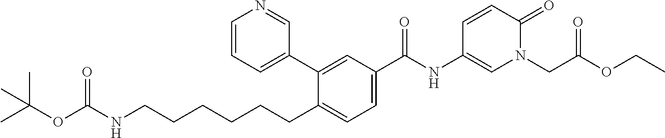 [SMILES: CCOC(=O)Cn1cc(NC(=O)c2ccc(CCCCCCNC(=O)OC(C)(C)C)c(-c3cccnc3)c2)ccc1=O] |
| I-151 | 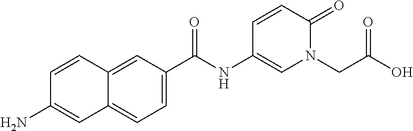 [SMILES: Nc1ccc2cc(C(=O)Nc3ccc(=O)n(CC(=O)O)c3)ccc2c1] |
| I-152 | 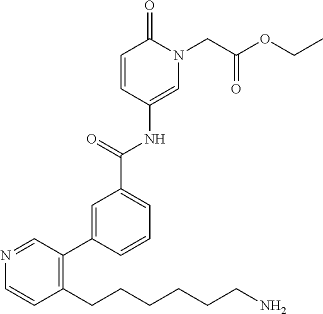 [SMILES: CCOC(=O)Cn1cc(NC(=O)c2cccc(-c3cnccc3CCCCCCN)c2)ccc1=O] |
| I-153 | 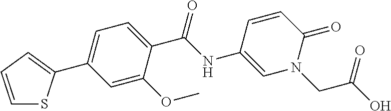 [SMILES: COc1cc(-c2cccs2)ccc1C(=O)Nc1ccc(=O)n(CC(=O)O)c1] |
| I-154 | 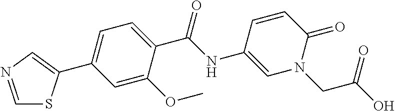 [SMILES: COc1cc(-c2cncs2)ccc1C(=O)Nc1ccc(=O)n(CC(=O)O)c1] |
| I-155 | 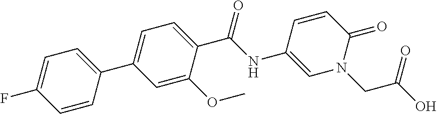 [SMILES: COc1cc(-c2ccc(F)cc2)ccc1C(=O)Nc1ccc(=O)n(CC(=O)O)c1] |
| I-156 | 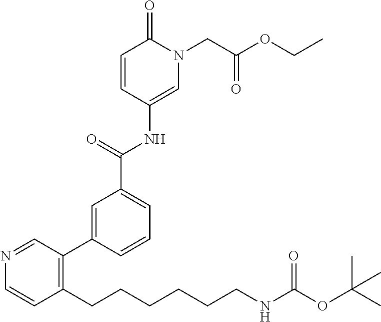 [SMILES: CCOC(=O)Cn1cc(NC(=O)c2cccc(-c3cnccc3CCCCCCNC(=O)OC(C)(C)C)c2)ccc1=O] |
| I-157 | 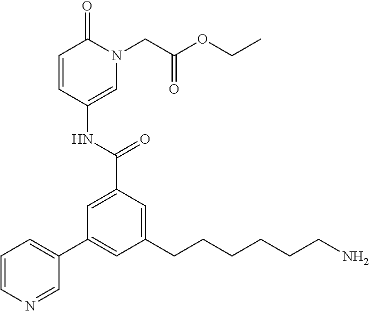 [SMILES: CCOC(=O)Cn1cc(NC(=O)c2cc(CCCCCCN)cc(-c3cccnc3)c2)ccc1=O] |
| I-158 | 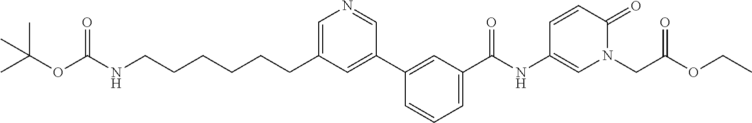 [SMILES: CCOC(=O)Cn1cc(NC(=O)c2cccc(-c3cncc(CCCCCCNC(=O)OC(C)(C)C)c3)c2)ccc1=O] |
| I-159 | 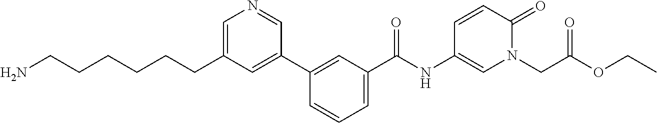 [SMILES: CCOC(=O)Cn1cc(NC(=O)c2cccc(-c3cncc(CCCCCCN)c3)c2)ccc1=O] |
| I-160 | 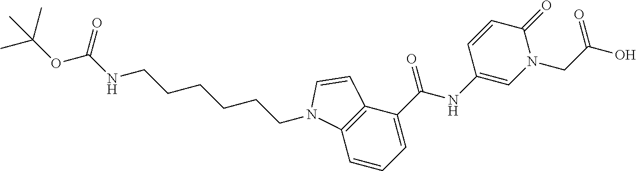 [SMILES: CC(C)(C)OC(=O)NCCCCCCn1ccc2c(C(=O)Nc3ccc(=O)n(CC(=O)O)c3)cccc21] |
| I-161 | 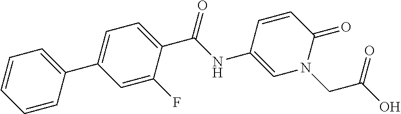 [SMILES: O=C(O)Cn1cc(NC(=O)c2ccc(-c3ccccc3)cc2F)ccc1=O] |
| I-162 | 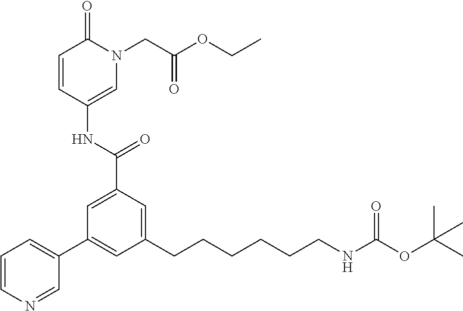 [SMILES: CCOC(=O)Cn1cc(NC(=O)c2cc(CCCCCCNC(=O)OC(C)(C)C)cc(-c3cccnc3)c2)ccc1=O] |
| I-163 | 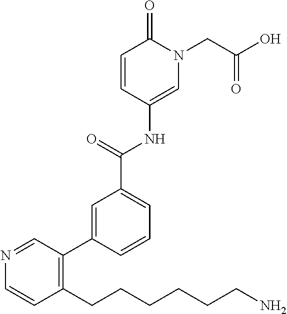 [SMILES: NCCCCCCc1ccncc1-c1cccc(C(=O)Nc2ccc(=O)n(CC(=O)O)c2)c1] |
| I-164 | 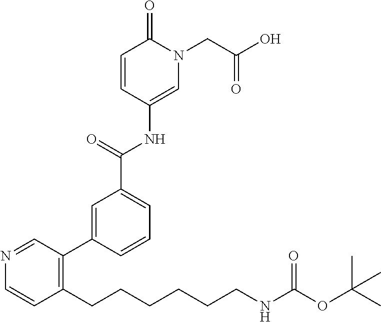 [SMILES: CC(C)(C)OC(=O)NCCCCCCc1ccncc1-c1cccc(C(=O)Nc2ccc(=O)n(CC(=O)O)c2)c1] |
| I-165 | 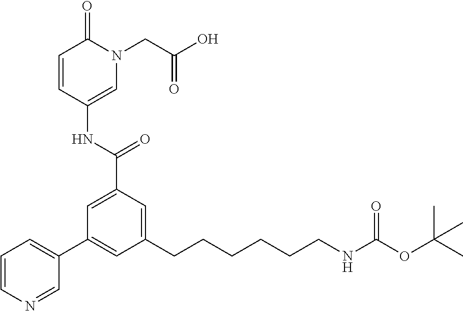 [SMILES: CC(C)(C)OC(=O)NCCCCCCc1cc(C(=O)Nc2ccc(=O)n(CC(=O)O)c2)cc(-c2cccnc2)c1] |
| I-166 | 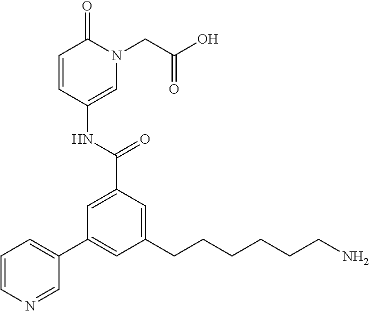 [SMILES: NCCCCCCc1cc(C(=O)Nc2ccc(=O)n(CC(=O)O)c2)cc(-c2cccnc2)c1] |
| I-167 | 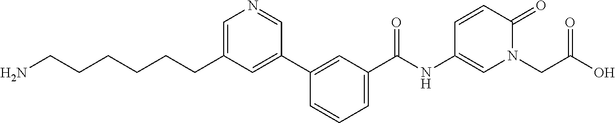 [SMILES: NCCCCCCc1cncc(-c2cccc(C(=O)Nc3ccc(=O)n(CC(=O)O)c3)c2)c1] |
| I-168 | 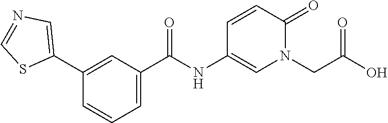 [SMILES: O=C(O)Cn1cc(NC(=O)c2cccc(-c3cncs3)c2)ccc1=O] |
| I-169 | 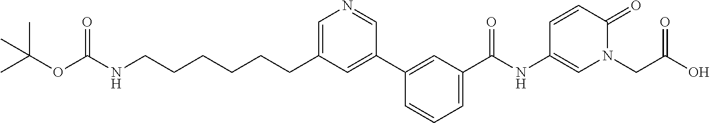 [SMILES: CC(C)(C)OC(=O)NCCCCCCc1cncc(-c2cccc(C(=O)Nc3ccc(=O)n(CC(=O)O)c3)c2)c1] |
| I-170 | 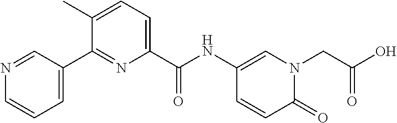 [SMILES: Cc1ccc(C(=O)Nc2ccc(=O)n(CC(=O)O)c2)nc1-c1cccnc1] |
| I-171 | 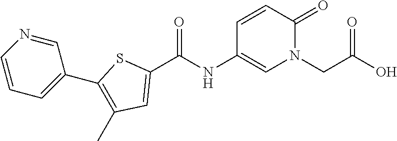 [SMILES: Cc1cc(C(=O)Nc2ccc(=O)n(CC(=O)O)c2)sc1-c1cccnc1] |
| I-172 | 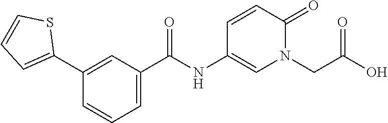 [SMILES: O=C(O)Cn1cc(NC(=O)c2cccc(-c3cccs3)c2)ccc1=O] |
| I-173 | 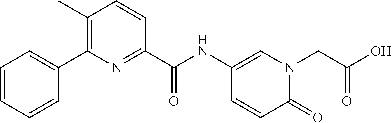 [SMILES: Cc1ccc(C(=O)Nc2ccc(=O)n(CC(=O)O)c2)nc1-c1ccccc1] |
| I-174 | 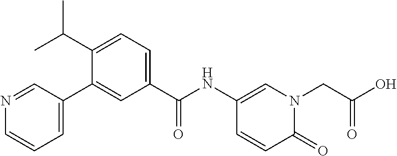 [SMILES: CC(C)c1ccc(C(=O)Nc2ccc(=O)n(CC(=O)O)c2)cc1-c1cccnc1] |
| I-175 | 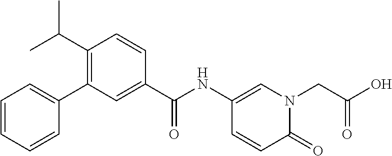 [SMILES: CC(C)c1ccc(C(=O)Nc2ccc(=O)n(CC(=O)O)c2)cc1-c1ccccc1] |
| I-176 | 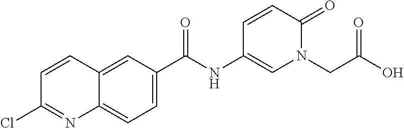 [SMILES: O=C(O)Cn1cc(NC(=O)c2ccc3nc(Cl)ccc3c2)ccc1=O] |
| I-177 | 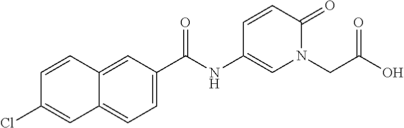 [SMILES: O=C(O)Cn1cc(NC(=O)c2ccc3cc(Cl)ccc3c2)ccc1=O] |
| I-178 | 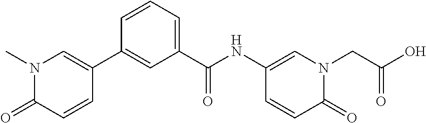 [SMILES: Cn1cc(-c2cccc(C(=O)Nc3ccc(=O)n(CC(=O)O)c3)c2)ccc1=O] |
| I-179 | 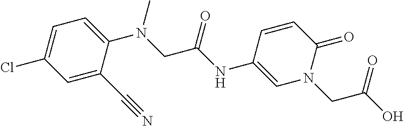 [SMILES: CN(CC(=O)Nc1ccc(=O)n(CC(=O)O)c1)c1ccc(Cl)cc1C#N] |
| I-180 | 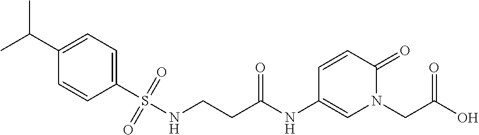 [SMILES: CC(C)c1ccc(S(=O)(=O)NCCC(=O)Nc2ccc(=O)n(CC(=O)O)c2)cc1] |
| I-181 | 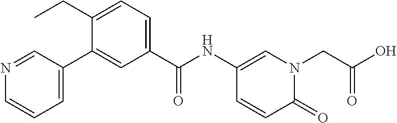 [SMILES: CCc1ccc(C(=O)Nc2ccc(=O)n(CC(=O)O)c2)cc1-c1cccnc1] |
| I-182 | 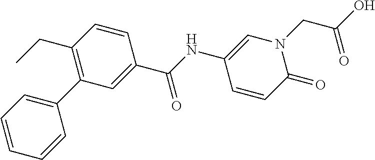 [SMILES: CCc1ccc(C(=O)Nc2ccc(=O)n(CC(=O)O)c2)cc1-c1ccccc1] |
| I-183 | 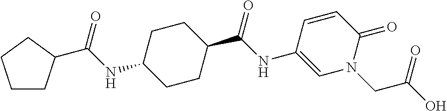 [SMILES: O=C(O)Cn1cc(NC(=O)[C@H]2CC[C@H](NC(=O)C3CCCC3)CC2)ccc1=O] |
| I-184 | 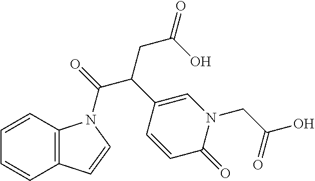 [SMILES: O=C(O)CC(C(=O)n1ccc2ccccc21)c1ccc(=O)n(CC(=O)O)c1] |
| I-185 | 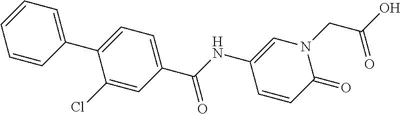 [SMILES: O=C(O)Cn1cc(NC(=O)c2ccc(-c3ccccc3)c(Cl)c2)ccc1=O] |
| I-186 | 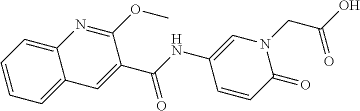 [SMILES: COc1nc2ccccc2cc1C(=O)Nc1ccc(=O)n(CC(=O)O)c1] |
| I-187 | 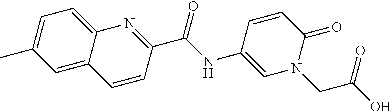 [SMILES: Cc1ccc2nc(C(=O)Nc3ccc(=O)n(CC(=O)O)c3)ccc2c1] |
| I-188 | 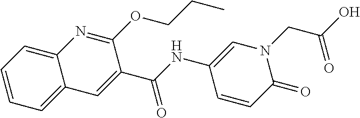 [SMILES: CCCOc1nc2ccccc2cc1C(=O)Nc1ccc(=O)n(CC(=O)O)c1] |
| I-189 | 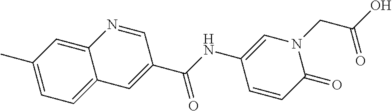 [SMILES: Cc1ccc2cc(C(=O)Nc3ccc(=O)n(CC(=O)O)c3)cnc2c1] |
| I-190 | 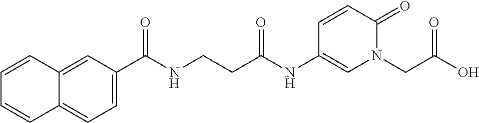 [SMILES: O=C(O)Cn1cc(NC(=O)CCNC(=O)c2ccc3ccccc3c2)ccc1=O] |
| I-191 | 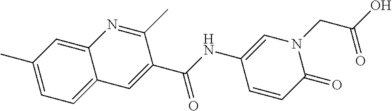 [SMILES: Cc1ccc2cc(C(=O)Nc3ccc(=O)n(CC(=O)O)c3)c(C)nc2c1] |
| I-192 | 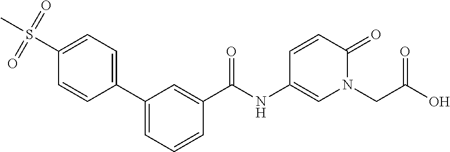 [SMILES: CS(=O)(=O)c1ccc(-c2cccc(C(=O)Nc3ccc(=O)n(CC(=O)O)c3)c2)cc1] |
| I-193 | 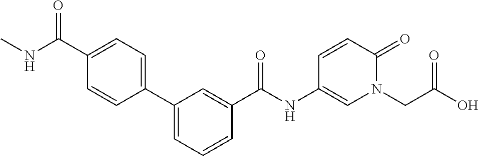 [SMILES: CNC(=O)c1ccc(-c2cccc(C(=O)Nc3ccc(=O)n(CC(=O)O)c3)c2)cc1] |
| I-194 | 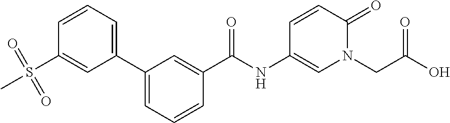 [SMILES: CS(=O)(=O)c1cccc(-c2cccc(C(=O)Nc3ccc(=O)n(CC(=O)O)c3)c2)c1] |
| I-195 | 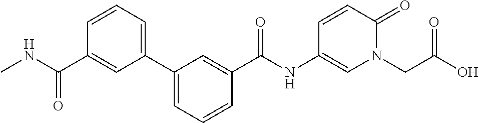 [SMILES: CNC(=O)c1cccc(-c2cccc(C(=O)Nc3ccc(=O)n(CC(=O)O)c3)c2)c1] |
| I-196 | 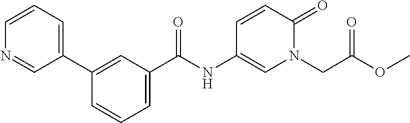 [SMILES: COC(=O)Cn1cc(NC(=O)c2cccc(-c3cccnc3)c2)ccc1=O] |
| I-197 | 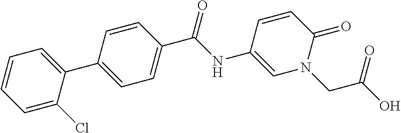 [SMILES: O=C(O)Cn1cc(NC(=O)c2ccc(-c3ccccc3Cl)cc2)ccc1=O] |
| I-198 | 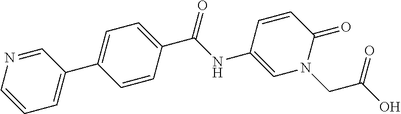 [SMILES: O=C(O)Cn1cc(NC(=O)c2ccc(-c3cccnc3)cc2)ccc1=O] |
| I-199 | 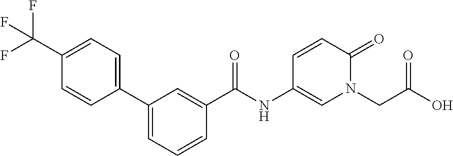 [SMILES: O=C(O)Cn1cc(NC(=O)c2cccc(-c3ccc(C(F)(F)F)cc3)c2)ccc1=O] |
| I-200 | 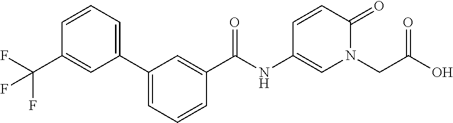 [SMILES: O=C(O)Cn1cc(NC(=O)c2cccc(-c3cccc(C(F)(F)F)c3)c2)ccc1=O] |
| I-201 | 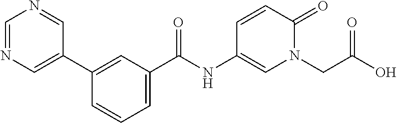 [SMILES: O=C(O)Cn1cc(NC(=O)c2cccc(-c3cncnc3)c2)ccc1=O] |
| I-202 | 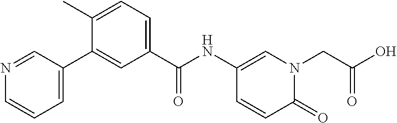 [SMILES: Cc1ccc(C(=O)Nc2ccc(=O)n(CC(=O)O)c2)cc1-c1cccnc1] |
| I-203 | 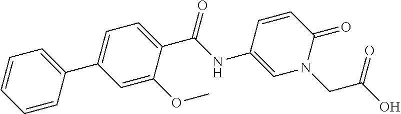 [SMILES: COc1cc(-c2ccccc2)ccc1C(=O)Nc1ccc(=O)n(CC(=O)O)c1] |
| I-204 | 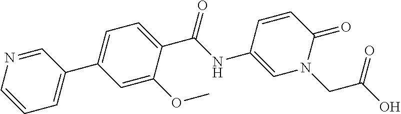 [SMILES: COc1cc(-c2cccnc2)ccc1C(=O)Nc1ccc(=O)n(CC(=O)O)c1] |
| I-205 | 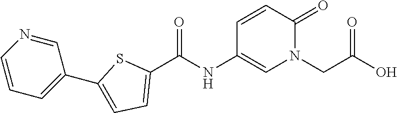 [SMILES: O=C(O)Cn1cc(NC(=O)c2ccc(-c3cccnc3)s2)ccc1=O] |
| I-206 | 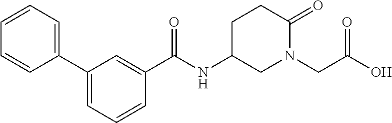 [SMILES: O=C(O)CN1CC(NC(=O)c2cccc(-c3ccccc3)c2)CCC1=O] |
| I-207 | 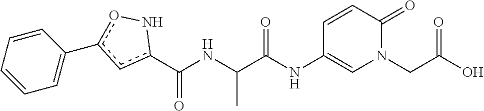 |
| I-208 | 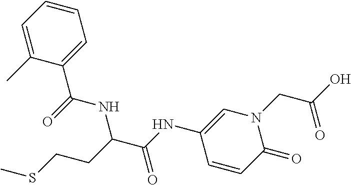 [SMILES: CSCCC(NC(=O)c1ccccc1C)C(=O)Nc1ccc(=O)n(CC(=O)O)c1] |
| I-209 | 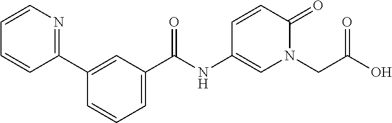 [SMILES: O=C(O)Cn1cc(NC(=O)c2cccc(-c3ccccn3)c2)ccc1=O] |
| I-210 | 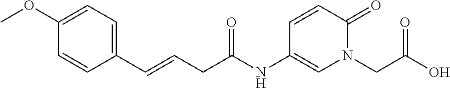 [SMILES: COc1ccc(/C=C/CC(=O)Nc2ccc(=O)n(CC(=O)O)c2)cc1] |
| I-211 | 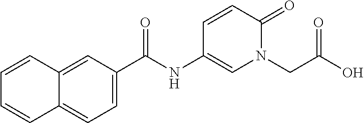 [SMILES: O=C(O)Cn1cc(NC(=O)c2ccc3ccccc3c2)ccc1=O] |
| I-212 | 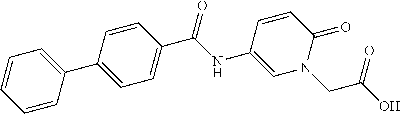 [SMILES: O=C(O)Cn1cc(NC(=O)c2ccc(-c3ccccc3)cc2)ccc1=O] |
| I-213 | 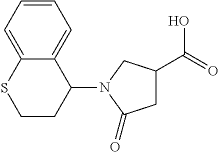 [SMILES: O=C(O)C1CC(=O)N(C2CCSc3ccccc32)C1] |
| I-214 | 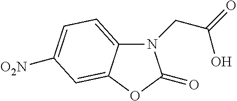 [SMILES: O=C(O)Cn1c(=O)oc2cc([N+](=O)[O-])ccc21] |
| I-215 | 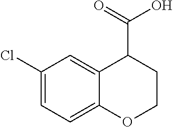 [SMILES: O=C(O)C1CCOc2ccc(Cl)cc21] |
| I-216 | 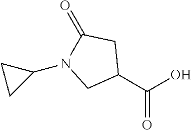 [SMILES: O=C(O)C1CC(=O)N(C2CC2)C1] |
| I-217 | 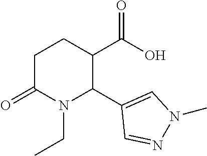 [SMILES: CCN1C(=O)CCC(C(=O)O)C1c1cnn(C)c1] |
| I-218 | 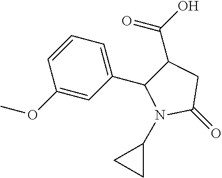 [SMILES: COc1cccc(C2C(C(=O)O)CC(=O)N2C2CC2)c1] |
| I-219 | 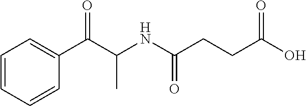 [SMILES: CC(NC(=O)CCC(=O)O)C(=O)c1ccccc1] |
| I-220 | 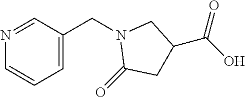 [SMILES: O=C(O)C1CC(=O)N(Cc2cccnc2)C1] |
| I-221 | 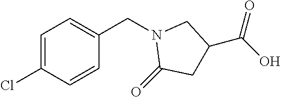 [SMILES: O=C(O)C1CC(=O)N(Cc2ccc(Cl)cc2)C1] |
| I-222 | 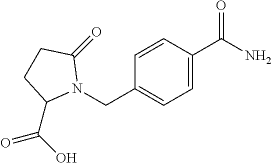 [SMILES: NC(=O)c1ccc(CN2C(=O)CCC2C(=O)O)cc1] |
| I-223 | 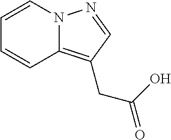 [SMILES: O=C(O)Cc1cnn2ccccc12] |
| I-224 | 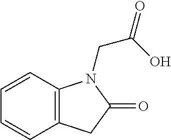 [SMILES: O=C(O)CN1C(=O)Cc2ccccc21] |
| I-225 | 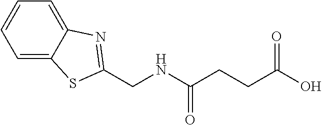 [SMILES: O=C(O)CCC(=O)NCc1nc2ccccc2s1] |
| I-226 | 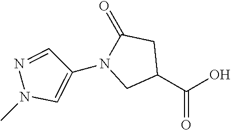 |
| I-227 | 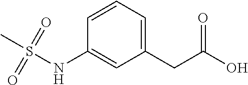 |
| I-228 | 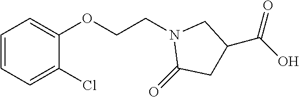 [SMILES: O=C(O)C1CC(=O)N(CCOc2ccccc2Cl)C1] |
| I-229 | 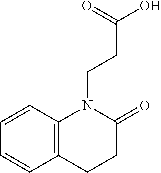 [SMILES: O=C(O)CCN1C(=O)CCc2ccccc21] |
| I-230 | 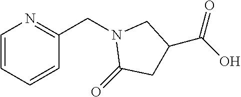 [SMILES: O=C(O)C1CC(=O)N(Cc2ccccn2)C1] |
| I-231 | 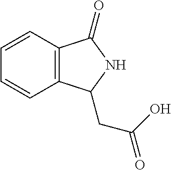 [SMILES: O=C(O)CC1NC(=O)c2ccccc21] |
| I-232 | 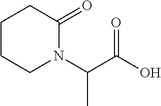 |
| I-233 | 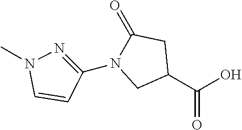 |
| I-234 | 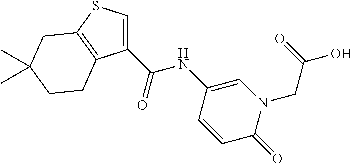 [SMILES: CC1(C)CCc2c(C(=O)Nc3ccc(=O)n(CC(=O)O)c3)csc2C1] |
| I-235 | 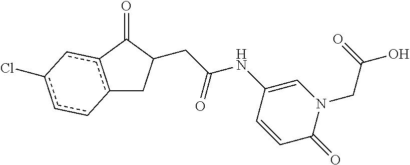 |
| I-236 | 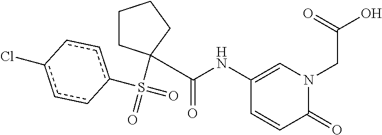 |
| I-237 | 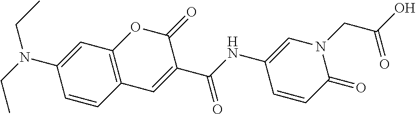 [SMILES: CCN(CC)c1ccc2cc(C(=O)Nc3ccc(=O)n(CC(=O)O)c3)c(=O)oc2c1] |
| I-238 | 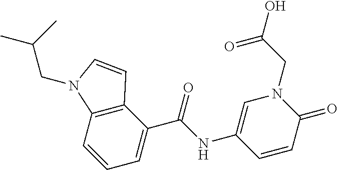 [SMILES: CC(C)Cn1ccc2c(C(=O)Nc3ccc(=O)n(CC(=O)O)c3)cccc21] |
| I-239 | 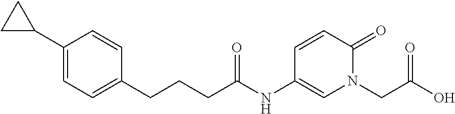 [SMILES: O=C(O)Cn1cc(NC(=O)CCCc2ccc(C3CC3)cc2)ccc1=O] |
| I-240 | 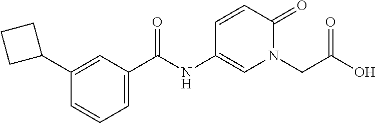 [SMILES: O=C(O)Cn1cc(NC(=O)c2cccc(C3CCC3)c2)ccc1=O] |
| I-241 | 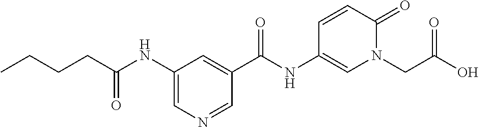 [SMILES: CCCCC(=O)Nc1cncc(C(=O)Nc2ccc(=O)n(CC(=O)O)c2)c1] |
| I-242 | 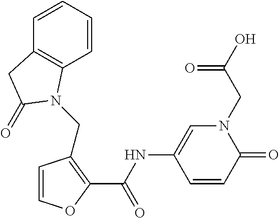 [SMILES: O=C(O)Cn1cc(NC(=O)c2occc2CN2C(=O)Cc3ccccc32)ccc1=O] |
| I-243 | 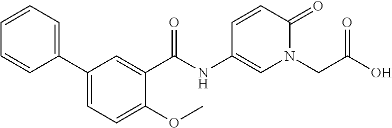 [SMILES: COc1ccc(-c2ccccc2)cc1C(=O)Nc1ccc(=O)n(CC(=O)O)c1] |
| I-244 | 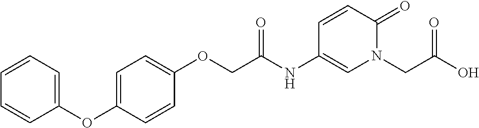 [SMILES: O=C(O)Cn1cc(NC(=O)COc2ccc(Oc3ccccc3)cc2)ccc1=O] |
| I-245 | 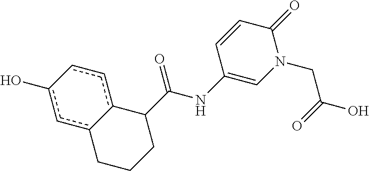 |
| I-246 | 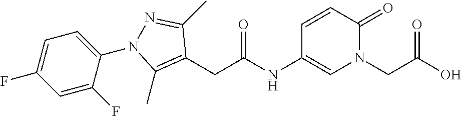 [SMILES: Cc1nn(-c2ccc(F)cc2F)c(C)c1CC(=O)Nc1ccc(=O)n(CC(=O)O)c1] |
| I-247 | 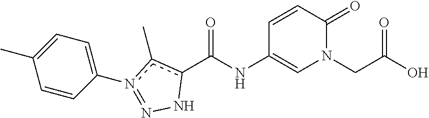 |
| I-248 | 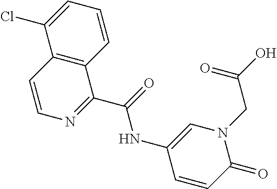 [SMILES: O=C(O)Cn1cc(NC(=O)c2nccc3c(Cl)cccc23)ccc1=O] |
| I-249 | 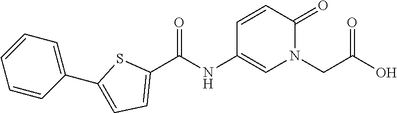 [SMILES: O=C(O)Cn1cc(NC(=O)c2ccc(-c3ccccc3)s2)ccc1=O] |
| I-250 | 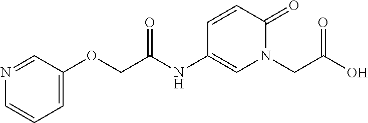 [SMILES: O=C(O)Cn1cc(NC(=O)COc2cccnc2)ccc1=O] |
| I-251 | 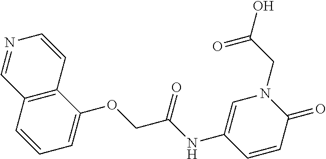 [SMILES: O=C(O)Cn1cc(NC(=O)COc2cccc3cnccc23)ccc1=O] |
| I-252 | 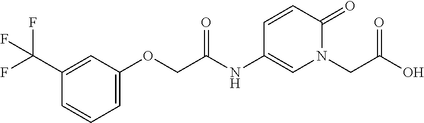 [SMILES: O=C(O)Cn1cc(NC(=O)COc2cccc(C(F)(F)F)c2)ccc1=O] |
| I-253 | 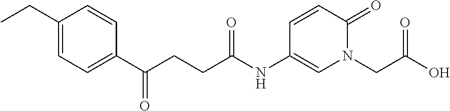 [SMILES: CCc1ccc(C(=O)CCC(=O)Nc2ccc(=O)n(CC(=O)O)c2)cc1] |
| I-254 | 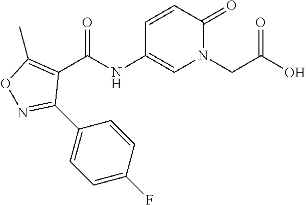 [SMILES: Cc1onc(-c2ccc(F)cc2)c1C(=O)Nc1ccc(=O)n(CC(=O)O)c1] |
| I-255 | 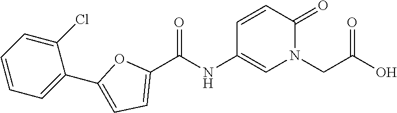 [SMILES: O=C(O)Cn1cc(NC(=O)c2ccc(-c3ccccc3Cl)o2)ccc1=O] |
| I-256 | 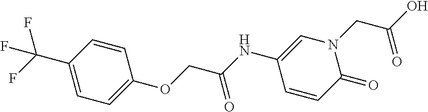 [SMILES: O=C(O)Cn1cc(NC(=O)COc2ccc(C(F)(F)F)cc2)ccc1=O] |
| I-257 | 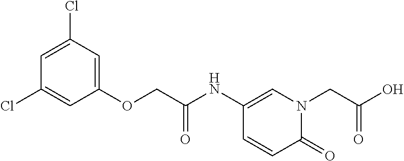 [SMILES: O=C(O)Cn1cc(NC(=O)COc2cc(Cl)cc(Cl)c2)ccc1=O] |
| I-258 | 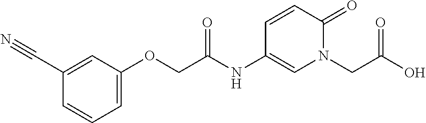 [SMILES: N#Cc1cccc(OCC(=O)Nc2ccc(=O)n(CC(=O)O)c2)c1] |
| I-259 | 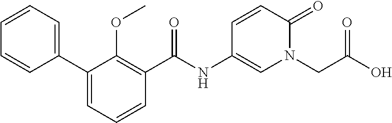 [SMILES: COc1c(C(=O)Nc2ccc(=O)n(CC(=O)O)c2)cccc1-c1ccccc1] |
| I-260 | 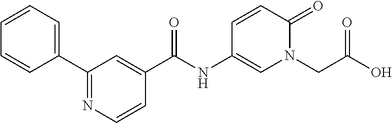 [SMILES: O=C(O)Cn1cc(NC(=O)c2ccnc(-c3ccccc3)c2)ccc1=O] |
| I-261 | 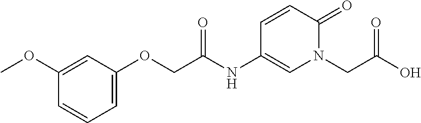 [SMILES: COc1cccc(OCC(=O)Nc2ccc(=O)n(CC(=O)O)c2)c1] |
| I-262 | 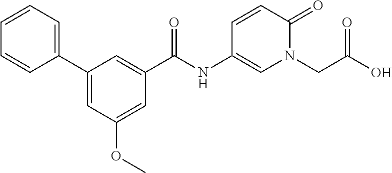 [SMILES: COc1cc(C(=O)Nc2ccc(=O)n(CC(=O)O)c2)cc(-c2ccccc2)c1] |
| I-263 | 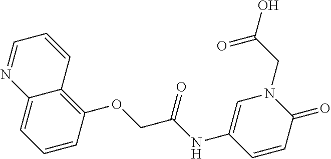 [SMILES: O=C(O)Cn1cc(NC(=O)COc2cccc3ncccc23)ccc1=O] |
| I-264 | 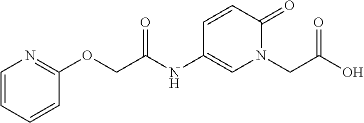 [SMILES: O=C(O)Cn1cc(NC(=O)COc2ccccn2)ccc1=O] |
| I-265 | 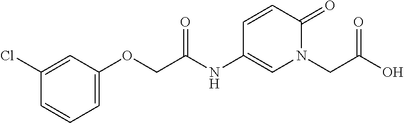 [SMILES: O=C(O)Cn1cc(NC(=O)COc2cccc(Cl)c2)ccc1=O] |
| I-266 | 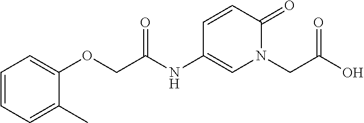 [SMILES: Cc1ccccc1OCC(=O)Nc1ccc(=O)n(CC(=O)O)c1] |
| I-267 | 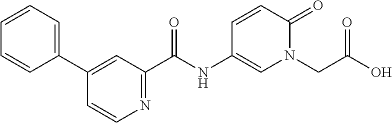 [SMILES: O=C(O)Cn1cc(NC(=O)c2cc(-c3ccccc3)ccn2)ccc1=O] |
| I-268 | 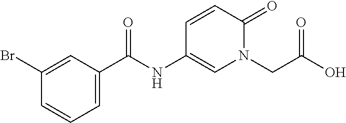 [SMILES: O=C(O)Cn1cc(NC(=O)c2cccc(Br)c2)ccc1=O] |
| I-269 | 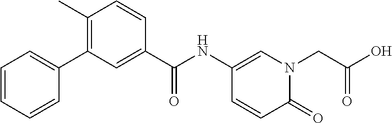 [SMILES: Cc1ccc(C(=O)Nc2ccc(=O)n(CC(=O)O)c2)cc1-c1ccccc1] |
| I-270 | 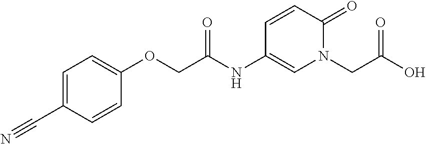 [SMILES: N#Cc1ccc(OCC(=O)Nc2ccc(=O)n(CC(=O)O)c2)cc1] |
| I-271 | 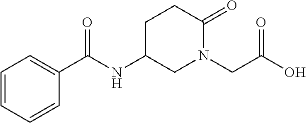 [SMILES: O=C(O)CN1CC(NC(=O)c2ccccc2)CCC1=O] |
| I-272 | 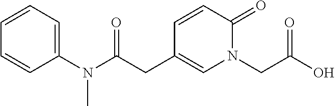 [SMILES: CN(C(=O)Cc1ccc(=O)n(CC(=O)O)c1)c1ccccc1] |
| I-273 | 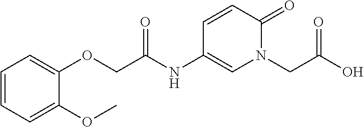 [SMILES: COc1ccccc1OCC(=O)Nc1ccc(=O)n(CC(=O)O)c1] |
| I-274 | 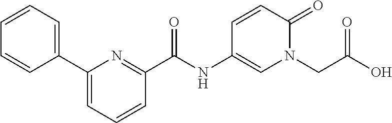 [SMILES: O=C(O)Cn1cc(NC(=O)c2cccc(-c3ccccc3)n2)ccc1=O] |
| I-275 | 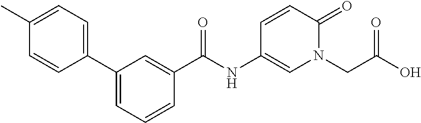 [SMILES: Cc1ccc(-c2cccc(C(=O)Nc3ccc(=O)n(CC(=O)O)c3)c2)cc1] |
| I-276 | 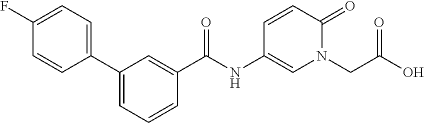 [SMILES: O=C(O)Cn1cc(NC(=O)c2cccc(-c3ccc(F)cc3)c2)ccc1=O] |
| I-277 | 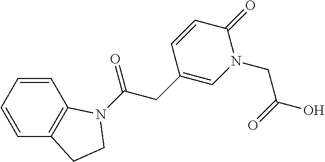 [SMILES: O=C(O)Cn1cc(CC(=O)N2CCc3ccccc32)ccc1=O] |
| I-278 | 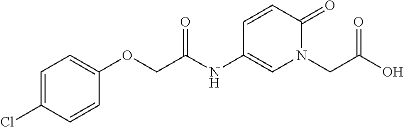 [SMILES: O=C(O)Cn1cc(NC(=O)COc2ccc(Cl)cc2)ccc1=O] |
| I-279 | 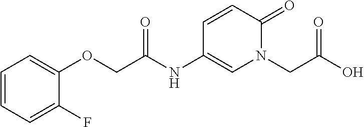 [SMILES: O=C(O)Cn1cc(NC(=O)COc2ccccc2F)ccc1=O] |
| I-280 | 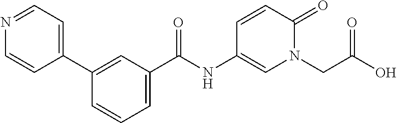 [SMILES: O=C(O)Cn1cc(NC(=O)c2cccc(-c3ccncc3)c2)ccc1=O] |
| I-281 | 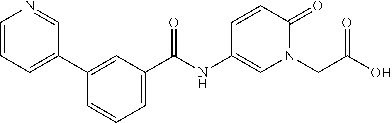 [SMILES: O=C(O)Cn1cc(NC(=O)c2cccc(-c3cccnc3)c2)ccc1=O] |
| I-282 | 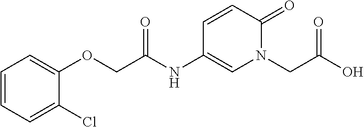 [SMILES: O=C(O)Cn1cc(NC(=O)COc2ccccc2Cl)ccc1=O] |
| I-283 | 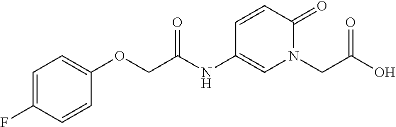 [SMILES: O=C(O)Cn1cc(NC(=O)COc2ccc(F)cc2)ccc1=O] |
| I-284 | 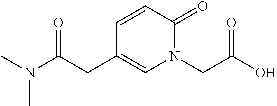 [SMILES: CN(C)C(=O)Cc1ccc(=O)n(CC(=O)O)c1] |
| I-285 | 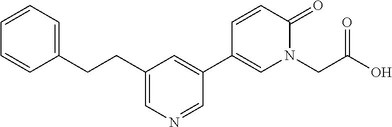 [SMILES: O=C(O)Cn1cc(-c2cncc(CCc3ccccc3)c2)ccc1=O] |
| I-286 | 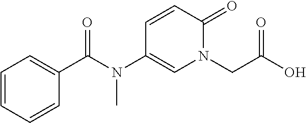 [SMILES: CN(C(=O)c1ccccc1)c1ccc(=O)n(CC(=O)O)c1] |
| I-287 | 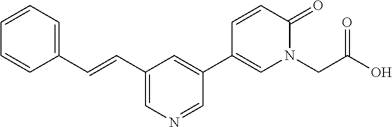 [SMILES: O=C(O)Cn1cc(-c2cncc(/C=C/c3ccccc3)c2)ccc1=O] |
| I-288 | 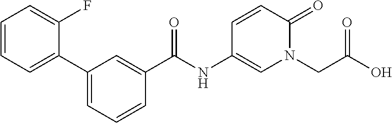 [SMILES: O=C(O)Cn1cc(NC(=O)c2cccc(-c3ccccc3F)c2)ccc1=O] |
| I-289 | 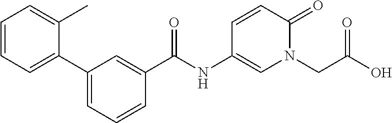 [SMILES: Cc1ccccc1-c1cccc(C(=O)Nc2ccc(=O)n(CC(=O)O)c2)c1] |
| I-290 | 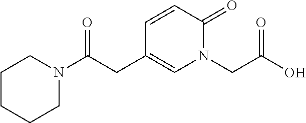 [SMILES: O=C(O)Cn1cc(CC(=O)N2CCCCC2)ccc1=O] |
| I-291 | 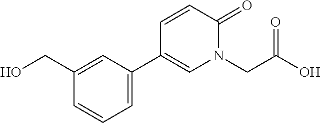 [SMILES: O=C(O)Cn1cc(-c2cccc(CO)c2)ccc1=O] |
| I-292 | 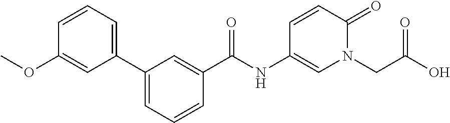 [SMILES: COc1cccc(-c2cccc(C(=O)Nc3ccc(=O)n(CC(=O)O)c3)c2)c1] |
| I-293 | 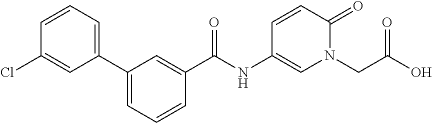 [SMILES: O=C(O)Cn1cc(NC(=O)c2cccc(-c3cccc(Cl)c3)c2)ccc1=O] |
| I-294 | 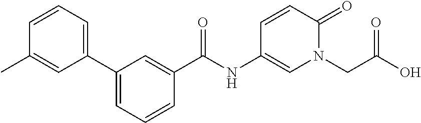 [SMILES: Cc1cccc(-c2cccc(C(=O)Nc3ccc(=O)n(CC(=O)O)c3)c2)c1] |
| I-295 | 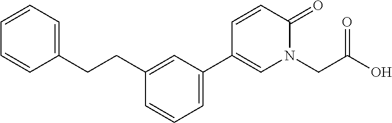 [SMILES: O=C(O)Cn1cc(-c2cccc(CCc3ccccc3)c2)ccc1=O] |
| I-296 | 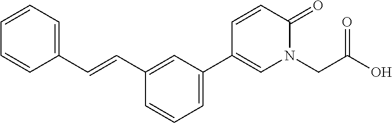 [SMILES: O=C(O)Cn1cc(-c2cccc(/C=C/c3ccccc3)c2)ccc1=O] |
| I-297 | 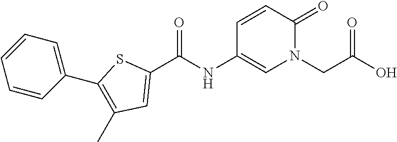 [SMILES: Cc1cc(C(=O)Nc2ccc(=O)n(CC(=O)O)c2)sc1-c1ccccc1] |
| I-298 | 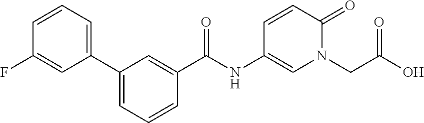 [SMILES: O=C(O)Cn1cc(NC(=O)c2cccc(-c3cccc(F)c3)c2)ccc1=O] |
| I-299 | 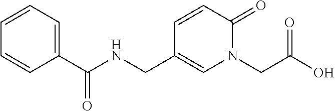 [SMILES: O=C(O)Cn1cc(CNC(=O)c2ccccc2)ccc1=O] |
| I-300 | 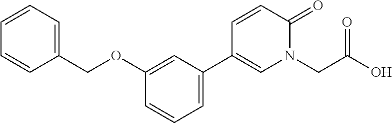 [SMILES: O=C(O)Cn1cc(-c2cccc(OCc3ccccc3)c2)ccc1=O] |
| I-301 | 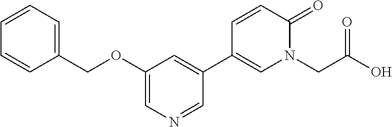 [SMILES: O=C(O)Cn1cc(-c2cncc(OCc3ccccc3)c2)ccc1=O] |
| I-302 | 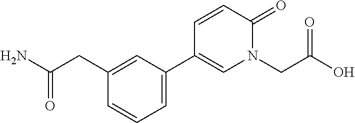 [SMILES: NC(=O)Cc1cccc(-c2ccc(=O)n(CC(=O)O)c2)c1] |
| I-303 | 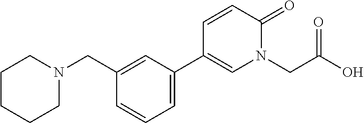 [SMILES: O=C(O)Cn1cc(-c2cccc(CN3CCCCC3)c2)ccc1=O] |
| I-304 | 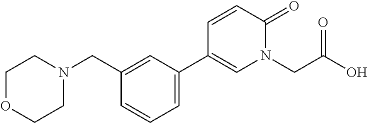 [SMILES: O=C(O)Cn1cc(-c2cccc(CN3CCOCC3)c2)ccc1=O] |
| I-305 | 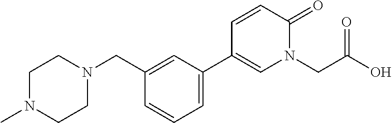 [SMILES: CN1CCN(Cc2cccc(-c3ccc(=O)n(CC(=O)O)c3)c2)CC1] |
| I-306 | 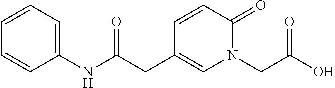 [SMILES: O=C(O)Cn1cc(CC(=O)Nc2ccccc2)ccc1=O] |
| I-307 | 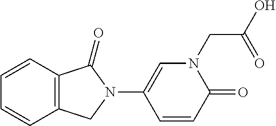 [SMILES: O=C(O)Cn1cc(N2Cc3ccccc3C2=O)ccc1=O] |
| I-308 | 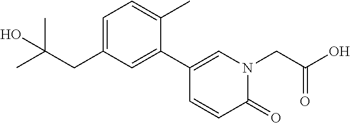 [SMILES: Cc1ccc(CC(C)(C)O)cc1-c1ccc(=O)n(CC(=O)O)c1] |
| I-309 | 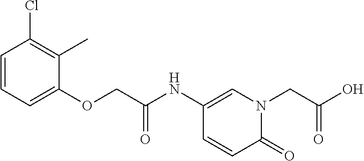 [SMILES: Cc1c(Cl)cccc1OCC(=O)Nc1ccc(=O)n(CC(=O)O)c1] |
| I-310 | 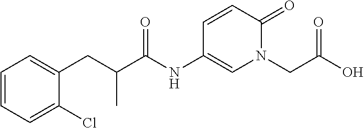 [SMILES: CC(Cc1ccccc1Cl)C(=O)Nc1ccc(=O)n(CC(=O)O)c1] |
| I-311 | 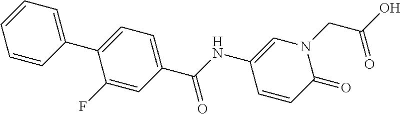 [SMILES: O=C(O)Cn1cc(NC(=O)c2ccc(-c3ccccc3)c(F)c2)ccc1=O] |
| I-312 | 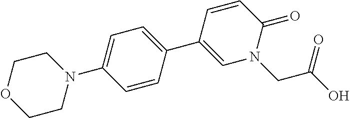 [SMILES: O=C(O)Cn1cc(-c2ccc(N3CCOCC3)cc2)ccc1=O] |
| I-313 | 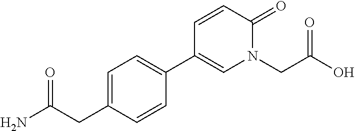 [SMILES: NC(=O)Cc1ccc(-c2ccc(=O)n(CC(=O)O)c2)cc1] |
| I-314 | 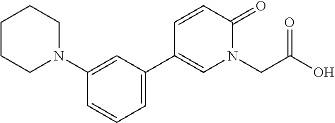 [SMILES: O=C(O)Cn1cc(-c2cccc(N3CCCCC3)c2)ccc1=O] |
| I-315 | 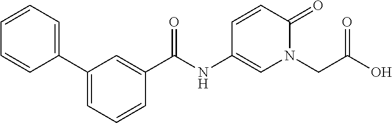 [SMILES: O=C(O)Cn1cc(NC(=O)c2cccc(-c3ccccc3)c2)ccc1=O] |
| I-316 | 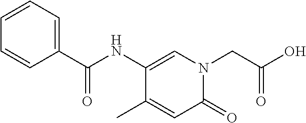 [SMILES: Cc1cc(=O)n(CC(=O)O)cc1NC(=O)c1ccccc1] |
| I-317 | 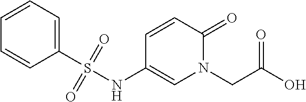 [SMILES: O=C(O)Cn1cc(NS(=O)(=O)c2ccccc2)ccc1=O] |
| I-318 | 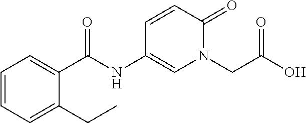 [SMILES: CCc1ccccc1C(=O)Nc1ccc(=O)n(CC(=O)O)c1] |
| I-319 | 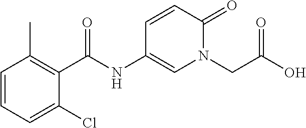 [SMILES: Cc1cccc(Cl)c1C(=O)Nc1ccc(=O)n(CC(=O)O)c1] |
| I-320 | 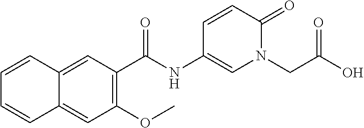 [SMILES: COc1cc2ccccc2cc1C(=O)Nc1ccc(=O)n(CC(=O)O)c1] |
| I-321 | 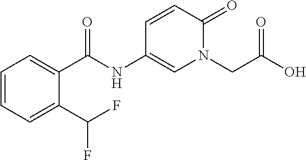 [SMILES: O=C(O)Cn1cc(NC(=O)c2ccccc2C(F)F)ccc1=O] |
| I-322 | 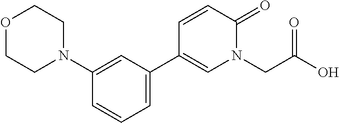 [SMILES: O=C(O)Cn1cc(-c2cccc(N3CCOCC3)c2)ccc1=O] |
| I-323 | 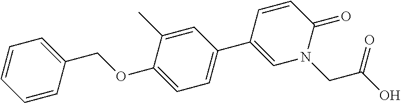 [SMILES: Cc1cc(-c2ccc(=O)n(CC(=O)O)c2)ccc1OCc1ccccc1] |
| I-324 | 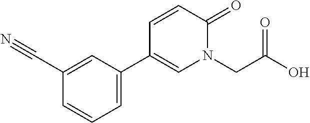 [SMILES: N#Cc1cccc(-c2ccc(=O)n(CC(=O)O)c2)c1] |
| I-325 | 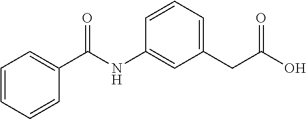 [SMILES: O=C(O)Cc1cccc(NC(=O)c2ccccc2)c1] |
| I-326 | 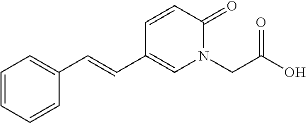 [SMILES: O=C(O)Cn1cc(/C=C/c2ccccc2)ccc1=O] |
| I-327 | 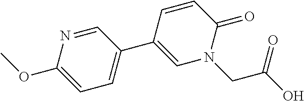 [SMILES: COc1ccc(-c2ccc(=O)n(CC(=O)O)c2)cn1] |
| I-328 | 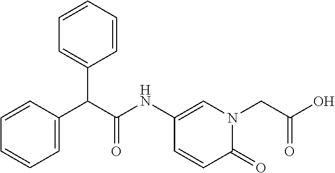 [SMILES: O=C(O)Cn1cc(NC(=O)C(c2ccccc2)c2ccccc2)ccc1=O] |
| I-329 | 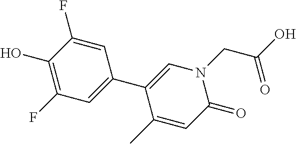 [SMILES: Cc1cc(=O)n(CC(=O)O)cc1-c1cc(F)c(O)c(F)c1] |
| I-330 | 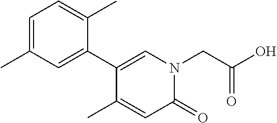 [SMILES: Cc1ccc(C)c(-c2cn(CC(=O)O)c(=O)cc2C)c1] |
| I-331 | 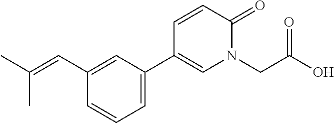 [SMILES: CC(C)=Cc1cccc(-c2ccc(=O)n(CC(=O)O)c2)c1] |
| I-332 | 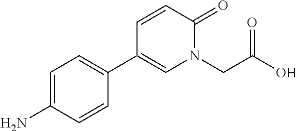 [SMILES: Nc1ccc(-c2ccc(=O)n(CC(=O)O)c2)cc1] |
| I-333 | 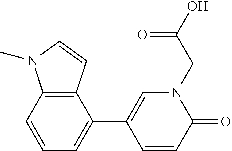 [SMILES: Cn1ccc2c(-c3ccc(=O)n(CC(=O)O)c3)cccc21] |
| I-334 | 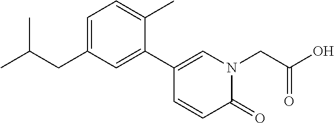 [SMILES: Cc1ccc(CC(C)C)cc1-c1ccc(=O)n(CC(=O)O)c1] |
| I-335 | 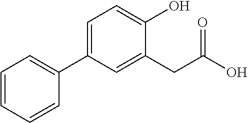 [SMILES: O=C(O)Cc1cc(-c2ccccc2)ccc1O] |
| I-336 | 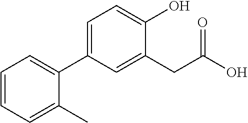 [SMILES: Cc1ccccc1-c1ccc(O)c(CC(=O)O)c1] |
| I-337 | 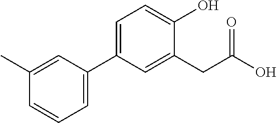 [SMILES: Cc1cccc(-c2ccc(O)c(CC(=O)O)c2)c1] |
| I-338 | 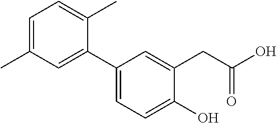 [SMILES: Cc1ccc(C)c(-c2ccc(O)c(CC(=O)O)c2)c1] |
| I-339 | 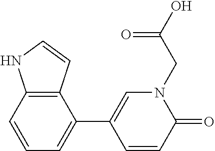 [SMILES: O=C(O)Cn1cc(-c2cccc3[nH]ccc23)ccc1=O] |
| I-340 | 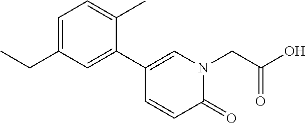 [SMILES: CCc1ccc(C)c(-c2ccc(=O)n(CC(=O)O)c2)c1] |
| I-341 | 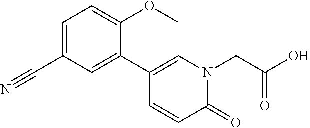 [SMILES: COc1ccc(C#N)cc1-c1ccc(=O)n(CC(=O)O)c1] |
| I-342 | 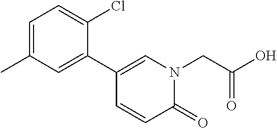 [SMILES: Cc1ccc(Cl)c(-c2ccc(=O)n(CC(=O)O)c2)c1] |
| I-343 | 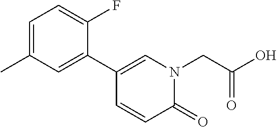 [SMILES: Cc1ccc(F)c(-c2ccc(=O)n(CC(=O)O)c2)c1] |
| I-344 | 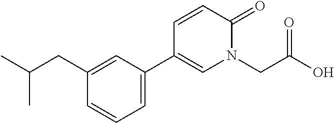 [SMILES: CC(C)Cc1cccc(-c2ccc(=O)n(CC(=O)O)c2)c1] |
| I-345 | 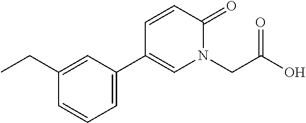 [SMILES: CCc1cccc(-c2ccc(=O)n(CC(=O)O)c2)c1] |
| I-346 | 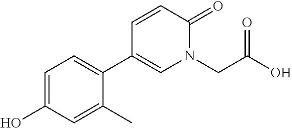 [SMILES: Cc1cc(O)ccc1-c1ccc(=O)n(CC(=O)O)c1] |
| I-347 | 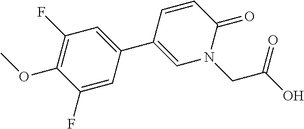 [SMILES: COc1c(F)cc(-c2ccc(=O)n(CC(=O)O)c2)cc1F] |
| I-348 | 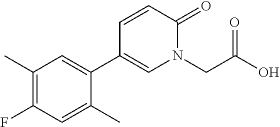 [SMILES: Cc1cc(-c2ccc(=O)n(CC(=O)O)c2)c(C)cc1F] |
| I-349 | 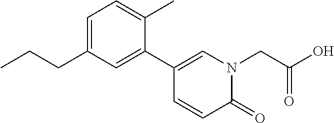 [SMILES: CCCc1ccc(C)c(-c2ccc(=O)n(CC(=O)O)c2)c1] |
| I-350 | 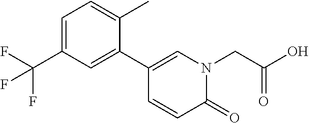 [SMILES: Cc1ccc(C(F)(F)F)cc1-c1ccc(=O)n(CC(=O)O)c1] |
| I-351 | 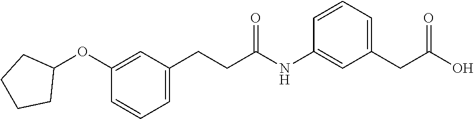 [SMILES: O=C(O)Cc1cccc(NC(=O)CCc2cccc(OC3CCCC3)c2)c1] |
| I-352 | 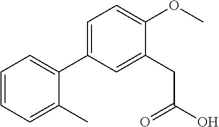 [SMILES: COc1ccc(-c2ccccc2C)cc1CC(=O)O] |
| I-353 | 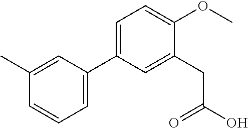 |
| I-354 | 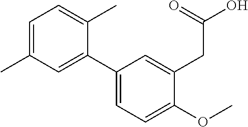 |
| I-355 | 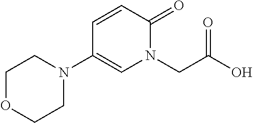 [SMILES: O=C(O)Cn1cc(N2CCOCC2)ccc1=O] |
| I-356 | 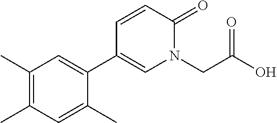 [SMILES: Cc1cc(C)c(-c2ccc(=O)n(CC(=O)O)c2)cc1C] |
| I-357 | 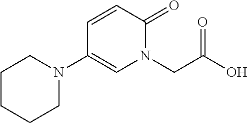 [SMILES: O=C(O)Cn1cc(N2CCCCC2)ccc1=O] |
| I-358 | 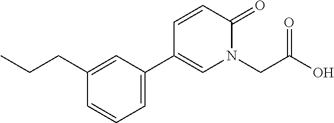 [SMILES: CCCc1cccc(-c2ccc(=O)n(CC(=O)O)c2)c1] |
| I-359 | 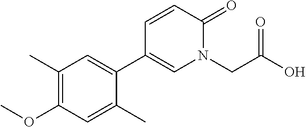 [SMILES: COc1cc(C)c(-c2ccc(=O)n(CC(=O)O)c2)cc1C] |
| I-360 | 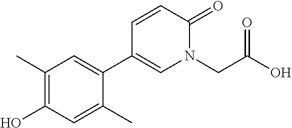 [SMILES: Cc1cc(-c2ccc(=O)n(CC(=O)O)c2)c(C)cc1O] |
| I-361 | 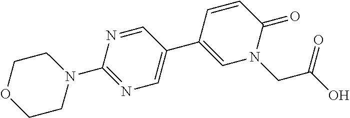 [SMILES: O=C(O)Cn1cc(-c2cnc(N3CCOCC3)nc2)ccc1=O] |
| I-362 | 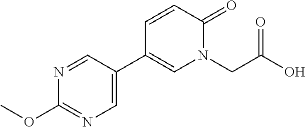 [SMILES: COc1ncc(-c2ccc(=O)n(CC(=O)O)c2)cn1] |
| I-363 | 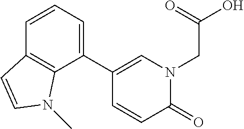 [SMILES: Cn1ccc2cccc(-c3ccc(=O)n(CC(=O)O)c3)c21] |
| I-364 | 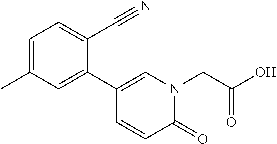 [SMILES: Cc1ccc(C#N)c(-c2ccc(=O)n(CC(=O)O)c2)c1] |
| I-365 | 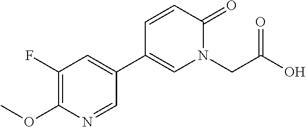 [SMILES: COc1ncc(-c2ccc(=O)n(CC(=O)O)c2)cc1F] |
| I-366 | 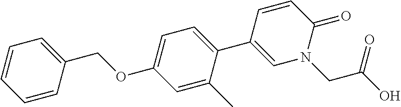 [SMILES: Cc1cc(OCc2ccccc2)ccc1-c1ccc(=O)n(CC(=O)O)c1] |
| I-367 | 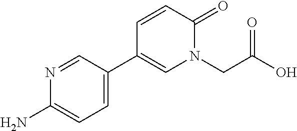 [SMILES: Nc1ccc(-c2ccc(=O)n(CC(=O)O)c2)cn1] |
| I-368 | 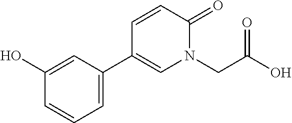 [SMILES: O=C(O)Cn1cc(-c2cccc(O)c2)ccc1=O] |
| I-369 | 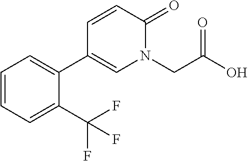 [SMILES: O=C(O)Cn1cc(-c2ccccc2C(F)(F)F)ccc1=O] |
| I-370 | 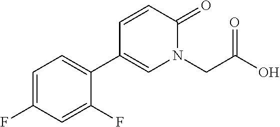 [SMILES: O=C(O)Cn1cc(-c2ccc(F)cc2F)ccc1=O] |
| I-371 | 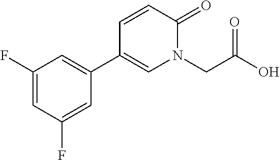 [SMILES: O=C(O)Cn1cc(-c2cc(F)cc(F)c2)ccc1=O] |
| I-372 | 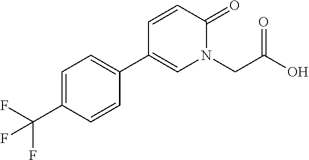 [SMILES: O=C(O)Cn1cc(-c2ccc(C(F)(F)F)cc2)ccc1=O] |
| I-373 | 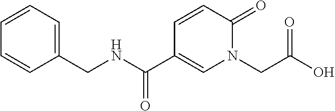 [SMILES: O=C(O)Cn1cc(C(=O)NCc2ccccc2)ccc1=O] |
| I-374 | 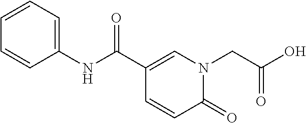 [SMILES: O=C(O)Cn1cc(C(=O)Nc2ccccc2)ccc1=O] |
| I-375 | 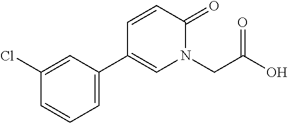 [SMILES: O=C(O)Cn1cc(-c2cccc(Cl)c2)ccc1=O] |
| I-376 | 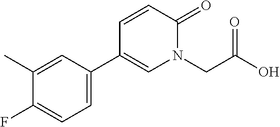 [SMILES: Cc1cc(-c2ccc(=O)n(CC(=O)O)c2)ccc1F] |
| I-377 | 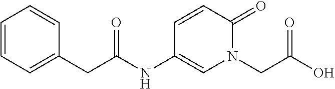 [SMILES: O=C(O)Cn1cc(NC(=O)Cc2ccccc2)ccc1=O] |
| I-378 | 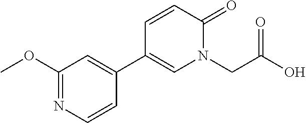 [SMILES: COc1cc(-c2ccc(=O)n(CC(=O)O)c2)ccn1] |
| I-379 | 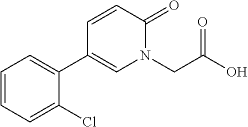 [SMILES: O=C(O)Cn1cc(-c2ccccc2Cl)ccc1=O] |
| I-380 | 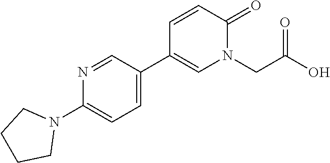 [SMILES: O=C(O)Cn1cc(-c2ccc(N3CCCC3)nc2)ccc1=O] |
| I-381 | 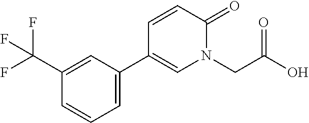 [SMILES: O=C(O)Cn1cc(-c2cccc(C(F)(F)F)c2)ccc1=O] |
| I-382 | 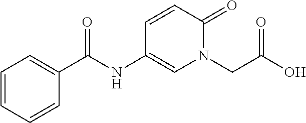 [SMILES: O=C(O)Cn1cc(NC(=O)c2ccccc2)ccc1=O] |
| I-383 | 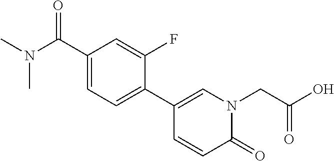 [SMILES: CN(C)C(=O)c1ccc(-c2ccc(=O)n(CC(=O)O)c2)c(F)c1] |
| I-384 | 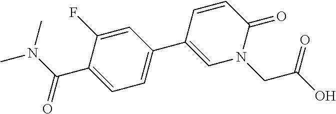 [SMILES: CN(C)C(=O)c1ccc(-c2ccc(=O)n(CC(=O)O)c2)cc1F] |
| I-385 | 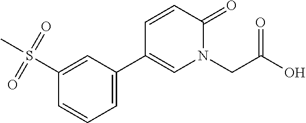 [SMILES: CS(=O)(=O)c1cccc(-c2ccc(=O)n(CC(=O)O)c2)c1] |
| I-386 | 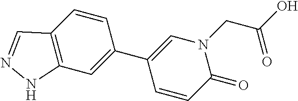 [SMILES: O=C(O)Cn1cc(-c2ccc3cn[nH]c3c2)ccc1=O] |
| I-387 | 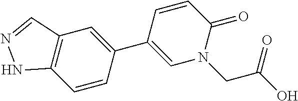 [SMILES: O=C(O)Cn1cc(-c2ccc3[nH]ncc3c2)ccc1=O] |
| I-388 | 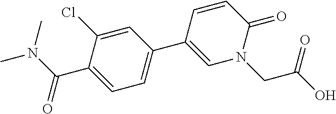 [SMILES: CN(C)C(=O)c1ccc(-c2ccc(=O)n(CC(=O)O)c2)cc1Cl] |
| I-389 | 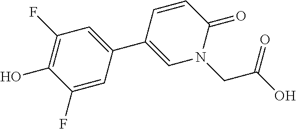 [SMILES: O=C(O)Cn1cc(-c2cc(F)c(O)c(F)c2)ccc1=O] |
| I-390 | 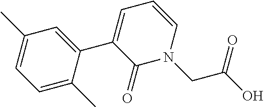 [SMILES: Cc1ccc(C)c(-c2cccn(CC(=O)O)c2=O)c1] |
| I-391 | 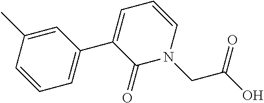 [SMILES: Cc1cccc(-c2cccn(CC(=O)O)c2=O)c1] |
| I-392 | 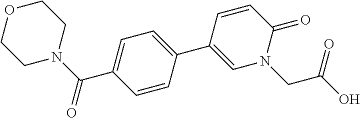 [SMILES: O=C(O)Cn1cc(-c2ccc(C(=O)N3CCOCC3)cc2)ccc1=O] |
| I-393 | 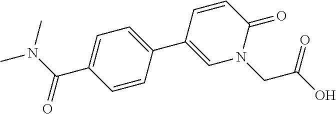 [SMILES: CN(C)C(=O)c1ccc(-c2ccc(=O)n(CC(=O)O)c2)cc1] |
| I-394 | 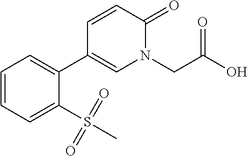 [SMILES: CS(=O)(=O)c1ccccc1-c1ccc(=O)n(CC(=O)O)c1] |
| I-395 | 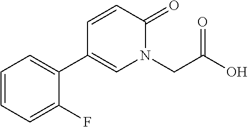 [SMILES: O=C(O)Cn1cc(-c2ccccc2F)ccc1=O] |
| I-396 | 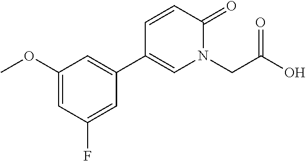 [SMILES: COc1cc(F)cc(-c2ccc(=O)n(CC(=O)O)c2)c1] |
| I-397 | 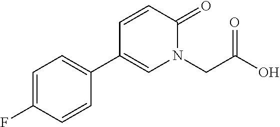 [SMILES: O=C(O)Cn1cc(-c2ccc(F)cc2)ccc1=O] |
| I-398 | 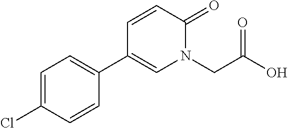 [SMILES: O=C(O)Cn1cc(-c2ccc(Cl)cc2)ccc1=O] |
| I-399 | 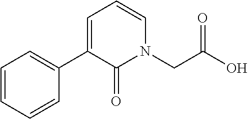 [SMILES: O=C(O)Cn1cccc(-c2ccccc2)c1=O] |
| I-400 | 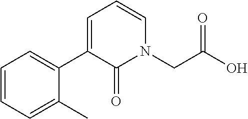 [SMILES: Cc1ccccc1-c1cccn(CC(=O)O)c1=O] |
| I-401 | 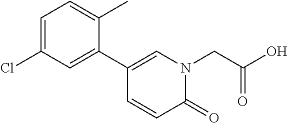 [SMILES: Cc1ccc(Cl)cc1-c1ccc(=O)n(CC(=O)O)c1] |
| I-402 | 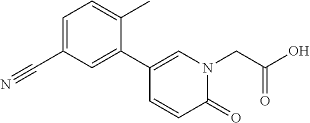 [SMILES: Cc1ccc(C#N)cc1-c1ccc(=O)n(CC(=O)O)c1] |
| I-403 | 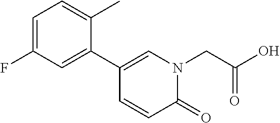 [SMILES: Cc1ccc(F)cc1-c1ccc(=O)n(CC(=O)O)c1] |
| I-404 | 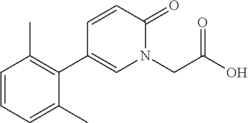 [SMILES: Cc1cccc(C)c1-c1ccc(=O)n(CC(=O)O)c1] |
| I-405 | 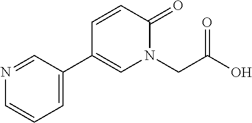 [SMILES: O=C(O)Cn1cc(-c2cccnc2)ccc1=O] |
| I-406 | 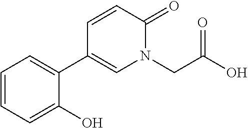 [SMILES: O=C(O)Cn1cc(-c2ccccc2O)ccc1=O] |
| I-407 | 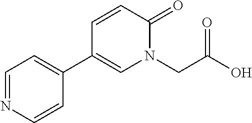 [SMILES: O=C(O)Cn1cc(-c2ccncc2)ccc1=O] |
| I-408 | 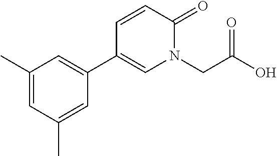 [SMILES: Cc1cc(C)cc(-c2ccc(=O)n(CC(=O)O)c2)c1] |
| I-409 | 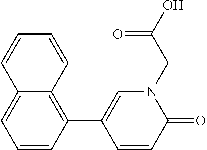 [SMILES: O=C(O)Cn1cc(-c2cccc3ccccc23)ccc1=O] |
| I-410 | 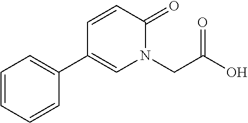 [SMILES: O=C(O)Cn1cc(-c2ccccc2)ccc1=O] |
| I-411 | 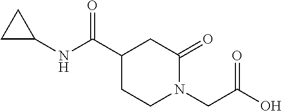 [SMILES: O=C(O)CN1CCC(C(=O)NC2CC2)CC1=O] |
| I-412 | 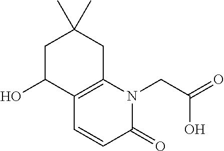 [SMILES: CC1(C)Cc2c(ccc(=O)n2CC(=O)O)C(O)C1] |
| I-413 | 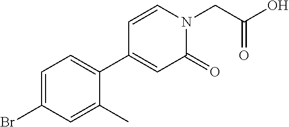 [SMILES: Cc1cc(Br)ccc1-c1ccn(CC(=O)O)c(=O)c1] |
| I-414 | 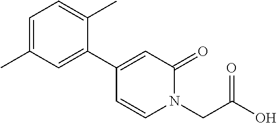 [SMILES: Cc1ccc(C)c(-c2ccn(CC(=O)O)c(=O)c2)c1] |
| I-415 | 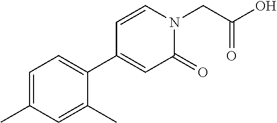 [SMILES: Cc1ccc(-c2ccn(CC(=O)O)c(=O)c2)c(C)c1] |
| I-416 | 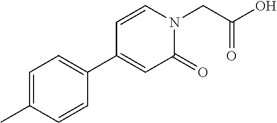 [SMILES: Cc1ccc(-c2ccn(CC(=O)O)c(=O)c2)cc1] |
| I-417 | 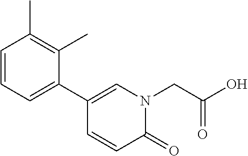 [SMILES: Cc1cccc(-c2ccc(=O)n(CC(=O)O)c2)c1C] |
| I-418 | 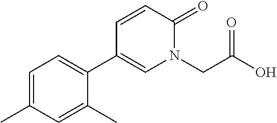 [SMILES: Cc1ccc(-c2ccc(=O)n(CC(=O)O)c2)c(C)c1] |
| I-419 | 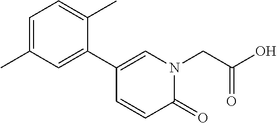 [SMILES: Cc1ccc(C)c(-c2ccc(=O)n(CC(=O)O)c2)c1] |
| I-420 | 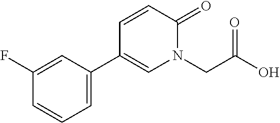 [SMILES: O=C(O)Cn1cc(-c2cccc(F)c2)ccc1=O] |
| I-421 | 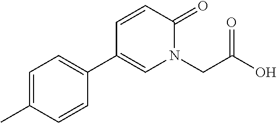 [SMILES: Cc1ccc(-c2ccc(=O)n(CC(=O)O)c2)cc1] |
| I-422 | 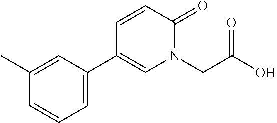 [SMILES: Cc1cccc(-c2ccc(=O)n(CC(=O)O)c2)c1] |
| I-423 | 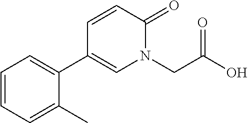 [SMILES: Cc1ccccc1-c1ccc(=O)n(CC(=O)O)c1] |
| I-424 | 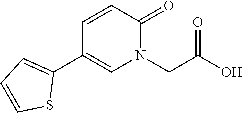 [SMILES: O=C(O)Cn1cc(-c2cccs2)ccc1=O] |
| I-425 | 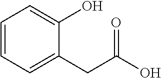 [SMILES: O=C(O)Cc1ccccc1O] |
| I-426 | 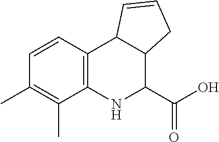 [SMILES: Cc1ccc2c(c1C)NC(C(=O)O)C1CC=CC21] |
| I-427 | 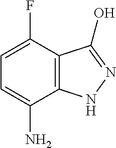 [SMILES: Nc1ccc(F)c2c(O)n[nH]c12] |
| I-428 | 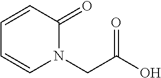 [SMILES: O=C(O)Cn1ccccc1=O] |
| I-429 | 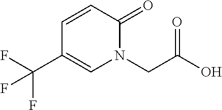 [SMILES: O=C(O)Cn1cc(C(F)(F)F)ccc1=O] |
| I-430 | 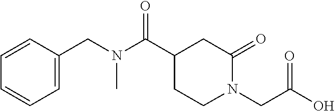 [SMILES: CN(Cc1ccccc1)C(=O)C1CCN(CC(=O)O)C(=O)C1] |
| I-431 | 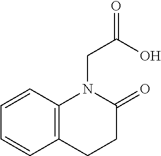 [SMILES: O=C(O)CN1C(=O)CCc2ccccc21] |
| I-432 | 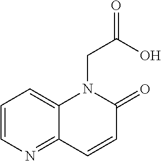 [SMILES: O=C(O)Cn1c(=O)ccc2ncccc21] |
| I-433 | 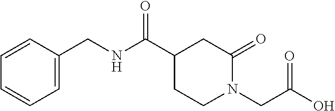 [SMILES: O=C(O)CN1CCC(C(=O)NCc2ccccc2)CC1=O] |
| I-434 | 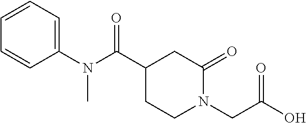 [SMILES: CN(C(=O)C1CCN(CC(=O)O)C(=O)C1)c1ccccc1] |
| I-435 | 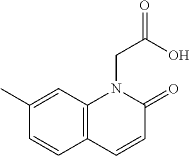 [SMILES: Cc1ccc2ccc(=O)n(CC(=O)O)c2c1] |
| I-436 | 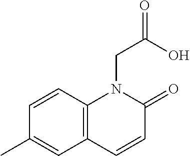 [SMILES: Cc1ccc2c(ccc(=O)n2CC(=O)O)c1] |
| I-437 | 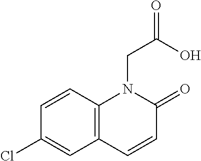 [SMILES: O=C(O)Cn1c(=O)ccc2cc(Cl)ccc21] |
| I-438 | 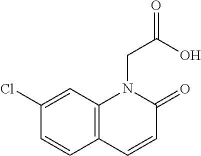 [SMILES: O=C(O)Cn1c(=O)ccc2ccc(Cl)cc21] |
| I-439 | 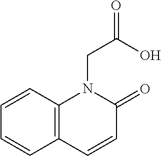 [SMILES: O=C(O)Cn1c(=O)ccc2ccccc21] |
| I-440 | 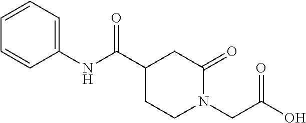 [SMILES: O=C(O)CN1CCC(C(=O)Nc2ccccc2)CC1=O] |

In some embodiments, the present invention provides a compound set forth in Table 1A, above, or a pharmaceutically acceptable salt thereof.

TABLE 1B
Exemplary Bifunctional Compounds

| I-# | Structure |
|---|---|
| I-441 | 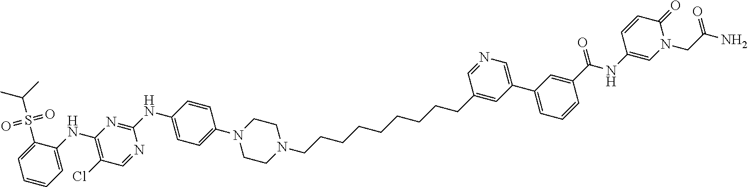 [SMILES: CC(C)S(=O)(=O)c1ccccc1Nc1nc(Nc2ccc(N3CCN(CCCCCCCCCc4cncc(-c5cccc(C(=O)Nc6ccc(=O)n(CC(N)=O)c6)c5)c4)CC3)cc2)ncc1Cl] |
| I-442 | 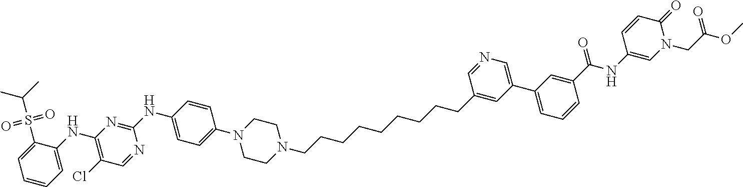 [SMILES: COC(=O)Cn1cc(NC(=O)c2cccc(-c3cncc(CCCCCCCCCN4CCN(c5ccc(Nc6ncc(Cl)c(Nc7ccccc7S(=O)(=O)C(C)C)n6)cc5)CC4)c3)c2)ccc1=O] |
| I-443 | 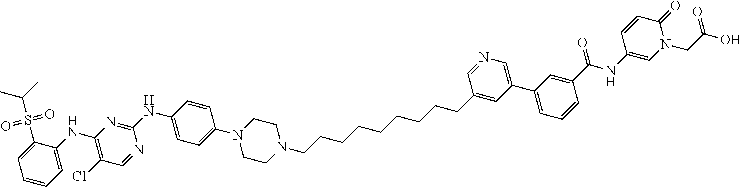 [SMILES: CC(C)S(=O)(=O)c1ccccc1Nc1nc(Nc2ccc(N3CCN(CCCCCCCCCc4cncc(-c5cccc(C(=O)Nc6ccc(=O)n(CC(=O)O)c6)c5)c4)CC3)cc2)ncc1Cl] |
| I-444 | 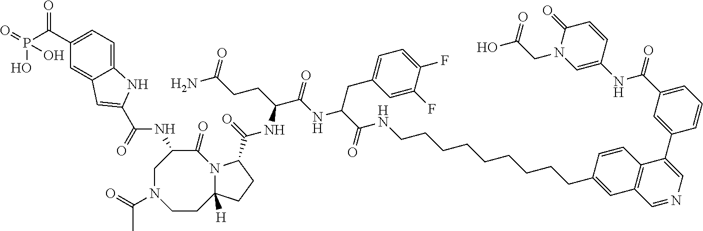 [SMILES: CC(=O)N1CC[C@H]2CC[C@@H](C(=O)N[C@@H](CCC(N)=O)C(=O)NC(Cc3ccc(F)c(F)c3)C(=O)NCCCCCCCCCc3ccc4c(-c5cccc(C(=O)Nc6ccc(=O)n(CC(=O)O)c6)c5)cncc4c3)N2C(=O)[C@@H](NC(=O)c2cc3cc(C(=O)P(=O)(O)O)ccc3[nH]2)C1] |
| I-445 | 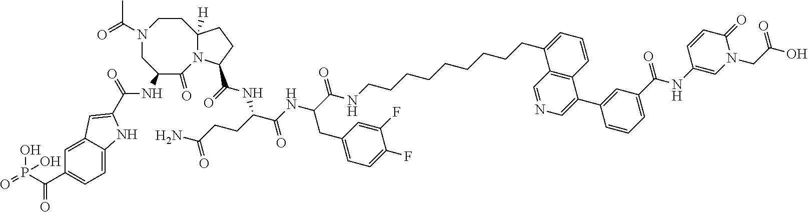 [SMILES: CC(=O)N1CC[C@H]2CC[C@@H](C(=O)N[C@@H](CCC(N)=O)C(=O)NC(Cc3ccc(F)c(F)c3)C(=O)NCCCCCCCCCc3cccc4c(-c5cccc(C(=O)Nc6ccc(=O)n(CC(=O)O)c6)c5)cncc34)N2C(=O)[C@@H](NC(=O)c2cc3cc(C(=O)P(=O)(O)O)ccc3[nH]2)C1] |
| I-446 | 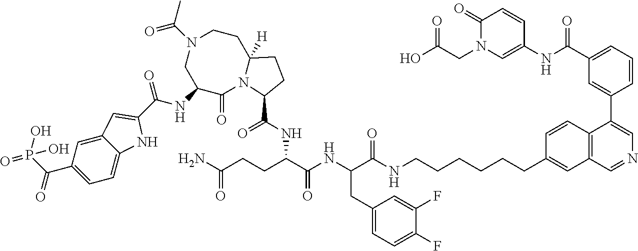 [SMILES: CC(=O)N1CC[C@H]2CC[C@@H](C(=O)N[C@@H](CCC(N)=O)C(=O)NC(Cc3ccc(F)c(F)c3)C(=O)NCCCCCCc3ccc4c(-c5cccc(C(=O)Nc6ccc(=O)n(CC(=O)O)c6)c5)cncc4c3)N2C(=O)[C@@H](NC(=O)c2cc3cc(C(=O)P(=O)(O)O)ccc3[nH]2)C1] |
| I-447 | 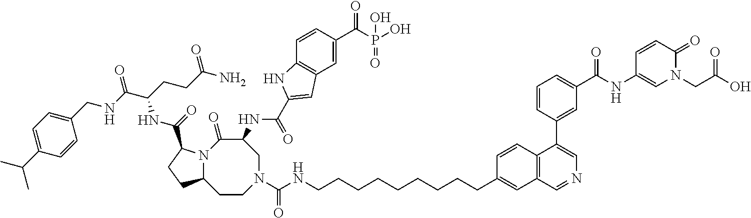 [SMILES: CC(C)c1ccc(CNC(=O)[C@H](CCC(N)=O)NC(=O)[C@@H]2CC[C@@H]3CCN(C(=O)NCCCCCCCCCc4ccc5c(-c6cccc(C(=O)Nc7ccc(=O)n(CC(=O)O)c7)c6)cncc5c4)C[C@H](NC(=O)c4cc5cc(C(=O)P(=O)(O)O)ccc5[nH]4)C(=O)N32)cc1] |
| I-448 | 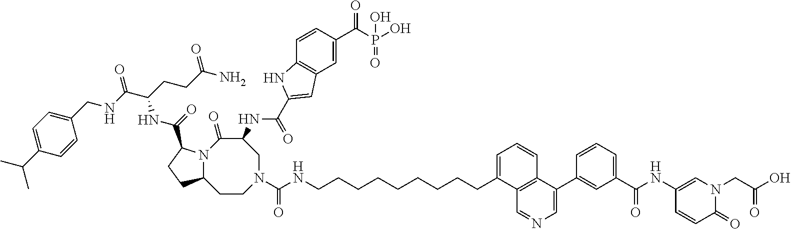 [SMILES: CC(C)c1ccc(CNC(=O)[C@H](CCC(N)=O)NC(=O)[C@@H]2CC[C@@H]3CCN(C(=O)NCCCCCCCCCc4cccc5c(-c6cccc(C(=O)Nc7ccc(=O)n(CC(=O)O)c7)c6)cncc45)C[C@H](NC(=O)c4cc5cc(C(=O)P(=O)(O)O)ccc5[nH]4)C(=O)N32)cc1] |
| I-449 | 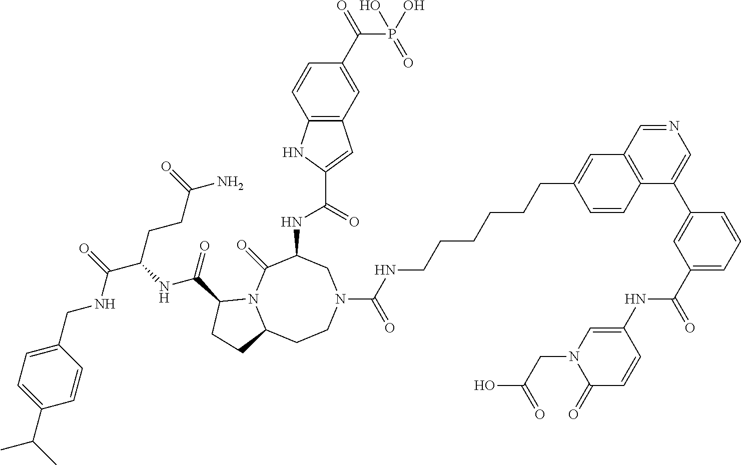 [SMILES: CC(C)c1ccc(CNC(=O)[C@H](CCC(N)=O)NC(=O)[C@@H]2CC[C@@H]3CCN(C(=O)NCCCCCCc4ccc5c(-c6cccc(C(=O)Nc7ccc(=O)n(CC(=O)O)c7)c6)cncc5c4)C[C@H](NC(=O)c4cc5cc(C(=O)P(=O)(O)O)ccc5[nH]4)C(=O)N32)cc1] |
| I-450 | 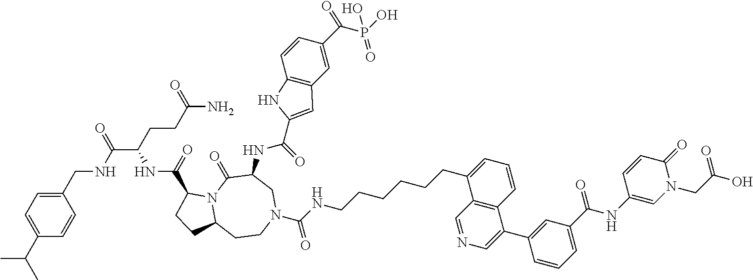 [SMILES: CC(C)c1ccc(CNC(=O)[C@H](CCC(N)=O)NC(=O)[C@@H]2CC[C@@H]3CCN(C(=O)NCCCCCCc4cccc5c(-c6cccc(C(=O)Nc7ccc(=O)n(CC(=O)O)c7)c6)cncc45)C[C@H](NC(=O)c4cc5cc(C(=O)P(=O)(O)O)ccc5[nH]4)C(=O)N32)cc1] |
| I-451 | 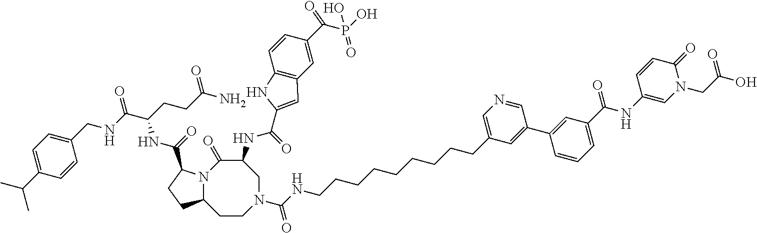 [SMILES: CC(C)c1ccc(CNC(=O)[C@H](CCC(N)=O)NC(=O)[C@@H]2CC[C@@H]3CCN(C(=O)NCCCCCCCCCc4cncc(-c5cccc(C(=O)Nc6ccc(=O)n(CC(=O)O)c6)c5)c4)C[C@H](NC(=O)c4cc5cc(C(=O)P(=O)(O)O)ccc5[nH]4)C(=O)N32)cc1] |
| I-452 | 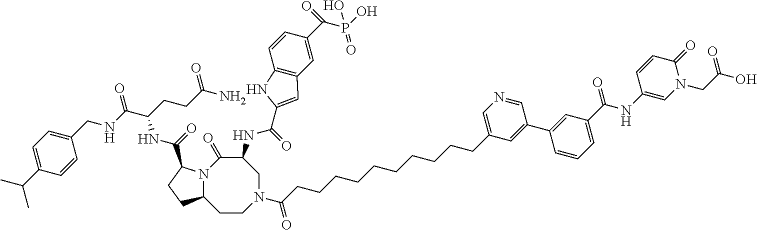 [SMILES: CC(C)c1ccc(CNC(=O)[C@H](CCC(N)=O)NC(=O)[C@@H]2CC[C@@H]3CCN(C(=O)CCCCCCCCCCc4cncc(-c5cccc(C(=O)Nc6ccc(=O)n(CC(=O)O)c6)c5)c4)C[C@H](NC(=O)c4cc5cc(C(=O)P(=O)(O)O)ccc5[nH]4)C(=O)N32)cc1] |
| I-453 | 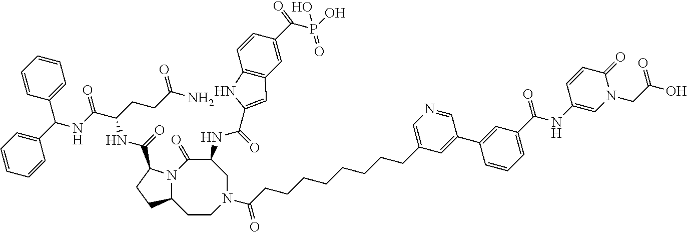 [SMILES: NC(=O)CC[C@H](NC(=O)[C@@H]1CC[C@@H]2CCN(C(=O)CCCCCCCCc3cncc(-c4cccc(C(=O)Nc5ccc(=O)n(CC(=O)O)c5)c4)c3)C[C@H](NC(=O)c3cc4cc(C(=O)P(=O)(O)O)ccc4[nH]3)C(=O)N21)C(=O)NC(c1ccccc1)c1ccccc1] |
| I-454 | 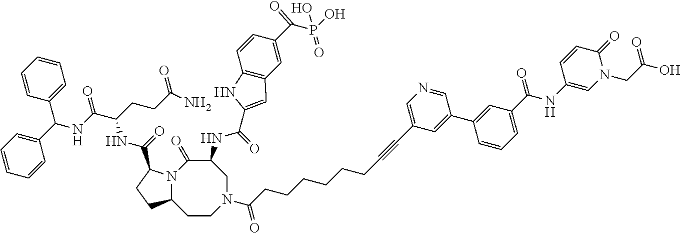 [SMILES: NC(=O)CC[C@H](NC(=O)[C@@H]1CC[C@@H]2CCN(C(=O)CCCCCCC#Cc3cncc(-c4cccc(C(=O)Nc5ccc(=O)n(CC(=O)O)c5)c4)c3)C[C@H](NC(=O)c3cc4cc(C(=O)P(=O)(O)O)ccc4[nH]3)C(=O)N21)C(=O)NC(c1ccccc1)c1ccccc1] |
| I-455 | 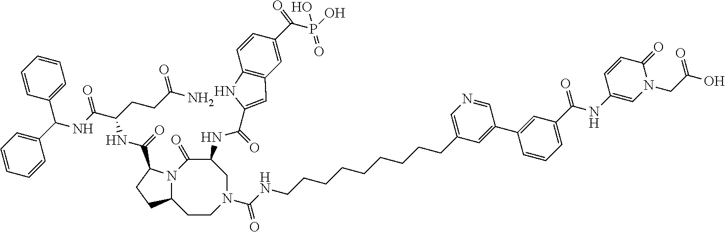 [SMILES: NC(=O)CC[C@H](NC(=O)[C@@H]1CC[C@@H]2CCN(C(=O)NCCCCCCCCCc3cncc(-c4cccc(C(=O)Nc5ccc(=O)n(CC(=O)O)c5)c4)c3)C[C@H](NC(=O)c3cc4cc(C(=O)P(=O)(O)O)ccc4[nH]3)C(=O)N21)C(=O)NC(c1ccccc1)c1ccccc1] |
| I-456 | 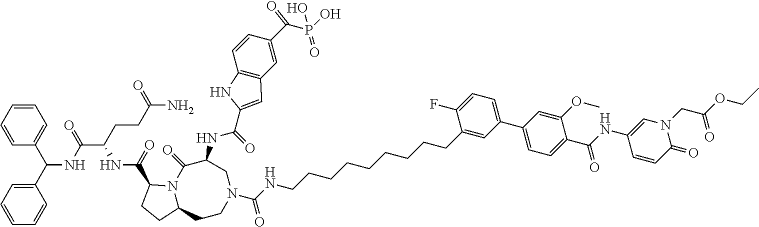 [SMILES: CCOC(=O)Cn1cc(NC(=O)c2ccc(-c3ccc(F)c(CCCCCCCCCNC(=O)N4CC[C@H]5CC[C@@H](C(=O)N[C@@H](CCC(N)=O)C(=O)NC(c6ccccc6)c6ccccc6)N5C(=O)[C@@H](NC(=O)c5cc6cc(C(=O)P(=O)(O)O)ccc6[nH]5)C4)c3)cc2OC)ccc1=O] |
| I-457 | 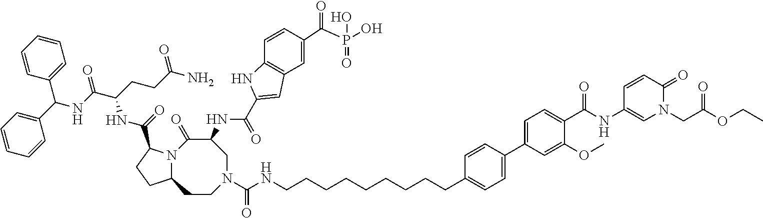 [SMILES: CCOC(=O)Cn1cc(NC(=O)c2ccc(-c3ccc(CCCCCCCCCNC(=O)N4CC[C@H]5CC[C@@H](C(=O)N[C@@H](CCC(N)=O)C(=O)NC(c6ccccc6)c6ccccc6)N5C(=O)[C@@H](NC(=O)c5cc6cc(C(=O)P(=O)(O)O)ccc6[nH]5)C4)cc3)cc2OC)ccc1=O] |
| I-458 | 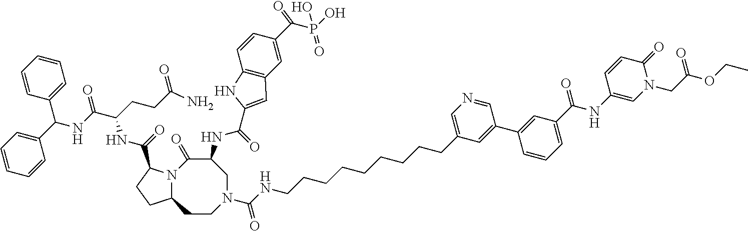 [SMILES: CCOC(=O)Cn1cc(NC(=O)c2cccc(-c3cncc(CCCCCCCCCNC(=O)N4CC[C@H]5CC[C@@H](C(=O)N[C@@H](CCC(N)=O)C(=O)NC(c6ccccc6)c6ccccc6)N5C(=O)[C@@H](NC(=O)c5cc6cc(C(=O)P(=O)(O)O)ccc6[nH]5)C4)c3)c2)ccc1=O] |
| I-459 | 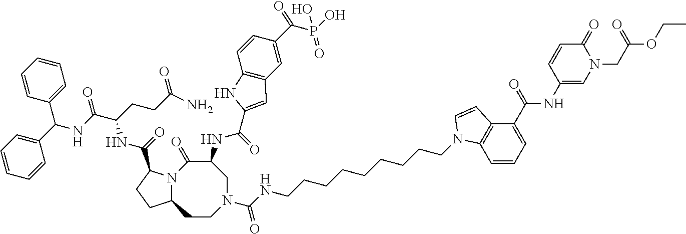 [SMILES: CCOC(=O)Cn1cc(NC(=O)c2cccc3c2ccn3CCCCCCCCCNC(=O)N2CC[C@H]3CC[C@@H](C(=O)N[C@@H](CCC(N)=O)C(=O)NC(c4ccccc4)c4ccccc4)N3C(=O)[C@@H](NC(=O)c3cc4cc(C(=O)P(=O)(O)O)ccc4[nH]3)C2)ccc1=O] |
| I-460 | 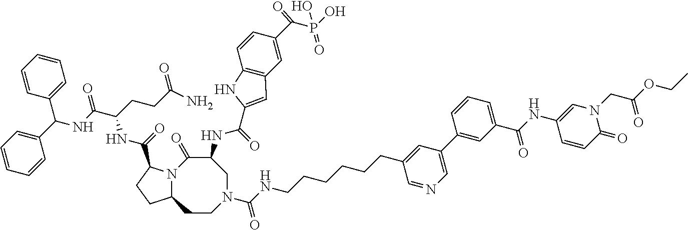 [SMILES: CCOC(=O)Cn1cc(NC(=O)c2cccc(-c3cncc(CCCCCCNC(=O)N4CC[C@H]5CC[C@@H](C(=O)N[C@@H](CCC(N)=O)C(=O)NC(c6ccccc6)c6ccccc6)N5C(=O)[C@@H](NC(=O)c5cc6cc(C(=O)P(=O)(O)O)ccc6[nH]5)C4)c3)c2)ccc1=O] |
| I-461 | 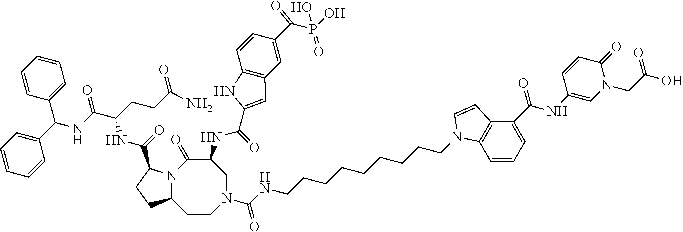 [SMILES: NC(=O)CC[C@H](NC(=O)[C@@H]1CC[C@@H]2CCN(C(=O)NCCCCCCCCCn3ccc4c(C(=O)Nc5ccc(=O)n(CC(=O)O)c5)cccc43)C[C@H](NC(=O)c3cc4cc(C(=O)P(=O)(O)O)ccc4[nH]3)C(=O)N21)C(=O)NC(c1ccccc1)c1ccccc1] |
| I-462 | 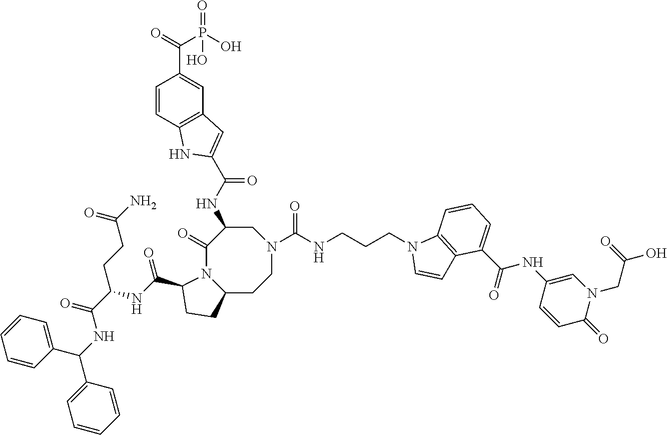 [SMILES: NC(=O)CC[C@H](NC(=O)[C@@H]1CC[C@@H]2CCN(C(=O)NCCCn3ccc4c(C(=O)Nc5ccc(=O)n(CC(=O)O)c5)cccc43)C[C@H](NC(=O)c3cc4cc(C(=O)P(=O)(O)O)ccc4[nH]3)C(=O)N21)C(=O)NC(c1ccccc1)c1ccccc1] |
| I-463 | 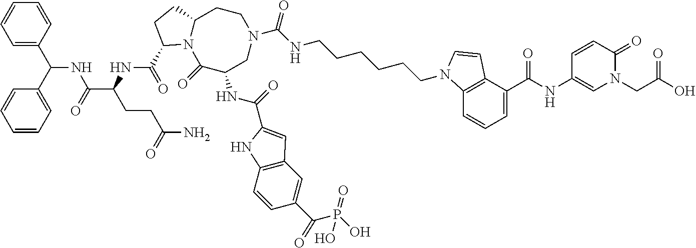 [SMILES: NC(=O)CC[C@H](NC(=O)[C@@H]1CC[C@@H]2CCN(C(=O)NCCCCCCn3ccc4c(C(=O)Nc5ccc(=O)n(CC(=O)O)c5)cccc43)C[C@H](NC(=O)c3cc4cc(C(=O)P(=O)(O)O)ccc4[nH]3)C(=O)N21)C(=O)NC(c1ccccc1)c1ccccc1] |
| I-464 | 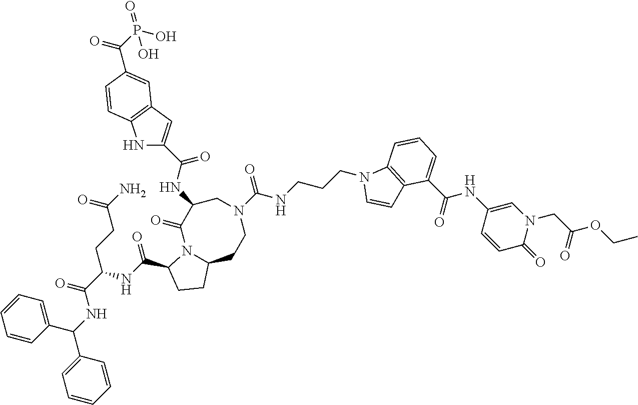 [SMILES: CCOC(=O)Cn1cc(NC(=O)c2cccc3c2ccn3CCCNC(=O)N2CC[C@H]3CC[C@@H](C(=O)N[C@@H](CCC(N)=O)C(=O)NC(c4ccccc4)c4ccccc4)N3C(=O)[C@@H](NC(=O)c3cc4cc(C(=O)P(=O)(O)O)ccc4[nH]3)C2)ccc1=O] |
| I-465 | 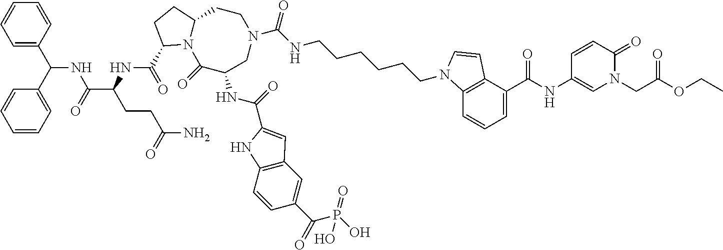 [SMILES: CCOC(=O)Cn1cc(NC(=O)c2cccc3c2ccn3CCCCCCNC(=O)N2CC[C@H]3CC[C@@H](C(=O)N[C@@H](CCC(N)=O)C(=O)NC(c4ccccc4)c4ccccc4)N3C(=O)[C@@H](NC(=O)c3cc4cc(C(=O)P(=O)(O)O)ccc4[nH]3)C2)ccc1=O] |
| I-466 | 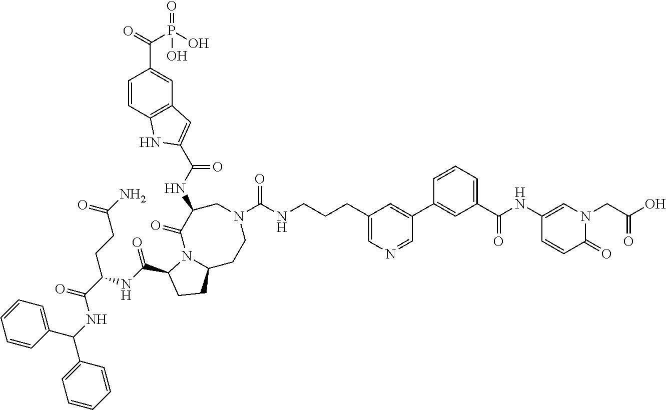 [SMILES: NC(=O)CC[C@H](NC(=O)[C@@H]1CC[C@@H]2CCN(C(=O)NCCCc3cncc(-c4cccc(C(=O)Nc5ccc(=O)n(CC(=O)O)c5)c4)c3)C[C@H](NC(=O)c3cc4cc(C(=O)P(=O)(O)O)ccc4[nH]3)C(=O)N21)C(=O)NC(c1ccccc1)c1ccccc1] |
| I-467 | 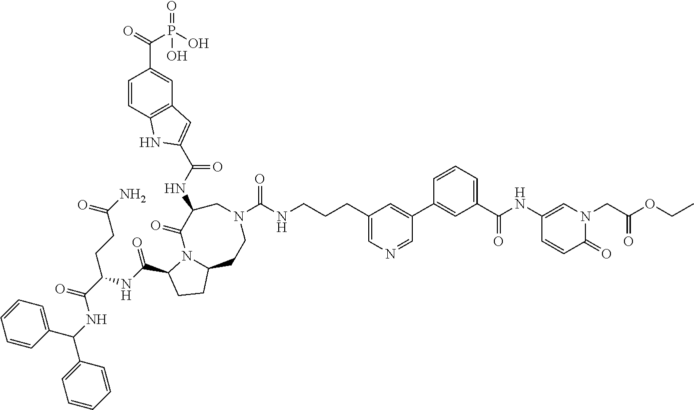 [SMILES: CCOC(=O)Cn1cc(NC(=O)c2cccc(-c3cncc(CCCNC(=O)N4CC[C@H]5CC[C@@H](C(=O)N[C@@H](CCC(N)=O)C(=O)NC(c6ccccc6)c6ccccc6)N5C(=O)[C@@H](NC(=O)c5cc6cc(C(=O)P(=O)(O)O)ccc6[nH]5)C4)c3)c2)ccc1=O] |
| I-468 | 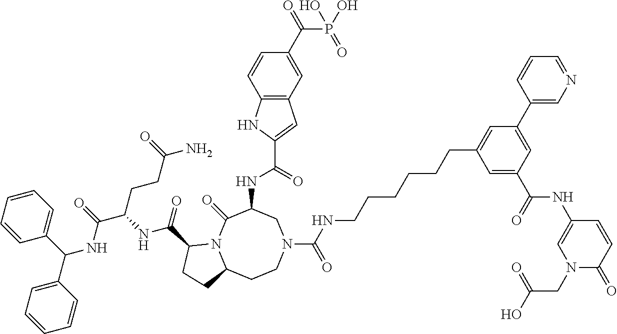 [SMILES: NC(=O)CC[C@H](NC(=O)[C@@H]1CC[C@@H]2CCN(C(=O)NCCCCCCc3cc(C(=O)Nc4ccc(=O)n(CC(=O)O)c4)cc(-c4cccnc4)c3)C[C@H](NC(=O)c3cc4cc(C(=O)P(=O)(O)O)ccc4[nH]3)C(=O)N21)C(=O)NC(c1ccccc1)c1ccccc1] |
| I-469 | 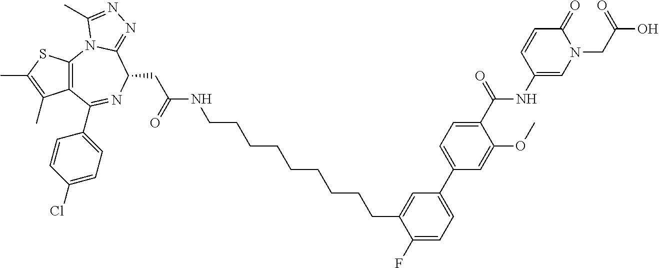 [SMILES: COc1cc(-c2ccc(F)c(CCCCCCCCCNC(=O)C[C@@H]3N=C(c4ccc(Cl)cc4)c4c(sc(C)c4C)-n4c(C)nnc43)c2)ccc1C(=O)Nc1ccc(=O)n(CC(=O)O)c1] |
| I-470 | 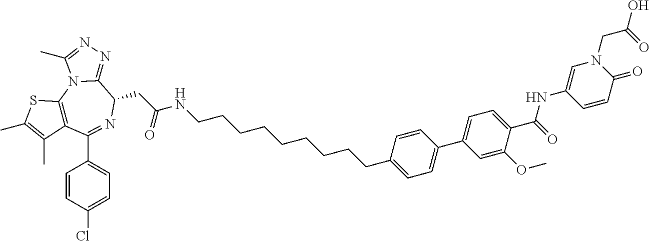 [SMILES: COc1cc(-c2ccc(CCCCCCCCCNC(=O)C[C@@H]3N=C(c4ccc(Cl)cc4)c4c(sc(C)c4C)-n4c(C)nnc43)cc2)ccc1C(=O)Nc1ccc(=O)n(CC(=O)O)c1] |
| I-471 | 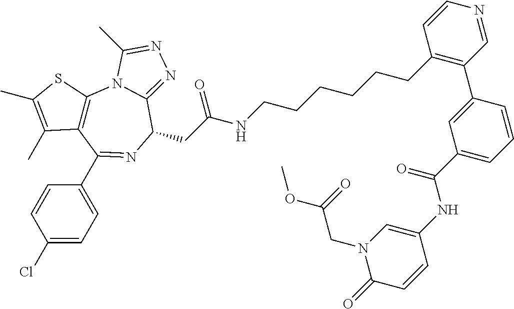 [SMILES: COC(=O)Cn1cc(NC(=O)c2cccc(-c3cnccc3CCCCCCNC(=O)C[C@@H]3N=C(c4ccc(Cl)cc4)c4c(sc(C)c4C)-n4c(C)nnc43)c2)ccc1=O] |
| I-472 | 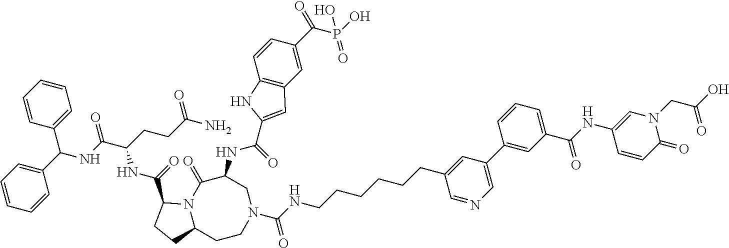 [SMILES: NC(=O)CC[C@H](NC(=O)[C@@H]1CC[C@@H]2CCN(C(=O)NCCCCCCc3cncc(-c4cccc(C(=O)Nc5ccc(=O)n(CC(=O)O)c5)c4)c3)C[C@H](NC(=O)c3cc4cc(C(=O)P(=O)(O)O)ccc4[nH]3)C(=O)N21)C(=O)NC(c1ccccc1)c1ccccc1] |
| I-473 | 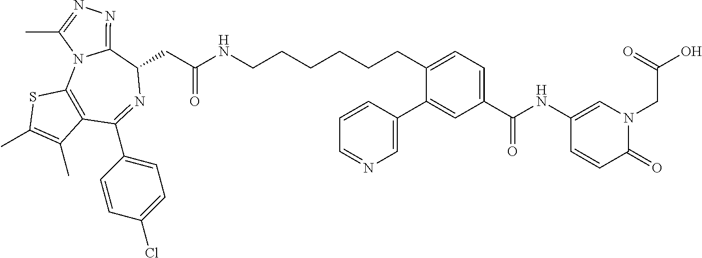 [SMILES: Cc1sc2c(c1C)C(c1ccc(Cl)cc1)=N[C@@H](CC(=O)NCCCCCCc1ccc(C(=O)Nc3ccc(=O)n(CC(=O)O)c3)cc1-c1cccnc1)c1nnc(C)n1-2] |
| I-474 | 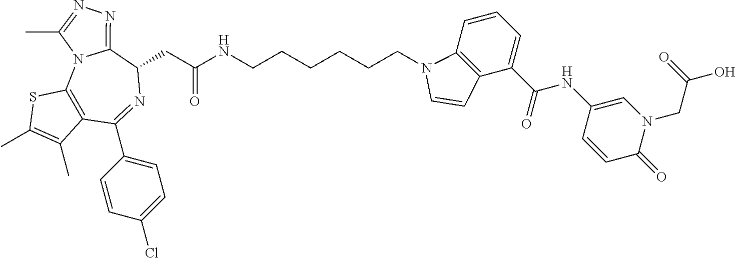 [SMILES: Cc1sc2c(c1C)C(c1ccc(Cl)cc1)=N[C@@H](CC(=O)NCCCCCCn1ccc3c(C(=O)Nc4ccc(=O)n(CC(=O)O)c4)cccc31)c1nnc(C)n1-2] |
| I-475 | 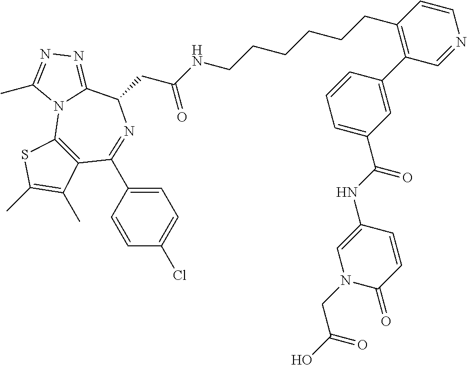 [SMILES: Cc1sc2c(c1C)C(c1ccc(Cl)cc1)=N[C@@H](CC(=O)NCCCCCCc1ccncc1-c1cccc(C(=O)Nc3ccc(=O)n(CC(=O)O)c3)c1)c1nnc(C)n1-2] |
| I-476 | 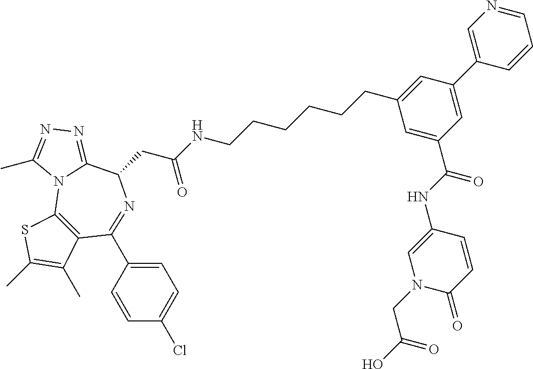 [SMILES: Cc1sc2c(c1C)C(c1ccc(Cl)cc1)=N[C@@H](CC(=O)NCCCCCCc1cc(C(=O)Nc3ccc(=O)n(CC(=O)O)c3)cc(-c3cccnc3)c1)c1nnc(C)n1-2] |
| I-477 | 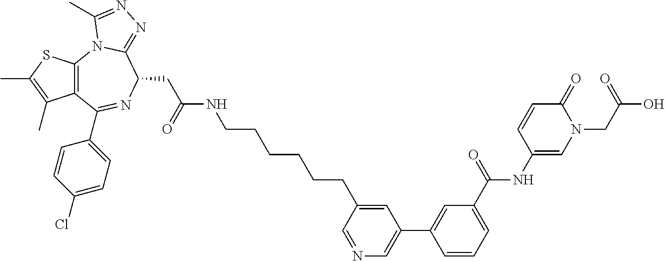 [SMILES: Cc1sc2c(c1C)C(c1ccc(Cl)cc1)=N[C@@H](CC(=O)NCCCCCCc1cncc(-c3cccc(C(=O)Nc4ccc(=O)n(CC(=O)O)c4)c3)c1)c1nnc(C)n1-2] |
| I-478 | 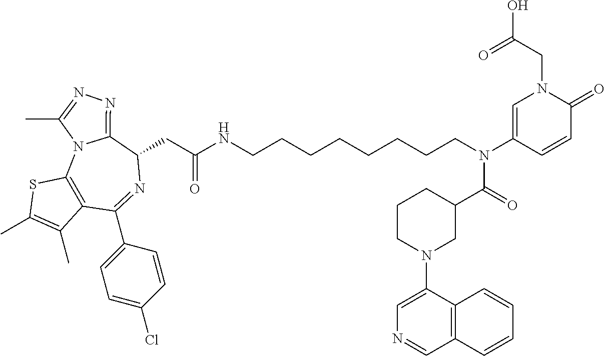 [SMILES: Cc1sc2c(c1C)C(c1ccc(Cl)cc1)=N[C@@H](CC(=O)NCCCCCCCCN(C(=O)C1CCCN(c3cncc4ccccc34)C1)c1ccc(=O)n(CC(=O)O)c1)c1nnc(C)n1-2] |
| I-479 | 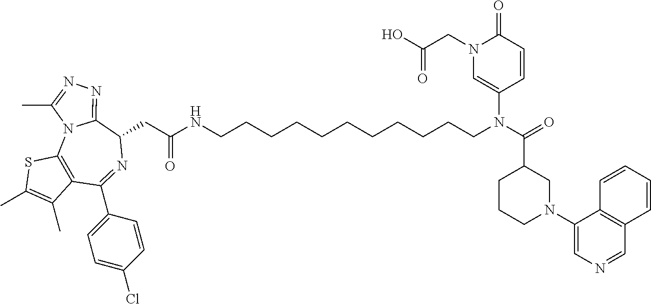 [SMILES: Cc1sc2c(c1C)C(c1ccc(Cl)cc1)=N[C@@H](CC(=O)NCCCCCCCCCCCN(C(=O)C1CCCN(c3cncc4ccccc34)C1)c1ccc(=O)n(CC(=O)O)c1)c1nnc(C)n1-2] |
| I-480 | 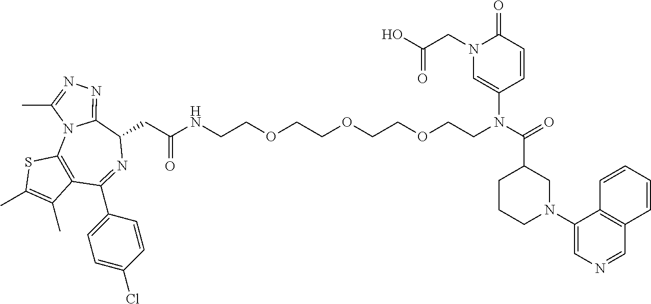 [SMILES: Cc1sc2c(c1C)C(c1ccc(Cl)cc1)=N[C@@H](CC(=O)NCCOCCOCCOCCN(C(=O)C1CCCN(c3cncc4ccccc34)C1)c1ccc(=O)n(CC(=O)O)c1)c1nnc(C)n1-2] |
| I-481 | 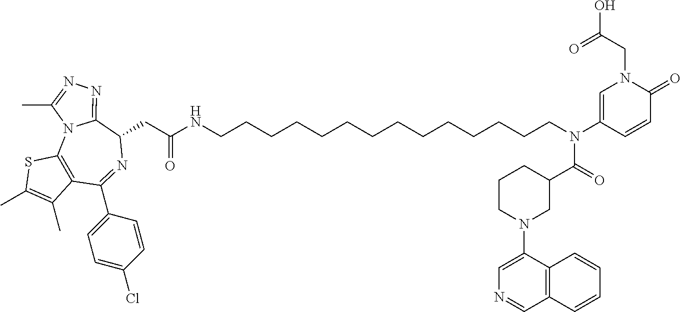 [SMILES: Cc1sc2c(c1C)C(c1ccc(Cl)cc1)=N[C@@H](CC(=O)NCCCCCCCCCCCCCCN(C(=O)C1CCCN(c3cncc4ccccc34)C1)c1ccc(=O)n(CC(=O)O)c1)c1nnc(C)n1-2] |
| I-482 | 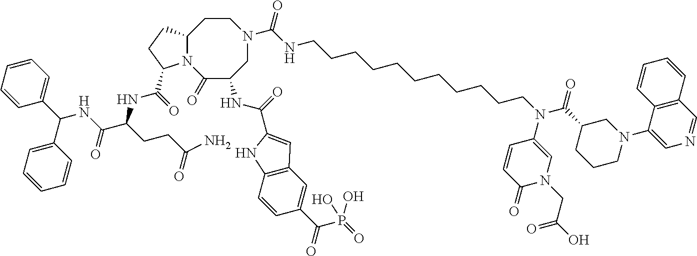 [SMILES: NC(=O)CC[C@H](NC(=O)[C@@H]1CC[C@@H]2CCN(C(=O)NCCCCCCCCCCCN(C(=O)[C@H]3CCCN(c4cncc5ccccc45)C3)c3ccc(=O)n(CC(=O)O)c3)C[C@H](NC(=O)c3cc4cc(C(=O)P(=O)(O)O)ccc4[nH]3)C(=O)N21)C(=O)NC(c1ccccc1)c1ccccc1] |
| I-483 | 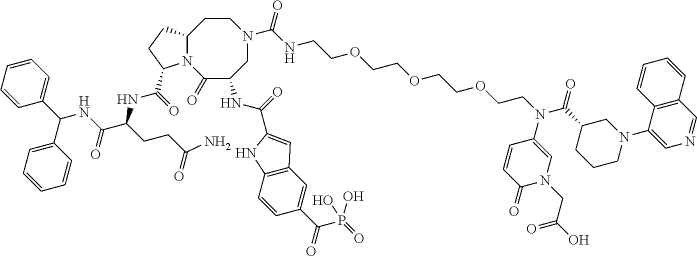 [SMILES: NC(=O)CC[C@H](NC(=O)[C@@H]1CC[C@@H]2CCN(C(=O)NCCOCCOCCOCCN(C(=O)[C@H]3CCCN(c4cncc5ccccc45)C3)c3ccc(=O)n(CC(=O)O)c3)C[C@H](NC(=O)c3cc4cc(C(=O)P(=O)(O)O)ccc4[nH]3)C(=O)N21)C(=O)NC(c1ccccc1)c1ccccc1] |
| I-484 | 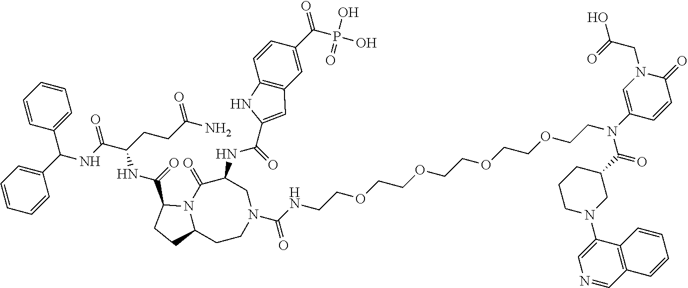 [SMILES: NC(=O)CC[C@H](NC(=O)[C@@H]1CC[C@@H]2CCN(C(=O)NCCOCCOCCOCCOCCN(C(=O)[C@H]3CCCN(c4cncc5ccccc45)C3)c3ccc(=O)n(CC(=O)O)c3)C[C@H](NC(=O)c3cc4cc(C(=O)P(=O)(O)O)ccc4[nH]3)C(=O)N21)C(=O)NC(c1ccccc1)c1ccccc1] |
| I-485 | 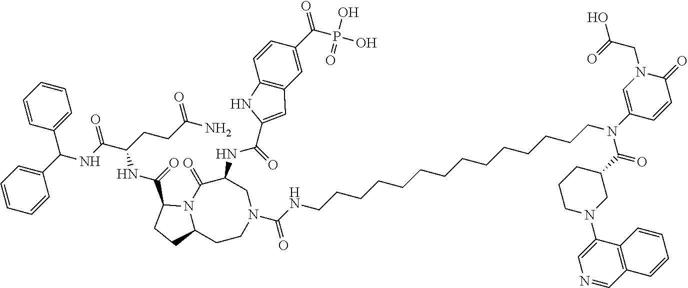 [SMILES: NC(=O)CC[C@H](NC(=O)[C@@H]1CC[C@@H]2CCN(C(=O)NCCCCCCCCCCCCCCN(C(=O)[C@H]3CCCN(c4cncc5ccccc45)C3)c3ccc(=O)n(CC(=O)O)c3)C[C@H](NC(=O)c3cc4cc(C(=O)P(=O)(O)O)ccc4[nH]3)C(=O)N21)C(=O)NC(c1ccccc1)c1ccccc1] |

In some embodiments, the present invention provides a compound set forth in Table 1B, above, or a pharmaceutically acceptable salt thereof.

In some embodiments, TBM is one of the compounds in Table 2, below, wherein

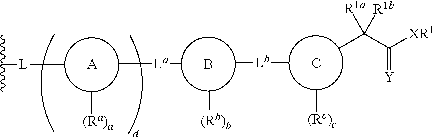

is attached to a modifiable carbon, oxygen, nitrogen or sulfur atom.

TABLE 2
Exemplary Drugs with Disease Indications and Gene Identifier for the Target Protein

| Drug Name | Indication(s) | Gene |
|---|---|---|
| 3196 | anticholesterolaemic agent | THRB |
| Posiphen | for treatment of Alzheimer's disease | APP |
| Posiphen | for treatment of Alzheimer's disease | BACE1 |
| MBO7133 (cytarabine prodrug) | antineoplastic agent | POLB |
| 4SC-202 | antineoplastic agent | HDAC1 |
| 4SC-202 | antineoplastic agent | HDAC2 |
| 4SC-202 | antineoplastic agent | HDAC3 |
| 4SC-202 | antineoplastic agent | HDAC8 |
| 4SC-202 | antineoplastic agent | FLT3 |
| 4SC-202 | antineoplastic agent | VEGFA |
| 4SC-205 | antineoplastic agent | KIF11 |
| 768974 | antiosteoporotic agent | PTH1R |
| 7a-methyl-19-nortestosterone, | hormone replacement, male contraceptive | AR |
| MENT | | |
| A-007 | antineoplastic agent | ESR1 |
| A-007 | antineoplastic agent | ESR2 |
| oxybutynin | for treatment of incontinence | CHRM1 |
| oxybutynin | for treatment of incontinence | CHRM2 |
| oxybutynin | for treatment of incontinence | CHRM3 |
| Testosterone | hormone replacement | AR |
| ABC294640 | antineoplastic agent | SPHK1 |
| ABC294640 | antineoplastic agent | SPHK2 |
| Aripiprazole | antipsychotic agent | DRD2 |
| Aripiprazole | antipsychotic agent | HTR1A |
| Aripiprazole | antipsychotic agent | HTR2A |
| paclitaxel | antineoplastic agent | BCL2 |
| paclitaxel | antineoplastic agent | TUBB1 |
| navitoclax, ABT-263 | antineoplastic agent | BCL2 |
| navitoclax, ABT-263 | antineoplastic agent | BCL2L1 |
| navitoclax, ABT-263 | antineoplastic agent | BCL2L2 |
| fenofibrate | antidyslipidaemic agent | PPARA |
| Linifanib | antineoplastic agent | CSF1R |
| Linifanib | antineoplastic agent | FLT1 |
| Linifanib | antineoplastic agent | FLT3 |
| Linifanib | antineoplastic agent | FLT4 |
| Linifanib | antineoplastic agent | KDR |
| Linifanib | antineoplastic agent | KIT |
| Linifanib | antineoplastic agent | PDGFRB |
| Linifanib | antineoplastic agent | RET |
| Linifanib | antineoplastic agent | TIE2 |
| AC-201 | antidiabetic | IL1B |
| AC-201 | antidiabetic | IL1RN |
| quizartinib | antineoplastic agent | FLT3 |
| AC430 | antiinflammatory agent, antineoplastic agent | JAK2 |
| AC480 | antineoplastic agent | EGFR |
| AC480 | antineoplastic agent | ERBB2 |
| AC480 | antineoplastic agent | ERBB3 |
| AC480 | antineoplastic agent | ERBB4 |
| acamprosate | for treatment of alcohol-dependance | GRIN3A |
| acamprosate | antineoplastic agent | GRM5 |
| toremifene | antineoplastic agent, SERM | ESR1 |
| acarbose | antidiabetic | AMY2A |
| acarbose | antidiabetic | GAA |
| acarbose | antidiabetic | MGAM |
| acarbose | antidiabetic | SI |
| organic nitrate + 1-arginine | vasodilator | NOS3 |
| Acccretropin | for treatment of turner's syndrome | GHR |
| rabeprazole | Proton pump inhibitor | ATP4A |
| aclidinium | bronchodilator | CHRM1 |
| aclidinium | bronchodilator | CHRM2 |
| aclidinium | bronchodilator | CHRM3 |
| aclidinium | bronchodilator | CHRM4 |
| aclidinium | bronchodilator | CHRM5 |
| acotiamide | for treatment of functional dyspepsia | ACHE |
| ACP-001 | hormone replacement | GHR |
| ACP-104 | antipsychotic agent | CHRM1 |
| ACP-104 | antipsychotic agent | DRD2 |
| ACP-104 | antipsychotic agent | DRD3 |
| ACP-104 | antipsychotic agent | HTR2A |
| ACTB1003 | antineoplastic agent | FGFR1 |
| ACTB1003 | antineoplastic agent | FGFR2 |
| ACTB1003 | antineoplastic agent | FGFR3 |
| ACTB1003 | antineoplastic agent | FGFR4 |
| ACTB1003 | antineoplastic agent | RPS6KB1 |
| ACY-1215 | antineoplastic agent | HDAC6 |
| AD 337 | analgesic, for treatment of fibromyalgia | SLC6A2 |
| AD 337 | analgesic, for treatment of fibromyalgia | SLC6A4 |
| fentanyl | analgesic | OPRD1 |
| fentanyl | analgesic | OPRM1 |
| theophylline | bronchodilator | ADORA1 |
| theophylline | bronchodilator | ADORA2A |
| theophylline | bronchodilator | ADORA2B |
| theophylline | bronchodilator | PDE3A |
| theophylline | bronchodilator | PDE4A |
| theophylline | bronchodilator | PDE4B |
| theophylline | bronchodilator | PDE5A |
| ADL5747 | analgesic | OPRDI |
| ADL5859 | analgesic | OPRD1 |
| ADL5945 | motilitant | OPRM1 |
| ADL7445 | motilitant | OPRM1 |
| capsaicin | analgesic | TRPV1 |
| fluticasone propionate | bronchodilator | NR3C1 |
| salmeterol | bronchodilator | ADRB2 |
| ADX10059 | antimigraine agent, for treatment of | GRM5 |
| | gastroesophageal reflux disease | |
| ADX415 | antihypertensive agent | ADRA2A |
| ADX-71149 | antipsychotic agent, antidepressant, anxiolytic | GRM2 |
| fentanyl | analgesic | OPRD1 |
| fentanyl | analgesic | OPRM1 |
| AES-103 | for treatment of sickle-cell disease | HBB |
| doxorubicin | antineoplastic agent | TOP2A |
| AEZS-112, ZEN-012 | antineoplastic agent | TOP2A |
| AEZS-112, ZEN-012 | antineoplastic agent | TUBB |
| AEZS-112, ZEN-012 | antineoplastic agent | TUBB1 |
| Afamelanotide | dermatological agent | MC1R |
| afatinib | antineoplastic agent | EGFR |
| afatinib | antineoplastic agent | ERBB2 |
| ethinyl estradiol | contraceptive | ESR1 |
| levonorgestrel | contraceptive | ESR1 |
| levonorgestrel | contraceptive | PGR |
| levonorgestrel | contraceptive | SRD5A1 |
| mecamylamine | motilitant | CHRNA2 |
| AGI-1067, succinobucol | antiatherosclerosis agent | VCAM1 |
| AGIX-4207 | antiinflammatory agent, DMARD | unknown |
| AGN-214868 | analgesic, neuralgia | ADRA1A |
| AGN-214868 | analgesic, neuralgia | ADRA1B |
| AGN-214868 | analgesic, neuralgia | ADRA1D |
| AGN-214868 | analgesic, neuralgia | ADRA2A |
| AGN-214868 | analgesic, neuralgia | ADRA2B |
| AGN-214868 | analgesic, neuralgia | ADRA2C |
| agomelatine | antidepressant | MTNR1B |
| agomelatine | antidepressant | HTR2B |
| agomelatine | antidepressant | HTR2C |
| agomelatine | antidepressant | MTNR1A |
| hydroxychloroquine | antirheumatic agent | TLR7 |
| hydroxychloroquine | antirheumatic agent | TLR9 |
| paclitaxel | antineoplastic agent | BCL2 |
| paclitaxel | antineoplastic agent | TUBB1 |
| AIKO-150 | opioid antagonist | OPRM1 |
| AIR645 | antiasthmatic agent | IL4RA |
| AKB-6548 | for treatment of anaemia | EGLN1 |
| AKB-6548 | for treatment of anaemia | EGLN2 |
| AKL-0707 | hormone replacement | GHRH |
| ALB109564(a) | antincoplastic agent | TUBB |
| ALB-127158(a) | antiobesity agent | MCHR1 |
| salbutamol | bronchodilator | ADRB2 |
| aleglitazar | cardiovascular agent | PPARA |
| aleglitazar | cardiovascular agent | PPARG |
| alfuzosin | for treatment of benign prostatic hyperplasia | ADRA1A |
| alfuzosin | for treatment of benign prostatic hyperplasia | ADRA1B |
| alfuzosin | for treatment of benign prostatic hyperplasia | ADRA1D |
| lidocaine | anesthetic | SCN10A |
| lidocaine | anesthetic | SCN5A |
| lidocaine | anesthetic | SCN9A |
| pemetrexed | antineoplastic agent | DHFR |
| pemetrexed | antineoplastic agent | GART |
| pemetrexed | antineoplastic agent | TYMS |
| aliskiren | antihypertensive agent | REN |
| aliskiren | antihypertensive agent | REN |
| amlodipine | antihypertensive agent | CACNA1C |
| amlodipine | antihypertensive agent | CACNA1D |
| amlodipine | antihypertensive agent | CACNA1S |
| amlodipine | antihypertensive agent | CACNA2D1 |
| amlodipine | antihypertensive agent | CACNB2 |
| Alitretionine | antineoplastic agent | RARA |
| Alitretionine | antineoplastic agent | RARB |
| Alitretionine | antineoplastic agent | RARG |
| Alitretionine | antineoplastic agent | RXRA |
| Alitretionine | antineoplastic agent | RXRB |
| Alitretionine | antineoplastic agent | RXRG |
| Alitretionine | antineoplastic agent | RARA |
| Alitretionine | antineoplastic agent | RARB |
| Alitretionine | antineoplastic agent | RARG |
| Alitretionine | antineoplastic agent | RXRA |
| Alitretionine | antineoplastic agent | RXRB |
| Alitretionine | antineoplastic agent | RXRG |
| ALKS 33 | for treatment of alcohol | OPRD1 |
| | dependance, antidepressant | |
| ALKS 33 | for treatment of alcohol | OPRK1 |
| | dependance, antidepressant | |
| ALKS 33 | for treatment of alcohol | OPRM1 |
| | dependance, antidepressant | |
| baclofen | for treatment of alcohol dependance | GABBR1 |
| baclofen | for treatment of alcohol dependance | GABBR2 |
| ALKS 33 | for treatment of alcohol | OPRD1 |
| | dependance, antidepressant | |
| ALKS 33 | for treatment of alcohol | OPRK1 |
| | dependance, antidepressant | |
| ALKS 33 | for treatment of alcohol | OPRM1 |
| | dependance, antidepressant | |
| ALKS 37 | motilitant | OPRD1 |
| ALKS 37 | motilitant | OPRK1 |
| ALKS 37 | motilitant | OPRM1 |
| ALKS 33 | for treatment of alcohol | OPRD1 |
| | dependance, antidepressant | |
| ALKS 33 | for treatment of alcohol | OPRK1 |
| | dependance, antidepressant | |
| ALKS 33 | for treatment of alcohol | OPRM1 |
| | dependance, antidepressant | |
| buprenorphine | antidepressant, analgesic, for treatment of opioid | OPRD1 |
| | addiction | |
| buprenorphine | antidepressant, analgesic, for treatment of opioid | OPRK1 |
| | addiction | |
| almorexant | sleep disorder treatment | HCRTR1 |
| almorexant | sleep disorder treatment | HCRTR2 |
| almotriptan | antimigraine agent | HTR1B |
| almotriptan | antimigraine agent | HTR1D |
| morphine | analgesic | OPRD1 |
| morphine | analgesic | OPRD1 |
| morphine | analgesic | OPRK1 |
| morphine | analgesic | OPRK1 |
| morphine | analgesic | OPRM1 |
| morphine | analgesic | OPRM1 |
| naltrexone | analgesic | OPRD1 |
| naltrexone | analgesic | OPRD1 |
| naltrexone | analgesic | OPRK1 |
| naltrexone | analgesic | OPRK1 |
| naltrexone | analgesic | OPRM1 |
| naltrexone | analgesic | OPRM1 |
| naltrexone | analgesic | SIGMAR1 |
| alogliptin | antidiabetic | DPP4 |
| alosetron | for treatment of irritable bowel syndrome | HTR3A |
| alprazolam | anxiolytic, sedative, hypnotic | GABRA1 |
| alprazolam | anxiolytic, sedative, hypnotic | GABRA2 |
| alprazolam | anxiolytic, sedative, hypnotic | GABRA3 |
| alprazolam | anxiolytic, sedative, hypnotic | GABRA4 |
| alprazolam | anxiolytic, sedative, hypnotic | GABRA5 |
| alprazolam | anxiolytic, sedative, hypnotic | GABRA6 |
| alprazolam | anxiolytic, sedative, hypnotic | GABRB1 |
| alprazolam | anxiolytic, sedative, hypnotic | GABRB2 |
| alprazolam | anxiolytic, sedative, hypnotic | GABRB3 |
| alprazolam | anxiolytic, sedative, hypnotic | GABRD |
| alprazolam | anxiolytic, sedative, hypnotic | GABRE |
| alprazolam | anxiolytic, sedative, hypnotic | GABRG1 |
| alprazolam | anxiolytic, sedative, hypnotic | GABRG2 |
| alprazolam | anxiolytic, sedative, hypnotic | GABRG3 |
| alprazolam | anxiolytic, sedative, hypnotic | GABRP |
| alprazolam | anxiolytic, sedative, hypnotic | GABRQ |
| alprazolam | anxiolytic, sedative, hypnotic | GABRR2 |
| alprazolam | anxiolytic, sedative, hypnotic | GABRR3 |
| alprostadil | for treatment of erectile dysfunction, for | PTGER1 |
| | treatment of sexual dysfunction in women | |
| alprostadil | for treatment of erectile dysfunction, for | PTGER2 |
| | treatment of sexual dysfunction in women | |
| alprostadil | for treatment of erectile dysfunction, for | PTGER1 |
| | treatment of sexual dysfunction in women | |
| alprostadil | for treatment of erectile dysfunction, for | PTGER2 |
| | treatment of sexual dysfunction in women | |
| alprostadil | for treatment of erectile dysfunction, for | PTGER1 |
| | treatment of sexual dysfunction in women | |
| alprostadil | for treatment of erectile dysfunction, for | PTGER2 |
| | treatment of sexual dysfunction in women | |
| altropane | diagnostic agent for parkinson's disease and | SLC6A3 |
| | ADHD | |
| Alvespimycin | antineoplastic agent | HSP90AA1 |
| Alvespimycin | antineoplastic agent | HSP90AB1 |
| AM-101 | for treatment of tinnitus | GRIN1 |
| AM-101 | for treatment of tinnitus | GRIN2A |
| AM-101 | for treatment of tinnitus | GRIN2B |
| AM-101 | for treatment of tinnitus | GRIN2C |
| AM-101 | for treatment of tinnitus | GRIN2D |
| AM-101 | for treatment of tinnitus | GRIN3A |
| AM-101 | for treatment of tinnitus | GRIN3B |
| AM-103 | antiinflammatory agent | ALOX5AP |
| AM-152 | antiinflammatory agent, antifibrotic agent | LPAR1 |
| AM-211 | antiinflammatory agent, antiallergy agent | GPR44 |
| AM-461 | antiinflammatory agent | PTGDR |
| AM-803 | antiinflammatory agent | ALOX5AP |
| AMAP102 | antiinflammatory agent, DMARD | HTR2B |
| AMAP102 | antiinflammatory agent, DMARD | HTR2C |
| AMD-070 | antiviral agent, HIV | CXCR4 |
| ALS 2-0426 | antidiabetic | DPP4 |
| amibegron | antidepressant | ADRB3 |
| amifostine | radiation-protective agent | ALPPL2 |
| amiodarone | antiarrhytmic agent | ADRA1A |
| amiodarone | antiarrhytmic agent | ADRB1 |
| amiodarone | antiarrhytmic agent | KCNH2 |
| amisulpride | antipsychotic agent | DRD2 |
| amisulpride | antipsychotic agent | DRD3 |
| amitriptyline | analgesic | SLC6A2 |
| amitriptyline | analgesic | SLC6A4 |
| ketamine | analgesic | GRIN3A |
| amlodipine | antihypertensive agent, cardiovascular agent | CACNA1C |
| amlodipine | antihypertensive agent, cardiovascular agent | CACNA1D |
| amlodipine | antihypertensive agent, cardiovascular agent | CACNA1S |
| amlodipine | antihypertensive agent, cardiovascular agent | CACNA2D1 |
| amlodipine | antihypertensive agent, cardiovascular agent | CACNB2 |
| amonafide | antineoplastic agent | TOP2A |
| amonafide | antineoplastic agent | TOP2B |
| aliskiren | antihypertensive agent | REN |
| amlodipine | antihypertensive agent | CACNA1C |
| amlodipinc | antihypertensive agent | CACNA1D |
| amlodipine | antihypertensive agent | CACNA1S |
| amlodipine | antihypertensive agent | CACNA2D1 |
| amlodipine | antihypertensive agent | CACNB2 |
| hydrochlorothiazide | antihypertensive agent | SLC12A3 |
| AN-2728 | antiinflammatory agent, antipsoriatic | PDE4A |
| AN-2728 | antiinflammatory agent, antipsoriatic | PDE4B |
| AN-2898 | antiinflammatory agent, antipsoriatic | PDE4A |
| AN-2898 | antiinflammatory agent, antipsoriatic | PDE4B |
| ANA773 | antineoplastic agent | TLR7 |
| Anacetrapib | for treatment of dyslipidemia | CETP |
| anamorelin | appetite stimulating agent | GHSR |
| anastrozole | antineoplastic agent | CYP19A1 |
| anatibant | for treatment of traumatic brain injury | BDKRB2 |
| ANAVEX 2-73 | for treatment of Alzheimer's disease | SIGMAR1 |
| clomifene | for treatment of testosterone deficiency | ESR1 |
| anhydrovinblastin | antineoplastic agent | TUBB |
| docetaxel | antineoplastic agent | TUBB1 |
| AP1030 | antiobesity agent | MC1R |
| AP1030 | antiobesity agent | MC4R |
| oxybutynin | for treatment of overactive bladder | CHRM1 |
| oxybutynin | for treatment of overactive bladder | CHRM2 |
| oxybutynin | for treatment of overactive bladder | CHRM3 |
| APC-100 | antineoplastic agent | AR |
| APD125 | for treatment of insomnia | HTR2A |
| APD421 | antiemetic | DRD2 |
| APD668 | antidiabetic | GPR119 |
| APD791 | antithrombotic | HTR2A |
| APD916 | for treatment of narcolepsy | HRH3 |
| mepivacaine | anestethic | SCN10A |
| granisetron | antiemetic | HTR3A |
| apilimod | antiinflammatory agent, antipsoriatic | unknown |
| apixaban | antithrombotic | F10 |
| misoprostol | labor-inducing agent | PTGIR |
| Aplindore | antiparkinson agent, for treatment of restlegs legs | DRD2 |
| | syndrome | |
| apomorphine | for treatment of sexual dysfunction in | DRD2 |
| | women, for treatment of erectile | |
| | dysfunction, antiparkinson agent | |
| apomorphine | for treatment of sexual dysfunction in | DRD3 |
| | women, for treatment of erectile | |
| | dysfunction, antiparkinson agent | |
| apomorphine | for treatment of sexual dysfunction in | DRD4 |
| | women, for treatment of erectile | |
| | dysfunction, antiparkinson agent | |
| apomorphine | for treatment of sexual dysfunction in | DRD2 |
| | women, for treatment of erectile | |
| | dysfunction, antiparkinson agent | |
| apomorphine | for treatment of sexual dysfunction in | DRD3 |
| | women, for treatment of erectile | |
| | dysfunction, antiparkinson agent | |
| apomorphine | for treatment of sexual dysfunction in | DRD4 |
| | women, for treatment of erectile | |
| | dysfunction, antiparkinson agent | |
| apremilast | antiinflammatory agent, DMARD, antipsoriatic | PDE4A |
| apremilast | antiinflammatory agent, DMARD, antipsoriatic | PDE4B |
| aprepitant | antiemetic | TACR1 |
| apricoxib | antineoplastic agent | PTGS2 |
| AR-12 | antineoplastic agent | PDK1 |
| AR-12286 | for treatment of glaucoma | ROCK1 |
| AR-12286 | for treatment of glaucoma | ROCK2 |
| AR-42 | antineoplastic agent | HDAC1 |
| AR-42 | antineoplastic agent | HDAC10 |
| AR-42 | antineoplastic agent | HDAC11 |
| AR-42 | antineoplastic agent | HDAC2 |
| AR-42 | antineoplastic agent | HDAC3 |
| AR-42 | antineoplastic agent | HDAC4 |
| AR-42 | antineoplastic agent | HDAC5 |
| AR-42 | antineoplastic agent | HDAC6 |
| AR-42 | antineoplastic agent | HDAC7A |
| AR-42 | antineoplastic agent | HDAC8 |
| AR-42 | antineoplastic agent | HDAC9 |
| AR9281 | antihypertensive agent | EPHX1 |
| AR9281 | antihypertensive agent | EPHX2 |
| arbaclofen | symptomatic treatment for fragile X syndrome | GABBR1 |
| arbaclofen | symptomatic treatment for fragile X syndromc | GABBR2 |
| ARC100 | antineoplastic agent | TUBB1 |
| clonidine | for treatment of diabetic neuropathy, for | ADRA2A |
| | treatment of ADHD, antimucositic | |
| clonidine | for treatment of diabetic neuropathy, for | ADRA2B |
| | treatment of ADHD, antimucositic | |
| clonidine | for treatment of diabetic neuropathy, for | ADRA2C |
| | treatment of ADHD, antimucositic | |
| ARD-07 | for treatment of growth hormone deficiency | GHR |
| Argatroban | anticoagulant | F2 |
| ARI-2243 | antidiabetic | DPP4 |
| ARI-3037MO | Vitamin B analog, for treatment for | GPR109A |
| | hyperlipidemia | |
| ARI-3037MO | Vitamin B analog, for treatment for | GPR109B |
| | hyperlipidemia | |
| ARI-3037MO | Vitamin B analog, for treatment for | NNMT |
| | hyperlipidemia | |
| ARI-3037MO | Vitamin B analog, for treatment for | QPRT |
| | hyperlipidemia | |
| armodafinil | central nervous system stimulant | SLC6A3 |
| ARN-509 | antineoplastic agent | AR |
| ARQ-197 | antineoplastic agent | MET |
| ARQ-501 | antineoplastic agent | TOP1 |
| ARQ-621 | antineoplastic agent | KIF11 |
| ARRY-162 | antiinflammatory agent, DMARD, antincoplastic | MAP2K1 |
| | agent | |
| ARRY-162 | antiinflammatory agent, DMARD, antineoplastic | MAP2K2 |
| | agent | |
| ARRY-300 | antiinflammatory agent, DMARD, antineoplastic | MAP2K1 |
| | agent | |
| ARRY-300 | antiinflammatory agent, DMARD, antineoplastic | MAP2K2 |
| | agent | |
| ARRY-334543 | antineoplastic agent | EGFR |
| ARRY-334543 | antineoplastic agent | ERBB2 |
| ARRY-380 | antineoplastic agent | ERBB2 |
| ARRY-403 | antidiabetic | GCK |
| ARRY-614 | for treatment of myelodysplastic syndrome | ABL1 |
| ARRY-614 | for treatment of myelodysplastic syndrome | KDR |
| ARRY-614 | for treatment of myelodysplastic syndrome | MAPK11 |
| ARRY-614 | for treatment of myelodysplastic syndrome | MAPK12 |
| ARRY-614 | for treatment of myelodysplastic syndrome | MAPK13 |
| ARRY-614 | for treatment of myelodysplastic syndrome | MAPK14 |
| ARRY-614 | for treatment of myelodysplastic syndrome | TEK |
| ARRY-797 | antineoplastic agent | MAPK11 |
| ARRY-797 | antineoplastic agent | MAPK12 |
| ARRY-797 | antineoplastic agent | MAPK13 |
| ARRY-797 | antineoplastic agent | MAPK14 |
| arsenic trioxide | antineoplastic agent | CCND1 |
| arsenic trioxide | antineoplastic agent | IKBKB |
| arsenic trioxide | antincoplastic agent | JUN |
| arsenic trioxide | antineoplastic agent | MAPK1 |
| arsenic trioxide | antineoplastic agent | MAPK3 |
| arsenic trioxide | antineoplastic agent | TXNRD1 |
| arverapamil | for treatment of irritable bowel syndrome | CACNA1C |
| arverapamil | for treatment of irritable bowel syndrome | CACNA1D |
| arverapamil | for treatment of irritable bowel syndrome | CACNA1F |
| arverapamil | for treatment of irritable bowel syndrome | CACNA1G |
| arverapamil | for treatment of irritable bowel syndrome | CACNA1S |
| arverapamil | for treatment of irritable bowel syndrome | CACNB1 |
| arverapamil | for treatment of irritable bowel syndrome | CACNB2 |
| arverapamil | for treatment of irritable bowel syndrome | CACNB3 |
| arverapamil | for treatment of irritable bowel syndrome | CACNB4 |
| sufentanil | adjuvant to anesthesia | OPRM1 |
| sufentanil | adjuvant to anesthesia | OPRM1 |
| sufentanil | analgesic, sedative | OPRM1 |
| triazolam | analgesic, sedative | GABRA1 |
| triazolam | analgesic, sedative | GABRA2 |
| triazolam | analgesic, sedative | GABRA3 |
| triazolam | analgesic, sedative | GABRA4 |
| triazolam | analgesic, sedative | GABRA5 |
| triazolam | analgesic, sedative | GABRA6 |
| triazolam | analgesic, sedative | GABRB1 |
| triazolam | analgesic, sedative | GABRB2 |
| triazolam | analgesic, sedative | GABRB3 |
| triazolam | analgesic, sedative | GABRD |
| triazolam | analgesic, scdativc | GABRE |
| triazolam | analgesic, sedative | GABRG1 |
| triazolam | analgesic, sedative | GABRG2 |
| triazolam | analgesic, sedative | GABRG3 |
| triazolam | analgesic, sedative | GABRP |
| triazolam | analgesic, sedative | GABRQ |
| triazolam | analgesic, sedative | GABRR1 |
| triazolam | analgesic, sedative | GABRR2 |
| triazolam | analgesic, sedative | GABRR3 |
| Arzoxifene | antineoplastic agent, antiosteoporotic agent | ESR1 |
| ASC-J9 | dermatological agent | AR |
| Asenapine | antipsychotic agent | ADRA1A |
| Asenapine | antipsychotic agent | ADRA2A |
| Asenapine | antipsychotic agent | ADRA2B |
| Asenapine | antipsychotic agent | ADRA2C |
| Asenapine | antipsychotic agent | DRD1 |
| Asenapine | antipsychotic agent | DRD2 |
| Asenapine | antipsychotic agent | DRD3 |
| Asenapine | antipsychotic agent | DRD4 |
| Asenapine | antipsychotic agent | HRH1 |
| Asenapine | antipsychotic agent | HRH2 |
| Asenapine | antipsychotic agent | HTR1A |
| Asenapine | antipsychotic agent | HTR1B |
| Asenapine | antipsychotic agent | HTR2A |
| Asenapine | antipsychotic agent | HTR2B |
| Asenapine | antipsychotic agent | HTR2C |
| Asenapine | antipsychotic agent | HTR5A |
| Asenapine | antipsychotic agent | HTR6 |
| Asenapine | antipsychotic agent | HTR7 |
| asimadoline | analgesic | OPRK1 |
| ipragliflozin | antidiabetic | SLC5A2 |
| AT-101 | antineoplastic agent | BAD |
| AT-101 | antineoplastic agent | BCL2 |
| AT-101 | antineoplastic agent | MCL1 |
| AT13387 | antineoplastic agent | HSP90AA1 |
| AT13387 | antineoplastic agent | HSP90AB1 |
| fentanyl | analgesic | OPRD1 |
| fentanyl | analgesic | OPRD1 |
| fentanyl | analgesic | OPRM1 |
| fentanyl | analgesic, opioid | OPRM1 |
| AT7519 | antineoplastic agent | CDK2 |
| AT9283 | antineoplastic agent | AURKA |
| AT9283 | antineoplastic agent | AURKB |
| atamestane | antineoplastic agent | CYP19A1 |
| toremifene | antineoplastic agent | ESR1 |
| toremifene | antineoplastic agent | ESR2 |
| ATHX-105 | antiobesity agent | HTR2C |
| docetaxel | antineoplastic agent | TUBB1 |
| ATI-7505 | Parasympathomimetic | HTR4 |
| prednisone | antiinflammatory agent, corticosteroid | NR3C1 |
| atomoxetine | for treatment of ADHD | SLC6A2 |
| atorvastatin | antihypecholesterolemic agent | HMGCR |
| atrasentan | antineoplastic agent | EDNRA |
| AUS-131 | for treatment of menopausal symtpoms | ESR2 |
| AV-412 | antineoplastic agent | EGFR |
| AV-412 | antineoplastic agent | ERBB2 |
| AV608 | antidepressant, for treatment of irritable bowel | TACR1 |
| | syndrome, antispasmodic | |
| tivozanib | antineoplastic agent | FLT1 |
| tivozanib | antineoplastic agent | FLT4 |
| tivozanib | antineoplastic agent | KDR |
| Avanafil | for treatment of erectile dysfunction | PDE5A |
| AVE-1625 | antiobesity agent, for treatment for Alzheimer's | CNR1 |
| | disease | |
| phentolamine | for treatment of erectile dysfunction | ADRA1A |
| phentolamine | for treatment of erectile dysfunction | ADRA2A |
| AVL-292 | antineoplastic agent | BTK |
| AVN-101 | for treatment of alzheimer's disease | HTR6 |
| AVN-211 | antipsychotic agent | HTR6 |
| AVN-322 | for treatment of alzheimer's disease | HTR6 |
| AVN-944 | antineoplastic agent | IMPDH1 |
| AVN-944 | antincoplastic agent | IMPDH2 |
| avosentan | antihypertensive agent | EDNRA |
| dextromethorphan | antitussive agent | GRIN3A |
| dextromethorphan | antitussive agent | SIGMAR1 |
| axitinib | antineoplastic agent | FLT1 |
| axitinib | antineoplastic agent | FLT4 |
| axitinib | antineoplastic agent | KDR |
| axitinib | antineoplastic agent | KIT |
| axitinib | antineoplastic agent | PDGFRA |
| axitinib | antineoplastic agent | PDGFRB |
| AXL1717 | antineoplastic agent | IGF1R |
| prochlorperazine | antimigraine agent | DRD2 |
| alprazolam | anxiolytic, sedative, hypnotic | GABRA1 |
| alprazolam | anxiolytic, sedative, hypnotic | GABRA2 |
| alprazolam | anxiolytic, sedative, hypnotic | GABRA3 |
| alprazolam | anxiolytic, sedative, hypnotic | GABRA4 |
| alprazolam | anxiolytic, sedative, hypnotic | GABRA5 |
| alprazolam | anxiolytic, sedative, hypnotic | GABRA6 |
| alprazolam | anxiolytic, sedative, hypnotic | GABRB1 |
| alprazolam | anxiolytic, sedative, hypnotic | GABRB2 |
| alprazolam | anxiolytic, sedative, hypnotic | GABRB3 |
| alprazolam | anxiolytic, sedative, hypnotic | GABRD |
| alprazolam | anxiolytic, sedative, hypnotic | GABRE |
| alprazolam | anxiolytic, sedative, hypnotic | GABRG1 |
| alprazolam | anxiolytic, sedative, hypnotic | GABRG2 |
| alprazolam | anxiolytic, sedative, hypnotic | GABRG3 |
| alprazolam | anxiolytic, sedative, hypnotic | GABRP |
| alprazolam | anxiolytic, sedative, hypnotic | GABRQ |
| alprazolam | anxiolytic, sedative, hypnotic | GABRR1 |
| alprazolam | anxiolytic, sedative, hypnotic | GABRR2 |
| alprazolam | anxiolytic, sedative, hypnotic | GABRR3 |
| fentanyl | adjuvant to anesthesia | OPRD1 |
| fentanyl | adjuvant to anesthesia | OPRM1 |
| loxapine | antipsychotic agent | DRD2 |
| loxapine | antipsychotic agent | HTR2A |
| zaleplon | hypnotic | GABRA1 |
| zaleplon | hypnotic | TSPO |
| azacitidine | antineoplastic agent | DNMT1 |
| AZD-0837 | anticoagulant | F2 |
| AZD2066 | analgesic, for treatment of gastroesophageal | GRM5 |
| | reflux disease | |
| AZD6244, ARRY-142886 | antineoplastic agent | MAP2K1 |
| AZD6244, ARRY-142886 | antineoplastic agent | MAP2K2 |
| AZD-8330 | antineoplastic agent | MAP2K1 |
| AZD-8848 | antiallergy agent | TLR7 |
| azelastine | antiallergy agent | HRH1 |
| azelastine | antiallergy agent | HRH1 |
| azilsartan | antihypertensive agent | AGTR1 |
| balsalazide | antiinflammatory agent | ALOX5 |
| balsalazide | antiinflammatory agent | PPARG |
| balsalazide | antiinflammatory agent | PTGS1 |
| balsalazide | antiinflammatory agent | PTGS2 |
| bardoxolone | antineoplastic agent | NFKB1 |
| bazedoxifene | antiosteoporotic agent | ESR1 |
| bazedoxifene | antiosteoporotic agent | ESR2 |
| ulodesine | antiinflammatory agent | PNP |
| becatecarin | antineoplastic agent | TOP2A |
| becatecarin | antineoplastic agent | TOP2B |
| beclomethasone | antiinflammatory agent, glucocorticoid | NR3C1 |
| beclomethasone | antiinflammatory agent, glucocorticoid | NR3C1 |
| beclomethasone | antiinflammatory agent, glucocorticoid | NR3C1 |
| buprenorphine | antidepressant, analgesic, for treatment of opioid | OPRK1 |
| | addiction | |
| buprenorphine | antidepressant, analgesic, for treatment of opioid | OPRM1 |
| | addiction | |
| fentanyl | analgesic | OPRD1 |
| fentanyl | analgesic | OPRM1 |
| benazepril | antihypertensive agent | ACE |
| bepotastine | antiallergy agent | HRH1 |
| beraprost | antihypertensive agent | PTGIR |
| betamethasone | antiinflammatory agent, glucocorticoid | NR3C1 |
| betamethasone | antiinflammatory agent, glucocorticoid | NR3C1 |
| betrixaban | antithrombotic | F10 |
| boxarotenc | antincoplastic agent | RXRA |
| bexarotene | antineoplastic agent | RXRB |
| bexarotene | antineoplastic agent | RXRG |
| BF-1 | antimigraine agent | HTR2B |
| BF-Derm1 | antiallergy agent | HDC |
| BG-9928 | for treatment of congestive heart failure | ADORA1 |
| fluoxetine | for treatment of sleep apnea | SLC6A4 |
| ondansetron | for treatment of sleep apnea | HTR3A |
| BGC20-1531 | antimigraine agent | PTGER4 |
| BGG-492 | anticonvulsant, antimigraine agent | GRIA1 |
| BGG-492 | anticonvulsant, antimigraine agent | GRIA2 |
| BGG-492 | anticonvulsant, antimigraine agent | GRIA3 |
| BGG-492 | anticonvulsant, antimigraine agent | GRIA4 |
| progesterone | neuroprotectant for stroke victims | ESR1 |
| progesterone | neuroprotectant for stroke victims | NR3C2 |
| progesterone | neuroprotectant for stroke victims | PGR |
| BI-10773 | antidiabetic | SLC5A2 |
| olodaterol | bronchodilator | ADRB2 |
| Nintedanib | antineoplastic agent | FGFR1 |
| Nintedanib | antineoplastic agent | FGFR2 |
| Nintedanib | antineoplastic agent | FGFR3 |
| Nintedanib | antineoplastic agent | FLT1 |
| Nintedanib | antineoplastic agent | FLT4 |
| Nintedanib | antineoplastic agent | KDR |
| Nintedanib | antineoplastic agent | PDGFRA |
| Nintedanib | antineoplastic agent | PDGFRB |
| Bicalutamide | antineoplastic agent | AR |
| bifeprunox | antipsychotic agent, antiparkinson agent | DRD2 |
| bifeprunox | antipsychotic agent, antiparkinson agent | DRD3 |
| bifeprunox | antipsychotic agent, antiparkinson agent | HTR1A |
| bifeprunox | antipsychotic agent, antiparkinson agent | HTR2A |
| bifeprunox | antipsychotic agent, antiparkinson agent | HTR2C |
| bifeprunox | antipsychotic agent, antiparkinson agent | HTR7 |
| BIM23A760 | antineoplastic agent, treatment for acromegaly | DRD2 |
| BIM23A760 | antineoplastic agent, treatment for acromegaly | SSTR2 |
| BIM23A760 | antineoplastic agent, treatment for acromegaly | SSTR5 |
| bimatoprost | antiglaucomic agent | PTGER1 |
| bimatoprost | antiglaucomic agent | PTGER3 |
| bimatoprost | antiglaucomic agent | PTGFR |
| bimoclomol | for treatment of diabetic neuropathy | HSF1 |
| bimosiamose | antiinflammatory agent, antipsoriatic | SELE |
| bimosiamose | antiinflammatory agent, antipsoriatic | SELL |
| bimosiamose | antiinflammatory agent, antipsoriatic | SELP |
| docetaxel | antineoplastic agent | BCL2 |
| docetaxel | antineoplastic agent | TUBB1 |
| binodenoson | diagnostic agent | ADORA2A |
| estradiol | hormone replacement, treatment for menopause | ESR1 |
| estradiol | hormone replacement, treatment for menopause | ESR2 |
| testosterone | hormone replacement | AR |
| dapagliflozin | antidiabetic | SLC5A2 |
| BMS-582949 | antiinflammatory agent, DMARD, antipsoriatic | MAPK11 |
| BMS-582949 | antiinflammatory agent, DMARD, antipsoriatic | MAPK12 |
| BMS-582949 | antiinflammatory agent, DMARD, antipsoriatic | MAPK13 |
| BMS-582949 | antiinflammatory agent, DMARD, antipsoriatic | MAPK14 |
| BMS-299897 | for treatment of alzheimer's disease | APH1A |
| BMS-299897 | for treatment of alzheimer's disease | APH1B |
| BMS-299897 | for treatment of alzheimer's disease | NCSTN |
| BMS-299897 | for treatment of alzheimer's disease | PSEN1 |
| BMS-299897 | for treatment of alzheimer's disease | PSEN2 |
| BMS-299897 | for treatment of alzheimer's disease | PSENEN |
| BMS-708163 | for treatment of alzheimer's disease | APH1A |
| BMS-708163 | for treatment of alzheimer's disease | APH1B |
| BMS-708163 | for treatment of alzheimer's disease | NCSTN |
| BMS-708163 | for treatment of alzheimer's disease | PSEN1 |
| BMS-708163 | for treatment of alzheimer's disease | PSEN2 |
| BMS-708163 | for treatment of alzheimer's disease | PSENEN |
| BMS-754807 | antineoplastic agent | IGF1R |
| BMS-863233 | antineoplastic agent | CDC7 |
| calcitonin | antiosteoporotic agent | CALCR |
| NCX116 | for treatment of glaucoma | PTGFR |
| bosutinib | antineoplastic agent | ABL1 |
| bosutinib | antineoplastic agent | SRC |
| brimonidine | for treatment of glaucoma | ADRA2A |
| brimonidine | for treatment of glaucoma | ADRA2A |
| timolol | for treatment of glaucoma | ADRB1 |
| timolol | for treatment of glaucoma | ADRB2 |
| Brivaracetam | anticonvulsant | SV2A |
| bromfenac | opthalmological agent, NSAID | PTGS1 |
| bromfenac | opthalmological agent, NSAID | PTGS2 |
| bromocriptine | antidiabetic | DRD2 |
| bromocriptine | antidiabetic | DRD3 |
| Bryostatin | for treatment of alzheimer's disease | PRKCA |
| Bryostatin | for treatment of alzheimer's disease | PRKCB |
| Bryostatin | for treatment of alzheimer's disease | PRKCD |
| Bryostatin | for treatment of alzheimer's disease | PRKCE |
| Bryostatin | for treatment of alzheimer's disease | PRKCG |
| Bryostatin | for treatment of alzheimer's disease | PRKCH |
| Bryostatin | for treatment of alzheimer's disease | PRKCQ |
| Bryostatin | for treatment of alzheimer's disease | PRKD1 |
| Bryostatin | for treatment of alzheimer's disease | PRKD2 |
| Bryostatin | for treatment of alzheimer's disease | PRKD3 |
| Bryostatin-1 | antineoplastic agent | PRKCA |
| Bryostatin-1 | antineoplastic agent | PRKCB |
| Bryostatin-1 | antineoplastic agent | PRKCD |
| Bryostatin-1 | antineoplastic agent | PRKCE |
| Bryostatin-1 | antineoplastic agent | PRKCG |
| Bryostatin-1 | antineoplastic agent | PRKCH |
| Bryostatin-1 | antineoplastic agent | PRKCQ |
| Bryostatin-1 | antineoplastic agent | PRKD1 |
| Bryostatin-1 | antineoplastic agent | PRKD2 |
| Bryostatin-1 | antineoplastic agent | PRKD3 |
| fentanyl | analgesic | OPRD1 |
| fentanyl | analgesic | OPRM1 |
| prochlorperazine | antiemetic | DRD2 |
| bucindolol | for treatment of heart failure | ADRB1 |
| bucindolol | for treatment of heart failure | ADRB2 |
| budesonide | antiinflammatory agent, glucocorticoid | NR3C1 |
| Formoterol | bronchodilator | ADRB2 |
| budesonide | antiinflammatory agent, glucocorticoid | NR3C1 |
| budesonide | antiinflammatory agent, glucocorticoid | NR3C1 |
| budesonide | antiinflammatory agent, glucocorticoid | NR3C1 |
| budesonide | antiinflammatory agent, glucocorticoid | NR3C1 |
| budesonide | antiinflammatory agent, glucocorticoid | NR3C1 |
| budesonide | antiinflammatory agent, glucocorticoid | NR3C1 |
| budiodarone | antiarrhytmic agent | ADRB1 |
| budiodarone | antiarrhytmic agent | CACNA2D2 |
| budiodarone | antiarrhytmic agent | KCNH2 |
| buprenorphine | antidepressant, analgesic, for treatment of opioid | OPRK1 |
| | addiction | |
| buprenorphine | antidepressant, analgesic, for treatment of opioid | OPRM1 |
| | addiction | |
| naloxone | analgesic | OPRK1 |
| naloxone | analgesic | OPRM1 |
| buprenorphine | antidepressant, analgesic, for treatment of opioid | OPRK1 |
| | addiction | |
| buprenorphine | antidepressant, analgesic, for treatment of opioid | OPRM1 |
| | addiction | |
| naloxone | for treatment of opioid addiction | OPRK1 |
| naloxone | for treatment of opioid addiction | OPRM1 |
| buprenorphine | antidepressant, analgesic, for treatment of opioid | OPRK1 |
| | addiction | |
| buprenorphine | antidepressant, analgesic, for treatment of opioid | OPRM1 |
| | addiction | |
| buprenorphine | antidepressant, analgesic, for treatment of opioid | OPRK1 |
| | addiction | |
| buprenorphine | antidepressant, analgesic, for treatment of opioid | OPRM1 |
| | addiction | |
| buprenorphine | antidepressant, analgesic, for treatment of opioid | OPRK1 |
| | addiction | |
| buprenorphine | antidepressant, analgesic, for treatment of opioid | OPRM1 |
| | addiction | |
| bupropion | antidepressant, appetite suppressant, smoking- | SLC6A2 |
| | cessation agent | |
| bupropion | antidepressant, appetite suppressant, smoking- | SLC6A3 |
| | cessation agent | |
| BVT.115959 | analgesic | ADORA2A |
| BVT.28949 | for treatment of glaucoma | HTR2A |
| amphetamine | for treatment of cognitive dysfunction, for | CARTPT |
| | treatment of ADHD | |
| amphetamine | for treatment of cognitive dysfunction, for | SLC18A2 |
| | treatment of ADHD | |
| amphetamine | for treatment of cognitive dysfunction, for | SLC6A3 |
| | treatment of ADHD | |
| amphetamine | for treatment of cognitive dysfunction, for | TAAR1 |
| | treatment of ADHD | |
| C-1311 | antineoplastic agent | TOP1 |
| C-1311 | antineoplastic agent | TOP2A |
| cabazitaxel | antineoplastic agent | TUBA4A |
| cabazitaxel | antineoplastic agent | TUBB1 |
| amlodipine | antihypertensive agent, cardiovascular agent | CACNA1C |
| amlodipine | antihypertensive agent, cardiovascular agent | CACNA1D |
| amlodipine | antihypertensive agent, cardiovascular agent | CACNA1S |
| amlodipine | antihypertensive agent, cardiovascular agent | CACNA2D1 |
| amlodipine | antihypertensive agent, cardiovascular agent | CACNB2 |
| atorvastatin | anticholesterolaemic agent | HMGCR |
| CAL-101 | antineoplastic agent | PIK3CD |
| betamethasone | antiinflammatory agent, glucocorticoid | NR3C1 |
| calcipotriene | antipsoriatic agent | VDR |
| calcitriol | antipsoriatic agent | VDR |
| buprenorphine | antidepressant, analgesic, for treatment of opioid | OPRK1 |
| | addiction | |
| buprenorphine | antidepressant, analgesic, for treatment of opioid | OPRM1 |
| | addiction | |
| Canagliflozin | antidiabetic | SLC5A2 |
| candesartan | antihypertensive agent | AGTR1 |
| cangrelor | antithrombotic | P2RY12 |
| PRS-211375 | analgesic | CNR2 |
| CAP7.1 | antineoplastic agent | TOP2A |
| Caprospinol | for treatment of alzheimer's disease | APP |
| Carfilzomib | antineoplastic agent | PSMB1 |
| Carfilzomib | antineoplastic agent | PSMB2 |
| Carfilzomib | antineoplastic agent | PSMB5 |
| cariprazine | antipsychotic agent | DRD2 |
| cariprazine | antipsychotic agent | DRD3 |
| carvedilol | for treatment of congestive heart failure | ADRA1A |
| carvedilol | cardiovascular agent | ADRB1 |
| carvedilol | cardiovascular agent | ADRB2 |
| Casopitant | antiemetic | TACR1 |
| dronabinol | analgesic | CNR1 |
| dronabinol | analgesic | CNR2 |
| CB-03-01 | dermatological agent | AR |
| caricotamide | antineoplastic agent | NQ02 |
| tretazicar | antineoplastic agent | DNA |
| abiraterone | antineoplastic agent | CYP17A1 |
| JNK-401 | antineoplastic agent | MAPK10 |
| JNK-401 | antineoplastic agent | MAPK8 |
| JNK-401 | antineoplastic agent | MAPK9 |
| CCX025 | antiinflammatory agent | CCR9 |
| CCX140 | antiinflammatory agent, antidiabetic | CCR2 |
| CCX168 | antiinflammatory agent, for treatment for | C5AR1 |
| | autoimmune disease | |
| CCX282 | antiinflammatory agent, for treatment of Chron's | CCR9 |
| | disease, for treatment of ulceraite colitis | |
| CCX354 | antiinflammatory agent, DMARD | CCR1 |
| CCX832 | antiinflammatory agent, for treatment for | CMKLR1 |
| | autoimmune disease | |
| fenofibrate | anticholesterolaemic agent | PPARA |
| azelastine | antiallergy agent | HRH1 |
| budesonide | antiinflammatory agent, glucocorticoid | NR3C1 |
| cediranib | antineoplastic agent | FLT1 |
| cediranib | antineoplastic agent | FLT4 |
| cediranib | antineoplastic agent | KDR |
| celecoxib | NSAID | PTGS2 |
| mycophenolate mofetil | immunosuppressant | IMPDH1 |
| mycophenolate mofetil | immunosuppressant | IMPDH2 |
| synthetic conjugated estrogens | for treatment of postmenopausal symptoms | ESR1 |
| synthetic conjugated estrogens | for treatment of postmenopausal symptoms | ESR2 |
| histamine | cytorprotective agent during cancer treatment | HRH2 |
| CER-002 | cardiovascular agent | PPARD |
| acetylsalicylic acid | NSAID | PTGS1 |
| acetylsalicylic acid | NSAID | PTGS2 |
| niacin | antidyslipidacmic agent | GPR109A |
| niacin | antidyslipidaemic agent | GPR109B |
| niacin | antidyslipidaemic agent | NNMT |
| niacin | antidyslipidaemic agent | QPRT |
| diclofenac | NSAID | PTGS1 |
| diclofenac | NSAID | PTGS2 |
| cetilistat | antiobesity agent | PNLIP |
| cetirizine | antiallergy agent | HRH1 |
| CF-101 | antiinflammatory agent, DMARD | ADORA3 |
| CF-102 | antineoplastic agent | ADORA3 |
| CG100649 | NSAID | CA1 |
| CG100649 | NSAID | PTGS2 |
| clopidogrel | antiplatelet agent | P2RY12 |
| omeprazol | antiulcer agent | ATP4A |
| CH-1504 | antiinflammatory agent, DMARD | DHFR |
| CHF 4227 | antiosteoporotic agent | ESR1 |
| CHF 4227 | antiosteoporotic agent | ESR2 |
| beclomethasone | antiinflammatory agent, glucocorticoid | NR3C1 |
| formoterol | antiasthmatic agent | ADRB2 |
| chidamide | antineoplastic agent | HDAC1 |
| chidamide | antineoplastic agent | HDAC10 |
| chidamide | antineoplastic agent | HDAC2 |
| chidamide | antineoplastic agent | HDAC3 |
| CHIR-265 | antineoplastic agent | BRAF |
| CHIR-265 | antineoplastic agent | KDR |
| CHIR-265 | antineoplastic agent | RAF1 |
| cyclosporine | immunosuppressant | CAMLG |
| cyclosporine | immunosuppressant | PPP3R2 |
| tadalafil | for treatment of erectile dysfunction | PDE5A |
| cilansetron | for treatment of irritable bowel syndrome | HTR3A |
| cimicoxib | NSAID | PTGS2 |
| isotretinoin | for treatment of acne | RARA |
| escitalopram | antidepressant | SLC6A4 |
| tiramsetiv | for treatment of skeletal muscle disorders | TNNC1 |
| | associated with aging and neuro-degenerative | |
| | disorders. | |
| tiramsetiv | for treatment of skeletal muscle disorders | TNNC2 |
| | associated with aging and neuro-degenerative | |
| | disorders. | |
| tiramsetiv | for treatment of skeletal muscle disorders | TNNI1 |
| | associated with aging and neuro-degenerative | |
| | disorders. | |
| tiramsetiv | for treatment of skeletal muscle disorders | TNNI2 |
| | associated with aging and neuro-degenerative | |
| | disorders. | |
| tiramsetiv | for treatment of skeletal muscle disorders | TNNT1 |
| | associated with aging and neuro-degenerative | |
| | disorders. | |
| tiramsetiv | for treatment of skeletal muscle disorders | TNNT2 |
| | associated with aging and neuro-degenerative | |
| | disorders. | |
| clazosentan | for treatment and prevention of vasospasm | EDNRA |
| clevidipine | antihypertensive agent | CACNA1C |
| clevidipine | antihypertensive agent | CACNA1D |
| clevidipine | antihypertensive agent | CACNA1F |
| clevidipine | antihypertensive agent | CACNA1S |
| clobazam | anxiolytic, anticonvulsant | GABRA1 |
| clobazam | anxiolytic, anticonvulsant | GABRA2 |
| clobazam | anxiolytic, anticonvulsant | GABRA3 |
| clobazam | anxiolytic, anticonvulsant | GABRA4 |
| clobazam | anxiolytic, anticonvulsant | GABRA5 |
| clobazam | anxiolytic, anticonvulsant | GABRA6 |
| clobazam | anxiolytic, anticonvulsant | GABRB1 |
| clobazam | anxiolytic, anticonvulsant | GABRB2 |
| clobazam | anxiolytic, anticonvulsant | GABRB3 |
| clobazam | anxiolytic, anticonvulsant | GABRD |
| clobazam | anxiolytic, anticonvulsant | GABRE |
| clobazam | anxiolytic, anticonvulsant | GABRG1 |
| clobazam | anxiolytic, anticonvulsant | GABRG2 |
| clobazam | anxiolytic, anticonvulsant | GABRG3 |
| clobazam | anxiolytic, anticonvulsant | GABRP |
| clobazam | anxiolytic, anticonvulsant | GABRQ |
| clobazam | anxiolytic, anticonvulsant | GABRR1 |
| clobazam | anxiolytic, anticonvulsant | GABRR2 |
| clobazam | anxiolytic, anticonvulsant | GABRR3 |
| clobetasol | antiinflammatory agent, corticosteroid | NR3C1 |
| clodronate | antineoplastic agent | SLC25A4 |
| clodronato | antincoplastic agent | SLC25A5 |
| clodronate | antineoplastic agent | SLC25A6 |
| Clofarabine | antineoplastic agent | POLA1 |
| Clofarabine | antineoplastic agent | RRM1 |
| clonidine | for treatment of diabetic neuropathy, for | ADRA2A |
| | treatment of ADHD, antimucositic | |
| clonidine | for treatment of diabetic neuropathy, for | ADRA2B |
| | treatment of ADHD, antimucositic | |
| clonidine | for treatment of diabetic neuropathy, for | ADRA2C |
| | treatment of ADHD, antimucositic | |
| clonidine | for treatment of diabetic neuropathy, for | ADRA2A |
| | treatment of ADHD, antimucositic | |
| clonidine | for treatment of diabetic neuropathy, for | ADRA2B |
| | treatment of ADHD, antimucositic | |
| clonidine | for treatment of diabetic neuropathy, for | ADRA2C |
| | treatment of ADHD, antimucositic | |
| CLX-0921 | antidiabetic | PPARG |
| CM2489 | antiinflammatory agent, antipsoriatic | ORA1 |
| CNDO101 | antineoplastic agent | TOP2A |
| CNF1010 | antineoplastic agent | HSP90AA1 |
| CNF1010 | antineoplastic agent | HSP90AB1 |
| CNS-5161 | analgesic | GRIN1 |
| CNS-5161 | analgesic | GRIN2A |
| CNS-5161 | analgesic | GRIN2B |
| CNS-5161 | analgesic | GRIN2C |
| CNS-5161 | analgesic | GRIN2D |
| CNS-5161 | analgesic | GRIN3A |
| CNS-5161 | analgesic | GRIN3B |
| CNS-7056 | sedative | GABRA2 |
| CNS-7056 | sedative | GABRA3 |
| CNS-7056 | sedative | GABRA5 |
| CNS-7056 | sedative | GABRA6 |
| CNS-7056 | sedative | GABRB1 |
| CNS-7056 | sedative | GABRB1 |
| CNS-7056 | sedative | GABRB2 |
| CNS-7056 | sedative | GABRB2 |
| CNS-7056 | sedative | GABRB3 |
| CNS-7056 | sedative | GABRD |
| CNS-7056 | scdative | GABRD |
| CNS-7056 | sedative | GABRE |
| CNS-7056 | sedative | GABRG1 |
| CNS-7056 | sedative | GABRG2 |
| CNS-7056 | sedative | GABRG3 |
| CNS-7056 | sedative | GABRG3 |
| CNS-7056 | sedative | GABRP |
| CNS-7056 | sedative | GABRQ |
| CNS-7056 | sedative | GABRR2 |
| CNV2197944 | analgesic | CACNA1B |
| oxycodone | analgesic | OPRD1 |
| oxycodone | analgesic | OPRK1 |
| oxycodone | analgesic | OPRM1 |
| oxycodone | analgesic | OPRD1 |
| oxycodone | analgesic | OPRK1 |
| oxycodone | analgesic | OPRM1 |
| COL-3 | antineoplastic agent | MMP2 |
| COL-3 | antineoplastic agent | MMP9 |
| colchicine | for treatment of gout | TUBB |
| bupivacaine | local anestethic, analgesic, neuralgia | SCN10A |
| conivaptan | for treatment of hyponatremia | AVPR1A |
| conivaptan | for treatment of hyponatremia | AVPR2 |
| estrogen | for symptomatic treatment of menopausal | ESR1 |
| | symptoms | |
| estrogen | for symptomatic treatment of menopausal | ESR2 |
| | symptoms | |
| progesterone | for symptomatic treatment of menopausal | ESR1 |
| | symptoms | |
| progesterone | for symptomatic treatment of menopausal | NR3C2 |
| | symptoms | |
| progesterone | for symptomatic treatment of menopausal | PGR |
| | symptoms | |
| ethinyl estradiol | contraceptive | ESR1 |
| gestodene | contraceptive | PGR |
| bupropion | antidepressant, appetite suppressant, smoking- | SLC6A2 |
| | cessation agent | |
| bupropion | antidepressant, appetite suppressant, smoking- | SLC6A3 |
| | cessation agent | |
| naltrexone | appetite suppressant | OPRD1 |
| naltrexone | appetite suppressant | OPRK1 |
| naltrexone | appetite suppressant | OPRM1 |
| fomepizole | for treatment of ethanol intolerance | ADH1A |
| fomepizole | for treatment of ethanol intolerance | ADH1B |
| fomepizole | for treatment of ethanol intolerance | ADH1C |
| cordycepin | antineoplastic agent | DNTT |
| CORT 108297 | for prevention of weight gain during | NR3C1 |
| | antipsychotic treatment | |
| CP-4126 | antineoplastic agent | DNA |
| CP-609,754 | antineoplastic agent | FNTA |
| CP-609,754 | antineoplastic agent | FNTB |
| CPG 10101 | immunostimulant | TLR9 |
| CPG 52364 | antiinflammatory agent | TLR7 |
| CPG 52364 | antiinflammatory agent | TLR8 |
| CPG 52364 | antiinflammatory agent | TLR9 |
| CPI-613 | antineoplastic agent | PDHA1 |
| CPI-613 | antineoplastic agent | PDHA2 |
| CPI-613 | antineoplastic agent | PDHB |
| CPI-613 | antineoplastic agent | PDK1 |
| CPI-613 | antineoplastic agent | PDK2 |
| CPI-613 | antineoplastic agent | PDK3 |
| CPI-613 | antineoplastic agent | PDK4 |
| semapimod | antiinflammatory agent, for treatment of Chron's | MAPK11 |
| | disease | |
| semapimod | antiinflammatory agent, for treatment of Chron's | MAPK12 |
| | disease | |
| semapimod | antiinflammatory agent, for treatment of Chron's | MAPK13 |
| | disease | |
| semapimod | antiinflammatory agent, for treatment of Chron's | MAPK14 |
| | disease | |
| floxuridine | antineoplastic agent | TYMS |
| irinotecan | antineoplastic agent | TOP1 |
| irinotecan | antineoplastic agent | TOP1MT |
| cytarabine | antineoplastic agent | POLB |
| daunorubicin | antineoplastic agent | TOP2A |
| daunorubicin | antineoplastic agent | TOP2B |
| CR665 | analgesic | OPRK1 |
| CR845 | analgesic | OPRK1 |
| pravastatin | antihypecholesterolemic agent | HMGCR |
| rosuvastatin | antihypecholesterolemic agent | HMGCR |
| 561679 | antidepressant | CRHR1 |
| crizotinib | antineoplastic agent | ALK |
| crizotinib | antineoplastic agent | MET |
| CRTH2 receptor antagonist | antiallergy agent | GPR44 |
| prednisolone | antiinflammatory agent, corticosteroid | NR3C1 |
| dipyridamole | anticoagulant | ADA |
| dipyridamole | anticoagulant | PDE10A |
| dipyridamole | anticoagulant | PDE4A |
| dipyridamole | anticoagulant | PDE5A |
| amoxapine | antidepressant | SLC6A2 |
| amoxapine | antidepressant | SLC6A4 |
| prednisolone | antiinflammatory agent, corticosteroid | NR3C1 |
| paroxetine | antidepressant | SLC6A4 |
| prednisolone | antiinflammatory agent, corticosteroid | NR3C1 |
| amoxapine | antidepressant | SLC6A2 |
| amoxapine | antidepressant | SLC6A4 |
| dipyridamole | antithrombotic | ADA |
| dipyridamole | antithrombotic | PDE10A |
| dipyridamole | antithrombotic | PDE4A |
| dipyridamole | antithrombotic | PDE5A |
| budesonide | antiinflammatory agent, glucocorticoid | NR3C1 |
| nortriptyline | antiasthmatic agent | SLC6A2 |
| nortriptyline | antiasthmatic agent | SLC6A4 |
| mometasone | antiinflammatory agent, glucocorticoid | NR3C1 |
| nortriptyline | antidepressant | SLC6A2 |
| nortriptyline | antidepressant | SLC6A4 |
| bezafibrate | antidiabetic | PPARA |
| diflunisal | antidiabetic | PTGS1 |
| diflunisal | antidiabetic | PTGS2 |
| CS-3030 | anticoagulant | F10 |
| CS-7017 | antineoplastic agent | PPARG |
| amlodipine | antihypertensive agent | CACNA1C |
| amlodipine | antihypertensive agent | CACNA1D |
| amlodipine | antihypertensive agent | CACNA1S |
| amlodipine | antihypertensive agent | CACNA2D1 |
| amlodipine | antihypertensive agent | CACNB2 |
| olmesartan | antihypertensive agent | AGTR1 |
| CTA018 | antiinflammatory agent, antipsoriatic | CYP24A1 |
| CTS-21166 | for treatment of Alzheimer's disease | BACE1 |
| CUDC-101 | antineoplastic agent | EGFR |
| CUDC-101 | antineoplastic agent | ERBB2 |
| CUDC-101 | antineoplastic agent | HDAC1 |
| CUDC-101 | antineoplastic agent | HDAC10 |
| CUDC-101 | antineoplastic agent | HDAC11 |
| CUDC-101 | antineoplastic agent | HDAC2 |
| CUDC-101 | antineoplastic agent | HDAC3 |
| CUDC-101 | antineoplastic agent | HDAC4 |
| CUDC-101 | antineoplastic agent | HDAC5 |
| CUDC-101 | antineoplastic agent | HDAC6 |
| CUDC-101 | antineoplastic agent | HDAC7 |
| CUDC-101 | antineoplastic agent | HDAC8 |
| CUDC-101 | antineoplastic agent | HDAC9 |
| CVT-3619 | antihyperlipidemic agent | ADORA1 |
| CVT-6883 | antiasthmatic agent | ADORA2B |
| CX157 | antidepressant | MAOA |
| CX1632/S 47445 | for treatment of Alzheimer's disease | GRIA1 |
| CX1632/S 47445 | for treatment of Alzheimer's disease | GRIA2 |
| CX1632/S 47445 | for treatment of Alzheimer's disease | GRIA3 |
| CX1632/S 47445 | for treatment of Alzheimer's disease | GRIA4 |
| CX-4945 | antineoplastic agent | CSNK2A1 |
| CX717 | for treatment of Alzheimer's disease | GRIA1 |
| CX717 | for treatment of Alzheimer's disease | GRIA2 |
| CX717 | for treatment of Alzheimer's disease | GRIA3 |
| CX717 | for treatment of Alzheimer's disease | GRIA4 |
| CXB909 | for treatment of chemotherapy-induced | LNGFR |
| | peripheral neuropathy | |
| CXB909 | for treatment of chemotherapy-induced | NTRK1 |
| | peripheral neuropathy | |
| CYC116 | antineoplastic agent | AURKA |
| CYC116 | antineoplastic agent | AURKB |
| CYC116 | antineoplastic agent | KDR |
| cyclosporine | immunosuppressant | CAMLG |
| cyclosporine | immunosuppressant | PPP3R2 |
| duloxetine | antidepressant | SLC6A2 |
| duloxetine | antidepressant | SLC6A4 |
| cysteamine | for treatment of corneal cystine accumulation | cystine |
| cytarabine | antineoplastic agent | POLB |
| D3263 | antineoplastic agent | TRPM8 |
| Dabigatran | anticoagulant | F2 |
| decitabine | antineoplastic agent | DNMT1 |
| dapoxetine | for treatment of premature ejaculation | SLC6A4 |
| darapladib | antiinflammatory agent, DMARD | PLA2G7 |
| darifenacin | for treatment of overactive bladder | CHRM3 |
| darusentan | antihypertensive agent | EDNRA |
| dasatinib | antineoplastic agent | ABL1 |
| dasatinib | antineoplastic agent | ABL2 |
| dasatinib | antineoplastic agent | EPHA2 |
| dasatinib | antineoplastic agent | FYN |
| dasatinib | antineoplastic agent | KIT |
| dasatinib | antineoplastic agent | LCK |
| dasatinib | antineoplastic agent | PDGFRB |
| dasatinib | antineoplastic agent | SRC |
| dasatinib | antineoplastic agent | STAT5B |
| dasatinib | antineoplastic agent | YES1 |
| methylphenidate | for treatment of ADHD | SLC6A3 |
| DB-959 | antidiabetic | PPARD |
| DB-959 | antidiabetic | PPARG |
| diazoxide choline | antidyslipidaemic agent | ABCC8 |
| DDP225 | for treatment of irritable bowel syndrome | HTR3A |
| DDP225 | for treatment of irritable bowel syndrome | HTR3B |
| DDP225 | for treatment of irritable bowel syndrome | HTR3C |
| DDP225 | for treatment of irritable bowel syndrome | HTR3D |
| DDP225 | for treatment of irritable bowel syndrome | HTR3E |
| DDP225 | for treatment of irritable bowel syndrome | SLC6A2 |
| Debio 0932 | antineoplastic agent | HSP90AA1 |
| Dcbio 0932 | antincoplastic agent | HSP90AB1 |
| DEBIO-9902 SR | for treatment of Alzheimer's disease | ACHE |
| Degarelix | antineoplastic agent | GNRHR |
| Degarelix | antineoplastic agent | GNRHR2 |
| denufosol | for treatment of cystic fibrosis | P2RY2 |
| deoxynojirimycin | for treatment of Pompe disease | GAA |
| bupivacaine | local anestethic, analgesic, neuralgia | SCN10A |
| gabapentin | for treatment of neuropathic pain | CACNA1B |
| gabapentin | for treatment of neuropathic pain | CACNA2D1 |
| gabapentin | for treatment of neuropathic pain | CACNA2D2 |
| romidepsin | antineoplastic agent | HDAC1 |
| romidepsin | antineoplastic agent | HDAC10 |
| romidepsin | antineoplastic agent | HDAC11 |
| romidepsin | antineoplastic agent | HDAC2 |
| romidepsin | antineoplastic agent | HDAC3 |
| romidepsin | antineoplastic agent | HDAC4 |
| romidepsin | antineoplastic agent | HDAC5 |
| romidepsin | antineoplastic agent | HDAC6 |
| romidepsin | antineoplastic agent | HDAC7A |
| romidepsin | antineoplastic agent | HDAC8 |
| romidepsin | antineoplastic agent | HDAC9 |
| dersalazine | antiinflammatory agent, for treatment of | PTGS1 |
| | ulcerative colitis | |
| dersalazine | antiinflammatory agent, for treatment of | PTGS2 |
| | ulcerative colitis | |
| dersalazine | antiinflammatory agent, for treatment of | TNF |
| | ulcerative colitis | |
| desloratadine | antiallergy agent | HRH1 |
| desonide | antiinflammatory agent, corticosteroid | NR3C1 |
| dexamethasone | antiinflammatory agent, glucocorticoid, for | NR3C1 |
| | treatment of Meniere's disease | |
| Dexanabinol | neuroprotectant | GRIN1 |
| Dexanabinol | neuroprotectant | GRIN2A |
| Dexanabinol | neuroprotectant | GRIN2B |
| Dexanabinol | neuroprotectant | GRIN2D |
| Dexanabinol | neuroprotectant | GRIN3A |
| Dexanabinol | neuroprotectant | GRIN3B |
| dexlipotam | for treatment of diabetic neuropathy | PDHB |
| dexloxiglumide | motilitant | CCKAR |
| dexpramipexole | for treatment of amyotrophic lateral sclerosis | DRD2 |
| | (ALS) | |
| dexpramipexole | for treatment of amyotrophic lateral sclerosis | DRD3 |
| | (ALS) | |
| dexpramipexole | for treatment of amyotrophic lateral sclerosis | DRD4 |
| | (ALS) | |
| DG031 | antiinflammatory agent, myocardial infarction | ALOX5AP |
| | prophylaxis | |
| DG041 | Platelet Aggregation Inhibitor | PTGER3 |
| DG051 | antiinflammatory agent, myocardial infarction | LTA4H |
| | prophylaxis | |
| DG071 | for treatment of alzheimer's disease | PDE4A |
| DG071 | for treatment of alzheimer's disease | PDE4B |
| DG3173 | hormone replacement | SSTR1 |
| DG3173 | hormone replacement | SSTR2 |
| DG3173 | hormone replacement | SSTR4 |
| DG3173 | hormone replacement | SSTR5 |
| diazepam | anticonvulsant | GABRA1 |
| diazepam | anticonvulsant | GABRA2 |
| diazepam | anticonvulsant | GABRA3 |
| diazepam | anticonvulsant | GABRA5 |
| diazepam | anticonvulsant | GABRB1 |
| diazepam | anticonvulsant | GABRB2 |
| diazepam | anticonvulsant | GABRB3 |
| diazepam | anticonvulsant | GABRD |
| diazepam | anticonvulsant | GABRE |
| diazepam | anticonvulsant | GABRG1 |
| diazepam | anticonvulsant | GABRG2 |
| diazepam | anticonvulsant | GABRG3 |
| diazepam | anticonvulsant | GABRP |
| diazepam | anticonvulsant | GABRQ |
| diazepam | anticonvulsant | GABRR1 |
| diazepam | anticonvulsant | GABRR2 |
| diazepam | anticonvulsant | GABRR3 |
| diclofenac | analgesic | PTGS1 |
| diclofenac | analgesic | PTGS2 |
| Diclofenac | analgesic | PTGS1 |
| Diclofenac | analgesic | PTGS2 |
| Diclofenac | analgesic | PTGS1 |
| Diclofenac | analgesic | PTGS2 |
| Diclofenac | NSAID | PTGS1 |
| Diclofenac | NSAID | PTGS2 |
| Diclofenac | for treatment of glaucoma | PTGS1 |
| Diclofenac | for treatment of glaucoma | PTGS2 |
| difluprednate | antiinflammatory agent, corticosteroid | NR3C1 |
| diltiazem | antihypertensive agent | CACNG1 |
| latrepirdine | neuroprotectant | ACHE |
| latrepirdine | neuroprotectant | GRIN1 |
| latrepirdine | neuroprotectant | GRIN2A |
| latrepirdine | neuroprotectant | GRIN2B |
| latrepirdine | neuroprotectant | GRIN2C |
| latrepirdine | neuroprotectant | GRIN2D |
| latrepirdine | neuroprotectant | GRIN3A |
| latrepirdine | neuroprotectant | GRIN3B |
| dimiracetam | nootropic | GRIN1 |
| dimiracetam | nootropic | GRIN2A |
| dimiracetam | nootropic | GRIN2B |
| dimiracetam | nootropic | GRIN2C |
| dimiracetam | nootropic | GRIN2D |
| DIO-902 | antidiabetic | ERG11 |
| diquafosol | opthalmological agent | P2RY2 |
| carbidopa | antiparkinson agent | DDC |
| levodopa | antiparkinson agent | DRD1 |
| levodopa | antiparkinson agent | DRD2 |
| omeprazole | antiulcer agent | ATP4A |
| betanechol | antidiabetic | CHRM2 |
| calcitriol | antineoplastic agent | VDR |
| Docetaxel | antineoplastic agent | BCL2 |
| Docetaxel | antineoplastic agent | TBB1 |
| dolasetron | antiemetic | HTR3A |
| dolasetron | antiemetic | HTR3B |
| dolasctron | anticmetic | HTR3C |
| dolasetron | antiemetic | HTR3D |
| dolasetron | antiemetic | HTR3E |
| donepezil | for treatment of alzheimer's disease | ACHE |
| beclomethasone dipropionate | antiinflammatory agent, glucocorticoid | NR3C1 |
| DOV 102,677 | antidepressant | SLC6A2 |
| DOV 102,677 | antidepressant | SLC6A3 |
| DOV 102,677 | antidepressant | SLC6A4 |
| DOV 216,303 | antidepressant | SLC6A2 |
| DOV 216,303 | antidepressant | SLC6A3 |
| DOV 216,303 | antidepressant | SLC6A4 |
| DOV 21947 | antidepressant | SLC6A2 |
| DOV 21947 | antidepressant | SLC6A3 |
| DOV 21947 | antidepressant | SLC6A4 |
| dovitinib | antineoplastic agent | FGFR1 |
| dovitinib | antineoplastic agent | FGFR2 |
| dovitinib | antineoplastic agent | FGFR3 |
| dovitinib | antineoplastic agent | FLT1 |
| dovitinib | antineoplastic agent | FLT1 |
| dovitinib | antineoplastic agent | FLT1 |
| dovitinib | antineoplastic agent | FLT4 |
| dovitinib | antineoplastic agent | KDR |
| dovitinib | antineoplastic agent | PDGFRB |
| doxepin | antimigraine agent | SLC6A2 |
| doxepin | antimigraine agent | SLC6A4 |
| doxercalciferol | for treatment of secondary hyperparathyroidism | VDR |
| doxorubicin | antineoplastic agent | TOP2A |
| doxorubicin | antineoplastic agent | TOP2A |
| doxorubicin | antineoplastic agent | TOP2A |
| doxorubicin | antineoplastic agent | TOP2A |
| DP-VPA | anticonvulsant | ABAT |
| DRF 10945 | antidyslipidaemic agent | PPARA |
| dronabinol | appetite stimulant | CNR1 |
| drospirenone | hormone replacement | PGR |
| estradiol | hormone replacement | ESR1 |
| estradiol | hormone replacement | ESR2 |
| DSC-103 | antiosteoporotic agent | VDR |
| DTS-201 | antineoplastic agent | TOP2A |
| bupivacaine | local anestethic, analgesic, neuralgia | SCN10A |
| bupivacaine | local anestethic, analgesic, neuralgia | SCN10A |
| sildenafil | for treatment of erectile dysfunction | PDE5A |
| dutasteride | for treatment of benign prostate hyperplasia | SRD5A1 |
| dutasteride | for treatment of benign prostate hyperplasia | SRD5A2 |
| tamsulosin | for treatment of benign prostatic hyperplasia | ADRA1A |
| dutasteride | for treatment of benign prostate hyperplasia | SRD5A1 |
| dutogliptin | antidiabetic | DPP4 |
| azelastine | antiallergy agent | HRH1 |
| fluticasone | antiinflammatory agent, glucocorticoid | NR3C1 |
| perampanel | anticonvulsant | GRIA1 |
| perampanel | anticonvulsant | GRIA2 |
| perampanel | anticonvulsant | GRIA3 |
| perampanel | anticonvulsant | GRIA4 |
| E2012 | for treatment of Alzheimer's disease | PSEN1 |
| lenvatinib | antineoplastic agent | FGFR1 |
| lenvatinib | antineoplastic agent | FLT1 |
| lenvatinib | antineoplastic agent | FLT4 |
| lenvatinib | antineoplastic agent | KDR |
| lenvatinib | antineoplastic agent | KIT |
| lenvatinib | antineoplastic agent | PDGFRA |
| lenvatinib | antineoplastic agent | PDGFRB |
| ecabet | antiulcer agent | PGA3 |
| ecabet | antiulcer agent | PGC |
| ecopipam | for treatment of tourettes syndrome, for treatment | DRD1 |
| | of pathological gambling | |
| edoxaban | antithrombotic | F10 |
| venlafaxine | antidepressant | SLC6A2 |
| venlafaxine | antidepressant | SLC6A4 |
| eflornithine | for treatment of unwanted facial hair in women | ODC1 |
| dexamethasone | antiinflammatory agent, glucocorticoid, for | NR3C1 |
| | treatment of Meniere's disease | |
| Etazolate | for treatment of alzheimer's disease | GABRA2 |
| Etazolatc | for treatment of alzheimer's discasc | GABRA3 |
| Etazolate | for treatment of alzheimer's disease | GABRB1 |
| Etazolate | for treatment of alzheimer's disease | GABRB2 |
| Etazolate | for treatment of alzheimer's disease | GABRE |
| Etazolate | for treatment of alzheimer's disease | GABRG1 |
| Etazolate | for treatment of alzheimer's disease | PDE4A |
| Etazolate | for treatment of alzheimer's disease | PDE4B |
| Etazolate | for treatment of alzheimer's disease | PDE4C |
| Etazolate | for treatment of alzheimer's disease | PDE4D |
| ronomilast | antiinflammatory agent | PDE4A |
| ronomilast | antiinflammatory agent | PDE4B |
| ED-71 | antiosteoporotic agent | VDR |
| oxycodone | analgesic | OPRD1 |
| oxycodone | analgesic | OPRK1 |
| oxycodone | analgesic | OPRM1 |
| eliglustat | for treatment of Gaucher's disease | UGCG |
| elinogrel | antiplatelet agent | P2RY12 |
| Elocalcitol | for treatment of benign prostatic hyperplasia | VDR |
| bupropion | antidepressant, appetite suppressant, smoking- | SLC6A2 |
| | cessation agent | |
| bupropion | antidepressant, appetite suppressant, smoking- | SLC6A3 |
| | cessation agent | |
| zonisamide | appetite suppressant | CACNA1G |
| zonisamide | appetite suppressant | CACNA1H |
| zonisamide | appetite suppressant | CACNA1I |
| zonisamide | appetite suppressant | SCN11A |
| zonisamide | appetite suppressant | SCN1A |
| zonisamide | appetite suppressant | SCN1B |
| zonisamide | appetite suppressant | SCN2A |
| zonisamide | appetite suppressant | SCN2B |
| zonisamide | appetite suppressant | SCN3A |
| zonisamide | appetite suppressant | SCN3B |
| zonisamide | appetite suppressant | SCN4A |
| zonisamide | appetite suppressant | SCN4B |
| zonisamide | appetite suppressant | SCN5A |
| zonisamide | appetite suppressant | SCN9A |
| enalapril | antihypertensive agent | ACE |
| fclodipinc | antihypertensive agent | CACNA1C |
| felodipine | antihypertensive agent | CACNA1D |
| felodipine | antihypertensive agent | CACNA1S |
| felodipine | antihypertensive agent | CACNA2D1 |
| felodipine | antihypertensive agent | CACNANB2 |
| paclitaxel | antineoplastic agent | BCL2 |
| paclitaxel | antineoplastic agent | TUBB1 |
| eniluracil | antineoplastic agent | DPYD |
| ENMD-1198 | antineoplastic agent | HIF1A |
| ENMD-2076 | antineoplastic agent | ABL1 |
| ENMD-2076 | antineoplastic agent | AURKA |
| ENMD-2076 | antineoplastic agent | BLK |
| ENMD-2076 | antineoplastic agent | CSF1R |
| ENMD-2076 | antineoplastic agent | FGFR1 |
| ENMD-2076 | antineoplastic agent | FGFR2 |
| ENMD-2076 | antineoplastic agent | FLT3 |
| ENMD-2076 | antineoplastic agent | FLT4 |
| ENMD-2076 | antineoplastic agent | FYN |
| ENMD-2076 | antineoplastic agent | JAK2 |
| ENMD-2076 | antineoplastic agent | KDR |
| ENMD-2076 | antineoplastic agent | KIT |
| ENMD-2076 | antineoplastic agent | LCK |
| ENMD-2076 | antineoplastic agent | NTRK1 |
| ENMD-2076 | antineoplastic agent | PDGFRA |
| ENMD-2076 | antineoplastic agent | PTK2 |
| ENMD-2076 | antineoplastic agent | RET |
| ENMD-2076 | antineoplastic agent | SRC |
| ENMD-2076 | antineoplastic agent | YES1 |
| entacapone | antiparkinson agent | COMT |
| carbidopa | antiparkinson agent | DDC |
| entacapone | antiparkinson agent | COMT |
| levodopa | antiparkinson agent | DRD1 |
| levodopa | antiparkinson agent | DRD2 |
| levodopa | antiparkinson agent | DRD3 |
| levodopa | antiparkinson agent | DRD4 |
| levodopa | antiparkinson agent | DRD5 |
| entinostat | antineoplastic agent | HDAC1 |
| entinostat | antineoplastic agent | HDAC3 |
| Enzastaurin | antincoplastic agent | PRKCB |
| EP217609 | anticoagulant | F10 |
| EP217609 | anticoagulant | F2 |
| EP42675 | anticoagulant | F10 |
| EP42675 | anticoagulant | F2 |
| EPI-743 | for treatment of Chron's disease, for treatment of | NQO1 |
| | ulcerative colitis | |
| epinastine | antiallergy agent | HRH1 |
| epinastine | antiallergy agent | HRH2 |
| eplerenone | antihypertensive agent | NR3C2 |
| eplivanserine | for treatment of insomnia | HTR2A |
| eplivanserine | for treatment of insomnia | HTR2C |
| Epothilone D | antineoplastic agent | TUBB1 |
| eprotirome | antidyslipidaemic agent | THRB |
| erdosteine | for treatment of chronic obstructive pulmonary | ELANE |
| | disorder (COPD) | |
| eritoran | for treatment of sepsis | TLR4 |
| Eslicarbazepine | anticonvulsant | SCN5A |
| esmirtazapine | for treatment of insomnia, for treatment of | ADRA2A |
| | menopausal symptoms | |
| esmirtazapine | for treatment of insomnia, for treatment of | HTR2A |
| | menopausal symptoms | |
| esmirtazapine | for treatment of insomnia, for treatment of | HTR3A |
| | menopausal symptoms | |
| esomeprazole | Proton pump inhibitor | ATP4A |
| estradiol | contraceptive | ESR1 |
| estradiol | contraceptive | ESR1 |
| estradiol | contraceptive | ESR2 |
| norethisterone | contraceptive | PGR |
| estradiol | for treatment of menopausal symptoms | ESR1 |
| estradiol | for treatment of menopausal symptoms | ESR2 |
| estradiol | for treatment of menopausal symptoms | ESR1 |
| estradiol | for treatment of menopausal symptoms | ESR2 |
| estradiol | contraceptive | ESR1 |
| dienogest | contraceptive | ESR1 |
| dienogest | contraceptive | PGR |
| estradiol | contraceptive | ESR2 |
| estradiol | contraceptive | ESR2 |
| estradiol | for treatment of menopausal symptoms | ESR1 |
| estradiol | for treatment of menopausal symptoms | ESR2 |
| levonorgestrel | for treatment of menopausal symptoms | ESR1 |
| levonorgestrel | for treatment of menopausal symptoms | PGR |
| levonorgestrel | for treatment of menopausal symptoms | SRD5A1 |
| estradiol | for treatment of menopausal symptoms | ESR1 |
| estradiol | for treatment of menopausal symptoms | ESR2 |
| estradiol | for treatment of menopausal symptoms | ESR1 |
| estradiol | for treatment of menopausal symptoms | ESR2 |
| drospirenone | contraceptive | AR |
| drospirenone | contraceptive | NR3C2 |
| drospirenonc | contraceptive | PGR |
| estradiol | contraceptive | ESR1 |
| estradiol | contraceptive | ESR2 |
| ethinyl estradiol | contraceptive | ESR1 |
| levonorgestrel | contraceptive | ESR1 |
| levonorgestrel | contraceptive | PGR |
| etilevodopa | antiparkinson agent | DRD1 |
| etilevodopa | antiparkinson agent | DRD2 |
| etilevodopa | antiparkinson agent | DRD3 |
| etilevodopa | antiparkinson agent | DRD4 |
| etilevodopa | antiparkinson agent | DRD5 |
| etodolac | NSAID | PTGS2 |
| etonogestrel | contraceptive | ESR1 |
| etonogestrel | contraceptive | PGR |
| ethinyl estradiol | contraceptive | ESR1 |
| etonogestrel | contraceptive | ESR1 |
| etonogestrel | contraceptive | PGR |
| etoricoxib | NSAID | PTGS2 |
| EV-077-3201-2TBS | antidiabetic | PPARG |
| everolimus | immunosuppressant | MTOR |
| raloxifen | for treatment of menopausal symptoms | ESR1 |
| raloxifen | for treatment of menopausal symptoms | ESR2 |
| metoclopramide | for treatment of diabetic gastroparesis | CHRM1 |
| metoclopramide | for treatment of diabetic gastroparesis | DRD2 |
| EVP-6124 | nootropic | CHRNA7 |
| EVT-101 | antidepressant | GRIN2B |
| EVT-103 | antidepressant | GRIN2B |
| EVT-201 | hypnotic | GABRA2 |
| EVT-201 | hypnotic | GABRA3 |
| EVT-201 | hypnotic | GABRA5 |
| EVT-201 | hypnotic | GABRA6 |
| EVT-201 | hypnotic | GABRB1 |
| EVT-201 | hypnotic | GABRB1 |
| EVT-201 | hypnotic | GABRB2 |
| EVT-201 | hypnotic | GABRB2 |
| EVT-201 | hypnotic | GABRB3 |
| EVT-201 | hypnotic | GABRD |
| EVT-201 | hypnotic | GABRD |
| EVT-201 | hypnotic | GABRE |
| EVT-201 | hypnotic | GABRG1 |
| EVT-201 | hypnotic | GABRG2 |
| EVT-201 | hypnotic | GABRG3 |
| EVT-201 | hypnotic | GABRG3 |
| EVT-201 | hypnotic | GABRP |
| EVT-201 | hypnotic | GABRQ |
| EVT-201 | hypnotic | GABRR2 |
| EVT-302 | smoking-cessation agent | MA0B |
| EVT-401 | antiinflammatory agent | P2RX7 |
| Exebryl-1 | for treatment of alzheimer's disease | APP |
| Exebryl-1 | for treatment of alzheimer's disease | MAPT |
| exemestane | antineoplastic agent | CYP19A1 |
| ezatiostat | for treatment of Myelodysplastic Syndrome | GSTP1 |
| PEG-SN38 | antineoplastic agent | TOP1MT |
| PEG-SN38 | antineoplastic agent | TOP1 |
| fentanyl | analgesic | OPRD1 |
| fentanyl | analgesic | OPRM1 |
| febuxostat | for treatment of gout | XDH |
| felodipine | antihypertensive agent | CACNA1C |
| felodipine | antihypertensive agent | CACNA1D |
| felodipine | antihypertensive agent | CACNA1S |
| felodipine | antihypertensive agent | CACNA2D1 |
| fclodipinc | antihypertensive agent | CACNB2 |
| fenoldopam | antihypertensive agent | DRD1 |
| fenoldopam | antihypertensive agent | DRD5 |
| fenretinide | antineoplastic agent | RARA |
| fenretinide | antineoplastic agent | RARB |
| fenretinide | antineoplastic agent | RARG |
| fentanyl | analgesic | OPRD1 |
| fentanyl | analgesic | OPRM1 |
| fentanyl | analgesic | OPRD1 |
| fentanyl | analgesic | OPRM1 |
| fentanyl | analgesic | OPRD1 |
| fentanyl | analgesic | OPRM1 |
| fentanyl | analgesic | OPRD1 |
| fentanyl | analgesic | OPRM1 |
| fentanyl | analgesic | OPRD1 |
| fentanyl | analgesic | OPRM1 |
| fentanyl | analgesic | OPRD1 |
| fentanyl | analgesic | OPRM1 |
| fentanyl | analgesic | OPRD1 |
| fentanyl | analgesic | OPRM1 |
| fentanyl | analgesic | OPRD1 |
| fentanyl | analgesic | OPRM1 |
| fesoterodine | for treatment of overactive bladder syndrome | CHRM3 |
| fexofenadine | antiallergy agent | HRH1 |
| pseudoephedrine | antiallergy agent | ADRA1A |
| pseudoephedrine | antiallergy agent | ADRA2A |
| pseudoephedrine | antiallergy agent | SLC6A2 |
| pseudoephedrine | antiallergy agent | SLC6A3 |
| pseudoephedrine | antiallergy agent | SLC6A4 |
| FG-2216 | for treatment of anemia | EGLN1 |
| FG-2216 | for treatment of anemia | EGLN2 |
| FG-2216 | for treatment of anemia | EGLN3 |
| FG-4592 | for treatment of anemia | EGLN1 |
| FG-4592 | for treatment of anemia | EGLN2 |
| FG-4592 | for treatment of anemia | EGLN3 |
| fingolimod | for treatment of multiple sclerosis | S1PR1 |
| fipamczolc | antiparkinson agent | ADRA2A |
| fipamezole | antiparkinson agent | ADRA2B |
| fipamezole | antiparkinson agent | ADRA2C |
| icatibant | for treatment of hereditary angioedema | BDKRB2 |
| fispemifene | hormone replacement | ESR1 |
| fispemifene | hormone replacement | ESR2 |
| FK352B | antihypertensive agent | ADORA1 |
| alvocidib | antineoplastic agent | CDC2 |
| alvocidib | antineoplastic agent | CDK10 |
| alvocidib | antineoplastic agent | CDK2 |
| alvocidib | antineoplastic agent | CDK3 |
| alvocidib | antineoplastic agent | CDK4 |
| alvocidib | antineoplastic agent | CDK5 |
| alvocidib | antineoplastic agent | CDK6 |
| alvocidib | antineoplastic agent | CDK7 |
| alvocidib | antineoplastic agent | CDK8 |
| alvocidib | antineoplastic agent | CDK9 |
| flibanserin | for treatment of female sexual dysfunction | HTR1A |
| flibanserin | for treatment of female sexual dysfunction | HTR2A |
| flovagatran | anticoagulant | F2 |
| fludarabine | antineoplastic agent | DCK |
| fludarabine | antineoplastic agent | POLA1 |
| fludarabine | antineoplastic agent | RRM1 |
| flunisolide | antiinflammatory agent, glucocorticoid | NR3C1 |
| flunisolide | antiinflammatory agent, glucocorticoid | NR3C1 |
| fluocinonide | antiinflammatory agent, glucocorticoid | NR3C1 |
| fluoxetine | antidepressant | SLC6A4 |
| flupirtine | analgesic | KCNJ3 |
| flupirtine | analgesic | KCNJ5 |
| flupirtine | analgesic | KCNJ6 |
| flupirtine | analgesic | KCNJ9 |
| fluticasone | antiinflammatory agent, glucocorticoid | NR3C1 |
| fluvastatin | antihypecholesterolemic agent | HMGCR |
| fluvoxamine | antidepressant | SLC6A4 |
| dexmethylphenidate | for treatment of ADHD | SLC6A3 |
| dexmethylphenidate | for treatment of ADHD | SLCA2 |
| forodcsinc | antincoplastic agent | PNP |
| formoterol | bronchodilator | ADRB2 |
| formoterol | for treatment of chronic obstructive pulmonary | ADRB2 |
| | disorder (COPD) | |
| fosphenytoin | anticonvulsant | SCN5A |
| fospropofol | hypnotic and sedative | GABRB2 |
| fospropofol | hypnotic and sedative | GABRB3 |
| fostamatinib | antiinflammatory agent, DMARD | SYK |
| cyclosporine | immunosuppressant | CAMLG |
| cyclosporine | immunosuppressant | PPP3R2 |
| prednisolone | antiinflammatory agent, corticosteroid | NR3C1 |
| frovatriptan | antimigraine agent | HTR1B |
| frovatriptan | antimigraine agent | HTR1D |
| fruquintinib | antineoplastic agent | FLT1 |
| fruquintinib | antineoplastic agent | FLT4 |
| fruquintinib | antineoplastic agent | KDR |
| dexamethasone | antiinflammatory agent, glucocorticoid, for | NR3C1 |
| | treatment of Meniere's disease | |
| fulvestrant | antineoplastic agent | ESR1 |
| leucovorin | adjuvant to chemotherapy | TYMS |
| FX125L | antiasthmatic agent | CCR1 |
| FX125L | antiasthmatic agent | CXCR1 |
| FX125L | antiasthmatic agent | CXCR2 |
| FX125L | antiasthmatic agent | CXCR4 |
| gabapentin | analgesic | CACNA1B |
| gabapentin | analgesic | CACNA2D1 |
| gabapentin | analgesic | CACNA2D2 |
| gaboxadol | hypnotic | GABRA2 |
| gaboxadol | hypnotic | GABRA3 |
| gaboxadol | hypnotic | GABRA5 |
| gaboxadol | hypnotic | GABRA6 |
| gaboxadol | hypnotic | GABRB1 |
| gaboxadol | hypnotic | GABRB1 |
| gaboxadol | hypnotic | GABRB2 |
| gaboxadol | hypnotic | GABRB2 |
| gaboxadol | hypnotic | GABRB3 |
| gaboxadol | hypnotic | GABRD |
| gaboxadol | hypnotic | GABRE |
| gaboxadol | hypnotic | GABRG1 |
| gaboxadol | hypnotic | GABRP |
| galantamine | for treatment of alzheimer's disease | ACHE |
| ganaxolone | anticonvulsant | GABRA1 |
| ganaxolone | anticonvulsant | GABRA2 |
| ganaxolone | anticonvulsant | GABRA3 |
| ganaxolone | anticonvulsant | GABRA4 |
| ganaxolone | anticonvulsant | GABRA5 |
| ganaxolone | anticonvulsant | GABRA6 |
| gantacurium | muscle relaxant, neuromuscular blocking agent | CHRNA2 |
| GDC-0068 | antineoplastic agent | AKT1 |
| GDC-0068 | antineoplastic agent | AKT2 |
| GDC-0068 | antineoplastic agent | AKT3 |
| GDC-0973 | antineoplastic agent | MAP2K1 |
| gemcitabine | antineoplastic agent | RRM1 |
| gepirone | antidepressant | HTR1A |
| progesterone | for prevention of preterm delivery | PGR |
| GGTI-2418 | antineoplastic agent | FNTA |
| GGTI-2418 | antineoplastic agent | PGGT1B |
| GL1001 | for treatment of Chron's disease, for treatment of | ACE2 |
| | ulcerative colitis | |
| glimepiride | antidiabetic | KCNJ1 |
| glimepiride | antidiabetic | ABCC8 |
| glimepiride | antidiabetic | KCNJ11 |
| GLPG0187 | antineoplastic agent | ITGA5 |
| GLPG0187 | antineoplastic agent | ITGAV |
| GLPG0187 | antineoplastic agent | ITGB1 |
| GLPG0187 | antineoplastic agent | ITGB3 |
| GLPG0187 | antineoplastic agent | ITGB5 |
| GLPG0187 | antineoplastic agent | ITGB6 |
| GLPG0259 | antiinflammatory agent, DMARD | MAPKAPK5 |
| GLPG0492 | for treatment of cachexia | AR |
| GLPG0634 | antiinflammatory agent, DMARD | JAK1 |
| GLPG0634 | antiinflammatory agent, DMARD | JAK2 |
| Glufosfamide | antineoplastic agent | SLC2A1 |
| Glufosfamide | antineoplastic agent | SLC2A2 |
| Glufosfamide | antineoplastic agent | SLC2A3 |
| Glufosfamide | antineoplastic agent | SLC2A4 |
| Glufosfamide | antineoplastic agent | SLC2A5 |
| Glufosfamide | antineoplastic agent | SLC5A1 |
| Glufosfamide | antineoplastic agent | SLC5A2 |
| Glufosfamide | antineoplastic agent | SLC5A4 |
| glyburide | antidiabetic | ABCC8 |
| metformin | antidiabetic | PRKAB1 |
| glycopyrrolate | antineoplastic agent | CHRM1 |
| GMI-1070 | for treatment of sickle-cell disease | SELE |
| GMI-1070 | for treatment of sickle-cell disease | SELL |
| GMI-1070 | for treatment of sickle-cell disease | SELP |
| GMX1777 | antineoplastic agent | NAMPT |
| NBI-42902 | for treatment of postmenopausal | GNRHR |
| | symptoms, antineoplastic agent | |
| NBI-42902 | for treatment of postmenopausal | GNRHR2 |
| | symptoms, antineoplastic agent | |
| GPI-1485 | antiparkinson agent | FKBP1A |
| GPX-100 | antineoplastic agent | TOP2A |
| granisetron | antiemetic | HTR3A |
| granisetron | antiemetic | HTR3B |
| granisetron | antiemetic | HTR3C |
| granisetron | antiemetic | HTR3D |
| granisetron | antiemetic | HTR3E |
| granisetron | antiemetic | HTR3A |
| granisetron | antiemetic | HTR3B |
| granisetron | antiemetic | HTR3C |
| granisetron | antiemetic | HTR3D |
| granisetron | antiemetic | HTR3E |
| GS-9411 | for treatment of pulmonary disease | SCNN1A |
| GS-9411 | for treatment of pulmonary disease | SCNN1B |
| GS-9411 | for treatment of pulmonary disease | SCNN1D |
| GS-9411 | for treatment of pulmonary disease | SCNN1G |
| GSI-136 | for treatment of Alzheimer's discasc | APH1A |
| GSI-136 | for treatment of Alzheimer's disease | APH1B |
| GSI-136 | for treatment of Alzheimer's disease | NCSTN |
| GSI-136 | for treatment of Alzheimer's disease | PSEN1 |
| GSI-136 | for treatment of Alzheimer's disease | PSEN2 |
| GSI-136 | for treatment of Alzheimer's disease | PSENEN |
| GSK-1004723 | antiallergy agent | HRH1 |
| GSK-1004723 | antiallergy agent | HRH3 |
| trametinib | antineoplastic agent | MAP2K1 |
| GSK2118436 | antineoplastic agent | BRAF |
| GSK-961081 | bronchodilator | ADRB2 |
| GSK-961081 | bronchodilator | CHRM3 |
| GTS-21 | for treatment of schizophrenia | CHRNA7 |
| GTx-758 | antineoplastic agent | LHCGR |
| guanfacine | for treatment of ADHD | ADRA2A |
| GW501516 | antidyslipidaemic agent | PPARA |
| GW501516 | antidyslipidaemic agent | PPARD |
| GW501516 | antidyslipidaemic agent | PPARG |
| GW642444 | bronchodilator | ADRB2 |
| halofuginone | antineoplastic agent | EPRS |
| flurbiprofen | antiinflammatory agent, NSAID | PTGS2 |
| nitric oxide | antiinflammatory agent | GUCY1A2 |
| HE3235 | antineoplastic agent | AR |
| doxorubicin | antineoplastic agent | TOP2A |
| heparin | anticoagulant | F10 |
| heparin | anticoagulant | SERPINC1 |
| heparin | anticoagulant | F10 |
| heparin | anticoagulant | SERPINC1 |
| HF0220 | for treatment of alzheimer's disease | unknown |
| HGS1029 | antineoplastic agent | BIRC2 |
| HGS1029 | antineoplastic agent | BIRC3 |
| HGS1029 | antineoplastic agent | BIRC5 |
| HGS1029 | antineoplastic agent | XIAP |
| amlodipine | antihypertensive agent | CACNA1C |
| amlodipine | antihypertensive agent | CACNA1D |
| amlodipine | antihypertensive agent | CACNA1S |
| amlodipine | antihypertensive agent | CACNA2D1 |
| amlodipine | antihypertensive agent | CACNAB2 |
| simvastatin | antihypertensive agent | HMGCR |
| amiloride | antihypertensive agent | SCNN1A |
| amiloride | antihypertensive agent | SCNN1B |
| amiloride | antihypertensive agent | SCNN1D |
| amiloride | antihypertensive agent | SCNN1G |
| spironolactone | antihypertensive agent | NR3C2 |
| huperzine-A | for treatment of Alzheimer's disease | ACHE |
| hydralazine | antihypertensive agent | AOC3 |
| isosorbide dinitrate | antihypertensive agent | NPR1 |
| hydroxytamoxifen | for treatment of cyclic mastalgia | ESR1 |
| hydroxytamoxifen | for treatment of cyclic mastalgia | ESR2 |
| famotidine | acid reducer | HRH2 |
| famotidine | for treatment of gastric ulcer and | HRH2 |
| | gastroesophageal reflux | |
| ibuprofen | NSAID | PTGS1 |
| ibuprofen | NSAID | PTGS2 |
| ibandronate | antiosteoporotic agent | FDPS |
| dexamethasone | antiinflammatory agent, glucocorticoid, for | NR3C1 |
| | treatment of Meniere's disease | |
| ibudilast | neuroprotectant | PDE4A |
| ibudilast | neuroprotectant | PDE4B |
| ibudilast | neuroprotectant | PDE4C |
| ICA-105665 | anticonvulsant | KCNQ1 |
| ICA-105665 | anticonvulsant | KCNQ2 |
| ICA-105665 | anticonvulsant | KCNQ3 |
| ICA-105665 | anticonvulsant | KCNQ4 |
| ICA-105665 | anticonvulsant | KCNQ5 |
| idrabiotaparinux | antithrombotic | F10 |
| idraparinux | antithrombotic | F10 |
| iferanserin | antihemorrhoidal agent | HTR2A |
| iloperidone | antipsychotic agent, atypical | ADRA1A |
| iloperidone | antipsychotic agent, atypical | ADRA2C |
| iloperidone | antipsychotic agent, atypical | DRD1 |
| iloperidone | antipsychotic agent, atypical | DRD2 |
| iloperidone | antipsychotic agent, atypical | DRD3 |
| iloperidone | antipsychotic agent, atypical | HRH1 |
| iloperidone | antipsychotic agent, atypical | HTR1A |
| iloperidone | antipsychotic agent, atypical | HTR2A |
| iloperidone | antipsychotic agent, atypical | HTR6 |
| iloperidone | antipsychotic agent, atypical | HTR7 |
| iloprost | antihypertensive agent | PTGER1 |
| iloprost | antihypertensive agent | PTGIR |
| fluocinolone acetonide | antiinflammatory agent, glucocorticoid | NR3C1 |
| imatinib | antineoplastic agent | ABL1 |
| imatinib | antineoplastic agent | CSF1R |
| imatinib | antineoplastic agent | DDR1 |
| imatinib | antineoplastic agent | KIT |
| imatinib | antineoplastic agent | NTRK1 |
| imatinib | antineoplastic agent | PDGFRA |
| imatinib | antineoplastic agent | PDGFRB |
| imatinib | antineoplastic agent | RET |
| Imiquimod | anti wart agent, antineoplastic agent | TLR7 |
| implitapide | antiatherosclerotic agent | MTTP |
| INCB13739 | antidiabetic | HSD11B1 |
| INCB18424 | antineoplastic agent, antiinflammatory agent | JAK1 |
| INCB18424 | antineoplastic agent, antiinflammatory agent | JAK2 |
| INCB3284 | antiinflammatory agent, DMARD | CCR2 |
| INCB7839 | antineoplastic agent | ADAM10 |
| INCB7839 | antineoplastic agent | ADAM17 |
| indacaterol | bronchodilator | ADRB2 |
| indomethacin | NSAID | KCNE1 |
| indomethacin | NSAID | KCNQ1 |
| Indiplon | hypnotic | GABRA1 |
| inecalcitol | antineoplastic agent, prostate cancer | VDR |
| apomorphine | for treatment of sexual dysfunction in | DRD2 |
| | women, for treatment of erectile | |
| | dysfunction, antiparkinson agent | |
| apomorphine | for treatment of sexual dysfunction in | DRD3 |
| | women, for treatment of erectile | |
| | dysfunction, antiparkinson agent | |
| apomorphine | for treatment of sexual dysfunction in | DRD4 |
| | women, for treatment of erectile | |
| | dysfunction, antiparkinson agent | |
| atropine | nerve agent antidote | CHRM1 |
| atropine | nerve agent antidote | CHRM2 |
| atropine | nerve agent antidote | CHRM3 |
| atropine | nerve agent antidote | CHRM4 |
| atropine | nerve agent antidote | CHRM5 |
| iniparib | antineoplastic agent | PARP1 |
| INK128 | antineoplastic agent | CRTC1 |
| INK128 | antineoplastic agent | CRTC2 |
| INNO-206 | antineoplastic agent | TOP2A |
| INO-8875 | for treatment of glaucoma | ADORA1 |
| INS37217 | for treatment of rhegmatogenous retinal | P2RY2 |
| | detachment | |
| INS37217 | for treatment of cystic fibrosis, for treatment of | P2RY2 |
| | perennial allergic rhinitis | |
| INSM-18 | antineoplastic agent, prostate cancer | ERBB2 |
| INSM-18 | antineoplastic agent, prostate cancer | IGF1R |
| AMG-131 | antidiabetic | PPARG |
| apomorphine | for treatment of sexual dysfunction in | DRD2 |
| | women, for treatment of erectile | |
| | dysfunction, antiparkinson agent | |
| apomorphine | for treatment of sexual dysfunction in | DRD3 |
| | women, for treatment of erectile | |
| | dysfunction, antiparkinson agent | |
| apomorphine | for treatment of sexual dysfunction in | DRD4 |
| | women, for treatment of erectile | |
| | dysfunction, antiparkinson agent | |
| ketorolac | NSAID | PTGS2 |
| morphine | analgesic | OPRD1 |
| morphine | analgesic | OPRK1 |
| morphine | analgesic | OPRM1 |
| retaspimycin | antineoplastic agent | HSP90AA1 |
| retaspimycin | antineoplastic agent | HSP90AA2 |
| retaspimycin | antineoplastic agent | HSP90AB1 |
| IPI-504 | antineoplastic agent | HSP90AA1 |
| IPI-504 | antineoplastic agent | HSP90AA2 |
| IPI-504 | antineoplastic agent | HSP90AB1 |
| IPI-940 | analgesic | FAAH |
| ipratropium | for treatment of chronic obstructive pulmonary | CHRM1 |
| | disorder (COPD) | |
| ipratropium | for treatment of chronic obstructive pulmonary | CHRM2 |
| | disorder (COPD) | |
| salbutamol | for treatment of chronic obstructive pulmonary | ADRB2 |
| | disorder (COPD) | |
| IPX066 | antiparkinson agent | DDC |
| irbesartan | antihypertensive agent | AGTR1 |
| gefitinib | antineoplastic agent | EGFR |
| irinotecan | antincoplastic agent | TOP1 |
| isofagominc | for treatment of Gaucher's discasc | GBA |
| ispinesib | antineoplastic agent | KIF11 |
| istaroxime | for treatment of heart failure | ATP1A1 |
| istaroxime | for treatment of heart failure | ATP2A2 |
| istradefylline | antiparkinson agent | ADORA2A |
| bromfenac | opthalmological agent, NSAID | PTGS1 |
| bromfenac | opthalmological agent, NSAID | PTGS2 |
| bromfenac | opthalmological agent, NSAID | PTGS1 |
| bromfenac | opthalmological agent, NSAID | PTGS2 |
| Givinostat | antineoplastic agent, antiinflammatory agent | HDAC1 |
| Givinostat | antineoplastic agent, antiinflammatory agent | HDAC10 |
| Givinostat | antineoplastic agent, antiinflammatory agent | HDAC2 |
| Givinostat | antineoplastic agent, antiinflammatory agent | HDAC3 |
| Givinostat | antineoplastic agent, antiinflammatory agent | HDAC4 |
| Givinostat | antineoplastic agent, antiinflammatory agent | HDAC5 |
| Givinostat | antineoplastic agent, antiinflammatory agent | HDAC6 |
| Givinostat | antineoplastic agent, antiinflammatory agent | HDAC7 |
| Givinostat | antineoplastic agent, antiinflammatory agent | HDAC8 |
| Givinostat | antineoplastic agent, antiinflammatory agent | HDAC9 |
| ITI-007 | antipsychotic agent | DRD2 |
| ITI-007 | antipsychotic agent | HTR2A |
| ITI-007 | antipsychotic agent | PPP1R1B |
| ITI-007 | antipsychotic agent | SLC6A4 |
| itopride | motilitant | ACHE |
| itopride | motilitant | DRD2 |
| IW-6118 | analgesic | FAAH |
| ixabepilone | antineoplastic agent | TUBB3 |
| JB991 | antiinflammatory agent, dermatologic agent | PPARG |
| JNJ-37822681 | antipsychotic agent | DRD2 |
| JSM 6427 | for treatment of age-related macular | ITGA5 |
| | degeneration | |
| JSM 6427 | for treatment of age-related macular | ITGB1 |
| | degeneration | |
| ropinirole | for treatment of restlegs legs syndrome | DRD2 |
| ropinirole | for treatment of restlegs legs syndrome | DRD3 |
| ropinirole | for treatment of restlegs legs syndrome | DRD4 |
| clonazepam | anticonvulsant | GABRA2 |
| clonazepam | anticonvulsant | GABRA3 |
| clonazepam | anticonvulsant | GABRA5 |
| clonazepam | anticonvulsant | GABRA6 |
| clonazepam | anticonvulsant | GABRB1 |
| clonazepam | anticonvulsant | GABRB1 |
| clonazepam | anticonvulsant | GABRB2 |
| clonazepam | anticonvulsant | GABRB2 |
| clonazepam | anticonvulsant | GABRB3 |
| clonazepam | anticonvulsant | GABRD |
| clonazepam | anticonvulsant | GABRD |
| clonazepam | anticonvulsant | GABRE |
| clonazepam | anticonvulsant | GABRG2 |
| clonazepam | anticonvulsant | GABRG3 |
| clonazepam | anticonvulsant | GABRG3 |
| clonazepam | anticonvulsant | GABRP |
| clonazepam | anticonvulsant | GABRQ |
| clonazepam | anticonvulsant | GABRR2 |
| Karenitecin | antineoplastic agent | TOP1 |
| KC706 | antiinflammatory agent, DMARD | MAPK11 |
| KC706 | antiinflammatory agent, DMARD | MAPK12 |
| KC706 | antiinflammatory agent, DMARD | MAPK13 |
| KC706 | antiinflammatory agent, DMARD | MAPK14 |
| KD3010 | antiobesity agent, for treatment of metabolic | PPARD |
| | disorders | |
| ketoprofen | NSAID | PTGS1 |
| ketoprofen | NSAID | PTGS2 |
| ketoprofen | NSAID | PTGS1 |
| ketoprofen | NSAID | PTGS1 |
| ketoprofen | NSAID | PTGS2 |
| ketoprofen | NSAID | PTGS2 |
| ketorolac | NSAID | PTGS1 |
| ketorolac | NSAID | PTGS2 |
| ketotifen | antiallergy agent | HRH1 |
| ketoprofen | NSAID | PTGS1 |
| ketoprofen | NSAID | PTGS1 |
| ketoprofen | NSAID | PTGS2 |
| ketoprofen | NSAID | PTGS2 |
| KN38-7271 | neuroprotectant | CNR1 |
| KN38-7271 | neuroprotectant | CNR2 |
| KOS-2187 | for treatment of gastrointestinal motility | MLNR |
| | disorders | |
| kp201 | analgesic | OPRD1 |
| kp201 | analgesic | OPRK1 |
| kp201 | analgesic | OPRM1 |
| KRP-104 | antidiabetic | DPP4 |
| KUC-7483 | for treatment of overactive bladder | ADRB3 |
| KX2-391 | antineoplastic agent | SRC |
| granisetron | antiemetic | HTR3A |
| Lacosamide | anticonvulsant, analgesic, neuropathic pain | DPYSL2 |
| lamotrigine | anticonvulsant | SCN2A |
| lanreotide | for treatment of acromegaly | SSTR1 |
| lanreotide | for treatment of acromegaly | SSTR5 |
| lansoprazole | antiulcer agent | ATP4A |
| lansoprazole | antiulcer agent | ATP4A |
| LAS-100977 | bronchodilator | ADRB2 |
| lasmiditan | antimigraine agent | HTR1F |
| lasofoxifene | antiosteoporotic agent, hormone replacement | ESR1 |
| | therapy | |
| latanoprost | for treatment of glaucoma | PTGFR |
| timolol | for treatment of glaucoma | ADRB1 |
| timolol | for treatment of glaucoma | ADRB2 |
| latanoprost | for treatment of glaucoma | PTGFR |
| latanoprost | for treatment of glaucoma | PTGFR |
| atorvastatin | anticholesterolaemic agent | HMGCR |
| fenofibrate | anticholesterolaemic agent | PPARA |
| fenofibrate | anticholesterolaemic agent | PPARA |
| sirolimus | immunosuppressant | FGF2 |
| sirolimus | immunosuppressant | FKBP1A |
| sirolimus | immunosuppressant | FRAP1 |
| Erismodegib | antineoplastic agent | SMO |
| LEE011 | antineoplastic agent | CDK4 |
| LEE011 | antineoplastic agent | CDK6 |
| lercanidipine | antihypertensive agent | CACNG1 |
| LE-SN38 | antineoplastic agent | TOP1 |
| LE-SN38 | antineoplastic agent | TOP1MT |
| lesogaberan | for treatment of gastrointestinal reflux disease | GABBR1 |
| lesogaberan | for treatment of gastrointestinal reflux disease | GABBR2 |
| lestaurtinib | antineoplastic agent | FLT3 |
| lestaurtinib | antineoplastic agent | NTRK1 |
| lestaurtinib | antineoplastic agent | NTRK2 |
| lestaurtinib | antineoplastic agent | NTRK3 |
| lestaurtinib | antineoplastic agent | JAK2 |
| ambrisentan | antihypertensive agent | EDNRA |
| ambrisentan | antihypertensive agent | EDNRB |
| letrozole | antineoplastic agent | CYP19A1 |
| salbutamol | bronchodilator | ADRB2 |
| levetiracetam | anticonvulsant | CACNA1B |
| levetiracetam | anticonvulsant | SV2A |
| levocetirizine | antiallergy agent | HRH1 |
| levodopa | antiparkinson agent | DRD1 |
| levodopa | antiparkinson agent | DRD2 |
| levodopa | antiparkinson agent | DRD3 |
| levodopa | antiparkinson agent | DRD4 |
| levodopa | antiparkinson agent | DRD5 |
| levomilnacipran | antidepressant | SLC6A2 |
| levomilnacipran | antidepressant | SLC6A4 |
| ethinyl estradiol | contraceptive | ESR1 |
| levonorgestrel | contraceptive | ESR1 |
| levonorgestrel | contraceptive | PGR |
| levonorgestrel | contraceptive | SRD5A1 |
| Levosimendan | for treatment of heart failure | KCNJ11 |
| Levosimendan | for treatment of heart failure | TNNC1 |
| levothyroxine | hormone replacement | THRA |
| levothyroxine | hormone replacement | THRB |
| levothyroxine | hormone replacement | THRA |
| levothyroxine | hormone replacement | THRB |
| LGD-1550 | antineoplastic agent | RARA |
| LGD-1550 | antineoplastic agent | RARB |
| LGD-1550 | antineoplastic agent | RARG |
| LGD-2941 | antiosteoporotic agent | AR |
| LGD-4033 | hormone replacement | AR |
| LGD-4665 | thrombopoietic agent | MPL |
| Liarozole | dermatological agent, for treatment of ichtyosis | CYP26A1 |
| licarbazepine | for treatment of bipolar disorder | SCN5A |
| licofelone | antiinflammatory agent | ALOX5 |
| licofelone | antiinflammatory agent | PTGS2 |
| lidocaine | anestethic | SCN9A |
| lidocaine | anestethic | SCN10A |
| lidocaine | anestethic | SCN5A |
| piroxicam | antiinflammatory agent, NSAID | PTGS2 |
| lidocaine | anestethic | SCN10A |
| lidocaine | anestethic | SCN5A |
| lidocaine | anestethic | SCN9A |
| lidocaine | anestethic | SCN10A |
| lidocaine | anestethic | SCN5A |
| lidocaine | anestethic | SCN9A |
| lidocaine | anestethic | SCN10A |
| lidocaine | anestethic | SCN5A |
| lidocaine | anestethic | SCN9A |
| LIM-0705 | for improving pharmacokinetics of tacrolimus | ABCA5 |
| LIM-0705 | for improving pharmacokinetics of tacrolimus | ABCB1 |
| Linaglipton | antidiabetic | DPP4 |
| fluticasone propionate | for treatment of symptomatic exophthalmos | NR3C1 |
| | associated with thyroid-related eye disease | |
| salbutamol | for treatment of symptomatic exophthalmos | ADRB2 |
| | associated with thyroid-related eye disease | |
| docetaxel | antincoplastic agent | BCL2 |
| docetaxel | antineoplastic agent | TUBB1 |
| doxorubicin | antineoplastic agent | TOP2A |
| paclitaxel | antineoplastic agent | TOP2A |
| lurtotecan | antineoplastic agent | TOP1 |
| mitoxantrone | antineoplastic agent | TOP2A |
| prednisolone | antiinflammatory agent, corticosteroid | NR3C1 |
| Lipotecan | antineoplastic agent | TOP1 |
| lisinopril | antihypertensive agent | ACE |
| Lisofylline | antidiabetic | STAT4 |
| lixivaptan | for treatment of hyponatremia | AVPR2 |
| Lobeline | for treatment of metamphetamine addicton | SLC18A2 |
| lofexidine | for treatment of opiate withdrawal | ADRA2A |
| lofexidine | for treatment of opiate withdrawal | ADRA2B |
| lofexidine | for treatment of opiate withdrawal | ADRA2C |
| lomitapide | anticholesterolaemic agent | MTTP |
| LOR-253 | antineoplastic agent | MTF1 |
| loratadine | antiasthmatic agent | HRH1 |
| montelukast | antiasthmatic agent | CYSLTR1 |
| Lorcaserin | antiobesity agent | HTR2C |
| loteprednol etabonate | antiinflammatory agent, corticosteroid | NR3C1 |
| methamphetamine | neuroprotectant | ADRA2A |
| methamphetamine | neuroprotectant | ADRA2B |
| methamphetamine | neuroprotectant | ADRA2C |
| methamphetamine | neuroprotectant | MAOA |
| methamphetamine | neuroprotectant | MAOB |
| methamphetamine | neuroprotectant | SLC18A1 |
| methamphetamine | neuroprotectant | SLC18A2 |
| methamphetamine | neuroprotectant | SLC6A2 |
| methamphetamine | neuroprotectant | SLC6A3 |
| methamphetamine | neuroprotectant | SLC6A4 |
| methamphetamine | neuroprotectant | TAAR1 |
| lovastatin | anticholesterolaemic agent | HMGCR |
| enoxaparin | anticoagulant | F2 |
| vortioxetine | antidepressant | HTR1A |
| vortioxetine | antidepressant | HTR1B |
| vortioxetine | antidepressant | HTR3A |
| vortioxetine | antidepressant | HTR7 |
| vortioxetine | antidepressant | SLC6A4 |
| Tedatioxetine | antidepressant | ADRA1A |
| Tedatioxetine | antidepressant | HTR2C |
| Tedatioxetine | antidepressant | HTR2C |
| Tedatioxetine | antidepressant | HTR3A |
| Tedatioxetine | antidepressant | SLC6A2 |
| Tedatioxetine | antidepressant | SLC6A3 |
| Tedatioxetine | antidepressant | SLC6A4 |
| zicronapine | antipsychotic agent | DRD4 |
| Lu-AE58054 | antipsychotic agent | HTR6 |
| Lubiprostone | motilitant, for treatment of irritable bowel | CLCN2 |
| | disorder | |
| lumiracoxib | NSAID | PTGS2 |
| eszopiclone | hypnotic | GABRA1 |
| eszopiclone | hypnotic | GABRA2 |
| eszopiclone | hypnotic | GABRA3 |
| eszopiclone | hypnotic | GABRA5 |
| eszopiclone | hypnotic | TSPO |
| lurasidone | antipsychotic agent | ADRA2C |
| lurasidone | antipsychotic agent | DRD2 |
| lurasidone | antipsychotic agent | HTR1A |
| lurasidone | antipsychotic agent | HTR2A |
| lurasidone | antipsychotic agent | HTR7 |
| LX1031 | for treatment of irritable bowel syndrome | TPH1 |
| LX1032 | for treatment of carcinoid syndrome | TPH1 |
| cyclosporine A | immunosuppressant, opthalmological agent | CAMLG |
| cyclosporine A | immunosuppressant, opthalmological agent | PPP3R2 |
| LX4211 | antidiabetic | SLC5A1 |
| LX4211 | antidiabetic | SLC5A2 |
| LY2140023 | antipsychotic agent | GRM2 |
| LY2140023 | antipsychotic agent | GRM3 |
| LY3009104 | antiinflammatory agent, DMARD | JAK1 |
| LY3009104 | antiinflammatory agent, DMARD | JAK2 |
| semagacestat | for treatment of Alzheimer's disease | PSEN1 |
| semagacestat | for treatment of Alzheimer's disease | PSEN2 |
| LY-517717 | anticoagulant | F10 |
| navoglitazar | antidiabetic | PPARA |
| naveglitazar | antidiabetic | PPARG |
| LY-674 | anticholesterolaemic agent | PPARA |
| M0002 | for treatemnt of ascites | AVPR2 |
| heparin | anticoagulant | F10 |
| heparin | anticoagulant | HPSE |
| heparin | anticoagulant | SERPINC1 |
| morphine | analgesic | OPRD1 |
| morphine | analgesic | OPRK1 |
| morphine | analgesic | OPRM1 |
| macitentan | cardiovascular agent | EDNRA |
| macitentan | cardiovascular agent | EDNRB |
| dihydroergotamine | antimigraine agent | HTR1B |
| dihydroergotamine | antimigraine agent | HTR1D |
| budesonide | antiinflammatory agent, glucocorticoid | NR3C1 |
| formoterol | bronchodilator | ADRB2 |
| budesonide | antiinflammatory agent, glucocorticoid | NR3C1 |
| masitinib | antiinflammatory agent, DMARD, antineoplastic | ABL1 |
| | agent | |
| masitinib | antiinflammatory agent, DMARD, antineoplastic | CSF1R |
| | agent | |
| masitinib | antiinflammatory agent, DMARD, antineoplastic | HCK |
| | agent | |
| masitinib | antiinflammatory agent, DMARD, antineoplastic | KIT |
| | agent | |
| masitinib | antiinflammatory agent, DMARD, antineoplastic | LYN |
| | agent | |
| masitinib | antiinflammatory agent, DMARD, antineoplastic | PDGFRA |
| | agent | |
| masitinib | antiinflammatory agent, DMARD, antineoplastic | PDGFRB |
| | agent | |
| masitinib | antiinflammatory agent, DMARD, antineoplastic | SRC |
| | agent | |
| mesalazine | for treatment of ulcerative proctitis | ALOX5 |
| mesalazine | for treatment of ulcerative proctitis | PPARG |
| mesalazine | for treatment of ulcerative proctitis | PTGS1 |
| mesalazine | for treatment of ulcerative proctitis | PTGS2 |
| MB07811 | antidyslipidaemic agent | THRB |
| MBX-2044 | antidiabetic | PPARG |
| MBX-2982 | antidiabetic | GPR119 |
| MBX-8025 | antidyslipidaemic agent | PPARD |
| lisinopril | antihypertensive agent | ACE |
| lisinopril | antihypertensive agent | ACE2 |
| MC-1 | cardioprotectant | LPAR4 |
| MC-1 | cardioprotectant | LPAR6 |
| MC-1 | cardioprotectant | P2RY1 |
| MC-1 | cardioprotectant | P2RY10 |
| MC-1 | cardioprotectant | P2RY11 |
| MC-1 | cardioprotectant | P2RY12 |
| MC-1 | cardioprotectant | P2RY13 |
| MC-1 | cardioprotectant | P2RY14 |
| MC-1 | cardioprotectant | P2RY2 |
| MC-1 | cardioprotectant | P2RY4 |
| MC-1 | cardioprotectant | P2RY6 |
| MC-1 | cardioprotectant | P2RY8 |
| MCD-386 | for treatment of Alzheimer's disease | CHRM1 |
| MDAM | antineoplastic agent | DHFR |
| MDV3100 | antineoplastic agent | AR |
| Mebendazole | antineoplastic agent | TUBA1A |
| Mebendazole | antineoplastic agent | TUBB2C |
| mecamylamine | for treatment of ADHD | CHRNA2 |
| melogliptin | antidiabetic | DPP4 |
| MEM 1003 | for treatment of Alzheimer's disease | CACNA1C |
| MEM 1003 | for treatment of Alzheimer's disease | CACNA1D |
| MEM 1003 | for treatment of Alzheimer's disease | CACNA1F |
| MEM 1003 | for treatment of Alzheimer's disease | CACNA1S |
| MEM 1414 | for treatment of Alzheimer's disease | PDE4A |
| MEM 1414 | for treatment of Alzheimer's disease | PDE4B |
| MEM 63908 | for treatment of Alzheimer's disease | CHRNA7 |
| MEM3454 | for treatment of Alzheimer's disease | CHRNA7 |
| memantine | for treatment of glaucoma | GRIN2A |
| memantine | for treatment of glaucoma | GRIN2B |
| memantine | for treatment of glaucoma | GRIN3A |
| vorinostat | antineoplastic agent | HDAC1 |
| vorinostat | antincoplastic agent | HDAC2 |
| vorinostat | antineoplastic agent | HDAC3 |
| vorinostat | antineoplastic agent | HDAC6 |
| mesalamine | antiinflammatory agent | ALOX5 |
| mesalamine | antiinflammatory agent | PPARG |
| mesalamine | antiinflammatory agent | PTGS1 |
| mesalamine | antiinflammatory agent | PTGS2 |
| WX-671 | antineoplastic agent | PLAU |
| Oxypurinol | for treatment of heart failure, for treatment of | XDH |
| | gout | |
| metaglidasen | antidiabetic | PPARG |
| metformin | antidiabetic | PRKAB1 |
| metformin | antidiabetic | PRKAB1 |
| metformin | antidiabetic | PRKAB1 |
| Methylnaltrexone | for treatment of opioid-induced constipation | OPRM1 |
| methylphenidate | for treatment of ADHD | SLC6A2 |
| methylphenidate | for treatment of ADHD | SLC6A3 |
| methylphenidate | for treatment of ADHD | SLC6A4 |
| methylphenidate | for treatment of ADHD | SLC6A2 |
| methylphenidate | for treatment of ADHD | SLC6A3 |
| methylphenidate | for treatment of ADHD | SLC6A4 |
| methylphenidate | for treatment of ADHD | SLC6A2 |
| methylphenidate | for treatment of ADHD | SLC6A3 |
| methylphenidate | for treatment of ADHD | SLC6A4 |
| methyltestosterone | for treatment of dysfunctional libido in women | AR |
| metoclopramide | motilitant, for treatment of gastroesophageal | CHRM1 |
| | reflux disease | |
| metoclopramide | motilitant, for treatment of gastroesophageal | DRD2 |
| | reflux disease | |
| metoclopramide | antiemetic | CHRM1 |
| metoclopramide | antiemetic | DRD2 |
| metoprolol | antihypertensive agent | ADRB1 |
| MF101 | for treatment of menopausal symptoms | ESR2 |
| MGCD-0103 | antineoplastic agent | HDAC1 |
| MGCD-0103 | antineoplastic agent | HDAC10 |
| MGCD-0103 | antincoplastic agent | HDAC11 |
| MGCD-0103 | antineoplastic agent | HDAC2 |
| MGCD-0103 | antineoplastic agent | HDAC3 |
| MGCD-0103 | antineoplastic agent | HDAC4 |
| MGCD-0103 | antineoplastic agent | HDAC5 |
| MGCD-0103 | antineoplastic agent | HDAC6 |
| MGCD-0103 | antineoplastic agent | HDAC7A |
| MGCD-0103 | antineoplastic agent | HDAC8 |
| MGCD-0103 | antineoplastic agent | HDAC9 |
| MGCD265 | antineoplastic agent | FLT1 |
| MGCD265 | antineoplastic agent | FLT4 |
| MGCD265 | antineoplastic agent | KDR |
| MGCD265 | antineoplastic agent | MET |
| MGCD265 | antineoplastic agent | MST1R |
| MGCD265 | antineoplastic agent | TEK |
| morphine | analgesic | OPRD1 |
| morphine | analgesic | OPRK1 |
| morphine | analgesic | OPRM1 |
| paclitaxel | antiinflammatory agent, DMARD | BCL2 |
| paclitaxel | antiinflammatory agent, DMARD | TUBB1 |
| Midostaurin | antineoplastic agent | FLT3 |
| Mifepristone | opthalmological agent, for lowering intraocular | NR3C1 |
| | pressure | |
| Mifepristone | opthalmological agent, for lowering intraocular | PGR |
| | pressure | |
| Mifepristone | antipsychotic, antidepressant | NR3C1 |
| Mifepristone | antipsychotic, antidepressant | PGR |
| migalastat | enzyme replacement therapy, for treatment of | GLA |
| | Fabry disease | |
| miglustat | for treatment of Gaucher's disease | UGCG |
| milataxel | antineoplastic agent | BCL2 |
| milataxel | antineoplastic agent | TUBB1 |
| Milnacipran | for treatment of fibromyalgia syndrome | SLC6A2 |
| Milnacipran | for treatment of fibromyalgia syndrome | SLC6A4 |
| milveterol | bronchodilator | ADRB2 |
| MIM-D3 | opthalmological agent | NTRK1 |
| minodronate | antineoplastic agent | FDPS |
| pramipexole | antiparkinson agent | DRD2 |
| pramipexole | antiparkinson agent | DRD3 |
| pramipexole | antiparkinson agent | DRD4 |
| mirtazapine | antidepressant | ADRA2A |
| mirtazapine | antidepressant | HTR2A |
| mirtazapine | antidepressant | HTR3A |
| mitemcinal | for treatment of gastroparesis | MLNR |
| mitiglinide | antidiabetic | ABCC8 |
| mitoxantrone | antineoplastic agent | TOP2A |
| MIV-701 | for treatment of osteoporosis | CTSK |
| laropiprant | for counteracting niacin-induced flushing | PTGDR |
| niacin | antidyslipidaemic agent | GPR109A |
| niacin | antidyslipidaemic agent | GPR109B |
| niacin | antidyslipidacmic agent | NNMT |
| niacin | antidyslipidaemic agent | QPRT |
| laropiprant | for counteracting niacin-induced flushing | PTGDR |
| niacin | antidyslipidaemic agent | GPR109A |
| niacin | antidyslipidaemic agent | GPR109B |
| niacin | antidyslipidaemic agent | NNMT |
| niacin | antidyslipidaemic agent | QPRT |
| simvastatin | anticholesterolaemic agent | HMGCR |
| MK-1775 | antineoplastic agent | WEE1 |
| MK-2206 | antineoplastic agent | AKT1 |
| MK-2206 | antineoplastic agent | AKT2 |
| MK-2206 | antineoplastic agent | AKT3 |
| suvorexant | hypnotic | HCRTR1 |
| suvorexant | hypnotic | HCRTR2 |
| MK-4827 | antineoplastic agent | PARP1 |
| MK-4827 | antineoplastic agent | PARP2 |
| MKC-1 | antineoplastic agent | IPO11 |
| MKC-1 | antineoplastic agent | IPO13 |
| MKC-1 | antineoplastic agent | IPO4 |
| MKC-1 | antineoplastic agent | IPO7 |
| MKC-1 | antineoplastic agent | IPO8 |
| MKC-1 | antineoplastic agent | IPO9 |
| MKC-1 | antineoplastic agent | TUBB |
| MKC-1 | antineoplastic agent | TUBB1 |
| MLN-0415 | antiinflammatory agent | IKBKB |
| MLN-4924 | antincoplastic agent | UBA3 |
| MLN-8054 | antineoplastic agent | AUR2 |
| MLN-8237 | antineoplastic agent | AURKA |
| MLN-9708 | antineoplastic agent | PSMB1 |
| MLN-9708 | antineoplastic agent | PSMB2 |
| MLN-9708 | antineoplastic agent | PSMB5 |
| MLN-9708 | antineoplastic agent | PSMD1 |
| MLN-9708 | antineoplastic agent | PSMD2 |
| MN-201 | antineoplastic agent | VDR |
| MN-246 | for treatment of overactive bladder | ADRB3 |
| MN-305 | antidepressant, hypnotic | HTR1A |
| moclobemide | antidepressant | MAOA |
| modafinil | central nervous system stimulant | SLC6A3 |
| Modufolin | antineoplastic agent | TYMS |
| formoterol | antiasthmatic agent | ADRB2 |
| mometasone | antiinflammatory agent, glucocorticoid | NR3C1 |
| montelukast | antiasthmatic agent | CYSLTR1 |
| morphine | analgesic | OPRD1 |
| morphine | analgesic | OPRK1 |
| morphine | analgesic | OPRM1 |
| morphine | analgesic | OPRK1 |
| morphine | analgesic | OPRK1 |
| morphine | analgesic | OPRK1 |
| dextromethorphan | analgesic | GRIN3A |
| dextromethorphan | analgesic | SIGMAR1 |
| morphine | analgesic | OPRD1 |
| morphine | analgesic | OPRK1 |
| morphine | analgesic | OPRM1 |
| morphine | analgesic | OPRD1 |
| morphine | analgesic | OPRK1 |
| morphine | analgesic | OPRM1 |
| naltrexone | analgesic | OPRD1 |
| naltrexone | analgesic | OPRK1 |
| naltrexone | analgesic | OPRM1 |
| naltrexone | analgesic | SIGMAR1 |
| mosapride | for treatment of Gastrointestinal reflux disease | HTR4 |
| | (GERD) | |
| motesanib | antineoplastic agent | FLT1 |
| motesanib | antineoplastic agent | FLT4 |
| motesanib | antineoplastic agent | KDR |
| motesanib | antineoplastic agent | KIT |
| motesanib | antineoplastic agent | PDGFRA |
| motesanib | antineoplastic agent | PDGFRB |
| motexafin gadolinium | antineoplastic agent | RRM1 |
| motexafin gadolinium | antineoplastic agent | RRM2 |
| motexafin gadolinium | antineoplastic agent | RRM2B |
| motexafin gadolinium | antineoplastic agent | TXNRD1 |
| motexafin gadolinium | antineoplastic agent | TXNRD2 |
| motexafin gadolinium | antineoplastic agent | TXNRD3 |
| morphine | analgesic | OPRD1 |
| morphine | analgesic | OPRK1 |
| morphine | analgesic | OPRM1 |
| oxycodone | analgesic | OPRM1 |
| oxycodone | analgesic | OPRM1 |
| oxycodone | analgesic | OPRM1 |
| plerixafor | antineoplastic agent | CXCR4 |
| MP0112 | for treatment of diabetic retinopathy | FLT1 |
| MP0112 | for treatment of diabetic retinopathy | KDR |
| amuvatinib | antineoplastic agent | FLT3 |
| amuvatinib | antineoplastic agent | KIT |
| amuvatinib | antineoplastic agent | MET |
| amuvatinib | antineoplastic agent | PDGFRA |
| amuvatinib | antineoplastic agent | PDGFRB |
| amuvatinib | antineoplastic agent | RAD51 |
| amuvatinib | antineoplastic agent | RET |
| MPC-0920 | antithrombotic | F2 |
| MPI-674 | for treatment of abnormal uterine bleeding | CYP19A1 |
| | (AUB) | |
| MPI-676 | for treatment of endometriosis | CYP19A1 |
| nitroglycerin | for treatment of Raynaud's disease | NPR1 |
| MRX-4 | antiinflammatory agent | PLA2G3 |
| MRX-6 | antiinflammatory agent | PLA2G3 |
| mitoglitazone | antidiabetic | PPARG |
| talniflumate | for treatment of cystic fibrosis | CLCA1 |
| MSX-122 | antineoplastic agent | CXCR4 |
| metoclopramide | antimigraine agent | CHRM1 |
| metoclopramide | antimigraine agent | DRD2 |
| naproxen | antimigraine agent | PTGS1 |
| naproxen | antimigraine agent | PTGS2 |
| dihydroergotamine | antimigraine agent | HTR1B |
| dihydroergotamine | antimigraine agent | HTR1D |
| naproxen | antimigraine agent | PTGS1 |
| naproxen | antimigraine agent | PTGS2 |
| sumatriptan | antimigraine agent | HTR1A |
| sumatriptan | antimigraine agent | HTR1B |
| sumatriptan | antimigraine agent | HTR1D |
| sumatriptan | antimigraine agent | HTR1F |
| doxorubicin | antineoplastic agent | TOP2A |
| isothiourea | antihypertensive agent | NOS1 |
| isothiourea | antihypertensive agent | NOS2 |
| isothiourea | antihypertensive agent | NOS3 |
| muraglitazar | antidiabetic | PPARA |
| muraglitazar | antidiabetic | PPARG |
| mycophenolic acid | immunosuppressant | IMPDH1 |
| mycophenolic acid | immunosuppressant | IMPDH2 |
| MPC-3100 | antineoplastic agent | HSP90AA1 |
| MPC-3100 | antineoplastic agent | HSP90AB1 |
| docetaxel | antineoplastic agent | BCL2 |
| docetaxel | antineoplastic agent | TUBB1 |
| nabilone | antiemetic | CNR1 |
| nabilone | antiemetic | CNR2 |
| nalbuphine | analgesic | OPRD1 |
| nalbuphine | analgesic | OPRK1 |
| nalbuphine | analgesic | OPRM1 |
| nalmefene | smoking-cessation agent, for treatment of | OPRD1 |
| | addiction | |
| nalmefene | smoking-cessation agent, for treatment of | OPRK1 |
| | addiction | |
| nalmefene | smoking-cessation agent, for treatment of | OPRM1 |
| | addiction | |
| memantine | for treatment of Alzheimer's disease | GRIN2A |
| memantine | for treatment of Alzheimer's disease | GRIN2B |
| memantine | for treatment of Alzheimer's disease | GRIN3A |
| diclofenac | NSAID | PTGS1 |
| diclofenac | NSAID | PTGS2 |
| Naproxcinod | NSAID | GUCY1A2 |
| Naproxcinod | NSAID | PTGS1 |
| Naproxcinod | NSAID | PTGS2 |
| esomeprazole | Proton pump inhibitor | ATP4A |
| naproxen | NSAID | PTGS1 |
| naproxen | NSAID | PTGS2 |
| naproxen etemesil | NSAID | PTGS1 |
| naproxen etemesil | NSAID | PTGS2 |
| naratriptan | antimigraine agent | HTR1A |
| naratriptan | antimigraine agent | HTR1B |
| naratriptan | antimigraine agent | HTR1D |
| naratriptan | antimigraine agent | HTR1F |
| ketamine | analgesic | GRIN3A |
| ketamine | analgesic | GRIN3A |
| Nav 1.7 blocker | analgesic | SCN9A |
| NB-1011 | antineoplastic agent | TYMS |
| NBI-56418 | antineoplastic agent | GNRHR |
| NBI-98854 | antipsychotic agent | SLC18A2 |
| NCX 1510 | antiallergy agent | GUCY1A2 |
| NCX 1510 | antiallergy agent | HRH1 |
| NCX 4016 | antithrombotic | GUCY1A2 |
| NCX 4016 | antithrombotic | PTGS1 |
| NCX 4016 | antithrombotic | PTGS2 |
| carbidopa | antiparkinson agent | DDC |
| nebivolol | antihypertensive agent | ADRB1 |
| nelarabine | antineoplastic agent | POLA1 |
| nepicastat | for treatment of addiction, for treatment of post- | DBH |
| | traumatic stress disorder | |
| neramexane | for treatment of Alzheimer's disease | GRIN2A |
| neramexane | for treatment of Alzheimer's disease | GRIN2B |
| neramexane | for treatment of Alzheimer's disease | GRIN3A |
| neratinib | antineoplastic agent | EGFR |
| neratinib | antineoplastic agent | ERBB2 |
| ethinyl estradiol | contraceptive | ESR1 |
| progestin | contraceptive | PGR |
| Neu-2000 | cardioprotectant | GRIN1 |
| Neu-2000 | cardioprotectant | GRIN2A |
| Neu-2000 | cardioprotectant | GRIN2B |
| Neu-2000 | cardioprotectant | GRIN2C |
| Neu-2000 | cardioprotectant | GRIN2D |
| Neu-2000 | cardioprotectant | GRIN3A |
| Neu-2000 | cardioprotectant | GRIN3B |
| rotigotine | antiparkinson agent | DRD2 |
| rotigotine | antiparkinson agent | DRD3 |
| rotigotine | antiparkinson agent | DRD4 |
| sorafenib | antineoplastic agent | BRAF |
| sorafenib | antineoplastic agent | FLT3 |
| sorafenib | antineoplastic agent | FLT4 |
| sorafenib | antineoplastic agent | KDR |
| sorafenib | antineoplastic agent | KIT |
| sorafenib | antineoplastic agent | PDGFRB |
| sorafenib | antineoplastic agent | RAF1 |
| NG2-73 | hypnotic | GABRA2 |
| NG2-73 | hypnotic | GABRA3 |
| NG2-73 | hypnotic | GABRA5 |
| NG2-73 | hypnotic | GABRA6 |
| NG2-73 | hypnotic | GABRB1 |
| NG2-73 | hypnotic | GABRB1 |
| NG2-73 | hypnotic | GABRB2 |
| NG2-73 | hypnotic | GABRB2 |
| NG2-73 | hypnotic | GABRB3 |
| NG2-73 | hypnotic | GABRD |
| NG2-73 | hypnotic | GABRD |
| NG2-73 | hypnotic | GABRE |
| NG2-73 | hypnotic | GABRG1 |
| NG2-73 | hypnotic | GABRG2 |
| NG2-73 | hypnotic | GABRG3 |
| NG2-73 | hypnotic | GABRG3 |
| NG2-73 | hypnotic | GABRP |
| NG2-73 | hypnotic | GABRQ |
| NG2-73 | hypnotic | GABRR2 |
| NGD-4715 | appetite suppressant | MCHR1 |
| NGD-8243 | analgesic | TRPV1 |
| NGX267 | for treatment of dry mouth | CHRM1 |
| niacin receptor agonist | antiatherosclerotic agent | HCAR2 |
| niacin receptor agonist | antiatherosclerotic agent | HCAR3 |
| NIC5-15 | for treatment of Alzheimer's disease | APH1A |
| NIC5-15 | for treatment of Alzheimer's disease | PSENEN |
| nilotinib | antineoplastic agent | ABL1 |
| nitisinone | for treatment of restlegs legs syndrome, for | HPD |
| | treatment of hereditary tyrosinemia type 1 (HT-1) | |
| PEG-irinotecan | antineoplastic agent | TOP1 |
| PEG-irinotecan | antineoplastic agent | TOP1MT |
| PEG-docetaxel | antineoplastic agent | BCL2 |
| PEG-docetaxel | antineoplastic agent | TUBB1 |
| PEG-naloxol | for treatment of opioid-induced constipation | OPRM1 |
| NM-702 | for treatment of intermittent claudication | PDE3A |
| NM-702 | for treatment of intermittent claudication | PDE3B |
| hydromorphone | analgesic | OPRD1 |
| hydromorphone | analgesic | OPRK1 |
| hydromorphone | analgesic | OPRM1 |
| NMS-1116354 | antineoplastic agent | CDC7 |
| NNZ-2566 | neuroprotectant | IGF1 |
| ethinyl estradiol | contraceptive | ESR1 |
| norelgestromin | contraceptive | ESR1 |
| norelgestromin | contraceptive | PGR |
| noscapine | antineoplastic agent | HIF1A |
| latanoprost | for treatment of glaucoma | PTGFR |
| Cyclosporine A | immunosuppressant, opthalmological agent | CAMLG |
| Cyclosporine A | immunosuppressant, opthalmological agent | PPP3R2 |
| sumatriptan | antimigraine agent | HTR1A |
| sumatriptan | antimigraine agent | HTR1B |
| sumatriptan | antimigraine agent | HTR1D |
| sumatriptan | antimigraine agent | HTR1F |
| 17-beta estradiol | opthalmological agent | ESR1 |
| 17-beta estradiol | opthalmological agent | ESR2 |
| Fluoxetine | for treatment of autism | HTR2A |
| Fluoxetine | for treatment of autism | SLC6A4 |
| NPS-2143 | antiosteoporotic agent | CASR |
| diazepam | anticonvulsant | GABRA1 |
| diazepam | anticonvulsant | GABRA2 |
| diazepam | anticonvulsant | GABRA3 |
| diazepam | anticonvulsant | GABRA5 |
| diazepam | anticonvulsant | GABRB1 |
| diazepam | anticonvulsant | GABRB2 |
| diazepam | anticonvulsant | GABRB3 |
| diazepam | anticonvulsant | GABRD |
| diazepam | anticonvulsant | GABRE |
| diazepam | anticonvulsant | GABRG1 |
| diazepam | anticonvulsant | GABRG2 |
| diazepam | anticonvulsant | GABRG3 |
| diazepam | anticonvulsant | GABRP |
| diazepam | anticonvulsant | GABRQ |
| diazepam | anticonvulsant | GABRR1 |
| diazepam | anticonvulsant | GABRR2 |
| diazepam | anticonvulsant | GABRR3 |
| NRM8499 | for treatment of Alzheimer's disease | APP |
| NRP290 | analgesic | OPRD1 |
| NRP290 | analgesic | OPRK1 |
| NRP290 | analgesic | OPRM1 |
| triiodothyronine (T3) | hormone replacement | THRA |
| triiodothyronine (T3) | hormone replacement | THRB |
| NRX-5183 | hematopoietic agent | RARA |
| NS-304 | antihypertensive agent | PTGIR |
| NSD-644 | analgesic, antidepressant | SLC6A2 |
| NSD-644 | analgesic, antidepressant | SLC6A3 |
| NSD-644 | analgesic, antidepressant | SLC6A4 |
| NSD-788 | antidepressant | SLC6A2 |
| NSD-788 | antidepressant | SLC6A4 |
| allopurinol | for treatment of gout | XDH |
| NV-52 | antiinflammatory agent | TBXAS1 |
| glycopyrronium | for treatment of chronic obstructive pulmonary | CHRM1 |
| | disease (COPD) | |
| tizanidine | for treatment of skeletal muscular spasticity | ADRA2A |
| tizanidine | for treatment of skeletal muscular spasticity | ADRA2B |
| tizanidine | for treatment of skeletal muscular spasticity | ADRA2C |
| NXN-188 | antimigraine agent | HTR1B |
| NXN-188 | antimigraine agent | HTR1D |
| NXN-188 | antimigraine agent | NOS1 |
| ondansetron | antiemetic | HTR3A |
| paclitaxel | antineoplastic agent | BCL2 |
| paclitaxel | antineoplastic agent | TUBB1 |
| obatoclax | antineoplastic agent | BCL2 |
| betahistine | antiobesity agent | HRH1 |
| betahistine | antiobesity agent | HRH3 |
| obeticholic acid | for treatment of non-alcoholic fatty liver disease | NR1H4 |
| | (NAFLD), for treatment of Primary Biliary | |
| | Cirrhosis (PBC) | |
| OC000459 | antiallergy agent | PD2R2 |
| ocinaplon | anxiolytic | GABRA2 |
| ocinaplon | anxiolytic | GABRA3 |
| ocinaplon | anxiolytic | GABRA5 |
| ocinaplon | anxiolytic | GABRA6 |
| ocinaplon | anxiolytic | GABRB1 |
| ocinaplon | anxiolytic | GABRB1 |
| ocinaplon | anxiolytic | GABRB2 |
| ocinaplon | anxiolytic | GABRB2 |
| ocinaplon | anxiolytic | GABRB3 |
| ocinaplon | anxiolytic | GABRD |
| ocinaplon | anxiolytic | GABRD |
| ocinaplon | anxiolytic | GABRE |
| ocinaplon | anxiolytic | GABRG1 |
| ocinaplon | anxiolytic | GABRG2 |
| ocinaplon | anxiolytic | GABRG3 |
| ocinaplon | anxiolytic | GABRG3 |
| ocinaplon | anxiolytic | GABRP |
| ocinaplon | anxiolytic | GABRQ |
| ocinaplon | anxiolytic | GABRR2 |
| heparin | antithrombotic | F10 |
| heparin | antithrombotic | F2 |
| odanacatib | antiosteoporotic agent | CTSK |
| Oglemilast | antiasthmatic agent | PDE4A |
| Oglemilast | antiasthmatic agent | PDE4B |
| olanzapine | antipsychotic agent | ADRA1A |
| olanzapine | antipsychotic agent | ADRA1B |
| olanzapine | antipsychotic agent | ADRA2A |
| olanzapine | antipsychotic agent | ADRA2B |
| olanzapine | antipsychotic agent | ADRA2C |
| olanzapine | antipsychotic agent | CHRM1 |
| olanzapine | antipsychotic agent | CHRM2 |
| olanzapine | antipsychotic agent | CHRM3 |
| olanzapine | antipsychotic agent | CHRM4 |
| olanzapine | antipsychotic agent | CHRM5 |
| olanzapine | antipsychotic agent | DRD1 |
| olanzapine | antipsychotic agent | DRD2 |
| olanzapine | antipsychotic agent | DRD3 |
| olanzapine | antipsychotic agent | DRD4 |
| olanzapine | antipsychotic agent | DRD5 |
| olanzapine | antipsychotic agent | HRH1 |
| olanzapine | antipsychotic agent | HTR1A |
| olanzapine | antipsychotic agent | HTR1B |
| olanzapine | antipsychotic agent | HTR1D |
| olanzapine | antipsychotic agent | HTR1E |
| olanzapine | antipsychotic agent | HTR2A |
| olanzapine | antipsychotic agent | HTR2C |
| olanzapine | antipsychotic agent | HTR3A |
| olanzapine | antipsychotic agent | HTR6 |
| olanzapine | antipsychotic agent | HTR7 |
| fluoxetine | antidepressant, for treatment of bipolar disorder | SLC6A4 |
| olanzapine | antidepressant, for treatment of bipolar disorder | ADRA1A |
| olanzapine | antidepressant, for treatment of bipolar disorder | ADRA1B |
| olanzapinc | antidepressant, for treatment of bipolar disorder | ADRA2A |
| olanzapine | antidepressant, for treatment of bipolar disorder | ADRA2B |
| olanzapine | antidepressant, for treatment of bipolar disorder | ADRA2C |
| olanzapine | antidepressant, for treatment of bipolar disorder | CHRM1 |
| olanzapine | antidepressant, for treatment of bipolar disorder | CHRM2 |
| olanzapine | antidepressant, for treatment of bipolar disorder | CHRM3 |
| olanzapine | antidepressant, for treatment of bipolar disorder | CHRM4 |
| olanzapine | antidepressant, for treatment of bipolar disorder | CHRM5 |
| olanzapine | antidepressant, for treatment of bipolar disorder | DRD1 |
| olanzapine | antidepressant, for treatment of bipolar disorder | DRD2 |
| olanzapine | antidepressant, for treatment of bipolar disorder | DRD3 |
| olanzapine | antidepressant, for treatment of bipolar disorder | DRD4 |
| olanzapine | antidepressant, for treatment of bipolar disorder | DRD5 |
| olanzapine | antidepressant, for treatment of bipolar disorder | HRH1 |
| olanzapine | antidepressant, for treatment of bipolar disorder | HTR1A |
| olanzapine | antidepressant, for treatment of bipolar disorder | HTR1B |
| olanzapine | antidepressant, for treatment of bipolar disorder | HTR1D |
| olanzapine | antidepressant, for treatment of bipolar disorder | HTR1E |
| olanzapine | antidepressant, for treatment of bipolar disorder | HTR2A |
| olanzapine | antidepressant, for treatment of bipolar disorder | HTR2C |
| olanzapine | antidepressant, for treatment of bipolar disorder | HTR3A |
| olanzapine | antidepressant, for treatment of bipolar disorder | HTR6 |
| olanzapine | antidepressant, for treatment of bipolar disorder | HTR7 |
| olesoxime | for treatment of motor neuron disease | TSPO |
| olesoxime | for treatment of motor neuron disease | VDAC1 |
| olesoxime | for treatment of motor neuron disease | VDAC2 |
| olesoxime | for treatment of motor neuron disease | VDAC3 |
| olmesartan | antihypertensive agent | AGTR1 |
| olmesartan | for treatment of glaucoma | AGTR1 |
| olopatadine | antiallergy agent | HRH1 |
| omacetaxine mepesuccinate | antineoplastic agent | Ribosome A-site |
| ombrabulin | antineoplastic agent | TUBB1 |
| omecamtiv mecarbil | for treatment of heart failure | Cardiac Mysoin |
| omeprazole | Proton pump inhibitor | ATP4A |
| omeprazole | Proton pump inhibitor | ATP4A |
| omeprazole | Proton pump inhibitor | ATP4A |
| omigapil | antiparkinson agent, for treatment of | GAPDA |
| | amyotrophic lateral sclerosis (ALS) | |
| omigapil | antiparkinson agent, for treatment of | SIAH1 |
| | amyotrophic lateral sclerosis (ALS) | |
| amitriptyline | analgesic | HTR2A |
| amitriptyline | analgesic | HTR2A |
| amitriptyline | analgesic | SLC6A2 |
| amitriptyline | analgesic | SLC6A2 |
| amitriptyline | analgesic | SLC6A4 |
| amitriptyline | analgesic | SLC6A4 |
| ketoprofen | NSAID | PTGS1 |
| ketoprofen | NSAID | PTGS1 |
| ketoprofen | NSAID | PTGS2 |
| ketoprofen | NSAID | PTGS2 |
| oxymetazoline | analgesic | ADRA1A |
| oxymetazoline | analgesic | ADRA1A |
| oxymetazoline | analgesic | ADRA2A |
| oxymetazoline | analgesic | ADRA2A |
| rigosertib | antineoplastic agent | PIK3CA |
| rigosertib | antineoplastic agent | PIK3CB |
| rigosertib | antineoplastic agent | PIK3CD |
| rigosertib | antineoplastic agent | PLK1 |
| paclitaxel | antineoplastic agent | BCL2 |
| paclitaxel | antineoplastic agent | TUBB1 |
| ondansetron | antiemetic | HTR3A |
| oprozomib | antineoplastic agent | PSMB1 |
| oprozomib | antineoplastic agent | PSMB2 |
| oprozomib | antineoplastic agent | PSMB5 |
| oprozomib | antineoplastic agent | PSMD1 |
| oprozomib | antineoplastic agent | PSMD2 |
| paclitaxel | antineoplastic agent | BCL2 |
| paclitaxel | antineoplastic agent | TUBB1 |
| OPC-28326 | vasodilator | ADRA2B |
| OPC-28326 | vasodilator | ADRA2C |
| OPC-34712 | antidepressant | DRD2 |
| OPC-34712 | antidepressant | HTR1A |
| OPC-34712 | antidepressant | HTR2A |
| OPC-34712 | antidepressant | HTR7 |
| OPC-51803 | for treatment of incontinence | AVPR2 |
| doxycyklin | for treatment of dental disease | MMP8 |
| estrogen | contraceptive, for treatment of female sexual | ESR1 |
| | dysfunction | |
| estrogen | contraceptive, for treatment of female sexual | ESR2 |
| | dysfunction | |
| progestogen | contraceptive, for treatment of female sexual | PGR |
| | dysfunction | |
| estriol E3 | for treatment of multiple sclerosis | ESR1 |
| estriol E3 | for treatment of multiple sclerosis | ESR2 |
| paclitaxel | antineoplastic agent | BCL2 |
| paclitaxel | antincoplastic agent | TUBB1 |
| lidocaine | anesthetic | SCN10A |
| lidocaine | anesthetic | SCN5A |
| lidocaine | anesthetic | SCN9A |
| prilocaine | anesthetic | SCN5A |
| olanzapine | antipsychotic agent | ADRA1A |
| olanzapine | antipsychotic agent | ADRA1B |
| olanzapine | antipsychotic agent | ADRA2A |
| olanzapine | antipsychotic agent | ADRA2B |
| olanzapine | antipsychotic agent | ADRA2C |
| olanzapine | antipsychotic agent | CHRM1 |
| olanzapine | antipsychotic agent | CHRM2 |
| olanzapine | antipsychotic agent | CHRM3 |
| olanzapine | antipsychotic agent | CHRM4 |
| olanzapine | antipsychotic agent | CHRM5 |
| olanzapine | antipsychotic agent | DRD1 |
| olanzapine | antipsychotic agent | DRD2 |
| olanzapine | antipsychotic agent | DRD3 |
| olanzapine | antipsychotic agent | DRD4 |
| olanzapine | antipsychotic agent | DRD5 |
| olanzapine | antipsychotic agent | HRH1 |
| olanzapine | antipsychotic agent | HTR1A |
| olanzapine | antipsychotic agent | HTR1B |
| olanzapine | antipsychotic agent | HTR1D |
| olanzapine | antipsychotic agent | HTR1E |
| olanzapinc | antipsychotic agent | HTR2A |
| olanzapine | antipsychotic agent | HTR2C |
| olanzapine | antipsychotic agent | HTR3A |
| olanzapine | antipsychotic agent | HTR6 |
| olanzapine | antipsychotic agent | HTR7 |
| zonisamide | antipsychotic agent | CACNA1G |
| zonisamide | antipsychotic agent | CACNA1H |
| zonisamide | antipsychotic agent | CACNA1I |
| zonisamide | antipsychotic agent | SCN11A |
| zonisamide | antipsychotic agent | SCN1A |
| zonisamide | antipsychotic agent | SCN1B |
| zonisamide | antipsychotic agent | SCN2A |
| zonisamide | antipsychotic agent | SCN2B |
| zonisamide | antipsychotic agent | SCN3A |
| zonisamide | antipsychotic agent | SCN3B |
| zonisamide | antipsychotic agent | SCN4A |
| zonisamide | antipsychotic agent | SCN4B |
| zonisamide | antipsychotic agent | SCN5A |
| zonisamide | antipsychotic agent | SCN9A |
| orlistat | antiobesity agent | FASN |
| orlistat | antiobesity agent | LPL |
| orlistat | antiobesity agent | PNLIP |
| ortataxel | antineoplastic agent | BCL2 |
| ortataxel | antineoplastic agent | TUBB1 |
| orteronel | antineoplastic agent | CYP17A1 |
| OSI-027 | antineoplastic agent | MTOR |
| OSI-461 | antineoplastic agent | PDE5A |
| OSI-7904L | antineoplastic agent | TYMS |
| OSI-906 | antineoplastic agent | IGF1R |
| OSI-930 | antineoplastic agent | KDR |
| ospemifene | for treatment of postmenopausal vaginal atrophy | ESR1 |
| ospemifene | for treatment of postmenopausal vaginal atrophy | ESR2 |
| enobosarm | hormone replacement | AR |
| OT-730 | for treatment of glaucoma | ADRB1 |
| OT-730 | for treatment of glaucoma | ADRB2 |
| otamixaban | antithrombotic | F10 |
| dexamcthasonc | antiinflammatory agent, glucocorticoid, for | NR3C1 |
| | treatment of Meniere's disease | |
| famotidine | acid reducer | HRH2 |
| omeprazole | Proton pump inhibitor | ATP4A |
| zolpidem | hypnotic | GABRA1 |
| zolpidem | hypnotic | GABRA2 |
| zolpidem | hypnotic | GABRA3 |
| OX914 | antiallergy agent | PDE4A |
| OX914 | antiallergy agent | PDE4B |
| oxandrolone | anabolic agent | AR |
| oxcarbazepine | anticonvulsant | SCN5A |
| combretastatin A1 di-phosphate | antineoplastic agent | TUBB1 |
| oxycodone | analgesic | OPRD1 |
| oxycodone | analgesic | OPRK1 |
| oxycodone | analgesic | OPRM1 |
| niacin | substance abuse deterrant | GPR109A |
| niacin | substance abuse deterrant | GPR109B |
| niacin | substance abuse deterrant | NNMT |
| niacin | substance abuse deterrant | QPRT |
| oxycodone | analgesic | OPRD1 |
| oxycodone | analgesic | OPRK1 |
| oxycodone | analgesic | OPRM1 |
| oxycodone | analgesic | OPRD1 |
| oxycodone | analgesic | OPRK1 |
| oxycodone | analgesic | OPRM1 |
| oxymorphone | analgesic | OPRD1 |
| oxymorphone | analgesic | OPRM1 |
| P-552 | for treatment of dry mouth | ACCN2 |
| P-552 | for treatment of dry mouth | ACCN3 |
| P-552 | for treatment of dry mouth | ACCN4 |
| P-552 | for treatment of dry mouth | ASIC2 |
| P-552 | for treatment of dry mouth | SCNN1A |
| P-552 | for treatment of dry mouth | SCNN1B |
| P-552 | for treatment of dry mouth | SCNN1D |
| P-552 | for treatment of dry mouth | SCNN1G |
| acetylsalicylic acid | NSAID | PTGS1 |
| acctylsalicylic acid | NSAID | PTGS2 |
| omeprazole | Proton pump inhibitor | ATP4A |
| paclitaxel | antineoplastic agent | BCL2 |
| paclitaxel | antineoplastic agent | TUBB1 |
| paclitaxel | antineoplastic agent | BCL2 |
| paclitaxel | antineoplastic agent | TUBB1 |
| paclitaxel | for treatment of peripheral arterial disease | BCL2 |
| | (PAD) | |
| paclitaxel | for treatment of peripheral arterial disease | TUBB1 |
| | (PAD) | |
| pagoclone | for treatment of premature ejaculation, for | GABRA2 |
| | treatment of persistant stuttering | |
| pagoclonc | for treatment of premature cjaculation, for | GABRB2 |
| | treatment of persistant stuttering | |
| paliperidone | antipsychotic agent | DRD2 |
| paliperidone | antipsychotic agent | HTR2A |
| Palomid 529 | for treatment of age-related macular | MTOR |
| | degeneration | |
| Palonosetron | antiemetic | HTR3A |
| Panobinostat | antineoplastic agent | HDAC1 |
| Panobinostat | antineoplastic agent | HDAC10 |
| Panobinostat | antineoplastic agent | HDAC11 |
| Panobinostat | antineoplastic agent | HDAC2 |
| Panobinostat | antineoplastic agent | HDAC3 |
| Panobinostat | antineoplastic agent | HDAC4 |
| Panobinostat | antineoplastic agent | HDAC5 |
| Panobinostat | antineoplastic agent | HDAC6 |
| Panobinostat | antineoplastic agent | HDAC7A |
| Panobinostat | antineoplastic agent | HDAC8 |
| Panobinostat | antineoplastic agent | HDAC9 |
| pantoprazole | Proton pump inhibitor | ATP4A |
| pardoprunox | antiparkinson agent | ADRA1A |
| pardoprunox | antiparkinson agent | ADRA2A |
| pardoprunox | antiparkinson agent | DRD2 |
| pardoprunox | antiparkinson agent | DRD3 |
| pardoprunox | antiparkinson agent | DRD4 |
| pardoprunox | antiparkinson agent | HTR1A |
| pardoprunox | antiparkinson agent | HTR7 |
| parecoxib | antiinflammatory agent, NSAID | PTGS2 |
| paricalcitol | for treatment of hyperparathyroidism | VDR |
| paroxetine | antidepressant | SLC6A4 |
| Pazopanib | antineoplastic agent | FLT1 |
| Pazopanib | antineoplastic agent | FLT4 |
| Pazopanib | antineoplastic agent | KDR |
| bleomycin | antineoplastic agent | LIG1 |
| CRA-024781 | antineoplastic agent | HDAC1 |
| CRA-024781 | antineoplastic agent | HDAC10 |
| CRA-024781 | antineoplastic agent | HDAC2 |
| CRA-024781 | antineoplastic agent | HDAC3 |
| CRA-024781 | antineoplastic agent | HDAC6 |
| ibrutinib | antineoplastic agent | BTK |
| PD-6735 | hypnotic | MTNR1A |
| PD-6735 | hypnotic | MTNR1B |
| 10-propargyl-10- | antineoplastic agent | DHFR |
| deazaaminopterin | | |
| PEG-camptothecin | antineoplastic agent | TOP1 |
| pentosan polysulfate | for symptomatic treatment of bladder pain or | FGF1 |
| | discomfort associated with interstitial cystitis | |
| pentosan polysulfate | for symptomatic treatment of bladder pain or | FGF2 |
| | discomfort associated with interstitial cystitis | |
| pentosan polysulfate | for symptomatic treatment of bladder pain or | FGF4 |
| | discomfort associated with interstitial cystitis | |
| pentostatin | antineoplastic agent | ADA |
| pentoxifylline | for treatment of amyotrophic lateral sclerosis | ADORA1 |
| | (ALS) | |
| pentoxifylline | for treatment of amyotrophic lateral sclerosis | ADORA2B |
| | (ALS) | |
| pentoxifylline | for treatment of amyotrophic lateral sclerosis | PDE4A |
| | (ALS) | |
| pentoxifylline | for treatment of amyotrophic lateral sclerosis | PDE4B |
| | (ALS) | |
| pentoxifylline | for treatment of amyotrophic lateral sclerosis | PDE5A |
| | (ALS) | |
| ingenol Mebutate | for treatment of actinic keratosis, antineoplastic | PKN1 |
| | agent | |
| ingenol Mebutate | for treatment of actinic keratosis, antineoplastic | PKN2 |
| | agent | |
| ingenol Mebutate | for treatment of actinic keratosis, antineoplastic | PRKCA |
| | agent | |
| ingenol Mebutate | for treatment of actinic keratosis, antineoplastic | PRKCB1 |
| | agent | |
| ingenol Mcbutatc | for treatment of actinic keratosis, antineoplastic | PRKCD |
| | agent | |
| ingenol Mebutate | for treatment of actinic keratosis, antineoplastic | PRKCE |
| | agent | |
| ingenol Mebutate | for treatment of actinic keratosis, antineoplastic | PRKCG |
| | agent | |
| ingenol Mebutate | for treatment of actinic keratosis, antineoplastic | PRKCH |
| | agent | |
| ingenol Mebutate | for treatment of actinic keratosis, antineoplastic | PRKCI |
| | agent | |
| ingenol Mebutate | for treatment of actinic keratosis, antineoplastic | PRKCQ |
| | agent | |
| ingenol Mebutate | for treatment of actinic keratosis, antineoplastic | PRKCZ |
| | agent | |
| irinotecan | antineoplastic agent | TOP1 |
| irinotecan | antineoplastic agent | TOP1MT |
| perifosine | antineoplastic agent | AKT1 |
| perifosine | antineoplastic agent | AKT2 |
| perifosine | antineoplastic agent | AKT3 |
| PF-00610355 | bronchodilator | ADRB2 |
| PF-04554878 | antineoplastic agent | PTK2 |
| Dacomitinib | antineoplastic agent | EGFR |
| Dacomitinib | antineoplastic agent | ERBB2 |
| Dacomitinib | antineoplastic agent | ERBB4 |
| PG-490-88 | antineoplastic agent | NFKB1 |
| PG-490-88 | antineoplastic agent | NFKB2 |
| PG545 | antineoplastic agent | HPSE |
| PH-797804 | antiinflammatory agent, DMARD | MAPK11 |
| PH-797804 | antiinflammatory agent, DMARD | MAPK12 |
| PH-797804 | antiinflammatory agent, DMARD | MAPK13 |
| PH-797804 | antiinflammatory agent, DMARD | MAPK14 |
| phenoxodiol | antineoplastic agent | SPHK1 |
| phenoxodiol | antineoplastic agent | SPHK2 |
| phenserine | for treatment of Alzheimer's disease | ACHE |
| physostigmine | for treatment of dry mouth | ACHE |
| Pimavanserin | antiparkinson agent | HTR2A |
| pimecrolimus | antiinflammatory agent | MTOR |
| pioglitazone | antidiabetic | PPARG |
| metformin | antidiabetic | PRKAB1 |
| pioglitazone | antidiabetic | PPARG |
| pirfenidone | for treatment of fibrotic conditions | MAPK11 |
| pirfenidone | for treatment of fibrotic conditions | MAPK12 |
| pirfenidone | for treatment of fibrotic conditions | MAPK13 |
| pirfenidone | for treatment of fibrotic conditions | MAPK14 |
| pitavastatin | anticholesterolaemic agent | HMGCR |
| PL37 | analgesic, neuropathic pain | ANPEP |
| PL37 | analgesic, neuropathic pain | MME |
| clopidogrel | antithrombotic | P2RY12 |
| PLK-1 inhibitor | antineoplastic agent | PLK1 |
| vemurafenib | antineoplastic agent | BRAF |
| PMI-001 | antiinflammatory agent, DMARD | NR3C1 |
| naproxen | NSAID | PTGS1 |
| naproxen | NSAID | PTGS2 |
| omeprazole | Proton pump inhibitor | ATP4A |
| carmustine | antineoplastic agent | GSR |
| ponatinib | antineoplastic agent | ABL1 |
| ponatinib | antineoplastic agent | SRC |
| ponesimod | antiinflammatory agent, for treatment of multiple | S1PR1 |
| | sclerosis | |
| Posiphen | for treatment of Alzheimer's disease | APP |
| Posiphen | for treatment of Alzheimer's disease | BACE1 |
| Posiphen | for treatment of Alzheimer's disease | BACE2 |
| pozanicline | for treatment of Alzheimer's disease | CHRNA4 |
| pozanicline | for treatment of Alzheimer's disease | CHRNB2 |
| PPC-5650 | analgesic | ACCN2 |
| PPI-2458 | antineoplastic agent | METAP2 |
| PR-15 | antithrombotic | GP6 |
| prasterone | hormone supplement for increasing bone | AR |
| | mineral density in patients with systemic lupus | |
| | erythematosus | |
| prasugrel | antithrombotic | P2RY12 |
| fenofibrate | anticholesterolaemic agent | PPARA |
| pravastatin | anticholesterolaemic agent | HMGCR |
| prednisolone | antiinflammatory agent, corticosteroid | NR3C1 |
| prednisolone | antiinflammatory agent, corticosteroid | NR3C1 |
| pregabalin | analgesic, neuropathic pain, for treatment of | CACNA1A |
| | restlegs legs syndrome | |
| preladenant | antiparkinson agent | ADORA2A |
| pridopidine | for treatment of Huntington's disease | DRD2 |
| desvenlafaxine | for treatment of menopausal | SLC6A2 |
| | symptoms, antidepressant | |
| desvenlafaxine | for treatment of menopausal | SLC6A4 |
| | symptoms, antidepressant | |
| diclofenac | NSAID | PTGS1 |
| diclofenac | NSAID | PTGS2 |
| telapristone | for treatment of uterin fibroids and | PGR |
| | endometriosis | |
| progesterone | for reducing the risk of pre-term birth for women | PGR |
| | with short cervix a mid-pregnancy | |
| testosterone | hormone replacement | AR |
| eltrombopag | thrombopoietic | MPL |
| propafenone | antiarrythmic agent | KCNH2 |
| propafenone | antiarrythmic agent | SCN5A |
| propionyl-L-carnitine | for treatment of intermittent claudication | CPT1A |
| propionyl-L-carnitine | for treatment of intermittent claudication | CPT2 |
| propionyl-L-carnitine | for treatment of intermittent claudication | CRAT |
| propionyl-L-carnitine | for treatment of intermittent claudication | CROT |
| propionyl-L-carnitine | for treatment of intermittent claudication | SLC22A4 |
| propionyl-L-carnitine | for treatment of intermittent claudication | SLC22A5 |
| propionyl-L-carnitine | for treatment of intermittent claudication | SLC25A20 |
| propionyl-L-carnitine | for treatment of intermittent claudication | SLC25A29 |
| propofol | sedative | GABRB2 |
| propofol | sedative | GABRB3 |
| propofol | sedative | SCN2A |
| propofol | sedative | SCN4A |
| propofol | scdativc | GABRB2 |
| propofol | sedative | GABRB3 |
| propofol | sedative | SCN2A |
| propofol | sedative | SCN4A |
| OPC-14523 | antidepressant | HTR1A |
| OPC-14523 | antidepressant | PGRMC1 |
| OPC-14523 | antidepressant | SIGMAR1 |
| OPC-14523 | antidepressant | SLC6A4 |
| PRT062607 | antiinflammatory agent | SYK |
| prucalopride | motilitant | HTR4 |
| PRX-00023 | antidepressant, anxiolytic | HTR1A |
| PRX-07034 | antiobesity agent, nootropic | HTR6 |
| PRX-08066 | antihypertensive agent | HTR2B |
| PRX-3140 | for treatment of Alzheimer's disease | HTR4 |
| PS433540 | antihypertensive agent | AGTR1 |
| PS433540 | antihypertensive agent | AGTR2 |
| PS433540 | antihypertensive agent | EDNRA |
| lidocaine | for treatment of premature ejaculation | EGFR |
| lidocaine | for treatment of premature ejaculation | SCN10A |
| lidocaine | for treatment of premature ejaculation | SCN5A |
| prilocaine | for treatment of premature ejaculation | SCN5A |
| phenylephrine | for treatment of incontinence | ADRA1A |
| phenylephrine | for treatment of incontinence | ADRA1B |
| phenylephrine | for treatment of incontinence | ADRA1D |
| PSD-506 | for treatment of overactive bladder | CHRM2 |
| PSD-506 | for treatment of overactive bladder | CHRM3 |
| PSN357 | antidiabetic | PYGB |
| PSN357 | antidiabetic | PYGL |
| PSN357 | antidiabetic | PYGM |
| PSN602 | antiobesity agent | HTR1A |
| PSN602 | antiobesity agent | SLC6A2 |
| PSN602 | antiobesity agent | SLC6A3 |
| PSN602 | antiobesity agent | SLC6A4 |
| PSN821 | antidiabetic | GPR119 |
| glycopyrrolate | for treatment of chronic obstructive pulmonary | CHRM1 |
| | disorder (COPD) | |
| formoterol | for treatment of chronic obstructive pulmonary | ADRB2 |
| | disorder (COPD) | |
| glycopyrrolate | for treatment of chronic obstructive pulmonary | CHRM1 |
| | disorder (COPD) | |
| formoterol | for treatment of chronic obstructive pulmonary | ADRB2 |
| | disorder (COPD) | |
| PTC299 | antineoplastic agent | FLT1 |
| PTC299 | antineoplastic agent | FLT4 |
| PTC299 | antineoplastic agent | KDR |
| naltrexone | analgesic | OPRD1 |
| naltrexone | analgesic | OPRD1 |
| naltrexone | analgesic | OPRK1 |
| naltrexone | analgesic | OPRK1 |
| naltrexone | analgesic | OPRM1 |
| naltrexone | analgesic | OPRM1 |
| naltrexone | analgesic | SIGMAR1 |
| tramadol | analgesic | HTR2C |
| tramadol | analgesic | OPRK1 |
| tramadol | analgesic | OPRK1 |
| tramadol | analgesic | OPRM1 |
| tramadol | analgesic | OPRM1 |
| tramadol | analgesic | SLC6A2 |
| tramadol | analgesic | SLC6A2 |
| tramadol | analgesic | SLC6A4 |
| acetaminophen | analgesic | PTGS1 |
| acetaminophen | analgesic | PTGS1 |
| acetaminophen | analgesic | PTGS2 |
| acetaminophen | analgesic | PTGS2 |
| hydrocodone | analgesic | OPRD1 |
| hydrocodone | analgesic | OPRD1 |
| hydrocodone | analgesic | OPRM1 |
| hydrocodone | analgesic | OPRM1 |
| naltrexone | analgesic | OPRD1 |
| naltrexone | analgesic | OPRD1 |
| naltrexone | analgesic | OPRK1 |
| naltrexone | analgesic | OPRK1 |
| naltrexone | analgesic | OPRM1 |
| naltrexone | analgesic | OPRM1 |
| naltrexone | analgesic | SIGMAR1 |
| naltrexonc | analgesic | SIGMAR1 |
| naltrexone | analgesic | OPRD1 |
| naltrexone | analgesic | OPRK1 |
| naltrexone | analgesic | OPRM1 |
| oxycodone | analgesic | OPRD1 |
| oxycodone | analgesic | OPRK1 |
| oxycodone | analgesic | OPRM1 |
| naltrexone | analgesic | OPRD1 |
| naltrexone | analgesic | OPRK1 |
| naltrexone | analgesic | OPRM1 |
| Pumosetrag | motilitant | HTR3A |
| Pumosetrag | motilitant | HTR3B |
| Pumosetrag | motilitant | HTR3C |
| Pumosetrag | motilitant | HTR3D |
| Pumosetrag | motilitant | HTR3E |
| PW2101 | antihypertensive agent | ADRB2 |
| PX-12 | antineoplastic agent | TXN |
| PX-478 | antineoplastic agent | HIF1A |
| belinostat | antineoplastic agent | HDAC1 |
| belinostat | antineoplastic agent | HDAC10 |
| belinostat | antineoplastic agent | HDAC11 |
| belinostat | antineoplastic agent | HDAC2 |
| belinostat | antineoplastic agent | HDAC3 |
| belinostat | antineoplastic agent | HDAC4 |
| belinostat | antineoplastic agent | HDAC5 |
| belinostat | antineoplastic agent | HDAC6 |
| belinostat | antineoplastic agent | HDAC7A |
| belinostat | antineoplastic agent | HDAC8 |
| belinostat | antineoplastic agent | HDAC9 |
| PYM50028 | antiparkinson agent | GFRA1 |
| PYM50028 | antiparkinson agent | NGFR |
| PYM50028 | antiparkinson agent | NTRK1 |
| PYM50028 | antiparkinson agent | NTRK2 |
| quinapril | antihypertensive agent | ACE |
| glycopyrronium bromide | for treatment of chronic obstructive pulmonary | ADRB2 |
| | disorder (COPD) | |
| indacaterol | for treatment of chronic obstructive pulmonary | CHRM1 |
| | disorder (COPD) | |
| R112 | antiallergy agent | FCER1A |
| R112 | antiallergy agent | FCER1G |
| R112 | antiallergy agent | MS4A2 |
| R343 | antiallergy agent | SYK |
| R348 | antiinflammatory agent | JAK3 |
| R667 | for treatment of emphysema | RARA |
| R667 | for treatment of emphysema | RARB |
| R667 | for treatment of emphysema | RARG |
| R763 | antineoplastic agent | AURKA |
| R763 | antineoplastic agent | AURKB |
| R763 | antineoplastic agent | AURKC |
| RAD1901 | for treatment of postmenopausal symptoms | ESR1 |
| raltitrexed | antineoplastic agent | TYMS |
| ramelteon | for treatment of insomnia | MTNR1A |
| ramelteon | for treatment of insomnia | MTNR1B |
| ranolazine | antiallergy agent | SCN5A |
| ranolazine | antiallergy agent | SCN9A |
| ranirestat | for treatment of diabetic neuropathy | AKR1B1 |
| ranitidine | antiulcer agent | HRH2 |
| rasagiline | antiparkinson agent | MAOB |
| RC-8800 | for improving the antiproliferative and apoptotic | CYP46A1 |
| | properties of vitamin D3 | |
| RDEA119 | antineoplastic agent | MAPK1 |
| RDEA119 | antineoplastic agent | MAPK3 |
| regadenoson | diagnostic agent | ADORA2A |
| regorafenib | antineoplastic agent | KDR |
| regorafenib | antineoplastic agent | TEK |
| relacatib | antiosteoporotic agent | CTSK |
| eletriptan | antimigraine agent | HTR1D |
| remifentanil | analgesic | OPRM1 |
| Nalbuphine | analgesic | OPRD1 |
| Nalbuphine | analgesic | OPRK1 |
| Nalbuphine | analgesic | OPRM1 |
| naloxone | analgesic | OPRD1 |
| naloxone | analgesic | OPRK1 |
| naloxone | analgesic | OPRM1 |
| renzapride | for treatment of irritable bowel syndrome | HTR2A |
| renzapride | for treatment of irritable bowel syndrome | HTR2B |
| renzapride | for treatment of irritable bowel syndrome | HTR2C |
| renzapride | for treatment of irritable bowel syndrome | HTR3A |
| renzapride | for treatment of irritable bowel syndrome | HTR4 |
| repaglinide | antidiabetic | ABCC8 |
| ropinirol | antiparkinson agent | DRD2 |
| ropinirol | antiparkinson agent | DRD3 |
| resiniferatoxin | for treatment of interstitial | TRPV1 |
| | cystitis, antiincontinence agent | |
| Resminostat | antineoplastic agent | HDAC1 |
| Resminostat | antineoplastic agent | HDAC10 |
| Resminostat | antineoplastic agent | HDAC11 |
| Resminostat | antineoplastic agent | HDAC2 |
| Resminostat | antineoplastic agent | HDAC3 |
| Resminostat | antineoplastic agent | HDAC4 |
| Resminostat | antineoplastic agent | HDAC5 |
| Resminostat | antineoplastic agent | HDAC6 |
| Resminostat | antineoplastic agent | HDAC7A |
| Resminostat | antineoplastic agent | HDAC8 |
| Resminostat | antineoplastic agent | HDAC9 |
| Resveratrol | for treatment of herpes simplex virus 1 | PDE4B |
| Resveratrol | for treatment of herpes simplex virus l | PDE4D |
| retigabine | anticonvulsant | KCNQ1 |
| retigabine | anticonvulsant | KCNQ2 |
| retigabine | anticonvulsant | KCNQ3 |
| retigabine | anticonvulsant | KCNQ4 |
| retigabine | anticonvulsant | KCNQ5 |
| rEV131 | antiallergy agent | HRH4 |
| lenalidomide | antineoplastic agent | TNFSF11 |
| RG2833 | for treatment of Friedrich's ataxia | HDAC3 |
| RG3039 | for treatment of spinal muscular atrophy | DCPS |
| Ridaforolimus | antineoplastic agent | MTOR |
| riluzole | for treatment of ALS | SCN5A |
| riluzole | for treatment of ALS | SLC7A11 |
| rimcazole | antineoplastic agent | SIGMAR1 |
| Rimonabant | antiobesity agent | CNR1 |
| riociguat | antihypertensive agent | GUCY1A2 |
| riociguat | antihypertensive agent | GUCY1A3 |
| riociguat | antihypertensive agent | GUCY1B2 |
| riociguat | antihypertensive agent | GUCY1B3 |
| risedronate | antiosteoporotic agent | FDPS |
| Risperdal | antipsychotic agent | DRD2 |
| Risperdal | antipsychotic agent | HTR2A |
| rivaroxaban | antithrombotic | F10 |
| rivastigmine | for treatment of Alzheimer's disease | ACHE |
| rivastigmine | for treatment of Alzheimer's disease | BCHE |
| Rob 803 | antiinflammatory agent, DMARD | unknown |
| rocuronium | muscle relaxant | CHRM2 |
| rocuronium | muscle relaxant | CHRNA2 |
| rocuronium | muscle relaxant | HTR3A |
| rofecoxib | NSAID | PTGS2 |
| roflumilast | for treatment of chronic obstructive pulmonary | PDE4A |
| | disorder (COPD) | |
| roflumilast | for treatment of chronic obstructive pulmonary | PDE4B |
| | disorder (COPD) | |
| rolofylline | for treatment of congestive heart failure | ADORA1 |
| ronacaleret | antiiosteoporotic agent | CASR |
| ropivacaine | anestethic | SCN10A |
| glimepiride | antidiabetic | ABCC8 |
| glimepiride | antidiabetic | KCNJ1 |
| glimepiride | antidiabetic | KCNJ11 |
| rosiglitazone | antidiabetic | PPARG |
| metformin | antidiabetic | PRKAB1 |
| rosiglitazone | antidiabetic | PPARG |
| rosiglitazone | for treatment of Alzheimer's disease, antidiabetic | PPARG |
| ketorolac | antimigraine agent | PTGS1 |
| ketorolac | antimigraine agent | PTGS2 |
| bromovinyl deoxyuridine | antineoplastic agent | POLA1 |
| RPC1063 | for treatment of multiple sclerosis | S1PR1 |
| RPL-554 | bronchodilator | PDE3A |
| RPL-554 | bronchodilator | PDE3B |
| RPL-554 | bronchodilator | PDE4A |
| RPL-554 | bronchodilator | PDE4B |
| RTA 744 | antineoplastic agent | TOP2A |
| RTA 744 | antineoplastic agent | TOP2B |
| rubitecan | antineoplastic agent | TOP1 |
| ruboxistaurin | for treatment of diabetic neuropathy | PRKCB1 |
| RVX-208 | antiatherosclerotic agent | APOA1 |
| gimestat | antineoplastic agent | DPYD |
| tegafur | antineoplastic agent | TYMS |
| paclitaxel | antineoplastic agent | BCL2 |
| paclitaxel | antineoplastic agent | TUBB1 |
| SA4503 | antidepressant, neuroprotectant | SIGMAR1 |
| Safinamide | antiparkinson agent | CACNA1B |
| Safinamide | antiparkinson agent | CACNA2D1 |
| Safinamide | antiparkinson agent | CACNA2D2 |
| Safinamide | antiparkinson agent | CACNB3 |
| Safinamide | antiparkinson agent | CACNB4 |
| Safinamide | antiparkinson agent | MAOB |
| Safinamide | antiparkinson agent | SCN11A |
| Safinamide | antiparkinson agent | SCN11A |
| Safinamide | antiparkinson agent | SCN1A |
| Safinamide | antiparkinson agent | SCN2A |
| Safinamide | antiparkinson agent | SCN3A |
| Safinamide | antiparkinson agent | SCN4A |
| Safinamide | antiparkinson agent | SCN5A |
| Safinamide | antiparkinson agent | SCN7A |
| Safinamide | antiparkinson agent | SCN8A |
| Safinamide | antiparkinson agent | SCN9A |
| tetrahydrobiopterin | for treatment of phenolketonuria (PKU) | NOS3 |
| tetrahydrobiopterin | for treatment of phenolketonuria (PKU) | PAH |
| tetrahydrobiopterin | for treatment of phenolketonuria (PKU) | TH |
| tetrahydrobiopterin | for treatment of phenolketonuria (PKU) | TPH1 |
| SAR 1118 | antiinflammatory agent | ICAM1 |
| SAR 1118 | antiinflammatory agent | ITGAL |
| SAR 1118 | antiinflammatory agent | ITGB2 |
| saredutant | antidepressant, anxiolytic | TACR2 |
| nabilone | analgesic, neuropathic pain, for treatment of | CNR2 |
| | restlegs legs syndrome | |
| nabilone | analgesic, neuropathic pain, for treatment of | CNR2 |
| | restlegs legs syndrome | |
| Saxagliptin | antidiabetic | DPP4 |
| SB1518 | antineoplastic agent | JAK2 |
| SB-559448 | thrombopoietic agent | MPL |
| SB-681323 | antiinflammatory agent, DMARD | MAPK14 |
| firategrast | antiinflammatory agent | ITGA4 |
| firategrast | antiinflammatory agent | ITGB1 |
| pracinostat | antineoplastic agent | HDAC1 |
| pracinostat | antineoplastic agent | HDAC10 |
| pracinostat | antineoplastic agent | HDAC11 |
| pracinostat | antineoplastic agent | HDAC2 |
| pracinostat | antineoplastic agent | HDAC3 |
| pracinostat | antineoplastic agent | HDAC4 |
| pracinostat | antineoplastic agent | HDAC5 |
| pracinostat | antineoplastic agent | HDAC6 |
| pracinostat | antineoplastic agent | HDAC7A |
| pracinostat | antineoplastic agent | HDAC8 |
| pracinostat | antineoplastic agent | HDAC9 |
| SCH-527123 | for treatment of chronic obstructive pulmonary | CXCR1 |
| | disorder (COPD) | |
| SCH-527123 | for treatment of chronic obstructive pulmonary | CXCR2 |
| | disorder (COPD) | |
| talmapimod | antiinflammatory agent, DMARD | MAPK14 |
| SCY-635 | for treatment of hepatitis C | PP1A |
| SCY-635 | for treatment of hepatitis C | PP1D |
| scyllo-inositol | for treatment of Alzheimer's disease | APP |
| R-etodolac | antineoplastic agent | RXRA |
| selegiline | antidepressant | MAOB |
| selegiline | antiparkinson agent | MAOB |
| seletracetam | anticonvulsant | SV2A |
| selexipag | antihypertensive agent | PTGIR |
| seliciclib | antineoplastic agent | CDK2 |
| seliciclib | antineoplastic agent | CDK7 |
| seliciclib | antineoplastic agent | CDK9 |
| maraviroc | antiviral agent, HIV | CCR5 |
| eszopiclone | anxiolytic | GABRA1 |
| clavulanic acid | antidepressant | FOLH1 |
| SERTINDOLE | antipsychotic agent | ADRA1A |
| SERTINDOLE | antipsychotic agent | ADRA1B |
| SERTINDOLE | antipsychotic agent | ADRA1D |
| SERTINDOLE | antipsychotic agent | DRD2 |
| SERTINDOLE | antipsychotic agent | HTR2A |
| SERTINDOLE | antipsychotic agent | HTR2C |
| SERTINDOLE | antipsychotic agent | HTR6 |
| SERTINDOLE | antipsychotic agent | KCNH2 |
| salmeterol | bronchodilator | ADRB2 |
| Quetiapine | antipsychotic agent, antidepressant | DRD2 |
| Quetiapine | antipsychotic agent, antidepressant | HTR2A |
| Quetiapine | antipsychotic agent, antidepressant | HTR2B |
| Quetiapine | antipsychotic agent, antidepressant | HTR2C |
| Quetiapine | antipsychotic agent, antidepressant | HTR2C |
| SF1126 | antineoplastic agent | MTOR |
| SF1126 | antineoplastic agent | PIK3C3 |
| SF1126 | antineoplastic agent | PIK3CA |
| SF1126 | antineoplastic agent | PIK3CA |
| SF1126 | antineoplastic agent | PIK3CB |
| SF1126 | antineoplastic agent | PIK3CD |
| SF1126 | antineoplastic agent | PIK3CD |
| SF1126 | antineoplastic agent | PIK3CG |
| SF1126 | antineoplastic agent | PIK3CG |
| SF1126 | antineoplastic agent | PRKDC |
| SGI-1776 | antineoplastic agent | PIM1 |
| SGI-1776 | antineoplastic agent | PIM2 |
| SGI-1776 | antineoplastic agent | PIM3 |
| beclomethasone | antiinflammatory agent, glucocorticoid | NR3C1 |
| SGX523 | antineoplastic agent | MET |
| sibutramine | appetite suppressant | SLC6A2 |
| sibutramine | appetite suppressant | SLC6A3 |
| sibutramine | appetite suppressant | SLC6A4 |
| sildenafil | for treatment of erectile | PDE5A |
| | dysfucntion, antihypertensive agent | |
| doxepin | hypnotic | CHRM1 |
| doxepin | hypnotic | CHRM2 |
| doxepin | hypnotic | CHRM3 |
| doxepin | hypnotic | CHRM4 |
| doxepin | hypnotic | CHRM5 |
| doxepin | hypnotic | HRH1 |
| doxepin | hypnotic | HRH2 |
| doxepin | hypnotic | HTR2A |
| doxepin | hypnotic | HTR2B |
| doxepin | hypnotic | HTR2C |
| doxepin | hypnotic | SLC6A2 |
| doxepin | hypnotic | SLC6A4 |
| Silodosin | for treatment of BPH-related urinary symptoms | ADRA1A |
| sirolimus | for treatment of wct age-related macular | FKBP1A |
| | degeneration | |
| sirolimus | for treatment of wet age-related macular | MTOR |
| | degeneration | |
| sirolimus | immunosuppressant | FKBP1A |
| sirolimus | immunosuppressant | MTOR |
| Sitagliptin | antidiabetic | DPP4 |
| sivelestat | for treatment of acute lung injury associated with | ELA2 |
| | systemic inflammatory response syndrome | |
| | (SIRS) | |
| zaleplon | hypnotic | GABRA1 |
| zaleplon | hypnotic | TSPO |
| fluticasone | antiinflammatory agent, glucocorticoid | NR3C1 |
| formoterol | bronchodilator | ADRB2 |
| amphetamine | for treatment of cognitive dysfunction, for | SLC18A2 |
| | treatment of ADHD | |
| amphetamine | for treatment of cognitive dysfunction, for | SLC6A3 |
| | treatment of ADHD | |
| amphetamine | for treatment of cognitive dysfunction, for | TAAR1 |
| | treatment of ADHD | |
| dextroamphetamine | for treatment of ADHD | SLC18A2 |
| dextroamphetamine | for treatment of ADHD | SLC6A2 |
| dextroamphetamine | for treatment of ADHD | SLC6A3 |
| SLx-2101 | antihypertensive agent, for treatment of erectile | PDE5A |
| | dysfunction | |
| SLx-4090 | antidyslipidaemic agent | MTTP |
| SNS-032 | antineoplastic agent | CDK2 |
| SNS-032 | antineoplastic agent | CDK7 |
| SNS-032 | antineoplastic agent | CDK9 |
| SNS-314 | antineoplastic agent | AURKA |
| SNS-314 | antineoplastic agent | AURKB |
| SNX-5422 | antineoplastic agent | HSP90AA1 |
| SNX-5422 | antineoplastic agent | HSP90AB1 |
| sobetirome | antihypecholesterolemic agent | THRB |
| gamma hydroxybutyric acid | hypnotic | GABBR1 |
| gamma hydroxybutyric acid | hypnotic | GABBR2 |
| gamma hydroxybutyric acid | hypnotic | SLC5A2 |
| stibogluconate | antineoplastic agent | PTPN11 |
| levonorgestrel | contraceptive | ESR1 |
| levonorgestrel | contraceptive | PGR |
| levonorgestrel | contraceptive | SRD5A1 |
| solabegron | antidiabetic, for treatment of irritable bowel | ADRB3 |
| | syndrome, antiincontinence agent | |
| Solifenacin | for treatment of incontinence | CHRM1 |
| Solifenacin | for treatment of incontinence | CHRM2 |
| Solifenacin | for treatment of incontinence | CHRM3 |
| Solifenacin | for treatment of incontinence | CHRM4 |
| Solifenacin | for treatment of incontinence | CHRM5 |
| SOU-001 | for treatment of incontinence | ADRA1A |
| SOU-001 | for treatment of incontinence | ADRA1B |
| SOU-001 | for treatment of incontinence | ADRA1D |
| SOU-003 | for treatment of incontinence | AVPR2 |
| doxorubicin | antineoplastic agent | TOP2A |
| carbamazepine | for treatment of bipolar disorder | SCN5A |
| mesalamine | for treatment of ulcerative colitis | ALOX5 |
| mesalamine | for treatment of ulcerative colitis | CHUK |
| mesalamine | for treatment of ulcerative colitis | IKBKB |
| mesalamine | for treatment of ulcerative colitis | PPARG |
| mesalamine | for treatment of ulcerative colitis | PTGS1 |
| mesalamine | for treatment of ulcerative colitis | PTGS2 |
| allopurinol | antiuricemic agent | XDH |
| SPP676 | antihypertensive agent | REN |
| Resveratrol | antidiabetic, antineoplastic agent | PDE4B |
| Resveratrol | antidiabetic, antineoplastic agent | PDE4D |
| ganetespib | antineoplastic agent | HSP90AA1 |
| ganetespib | antineoplastic agent | HSP90AB1 |
| stannsoporfin | for prevention of hyperbilirubinemia | HMOX1 |
| stannsoporfin | for prevention of hyperbilirubinemia | HMOX2 |
| nateglinide | antidiabetic | ABCC8 |
| morphine | analgesic | OPRK1 |
| morphine | analgesic | OPRK1 |
| morphine | analgesic | OPRK1 |
| strontium ranelate | antiosteoporotic agent | CASR |
| STX107 | for treatment of Fragile X symptoms | GRM5 |
| sucralfate | antiulcer agent | PGA3 |
| sufentanil | analgesic | OPRD1 |
| sufentanil | analgesic | OPRK1 |
| sufentanil | analgesic | OPRM1 |
| sufentanil | analgesic | OPRD1 |
| sufentanil | analgesic | OPRK1 |
| sufentanil | analgesic | OPRM1 |
| sulfasalazine | antiinflammatory agent, DMARD | ACAT1 |
| sulfasalazine | antiinflammatory agent, DMARD | PPARG |
| sulfasalazine | antiinflammatory agent, DMARD | PTGS1 |
| sulfasalazine | antiinflammatory agent, DMARD | PTGS2 |
| sulodexide | for treatment of diabetic nephropathy | SERPINC1 |
| sulodexide | for treatment of diabetic nephropathy | SERPIND1 |
| Sumatriptan | antimigraine agent | HTR1A |
| Sumatriptan | antimigraine agent | HTR1B |
| Sumatriptan | antimigraine agent | HTR1D |
| Sumatriptan | antimigraine agent | HTR1F |
| Sumatriptan | antimigraine agent | HTR1A |
| Sumatriptan | antimigraine agent | HTR1B |
| Sumatriptan | antimigraine agent | HTR1D |
| Sumatriptan | antimigraine agent | HTR1F |
| Sumatriptan | antimigraine agent | HTR1A |
| Sumatriptan | antimigraine agent | HTR1B |
| Sumatriptan | antimigraine agent | HTR1D |
| Sumatriptan | antimigraine agent | HTR1F |
| surinabant | smoking-cessation agent | CNR1 |
| latanoprost | for treatment of glaucoma | PTGFR |
| sunitinib | antineoplastic agent | FLT1 |
| sunitinib | antineoplastic agent | FLT3 |
| sunitinib | antineoplastic agent | FLT4 |
| sunitinib | antineoplastic agent | KDR |
| sunitinib | antineoplastic agent | KIT |
| sunitinib | antineoplastic agent | PDGFRA |
| sunitinib | antineoplastic agent | PDGFRB |
| sunitinib | antineoplastic agent | RET |
| SUVN-502 | for treatment of Alzheimer's disease | HTR6 |
| SVT-40776 | for treatment of incontinence | CHRM3 |
| tozadenant | antiparkinson agent | ADORA2A |
| nitisinone | antiparkinson agent | HPD |
| T-5224 | antiinflammatory agent, DMARD | JUN |
| T-62 | analgesic | ADORA1 |
| tacrolimus | immunosuppressant | FKBP1A |
| tacrolimus | immunosuppressant | FKBP1A |
| TAFA-93 | immunosuppressant | FRAP1 |
| TAK-242 | for treatment of sepsis | TLR4 |
| dexlansoprazole | Proton pump inhibitor | ATP4A |
| TAK-442 | antithrombotic | F10 |
| Talabostat | for treatment of neutropenia | CSF3 |
| talampanel | antiparkinson agent, antineoplastic agent | GRIA1 |
| talampanel | antiparkinson agent, antineoplastic agent | GRIA2 |
| talampanel | antiparkinson agent, antineoplastic agent | GRIA3 |
| talampanel | antiparkinson agent, antineoplastic agent | GRIA4 |
| talarozole | antipsoriatic agent, for treatment of acne | CYP26A1 |
| talarozole | antipsoriatic agent, for treatment of acne | CYP26B1 |
| talarozole | antipsoriatic agent, for treatment of acne | CYP26C1 |
| talnetant | antipsychotic agent | TACR3 |
| talotrexin | antineoplastic agent | DHFR |
| Tamibarotene | antineoplastic agent | RARA |
| Tamibarotene | antineoplastic agent | RARB |
| tamsulosin | for treatment of urinary symptoms associated | ADRA1A |
| | with BPH | |
| tamsulosin | for treatment of urinary symptoms associated | ADRA1B |
| | with BPH | |
| tamsulosin | for treatment of urinary symptoms associated | ADRA1D |
| | with BPH | |
| tandutinib | antineoplastic agent | FLT3 |
| Tanespimycin | antineoplastic agent | HSP90AA1 |
| Tanespimycin | antineoplastic agent | HSP90AB1 |
| tapentadol | analgesic | OPRM1 |
| tapentadol | analgesic | SLC6A2 |
| tapentadol | analgesic, opioid | MOR |
| Taranabant | antiobesity agent, smoking-cessation agent | CNR1 |
| crlotinib | antincoplastic agent | EGFR |
| tariquidar | adjuvant to chemotherapy | ABCB1 |
| TAS-108 | antineoplastic agent | ESR1 |
| TAS-108 | antineoplastic agent | ESR2 |
| tasimelteon | hypnotic | MTNR1A |
| tasimelteon | hypnotic | MTNR1B |
| Tasocitinib | antiinflammatory agent, DMARD | JAK3 |
| tazarotene | antipsoriatic agent, for treatment of acne | RARA |
| tazarotene | antipsoriatic agent, for treatment of acne | RARB |
| tazarotene | antipsoriatic agent, for treatment of acne | RARG |
| tazarotene | antipsoriatic agent, for treatment of acne | RXRB |
| TBR-652 | antiviral agent, HIV | CCR5 |
| ispronicline | nootropic | CHRNA4 |
| ispronicline | nootropic | CHRNB2 |
| TC-2403-12 | for treatment of ulcerative colitis | CHRNA4 |
| TC-2403-12 | for treatment of ulcerative colitis | CHRNB2 |
| TC-2696 | analgesic | CHRNA4 |
| TC-2696 | analgesic | CHRNB2 |
| TC-5214 | antidepressant | CHRNA4 |
| TC-5214 | antidepressant | CHRNB2 |
| TC-5619 | neuroprotectant | CHRNA7 |
| TC-6499 | analgesic, neuropathic pain | CHRNA4 |
| TC-6499 | analgesic, neuropathic pain | CHRNB2 |
| TC-6987 | antiasthmatic agent, antidiabetic | CHRNA7 |
| TD-1211 | for treatment of opioid-induced gastrointestinal | OPRM1 |
| | side-effects | |
| tecadenoson | antiarrhytmic agent | ADORA1 |
| tecarfarin | antithrombotic | VKORC1 |
| tegaserod | motilitant | HTR4 |
| telatinib | antineoplastic agent | FLT1 |
| telatinib | antineoplastic agent | FLT4 |
| telatinib | antineoplastic agent | KDR |
| telatinib | antineoplastic agent | PDGFRA |
| telatinib | antineoplastic agent | PDGFRB |
| telmisartan | antihypertensive agent | AGTR1 |
| temsirolimus | antineoplastic agent | FRAP1 |
| terguride | for treatment of pulmonary arterial hypertension | HTR2A |
| terguride | for treatment of pulmonary arterial hypertension | HTR2B |
| teriflunomide | for treatment of multiple sclerosis | DHODH |
| terlipressin | for treatment of hepatorenal syndrome | AVPR1A |
| terlipressin | for treatment of hepatorenal syndrome | AVPR1B |
| terlipressin | for treatment of hepatorenal syndrome | AVPR2 |
| tesetaxel | antineoplastic agent | BCL2 |
| tesetaxel | antineoplastic agent | TUBB1 |
| tesmilifene | adjuvant to chemotherapy | ABCB1 |
| tesmilifene | adjuvant to chemotherapy | CYP3A4 |
| tesmilifene | adjuvant to chemotherapy | CYP3A5 |
| tesmilifene | adjuvant to chemotherapy | CYP3A7 |
| tesofensine | antiobesity agent | SLC6A2 |
| tesofensine | antiobesity agent | SLC6A4 |
| testosterone | hormone replacement, for treatment of female | AR |
| | sexual dysfunction | |
| testosterone | for treatment of female sexual dysfunction | AR |
| testosterone | hormone replacement | AR |
| testosterone | for treatment of female sexual dysfunction | AR |
| testosterone | hormone replacement | AR |
| testosterone | hormone replacement | AR |
| testosterone | for treatment of female sexual dysfunction | AR |
| tetrabenazine | for treatment of Huntington's disease | SLC18A2 |
| tetrodotoxin | analgesic | SCN10A |
| tetrodotoxin | analgesic | SCN11A |
| tetrodotoxin | analgesic | SCN1A |
| tetrodotoxin | analgesic | SCN2A |
| tetrodotoxin | analgesic | SCN3A |
| tetrodotoxin | analgesic | SCN4A |
| tetrodotoxin | analgesic | SCN5A |
| tetrodotoxin | analgesic | SCN8A |
| tetrodotoxin | analgesic | SCN9A |
| tezampanel | antimigraine agent, analgesic | GRIA1 |
| tezampanel | antimigraine agent, analgesic | GRIA2 |
| tezampanel | antimigraine agent, analgesic | GRIA3 |
| tezampanel | antimigraine agent, analgesic | GRIA4 |
| tezampanel | antimigraine agent, analgesic | GRIK1 |
| tezampanel | antimigraine agent, analgesic | GRIK2 |
| tezampanel | antimigraine agent, analgesic | GRIK3 |
| tezampanel | antimigraine agent, analgesic | GRIK4 |
| tezampanel | antimigraine agent, analgesic | GRIK5 |
| TG-0054 | adjuvant to stem cell transplantation | CXCR4 |
| TG02, SB1317 | antineoplastic agent | CDK2 |
| TG02, SB1317 | antineoplastic agent | ERK5 |
| TG02, SB1317 | antineoplastic agent | FLT3 |
| TG02, SB1317 | antineoplastic agent | JAK2 |
| TG101348 | antineoplastic agent | JAK2 |
| thalidomide | antineoplastic agent | FGFR2 |
| thalidomide | antineoplastic agent | NFKB1 |
| thalidomide | antineoplastic agent | PTGS2 |
| thalidomide | antineoplastic agent | TNF |
| sitaxsentan | for treatment of pulmonary arterial hypertension | EDNRA |
| ketoprofen | NSAID | PTGS1 |
| ketoprofen | NSAID | PTGS1 |
| ketoprofen | NSAID | PTGS2 |
| ketoprofen | NSAID | PTGS2 |
| pilocarpine | for treatment of incontinence | CHRM1 |
| pilocarpine | for treatment of incontinence | CHRM2 |
| pilocarpine | for treatment of incontinence | CHRM3 |
| tolterodine | for treatment of incontinence | CHRM1 |
| tolterodine | for treatment of incontinence | CHRM2 |
| toltcrodinc | for treatment of incontinence | CHRM3 |
| tolterodine | for treatment of incontinence | CHRM4 |
| tolterodine | for treatment of incontinence | CHRM5 |
| Ticagrelor | antithrombotic | P2RY12 |
| tideglusib | for treatment of Alzheimer's disease | GSK3A |
| tideglusib | for treatment of Alzheimer's disease | GSK3B |
| tilarginine | for treatment of cardiogenic shock | NOS2 |
| tiotropium | for treatment of cystic fibrosis, for treatment of | CHRM1 |
| | chronic obstructive pulmonary disorder (COPD) | |
| tiotropium | for treatment of cystic fibrosis, for treatment of | CHRM2 |
| | chronic obstructive pulmonary disorder (COPD) | |
| tiotropium | for treatment of cystic fibrosis, for treatment of | CHRM3 |
| | chronic obstructive pulmonary disorder (COPD) | |
| tipifarnib | antineoplastic agent | FNTA |
| tipifarnib | antineoplastic agent | FNTB |
| tizanidine | muscle relaxant | ADRA2A |
| tizanidine | muscle relaxant | ADRA2B |
| tizanidine | muscle relaxant | ADRA2C |
| canfosfamide | antineoplastic agent | GSTP1 |
| TLN-4601 | antineoplastic agent | TSPO |
| obinepitide | antiobesity agent | NPY2R |
| obinepitide | antiobesity agent | PPYR1 |
| TM30339 | antiobesity agent | PPYR1 |
| TM38837 | antiobesity agent | CNR1 |
| ondansetron | for treatment of obsessive compulsive disorder | HTR3A |
| | (OCD) | |
| galeterone | antineoplastic agent | AR |
| galeterone | antineoplastic agent | CYP17A1 |
| tolterodine | for treatment of incontinence | CHRM1 |
| tolterodine | for treatment of incontinence | CHRM2 |
| tolterodine | for treatment of incontinence | CHRM3 |
| tolterodine | for treatment of incontinence | CHRM4 |
| tolterodine | for treatment of incontinence | CHRM5 |
| tolvaptan | antihypertensive agent | AVPR2 |
| Tonabersat | antimigraine agent | HTR1D |
| alprostadil | for treatment of erectile dysfunction, for | PTGER1 |
| | treatment of sexual dysfunction in women | |
| alprostadil | for treatment of erectile dysfunction, for | PTGER2 |
| | treatment of sexual dysfunction in women | |
| menadione | for reducing EGFR-inhibitor-induced | GGCX |
| | dermatological side effects | |
| menadione | for reducing EGFR-inhibitor-induced | VKORC1 |
| | dermatological side effects | |
| menadione | for reducing EGFR-inhibitor-induced | VKORC1L1 |
| | dermatological side effects | |
| testosterone | hormone replacement | AR |
| topiramate | anticonvulsant | CA2 |
| topiramate | anticonvulsant | CA4 |
| topiramate | anticonvulsant | GABRA1 |
| topiramate | anticonvulsant | GRIK1 |
| topiramate | anticonvulsant | SCN1A |
| topiramate | anticonvulsant, antimigraine agent | CA2 |
| topiramate | anticonvulsant, antimigraine agent | CA4 |
| topiramate | anticonvulsant, antimigraine agent | GABRA1 |
| topiramate | anticonvulsant, antimigraine agent | GRIK1 |
| topiramate | anticonvulsant, antimigraine agent | SCN1A |
| topotecan | antineoplastic agent | TOP1 |
| Torcetrapib | antidyslipidaemic agent | CETP |
| morphine | analgesic | OPRD1 |
| morphine | analgesic | OPRK1 |
| morphine | analgesic | OPRM1 |
| bosentan | for treatment of pulmonary arterial hypertension | EDNRA |
| bosentan | for treatment of pulmonary arterial hypertension | EDNRB |
| tramadol | analgesic | HTR2C |
| tramadol | analgesic | OPRK1 |
| tramadol | analgesic | OPRM1 |
| tramadol | analgesic | OPRM1 |
| tramadol | analgesic | SLC6A2 |
| tramadol | analgesic | SLC6A2 |
| tramadol | analgesic | SLC6A4 |
| tramadol | analgesic | SLC6A4 |
| tramadol | analgesic | HTR2C |
| tramadol | analgesic | OPRK1 |
| tramadol | analgesic | OPRM1 |
| tramadol | analgesic | OPRM1 |
| tramadol | analgesic | SLC6A2 |
| tramadol | analgesic | SLC6A2 |
| tramadol | analgesic | SLC6A4 |
| tramadol | analgesic | SLC6A4 |
| tramadol | analgesic | HTR2C |
| tramadol | analgesic | OPRK1 |
| tramadol | analgesic | OPRM1 |
| tramadol | analgesic | OPRM1 |
| tramadol | analgesic | SLC6A2 |
| tramadol | analgesic | SLC6A2 |
| tramadol | analgesic | SLC6A4 |
| tramadol | analgesic | SLC6A4 |
| homotaurine | for treatment of Alzheimer's disease | APP |
| trandolapril | antihypertensive agent | ACE |
| tranexamic acid | antimenorrhagic agent | PLAT |
| capsaicin | analgesic | TRPV1 |
| diclofenac | NSAID | PTGS1 |
| diclofenac | NSAID | PTGS2 |
| estradiol | hormone replacement | ESR1 |
| estradiol | hormone replacement | ESR2 |
| granisetron | antiemetic | HTR3A |
| lidocaine | anestethic | SCN10A |
| lidocaine | anestethic | SCN5A |
| lidocaine | anestethic | SCN9A |
| epinephrine | anestethic | ADRA1A |
| epinephrine | anestethic | ADRA1B |
| epinephrine | anestethic | ADRA1D |
| epinephrine | anestethic | ADRA2A |
| epinephrine | anestethic | ADRA2B |
| epinephrine | anestethic | ADRB1 |
| epinephrine | anestethic | ADRB2 |
| lidocaine | anestethic | SCN10A |
| lidocaine | anestethic | SCN5A |
| lidocaine | anestethic | SCN9A |
| oxybutynin | for treatment of incontinence | CHRM1 |
| oxybutynin | for treatment of incontinence | CHRM2 |
| oxybutynin | for treatment of incontinence | CHRM3 |
| oxycodone | analgesic | OPRD1 |
| oxycodone | analgesic | OPRK1 |
| oxycodone | analgesic | OPRM1 |
| fentanyl | analgesic | OPRD1 |
| fentanyl | analgesic | OPRM1 |
| timolol | for treatment of glaucoma | ADRB1 |
| timolol | for treatment of glaucoma | ADRB2 |
| travoprost | for treatment of glaucoma | PTGFR |
| trazodone | antidepressant | HTR1A |
| trazodone | antidepressant | HTR2A |
| trazodone | antidepressant | HTR2C |
| trazodone | antidepressant | SLC6A4 |
| trelanserin | for treatment of intermittent claudication | HTR1B |
| trelanserin | for treatment of intermittent claudication | HTR2A |
| tretinoin | for treatment of acne | RARG |
| tretinoin | for treatment of acne | RXRB |
| tretinoin | for treatment of acne | RXRG |
| triamcinolone | for treatment of diabetic macular edema | NR3C1 |
| Triapine | antineoplastic agent | RRM2 |
| amlodipine | antihypertensive agent | CACNA1C |
| amlodipine | antihypertensive agent | CACNA1D |
| amlodipine | antihypertensive agent | CACNA1S |
| amlodipine | antihypertensive agent | CACNA2D1 |
| amlodipine | antihypertensive agent | CACNB2 |
| hydrochlorothiazide | antihypertensive agent | SLC12A3 |
| olmesartan | antihypertensive agent | AGTR1 |
| triciribine | antineoplastic agent | AKT1 |
| triciribine | antineoplastic agent | AKT2 |
| triciribine | antineoplastic agent | AKT3 |
| HE3286 | antiinflammatory agent, DMARD | NR3C1 |
| trodusquemine | antiobesity agent | PTPN1 |
| trospium | for treatment of incontinence | CHRM1 |
| TTP889 | anticoagulant | F9 |
| lapatinib | antineoplastic agent | EGFR |
| lapatinib | antineoplastic agent | ERBB2 |
| TZP-101 | for treatment of gastroparesis | GHSR |
| TZP-102 | for treatment of gastroparesis | GHSR |
| heparin | for treatment of pelvic pain of bladder origin and | F10 |
| | interstitital cystitis | |
| heparin | for treatment of pelvic pain of bladder origin and | SERPINC1 |
| | interstitital cystitis | |
| lidocaine | for treatment of pelvic pain of bladder origin and | SCN10A |
| | interstitital cystitis | |
| lidocaine | for treatment of pelvic pain of bladder origin and | SCN5A |
| | interstitital cystitis | |
| lidocaine | for treatment of pelvic pain of bladder origin and | SCN9A |
| | interstitital cystitis | |
| udenafil | for treatment of erectile dysfunction | PDE5A |
| tegafur | antineoplastic agent | TYMS |
| Ulipristal | contraceptive | PGR |
| heparin | antithrombotic | F10 |
| heparin | antithrombotic | SERPINC1 |
| ursodeoxycholic acid | for prevention of recurrence of colorectal polyps | AKR1C2 |
| topiramate | anticonvulsant | CA2 |
| topiramate | anticonvulsant | CA4 |
| topiramate | anticonvulsant | GABRA1 |
| topiramate | anticonvulsant | GRIK1 |
| topiramate | anticonvulsant | SCN1A |
| buprenorphine | antidepressant, analgesic, for treatment of opioid | OPRD1 |
| | addiction | |
| buprenorphine | antidepressant, analgesic, for treatment of opioid | OPRK1 |
| | addiction | |
| buprenorphine | antidepressant, analgesic, for treatment of opioid | OPRM1 |
| | addiction | |
| carbidopa | antiparkinson agent | DDC |
| melevodopa | antiparkinson agent | DRD1 |
| melevodopa | antiparkinson agent | DRD2 |
| melevodopa | antiparkinson agent | DRD3 |
| melevodopa | antiparkinson agent | DRD4 |
| melevodopa | antiparkinson agent | DRD5 |
| V158866 | analgesic | FAAH |
| V24343 | antiobesity agent | CNR1 |
| V3381 | analgesic, neuropathic pain | GRIN1 |
| V3381 | analgesic, neuropathic pain | GRIN2A |
| V3381 | analgesic, neuropathic pain | GRIN2B |
| V3381 | analgesic, neuropathic pain | GRIN2C |
| V3381 | analgesic, neuropathic pain | GRIN2D |
| V3381 | analgesic, neuropathic pain | GRIN3A |
| V3381 | analgesic, neuropathic pain | GRIN3B |
| V3381 | analgesic, neuropathic pain | MAOA |
| V3381 | analgesic, neuropathic pain | MAOB |
| VA106483 | for treatment of BPH-related urinary symptoms | AVPR2 |
| VA111913 | for treatment of dysmenorrhea | AVPR1A |
| VA111913 | for treatment of dysmenorrhea | AVPR1B |
| VA111913 | for treatment of dysmenorrhea | AVPR2 |
| Vadimezan | antineoplastic agent | HIPK2 |
| Vadimezan | antineoplastic agent | KDR |
| Vadimezan | antineoplastic agent | PIM3 |
| valproic acid | anticonvulsant | ABAT |
| valproic acid | anticonvulsant | ACADSB |
| valproic acid | anticonvulsant | HDAC9 |
| valproic acid | for treatment of basal cell carcinoma | ABAT |
| valproic acid | for treatment of basal cell carcinoma | ACADSB |
| valproic acid | for treatment of basal cell carcinoma | HDAC9 |
| valsartan | antihypertensive agent | AGTR1 |
| vapitadine | antiallergy agent | HRH1 |
| vapreotide | for treatment of liver cirrhosis-related variceal | SSTR2 |
| | bleeding | |
| vapreotide | for treatment of liver cirrhosis-related variceal | SSTR5 |
| | bleeding | |
| vardenafil | for treatment of erectile dysfunction | PDE5A |
| varenicline | smoking-cessation agent | CHRNA3 |
| varenicline | smoking-cessation agent | CHRNA4 |
| varenicline | smoking-cessation agent | CHRNA7 |
| varenicline | smoking-cessation agent | CHRNB2 |
| varenicline | smoking-cessation agent | CHRNB4 |
| varespladib | antiinflammatory agent | PLA2G10 |
| varespladib | antiinflammatory agent | PLA2G2A |
| varcspladib | antiinflammatory agent | PLA2G5 |
| varespladib | antiinflammatory agent | PLA2G10 |
| varespladib | antiinflammatory agent | PLA2G2A |
| varespladib | antiinflammatory agent | PLA2G5 |
| ethinyl estradiol | contraceptive | ESR1 |
| norethindrone | contraceptive | PGR |
| Vatalanib | antineoplastic agent | FLT1 |
| Vatalanib | antineoplastic agent | FLT4 |
| Vatalanib | antineoplastic agent | KDR |
| Vatalanib | antineoplastic agent | KIT |
| Vatalanib | antineoplastic agent | PDGFRA |
| Vatalanib | antineoplastic agent | PDGFRB |
| Vatalanib | antineoplastic agent | FLT1 |
| Vatalanib | antineoplastic agent | FLT4 |
| Vatalanib | antineoplastic agent | KDR |
| Vatalanib | antineoplastic agent | KIT |
| Vatalanib | antineoplastic agent | PDGFRA |
| Vatalanib | antineoplastic agent | PDGFRB |
| VEL-0230 | antirheumatic agent | CTSK |
| bortezomib | antineoplastic agent | PSMB1 |
| bortezomib | antineoplastic agent | PSMB2 |
| bortezomib | antineoplastic agent | PSMB5 |
| bortezomib | antineoplastic agent | PSMD1 |
| bortezomib | antineoplastic agent | PSMD2 |
| bupropion | antidepressant, appetite suppressant, smoking- | SLC6A2 |
| | cessation agent | |
| bupropion | antidepressant, appetite suppressant, smoking- | SLC6A3 |
| | cessation agent | |
| velneperit | antiobesity agent | NPY5R |
| velusetrag | motilitant | HTR4 |
| fluticasone furoate | antiinflammatory agent, glucocorticoid | NR3C1 |
| verapamil | antihypertensive agent | CACNA1C |
| verapamil | antihypertensive agent | CACNA1D |
| verapamil | antihypertensive agent | CACNA1F |
| verapamil | antihypertensive agent | CACNA1S |
| verapamil | antihypertensive agent | CACNB1 |
| verapamil | antihypertensive agent | CACNB2 |
| vorapamil | antihypertensive agent | CACNB3 |
| verapamil | antihypertensive agent | CACNB4 |
| vestipitant | for treatment of tinnitus, hypnotic | TACR1 |
| VGX-1027 | antiinflammatory agent, DMARD | unknown |
| VIA-2291 | antiatherosclerotic agent | ALOX5 |
| VIA-3196 | antidyslipidaemic agent | THRB |
| Calcitonin | antiosteoporotic agent | CALCR |
| methotrexate | DMARD | DHFR |
| vicriviroc | antiviral agent, HIV | CCR5 |
| vidofludimus | antiinflammatory agent, DMARD | DHODH |
| vidofludimus | antiinflammatory agent, DMARD | IL17A |
| vidofludimus | antiinflammatory agent, DMARD | IL17B |
| vidofludimus | antiinflammatory agent, DMARD | IL17C |
| vidofludimus | antiinflammatory agent, DMARD | IL17D |
| vidofludimus | antiinflammatory agent, DMARD | IL17E |
| Vigabatrin | for treatment of addiction | ABAT |
| Vigabatrin | for treatment of addiction | GABBR1 |
| vilazodone | antidepressant | HTR1A |
| vildagliptin | antidiabetic | DPP4 |
| vincristine | antineoplastic agent | TUBA4A |
| vincristine | antineoplastic agent | TUBB |
| vinorelbine | antineoplastic agent | TUBB |
| BIIB014 | antiparkinson agent | ADORA2A |
| Virulizin | antineoplastic agent | IL12A |
| Virulizin | antineoplastic agent | IL12B |
| naltrexone | for treatment of substance abuse | OPRD1 |
| naltrexone | for treatment of substance abuse | OPRK1 |
| naltrexone | for treatment of substance abuse | OPRM1 |
| Voclosporin | antiinflammatory | PPIA |
| | agent, DMARD, immunosuppressant | |
| Voclosporin | antiinflammatory | PPP3CA |
| | agent, DMARD, immunosuppressant | |
| Voclosporin | antiinflammatory | PPP3CB |
| | agent, DMARD, immunosuppressant | |
| Voclosporin | antiinflammatory | PPP3CC |
| | agent, DMARD, immunosuppressant | |
| vofopitant | for treatment of post-traumatic stress | TACR1 |
| | disorder, hypnotic | |
| voglibose | antidiabetic | MGAM |
| volinanserin | hypnotic | HTR2A |
| vorapaxar | cardiovascular agent | F2R |
| vorapaxar | cardiovascular agent | F2RL2 |
| vorapaxar | cardiovascular agent | F2RL3 |
| Voreloxin | antineoplastic agent | TOP2A |
| Voreloxin | antineoplastic agent | TOP2B |
| Histrelin | antineoplastic agent | GNRHR |
| Histrelin | antineoplastic agent | GNRHR2 |
| TRPV1 antagonist | analgesic | TRPV1 |
| VR-147 | antimigraine agent | HTR1B |
| VR-147 | antimigraine agent | HTR1D |
| heparin | for treatment of cystic fibrosis | F10 |
| heparin | for treatment of cystic fibrosis | SERPINC1 |
| etodolac | for treatment of cancer cachexia | PTGS1 |
| etodolac | NSAID | PTGS2 |
| propranolol | for treatment of cancer cachexia | ADRB1 |
| VTP-27999 | antihypertensive agent | REN |
| VTX-1463 | antiallergy agent | TLR8 |
| VTX-2337 | antineoplastic agent | TLR8 |
| VX-509 | antiinflammatory agent, DMARD | JAK3 |
| WX-554 | antineoplastic agent | MAP2K1 |
| WX-554 | antineoplastic agent | MAP2K2 |
| WX-554 | antineoplastic agent | MAP2K3 |
| WX-554 | antineoplastic agent | MAP2K4 |
| WX-554 | antineoplastic agent | MAP2K5 |
| WX-554 | antineoplastic agent | MAP2K6 |
| WX-554 | antineoplastic agent | MAP2K7 |
| tozasertib | antineoplastic agent | AURKA |
| tozasertib | antineoplastic agent | AURKB |
| tozasertib | antineoplastic agent | AURKC |
| VX-702 | antiinflammatory agent, cardiovascular agent | MAPK11 |
| VX-702 | antiinflammatory agent, cardiovascular agent | MAPK12 |
| VX-702 | antiinflammatory agent, cardiovascular agent | MAPK13 |
| VX-702 | antiinflammatory agent, cardiovascular agent | MAPK14 |
| VX-765 | antipsoriatic agent, anticonvulsant | CASP1 |
| ivacaftor | for treatment of cystic fibrosis | CFTR |
| VX-809 | for treatment of cystic fibrosis | CFTR |
| ivacaftor | for treatment of cystic fibrosis | CFTR |
| VX-809 | for treatment of cystic fibrosis | CFTR |
| WX-UK1 | antineoplastic agent | PLAU |
| emzetibe | antidyslipidaemic agent | NPC1L1 |
| emzetibe | antidyslipidaemic agent | SOAT1 |
| simvastatin | antidyslipidaemic agent | HMGCR |
| NRP104 | for treatment of ADHD | ADRA1B |
| NRP104 | for treatment of ADHD | SLC18A2 |
| NRP104 | for treatment of ADHD | SLC6A3 |
| xaliproden | neuroprotectant | HTR1A |
| XL019 | antineoplastic agent | JAK2 |
| cabozantinib | antineoplastic agent | KDR |
| cabozantinib | antineoplastic agent | MET |
| XL228 | antineoplastic agent | ABL1 |
| XL228 | antineoplastic agent | AURKA |
| XL228 | antineoplastic agent | IGF1R |
| XL228 | antineoplastic agent | SRC |
| XL281 | antineoplastic agent | ARAF |
| XL281 | antineoplastic agent | BRAF |
| XL281 | antineoplastic agent | RAF1 |
| XL418 | antineoplastic agent | AKT1 |
| XL418 | antineoplastic agent | AKT2 |
| XL418 | antineoplastic agent | AKT3 |
| XL418 | antineoplastic agent | RPS6KB1 |
| XL647 | antineoplastic agent | EGFR |
| XL647 | antineoplastic agent | EPHB4 |
| XL647 | antineoplastic agent | ERBB2 |
| XL647 | antineoplastic agent | FLT1 |
| XL647 | antineoplastic agent | FLT4 |
| XL647 | antineoplastic agent | KDR |
| XL765 | antineoplastic agent | MTOR |
| XL765 | antineoplastic agent | PIK3CA |
| XL765 | antineoplastic agent | PIK3CD |
| XL765 | antineoplastic agent | PIK3CG |
| XL820 | antineoplastic agent | FLT1 |
| XL820 | antincoplastic agent | FLT4 |
| XL820 | antineoplastic agent | KDR |
| XL820 | antineoplastic agent | KIT |
| XL820 | antineoplastic agent | PDGFRA |
| XL820 | antineoplastic agent | PDGFRB |
| XL844 | antineoplastic agent | CHEK1 |
| XL844 | antineoplastic agent | CHEK2 |
| XL880 | antineoplastic agent | KDR |
| XL880 | antineoplastic agent | MET |
| XL888 | antineoplastic agent | HSP90AA1 |
| XL888 | antineoplastic agent | HSP90AB1 |
| XL999 | antineoplastic agent | AXL |
| XL999 | antineoplastic agent | FGFR1 |
| XL999 | antineoplastic agent | FLT1 |
| XL999 | antineoplastic agent | FLT3 |
| XL999 | antineoplastic agent | FLT4 |
| XL999 | antineoplastic agent | KDR |
| XL999 | antineoplastic agent | KIT |
| XL999 | antineoplastic agent | PDGFRB |
| camptothecin | antineoplastic agent | TOP1 |
| XMT-1107 | antineoplastic agent | METAP2 |
| tranexamic acid | for treatment of menorrhagia | PLG |
| XP13512 | for treatment of restless legs syndrome | CACNA1B |
| XP13512 | for treatment of restless legs syndrome | CACNA2D1 |
| XP13512 | for treatment of restless legs syndrome | CACNA2D2 |
| R-baclofen | for treatment of gastrointestinal reflux disease | GABBR1 |
| R-baclofen | for treatment of gastrointestinal reflux disease | GABBR2 |
| XP21279 | antiparkinson agent | DRD1 |
| XP21279 | antiparkinson agent | DRD2 |
| XP21279 | antiparkinson agent | DRD3 |
| XP21279 | antiparkinson agent | DRD4 |
| XP21279 | antiparkinson agent | DRD5 |
| gantofiban | antithrombotic, antiatherosclerotic agent | ITGA2B |
| gantofiban | antithrombotic, antiatherosclerotic agent | ITGB3 |
| finasteride | antineoplastic agent | AKR1D1 |
| finasteride | antineoplastic agent | SRD5A1 |
| finasteride | antincoplastic agent | SRD5A2 |
| YM-178 | for treatment of overactive bladder | ADRB3 |
| YM-598 | antineoplastic agent | EDNRA |
| vandetanib | antineoplastic agent | EGFR |
| vandetanib | antineoplastic agent | FLT1 |
| vandetanib | antineoplastic agent | FLT4 |
| vandetanib | antineoplastic agent | KDR |
| vandetanib | antineoplastic agent | RET |
| zafirlukast | antiasthmatic agent | CYSLTR1 |
| zaleplon | hypnotic | GABRA1 |
| zaleplon | hypnotic | TSPO |
| ranitidine | antiulcer agent | HRH2 |
| beloranib | antiobesity agent | METAP2 |
| zibotentan | antineoplastic agent | EDNRA |
| ziconotide | analgesic | CACNA1B |
| ziprasidone | antipsychotic agent | DRD2 |
| ziprasidone | antipsychotic agent | HTR2A |
| ondansetron | antiemetic | HTR3A |
| zoledronate | antiosteoporotic agent | FDPS |
| zoledronate | antiosteoporotic agent | GGPS1 |
| zolmitriptan | antimigraine agent | HTR1A |
| zolmitriptan | antimigraine agent | HTR1B |
| zolmitriptan | antimigraine agent | HTR1D |
| zolmitriptan | antimigraine agent | HTR1F |
| sertraline | antidepressant, for treatment of obsessive | SLC6A3 |
| | compulsive disorder (OCD) | |
| sertraline | antidepressant, for treatment of obsessive | SLC6A4 |
| | compulsive disorder (OCD) | |
| zolpidem | hypnotic | GABRA1 |
| zonisamide | anticonvulsant | CACNA1G |
| zonisamide | anticonvulsant | CACNA1H |
| zonisamide | anticonvulsant | CACNA1I |
| zonisamide | anticonvulsant | SCN11A |
| zonisamide | anticonvulsant | SCN1A |
| zonisamide | anticonvulsant | SCN1B |
| zonisamidc | anticonvulsant | SCN2A |
| zonisamide | anticonvulsant | SCN2B |
| zonisamide | anticonvulsant | SCN3A |
| zonisamide | anticonvulsant | SCN3B |
| zonisamide | anticonvulsant | SCN4A |
| zonisamide | anticonvulsant | SCN4B |
| zonisamide | anticonvulsant | SCN5A |
| zonisamide | anticonvulsant | SCN9A |
| zosuquidar | adjuvant to chemotherapy | ABCB1 |
| zucapsaicin | analgesic | TRPV1 |
| hydrocodone | analgesic | OPRD1 |
| hydrocodone | analgesic | OPRM1 |
| zileuton | antiinflammatory agent | ALOX5 |
| ASP015K | for treatment of rheumatoid arthritis | JAK1 |
| ASP015K | for treatment of rheumatoid arthritis | JAK3 |
| CHF 6001 | antiasthmatic; for treatment of chronic | PDE4A |
| | obstructive pulmonary disease | |
| CHF 6001 | antiasthmatic; for treatment of chronic | PDE4B |
| | obstructive pulmonary disease | |
| CUDC-427 | antineoplastic agent | XIAP |
| ARQ 087 | antineoplastic agent | FGFR1 |
| ARQ 087 | antineoplastic agent | FGFR2 |
| ARQ 087 | antineoplastic agent | FGFR3 |
| deuterated dextromethorphan | for treatment of neurologic and psychiatric | GRIN3A |
| | disorders | |
| deuterated dextromethorphan | for treatment of neurologic and psychiatric | OPRS1 |
| | disorders | |
| olanzapine | antipsychotic agent | ADRA1A |
| olanzapine | antipsychotic agent | ADRA1B |
| olanzapine | antipsychotic agent | ADRA2A |
| olanzapine | antipsychotic agent | ADRA2B |
| olanzapine | antipsychotic agent | ADRA2C |
| olanzapine | antipsychotic agent | CHRM1 |
| olanzapine | antipsychotic agent | CHRM2 |
| olanzapine | antipsychotic agent | CHRM3 |
| olanzapine | antipsychotic agent | CHRM4 |
| olanzapine | antipsychotic agent | CHRM5 |
| olanzapine | antipsychotic agent | DRD1 |
| olanzapine | antipsychotic agent | DRD2 |
| olanzapine | antipsychotic agent | DRD3 |
| olanzapine | antipsychotic agent | DRD4 |
| olanzapine | antipsychotic agent | DRD5 |
| olanzapine | antipsychotic agent | HRH1 |
| olanzapine | antipsychotic agent | HTR1A |
| olanzapine | antipsychotic agent | HTR1B |
| olanzapine | antipsychotic agent | HTR1D |
| olanzapine | antipsychotic agent | HTR1E |
| olanzapine | antipsychotic agent | HTR2A |
| olanzapine | antipsychotic agent | HTR2C |
| olanzapine | antipsychotic agent | HTR3A |
| olanzapinc | antipsychotic agent | HTR6 |
| olanzapine | antipsychotic agent | HTR7 |
| samidoprhan | for treatment of addiction | MOR |
| Ethyl eicosapentaenoic acid | for treatment of cardiovascular disorders | PPARD |
| Ethyl eicosapentaenoic acid | for treatment of cardiovascular disorders | PPARG |
| Ethyl eicosapentaenoic acid | for treatment of cardiovascular disorders | PTGS1 |
| Ethyl eicosapentaenoic acid | for treatment of cardiovascular disorders | PTGS2 |
| BCX4161 | for treatment of hereditary angioedema | KLKB1 |
| ACEBUTOLOL | Antihypertensive Agents | ADRB1 |
| ACENOCOUMAROL | Anticoagulants | VKORC1 |
| ACEPROMETAZINE | Hypnotics and Sedatives | HRH1 |
| ACETAZOLAMIDE | Anticonvulsants; Diuretics; antiglaucomic agent | CA1 |
| ACETAZOLAMIDE | Anticonvulsants; Diuretics; antiglaucomic agent | CA12 |
| ACETAZOLAMIDE | Anticonvulsants; Diuretics; antiglaucomic agent | CA2 |
| ACETOHEXAMIDE | Hypoglycemic Agents | KCNJ1 |
| ACETOPHENAZINE | Antipsychotic Agents | DRD1 |
| ACETOPHENAZINE | Antipsychotic Agents | DRD2 |
| ACETYLDIGITOXIN | Anti-Arrhythmia Agents | ATP1A1 |
| ACITRETIN | Keratolytic Agents | RARA |
| ADAPALENE | Dermatologic Agents | RARA |
| ADAPALENE | Dermatologic Agents | RARB |
| ADAPALENE | Dermatologic Agents | RARG |
| ADAPALENE | Dermatologic Agents | RXRA |
| ADAPALENE | Dermatologic Agents | RXRB |
| ADAPALENE | Dermatologic Agents | RXRG |
| ADINAZOLAM | Anti-anxicty Agents; anticonvulsant | GABRA1 |
| ADINAZOLAM | Anti-anxiety Agents; anticonvulsant | GABRA2 |
| ADINAZOLAM | Anti-anxiety Agents; anticonvulsant | GABRA3 |
| ADINAZOLAM | Anti-anxiety Agents; anticonvulsant | GABRA5 |
| ADINAZOLAM | Anti-anxiety Agents; anticonvulsant | GABRB1 |
| ADINAZOLAM | Anti-anxiety Agents; anticonvulsant | GABRB2 |
| ADINAZOLAM | Anti-anxiety Agents; anticonvulsant | GABRB3 |
| ADINAZOLAM | Anti-anxiety Agents; anticonvulsant | GABRD |
| ADINAZOLAM | Anti-anxiety Agents; anticonvulsant | GABRE |
| ADINAZOLAM | Anti-anxiety Agents; anticonvulsant | GABRG1 |
| ADINAZOLAM | Anti-anxiety Agents; anticonvulsant | GABRG2 |
| ADINAZOLAM | Anti-anxiety Agents; anticonvulsant | GABRG3 |
| ADINAZOLAM | Anti-anxiety Agents; anticonvulsant | GABRP |
| ADINAZOLAM | Anti-anxiety Agents; anticonvulsant | GABRR1 |
| ADINAZOLAM | Anti-anxiety Agents; anticonvulsant | GABRR2 |
| ADINAZOLAM | Anti-anxiety Agents; anticonvulsant | GABRR3 |
| ALCAFTADINE | Anti-Allergic Agents | HRH1 |
| ALCLOMETASONE | Anti-Inflammatory Agents; Anti-pruritics; | NR3C1 |
| | Corticosteroids, topical | |
| ALENDRONATE | Bisphosphonates | FDPS |
| ALFENTANIL | Analgesics, Opioid | OPRM1 |
| Alitretionine | Antineoplastic Agents | RARA |
| Alitretionine | Antineoplastic Agents | RARB |
| Alitretionine | Antineoplastic Agents | RARG |
| Alitretionine | Antineoplastic Agents | RXRA |
| Alitretionine | Antineoplastic Agents | RXRB |
| Alitretionine | Antineoplastic Agents | RXRG |
| ALMITRINE | Respiratory Stimulant Agents | ATP1A1 |
| ALPRENOLOL | Anti-Arrhythmia Agents; Antihypertensive | ADRB1 |
| | Agents | |
| ALPRENOLOL | Anti-Arrhythmia Agents; Antihypertensive | ADRB2 |
| | Agents | |
| ALSEROXYLON | Antipsychotic Agents; Antihypertensive Agents | SLC18A2 |
| ALVIMOPAN | Opiate Antagonists | OPRM1 |
| AMBENONIUM | Antimyasthenics | ACHE |
| AMCINONIDE | Anti-Inflammatory Agents; Anti-pruritics; | NR3C1 |
| | Corticosteroids, topical | |
| AMINOCAPROIC ACID | Antifibrinolytic Agents | PLG |
| AMINOGLUTETHIMIDE | Antineoplastic agents | CYP19A1 |
| AMRINONE | Cardiotonic Agents; Phosphodiesterase | PDE3A |
| | Inhibitors | |
| AMRINONE | Cardiotonic Agents; Phosphodiesterase | PDE4B |
| | Inhibitors | |
| ANILERIDINE | Analgesics; Narcotics | OPRM1 |
| ANISINDIONE | Anticoagulants | GGCX |
| ANISOTROPINE | Antispasmodics; Anti-ulcer Agents | CHRM1 |
| METHYLBROMIDE | | |
| ANISOTROPINE | Antispasmodics; Anti-ulcer Agents | CHRM2 |
| METHYLBROMIDE | | |
| ANISOTROPINE | Antispasmodics; Anti-ulcer Agents | CHRM3 |
| METHYLBROMIDE | | |
| APRACLONIDINE | Antiglaucomic Agents | ADRA2A |
| APRINDINE | Anti-Arrhythmia Agents | SCN5A |
| ARBUTAMINE | Cardiotonic Agents | ADRB1 |
| ARDEPARIN | Anticoagulants | SERPINC1 |
| ARDEPARIN | Anticoagulants | SERPIND1 |
| ARFORMOTEROL | Bronchodilator Agents | ADRB2 |
| ASTEMIZOLE | Anti-Allergic Agents | HRH1 |
| ATENOLOL | Anti-Arrhythmia Agents; Antihypertensive | ADRB1 |
| | Agents | |
| ATRACURIUM | Muscle Relaxants | CHRNA2 |
| AURANOFIN | Antirheumatic Agents | IKBKB |
| AZATADINE | Anti-Allergic Agents | HRH1 |
| BENDROFLUMETHIAZIDE | Antihypertensive Agents; Diuretics | SLC12A3 |
| BENTIROMIDE | Diagnostic Agents | HPN |
| BENTIROMIDE | Diagnostic Agents | HPN |
| BENZOCAINE | Anesthetics, Local | SCN10A |
| BENZONATATE | Antitussive Agents | SCN5A |
| BENZPHETAMINE | Central Nervous System Stimulants | ADRA1A |
| BENZPHETAMINE | Central Nervous System Stimulants | ADRA2A |
| BENZQUINAMIDE | Antiemetics; Antipsychotic Agents | CHRM1 |
| BENZQUINAMIDE | Antiemetics; Antipsychotic Agents | CHRM2 |
| BENZQUINAMIDE | Antiemetics; Antipsychotic Agents | CHRM3 |
| BENZQUINAMIDE | Antiemetics; Antipsychotic Agents | CHRM4 |
| BENZQUINAMIDE | Antiemetics; Antipsychotic Agents | CHRM5 |
| BENZQUINAMIDE | Antiemetics; Antipsychotic Agents | HRH1 |
| BENZTHIAZIDE | Antihypertensive Agents; Diuretics | SLC12A3 |
| BENZTROPINE | Antiparkinson Agents | CHRM1 |
| BENZTROPINE | Antiparkinson Agents | SLC6A3 |
| BENZYLPENICILLOYL | Diagnostic Agents | FCER1A |
| POLYLYSINE | | |
| BENZYLPENICILLOYL | Diagnostic Agents | FCER1G |
| POLYLYSINE | | |
| BEPRIDIL | Anti-Arrhythmia Agents; Antihypertensive | ATP1A1 |
| | Agents | |
| BEPRIDIL | Anti-Arrhythmia Agents; Antihypertensive | CACNA1A |
| | Agents | |
| BEPRIDIL | Anti-Arrhythmia Agents; Antihypertensive | KCNQ1 |
| | Agents | |
| BEPRIDIL | Anti-Arrhythmia Agents; Antihypertensive | SCN5A |
| | Agents | |
| BEPRIDIL | Anti-Arrhythmia Agents; Antihypertensive | TNNC1 |
| | Agents | |
| BETAXOLOL | Antihypertensive Agents | ADRB1 |
| BETAZOLE | Diagnostic Agents | HRH2 |
| BETHANECHOL | Parasympathomimetics | CHRM1 |
| BETHANIDINE | Antihypertensive Agents | ADRA1A |
| BETHANIDINE | Antihypertensive Agents | ADRA1B |
| BETHANIDINE | Antihypertensive Agents | ADRA1D |
| BETHANIDINE | Antihypertensive Agents | ADRA2A |
| BETHANIDINE | Antihypertensive Agents | ADRA2B |
| BETHANIDINE | Antihypertensive Agents | ADRA2C |
| BETHANIDINE | Antihypertensive Agents | SLC6A2 |
| BEVANTOLOL | Antihypertensive Agents | ADRB1 |
| BIPERIDEN | Antidyskinetics | CHRM1 |
| BIPERIDEN | Antidyskinetics | CHRNA2 |
| BISOPROLOL | Antihypertensive Agents | ADRB1 |
| BRINZOLAMIDE | Antiglaucomic Agents | CA2 |
| BROMAZEPAM | Hypnotics and Sedatives | GABRA1 |
| BROMAZEPAM | Hypnotics and Sedatives | GABRA2 |
| BROMAZEPAM | Hypnotics and Sedatives | GABRA3 |
| BROMAZEPAM | Hypnotics and Sedatives | GABRA4 |
| BROMAZEPAM | Hypnotics and Sedatives | GABRA5 |
| BROMAZEPAM | Hypnotics and Sedatives | GABRA6 |
| BROMAZEPAM | Hypnotics and Sedatives | GABRB1 |
| BROMAZEPAM | Hypnotics and Sedatives | GABRB2 |
| BROMAZEPAM | Hypnotics and Sedatives | GABRB3 |
| BROMAZEPAM | Hypnotics and Sedatives | GABRD |
| BROMAZEPAM | Hypnotics and Sedatives | GABRE |
| BROMAZEPAM | Hypnotics and Sedatives | GABRG1 |
| BROMAZEPAM | Hypnotics and Sedatives | GABRG2 |
| BROMAZEPAM | Hypnotics and Sedatives | GABRG3 |
| BROMAZEPAM | Hypnotics and Sedatives | GABRP |
| BROMAZEPAM | Hypnotics and Sedatives | GABRQ |
| BROMAZEPAM | Hypnotics and Sedatives | GABRR1 |
| BROMAZEPAM | Hypnotics and Sedatives | GABRR2 |
| BROMAZEPAM | Hypnotics and Sedatives | GABRR3 |
| BROMODIPHENHYDRAMINE | Anti-Allergic Agents | HRH1 |
| BROMPHENIRAMINE | Anti-Allergic Agents | HRH1 |
| BUCLIZINE | Antiemetics | CHRM1 |
| BUCLIZINE | Antiemetics | HRH1 |
| BUMETANIDE | Antihypertensive Agents; Diuretics | SLC12A1 |
| BUMETANIDE | Antihypertensive Agents; Diuretics | SLC12A2 |
| BUMETANIDE | Antihypertensive Agents; Diuretics | SLC12A4 |
| BUMETANIDE | Antihypertensive Agents; Diuretics | SLC12A5 |
| BUSPIRONE | Anti-anxiety Agents | DRD2 |
| BUSPIRONE | Anti-anxiety Agents | HTR1A |
| BUTABARBITAL | Hypnotics and Sedatives | CHRNA4 |
| BUTABARBITAL | Hypnotics and Sedatives | CHRNA7 |
| BUTABARBITAL | Hypnotics and Sedatives | GABRA1 |
| BUTABARBITAL | Hypnotics and Sedatives | GABRA2 |
| BUTABARBITAL | Hypnotics and Sedatives | GABRA3 |
| BUTABARBITAL | Hypnotics and Sedatives | GABRA4 |
| BUTABARBITAL | Hypnotics and Sedatives | GABRA5 |
| BUTABARBITAL | Hypnotics and Sedatives | GABRA6 |
| BUTABARBITAL | Hypnotics and Sedatives | GRIA2 |
| BUTABARBITAL | Hypnotics and Sedatives | GRIK2 |
| BUTALBITAL | Analgesics | CHRNA4 |
| BUTALBITAL | Analgesics | CHRNA7 |
| BUTALBITAL | Analgesics | GABRA1 |
| BUTALBITAL | Analgesics | GABRA2 |
| BUTALBITAL | Analgesics | GABRA3 |
| BUTALBITAL | Analgesics | GABRA4 |
| BUTALBITAL | Analgesics | GABRA5 |
| BUTALBITAL | Analgesics | GABRA6 |
| BUTALBITAL | Analgesics | GRIA2 |
| BUTALBITAL | Analgesics | GRIK2 |
| BUTETHAL | Hypnotics and Sedatives | CHRNA4 |
| BUTETHAL | Hypnotics and Sedatives | CHRNA7 |
| BUTETHAL | Hypnotics and Sedatives | GABRA1 |
| BUTETHAL | Hypnotics and Sedatives | GABRA2 |
| BUTETHAL | Hypnotics and Sedatives | GABRA3 |
| BUTETHAL | Hypnotics and Sedatives | GABRA4 |
| BUTETHAL | Hypnotics and Sedatives | GABRA5 |
| BUTETHAL | Hypnotics and Sedatives | GABRA6 |
| BUTETHAL | Hypnotics and Sedatives | GRIA2 |
| BUTETHAL | Hypnotics and Sedatives | GRIK2 |
| BUTORPHANOL | Analgesics, Opioid | OPRD1 |
| BUTORPHANOL | Analgesics, Opioid | OPRK1 |
| BUTORPHANOL | Analgesics, Opioid | OPRM1 |
| CABERGOLINE | Antiparkinson Agents | DRD2 |
| CAFFEINE | Central Nervous System Stimulants | ADORA1 |
| CAFFEINE | Central Nervous System Stimulants | ADORA2A |
| CAFFEINE | Central Nervous System Stimulants | PDE4B |
| CALCIPOTRIOL | Dermatologic Agents | VDR |
| CANDOXATRIL | Antihypertensive Agents | ACE |
| CANDOXATRIL | Antihypertensive Agents | MME |
| CAPTOPRIL | Antihypertensive Agents | ACE |
| CARBACHOL | Antiglaucomic Agents | CHRM1 |
| CARBACHOL | Antiglaucomic Agents | CHRM2 |
| CARBACHOL | Antiglaucomic Agents | CHRNA2 |
| CARBETOCIN | Labor Inducing Agents | OXTR |
| CARBIMAZOLE | Antithyroid Agents | TPO |
| CARBINOXAMINE | Anti-Allergic Agents | CHRM1 |
| CARBINOXAMINE | Anti-Allergic Agents | HRH1 |
| CARBOPROST | Abortifacient Agents | PTGER1 |
| TROMETHAMINE | | |
| CARPHENAZINE | Antipsychotic Agents | DRD1 |
| CARPHENAZINE | Antipsychotic Agents | DRD2 |
| CARPHENAZINE | Antipsychotic Agents | DRD5 |
| CARPROFEN | Anti-Inflammatory Agents, Non-Steroidal | PTGS2 |
| CARTEOLOL | Antiglaucomic Agents | ADRB1 |
| CARTEOLOL | Antiglaucomic Agents | ADRB2 |
| CERULETIDE | Diagnostic Agents | CCKAR |
| CEVIMELINE | Parasympathomimetics | CHRM1 |
| CEVIMELINE | Parasympathomimetics | CHRM3 |
| CHLOPHEDIANOL | Antitussive Agents | HRH1 |
| CHLORDIAZEPOXIDE | Hypnotics and Sedatives | GABRA1 |
| CHLORDIAZEPOXIDE | Hypnotics and Sedatives | GABRA2 |
| CHLORDIAZEPOXIDE | Hypnotics and Sedatives | GABRA3 |
| CHLORDIAZEPOXIDE | Hypnotics and Sedatives | GABRA4 |
| CHLORDIAZEPOXIDE | Hypnotics and Sedatives | GABRA5 |
| CHLORDIAZEPOXIDE | Hypnotics and Sedatives | GABRA6 |
| CHLORDIAZEPOXIDE | Hypnotics and Sedatives | GABRB1 |
| CHLORDIAZEPOXIDE | Hypnotics and Sedatives | GABRB2 |
| CHLORDIAZEPOXIDE | Hypnotics and Sedatives | GABRB3 |
| CHLORDIAZEPOXIDE | Hypnotics and Sedatives | GABRD |
| CHLORDIAZEPOXIDE | Hypnotics and Sedatives | GABRE |
| CHLORDIAZEPOXIDE | Hypnotics and Sedatives | GABRG1 |
| CHLORDIAZEPOXIDE | Hypnotics and Sedatives | GABRG2 |
| CHLORDIAZEPOXIDE | Hypnotics and Sedatives | GABRG3 |
| CHLORDIAZEPOXIDE | Hypnotics and Sedatives | GABRP |
| CHLORDIAZEPOXIDE | Hypnotics and Sedatives | GABRQ |
| CHLORDIAZEPOXIDE | Hypnotics and Sedatives | GABRR1 |
| CHLORDIAZEPOXIDE | Hypnotics and Sedatives | GABRR2 |
| CHLORDIAZEPOXIDE | Hypnotics and Sedatives | GABRR3 |
| CHLORMERODRIN | Antihypertensive Agents; Diuretics | SLC12A1 |
| CHLORMEZANONE | Anti-anxiety Agents; Muscle Relaxants | BZRP |
| CHLOROPROCAINE | Anesthetics, Local | SCN10A |
| CHLOROTHIAZIDE | Antihypertensive Agents; Diuretics | CA1 |
| CHLOROTHIAZIDE | Antihypertensive Agents; Diuretics | CA2 |
| CHLOROTHIAZIDE | Antihypertensive Agents; Diuretics | CA4 |
| CHLOROTHIAZIDE | Antihypertensive Agents; Diuretics | SLC12A3 |
| CHLOROTRIANISENE | Hormone Replacement Agents | ESR1 |
| CHLORPHENIRAMINE | Anti-Allergic Agents | HRH1 |
| CHLORPROPAMIDE | Hypoglycemic Agents | KCNJ1 |
| CHLORPROTHIXENE | Antipsychotic Agents | DRD1 |
| CHLORPROTHIXENE | Antipsychotic Agents | DRD2 |
| CHLORPROTHIXENE | Antipsychotic Agents | DRD3 |
| CHLORPROTHIXENE | Antipsychotic Agents | HRH1 |
| CHLORPROTHIXENE | Antipsychotic Agents | HTR2A |
| CHLORPROTHIXENE | Antipsychotic Agents | HTR2B |
| CHLORPROTHIXENE | Antipsychotic Agents | HTR2C |
| CHLORTHALIDONE | Antihypertensive Agents; Diuretics | SLC12A1 |
| CHLORZOXAZONE | Muscle Relaxants | KCNMA1 |
| CICLESONIDE | Anti-Inflammatory Agents; Anti-allergic agents; | NR3C1 |
| | Glucocorticoids | |
| CILASTATIN | Adjuvants, enzyme inhibitors | DPEP1 |
| CILAZAPRIL | Antihypertensive Agents | ACE |
| CILOSTAZOL | Platelet Aggregation Inhibitors | PDE3A |
| CIMETIDINE | GI Anti-Ulcer Agents, antihistamines | HRH2 |
| CINACALCET | Calcimimetics | CASR |
| CINALUKAST | Anti-Asthmatic Agents | CYSLTR1 |
| CINNARIZINE | Anti-Allergic Agents | HRH1 |
| CINOLAZEPAM | Hypnotics and Sedatives | GABRA1 |
| CINOLAZEPAM | Hypnotics and Sedatives | GABRA2 |
| CINOLAZEPAM | Hypnotics and Sedatives | GABRA3 |
| CINOLAZEPAM | Hypnotics and Sedatives | GABRA5 |
| CINOLAZEPAM | Hypnotics and Sedatives | GABRB1 |
| CINOLAZEPAM | Hypnotics and Sedatives | GABRB2 |
| CINOLAZEPAM | Hypnotics and Sedatives | GABRB3 |
| CINOLAZEPAM | Hypnotics and Sedatives | GABRD |
| CINOLAZEPAM | Hypnotics and Sedatives | GABRE |
| CINOLAZEPAM | Hypnotics and Sedatives | GABRG1 |
| CINOLAZEPAM | Hypnotics and Sedatives | GABRG2 |
| CINOLAZEPAM | Hypnotics and Sedatives | GABRG3 |
| CINOLAZEPAM | Hypnotics and Sedatives | GABRP |
| CINOLAZEPAM | Hypnotics and Sedatives | GABRR1 |
| CINOLAZEPAM | Hypnotics and Sedatives | GABRR2 |
| CINOLAZEPAM | Hypnotics and Sedatives | GABRR3 |
| CISAPRIDE | Parasympathomimetics | HTR4 |
| CISATRACURIUM | Neuromuscular Blocking Agents | CHRNA2 |
| BESYLATE | | |
| CITALOPRAM | Antidepressive Agents, Second-Generation | SLC6A4 |
| CLEMASTINE | Anti-Allergic Agents | HRH1 |
| CLENBUTEROL | Bronchodilator Agents | ADRB2 |
| CLIDINIUM | GI Anti-Ulcer Agents, anticholinergic; | CHRM1 |
| | Antispasmodics | |
| CLOCORTOLONE | Anti-Inflammatory Agents; Anti-pruritics; | NR3C1 |
| | Corticosteroids, topical | |
| CLOFIBRATE | Anticholesteremic Agents | PPARA |
| CLOMIPRAMINE | Antidepressive Agents, Tricyclic | SLC6A2 |
| CLOMIPRAMINE | Antidepressive Agents, Tricyclic | SLC6A4 |
| CLORAZEPATE | Hypnotics and Sedatives | BZRP |
| CLORAZEPATE | Hypnotics and Sedatives | GABRA1 |
| CLORAZEPATE | Hypnotics and Sedatives | GABRA2 |
| CLORAZEPATE | Hypnotics and Sedatives | GABRA3 |
| CLORAZEPATE | Hypnotics and Sedatives | GABRA4 |
| CLORAZEPATE | Hypnotics and Sedatives | GABRA5 |
| CLORAZEPATE | Hypnotics and Sedatives | GABRA6 |
| CLORAZEPATE | Hypnotics and Sedatives | GABRB1 |
| CLORAZEPATE | Hypnotics and Sedatives | GABRB2 |
| CLORAZEPATE | Hypnotics and Sedatives | GABRB3 |
| CLORAZEPATE | Hypnotics and Sedatives | GABRD |
| CLORAZEPATE | Hypnotics and Sedatives | GABRE |
| CLORAZEPATE | Hypnotics and Sedatives | GABRG1 |
| CLORAZEPATE | Hypnotics and Sedatives | GABRG2 |
| CLORAZEPATE | Hypnotics and Sedatives | GABRG3 |
| CLORAZEPATE | Hypnotics and Sedatives | GABRP |
| CLORAZEPATE | Hypnotics and Sedatives | GABRQ |
| CLORAZEPATE | Hypnotics and Sedatives | GABRR1 |
| CLORAZEPATE | Hypnotics and Sedatives | GABRR2 |
| CLORAZEPATE | Hypnotics and Sedatives | GABRR3 |
| CLOTIAZEPAM | Hypnotics and Sedatives | GABRA1 |
| CLOTIAZEPAM | Hypnotics and Sedatives | GABRA2 |
| CLOTIAZEPAM | Hypnotics and Sedatives | GABRA3 |
| CLOTIAZEPAM | Hypnotics and Sedatives | GABRA5 |
| CLOTIAZEPAM | Hypnotics and Sedatives | GABRB1 |
| CLOTIAZEPAM | Hypnotics and Sedatives | GABRB2 |
| CLOTIAZEPAM | Hypnotics and Sedatives | GABRB3 |
| CLOTIAZEPAM | Hypnotics and Sedatives | GABRD |
| CLOTIAZEPAM | Hypnotics and Sedatives | GABRE |
| CLOTIAZEPAM | Hypnotics and Sedatives | GABRG1 |
| CLOTIAZEPAM | Hypnotics and Sedatives | GABRG2 |
| CLOTIAZEPAM | Hypnotics and Sedatives | GABRG3 |
| CLOTIAZEPAM | Hypnotics and Sedatives | GABRP |
| CLOTIAZEPAM | Hypnotics and Sedatives | GABRR1 |
| CLOTIAZEPAM | Hypnotics and Sedatives | GABRR2 |
| CLOTIAZEPAM | Hypnotics and Sedatives | GABRR3 |
| CLOZAPINE | Antipsychotic Agents | DRD1 |
| CLOZAPINE | Antipsychotic Agents | DRD2 |
| CLOZAPINE | Antipsychotic Agents | DRD4 |
| CLOZAPINE | Antipsychotic Agents | HRH1 |
| CLOZAPINE | Antipsychotic Agents | HRH4 |
| CLOZAPINE | Antipsychotic Agents | HTR1A |
| CLOZAPINE | Antipsychotic Agents | HTR2A |
| CLOZAPINE | Antipsychotic Agents | HTR2C |
| COCAINE | local anesthetic | DRD3 |
| COCAINE | local anesthetic | OPRK1 |
| COCAINE | local anesthetic | SCN10A |
| COCAINE | local anesthetic | SCN11A |
| COCAINE | local anesthetic | SCN5A |
| COCAINE | local anesthetic | SLC6A2 |
| COCAINE | local anesthetic | SLC6A3 |
| COCAINE | local anesthetic | SLC6A4 |
| CODEINE | Analgesics, Opioid; Antitussive Agents | OPRD1 |
| CODEINE | Analgesics, Opioid; Antitussive Agents | OPRK1 |
| CODEINE | Analgesics, Opioid; Antitussive Agents | OPRM1 |
| CONJUGATED ESTROGENS | Hormone Replacement Agents | ESR1 |
| CROMOGLICATE | Anti-Asthmatic Agents | KCNMA1 |
| CYCLIZINE | Antiemetics | HRH1 |
| CYCLOBENZAPRINE | Antidepressive Agents, Tricyclic | HTR2A |
| CYCLOPENTOLATE | Mydriatics | CHRM1 |
| CYCLOTHIAZIDE | Antihypertensive Agents; Diuretics | FXYD2 |
| CYCRIMINE | Antiparkinson Agents | CHRM1 |
| CYPROHEPTADINE | Anti-Allergic Agents; Appetite Stimulant | HRH1 |
| CYPROHEPTADINE | Anti-Allergic Agents; Appetite Stimulant | HTR2A |
| CYPROTERONE | Hypersexuality-inhibiting agents; Antihirsutism | AR |
| | agents | |
| DACARBAZINE | Antineoplastic Agents | POLA2 |
| DALFAMPRIDINE | MS-treatment | KCNA1 |
| DANAZOL | Antiendometriosis Agent, Antineoplastic Agent | ESR1 |
| DANAZOL | Antiendometriosis Agent, Antineoplastic Agent | GNRHR |
| DANAZOL | Antiendometriosis Agent, Antineoplastic Agent | GNRHR2 |
| DANTROLENE | Muscle Relaxants | RYR1 |
| DAPIPRAZOLE | ophthalmological agent | ADRA1A |
| DAPIPRAZOLE | ophthalmological agent | ADRA1B |
| DAPIPRAZOLE | ophthalmological agent | ADRA1D |
| DEBRISOQUIN | Antihypertensive Agents | ADRA1A |
| DEBRISOQUIN | Antihypertensive Agents | ADRA1B |
| DEBRISOQUIN | Antihypertensive Agents | ADRA1D |
| DEBRISOQUIN | Antihypertensive Agents | ADRA2A |
| DEBRISOQUIN | Antihypertensive Agents | ADRA2B |
| DEBRISOQUIN | Antihypertensive Agents | ADRA2C |
| DECAMETHONIUM | Muscle Relaxants | CHRNA2 |
| DEMECARIUM BROMIDE | Antiglaucomic Agents | ACHE |
| DEMECARIUM BROMIDE | Antiglaucomic Agents | BCHE |
| DESERPIDINE | Antihypertensive Agents | ACE |
| DESFLURANE | inhalation anesthetics | ATP2C1 |
| DESFLURANE | inhalation anesthetics | ATP5D |
| DESFLURANE | inhalation anesthetics | GABRA1 |
| DESFLURANE | inhalation anesthetics | GLRA1 |
| DESFLURANE | inhalation anesthetics | GRIA1 |
| DESFLURANE | inhalation anesthetics | KCNA1 |
| DESFLURANE | inhalation anesthetics | MT-ND1 |
| DESIPRAMINE | Antidepressive Agents, Tricyclic | ADRB1 |
| DESIPRAMINE | Antidepressive Agents, Tricyclic | ADRB2 |
| DESIPRAMINE | Antidepressive Agents, Tricyclic | CHRM1 |
| DESIPRAMINE | Antidepressive Agents, Tricyclic | CHRM2 |
| DESIPRAMINE | Antidepressive Agents, Tricyclic | HRH1 |
| DESIPRAMINE | Antidepressive Agents, Tricyclic | SLC6A2 |
| DESIPRAMINE | Antidepressive Agents, Tricyclic | SLC6A4 |
| DESLANOSIDE | Antiarrhythmia Agents; Cardiotonic Agents | ATP1A1 |
| DESOGESTREL | Contraceptives, Oral | ESR1 |
| DESOGESTREL | Contraceptives, Oral | PGR |
| DESOXIMETASONE | Anti-Inflammatory Agents; Glucocorticoids | NR3C1 |
| DESOXYCORTICOSTERONE | Hormone Replacement Agents, anti-addison | NR3C2 |
| PIVALATE | agent | |
| DEXBROMPHENIRAMINE | Anti-Allergic Agents | HRH1 |
| DEXFENFLURAMINE | Appetite Depressants | SLC6A4 |
| DEXMEDETOMIDINE | Analgesics; Hypnotics and Sedatives | ADRA2A |
| DEXTROMETHORPHAN | Antitussive Agents | GRIN3A |
| DEXTROMETHORPHAN | Antitussive Agents | OPRS1 |
| DEZOCINE | Analgesics, Opioid | OPRK1 |
| DEZOCINE | Analgesics, Opioid | OPRM1 |
| DIAZOXIDE | Antihypertensive Agents; Vasodilator Agents | SLC12A3 |
| DIBUCAINE | Anesthetics, Local | SCN10A |
| DIBUCAINE | Anesthetics, Local | SCN5A |
| DICHLORPHENAMIDE | Antiglaucomic Agents | CA1 |
| DICUMAROL | Anticoagulants | VKORC1 |
| DICYCLOMINE | Antispasmodics | CHRM1 |
| DIENESTROL | Hormone Replacement Agents | ESR1 |
| DIETHYLPROPION | Appetite Depressants | SLC6A2 |
| DIETHYLPROPION | Appetite Depressants | SLC6A3 |
| DIETHYLSTILBESTROL | Hormone Replacement Agents | ESR1 |
| DIFLORASONE | Anti-Inflammatory Agents; Glucocorticoids | NR3C1 |
| DIGITOXIN | Anti-Arrhythmia Agents; Cardiotonic Agents | ATP1A1 |
| DIGOXIN | Anti-Arrhythmia Agents; Cardiotonic Agents | ATP1A1 |
| DIHYDROTACHYSTEROL | Anti-migraine Agents | VDR |
| DIMENHYDRINATE | Antiemetics | HRH1 |
| DINOPROST | Abortifacient Agents | PTGIR |
| TROMETHAMINE | | |
| DINOPROSTONE | Abortifacient Agents | PTGER1 |
| DINOPROSTONE | Abortifacient Agents | PTGER2 |
| DINOPROSTONE | Abortifacient Agents | PTGER3 |
| DINOPROSTONE | Abortifacient Agents | PTGER4 |
| DIPHEMANIL | Bronchodilator Agents | CHRM3 |
| METHYLSULFATE | | |
| DIPHENHYDRAMINE | Anti-Allergic Agents; Hypnotics and sedatives; | HRH1 |
| | Antiemetics; Antipruritics; Antitussives | |
| DIPHENIDOL | Antiemetics | CHRM1 |
| DIPHENIDOL | Antiemetics | CHRM2 |
| DIPHENIDOL | Antiemetics | CHRM3 |
| DIPHENOXYLATE | Antidiarrhcals | OPRM1 |
| DIPHENYLPYRALINE | Anti-Allergic Agents | HRH1 |
| DIPIVEFRIN | Ophthalmologicals | ADRA2A |
| DISOPYRAMIDE | Anti-Arrhythmia Agents | SCN5A |
| DISULFIRAM | Alcohol Deterrents | ALDH2 |
| DIVALPROEX SODIUM | Anticonvulsants; Antimanic Agents | ABAT |
| DOBUTAMINE | Cardiotonic Agents | ADRB1 |
| DOFETILIDE | Anti-Arrhythmia Agents | KCNH2 |
| DOFETILIDE | Anti-Arrhythmia Agents | KCNJ12 |
| DOFETILIDE | Anti-Arrhythmia Agents | KCNK2 |
| DOMPERIDONE | Antiemetics | DRD2 |
| DOXACURIUM | Muscle Relaxants | CHRM2 |
| DOXACURIUM | Muscle Relaxants | CHRNA2 |
| DOXACURIUM CHLORIDE | Muscle Relaxants | CHRM2 |
| DOXACURIUM CHLORIDE | Muscle Relaxants | CHRNA2 |
| DOXAZOSIN | Anticholesteremic Agents; Antihypertensive | ADRA1A |
| | Agents; Vasodilator Agents | |
| DOXAZOSIN | Anticholesteremic Agents; Antihypertensive | ADRA1B |
| | Agents; Vasodilator Agents | |
| DOXAZOSIN | Anticholesteremic Agents; Antihypertensive | ADRA1D |
| | Agents; Vasodilator Agents | |
| DOXYLAMINE | Anti-Allergic Agents; Antiemetics; Antitussive | HRH1 |
| | Agents; Hypnotics and Sedatives | |
| DROMOSTANOLONE | Antineoplastic Agents, Hormonal | AR |
| DRONEDARONE | Anti-Arrhythmia Agents | ADRA1A |
| DRONEDARONE | Anti-Arrhythmia Agents | ADRB1 |
| DRONEDARONE | Anti-Arrhythmia Agents | KCNH2 |
| DROPERIDOL | Adjuvants, Anesthesia | DRD2 |
| DUTASTERIDE | Anti-baldness Agents, Antihyperplasia Agents | SRD5A1 |
| DUTASTERIDE | Anti-baldness Agents, Antihyperplasia Agents | SRD5A2 |
| DYCLONINE | Anesthetics, Local | SCN10A |
| DYDROGESTERONE | Antidysmennorheal Agents | PGR |
| DYPHYLLINE | Bronchodilator Agents; Vasodilator Agents | PDE4A |
| DYPHYLLINE | Bronchodilator Agents; Vasodilator Agents | PDE4B |
| DYPHYLLINE | Bronchodilator Agents; Vasodilator Agents | PDE4C |
| DYPHYLLINE | Bronchodilator Agents; Vasodilator Agents | PDE4D |
| DYPHYLLINE | Bronchodilator Agents; Vasodilator Agents | PDE7A |
| DYPHYLLINE | Bronchodilator Agents; Vasodilator Agents | PDE7B |
| ECHOTHIOPHATE IODIDE | Miotics | BCHE |
| EDROPHONIUM | Anti-Arrhythmia Agents; Antidotes | ACHE |
| EMEDASTINE | Anti-Allergic Agents | HRH1 |
| ENCAINIDE | Anti-Arrhythmia Agents | SCN5A |
| ENFLURANE | Anesthetics, Inhalation | ATP2C1 |
| ENFLURANE | Anesthetics, Inhalation | ATP5D |
| ENFLURANE | Anesthetics, Inhalation | GABRA1 |
| ENFLURANE | Anesthetics, Inhalation | GLRA1 |
| ENFLURANE | Anesthetics, Inhalation | GRIA1 |
| ENFLURANE | Anesthetics, Inhalation | KCNA1 |
| ENFLURANE | Anesthetics, Inhalation | KCNMA1 |
| ENFLURANE | Anesthetics, Inhalation | MT-ND1 |
| ENOXIMONE | Cardiotonic Agents; Vasodilator Agents | PDE3A |
| ENPROFYLLINE | Anti-Asthmatic Agents; Antiarrhythmic Agents; | PDE4A |
| | Bronchodilator Agents | |
| ENPROFYLLINE | Anti-Asthmatic Agents; Antiarrhythmic Agents; | PDE4B |
| | Bronchodilator Agents | |
| EPHEDRINE | Central Nervous System Stimulants | ADRA1A |
| EPIRUBICIN | Antineoplastic Agents | CHD1 |
| EPIRUBICIN | Antineoplastic Agents | TOP2A |
| EPOPROSTENOL | Antihypertensive Agents; Platelet Aggregation | PTGIR |
| | Inhibitors | |
| EPROSARTAN | Antihypertensive Agents | AGTR1 |
| ERGOCALCIFEROL | Antihypocalcemic Agents | VDR |
| ERGOLOID MESYLATE | Nootropic Agents; Vasodilator Agents | ADRA1A |
| ERGOLOID MESYLATE | Nootropic Agents; Vasodilator Agents | ADRA2A |
| ERGOTAMINE | Anti-migraine Agents | HTR1B |
| ERGOTAMINE | Anti-migraine Agents | HTR1D |
| ERYTHRITYL | Antianginal Agents; Vasodilator Agents | NPR1 |
| TETRANITRATE | | |
| ERYTHRITYL | Antianginal Agents; Vasodilator Agents | NPR2 |
| TETRANITRATE | | |
| ESMOLOL | Anti-Arrhythmia Agents | ADRB1 |
| ESTAZOLAM | Anti-anxiety Agents; Anticonvulsants | GABRA1 |
| ESTAZOLAM | Anti-anxiety Agents; Anticonvulsants | GABRA2 |
| ESTAZOLAM | Anti-anxicty Agents; Anticonvulsants | GABRA3 |
| ESTAZOLAM | Anti-anxiety Agents; Anticonvulsants | GABRA5 |
| ESTAZOLAM | Anti-anxiety Agents; Anticonvulsants | GABRB1 |
| ESTAZOLAM | Anti-anxiety Agents; Anticonvulsants | GABRB2 |
| ESTAZOLAM | Anti-anxiety Agents; Anticonvulsants | GABRB3 |
| ESTAZOLAM | Anti-anxiety Agents; Anticonvulsants | GABRD |
| ESTAZOLAM | Anti-anxiety Agents; Anticonvulsants | GABRE |
| ESTAZOLAM | Anti-anxiety Agents; Anticonvulsants | GABRG1 |
| ESTAZOLAM | Anti-anxiety Agents; Anticonvulsants | GABRG2 |
| ESTAZOLAM | Anti-anxiety Agents; Anticonvulsants | GABRG3 |
| ESTAZOLAM | Anti-anxiety Agents; Anticonvulsants | GABRP |
| ESTAZOLAM | Anti-anxiety Agents; Anticonvulsants | GABRR1 |
| ESTAZOLAM | Anti-anxiety Agents; Anticonvulsants | GABRR2 |
| ESTAZOLAM | Anti-anxiety Agents; Anticonvulsants | GABRR3 |
| ESTRIOL | Hormone Replacement Agents | ESR1 |
| ESTRONE | Hormone Replacement Agents | ESR1 |
| ETHACRYNIC ACID | Antihypertensive Agents; Diuretics | SLC12A1 |
| ETHOPROPAZINE | Antidyskinetics | CHRM1 |
| ETHOSUXIMIDE | Anticonvulsants | CACNA1G |
| ETHOTOIN | Anticonvulsants | SCN5A |
| ETHOXZOLAMIDE | Antihypertensive Agents, Diuretics; | CA1 |
| | Antiglaucoma agents | |
| ETHYNODIOL DIACETATE | Contraceptives, Oral, Synthetic | ESR1 |
| ETHYNODIOL DIACETATE | Contraceptives, Oral, Synthetic | PGR |
| ETOMIDATE | Anesthetics, Intravenous | ADRA2B |
| ETOMIDATE | Anesthetics, Intravenous | GABRA1 |
| ETOPOSIDE | Antineoplastic Agents | TOP2A |
| EZETIMIBE | Anticholesteremic Agents | NPC1L1 |
| FELBAMATE | Anticonvulsants; Antiepileptics | GRIN2A |
| FELBAMATE | Anticonvulsants; Antiepileptics | GRIN2B |
| FELBAMATE | Anticonvulsants; Antiepileptics | GRIN3A |
| FENCAMFAMINE | Central Nervous System Stimulants | SLC6A3 |
| FENOPROFEN | NSAID | PTGS1 |
| FENOPROFEN | NSAID | PTGS2 |
| FENOTEROL | Bronchodilator Agents; Tocolytic Agents | ADRB2 |
| FLAVOXATE | Antispasmodics | CHRM1 |
| FLAVOXATE | Antispasmodics | CHRM2 |
| FLECAINIDE | Anti-Arrhythmia Agents | SCN5A |
| FLUDIAZEPAM | Anti-anxiety Agents; Anticonvulsants | GABRA1 |
| FLUDIAZEPAM | Anti-anxiety Agents; Anticonvulsants | GABRA2 |
| FLUDIAZEPAM | Anti-anxiety Agents; Anticonvulsants | GABRA3 |
| FLUDIAZEPAM | Anti-anxiety Agents; Anticonvulsants | GABRA5 |
| FLUDIAZEPAM | Anti-anxiety Agents; Anticonvulsants | GABRB1 |
| FLUDIAZEPAM | Anti-anxiety Agents; Anticonvulsants | GABRB2 |
| FLUDIAZEPAM | Anti-anxiety Agents; Anticonvulsants | GABRB3 |
| FLUDIAZEPAM | Anti-anxiety Agents; Anticonvulsants | GABRD |
| FLUDIAZEPAM | Anti-anxiety Agents; Anticonvulsants | GABRE |
| FLUDIAZEPAM | Anti-anxiety Agents; Anticonvulsants | GABRG1 |
| FLUDIAZEPAM | Anti-anxiety Agents; Anticonvulsants | GABRG2 |
| FLUDIAZEPAM | Anti-anxiety Agents; Anticonvulsants | GABRG3 |
| FLUDIAZEPAM | Anti-anxiety Agents; Anticonvulsants | GABRP |
| FLUDIAZEPAM | Anti-anxiety Agents; Anticonvulsants | GABRR1 |
| FLUDIAZEPAM | Anti-anxiety Agents; Anticonvulsants | GABRR2 |
| FLUDIAZEPAM | Anti-anxiety Agents; Anticonvulsants | GABRR3 |
| FLUDROCORTISONE | Anti-Inflammatory Agents; corticosteroid | NR3C2 |
| FLUMAZENIL | Antidotes, Benzodoazepine Overdose | GABRA1 |
| FLUMAZENIL | Antidotes, Benzodoazepine Overdose | GABRA2 |
| FLUMAZENIL | Antidotes, Benzodoazepine Overdose | GABRA3 |
| FLUMAZENIL | Antidotes, Benzodoazepine Overdose | GABRA5 |
| FLUMETHASONE PIVALATE | Anti-Inflammatory Agents; corticosteroid | NR3C1 |
| FLUNARIZINE | Anticonvulsants; Vasodilator Agents | CACNA1G |
| FLUNARIZINE | Anticonvulsants; Vasodilator Agents | CACNA1H |
| FLUNARIZINE | Anticonvulsants; Vasodilator Agents | CACNA1I |
| FLUNARIZINE | Anticonvulsants; Vasodilator Agents | HRH1 |
| FLUNITRAZEPAM | Hypnotics and Sedatives | BZRP |
| FLUNITRAZEPAM | Hypnotics and Sedatives | GABRA2 |
| FLUNITRAZEPAM | Hypnotics and Sedatives | GABRA3 |
| FLUNITRAZEPAM | Hypnotics and Sedatives | GABRA4 |
| FLUNITRAZEPAM | Hypnotics and Sedatives | GABRA5 |
| FLUNITRAZEPAM | Hypnotics and Sedatives | GABRA6 |
| FLUOROMETHOLONE | Anti-Inflammatory Agents; Anti-allergic agents; | NR3C1 |
| | Glucocorticoids | |
| FLUOXYMESTERONE | Anabolic Agents; Antineoplastic Agents | AR |
| FLUPENTHIXOL | Antipsychotic Agents | DRD1 |
| FLUPENTHIXOL | Antipsychotic Agents | DRD2 |
| FLUPHENAZINE | Antipsychotic Agents | DRD1 |
| FLUPHENAZINE | Antipsychotic Agents | DRD2 |
| FLURANDRENOLIDE | Anti-Inflammatory Agents; Glucocorticoids | NR3C1 |
| FLURAZEPAM | Hypnotics and Sedatives | GABRA1 |
| FLURAZEPAM | Hypnotics and Sedatives | GABRA2 |
| FLURAZEPAM | Hypnotics and Sedatives | GABRA3 |
| FLURAZEPAM | Hypnotics and Sedatives | GABRA4 |
| FLURAZEPAM | Hypnotics and Sedatives | GABRA5 |
| FLURAZEPAM | Hypnotics and Sedatives | GABRA6 |
| FLURAZEPAM | Hypnotics and Sedatives | GABRB1 |
| FLURAZEPAM | Hypnotics and Sedatives | GABRB2 |
| FLURAZEPAM | Hypnotics and Sedatives | GABRB3 |
| FLURAZEPAM | Hypnotics and Sedatives | GABRD |
| FLURAZEPAM | Hypnotics and Sedatives | GABRE |
| FLURAZEPAM | Hypnotics and Sedatives | GABRG1 |
| FLURAZEPAM | Hypnotics and Sedatives | GABRG2 |
| FLURAZEPAM | Hypnotics and Sedatives | GABRG3 |
| FLURAZEPAM | Hypnotics and Sedatives | GABRP |
| FLURAZEPAM | Hypnotics and Sedatives | GABRQ |
| FLURAZEPAM | Hypnotics and Sedatives | GABRR1 |
| FLURAZEPAM | Hypnotics and Sedatives | GABRR2 |
| FLURAZEPAM | Hypnotics and Sedatives | GABRR3 |
| FLUSPIRILENE | Antipsychotic Agents | DRD2 |
| FLUTAMIDE | Antincoplastic Agents, Hormonal | AR |
| FONDAPARINUX | Antithrombotic Agents | SERPINC1 |
| FORASARTAN | Antihypertensive Agents | AGTR1 |
| FOSINOPRIL | Antihypertensive Agents | ACE |
| FUROSEMIDE | Antihypertensive Agents; Diuretics | SLC12A1 |
| GALLAMINE TRIETHIODIDE | Muscle Relaxants, Skeletal | CHRNA2 |
| GEMFIBROZIL | Antilipemic Agents | PPARA |
| GLIBENCLAMIDE | Hypoglycemic Agents | KCNJ1 |
| GLIBENCLAMIDE | Hypoglycemic Agents | KCNJ11 |
| GLICLAZIDE | Hypoglycemic Agents | KCNJ1 |
| GLIPIZIDE | Hypoglycemic Agents | KCNJ1 |
| GLYCODIAZINE | Hypoglycemic Agents | KCNJ1 |
| GONADORELIN | Fertility Agents | GNRHR |
| GONADORELIN | Fertility Agents | GNRHR2 |
| GUANABENZ | Antihypertensive Agents | ADRA2A |
| GUANADREL SULFATE | Antihypertensive Agents | SLC6A2 |
| GUANETHIDINE | Antihypertensive Agents | SLC6A2 |
| HALAZEPAM | Anti-anxiety Agents; Muscle Relaxants; | GABRA1 |
| | Sedative | |
| HALAZEPAM | Anti-anxiety Agents; Muscle Relaxants; | GABRA2 |
| | Sedative | |
| HALAZEPAM | Anti-anxiety Agents; Muscle Relaxants; | GABRA3 |
| | Sedative | |
| HALAZEPAM | Anti-anxiety Agents; Muscle Relaxants; | GABRA5 |
| | Sedative | |
| HALAZEPAM | Anti-anxiety Agents; Muscle Relaxants; | GABRB1 |
| | Sedative | |
| HALAZEPAM | Anti-anxiety Agents; Muscle Relaxants; | GABRB2 |
| | Sedative | |
| HALAZEPAM | Anti-anxiety Agents; Muscle Relaxants; | GABRB3 |
| | Sedative | |
| HALAZEPAM | Anti-anxiety Agents; Muscle Relaxants; | GABRD |
| | Sedative | |
| HALAZEPAM | Anti-anxiety Agents; Muscle Relaxants; | GABRE |
| | Sedative | |
| HALAZEPAM | Anti-anxiety Agents; Muscle Relaxants; | GABRG1 |
| | Sedative | |
| HALAZEPAM | Anti-anxiety Agents; Muscle Relaxants; | GABRG2 |
| | Sedative | |
| HALAZEPAM | Anti-anxiety Agents; Muscle Relaxants; | GABRG3 |
| | Sedative | |
| HALAZEPAM | Anti-anxiety Agents; Muscle Relaxants; | GABRP |
| | Sedative | |
| HALAZEPAM | Anti-anxiety Agents; Muscle Relaxants; | GABRR1 |
| | Sedative | |
| HALAZEPAM | Anti-anxiety Agents; Muscle Relaxants; | GABRR2 |
| | Sedative | |
| HALAZEPAM | Anti-anxiety Agents; Muscle Relaxants; | GABRR3 |
| | Sedative | |
| HALOBETASOL | Anti-inflammatory Agents | NR3C1 |
| PROPIONATE | | |
| HALOPERIDOL | Antipsychotic Agents | DRD2 |
| HALOTHANE | Anesthetics, Inhalation | ATP5D |
| HEXAFLURONIUM | Muscle Relaxants | BCHE |
| BROMIDE | | |
| HEXOBARBITAL | Hypnotics and Sedatives | CHRNA4 |
| HEXOBARBITAL | Hypnotics and Sedatives | CHRNA7 |
| HEXOBARBITAL | Hypnotics and Sedatives | GABRA1 |
| HEXOBARBITAL | Hypnotics and Sedatives | GABRA2 |
| HEXOBARBITAL | Hypnotics and Sedatives | GABRA3 |
| HEXOBARBITAL | Hypnotics and Sedatives | GABRA4 |
| HEXOBARBITAL | Hypnotics and Sedatives | GABRA5 |
| HEXOBARBITAL | Hypnotics and Sedatives | GABRA6 |
| HEXOBARBITAL | Hypnotics and Sedatives | GRIA2 |
| HEXOBARBITAL | Hypnotics and Sedatives | GRIK2 |
| HEXYLCAINE | Anesthetics, Local | SCN10A |
| HEXYLCAINE | Anesthetics, Local | SCN5A |
| HOMATROPINE | GI Anti-Ulcer Agents, Antimuscarinics | CHRM1 |
| METHYLBROMIDE | | |
| HOMATROPINE | GI Anti-Ulcer Agents, Antimuscarinics | CHRM2 |
| METHYLBROMIDE | | |
| HOMATROPINE | GI Anti-Ulcer Agents, Antimuscarinics | CHRM3 |
| METHYLBROMIDE | | |
| HOMATROPINE | GI Anti-Ulcer Agents, Antimuscarinics | CHRM4 |
| METHYLBROMIDE | | |
| HOMATROPINE | GI Anti-Ulcer Agents, Antimuscarinics | CHRM5 |
| METHYLBROMIDE | | |
| HYDROCORTAMATE | Anti-Inflammatory Agents; Glucocorticoids | NR3C1 |
| HYDROCORTAMATE | Anti-Inflammatory Agents; Glucocorticoids | NR3C1 |
| HYDROFLUMETHIAZIDE | Antihypertensive Agents; Diuretics | SLC12A1 |
| HYDROXYUREA | Antineoplastic Agents | RRM1 |
| HYDROXYZINE | Antipruritics; Anxiolytics sedatives and | HRH1 |
| | hypnotics | |
| IBUTILIDE | Anti-Arrhythmia Agents | CACNA1C |
| IBUTILIDE | Anti-Arrhythmia Agents | CACNA2D1 |
| IBUTILIDE | Anti-Arrhythmia Agents | CACNB1 |
| IBUTILIDE | Anti-Arrhythmia Agents | KCNH2 |
| IDARUBICIN | Antineoplastic Agents | TOP2A |
| IFOSFAMIDE | Antineoplastic Agents | DNMT1 |
| IMIPRAMINE | Antidepressive Agents, Tricyclic | SLC6A2 |
| IMIPRAMINE | Antidepressive Agents, Tricyclic | SLC6A4 |
| INDAPAMIDE | Antihypertensive Agents; Diuretics | KCNE1 |
| INDAPAMIDE | Antihypertensive Agents; Diuretics | KCNQ1 |
| INDECAINIDE | Anti-Arrhythmia Agents | SCN5A |
| ISOCARBOXAZID | Antidepressive Agents | MAOA |
| ISOCARBOXAZID | Antidepressive Agents | MAOB |
| ISOETHARINE | Bronchodilator Agents | ADRB1 |
| ISOFLURANE | Anesthetics, Inhalation | ATP2C1 |
| ISOFLURANE | Anesthetics, Inhalation | GABRA1 |
| ISOFLURANE | Anesthetics, Inhalation | GLRA1 |
| ISOFLURANE | Anesthetics, Inhalation | GRIA1 |
| ISOFLURANE | Anesthetics, Inhalation | KCNA1 |
| ISOFLUROPHATE | Antiglaucomic Agents | BCHE |
| ISOPROTERENOL | Bronchodilator Agents; Cardiotonic Agents | ADRB1 |
| ISOPROTERENOL | Bronchodilator Agents; Cardiotonic Agents | ADRB2 |
| ISOSORBIDE-5- | Antianginal Agents; Vasodilator Agents | NPR1 |
| MONONITRATE | | |
| ISRADIPINE | Antihypertensive Agents; Vasodilator Agents | CACNA1C |
| ISRADIPINE | Antihypertensive Agents; Vasodilator Agents | CACNA2D1 |
| LABETALOL | Antihypertensive Agents | ADRA1A |
| LABETALOL | Antihypertensive Agents | ADRA1B |
| LABETALOL | Antihypertensive Agents | ADRB1 |
| LABETALOL | Antihypertensive Agents | ADRB2 |
| LEFLUNOMIDE | Antirheumatic Agents | DHODH |
| LEVALLORPHAN | Opiate Antagonists | OPRM1 |
| LEVOBUNOLOL | Antiglaucomic Agents | ADRB1 |
| LEVOBUNOLOL | Antiglaucomic Agents | ADRB2 |
| LEVOBUPIVACAINE | Anesthetics, Local | SCN10A |
| LEVOCABASTINE | Anti-Allergic Agents | HRH1 |
| LEVOMETHADYL ACETATE | Analgesics, Opioid | OPRM1 |
| LEVORPHANOL | Analgesics, Opioid | OPRM1 |
| LIOTHYRONINE | Hormone Replacement Agents | THRA |
| LIOTHYRONINE | Hormone Replacement Agents | THRB |
| LISDEXAMFETAMINE | Central Nervous System Stimulants | ADRA1A |
| LISDEXAMFETAMINE | Central Nervous System Stimulants | ADRA1B |
| LISDEXAMFETAMINE | Central Nervous System Stimulants | SLC6A3 |
| LISURIDE | Antiparkinson Agents | DRD1 |
| LISURIDE | Antiparkinson Agents | DRD2 |
| LISURIDE | Antiparkinson Agents | HTR1A |
| LOPERAMIDE | Antidiarrheals | OPRM1 |
| LORAZEPAM | Anti-anxiety Agents; Anticonvulsants; | BZRP |
| | Hypnotics and Sedatives | |
| LORAZEPAM | Anti-anxiety Agents; Anticonvulsants; | GABRA1 |
| | Hypnotics and Sedatives | |
| LORAZEPAM | Anti-anxiety Agents; Anticonvulsants; | GABRA2 |
| | Hypnotics and Sedatives | |
| LORAZEPAM | Anti-anxicty Agents; Anticonvulsants; | GABRA3 |
| | Hypnotics and Sedatives | |
| LORAZEPAM | Anti-anxiety Agents; Anticonvulsants; | GABRA4 |
| | Hypnotics and Sedatives | |
| LORAZEPAM | Anti-anxiety Agents; Anticonvulsants; | GABRA5 |
| | Hypnotics and Sedatives | |
| LORAZEPAM | Anti-anxiety Agents; Anticonvulsants; | GABRA6 |
| | Hypnotics and Sedatives | |
| LORAZEPAM | Anti-anxiety Agents; Anticonvulsants; | GABRB1 |
| | Hypnotics and Sedatives | |
| LORAZEPAM | Anti-anxiety Agents; Anticonvulsants; | GABRB2 |
| | Hypnotics and Sedatives | |
| LORAZEPAM | Anti-anxiety Agents; Anticonvulsants; | GABRB3 |
| | Hypnotics and Sedatives | |
| LORAZEPAM | Anti-anxiety Agents; Anticonvulsants; | GABRD |
| | Hypnotics and Sedatives | |
| LORAZEPAM | Anti-anxiety Agents; Anticonvulsants; | GABRE |
| | Hypnotics and Sedatives | |
| LORAZEPAM | Anti-anxiety Agents; Anticonvulsants; | GABRG1 |
| | Hypnotics and Sedatives | |
| LORAZEPAM | Anti-anxiety Agents; Anticonvulsants; | GABRG2 |
| | Hypnotics and Sedatives | |
| LORAZEPAM | Anti-anxiety Agents; Anticonvulsants; | GABRG3 |
| | Hypnotics and Sedatives | |
| LORAZEPAM | Anti-anxiety Agents; Anticonvulsants; | GABRP |
| | Hypnotics and Sedatives | |
| LORAZEPAM | Anti-anxiety Agents; Anticonvulsants; | GABRQ |
| | Hypnotics and Sedatives | |
| LORAZEPAM | Anti-anxiety Agents; Anticonvulsants; | GABRR1 |
| | Hypnotics and Sedatives | |
| LORAZEPAM | Anti-anxiety Agents; Anticonvulsants; | GABRR2 |
| | Hypnotics and Sedatives | |
| LORAZEPAM | Anti-anxiety Agents; Anticonvulsants; | GABRR3 |
| | Hypnotics and Sedatives | |
| LOSARTAN | Antihypertensive Agents | AGTR1 |
| MAPROTILINE | Antidepressive Agents, Second-Generation | SLC6A2 |
| MARIMASTAT | Antineoplastic Agents | MMP2 |
| MARIMASTAT | Antineoplastic Agents | MMP3 |
| MARIMASTAT | Antineoplastic Agents | MMP9 |
| MARINOL | Antiemetics | CNR1 |
| MECLIZINE | Antiemetics | HRH1 |
| MECLOFENAMIC ACID | NSAID | ALOX5 |
| MECLOFENAMIC ACID | NSAID | PTGS1 |
| MECLOFENAMIC ACID | NSAID | PTGS2 |
| MEDRYSONE | Anti-Inflammatory Agents, Topical | NR3C1 |
| MEFENAMIC ACID | NSAID | PTGS1 |
| MEFENAMIC ACID | NSAID | PTGS2 |
| MEGESTROL | Antineoplastic Agents, Hormonal; | ESR1 |
| | Contraceptives | |
| MEGESTROL | Antineoplastic Agents, Hormonal; | PGR |
| | Contraceptives | |
| MELATONIN | Hypnotics and Sedatives | MTNR1A |
| MELATONIN | Hypnotics and Sedatives | MTNR1B |
| MELOXICAM | NSAID | PTGS2 |
| MENTHOL | Antipruritics | TRPA1 |
| MENTHOL | Antipruritics | TRPM8 |
| MENTHOL | Antipruritics | TRPV3 |
| MEPENZOLATE | Antispasmodics | GPR109A |
| MEPENZOLATE | Antispasmodics | GPR109B |
| MEPERIDINE | Analgesics, Opioid | OPRK1 |
| MEPHENTERMINE | Antihypotensive Agents; Vasoconstrictor | ADRA1A |
| | Agents | |
| MEPHENYTOIN | Anticonvulsants | SCN5A |
| MEPROBAMATE | Anticonvulsants; Hypnotics and Sedatives | GABRA1 |
| MEPROBAMATE | Anticonvulsants; Hypnotics and Sedatives | GABRA2 |
| MEPROBAMATE | Anticonvulsants; Hypnotics and Sedatives | GABRA3 |
| MEPROBAMATE | Anticonvulsants; Hypnotics and Sedatives | GABRA4 |
| MEPROBAMATE | Anticonvulsants; Hypnotics and Sedatives | GABRA5 |
| MEPROBAMATE | Anticonvulsants; Hypnotics and Sedatives | GABRA6 |
| MEQUITAZINE | Anti-Allergic Agents | HRH1 |
| MERCAPTOPURINE | Antineoplastic Agents | HPRT1 |
| MESORIDAZINE | Antipsychotic Agents | DRD2 |
| MESORIDAZINE | Antipsychotic Agents | HTR2A |
| MESTRANOL | Contraceptives, Oral | ESR1 |
| METARAMINOL | Antihypotensive Agents; Vasoconstrictor | ADRA1A |
| | Agents | |
| METHADONE | Analgesics, Opioid; Antitussive Agents | OPRM1 |
| METHADYL ACETATE | Analgesics, Opioid | OPRM1 |
| METHANTHELINE | GI Anti-Ulcer Agents, anticholinergic; | CHRM1 |
| | Antispasmodics | |
| METHARBITAL | Anticonvulsants | CHRNA4 |
| METHARBITAL | Anticonvulsants | CHRNA7 |
| METHARBITAL | Anticonvulsants | GABRA1 |
| METHARBITAL | Anticonvulsants | GABRA2 |
| METHARBITAL | Anticonvulsants | GABRA3 |
| METHARBITAL | Anticonvulsants | GABRA4 |
| METHARBITAL | Anticonvulsants | GABRA5 |
| METHARBITAL | Anticonvulsants | GABRA6 |
| METHARBITAL | Anticonvulsants | GRIA2 |
| METHARBITAL | Anticonvulsants | GRIK2 |
| METHAZOLAMIDE | Antihypertensive Agents, Diuretics; | CA1 |
| | Antiglaucoma agents | |
| METHDILAZINE | Anti-Allergic Agents | HRH1 |
| METHIMAZOLE | Antithyroid Agents | TPO |
| METHOHEXITAL | Anesthetics, Intravenous | GABRA1 |
| METHOTRIMEPRAZINE | Antipsychotic Agents | ADRA1A |
| METHOTRIMEPRAZINE | Antipsychotic Agents | ADRA1B |
| METHOTRIMEPRAZINE | Antipsychotic Agents | ADRA1D |
| METHOTRIMEPRAZINE | Antipsychotic Agents | CHRM1 |
| METHOTRIMEPRAZINE | Antipsychotic Agents | CHRM2 |
| METHOTRIMEPRAZINE | Antipsychotic Agents | CHRM3 |
| METHOTRIMEPRAZINE | Antipsychotic Agents | CHRM4 |
| METHOTRIMEPRAZINE | Antipsychotic Agents | CHRM5 |
| METHOTRIMEPRAZINE | Antipsychotic Agents | DRD3 |
| METHOTRIMEPRAZINE | Antipsychotic Agents | HRH1 |
| METHOTRIMEPRAZINE | Antipsychotic Agents | HTR2B |
| METHOXAMINE | Antihypotensive Agents; Vasoconstrictor | ADRA1A |
| | Agents | |
| METHOXAMINE | Antihypotensive Agents; Vasoconstrictor | ADRA1B |
| | Agents | |
| METHOXYFLURANE | Anesthetics, Inhalation | ATP5D |
| METHYCLOTHIAZIDE | Antihypertensive Agents; Diuretics | SLC12A1 |
| METHYLDOPA | Antihypertensive Agents | ADRA2A |
| METHYLERGONOVINE | Abortifacient Agents | DRD1 |
| METHYLNALTREXONE | OIC treatment | OPRM1 |
| BROMIDE | | |
| METHYLPHENOBARBITAL | Anticonvulsants; Hypnotics and Sedatives | CHRNA4 |
| METHYLPHENOBARBITAL | Anticonvulsants; Hypnotics and Sedatives | CHRNA7 |
| METHYLPHENOBARBITAL | Anticonvulsants; Hypnotics and Sedatives | GABRA1 |
| METHYLPHENOBARBITAL | Anticonvulsants; Hypnotics and Sedatives | GABRA2 |
| METHYLPHENOBARBITAL | Anticonvulsants; Hypnotics and Sedatives | GABRA3 |
| METHYLPHENOBARBITAL | Anticonvulsants; Hypnotics and Sedatives | GABRA4 |
| METHYLPHENOBARBITAL | Anticonvulsants; Hypnotics and Sedatives | GABRA5 |
| METHYLPHENOBARBITAL | Anticonvulsants; Hypnotics and Sedatives | GABRA6 |
| METHYLPHENOBARBITAL | Anticonvulsants; Hypnotics and Sedatives | GRIA2 |
| METHYLPHENOBARBITAL | Anticonvulsants; Hypnotics and Sedatives | GRIK2 |
| METHYLPREDNISOLONE | Anti-Inflammatory Agents; Glucocorticoids | NR3C1 |
| METHYLPREDNISOLONE | Anti-Inflammatory Agents; Glucocorticoids | NR3C1 |
| METHYLSCOPOLAMINE | Antispasmodics | CHRM1 |
| METHYPRYLON | Hypnotics and Sedatives | GABRA1 |
| METHYSERGIDE | Anti-migraine agents; Vasoconstrictor Agents | HTR1A |
| METHYSERGIDE | Anti-migraine agents; Vasoconstrictor Agents | HTR2A |
| METHYSERGIDE | Anti-migraine agents; Vasoconstrictor Agents | HTR2C |
| METHYSERGIDE | Anti-migraine agents; Vasoconstrictor Agents | HTR7 |
| METIPRANOLOL | Anti-Arrhythmia Agents; Antihypertensive | ADRB1 |
| | Agents; Anti-glaucoma agent | |
| METIPRANOLOL | Anti-Arrhythmia Agents; Antihypertensive | ADRB2 |
| | Agents; Anti-glaucoma agent | |
| METIXENE | Antiparkinson Agents | CHRM1 |
| METIXENE | Antiparkinson Agents | CHRM2 |
| METIXENE | Antiparkinson Agents | CHRM3 |
| METIXENE | Antiparkinson Agents | CHRM4 |
| METIXENE | Antiparkinson Agents | CHRM5 |
| METOCURINE | Muscle Relaxants | CHRNA2 |
| METOCURINE IODIDE | Muscle Relaxants | CHRNA2 |
| METOLAZONE | Antihypertensive Agents; Diuretics | SLC12A1 |
| METOLAZONE | Antihypertensive Agents; Diuretics | SLC12A3 |
| METOPROLOL | Anti-Arrhythmia Agents; Antihypertensive | ADRB1 |
| | Agents | |
| METYRAPONE | Diagnostic Agents | CYP11B1 |
| METYROSINE | Catecholamine synthesis inhibitors | TH |
| MEXILETINE | Anti-Arrhythmia Agents | SCN5A |
| MIANSERIN | Antidepressive Agents, Second-Generation | ADRA2A |
| MIANSERIN | Antidepressive Agents, Second-Generation | HRH1 |
| MIANSERIN | Antidepressive Agents, Second-Generation | HTR2A |
| MIANSERIN | Antidepressive Agents, Second-Generation | HTR2C |
| MIDAZOLAM | Adjuvants, Anesthesia; Hypnotics and Sedatives | GABRA1 |
| MIDAZOLAM | Adjuvants, Anesthesia; Hypnotics and Sedatives | GABRA2 |
| MIDAZOLAM | Adjuvants, Anesthesia; Hypnotics and Sedatives | GABRA3 |
| MIDAZOLAM | Adjuvants, Anesthesia; Hypnotics and Sedatives | GABRA4 |
| MIDAZOLAM | Adjuvants, Anesthesia; Hypnotics and Sedatives | GABRA5 |
| MIDAZOLAM | Adjuvants, Anesthesia; Hypnotics and Sedatives | GABRA6 |
| MIDAZOLAM | Adjuvants, Anesthesia; Hypnotics and Sedatives | GABRB1 |
| MIDAZOLAM | Adjuvants, Anesthesia; Hypnotics and Sedatives | GABRB2 |
| MIDAZOLAM | Adjuvants, Anesthesia; Hypnotics and Sedatives | GABRB3 |
| MIDAZOLAM | Adjuvants, Anesthesia; Hypnotics and Sedatives | GABRD |
| MIDAZOLAM | Adjuvants, Anesthesia; Hypnotics and Sedatives | GABRE |
| MIDAZOLAM | Adjuvants, Anesthesia; Hypnotics and Sedatives | GABRG1 |
| MIDAZOLAM | Adjuvants, Anesthesia; Hypnotics and Sedatives | GABRG2 |
| MIDAZOLAM | Adjuvants, Anesthesia; Hypnotics and Sedatives | GABRG3 |
| MIDAZOLAM | Adjuvants, Anesthesia; Hypnotics and Sedatives | GABRP |
| MIDAZOLAM | Adjuvants, Anesthesia; Hypnotics and Sedatives | GABRQ |
| MIDAZOLAM | Adjuvants, Anesthesia; Hypnotics and Sedatives | GABRR1 |
| MIDAZOLAM | Adjuvants, Anesthesia; Hypnotics and Sedatives | GABRR2 |
| MIDAZOLAM | Adjuvants, Anesthesia; Hypnotics and Sedatives | GABRR3 |
| MIDODRINE | Antihypotensive Agents; Vasoconstrictor | ADRA1A |
| | Agents | |
| MIDODRINE | Antihypotensive Agents; Vasoconstrictor | ADRA1B |
| | Agents | |
| MIGLITOL | Hypoglycemic Agents | MGAM |
| MILRINONE | Cardiotonic Agents; Vasodilator Agents | PDE3A |
| MILRINONE | Cardiotonic Agents; Vasodilator Agents | PDE4A |
| MINAPRINE | Antidepressive Agents | DRD1 |
| MINAPRINE | Antidepressive Agents | DRD2 |
| MINAPRINE | Antidepressive Agents | HTR2A |
| MINAPRINE | Antidepressive Agents | HTR2B |
| MINAPRINE | Antidepressive Agents | HTR2C |
| MINAPRINE | Antidepressive Agents | SLC6A4 |
| MINOXIDIL | Antihypertensive Agents; Vasodilator Agents | KCNJ1 |
| MIVACURIUM | Muscle Relaxants | CHRM2 |
| MIVACURIUM | Muscle Relaxants | CHRNA2 |
| MOEXIPRIL | Antihypertensive Agents | ACE |
| MOLINDONE | Antipsychotic Agents | DRD2 |
| MORICIZINE | Anti-Arrhythmia Agents | SCN5A |
| NABUMETONE | Anti-Inflammatory Agents, Non-Steroidal | PTGS1 |
| NABUMETONE | Anti-Inflammatory Agents, Non-Steroidal | PTGS2 |
| NADOLOL | Anti-Arrhythmia Agents; Antihypertensive | ADRB1 |
| | Agents | |
| NADOLOL | Anti-Arrhythmia Agents; Antihypertensive | ADRB2 |
| | Agents | |
| NAFARELIN | Antiendometriosis Agent | GNRHR |
| NAFARELIN | Antiendometriosis Agent | GNRHR2 |
| NANDROLONE | Antianemic Agents; anti-osteoporosis agents | AR |
| NEDOCROMIL | Anti-Allergic Agents; Anti-Asthmatic Agents | CYSLTR1 |
| NEFAZODONE | Antidepressive Agents, Second-Generation | ADRA1A |
| NEFAZODONE | Antidepressive Agents, Second-Generation | ADRA1B |
| NEFAZODONE | Antidepressive Agents, Second-Generation | HTR2A |
| NEFAZODONE | Antidepressive Agents, Second-Generation | SLC6A2 |
| NEFAZODONE | Antidepressive Agents, Second-Generation | SLC6A4 |
| NEOSTIGMINE | Parasympathomimetics | ACHE |
| NEPAFENAC | NSAID | PTGS1 |
| NEPAFENAC | NSAID | PTGS2 |
| NICARDIPINE | Anti-Arrhythmia Agents; Antihypertensive | CACNA1C |
| | Agents | |
| NICERGOLINE | Nootropic Agents; Vasodilator Agents | ADRA1A |
| NICOTINE | Central Nervous System Stimulants | CHRNA10 |
| NICOTINE | Central Nervous System Stimulants | CHRNA2 |
| NICOTINE | Central Nervous System Stimulants | CHRNA4 |
| NICOTINE | Central Nervous System Stimulants | CHRNA7 |
| NICOTINE | Central Nervous System Stimulants | CHRNA9 |
| NICOTINE | Central Nervous System Stimulants | CHRNB2 |
| NIFEDIPINE | Antianginal Agents; Vasodilator Agents | CACNA2D1 |
| NIFLUMIC ACID | NSAID | PLA2G1B |
| NIFLUMIC ACID | NSAID | PTGS2 |
| NILUTAMIDE | Antineoplastic Agents | AR |
| NIMODIPINE | Antihypertensive Agents; Vasodilator Agents | CACNG1 |
| NISOLDIPINE | Antihypertensive Agents; Vasodilator Agents | CACNA1A |
| NITRAZEPAM | Anticonvulsants; Hypnotics and Sedatives | GABRA1 |
| NITRAZEPAM | Anticonvulsants; Hypnotics and Sedatives | GABRA2 |
| NITRAZEPAM | Anticonvulsants; Hypnotics and Sedatives | GABRA3 |
| NITRAZEPAM | Anticonvulsants; Hypnotics and Sedatives | GABRA4 |
| NITRAZEPAM | Anticonvulsants; Hypnotics and Sedatives | GABRA5 |
| NITRAZEPAM | Anticonvulsants; Hypnotics and Sedatives | GABRA6 |
| NITRAZEPAM | Anticonvulsants; Hypnotics and Sedatives | GABRB1 |
| NITRAZEPAM | Anticonvulsants; Hypnotics and Sedatives | GABRB2 |
| NITRAZEPAM | Anticonvulsants; Hypnotics and Sedatives | GABRB3 |
| NITRAZEPAM | Anticonvulsants; Hypnotics and Sedatives | GABRD |
| NITRAZEPAM | Anticonvulsants; Hypnotics and Sedatives | GABRE |
| NITRAZEPAM | Anticonvulsants; Hypnotics and Sedatives | GABRG1 |
| NITRAZEPAM | Anticonvulsants; Hypnotics and Sedatives | GABRG2 |
| NITRAZEPAM | Anticonvulsants; Hypnotics and Sedatives | GABRG3 |
| NITRAZEPAM | Anticonvulsants; Hypnotics and Sedatives | GABRP |
| NITRAZEPAM | Anticonvulsants; Hypnotics and Sedatives | GABRQ |
| NITRAZEPAM | Anticonvulsants; Hypnotics and Sedatives | GABRR1 |
| NITRAZEPAM | Anticonvulsants; Hypnotics and Sedatives | GABRR2 |
| NITRAZEPAM | Anticonvulsants; Hypnotics and Sedatives | GABRR3 |
| NITRAZEPAM | Anticonvulsants; Hypnotics and Sedatives | SCN1A |
| NITRENDIPINE | Antihypertensive Agents; Vasodilator Agents | CACNG1 |
| NITROPRUSSIDE | Antihypertensive Agents; Vasodilator Agents | NPR1 |
| NIZATIDINE | GI Anti-Ulcer Agents, antihistamines | HRH2 |
| NOREPINEPHRINE | Antihypotensive Agents; Vasoconstrictor | ADRA1A |
| | Agents | |
| NOREPINEPHRINE | Antihypotensive Agents; Vasoconstrictor | ADRA1B |
| | Agents | |
| NOREPINEPHRINE | Antihypotensive Agents; Vasoconstrictor | ADRA1D |
| | Agents | |
| NOREPINEPHRINE | Antihypotensive Agents; Vasoconstrictor | ADRA2A |
| | Agents | |
| NOREPINEPHRINE | Antihypotensive Agents; Vasoconstrictor | ADRA2B |
| | Agents | |
| NOREPINEPHRINE | Antihypotensive Agents; Vasoconstrictor | ADRA2C |
| | Agents | |
| NORETHINDRONE | Contraceptives, Oral, Synthetic | PGR |
| NORGESTIMATE | Contraceptives, Oral, Synthetic | ESR1 |
| NORGESTIMATE | Contraceptives, Oral, Synthetic | PGR |
| NORGESTREL | Contraceptives, Oral, Synthetic | ESR1 |
| NORGESTREL | Contraceptives, Oral, Synthetic | PGR |
| ORCIPRENALINE | Bronchodilator Agents | ADRB2 |
| ORPHENADRINE | Antiparkinson Agents; Muscle Relaxants, | GRIN1 |
| | Central | |
| ORPHENADRINE | Antiparkinson Agents; Muscle Relaxants, | GRIN2D |
| | Central | |
| ORPHENADRINE | Antiparkinson Agents; Muscle Relaxants, | GRIN3A |
| | Central | |
| ORPHENADRINE | Antiparkinson Agents; Muscle Relaxants, | GRIN3B |
| | Central | |
| ORPHENADRINE | Antiparkinson Agents; Muscle Relaxants, | HRH1 |
| | Central | |
| OUABAIN | Cardiotonic Agents | ATP1A1 |
| OXAPROZIN | NSAID | PTGS2 |
| OXAZEPAM | Hypnotics and Sedatives | GABRA1 |
| OXAZEPAM | Hypnotics and Sedatives | GABRA2 |
| OXAZEPAM | Hypnotics and Sedatives | GABRA3 |
| OXAZEPAM | Hypnotics and Sedatives | GABRA4 |
| OXAZEPAM | Hypnotics and Sedatives | GABRA5 |
| OXAZEPAM | Hypnotics and Sedatives | GABRA6 |
| OXAZEPAM | Hypnotics and Sedatives | GABRB1 |
| OXAZEPAM | Hypnotics and Sedatives | GABRB2 |
| OXAZEPAM | Hypnotics and Sedatives | GABRB3 |
| OXAZEPAM | Hypnotics and Sedatives | GABRD |
| OXAZEPAM | Hypnotics and Sedatives | GABRE |
| OXAZEPAM | Hypnotics and Sedatives | GABRG1 |
| OXAZEPAM | Hypnotics and Sedatives | GABRG2 |
| OXAZEPAM | Hypnotics and Sedatives | GABRG3 |
| OXAZEPAM | Hypnotics and Sedatives | GABRP |
| OXAZEPAM | Hypnotics and Sedatives | GABRQ |
| OXAZEPAM | Hypnotics and Sedatives | GABRR1 |
| OXAZEPAM | Hypnotics and Sedatives | GABRR2 |
| OXAZEPAM | Hypnotics and Sedatives | GABRR3 |
| OXPRENOLOL | Antihypertensive Agents; Anti-Arrhythmia | ADRB1 |
| | Agents | |
| OXPRENOLOL | Antihypertensive Agents; Anti-Arrhythmia | ADRB2 |
| | Agents | |
| OXYBUPROCAINE | Anesthetics, Local | SCN10A |
| OXYPHENCYCLIMINE | GI Anti-Ulcer Agents, anticholinergic; | CHRM1 |
| | Antispasmodics | |
| OXYPHENCYCLIMINE | GI Anti-Ulcer Agents, anticholinergic; | CHRM2 |
| | Antispasmodics | |
| OXYPHENCYCLIMINE | GI Anti-Ulcer Agents, anticholinergic; | CHRM3 |
| | Antispasmodics | |
| OXYPHENONIUM | Mydriatics | CHRM1 |
| PAMIDRONATE | Bisphosphonates | FDPS |
| PANCURONIUM | Muscle Relaxants | CHRNA2 |
| PAPAVERINE | Antispasmodics; Anti-impotence Agents; | PDE4B |
| | Vasodilator Agents | |
| PARAMETHADIONE | Anticonvulsants | CACNA1I |
| PARAMETHASONE | Anti-Inflammatory Agents; Glucocorticoids | NR3C1 |
| PEMETREXED | Antineoplastic Agents | DHFR |
| PEMETREXED | Antineoplastic Agents | GART |
| PEMETREXED | Antineoplastic Agents | TYMS |
| PEMIROLAST | Anti-Allergic Agents | HRH1 |
| PENBUTOLOL | Antihypertensive Agents | ADRB1 |
| PENBUTOLOL | Antihypertensive Agents | ADRB2 |
| PENTAGASTRIN | Diagnostic Agents | CCKBR |
| PENTAZOCINE | Analgesics, Opioid | OPRK1 |
| PENTAZOCINE | Analgesics, Opioid | OPRM1 |
| PENTOBARBITAL | Hypnotics and Sedatives | CHRNA4 |
| PENTOBARBITAL | Hypnotics and Sedatives | CHRNA7 |
| PENTOBARBITAL | Hypnotics and Sedatives | GABRA1 |
| PENTOBARBITAL | Hypnotics and Sedatives | GABRA2 |
| PENTOBARBITAL | Hypnotics and Sedatives | GABRA3 |
| PENTOBARBITAL | Hypnotics and Sedatives | GABRA4 |
| PENTOBARBITAL | Hypnotics and Sedatives | GABRA5 |
| PENTOBARBITAL | Hypnotics and Sedatives | GABRA6 |
| PENTOBARBITAL | Hypnotics and Sedatives | GRIA2 |
| PENTOBARBITAL | Hypnotics and Sedatives | GRIK2 |
| PENTOLINIUM | Antihypertensive Agents | CHRNA10 |
| PERGOLIDE | Antiparkinson Agents | DRD1 |
| PERGOLIDE | Antiparkinson Agents | DRD2 |
| PERHEXILINE | Antianginal Agents; Vasodilator Agents | CPT1A |
| PERHEXILINE | Antianginal Agents; Vasodilator Agents | CPT2 |
| PERINDOPRIL | Antihypertensive Agents | ACE |
| PERPHENAZINE | Antipsychotic Agents | DRD1 |
| PERPHENAZINE | Antipsychotic Agents | DRD2 |
| PHENACEMIDE | Anticonvulsants | SCN1A |
| PHENDIMETRAZINE | Appetite Depressants | ADRA1A |
| PHENDIMETRAZINE | Appetite Depressants | ADRA1B |
| PHENELZINE | Antidepressive Agents | MAOA |
| PHENELZINE | Antidepressive Agents | MAOB |
| PHENFORMIN | Hypoglycemic Agents | PRKAA1 |
| PHENINDIONE | Anticoagulants | VKORC1 |
| PHENIRAMINE | Anti-Allergic Agents | HRH1 |
| PHENMETRAZINE | Appetite Depressants | SLC6A2 |
| PHENMETRAZINE | Appctite Depressants | SLC6A3 |
| PHENOBARBITAL | Anticonvulsants; Hypnotics and Sedatives | CHRNA4 |
| PHENOBARBITAL | Anticonvulsants; Hypnotics and Sedatives | CHRNA7 |
| PHENOBARBITAL | Anticonvulsants; Hypnotics and Sedatives | GABRA1 |
| PHENOBARBITAL | Anticonvulsants; Hypnotics and Sedatives | GABRA2 |
| PHENOBARBITAL | Anticonvulsants; Hypnotics and Sedatives | GABRA3 |
| PHENOBARBITAL | Anticonvulsants; Hypnotics and Sedatives | GABRA4 |
| PHENOBARBITAL | Anticonvulsants; Hypnotics and Sedatives | GABRA5 |
| PHENOBARBITAL | Anticonvulsants; Hypnotics and Sedatives | GABRA6 |
| PHENOBARBITAL | Anticonvulsants; Hypnotics and Sedatives | GRIA1 |
| PHENOBARBITAL | Anticonvulsants; Hypnotics and Sedatives | GRIA2 |
| PHENOBARBITAL | Anticonvulsants; Hypnotics and Sedatives | GRIK2 |
| PHENOXYBENZAMINE | Anticonvulsants; Hypnotics and Sedatives | ADRA1A |
| PHENPROCOUMON | Anticoagulants | VKORC1 |
| PHENTERMINE | Appetite Depressants | SLC6A2 |
| PHENTERMINE | Appetite Depressants | SLC6A3 |
| PHENTERMINE | Appetite Depressants | SLC6A4 |
| PHENTOLAMINE | Antihypertensive Agents | ADRA2A |
| PHENYLBUTAZONE | NSAID | PTGIS |
| PHENYLBUTAZONE | NSAID | PTGS2 |
| PHENYLPROPANOLAMINE | Appetite Depressants; Nasal Decongestants | ADRA1A |
| PHENYLPROPANOLAMINE | Appetite Depressants; Nasal Decongestants | ADRA2A |
| PHENYTOIN | Anticonvulsants | SCN1A |
| PHENYTOIN | Anticonvulsants | SCN5A |
| PHYTONADIONE | Antifibrinolytic Agents | GGCX |
| PICROTOXIN | Central Nervous System Stimulants; | GABRA1 |
| | Convulsants | |
| PICROTOXIN | Central Nervous System Stimulants; | GABRR1 |
| | Convulsants | |
| PIMOZIDE | Antidyskinetics; Antipsychotic Agents | DRD2 |
| PINDOLOL | Antihypertensive Agents | ADRB1 |
| PINDOLOL | Antihypertensive Agents | ADRB2 |
| PIPECURONIUM | Muscle Relaxants | CHRNA2 |
| PIRENZEPINE | GI Anti-Ulcer Agents, anticholinergic; | CHRM1 |
| | Antispasmodics | |
| PODOFILOX | Antineoplastic Agents, Phytogenic; Keratolytic | TOP2A |
| | Agents | |
| POLYTHIAZIDE | Antihypertensive Agents; Diuretics | SLC12A3 |
| PRACTOLOL | Anti-Arrhythmia Agents | ADRB1 |
| PRALATREXATE | Antineoplastic Agents | DHFR |
| PRANLUKAST | Anti-Asthmatic Agents | CYSLTR1 |
| PRAZEPAM | Anti-anxiety Agents; Hypnotics and Sedatives | GABRA1 |
| PRAZEPAM | Anti-anxiety Agents; Hypnotics and Sedatives | GABRA2 |
| PRAZEPAM | Anti-anxiety Agents; Hypnotics and Sedatives | GABRA3 |
| PRAZEPAM | Anti-anxiety Agents; Hypnotics and Sedatives | GABRA5 |
| PRAZEPAM | Anti-anxiety Agents; Hypnotics and Sedatives | GABRB1 |
| PRAZEPAM | Anti-anxiety Agents; Hypnotics and Sedatives | GABRB2 |
| PRAZEPAM | Anti-anxiety Agents; Hypnotics and Sedatives | GABRB3 |
| PRAZEPAM | Anti-anxiety Agents; Hypnotics and Sedatives | GABRD |
| PRAZEPAM | Anti-anxiety Agents; Hypnotics and Sedatives | GABRE |
| PRAZEPAM | Anti-anxiety Agents; Hypnotics and Sedatives | GABRG1 |
| PRAZEPAM | Anti-anxiety Agents; Hypnotics and Sedatives | GABRG2 |
| PRAZEPAM | Anti-anxiety Agents; Hypnotics and Sedatives | GABRG3 |
| PRAZEPAM | Anti-anxiety Agents; Hypnotics and Sedatives | GABRP |
| PRAZEPAM | Anti-anxiety Agents; Hypnotics and Sedatives | GABRR1 |
| PRAZEPAM | Anti-anxiety Agents; Hypnotics and Sedatives | GABRR2 |
| PRAZEPAM | Anti-anxiety Agents; Hypnotics and Sedatives | GABRR3 |
| PRAZOSIN | Antihypertensive Agents; antispasmodics | ADRA1A |
| PRAZOSIN | Antihypertensive Agents; antispasmodics | ADRA1B |
| PRAZOSIN | Antihypertensive Agents; antispasmodics | ADRAID |
| PREDNICARBATE | Anti-Inflammatory Agents; Corticosteroids | NR3C1 |
| PRIMIDONE | Anticonvulsants | CHRNA4 |
| PRIMIDONE | Anticonvulsants | CHRNA7 |
| PRIMIDONE | Anticonvulsants | GABRA1 |
| PRIMIDONE | Anticonvulsants | GABRA2 |
| PRIMIDONE | Anticonvulsants | GABRA3 |
| PRIMIDONE | Anticonvulsants | GABRA4 |
| PRIMIDONE | Anticonvulsants | GABRA5 |
| PRIMIDONE | Anticonvulsants | GABRA6 |
| PRIMIDONE | Anticonvulsants | GRIA2 |
| PRIMIDONE | Anticonvulsants | GRIK2 |
| PROBENECID | Uricosuric Agents | SLC22A11 |
| PROBENECID | Uricosuric Agents | SLC22A8 |
| PROCAINAMIDE | Anti-Arrhythmia Agents | SCN5A |
| PROCAINE | Anesthetics, Local | SCN10A |
| PROCATEROL | Bronchodilator Agents | ADRB2 |
| PROCYCLIDINE | Antidyskinetics; Antiparkinson Agents | CHRM1 |
| PROCYCLIDINE | Antidyskinetics; Antiparkinson Agents | CHRM2 |
| PROCYCLIDINE | Antidyskinetics; Antiparkinson Agents | CHRM4 |
| PROGABIDE | Anticonvulsants | GABBR1 |
| PROGABIDE | Anticonvulsants | GABRA1 |
| PROMAZINE | Antiemetics; Antipsychotic Agents | ADRA1A |
| PROMAZINE | Antiemetics; Antipsychotic Agents | ADRA1B |
| PROMAZINE | Antiemetics; Antipsychotic Agents | ADRA1D |
| PROMAZINE | Antiemetics; Antipsychotic Agents | CHRM1 |
| PROMAZINE | Antiemetics; Antipsychotic Agents | CHRM2 |
| PROMAZINE | Antiemetics; Antipsychotic Agents | CHRM3 |
| PROMAZINE | Antiemetics; Antipsychotic Agents | CHRM4 |
| PROMAZINE | Antiemetics; Antipsychotic Agents | CHRM5 |
| PROMAZINE | Antiemetics; Antipsychotic Agents | DRD1 |
| PROMAZINE | Antiemetics; Antipsychotic Agents | DRD2 |
| PROMAZINE | Antiemetics; Antipsychotic Agents | DRD4 |
| PROMAZINE | Antiemetics; Antipsychotic Agents | HRH1 |
| PROMAZINE | Antiemetics; Antipsychotic Agents | HTR2A |
| PROMAZINE | Antiemetics; Antipsychotic Agents | HTR2C |
| PROMETHAZINE | Hypnotics and Sedatives; Anti-anxiety agents; | CHRM1 |
| | Anti-allergic Agents | |
| PROMETHAZINE | Hypnotics and Sedatives; Anti-anxiety agents; | CHRM2 |
| | Anti-allergic Agents | |
| PROMETHAZINE | Hypnotics and Sedatives; Anti-anxiety agents; | CHRM3 |
| | Anti-allergic Agents | |
| PROMETHAZINE | Hypnotics and Sedatives; Anti-anxiety agents; | CHRM4 |
| | Anti-allergic Agents | |
| PROMETHAZINE | Hypnotics and Sedatives; Anti-anxiety agents; | CHRM5 |
| | Anti-allergic Agents | |
| PROMETHAZINE | Hypnotics and Sedatives; Anti-anxiety agents; | HRH1 |
| | Anti-allergic Agents | |
| PROPANTHELINE | GI Anti-Ulcer Agents, anticholinergic; | CHRM1 |
| | Antispasmodics | |
| PROPARACAINE | Anesthetics, Local | SCN10A |
| PROPERICIAZINE | Antipsychotic Agents | ADRA1A |
| PROPERICIAZINE | Antipsychotic Agents | ADRA1B |
| PROPERICIAZINE | Antipsychotic Agents | ADRA1D |
| PROPIOMAZINE | Hypnotics and Sedatives | ADRA1A |
| PROPIOMAZINE | Hypnotics and Sedatives | ADRA1B |
| PROPIOMAZINE | Hypnotics and Sedatives | ADRA1D |
| PROPIOMAZINE | Hypnotics and Sedatives | CHRM1 |
| PROPIOMAZINE | Hypnotics and Sedatives | CHRM2 |
| PROPIOMAZINE | Hypnotics and Sedatives | CHRM3 |
| PROPIOMAZINE | Hypnotics and Sedatives | CHRM4 |
| PROPIOMAZINE | Hypnotics and Sedatives | CHRM5 |
| PROPIOMAZINE | Hypnotics and Sedatives | DRD1 |
| PROPIOMAZINE | Hypnotics and Sedatives | DRD2 |
| PROPIOMAZINE | Hypnotics and Sedatives | DRD4 |
| PROPIOMAZINE | Hypnotics and Sedatives | HRH1 |
| PROPIOMAZINE | Hypnotics and Sedatives | HTR2A |
| PROPIOMAZINE | Hypnotics and Sedatives | HTR2C |
| PROPOXYPHENE | Analgesics, Opioid; Antitussive Agents | OPRD1 |
| PROPOXYPHENE | Analgesics, Opioid; Antitussive Agents | OPRK1 |
| PROPOXYPHENE | Analgesics, Opioid; Antitussive Agents | OPRM1 |
| PROPYLTHIOURACIL | Antithyroid Agents | TPO |
| PROTRIPTYLINE | Antidepressive Agents, Tricyclic | SLC6A2 |
| PROTRIPTYLINE | Antidepressive Agents, Tricyclic | SLC6A4 |
| PYRIDOSTIGMINE | Antimyasthenics | ACHE |
| QUAZEPAM | Hypnotics and Sedatives | GABRA1 |
| QUAZEPAM | Hypnotics and Sedatives | GABRA2 |
| QUAZEPAM | Hypnotics and Sedatives | GABRA3 |
| QUAZEPAM | Hypnotics and Sedatives | GABRA5 |
| QUAZEPAM | Hypnotics and Sedatives | GABRB1 |
| QUAZEPAM | Hypnotics and Sedatives | GABRB3 |
| QUAZEPAM | Hypnotics and Sedatives | GABRD |
| QUAZEPAM | Hypnotics and Sedatives | GABRE |
| QUAZEPAM | Hypnotics and Sedatives | GABRG1 |
| QUAZEPAM | Hypnotics and Sedatives | GABRG2 |
| QUAZEPAM | Hypnotics and Sedatives | GABRG3 |
| QUAZEPAM | Hypnotics and Sedatives | GABRP |
| QUAZEPAM | Hypnotics and Sedatives | GABRR1 |
| QUAZEPAM | Hypnotics and Sedatives | GABRR2 |
| QUAZEPAM | Hypnotics and Sedatives | GABRR3 |
| QUINESTROL | Hormone Replacement Agents | ESR1 |
| QUINETHAZONE | Antihypertensive Agents; Diuretics | SLC12A3 |
| QUINIDINE | Anti-Arrhythmia Agents | SCN5A |
| RALOXIFENE | Hormone Replacement Agents | ESR1 |
| RALOXIFENE | Hormone Replacement Agents | ESR2 |
| RAMIPRIL | Antihypertensive Agents | ACE |
| REMIKIREN | Antihypertensive Agents | REN |
| REMOXIPRIDE | Antipsychotic Agents | DRD2 |
| RESCINNAMINE | Antihypertensive Agents | ACE |
| RESERPINE | Antihypertensive Agents; Antipsychotic Agents | SLC18A2 |
| RIMEXOLONE | Anti-Inflammatory Agents; Corticosteroids | NR3C1 |
| RIMEXOLONE | Anti-Inflammatory Agents; Corticosteroids | NR3C1 |
| RISEDRONATE | Bisphosphonates | FDPS |
| RISPERIDONE | Antipsychotic Agents | DRD2 |
| RISPERIDONE | Antipsychotic Agents | HTR2A |
| RITODRINE | Tocolytic Agents | ADRB2 |
| RIZATRIPTAN | Anti-migraine Agents | HTR1B |
| RIZATRIPTAN | Anti-migraine Agents | HTR1D |
| SALICYLIC ACID | Keratolytic Agents | PTGS1 |
| SALICYLIC ACID | Keratolytic Agents | PTGS2 |
| SALSALATE | Anti-Inflammatory Agents, Non-Steroidal | PTGS1 |
| SALSALATE | Anti-Inflammatory Agents, Non-Steroidal | PTGS2 |
| SAPRISARTAN | Antihypertensive Agents | AGTR1 |
| SAPROPTERIN | PKU-treatment | PAH |
| SCOPOLAMINE | Adjuvants, Anesthesia; Antispasmodics; | CHRM1 |
| | Mydriatics | |
| SECOBARBITAL | Adjuvants, anesthesia; Hypnotics and Sedatives | CHRNA4 |
| SECOBARBITAL | Adjuvants, anesthesia; Hypnotics and Sedatives | CHRNA7 |
| SECOBARBITAL | Adjuvants, anesthesia; Hypnotics and Sedatives | GABRA1 |
| SECOBARBITAL | Adjuvants, anesthesia; Hypnotics and Sedatives | GABRA2 |
| SECOBARBITAL | Adjuvants, anesthesia; Hypnotics and Sedatives | GABRA3 |
| SECOBARBITAL | Adjuvants, anesthesia; Hypnotics and Sedatives | GABRA4 |
| SECOBARBITAL | Adjuvants, anesthesia; Hypnotics and Sedatives | GABRA5 |
| SECOBARBITAL | Adjuvants, anesthesia; Hypnotics and Sedatives | GABRA6 |
| SECOBARBITAL | Adjuvants, anesthesia; Hypnotics and Sedatives | GRIA2 |
| SECOBARBITAL | Adjuvants, anesthesia; Hypnotics and Sedatives | GRIK2 |
| SEVOFLURANE | Anesthetics, Inhalation | ATP2C1 |
| SEVOFLURANE | Anesthetics, Inhalation | ATP5D |
| SEVOFLURANE | Anesthetics, Inhalation | GABRA1 |
| SEVOFLURANE | Anesthetics, Inhalation | GLRA1 |
| SEVOFLURANE | Anesthetics, Inhalation | GRIA1 |
| SEVOFLURANE | Anesthetics, Inhalation | KCNA1 |
| SEVOFLURANE | Anesthetics, Inhalation | MT-ND1 |
| SODIUM TETRADECYL | Sclerosing Agents | PROC |
| SULFATE | | |
| SOTALOL | Anti-Arrhythmia Agents | KCNH2 |
| SPIRAPRIL | Antihypertensive Agents | ACE |
| SUCCINYLCHOLINE | Muscle Relaxants, Skeletal | CHRM1 |
| SULFINPYRAZONE | Uricosuric Agents | ABCC1 |
| SULFINPYRAZONE | Uricosuric Agents | ABCC2 |
| SULINDAC | NSAID | PTGS1 |
| SULINDAC | NSAID | PTGS2 |
| SULPIRIDE | Antidepressive Agents, Second-Generation; | DRD2 |
| | Antipsychotic Agents | |
| SUPROFEN | NSAID | PTGS1 |
| SUPROFEN | NSAID | PTGS2 |
| TACRINE | Nootropic Agents | ACHE |
| TALBUTAL | Analgesics | CHRNA4 |
| TALBUTAL | Analgesics | CHRNA7 |
| TALBUTAL | Analgesics | GABRA1 |
| TALBUTAL | Analgesics | GABRA2 |
| TALBUTAL | Analgesics | GABRA3 |
| TALBUTAL | Analgesics | GABRA4 |
| TALBUTAL | Analgesics | GABRA5 |
| TALBUTAL | Analgesics | GABRA6 |
| TALBUTAL | Analgesics | GRIA2 |
| TALBUTAL | Analgesics | GRIK2 |
| TAMOXIFEN | Antineoplastic Agents, Hormonal | ESR1 |
| TAMOXIFEN | Antineoplastic Agents, Hormonal | ESR2 |
| TASOSARTAN | Antihypertensive Agents | AGTR1 |
| TEMAZEPAM | Hypnotics and Sedatives | BZRP |
| TEMAZEPAM | Hypnotics and Sedatives | GABRA1 |
| TEMAZEPAM | Hypnotics and Sedatives | GABRA2 |
| TEMAZEPAM | Hypnotics and Sedatives | GABRA3 |
| TEMAZEPAM | Hypnotics and Sedatives | GABRA4 |
| TEMAZEPAM | Hypnotics and Sedatives | GABRA5 |
| TEMAZEPAM | Hypnotics and Sedatives | GABRA6 |
| TEMAZEPAM | Hypnotics and Sedatives | GABRB1 |
| TEMAZEPAM | Hypnotics and Sedatives | GABRB2 |
| TEMAZEPAM | Hypnotics and Sedatives | GABRB3 |
| TEMAZEPAM | Hypnotics and Sedatives | GABRD |
| TEMAZEPAM | Hypnotics and Sedatives | GABRE |
| TEMAZEPAM | Hypnotics and Sedatives | GABRG1 |
| TEMAZEPAM | Hypnotics and Sedatives | GABRG2 |
| TEMAZEPAM | Hypnotics and Sedatives | GABRG3 |
| TEMAZEPAM | Hypnotics and Sedatives | GABRP |
| TEMAZEPAM | Hypnotics and Sedatives | GABRQ |
| TEMAZEPAM | Hypnotics and Sedatives | GABRR1 |
| TEMAZEPAM | Hypnotics and Sedatives | GABRR2 |
| TEMAZEPAM | Hypnotics and Sedatives | GABRR3 |
| TENIPOSIDE | Antineoplastic Agents | TOP2A |
| TENOXICAM | NSAID | PTGS1 |
| TENOXICAM | NSAID | PTGS2 |
| TERAZOSIN | Antineoplastic Agents; antihypertensive agents | ADRA1A |
| TERAZOSIN | Antineoplastic Agents; antihypertensive agents | ADRA1B |
| TERAZOSIN | Antineoplastic Agents; antihypertensive agents | ADRA1D |
| TERBUTALINE | Bronchodilator Agents; Tocolytic Agents | ADRB2 |
| TERFENADINE | Anti-Allergic Agents | HRH1 |
| TESTOLACTONE | Antineoplastic Agents, Hormonal | CYP19A1 |
| THIAMYLAL | Anesthetics, Intravenous | GABRA1 |
| THIAMYLAL | Anesthetics, Intravenous | KCNJ11 |
| THIAMYLAL | Anesthetics, Intravenous | KCNJ8 |
| THIETHYLPERAZINE | Antiemetics | CHRM1 |
| THIETHYLPERAZINE | Antiemetics | CHRM2 |
| THIETHYLPERAZINE | Antiemetics | CHRM3 |
| THIETHYLPERAZINE | Antiemetics | CHRM4 |
| THIETHYLPERAZINE | Antiemetics | CHRM5 |
| THIETHYLPERAZINE | Anticmctics | DRD1 |
| THIETHYLPERAZINE | Antiemetics | DRD2 |
| THIETHYLPERAZINE | Antiemetics | DRD4 |
| THIETHYLPERAZINE | Antiemetics | HRH1 |
| THIETHYLPERAZINE | Antiemetics | HTR2A |
| THIETHYLPERAZINE | Antiemetics | HTR2C |
| THIOPENTAL | Anesthetics, Intravenous | CHRNA4 |
| THIOPENTAL | Anesthetics, Intravenous | CHRNA7 |
| THIOPENTAL | Anesthetics, Intravenous | GABRA1 |
| THIOPENTAL | Anesthetics, Intravenous | GABRA2 |
| THIOPENTAL | Anesthetics, Intravenous | GABRA3 |
| THIOPENTAL | Anesthetics, Intravenous | GABRA4 |
| THIOPENTAL | Anesthetics, Intravenous | GABRA5 |
| THIOPENTAL | Anesthetics, Intravenous | GABRA6 |
| THIOPENTAL | Anesthetics, Intravenous | GRIA2 |
| THIOPENTAL | Anesthetics, Intravenous | GRIK2 |
| THIORIDAZINE | Antipsychotic Agents | ADRA1A |
| THIORIDAZINE | Antipsychotic Agents | DRD1 |
| THIORIDAZINE | Antipsychotic Agents | DRD2 |
| THIORIDAZINE | Antipsychotic Agents | HTR2A |
| TIAGABINE | Anticonvulsants | ABAT |
| TIAGABINE | Anticonvulsants | SLC6A1 |
| TIAPROFENIC ACID | NSAID | PTGS2 |
| TICLOPIDINE | Platelet Aggregation Inhibitors | P2RY12 |
| TILUDRONATE | Bisphosphonates | PTPN1 |
| TIROFIBAN | Platelet Aggregation Inhibitors | ITGA2B |
| TIROFIBAN | Platelet Aggregation Inhibitors | ITGB3 |
| TOCAINIDE | Anti-Arrhythmia Agents | SCN5A |
| TOLAZAMIDE | Hypoglycemic Agents | KCNJ1 |
| TOLAZOLINE | Antihypertensive Agents | ADRA1A |
| TOLBUTAMIDE | Hypoglycemic Agents | KCNJ1 |
| TOLCAPONE | Antiparkinson Agents | COMT |
| TOLMETIN | NSAID | PTGS1 |
| TOLMETIN | NSAID | PTGS2 |
| TOPIRAMATE | Anticonvulsants; anti-migraine agents | CA2 |
| TOPIRAMATE | Anticonvulsants; anti-migraine agents | CA4 |
| TOPIRAMATE | Anticonvulsants; anti-migraine agents | GABRA1 |
| TOPIRAMATE | Anticonvulsants; anti-migraine agents | GRIK1 |
| TOPIRAMATE | Anticonvulsants; anti-migraine agents | SCN1A |
| TORASEMIDE | Antihypertensive Agents; Diuretics | SLC12A1 |
| TRANYLCYPROMINE | Antidepressive Agents | MAOA |
| TRANYLCYPROMINE | Antidepressive Agents | MAOB |
| TREPROSTINIL | Antihypertensive Agents; Antithrombotic | P2RY12 |
| | Agents | |
| TREPROSTINIL | Antihypertensive Agents; Antithrombotic | PPARG |
| | Agents | |
| TRIAMTERENE | Antihypertensive Agents; Diuretics | SCNN1A |
| TRIAMTERENE | Antihypertensive Agents; Diuretics | SCNN1B |
| TRIAMTERENE | Antihypertensive Agents; Diuretics | SCNN1D |
| TRIAMTERENE | Antihypertensive Agents; Diuretics | SCNN1G |
| TRICHLORMETHIAZIDE | Antihypertensive Agents; Diuretics | CA1 |
| TRICHLORMETHIAZIDE | Antihypertensive Agents; Diuretics | CA2 |
| TRICHLORMETHIAZIDE | Antihypertensive Agents; Diuretics | CA4 |
| TRICHLORMETHIAZIDE | Antihypertensive Agents; Diuretics | KCNMA1 |
| TRICHLORMETHIAZIDE | Antihypertensive Agents; Diuretics | SLC12A1 |
| TRIDIHEXETHYL | GI Anti-Ulcer Agents, anticholinergic; | CHRM1 |
| | Antispasmodics | |
| TRIDIHEXETHYL | GI Anti-Ulcer Agents, anticholinergic; | CHRM2 |
| | Antispasmodics | |
| TRIDIHEXETHYL | GI Anti-Ulcer Agents, anticholinergic; | CHRM3 |
| | Antispasmodics | |
| TRIFLUOPERAZINE | Antiemetics; Antipsychotic Agents | ADRA1A |
| TRIFLUOPERAZINE | Antiemetics; Antipsychotic Agents | DRD1IP |
| TRIFLUOPERAZINE | Antiemetics; Antipsychotic Agents | DRD2 |
| TRIFLUPROMAZINE | Antiemetics; Antipsychotic Agents | CHRM1 |
| TRIFLUPROMAZINE | Antiemetics; Antipsychotic Agents | CHRM2 |
| TRIFLUPROMAZINE | Antiemetics; Antipsychotic Agents | DRD1 |
| TRIFLUPROMAZINE | Antiemetics; Antipsychotic Agents | DRD2 |
| TRIFLUPROMAZINE | Antiemetics; Antipsychotic Agents | HTR2B |
| TRIHEXYPHENIDYL | Antiparkinson Agents | CHRM1 |
| TRILOSTANE | Antiadrenal | HSD3B1 |
| TRILOSTANE | Antiadrenal | HSD3B2 |
| TRIMEPRAZINE | Antipruritics | HRH1 |
| TRIMETHADIONE | Anticonvulsants | CACNA1G |
| TRIMETHAPHAN | Antihypertensive Agents; Vasodilator Agents | CHRNA10 |
| TRIMETREXATE | Antineoplastic Agents | DHFR |
| TRIMIPRAMINE | Antidepressive Agents, Tricyclic | SLC6A2 |
| TRIMIPRAMINE | Antidepressive Agents, Tricyclic | SLC6A4 |
| TRIPELENNAMINE | Anti-Allergic Agents | HRH1 |
| TRIPROLIDINE | Anti-Allergic Agents | HRH1 |
| TROPICAMIDE | Diagnostic Agents; Mydriatics | CHRM4 |
| TUBOCURARINE | Muscle Relaxants, Skeletal | CHRNA2 |
| VALPROIC ACID | Anticonvulsants | ABAT |
| VALRUBICIN | Antineoplastic Agents | TOP2A |
| WARFARIN | Anticoagulants | VKORC1 |
| WARFARIN | Anticoagulants | VKORC1L1 |
| VINBLASTINE | Antineoplastic Agents | TUBB2A |
| VINDESINE | Antineoplastic Agents | TUBB1 |
| XIMELAGATRAN | Anticoagulants | F2 |
| YOHIMBINE | Mydriatics; Anti-impotence Agents | ADRA2A |
| YOHIMBINE | Mydriatics; Anti-impotence Agents | ADRA2B |
| YOHIMBINE | Mydriatics; Anti-impotence Agents | ADRA2C |
| ZOPICLONE | Hypnotics and Sedatives | BZRP |
| ZOPICLONE | Hypnotics and Sedatives | GABRA1 |
| ZOPICLONE | Hypnotics and Sedatives | GABRA2 |
| ZOPICLONE | Hypnotics and Sedatives | GABRA3 |
| ZOPICLONE | Hypnotics and Sedatives | GABRA5 |
| ZUCLOPENTHIXOL | Antipsychotic Agents | DRD1 |
| ZUCLOPENTHIXOL | Antipsychotic Agents | DRD2 |
| ZUCLOPENTHIXOL | Antipsychotic Agents | DRD5 |

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

In the Schemes below, where a particular protecting group, leaving group, or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5th Edition, John Wiley & Sons, 2001, Comprehensive Organic Transformations, R. C. Larock, 2nd Edition, John Wiley & Sons, 1999, and Protecting Groups in Organic Synthesis, T. W. Grconc and P. G. M. Wuts, 3rd edition, John Wiley & Sons, 1999, the entirety of each of which is hereby incorporated herein by reference.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3rd edition, John Wiley & Sons, 1999, the entirety of each of which is herein incorporated by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio) pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl) ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3rd edition, John Wiley & Sons, 1999, the entirety of each of which is herein incorporated by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

In certain embodiments, compounds of the present invention are generally prepared according to Scheme 1 set forth below:

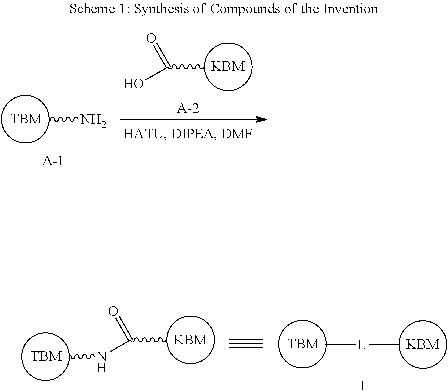

As depicted in Scheme 1, above, amine A-1 is coupled to acid A-2 using the coupling agent HATU in the presence of the base DIPEA in DMF to form a compound of the invention with a linker comprising an amide bond. The squiggly bond, [symbol] , represents the portion of the linker between TBM and the terminal amino group of A-1 or the portion of the linker between KBM and the terminal carboxyl group of A-2, respectively. Additionally, an amide bond can be formed using coupling reagents known in the art such as, but not limited to DCC, DIC, EDC, HBTU, HCTU, PyAOP, PyBrOP, BOP, BOP-CI, DEPBT, T3P, TATU, TBTU, TNTU, TOTU, TPTU, TSTU, or TDBTU.

In certain embodiments, compounds of the present invention are generally prepared according to Scheme 2 set forth below:

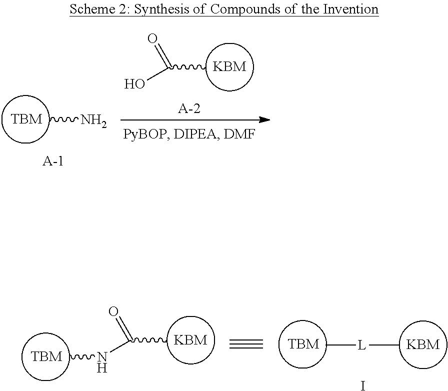

As depicted in Scheme 2, above, amine A-1 is coupled to acid A-2 using the coupling agent PyBOP in the presence of the base DIPEA in DMF to form a compound of the invention with a linker comprising an amide bond. The squiggly bond, [symbol] , represents the portion of the linker between TBM and the terminal amino group of A-1 or the portion of the linker between KBM and the terminal carboxyl group of A-2, respectively. Additionally, an amide bond can be formed using coupling reagents known in the art such as, but not limited to DCC, DIC, EDC, HBTU, HCTU, PyAOP, PyBrOP, BOP, BOP-CI, DEPBT, T3P, TATU, TBTU, TNTU, TOTU, TPTU, TSTU, or TDBTU.

In certain embodiments, compounds of the present invention are generally prepared according to Scheme 3 set forth below:

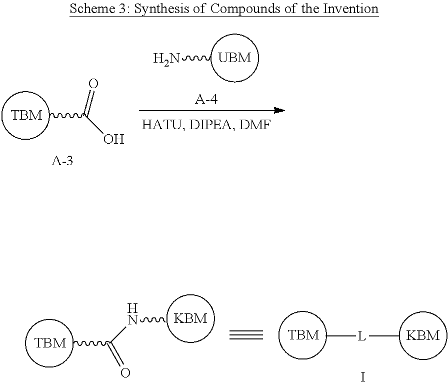

As depicted in Scheme 3, above, acid A-3 is coupled to amine A-4 using the coupling agent HATU in the presence of the base DIPEA in DMF to form a compound of the invention with a linker comprising an amide bond. The squiggly bond, [symbol] , represents the portion of the linker between TBM and the terminal carboxyl group of A-3 or the portion of the linker between KBM and the terminal amino group of A-4, respectively. Additionally, an amide bond can be formed using coupling reagents known in the art such as, but not limited to DCC, DIC, EDC, HBTU, HCTU, PyAOP, PyBrOP, BOP, BOP-CI, DEPBT, T3P, TATU, TBTU, TNTU, TOTU, TPTU, TSTU, or TDBTU.

In certain embodiments, compounds of the present invention are generally prepared according to Scheme 4 set forth below:

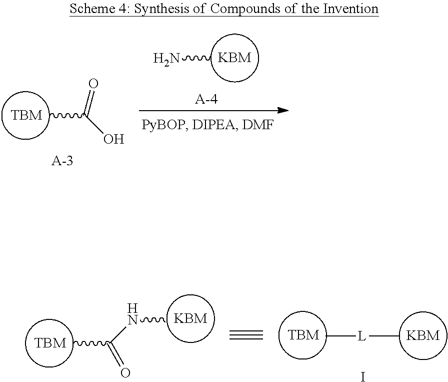

As depicted in Scheme 4, above, acid A-3 is coupled to amine A-4 using the coupling agent PyBOP in the presence of the base DIPEA in DMF to form a compound of the invention with a linker comprising an amide bond. The squiggly bond, [symbol] , represents the portion of the linker between TBM and the terminal carboxyl group of A-3 or the portion of the linker between KBM and the terminal amino group of A-4, respectively. Additionally, an amide bond can be formed using coupling reagents known in the art such as, but not limited to DCC, DIC, EDC, HBTU, HCTU, PyAOP, PyBrOP, BOP, BOP-CI, DEPBT, T3P, TATU, TBTU, TNTU, TOTU, TPTU, TSTU, or TDBTU.

In certain embodiments, compounds of the present invention are generally prepared according to Scheme 5 set forth below:

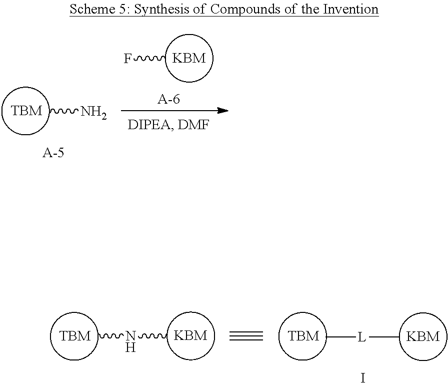

As depicted in Scheme 5, above, an SNAr displacement of fluoride A-6 by amine A-5 is effected in the presence of the base DIPEA in DMF to form a compound of the invention with a linker comprising a secondary amine. The squiggly bond, [symbol] , represents the portion of the linker between TBM and the terminal amino group of A-5.

In certain embodiments, compounds of the present invention are generally prepared according to Scheme 6 set forth below:

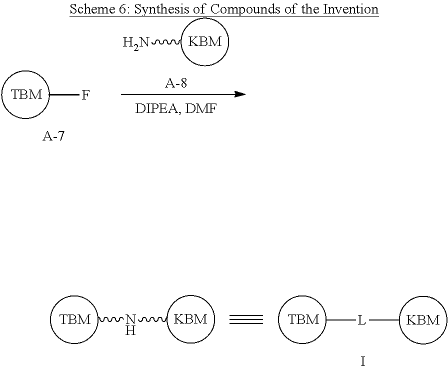

As depicted in Scheme 6, above, an SNAr displacement of fluoride A-7 by amine A-8 is effected in the presence of the base DIPEA in DMF to form a compound of the invention with a linker comprising a secondary amine. The squiggly bond, [symbol] , represents the portion of the linker between KBM and the terminal amino group of A-8.

In certain embodiments, compounds of the present invention are generally prepared according to Scheme 7 set forth below:

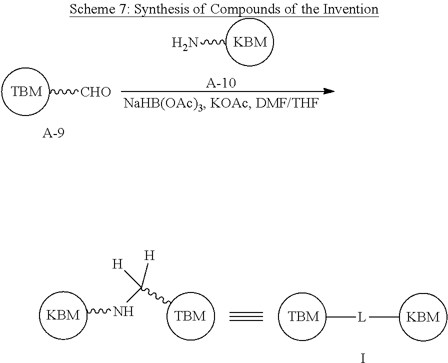

As depicted in Scheme 7, above, reductive amination of the mixture of aldehyde A-9 and amine A-10 is effected in the presence of NaHB (OAc)₃ and KOAc in DMF/THF to form a compound of the invention with a linker comprising a secondary amine. A linker comprising a tertiary amine can be prepared similarily using a secondary amine in place of the primary amine A-10. The squiggly bond, [symbol] , represents the portion of the linker between TBM and the terminal aldehyde of A-9 or the portion of the linker between KBM and the terminal amino group of A-10, respectively.

In certain embodiments, compounds of the present invention are generally prepared according to Scheme 8 set forth below:

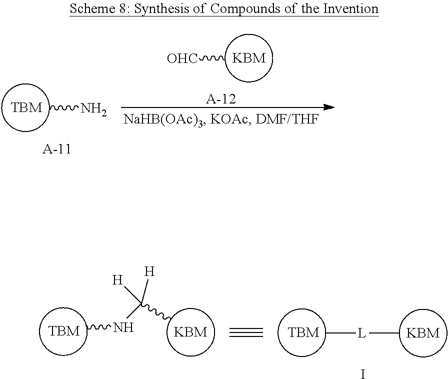

As depicted in Scheme 8, above, reductive amination of the mixture of aldchydc A-12 and amine A-11 is effected in the presence of NaHB (OAc)₃ and KOAc in DMF/THF to form a compound of the invention with a linker comprising a secondary amine. A linker comprising a tertiary amine can be prepared similarily using a secondary amine in place of the primary amine A-11. The squiggly bond, [symbol] , represents the portion of the linker between TBM and the terminal amino group of A-11 or the portion of the linker between KBM and the terminal aldehyde of A-12, respectively.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. See for example, “March's Advanced Organic Chemistry”, 5th Ed., Ed.: Smith, M. B. and March, J., John Wiley & Sons, New York: 2001, the entirety of each of which is herein incorporated by reference. Such interconversions may require one or more of the aforementioned techniques, and certain methods for synthesizing compounds of the invention are described below in the Exemplification.

### 5. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably bind KLHDC2, or a mutant thereof, and a targeted protein, or a mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

As used herein, the term “active metabolite or residue thereof” means that a metabolite or residue thereof is also a binder of KLHDC2, or a mutant thereof, or a targeted protein, or a mutant thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

Presently described are compositions and methods that relate to the discovery that an E3 ubiquitin ligase protein (e.g., KLHDC2) ubiquitinates a target protein once it and the target protein are placed in proximity by a bifunctional or chimeric construct that binds the E3 ubiquitin ligase protein (e.g., KLHDC2) and the target protein. Accordingly the present invention provides such compounds and compositions comprising an KLHDC2 E3 ubiquintin ligase binding moiety (“KBM”) coupled to a protein target binding moiety (“TBM”), which result in the ubiquitination of a chosen target protein, which leads to degradation of the target protein by the proteasome.

Compounds and compositions described herein are generally useful for the modulation of targeted ubiquitination, especially with respect to a variety of polypeptides and other proteins, which are degraded and/or otherwise inhibited. Compounds and compositions described herein exhibit a broad range of pharmacological activities, consistent with the degradation/inhibition of targeted polypeptides.

Even though KLHDC2 binders are known in the art, there is a continuing need to provide novel binders having more effective or advantageous pharmaceutically relevant properties. For example, compounds with increased activity, selectivity over other E3 ligases, and ADMET (absorption, distribution, metabolism, excretion, and/or toxicity) properties. Thus, in some embodiments, the present invention provides binders of KLHDC2 which show selectivity over other E3 ligases. Such compounds should deliver a pharmacological response that favorably treats one or more of the conditions described herein without the side-effects associated with the binding of E3 ligases.

The activity of a compound utilized in this invention as an binder of KLHDC2, or a mutant thereof, may be assayed in vitro, in vivo or in a cell line. In vitro assays include assays that determine the subsequent functional consequences, or activity of activated KLHDC2, or a mutant thereof. Alternate in vitro assays quantitate the ability of the compound to bind to KLHDC2. Binding may be measured by radiolabeling the compound prior to binding, isolating the compound/KLHDC2 complex and determining the amount of radiolabel bound. Alternatively, compound binding may be determined by running a competition experiment where new compounds are incubated with KLHDC2 bound to known radioligands.

The term “ubiquitin ligase” refers to a family of proteins that facilitate the transfer of ubiquitin to a specific substrate protein, targeting the substrate protein for degradation. For example, cereblon is an E3 ubiquitin ligase protein that alone or in combination with an E2 ubiquitin-conjugating enzyme causes the attachment of ubiquitin to a lysine on a target protein, and subsequently targets the specific protein substrates for degradation by the proteasome. Thus, E3 ubiquitin ligase alone or in complex with an E2 ubiquitin conjugating enzyme is responsible for the transfer of ubiquitin to targeted proteins. In general, the ubiquitin ligase is involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation, but may instead be altered in their cellular location or function, for example, via binding other proteins that have domains capable of binding ubiquitin. Further complicating matters, different lysines on ubiquitin can be targeted by an E3 to make chains. Accordingly in some embodiments, a provided compound specifically recognizes proteins with a diglycine (Gly-Gly) at the C-terminus, leading to their ubiquitination and degradation.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The description provides therapeutic compositions as described herein for effectuating the degradation of proteins of interest for the treatment or amelioration of a disease, e.g., cancer. As such, in another aspect, the description provides a method of ubiquitinating/degrading a target protein in a cell. In certain embodiments, the method comprises administering a bifunctional compound as described herein comprising, e.g., a KBM and a TBM, linked through a linker moiety, as otherwise described herein, wherein the KBM is coupled to the TBM and wherein the KBM recognizes a ubiquitin pathway protein (e.g., an ubiquitin ligase, preferably an E3 ubiquitin ligase such as, e.g., KLHDC2) and the TBM recognizes the target protein such that degradation of the target protein will occur when the target protein is placed in proximity to the ubiquitin ligase, thus resulting in degradation/inhibition of the effects of the target protein and control of protein levels. The control of protein levels afforded by the present invention provides treatment of a disease state or condition, which is modulated through the target protein by lowering the level of that protein in the cell, e.g., cell of a patient. In certain embodiments, the method comprises administering an effective amount of a compound as described herein, optionally including a pharmaceutically acceptable excipient, carrier, adjuvant, another bioactive agent or combination thereof.

In additional embodiments, the description provides methods for treating or emeliorating a disease, disorder or symptom thereof in a subject or a patient, comprising administering to a subject in need thereof a composition comprising an effective amount, e.g., a therapeutically effective amount, of a compound as described herein or salt form thereof, and a pharmaceutically acceptable excipient, carrier, adjuvant, another bioactive agent or combination thereof, wherein the composition is effective for treating or ameliorating the disease or disorder or symptom thereof in the subject.

In another aspect, the description provides methods for identifying the effects of the degradation of proteins of interest in a biological system using compounds according to the present invention.

In another embodiment, the present invention is directed to a method of treating a human patient in need for a disease state or condition modulated through a protein where the degradation of that protein will produce a therapeutic effect in that patient, the method comprising administering to a patient in need an effective amount of a compound according to the present invention, optionally in combination with another bioactive agent. The disease state or condition may be a disease caused by a microbial agent or other exogenous agent such as a virus, bacteria, fungus, protozoa or other microbe or may be a disease state, which is caused by overexpression of a protein, which leads to a disease state and/or condition.

According to one embodiment, the invention relates to a method of modulating KLHDC2 activity in a biological sample comprising the step of contacting said biological sample with a compound of this invention, or a composition comprising said compound.

According to another embodiment, the invention relates to a method of binding KLHDC2, or a mutant thereof, activity in a biological sample comprising the step of contacting said biological sample with a compound of this invention, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Binding KLHDC2 (or a mutant thereof) activity in a biological sample is useful for a variety of purposes that are known to one of skill in the art. Examples of such purposes include, but are not limited to, biological specimen storage and biological assays.

Another embodiment of the present invention relates to a method of modulating KLHDC2 activity in a patient comprising the step of administering to said patient a compound of the present invention, or a composition comprising said compound.

According to another embodiment, the invention relates to a method of modulating the activity of KLHDC2, or a mutant thereof, in a patient comprising the step of administering to said patient a compound of the present invention, or a composition comprising said compound. According to certain embodiments, the invention relates to a method of reversibly or irreversibly modulating one or more of KLHDC2, or a mutant thereof, activity in a patient comprising the step of administering to said patient a compound of the present invention, or a composition comprising said compound. In other embodiments, the present invention provides a method for treating a disorder mediated by KLHDC2, or a mutant thereof, in a patient in need thereof, comprising the step of administering to said patient a compound according to the present invention or pharmaceutically acceptable composition thereof.

In some embodiments, a provided compound according to the present invention is used in the treatment of a disease state or condition, for example, asthma, autoimmune diseases such as multiple sclerosis, various cancers, ciliopathies, cleft palate, diabetes, heart disease, hypertension, inflammatory bowel disease, mental retardation, mood disorder, obesity, refractive error, infertility, Angelman syndrome, Canavan disease, Coeliac disease, Charcot-Marie-Tooth disease, Cystic fibrosis, Duchenne muscular dystrophy, Haemochromatosis, Haemophilia, Klinefelter's syndrome, Neurofibromatosis, Phenylketonuria, Polycystic kidney disease, (PKD1) or 4 (PKD2) Prader-Willi syndrome, Sickle-cell disease, Tay-Sachs disease, or Turner syndrome.

Further disease states or conditions which may be treated by compounds according to the present invention include Alzheimer's disease, amyotrophic lateral sclerosis (Lou Gehrig's disease), anorexia nervosa, anxiety disorder, atherosclerosis, attention deficit hyperactivity disorder, autism, bipolar disorder, chronic fatigue syndrome, chronic obstructive pulmonary disease, Crohn's disease, coronary heart disease, dementia, depression, diabetes mellitus type 1, diabetes mellitus type 2, epilepsy, Guillain-Barré syndrome, irritable bowel syndrome, lupus, metabolic syndrome, multiple sclerosis, myocardial infarction, obesity, obsessive compulsive disorder, panic disorder, Parkinson's disease, psoriasis, rheumatoid arthritis, sarcoidosis, schizophrenia, stroke, thromboangiitis obliterans, Tourette syndrome, and vasculitis.

Still additional disease states or conditions which can be treated by compounds according to the present invention include aceruloplasminemia, achondrogenesis type II, achondroplasia, acrocephaly, Gaucher disease type 2, acute intermittent porphyria, canavan disease, adenomatous polyposis coli, ALA dehydratase deficiency, adenylosuccinate deficiency, lyase Adrenogenital syndrome, adrenoleukodystrophy, ALA-D porphyria, ALA dehydratase deficiency, alkaptonuria, Alexander disease, alkaptonuric ochronosis, alpha 1-antitrypsin deficiency, alpha-1 proteinase inhibitor, emphysema, amyotrophic lateral sclerosis Alström syndrome, Alexander disease, amelogenesis imperfecta, ALA dehydratase deficiency, Anderson-Fabry disease, androgen insensitivity syndrome, anemia angiokeratoma corporis diffusum, angiomatosis retinae (von Hippel-Lindau disease) Apert syndrome, arachnodactyly (Marfan syndrome), Stickler syndrome, arthrochalasis multiplex congenital (Ehlers-Danlos syndrome #arthrochalasia type) ataxia telangiectasia, Rett syndrome, primary pulmonary hypertension, Sandhoff disease, neurofibromatosis type II, Beare —Stevenson cutis gyrata syndrome, Mediterranean fever, familial, Benjamin syndrome, beta-thalassemia, bilateral acoustic neurofibromatosis (neurofibromatosis type II), factor V Leiden thrombophilia, Bloch-Sulzberger syndrome (incontinentia pigmenti), Bloom syndrome, X-linked sideroblastic anemia, Bonnevie-Ullrich syndrome (Turner syndrome), Bourneville disease (tuberous sclerosis), prion disease, Birt-Hogg-Dubé syndrome, Brittle bone disease (osteogenesis imperfecta), Broad Thumb-Hallux syndromc (Rubinstein-Taybi syndromc), Bronze Diabetes/Bronzed Cirrhosis (hemochromatosis), Bulbospinal muscular atrophy (Kennedy's disease), Burger-Grutz syndrome (lipoprotein lipase deficiency), CGD Chronic granulomatous disorder, Campomelic dysplasia, biotinidase deficiency, cardiomyopathy (Noonan syndrome), Cri du chat, CAVD (congenital absence of the vas deferens), Caylor cardiofacial syndrome (CBAVD), CEP(congenital erythropoietic porphyria), cystic fibrosis, congenital hypothyroidism, chondrodystrophy syndrome (achondroplasia), otospondylomegaepiphyseal dysplasia, Lesch-Nyhan syndrome, galactosemia, Ehlers-Danlos syndrome, Thanatophoric dysplasia, Coffin-Lowry syndrome, Cockayne syndrome, (familial adenomatous polyposis), Congenital erythropoietic porphyria, congenital heart disease, Methemoglobinemia/Congenital methaemoglobinaemia, achondroplasia, X-linked sideroblastic anemia, connective tissue disease, Conotruncal anomaly face syndrome, Cooley's Anemia (beta-thalassemia), copper storage disease (Wilson's disease), copper transport disease (Menkes disease), hereditary coproporphyria, Cowden syndrome, craniofacial dysarthrosis (Crouzon syndrome), Creutzfeldt-Jakob disease (prion disease), Cockayne syndrome, Cowden syndrome, Curschmann-Batten-Steinert syndrome (myotonic dystrophy), Beare —Stevenson cutis gyrata syndrome, primary hyperoxaluria, spondyloepimetaphyseal dysplasia (Strudwick type), muscular dystrophy, Duchenne and Becker types (DBMD), Usher syndrome, Degenerative nerve diseases including de Grouchy syndrome and Dejerine-Sottas syndrome, developmental disabilities, distal spinal muscular atrophy, type V, androgen insensitivity syndrome, Diffuse Globoid Body Sclerosis (Krabbe disease), Di George's syndrome, Dihydrotestosterone receptor deficiency, androgen insensitivity syndrome, Down syndrome, Dwarfism, erythropoietic protoporphyria erythroid 5-aminolevulinate synthetase deficiency, erythropoietic porphyria, erythropoietic protoporphyria, erythropoietic uroporphyria, Friedreich's ataxia, familial paroxysmal polyserositis, porphyria cutanea tarda, familial pressure sensitive neuropathy, primary pulmonary hypertension (PPH), Fibrocystic disease of the pancreas, fragile X syndrome, galactosemia, genetic brain disorders, giant cell hepatitis (Neonatal hemochromatosis), Gronblad-Strandberg syndrome (pseudoxanthoma elasticum), Gunther disease (congenital erythropoietic porphyria), haemochromatosis, Hallgren syndrome, sickle cell anemia, hemophilia, hepatoerythropoietic porphyria (HEP), Hippel-Lindau disease (von Hippel-Lindau disease), Huntington's disease, Hutchinson-Gilford progeria syndrome (progeria), Hyperandrogenism, Hypochondroplasia, Hypochromic anemia, Immune system disorders, including X-linked severe combined immunodeficiency, Insley-Astley syndrome, Jackson-Weiss syndrome, Joubert syndrome, Lesch-Nyhan syndrome, Jackson-Weiss syndrome, kidney diseases, including hyperoxaluria, Klinefelter's syndrome, Kniest dysplasia, Lacunar dementia, Langer-Saldino achondrogenesis, ataxia telangiectasia, Lynch syndrome, Lysyl-hydroxylase deficiency, Machado-Joseph disease, Metabolic disorders, including Kniest dysplasia, Marfan syndrome, movement disorders, Mowat-Wilson syndrome, cystic fibrosis, Muenke syndromc, Multiple neurofibromatosis, Nancc-Insley syndromc, Nancc-Swcency chondrodysplasia, Niemann-Pick disease, Noack syndrome (Pfeiffer syndrome), Osler-Weber-Rendu disease, Peutz-Jeghers syndrome, Polycystic kidney disease, polyostotic fibrous dysplasia (McCune-Albright syndrome), Peutz-Jeghers syndrome, Prader-Labhart-Willi syndrome, hemochromatosis, primary hyperuricemia syndrome (Lesch-Nyhan syndrome), primary pulmonary hypertension, primary senile degenerative dementia, prion disease, progeria (Hutchinson Gilford Progeria Syndrome), progressive chorea, chronic hereditary (Huntington)(Huntington's disease), progressive muscular atrophy, spinal muscular atrophy, propionic acidemia, protoporphyria, proximal myotonic dystrophy, pulmonary arterial hypertension, PXE (pseudoxanthoma elasticum), Rb (retinoblastoma), Recklinghausen disease (neurofibromatosis type I), Recurrent polyserositis, Retinal disorders, Retinoblastoma, Rett syndrome, RFALS type 3, Ricker syndrome, Riley-Day syndrome, Roussy-Levy syndrome, severe achondroplasia with developmental delay and acanthosis nigricans (SADDAN), Li-Fraumeni syndrome, sarcoma, breast, leukemia, and adrenal gland (SBLA) syndrome, sclerosis tuberose (tuberous sclerosis), SDAT, SED congenital (spondyloepiphyseal dysplasia congenita), SED Strudwick (spondyloepimetaphyseal dysplasia, Strudwick type), SEDc (spondyloepiphyseal dysplasia congenita) SEMD, Strudwick type (spondyloepimetaphyseal dysplasia, Strudwick type), Shprintzen syndrome, Skin pigmentation disorders, Smith-Lemli-Opitz syndrome, South-African genetic porphyria (variegate porphyria), infantile-onset ascending hereditary spastic paralysis, Speech and communication disorders, sphingolipidosis, Tay-Sachs disease, spinocerebellar ataxia, Stickler syndrome, stroke, androgen insensitivity syndrome, tetrahydrobiopterin deficiency, beta-thalassemia, thyroid disease, Tomaculous neuropathy (hereditary neuropathy with liability to pressure palsies), Treacher Collins syndrome, Triplo X syndrome (triple X syndrome), Trisomy 21 (Down syndrome), Trisomy X, VHL syndrome (von Hippel-Lindau disease), Vision impairment and blindness (Alström syndrome), Vrolik disease, Waardenburg syndrome, Warburg Sjo Fledelius Syndrome, Weissenbacher-Zweymüller syndrome, Wolf-Hirschhorn syndrome, Wolff Periodic disease, Weissenbacher-Zweymüller syndrome and Xeroderma pigmentosum, among others.

The term “neoplasia” or “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Exemplary cancers which may be treated by the present compounds cither alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present invention include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

In some embodiments, the present invention provides a method for treating one or more disorders, wherein the disorders are selected from autoimmune disorders, inflammatory disorders, proliferative disorders, endocrine disorders, neurological disorders, and disorders associated with transplantation, said method comprising administering to a patient in need thereof, a pharmaceutical composition comprising an effective amount of a compound of the present invention, or a pharmaceutically acceptable salt thereof.

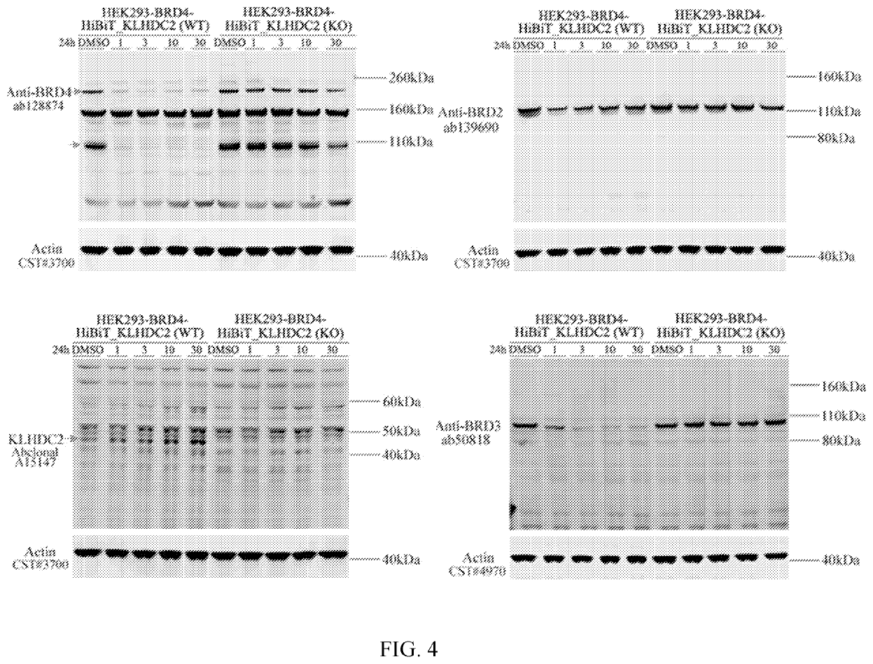

In some embodiments, compounds of the present invention induce the ubiquitination and degradation of a target protein selected from the group consisting of A1BG, A1CF, A2M, A2ML1, A3GALT2, A4GALT, A4GNT, AAAS, AACS, AADAC, AADACL2, AADACL3, AADACL4, AADAT, AAEDI, AAGAB, AAKI, AAMDC, AAMP, AANAT, AAR2, AARD, AARS, AARS2, AARSDI, AASDH, AASDHPPT, AASS, AATF, AATK, AATK-ASI, ABAT, ABCAI, ABCA10, ABCA12, ABCA13, ABCA2, ABCA3, ABCA4, ABCA5, ABCA6, ABCA7, ABCA8, ABCA9, ABCBI, ABCB10, ABCB11, ABCB4, ABCB5, ABCB6, ABCB7, ABCB8, ABCB9, ABCC1, ABCC10, ABCC11, ABCC12, ABCC2, ABCC3, ABCC4, ABCC5, ABCC6, ABCC8, ABCC9, ABCD1, ABCD2, ABCD3, ABCD4, ABCE1, ABCF1, ABCF2, ABCF3, ABCG1, ABCG2, ABCG4, ABCG5, ABCG8, ABHD1, ABHD10, ABHD11, ABHD12, ABHD12B, ABHD13, ABHD14A, ABHD14A-ACY1, ABHD14B, ABHD15, ABHD16A, ABHD16B, ABHD17A, ABHD17B, ABHD17C, ABHD18, ABHD2, ABHD3, ABHD4, ABHD5, ABHD6, ABHD8, ABI1, ABI2, ABI3, ABI3BP, ABL1, ABL2, ABLIM1, ABLIM2, ABLIM3, ABO, ABR, ABRA, ABRACL, ABRAXAS1, ABRAXAS2, ABTI, ABTB1, ABTB2, AC001226.2, AC002094.3, AC002115.2, AC002310.4, AC002310.5, AC002429.2, AC002985.1, AC002996.1, AC003002.1, AC003002.2, AC003002.3, AC003002.4, AC003005.1, AC003006.1, AC003688.1, AC004076.1, AC004080.3, AC004223.3, AC004233.2, AC004556.1, AC004691.2, AC004706.4, AC004754.1, AC004805.1, AC004832.3, AC004922.1, AC004997.1, AC005020.2, AC005041.1, AC005154.6, AC005258.1, AC005324.3, AC005324.4, AC005520.1, AC005551.1, AC005670.2, AC005697.1, AC005702.2, AC005726.2, AC005779.2, AC005832.4, AC005833.1, AC005833.3, AC005837.2, AC005841.2, AC005885.1, AC005943.1, AC006030.1, AC006254.1, AC006269.1, AC006449.4, AC006486.1, AC006538.2, AC006978.2, AC007040.2, AC007192.1, AC007240.1, AC007325.1, AC007325.2, AC007325.4, AC007326.4, AC007375.2, AC007383.6, AC007537.5, AC007731.5, AC007906.2, AC007998.2, AC008073.3, AC008162.2, AC008393.2, AC008403.1, AC008481.3, AC008537.1, AC008560.1, AC008575.1, AC008575.2, AC008687.1, AC008687.4, AC008687.8, AC008695.1, AC008735.6, AC008750.8, AC008758.1, AC008758.4, AC008758.5, AC008758.6, AC008763.2, AC008763.3, AC008764.1, AC008764.4, AC008770.2, AC008770.3, AC008878.1, AC008878.2, AC008878.3, AC008982.1, AC008982.3, AC009014.1, AC009086.2, AC009119.2, AC009122.1, AC009133.6, AC009163.2, AC009163.4, AC009286.3, AC009336.2, AC009477.2, AC009690.1, AC009690.3, AC009779.3, AC010132.3, AC010255.3, AC010319.2, AC010323.1, AC010325.1, AC010326.2, AC010327.1, AC010422.3, AC010422.5, AC010422.6, AC010463.1, AC010487.3, AC010522.1, AC010531.1, AC010542.3, AC010547.4, AC010547.5, AC010615.4, AC010616.1, AC010619.1, AC010646.1, AC010724.2, AC011005.1, AC011043.1, AC011043.2, AC011195.2, AC011295.1, AC011346.1, AC011448.1, AC011452.1, AC011455.3, AC011455.4, AC011462.1, AC011473.4, AC011479.1, AC011498.4, AC011499.1, AC011511.1, AC011511.4, AC011530.1, AC011604.2, AC011841.1, AC012184.2, AC012254.2, AC012309.1, AC012314.1, AC012314.10, AC012314.11, AC012314.12, AC012314.4, AC012314.5, AC012314.6, AC012314.8, AC012531.3, AC012651.1, AC013269.1, AC013271.1, AC013394.1, AC013470.2, AC015688.5, AC015802.6, AC015813.2, AC017081.3, AC017081.4, AC017081.5, AC017083.4, AC018512.1, AC018523.2, AC018554.3, AC018630.6, AC018709.1, AC018755.2, AC018793.1, AC018793.2, AC018793.3, AC018793.4, AC018793.5, AC019117.3, AC020636.2, AC020909.1, AC020914.1, AC020915.1, AC020915.2, AC020915.6, AC020922.1, AC020934.3, AC021072.1, AC022016.2, AC022167.5, AC022335.1, AC022384.1, AC022400.6, AC022826.2, AC023055.1, AC023491.2, AC023509.3, AC024592.3, AC024940.1, AC024940.6, AC025165.3, AC025263.2, AC025283.2, AC025287.4, AC025594.2, AC026369.8, AC026398.1, AC026461.4, AC026464.1, AC026464.3, AC026464.4, AC026786.1, AC026954.2, AC027796.3, AC034102.2, AC036214.3, AC037459.1, AC037482.2, AC037482.3, AC040162.1, AC040162.4, AC044810.8, AC046185.1, AC048338.1, AC051649.2, AC053481.5, AC055811.2, AC058822.1, AC064853.2, AC064853.3, AC064853.4, AC064853.5, AC064853.6, AC067968.1, AC068234.1, AC068533.4, AC068547.1, AC068580.4, AC068631.2, AC068775.1, AC068775.2, AC068790.8, AC068896.1, AC068946.1, AC068987.5, AC069257.3, AC069368.1, AC069503.2, AC069544.2, AC072022.1, AC073082.1, AC073111.3, AC073111.5, AC073264.3, AC073508.2, AC073610.2, AC073610.3, AC073612.1, AC073896.1, AC074143.1, AC078927.1, AC079325.2, AC079447.1, AC079594.2, AC083800.1, AC083902.2, AC084337.2, AC087289.3, AC087498.1, AC087632.1, AC090004.1, AC090227.1, AC090360.1, AC090527.2, AC090958.3, AC091167.3, AC091167.7, AC091167.8, AC091304.7, AC091491.1, AC091551.1, AC091959.3, AC091980.2, AC092017.3, AC092042.3, AC092073.1, AC092111.3, AC092143.1, AC092329.3, AC092442.1, AC092587.1, AC092647.5, AC092718.3, AC092718.8, AC092821.1, AC092824.3, AC092835.1, AC093155.3, AC093227.3, AC093423.3, AC093525.1, AC093525.2, AC093668.1, AC093762.1, AC093762.2, AC093762.3, AC093899.2, AC096582.3, AC096887.1, AC097372.1, AC097495.1, AC097637.1, AC097662.2, AC098484.3, AC098650.1, AC098850.4, AC099329.3, AC099489.1, AC099518.3, AC099811.2, AC099850.2, AC100868.1, AC104109.3, AC104151.1, AC104304.1, AC104452.1, AC104532.1, AC104534.3, AC104581.1, AC104581.3, AC104662.2, AC104836.1, AC105001.2, AC105052.1, AC106774.10, AC106774.5, AC106774.6, AC106774.7, AC106774.8, AC106774.9, AC106782.1, AC106886.5, AC107871.1, AC108488.2, AC108750.1, AC108941.2, AC109583.3, AC110275.1, AC112229.3, AC112484.1, AC113189.6, AC113189.9, AC113331.2, AC113554.2, AC114296.1, AC114490.2, AC115220.1, AC116366.3, AC116565.1, AC117457.1, AC118470.1, AC118553.2, AC119396.1, AC119674.2, AC120057.3, AC120114.5, AC124312.1, AC126755.2, AC127537.5, AC127537.6, AC127537.8, AC129492.3, AC131097.2, AC131160.1, AC133551.1, AC133555.3, AC134669.2, AC134772.2, AC135050.2, AC135068.1, AC135068.2, AC135068.3, AC135068.8, AC135178.2, AC135586.2, AC136352.3, AC136352.4, AC136428.1, AC136612.1, AC136616.1, AC136616.2, AC136616.3, AC137834.1, AC138517.2, AC138647.1, AC138696.1, AC138811.2, AC138894.1, AC138969.1, AC139530.2, AC139677.1, AC139677.2, AC140504.1, AC141272.1, AC142391.1, AC142525.4, AC145029.2, AC145212.1, AC145212.2, AC171558.1, AC171558.3, AC171558.5, AC171558.6, AC187653.1, AC207056.1, AC209232.1, AC209539.2, AC210544.1, AC213203.1, AC229888.1, AC229888.10, AC229888.2, AC229888.3, AC229888.4, AC229888.5, AC229888.6, AC229888.7, AC229888.8, AC229888.9, AC233282.1, AC233282.2, AC233723.1, AC233724.12, AC233724.16, AC233724.17, AC233724.18, AC233724.19, AC233724.20, AC233724.21, AC233724.6, AC233755.1, AC233755.2, AC233992.2, AC234301.1, AC234301.3, AC234635.1, AC234635.3, AC234635.4, AC234635.5, AC236040.1, AC239612.1, AC239618.1, AC239618.2, AC239618.3, AC239618.4, AC239618.5, AC239618.6, AC239618.7, AC239618.9, AC239799.1, AC240274.1, AC241401.1, AC241409.2, AC241410.1, AC241556.3, AC241556.4, AC241640.1, AC241640.2, AC241640.4, AC242528.1, AC242528.2, AC243547.3, AC243733.1, AC243734.1, AC243756.1, AC243790.1, AC243967.1, AC244196.1, AC244196.2, AC244196.3, AC244196.4, AC244196.5, AC244197.3, AC244216.4, AC244216.5, AC244226.1, AC244226.2, AC244472.1, AC244472.2, AC244472.3, AC244472.4, AC244472.5, AC244489.1, AC244489.2, AC244517.10, AC244517.6, AC245033.1, AC245034.2, AC245078.1, AC245088.2, AC245088.3, AC245369.1, AC245369.2, AC245369.3, AC245369.4, AC245369.6, AC245427.1, AC245427.3, AC245427.4, AC245427.5, AC245427.6, AC245427.7, AC245427.8, AC245427.9, AC245748.1, AC247036.3, AC247036.4, AC247036.5, AC247036.6, AC254560.1, AC254788.1, AC254788.2, AC254952.1, AC255093.3, AC255093.5, AC256236.1, AC256236.2, AC256236.3, AC256300.2, AC256309.2, AC270107.1, AC270107.10, AC270107.12, AC270107.2, AC270107.3, AC270107.4, AC270107.5, AC270107.7, AC270107.8, AC270107.9, AC270227.1, AC270306.4, AC275455.2, ACAA1, ACAA2, ACACA, ACACB, ACAD10, ACAD11, ACAD8, ACAD9, ACADL, ACADM, ACADS, ACADSB, ACADVL, ACAN, ACAP1, ACAP2, ACAP3, ACAT1, ACAT2, ACBD3, ACBD4, ACBD5, ACBD6, ACBD7, ACCS, ACCSL, ACD, ACE, ACE2, ACER1, ACER2, ACER3, ACHE, ACINI, ACKR1, ACKR2, ACKR3, ACKR4, ACLY, ACMSD, ACO1, ACO2, ACODI, ACOTI, ACOT11, ACOT12, ACOT13, ACOT2, ACOT4, ACOT6, ACOT7, ACOT8, ACOT9, ACOXI, ACOX2, ACOX3, ACOXL, ACPI, ACP2, ACP4, ACP5, ACP6, ACP7, ACPP, ACR, ACRBP, ACRVI, ACSBG1, ACSBG2, ACSF2, ACSF3, ACSLI, ACSL3, ACSL4, ACSL5, ACSL6, ACSMI, ACSM2A, ACSM2B, ACSM3, ACSM4, ACSM5, ACSM6, ACSS1, ACSS2, ACSS3, ACTA1, ACTA2, ACTB, ACTBL2, ACTCI, ACTGI, ACTG2, ACTL10, ACTL6A, ACTL6B, ACTL7A, ACTL7B, ACTL8, ACTL9, ACTNI, ACTN2, ACTN3, ACTN4, ACTRI0, ACTRIA, ACTRIB, ACTR2, ACTR3, ACTR3B, ACTR3C, ACTR5, ACTR6, ACTR8, ACTRTI, ACTR^{T2}, ACTR^{T3}, ACVR1, ACVRIB, ACVRIC, ACVR2A, ACVR2B, ACVRLI, ACY1, ACY3, ACYP1, ACYP2, AD000671.1, AD000671.2, ADA, ADA2, ADADI, ADAD2, ADAL, ADAM10, ADAM11, ADAM12, ADAM15, ADAM17, ADAM18, ADAM19, ADAM2, ADAM20, ADAM21, ADAM22, ADAM23, ADAM28, ADAM29, ADAM30, ADAM32, ADAM33, ADAM7, ADAM8, ADAM9, ADAMDECI, ADAMTS1, ADAMTS10, ADAMTS12, ADAMTS13, ADAMTS14, ADAMTS15, ADAMTS16, ADAMTS17, ADAMTS18, ADAMTS19, ADAMTS2, ADAMTS20, ADAMTS3, ADAMTS4, ADAMTS5, ADAMTS6, ADAMTS7, ADAMTS8, ADAMTS9, ADAMTSLI, ADAMTSL2, ADAMTSL3, ADAMTSL4, ADAMTSL5, ADAP1, ADAP2, ADAR, ADARBI, ADARB2, ADATI, ADAT2, ADAT3, ADCKI, ADCK2, ADCK5, ADCY1, ADCY10, ADCY2, ADCY3, ADCY4, ADCY5, ADCY6, ADCY7, ADCY8, ADCY9, ADCYAP1, ADCYAPIR1, ADD1, ADD2, ADD3, ADGB, ADGRA1, ADGRA2, ADGRA3, ADGRB1, ADGRB2, ADGRB3, ADGRD1, ADGRD2, ADGREI, ADGRE2, ADGRE3, ADGRE5, ADGRF1, ADGRF2, ADGRF3, ADGRF4, ADGRF5, ADGRG1, ADGRG2, ADGRG3, ADGRG4, ADGRG5, ADGRG6, ADGRG7, ADGRLI, ADGRL2, ADGRL3, ADGRL4, ADGRV1, ADHIA, ADHIB, ADHIC, ADH4, ADH5, ADH6, ADH7, ADHFE1, ADI1, ADIG, ADIPOQ, ADIPORI, ADIPOR2, ADIRF, ADK, ADM, ADM2, ADM5, ADNP, ADNP2, ADO, ADORA1, ADORA2A, ADORA2B, ADORA3, ADPGK, ADPRH, ADPRHL1, ADPRHL2, ADPRM, ADRAIA, ADRAIB, ADRAID, ADRA2A, ADRA2B, ADRA2C, ADRB1, ADRB2, ADRB3, ADRMI, ADSL, ADSS, ADSSLI, ADTRP, AEBP1, AEBP2, AEN, AES, AF130351.1, AF241726.2, AFAP1, AFAPILI, AFAPIL2, AFDN, AFFI, AFF2, AFF3, AFF4, AFGIL, AFG3L2, AFM, AFMID, AFP, AFTPH, AGA, AGAPI, AGAP2, AGAP3, AGAP4, AGAP5, AGAP6, AGAP9, AGBLI, AGBL2, AGBL3, AGBL4, AGBL5, AGER, AGFG1, AGFG2, AGGF1, AGK, AGL, AGMAT, AGMO, AGO1, AGO2, AGO3, AGO4, AGPATI, AGPAT2, AGPAT3, AGPAT4, AGPAT5, AGPS, AGR2, AGR3, AGRN, AGRP, AGT, AGTPBPI, AGTRI, AGTR2, AGTRAP, AGXT, AGXT2, AHCTFI, AHCY, AHCYLI, AHCYL2, AHDCI, AHI1, AHNAK, AHNAK2, AHR, AHRR, AHSAI, AHSA2, AHSG, AHSP, AICDA, AIDA, AIFI, AIFIL, AIFMI, AIFM2, AIFM3, AIGI, AIM2, AIMPI, AIMP2, AIP, AIPLI, AIRE, AJAPI, AJUBA, AKI, AK2, AK3, AK4, AK5, AK6, AK7, AK8, AK9, AKAINI, AKAPI, AKAP10, AKAP11, AKAP12, AKAP13, AKAP14, AKAP17A, AKAP2, AKAP3, AKAP4, AKAP5, AKAP6, AKAP7, AKAP8, AKAP8L, AKAP9, AKIPI, AKIRINI, AKIRIN2, AKNA, AKNADI, AKRIAI, AKRIBI, AKRIB10, AKRIB15, AKRICI, AKRIC2, AKRIC3, AKRIC4, AKRID1, AKRIE2, AKR7A2, AKR7A3, AKRYL, AKTI, AKTISI, AKT2, AKT3, AKTIP, AL020996.2, AL021154.3, AL021546.1, AL021997.3, AL022238.4, AL022318.4, AL024498.2, AL031708.1, AL032819.3, AL033529.1, AL035425.2, AL035460.1, AL049634.2, AL049650.1, AL049697.1, AL049779.1, AL049839.2, AL049844.1, AL049844.3, AL080251.1, AL096814.1, AL096870.1, AL109810.2, AL109811.4, AL109827.1, AL109936.3, AL109936.4, AL110118.2, AL110118.4, AL117258.1, AL117339.5, AL117348.2, AL121581.1, AL121594.3, AL121722.1, AL121753.1, AL121758.1, AL121845.2, AL121845.3, AL132671.2, AL132780.3, AL133352.1, AL133414.1, AL133414.2, AL136295.1, AL136295.3, AL136295.4, AL136295.5, AL136373.1, AL136531.2, AL138694.1, AL138752.2, AL138826.1, AL139011.2, AL139260.3, AL139300.1, AL139353.1, AL157392.5, AL159163.1, AL160275.1, AL160276.1, AL160396.2, AL161669.4, AL161911.1, AL162231.1, AL162231.3, AL163195.3, AL163636.2, AL353572.3, AL353588.1, AL354761.2, AL354822.1, AL355102.2, AL355315.1, AL355860.1, AL355916.3, AL355987.1, AL355987.3, AL356585.9, AL357673.1, AL358075.4, AL359736.1, AL359736.3, AL359922.1, AL360181.3, AL360181.5, AL365205.1, AL365214.3, AL365232.1, AL365273.2, AL391650.1, AL449266.1, AL451007.3, AL512428.1, AL512506.3, AL512785.2, AL513165.2, AL513523.10, AL513523.9, AL583836.1, AL589666.1, AL590132.1, AL590560.1, AL591806.3, AL592183.1, AL592490.1, AL593848.2, AL603832.3, AL645922.1, AL645941.2, AL662828.1, AL662852.6, AL662899.1, AL662899.2, AL662899.3, AL669918.1, AL672043.1, AL672142.1, AL691442.1, AL713999.1, AL772284.2, AL807752.6, AL807752.7, AL844853.2, AL845331.2, AL845464.1, AL928654.4, AL929554.1, AL929561.7, ALAD, ALASI, ALAS2, ALB, ALCAM, ALDH16A1, ALDH18A1, ALDHIA1, ALDHIA2, ALDHIA3, ALDHIBI, ALDHILI, ALDHIL2, ALDH2, ALDH3A1, ALDH3A2, ALDH3B1, ALDH3B2, ALDH4A1, ALDH5A1, ALDH6A1, ALDH7A1, ALDH8A1, ALDH9A1, ALDOA, ALDOB, ALDOC, ALGI, ALG10, ALG10B, ALG11, ALG12, ALG13, ALG14, ALGIL, ALGIL2, ALG2, ALG3, ALG5, ALG6, ALG8, ALG9, ALK, ALKALI, ALKAL2, ALKBHI, ALKBH2, ALKBH3, ALKBH4, ALKBH5, ALKBH6, ALKBH7, ALKBH8, ALLC, ALMS1, ALOX12, ALOX12B, ALOX15, ALOX15B, ALOX5, ALOX5AP, ALOXE3, ALPI, ALPKI, ALPK2, ALPK3, ALPL, ALPP, ALPPL2, ALS2, ALS2CL, ALS2CR₁₂, ALX1, ALX3, ALX4, ALYREF, AMACR, AMBN, AMBP, AMBRA1, AMD1, AMDHD1, AMDHD2, AMELX, AMELY, AMERI, AMER2, AMER3, AMFR, AMH, AMHR2, AMIGO1, AMIGO2, AMIGO3, AMMECRI, AMMECRIL, AMN, AMNI, AMOT, AMOTLI, AMOTL2, AMPDI, AMPD2, AMPD3, AMPH, AMT, AMTN, AMYIA, AMY1B, AMYIC, AMY2A, AMY2B, AMZI, AMZ2, ANAPCI, ANAPC10, ANAPC11, ANAPC13, ANAPC15, ANAPC16, ANAPC2, ANAPC4, ANAPC5, ANAPC7, ANG, ANGELI, ANGEL2, ANGPTI, ANGPT2, ANGPT4, ANGPTL1, ANGPTL2, ANGPTL3, ANGPTL4, ANGPTL5, ANGPTL6, ANGPTL7, ANGPTL8, ANHX, ANKI, ANK2, ANK3, ANKAR, ANKDDIA, ANKDDIB, ANKEF1, ANKFN1, ANKFY1, ANKH, ANKHD1, ANKHD1-EIF4EBP3, ANKIBI, ANKKI, ANKLEI, ANKLE2, ANKMY1, ANKMY2, ANKRA2, ANKRDI, ANKRD10, ANKRD11, ANKRD12, ANKRD13A, ANKRD13B, ANKRD13C, ANKRD13D, ANKRD16, ANKRD17, ANKRD18A, ANKRD18B, ANKRD2, ANKRD20A1, ANKRD20A2, ANKRD20A3, ANKRD20A4, ANKRD20A8P, ANKRD22, ANKRD23, ANKRD24, ANKRD26, ANKRD27, ANKRD28, ANKRD29, ANKRD30A, ANKRD30B, ANKRD30BL, ANKRD31, ANKRD33, ANKRD33B, ANKRD34A, ANKRD34B, ANKRD34C, ANKRD35, ANKRD36, ANKRD36B, ANKRD36C, ANKRD37, ANKRD39, ANKRD40, ANKRD42, ANKRD44, ANKRD45, ANKRD46, ANKRD49, ANKRD50, ANKRD52, ANKRD53, ANKRD54, ANKRD55, ANKRD6, ANKRD60, ANKRD61, ANKRD62, ANKRD63, ANKRD65, ANKRD66, ANKRD7, ANKRD9, ANKSIA, ANKSIB, ANKS3, ANKS4B, ANKS6, ANKUB1, ANKZF1, ANLN, ANO1, ANO10, ANO2, ANO3, ANO4, ANO5, ANO6, ANO7, ANO8, ANO9, ANOSI, ANP32A, ANP32B, ANP32D, ANP32E, ANPEP, ANTXR1, ANTXR2, ANTXRL, ANXA1, ANXA10, ANXA11, ANXA13, ANXA2, ANXA2R, ANXA3, ANXA4, ANXA5, ANXA6, ANXA7, ANXA8, ANXA8L1, ANXA9, AOAH, AOCI, AOC2, AOC3, AOX1, AP000275.2, AP000295.1, AP000311.1, AP000322.1, AP000349.1, AP000350.12, AP000350.4, AP000351.3, AP000351.7, AP000721.1, AP000781.2, AP001160.5, AP001273.2, AP001458.2, AP001781.3, AP001931.1, AP002360.1, AP002373.1, AP002495.1, AP002512.3, AP002512.4, AP002748.4, AP002990.1, AP003071.5, AP003108.2, AP003419.2, AP004243.1, AP006285.3, APIAR, APIB1, APIG1, APIG2, APIMI, AP1M2, AP1S1, AP1S2, AP1S3, AP2A1, AP2A2, AP2B1, AP2M1, AP2S1, AP3B1, AP3B2, AP3D1, AP3MI, AP3M2, AP3S1, AP3S2, AP4B1, AP4E1, AP4M1, AP4S1, AP5B1, AP5M1, AP5S1, AP5Z₁, APAFI, APBA1, APBA2, APBA3, APBBI, APBBIIP, APBB2, APBB3, APC, APC2, APCDD1, APCDDIL, APCS, APEH, APELA, APEX1, APEX2, APHIA, APHIB, API5, APIP, APLF, APLN, APLNR, APLP1, APLP2, APMAP, APOA1, APOA2, APOA4, APOA5, APOB, APOBEC1, APOBEC2, APOBEC3A, APOBEC3B, APOBEC3C, APOBEC3D, APOBEC3F, APOBEC3G, APOBEC3H, APOBEC4, APOBR, APOCI, APOC2, APOC3, APOC4, APOC4-APOC2, APOD, APOE, APOF, APOH, APOLI, APOL2, APOL3, APOL4, APOL5, APOL6, APOLD1, APOM, APOO, APOOL, APOPT1, APP, APPBP2, APPLI, APPL2, APRT, APTX, AQP1, AQP10, AQP11, AQP12A, AQP12B, AQP2, AQP3, AQP4, AQP5, AQP6, AQP7, AQP8, AQP9, AQR, AR, ARAF, ARAPI, ARAP2, ARAP3, ARC, ARCNI, AREG, ARELI, ARFI, ARF3, ARF4, ARF5, ARF6, ARFGAP1, ARFGAP2, ARFGAP3, ARFGEF1, ARFGEF2, ARFGEF3, ARFIP1, ARFIP2, ARFRP1, ARGI, ARG2, ARGFX, ARGLUI, ARHGAPI, ARHGAP10, ARHGAP11A, ARHGAP11B, ARHGAP12, ARHGAP15, ARHGAP17, ARHGAP18, ARHGAP19, ARHGAP19-SLIT1, ARHGAP20, ARHGAP21, ARHGAP22, ARHGAP23, ARHGAP24, ARHGAP25, ARHGAP26, ARHGAP27, ARHGAP28, ARHGAP29, ARHGAP30, ARHGAP31, ARHGAP32, ARHGAP33, ARHGAP35, ARHGAP36, ARHGAP39, ARHGAP4, ARHGAP40, ARHGAP42, ARHGAP44, ARHGAP45, ARHGAP5, ARHGAP6, ARHGAP8, ARHGAP9, ARHGDIA, ARHGDIB, ARHGDIG, ARHGEF1, ARHGEF10, ARHGEFIOL, ARHGEF11, ARHGEF12, ARHGEF15, ARHGEF16, ARHGEF17, ARHGEF18, ARHGEF19, ARHGEF2, ARHGEF25, ARHGEF26, ARHGEF28, ARHGEF3, ARHGEF33, ARHGEF35, ARHGEF37, ARHGEF38, ARHGEF39, ARHGEF4, ARHGEF40, ARHGEF5, ARHGEF6, ARHGEF7, ARHGEF9, ARIDIA, ARIDIB, ARID2, ARID3A, ARID3B, ARID3C, ARID4A, ARID4B, ARID5A, ARID5B, ARIHI, ARIH2, ARIH2OS, ARLI, ARL10, ARL11, ARL13A, ARL13B, ARL14, ARL14EP, ARL14EPL, ARL15, ARL16, ARL17A, ARL17B, ARL2, ARL2BP, ARL2-SNX15, ARL3, ARL4A, ARL4C, ARL4D, ARLSA, ARL5B, ARL5C, ARL6, ARL6IP1, ARL6IP4, ARL6IP5, ARL6IP6, ARL8A, ARL8B, ARL9, ARMCI, ARMCI0, ARMC12, ARMC2, ARMC3, ARMC4, ARMC5, ARMC6, ARMC7, ARMC8, ARMC9, ARMCX1, ARMCX2, ARMCX3, ARMCX4, ARMCX5, ARMCX6, ARMS2, ARMTI, ARNT, ARNT2, ARNTL, ARNTL2, ARPCIA, ARPCIB, ARPC2, ARPC3, ARPC4, ARPC4-TTLL3, ARPC5, ARPC5L, ARPIN, ARPP19, ARPP21, ARR3, ARRB1, ARRB2, ARRDC1, ARRDC2, ARRDC3, ARRDC4, ARRDC5, ARSA, ARSB, ARSD, ARSE, ARSF, ARSG, ARSH, ARSI, ARSJ, ARSK, ARTI, AR^{T3}, AR^{T4}, AR^{T5}, ARTN, ARVI, ARVCF, ARX, AS3MT, ASAHI, ASAH2, ASAH2B, ASAP1, ASAP2, ASAP3, ASB1, ASB10, ASB11, ASB12, ASB13, ASB14, ASB15, ASB16, ASB17, ASB18, ASB2, ASB3, ASB4, ASB5, ASB6, ASB7, ASB8, ASB9, ASCC1, ASCC2, ASCC3, ASCLI, ASCL2, ASCL3, ASCL4, ASCL5, ASFIA, ASFIB, ASGR1, ASGR2, ASHIL, ASH2L, ASIC1, ASIC2, ASIC3, ASIC4, ASIC5, ASIP, ASL, ASMT, ASMTL, ASNA1, ASNS, ASNSD1, ASPA, ASPDH, ASPG, ASPH, ASPHD1, ASPHD2, ASPM, ASPN, ASPRVI, ASPSCRI, ASRGLI, ASS1, ASTE1, ASTL, ASTNI, ASTN2, ASXL1, ASXL2, ASXL3, ASZI, ATADI, ATAD2, ATAD2B, ATAD3A, ATAD3B, ATAD3C, ATAD5, ATATI, ATCAY, ATEI, ATF1, ATF2, ATF3, ATF4, ATF5, ATF6, ATF6B, ATF7, ATF7IP, ATF7IP2, ATG10, ATG101, ATG12, ATG13, ATG14, ATG16L1, ATG16L2, ATG2A, ATG2B, ATG3, ATG4A, ATG4B, ATG4C, ATG4D, ATG5, ATG7, ATG9A, ATG9B, ATIC, ATLI, ATL2, ATL3, ATM, ATMIN, ATNI, ATOHI, ATOH7, ATOH8, ATOX1, ATP10A, ATP10B, ATP1OD, ATPIIA, ATP11B, ATP11C, ATP12A, ATP13A1, ATP13A2, ATP13A3, ATP13A4, ATP13A5, ATPIA1, ATPIA2, ATPIA3, ATPIA4, ATPIB1, ATPIB2, ATPIB3, ATP1B4, ATP23, ATP2A1, ATP2A2, ATP2A3, ATP2B1, ATP2B2, ATP2B3, ATP2B4, ATP2C₁, ATP2C₂, ATP4A, ATP4B, ATP5A1, ATP5B, ATP5C₁, ATP5D, ATP5E, ATP5EP2, ATP5F1, ATP5G1, ATP5G2, ATP5G3, ATP5H, ATP5I, ATP5J, ATP5J2, ATP5J2-PTCDI, ATP5L, ATP5L2, ATP50, ATP5S, ATP6API, ATP6APIL, ATP6AP2, ATP6VOAI, ATP6VOA2, ATP6VOA4, ATP6VOB, ATP6VOC, ATP6VODI, ATP6VOD2, ATP6VOE1, ATP6VOE2, ATP6VIA, ATP6VIB1, ATP6VIB2, ATP6VIC1, ATP6VIC2, ATP6VID, ATP6VIE1, ATP6VIE2, ATP6VIF, ATP6VIGI, ATP6VIG2, ATP6V1G2-DDX39B, ATP6VIG3, ATP6VIH, ATP7A, ATP7B, ATP8A1, ATP8A2, ATP8B1, ATP8B2, ATP8B3, ATP8B4, ATP9A, ATP9B, ATPAF1, ATPAF2, ATPIFI, ATR, ATRAID, ATRIP, ATRN, ATRNL1, ATRX, ATXNI, ATXN10, ATXNIL, ATXN2, ATXN2L, ATXN3, ATXN3L, ATXN7, ATXN7L1, ATXN7L2, ATXN7L3, ATXN7L3B, AUH, AUNIP, AUPI, AURKA, AURKAIP1, AURKB, AURKC, AUTS2, AVEN, AVIL, AVL9, AVP, AVPII, AVPRIA, AVPRIB, AVPR2, AWATI, AWAT2, AXDND1, AXINI, AXIN2, AXL, AZGP1, AZI2, AZINI, AZIN2, AZU1, B2M, B3GALNTI, B3GALNT2, B3GALT1, B3GALT2, B3GALT4, B3GALT5, B3GALT6, B3GAT1, B3GAT2, B3GAT3, B3GLCT, B3GNT2, B3GNT3, B3GNT4, B3GNT5, B3GNT6, B3GNT7, B3GNT8, B3GNT9, B3GNTL1, B4GALNT1, B4GALNT2, B4GALNT3, B4GALNT4, B4GALT1, B4GALT2, B4GALT3, B4GALT4, B4GALT5, B4GALT6, B4GALT7, B4GATI, B9D1, B9D2, BAALC, BAAT, BABAMI, BABAM2, BACE1, BACE2, BACHI, BACH2, BAD, BAGI, BAG2, BAG3, BAG4, BAG5, BAG6, BAGE3, BAHCCI, BAHDI, BAIAP2, BAIAP2L1, BAIAP2L2, BAIAP3, BAKI, BAMBI, BANFI, BANF2, BANK1, BANP, BAP1, BARD1, BARHLI, BARHL2, BARX1, BARX2, BASPI, BATF, BATF2, BATF3, BAX, BAZIA, BAZIB, BAZ2A, BAZ2B, BBC3, BBIP1, BBOF1, BBOX1, BBS1, BBS10, BBS12, BBS2, BBS4, BBS5, BBS7, BBS9, BBX, BCAM, BCAN, BCAP29, BCAP31, BCAR1, BCAR3, BCAS1, BCAS2, BCAS3, BCAS4, BCAT1, BCAT2, BCCIP, BCDIN3D, BCHE, BCKDHA, BCKDHB, BCKDK, BCL10, BCL11A, BCL11B, BCL2, BCL2A1, BCL2L1, BCL2L10, BCL2L11, BCL2L12, BCL2L13, BCL2L14, BCL2L15, BCL2L2, BCL2L2-PABPN1, BCL3, BCL6, BCL6B, BCL7A, BCL7B, BCL7C, BCL9, BCL9L, BCLAF1, BCLAF3, BCO1, BCO2, BCOR, BCORLI, BCR, BCSIL, BDH1, BDH2, BDKRB1, BDKRB2, BDNF, BDP1, BEAN1, BECNI, BECN2, BEGAIN, BEND2, BEND3, BEND4, BEND5, BEND6, BEND7, BESTI, BEST2, BEST3, BEST4, BET1, BETIL, BEXI, BEX2, BEX3, BEX4, BEX5, BFAR, BFSP1, BFSP2, BGLAP, BGN, BHLHA15, BHLHA9, BHLHB9, BHLHE22, BHLHE23, BHLHE40, BHLHE41, BHMG1, BHMT, BHMT2, BICCI, BICD1, BICD2, BICDL1, BICDL2, BICR^{A}, BICRAL, BID, BIK, BINI, BIN2, BIN3, BIRC2, BIRC3, BIRC5, BIRC6, BIRC7, BIRC8, BIVM, BIVM-ERCC5, BLACE, BLCAP, BLID, BLK, BLM, BLMH, BLNK, BLOCIS1, BLOCIS2, BLOCIS3, BLOCIS4, BLOCIS5, BLOCIS5-TXNDC5, BLOCIS6, BLVR^{A}, BLVRB, BLZF1, BMF, BMI1, BMP1, BMP10, BMP15, BMP2, BMP2K, BMP3, BMP4, BMP5, BMP6, BMP7, BMP8A, BMP8B, BMPER, BMPRIA, BMPRIB, BMPR2, BMS1, BMT2, BMX, BNC1, BNC2, BNIP1, BNIP2, BNIP3, BNIP3L, BNIPL, BOC, BOD1, BODIL1, BODIL2, BOK, BOLA1, BOLA2, BOLA2B, BOLA2-SMG1P6, BOLA3, BOLL, BOP1, BOR^{A}, BORCS5, BORCS6, BORCS7, BORCS7-ASMT, BORCS8, BORCS8-MEF2B, BPGM, BPHL, BPI, BPIFA1, BPIFA2, BPIFA3, BPIFB1, BPIFB2, BPIFB3, BPIFB4, BPIFB6, BPIFC, BPNTI, BPTF, BPY2, BPY2B, BPY2C, BRAF, BRAP, BRATI, BRCA1, BRCA2, BRCC3, BRD1, BRD2, BRD3, BRD4, BRD7, BRD8, BRD9, BRDT, BRF1, BRF2, BRI3, BRI3BP, BRICD5, BRINP1, BRINP2, BRINP3, BRIP1, BRIX1, BRKI, BRMS1, BRMSIL, BROX, BRPF1, BRPF3, BRS3, BRSK1, BRSK2, BRWD1, BRWD3, BSCL2, BSDC1, BSG, BSN, BSND, BSPHI, BSPRY, BST1, BST2, BSX, BTAF1, BTBD1, BTBD10, BTBD11, BTBD16, BTBD17, BTBD18, BTBD19, BTBD2, BTBD3, BTBD6, BTBD7, BTBD8, BTBD9, BTC, BTD, BTF3, BTF3L4, BTG1, BTG2, BTG3, BTG4, BTK, BTLA, BTNIAI, BTN2A1, BTN2A2, BTN3A1, BTN3A2, BTN3A3, BTNL2, BTNL3, BTNL8, BTNL9, BTRC, BUB1, BUBIB, BUBIB-PAK6, BUB3, BUD13, BUD23, BUD31, BVES, BX004987.1, BX072566.1, BX088645.1, BX248244.1, BX248413.4, BX248415.1, BX248516.1, BX276092.9, BYSL, BZW1, BZW2, C10orf10, C10orf105, C10orf107, C10orf113, C10orf120, C10orf126, C10orf128, C10orf142, C10orf35, C10orf53, C10orf55, C10orf62, C10orf67, C10orf71, C10orf76, C10orf82, C10orf88, C10orf90, C10orf95, C10orf99, C11orf1, C11orf16, C11orf21, C11orf24, C11orf40, C11orf42, C11orf45, Clorf49, C11orf52, C11orf53, C11orf54, C11orf57, C11orf58, C11orf63, C11orf65, C11orf68, C11orf70, C11orf71, C11orf74, C11orf80, C11orf84, C11orf86, C11orf87, C11orf88, C11orf91, C11orf94, C11orf95, C11orf96, C11orf97, C11orf98, C12orf10, C12orf29, C12orf4, C12orf40, C12orf42, C12orf43, C12orf45, C12orf49, C12orf50, C12orf54, C12orf56, C12orf57, C12orf60, C12orf65, C12orf66, C12orf71, C12orf73, C12orf74, C12orf75, C12orf76, C13orf42, C14orf105, C14orf119, C14orf132, C14orf159, C14orf166, C14orf177, C14orf178, C14orf180, C14orf2, C14orf28, C14orf37, C14orf39, C14orf79, C14orf80, C14orf93, C15orf38-AP3S2, C15orf39, C15orf40, C15orf41, C15orf48, C15orf52, C15orf53, C15orf59, C15orf61, C15orf62, C15orf65, C16orf45, C16orf46, C16orf52, C16orf54, C16orf58, C16orf59, C16orf62, C16orf70, C16orf71, C16orf72, C16orf74, C16orf78, C16orf82, C16orf86, C16orf87, C16orf89, C16orf90, C16orf91, C16orf92, C16orf95, C16orf96, C17orf100, C17orf105, C17orf107, C17orf113, C17orf47, C17orf49, C17orf50, C17orf51, C17orf53, C17orf58, C17orf62, C17orf64, C17orf67, C17orf74, C17orf75, C17orf78, C17orf80, C17orf97, C17orf98, C17orf99, C18orf21, C18orf25, C18orf32, C18orf54, C18orf63, C18orf8, C19orf12, C19orf18, C19orf24, C19orf25, C19orf33, C19orf35, C19orf38, C19orf44, C19orf47, C19orf48, C19orf53, C19orf54, C19orf57, C19orf60, C19orf66, C19orf67, C19orf68, C19orf70, C19orf71, C19orf73, C19orf81, C19orf84, CID, CIGALTI, CIGALTICI, CIGALTICIL, Clorf100, Clorf105, Clorf109, Clorf112, Clorf115, Clorf116, Clorf122, Clorf123, Clorf127, Clorf131, Clorf141, Clorf146, Clorf158, Clorf159, Clorf162, Clorf167, Clorf174, Clorf185, Clorf186, Clorf189, Clorf194, Clorf198, Clorf21, Clorf210, Clorf216, Clorf226, Clorf228, Clorf232, Clorf27, Clorf35, Clorf43, Clorf50, Clorf52, Clorf53, Clorf54, Clorf56, Clorf61, Clorf64, Clorf68, Clorf74, Clorf87, Clorf94, CIQA, CIQB, CIQBP, CIQC, CIQLI, CIQL2, CIQL3, CIQL4, CIQTNF1, CIQTNF12, C1QTNF2, C1QTNF3, C1QTNF3-AMACR, CIQTNF4, CIQTNF5, CIQTNF6, CIQTNF7, CIQTNF8, CIQTNF9, CIQTNF9B, CIR, CIRL, CIS, C2, C20orf141, C20orf144, C20orf173, C20orf194, C20orf196, C20orf202, C20orf204, C20orf24, C20orf27, C20orf85, C20orf96, C21orf140, C21orf2, C21orf33, C21orf58, C21orf59, C21orf62, C21orf91, C22orf15, C22orf23, C22orf31, C22orf39, C22orf42, C22orf46, C2CD2, C2CD2L, C2CD3, C2CD4A, C2CD4B, C2CD4C, C2CD4D, C2CD5, C2CD6, C2orf15, C2orf16, C2orf40, C2orf42, C2orf49, C2orf50, C2orf54, C2orf66, C2orf68, C2orf69, C2orf70, C2orf71, C2orf72, C2orf73, C2orf74, C2orf76, C2orf78, C2orf80, C2orf81, C2orf82, C2orf83, C2orf88, C2orf91, C3, C3ARI, C3orf14, C3orf18, C3orf20, C3orf22, C3orf30, C3orf33, C3orf35, C3orf36, C3orf38, C3orf49, C3orf52, C3orf56, C3orf58, C3orf62, C3orf67, C3orf70, C3orf80, C3orf84, C3orf85, C4A, C4B, C4B_2, C4BPA, C4BPB, C4orf17, C4orf19, C4orf22, C4orf26, C4orf3, C4orf32, C4orf33, C4orf36, C4orf45, C4orf46, C4orf47, C4orf48, C4orf50, C4orf51, C5, C5AR1, C5AR2, C5orf15, C5orf22, C5orf24, C5orf30, C5orf34, C5orf38, C5orf42, C5orf46, C5orf47, C5orf49, C5orf51, C5orf52, C5orf56, C5orf58, C5orf60, C5orf63, C5orf67, C6, C6orf10, C6orf106, C6orf118, C6orf120, C6orf132, C6orf136, C6orf141, C6orf15, C6orf163, C6orf201, C6orf203, C6orf222, C6orf223, C6orf226, C6orf229, C6orf47, C6orf48, C6orf52, C6orf58, C6orf62, C6orf89, C7, C7orf25, C7orf26, C7orf31, C7orf33, C7orf34, C7orf43, C7orf49, C7orf50, C7orf55-LUC7L2, C7orf57, C7orf61, C7orf72, C7orf73, C7orf77, C8A, C8B, C8G, C8orf22, C8orf33, C8orf34, C8orf37, C8orf4, C8orf44, C8orf44-SGK3, C8orf46, C8orf48, C8orf58, C8orf59, C8orf74, C8orf76, C8orf82, C8orf86, C8orf88, C8orf89, C9, C9orf116, C9orf129, C9orf131, C9orf135, C9orf152, C9orf153, C9orf16, C9orf172, C9orf24, C9orf3, C9orf40, C9orf43, C9orf47, C9orf50, C9orf57, C9orf64, C9orf66, C9orf72, C9orf78, C9orf84, C9orf85, C9orf92, CA1, CA10, CA11, CA12, CA13, CA14, CA2, CA3, CA4, CASA, CA5B, CA6, CA7, CA8, CA9, CAAP1, CAB39, CAB39L, CABIN1, CABLES1, CABLES2, CABP1, CABP2, CABP4, CABP5, CABP7, CABS1, CABYR, CACFD1, CACHD1, CACNAIA, CACNAIB, CACNAIC, CACNAID, CACNAIE, CACNAIF, CACNAIG, CACNAIH, CACNAII, CACNAIS, CACNA2D1, CACNA2D2, CACNA2D3, CACNA2D4, CACNB1, CACNB2, CACNB3, CACNB4, CACNG1, CACNG2, CACNG3, CACNG4, CACNG5, CACNG6, CACNG7, CACNG8, CACTIN, CACULI, CACYBP, CAD, CADMI, CADM2, CADM3, CADM4, CADPS, CADPS2, CAGE1, CALB1, CALB2, CALCA, CALCB, CALCOCO1, CALCOCO2, CALCR, CALCRL, CALDI, CALHMI, CALHM2, CALHM3, CALM1, CALM2, CALM3, CALML3, CALML4, CALML5, CALML6, CALNI, CALR, CALR3, CALU, CALY, CAMKI, CAMKID, CAMKIG, CAMK2A, CAMK2B, CAMK2D, CAMK2G, CAMK2N₁, CAMK2N₂, CAMK4, CAMKKI, CAMKK2, CAMKMT, CAMKV, CAMLG, CAMP, CAMSAPI, CAMSAP2, CAMSAP3, CAMTAI, CAMTA2, CANDI, CAND2, CANTI, CANX, CAPI, CAP2, CAPG, CAPNI, CAPN10, CAPN11, CAPN12, CAPN13, CAPN14, CAPN15, CAPN2, CAPN3, CAPN5, CAPN6, CAPN7, CAPN8, CAPN9, CAPNS1, CAPNS2, CAPRINI, CAPRIN2, CAPS, CAPS2, CAPSL, CAPZAI, CAPZA2, CAPZA3, CAPZB, CARD10, CARD11, CARD14, CARD16, CARD17, CARD18, CARD19, CARD6, CARD8, CARD9, CARF, CARHSP1, CARMI, CARMILI, CARMIL2, CARMIL3, CARNMTI, CARNSI, CARS, CARS2, CARTPT, CASCI, CASC10, CASC3, CASC4, CASDI, CASK, CASKINI, CASKIN2, CASP1, CASP10, CASP12, CASP14, CASP2, CASP3, CASP4, CASP5, CASP6, CASP7, CASP8, CASP8AP2, CASP9, CASQ1, CASQ2, CASR, CASS4, CAST, CASTORI, CASTOR2, CASZ1, CAT, CATIP, CATSPER1, CATSPER2, CATSPER3, CATSPER4, CATSPERB, CATSPERD, CATSPERE, CATSPERG, CATSPERZ, CAVI, CAV2, CAV3, CAVINI, CAVIN2, CAVIN3, CAVIN4, CBARP, CBFA2T2, CBFA2T3, CBFB, CBL, CBLB, CBLC, CBLL1, CBLN1, CBLN2, CBLN3, CBLN4, CBR1, CBR3, CBR4, CBS, CBSL, CBWDI, CBWD2, CBWD3, CBWD5, CBWD6, CBX1, CBX2, CBX3, CBX4, CBX5, CBX6, CBX7, CBX8, CBY1, CBY3, CC2DIA, CC2D1B, CC2D2A, CC2D2B, CCARI, CCAR2, CCBE1, CCDC102A, CCDC102B, CCDC103, CCDC105, CCDC106, CCDC107, CCDC110, CCDC112, CCDC113, CCDC114, CCDC115, CCDC116, CCDC117, CCDC12, CCDC120, CCDC121, CCDC122, CCDC124, CCDC125, CCDC126, CCDC127, CCDC129, CCDC13, CCDC130, CCDC134, CCDC136, CCDC137, CCDC138, CCDC14, CCDC140, CCDC141, CCDC142, CCDC144A, CCDC144NL, CCDC146, CCDC148, CCDC149, CCDC15, CCDC150, CCDC151, CCDC152, CCDC153, CCDC154, CCDC155, CCDC157, CCDC158, CCDC159, CCDC160, CCDC163, CCDC166, CCDC167, CCDC168, CCDC169, CCDC169-SOHLH2, CCDC17, CCDC170, CCDC171, CCDC172, CCDC173, CCDC174, CCDC175, CCDC177, CCDC178, CCDC179, CCDC18, CCDC180, CCDC181, CCDC182, CCDC183, CCDC184, CCDC185, CCDC186, CCDC187, CCDC188, CCDC189, CCDC190, CCDC191, CCDC192, CCDC194, CCDC195, CCDC196, CCDC197, CCDC22, CCDC24, CCDC25, CCDC27, CCDC28A, CCDC28B, CCDC3, CCDC30, CCDC32, CCDC33, CCDC34, CCDC36, CCDC38, CCDC39, CCDC40, CCDC42, CCDC43, CCDC47, CCDC50, CCDC51, CCDC54, CCDC57, CCDC58, CCDC59, CCDC6, CCDC60, CCDC61, CCDC62, CCDC63, CCDC65, CCDC66, CCDC68, CCDC69, CCDC7, CCDC70, CCDC71, CCDC71L, CCDC73, CCDC74A, CCDC74B, CCDC77, CCDC78, CCDC8, CCDC80, CCDC81, CCDC82, CCDC83, CCDC84, CCDC85A, CCDC85B, CCDC85C, CCDC86, CCDC87, CCDC88A, CCDC88B, CCDC88C, CCDC89, CCDC9, CCDC90B, CCDC91, CCDC92, CCDC93, CCDC94, CCDC96, CCDC97, CCERI, CCER2, CCHCR1, CCIN, CCK, CCKAR, CCKBR, CCL1, CCL11, CCL13, CCL14, CCL15, CCL15-CCL14, CCL16, CCL17, CCL18, CCL19, CCL2, CCL20, CCL21, CCL22, CCL23, CCL24, CCL25, CCL26, CCL27, CCL28, CCL3, CCL3L1, CCL3L3, CCL4, CCL4L2, CCL5, CCL7, CCL8, CCM2, CCM2L, CCNA1, CCNA2, CCNB1, CCNB1IP1, CCNB2, CCNB3, CCNC, CCNDI, CCND2, CCND3, CCNDBP1, CCNEI, CCNE2, CCNF, CCNG1, CCNG2, CCNH, CCNI, CCNI2, CCNJ, CCNJL, CCNK, CCNL1, CCNL2, CCNO, CCNT1, CCNT2, CCNY, CCNYL1, CCP110, CCPG1, CCR1, CCR10, CCR2, CCR3, CCR4, CCR5, CCR6, CCR7, CCR8, CCR9, CCRL2, CCS, CCSAP, CCSER1, CCSER2, CCT2, CCT3, CCT4, CCT5, CCT6A, CCT6B, CCT7, CCT8, CCT8L2, CCZ1, CCZ1B, CD101, CD109, CD14, CD151, CD160, CD163, CD163L1, CD164, CD164L2, CD177, CD180, CD19, CDIA, CD1B, CDIC, CDID, CDIE, CD2, CD200, CD200R¹, CD200RIL, CD207, CD209, CD22, CD226, CD24, CD244, CD247, CD248, CD27, CD274, CD276, CD28, CD2AP, CD2BP2, CD300A, CD300C, CD300E, CD300LB, CD300LD, CD300LF, CD300LG, CD302, CD320, CD33, CD34, CD36, CD37, CD38, CD3D, CD3E, CD3EAP, CD3G, CD4, CD40, CD40LG, CD44, CD46, CD47, CD48, CD5, CD52, CD53, CD55, CD58, CD59, CD5L, CD6, CD63, CD68, CD69, CD7, CD70, CD72, CD74, CD79A, CD79B, CD80, CD81, CD82, CD83, CD84, CD86, CD8A, CD8B, CD9, CD93, CD96, CD99, CD99L2, CDA, CDADCI, CDANI, CDC123, CDC14A, CDC14B, CDC16, CDC20, CDC20B, CDC23, CDC25A, CDC25B, CDC25C, CDC26, CDC27, CDC34, CDC37, CDC37L1, CDC40, CDC42, CDC42BPA, CDC42BPB, CDC42BPG, CDC42EP1, CDC42EP2, CDC42EP3, CDC42EP4, CDC42EP5, CDC42SE1, CDC42SE2, CDC45, CDC5L, CDC6, CDC7, CDC73, CDCA2, CDCA3, CDCA4, CDCA5, CDCA7, CDCA7L, CDCA8, CDCP1, CDCP2, CDHI, CDH10, CDH11, CDH12, CDH13, CDH15, CDH16, CDH17, CDH18, CDH19, CDH2, CDH₂O, CDH22, CDH23, CDH24, CDH26, CDH3, CDH4, CDH5, CDH6, CDH7, CDH8, CDH9, CDHRI, CDHR2, CDHR3, CDHR4, CDHR5, CDIPI, CDIPT, CDKI, CDK10, CDKIIA, CDK11B, CDK12, CDK13, CDK14, CDK15, CDK16, CDK17, CDK18, CDK19, CDK2, CDK20, CDK2AP1, CDK2AP2, CDK3, CDK4, CDK5, CDK5R¹, CDK5R², CDK5RAP1, CDK5RAP2, CDK5RAP3, CDK6, CDK7, CDK8, CDK9, CDKALI, CDKLI, CDKL2, CDKL3, CDKL4, CDKL5, CDKNIA, CDKNIB, CDKNIC, CDKN2A, CDKN2AIP, CDKN2AIPNL, CDKN2B, CDKN2C, CDKN2D, CDKN3, CDNF, CDO1, CDON, CDPF1, CDR1, CDR2, CDR2L, CDRTI, CDR^{T15}, CDR^{T15}L2, CDR^{T4}, CDS1, CDS2, CDSN, CDTI, CDV3, CDX1, CDX2, CDX4, CDY1, CDY1B, CDY2A, CDY2B, CDYL, CDYL2, CEACAMI, CEACAM16, CEACAM19, CEACAM20, CEACAM21, CEACAM3, CEACAM4, CEACAM5, CEACAM6, CEACAM7, CEACAM8, CEBPA, CEBPB, CEBPD, CEBPE, CEBPG, CEBPZ, CEBPZOS, CECR2, CEL, CELA1, CELA2A, CELA2B, CELA3A, CELA3B, CELF1, CELF2, CELF3, CELF4, CELF5, CELF6, CELSRI, CELSR2, CELSR3, CEMIP, CEMP1, CEND1, CENPA, CENPB, CENPBD1, CENPC, CENPE, CENPF, CENPH, CENPI, CENPJ, CENPK, CENPL, CENPM, CENPN, CENPO, CENPP, CENPQ, CENPS, CENPS-CORT, CENPT, CENPU, CENPV, CENPVL1, CENPVL2, CENPVL3, CENPW, CENPX, CEP104, CEP112, CEP120, CEP126, CEP128, CEP131, CEP135, CEP152, CEP162, CEP164, CEP170, CEP170B, CEP19, CEP192, CEP250, CEP290, CEP295, CEP295NL, CEP350, CEP41, CEP44, CEP55, CEP57, CEP57L1, CEP63, CEP68, CEP70, CEP72, CEP76, CEP78, CEP83, CEP85, CEP85L, CEP89, CEP95, CEP97, CEPTI, CERI, CERCAM, CERK, CERKL, CERSI, CERS2, CERS3, CERS4, CERS5, CERS6, CES1, CES2, CES3, CES4A, CES5A, CETNI, CETN2, CETN3, CETP, CFAP100, CFAP126, CFAP157, CFAP161, CFAP20, CFAP206, CFAP221, CFAP36, CFAP43, CFAP44, CFAP45, CFAP46, CFAP47, CFAP52, CFAP53, CFAP54, CFAP57, CFAP58, CFAP61, CFAP65, CFAP69, CFAP70, CFAP73, CFAP74, CFAP77, CFAP97, CFAP99, CFB, CFC1, CFCIB, CFD, CFDP1, CFH, CFHR1, CFHR2, CFHR3, CFHR4, CFHR5, CFI, CFL1, CFL2, CFLAR, CFP, CFTR, CGA, CGB1, CGB2, CGB3, CGB5, CGB7, CGB8, CGGBP1, CGN, CGNLI, CGREFI, CGRRF1, CH₂₅H, CHACI, CHAC2, CHAD, CHADL, CHAFIA, CHAFIB, CHAMPI, CHAT, CHCHDI, CHCHD10, CHCHD2, CHCHD3, CHCHD4, CHCHD5, CHCHD6, CHCHD7, CHD1, CHDIL, CHD2, CHD3, CHD4, CHD5, CHD6, CHD7, CHD8, CHD9, CHDH, CHEKI, CHEK2, CHERP, CHFR, CHGA, CHGB, CHI3L1, CHI3L2, CHIA, CHIC1, CHIC2, CHID1, CHITI, CHKA, CHKB, CHKB-CPTIB, CHL1, CHM, CHML, CHMPIA, CHMP1B, CHMP2A, CHMP2B, CHMP3, CHMP4A, CHMP4B, CHMP4C, CHMP5, CHMP6, CHMP7, CHNI, CHN2, CHODL, CHORDCI, CHP1, CHP2, CHPF, CHPF2, CHPTI, CHRACI, CHRD, CHRDLI, CHRDL2, CHRFAM7A, CHRM1, CHRM2, CHRM3, CHRM4, CHRM5, CHRNAI, CHRNA10, CHRNA2, CHRNA3, CHRNA4, CHRNA5, CHRNA6, CHRNA7, CHRNA9, CHRNB1, CHRNB2, CHRNB3, CHRNB4, CHRND, CHRNE, CHRNG, CHST1, CHST10, CHST11, CHST12, CHST13, CHST14, CHST15, CHST2, CHST3, CHST4, CHST5, CHST6, CHST7, CHST8, CHST9, CHSY1, CHSY3, CHTF18, CHTF8, CHTOP, CHUK, CHURCI, CHURCH-FNTB, CIAO1, CIAPINI, CIART, CIBI, CIB2, CIB3, CIB4, CIC, CIDEA, CIDEB, CIDEC, CIITA, CILP, CILP2, CINP, CIPC, CIRI, CIRBP, CISD1, CISD2, CISD3, CISH, CIT, CITED1, CITED2, CITED4, CIZI, CKAP2, CKAP2L, CKAP4, CKAP5, CKB, CKLF, CKLF-CMTMI, CKM, CKMTIA, CKMTIB, CKMT2, CKS1B, CKS2, CLASP1, CLASP2, CLASRP, CLC, CLCA1, CLCA2, CLCA4, CLCC1, CLCFI, CLCN1, CLCN2, CLCN3, CLCN4, CLCN5, CLCN6, CLCN7, CLCNKA, CLCNKB, CLDNI, CLDN10, CLDN11, CLDN12, CLDN14, CLDN15, CLDN16, CLDN17, CLDN18, CLDN19, CLDN2, CLDN20, CLDN22, CLDN23, CLDN24, CLDN25, CLDN3, CLDN34, CLDN4, CLDN5, CLDN6, CLDN7, CLDN8, CLDN9, CLDND1, CLDND2, CLEC10A, CLEC11A, CLEC12A, CLEC12B, CLEC14A, CLEC16A, CLEC17A, CLEC18A, CLEC18B, CLEC18C, CLEC19A, CLECIA, CLECIB, CLEC20A, CLEC2A, CLEC2B, CLEC2D, CLEC2L, CLEC3A, CLEC3B, CLEC4A, CLEC4C, CLEC4D, CLEC4E, CLEC4F, CLEC4G, CLEC4M, CLEC5A, CLEC6A, CLEC7A, CLEC9A, CLECLI, CLGN, CLHC1, CLIC1, CLIC2, CLIC3, CLIC4, CLIC5, CLIC6, CLINT1, CLIP1, CLIP2, CLIP3, CLIP4, CLK1, CLK2, CLK3, CLK4, CLLUI, CLLUIOS, CLMN, CLMP, CLN3, CLN5, CLN6, CLN8, CLNK, CLNSIA, CLOCK, CLP1, CLPB, CLPP, CLPS, CLPSLI, CLPSL2, CLPTM1, CLPTMIL, CLPX, CLRNI, CLRN2, CLRN3, CLSPN, CLSTN1, CLSTN2, CLSTN3, CLTA, CLTB, CLTC, CLTCL1, CLU, CLUAP1, CLUH, CLULI, CLVS1, CLVS2, CLYBL, CMA1, CMAS, CMBL, CMC1, CMC2, CMC4, CMIP, CMKLR1, CMPK1, CMPK2, CMSSI, CMTMI, CMTM2, CMTM3, CMTM4, CMTM5, CMTM6, CMTM7, CMTM8, CMTRI, CMTR2, CMYA5, CNBD1, CNBD2, CNBP, CNDP1, CNDP2, CNEPIRI, CNFN, CNGA1, CNGA2, CNGA3, CNGA4, CNGB1, CNGB3, CNIH1, CNIH2, CNIH3, CNIH4, CNKSR1, CNKSR2, CNKSR3, CNMD, CNN1, CNN2, CNN3, CNNMI, CNNM2, CNNM3, CNNM4, CNOT1, CNOT10, CNOT11, CNOT2, CNOT3, CNOT4, CNOT6, CNOT6L, CNOT7, CNOT8, CNOT9, CNP, CNPPDI, CNPY1, CNPY2, CNPY3, CNPY4, CNR1, CNR2, CNRIP1, CNST, CNTD1, CNTD2, CNTF, CNTFR, CNTLN, CNTNI, CNTN2, CNTN3, CNTN4, CNTN5, CNTN6, CNTNAP1, CNTNAP2, CNTNAP3, CNTNAP3B, CNTNAP4, CNTNAP5, CNTRL, CNTROB, COAI, COA3, COA4, COA5, COA6, COA7, COASY, COBL, COBLL1, COCH, COG1, COG2, COG3, COG4, COG5, COG6, COG7, COG8, COIL, COL10A1, COL11A1, COL11A2, COL12A1, COL13A1, COL14A1, COL15A1, COL16A1, COL17A1, COL18A1, COL19A1, COLIA1, COL1A2, COL20A1, COL21A1, COL22A1, COL23A1, COL24A1, COL25A1, COL26A1, COL27A1, COL28A1, COL2A1, COL3A1, COL4A1, COL4A2, COL4A3, COL4A3BP, COL4A4, COL4A5, COL4A6, COL5A1, COL5A2, COL5A3, COL6A1, COL6A2, COL6A3, COL6A5, COL6A6, COL7A1, COL8A1, COL8A2, COL9A1, COL9A2, COL9A3, COLCA2, COLEC10, COLEC11, COLEC12, COLGALTI, COLGALT2, COLQ, COMMDI, COMMD10, COMMD2, COMMD3, COMMD3-BMI1, COMMD4, COMMD5, COMMD6, COMMD7, COMMD8, COMMD9, COMP, COMT, COMTD1, COPA, COPB1, COPB2, COPE, COPG1, COPG2, COPRS, COPS2, COPS3, COPS4, COPS5, COPS6, COPS7A, COPS7B, COPS8, COPS9, COPZI, COPZ2, COQ10A, COQ10B, COQ2, COQ3, COQ4, COQ5, COQ6, COQ7, COQ8A, COQ8B, COQ9, CORIN, COROIA, CORO1B, CORO1C, CORO2A, CORO2B, CORO6, CORO7, CORO7-PAM16, CORT, COTL1, COX10, COX11, COX14, COX15, COX16, COX17, COX18, COX19, COX20, COX411, COX412, COX5A, COX5B, COX6A1, COX6A2, COX6B1, COX6B2, COX6C, COX7A1, COX7A2, COX7A2L, COX7B, COX7B2, COX7C, COX8A, COX8C, CP, CPA1, CPA2, CPA3, CPA4, CPA5, CPA6, CPAMD8, CPB1, CPB2, CPD, CPE, CPEB1, CPEB2, CPEB3, CPEB4, CPED1, CPLX1, CPLX2, CPLX3, CPLX4, CPM, CPN1, CPN2, CPNE1, CPNE2, CPNE3, CPNE4, CPNE5, CPNE6, CPNE7, CPNE8, CPNE9, CPO, CPOX, CPPED1, CPQ, CPS1, CPSF1, CPSF2, CPSF3, CPSF4, CPSF4L, CPSF6, CPSF7, CPTIA, CPT1B, CPTIC, CPT2, CPTP, CPVL, CPXCR1, CPXMI, CPXM2, CPZ, CRI, CRIL, CR2, CR354443.1, CR354443.2, CR388407.3, CR547123.3, CR753842.1, CR753845.2, CR759815.2, CR788250.1, CR847794.2, CR854858.1, CR933783.3, CR936239.1, CRABP1, CRABP2, CRACR2A, CRACR2B, CRADD, CRAMP1, CRAT, CRB1, CRB2, CRB3, CRBN, CRCP, CRCTI, CREB1, CREB3, CREB3L1, CREB3L2, CREB3L3, CREB3L4, CREB5, CREBBP, CREBL2, CREBRF, CREBZF, CREG1, CREG2, CRELD1, CRELD2, CREM, CRH, CRHBP, CRHR1, CRHR2, CRIM1, CRIP1, CRIP2, CRIP3, CRIPT, CRISP1, CRISP2, CRISP3, CRISPLD1, CRISPLD2, CRK, CRKL, CRLF1, CRLF2, CRLF3, CRLS1, CRMP1, CRNKLI, CRNN, CROCC, CROCC2, CROT, CRP, CRTACI, CRTAM, CRTAP, CRTC1, CRTC2, CRTC3, CRX, CRY1, CRY2, CRYAA, CRYAB, CRYBAI, CRYBA2, CRYBA4, CRYBBI, CRYBB2, CRYBB3, CRYBG1, CRYBG2, CRYBG3, CRYGA, CRYGB, CRYGC, CRYGD, CRYGN, CRYGS, CRYL1, CRYM, CRYZ, CRYZLI, CS, CSAD, CSAG1, CSAG2, CSAG3, CSDC2, CSDE1, CSEIL, CSF1, CSFIR, CSF2, CSF2R^{A}, CSF2RB, CSF3, CSF3R, CSGALNACTI, CSGALNACT2, CSH1, CSH2, CSHL1, CSK, CSMD1, CSMD2, CSMD3, CSNIS1, CSN2, CSN3, CSNKIA1, CSNKIAIL, CSNKID, CSNKIE, CSNKIG1, CSNKIG2, CSNKIG3, CSNK2A1, CSNK2A2, CSNK2A3, CSNK2B, CSPG4, CSPG5, CSPP1, CSRNP1, CSRNP2, CSRNP3, CSRP1, CSRP2, CSRP3, CSTI, CST11, CST2, CST3, CST4, CST5, CST6, CST7, CST8, CST9, CST9L, CSTA, CSTB, CSTF1, CSTF2, CSTF2T, CSTF3, CSTL1, CT45A1, CT45A10, CT45A2, CT45A3, CT45A5, CT45A6, CT45A7, CT45A8, CT45A9, CT476828.1, CT476828.10, CT476828.11, CT476828.12, CT476828.13, CT476828.14, CT476828.15, CT476828.16, CT476828.17, CT476828.18, CT476828.19, CT476828.2, CT476828.20, CT476828.21, CT476828.22, CT476828.3, CT476828.4, CT476828.5, CT476828.6, CT476828.7, CT476828.8, CT476828.9, CT47A1, CT47A10, CT47A11, CT47A12, CT47A2, CT47A3, CT47A4, CT47A5, CT47A6, CT47A7, CT47A8, CT47A9, CT47B1, CT55, CT62, CT83, CTAGIA, CTAGIB, CTAG2, CTAGE1, CTAGE15, CTAGE4, CTAGE5, CTAGE6, CTAGE8, CTAGE9, CTBP1, CTBP2, CTBS, CTC1, CTCF, CTCFL, CTDNEP1, CTDP1, CTDSP1, CTDSP2, CTDSPL, CTDSPL2, CTF1, CTGF, CTH, CTHRC1, CTIF, CTLA4, CTNNA1, CTNNA2, CTNNA3, CTNNALI, CTNNB1, CTNNBIP1, CTNNBLI, CTNND1, CTNND2, CTNS, CTPS1, CTPS2, CTR9, CTRB1, CTRB2, CTRC, CTRL, CTSA, CTSB, CTSC, CTSD, CTSE, CTSF, CTSG, CTSH, CTSK, CTSL, CTSO, CTSS, CTSV, CTSW, CTSZ, CTTN, CTTNBP2, CTTNBP2NL, CTUI, CTU2, CTXNI, CTXN2, CTXN3, CTXNDI, CU464060.1, CU633846.1, CU633980.1, CU633980.2, CU639417.1, CU639417.2, CUBN, CUEDC1, CUEDC2, CULI, CUL2, CUL3, CUL4A, CUL4B, CUL5, CUL7, CUL9, CUTA, CUTC, CUXI, CUX2, CUZDI, CWC15, CWC22, CWC25, CWC27, CWF19L1, CWF19L2, CWH43, CX3CL1, CX3CR1, CXADR, CXCLI, CXCL10, CXCL11, CXCL12, CXCL13, CXCL14, CXCL16, CXCL17, CXCL2, CXCL3, CXCL5, CXCL6, CXCL8, CXCL9, CXCR1, CXCR2, CXCR3, CXCR4, CXCR5, CXCR6, CXorf21, CXorf36, CXorf38, CXorf40A, CXorf40B, CXorf49, CXorf49B, CXorf51A, CXorf51B, CXorf56, CXorf57, CXorf58, CXorf65, CXorf66, CXorf67, CXXC1, CXXC4, CXXC5, CYB561, CYB561A3, CYB561D1, CYB561D2, CYB5A, CYB5B, CYB5D1, CYB5D2, CYB5R¹, CYB5R², CYB5R³, CYB5R⁴, CYB5RL, CYBA, CYBB, CYBRD1, CYC1, CYCS, CYFIP1, CYFIP2, CYGB, CYHR1, CYLC1, CYLC2, CYLD, CYP11A1, CYP11B1, CYP11B2, CYP17A1, CYP19A1, CYPIA1, CYPIA2, CYP1B1, CYP20A1, CYP21A2, CYP24A1, CYP26A1, CYP26B1, CYP26C1, CYP27A1, CYP27B1, CYP27C1, CYP2A13, CYP2A6, CYP2A7, CYP2B6, CYP2C18, CYP2C19, CYP2C8, CYP2C9, CYP2D6, CYP2D7, CYP2E1, CYP2F1, CYP2J2, CYP2R¹, CYP2S1, CYP2U1, CYP2W1, CYP39A1, CYP3A4, CYP3A43, CYP3A5, CYP3A7, CYP3A7-CYP3A51P, CYP46A1, CYP4A11, CYP4A22, CYP4B1, CYP4F11, CYP4F12, CYP4F2, CYP4F22, CYP4F3, CYP4F8, CYP4V2, CYP4X¹, CYP4ZI, CYP51A1, CYP7AI, CYP7B1, CYP8B1, CYR61, CYS1, CYSLTRI, CYSLTR2, CYSRTI, CYSTMI, CYTHI, CYTH2, CYTH3, CYTH4, CYTIP, CYTL1, CYYRI, D2HGDH, DAAMI, DAAM2, DABI, DAB2, DAB2IP, DACHI, DACH2, DACTI, DACT2, DACT3, DADI, DAGI, DAGLA, DAGLB, DALRD3, DAND5, DAO, DAOA, DAP, DAP3, DAPKI, DAPK2, DAPK3, DAPLI, DAPP1, DARS, DARS2, DAWI, DAXX, DAZ1, DAZ2, DAZ3, DAZ4, DAZAP1, DAZAP2, DAZL, DBF4, DBF4B, DBH, DBI, DBN1, DBNDD1, DBNDD2, DBNL, DBP, DBRI, DBT, DBX1, DBX2, DCAF1, DCAF10, DCAF11, DCAF12, DCAF12L1, DCAF12L2, DCAF13, DCAF15, DCAF16, DCAF17, DCAF4, DCAF4L1, DCAF4L2, DCAF5, DCAF6, DCAF7, DCAF8, DCAF8L1, DCAF8L2, DCAKD, DCANP1, DCBLD1, DCBLD2, DCC, DCD, DCDCl, DCDC2, DCDC2B, DCDC2C, DCHS1, DCHS2, DCK, DCLKI, DCLK2, DCLK3, DCLREIA, DCLREIB, DCLREIC, DCN, DCPIA, DCP1B, DCP2, DCPS, DCSTI, DCST2, DCSTAMP, DCT, DCTD, DCTNI, DCTN2, DCTN3, DCTN4, DCTN5, DCTN6, DCTPP1, DCUNID1, DCUNID2, DCUNID3, DCUNID4, DCUNID5, DCX, DCXR, DDA1, DDAHI, DDAH2, DDB1, DDB2, DDC, DDHD1, DDHD2, DDI1, DDI2, DDIAS, DDIT3, DDIT4, DDIT4L, DDN, DDO, DDOST, DDR1, DDR2, DDRGK1, DDT, DDTL, DDX1, DDX10, DDX11, DDX17, DDX18, DDX19A, DDX19B, DDX20, DDX21, DDX23, DDX24, DDX25, DDX27, DDX28, DDX31, DDX39A, DDX39B, DDX3X, DDX3Y, DDX4, DDX41, DDX42, DDX43, DDX46, DDX47, DDX49, DDX5, DDX50, DDX51, DDX52, DDX53, DDX54, DDX55, DDX56, DDX58, DDX59, DDX6, DDX60, DDX60L, DEAF1, DEC1, DECRI, DECR2, DEDD, DEDD2, DEF6, DEF8, DEFAI, DEFAIB, DEFA3, DEFA4, DEFA5, DEFA6, DEFBI, DEFB103A, DEFB103B, DEFB104A, DEFB104B, DEFB105A, DEFB105B, DEFB106A, DEFB106B, DEFB107A, DEFB107B, DEFB108B, DEFB110, DEFB112, DEFB113, DEFB114, DEFB115, DEFB116, DEFB118, DEFB119, DEFB121, DEFB123, DEFB124, DEFB125, DEFB126, DEFB127, DEFB128, DEFB129, DEFB130A, DEFB130B, DEFB131A, DEFB131B, DEFB132, DEFB133, DEFB134, DEFB135, DEFB136, DEFB4A, DEFB4B, DEGS1, DEGS2, DEK, DENNDIA, DENNDIB, DENNDIC, DENND2A, DENND2C, DENND2D, DENND3, DENND4A, DENND4B, DENND4C, DENND5A, DENND5B, DENND6A, DENND6B, DENR, DEPDC1, DEPDC1B, DEPDC4, DEPDC5, DEPDC7, DEPTOR, DER^{A}, DERL1, DERL2, DERL3, DES, DESII, DESI2, DET1, DEUP1, DEXI, DFFA, DFFB, DFNA5, DFNB59, DGATI, DGAT2, DGAT2L6, DGCR2, DGCR6, DGCR6L, DGCR8, DGKA, DGKB, DGKD, DGKE, DGKG, DGKH, DGKI, DGKK, DGKQ, DGKZ, DGUOK, DHCR24, DHCR7, DHDDS, DHDH, DHFR, DHFR2, DHH, DHODH, DHPS, DHRS1, DHRS11, DHRS12, DHRS13, DHRS2, DHRS3, DHRS4, DHRS4L2, DHRS7, DHRS7B, DHRS7C, DHRS9, DHRSX, DHTKD1, DHX15, DHX16, DHX29, DHX30, DHX32, DHX33, DHX34, DHX35, DHX36, DHX37, DHX38, DHX40, DHX57, DHX58, DHX8, DHX9, DIABLO, DIAPHI, DIAPH2, DIAPH3, DICERI, DIDO1, DIEXF, DIMTI, DIO1, DIO2, DIO3, DIP2A, DIP2B, DIP2C, DIRAS1, DIRAS2, DIRAS3, DIRCI, DIRC2, DIRC3, DIS3, DIS3L, DIS3L2, DISCI, DISP1, DISP2, DISP3, DIXDCI, DKCI, DKKI, DKK2, DKK3, DKK4, DKKL1, DLAT, DLC1, DLD, DLECI, DLEU7, DLG1, DLG2, DLG3, DLG4, DLG5, DLGAP1, DLGAP2, DLGAP3, DLGAP4, DLGAP5, DLK1, DLK2, DLL1, DLL3, DLL4, DLST, DLX1, DLX2, DLX3, DLX4, DLX5, DLX6, DMAC1, DMAC2, DMAP1, DMBTI, DMBX1, DMC1, DMD, DMGDH, DMKN, DMP1, DMPK, DMRTI, DMR^{T2}, DMR^{T3}, DMRTAI, DMRTA2, DMRTB1, DMRTC1, DMRTC1B, DMRTC2, DMTF1, DMTN, DMWD, DMXLI, DMXL2, DNA2, DNAAFI, DNAAF2, DNAAF3, DNAAF4, DNAAF5, DNAH1, DNAH10, DNAH10OS, DNAHII, DNAH12, DNAH14, DNAH17, DNAH2, DNAH3, DNAH5, DNAH6, DNAH7, DNAH8, DNAH9, DNAII, DNAI2, DNAJAI, DNAJA2, DNAJA3, DNAJA4, DNAJB1, DNAJB11, DNAJB12, DNAJB13, DNAJB14, DNAJB2, DNAJB4, DNAJB5, DNAJB6, DNAJB7, DNAJB8, DNAJB9, DNAJCI, DNAJC10, DNAJC11, DNAJC12, DNAJC13, DNAJC14, DNAJC15, DNAJC16, DNAJC17, DNAJC18, DNAJC19, DNAJC2, DNAJC21, DNAJC22, DNAJC24, DNAJC25, DNAJC25-GNG10, DNAJC27, DNAJC28, DNAJC3, DNAJC30, DNAJC4, DNAJC5, DNAJC5B, DNAJC5G, DNAJC6, DNAJC7, DNAJC8, DNAJC9, DNALI, DNAL4, DNALII, DNASEI, DNASEILI, DNASEIL2, DNASEIL3, DNASE2, DNASE2B, DND1, DNER, DNHD1, DNLZ, DNMI, DNMIL, DNM2, DNM3, DNMBP, DNMTI, DNMT3A, DNMT3B, DNMT3L, DNPEP, DNPHI, DNTT, DNTTIPI, DNTTIP2, DOC2A, DOC2B, DOCK1, DOCK10, DOCK11, DOCK2, DOCK3, DOCK4, DOCK5, DOCK6, DOCK7, DOCK8, DOCK9, DOHH, DOK1, DOK2, DOK3, DOK4, DOK5, DOK6, DOK7, DOLK, DOLPP1, DONSON, DOPEY1, DOPEY2, DOTIL, DPAGTI, DPCD, DPCR1, DPEP1, DPEP2, DPEP3, DPF1, DPF2, DPF3, DPH1, DPH2, DPH3, DPH5, DPH6, DPH7, DPM1, DPM2, DPM3, DPP10, DPP3, DPP4, DPP6, DPP7, DPP8, DPP9, DPPA2, DPPA3, DPPA4, DPPA5, DPRX, DPT, DPY19L1, DPY19L2, DPY19L3, DPY19L4, DPY30, DPYD, DPYS, DPYSL2, DPYSL3, DPYSL4, DPYSL5, DQX1, DRI, DRAMI, DRAM2, DRAP1, DRAXIN, DRC1, DRC3, DRC7, DRD1, DRD2, DRD3, DRD4, DRD5, DRG1, DRG2, DRGX, DRICHI, DROSHA, DRP2, DSC1, DSC2, DSC3, DSCAM, DSCAMLI, DSCC1, DSCR3, DSCR4, DSCR8, DSE, DSEL, DSG1, DSG2, DSG3, DSG4, DSN1, DSP, DSPP, DST, DSTN, DSTYK, DTD1, DTD2, DTHD1, DTL, DTNA, DTNB, DTNBP1, DTWD1, DTWD2, DTX1, DTX2, DTX3, DTX3L, DTX4, DTYMK, DUOX1, DUOX2, DUOXA1, DUOXA2, DUPDI, DUSIL, DUS2, DUS3L, DUS4L, DUSP1, DUSP10, DUSP11, DUSP12, DUSP13, DUSP14, DUSP15, DUSP16, DUSP18, DUSP19, DUSP2, DUSP21, DUSP22, DUSP23, DUSP26, DUSP27, DUSP28, DUSP3, DUSP4, DUSP5, DUSP6, DUSP7, DUSP8, DUSP9, DUT, DUX4, DUXA, DUXB, DVL1, DVL2, DVL3, DWORF, DXO, DYDC1, DYDC2, DYM, DYNAP, DYNCIHI, DYNCIII, DYNC1I2, DYNCILII, DYNCILI2, DYNC2H1, DYNC2LII, DYNLLI, DYNLL2, DYNLRBI, DYNLRB2, DYNLTI, DYNLT3, DYRKIA, DYRKIB, DYR^{k2}, DYR^{k3}, DYR^{k4}, DYSF, DYTN, DZANKI, DZIP1, DZIPIL, DZIP3, E2F1, E2F2, E2F3, E2F4, E2F5, E2F6, E2F7, E2F8, E4F1, EAF1, EAF2, EAPP, EARS2, EBAG9, EBF1, EBF2, EBF3, EBF4, EBI3, EBLN1, EBLN2, EBNAIBP2, EBP, EBPL, ECD, ECEI, ECE2, ECELI, ECHI, ECHDCI, ECHDC2, ECHDC3, ECHS1, ECI1, ECI2, ECM1, ECM2, ECSCR, ECSIT, ECT2, ECT2L, EDA, EDA2R, EDAR, EDARADD, EDC3, EDC4, EDDM13, EDDM3A, EDDM3B, EDEMI, EDEM2, EDEM3, EDF1, EDIL3, EDN1, EDN2, EDN3, EDNR^{A}, EDNRB, EDRF1, EEA1, EED, EEF1A1, EEF1A2, EEF1AKMTI, EEFIAKMT2, EEF1AKMT3, EEF1B2, EEFID, EEF1E1, EEF1E1-BLOC1S5, EEFIG, EEF2, EEF2K, EEF2KMT, EEFSEC, EEPD1, EFCABI, EFCAB10, EFCAB11, EFCAB12, EFCAB13, EFCAB14, EFCAB2, EFCAB3, EFCAB5, EFCAB6, EFCAB7, EFCAB8, EFCAB9, EFCCI, EFEMP1, EFEMP2, EFHB, EFHC1, EFHC2, EFHD1, EFHD2, EFL1, EFNA1, EFNA2, EFNA3, EFNA4, EFNA5, EFNB1, EFNB2, EFNB3, EFR3A, EFR3B, EFS, EFTUD2, EGF, EGFL6, EGFL7, EGFL8, EGFLAM, EGFR, EGLN1, EGLN2, EGLN3, EGR1, EGR2, EGR3, EGR4, EHBP1, EHBPIL1, EHD1, EHD2, EHD3, EHD4, EHF, EHHADH, EHMT1, EHMT2, EI24, EID1, EID2, EID2B, EID3, EIF1, EIFIAD, EIFIAX, EIFIAY, EIFIB, EIF2A, EIF2AK1, EIF2AK2, EIF2AK3, EIF2AK4, EIF2B1, EIF2B2, EIF2B3, EIF2B4, EIF2B5, EIF2D, EIF2S1, EIF2S2, EIF2S3, EIF3A, EIF3B, EIF3C, EIF3CL, EIF3D, EIF3E, EIF3F, EIF3G, EIF3H, EIF3I, EIF3J, EIF3K, EIF3L, EIF3M, EIF4A1, EIF4A2, EIF4A3, EIF4B, EIF4E, EIF4E1B, EIF4E2, EIF4E3, EIF4EBP1, EIF4EBP2, EIF4EBP3, EIF4ENIF1, EIF4G1, EIF4G2, EIF4G3, EIF4H, EIF5, EIF5A, EIF5A2, EIF5AL1, EIF5B, EIF6, EIPRI, ELACI, ELAC2, ELANE, ELAVLI, ELAVL2, ELAVL3, ELAVL4, ELF1, ELF2, ELF3, ELF4, ELF5, ELFN1, ELFN2, ELK1, ELK3, ELK4, ELL, ELL2, ELL3, ELMO1, ELMO2, ELMO3, ELMOD1, ELMOD2, ELMOD3, ELMSANI, ELN, ELOA, ELOA2, ELOA3, ELOA3B, ELOA3C, ELOA3D, ELOB, ELOC, ELOFI, ELOVLI, ELOVL2, ELOVL3, ELOVL4, ELOVL5, ELOVL6, ELOVL7, ELP1, ELP2, ELP3, ELP4, ELP5, ELP6, ELSPBPI, EMB, EMC1, EMC10, EMC2, EMC3, EMC4, EMC6, EMC7, EMC8, EMC9, EMCN, EMD, EME1, EME2, EMG1, EMID1, EMILIN1, EMILIN2, EMILIN3, EML1, EML2, EML3, EML4, EML5, EML6, EMP1, EMP2, EMP3, EMSY, EMX1, EMX2, EN1, EN2, ENAH, ENAM, ENCI, ENDOD1, ENDOG, ENDOU, ENDOV, ENG, ENGASE, ENHO, ENKD1, ENKUR, ENO1, ENO2, ENO3, EN04, ENOPHI, ENOSF1, ENOX1, ENOX2, ENPEP, ENPPI, ENPP2, ENPP3, ENPP4, ENPP5, ENPP6, ENPP7, ENSA, ENTHD1, ENTPD1, ENTPD2, ENTPD3, ENTPD4, ENTPD5, ENTPD6, ENTPD7, ENTPD8, ENY2, EOGT, EOMES, EP300, EP400, EPAS1, EPB41, EPB41L1, EPB41L2, EPB41L3, EPB41L4A, EPB41L4B, EPB41L5, EPB42, EPC1, EPC2, EPCAM, EPDR1, EPG5, EPGN, EPHA1, EPHA10, EPHA2, EPHA3, EPHA4, EPHA5, EPHA6, EPHA7, EPHA8, EPHB1, EPHB2, EPHB3, EPHB4, EPHB6, EPHX1, EPHX2, EPHX3, EPHX4, EPM2A, EPM2AIP1, EPNI, EPN2, EPN3, EPO, EPOP, EPOR, EPPIN, EPPIN-WFDC6, EPPK1, EPRS, EPS15, EPS15L1, EPS8, EPS8L1, EPS8L2, EPS8L3, EPSTII, EPX, EPYC, EQTN, ERALI, ERAPI, ERAP2, ERAS, ERBB2, ERBB3, ERBB4, ERBIN, ERCI, ERC2, ERCC1, ERCC2, ERCC3, ERCC4, ERCC5, ERCC6, ERCC6L, ERCC6L2, ERCC8, EREG, ERF, ERFE, ERG, ERG28, ERGICI, ERGIC2, ERGIC3, ERH, ERII, ERI2, ERI3, ERICHI, ERICH2, ERICH3, ERICH4, ERICH5, ERICH6, ERICH6B, ERLECI, ERLINI, ERLIN2, ERMAP, ERMARD, ERMN, ERMP1, ERN1, ERN2, EROIA, ERO1B, ERP27, ERP29, ERP44, ERRFI1, ERV3-1, ERVFRD-1, ERVMER34-1, ERVV-1, ERVV-2, ERVW-1, ESAM, ESCO1, ESCO2, ESD, ESF1, ESMI, ESPLI, ESPN, ESPNL, ESR1, ESR2, ESRP1, ESRP2, ESRR^{A}, ESRRB, ESRRG, ESS2, ESX1, ESYT1, ESYT2, ESYT3, ETAA1, ETDA, ETDB, ETDC, ETF1, ETFA, ETFB, ETFBKMT, ETFDH, ETFRF1, ETHEI, ETNK1, ETNK2, ETNPPL, ETS1, ETS2, ETVI, ETV2, ETV3, ETV3L, ETV4, ETV5, ETV6, ETV7, EVAIA, EVAIB, EVAIC, EVC, EVC2, EVI2A, EVI2B, EVI5, EVIL, EVL, EVPL, EVPLL, EVX1, EVX2, EWSRI, EXD1, EXD2, EXD3, EXO1, EXO5, EXOCI, EXOCIL, EXOC2, EXOC3, EXOC3L1, EXOC3L2, EXOC3L4, EXOC4, EXOC5, EXOC6, EXOC6B, EXOC7, EXOC8, EXOG, EXOSCI, EXOSC10, EXOSC2, EXOSC3, EXOSC4, EXOSC5, EXOSC6, EXOSC7, EXOSC8, EXOSC9, EXPH5, EXT1, EXT2, EXTL1, EXTL2, EXTL3, EYA1, EYA2, EYA3, EYA4, EYS, EZH1, EZH2, EZR, F10, F11, F1IR, F12, F13A1, F13B, F2, F2R, F2RL1, F2RL2, F2RL3, F3, F5, F7, F8, F8A1, F8A2, F8A3, F9, FA2H, FAAH, FAAH2, FAAP100, FAAP20, FAAP24, FABPI, FABP12, FABP2, FABP3, FABP4, FABP5, FABP6, FABP7, FABP9, FADD, FADS1, FADS2, FADS3, FADS6, FAFI, FAF2, FAH, FAHD1, FAHD2A, FAHD2B, FAIM, FAIM2, FAM102A, FAM102B, FAM103A1, FAM104A, FAM104B, FAM105A, FAM106A, FAM107A, FAM107B, FAM109A, FAM109B, FAM110A, FAM110B, FAM110C, FAM110D, FAM111A, FAM111B, FAM114A1, FAM114A2, FAM117A, FAM117B, FAM118A, FAM118B, FAM120A, FAM120AOS, FAM120B, FAM120C, FAM122A, FAM122B, FAM122C, FAM124A, FAM124B, FAM126A, FAM126B, FAM129A, FAM129B, FAM129C, FAM131A, FAM131B, FAM131C, FAM133A, FAM133B, FAM135A, FAM135B, FAM136A, FAM13A, FAM13B, FAM13C, FAM149A, FAM149B1, FAM151A, FAM151B, FAM153A, FAM153B, FAM153C, FAM155A, FAM155B, FAM156A, FAM156B, FAM159A, FAM159B, FAM160A1, FAM160A2, FAM160B1, FAM160B2, FAM161A, FAM161B, FAM162A, FAM162B, FAM163A, FAM163B, FAM166A, FAM166B, FAM167A, FAM167B, FAM168A, FAM168B, FAM169A, FAM169B, FAM170A, FAM170B, FAM171A1, FAM171A2, FAM171B, FAM172A, FAM173A, FAM173B, FAM174A, FAM174B, FAM177A1, FAM177B, FAM178B, FAM180A, FAM180B, FAM181A, FAM181B, FAM182B, FAM183A, FAM184A, FAM184B, FAM185A, FAM186A, FAM186B, FAM187A, FAM187B, FAM189A1, FAM189A2, FAM189B, FAM192A, FAM193A, FAM193B, FAM196A, FAM196B, FAM198A, FAM198B, FAM199X, FAM19A1, FAM19A2, FAM19A3, FAM19A4, FAM19A5, FAM200A, FAM200B, FAM204A, FAM205A, FAM205C, FAM206A, FAM207A, FAM208A, FAM208B, FAM209A, FAM209B, FAM20A, FAM20B, FAM20C, FAM210A, FAM210B, FAM212A, FAM212B, FAM213A, FAM213B, FAM214A, FAM214B, FAM216A, FAM216B, FAM217A, FAM217B, FAM218A, FAM219A, FAM219B, FAM220A, FAM221A, FAM221B, FAM222A, FAM222B, FAM227A, FAM227B, FAM228A, FAM228B, FAM229A, FAM229B, FAM230A, FAM231A, FAM231B, FAM231C, FAM231D, FAM234A, FAM234B, FAM236A, FAM236B, FAM236C, FAM236D, FAM237A, FAM237B, FAM240A, FAM240B, FAM24A, FAM24B, FAM25A, FAM25C, FAM25G, FAM26D, FAM26E, FAM26F, FAM32A, FAM35A, FAM3A, FAM3B, FAM3C, FAM3D, FAM43A, FAM43B, FAM45A, FAM46A, FAM46B, FAM46C, FAM46D, FAM47A, FAM47B, FAM47C, FAM47E, FAM47E-STBD1, FAM49A, FAM49B, FAM50A, FAM50B, FAM53A, FAM53B, FAM53C, FAM57A, FAM57B, FAM58A, FAM60A, FAM69A, FAM69B, FAM69C, FAM71A, FAM71B, FAM7IC, FAM7ID, FAM71E1, FAM71E2, FAM7IF1, FAM7IF2, FAM72A, FAM72B, FAM72C, FAM72D, FAM76A, FAM76B, FAM78A, FAM78B, FAM81A, FAM81B, FAM83A, FAM83B, FAM83C, FAM83D, FAM83E, FAM83F, FAM83G, FAM83H, FAM84A, FAM84B, FAM86B1, FAM86B2, FAM86C1, FAM89A, FAM89B, FAM8A1, FAM90A1, FAM90A26, FAM91A1, FAM92A, FAM92B, FAM95C, FAM96A, FAM96B, FAM98A, FAM98B, FAM98C, FAM9A, FAM9B, FAM9C, FANI, FANCA, FANCB, FANCC, FANCD2, FANCD2OS, FANCE, FANCF, FANCG, FANCI, FANCL, FANCM, FANKI, FAP, FARI, FAR2, FARPI, FARP2, FARS2, FARSA, FARSB, FAS, FASLG, FASN, FASTK, FASTKD1, FASTKD2, FASTKD3, FASTKD5, FATI, FAT2, FAT3, FAT4, FATEI, FAU, FAXC, FAXDC2, FBF1, FBL, FBLIMI, FBLLI, FBLNI, FBLN2, FBLN5, FBLN7, FBN1, FBN2, FBN3, FBP1, FBP2, FBRS, FBRSLI, FBXL12, FBXL13, FBXL14, FBXL15, FBXL16, FBXL17, FBXL18, FBXL19, FBXL2, FBXL20, FBXL22, FBXL3, FBXL4, FBXL5, FBXL6, FBXL7, FBXL8, FBXO10, FBXO11, FBXO15, FBXO16, FBXO17, FBXO18, FBX02, FBXO21, FBXO22, FBXO24, FBXO25, FBXO27, FBXO28, FBXO3, FBXO30, FBXO31, FBXO32, FBXO33, FBXO34, FBXO36, FBXO38, FBXO39, FBXO4, FBXO40, FBXO41, FBXO42, FBXO43, FBXO44, FBXO45, FBX046, FBX047, FBXO48, FBX05, FBX06, FBX07, FBX08, FBX09, FBXW10, FBXW11, FBXW12, FBXW2, FBXW4, FBXW5, FBXW7, FBXW8, FBXW9, FCAMR, FCAR, FCERIA, FCERIG, FCER2, FCF1, FCGBP, FCGRIA, FCGRIB, FCGR2A, FCGR2B, FCGR2C, FCGR3A, FCGR3B, FCGRT, FCHO1, FCHO2, FCHSD1, FCHSD2, FCMR, FCN1, FCN2, FCN3, FCRL1, FCRL2, FCRL3, FCRL4, FCRL5, FCRL6, FCRLA, FCRLB, FDCSP, FDFTI, FDPS, FDX1, FDX2, FDXACB1, FDXR, FECH, FEMIA, FEMIB, FEMIC, FEN1, FER, FER1L5, FER1L6, FERD3L, FERMTI, FERMT2, FERMT3, FES, FETUB, FEV, FEZ1, FEZ2, FEZF1, FEZF2, FFARI, FFAR2, FFAR3, FFAR4, FGA, FGB, FGD1, FGD2, FGD3, FGD4, FGD5, FGD6, FGF1, FGF10, FGF11, FGF12, FGF13, FGF14, FGF16, FGF17, FGF18, FGF19, FGF2, FGF20, FGF21, FGF22, FGF23, FGF3, FGF4, FGF5, FGF6, FGF7, FGF8, FGF9, FGFBP1, FGFBP2, FGFBP3, FGFR1, FGFRIOP, FGFR1OP2, FGFR2, FGFR3, FGFR4, FGFRL1, FGG, FGGY, FGL1, FGL2, FGR, FH, FHAD1, FHDC1, FHIT, FHL1, FHL2, FHL3, FHL5, FHOD1, FHOD3, FIBCD1, FIBIN, FIBP, FICD, FIG. 4, FIGLA, FIGN, FIGNL1, FIGNL2, FILIP1, FILIPIL, FIPIL1, FISI, FITM1, FITM2, FIZ1, FJX1, FKBP10, FKBP11, FKBP14, FKBP15, FKBPIA, FKBP1B, FKBPIC, FKBP2, FKBP3, FKBP4, FKBP5, FKBP6, FKBP7, FKBP8, FKBP9, FKBPL, FKRP, FKTN, FLADI, FLCN, FLG, FLG2, FLII, FLII, FLNA, FLNB, FLNC, FLOTI, FLOT2, FLR^{T1}, FLR^{T2}, FLR^{T3}, FLT1, FLT3, FLT3LG, FLT4, FLVCR1, FLVCR2, FLYWCH1, FLYWCH2, FMC1, FMN1, FMN2, FMNL1, FMNL2, FMNL3, FMO1, FM02, FMO3, FM04, FM05, FMOD, FMRI, FMRINB, FN1, FN3K, FN3KRP, FNBP1, FNBPIL, FNBP4, FNDC1, FNDC10, FNDC11, FNDC3A, FNDC3B, FNDC4, FNDC5, FNDC7, FNDC8, FNDC9, FNIP1, FNIP2, FNTA, FNTB, FO681492.1, FO681542.1, FOCAD, FOLHI, FOLR1, FOLR2, FOLR3, FOPNL, FOS, FOSB, FOSLI, FOSL2, FOXA1, FOXA2, FOXA3, FOXB1, FOXB2, FOXC1, FOXC2, FOXD1, FOXD2, FOXD3, FOXD4, FOXD4L1, FOXD4L3, FOXD4L4, FOXD4L5, FOXD4L6, FOXE1, FOXE3, FOXF1, FOXF2, FOXG1, FOXHI, FOXI1, FOXI2, FOXI3, FOXJI, FOXJ2, FOXJ3, FOXK1, FOXK2, FOXLI, FOXL2, FOXL2NB, FOXM1, FOXN1, FOXN2, FOXN3, FOXN4, FOXO1, FOX03, FOXO4, FOX06, FOXP1, FOXP2, FOXP3, FOXP4, FOXQ1, FOXR1, FOXR2, FOXRED1, FOXRED2, FOXSI, FP236240.1, FP565260.1, FP565260.2, FP565260.3, FP565260.4, FP565260.6, FP565260.7, FP565324.1, FP565324.2, FPGS, FPGT, FPGT-TNNI3K, FPR1, FPR2, FPR3, FRA10ACI, FRASI, FRATI, FRAT2, FREMI, FREM2, FREM3, FRG1, FRG2, FRG2B, FRG2C, FRK, FRMD1, FRMD3, FRMD4A, FRMD4B, FRMD5, FRMD6, FRMD7, FRMD8, FRMPD1, FRMPD2, FRMPD3, FRMPD4, FRRS1, FRRSIL, FRS2, FRS3, FRY, FRYL, FRZB, FSBP, FSCB, FSCN1, FSCN2, FSCN3, FSD1, FSDIL, FSD2, FSHB, FSHR, FSIP1, FSIP2, FST, FSTL1, FSTL3, FSTL4, FSTL5, FTCD, FTCDNL1, FTHI, FTHL17, FTL, FTMT, FTO, FTSJ1, FTSJ3, FUBP1, FUBP3, FUCA1, FUCA2, FUK, FUNDC1, FUNDC2, FUOM, FURIN, FUS, FUT1, FUT10, FUT11, FUT2, FUT3, FUT4, FUT5, FUT6, FUT7, FUT8, FUT9, FUZ, FXN, FXR1, FXR2, FXYD1, FXYD2, FXYD3, FXYD4, FXYD5, FXYD6, FXYD6-FXYD2, FXYD7, FYB1, FYB2, FYCO1, FYN, FYTTD1, FZD1, FZD10, FZD2, FZD3, FZD4, FZD5, FZD6, FZD7, FZD8, FZD9, FZR1, GOS2, G2E3, G3BP1, G3BP2, G6PC, G6PC2, G6PC3, G6PD, GAA, GABI, GAB2, GAB3, GAB4, GABARAP, GABARAPLI, GABARAPL2, GABBRI, GABBR2, GABPA, GABPB1, GABPB2, GABRA1, GABRA2, GABRA3, GABRA4, GABRA5, GABRA6, GABRBI, GABRB2, GABRB3, GABRD, GABRE, GABRGI, GABRG2, GABRG3, GABRP, GABRQ, GABRRI, GABRR2, GABRR3, GADI, GAD2, GADD45A, GADD45B, GADD45G, GADD45GIP1, GADLI, GAGE1, GAGE10, GAGE12B, GAGE12C, GAGE12D, GAGE12E, GAGE12F, GAGE12G, GAGE12H, GAGE12J, GAGE13, GAGE2A, GAGE2E, GAK, GAL, GAL3ST1, GAL3ST2, GAL3ST3, GAL3ST4, GALC, GALE, GALKI, GALK2, GALM, GALNS, GALNTI, GALNT10, GALNT11, GALNT12, GALNT13, GALNT14, GALNT15, GALNT16, GALNT17, GALNT18, GALNT2, GALNT3, GALNT4, GALNT5, GALNT6, GALNT7, GALNT8, GALNT9, GALNTL5, GALNTL6, GALP, GALRI, GALR2, GALR3, GALT, GAMT, GAN, GANAB, GANC, GAP43, GAPDH, GAPDHS, GAPT, GAPVDI, GARI, GAREMI, GAREM2, GARNL3, GARS, GART, GAS1, GAS2, GAS2L1, GAS2L2, GAS2L3, GAS6, GAS7, GAS8, GAST, GATAI, GATA2, GATA3, GATA4, GATA5, GATA6, GATADI, GATAD2A, GATAD2B, GATB, GATC, GATDI, GATM, GATS, GBA, GBA2, GBA3, GBEI, GBFI, GBGTI, GBPI, GBP2, GBP3, GBP4, GBP5, GBP6, GBP7, GBX1, GBX2, GC, GCA, GCAT, GCC1, GCC2, GCDH, GCFC2, GCG, GCGR, GCHI, GCHFR, GCK, GCKR, GCLC, GCLM, GCM1, GCM2, GCN1, GCNA, GCNTI, GCNT2, GCNT3, GCNT4, GCNT7, GCOMI, GCSAM, GCSAML, GCSH, GDA, GDAPI, GDAPILI, GDAP2, GDE1, GDF1, GDF10, GDF11, GDF15, GDF2, GDF3, GDF5, GDF5OS, GDF6, GDF7, GDF9, GDI1, GDI2, GDNF, GDPD1, GDPD2, GDPD3, GDPD4, GDPD5, GDPGP1, GEM, GEMIN2, GEMIN4, GEMIN5, GEMIN6, GEMIN7, GEMIN8, GENI, GET4, GFAP, GFER, GFII, GFIIB, GFMI, GFM2, GFOD1, GFOD2, GFPT1, GFPT2, GFRA1, GFRA2, GFRA3, GFRA4, GFRAL, GFY, GGA1, GGA2, GGA3, GGACT, GGCT, GGCX, GGH, GGN, GGNBP2, GGPS1, GGT1, GGT2, GGT5, GGT6, GGT7, GGTLC1, GGTLC2, GGTLC3, GHI, GH2, GHDC, GHITM, GHR, GHRH, GHRHR, GHRL, GHSR, GID4, GID8, GIF, GIGYFI, GIGYF2, GIMAPI, GIMAP1-GIMAP5, GIMAP2, GIMAP4, GIMAP5, GIMAP6, GIMAP7, GIMAP8, GIMDI, GINI, GINMI, GINS1, GINS2, GINS3, GINS4, GIP, GIPC1, GIPC2, GIPC3, GIPR, GITI, GIT2, GJAI, GJA10, GJA3, GJA4, GJA5, GJA8, GJA9, GJB1, GJB2, GJB3, GJB4, GJB5, GJB6, GJB7, GJC1, GJC2, GJC3, GJD2, GJD3, GJD4, GJE1, GK, GK2, GK3P, GK5, GKAP1, GKN1, GKN2, GLA, GLB1, GLBIL, GLB1L2, GLBIL3, GLCCII, GLCE, GLDC, GLDN, GLE1, GLG1, GLII, GLI2, GLI3, GLI4, GLIPRI, GLIPRILI, GLIPRIL2, GLIPR2, GLISI, GLIS2, GLIS3, GLMN, GLMP, GLO1, GLOD4, GLOD5, GLPIR, GLP2R, GLRAI, GLRA2, GLRA3, GLRA4, GLRB, GLRX, GLRX2, GLRX3, GLRX5, GLS, GLS2, GLTID1, GLT6D1, GLT8D1, GLT8D2, GLTP, GLTPD2, GLUDI, GLUD2, GLUL, GLYAT, GLYATLI, GLYATLIP3, GLYATL2, GLYATL3, GLYCTK, GLYR1, GM2A, GMCL1, GMDS, GMEB1, GMEB2, GMFB, GMFG, GMIP, GML, GMNC, GMNN, GMPPA, GMPPB, GMPR, GMPR2, GMPS, GNA11, GNA12, GNA13, GNA14, GNA15, GNAII, GNAI2, GNAI3, GNAL, GNAO1, GNAQ, GNAS, GNATI, GNAT2, GNAT3, GNAZ, GNB1, GNBIL, GNB2, GNB3, GNB4, GNB5, GNE, GNG10, GNG11, GNG12, GNG13, GNG14, GNG2, GNG3, GNG4, GNG5, GNG7, GNG8, GNGT1, GNGT2, GNLI, GNL2, GNL3, GNL3L, GNLY, GNMT, GNPAT, GNPDA1, GNPDA2, GNPNATI, GNPTAB, GNPTG, GNRH1, GNRH2, GNRHR, GNS, GOLGA1, GOLGA2, GOLGA3, GOLGA4, GOLGA5, GOLGA6A, GOLGA6B, GOLGA6C, GOLGA6D, GOLGA6L1, GOLGA6L10, GOLGA6L2, GOLGA6L22, GOLGA6L4, GOLGA6L6, GOLGA6L7P, GOLGA6L9, GOLGA7, GOLGA7B, GOLGA8A, GOLGA8B, GOLGA8F, GOLGA8G, GOLGA8H, GOLGA8J, GOLGA8K, GOLGA8M, GOLGA8N, GOLGA80, GOLGA8Q, GOLGA8R, GOLGA8S, GOLGAST, GOLGB1, GOLIM4, GOLM1, GOLPH3, GOLPH3L, GOLTIA, GOLTIB, GON4L, GON7, GOPC, GORAB, GORASP1, GORASP2, GOSRI, GOSR2, GOTI, GOTILI, GOT2, GPIBA, GPIBB, GP2, GP5, GP6, GP9, GPA33, GPAAI, GPALPP1, GPAM, GPANKI, GPAT2, GPAT3, GPAT4, GPATCHI, GPATCH11, GPATCH2, GPATCH2L, GPATCH3, GPATCH4, GPATCH8, GPBARI, GPBP1, GPBPIL1, GPC1, GPC2, GPC3, GPC4, GPC5, GPC6, GPCPD1, GPD1, GPDIL, GPD2, GPER1, GPHA2, GPHB5, GPHN, GPI, GPIHBP1, GPKOW, GPLD1, GPM6A, GPM6B, GPNI, GPN2, GPN3, GPNMB, GPR1, GPR101, GPR107, GPR108, GPR119, GPR12, GPR132, GPR135, GPR137, GPR137B, GPR137C, GPR139, GPR141, GPR142, GPR143, GPR146, GPR148, GPR149, GPR15, GPR150, GPR151, GPR152, GPR153, GPR155, GPR156, GPR157, GPR158, GPR160, GPR161, GPR162, GPR17, GPR171, GPR173, GPR174, GPR176, GPR179, GPR18, GPR180, GPR182, GPR183, GPR19, GPR20, GPR21, GPR22, GPR25, GPR26, GPR27, GPR3, GPR31, GPR32, GPR33, GPR34, GPR35, GPR37, GPR37L1, GPR39, GPR4, GPR42, GPR45, GPR50, GPR52, GPR55, GPR6, GPR61, GPR62, GPR63, GPR65, GPR68, GPR75, GPR75-ASB3, GPR78, GPR82, GPR83, GPR84, GPR85, GPR87, GPR88, GPR89A, GPR89B, GPRASP1, GPRASP2, GPRC5A, GPRC5B, GPRC5C, GPRC5D, GPRC6A, GPRIN1, GPRIN2, GPRIN3, GPS1, GPS2, GPSM1, GPSM2, GPSM3, GPT, GPT2, GPX1, GPX2, GPX3, GPX4, GPX5, GPX6, GPX7, GPX8, GRAMDIA, GRAMDIB, GRAMDIC, GRAMD2A, GRAMD2B, GRAMD4, GRAP, GRAP2, GRAPL, GRASP, GRB10, GRB14, GRB2, GRB7, GREB1, GREBIL, GREMI, GREM2, GRHLI, GRHL2, GRHL3, GRHPR, GRIAI, GRIA2, GRIA3, GRIA4, GRID1, GRID2, GRID2IP, GRIFIN, GRIK1, GRIK2, GRIK3, GRIK4, GRIK5, GRINI, GRIN2A, GRIN2B, GRIN2C, GRIN2D, GRIN3A, GRIN3B, GRINA, GRIP1, GRIP2, GRIPAP1, GR^{k1}, GR^{k2}, GR^{k3}, GR^{k4}, GR^{k5}, GR^{k6}, GR^{k7}, GRM1, GRM2, GRM3, GRM4, GRM5, GRM6, GRM7, GRM8, GRN, GRP, GRPEL1, GRPEL2, GRPR, GRSF1, GRTP1, GRWD1, GRXCRI, GRXCR2, GSAP, GSC, GSC2, GSDMA, GSDMB, GSDMC, GSDMD, GSE1, GSG1, GSGIL, GSG1L2, GSK3A, GSK3B, GSKIP, GSN, GSPT1, GSPT2, GSR, GSS, GSTA1, GSTA2, GSTA3, GSTA4, GSTA5, GSTCD, GSTKI, GSTMI, GSTM2, GSTM3, GSTM4, GSTM5, GSTO1, GSTO2, GSTP1, GSTTI, GSTT2, GSTT2B, GSTTP1, GSTZ1, GSX1, GSX2, GTDC1, GTF2A1, GTF2A1L, GTF2A2, GTF2B, GTF2E1, GTF2E2, GTF2F1, GTF2F2, GTF2H1, GTF2H2, GTF2H2C, GTF2H2C_2, GTF2H3, GTF2H4, GTF2H5, GTF2I, GTF2IRD1, GTF2IRD2, GTF2IRD2B, GTF3A, GTF3C1, GTF3C2, GTF3C3, GTF3C4, GTF3C5, GTF3C6, GTPBP1, GTPBP10, GTPBP2, GTPBP3, GTPBP4, GTPBP6, GTPBP8, GTSE1, GTSF1, GTSFIL, GU182339.1, GU182339.3, GU182343.1, GU182343.2, GU182345.1, GU182345.2, GU182347.1, GU182351.2, GU182352.2, GU182353.1, GU182355.1, GU182355.2, GU182355.3, GU182357.1, GU182357.3, GU182359.1, GU182359.2, GUCAIA, GUCAIB, GUCAIC, GUCA2A, GUCA2B, GUCD1, GUCY1A2, GUCY1A3, GUCY1B3, GUCY2C, GUCY2D, GUCY2F, GUFI, GUK1, GULP1, GUSB, GVQW2, GXYLT1, GXYLT2, GYG1, GYG2, GYPA, GYPB, GYPC, GYPE, GYS1, GYS2, GZF1, GZMA, GZMB, GZMH, GZMK, GZMM, H1F0, HIFNT, HIFOO, H1FX, H2AFB1, H2AFB2, H2AFB3, H2AFJ, H2AFV, H2AFX, H2AFY, H2AFY2, H2AFZ, H2BFM, H2BFS, H2BFWT, H3F3A, H3F3B, H3F3C, H6PD, HAAO, HABP2, HABP4, HACD1, HACD2, HACD3, HACD4, HACE1, HACLI, HADH, HADHA, HADHB, HAGH, HAGHL, HAL, HAMP, HAND1, HAND2, HA01, HAO2, HAP1, HAPLN1, HAPLN2, HAPLN3, HAPLN4, HARBI1, HARS, HARS2, HAS1, HAS2, HAS3, HASPIN, HAT1, HAUS1, HAUS2, HAUS3, HAUS4, HAUS5, HAUS6, HAUS7, HAUS8, HAVCRI, HAVCR2, HAX1, HBA1, HBA2, HBB, HBD, HBE1, HBEGF, HBG1, HBG2, HBM, HBP1, HBQ1, HBSIL, HBZ, HCAR1, HCAR2, HCAR3, HCCS, HCFC1, HCFCIR1, HCFC2, HCK, HCLS1, HCN1, HCN2, HCN3, HCN4, HCRT, HCRTRI, HCRTR2, HCST, HDAC1, HDAC10, HDAC11, HDAC2, HDAC3, HDAC4, HDAC5, HDAC6, HDAC7, HDAC8, HDAC9, HDC, HDDC2, HDDC3, HDGF, HDGFL1, HDGFL2, HDGFL3, HDHD2, HDHD3, HDHD5, HDLBP, HDX, HEATR1, HEATR3, HEATR4, HEATRSA, HEATR5B, HEATR6, HEATR9, HEBP1, HEBP2, HECA, HECTDI, HECTD2, HECTD3, HECTD4, HECW1, HECW2, HEGI, HELB, HELLS, HELQ, HELT, HELZ, HELZ2, HEMGN, HEMK1, HENMTI, HEPACAM, HEPACAM2, HEPH, HEPHL1, HEPN1, HERCI, HERC2, HERC3, HERC4, HERC5, HERC6, HERPUD1, HERPUD2, HES1, HES2, HES3, HES4, HES5, HES6, HES7, HESX1, HEXA, HEXB, HEXDC, HEXIMI, HEXIM2, HEY1, HEY2, HEYL, HFE, HFE2, HFM1, HGD, HGF, HGFAC, HGHI, HGNC: 18790, HGNC: 24955, HGS, HGSNAT, HHAT, HHATL, HHEX, HHIP, HHIPL1, HHIPL2, HHLA1, HHLA2, HHLA3, HIBADH, HIBCH, HICI, HIC2, HIDI, HIFIA, HIFIAN, HIF3A, HIGDIA, HIGDIB, HIGDIC, HIGD2A, HIGD2B, HIKESHI, HILPDA, HINFP, HINT1, HINT2, HINT3, HIP1, HIPIR, HIPK1, HIPK2, HIPK3, HIPK4, HIR^{A}, HIRIP3, HISTIHIA, HISTIHIB, HISTIHIC, HISTIHID, HISTIHIE, HISTIHIT, HISTIH2AA, HISTIH2AB, HISTIH2AC, HISTIH2AD, HISTIH2AE, HISTIH2AG, HIST1H2AH, HISTIH2AI, HISTIH2AJ, HISTIH2AK, HISTIH2AL, HISTIH2AM, HISTIH2BA, HIST1H2BB, HISTIH2BC, HIST1H2BD, HISTIH2BE, HISTIH2BF, HISTIH2BG, HIST1H2BH, HISTIH2BI, HISTIH2BJ, HISTIH2BK, HISTIH2BL, HISTIH2BM, HISTIH2BN, HISTIH2BO, HIST1H3A, HISTIH3B, HISTIH3C, HISTIH3D, HISTIH3E, HISTIH3F, HISTIH3G, HISTIH3H, HIST1H3I, HISTIH3J, HISTIH4A, HISTIH4B, HISTIH4C, HISTIH4D, HISTIH4E, HISTIH4F, HIST1H4G, HISTIH4H, HISTIH4I, HIST1H4J, HISTIH4K, HISTIH4L, HIST2H2AA3, HIST2H2AA4, HIST2H2AB, HIST2H2AC, HIST2H2BE, HIST2H2BF, HIST2H3A, HIST2H3C, HIST2H3D, HIST2H3PS2, HIST2H4A, HIST2H4B, HIST3H2A, HIST3H2BB, HIST3H3, HIST4H4, HIVEP1, HIVEP2, HIVEP3, HJURP, HK1, HK2, HK3, HKDC1, HKRI, HLA-A, HLA-B, HLA-C, HLA-DMA, HLA-DMB, HLA-DOA, HLA-DOB, HLA-DPA1, HLA-DPB1, HLA-DQA1, HLA-DQA2, HLA-DQB1, HLA-DQB2, HLA-DR^{A}, HLA-DRB1, HLA-DRB3, HLA-DRB4, HLA-DRB5, HLA-E, HLA-F, HLA-G, HLCS, HLF, HLTF, HLX, HM13, HM190170.1, HMBOX1, HMBS, HMCES, HMCNI, HMCN2, HMG20A, HMG20B, HMGA1, HMGA2, HMGB1, HMGB2, HMGB3, HMGB4, HMGCL, HMGCLL1, HMGCR, HMGCS1, HMGCS2, HMGN1, HMGN2, HMGN3, HMGN4, HMGN5, HMGXB3, HMGXB4, HMHB1, HMMR, HMOX1, HMOX2, HMSD, HMX1, HMX2, HMX3, HNFIA, HNF1B, HNF4A, HNF4G, HNMT, HNRNPAO, HNRNPA1, HNRNPAIL2, HNRNPA2B1, HNRNPA3, HNRNPAB, HNRNPC, HNRNPCL1, HNRNPCL2, HNRNPCL3, HNRNPCL4, HNRNPD, HNRNPDL, HNRNPF, HNRNPH1, HNRNPH2, HNRNPH3, HNRNPK, HNRNPL, HNRNPLL, HNRNPM, HNRNPR, HNRNPU, HNRNPULI, HNRNPUL2, HNRNPUL2-BSCL2, HOGAI, HOMERI, HOMER2, HOMER3, HOMEZ, HOOK1, HOOK2, HOOK3, HOPX, HORMAD1, HORMAD2, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HP, HP1BP3, HPCA, HPCALI, HPCAL4, HPD, HPDL, HPF1, HPGD, HPGDS, HPN, HPR, HPRTI, HPS1, HPS3, HPS4, HPS5, HPS6, HPSE, HPSE2, HPX, HR, HRAS, HRASLS, HRASLS2, HRASLS5, HRC, HRCT1, HRG, HRH1, HRH2, HRH3, HRH4, HRK, HRNR, HS1BP3, HS2ST1, HS3ST1, HS3ST2, HS3ST3A1, HS3ST3B1, HS3ST4, HS3ST5, HS3ST6, HS6ST1, HS6ST2, HS6ST3, HSBP1, HSBPIL1, HSCB, HSD11B1, HSD11BIL, HSD11B2, HSD17B1, HSD17B10, HSD17B11, HSD17B12, HSD17B13, HSD17B14, HSD17B2, HSD17B3, HSD17B4, HSD17B6, HSD17B7, HSD17B8, HSD3B1, HSD3B2, HSD3B7, HSDLI, HSDL2, HSF1, HSF2, HSF2BP, HSF4, HSF5, HSFX1, HSFX2, HSFX3, HSFX4, HSFY1, HSFY2, HSH2D, HSP90AA1, HSP90AB1, HSP90B1, HSPA12A, HSPA12B, HSPA13, HSPA14, HSPAIA, HSPAIB, HSPAIL, HSPA2, HSPA4, HSPA4L, HSPA5, HSPA6, HSPA8, HSPA9, HSPB1, HSPB11, HSPB2, HSPB2-C11orf52, HSPB3, HSPB6, HSPB7, HSPB8, HSPB9, HSPBAP1, HSPBP1, HSPD1, HSPE1, HSPE1-MOB4, HSPG2, HSPH1, HTATIP2, HTATSF1, HTD2, HTN1, HTN3, HTRIA, HTRIB, HTRID, HTR1E, HTRIF, HTR2A, HTR2B, HTR2C, HTR3A, HTR3B, HTR3C, HTR3D, HTR3E, HTR4, HTR5A, HTR6, HTR7, HTRAI, HTRA2, HTRA3, HTRA4, HTT, HUNK, HUSI, HUSIB, HUWEI, HVCNI, HYALI, HYAL2, HYAL3, HYAL4, HYDIN, HYI, HYKK, HYLS1, HYOUI, HYPK, HYPM, IAHI, IAPP, IARS, IARS2, IBA57, IBSP, IBTK, ICA1, ICAIL, ICAMI, ICAM2, ICAM3, ICAM4, ICAM5, ICE1, ICE2, ICK, ICMT, ICOS, ICOSLG, ID1, ID2, ID3, ID4, IDE, IDH1, IDH2, IDH3A, IDH3B, IDH3G, IDI1, IDI2, IDNK, IDO1, IDO2, IDS, IDUA, IER2, IER3, IER3IP1, IER5, IER5L, IFFO1, IFFO2, IFI16, IFI27, IFI27L1, IFI27L2, IFI30, IFI35, IFI44, IFI44L, IFI6, IFIH1, IFIT1, IFIT1B, IFIT2, IFIT3, IFIT5, IFITM1, IFITM10, IFITM2, IFITM3, IFITM5, IFNA1, IFNA10, IFNA13, IFNA14, IFNA16, IFNA17, IFNA2, IFNA21, IFNA4, IFNA5, IFNA6, IFNA7, IFNA8, IFNARI, IFNAR2, IFNB1, IFNE, IFNG, IFNGR1, IFNGR2, IFNK, IFNL1, IFNL2, IFNL3, IFNL4, IFNLR1, IFNW1, IFRD1, IFRD2, IFT122, IFT140, IFT172, IFT20, IFT22, IFT27, IFT43, IFT46, IFT52, IFT57, IFT74, IFT80, IFT81, IFT88, IGBP1, IGDCC3, IGDCC4, IGF1, IGFIR, IGF2, IGF2BP1, IGF2BP2, IGF2BP3, IGF2R, IGFALS, IGFBP1, IGFBP2, IGFBP3, IGFBP4, IGFBP5, IGFBP6, IGFBP7, IGFBPL1, IGFLI, IGFL2, IGFL3, IGFL4, IGFLR1, IGFNI, IGHA1, IGHA2, IGHD, IGHD1-1, IGHD1-14, IGHD1-20, IGHD1-26, IGHD1-7, IGHD1OR15-1A, IGHD1OR15-1B, IGHD2-15, IGHD2-2, IGHD2-21, IGHD2-8, IGHD2OR15-2A, IGHD2OR15-2B, IGHD3-10, IGHD3-16, IGHD3-22, IGHD3-3, IGHD3-9, IGHD3OR15-3A, IGHD3OR15-3B, IGHD4-11, IGHD4-17, IGHD4-23, IGHD4-4, IGHD4OR15-4A, IGHD4OR15-4B, IGHD5-12, IGHD5-18, IGHD5-24, IGHD5-5, IGHD5OR15-5A, IGHD5OR15-5B, IGHD6-13, IGHD6-19, IGHD6-25, IGHD6-6, IGHD7-27, IGHE, IGHG1, IGHG2, IGHG3, IGHG4, IGHJI, IGHJ2, IGHJ3, IGHJ4, IGHJ5, IGHJ6, IGHM, IGHMBP2, IGHV1-18, IGHV1-2, IGHV1-24, IGHV1-3, IGHV1-45, IGHV1-46, IGHV1-58, IGHV1-69, IGHV1OR15-1, IGHVIOR15-9, IGHV1OR21-1, IGHV2-26, IGHV2-5, IGHV2-70, IGHV2OR16-5, IGHV3-11, IGHV3-13, IGHV3-15, IGHV3-16, IGHV3-20, IGHV3-21, IGHV3-23, IGHV3-30, IGHV3-33, IGHV3-35, IGHV3-38, IGHV3-43, IGHV3-48, IGHV3-49, IGHV3-53, IGHV3-64, IGHV3-66, IGHV3-7, IGHV3-72, IGHV3-73, IGHV3-74, IGHV3OR15-7, IGHV3OR16-10, IGHV3OR16-12, IGHV3OR16-13, IGHV3OR16-8, IGHV3OR16-9, IGHV4-28, IGHV4-31, IGHV4-34, IGHV4-39, IGHV4-4, IGHV4-59, IGHV4-61, IGHV4OR15-8, IGHV5-51, IGHV6-1, IGHV7-81, IGIP, IGKC, IGKJI, IGKJ2, IGKJ3, IGKJ4, IGKJ5, IGKV1-12, IGKV1-16, IGKV1-17, IGKV1-27, IGKV1-33, IGKV1-37, IGKV1-39, IGKV1-5, IGKV1-6, IGKV1-8, IGKV1-9, IGKVID-12, IGKVID-13, IGKVID-16, IGKVID-17, IGKVID-33, IGKVID-37, IGKVID-39, IGKVID-42, IGKVID-43, IGKVID-8, IGKV1OR2-108, IGKV2-24, IGKV2-28, IGKV2-30, IGKV2-40, IGKV2D-24, IGKV2D-26, IGKV2D-28, IGKV2D-29, IGKV2D-30, IGKV2D-40, IGKV3-11, IGKV3-15, IGKV3-20, IGKV3-7, IGKV3D-11, IGKV3D-15, IGKV3D-20, IGKV3D-7, IGKV3OR2-268, IGKV4-1, IGKV5-2, IGKV6-21, IGKV6D-21, IGKV6D-41, IGLC1, IGLC2, IGLC3, IGLC7, IGLJI, IGLJ2, IGLJ3, IGLJ4, IGLJ5, IGLJ6, IGLJ7, IGLLI, IGLL5, IGLON5, IGLV10-54, IGLV11-55, IGLV1-36, IGLV1-40, IGLV1-44, IGLV1-47, IGLV1-50, IGLV1-51, IGLV2-11, IGLV2-14, IGLV2-18, IGLV2-23, IGLV2-33, IGLV2-8, IGLV3-1, IGLV3-10, IGLV3-12, IGLV3-16, IGLV3-19, IGLV3-21, IGLV3-22, IGLV3-25, IGLV3-27, IGLV3-32, IGLV3-9, IGLV4-3, IGLV4-60, IGLV4-69, IGLV5-37, IGLV5-45, IGLV5-48, IGLV5-52, IGLV6-57, IGLV7-43, IGLV7-46, IGLV8-61, IGLV9-49, IGSF1, IGSF10, IGSF11, IGSF21, IGSF22, IGSF23, IGSF3, IGSF5, IGSF6, IGSF8, IGSF9, IGSF9B, IHH, IK, IKBIP, IKBKB, IKBKE, IKBKG, IKZF1, IKZF2, IKZF3, IKZF4, IKZF5, IL10, IL1OR^{A}, IL10RB, IL11, IL11R^{A}, IL12A, IL12B, IL12RB1, IL12RB2, IL13, IL13RA1, IL13RA2, IL15, IL15R^{A}, IL16, IL17A, IL17B, IL17C, IL17D, IL17F, IL17R^{A}, IL17RB, IL17RC, IL17RD, IL17RE, IL17REL, IL18, IL18BP, IL18R¹, IL18RAP, IL19, ILIA, ILIB, IL1F10, ILIR1, ILIR2, IL1RAP, ILIRAPLI, ILIRAPL2, ILIRL1, ILIRL2, ILIRN, IL2, IL20, IL20R^{A}, IL20RB, IL21, IL21R, IL22, IL22RA1, IL22RA2, IL23A, IL23R, IL24, IL25, IL26, IL27, IL27R^{A}, IL2R^{A}, IL2RB, IL2RG, IL3, IL31, IL31R^{A}, IL32, IL33, IL34, IL36A, IL36B, IL36G, IL36RN, IL37, IL3R^{A}, IL4, IL4I1, IL4R, IL5, IL5R^{A}, IL6, IL6R, IL6ST, IL7, IL7R, IL9, IL9R, ILDRI, ILDR2, ILF2, ILF3, ILK, ILKAP, ILVBL, IMMPIL, IMMP2L, IMMT, IMP3, IMP4, IMPA1, IMPA2, IMPACT, IMPAD1, IMPDH1, IMPDH2, IMPG1, IMPG2, INA, INAFM1, INAFM2, INAVA, INCA1, INCENP, INF2, ING1, ING2, ING3, ING4, ING5, INHA, INHBA, INHBB, INHBC, INHBE, INIP, INMT, INMT-MINDY4, INO80, INO80B, INO80B-WBP1, INO80C, INO80D, INO80E, INPP1, INPP4A, INPP4B, INPP5A, INPP5B, INPP5D, INPP5E, INPP5F, INPP5J, INPP5K, INPPLI, INS, INSC, INSIG1, INSIG2, INS-IGF2, INSL3, INSL4, INSL5, INSL6, INSMI, INSM2, INSR, INSRR, INTSI, INTS10, INTS11, INTS12, INTS13, INTS14, INTS2, INTS3, INTS4, INTS5, INTS6, INTS6L, INTS7, INTS8, INTS9, INTU, INVS, IP6K1, IP6K2, IP6K3, IPCEF1, IPMK, IPO11, IPO13, IPO4, IPO5, IPO7, IPO8, IPO9, IPP, IPPK, IQANKI, IQCA1, IQCAIL, IQCB1, IQCC, IQCD, IQCE, IQCF1, IQCF2, IQCF3, IQCF5, IQCF6, IQCG, IQCH, IQCJ, IQCJ-SCHIP1, IQCK, IQCM, IQGAPI, IQGAP2, IQGAP3, IQSECI, IQSEC2, IQSEC3, IQUB, IREB2, IRF1, IRF2, IRF2BP1, IRF2BP2, IRF2BPL, IRF3, IRF4, IRF5, IRF6, IRF7, IRF8, IRF9, IRGC, IRGM, IRGQ, IRS1, IRS2, IRS4, IRX1, IRX2, IRX3, IRX4, IRX5, IRX6, ISCA1, ISCA2, ISCU, ISG15, ISG20, ISG20L2, ISL1, ISL2, ISLR, ISLR2, ISMI, ISM2, ISOC1, ISOC2, ISPD, IST1, ISX, ISY1, ISY1-RAB43, ISYNA1, ITCH, ITFG1, ITFG2, ITGAI, ITGA10, ITGA11, ITGA2, ITGA2B, ITGA3, ITGA4, ITGA5, ITGA6, ITGA7, ITGA8, ITGA9, ITGAD, ITGAE, ITGAL, ITGAM, ITGAV, ITGAX, ITGB1, ITGB1BP1, ITGB1BP2, ITGB2, ITGB3, ITGB3BP, ITGB4, ITGB5, ITGB6, ITGB7, ITGB8, ITGBLI, ITIHI, ITIH2, ITIH3, ITIH4, ITIH5, ITIH6, ITK, ITLNI, ITLN2, ITM2A, ITM2B, ITM2C, ITPA, ITPKI, ITPKA, ITPKB, ITPKC, ITPRI, ITPR2, ITPR3, ITPRIP, ITPRIPLI, ITPRIPL2, ITSN1, ITSN2, IVD, IVL, IVNSIABP, IWS1, IYD, IZUMO1, IZUMOIR, IZUMO2, IZUMO3, IZUMO4, JADE1, JADE2, JADE3, JAG1, JAG2, JAGNI, JAKI, JAK2, JAK3, JAKMIPI, JAKMIP2, JAKMIP3, JAM2, JAM3, JAML, JARID2, JAZF1, JCAD, JCHAIN, JDP2, JKAMP, JMJDIC, JMJD4, JMJD6, JMJD7, JMJD7-PLA2G4B, JMJD8, JMY, JOSDI, JOSD2, JPHI, JPH2, JPH3, JPH4, JPT1, JPT2, JRK, JRKL, JSRP1, JTB, JUN, JUNB, JUND, JUP, KAAG1, KALRN, KANKI, KANK2, KANK3, KANK4, KANSLI, KANSLIL, KANSL2, KANSL3, KANTR, KARS, KAT14, KAT2A, KAT2B, KAT5, KAT6A, KAT6B, KAT7, KAT8, KATNA1, KATNALI, KATNAL2, KATNB1, KATNBLI, KAZALDI, KAZN, KBTBD11, KBTBD11-OT1, KBTBD12, KBTBD13, KBTBD2, KBTBD3, KBTBD4, KBTBD6, KBTBD7, KBTBD8, KCMF1, KCNA1, KCNA10, KCNA2, KCNA3, KCNA4, KCNA5, KCNA7, KCNAB1, KCNAB2, KCNAB3, KCNB1, KCNB2, KCNC1, KCNC2, KCNC3, KCNC4, KCND1, KCND2, KCND3, KCNE1, KCNEIB, KCNE2, KCNE3, KCNE4, KCNE5, KCNF1, KCNG1, KCNG2, KCNG3, KCNG4, KCNH1, KCNH2, KCNH3, KCNH4, KCNH5, KCNH6, KCNH7, KCNH8, KCNIP1, KCNIP2, KCNIP3, KCNIP4, KCNJI, KCNJ10, KCNJ11, KCNJ12, KCNJ13, KCNJ14, KCNJ15, KCNJ16, KCNJ18, KCNJ2, KCNJ3, KCNJ4, KCNJ5, KCNJ6, KCNJ8, KCNJ9, KCNK1, KCNK10, KCNK12, KCNK13, KCNK15, KCNK16, KCNK17, KCNK18, KCNK2, KCNK3, KCNK4, KCNK5, KCNK6, KCNK7, KCNK9, KCNMA1, KCNMB1, KCNMB2, KCNMB3, KCNMB4, KCNN1, KCNN2, KCNN3, KCNN4, KCNQ1, KCNQ2, KCNQ3, KCNQ4, KCNQ5, KCNRG, KCNSI, KCNS2, KCNS3, KCNT1, KCNT2, KCNU1, KCNVI, KCNV2, KCP, KCTD1, KCTD10, KCTD11, KCTD12, KCTD13, KCTD14, KCTD15, KCTD16, KCTD17, KCTD18, KCTD19, KCTD2, KCTD20, KCTD21, KCTD3, KCTD4, KCTD5, KCTD6, KCTD7, KCTD8, KCTD9, KDELCI, KDELC2, KDELRI, KDELR2, KDELR3, KDF1, KDMIA, KDMIB, KDM2A, KDM2B, KDM3A, KDM3B, KDM4A, KDM4B, KDM4C, KDM4D, KDM4E, KDM4F, KDM5A, KDM5B, KDM5C, KDM5D, KDM6A, KDM6B, KDM7A, KDM8, KDR, KDSR, KEAPI, KEL, KER^{A}, KF459570.1, KHDC1, KHDCIL, KHDC3L, KHDRBS1, KHDRBS2, KHDRBS3, KHK, KHNYN, KHSRP, KIAA0040, KIAA0100, KIAA0141, KIAA0232, KIAA0319, KIAA0319L, KIAA0355, KIAA0368, KIAA0391, KIAA0408, KIAA0513, KIAA0556, KIAA0586, KIAA0753, KIAA0825, KIAA0895, KIAA0895L, KIAA0907, KIAA0930, KIAA1024, KIAA1024L, KIAA1107, KIAA1109, KIAA1143, KIAA1147, KIAA1161, KIAA1191, KIAA1210, KIAA1211, KIAA1211L, KIAA1217, KIAA1257, KIAA1324, KIAA1324L, KIAA1328, KIAA1456, KIAA1468, KIAA1522, KIAA1524, KIAA1549, KIAA1549L, KIAA1551, KIAA1586, KIAA1614, KIAA1644, KIAA1671, KIAA1683, KIAA1755, KIAA1841, KIAA1958, KIAA2012, KIAA2013, KIAA2026, KIDINS220, KIF11, KIF12, KIF13A, KIF13B, KIF14, KIF15, KIF16B, KIF17, KIF18A, KIF18B, KIF19, KIFIA, KIFIB, KIF1BP, KIFIC, KIF20A, KIF20B, KIF21A, KIF21B, KIF22, KIF23, KIF24, KIF25, KIF26A, KIF26B, KIF27, KIF2A, KIF2B, KIF2C, KIF3A, KIF3B, KIF3C, KIF4A, KIF4B, KIF5A, KIF5B, KIF5C, KIF6, KIF7, KIF9, KIFAP3, KIFCI, KIFC2, KIFC3, KIN, KIR2DL1, KIR2DL2, KIR2DL3, KIR2DL4, KIR2DL5A, KIR2DL5B, KIR2DP1, KIR2DS1, KIR2DS2, KIR2DS3, KIR2DS4, KIR2DS5, KIR3DL1, KIR3DL2, KIR3DL3, KIR3DP1, KIR3DS1, KIR3DX1, KIRRELI, KIRREL2, KIRREL3, KISS1, KISSIR, KIT, KITLG, KIZ, KL, KLB, KLC1, KLC2, KLC3, KLC4, KLF1, KLF10, KLF11, KLF12, KLF13, KLF14, KLF15, KLF16, KLF17, KLF18, KLF2, KLF3, KLF4, KLF5, KLF6, KLF7, KLF8, KLF9, KLHDC1, KLHDC10, KLHDC2, KLHDC3, KLHDC4, KLHDC7A, KLHDC7B, KLHDC8A, KLHDC8B, KLHDC9, KLHLI, KLHL10, KLHL11, KLHL12, KLHL13, KLHL14, KLHL15, KLHL17, KLHL18, KLHL2, KLHL20, KLHL21, KLHL22, KLHL23, KLHL24, KLHL25, KLHL26, KLHL28, KLHL29, KLHL3, KLHL30, KLHL31, KLHL32, KLHL33, KLHL34, KLHL35, KLHL36, KLHL38, KLHL4, KLHL40, KLHL41, KLHL42, KLHL5, KLHL6, KLHL7, KLHL8, KLHL9, KLK1, KLK10, KLK11, KLK12, KLK13, KLK14, KLK15, KLK2, KLK3, KLK4, KLK5, KLK6, KLK7, KLK8, KLK9, KLKB1, KLLN, KLRB1, KLRC1, KLRC2, KLRC3, KLRC4, KLRC4-KLR^{k1}, KLRD1, KLRF1, KLRF2, KLRG1, KLRG2, KLR^{k1}, KMO, KMT2A, KMT2B, KMT2C, KMT2D, KMT2E, KMT5A, KMT5B, KMT5C, KNCN, KNDC1, KNG1, KNL1, KNOP1, KNSTRN, KNTC1, KP420437.1, KP420437.2, KP420437.3, KP420439.1, KP420440.1, KP420440.2, KP420440.3, KP420440.4, KP420440.5, KP420440.6, KP420440.7, KP420440.8, KP420440.9, KP420441.1, KP420441.2, KP420441.3, KP420441.4, KP420441.5, KP420442.2, KP420442.3, KP420443.1, KP420444.1, KP420444.2, KP420444.3, KP420444.4, KP420444.5, KP420444.6, KP420444.7, KP420446.1, KP420446.2, KPNA1, KPNA2, KPNA3, KPNA4, KPNA5, KPNA6, KPNA7, KPNB1, KPRP, KPTN, KRAS, KRBA1, KRBA2, KRBOX1, KRBOX4, KRCC1, KREMEN1, KREMEN2, KRI1, KRITI, KRRI, KR^{T1}, KR^{T10}, KR^{T12}, KR^{T13}, KR^{T14}, KR^{T15}, KR^{T16}, KR^{T17}, KR^{T18}, KR^{T19}, KR^{T2}, KR^{T20}, KR^{T222}, KR^{T23}, KR^{T24}, KR^{T25}, KR^{T26}, KR^{T27}, KR^{T28}, KR^{T3}, KR^{T31}, KR^{T32}, KR^{T33}A, KR^{T33}B, KR^{T34}, KR^{T35}, KR^{T36}, KR^{T37}, KR^{T38}, KR^{T39}, KR^{T4}, KR^{T40}, KR^{T5}, KR^{T6}A, KR^{T6}B, KR^{T6}C, KR^{T7}, KR^{T71}, KR^{T72}, KR^{T73}, KR^{T74}, KR^{T75}, KR^{T76}, KR^{T77}, KR^{T78}, KR^{T79}, KR^{T8}, KR^{T80}, KR^{T81}, KR^{T82}, KR^{T83}, KR^{T84}, KR^{T85}, KR^{T86}, KR^{T9}, KRTAP10-1, KRTAP10-10, KRTAP10-11, KRTAP10-12, KRTAP10-2, KRTAP10-3, KRTAP10-4, KRTAP10-5, KRTAP10-6, KRTAP10-7, KRTAP10-8, KRTAP10-9, KRTAP1-1, KRTAP11-1, KRTAP12-1, KRTAP12-2, KRTAP12-3, KRTAP12-4, KRTAP1-3, KRTAP13-1, KRTAP13-2, KRTAP13-3, KRTAP13-4, KRTAP1-4, KRTAP1-5, KRTAP15-1, KRTAP16-1, KRTAP17-1, KRTAP19-1, KRTAP19-2, KRTAP19-3, KRTAP19-4, KRTAP19-5, KRTAP19-6, KRTAP19-7, KRTAP19-8, KRTAP20-1, KRTAP20-2, KRTAP20-3, KRTAP20-4, KRTAP2-1, KRTAP21-1, KRTAP21-2, KRTAP21-3, KRTAP2-2, KRTAP22-1, KRTAP22-2, KRTAP2-3, KRTAP23-1, KRTAP2-4, KRTAP24-1, KRTAP25-1, KRTAP26-1, KRTAP27-1, KRTAP29-1, KRTAP3-1, KRTAP3-2, KRTAP3-3, KRTAP4-1, KRTAP4-11, KRTAP4-12, KRTAP4-16, KRTAP4-2, KRTAP4-3, KRTAP4-4, KRTAP4-5, KRTAP4-6, KRTAP4-7, KRTAP4-8, KRTAP4-9, KRTAP5-1, KRTAP5-10, KRTAP5-11, KRTAP5-2, KRTAP5-3, KRTAP5-4, KRTAP5-5, KRTAP5-6, KRTAP5-7, KRTAP5-8, KRTAP5-9, KRTAP6-1, KRTAP6-2, KRTAP6-3, KRTAP7-1, KRTAP8-1, KRTAP9-1, KRTAP9-2, KRTAP9-3, KRTAP9-4, KRTAP9-6, KRTAP9-7, KRTAP9-8, KRTAP9-9, KRTCAP2, KRTCAP3, KRTDAP, KSR1, KSR2, KTI12, KTN1, KU645196.1, KU645196.2, KU645196.3, KU645196.4, KU645196.5, KU645196.6, KU645196.7, KU645196.8, KU645196.9, KU645197.1, KU645197.2, KU645197.3, KU645197.4, KU645197.5, KU645198.1, KXD1, KY, KYATI, KYAT3, KYNU, LICAM, LITD1, L2HGDH, L34079.1, L3HYPDH, L3MBTL1, L3MBTL2, L3MBTL3, L3MBTL4, LACCI, LACRT, LACTB, LACTB2, LACTBLI, LADI, LAG3, LAGE3, LAIRI, LAIR2, LALBA, LAMA1, LAMA2, LAMA3, LAMA4, LAMA5, LAMB1, LAMB2, LAMB3, LAMB4, LAMC1, LAMC2, LAMC3, LAMPI, LAMP2, LAMP3, LAMP5, LAMTORI, LAMTOR2, LAMTOR3, LAMTOR4, LAMTOR5, LANCLI, LANCL2, LANCL3, LAP3, LAPTM4A, LAPTM4B, LAPTM5, LARGE1, LARGE2, LARP1, LARPIB, LARP4, LARP4B, LARP6, LARP7, LARS, LARS2, LASIL, LASPI, LAT, LAT2, LATS1, LATS2, LAXI, LAYN, LBH, LBHD1, LBP, LBR, LBX1, LBX2, LCA5, LCA5L, LCAT, LCEIA, LCEIB, LCEIC, LCEID, LCEIE, LCEIF, LCE2A, LCE2B, LCE2C, LCE2D, LCE3A, LCE3B, LCE3C, LCE3D, LCE3E, LCE4A, LCE5A, LCE6A, LCK, LCLATI, LCMT1, LCMT2, LCN1, LCN10, LCN12, LCN15, LCN2, LCN6, LCN8, LCN9, LCNL1, LCOR, LCORL, LCP1, LCP2, LCT, LCTL, LDAH, LDB1, LDB2, LDB3, LDHA, LDHAL6A, LDHAL6B, LDHB, LDHC, LDHD, LDLR, LDLRADI, LDLRAD2, LDLRAD3, LDLRAD4, LDLRAP1, LDOC1, LEAP2, LECT2, LEF1, LEFTY1, LEFTY2, LEKRI, LELP1, LEMD1, LEMD2, LEMD3, LENEP, LENG1, LENG8, LENG9, LEO1, LEP, LEPR, LEPROT, LEPROTLI, LETMI, LETM2, LETMD1, LEUTX, LEXM, LFNG, LGALS1, LGALS12, LGALS13, LGALS14, LGALS16, LGALS2, LGALS3, LGALS3BP, LGALS4, LGALS7, LGALS7B, LGALS8, LGALS9, LGALS9B, LGALS9C, LGALSL, LGI1, LGI2, LGI3, LGI4, LGMN, LGR4, LGR5, LGR6, LGSN, LHB, LHCGR, LHFPLI, LHFPL2, LHFPL3, LHFPL4, LHFPL5, LHFPL6, LHPP, LHX1, LHX2, LHX3, LHX4, LHX5, LHX6, LHX8, LHX9, LIAS, LIF, LIFR, LIGI, LIG3, LIG4, LILRA1, LILRA2, LILRA3, LILRA4, LILRA5, LILRA6, LILRBI, LILRB2, LILRB3, LILRB4, LILRB5, LIM2, LIMA1, LIMCHI, LIMDI, LIMD2, LIME1, LIMKI, LIMK2, LIMS1, LIMS2, LIMS3, LIMS4, LIN28A, LIN28B, LIN37, LIN52, LIN54, LIN7A, LIN7B, LIN7C, LIN9, LINC00094, LINC00116, LINC00282, LINC00672, LINC00675, LINC00694, LINC00854, LINC00890, LINC00959, LINC01125, LINC01556, LINC02210-CRHRI, LINGO1, LINGO2, LINGO3, LINGO4, LINSI, LIPA, LIPC, LIPE, LIPF, LIPG, LIPH, LIPI, LIPJ, LIPK, LIPM, LIPN, LIPTI, LIPT2, LITAF, LIX1, LIXIL, LKAAEARI, LLGLI, LLGL2, LLPH, LMANI, LMANIL, LMAN2, LMAN2L, LMBRI, LMBRIL, LMBRD1, LMBRD2, LMCD1, LMF1, LMF2, LMLN, LMNA, LMNB1, LMNB2, LMNTD1, LMNTD2, LM01, LMO2, LM03, LM04, LM07, LMO7DN, LMOD1, LMOD2, LMOD3, LMTK2, LMTK3, LMXIA, LMX1B, LNP1, LNPEP, LNPK, LNX1, LNX2, LO000005.1, LONP1, LONP2, LONRF1, LONRF2, LONRF3, LOR, LOX, LOXHD1, LOXLI, LOXL2, LOXL3, LOXL4, LPA, LPARI, LPAR2, LPAR3, LPAR4, LPAR5, LPAR6, LPCATI, LPCAT2, LPCAT3, LPCAT4, LPGATI, LPINI, LPIN2, LPIN3, LPL, LPO, LPP, LPXN, LRAT, LRBA, LRCHI, LRCH2, LRCH3, LRCH4, LRCOL1, LRFN1, LRFN2, LRFN3, LRFN4, LRFN5, LRG1, LRGUK, LRIFI, LRIGI, LRIG2, LRIG3, LRITI, LRIT2, LRIT3, LRMDA, LRMP, LRP1, LRP10, LRP11, LRP12, LRPIB, LRP2, LRP2BP, LRP3, LRP4, LRP5, LRP5L, LRP6, LRP8, LRPAP1, LRPPRC, LRRI, LRRCI, LRRC10, LRRC10B, LRRC14, LRRC14B, LRRC15, LRRC17, LRRC18, LRRC19, LRRC2, LRRC20, LRRC23, LRRC24, LRRC25, LRRC26, LRRC27, LRRC28, LRRC29, LRRC3, LRRC30, LRRC31, LRRC32, LRRC34, LRRC36, LRRC37A, LRRC37A2, LRRC37A3, LRRC37B, LRRC38, LRRC39, LRRC3B, LRRC3C, LRRC4, LRRC40, LRRC41, LRRC42, LRRC43, LRRC45, LRRC46, LRRC47, LRRC49, LRRC4B, LRRC4C, LRRC52, LRRC53, LRRC55, LRRC56, LRRC57, LRRC58, LRRC59, LRRC6, LRRC61, LRRC63, LRRC66, LRRC69, LRRC7, LRRC70, LRRC71, LRRC72, LRRC73, LRRC74A, LRRC74B, LRRC75A, LRRC75B, LRRC8A, LRRC8B, LRRC8C, LRRC8D, LRRC8E, LRRC9, LRRCCI, LRRD1, LRRFIP1, LRRFIP2, LRRIQ1, LRRIQ3, LRRIQ4, LRR^{k1}, LRR^{k2}, LRRN1, LRRN2, LRRN3, LRRN4, LRRN4CL, LRRTM1, LRRTM2, LRRTM3, LRRTM4, LRSAMI, LRTMI, LRTM2, LRTOMT, LRWD1, LSAMP, LSG1, LSM1, LSM10, LSM11, LSM12, LSM14A, LSM14B, LSM2, LSM3, LSM4, LSM5, LSM6, LSM7, LSM8, LSMEM1, LSMEM2, LSP1, LSR, LSS, LSTI, LTA, LTA4H, LTB, LTB4R, LTB4R2, LTBP1, LTBP2, LTBP3, LTBP4, LTBR, LTC4S, LTF, LTK, LTN1, LTV1, LUC7L, LUC7L2, LUC7L3, LUM, LURAP1, LURAPIL, LUZP1, LUZP2, LUZP4, LUZP6, LVRN, LXN, LY6D, LY6E, LY6G5B, LY6G5C, LY6G6C, LY6G6D, LY6G6E, LY6G6F, LY6H, LY6K, LY6L, LY75, LY75-CD302, LY86, LY9, LY96, LYAR, LYG1, LYG2, LYLI, LYN, LYNX1, LYPDI, LYPD2, LYPD3, LYPD4, LYPD5, LYPD6, LYPD6B, LYPD8, LYPLAI, LYPLA2, LYPLALI, LYRMI, LYRM2, LYRM4, LYRM7, LYRM9, LYSMDI, LYSMD2, LYSMD3, LYSMD4, LYST, LYVEI, LYZ, LYZLI, LYZL2, LYZL4, LYZL6, LZIC, LZTFL1, LZTRI, LZTS1, LZTS2, LZTS3, MIAP, M6PR, MAATS1, MAB21L1, MAB21L2, MAB21L3, MACC1, MACF1, MACRODI, MACROD2, MADILI, MAD2L1, MAD2LIBP, MAD2L2, MADCAMI, MADD, MAEA, MAEL, MAF, MAF1, MAFA, MAFB, MAFF, MAFG, MAFK, MAG, MAGEAI, MAGEA10, MAGEA11, MAGEA12, MAGEA2, MAGEA2B, MAGEA3, MAGEA4, MAGEA6, MAGEA8, MAGEA9, MAGEA9B, MAGEB1, MAGEB10, MAGEB16, MAGEB17, MAGEB18, MAGEB2, MAGEB3, MAGEB4, MAGEB5, MAGEB6, MAGEB6P1, MAGEC1, MAGEC2, MAGEC3, MAGED1, MAGED2, MAGED4, MAGED4B, MAGEE1, MAGEE2, MAGEF1, MAGEH1, MAGEL2, MAGII, MAGI2, MAGI3, MAGIX, MAGOH, MAGOHB, MAGTI, MAIP1, MAJIN, MAK, MAK16, MAL, MAL2, MALL, MALRDI, MALSUI, MALTI, MAMDC2, MAMDC4, MAMLI, MAML2, MAML3, MAMLDI, MAMSTR, MANIA1, MANIA2, MANIBI, MANICI, MAN2A1, MAN2A2, MAN2B1, MAN2B2, MAN2C1, MANBA, MANBAL, MANEA, MANEAL, MANF, MANSCI, MANSC4, MAOA, MAOB, MAP10, MAPIA, MAPIB, MAPILC3A, MAPILC3B, MAPILC3B2, MAPILC3C, MAP1S, MAP2, MAP2K1, MAP2K2, MAP2K3, MAP2K4, MAP2K5, MAP2K6, MAP2K7, MAP3K1, MAP3K10, MAP3K11, MAP3K12, MAP3K13, MAP3K14, MAP3K15, MAP3K19, MAP3K2, MAP3K20, MAP3K21, MAP3K3, MAP3K4, MAP3K5, MAP3K6, MAP3K7, MAP3K7CL, MAP3K8, MAP3K9, MAP4, MAP4K1, MAP4K2, MAP4K3, MAP4K4, MAP4K5, MAP6, MAP6D1, MAP7, MAP7D1, MAP7D2, MAP7D3, MAP9, MAPKI, MAPK10, MAPK11, MAPK12, MAPK13, MAPK14, MAPK15, MAPKIIPIL, MAPK3, MAPK4, MAPK6, MAPK7, MAPK8, MAPK8IP1, MAPK8IP2, MAPK8IP3, MAPK9, MAPKAPI, MAPKAPK2, MAPKAPK3, MAPKAPK5, MAPKBP1, MAPREI, MAPRE2, MAPRE3, MAPT, MARCI, MARC2, MARCHI, MARCH10, MARCH11, MARCH2, MARCH3, MARCH4, MARCH5, MARCH6, MARCH7, MARCH8, MARCH9, MARCKS, MARCKSLI, MARCO, MARFI, MAR^{k1}, MAR^{k2}, MAR^{k3}, MAR^{k4}, MARS, MARS2, MARVELDI, MARVELD2, MARVELD3, MAS1, MASIL, MASPI, MASP2, MASTI, MAST2, MAST3, MAST4, MASTL, MATIA, MAT2A, MAT2B, MATK, MATN1, MATN2, MATN3, MATN4, MATR3, MAU2, MAVS, MAX, MAZ, MB, MB21D1, MB21D2, MBDI, MBD2, MBD3, MBD3L1, MBD3L2, MBD3L2B, MBD3L3, MBD3L4, MBD3L5, MBD4, MBD5, MBD6, MBIP, MBL2, MBLAC1, MBLAC2, MBNL1, MBNL2, MBNL3, MBOATI, MBOAT2, MBOAT4, MBOAT7, MBP, MBTD1, MBTPS1, MBTPS2, MCIR, MC2R, MC3R, MC4R, MC5R, MCAM, MCAT, MCC, MCCC1, MCCC2, MCCD1, MCEE, MCEMP1, MCF2, MCF2L, MCF2L2, MCFD2, MCHR1, MCHR2, MCIDAS, MCL1, MCM10, MCM2, MCM3, MCM3AP, MCM4, MCM5, MCM6, MCM7, MCM8, MCM9, MCMBP, MCMDC2, MCOLNI, MCOLN2, MCOLN3, MCPHI, MCRIP1, MCRIP2, MCRS1, MCTP1, MCTP2, MCTS1, MCU, MCUB, MCUR1, MDC1, MDFI, MDFIC, MDFIC2, MDGA1, MDGA2, MDH1, MDHIB, MDH2, MDK, MDM1, MDM2, MDM4, MDN1, MDP1, MDS2, ME1, ME2, ME3, MEA1, MEAF6, MECOM, MECP2, MECR, MEDI, MED10, MED11, MED12, MED12L, MED13, MED13L, MED14, MED14OS, MED15, MED16, MED17, MED18, MED19, MED20, MED21, MED22, MED23, MED24, MED25, MED26, MED27, MED28, MED29, MED30, MED31, MED4, MED6, MED7, MED8, MED9, MEDAG, MEF2A, MEF2B, MEF2C, MEF2D, MEFV, MEGF10, MEGF11, MEGF6, MEGF8, MEGF9, MEI1, MEI4, MEIG1, MEIKIN, MEIOB, MEIOC, MEIS1, MEIS2, MEIS3, MELK, MELTF, MEMO1, MEN1, MEOX1, MEOX2, MEPIA, MEPIB, MEPCE, MEPE, MESD, MESP1, MESP2, MEST, MET, METAPI, METAPID, METAP2, METRN, METRNL, METTLI, METTL11B, METTL12, METTL13, METTL14, METTL15, METTL16, METTL17, METTL18, METTL21A, METTL21C, METTL22, METTL23, METTL24, METTL25, METTL26, METTL27, METTL2A, METTL2B, METTL3, METTL4, METTL5, METTL6, METTL7A, METTL7B, METTL8, METTL9, MEX3A, MEX3B, MEX3C, MEX3D, MFAP1, MFAP2, MFAP3, MFAP3L, MFAP4, MFAP5, MFF, MFGE8, MFHAS1, MFN1, MFN2, MFNG, MFRP, MFSD1, MFSD10, MFSD11, MFSD12, MFSD13A, MFSD14A, MFSD14B, MFSD14C, MFSD2A, MFSD2B, MFSD3, MFSD4A, MFSD4B, MFSD5, MFSD6, MFSD6L, MFSD7, MFSD8, MFSD9, MGA, MGAM, MGAM2, MGARP, MGATI, MGAT2, MGAT3, MGAT4A, MGAT4B, MGAT4C, MGAT4D, MGAT5, MGAT5B, MGEA5, MGLL, MGME1, MGMT, MGP, MGRNI, MGSTI, MGST2, MGST3, MIA, MIA3, MIA-RAB4B, MIB1, MIB2, MICA, MICALI, MICAL2, MICAL3, MICALCL, MICALLI, MICALL2, MICB, MICUI, MICU2, MICU3, MIDI, MIDIIP1, MID2, MIDN, MIEF1, MIEF2, MIEN1, MIERI, MIER2, MIER3, MIF, MIF4GD, MIGA1, MIGA2, MIIP, MILRI, MINDY1, MINDY2, MINDY3, MINDY4, MINDY4B, MINKI, MINOS1, MINOSI-NBLI, MINPPI, MIOS, MIOX, MIP, MIPEP, MIPOLI, MIS12, MIS18A, MIS18BP1, MISP, MISP3, MITDI, MITF, MIXL1, MKI67, MKKS, MKL1, MKL2, MKLN1, MKNK1, MKNK2, MKRN1, MKRN2, MKRN2OS, MKRN3, MKS1, MKX, MLANA, MLC1, MLEC, MLF1, MLF2, MLH1, MLH3, MLIP, MLKL, MLLT1, MLLT10, MLLT11, MLLT3, MLLT6, MLN, MLNR, MLPH, MLST8, MLX, MLXIP, MLXIPL, MLYCD, MMAA, MMAB, MMACHC, MMADHC, MMD, MMD2, MME, MMEL1, MMGT1, MMP1, MMP10, MMP11, MMP12, MMP13, MMP14, MMP15, MMP16, MMP17, MMP19, MMP2, MMP20, MMP21, MMP23B, MMP24, MMP24-AS1, MMP25, MMP26, MMP27, MMP28, MMP3, MMP7, MMP8, MMP9, MMRN1, MMRN2, MMS19, MMS22L, MN1, MNATI, MND1, MNDA, MNS1, MNT, MNX1, MOAPI, MOBIA, MOBIB, MOB2, MOB3A, MOB3B, MOB3C, MOB4, MOBP, MOCOS, MOCS1, MOCS2, MOCS3, MOG, MOGAT1, MOGAT2, MOGAT3, MOGS, MOK, MONIA, MONIB, MON2, MORCI, MORC2, MORC3, MORC4, MORF4L1, MORF4L2, MORNI, MORN2, MORN3, MORN4, MORN5, MOS, MOSPD1, MOSPD2, MOSPD3, MOV10, MOVIOLI, MOXDI, MPC1, MPCIL, MPC2, MPDU1, MPDZ, MPEG1, MPG, MPHOSPH10, MPHOSPH6, MPHOSPH8, MPHOSPH9, MPI, MPIG6B, MPL, MPLKIP, MPND, MPO, MPP1, MPP2, MPP3, MPP4, MPP5, MPP6, MPP7, MPPE1, MPPED1, MPPED2, MPRIP, MPST, MPV17, MPV17L, MPV17L2, MPZ, MPZLI, MPZL2, MPZL3, MRI, MRAP, MRAP2, MRAS, MRCI, MRC2, MREII, MREG, MRFAP1, MRFAP1L1, MRGBP, MRGPRD, MRGPRE, MRGPRF, MRGPRG, MRGPRX1, MRGPRX2, MRGPRX3, MRGPRX4, MRI1, MRLN, MRM1, MRM2, MRM3, MRNIP, MRO, MROHI, MROH2A, MROH2B, MROH5, MROH6, MROH7, MROH7-TTC4, MROH8, MROH9, MRPLI, MRPL10, MRPL11, MRPL12, MRPL13, MRPL14, MRPL15, MRPL16, MRPL17, MRPL18, MRPL19, MRPL2, MRPL20, MRPL21, MRPL22, MRPL23, MRPL24, MRPL27, MRPL28, MRPL3, MRPL30, MRPL32, MRPL33, MRPL34, MRPL35, MRPL36, MRPL37, MRPL38, MRPL39, MRPL4, MRPL40, MRPL41, MRPL42, MRPL43, MRPL44, MRPL45, MRPL46, MRPL47, MRPL48, MRPL49, MRPL50, MRPL51, MRPL52, MRPL53, MRPL54, MRPL55, MRPL57, MRPL58, MRPL9, MRPS10, MRPS11, MRPS12, MRPS14, MRPS15, MRPS16, MRPS17, MRPS18A, MRPS18B, MRPS18C, MRPS2, MRPS21, MRPS22, MRPS23, MRPS24, MRPS25, MRPS26, MRPS27, MRPS28, MRPS30, MRPS31, MRPS33, MRPS34, MRPS35, MRPS36, MRPS5, MRPS6, MRPS7, MRPS9, MRRF, MRS2, MRTO4, MRVII, MS4A1, MS4A10, MS4A12, MS4A13, MS4A14, MS4A15, MS4A2, MS4A3, MS4A4A, MS4A4E, MS4A5, MS4A6A, MS4A6E, MS4A7, MS4A8, MSANTD1, MSANTD2, MSANTD3, MSANTD3-TMEFF1, MSANTD4, MSC, MSGN1, MSH2, MSH3, MSH4, MSH5, MSH5-SAPCD1, MSH6, MSI1, MSI2, MSL1, MSL2, MSL3, MSLN, MSLNL, MSMB, MSMO1, MSMP, MSN, MSR1, MSR^{A}, MSRB1, MSRB2, MSRB3, MSS51, MST1, MSTIR, MSTN, MSTO1, MSX1, MSX2, MTIA, MTIB, MTIE, MTIF, MTIG, MT1H, MTIHL1, MTIM, MTIX, MT2A, MT3, MT4, MTA1, MTA2, MTA3, MTAP, MT-ATP6, MT-ATP8, MTBP, MTCHI, MTCH2, MTCLI, MT-CO1, MT-CO2, MT-CO3, MTCPI, MT-CYB, MTDH, MTERF1, MTERF2, MTERF3, MTERF4, MTF1, MTF2, MTFMT, MTFP1, MTFR1, MTFRIL, MTFR2, MTG1, MTG2, MTHFD1, MTHFDIL, MTHFD2, MTHFD2L, MTHFR, MTHFS, MTHFSD, MTIF2, MTIF3, MTM1, MTMR1, MTMR10, MTMR11, MTMR12, MTMR14, MTMR2, MTMR3, MTMR4, MTMR6, MTMR7, MTMR8, MTMR9, MT-ND1, MT-ND2, MT-ND3, MT-ND4, MT-ND4L, MT-ND5, MT-ND6, MTNRIA, MTNRIB, MTO1, MTOR, MTPAP, MTPN, MTR, MTRF1, MTRFIL, MTRNR2L1, MTRNR2L10, MTRNR2L11, MTRNR2L12, MTRNR2L13, MTRNR2L3, MTRNR2L4, MTRNR2L5, MTRNR2L6, MTRNR2L7, MTRNR2L8, MTRR, MTSS1, MTSSIL, MTTP, MTURN, MTUS1, MTUS2, MTX1, MTX2, MTX3, MUCI, MUC12, MUC13, MUC15, MUC16, MUC17, MUC2, MUC20, MUC21, MUC22, MUC3A, MUC4, MUC5AC, MUC5B, MUC6, MUC7, MUCLI, MULI, MUMI, MUMILI, MUS81, MUSK, MUSTN1, MUT, MUTYH, MVB12A, MVB12B, MVD, MVK, MVP, MX1, MX2, MXD1, MXD3, MXD4, MXI1, MXRA5, MXRA7, MXRA8, MYADM, MYADML2, MYB, MYBBPIA, MYBLI, MYBL2, MYBPC1, MYBPC2, MYBPC3, MYBPH, MYBPHL, MYC, MYCBP, MYCBP2, MYCBPAP, MYCL, MYCN, MYCTI, MYD88, MYDGF, MYEF2, MYEOV, MYF5, MYF6, MYHI, MYH10, MYH11, MYH13, MYH14, MYH15, MYH2, MYH3, MYH4, MYH6, MYH7, MYH7B, MYH8, MYH9, MYLI, MYL10, MYL12A, MYL12B, MYL2, MYL3, MYL4, MYL5, MYL6, MYL6B, MYL7, MYL9, MYLIP, MYLK, MYLK2, MYLK3, MYLK4, MYLPF, MYMK, MYMX, MYNN, MY010, MY015A, MYO15B, MY016, MY018A, MYO18B, MY019, MYOIA, MYO1B, MYOIC, MYOID, MYOIE, MYOIF, MYO1G, MYOIH, MY03A, MYO3B, MYO5A, MYO5B, MYO5C, MY06, MY07A, MY07B, MY09A, MYO9B, MYOC, MYOCD, MYOCOS, MYOD1, MYOF, MYOG, MYOMI, MYOM2, MYOM3, MYOT, MYOZ1, MYOZ2, MYOZ3, MYPN, MYPOP, MYRF, MYRFL, MYRIP, MYSMI, MYTI, MYTIL, MYZAP, MZB1, MZF1, MZTI, MZT2A, MZT2B, N4BP1, N4BP2, N4BP2L1, N4BP2L2, N4BP3, N6AMTI, NAA10, NAA11, NAA15, NAA16, NAA20, NAA25, NAA30, NAA35, NAA38, NAA40, NAA50, NAA60, NAAA, NAALAD2, NAALADLI, NAALADL2, NABI, NAB2, NABP1, NABP2, NACA, NACA2, NACAD, NACCI, NACC2, NADK, NADK2, NADSYNI, NAEI, NAFI, NAGA, NAGK, NAGLU, NAGPA, NAGS, NAIF1, NAIP, NALCN, NAMPT, NANOG, NANOGNB, NANOGP8, NANOS1, NANOS2, NANOS3, NANP, NANS, NAPILI, NAPIL2, NAPIL3, NAPIL4, NAPIL5, NAPA, NAPB, NAPEPLD, NAPG, NAPRT, NAPSA, NARF, NARFL, NARS, NARS2, NASP, NATI, NATI0, NAT14, NAT16, NAT2, NAT6, NAT8, NAT8B, NAT8L, NAT9, NATDI, NAVI, NAV2, NAV3, NAXD, NAXE, NBAS, NBDY, NBEA, NBEAL1, NBEAL2, NBL1, NBN, NBPF1, NBPF10, NBPF11, NBPF12, NBPF14, NBPF15, NBPF19, NBPF20, NBPF26, NBPF3, NBPF4, NBPF6, NBPF9, NBRI, NCALD, NCAMI, NCAM2, NCAN, NCAPD2, NCAPD3, NCAPG, NCAPG2, NCAPH, NCAPH2, NCBP1, NCBP2, NCBP2-AS2, NCBP2L, NCBP3, NCCRP1, NCDN, NCEHI, NCF1, NCF2, NCF4, NCKI, NCK2, NCKAP1, NCKAPIL, NCKAP5, NCKAP5L, NCKIPSD, NCL, NCLN, NCMAP, NCOAI, NCOA2, NCOA3, NCOA4, NCOA5, NCOA6, NCOA7, NCORI, NCOR2, NCR1, NCR2, NCR3, NCR3LG1, NCS1, NCSTN, NDC1, NDC80, NDE1, NDEL1, NDFIP1, NDFIP2, NDN, NDNF, NDOR1, NDP, NDRG1, NDRG2, NDRG3, NDRG4, NDST1, NDST2, NDST3, NDST4, NDUFA1, NDUFA10, NDUFA11, NDUFA12, NDUFA13, NDUFA2, NDUFA3, NDUFA4, NDUFA4L2, NDUFA5, NDUFA6, NDUFA7, NDUFA8, NDUFA9, NDUFAB1, NDUFAF1, NDUFAF2, NDUFAF3, NDUFAF4, NDUFAF5, NDUFAF6, NDUFAF7, NDUFAF8, NDUFB1, NDUFB10, NDUFB11, NDUFB2, NDUFB3, NDUFB4, NDUFB5, NDUFB6, NDUFB7, NDUFB8, NDUFB9, NDUFC1, NDUFC2, NDUFC2-KCTD14, NDUFS1, NDUFS2, NDUFS3, NDUFS4, NDUFS5, NDUFS6, NDUFS7, NDUFS8, NDUFV1, NDUFV2, NDUFV3, NEB, NEBL, NECAB1, NECAB2, NECAB3, NECAP1, NECAP2, NECTIN1, NECTIN2, NECTIN3, NECTIN4, NEDD1, NEDD4, NEDD4L, NEDD8, NEDD8-MDP1, NEDD9, NEFH, NEFL, NEFM, NEGRI, NEIL1, NEIL2, NEIL3, NEKI, NEK10, NEK11, NEK2, NEK3, NEK4, NEK5, NEK6, NEK7, NEK8, NEK9, NELFA, NELFB, NELFCD, NELFE, NELL1, NELL2, NEMF, NEMP1, NEMP2, NENF, NEO1, NEPRO, NES, NETI, NETO1, NETO2, NEU1, NEU2, NEU3, NEU4, NEURLI, NEURLIB, NEURL2, NEURL3, NEURL4, NEURODI, NEUROD2, NEUROD4, NEUROD6, NEUROG1, NEUROG2, NEUROG3, NEXMIF, NEXN, NF1, NF2, NFAM1, NFASC, NFAT5, NFATCI, NFATC2, NFATC2IP, NFATC3, NFATC4, NFE2, NFE2LI, NFE2L2, NFE2L3, NFE4, NFIA, NFIB, NFIC, NFIL3, NFIX, NFKB1, NFKB2, NFKBIA, NFKBIB, NFKBID, NFKBIE, NFKBILI, NFKBIZ, NFRKB, NFS1, NFU1, NFX1, NFXLI, NFYA, NFYB, NFYC, NGB, NGDN, NGEF, NGF, NGFR, NGLY1, NGRN, NHEJ1, NHLHI, NHLH2, NHLRCI, NHLRC2, NHLRC3, NHLRC4, NHP2, NHS, NHSL1, NHSL2, NICN1, NID1, NID2, NIF3L1, NIFK, NIMIK, NIN, NINJI, NINJ2, NINL, NIP7, NIPA1, NIPA2, NIPALI, NIPAL2, NIPAL3, NIPAL4, NIPBL, NIPSNAPI, NIPSNAP2, NIPSNAP3A, NIPSNAP3B, NISCH, NITI, NIT2, NKAINI, NKAIN2, NKAIN3, NKAIN4, NKAP, NKAPL, NKD1, NKD2, NKG7, NKIRAS1, NKIRAS2, NKPD1, NKRF, NKTR, NKX1-1, NKX1-2, NKX2-1, NKX2-2, NKX2-3, NKX2-4, NKX2-5, NKX2-6, NKX2-8, NKX3-1, NKX3-2, NKX6-1, NKX6-2, NKX6-3, NLE1, NLGN1, NLGN2, NLGN3, NLGN4X, NLGN4Y, NLK, NLN, NLRC3, NLRC4, NLRC5, NLRP1, NLRP10, NLRP11, NLRP12, NLRP13, NLRP14, NLRP2, NLRP2B, NLRP3, NLRP4, NLRP5, NLRP6, NLRP7, NLRP8, NLRP9, NLRX1, NMB, NMBR, NMD3, NME1, NME1-NME2, NME2, NME3, NME4, NME5, NME6, NME7, NME8, NME9, NMI, NMNATI, NMNAT2, NMNAT3, NMRALI, NMRKI, NMR^{k2}, NMS, NMT1, NMT2, NMU, NMURI, NMUR2, NNAT, NNMT, NNT, NOAI, NOBI, NOBOX, NOC2L, NOC3L, NOCAL, NOCT, NOD1, NOD2, NODAL, NOG, NOL10, NOL11, NOL12, NOL3, NOL4, NOL4L, NOL6, NOL7, NOL8, NOL9, NOLCI, NOMI, NOMO1, NOMO2, NOMO3, NONO, NOP10, NOP14, NOP16, NOP2, NOP53, NOP56, NOP58, NOP9, NOS1, NOSIAP, NOS2, NOS3, NOSIP, NOSTRIN, NOTCHI, NOTCH2, NOTCH2NL, NOTCH3, NOTCH4, NOTO, NOTUM, NOV, NOVAI, NOVA2, NOXI, NOX3, NOX4, NOX5, NOXAI, NOXO1, NOXREDI, NPAPI, NPASI, NPAS2, NPAS3, NPAS4, NPAT, NPB, NPBWR1, NPBWR2, NPC1, NPCILI, NPC2, NPDC1, NPEPLI, NPEPPS, NPFF, NPFFR1, NPFFR2, NPHP1, NPHP3, NPHP3-ACAD11, NPHP4, NPHS1, NPHS2, NPIPA1, NPIPA2, NPIPA3, NPIPA5, NPIPA7, NPIPA8, NPIPB11, NPIPB12, NPIPB13, NPIPB15, NPIPB2, NPIPB3, NPIPB4, NPIPB5, NPIPB6, NPIPB7, NPIPB8, NPIPB9, NPL, NPLOC4, NPMI, NPM2, NPM3, NPNT, NPPA, NPPB, NPPC, NPR1, NPR2, NPR3, NPRL2, NPRL3, NPS, NPSR1, NPTN, NPTX1, NPTX2, NPTXR, NPVF, NPW, NPY, NPYIR, NPY2R, NPY4R, NPY4R2, NPY5R, NQO1, NQ02, NROB1, NROB2, NR1D1, NR1D2, NR1H2, NR1H3, NR1H4, NR112, NR113, NR2C1, NR2C2, NR2C2AP, NR2E1, NR2E3, NR2F1, NR2F2, NR2F6, NR3C1, NR3C2, NR4A1, NR4A2, NR4A3, NR5A1, NR5A2, NR6A1, NRAP, NRARP, NRAS, NRBF2, NRBP1, NRBP2, NRCAM, NRDC, NRDE2, NREP, NRF1, NRG1, NRG2, NRG3, NRG4, NRGN, NRIP1, NRIP2, NRIP3, NRK, NRL, NRM, NRN1, NRNIL, NRP1, NRP2, NRROS, NRSN1, NRSN2, NRTN, NRXN1, NRXN2, NRXN3, NSA2, NSD1, NSD2, NSD3, NSDHL, NSF, NSFLIC, NSL1, NSMAF, NSMCE1, NSMCE2, NSMCE3, NSMCE4A, NSMF, NSRP1, NSUN2, NSUN3, NSUN4, NSUN5, NSUN6, NSUN7, NT5C, NT5CIA, NT5C1B, NT5C1B-RDH14, NT5C2, NT5C3A, NT5C3B, NT5DC1, NT5DC2, NT5DC3, NT5DC4, NT5E, NT5M, NTAN1, NTF3, NTF4, NTHL1, NTM, NTMTI, NTN1, NTN3, NTN4, NTN5, NTNG1, NTNG2, NTPCR, NTR^{k1}, NTR^{k2}, NTR^{k3}, NTS, NTSRI, NTSR2, NUAKI, NUAK2, NUBI, NUBPI, NUBP2, NUBPL, NUCB1, NUCB2, NUCKS1, NUDC, NUDCD1, NUDCD2, NUDCD3, NUDTI, NUDT10, NUDT11, NUDT12, NUDT13, NUDT14, NUDT15, NUDT16, NUDT16L1, NUDT17, NUDT18, NUDT19, NUDT2, NUDT21, NUDT22, NUDT3, NUDT4, NUDT4P1, NUDT5, NUDT6, NUDT7, NUDT8, NUDT9, NUF2, NUFIP1, NUFIP2, NUGGC, NUMA1, NUMB, NUMBL, NUP107, NUP133, NUP153, NUP155, NUP160, NUP188, NUP205, NUP210, NUP210L, NUP214, NUP35, NUP37, NUP43, NUP50, NUP54, NUP58, NUP62, NUP62CL, NUP85, NUP88, NUP93, NUP98, NUPL2, NUPRI, NUPR2, NUSI, NUSAP1, NUTF2, NUTMI, NUTM2A, NUTM2B, NUTM2D, NUTM2E, NUTM2F, NUTM2G, NVL, NWD1, NWD2, NXF1, NXF2, NXF2B, NXF3, NXF5, NXN, NXNL1, NXNL2, NXPE1, NXPE2, NXPE3, NXPE4, NXPHI, NXPH2, NXPH3, NXPH4, NXT1, NXT2, NYAP1, NYAP2, NYNRIN, NYX, OAF, OARD1, OAS1, OAS2, OAS3, OASL, OAT, OAZI, OAZ2, OAZ3, OBP2A, OBP2B, OBSCN, OBSCN-ASI, OBSLI, OC90, OCA2, OCELI, OCIADI, OCIAD2, OCLM, OCLN, OCM, OCM2, OCRL, OCSTAMP, ODAM, ODC1, ODF1, ODF2, ODF2L, ODF3, ODF3B, ODF3L1, ODF3L2, ODF4, OFCCI, OFD1, OGDH, OGDHL, OGFOD1, OGFOD2, OGFOD3, OGFR, OGFRLI, OGGI, OGN, OGT, OIP5, OIT3, OLA1, OLAH, OLFM1, OLFM2, OLFM3, OLFM4, OLFML1, OLFML2A, OLFML2B, OLFML3, OLIG1, OLIG2, OLIG3, OLRI, OMA1, OMD, OMG, OMP, ONECUTI, ONECUT2, ONECUT3, OOEP, OOSP2, OPA1, OPA3, OPALIN, OPCML, OPHNI, OPLAH, OPNILW, OPNIMW, OPNIMW2, OPNIMW3, OPNISW, OPN3, OPN4, OPN5, OPRD1, OPRKI, OPRL1, OPRMI, OPRPN, OPTC, OPTN, OR10A2, OR10A3, OR10A4, OR10A5, OR10A6, OR10A7, OR10ACI, OR10ADI, OR10AGI, OR10C1, OR10D3, OR10G2, OR10G3, OR10G4, OR10G6, OR10G7, OR10G8, OR10G9, OR10H1, OR10H2, OR10H3, OR10H4, OR10H5, OR10JI, OR10J3, OR10J4, OR10J5, OR10K1, OR10K2, OR10P1, OR10Q1, OR10R², OR10S1, OR10T2, OR10V1, OR10W1, OR10X¹, OR10Z₁, OR11A1, OR11G2, OR11H1, OR11H12, OR11H2, OR11H4, OR11H6, OR11H7, OR11L1, OR12D1, OR12D2, OR12D3, OR13A1, OR13C2, OR13C3, OR13C4, OR13C5, OR13C7, OR13C8, OR13C9, OR13D1, OR13F1, OR13G1, OR13H1, OR13J1, OR14A16, OR14A2, OR14C36, OR1411, OR14JI, OR14K1, ORIAI, ORIA2, OR1B1, ORIC1, ORID2, ORID5, ORIE1, ORIE2, ORIF1, ORIGI, ORIII, ORIJI, ORIJ2, ORIJ4, ORIK1, ORIL1, ORIL3, ORIL4, ORIL6, ORIL8, ORIM1, ORINI, ORIN2, ORIP1, ORIQ1, ORIS1, ORIS2, OR2A1, OR2A12, OR2A14, OR2A2, OR2A25, OR2A4, OR2A42, OR2A5, OR2A7, OR2AE1, OR2AG1, OR2AG2, OR2AJ1, OR2AK2, OR2AP1, OR2AT4, OR2B11, OR2B2, OR2B3, OR2B6, OR2C1, OR2C3, OR2D2, OR2D3, OR2F1, OR2F2, OR2G2, OR2G3, OR2G6, OR2H1, OR2H2, OR2J1, OR2J2, OR2J3, OR2K2, OR2L13, OR2L2, OR2L3, OR2L5, OR2L8, OR2M2, OR2M3, OR2M4, OR2M5, OR2M7, OR2S2, OR2T1, OR2T10, OR2T11, OR2T12, OR2T2, OR2T27, OR2T29, OR2T3, OR2T33, OR2T34, OR2T35, OR2T4, OR2T5, OR2T6, OR2T7, OR2T8, OR2V1, OR2V2, OR2W1, OR2W3, OR2Y₁, OR2Z₁, OR3A1, OR3A2, OR3A3, OR4A15, OR4A16, OR4A47, OR4A5, OR4A8, OR4B1, OR4C11, OR4C12, OR4C13, OR4C15, OR4C16, OR4C3, OR4C45, OR4C46, OR4C5, OR4C6, OR4D1, OR4D10, OR4D11, OR4D2, OR4D5, OR4D6, OR4D9, OR4E1, OR4E2, OR4F15, OR4F16, OR4F17, OR4F21, OR4F29, OR4F3, OR4F4, OR4F5, OR4F6, OR4K1, OR4K13, OR4K14, OR4K15, OR4K17, OR4K2, OR4K3, OR4K5, OR4L1, OR4M1, OR4M2, OR4N₂, OR4N₄, OR4N₅, OR4P4, OR4Q2, OR4Q3, OR4S1, OR4S2, OR4X¹, OR4X², OR51A2, OR51A4, OR51A7, OR51B2, OR51B4, OR51B5, OR51B6, OR51D1, OR51E1, OR51E2, OR51F1, OR51F2, OR51G1, OR51G2, OR51H1, OR5111, OR5112, OR51JI, OR5ILI, OR51MI, OR51Q1, OR51SI, OR51TI, OR51VI, OR52A1, OR52A5, OR52B2, OR52B4, OR52B6, OR52D1, OR52E2, OR52E4, OR52E5, OR52E6, OR52E8, OR52H1, OR5211, OR5212, OR52J3, OR52K1, OR52K2, OR52L1, OR52M1, OR52NI, OR52N₂, OR52N₄, OR52N₅, OR52R¹, OR52W1, OR52Z₁, OR56A1, OR56A3, OR56A4, OR56A5, OR56B1, OR56B4, OR5A1, OR5A2, OR5ACI, OR5AC2, OR5AK2, OR5ANI, OR5AP2, OR5ARI, OR5AS1, OR5AU1, OR5B12, OR5B17, OR5B2, OR5B21, OR5B3, OR5C1, OR5D13, OR5D14, OR5D16, OR5D18, OR5F1, OR5G3, OR5H1, OR5H14, OR5H15, OR5H2, OR5H6, OR5H8, OR511, OR5J2, OR5K1, OR5K2, OR5K3, OR5K4, OR5L1, OR5L2, OR5M1, OR5M10, OR5M11, OR5M3, OR5M8, OR5M9, OR5P2, OR5P3, OR5R¹, OR5T1, OR5T2, OR5T3, OR5V1, OR5W2, OR6A2, OR6B1, OR6B2, OR6B3, OR6C1, OR6C2, OR6C3, OR6C4, OR6C6, OR6C65, OR6C68, OR6C70, OR6C74, OR6C75, OR6C76, OR6F1, OR6J1, OR6K2, OR6K3, OR6K6, OR6M1, OR6N₁, OR6N₂, OR6P1, OR6Q1, OR6S1, OR6T1, OR6V1, OR6X¹, OR6Y1, OR7A10, OR7A17, OR7A5, OR7C1, OR7C2, OR7D2, OR7D4, OR7E24, OR7G1, OR7G2, OR7G3, OR8A1, OR8B12, OR8B2, OR8B3, OR8B4, OR8B8, OR8D1, OR8D2, OR8D4, OR8G1, OR8G5, OR8H1, OR8H2, OR8H3, OR812, OR8J1, OR8J2, OR8J3, OR8K1, OR8K3, OR8K5, OR8S1, OR8U1, OR8U8, OR9A2, OR9A4, OR9G1, OR9G4, OR9G9, OR9HIP, OR911, OR9K2, OR9Q1, OR9Q2, ORAII, ORAI2, ORAI3, ORAOVI, ORCI, ORC2, ORC3, ORC4, ORC5, ORC6, ORMI, ORM2, ORMDLI, ORMDL2, ORMDL3, OS9, OSBP, OSBP2, OSBPL10, OSBPL11, OSBPLIA, OSBPL2, OSBPL3, OSBPL5, OSBPL6, OSBPL7, OSBPL8, OSBPL9, OSCAR, OSCP1, OSERI, OSGEP, OSGEPLI, OSGINI, OSGIN2, OSM, OSMR, OSRI, OSR2, OST4, OSTC, OSTF1, OSTM1, OSTN, OTC, OTOA, OTOF, OTOG, OTOGL, OTOLI, OTOP1, OTOP2, OTOP3, OTOR, OTOS, OTP, OTUB1, OTUB2, OTUD1, OTUD3, OTUD4, OTUD5, OTUD6A, OTUD6B, OTUD7A, OTUD7B, OTULIN, OTX1, OTX2, OVCA2, OVCHI, OVCH2, OVGP1, OVOLI, OVOL2, OVOL3, OXAIL, OXCTI, OXCT2, OXER1, OXGR1, OXLD1, OXNAD1, OXR1, OXSM, OXSR1, OXT, OXTR, P2RX1, P2RX2, P2RX3, P2RX4, P2RX5, P2RX5-TAXIBP3, P2RX6, P2RX7, P2RY1, P2RY10, P2RY11, P2RY12, P2RY13, P2RY14, P2RY2, P2RY4, P2RY6, P2RY8, P3H1, P3H2, P3H3, P3H4, P4HA1, P4HA2, P4HA3, P4HB, P4HTM, PA2G4, PAAFI, PABPCI, PABPCIL, PABPCIL2A, PABPCIL2B, PABPC3, PABPC4, PABPC4L, PABPC5, PABPN1, PABPNIL, PACRG, PACRGL, PACS1, PACS2, PACSINI, PACSIN2, PACSIN3, PADI1, PADI2, PADI3, PADI4, PADI6, PAEP, PAF1, PAFAHIBI, PAFAHIB2, PAFAHIB3, PAFAH2, PAGI, PAGEI, PAGE2, PAGE2B, PAGE3, PAGE4, PAGE5, PAGR1, PAH, PAICS, PAIP1, PAIP2, PAIP2B, PAK1, PAKIIP1, PAK2, PAK3, PAK4, PAK5, PAK6, PALB2, PALDI, PALLD, PALM, PALM2, PALM2-AKAP2, PALM3, PALMD, PAM, PAM16, PAMR1, PAN2, PAN3, PANK1, PANK2, PANK3, PANK4, PANO1, PANX1, PANX2, PANX3, PAOX, PAPD4, PAPD5, PAPD7, PAPLN, PAPOLA, PAPOLB, PAPOLG, PAPPA, PAPPA2, PAPSS1, PAPSS2, PAQR3, PAQR4, PAQR5, PAQR6, PAQR7, PAQR8, PAQR9, PARD3, PARD3B, PARD6A, PARD6B, PARD6G, PARG, PAR^{k7}, PARL, PARMI, PARN, PARP1, PARPIO, PARP11, PARP12, PARP14, PARP15, PARP16, PARP2, PARP3, PARP4, PARP6, PARP8, PARP9, PARPBP, PARS2, PARVA, PARVB, PARVG, PASDI, PASK, PATEI, PATE2, PATE3, PATE4, PATJ, PATLI, PATL2, PATZI, PAWR, PAX1, PAX2, PAX3, PAX4, PAX5, PAX6, PAX7, PAX8, PAX9, PAXBPI, PAXIP1, PAXX, PBDC1, PBK, PBLD, PBOV1, PBRMI, PBX1, PBX2, PBX3, PBX4, PBXIP1, PC, PCBD1, PCBD2, PCBP1, PCBP2, PCBP3, PCBP4, PCCA, PCCB, PCDHI, PCDH10, PCDH11X, PCDH11Y, PCDH12, PCDH15, PCDH17, PCDH18, PCDH19, PCDH₂O, PCDH7, PCDH8, PCDH9, PCDHAI, PCDHA10, PCDHA11, PCDHA12, PCDHA13, PCDHA2, PCDHA3, PCDHA4, PCDHA5, PCDHA6, PCDHA7, PCDHA8, PCDHA9, PCDHACI, PCDHAC2, PCDHB1, PCDHB10, PCDHB11, PCDHB12, PCDHB13, PCDHB14, PCDHB15, PCDHB16, PCDHB2, PCDHB3, PCDHB4, PCDHB5, PCDHB6, PCDHB7, PCDHB8, PCDHB9, PCDHGAI, PCDHGA10, PCDHGA11, PCDHGA12, PCDHGA2, PCDHGA3, PCDHGA4, PCDHGA5, PCDHGA6, PCDHGA7, PCDHGA8, PCDHGA9, PCDHGB1, PCDHGB2, PCDHGB3, PCDHGB4, PCDHGB5, PCDHGB6, PCDHGB7, PCDHGC3, PCDHGC4, PCDHGC5, PCEDIA, PCEDIB, PCF11, PCGF1, PCGF2, PCGF3, PCGF5, PCGF6, PCID2, PCIF1, PCK1, PCK2, PCLAF, PCLO, PCM1, PCMT1, PCMTD1, PCMTD2, PCNA, PCNP, PCNT, PCNX1, PCNX2, PCNX3, PCNX4, PCOLCE, PCOLCE2, PCOTH, PCP2, PCP4, PCP4L1, PCSKI, PCSKIN, PCSK2, PCSK4, PCSK5, PCSK6, PCSK7, PCSK9, PCTP, PCYOX1, PCYOXIL, PCYTIA, PCYTIB, PCYT2, PDAP1, PDC, PDCD1, PDCD10, PDCD11, PDCDILG2, PDCD2, PDCD2L, PDCD4, PDCD5, PDCD6, PDCD6IP, PDCD7, PDCL, PDCL2, PDCL3, PDE10A, PDE11A, PDE12, PDEIA, PDEIB, PDEIC, PDE2A, PDE3A, PDE3B, PDE4A, PDE4B, PDE4C, PDE4D, PDE4DIP, PDE5A, PDE6A, PDE6B, PDE6C, PDE6D, PDE6G, PDE6H, PDE7A, PDE7B, PDE8A, PDE8B, PDE9A, PDF, PDGFA, PDGFB, PDGFC, PDGFD, PDGFR^{A}, PDGFRB, PDGFRL, PDHA1, PDHA2, PDHB, PDHX, PDIA2, PDIA3, PDIA4, PDIA5, PDIA6, PDIKIL, PDILT, PDK1, PDK2, PDK3, PDK4, PDLIMI, PDLIM2, PDLIM3, PDLIM4, PDLIM5, PDLIM7, PDP1, PDP2, PDPKI, PDPN, PDPR, PDRGI, PDS5A, PDS5B, PDSSI, PDSS2, PDX1, PDXDC1, PDXK, PDXP, PDYN, PDZD11, PDZD2, PDZD3, PDZD4, PDZD7, PDZD8, PDZD9, PDZK1, PDZKIIP1, PDZRN3, PDZRN4, PEA15, PEAK1, PEARI, PEBP1, PEBP4, PECAMI, PECR, PEF1, PEG10, PEG3, PELI1, PELI2, PELI3, PELO, PELP1, PEMT, PENK, PEPD, PER1, PER2, PER3, PERMI, PERP, PES1, PET100, PET117, PEX1, PEX10, PEX11A, PEX11B, PEX11G, PEX12, PEX13, PEX14, PEX16, PEX19, PEX2, PEX26, PEX3, PEX5, PEX5L, PEX6, PEX7, PF4, PF4V1, PFAS, PFDN1, PFDN2, PFDN4, PFDN5, PFDN6, PFKFB1, PFKFB2, PFKFB3, PFKFB4, PFKL, PFKM, PFKP, PFN1, PFN2, PFN3, PFN4, PGA3, PGA4, PGA5, PGAMI, PGAM2, PGAM4, PGAM5, PGAP1, PGAP2, PGAP3, PGBD1, PGBD2, PGBD4, PGBD5, PGC, PGD, PGF, PGGHG, PGGTIB, PGK1, PGK2, PGLS, PGLYRP1, PGLYRP2, PGLYRP3, PGLYRP4, PGM1, PGM2, PGM2L1, PGM3, PGM5, PGP, PGPEP1, PGPEPIL, PGR, PGRMC1, PGRMC2, PGS1, PHACTRI, PHACTR2, PHACTR3, PHACTR4, PHAX, PHB, PHB2, PHC1, PHC2, PHC3, PHEX, PHF1, PHF10, PHF11, PHF12, PHF13, PHF14, PHF19, PHF2, PHF20, PHF20L1, PHF21A, PHF21B, PHF23, PHF24, PHF3, PHF5A, PHF6, PHF7, PHF8, PHGDH, PHGR1, PHIP, PHKA1, PHKA2, PHKB, PHKG1, PHKG2, PHLDA1, PHLDA2, PHLDA3, PHLDB1, PHLDB2, PHLDB3, PHLPP1, PHLPP2, PHOSPHO1, PHOSPHO2, PHOX2A, PHOX2B, PHPTI, PHRF1, PHTF1, PHTF2, PHYH, PHYHD1, PHYHIP, PHYHIPL, PHYKPL, PI15, PI16, PI3, PI4K2A, PI4K2B, PI4KA, PI4 KB, PIANP, PIAS1, PIAS2, PIAS3, PIAS4, PIBF1, PICALM, PICK1, PID1, PIDD1, PIEZO1, PIEZO2, PIF1, PIFO, PIGA, PIGB, PIGBOS1, PIGC, PIGF, PIGG, PIGH, PIGK, PIGL, PIGM, PIGN, PIGO, PIGP, PIGQ, PIGR, PIGS, PIGT, PIGU, PIGV, PIGW, PIGX, PIGY, PIGZ, PIHID1, PIHID2, PIHID3, PIK3AP1, PIK3C2A, PIK3C2B, PIK3C2G, PIK3C3, PIK3CA, PIK3CB, PIK3CD, PIK3CG, PIK3IP1, PIK3R¹, PIK3R², PIK3R³, PIK3R⁴, PIK3R⁵, PIK3R⁶, PIKFYVE, PILR^{A}, PILRB, PIM1, PIM2, PIM3, PIMREG, PIN1, PIN4, PINK1, PINLYP, PINX1, PIP, PIP4K2A, PIP4K2B, PIP4K2C, PIP5KIA, PIP5KIB, PIP5KIC, PIP5KL1, PIPOX, PIR, PIRT, PISD, PITHD1, PITPNA, PITPNB, PITPNCI, PITPNMI, PITPNM2, PITPNM3, PITRMI, PITXI, PITX2, PITX3, PIWILI, PIWIL2, PIWIL3, PIWIL4, PJA1, PJA2, PKD1, PKDILI, PKD1L2, PKDIL3, PKD2, PKD2L1, PKD2L2, PKDCC, PKDREJ, PKHD1, PKHDIL1, PKIA, PKIB, PKIG, PKLR, PKM, PKMYTI, PKN1, PKN2, PKN3, PKNOX1, PKNOX2, PKP1, PKP2, PKP3, PKP4, PLAIA, PLA2G10, PLA2G12A, PLA2G12B, PLA2G15, PLA2G16, PLA2GIB, PLA2G2A, PLA2G2C, PLA2G2D, PLA2G2E, PLA2G2F, PLA2G3, PLA2G4A, PLA2G4B, PLA2G4C, PLA2G4D, PLA2G4E, PLA2G4F, PLA2G5, PLA2G6, PLA2G7, PLA2R¹, PLAA, PLACI, PLAC4, PLAC8, PLAC8LI, PLAC9, PLAGI, PLAGLI, PLAGL2, PLAT, PLAU, PLAUR, PLB1, PLBD1, PLBD2, PLCB1, PLCB2, PLCB3, PLCB4, PLCD1, PLCD3, PLCD4, PLCEI, PLCG1, PLCG2, PLCHI, PLCH2, PLCLI, PLCL2, PLCXD1, PLCXD2, PLCXD3, PLCZI, PLD1, PLD2, PLD3, PLD4, PLD5, PLD6, PLEC, PLEK, PLEK2, PLEKHAI, PLEKHA2, PLEKHA3, PLEKHA4, PLEKHA5, PLEKHA6, PLEKHA7, PLEKHA8, PLEKHB1, PLEKHB2, PLEKHD1, PLEKHF1, PLEKHF2, PLEKHG1, PLEKHG2, PLEKHG3, PLEKHG4, PLEKHG4B, PLEKHG5, PLEKHG6, PLEKHG7, PLEKHHI, PLEKHH2, PLEKHH3, PLEKHJ1, PLEKHM1, PLEKHM2, PLEKHM3, PLEKHN1, PLEKHO1, PLEKHO2, PLEKHS1, PLETI, PLG, PLGLB1, PLGLB2, PLGRKT, PLIN1, PLIN2, PLIN3, PLIN4, PLIN5, PLK1, PLK2, PLK3, PLK4, PLK5, PLLP, PLN, PLOD1, PLOD2, PLOD3, PLP1, PLP2, PLPBP, PLPP1, PLPP2, PLPP3, PLPP4, PLPP5, PLPP6, PLPP7, PLPPRI, PLPPR2, PLPPR3, PLPPR4, PLPPR5, PLRGI, PLSI, PLS3, PLSCRI, PLSCR2, PLSCR3, PLSCR4, PLSCR5, PLTP, PLVAP, PLXDCI, PLXDC2, PLXNAI, PLXNA2, PLXNA3, PLXNA4, PLXNB1, PLXNB2, PLXNB3, PLXNC1, PLXND1, PM20D1, PM20D2, PMAIP1, PMCH, PMEL, PMEPAI, PMF1, PMF1-BGLAP, PMFBP1, PML, PMM1, PMM2, PMP2, PMP22, PMPCA, PMPCB, PMS1, PMS2, PMVK, PNCK, PNISR, PNKD, PNKP, PNLDC1, PNLIP, PNLIPRP1, PNLIPRP2, PNLIPRP3, PNMA1, PNMA2, PNMA3, PNMA5, PNMA6A, PNMA6E, PNMA6F, PNMASA, PNMA8B, PNMA8C, PNMT, PNN, PNO1, PNOC, PNP, PNPLAI, PNPLA2, PNPLA3, PNPLA4, PNPLA5, PNPLA6, PNPLA7, PNPLA8, PNPO, PNPT1, PNRC1, PNRC2, POCIA, POCIB, POCIB-GALNT4, POC5, PODN, PODNL1, PODXL, PODXL2, POFIB, POFUTI, POFUT2, POGK, POGLUTI, POGZ, POLA1, POLA2, POLB, POLDI, POLD2, POLD3, POLD4, POLDIP2, POLDIP3, POLE, POLE2, POLE3, POLE4, POLG, POLG2, POLH, POLI, POLK, POLL, POLM, POLN, POLQ, POLRIA, POLRIB, POLRIC, POLRID, POLRIE, POLR2A, POLR2B, POLR2C, POLR2D, POLR2E, POLR2F, POLR2G, POLR2H, POLR2I, POLR2J, POLR2J2, POLR2J3, POLR2K, POLR2L, POLR2M, POLR3A, POLR3B, POLR3C, POLR3D, POLR3E, POLR3F, POLR3G, POLR3GL, POLR3H, POLR3K, POLRMT, POM121, POM121C, POM121L12, POM121L2, POMC, POMGNTI, POMGNT2, POMK, POMP, POMTI, POMT2, POMZP3, PONI, PON2, PON3, POP1, POP4, POP5, POP7, POPDC2, POPDC3, POR, PORCN, POSTN, POTI, POTEA, POTEB, POTEB2, POTEB3, POTEC, POTED, POTEE, POTEF, POTEG, POTEH, POTEI, POTEJ, POTEM, POUIF1, POU2AF1, POU2F1, POU2F2, POU2F3, POU3F1, POU3F2, POU3F3, POU3F4, POU4F1, POU4F2, POU4F3, POU5F1, POUSFIB, POU5F2, POU6F1, POU6F2, PP2D1, PPAI, PPA2, PPAN, PPAN-P2RY11, PPAR^{A}, PPARD, PPARG, PPARGCIA, PPARGCIB, PPAT, PPBP, PPCDC, PPCS, PPDPF, PPEF1, PPEF2, PPFIA1, PPFIA2, PPFIA3, PPFIA4, PPFIBP1, PPFIBP2, PPHLN1, PPIA, PPIAL4A, PPIAL4C, PPIAL4D, PPIAL4E, PPIAL4F, PPIAL4G, PPIB, PPIC, PPID, PPIE, PPIF, PPIG, PPIH, PPIL1, PPIL2, PPIL3, PPIL4, PPIL6, PPIP5K1, PPIP5K2, PPL, PPMIA, PPMIB, PPMID, PPMIE, PPMIF, PPMIG, PPMIH, PPMIJ, PPMIK, PPMIL, PPMIM, PPMIN, PPME1, PPOX, PPPICA, PPP1CB, PPPICC, PPPIR10, PPPIR11, PPPIR12A, PPPIR12B, PPPIR12C, PPPIR13B, PPPIR13L, PPP1R14A, PPP1R14B, PPPIR14C, PPPIR14D, PPPIR15A, PPPIR15B, PPPIR16A, PPPIR16B, PPP1R17, PPPIR18, PPPIRIA, PPPIRIB, PPPIRIC, PPPIR2, PPPIR21, PPPIR26, PPPIR27, PPP1R2P3, PPP1R2P9, PPPIR32, PPPIR35, PPPIR36, PPPIR37, PPPIR3A, PPPIR3B, PPPIR3C, PPP1R3D, PPP1R3E, PPP1R3F, PPP1R3G, PPP1R42, PPPIR7, PPPIR8, PPPIR9A, PPPIR9B, PPP2CA, PPP2CB, PPP2RIA, PPP2RIB, PPP2R2A, PPP2R2B, PPP2R2C, PPP2R2D, PPP2R3A, PPP2R3B, PPP2R3C, PPP2R5A, PPP2R5B, PPP2R5C, PPP2R5D, PPP2R5E, PPP3CA, PPP3CB, PPP3CC, PPP3R1, PPP3R2, PPP4C, PPP4R1, PPP4R2, PPP4R3A, PPP4R3B, PPP4R3CP, PPP4R4, PPP5C, PPP5D1, PPP6C, PPP6R1, PPP6R2, PPP6R3, PPRC1, PPT1, PPT2, PPT2-EGFL8, PPTC7, PPWD1, PPY, PQBP1, PQLC1, PQLC2, PQLC2L, PQLC3, PRAC1, PRAC2, PRADC1, PRAF2, PRAG1, PRAMI, PRAME, PRAMEF1, PRAMEF10, PRAMEF11, PRAMEF12, PRAMEF13, PRAMEF14, PRAMEF15, PRAMEF17, PRAMEF18, PRAMEF19, PRAMEF2, PRAMEF20, PRAMEF25, PRAMEF26, PRAMEF27, PRAMEF33, PRAMEF4, PRAMEF5, PRAMEF6, PRAMEF7, PRAMEF8, PRAMEF9, PRAP1, PRB1, PRB2, PRB3, PRB4, PRC1, PRCC, PRCD, PRCP, PRDMI, PRDM10, PRDM11, PRDM12, PRDM13, PRDM14, PRDM15, PRDM16, PRDM2, PRDM4, PRDM5, PRDM6, PRDM7, PRDM8, PRDM9, PRDX1, PRDX2, PRDX3, PRDX4, PRDX5, PRDX6, PREB, PRELIDI, PRELID2, PRELID3A, PRELID3B, PRELP, PREP, PREPL, PREX1, PREX2, PRFI, PRG2, PRG3, PRG4, PRHI, PRH2, PRICKLE1, PRICKLE2, PRICKLE3, PRICKLE4, PRIMI, PRIM2, PRIMA1, PRIMPOL, PRKAAI, PRKAA2, PRKABI, PRKAB2, PRKACA, PRKACB, PRKACG, PRKAG1, PRKAG2, PRKAG3, PRKARIA, PRKARIB, PRKAR2A, PRKAR2B, PRKCA, PRKCB, PRKCD, PRKCE, PRKCG, PRKCH, PRKCI, PRKCQ, PRKCSH, PRKCZ, PRKD1, PRKD2, PRKD3, PRKDC, PRKG1, PRKG2, PRKN, PRKR^{A}, PRKRIP1, PRKX, PRL, PRLH, PRLHR, PRLR, PRMI, PRM2, PRM3, PRMT1, PRMT2, PRMT3, PRMT5, PRMT6, PRMT7, PRMT8, PRMT9, PRND, PRNP, PRNT, PROBI, PROC, PROCAI, PROCR, PRODH, PRODH2, PROK1, PROK2, PROKRI, PROKR2, PROMI, PROM2, PROP1, PRORY, PROS1, PROSER1, PROSER2, PROSER3, PROXI, PROX2, PROZ, PRPF18, PRPF19, PRPF3, PRPF31, PRPF38A, PRPF38B, PRPF39, PRPF4, PRPF40A, PRPF40B, PRPF4B, PRPF6, PRPF8, PRPH, PRPH2, PRPS1, PRPSIL1, PRPS2, PRPSAP1, PRPSAP2, PRR11, PRR12, PRR13, PRR14, PRR14L, PRR15, PRR15L, PRR16, PRR18, PRR19, PRR20A, PRR20B, PRR20C, PRR20D, PRR20E, PRR21, PRR22, PRR23A, PRR23B, PRR23C, PRR23D1, PRR23D2, PRR25, PRR26, PRR27, PRR29, PRR3, PRR30, PRR32, PRR34, PRR35, PRR36, PRR4, PRR5, PRR5-ARHGAP8, PRR5L, PRR7, PRR9, PRRCI, PRRC2A, PRRC2B, PRRC2C, PRRG1, PRRG2, PRRG3, PRRG4, PRR^{T1}, PRR^{T2}, PRR^{T3}, PRR^{T4}, PRRX1, PRRX2, PRSS1, PRSS12, PRSS16, PRSS2, PRSS21, PRSS22, PRSS23, PRSS27, PRSS3, PRSS33, PRSS35, PRSS36, PRSS37, PRSS38, PRSS41, PRSS42, PRSS45, PRSS46, PRSS48, PRSS50, PRSS51, PRSS53, PRSS54, PRSS55, PRSS56, PRSS57, PRSS58, PRSS8, PRTFDC1, PRTG, PRTN3, PRUNE1, PRUNE2, PRX, PRY, PRY2, PSAP, PSAPLI, PSATI, PSCA, PSD, PSD2, PSD3, PSD4, PSEN1, PSEN2, PSENEN, PSG1, PSG11, PSG2, PSG3, PSG4, PSG5, PSG6, PSG7, PSG8, PSG9, PSIP1, PSKHI, PSKH2, PSMA1, PSMA2, PSMA3, PSMA4, PSMA5, PSMA6, PSMA7, PSMA8, PSMB1, PSMB10, PSMB11, PSMB2, PSMB3, PSMB4, PSMB5, PSMB6, PSMB7, PSMB8, PSMB9, PSMC1, PSMC2, PSMC3, PSMC3IP, PSMC4, PSMC5, PSMC6, PSMD1, PSMD10, PSMD11, PSMD12, PSMD13, PSMD14, PSMD2, PSMD3, PSMD4, PSMD5, PSMD6, PSMD7, PSMD8, PSMD9, PSME1, PSME2, PSME3, PSME4, PSMF1, PSMG1, PSMG2, PSMG3, PSMG4, PSORSIC1, PSORSIC2, PSPC1, PSPH, PSPN, PSRC1, PSTK, PSTPIP1, PSTPIP2, PTAFR, PTARI, PTBP1, PTBP2, PTBP3, PTCD1, PTCD2, PTCD3, PTCHI, PTCH2, PTCHD1, PTCHD3, PTCHD4, PTCR^{A}, PTDSS1, PTDSS2, PTEN, PTER, PTFIA, PTGDR, PTGDR2, PTGDS, PTGERI, PTGER2, PTGER3, PTGER4, PTGES, PTGES2, PTGES3, PTGES3L, PTGES3L-AARSDI, PTGFR, PTGFRN, PTGIR, PTGIS, PTGRI, PTGR2, PTGS1, PTGS2, PTH, PTHIR, PTH2, PTH2R, PTHLH, PTK2, PTK2B, PTK6, PTK7, PTMA, PTMS, PTN, PTOV1, PTP4A1, PTP4A2, PTP4A3, PTPA, PTPDC1, PTPMT1, PTPN1, PTPN11, PTPN12, PTPN13, PTPN14, PTPN18, PTPN2, PTPN20, PTPN21, PTPN22, PTPN23, PTPN3, PTPN4, PTPN5, PTPN6, PTPN7, PTPN9, PTPR^{A}, PTPRB, PTPRC, PTPRCAP, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRZ1, PTRH1, PTRH2, PTRHD1, PTS, PTTGI, PTTGIIP, PTTG2, PTX3, PTX4, PUDP, PUF60, PUM1, PUM2, PUM3, PURA, PURB, PURG, PUS1, PUS10, PUS3, PUS7, PUS7L, PUSLI, PVALB, PVR, PVRIG, PWP1, PWP2, PWWP2A, PWWP2B, PXDC1, PXDN, PXDNL, PXK, PXMP2, PXMP4, PXN, PXT1, PXYLP1, PYCARD, PYCRI, PYCR2, PYCR3, PYDC1, PYDC2, PYGB, PYGL, PYGM, PYGO1, PYGO2, PYHIN1, PYMI, PYROXDI, PYROXD2, PYURF, PYY, PZP, QARS, QDPR, QKI, QPCT, QPCTL, QPRT, QRFP, QRFPR, QRICHI, QRICH2, QRSLI, QSERI, QSOX1, QSOX2, QTR^{T1}, QTR^{T2}, R3HCC1, R3HCCIL, R3HDMI, R3HDM2, R3HDM4, R3HDML, RAB10, RABIIA, RABIIB, RAB11FIP1, RAB11FIP2, RAB11FIP3, RAB11FIP4, RAB11FIP5, RAB12, RAB13, RAB14, RAB15, RAB17, RAB18, RAB19, RABIA, RABIB, RAB20, RAB21, RAB22A, RAB23, RAB24, RAB25, RAB26, RAB27A, RAB27B, RAB28, RAB29, RAB2A, RAB2B, RAB30, RAB31, RAB32, RAB33A, RAB33B, RAB34, RAB35, RAB36, RAB37, RAB38, RAB39A, RAB39B, RAB3A, RAB3B, RAB3C, RAB3D, RAB3GAP1, RAB3GAP2, RAB3ILI, RAB3IP, RAB40A, RAB40AL, RAB40B, RAB40C, RAB41, RAB42, RAB43, RAB44, RAB4A, RAB4B, RAB4B-EGLN2, RAB5A, RAB5B, RAB5C, RAB6A, RAB6B, RAB6C, RAB7A, RAB7B, RAB8A, RAB8B, RAB9A, RAB9B, RABACI, RABEP1, RABEP2, RABEPK, RABGAP1, RABGAPIL, RABGEF1, RABGGTA, RABGGTB, RABIF, RABL2A, RABL2B, RABL3, RABL6, RACI, RAC2, RAC3, RACGAPI, RACKI, RADI, RAD17, RAD18, RAD21, RAD21L1, RAD23A, RAD23B, RAD50, RAD51, RAD51AP1, RAD51AP2, RAD51B, RAD51C, RAD51D, RAD52, RAD54B, RAD54L, RAD54L2, RAD9A, RAD9B, RADIL, RAEI, RAETIE, RAETIG, RAETIL, RAFI, RAGI, RAG2, RAII, RAI14, RAI2, RALA, RALB, RALBP1, RALGAPA1, RALGAPA2, RALGAPB, RALGDS, RALGPS1, RALGPS2, RALY, RALYL, RAMP1, RAMP2, RAMP3, RAN, RANBP1, RANBP10, RANBP17, RANBP2, RANBP3, RANBP3L, RANBP6, RANBP9, RANGAPI, RANGRF, RAPIA, RAPIB, RAPIGAP, RAPIGAP2, RAPIGDS1, RAP2A, RAP2B, RAP2C, RAPGEF1, RAPGEF2, RAPGEF3, RAPGEF4, RAPGEF5, RAPGEF6, RAPGEFL1, RAPHI, RAPSN, RAR^{A}, RARB, RARG, RARRESI, RARRES2, RARRES3, RARS, RARS2, RASA1, RASA2, RASA3, RASA4, RASA4B, RASALI, RASAL2, RASAL3, RASDI, RASD2, RASEF, RASGEFIA, RASGEFIB, RASGEFIC, RASGRF1, RASGRF2, RASGRP1, RASGRP2, RASGRP3, RASGRP4, RASIPI, RASLIOA, RASLIOB, RASLIIA, RASL11B, RASL12, RASSF1, RASSF10, RASSF2, RASSF3, RASSF4, RASSF5, RASSF6, RASSF7, RASSF8, RASSF9, RAVERI, RAVER2, RAX, RAX2, RBI, RBICCI, RBAK, RBAK-RBAKDN, RBBP4, RBBP5, RBBP6, RBBP7, RBBP8, RBBP8NL, RBBP9, RBCKI, RBFA, RBFOX1, RBFOX2, RBFOX3, RBKS, RBL1, RBL2, RBM10, RBM11, RBM12, RBM12B, RBM14, RBM14-RBM4, RBM15, RBM15B, RBM17, RBM18, RBM19, RBM20, RBM22, RBM23, RBM24, RBM25, RBM26, RBM27, RBM28, RBM3, RBM33, RBM34, RBM38, RBM39, RBM4, RBM41, RBM42, RBM43, RBM44, RBM45, RBM46, RBM47, RBM48, RBM4B, RBM5, RBM6, RBM7, RBM8A, RBMS1, RBMS2, RBMS3, RBMX, RBMX2, RBMXL1, RBMXL2, RBMXL3, RBMYIAI, RBMYIB, RBMYID, RBMYIE, RBMYIF, RBMYIJ, RBP1, RBP2, RBP3, RBP4, RBP5, RBP7, RBPJ, RBPJL, RBPMS, RBPMS2, RBSN, RBX1, RC3H1, RC3H2, RCAN1, RCAN2, RCAN3, RCBTB1, RCBTB2, RCC1, RCCIL, RCC2, RCCDI, RCE1, RCHY1, RCL1, RCN1, RCN2, RCN3, RCORI, RCOR2, RCOR3, RCSDI, RCVRN, RD3, RD3L, RDH10, RDH11, RDH12, RDH13, RDH14, RDH16, RDH5, RDH8, RDMI, RDX, REC114, REC8, RECK, RECQL, RECQL4, RECQL5, REEP1, REEP2, REEP3, REEP4, REEP5, REEP6, REGIA, REGIB, REG3A, REG3G, REG4, REL, RELA, RELB, RELLI, RELL2, RELN, RELT, REMI, REM2, REN, RENBP, REP15, REPINI, REPS1, REPS2, RER1, RERE, RERG, RERGL, RESP18, REST, RET, RETN, RETNLB, RETREG1, RETREG2, RETREG3, RETSAT, REVI, REV3L, REXO1, REXO2, REXO4, REX05, RFC1, RFC2, RFC3, RFC4, RFC5, RFESD, RFFL, RFK, RFLNA, RFLNB, RFNG, RFPL1, RFPL2, RFPL3, RFPL3S, RFPL4A, RFPL4AL1, RFPL4B, RFT1, RFTN1, RFTN2, RFWD2, RFWD3, RFX1, RFX2, RFX3, RFX4, RFX5, RFX6, RFX7, RFX8, RFXANK, RFXAP, RGCC, RGLI, RGL2, RGL3, RGL4, RGMA, RGMB, RGN, RGP1, RGPDI, RGPD2, RGPD3, RGPD4, RGPD5, RGPD6, RGPD8, RGR, RGSI, RGS10, RGS11, RGS12, RGS13, RGS14, RGS16, RGS17, RGS18, RGS19, RGS2, RGS20, RGS21, RGS22, RGS3, RGS4, RGS5, RGS6, RGS7, RGS7BP, RGS8, RGS9, RGS9BP, RGSL1, RHAG, RHBDD1, RHBDD2, RHBDD3, RHBDF1, RHBDF2, RHBDL1, RHBDL2, RHBDL3, RHBG, RHCE, RHCG, RHD, RHEB, RHEBL1, RHNO1, RHO, RHOA, RHOB, RHOBTB1, RHOBTB2, RHOBTB3, RHOC, RHOD, RHOF, RHOG, RHOH, RHOJ, RHOQ, RHOTI, RHOT2, RHOU, RHOV, RHOXF1, RHOXF2, RHOXF2B, RHPN1, RHPN2, RIBCI, RIBC2, RICI, RIC3, RIC8A, RIC8B, RICTOR, RIDA, RIFI, RIIAD1, RILP, RILPLI, RILPL2, RIMBP2, RIMBP3, RIMBP3B, RIMBP3C, RIMKLA, RIMKLB, RIMS1, RIMS2, RIMS3, RIMS4, RIN1, RIN2, RIN3, RING1, RINL, RINTI, RIOKI, RIOK2, RIOK3, RIOX1, RIOX2, RIPKI, RIPK2, RIPK3, RIPK4, RIPORI, RIPOR2, RIPOR3, RIPPLY1, RIPPLY2, RIPPLY3, RIT1, RIT2, RITAI, RLBP1, RLF, RLIM, RLN1, RLN2, RLN3, RMDNI, RMDN2, RMDN3, RMI1, RMI2, RMND1, RMND5A, RMND5B, RNASEI, RNASE10, RNASE11, RNASE12, RNASE13, RNASE2, RNASE3, RNASE4, RNASE6, RNASE7, RNASE8, RNASE9, RNASEHI, RNASEH2A, RNASEH2B, RNASEH2C, RNASEK, RNASEK-C17orf49, RNASEL, RNASET2, RND1, RND2, RND3, RNF10, RNF103, RNF103-CHMP3, RNF11, RNF111, RNF112, RNF113A, RNF113B, RNF114, RNF115, RNF121, RNF122, RNF123, RNF125, RNF126, RNF128, RNF13, RNF130, RNF133, RNF135, RNF138, RNF139, RNF14, RNF141, RNF144A, RNF144B, RNF145, RNF146, RNF148, RNF149, RNF150, RNF151, RNF152, RNF157, RNF165, RNF166, RNF167, RNF168, RNF169, RNF17, RNF170, RNF175, RNF180, RNF181, RNF182, RNF183, RNF185, RNF186, RNF187, RNF19A, RNF19B, RNF2, RNF20, RNF207, RNF208, RNF212, RNF212B, RNF213, RNF214, RNF215, RNF216, RNF217, RNF219, RNF220, RNF222, RNF223, RNF224, RNF225, RNF24, RNF25, RNF26, RNF31, RNF32, RNF34, RNF38, RNF39, RNF4, RNF40, RNF41, RNF43, RNF44, RNF5, RNF6, RNF7, RNF8, RNFT1, RNFT2, RNGTT, RNHI, RNLS, RNMT, RNPC3, RNPEP, RNPEPLI, RNPSI, ROBO1, ROBO2, ROBO3, ROBO4, ROCK1, ROCK2, ROGDI, ROMI, ROMO1, ROPN1, ROPNIB, ROPNIL, RORI, ROR2, ROR^{A}, RORB, RORC, ROS1, RP1, RPILI, RP2, RP9, RPA1, RPA2, RPA3, RPA4, RPAIN, RPAP1, RPAP2, RPAP3, RPE, RPE65, RPEL1, RPF1, RPF2, RPGR, RPGRIP1, RPGRIPIL, RPH3A, RPH3AL, RPIA, RPL10, RPL10A, RPLIOL, RPL11, RPL12, RPL13, RPL13A, RPL14, RPL15, RPL17, RPL17-C18orf32, RPL18, RPL18A, RPL19, RPL21, RPL22, RPL22L1, RPL23, RPL23A, RPL24, RPL26, RPL26L1, RPL27, RPL27A, RPL28, RPL29, RPL3, RPL30, RPL31, RPL32, RPL34, RPL35, RPL35A, RPL36, RPL36A, RPL36A-HNRNPH2, RPL36AL, RPL37, RPL37A, RPL38, RPL39, RPL39L, RPL3L, RPL4, RPL41, RPL5, RPL6, RPL7, RPL7A, RPL7L1, RPL8, RPL9, RPLPO, RPLP1, RPLP2, RPN1, RPN2, RPP14, RPP21, RPP25, RPP25L, RPP30, RPP38, RPP40, RPRDIA, RPRDIB, RPRD2, RPRM, RPRML, RPS10, RPS10-NUDT3, RPS11, RPS12, RPS13, RPS14, RPS15, RPS15A, RPS16, RPS17, RPS18, RPS19, RPS19BP1, RPS2, RPS20, RPS21, RPS23, RPS24, RPS25, RPS26, RPS27, RPS27A, RPS27L, RPS28, RPS29, RPS3, RPS3A, RPS4X, RPS4Y1, RPS4Y2, RPS5, RPS6, RPS6KA1, RPS6KA2, RPS6KA3, RPS6KA4, RPS6KA5, RPS6KA6, RPS6KB1, RPS6KB2, RPS6KC1, RPS6KL1, RPS7, RPS8, RPS9, RPSA, RPTN, RPTOR, RPUSD1, RPUSD2, RPUSD3, RPUSD4, RRAD, RRAGA, RRAGB, RRAGC, RRAGD, RRAS, RRAS2, RRBP1, RREB1, RRH, RRMI, RRM2, RRM2B, RRN3, RRNAD1, RRP1, RRP12, RRP15, RRPIB, RRP36, RRP7A, RRP8, RRP9, RRS1, RS1, RSADI, RSAD2, RSBN1, RSBNIL, RSCIA1, RSF1, RSG1, RSLIDI, RSL24D1, RSPHI, RSPHIOB, RSPH10B2, RSPH14, RSPH3, RSPH4A, RSPH6A, RSPH9, RSPO1, RSPO2, RSPO3, RSPO4, RSPRY1, RSRCI, RSRC2, RSRP1, RSU1, RTBDN, RTCA, RTCB, RTEL1, RTEL1-TNFRSF6B, RTF1, RTFDCI, RTKN, RTKN2, RTL1, RTL10, RTL3, RTL4, RTL5, RTL6, RTL8A, RTL8B, RTL8C, RTL9, RTN1, RTN2, RTN3, RTN4, RTN4IP1, RTN4R, RTN4RL1, RTN4RL2, RTP1, RTP2, RTP3, RTP4, RTP5, RTTN, RUBCN, RUBCNL, RUFY1, RUFY2, RUFY3, RUFY4, RUNDCI, RUNDC3A, RUNDC3B, RUNX1, RUNXITI, RUNX2, RUNX3, RUSCI, RUSC2, RUVBLI, RUVBL2, RWDD1, RWDD2A, RWDD2B, RWDD3, RWDD4, RXFP1, RXFP2, RXFP3, RXFP4, RXR^{A}, RXRB, RXRG, RYBP, RYK, RYRI, RYR2, RYR3, S100A1, S100A10, S100A11, S100A12, S100A13, S100A14, S100A16, S100A2, S100A3, S100A4, S100A5, S100A6, S100A7, S100A7A, S100A7L2, S100A8, S100A9, S100B, S100G, S100P, S100PBP, S100Z, SIPRI, SIPR2, SIPR3, SIPR4, SIPR5, SAA1, SAA2, SAA2-SAA4, SAA4, SAALI, SAC3D1, SACMIL, SACS, SAEI, SAFB, SAFB2, SAG, SAGEI, SALLI, SALL2, SALL3, SALL4, SAMDI, SAMD10, SAMD11, SAMD12, SAMD13, SAMD14, SAMD15, SAMD3, SAMD4A, SAMD4B, SAMD5, SAMD7, SAMD8, SAMD9, SAMD9L, SAMHD1, SAMM50, SAMSNI, SAP130, SAP18, SAP25, SAP30, SAP30BP, SAP30L, SAPCD1, SAPCD2, SARIA, SARIB, SARAF, SARDH, SARMI, SARNP, SARS, SARS2, SARTI, SAR^{T3}, SASHI, SASH3, SASS6, SATI, SAT2, SATB1, SATB2, SATLI, SAVI, SAXO1, SAXO2, SAYSDI, SBDS, SBF1, SBF2, SBK1, SBK2, SBK3, SBNO1, SBNO2, SBSN, SBSPON, SC5D, SCAFI, SCAF11, SCAF4, SCAF8, SCAI, SCAMPI, SCAMP2, SCAMP3, SCAMP4, SCAMP5, SCAND1, SCAP, SCAPER, SCARA3, SCARA5, SCARB1, SCARB2, SCARF1, SCARF2, SCARTI, SCCPDH, SCD, SCD5, SCEL, SCFD1, SCFD2, SCG2, SCG3, SCG5, SCGB1A1, SCGBIC1, SCGBIC2, SCGB1D1, SCGBID2, SCGBID4, SCGB2A1, SCGB2A2, SCGB2B2, SCGB3A1, SCGB3A2, SCGN, SCHIP1, SCIMP, SCIN, SCLTI, SCLY, SCMHI, SCML1, SCML2, SCML4, SCN10A, SCN11A, SCNIA, SCNIB, SCN2A, SCN2B, SCN3A, SCN3B, SCN4A, SCN4B, SCN5A, SCN7A, SCN8A, SCN9A, SCNMI, SCNNIA, SCNNIB, SCNNID, SCNNIG, SCO1, SCO2, SCOC, SCP2, SCP2D1, SCPEP1, SCRG1, SCRIB, SCRNI, SCRN2, SCRN3, SCRTI, SCR^{T2}, SCT, SCTR, SCUBE1, SCUBE2, SCUBE3, SCX, SCYL1, SCYL2, SCYL3, SDADI, SDC1, SDC2, SDC3, SDC4, SDCBP, SDCBP2, SDCCAG3, SDCCAG8, SDE2, SDF2, SDF2L1, SDF4, SDHA, SDHAF1, SDHAF2, SDHAF3, SDHAF4, SDHB, SDHC, SDHD, SDK1, SDK2, SDR16C5, SDR39U1, SDR42E1, SDR42E2, SDR9C7, SDS, SDSL, SEBOX, SEC11A, SEC11C, SEC13, SEC14L1, SEC14L2, SEC14L3, SEC14L4, SEC14L5, SEC14L6, SEC16A, SEC16B, SEC22A, SEC22B, SEC22C, SEC23A, SEC23B, SEC23IP, SEC24A, SEC24B, SEC24C, SEC24D, SEC31A, SEC31B, SEC61A1, SEC61A2, SEC61B, SEC61G, SEC62, SEC63, SECISBP2, SECISBP2L, SECTMI, SEHIL, SELIL, SELIL2, SELIL3, SELE, SELENBP1, SELENOF, SELENOH, SELENOI, SELENOK, SELENOM, SELENON, SELENOO, SELENOP, SELENOS, SELENOT, SELENOV, SELENOW, SELL, SELP, SELPLG, SEMI, SEMA3A, SEMA3B, SEMA3C, SEMA3D, SEMA3E, SEMA3F, SEMA3G, SEMA4A, SEMA4B, SEMA4C, SEMA4D, SEMA4F, SEMA4G, SEMASA, SEMA5B, SEMA6A, SEMA6B, SEMA6C, SEMA6D, SEMA7A, SEMG1, SEMG2, SENP1, SENP2, SENP3, SENP3-EIF4A1, SENP5, SENP6, SENP7, SENP8, SEPHS1, SEPHS2, SEPSECS, SEPT1, SEPT10, SEPT11, SEPT12, SEPT14, SEPT2, SEPT3, SEPT4, SEPT5, SEPT6, SEPT7, SEPT8, SEPT9, SERACI, SERBP1, SERFIA, SERFIB, SERF2, SERGEF, SERHL2, SERINCI, SERINC2, SERINC3, SERINC4, SERINC5, SERPI, SERP2, SERPINA1, SERPINA10, SERPINA11, SERPINA12, SERPINA2, SERPINA3, SERPINA4, SERPINA5, SERPINA6, SERPINA7, SERPINA9, SERPINB1, SERPINB10, SERPINB11, SERPINB12, SERPINB13, SERPINB2, SERPINB3, SERPINB4, SERPINB5, SERPINB6, SERPINB7, SERPINB8, SERPINB9, SERPINCI, SERPIND1, SERPINE1, SERPINE2, SERPINE3, SERPINF1, SERPINF2, SERPING1, SERPINHI, SERPINI1, SERPINI2, SERTADI, SERTAD2, SERTAD3, SERTAD4, SERTMI, SESNI, SESN2, SESN3, SESTDI, SET, SETBPI, SETDIA, SETDIB, SETD2, SETD3, SETD4, SETD5, SETD6, SETD7, SETD9, SETDB1, SETDB2, SETMAR, SETSIP, SETX, SEZ6, SEZ6L, SEZ6L2, SF1, SF3A1, SF3A2, SF3A3, SF3B1, SF3B2, SF3B3, SF3B4, SF3B5, SF3B6, SFI1, SFMBT1, SFMBT2, SFN, SFPQ, SFR1, SFRP1, SFRP2, SFRP4, SFRP5, SFSWAP, SFT2D1, SFT2D2, SFT2D3, SFTA2, SFTA3, SFTPA1, SFTPA2, SFTPB, SFTPC, SFTPD, SFXN1, SFXN2, SFXN3, SFXN4, SFXN5, SGCA, SGCB, SGCD, SGCE, SGCG, SGCZ, SGF29, SGIP1, SGK1, SGK2, SGK3, SGK494, SGMS1, SGMS2, SGO1, SGO2, SGPLI, SGPP1, SGPP2, SGSH, SGSMI, SGSM2, SGSM3, SGTA, SGTB, SH2B1, SH2B2, SH2B3, SH2DIA, SH2D1B, SH2D2A, SH2D3A, SH2D3C, SH2D4A, SH2D4B, SH2D5, SH2D6, SH2D7, SH3BGR, SH3BGRL, SH3BGRL2, SH3BGRL3, SH3BP1, SH3BP2, SH3BP4, SH3BP5, SH3BP5L, SH3D19, SH3D21, SH3GL1, SH3GL2, SH3GL3, SH3GLB1, SH3GLB2, SH3KBP1, SH3PXD2A, SH3PXD2B, SH3RF1, SH3RF2, SH3RF3, SH3TC1, SH3TC2, SH3YL1, SHANK1, SHANK2, SHANK3, SHARPIN, SHB, SHBG, SHC1, SHC2, SHC3, SHC4, SHCBP1, SHCBPIL, SHD, SHE, SHF, SHH, SHISA2, SHISA3, SHISA4, SHISA5, SHISA6, SHISA7, SHISA8, SHISA9, SHKBP1, SHMTI, SHMT2, SHOC2, SHOX, SHOX2, SHPK, SHPRH, SHQ1, SHROOMI, SHROOM2, SHROOM3, SHROOM4, SHTN1, SI, SIAE, SIAHI, SIAH2, SIAH3, SIDT1, SIDT2, SIGIRR, SIGLEC1, SIGLEC10, SIGLEC11, SIGLEC12, SIGLEC14, SIGLEC15, SIGLEC5, SIGLEC6, SIGLEC7, SIGLEC8, SIGLEC9, SIGLECL1, SIGMAR1, SIKI, SIK2, SIK3, SIKE1, SILI, SIMI, SIM2, SIMC1, SIN3A, SIN3B, SIPAI, SIPAILI, SIPAIL2, SIPAIL3, SIRPA, SIRPB1, SIRPB2, SIRPD, SIRPG, SIR^{T1}, SIR^{T2}, SIR^{T3}, SIR^{T4}, SIR^{T5}, SIR^{T6}, SIR^{T7}, SITI, SIVAI, SIXI, SIX2, SIX3, SIX4, SIX5, SIX6, SKAI, SKA2, SKA3, SKAP1, SKAP2, SKI, SKIDA1, SKIL, SKIV2L, SKIV2L2, SKOR1, SKOR2, SKP1, SKP2, SLA, SLA2, SLAINI, SLAIN2, SLAMF1, SLAMF6, SLAMF7, SLAMF8, SLAMF9, SLBP, SLC10A1, SLC10A2, SLC10A3, SLC10A4, SLC10A5, SLC10A6, SLC10A7, SLC11A1, SLC11A2, SLC12A1, SLC12A2, SLC12A3, SLC12A4, SLC12A5, SLC12A6, SLC12A7, SLC12A8, SLC12A9, SLC13A1, SLC13A2, SLC13A3, SLC13A4, SLC13A5, SLC14A1, SLC14A2, SLC15A1, SLC15A2, SLC15A3, SLC15A4, SLC15A5, SLC16A1, SLC16A10, SLC16A11, SLC16A12, SLC16A13, SLC16A14, SLC16A2, SLC16A3, SLC16A4, SLC16A5, SLC16A6, SLC16A7, SLC16A8, SLC16A9, SLC17A1, SLC17A2, SLC17A3, SLC17A4, SLC17A5, SLC17A6, SLC17A7, SLC17A8, SLC17A9, SLC18A1, SLC18A2, SLC18A3, SLC18B1, SLC19A1, SLC19A2, SLC19A3, SLC1A1, SLC1A2, SLCIA3, SLCIA4, SLC1A5, SLCIA6, SLCIA7, SLC20A1, SLC20A2, SLC22A1, SLC22A10, SLC22A11, SLC22A12, SLC22A13, SLC22A14, SLC22A15, SLC22A16, SLC22A17, SLC22A18, SLC22A18AS, SLC22A2, SLC22A23, SLC22A24, SLC22A25, SLC22A3, SLC22A31, SLC22A4, SLC22A5, SLC22A6, SLC22A7, SLC22A8, SLC22A9, SLC23A1, SLC23A2, SLC23A3, SLC24A1, SLC24A2, SLC24A3, SLC24A4, SLC24A5, SLC25A1, SLC25A10, SLC25A11, SLC25A12, SLC25A13, SLC25A14, SLC25A15, SLC25A16, SLC25A17, SLC25A18, SLC25A19, SLC25A2, SLC25A20, SLC25A21, SLC25A22, SLC25A23, SLC25A24, SLC25A25, SLC25A26, SLC25A27, SLC25A28, SLC25A29, SLC25A3, SLC25A30, SLC25A31, SLC25A32, SLC25A33, SLC25A34, SLC25A35, SLC25A36, SLC25A37, SLC25A38, SLC25A39, SLC25A4, SLC25A40, SLC25A41, SLC25A42, SLC25A43, SLC25A44, SLC25A45, SLC25A46, SLC25A47, SLC25A48, SLC25A5, SLC25A51, SLC25A52, SLC25A53, SLC25A6, SLC26A1, SLC26A10, SLC26A11, SLC26A2, SLC26A3, SLC26A4, SLC26A5, SLC26A6, SLC26A7, SLC26A8, SLC26A9, SLC27A1, SLC27A2, SLC27A3, SLC27A4, SLC27A5, SLC27A6, SLC28A1, SLC28A2, SLC28A3, SLC29A1, SLC29A2, SLC29A3, SLC29A4, SLC2A1, SLC2A10, SLC2A11, SLC2A12, SLC2A13, SLC2A14, SLC2A2, SLC2A3, SLC2A4, SLC2A4RG, SLC2A5, SLC2A6, SLC2A7, SLC2A8, SLC2A9, SLC30A1, SLC30A10, SLC30A2, SLC30A3, SLC30A4, SLC30A5, SLC30A6, SLC30A7, SLC30A8, SLC30A9, SLC31A1, SLC31A2, SLC32A1, SLC33A1, SLC34A1, SLC34A2, SLC34A3, SLC35A1, SLC35A2, SLC35A3, SLC35A4, SLC35A5, SLC35B1, SLC35B2, SLC35B3, SLC35B4, SLC35C1, SLC35C2, SLC35D1, SLC35D2, SLC35D3, SLC35E1, SLC35E2, SLC35E2B, SLC35E3, SLC35E4, SLC35F1, SLC35F2, SLC35F3, SLC35F4, SLC35F5, SLC35F6, SLC35G1, SLC35G2, SLC35G3, SLC35G4, SLC35G5, SLC35G6, SLC36A1, SLC36A2, SLC36A3, SLC36A4, SLC37A1, SLC37A2, SLC37A3, SLC37A4, SLC38A1, SLC38A10, SLC38A11, SLC38A2, SLC38A3, SLC38A4, SLC38A5, SLC38A6, SLC38A7, SLC38A8, SLC38A9, SLC39A1, SLC39A10, SLC39A11, SLC39A12, SLC39A13, SLC39A14, SLC39A2, SLC39A3, SLC39A4, SLC39A5, SLC39A6, SLC39A7, SLC39A8, SLC39A9, SLC3A1, SLC3A2, SLC40A1, SLC41A1, SLC41A2, SLC41A3, SLC43A1, SLC43A2, SLC43A3, SLC44A1, SLC44A2, SLC44A3, SLC44A4, SLC44A5, SLC45A1, SLC45A2, SLC45A3, SLC45A4, SLC46A1, SLC46A2, SLC46A3, SLC47A1, SLC47A2, SLC48A1, SLC4A1, SLC4A10, SLC4A11, SLC4A1AP, SLC4A2, SLC4A3, SLC4A4, SLC4A5, SLC4A7, SLC4A8, SLC4A9, SLC50A1, SLC51A, SLC51B, SLC52A1, SLC52A2, SLC52A3, SLC5A1, SLC5A10, SLC5A11, SLC5A12, SLC5A2, SLC5A3, SLC5A4, SLC5A5, SLC5A6, SLC5A7, SLC5A8, SLC5A9, SLC6A1, SLC6A11, SLC6A12, SLC6A13, SLC6A14, SLC6A15, SLC6A16, SLC6A17, SLC6A18, SLC6A19, SLC6A2, SLC6A20, SLC6A3, SLC6A4, SLC6A5, SLC6A6, SLC6A7, SLC6A8, SLC6A9, SLC7A1, SLC7A10, SLC7A11, SLC7A13, SLC7A14, SLC7A2, SLC7A3, SLC7A4, SLC7A5, SLC7A6, SLC7A6OS, SLC7A7, SLC7A8, SLC7A9, SLC8A1, SLC8A2, SLC8A3, SLC8B1, SLC9A1, SLC9A2, SLC9A3, SLC9A3R1, SLC9A3R2, SLC9A4, SLC9A5, SLC9A6, SLC9A7, SLC9A8, SLC9A9, SLC9B1, SLC9B2, SLC9C1, SLC9C2, SLCO1A2, SLCO1B1, SLCO1B3, SLCO1B7, SLCO1C1, SLCO2A1, SLCO2B1, SLCO3A1, SLCO4A1, SLCO4C1, SLCO5A1, SLCO6A1, SLF1, SLF2, SLFN11, SLFN12, SLFN12L, SLFN13, SLFN14, SLFN5, SLFNL1, SLIRP, SLIT1, SLIT2, SLIT3, SLITRKI, SLITR^{k2}, SLITR^{k3}, SLITR^{k4}, SLITR^{k5}, SLITR^{k6}, SLK, SLMAP, SLN, SLPI, SLTM, SLU7, SLURP1, SLURP2, SLXIA, SLXIB, SLX4, SLX4IP, SMAD1, SMAD2, SMAD3, SMAD4, SMAD5, SMAD6, SMAD7, SMAD9, SMAGP, SMAPI, SMAP2, SMARCB1, SMARCCI, SMARCC2, SMARCD1, SMARCD2, SMARCD3, SMARCEI, SMCIA, SMCIB, SMC2, SMC3, SMC4, SMC5, SMC6, SMCHDI, SMCO1, SMCO2, SMC03, SMC04, SMCP, SMCR8, SMDTI, SMG1, SMG5, SMG6, SMG7, SMG8, SMG9, SMIMI, SMIM10, SMIM10L1, SMIM10L2A, SMIM10L2B, SMIM11A, SMIM11B, SMIM12, SMIM13, SMIM14, SMIM15, SMIM17, SMIM18, SMIM19, SMIM2, SMIM20, SMIM21, SMIM22, SMIM23, SMIM24, SMIM26, SMIM27, SMIM28, SMIM29, SMIM3, SMIM30, SMIM31, SMIM4, SMIM5, SMIM6, SMIM7, SMIM8, SMIM9, SMKR1, SMLRI, SMN1, SMN2, SMNDC1, SMO, SMOC1, SMOC2, SMOX, SMPDI, SMPD2, SMPD3, SMPD4, SMPDL3A, SMPDL3B, SMPX, SMR3A, SMR3B, SMS, SMTN, SMTNL1, SMTNL2, SMUI, SMUG1, SMURF1, SMURF2, SMYDI, SMYD2, SMYD3, SMYD4, SMYD5, SNAII, SNAI2, SNAI3, SNAP23, SNAP25, SNAP29, SNAP47, SNAP91, SNAPCI, SNAPC2, SNAPC3, SNAPC4, SNAPC5, SNAPIN, SNCA, SNCAIP, SNCB, SNCG, SND1, SNED1, SNF8, SNHG28, SNIP1, SNN, SNPH, SNRK, SNRNP200, SNRNP25, SNRNP27, SNRNP35, SNRNP40, SNRNP48, SNRNP70, SNRPA, SNRPAI, SNRPB, SNRPB2, SNRPC, SNRPD1, SNRPD2, SNRPD3, SNRPE, SNRPF, SNRPG, SNRPN, SNTAI, SNTB1, SNTB2, SNTG1, SNTG2, SNTN, SNU13, SNUPN, SNURF, SNW1, SNX1, SNX10, SNX11, SNX12, SNX13, SNX14, SNX15, SNX16, SNX17, SNX18, SNX19, SNX2, SNX20, SNX21, SNX22, SNX24, SNX25, SNX27, SNX29, SNX3, SNX30, SNX31, SNX32, SNX33, SNX4, SNX5, SNX6, SNX7, SNX8, SNX9, SOATI, SOAT2, SOBP, SOCSI, SOCS2, SOCS3, SOCS4, SOCS5, SOCS6, SOCS7, SOD1, SOD2, SOD3, SOGA1, SOGA3, SOHLHI, SOHLH2, SON, SORBS1, SORBS2, SORBS3, SORCS1, SORCS2, SORCS3, SORD, SORLI, SORTI, SOS1, SOS2, SOST, SOSTDCI, SOWAHA, SOWAHB, SOWAHC, SOWAHD, SOXI, SOX10, SOX11, SOX12, SOX13, SOX14, SOX15, SOX17, SOX18, SOX2, SOX21, SOX3, SOX30, SOX4, SOX5, SOX6, SOX7, SOX8, SOX9, SP1, SP100, SP110, SP140, SP140L, SP2, SP3, SP4, SP5, SP6, SP7, SP8, SP9, SPA17, SPAAR, SPACAI, SPACA3, SPACA4, SPACA5, SPACA5B, SPACA6, SPACA7, SPACA9, SPAGI, SPAG11A, SPAG11B, SPAG16, SPAG17, SPAG4, SPAG5, SPAG6, SPAG7, SPAG8, SPAG9, SPAM1, SPANXA1, SPANXA2, SPANXB1, SPANXC, SPANXD, SPANXNI, SPANXN2, SPANXN3, SPANXN4, SPANXN5, SPARC, SPARCLI, SPART, SPAST, SPATAI, SPATA12, SPATA13, SPATA16, SPATA17, SPATA18, SPATA19, SPATA2, SPATA20, SPATA21, SPATA22, SPATA24, SPATA25, SPATA2L, SPATA3, SPATA31A1, SPATA31A3, SPATA31A5, SPATA31A6, SPATA31A7, SPATA31D1, SPATA31D3, SPATA31D4, SPATA31E1, SPATA32, SPATA33, SPATA4, SPATA45, SPATA46, SPATA5, SPATASLI, SPATA6, SPATA6L, SPATA7, SPATA8, SPATA9, SPATCI, SPATCIL, SPATS1, SPATS2, SPATS2L, SPC24, SPC25, SPCS1, SPCS2, SPCS3, SPDEF, SPDLI, SPDYA, SPDYC, SPDYE1, SPDYE16, SPDYE2, SPDYE2B, SPDYE3, SPDYE4, SPDYE5, SPDYE6, SPECC1, SPECCIL, SPECCIL-ADORA2A, SPEF1, SPEF2, SPEG, SPEMI, SPEN, SPERT, SPESP1, SPG11, SPG21, SPG7, SPHAR, SPHK1, SPHK2, SPHKAP, SPII, SPIB, SPIC, SPICE1, SPIDR, SPIN1, SPIN2A, SPIN2B, SPIN3, SPIN4, SPINK1, SPINK13, SPINK14, SPINK2, SPINK4, SPINK5, SPINK6, SPINK7, SPINK8, SPINK9, SPINTI, SPINT2, SPINT3, SPINT4, SPIREI, SPIRE2, SPN, SPNSI, SPNS2, SPNS3, SPO11, SPOCD1, SPOCK1, SPOCK2, SPOCK3, SPONI, SPON2, SPOP, SPOPL, SPOUT1, SPP1, SPP2, SPPL2A, SPPL2B, SPPL2C, SPPL3, SPR, SPRED1, SPRED2, SPRED3, SPRN, SPRRIA, SPRRIB, SPRR2A, SPRR2B, SPRR2D, SPRR2E, SPRR2F, SPRR2G, SPRR3, SPRR4, SPRR5, SPRTN, SPRY1, SPRY2, SPRY3, SPRY4, SPRYD3, SPRYD4, SPRYD7, SPSB1, SPSB2, SPSB3, SPSB4, SPTA1, SPTANI, SPTB, SPTBN1, SPTBN2, SPTBN4, SPTBN5, SPTLC1, SPTLC2, SPTLC3, SPTSSA, SPTSSB, SPTY2D1, SPTY2DI-ASI, SPX, SPZI, SQLE, SQOR, SQSTMI, SRAI, SRBDI, SRC, SRCAP, SRCIN1, SRD5A1, SRD5A2, SRD5A3, SREBF1, SREBF2, SREK1, SREKIIP1, SRF, SRFBP1, SRGAP1, SRGAP2, SRGAP2B, SRGAP2C, SRGAP3, SRGN, SRI, SRL, SRM, SRMS, SRP14, SRP19, SRP54, SRP68, SRP72, SRP9, SRPK1, SRPK2, SRPK3, SRPR^{A}, SRPRB, SRPX, SRPX2, SRR, SRRD, SRRM1, SRRM2, SRRM3, SRRM4, SRRM5, SRRT, SRSF1, SRSF10, SRSF11, SRSF12, SRSF2, SRSF3, SRSF4, SRSF5, SRSF6, SRSF7, SRSF8, SRSF9, SRXNI, SRY, SS18, SS18L1, SS18L2, SSB, SSBP1, SSBP2, SSBP3, SSBP4, SSC4D, SSC5D, SSFA2, SSH1, SSH2, SSH3, SSMEMI, SSNA1, SSPN, SSPO, SSR1, SSR2, SSR3, SSR4, SSRP1, SSSCA1, SST, SSTR1, SSTR2, SSTR3, SSTR4, SSTR5, SSU72, SSU72P8, SSUH2, SSX1, SSX2, SSX2B, SSX2IP, SSX3, SSX4, SSX4B, SSX5, SSX7, ST13, ST14, ST18, ST20, ST20-MTHFS, ST3GAL1, ST3GAL2, ST3GAL3, ST3GAL4, ST3GAL5, ST3GAL6, ST5, ST6GALI, ST6GAL2, ST6GALNACI, ST6GALNAC2, ST6GALNAC3, ST6GALNAC4, ST6GALNAC5, ST6GALNAC6, ST7, ST7L, ST8SIA1, ST8SIA2, ST8SIA3, ST8SIA4, ST8SIA5, ST8SIA6, STABI, STAB2, STAC, STAC2, STAC3, STAGI, STAG2, STAG3, STAM, STAM2, STAMBP, STAMBPLI, STAPI, STAP2, STAR, STARD10, STARD13, STARD3, STARD3NL, STARD4, STARD5, STARD6, STARD7, STARD8, STARD9, STAT3, STAU1, STAU2, STBDI, STC1, STC2, STEAP1, STEAPIB, STEAP2, STEAP3, STEAP4, STH, STIL, STIM1, STIM2, STIP1, STK10, STK11, STK11IP, STK16, STK17A, STK17B, STK19, STK24, STK25, STK26, STK3, STK31, STK32A, STK32B, STK32C, STK33, STK35, STK36, STK38, STK38L, STK39, STK4, STK40, STKLD1, STMN1, STMN2, STMN3, STMN4, STMND1, STN1, STOM, STOML1, STOML2, STOML3, STON1, STON1-GTF2AIL, STON2, STOX1, STOX2, STPG1, STPG2, STPG3, STPG4, STRA6, STRA8, STRADA, STRADB, STRAP, STRBP, STRC, STRIP1, STRIP2, STRN, STRN3, STRN4, STS, STT3A, STT3B, STUB1, STUM, STX10, STX11, STX12, STX16, STX16-NPEPLI, STX17, STX18, STX19, STXIA, STX1B, STX2, STX3, STX4, STX5, STX6, STX7, STX8, STXBP1, STXBP2, STXBP3, STXBP4, STXBP5, STXBP5L, STXBP6, STYK1, STYX, STYXLI, SUB1, SUCLA2, SUCLGI, SUCLG2, SUCNRI, SUCO, SUDS3, SUFU, SUGCT, SUGP1, SUGP2, SUGTI, SULF1, SULF2, SULTIA1, SULTIA2, SULTIA3, SULTIA4, SULTIB1, SULTIC2, SULTIC3, SULTIC4, SULTIEI, SULT2A1, SULT2B1, SULT4A1, SULT6B1, SUMF1, SUMF2, SUMO1, SUMO2, SUMO3, SUMO4, SUN1, SUN2, SUN3, SUN5, SUOX, SUPT16H, SUPT20H, SUPT3H, SUPT4H1, SUPT5H, SUPT6H, SUPT7L, SUPV3L1, SURFI, SURF2, SURF4, SURF6, SUSDI, SUSD2, SUSD3, SUSD4, SUSD5, SUSD6, SUV39H1, SUV39H2, SUZ12, SV2A, SV2B, SV2C, SVBP, SVEP1, SVIL, SVIP, SVOP, SVOPL, SWAP70, SWI5, SWSAP1, SWT1, SYAPI, SYBU, SYCE1, SYCEIL, SYCE2, SYCE3, SYCN, SYCP1, SYCP2, SYCP2L, SYCP3, SYDE1, SYDE2, SYF2, SYK, SYMPK, SYN1, SYN2, SYN3, SYNC, SYNCRIP, SYNDIG1, SYNDIGIL, SYNE1, SYNE2, SYNE3, SYNE4, SYNGAP1, SYNGRI, SYNGR2, SYNGR3, SYNGR4, SYNJI, SYNJ2, SYNJ2BP, SYNJ2BP-COX16, SYNM, SYNPO, SYNPO2, SYNPO2L, SYNPR, SYNRG, SYP, SYPLI, SYPL2, SYSI, SYS1-DBNDD2, SYTI, SYT10, SYT11, SYT12, SYT13, SYT14, SYT15, SYT16, SYT17, SYT2, SYT3, SYT4, SYT5, SYT6, SYT7, SYT8, SYT9, SYTLI, SYTL2, SYTL3, SYTL4, SYTL5, SYVNI, SZRD1, SZT2, T, TAARI, TAAR2, TAAR5, TAAR6, TAAR8, TAAR9, TABI, TAB2, TAB3, TACI, TAC3, TAC4, TACCI, TACC2, TACC3, TACO1, TACRI, TACR2, TACR3, TACSTD2, TADA1, TADA2A, TADA2B, TADA3, TAF1, TAF10, TAF11, TAF12, TAF13, TAF15, TAFIA, TAFIB, TAFIC, TAFID, TAFIL, TAF2, TAF3, TAF4, TAF4B, TAF5, TAF5L, TAF6, TAF6L, TAF7, TAF7L, TAF8, TAF9, TAF9B, TAGAP, TAGLN, TAGLN2, TAGLN3, TALI, TAL2, TALDO1, TAMM41, TANCI, TANC2, TANGO2, TANGO6, TANK, TAOK1, TAOK2, TAOK3, TAPI, TAP2, TAPBP, TAPBPL, TAPTI, TARBP1, TARBP2, TARDBP, TARMI, TARS, TARS2, TARSL2, TASIR1, TASIR2, TASIR3, TAS2RI, TAS2R10, TAS2R13, TAS2R14, TAS2R16, TAS2R19, TAS2R20, TAS2R3, TAS2R30, TAS2R31, TAS2R38, TAS2R39, TAS2R4, TAS2R40, TAS2R41, TAS2R42, TAS2R43, TAS2R46, TAS2R5, TAS2R50, TAS2R60, TAS2R7, TAS2R8, TAS2R9, TASPI, TAT, TATDNI, TATDN2, TATDN3, TAXIBPI, TAXIBP3, TAZ, TBATA, TBCID1, TBCID10A, TBCID10B, TBC1D10C, TBCID12, TBCID13, TBC1D14, TBCID15, TBC1D16, TBC1D17, TBC1D19, TBC1D2, TBC1D20, TBC1D21, TBCID22A, TBC1D22B, TBC1D23, TBC1D24, TBC1D25, TBC1D26, TBC1D28, TBCID29, TBC1D2B, TBC1D3, TBC1D30, TBC1D31, TBC1D32, TBC1D3B, TBC1D3C, TBC1D3D, TBC1D3E, TBC1D3F, TBC1D3G, TBC1D3H, TBC1D3I, TBC1D3K, TBC1D3L, TBC1D4, TBC1D5, TBC1D7, TBC1D8, TBCID8B, TBC1D9, TBC1D9B, TBCA, TBCB, TBCC, TBCCD1, TBCD, TBCE, TBCEL, TBCK, TBK1, TBKBP1, TBLIX, TBLIXRI, TBLIY, TBL2, TBL3, TBP, TBPL1, TBPL2, TBR1, TBRG1, TBRG4, TBX1, TBX10, TBX15, TBX18, TBX19, TBX2, TBX20, TBX21, TBX22, TBX3, TBX4, TBX5, TBX6, TBXA2R, TBXASI, TC2N, TCAF1, TCAF2, TCAIM, TCAP, TCEA1, TCEA2, TCEA3, TCEALI, TCEAL2, TCEAL3, TCEAL4, TCEAL5, TCEAL6, TCEAL7, TCEAL8, TCEAL9, TCEANC, TCEANC2, TCERG1, TCERGIL, TCF12, TCF15, TCF19, TCF20, TCF21, TCF23, TCF24, TCF25, TCF3, TCF4, TCF7, TCF7L1, TCF7L2, TCFL5, TCHH, TCHHL1, TCHP, TCIRGI, TCLIA, TCLIB, TCN1, TCN2, TCOF1, TCP1, TCP10, TCP10L, TCP10L2, TCP11, TCP11L1, TCP11L2, TCP11X², TCTA, TCTE1, TCTE3, TCTEX1D1, TCTEXID2, TCTEXID4, TCTN1, TCTN2, TCTN3, TDG, TDGF1, TDO2, TDP1, TDP2, TDRD1, TDRD10, TDRD12, TDRD15, TDRD3, TDRD5, TDRD6, TDRD7, TDRD9, TDRKH, TDRP, TEAD1, TEAD2, TEAD3, TEAD4, TEC, TECPRI, TECPR2, TECR, TECRL, TECTA, TECTB, TEDDMI, TEF, TEFM, TEK, TEKTI, TEKT2, TEKT3, TEKT4, TEKT5, TELO2, TEN1, TENI-CDK3, TENMI, TENM2, TENM3, TENM4, TEP1, TEPP, TEPSIN, TERBI, TERB2, TERFI, TERF2, TERF2IP, TERT, TES, TESC, TESKI, TESK2, TESMIN, TESPA1, TET1, TET2, TET3, TEX10, TEX101, TEX11, TEX12, TEX13A, TEX13B, TEX13C, TEX13D, TEX14, TEX15, TEX19, TEX2, TEX22, TEX26, TEX261, TEX264, TEX28, TEX29, TEX30, TEX33, TEX35, TEX36, TEX37, TEX38, TEX43, TEX44, TEX45, TEX46, TEX47, TEX48, TEX49, TEX50, TEX51, TEX9, TF, TFAM, TFAP2A, TFAP2B, TFAP2C, TFAP2D, TFAP2E, TFAP4, TFBIM, TFB2M, TFCP2, TFCP2L1, TFDP1, TFDP2, TFDP3, TFE3, TFEB, TFEC, TFF1, TFF2, TFF3, TFG, TFIP11, TFPI, TFPI2, TFPT, TFR2, TFRC, TG, TGDS, TGFA, TGFB1, TGFBIII, TGFB2, TGFB3, TGFBI, TGFBR1, TGFBR2, TGFBR3, TGFBR3L, TGFBRAP1, TGIF1, TGIF2, TGIF2-C20orf24, TGIF2LX, TGIF2LY, TGM1, TGM2, TGM3, TGM4, TGM5, TGM6, TGM7, TGOLN2, TGS1, TH, THADA, THAP1, THAP10, THAP11, THAP12, THAP2, THAP3, THAP4, THAP5, THAP6, THAP7, THAP8, THAP9, THBD, THBS1, THBS2, THBS3, THBS4, THEG, THEGL, THEM4, THEM5, THEM6, THEMIS, THEMIS2, THGIL, THNSL1, THNSL2, THOCI, THOC2, THOC3, THOC5, THOC6, THOC7, THOP1, THPO, THR^{A}, THRAP3, THRB, THRSP, THSD1, THSD4, THSD7A, THSD7B, THTPA, THUMPD1, THUMPD2, THUMPD3, THY1, THYN1, TIA1, TIAF1, TIALI, TIAMI, TIAM2, TICAMI, TICAM2, TICRR, TIEI, TIFA, TIFAB, TIGAR, TIGD1, TIGD2, TIGD3, TIGD4, TIGD5, TIGD6, TIGD7, TIGIT, TIMD4, TIMELESS, TIMM10, TIMM10B, TIMM13, TIMM17A, TIMM17B, TIMM21, TIMM22, TIMM23, TIMM23B, TIMM29, TIMM44, TIMM50, TIMM8A, TIMM8B, TIMM9, TIMMDC1, TIMP1, TIMP2, TIMP3, TIMP4, TINAG, TINAGLI, TINCR, TINF2, TIPARP, TIPIN, TIPRL, TIRAP, TISP43, TJAP1, TJP1, TJP2, TJP3, TKI, TK2, TKFC, TKT, TKTL1, TKTL2, TLCDI, TLCD2, TLDC1, TLDC2, TLE1, TLE2, TLE3, TLE4, TLE6, TLK1, TLK2, TLLI, TLL2, TLN1, TLN2, TLNRD1, TLR1, TLR10, TLR2, TLR3, TLR4, TLR5, TLR6, TLR7, TLR8, TLR9, TLX1, TLX2, TLX3, TM2D1, TM2D2, TM2D3, TM4SF1, TM4SF18, TM4SF19, TM4SF19-TCTEX1D2, TM4SF20, TM4SF4, TM4SF5, TM6SF1, TM6SF2, TM7SF2, TM7SF3, TM9SF1, TM9SF2, TM9SF3, TM9SF4, TMA16, TMA7, TMBIM1, TMBIM4, TMBIM6, TMC1, TMC2, TMC3, TMC4, TMC5, TMC6, TMC7, TMC8, TMCC1, TMCC2, TMCC3, TMC01, TMC02, TMCO3, TMC04, TMCOSA, TMC06, TMED1, TMED10, TMED2, TMED3, TMED4, TMED5, TMED6, TMED7, TMED7-TICAM2, TMED8, TMED9, TMEFF1, TMEFF2, TMEM100, TMEM101, TMEM102, TMEM104, TMEM105, TMEM106A, TMEM106B, TMEM106C, TMEM107, TMEM108, TMEM109, TMEM11, TMEM110, TMEM110-MUSTN1, TMEM114, TMEM115, TMEM116, TMEM117, TMEM119, TMEM120A, TMEM120B, TMEM121, TMEM121B, TMEM123, TMEM125, TMEM126A, TMEM126B, TMEM127, TMEM128, TMEM129, TMEM130, TMEM131, TMEM131L, TMEM132A, TMEM132B, TMEM132C, TMEM132D, TMEM132E, TMEM133, TMEM134, TMEM135, TMEM136, TMEM138, TMEM139, TMEM140, TMEM141, TMEM143, TMEM144, TMEM145, TMEM147, TMEM14A, TMEM14B, TMEM14C, TMEM150A, TMEM150B, TMEM150C, TMEM151A, TMEM151B, TMEM154, TMEM155, TMEM156, TMEM158, TMEM159, TMEM160, TMEM161A, TMEM161B, TMEM163, TMEM164, TMEM165, TMEM167A, TMEM167B, TMEM168, TMEM169, TMEM17, TMEM170A, TMEM170B, TMEM171, TMEM173, TMEM174, TMEM175, TMEM176A, TMEM176B, TMEM177, TMEM178A, TMEM178B, TMEM179, TMEM179B, TMEM18, TMEM181, TMEM182, TMEM183A, TMEM184A, TMEM184B, TMEM184C, TMEM185A, TMEM185B, TMEM186, TMEM187, TMEM189, TMEM189-UBE2V1, TMEM19, TMEM190, TMEM191B, TMEM191C, TMEM192, TMEM196, TMEM198, TMEM199, TMEM2, TMEM200A, TMEM200B, TMEM200C, TMEM201, TMEM202, TMEM203, TMEM204, TMEM205, TMEM206, TMEM207, TMEM208, TMEM209, TMEM210, TMEM211, TMEM212, TMEM213, TMEM214, TMEM215, TMEM216, TMEM217, TMEM218, TMEM219, TMEM220, TMEM221, TMEM222, TMEM223, TMEM225, TMEM225B, TMEM229A, TMEM229B, TMEM230, TMEM231, TMEM232, TMEM233, TMEM234, TMEM235, TMEM236, TMEM237, TMEM238, TMEM239, TMEM240, TMEM241, TMEM242, TMEM243, TMEM244, TMEM245, TMEM246, TMEM247, TMEM248, TMEM249, TMEM25, TMEM250, TMEM251, TMEM252, TMEM253, TMEM254, TMEM255A, TMEM255B, TMEM256, TMEM256-PLSCR3, TMEM257, TMEM258, TMEM259, TMEM26, TMEM260, TMEM262, TMEM263, TMEM265, TMEM266, TMEM267, TMEM268, TMEM269, TMEM27, TMEM270, TMEM30A, TMEM30B, TMEM31, TMEM33, TMEM35A, TMEM35B, TMEM37, TMEM38A, TMEM38B, TMEM39A, TMEM39B, TMEM40, TMEM41A, TMEM41B, TMEM42, TMEM43, TMEM44, TMEM45A, TMEM45B, TMEM47, TMEM5, TMEM50A, TMEM50B, TMEM51, TMEM52, TMEM52B, TMEM53, TMEM54, TMEM55A, TMEM55B, TMEM56, TMEM56-RWDD3, TMEM57, TMEM59, TMEM59L, TMEM60, TMEM61, TMEM62, TMEM63A, TMEM63B, TMEM63C, TMEM64, TMEM65, TMEM67, TMEM68, TMEM69, TMEM70, TMEM71, TMEM72, TMEM74, TMEM74B, TMEM78, TMEM79, TMEM80, TMEM81, TMEM82, TMEM86A, TMEM86B, TMEM87A, TMEM87B, TMEM88, TMEM88B, TMEM89, TMEM8A, TMEM8B, TMEM9, TMEM91, TMEM92, TMEM94, TMEM95, TMEM97, TMEM98, TMEM99, TMEM9B, TMF1, TMIE, TMIGD1, TMIGD2, TMIGD3, TMLHE, TMOD1, TMOD2, TMOD3, TMOD4, TMPO, TMPPE, TMPRSS11A, TMPRSS11B, TMPRSS11D, TMPRSS11E, TMPRSS11F, TMPRSS12, TMPRSS13, TMPRSS15, TMPRSS2, TMPRSS3, TMPRSS4, TMPRSS4-AS1, TMPRSS5, TMPRSS6, TMPRSS7, TMPRSS9, TMSB10, TMSB15A, TMSB15B, TMSB4X, TMSB4Y, TMTC1, TMTC2, TMTC3, TMTC4, TMUB1, TMUB2, TMX1, TMX2, TMX2-CTNND1, TMX3, TMX4, TNC, TNF, TNFAIP1, TNFAIP2, TNFAIP3, TNFAIP6, TNFAIP8, TNFAIP8L1, TNFAIP8L2, TNFAIP8L3, TNFRSF10A, TNFRSF10B, TNFRSF10C, TNFRSF10D, TNFRSF11A, TNFRSF11B, TNFRSF12A, TNFRSF13B, TNFRSF13C, TNFRSF14, TNFRSF17, TNFRSF18, TNFRSF19, TNFRSFIA, TNFRSFIB, TNFRSF21, TNFRSF25, TNFRSF4, TNFRSF6B, TNFRSF8, TNFRSF9, TNFSF10, TNFSF11, TNFSF12, TNFSF12-TNFSF13, TNFSF13, TNFSF13B, TNFSF14, TNFSF15, TNFSF18, TNFSF4, TNFSF8, TNFSF9, TNIK, TNIP1, TNIP2, TNIP3, TNK1, TNK2, TNKS, TNKS1BP1, TNKS2, TNMD, TNN, TNNC1, TNNC2, TNNII, TNNI2, TNNI3, TNNI3K, TNNT1, TNNT2, TNNT3, TNP1, TNP2, TNPO1, TNPO2, TNPO3, TNR, TNRC18, TNRC6A, TNRC6B, TNRC6C, TNS1, TNS2, TNS3, TNS4, TNXB, TOBI, TOB2, TOEI, TOGARAMI, TOGARAM2, TOLLIP, TOMI, TOMILI, TOMIL2, TOMM20, TOMM20L, TOMM22, TOMM34, TOMM40, TOMM40L, TOMM5, TOMM6, TOMM7, TOMM70, TONSL, TOP1, TOPIMT, TOP2A, TOP2B, TOP3A, TOP3B, TOPAZI, TOPBP1, TOPORS, TORIA, TORIAIP1, TORIAIP2, TORIB, TOR2A, TOR3A, TOR4A, TOX, TOX2, TOX3, TOX4, TP53, TP53AIP1, TP53BP1, TP53BP2, TP53I11, TP53113, TP5313, TP53INP1, TP53INP2, TP53RK, TP53TG3, TP53TG3B, TP53TG3C, TP53TG3D, TP53TG3E, TP53TG3F, TP53TG5, TP63, TP73, TPBG, TPBGL, TPCN1, TPCN2, TPD52, TPD52L1, TPD52L2, TPD52L3, TPGS1, TPGS2, TPHI, TPH2, TPII, TPKI, TPM1, TPM2, TPM3, TPM4, TPMT, TPO, TPP1, TPP2, TPPP, TPPP2, TPPP3, TPR, TPRA1, TPRG1, TPRGIL, TPRKB, TPRN, TPRX1, TPSAB1, TPSB2, TPSD1, TPSG1, TPST1, TPST2, TPTI, TPTE, TPTE2, TPX2, TRA2A, TRA2B, TRABD, TRABD2A, TRABD2B, TRAC, TRADD, TRAFI, TRAF2, TRAF3, TRAF3IP1, TRAF3IP2, TRAF3IP3, TRAF4, TRAF5, TRAF6, TRAF7, TRAFDI, TRAIP, TRAJI, TRAJ10, TRAJ11, TRAJ12, TRAJ13, TRAJ14, TRAJ16, TRAJ17, TRAJ18, TRAJ19, TRAJ2, TRAJ20, TRAJ21, TRAJ22, TRAJ23, TRAJ24, TRAJ25, TRAJ26, TRAJ27, TRAJ28, TRAJ29, TRAJ3, TRAJ30, TRAJ31, TRAJ32, TRAJ33, TRAJ34, TRAJ35, TRAJ36, TRAJ37, TRAJ38, TRAJ39, TRAJ4, TRAJ40, TRAJ41, TRAJ42, TRAJ43, TRAJ44, TRAJ45, TRAJ46, TRAJ47, TRAJ48, TRAJ49, TRAJ5, TRAJ50, TRAJ52, TRAJ53, TRAJ54, TRAJ56, TRAJ57, TRAJ58, TRAJ59, TRAJ6, TRAJ61, TRAJ7, TRAJ9, TRAKI, TRAK2, TRAMI, TRAMIL1, TRAM2, TRANK1, TRAP1, TRAPPCI, TRAPPC10, TRAPPC11, TRAPPC12, TRAPPC13, TRAPPC2, TRAPPC2L, TRAPPC3, TRAPPC3L, TRAPPC4, TRAPPC5, TRAPPC6A, TRAPPC6B, TRAPPC8, TRAPPC9, TRATI, TRAVI0, TRAV1-1, TRAV1-2, TRAV12-1, TRAV12-2, TRAV12-3, TRAV13-1, TRAV13-2, TRAV14DV4, TRAV16, TRAV17, TRAV18, TRAV19, TRAV2, TRAV20, TRAV21, TRAV22, TRAV23DV6, TRAV24, TRAV25, TRAV26-1, TRAV26-2, TRAV27, TRAV29DV5, TRAV3, TRAV30, TRAV34, TRAV36DV7, TRAV38-1, TRAV38-2DV8, TRAV39, TRAV4, TRAV40, TRAV41, TRAV5, TRAV6, TRAV7, TRAV8-1, TRAV8-2, TRAV8-3, TRAV8-4, TRAV8-6, TRAV8-7, TRAV9-1, TRAV9-2, TRBC2, TRBJ2-1, TRBJ2-2, TRBJ2-2P, TRBJ2-3, TRBJ2-4, TRBJ2-5, TRBJ2-6, TRBJ2-7, TRBV10-1, TRBV10-2, TRBV10-3, TRBV11-1, TRBV19, TRBV2, TRBV20-1, TRBV20OR9-2, TRBV21OR9-2, TRBV23-1, TRBV23OR9-2, TRBV24-1, TRBV25-1, TRBV27, TRBV28, TRBV29-1, TRBV30, TRBV3-1, TRBV4-1, TRBV4-2, TRBV5-1, TRBV5-3, TRBV5-4, TRBV5-5, TRBV5-6, TRBV5-7, TRBV6-1, TRBV6-4, TRBV6-5, TRBV6-6, TRBV6-7, TRBV6-8, TRBV7-1, TRBV7-3, TRBV7-4, TRBV7-6, TRBV7-7, TRBV7-9, TRBV9, TRDC, TRDDI, TRDD2, TRDD3, TRDJ1, TRDJ2, TRDJ3, TRDJ4, TRDMT1, TRDN, TRDV1, TRDV2, TRDV3, TREH, TREM1, TREM2, TREML1, TREML2, TREML4, TRERF1, TREX1, TREX2, TRGC1, TRGC2, TRGJI, TRGJ2, TRGJP, TRGJP1, TRGJP2, TRGVI, TRGV10, TRGV11, TRGV2, TRGV3, TRGV4, TRGV5, TRGV8, TRGV9, TRH, TRHDE, TRHR, TRIAP1, TRIB1, TRIB2, TRIB3, TRIL, TRIM10, TRIM11, TRIM13, TRIM14, TRIM15, TRIM16, TRIM16L, TRIM17, TRIM2, TRIM21, TRIM22, TRIM23, TRIM24, TRIM25, TRIM26, TRIM27, TRIM28, TRIM29, TRIM3, TRIM31, TRIM32, TRIM33, TRIM34, TRIM35, TRIM36, TRIM37, TRIM38, TRIM39, TRIM39-RPP21, TRIM4, TRIM40, TRIM41, TRIM42, TRIM43, TRIM43B, TRIM44, TRIM45, TRIM46, TRIM47, TRIM48, TRIM49, TRIM49B, TRIM49C, TRIM49D1, TRIM49D2, TRIM5, TRIM50, TRIM51, TRIM52, TRIM54, TRIM55, TRIM56, TRIM58, TRIM59, TRIM6, TRIM60, TRIM61, TRIM62, TRIM63, TRIM64, TRIM64B, TRIM64C, TRIM65, TRIM66, TRIM67, TRIM68, TRIM69, TRIM6-TRIM34, TRIM7, TRIM71, TRIM72, TRIM73, TRIM74, TRIM75P, TRIM77, TRIM8, TRIM9, TRIMLI, TRIML2, TRIO, TRIOBP, TRIP10, TRIP11, TRIP12, TRIP13, TRIP4, TRIP6, TRIQK, TRIR, TRITI, TRMO, TRMTI, TRMT10A, TRMTIOB, TRMT10C, TRMT11, TRMT112, TRMT12, TRMT13, TRMTIL, TRMT2A, TRMT2B, TRMT44, TRMT5, TRMT6, TRMT61A, TRMT61B, TRMU, TRNAUIAP, TRNP1, TRNTI, TRO, TROAP, TROVE2, TRPA1, TRPC1, TRPC3, TRPC4, TRPC4AP, TRPC5, TRPC5OS, TRPC6, TRPC7, TRPMI, TRPM2, TRPM3, TRPM4, TRPM5, TRPM6, TRPM7, TRPM8, TRPS1, TRPTI, TRPVI, TRPV2, TRPV3, TRPV4, TRPV5, TRPV6, TRRAP, TRUBI, TRUB2, TSACC, TSCI, TSC2, TSC22D1, TSC22D2, TSC22D3, TSC22D4, TSEN15, TSEN2, TSEN34, TSEN54, TSFM, TSG101, TSGA10, TSGA10IP, TSGA13, TSHB, TSHR, TSHZ1, TSHZ2, TSHZ3, TSKS, TSKU, TSLP, TSN, TSNARE1, TSNAX, TSNAX-DISCI, TSNAXIPI, TSPANI, TSPAN10, TSPAN11, TSPAN12, TSPAN13, TSPAN14, TSPAN15, TSPAN16, TSPAN17, TSPAN18, TSPAN19, TSPAN2, TSPAN3, TSPAN31, TSPAN32, TSPAN33, TSPAN4, TSPAN5, TSPAN6, TSPAN7, TSPAN8, TSPAN9, TSPEAR, TSPO, TSPO2, TSPOAP1, TSPY1, TSPY10, TSPY2, TSPY3, TSPY4, TSPY8, TSPYLI, TSPYL2, TSPYL4, TSPYL5, TSPYL6, TSR1, TSR2, TSR3, TSSC4, TSSKIB, TSSK2, TSSK3, TSSK4, TSSK6, TST, TSTA3, TSTD1, TSTD2, TSTD3, TTBK1, TTBK2, TTC1, TTC12, TTC13, TTC14, TTC16, TTC17, TTC19, TTC21A, TTC21B, TTC22, TTC23, TTC23L, TTC24, TTC25, TTC26, TTC27, TTC28, TTC29, TTC3, TTC30A, TTC30B, TTC31, TTC32, TTC33, TTC34, TTC36, TTC37, TTC38, TTC39A, TTC39B, TTC39C, TTC4, TTC5, TTC6, TTC7A, TTC7B, TTC8, TTC9, TTC9B, TTC9C, TTF1, TTF2, TTI1, TTI2, TTK, TTL, TTLLI, TTLL10, TTLL11, TTLL12, TTLL13P, TTLL2, TTLL3, TTLL4, TTLL5, TTLL6, TTLL7, TTLL8, TTLL9, TTN, TTPA, TTPAL, TTR, TTYHI, TTYH2, TTYH3, TUB, TUBAIA, TUBA1B, TUBAIC, TUBA3C, TUBA3D, TUBA3E, TUBA4A, TUBA4B, TUBA8, TUBAL3, TUBB, TUBB1, TUBB2A, TUBB2B, TUBB3, TUBB4A, TUBB4B, TUBB6, TUBB8, TUBD1, TUBE1, TUBG1, TUBG2, TUBGCP2, TUBGCP3, TUBGCP4, TUBGCP5, TUBGCP6, TUFM, TUFTI, TULPI, TULP2, TULP3, TULP4, TUNAR, TUSCI, TUSC2, TUSC3, TUSC5, TUT1, TVP23A, TVP23B, TVP23C, TVP23C-CDR^{T4}, TWF1, TWF2, TWIST1, TWIST2, TWISTNB, TWNK, TWSGI, TXK, TXLNA, TXLNB, TXLNG, TXN, TXN2, TXNDC11, TXNDC12, TXNDC15, TXNDC16, TXNDC17, TXNDC2, TXNDC5, TXNDC8, TXNDC9, TXNIP, TXNL1, TXNL4A, TXNL4B, TXNRDI, TXNRD2, TXNRD3, TXNRD3NB, TYK2, TYMP, TYMS, TYR, TYRO3, TYROBP, TYRPI, TYSNDI, TYW1, TYWIB, TYW3, TYW5, U2AF1, U2AFIL4, U2AFIL5, U2AF2, U2SURP, UACA, UAPI, UAPILI, UBAI, UBA2, UBA3, UBA5, UBA52, UBA6, UBA7, UBACI, UBAC2, UBALDI, UBALD2, UBAP1, UBAPIL, UBAP2, UBAP2L, UBASH3A, UBASH3B, UBB, UBC, UBD, UBE2A, UBE2B, UBE2C, UBE2D1, UBE2D2, UBE2D3, UBE2D4, UBE2E1, UBE2E2, UBE2E3, UBE2F, UBE2F-SCLY, UBE2G1, UBE2G2, UBE2H, UBE2I, UBE2J1, UBE2J2, UBE2K, UBE2L3, UBE2L5P, UBE2L6, UBE2M, UBE2N, UBE2NL, UBE20, UBE2Q1, UBE2Q2, UBE2Q2L, UBE2QL1, UBE2R2, UBE2S, UBE2T, UBE2U, UBE2V1, UBE2V2, UBE2W, UBE2Z, UBE3A, UBE3B, UBE3C, UBE3D, UBE4A, UBE4B, UBFD1, UBIAD1, UBL3, UBL4A, UBL4B, UBL5, UBL7, UBLCP1, UBN1, UBN2, UBOX5, UBP1, UBQLNI, UBQLN2, UBQLN3, UBQLN4, UBQLNL, UBRI, UBR2, UBR3, UBR4, UBR5, UBR7, UBTD1, UBTD2, UBTF, UBTFL1, UBXN1, UBXN10, UBXN11, UBXN2A, UBXN2B, UBXN4, UBXN6, UBXN7, UBXN8, UCHL1, UCHL3, UCHL5, UCK1, UCK2, UCKL1, UCMA, UCN, UCN2, UCN3, UCP1, UCP2, UCP3, UEVLD, UFC1, UFDI, UFL1, UFM1, UFSP1, UFSP2, UGCG, UGDH, UGGTI, UGGT2, UGP2, UGTIAI, UGTIA10, UGTIA3, UGTIA4, UGTIA5, UGTIA6, UGTIA7, UGT1A8, UGTIA9, UGT2A1, UGT2A2, UGT2A3, UGT2B10, UGT2B11, UGT2B15, UGT2B17, UGT2B28, UGT2B4, UGT2B7, UGT3A1, UGT3A2, UGT8, UHMKI, UHRFI, UHRFIBP1, UHRF1BPIL, UHRF2, UIMCI, ULBP1, ULBP2, ULBP3, ULKI, ULK2, ULK3, ULK4, UMADI, UMOD, UMODLI, UMPS, UNC119, UNC119B, UNC13A, UNC13B, UNC13C, UNC13D, UNC45A, UNC45B, UNC50, UNC5A, UNC5B, UNC5C, UNC5CL, UNC5D, UNC79, UNC80, UNC93A, UNC93B1, UNCX, UNG, UNK, UNKL, UPB1, UPF1, UPF2, UPF3A, UPF3B, UPKIA, UPKIB, UPK2, UPK3A, UPK3B, UPK3BL1, UPP1, UPP2, UPRT, UQCCI, UQCC2, UQCC3, UQCR10, UQCR11, UQCRB, UQCRCI, UQCRC2, UQCRFSI, UQCRH, UQCRHL, UQCRQ, URAD, URB1, URB2, URGCP, URGCP-MRPS24, URI1, URMI, UROC1, UROD, UROS, USB1, USE1, USF1, USF2, USF3, USHIC, USHIG, USH2A, USHBP1, USMG5, USO1, USP1, USP10, USP11, USP12, USP13, USP14, USP15, USP16, USP17L1, USP17L10, USP17L11, USP17L12, USP17L13, USP17L15, USP17L17, USP17L18, USP17L19, USP17L2, USP17L20, USP17L21, USP17L22, USP17L23, USP17L24, USP17L25, USP17L26, USP17L27, USP17L28, USP17L29, USP17L3, USP17L30, USP17L4, USP17L5, USP17L7, USP17L8, USP18, USP19, USP2, USP20, USP21, USP22, USP24, USP25, USP26, USP27X, USP28, USP29, USP3, USP30, USP31, USP32, USP33, USP34, USP35, USP36, USP37, USP38, USP39, USP4, USP40, USP41, USP42, USP43, USP44, USP45, USP46, USP47, USP48, USP49, USP5, USP50, USP51, USP53, USP54, USP6, USP6NL, USP7, USP8, USP9X, USP9Y, USPLI, UST, UTF1, UTP11, UTP14A, UTP14C, UTP15, UTP18, UTP20, UTP23, UTP3, UTP4, UTP6, UTRN, UTS2, UTS2B, UTS2R, UTY, UVRAG, UVSSA, UXSI, UXT, VAC14, VAMP1, VAMP2, VAMP3, VAMP4, VAMP5, VAMP7, VAMP8, VANGLI, VANGL2, VAPA, VAPB, VARS, VARS2, VASHI, VASH2, VASN, VASP, VATI, VATIL, VAVI, VAV2, VAV3, VAX1, VAX2, VBP1, VCAMI, VCAN, VCL, VCP, VCPIP1, VCPKMT, VCX, VCX2, VCX3A, VCX3B, VCY, VCYIB, VDACI, VDAC2, VDAC3, VDR, VEGFA, VEGFB, VEGFC, VEGFD, VENTX, VEPHI, VEZF1, VEZT, VGF, VGLLI, VGLL2, VGLL3, VGLL4, VHL, VHLL, VILI, VILL, VIM, VIP, VIPAS39, VIPRI, VIPR2, VIRMA, VIT, VKORCI, VKORCILI, VLDLR, VMA21, VMAC, VMO1, VMP1, VNIRI, VNIR2, VNIR4, VNIR5, VNN1, VNN2, VNN3, VOPP1, VPREBI, VPREB3, VPS11, VPS13A, VPS13B, VPS13C, VPS13D, VPS16, VPS18, VPS25, VPS26A, VPS26B, VPS28, VPS29, VPS33A, VPS33B, VPS35, VPS36, VPS37A, VPS37B, VPS37C, VPS37D, VPS39, VPS41, VPS45, VPS4A, VPS4B, VPS50, VPS51, VPS52, VPS53, VPS54, VPS72, VPS8, VPS9D1, VRKI, VR^{k2}, VR^{k3}, VRTN, VSIG1, VSIG10, VSIG10L, VSIG10L2, VSIG2, VSIG4, VSIG8, VSIR, VSNL1, VSTMI, VSTM2A, VSTM2B, VSTM2L, VSTM4, VSTM5, VSX1, VSX2, VTA1, VTCNI, VTIIA, VTIIB, VTN, VWA1, VWA2, VWA3A, VWA3B, VWA5A, VWA5B1, VWA5B2, VWA7, VWA8, VWC2, VWC2L, VWCE, VWDE, VWF, WAC, WAPL, WARS, WARS2, WAS, WASFI, WASF2, WASF3, WASHCI, WASHC2A, WASHC2C, WASHC3, WASHC4, WASHC5, WASL, WBP1, WBP11, WBPIL, WBP2, WBP2NL, WBP4, WDCP, WDFYI, WDFY2, WDFY3, WDFY4, WDHD1, WDPCP, WDR1, WDR11, WDR12, WDR13, WDR17, WDR18, WDR19, WDR20, WDR24, WDR25, WDR26, WDR27, WDR3, WDR31, WDR33, WDR34, WDR35, WDR36, WDR37, WDR38, WDR4, WDR41, WDR43, WDR44, WDR45, WDR45B, WDR46, WDR47, WDR48, WDR49, WDR5, WDR53, WDR54, WDR55, WDR59, WDR5B, WDR6, WDR60, WDR61, WDR62, WDR63, WDR64, WDR66, WDR7, WDR70, WDR72, WDR73, WDR74, WDR75, WDR76, WDR77, WDR78, WDR81, WDR82, WDR83, WDR83OS, WDR86, WDR87, WDR88, WDR89, WDR90, WDR91, WDR92, WDR93, WDR97, WDSUB1, WDTC1, WDYHVI, WEEI, WEE2, WFDC1, WFDC10A, WFDC10B, WFDC11, WFDC12, WFDC13, WFDC2, WFDC3, WFDC5, WFDC6, WFDC8, WFDC9, WFIKKNI, WFIKKN2, WFS1, WHAMM, WHRN, WIFI, WIPF1, WIPF2, WIPF3, WIPII, WIPI2, WISP1, WISP2, WISP3, WIZ, WLS, WNKI, WNK2, WNK3, WNK4, WNT1, WNT10A, WNT10B, WNT11, WNT16, WNT2, WNT2B, WNT3, WNT3A, WNT4, WNT5A, WNT5B, WNT6, WNT7A, WNT7B, WNT8A, WNT8B, WNT9A, WNT9B, WRAP53, WRAP73, WRB, WRN, WRNIP1, WSB1, WSB2, WSCD1, WSCD2, WT1, WTAP, WTH3DI, WTIP, WWC1, WWC2, WWC3, WWOX, WWP1, WWP2, WWTR1, XAB2, XAF1, XAGEIA, XAGEIB, XAGE2, XAGE3, XAGE5, XBP1, XCLI, XCL2, XCR1, XDH, XG, XIAP, XIRP1, XIRP2, XK, XKR3, XKR4, XKR5, XKR6, XKR7, XKR8, XKR9, XKRX, XPA, XPC, XPNPEPI, XPNPEP2, XPNPEP3, XPO1, XPO4, XPO5, XPO6, XPO7, XPOT, XPR1, XRCC1, XRCC2, XRCC3, XRCC4, XRCC5, XRCC6, XRN1, XRN2, XRRAI, XXYLTI, XYLB, XYLTI, XYLT2, YAEIDI, YAF2, YAPI, YARS, YARS2, YBEY, YBX1, YBX2, YBX3, YDJC, YEATS2, YEATS4, YES1, YIFIA, YIFIB, YIPF1, YIPF2, YIPF3, YIPF4, YIPF5, YIPF6, YIPF7, YJEFN3, YKT6, YLPMI, YMEILI, YODI, YPELI, YPEL2, YPEL3, YPEL4, YPEL5, YRDC, YTHDC1, YTHDC2, YTHDF1, YTHDF2, YTHDF3, YWHAB, YWHAE, YWHAG, YWHAH, YWHAQ, YWHAZ, YY1, YYIAPI, YY2, Z82206.1, Z83844.1, Z84492.1, Z98749.3, Z98752.3, ZACN, ZADH2, ZAN, ZAP70, ZARI, ZARIL, ZBBX, ZBED1, ZBED2, ZBED3, ZBED4, ZBED5, ZBED6, ZBED6CL, ZBED8, ZBED9, ZBP1, ZBTB1, ZBTB10, ZBTB11, ZBTB12, ZBTB14, ZBTB16, ZBTB17, ZBTB18, ZBTB2, ZBTB20, ZBTB21, ZBTB22, ZBTB24, ZBTB25, ZBTB26, ZBTB3, ZBTB32, ZBTB33, ZBTB34, ZBTB37, ZBTB38, ZBTB39, ZBTB4, ZBTB40, ZBTB41, ZBTB42, ZBTB43, ZBTB44, ZBTB45, ZBTB46, ZBTB47, ZBTB48, ZBTB49, ZBTB5, ZBTB6, ZBTB7A, ZBTB7B, ZBTB7C, ZBTB8A, ZBTB8B, ZBTB8OS, ZBTB9, ZC2HCIA, ZC2HCIB, ZC2HCIC, ZC3H10, ZC3H11A, ZC3H11B, ZC3H12A, ZC3H12B, ZC3H12C, ZC3H12D, ZC3H13, ZC3H14, ZC3H15, ZC3H18, ZC3H3, ZC3H4, ZC3H6, ZC3H7A, ZC3H7B, ZC3H8, ZC3HAVI, ZC3HAVIL, ZC3HC1, ZC4H2, ZCCHC10, ZCCHC11, ZCCHC12, ZCCHC13, ZCCHC14, ZCCHC17, ZCCHC18, ZCCHC2, ZCCHC24, ZCCHC3, ZCCHC4, ZCCHC6, ZCCHC7, ZCCHC8, ZCCHC9, ZCRBI, ZCWPW1, ZCWPW2, ZDBF2, ZDHHC1, ZDHHC11, ZDHHC11B, ZDHHC12, ZDHHC13, ZDHHC14, ZDHHC15, ZDHHC16, ZDHHC17, ZDHHC18, ZDHHC19, ZDHHC2, ZDHHC20, ZDHHC21, ZDHHC22, ZDHHC23, ZDHHC24, ZDHHC3, ZDHHC4, ZDHHC5, ZDHHC6, ZDHHC7, ZDHHC8, ZDHHC9, ZEB1, ZEB2, ZER1, ZFAND1, ZFAND2A, ZFAND2B, ZFAND3, ZFAND4, ZFAND5, ZFAND6, ZFAT, ZFC3H1, ZFHX2, ZFHX3, ZFHX4, ZFP1, ZFP14, ZFP2, ZFP28, ZFP3, ZFP30, ZFP36, ZFP36L1, ZFP36L2, ZFP37, ZFP41, ZFP42, ZFP57, ZFP62, ZFP64, ZFP69, ZFP69B, ZFP82, ZFP90, ZFP91, ZFP91-CNTF, ZFP92, ZFPL1, ZFPM1, ZFPM2, ZFR, ZFR2, ZFX, ZFY, ZFYVEI, ZFYVE16, ZFYVE19, ZFYVE21, ZFYVE26, ZFYVE27, ZFYVE28, ZFYVE9, ZG16, ZG16B, ZGLP1, ZGPAT, ZGRF1, ZHX1, ZHX1-C8orf76, ZHX2, ZHX3, ZICI, ZIC2, ZIC3, ZIC4, ZIC5, ZIKI, ZIM2, ZIM3, ZKSCAN1, ZKSCAN2, ZKSCAN3, ZKSCAN4, ZKSCAN5, ZKSCAN7, ZKSCAN8, ZMAT1, ZMAT2, ZMAT3, ZMAT4, ZMAT5, ZMIZ1, ZMIZ2, ZMPSTE24, ZMYMI, ZMYM2, ZMYM3, ZMYM4, ZMYM5, ZMYM6, ZMYND10, ZMYND11, ZMYND12, ZMYND15, ZMYND19, ZMYND8, ZNF10, ZNF100, ZNF101, ZNF106, ZNF107, ZNF112, ZNF114, ZNF117, ZNF12, ZNF121, ZNF124, ZNF131, ZNF132, ZNF133, ZNF134, ZNF135, ZNF136, ZNF138, ZNF14, ZNF140, ZNF141, ZNF142, ZNF143, ZNF146, ZNF148, ZNF154, ZNF155, ZNF157, ZNF16, ZNF160, ZNF165, ZNF169, ZNF17, ZNF174, ZNF175, ZNF177, ZNF18, ZNF180, ZNF181, ZNF182, ZNF184, ZNF185, ZNF189, ZNF19, ZNF195, ZNF197, ZNF2, ZNF20, ZNF200, ZNF202, ZNF205, ZNF207, ZNF208, ZNF211, ZNF212, ZNF213, ZNF214, ZNF215, ZNF217, ZNF219, ZNF22, ZNF221, ZNF222, ZNF223, ZNF224, ZNF225, ZNF226, ZNF227, ZNF229, ZNF23, ZNF230, ZNF232, ZNF233, ZNF234, ZNF235, ZNF236, ZNF239, ZNF24, ZNF248, ZNF25, ZNF250, ZNF251, ZNF253, ZNF254, ZNF256, ZNF257, ZNF26, ZNF260, ZNF263, ZNF264, ZNF266, ZNF267, ZNF268, ZNF273, ZNF274, ZNF275, ZNF276, ZNF277, ZNF28, ZNF280A, ZNF280B, ZNF280C, ZNF280D, ZNF281, ZNF282, ZNF283, ZNF284, ZNF285, ZNF286A, ZNF286B, ZNF287, ZNF292, ZNF296, ZNF3, ZNF30, ZNF300, ZNF302, ZNF304, ZNF311, ZNF316, ZNF317, ZNF318, ZNF319, ZNF32, ZNF320, ZNF322, ZNF324, ZNF324B, ZNF326, ZNF329, ZNF330, ZNF331, ZNF333, ZNF334, ZNF335, ZNF337, ZNF33A, ZNF33B, ZNF34, ZNF341, ZNF343, ZNF345, ZNF346, ZNF347, ZNF35, ZNF350, ZNF354A, ZNF354B, ZNF354C, ZNF358, ZNF362, ZNF365, ZNF366, ZNF367, ZNF37A, ZNF382, ZNF383, ZNF384, ZNF385A, ZNF385B, ZNF385C, ZNF385D, ZNF391, ZNF394, ZNF395, ZNF396, ZNF397, ZNF398, ZNF404, ZNF407, ZNF408, ZNF41, ZNF410, ZNF414, ZNF415, ZNF416, ZNF417, ZNF418, ZNF419, ZNF420, ZNF423, ZNF425, ZNF426, ZNF428, ZNF429, ZNF43, ZNF430, ZNF431, ZNF432, ZNF433, ZNF436, ZNF438, ZNF439, ZNF44, ZNF440, ZNF441, ZNF442, ZNF443, ZNF444, ZNF445, ZNF446, ZNF449, ZNF45, ZNF451, ZNF454, ZNF460, ZNF461, ZNF462, ZNF467, ZNF468, ZNF469, ZNF470, ZNF471, ZNF473, ZNF474, ZNF479, ZNF48, ZNF480, ZNF483, ZNF484, ZNF485, ZNF486, ZNF487, ZNF488, ZNF490, ZNF491, ZNF492, ZNF493, ZNF496, ZNF497, ZNF500, ZNF501, ZNF502, ZNF503, ZNF506, ZNF507, ZNF510, ZNF511, ZNF512, ZNF512B, ZNF513, ZNF514, ZNF516, ZNF517, ZNF518A, ZNF518B, ZNF519, ZNF521, ZNF524, ZNF525, ZNF526, ZNF527, ZNF528, ZNF529, ZNF530, ZNF532, ZNF534, ZNF536, ZNF540, ZNF541, ZNF543, ZNF544, ZNF546, ZNF547, ZNF548, ZNF549, ZNF550, ZNF551, ZNF552, ZNF554, ZNF555, ZNF556, ZNF557, ZNF558, ZNF559, ZNF559-ZNF177, ZNF560, ZNF561, ZNF562, ZNF563, ZNF564, ZNF565, ZNF566, ZNF567, ZNF568, ZNF569, ZNF57, ZNF570, ZNF571, ZNF572, ZNF573, ZNF574, ZNF575, ZNF576, ZNF577, ZNF578, ZNF579, ZNF580, ZNF581, ZNF582, ZNF583, ZNF584, ZNF585A, ZNF585B, ZNF586, ZNF587, ZNF587B, ZNF589, ZNF592, ZNF593, ZNF594, ZNF595, ZNF596, ZNF597, ZNF598, ZNF599, ZNF600, ZNF605, ZNF606, ZNF607, ZNF608, ZNF609, ZNF610, ZNF611, ZNF613, ZNF614, ZNF615, ZNF616, ZNF618, ZNF619, ZNF620, ZNF621, ZNF622, ZNF623, ZNF624, ZNF625, ZNF625-ZNF20, ZNF626, ZNF627, ZNF628, ZNF629, ZNF630, ZNF638, ZNF639, ZNF641, ZNF644, ZNF645, ZNF646, ZNF648, ZNF649, ZNF652, ZNF653, ZNF654, ZNF655, ZNF658, ZNF66, ZNF660, ZNF662, ZNF664, ZNF665, ZNF667, ZNF668, ZNF669, ZNF670, ZNF670-ZNF695, ZNF671, ZNF672, ZNF674, ZNF675, ZNF676, ZNF677, ZNF678, ZNF679, ZNF680, ZNF681, ZNF682, ZNF683, ZNF684, ZNF687, ZNF688, ZNF689, ZNF69, ZNF691, ZNF692, ZNF695, ZNF696, ZNF697, ZNF699, ZNF7, ZNF70, ZNF700, ZNF701, ZNF703, ZNF704, ZNF705A, ZNF705B, ZNF705D, ZNF705E, ZNF705G, ZNF706, ZNF707, ZNF708, ZNF709, ZNF71, ZNF710, ZNF711, ZNF713, ZNF714, ZNF716, ZNF717, ZNF718, ZNF720, ZNF721, ZNF724, ZNF726, ZNF727, ZNF728, ZNF729, ZNF730, ZNF732, ZNF735, ZNF736, ZNF737, ZNF738, ZNF74, ZNF740, ZNF746, ZNF747, ZNF749, ZNF750, ZNF75A, ZNF75D, ZNF76, ZNF761, ZNF763, ZNF764, ZNF765, ZNF766, ZNF768, ZNF77, ZNF770, ZNF771, ZNF772, ZNF773, ZNF774, ZNF775, ZNF776, ZNF777, ZNF778, ZNF780A, ZNF780B, ZNF781, ZNF782, ZNF783, ZNF784, ZNF785, ZNF786, ZNF787, ZNF788, ZNF789, ZNF79, ZNF790, ZNF791, ZNF792, ZNF793, ZNF799, ZNF8, ZNF80, ZNF800, ZNF804A, ZNF804B, ZNF805, ZNF808, ZNF81, ZNF813, ZNF814, ZNF816, ZNF816-ZNF321P, ZNF821, ZNF823, ZNF827, ZNF829, ZNF83, ZNF830, ZNF831, ZNF835, ZNF836, ZNF837, ZNF839, ZNF84, ZNF841, ZNF843, ZNF844, ZNF845, ZNF846, ZNF85, ZNF850, ZNF852, ZNF853, ZNF860, ZNF862, ZNF865, ZNF878, ZNF879, ZNF880, ZNF883, ZNF888, ZNF891, ZNF90, ZNF91, ZNF92, ZNF93, ZNF98, ZNF99, ZNFX1, ZNHIT1, ZNHIT2, ZNHIT3, ZNHIT6, ZNRD1, ZNRF1, ZNRF2, ZNRF3, ZNRF4, ZP1, ZP2, ZP3, ZP4, ZPBP, ZPBP2, ZPLDI, ZPRI, ZRANB1, ZRANB2, ZRANB3, ZRSRI, ZRSR2, ZSCANI, ZSCAN10, ZSCAN12, ZSCAN16, ZSCAN18, ZSCAN2, ZSCAN20, ZSCAN21, ZSCAN22, ZSCAN23, ZSCAN25, ZSCAN26, ZSCAN29, ZSCAN30, ZSCAN31, ZSCAN32, ZSCAN4, ZSCAN5A, ZSCAN5B, ZSCAN5C, ZSCAN9, ZSWIM1, ZSWIM2, ZSWIM3, ZSWIM4, ZSWIM5, ZSWIM6, ZSWIM7, ZSWIM8, ZUFSP, ZW10, ZWILCH, ZWINT, ZXDA, ZXDB, ZXDC, ZYG11A, ZYG11B, ZYX, ZZEF1, and ZZZ3.

### Protein Level Control

This description also provides methods for the control of protein levels with a cell. This is based on the use of compounds as described herein, which are known to interact with a specific target protein such that degradation of a target protein in vivo will result in the control of the amount of protein in a biological system, preferably to a particular therapeutic benefit.

Furthermore, the invention provides the use of a compound according to the definitions herein, or a pharmaceutically acceptable salt, or a hydrate or solvate thereof for the preparation of a medicament for the treatment of an autoimmune disorder, an inflammatory disorder, or a proliferative disorder, or a disorder commonly occurring in connection with transplantation.

### Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 R^{A}, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, arc administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In one embodiment, the present invention provides a composition comprising a provided compound and one or more additional therapeutic agents. The therapeutic agent may be administered together with a provided compound or may be administered prior to or following administration of a provided compound. Suitable therapeutic agents are described in further detail below. In certain embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating an inflammatory disease, disorder or condition by administering to a patient in need thereof a provided compound and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (AralcnR), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (LeukeranR), cyclosporine (Sandimmune®), leflunomide (Arava®) and “anti-TNF” agents such as etanercept (EnbrelR), infliximab (Remicade®), golimumab (SimponiR), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (OrenciaR), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (AmitizaR), laxatives such as Milk of Magnesia, polyethylene glycol (MiraLax®), Dulcolax®, Correctol® and SenokotR, anticholinergics or antispasmodics such as dicyclomine (Bentyl®), SingulairR, beta-2 agonists such as albuterol (Ventolin®) HFA, Proventil® HFA), levalbuterol (Xopenex®), metaproterenol (Alupent®), pirbuterol acetate (Maxair®), terbutaline sulfate (BrethaireR), salmeterol xinafoate (SereventR) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide

(Aerobid®), Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (VidexR), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (IntelenceR), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron R) in combination with lenalidomide (Revlimid R), or any combination(s) thereof.

In another embodiment, the present invention provides a method of treating rheumatoid arthritis comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or CuprimineR), azathioprine (Imuran®), cyclophosphamide (Cytoxan®), chlorambucil (Leukeran (R), cyclosporine (SandimmuneR), leflunomide (Arava®) and “anti-TNF” agents such as etanercept (EnbrelR), infliximab (Remicade®), golimumab (SimponiR), certolizumab pegol (Cimzia®) and adalimumab (HumiraR), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present invention provides a method of treating systemic lupus erythematosus comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present invention provides a method of treating Crohn's disesase, ulcerative colitis, or inflammatory bowel disease comprising administering to a patient in need thereof a provided compound and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a provided compound and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (IntalR), methylxanthines such as theophylline (Theo-Dur®, Theolair®, Slo-bid®, Uniphyl®, Theo-24®) and aminophylline, and IgE antibodies such as omalizumab (Xolair®).

In some embodiments, the present invention provides a method of treating COPD comprising administering to a patient in need thereof a provided compound and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (AtroventR) and tiotropium (Spiriva®), methylxanthines such as theophylline (Theo-Dur®, TheolairR, Slo-bid®, Uniphyl®, Theo-24®) and aminophylline, inhaled corticosteroids such as prednisone, prednisolone, beclomethasone dipropionate (BecloventR, QvarR, and Vanceril®), triamcinolone acetonide (Azmacort®), mometasone (Asthmanex®), budesonide (Pulmocort®), flunisolide (Aerobid®), Afviar®, Symbicort®, and Dulera®,
In another embodiment, the present invention provides a method of treating a hematological malignancy comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PI3K inhibitor, a SYK inhibitor, and combinations thereof.

In another embodiment, the present invention provides a method of treating a solid tumor comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PI3K inhibitor, a SYK inhibitor, and combinations thereof.

In another embodiment, the present invention provides a method of treating a hematological malignancy comprising administering to a patient in need thereof a provided compound and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Lcuk. Rcs. (2012), published online July 17, and incorporated herein by reference in its entirety).

In another embodiment, the present invention provides a method of treating diffuse large B-cell lymphoma (DLBCL) comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, the present invention provides a method of treating multiple myeloma comprising administering to a patient in need thereof a provided compound and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating or lessening the severity of a disease comprising administering to a patient in need thereof a provided compound and a BTK inhibitor, wherein the disease is selected from inflammatory bowel disease, arthritis, systemic lupus erythematosus (SLE), vasculitis, idiopathic thrombocytopenia purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenia purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenia states, Goodpasture's syndrome, atherosclerosis, Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenia purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleraderma, mycosis fungoides, acute inflammatory responses (such as acute respiratory distress syndrome and ischemia/reperfusion injury), and Graves' discasc.

In another embodiment, the present invention provides a method of treating or lessening the severity of a disease comprising administering to a patient in need thereof a provided compound and a PI3K inhibitor, wherein the disease is selected from a cancer, a neurodegenative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a provided compound and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments the present invention provides a method of treating or lessening the severity of a disease comprising administering to a patient in need thereof a provided compound and a Bcl-2 inhibitor, wherein the disease is an inflammatory disorder, an autoimmune disorder, a proliferative disorder, an endocrine disorder, a neurological disorder, or a disorder associated with transplantation. In some embodiments, the disorder is a proliferative disorder, lupus, or lupus nephritis. In some embodiments, the proliferative disorder is chronic lymphocytic leukemia, diffuse large B-cell lymphoma, Hodgkin's disease, small-cell lung cancer, non-small-cell lung cancer, myelodysplastic syndrome, lymphoma, a hematological neoplasm, or solid tumor.

A compound of the current invention may also be used to advantage in combination with other therapeutic compounds. In some embodiments, the other therapeutic compounds are antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antincoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin. The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name AromasinTM. Formestane is marketed under the trade name LentaronTM. Fadrozole is marketed under the trade name Afema™ Anastrozole is marketed under the trade name ArimidexTM. Letrozole is marketed under the trade names Femara™ or FemarTM. Aminoglutethimide is marketed under the trade name Orimeten™ A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name NolvadexTM. Raloxifene hydrochloride is marketed under the trade name Evista™ Fulvestrant can be administered under the trade name FaslodexTM. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name ZoladexTM.

The term “topoisomerase I inhibitor” as used herein includes, but is not limited to topotccan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark CamptosarTM. Topotecan is marketed under the trade name HycamptinTM.

The term “topoisomerase II inhibitor” as used herein includes, but is not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophillotoxines etoposide and teniposide. Etoposide is marketed under the trade name EtopophosTM. Teniposide is marketed under the trade name VM 26-Bristol Doxorubicin is marketed under the trade name Acriblastin™ or Adriamycin™. Epirubicin is marketed under the trade name FarmorubicinTM. Idarubicin is marketed under the trade name ZavedosTM. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name TaxolTM. Docetaxel is marketed under the trade name Taxotere™. Vinblastine sulfate is marketed under the trade name Vinblastin R.PTM. Vincristine sulfate is marketed under the trade name Farmistin™

The term “alkylating agent” as used herein includes, but is not limited to, cyclophosphamide, ifosfamide, melphalan or nitrosourea (BCNU or Gliadel). Cyclophosphamide is marketed under the trade name Cyclostin™ Ifosfamide is marketed under the trade name Holoxan™

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name XelodaTM. Gemcitabine is marketed under the trade name GemzarTM.

The term “platin compound” as used herein includes, but is not limited to, carboplatin, cis -platin, cisplatinum and oxaliplatin. Carboplatin can be administered, e.g., in the form as it is marketed, e.g. under the trademark CarboplatTM. Oxaliplatin can be administered, e.g., in the form as it is marketed, e.g. under the trademark Eloxatin™

The term “compounds targeting/decreasing a protein or lipid kinase activity; or a protein or lipid phosphatase activity; or further anti-angiogenic compounds” as used herein includes, but is not limited to, protein tyrosine kinase and/or scrinc and/or threonine kinase inhibitors or lipid kinase inhibitors, such as a) compounds targeting, decreasing or inhibiting the activity of the platelet-derived growth factor —receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor -receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; Ilmofosine; RO 318220 and RO 320432; GO 6976; Isis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinascs (EGFR1 ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, C1-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; scc WO2008118802), navitoclax (and analogs thereof, scc U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

Further examples of BTK inhibitory compounds, and conditions treatable by such compounds in combination with compounds of this invention can be found in WO2008039218, U.S. Pat. No. 7,514,444, WO2011090760, and U.S. Pat. No. 8,338,439, the entirety of each of which is herein incorporated by reference.

Further examples of SYK inhibitory compounds, and conditions treatable by such compounds in combination with compounds of this invention can be found in WO2003063794, U.S. Pat. No. 7,557,210, WO2005007623, U.S. Pat. No. 7,173,015, WO2006078846, and U.S. Pat. No. 7,449,458, the entirety of each of which is herein incorporated by reference.

Further examples of PI3K inhibitory compounds, and conditions treatable by such compounds in combination with compounds of this invention can be found in WO2004019973, U.S. Pat. No. 7,713,943, WO2004089925, U.S. Pat. No. 6,949,537, WO2007016176, U.S. Pat. Nos. 7,402,325, 8,138,347, WO2002088112, U.S. Pat. No. 7,071,189, WO2007084786, U.S. Pat. No. 8,217,035, WO2007129161, U.S. Pat. No. 7,781,433, WO2006122806, U.S. Pat. No. 7,667,039, WO2005113554, U.S. Pat. No. 7,932,260, WO2007044729, and U.S. Pat. No. 7,989,622, the entirety of each of which is herein incorporated by reference.

Further examples of JAK inhibitory compounds, and conditions treatable by such compounds in combination with compounds of this invention can be found in WO2009114512, U.S. Pat. No. 8,185,616, WO2008109943, U.S. Pat. No. 8,486,941, WO2007053452, U.S. Pat. No. 7,528,143, WO200142246, U.S. Pat. No. 6,627,754, WO2007070514, and U.S. Pat. No. 7,598,257, the entirety of each of which is herein incorporated by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α-γ- or δ-tocopherol or α-γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name DidronelTM. Clodronic acid is marketed under the trade name BonefosTM. Tiludronic acid is marketed under the trade name SkelidTM. Pamidronic acid is marketed under the trade name Aredia™ Alendronic acid is marketed under the trade name Fosamax™ Ibandronic acid is marketed under the trade name BondranatTM. Risedronic acid is marketed under the trade name ActonelTM. Zoledronic acid is marketed under the trade name ZometaTM. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-B-D-arabinofuransylcytosine (ara-c) and bisulfan; ALK inhibitors, which are compounds which target, decrease or inhibit anaplastic lymphoma kinase, and Bcl-2 inhibitors.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino, 17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412. In some embodiments, the present invention provides a method of treating AML associated with an ITD and/or D835Y mutation, comprising administering a compound of the present invention together with a one or more FLT3 inhibitors. In some embodiments, the FLT3 inhibitors are selected from quizartinib (AC220), a staurosporine derivative (e.g. midostaurin or lestaurtinib), sorafenib, tandutinib, LY-2401401, LS-104, EB-10, famitinib, NOV-110302, NMS-P948, AST-487, G-749, SB-1317, S-209, SC-110219, AKN-028, fedratinib, tozasertib, and sunitinib. In some embodiments, the FLT3 inhibitors are selected from quizartinib, midostaurin, lestaurtinib, sorafenib, and sunitinib.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylasc (HDAC) inhibitors such as sodium butyrate and subcroylanilidc hydroxamic acid (SAHA) inhibit the activity of the enzymes known as histone deacetylases. Specific

HDAC inhibitors include MS275, SAHA, FK228 (formerly FR901228), Trichostatin A and compounds disclosed in U.S. Pat. No. 6,552,065 including, but not limited to, N-hydroxy-3-[4-[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl) {2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4i th Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl) phthalazine or a pharmaceutically acceptable salt thereof, 1-(4-chloroanilino)-4-(4-pyridylmethyl) phthalazine succinate; AngiostatinTM; EndostatinTM; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-a-epihydrocotisol, cortexolone, 17a-hydroxyprogesterone, corticosterone, desoxycorticosterone, testosterone, estrone and dexamethasone.

Implants containing corticosteroids refers to compounds, such as fluocinolone and dexamcthasonc.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (ArifloR GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICIDTMCC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, and Takeda antagonists such as N-[[4-[6,7-dihydro-2-(4-methylphenyl)-5H-benzo-cyclohepten-8-yl] carbonyl]amino]phenyl]-methyl] tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of one or more other therapeutic agent present in the compositions of this invention may be no more than the amount that would normally be administered in a composition comprising that therapeutic agent as the only active agent. Preferably the amount of one or more other therapeutic agent in the presently disclosed compositions will range from about 50% to 100% of the amount normally present in a composition comprising that agent as the only therapeutically active agent. In some embodiments, one or more other therapeutic agent is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is approvided for dosing per the FDA label insert.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-HI (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILRI/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTBR, LIGHT, DcR3, HVEM, VEGI/TLIA, TRAMP/DR3, EDAR, EDAI, XEDAR, EDA2, TNFRI, Lymphotoxin a/TNFB, TNFR2, TNFa, LTBR, Lymphotoxin alß2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L¹, PD-L², LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonists of KIR, such as lirilumab.

In some embodiments, an immuno-oncology agent is an agent that inhibits or depletes macrophages or monocytes, including but not limited to CSF-IR antagonists such as CSF-IR antagonist antibodies including RG7155 (WO 2011/070024, US 2011/0165156, WO 2011/0107553, US 2012/0329997, WO 2011/131407, US 2013/0005949, WO 2013/087699, US 2014/0336363, WO 2013/119716, WO 2013/132044, US 2014/0079706) or FPA-008 (WO 2011/140249, US 2011/0274683; WO 2013/169264; WO 2014/036357, US 2014/0079699).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO 2010/077634, US 2010/0203056), durvalumab (MEDI4736), BMS-936559 (WO 2007/005874, US 2009/0055944), and MSB0010718C (WO 2013/079174, US 2014/0341917).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO 2010/019570, US 2010/0150892, WO 2014/008218, US 2014/0093511), or IMP-731 or IMP-321 (WO 2008/132601, US 2010/0233183, WO 2009/044273, US 2011/0008331).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO 2006/105021, US 2007/0098719, WO 2009/009116, US 2009/0136494), or MK-4166 (WO 2011/028683, US 2012/0189639).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS: F001287 (Bristol-Myers Squibb); Phy906/KD108 (Phytoceutica); an enzyme that breaks down kynurenine (Kynase, Kyn Therapeutics); and NLG-919 (WO 2009/073620, US 2011/0053941, WO 2009/132238, US 2011/0136796, WO 2011/056652, US 2012/0277217, WO 2012/142237, US 2014/0066625).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO 2006/029879, U.S. Pat. No. 7,501,496).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3)(WO 2011/109400, US 2013/0149236).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor -reactive T cells and have been shown in clinical trials to induce durable anti-tumor responses in increasing numbers of tumor histologics, including some tumor types that conventionally have not been considered immunotherapy sensitive. See, e.g., Okazaki, T. et al. (2013)Nat. Immunol. 14, 1212-1218; Zou et al. (2016) Sci. Transl. Med. 8. The anti-PD-1 antibody nivolumab (Opdivo®, Bristol-Myers Squibb, also known as ONO—4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In some embodiments, an immuno-oncology agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (Pexa Vec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC)(NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jenncrex Biotherapeutics), a TK- and vaccinia growth factor -deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFa-IRES-hIL20; and VSV-GP(ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682, the entirety of each of which is herein incorporated by reference, which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor -related orphan receptor γ (RORγt). RORγt is a transcription factor with key roles in the differentiation and maintenance of Type 17 effector subsets of CD4+ (Th17) and CD8+ (Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that may be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15: sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams ET. AL., “Big opportunities for small molecules in immuno-oncology,” Cancer Therapy 2015, Vol. 14, pages 603-622, the content of which is incorporated herein by reference in its entirety. In some embodiment, an immuno-oncology agent is selected from the examples described in Table 1 of Jerry L. Adams ET. AL. In some embodiments, an immuno-oncology agent is a small molecule targeting an immuno-oncology target selected from those listed in Table 2 of Jerry L. Adams ET. AL. In some embodiments, an immuno-oncology agent is a small molecule agent selected from those listed in Table 2 of Jerry L. Adams ET. AL.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BiTER) antibody constructs can mediate bystander tumor cell killing”, PLOS ONE 12 (8): e0183390, the content of which is incorporated herein by reference in its entirety. In some embodiments, an immuno-oncology agent is a bispecific T cell engager (BiTE®) antibody construct. In some embodiments, a bispecific T cell engager (BiTER) antibody construct is a CD19/CD3 bispecific antibody construct. In some embodiments, a bispecific T cell engager (BiTER) antibody construct is an EGFR/CD3 bispecific antibody construct. In some embodiments, a bispecific T cell engager (BiTER) antibody construct activates T cells. In some embodiments, a bispecific T cell engager (BiTER) antibody construct activates T cells, which release cytokines inducing upregulation of intercellular adhesion molecule 1 (ICAM-1) and FAS on bystander cells. In some embodiments, a bispecific T cell engager (BiTER) antibody construct activates T cells which result in induced bystander cell lysis. In some embodiments, the bystander cells are in solid tumors. In some embodiments, the bystander cells being lysed are in proximity to the BiTER-activated T cells. In some embodiment, the bystander cells comprises tumor -associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor -infiltrating T cell. In some embodiments, an immuno-oncology agent is a bispecific antibody construct or chimeric antigen receptors (CARs) that directly connect T cells with tumor -associated surface antigens (TAAs).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should procced.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDLI, PDL2, PDI, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDLI, PDL2, PDI, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8+ (aß) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-LI monoclonal Antibody (Anti-B7-HI; MEDI4736), MK-3475 (PD-1 blocker), Nivolumab (anti-PDI antibody), CT-011 (anti-PDI antibody), BY55 monoclonal antibody, AMP224 (anti-PDLI antibody), BMS-936559 (anti-PDLI antibody), MPLDL3280A (anti-PDLI antibody), MSB0010718C (anti-PDLI antibody), and ipilimumab (anti-CTLA-4 checkpoint inhibitor). Checkpoint protein ligands include, but are not limited to PD-LI, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (Opdivo®), ipilimumab (Yervoy®), and pembrolizumab (KeytrudaR). In some embodiments, the checkpoint inhibitor is selected from nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); and atezolizumab (anti-PD-L1 antibody, TecentriqR, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (Keytruda®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that may be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981).

Checkpoint inhibitors that may be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute mycloid leukemia (NCT02678338) and lymphoma (NCT02953509).

Checkpoint inhibitors that may be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that may be used in the present invention include CSFIR inhibitors. CSFIR inhibitors that are being studied in clinical trials include pexidartinib (PLX3397, Plexxikon), a CSFIR small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2 ((1R,2R)-2-hydroxycyclohexylamino)-benzothiazol-6-yloxyl]-pyridine-2-carboxylic acid methylamide, Novartis), an orally available inhibitor of CSFIR, in advanced solid tumors (NCT02829723).

Checkpoint inhibitors that may be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### Exemplification

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

All reactions are carried out under nitrogen or argon unless otherwise stated.

Proton NMR (1H NMR) is conducted in deuterated solvent. In certain compounds disclosed herein, one or more 1H shifts overlap with residual proteo solvent signals; these signals have not been reported in the experimental provided hereinafter.

TABLE 3
Analytical instruments

| | |
|---|---|
| LCMS | Shimadzu UFLC MS: LCMS-2020 |
| | Agilent Technologies 1200 series MS: Agilent Technologies 6110 |
| | Agilent Technologies 1200 series MS: LC/MSD VL |
| NMR | BRUKER AVANCE III/400; Frequency (MHz) 400.13; Nucleus: 1H; |
| | Number of Transients: 8 |
| Prep-HPLC | Gilson GX-281 systems: instruments GX-A, GX-B, GX-C, GX-D, GX-E, |
| | GX-F, GX-G and GX-H |
| GCMS | SHIMADZU GCMS-QP2010 Ultra |
| Analytical cSFC | Agilent Technologies 1290 Infinity |
| Prep-cSFC | Waters SFC Prep 80 |

For acidic LCMS data: LCMS is recorded on an Agilent 1200 Series LC/MSD or Shimadzu LCMS2020 equipped with electro-spray ionization and quadruple MS detector [ES+ve to give MH+] and equipped with Chromolith Flash RP-18e 25*2.0 mm, eluting with 0.0375 vol % TFA in water (solvent A) and 0.01875 vol % TFA in acetonitrile (solvent B). Other LCMS is recorded on an Agilent 1290 Infinity RRLC attached with Agilent 6120 Mass detector. The column used is BEH C18 50*2.1 mm, 1.7 micron. Column flow is 0.55 ml/min and mobile phase used is (A)₂ mM Ammonium Acetate in 0.1l% Formic Acid in Water and (B)_{0.1}% Formic Acid in Acetonitrile.

For basic LCMS data: LCMS is recorded on an Agilent 1200 Series LC/MSD or Shimadzu LCMS 2020 equipped with electro-spray ionization and quadruple MS detector [ES+ve to give MH+] and equipped with Xbridge C18, 2.1X50 mm columns packed with 5 mm C18-coated silica or Kinetex EVO C18 2.1X30 mm columns packed with 5 mm C18-coated silica, eluting with 0.05 vol % NH₃H2O in water (solvent A) and acetonitrile (solvent B).

HPLC Analytical Method: HPLC is carried out on X Bridge C18 150*4.6 mm, 5 micron. Column flow is 1.0 ml/min and mobile phase used is (A)0.1% Ammonia in water and (B)0.1% Ammonia in Acetonitrile.

Prep HPLC Analytical Method: The compound is purified on Shimadzu LC-20AP and UV detector. The column used is X-BRIDGE C18 (250*19) mm, 5u. Column flow is 16.0 ml/min. Mobile phase is (A)0.1% Formic Acid in Water and (B) Acetonitrile. Basic method used is (A)5 mM ammonium bicarbonate and 0.1% NH₃ in Water and (B) Acetonitrile or (A)0.1% Ammonium Hydroxide in Water and (B) Acetonitrile. The UV spectra are recorded at 202 nm & 254 nm.

NMR Method: The 1H NMR spectra are recorded on a Bruker Ultra Shield Advance 400 MHz/5 mm Probe (BBFO). The chemical shifts are reported in part-per-million.

### Intermediates:

### Ethyl 2-(5-amino-2-oxo-1-pyridyl)acetate (Intermediate A

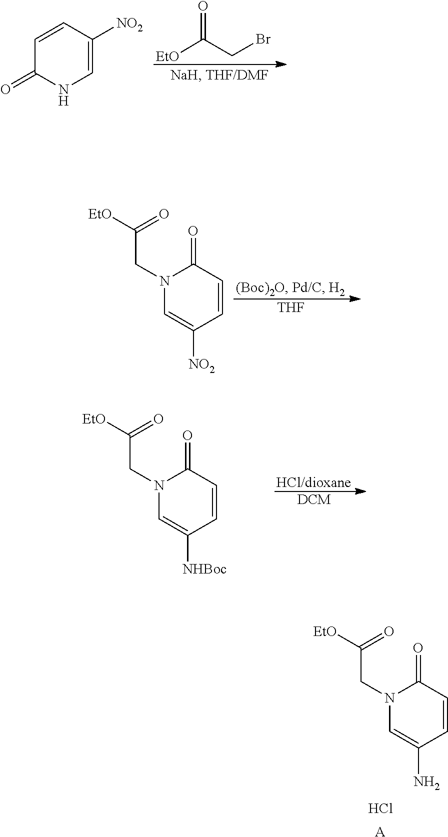

Step 1-Ethyl 2-(5-nitro-2-oxo-1-pyridyl)acetate. To a solution of 5-nitro-1H-pyridin-2-one (5.00 g, 35.7 mmol, CAS #5418-51-9) in THF (70 mL) and DMF (10 mL) was added NaH (1.71 g, 42.8 mmol, 60% dispersion in mineral oil), then the mixture was stirred at 0° C. for 1 hr. Next, ethyl 2-bromoacetate (6.56 g, 39. mmol, CAS #105-36-2) was added, and the mixture was stirred at 25° C. for 1 hr. On completion, saturated NH₄Cl was added to the mixture to adjust the pH to 6. Then the mixture was diluted with water (20 mL), filtered to give the filter cake and dried in vacuo. The crude product was purified by column chromatography (SiO₂, PE: EA=1:0 to 0:1) to give the title compound (6.20 g, 77% yield) as white solid. 1H NMR (400 MHz, DMSO-d6) δ 9.25 (d, J=3.2 Hz, 1H), 8.20-8.10 (m, 1H), 6.55 (d, J=10.0 Hz, 1H), 4.86 (s, 2H), 4.17 (q, J=7.2 Hz, 2H), 1.21 (t, J=7.2 Hz, 3H); LC-MS (ESI+) m/z 226.9 (M+H)+

Step 2-Ethyl 2-[5-(tert-butoxycarbonylamino)-2-oxo-1-pyridyl|acetate. To a solution of ethyl 2-(5-nitro-2-oxo-1-pyridyl)acetate (1 g, 4.42 mmol) in THF (10 mL) was added (Boc)₂O (2.89 g, 13.2 mmol) and Pd/C (1 g, 4.42 mmol, 10 wt %) under N₂. The suspension was degassed under vacuum and purged with H₂ three times. The mixture was stirred under H₂ (15 psi) at 25° C. for 2 hours. On completion, the reaction mixture was filtered and concentrated in vacuo to give the residue. The residue was purified by column chromatography (SiO₂, PE/EA=20/1 to 1/1) to give the title compound (985 mg, 75% yield) as red solid. 1H NMR (400 MHZ, DMSO-d6) δ 9.05 (s, 1H), 7.87 (s, 1H), 7.39 (m, 1H), 6.39 (d, J=9.6 Hz, 1H), 4.67 (s, 2H), 4.13 (q, J=7.2 Hz, 2H), 1.45 (s, 9H), 1.20 (t, J=7.2 Hz, 3H); LC-MS (ESI+) m/z 297.0 (M+H)+.

Step 3-Ethyl 2-(5-amino-2-oxo-1-pyridyl)acetate. To a solution of ethyl 2-[5-(tert-butoxycarbonylamino)-2-oxo-1-pyridyl] acetate (500 mg, 1.69 mmol) in DCM (5 mL) was added HCl/dioxane (4 M, 662 uL), then the mixture was stirred at 25° C. for 10 hrs. On completion, the reaction mixture was concentrated in vacuo to give the title compound (330 mg, 84% yield, HCl) as yellow solid. LC-MS (ESI+) m/z 197.1 (M+H)+.

(3S)-1-(3-Pyridyl) piperidine-3-carboxylic acid (Intermediate B)

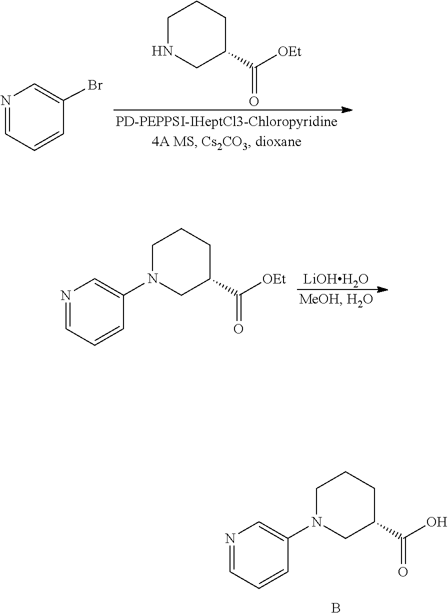

Step 1-Ethyl (3S)-1-(3-pyridyl) piperidine-3-carboxylate. To a mixture of 3-bromopyridine (1.00 g, 6.33 mmol, CAS #626-55-1), ethyl (3S)-piperidine-3-carboxylate (1.19 g, 7.60 mmol, CAS #37675-18-6), 4 Å molecular sieves (100 mg) and Cs2CO₃ (6.19 g, 18.9 mmol) in dioxane (10 mL) was added 1,3-bis [2,6-bis(1-propylbutyl)phenyl]-4,5-dichloro-2H-imidazol-1-ium-2-ide;3-chloropyridine;dichloropalladium (123 mg, 127 umol). The mixture was purged with N₂ three times, then the mixture was stirred at 100° C. for 16 hrs under N₂ atmosphere. On completion, the reaction mixture was diluted with H₂O (40 mL) and extracted with DCM (100 mL×3). The combined organic layers were washed with saturated NaCl (20 mL), dried over Na₂SO₄, filtered and concentrated in vacuo to give a residue. The residue was purified by column chromatography (SiO₂, PE: EA=10:1 to 1:1) to give the title compound (1.40 g, 91% yield, 98% ee) as light yellow liquid. 1H NMR (400 MHZ, DMSO-d6) δ 8.29 (s, 1H), 7.97 (s, 1H), 7.33-7.26 (m, 1H), 7.20-7.11 (m, 1H), 4.11-4.00 (m, 2H), 3.66-3.54 (m, 1H), 3.50-3.44 (m, 1H), 3.08-3.00 (m, 1H), 2.93-2.85 (m, 1H), 2.67-2.59 (m, 1H), 1.94-1.86 (m, 1H), 1.75-1.52 (m, 3H), 1.22-1.15 (m, 3H); LC-MS (ESI+) m/z 235.6 (M+H)+.

Step 2-(3S)-1-(3-pyridyl) piperidine-3-carboxylic acid. To a solution of ethyl (3S)-1-(3-pyridyl) piperidine-3-carboxylate (830 mg, 3.54 mmol) in MeOH (4 mL) and H₂O (1 mL) was added LiOH.H2O (595 mg, 14.2 mmol). Then the mixture was stirred at 25° C. for 1 hr. On completion, the reaction mixture was acidified with HCl (4 N) until the pH=5, then concentrated in vacuo to give the title compound (730 mg, 100% yield) as yellow solid. 1H NMR (400 MHZ, D₂O) δ 8.29-8.23 (m, 1H), 8.08-7.99 (m, 2H), 7.79-7.70 (m, 1H), 3.85-3.77 (m, 1H), 3.58-3.54 (m, 1H), 3.50-3.41 (m, 1H), 3.25-3.12 (m, 1H), 2.86-2.75 (m, 1H), 2.08-1.99 (m, 1H), 1.91-1.75 (m, 2H), 1.73-1.61 (m, 1H); LC-MS (ESI+) m/z 206.9 (M+H)+.

### Ethyl 2-(5-amino-4-chloro-2-oxo-1-pyridyl)acetate (Intermediate C

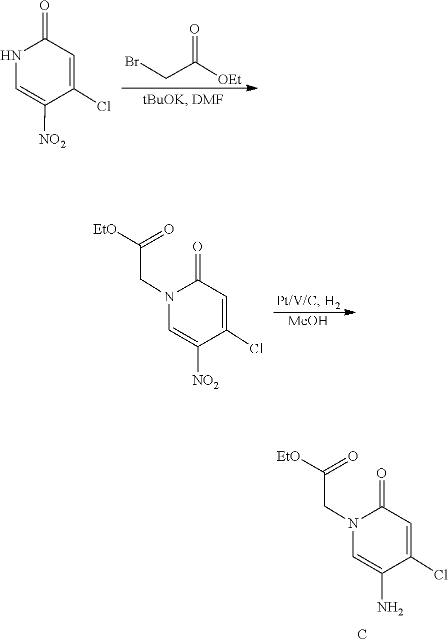

Step 1-Ethyl 2-(4-chloro-5-nitro-2-oxo-1-pyridyl)acetate. To a solution of 4-chloro-5-nitro-1H-pyridin-2-one (2.00 g, 11.4 mmol, CAS #850663-54-6) in DMF (15 mL) was added tBuOK (1.93 g, 17.1 mmol) at 0° C., the mixture was stirred at rt for 30 min. Next, ethyl 2-bromoacetate (2.49 g, 14.9 mmol) was added slowly at 0° C., then reaction mixture was stirred at rt for 3 hrs. On completion, the reaction mixture was quenched with HOAc (3 mL) and diluted with EA (150 mL). The organic layer was washed with H₂O (70 mL×4), dried over Na₂SO₄, filtered and concentrated in vacuo. The residue was purified by column chromatography (SiO₂, PE: EA=3:1,PE: EA=1:1,P1: R (=0.4) to give the title compound (1.00 g, 33% yield) as yellow solid. 1H NMR (400 MHZ, CDC13)δ 8.55 (s, 1H), 6.74 (s, 1H), 4.72 (s, 2H), 4.30 (q, J=7.2 Hz, 2H), 1.33 (t, J=7.2 Hz, 3H).

Step 2-Ethyl 2-(5-amino-4-chloro-2-oxo-1-pyridyl)acetate. To a solution of ethyl 2-(4-chloro-5-nitro-2-oxo-1-pyridyl)acetate (200 mg, 767 umol) in MeOH (20 mL) was added Pt/V/C (200 mg, 767 umol) under N₂. The suspension was degassed under vacuo and purged with H₂ several times. The mixture was then stirred under H₂ (15 psi) at rt for 2 hrs. On completion, the reaction mixture was filtered and the filtrate was concentrated in vacuo to give the title compound (170 mg, 96% yield) as brown oil. 1H NMR (400 MHZ, DMSO-d6) δ 7.13 (s, 1H), 6.58 (s, 1H), 4.62 (s, 2H), 4.15-4.11 (m, 2H), 3.47 (s, 2H), 1.21-1.18 (m, 3H).

### 4-Bromo-5-nitro-1H-pyridin-2-one (Intermediate D)

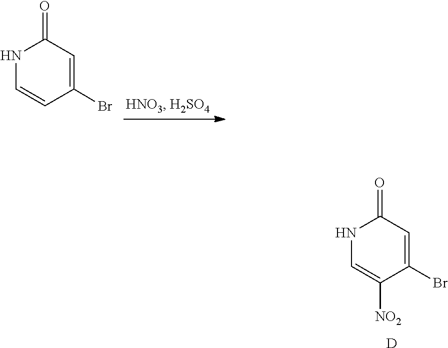

To a solution of 4-bromo-1H-pyridin-2-one (10.0 g, 57.5 mmol, CAS #36953-37-4) in H2SO₄ (80 mL) was added HNO3 (6.10 g, 96.8 mmol) dropwise at 0° C. for 2 hrs. Then the mixture was stirred at 70° C. for 15 hrs. On completion, the reaction mixture was quenched with ice water (500 mL) saturated NaOH aqueous solution (200 mL) was added to adjust the pH to 7. Then the mixture was extracted with EA (100 mL×3). The combined organic layers were washed with brinc (300 mL), dried over Na₂SO₄, filtered and concentrated in vacuo to give the title compound (5.10 g, 41% yield) as yellow solid. LC-MS (ESI+) m/z 221.0 (M+H)+.

### (3S)-1-(3-Pyridyl) piperidine-3-carbonyl chloride (Intermediate E)

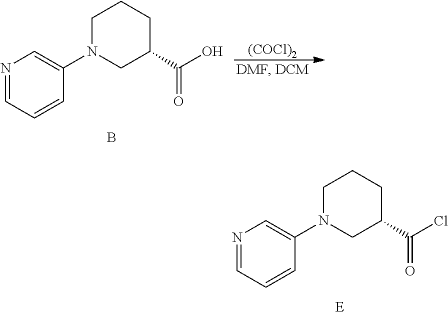

A solution of (3S)-1-(3-pyridyl) piperidine-3-carboxylic acid (50.0 mg, 242 umol, Intermediate B) and (COC1)₂ (46.1 mg, 363 umol) in DCM (0.5 mL) and DMF (0.01 mL) was stirred at 25° C. for 1 hr. On completion, the mixture was concentrated in vacuo to give the title compound (54.1 mg, 99% yield) as white liquid. LC-MS (ESI+) m/z 220.8 (M-Cl+MeOH)⁺.

### Ethyl 2-(5-amino-4-ethyl-2-oxo-1-pyridyl)acetate (Intermediate F)

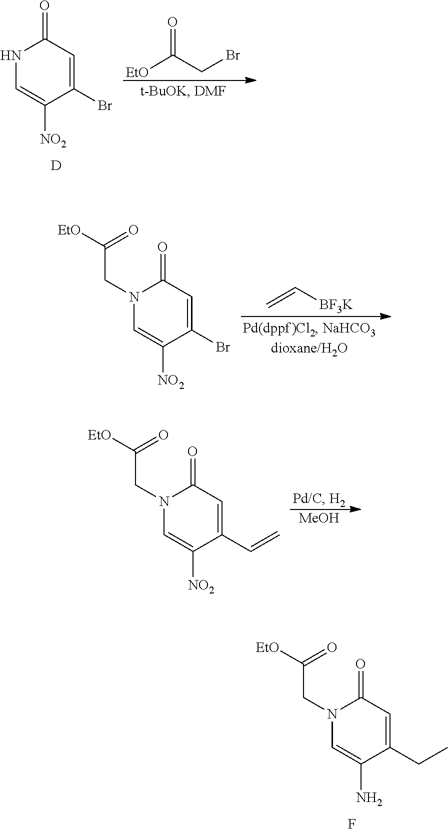

Step 1-Ethyl 2-(4-bromo-5-nitro-2-oxo-1-pyridyl)acetate. To a solution of 4-bromo-5-nitro-1H-pyridin-2-one (7.06 g, 32.2 mmol, Intermediate D) in DMF (140 mL) was added tBuOK (5.43 g, 48.3 mmol) at 0° C. and the mixture was stirred for 0.5 hrs. Then ethyl 2-bromoacetate (8.08 g, 48.3 mmol, CAS #105-36-2) was added into the mixture and the mixture was stirred at 25° C. for 2 hrs. On completion, the reaction mixture was diluted with EtOAc (600 mL) and washed water (100 mL×5). The organic layer was dried over Na₂SO₄, filtered and concentrated in vacuo. The residue was purified by column chromatography (SiO₂, PE: EA=5:1 to 3:1) to give the title compound (4.8 g) as yellow solid. 1H NMR (400 MHz, DMSO-d₆) δ 9.25 (s, 1H), 7.07 (s, 1H), 4.84 (s, 2H), 4.20-4.14 (m, 2H), 1.23-1.19 (m, 3H).

Step 2-Ethyl 2-(5-nitro-2-oxo-4-vinyl-1-pyridyl)acetate. A solution of ethyl 2-(4-bromo-5-nitro-2-oxo-1-pyridyl)acetate (500 mg, 1.64 mmol), potassium trifluoro (vinyl) boranuide (1.10 g, 8.19 mmol, CAS #13682-77-4), K₂CO₃ (679 mg, 4.92 mmol) and XPHOS-PD-G2 (128 mg, 163 umol) in dioxane (15 mL) was stirred at 80° C. for 3 hrs under N₂. On completion, the reaction mixture was diluted with EA (100 mL) and filtered. The mixture was washed with water (70 mL×3). The organic layer was dried over Na₂SO₄, filtered and concentrated in vacuo. The residue was purified by column chromatography (SiO₂, PE: EA=5:1) to give the title compound (240 mg, 58% yield) as brown oil. 1H NMR (400 MHZ, DMSO-d₆) δ 9.15 (s, 1H), 7.13-7.00 (m, 1H), 6.58 (s, 1H), 5.94-5.83 (m, 1H), 5.55 (d, J=11.6 Hz, 1H), 4.85 (s, 2H), 4.20-4.14 (m, 2H), 1.23-1.19 (m, 3H).

Step 3-Ethyl 2-(5-amino-4-ethyl-2-oxo-1-pyridyl)acetate. To a solution of ethyl 2-(5-nitro-2-oxo-4-vinyl-1-pyridyl)acetate (30 mg, 118 umol) and HCl (4 M, 0.2 mL) in MeOH (2 mL) was added Pd/C (30 mg, 28.1 umol, 10 wt %) under Ar₂ atmosphere. The suspension was degassed and purged with H₂ three times. The mixture was then stirred under H₂ (15 Psi) at 25° C. for 1.5 hrs. On completion, the reaction mixture was filtered and the filtrate was concentrated in vacuo to give the title compound (26.5 mg, 99% yield) as yellow solid. LC-MS (ESI+) m/z 225.3 (M+H)+.

### Tert-butyl N-(5-bromo-3-pyridyl)-N-methyl-carbamate (Intermediate G)

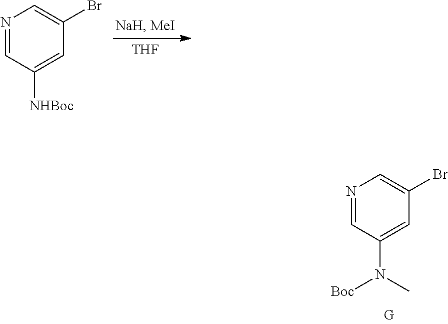 [SMILES: CC(C)(C)OC(=O)Nc1cncc(Br)c1 ; CN(C(=O)OC(C)(C)C)c1cncc(Br)c1 ; "THF"]

To a solution of tert-butyl N-(5-bromo-3-pyridyl) carbamate (5 g, 18.3 mmol, CAS #361550-43-8) in THF (30 mL) was added NaH (1.10 g, 27.4 mmol, 60% dispersion in mineral oil), and the mixture was stirred at 0° C. for 30 mins. Then Mel (3.12 g, 21.9 mmol) was added and the mixture was stirred at 25° C. for 2 hrs. On completion, the mixture was quenched with saturated NH₄Cl (3 mL) and diluted with H₂O (20 mL), then the mixture was stirred at 25° C. for 0.5 hr. The mixture was extracted with EA (10 mL×3). The combined organic layers were washed with saturated NaCl (10 mL), dried over anhydrous Na₂SO₄, filtered and concentrated in vacuo to give the residue. The residue was purified by flash silica gel chromatography (SiO₂, Petroleum ether/Ethyl acetate=4/1) to give the title compound (4 g, 76% yield) as pink solid. 1H NMR (400 MHZ, DMSO-d₆) δ 8.56 (d, J=2.4 Hz, 1H), 8.48 (d, J=2.0 Hz, 1H), 8.07 (t, J=2.0 Hz, 1H), 3.22 (s, 3H), 1.41 (s, 9H). LC-MS (ESI+) m/z 288.8 (M+H)+.

### (3S)-1-[5-[Tert-butoxycarbonyl (methyl)amino]-3-pyridyl] piperidine-3-carboxylic acid (Intermediate H)

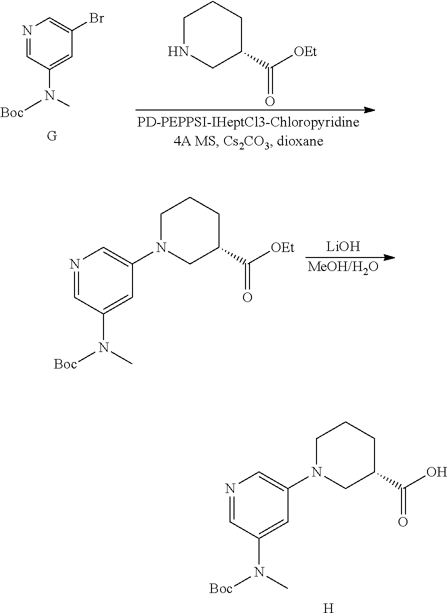

Step 1-Ethyl (3S)-1-[5-[tert-butoxycarbony (methyl)amino]-3-pyridyl] piperidine-3-carboxylate. To a solution of tert-butyl N-(5-bromo-3-pyridyl)-N-methyl-carbamate (1.7 g, 5.92 mmol, Intermediate G) and ethyl (3S)-piperidine-3-carboxylate (1.68 g, 10.6 mmol, CAS #37675-18-6) in dioxane (20 mL) was added PD-PEPPSI-IHeptCl 3-Chloropyridine (287 mg, 296 umol), Cs₂CO₃ (3.86 g, 11.8 mmol) and 4 Å molecular sieves (5.92 mmol). The mixture was stirred at 80° C. for 12 hrs. On completion, the reaction mixture was filtered and concentrated in vacuo to give a residue. Then the residue was purified by column chromatography (SiO₂, PE: EA=20:1 to PE: EA=5:1) to give the title compound (2.1 g, 97% yield) as yellow oil. 1H NMR (400 MHZ, DMSO-d₆) δ 8.08 (d, J=2.4 Hz, 1H), 7.92 (d, J=2.0 Hz, 1H), 7.22 (t, J=2.4 Hz, 1H), 4.08 (q, J=7.2 Hz, 2H), 3.66 (d, J=3.6, 12.4 Hz, 1H), 3.52-3.43 (m, 1H), 3.18 (s, 3H), 3.09 (d, J=9.4, 12.4 Hz, 1H), 2.96-2.86 (m, 1H), 2.68-2.58 (m, 1H), 1.98 (s, 1H), 1.94-1.85 (m, 1H), 1.75-1.60 (m, 2H), 1.39 (s, 9H), 1.18 (d, J=7.2 Hz, 3H). LC-MS (ESI+) m/z 364.6 (M+H)+.

Step 2-(3S)-1-[5-[Tert-butoxycarbonyl (methyl)amino]-3-pyridyl] piperidine-3-carboxylic acid. To a solution of ethyl (3S)-1-[5-[tert-butoxycarbonyl (methyl)amino]-3-pyridyl] piperidine-3-carboxylate (420 mg, 1.16 mmol) in MeOH (2 mL) and H₂O (0.4 mL) was added LiOH (83.0 mg, 3.47 mmol). The mixture was then stirred at 25° C. for 0.5 hr. On completion, the reaction mixture acidified with HCl (1 N) until the pH=3-4. Then the mixture was filtered to give the filter cake, which was dried in vacuo to give the title compound (300 mg, 77% yield) as a light yellow solid. 1H NMR (400 MHZ, DMSO-d₆) δ 8.08 (d, J=2.4 Hz, 1H), 7.91 (d, J=2.0 Hz, 1H), 7.22 (t, J=2.4 Hz, 1H), 3.69 (d, J=3.6, 12.4 Hz, 1H), 3.52 (d, J=12.4 Hz, 2H), 3.18 (s, 3H), 2.99 (d, J=9.6, 12.4 Hz, 1H), 2.90-2.80 (m, 1H), 1.97-1.84 (m, 1H), 1.76-1.66 (m, 1H), 1.64-1.51 (m, 2H), 1.46-1.31 (m, 9H). LC-MS (ESI+) m/z 336.0 (M+H)+.

### (3S)-1-(4-Isoquinolyl) piperidine-3-carbonyl chloride (Intermediate I)

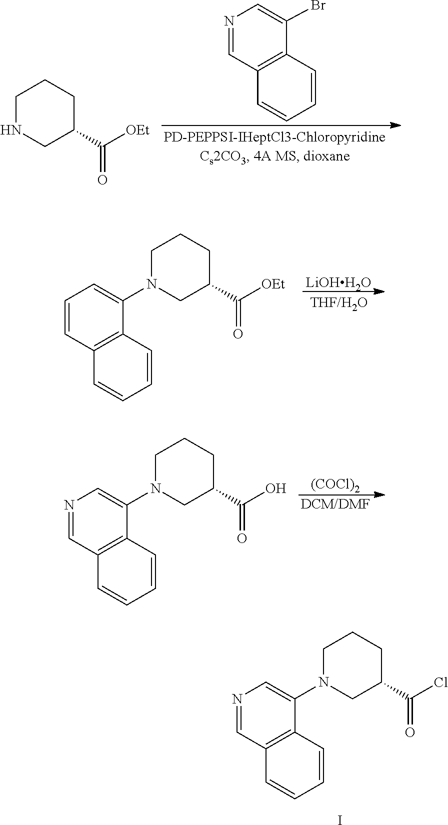

Step 1-Ethyl (3S)-1-(4-isoquinolyl) piperidine-3-carboxylate. A mixture of ethyl (3S)-piperidine-3-carboxylate (3 g, 19.0 mmol, CAS #37675-18-6), 4-bromoisoquinoline (4.76 g, 22.9 mmol, CAS #1532-97-4), Pd-PEPPSI-IHeptCl 3-Chloropyridine (1.85 g, 1.91 mmol), 4 Å molecular sieves (19.0 mmol) and Cs₂CO₃ (18.6 g, 57.2 mmol) in dioxane (50 mL) was stirred at 110° C. for 16 hrs. On completion, the reaction mixture was filtered and concentrated in vacuo to give the residue. The residue was diluted with H₂O (10 mL) and extracted with EA (20 mL×3). The combined organic layers were dried over Na₂SO₄, filtered and concentrated in vacuo to give the title compound (5.4 g, 99% yield) as yellow liquid. 1H NMR (400 MHZ, DMSO-d₆) δ 9.01 (s, 1H), 8.19 (s, 1H), 8.08-8.03 (m, 2H), 7.78-7.74 (m, 1H), 7.70-7.63 (m, 1H), 4.10 (q, J=7.2 Hz, 2H), 3.39 (d, J=9.6 Hz, 1H), 3.23-3.15 (m, 1H), 3.06 (t, J=9.6 Hz, 1H), 2.94-2.82 (m, 2H), 1.97-1.92 (m, 1H), 1.92-1.84 (m, 1H), 1.83-1.74 (m, 1H), 1.73-1.63 (m, 1H), 1.18 (t, J=7.2 Hz, 3H); LC-MS (ESI+) m/z 285.0 (M+H)+.

Step 2-(3S)-1-(4-Isoquinolyl) piperidine-3-carboxylic acid. To a solution of ethyl (3S)-1-(4-isoquinolyl) piperidine-3-carboxylate (1 g, 3.52 mmol) in THF (10 mL) and H₂O (2 mL) was added LiOH H₂O (590 mg, 14.0 mmol), then the mixture was stirred at 25° C. for 2 hrs. On completion, the reaction mixture was concentrated in vacuo to give the residue. Then the mixture was adjusted to pH=6 using HCl, diluted with H₂O (8 mL) and extracted with EA (15 mL×4). The combined organic layers were dried over Na₂SO₄, filtered and concentrated to give the title compound (0.9 g, 99% yield) as yellow solid. 1H NMR (400 MHZ, DMSO-d₆) δ 8.99 (s, 1H), 8.16 (s, 1H), 8.09 (t, J=8.4 Hz, 2H), 7.77-7.69 (m, 1H), 7.69-7.63 (m, 1H), 3.41 (d, J=10.0 Hz, 1H), 3.21 (d, J=11.2 Hz, 1H), 2.97 (t, J=10.0 Hz, 1H), 2.85 (t, J=9.6 Hz, 1H), 2.73-2.65 (m, 1H), 1.99 (t, J=6.4 Hz, 1H), 1.88-1.83 (m, 1H), 1.82-1.82 (m, 1H), 1.77-1.68 (m, 1H), 1.67-1.54 (m, 1H); LC-MS (ESI+) m/z 257.0 (M+H)+.

Step 3-(3S)-1-(4-isoquinolyl) piperidine-3-carbonyl chloride. To a solution of (3S)-1-(4-isoquinolyl) piperidine-3-carboxylic acid (200 mg, 780 umol) and DMF (2.85 mg, 39.0 umol) in DCM (3 mL) was added (COCI)₂ (198 mg, 1.56 mmol) dropwise at 0° C. Then the mixture was stirred at 25° C. for 0.5 hr. On completion, the reaction mixture was concentrated in vacuo to give the title compound (210 mg, 97% yield) as white solid.

### 3-(5-Methoxy-3-pyridyl)benzoyl chloride (Intermediate J)

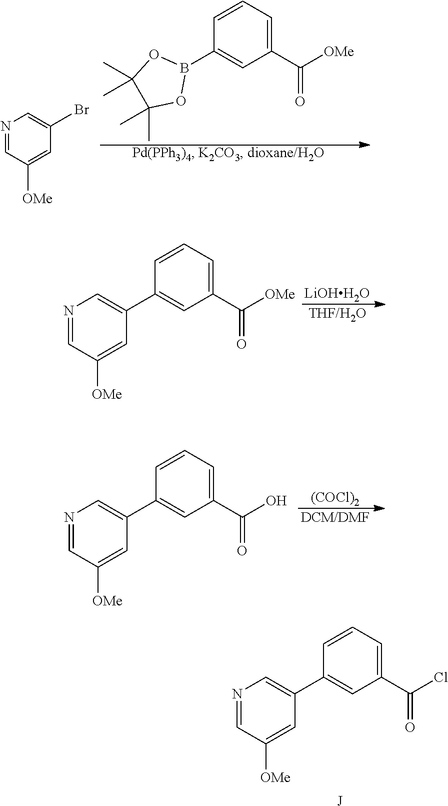

Step 1-Methyl 3-(5-methoxy-3-pyridyl)benzoate. To a solution of 3-bromo-5-methoxy-pyridine (3 g, 15.9 mmol, CAS #50720 Dec. 2) and methyl 3-(4,4,5,5-tetramethyl-1,3,2-dioxaborolan-2-yl)benzoate (5.02 g, 19.1 mmol, CAS #480425-35-2) in a mixture of dioxane (40 mL) and H₂O (10 mL) was added Pd (PPh3)₄ (921 mg, 797 umol) and K₂CO₃ (6.62 g, 47.8 mmol). Then the reaction mixture was stirred at 100° C. for 16 hrs. On completion, the reaction mixture was filtered and concentrated to give the residue, which was then diluted with H₂O (100 mL) and extracted with EA (100 mL×3). The combined organic layers were dried over Na₂SO₄, filtered and concentrated to give the title compound (3.5 g, 90% yield) as brown solid. 1H NMR (400 MHZ, DMSO-d₆) δ 8.49 (d, J=1.2 Hz, 1H), 8.33 (d, J=2.4 Hz, 1H), 8.23 (s, 1H), 8.04-7.98 (m, 2H), 7.68-7.63 (m, 2H), 3.91 (d, J=10.8 Hz, 6H); LC-MS (ESI+) m/z 244.2 (M+H)+.

Step 2-3-(5-Methoxy-3-pyridyl)benzoic acid. To a solution of methyl 3-(5-methoxy-3-pyridyl)benzoate (1.00 g, 4.11 mmol) in THF (10 mL) and H₂O (2 mL) was added LiOH.H₂O (689 mg, 16.4 mmol), then the mixture was stirred at 25° C. for 2 hrs. On completion, the reaction mixture was adjusted to pH=6 using AcOH. A solid precipitated which was then filtered and the filter cake was dried. The crude solid was triturated with EA (10 mL) to give the title compound (495 mg, 52% yield) as white solid. ¹H NMR (400 MHZ, DMSO-d₆) δ 8.47 (d, J=1.6 Hz, 1H), 8.29 (d, J=2.8 Hz, 1H), 8.20 (s, 1H), 7.94 (d, J=7.6 Hz, 1H), 7.73 (d, J=8.0 Hz, 1H), 7.61-7.58 (m, 1H), 7.45 (t, J=7.6 Hz, 1H), 1.88 (s, 3H); LC-MS (ESI+) m/z 230.0 (M+H)+.

Step 3-3-(5-Methoxy-3-pyridyl)benzoyl chloride. To a solution of 3-(5-methoxy-3-pyridyl)benzoic acid (150 mg, 654 umol) and DMF (2.39 mg, 32.7 umol) in DCM (3 mL) was added (COC1)₂ (166 mg, 1.31 mmol) dropwise at 0° C. Then the mixture was stirred at 25° C. for 0.5 hr. On completion, the reaction mixture was concentrated in vacuo to give the title compound (150 mg, 92% yield) as white solid.

### (3S)-1-(5-Methoxy-3-pyridyl) piperidine-3-carbonyl chloride (Intermediate K)

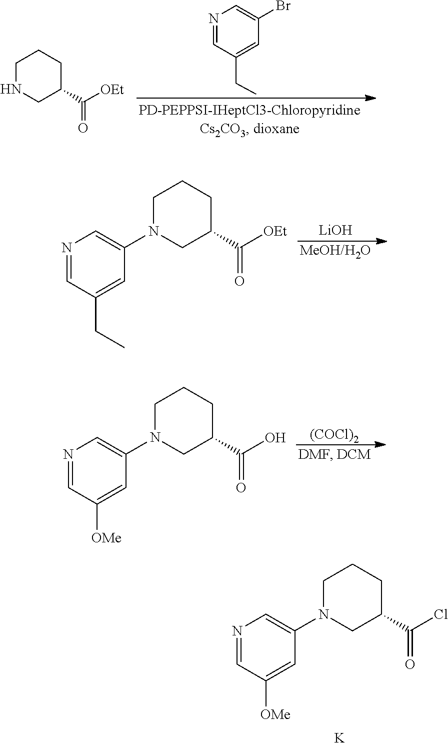

Step 1-Ethyl (3S)-1-(5-methoxy-3-pyridyl) piperidine-3-carboxylate. A mixture of 3-bromo-5-methoxy-pyridine (800 mg, 4.25 mmol, CAS #37675-18-6), ethyl (3S)-piperidine-3-carboxylate (668 mg, 4.25 mmol, CAS #50720 Dec. 2), PD-PEPPSI-IHeptCl 3-Chloropyridine (206 mg, 212 umol) and Cs₂CO₃ (2.77 g, 8.51 mmol) in dioxane (15 mL) was stirred at 90° C. for 16 hrs under N₂. On completion, the reaction was diluted with EA (100 mL). The organic layer was washed with water (70 mL×3), dried over Na₂SO₄ and filtered. The filtrate was concentrated in vacuo. The residue was purified by column chromatography (SiO₂, Petroleum ether/Ethyl acetate=3/1 to 1/1) to give the title compound (800 mg, 71% yield) as yellow oil. 1H NMR (400 MHZ, CDC13)δ 7.97 (d, J=2.4 Hz, 1H), 7.80 (d, J=2.4 Hz, 1H), 6.74 (t, J=2.4 Hz, 1H), 4.18 (q, J=7.2 Hz, 2H), 3.85 (s, 3H), 3.73-3.65 (m, 1H), 3.52-3.44 (m, 1H), 3.12 (dd, J=9.6, 12.4 Hz, 1H), 2.95-2.86 (m, 1H), 2.70-2.61 (m, 1H), 2.09-2.00 (m, 1H), 1.87-1.79 (m, 1H), 1.75-1.64 (m, 2H), 1.28 (t, J=7.2 Hz, 3H).

Step 2-(3S)-1-(5-methoxy-3-pyridyl) piperidine-3-carboxylic acid. To a solution of ethyl (3S)-1-(5-methoxy-3-pyridyl) piperidine-3-carboxylate (700 mg, 2.65 mmol) in MeOH (7 mL) and H₂O (2 mL) was added LiOH.H₂O (555 mg, 13.2 mmol). Then the reaction was stirred at 25° C. for 1 hr. On completion, the reaction was acidized with HCl (4 N) until the pH<6. The reaction then diluted with water (50 mL) and the mixture was lyophilized to give the title compound (620 mg, 99% yield) as yellow solid. LC-MS (ESI+) m/z 237.1 (M+H)+.

Step 3-(3S)-1-(5-methoxy-3-pyridyl) piperidine-3-carbonyl chloride. To a solution of (3S)-1-(5-methoxy-3-pyridyl) piperidine-3-carboxylic acid (150 mg, 634 umol) in DCM (2 mL) was added (COC1)₂ (120 mg, 952 umol) and DMF (464 ug, 6.35 umol), then the reaction was stirred at 25° C. for 1 hr. On completion, the reaction mixture was concentrated in vacuo to give the title compound (160 mg, 98% yield) as yellow liquid. LC-MS (ESI+) m/z 250.9 (M-C1+MeOH)+.

### (3S)-1-(5-Ethyl-3-pyridyl) piperidine-3-carboxylic acid (Intermediate L)

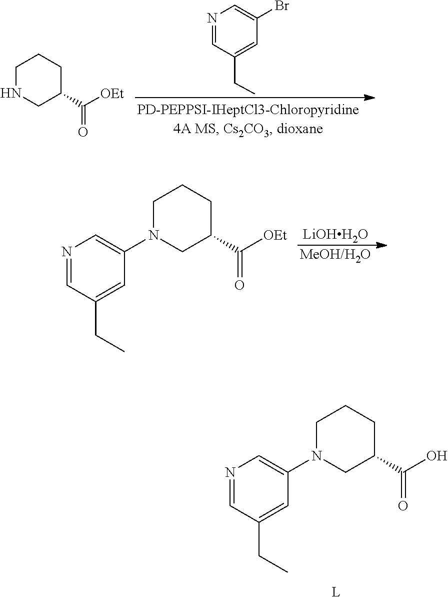

Step 1-Ethyl (3S)-1-(5-ethyl-3-pyridyl) piperidine-3-carboxylate. A mixture of ethyl (3S)-piperidine-3-carboxylate (2.00 g, 12.7 mmol, CAS #37675-18-6), 3-bromo-5-ethyl-pyridine (2.37 g, 12.72 mmol, CAS #142337-95-9), Cs₂CO₃ (12.4 g, 38.2 mmol) and 4 Å molecular sieves (200 mg) in dioxane (50 mL) was added 1,3-bis [2,6-bis(1-propylbutyl)phenyl]-4,5-dichloro-2H-imidazol-1-ium-2-ide 3-chloropyridine;dichloropalladium (371 mg, 382 umol). Then the mixture was degassed and purged with N₂ three times. Next, the mixture was stirred at 90° C. for 15 hrs under N₂ atmosphere. On completion, the reaction mixture was concentrated in vacuo to give a residue. Then the residue was diluted with H₂O (50 mL) and extracted with EA (100 mL×3). The combined organic layers were washed with saturated NaCl with H₂O (50 mL), dried over Na₂SO₄, filtered and concentrated in vacuo to give a residue. The residue was purified by column chromatography (SiO₂, PE: EA=1:0 to 0:1) to give the title compound (3.20 g, 96% yield) as yellow liquid. 1H NMR (400 MHZ, DMSO-d₆) δ 8.10 (d, J=2.4 Hz, 1H), 7.86 (s, 1H), 7.15 (s, 1H), 4.09 (q, J=7.2 Hz, 2H), 3.65-3.55 (m, 1H), 3.50-3.42 (m, 1H), 3.09-3.00 (m, 1H), 2.92-2.84 (m, 1H), 2.66-2.59 (m, 1H), 2.55 (q, J=7.6 Hz, 2H), 1.94-1.86 (m, 1H), 1.75-1.67 (m, 1H), 1.66-1.52 (m, 2H), 1.23-1.14 (m, 6H); LC-MS (ESI+) m/z 262.9 (M+H)+.

Step 2-(3S)-1-(5-ethyl-3-pyridyl) piperidine-3-carboxylic acid. A mixture of ethyl (3S)-1-(5-ethyl-3-pyridyl) piperidine-3-carboxylate (1.00 g, 3.81 mmol) in H₂O (1 mL) and MeOH (5 mL) was added LiOH.H₂O (640 mg, 15.3 mmol), and the mixture was stirred at 25° C. for 1 hr. On completion, the reaction mixture was concentrated in vacuo to give a residue. The residue was diluted with ACN (5 mL) and HCl (4 M) was added to adjust the pH to 5, then the mixture was concentrated in vacuo to give a residue. The residue was purified by column chromatography (SiO₂, DCM: EtOH=10:1) to give the title compound (350 mg, 39% yield, 92% ee) as red liquid. 1H NMR (400 MHZ, DMSO-d₆) δ 12.69-11.96 (m, 1H), 8.11 (s, 1H), 7.86 (s, 1H), 7.17 (s, 1H), 3.67-3.59 (m, 1H), 3.50 (d, J=12.0 Hz, 1H), 2.99-2.90 (m, 1H), 2.89-2.80 (m, 1H), 2.59-2.52 (m, 3H), 1.96-1.84 (m, 1H), 1.77-1.67 (m, 1H), 1.65-1.49 (m, 2H), 1.17 (t, J=7.6 Hz, 3H); LC-MS (ESI+) m/z 235.0 (M+H).

### (3S)-1-(8-Methoxy-4-isoquinolyl) piperidine-3-carboxylic acid (Intermediate M)

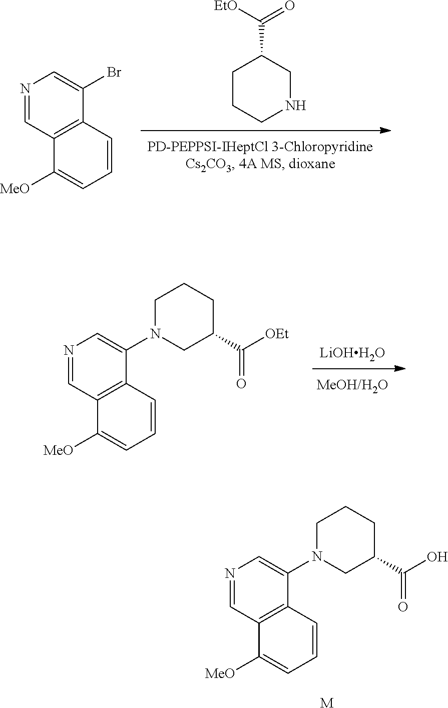

Step 1-Ethyl (3S)-1-(8-methoxy-4-isoquinolyl) piperidine-3-carboxylate. A mixture of 4-bromo-8-methoxy-isoquinoline (450 mg, 1.89 mmol, CAS #1784377-21-4), ethyl (3S)-piperidine-3-carboxylate (535 mg, 3.40 mmol, CAS #37675-18-6), PD-PEPPSI-IHeptCl 3-Chloropyridine (184 mg, 189 umol), Cs₂CO₃ (1.23 g, 3.78 mmol) and 4 Å molecular sieves (500 mg) in dioxane (8 mL). Then the mixture was purged with N₂ three times and the mixture was stirred at 100° C. for 24 hrs. On completion, the reaction was filtered and concentrated in vacuo to give the residue. The residue was purified by column chromatography (SiO₂, PE/EA=10/1 to 6/1) to give the title compound (800 mg, 67% yield) as yellow oil. 1H NMR (400 MHZ, DMSO-d₆) δ 9.20 (s, 1H), 8.21 (s, 1H), 7.74-7.64 (m, 1H), 7.63-7.56 (m, 1H), 7.10 (d, J=7.6 Hz, 1H), 4.10 (q, J=7.2 Hz, 2H), 4.00 (s, 3H), 3.38 (d, J=9.6 Hz, 1H), 3.24-3.12 (m, 1H), 3.10-2.96 (m, 1H), 2.94-2.79 (m, 2H), 2.03-1.92 (m, 1H), 1.91-1.83 (m, 1H), 1.82-1.61 (m, 2H), 1.18 (t, J=7.2 Hz, 3H); LC-MS (ESI+) m/z 315.2 (M+H)+.

Step 2-(3S)-1-(8-methoxy-4-isoquinolyl) piperidine-3-carboxylic acid. To a solution of ethyl (3S)-1-(8-methoxy-4-isoquinolyl) piperidine-3-carboxylate (760 mg, 2.42 mmol) in MeOH (7.6 mL) and H₂O (1.9 mL) was added LiOH.H₂O (304 mg, 7.25 mmol), then the mixture was stirred at 20° C. for 2 hrs. On completion, the reaction was concentrated in vacuo to give a residue. The residue was purified by prep-HPLC (column: Phenomenex luna C18 150*25 mm*10 um; mobile phase: [water (FA)-ACN]) to give the title compound (600 mg, 86% yield, FA) as yellow solid. 1H NMR (400 MHZ, DMSO-d₆) δ 12.38 (s, 1H), 9.20 (s, 1H), 8.20 (s, 1H), 7.72-7.65 (m, 1H), 7.65-7.58 (m, 1H), 7.10 (d, J=7.6 Hz, 1H), 4.00 (s, 3H), 3.40 (d, J=9.6 Hz, 1H), 3.19 (d, J=11.6 Hz, 1H), 2.98 (t, J=10.0 Hz, 1H), 2.85 (t, J=9.6 Hz, 1H), 2.80-2.72 (m, 1H), 2.04-1.94 (m, 1H), 1.92-1.83 (m, 1H), 1.82-1.70 (m, 1H), 1.69-1.55 (m, 1H); LC-MS (ESI+) m/z 287.0 (M+H)+.

### (5-{N-[8-(1,3-Dioxoisoindol-2-yl) octyl] piperidine-3-amido}-2-oxopyridin-1-yl) acetic acid (Intermediate N)

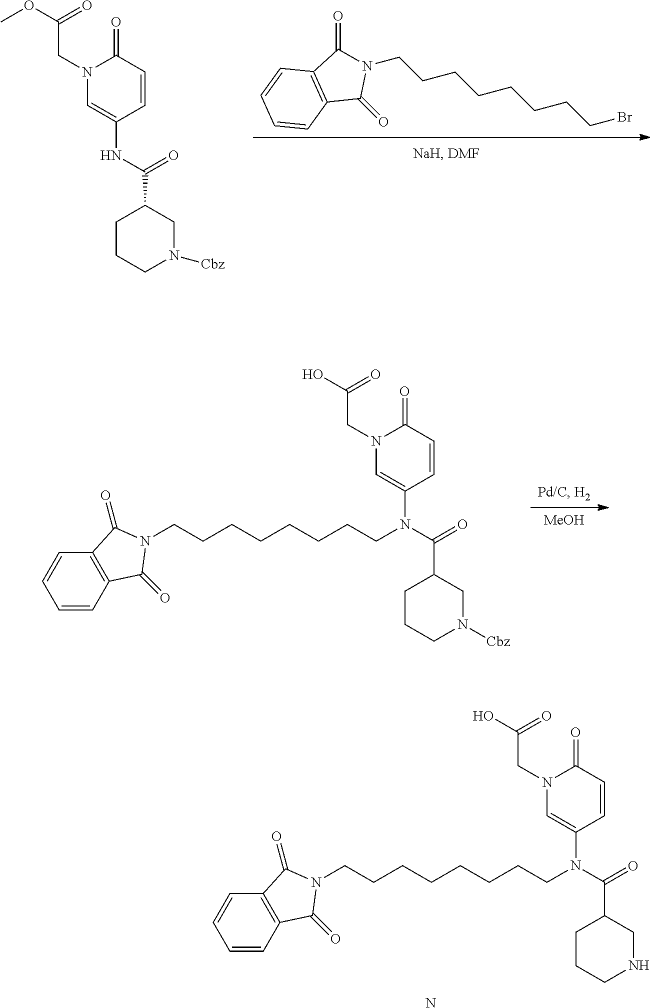

Step 1-(5-{N-[8-(1,3-dioxoisoindol-2-yl) octyl] 1-[(benzyloxy) carbonyl]piperidine-3-amido}-2-oxopyridin-1-yl) acetic acid. To a stirred mixture of benzyl (3S)-3-{[1-(2-methoxy-2-oxoethyl)-6-oxopyridin-3-yl]carbamoyl} piperidine-1-carboxylate (3.1 g, 7.2 mmol, Intermediate AU) in DMF (31 mL) was added NaH (870 mg, 21.7 mmol, 60% dispersion in mineral oil) at 0° C. The resulting mixture was stirred for 40 min at rt. Next, 2-(8-bromooctyl) isoindole-1,3-dione (3.68 g, 10.9 mmol, CAS #17702-83-9) was added at 0° C. to the reaction mixture. The reaction was then stirred for 1 hr at 50° C. On completion, the reaction was quenched with sat. NH₄Cl (aq.)(50 mL) at 0° C. The resulting mixture was extracted with EtOAc (3×100 mL). The combined organic layers were washed with brine (3×100 mL), dried over anhydrous Na₂SO₄. After filtration, the filtrate was concentrated under reduced pressure. The resulting mixture was concentrated under vacuum. The residue was then dissolved in MeOH (20 mL) and the solution was purified by reverse phase Flash chromatography (Column: WelFlash™ C18-I, 20-40 um, 330 g; Eluent A: Water (plus or 10 mmol/L NH₄HCO3); Eluent B: ACN; Gradient: 25%-50% B in 35 min; Flow rate: 80 mL/min; Detector: 220/254 nm; desired fractions were collected at 34% B) and concentrated under reduced pressure to afford the title compound (1.1 g, 23% yield) as a dark yellow solid. LC/MS (ESI, m/z): [(M+1)]+=671.4; 1H NMR (400 MHZ, Chloroform-d)δ 7.84 (dd, J=5.4, 3.1 Hz, 2H), 7.71 (dd, J=5.5, 3.0 Hz, 2H), 7.38-7.29 (m, 6H), 7.20-7.07 (m, 1H), 6.64-6.50 (m, 1H), 4.59-4.34 (m, 2H), 4.31-4.17 (m, 1H), 4.14-3.94 (m, 2H), 3.66 (t, J=7.3 Hz, 2H), 3.57-3.39 (m, 2H), 3.06-2.87 (m, 1H), 2.81-2.63 (m, 2H), 2.5-2.38 (m, 1H), 1.80-1.63 (m, 4H), 1.62-1.41 (m, 4H), 1.37-1.15 (m, 8H).

Step 2-(5-{N-[8-(1,3-dioxoisoindol-2-yl) octyl] piperidine-3-amido}-2-oxopyridin-1-yl) acetic acid. To a stirred mixture of (5-{N-[8-(1,3-dioxoisoindol-2-yl) octyl] 1-[(benzyloxy) carbonyl]piperidine-3-amido}-2-oxopyridin-1-yl) acetic acid (1.1 g, 1.6 mmol) in MeOH (25 mL) was added Pd/C (0.52 g, 4.92 mmol) at rt under nitrogen atmosphere. The resulting mixture was stirred for 16 h at rt under nitrogen atmosphere. On completion, the resulting mixture was filtered, and the filter cake was washed with MeOH (3×30 mL). The filtrate was then concentrated under reduced pressure to afford the title compound (670 mg, 76% yield) as an off-white solid. LC/MS (ESI, m/z): [(M+1)]+=537.4; 1H NMR (300 MHz, DMSO-d₆)δ 7.92-7.80 (m, 4H), 7.74-7.67 (m, 1H), 7.41-7.27 (m, 1H), 6.42-6.36 (m, 1H), 4.66-4.41 (m, 2H), 3.55 (t, J=7.1 Hz, 2H), 3.40-3.20 (m, 3H), 3.13-3.02 (m, 1H), 2.99-2.85 (m, 1H), 2.83-2.69 (m, 2H), 1.69-1.50 (m, 4H), 1.45-1.32 (m, 4H), 1.29-1.09 (m, 8H).

{5-[N-(8-Aminooctyl)₁-(isoquinolin-4-yl) piperidine-3-amido]-2-oxopyridin-1-yl} acetic acid (Intermediate O)

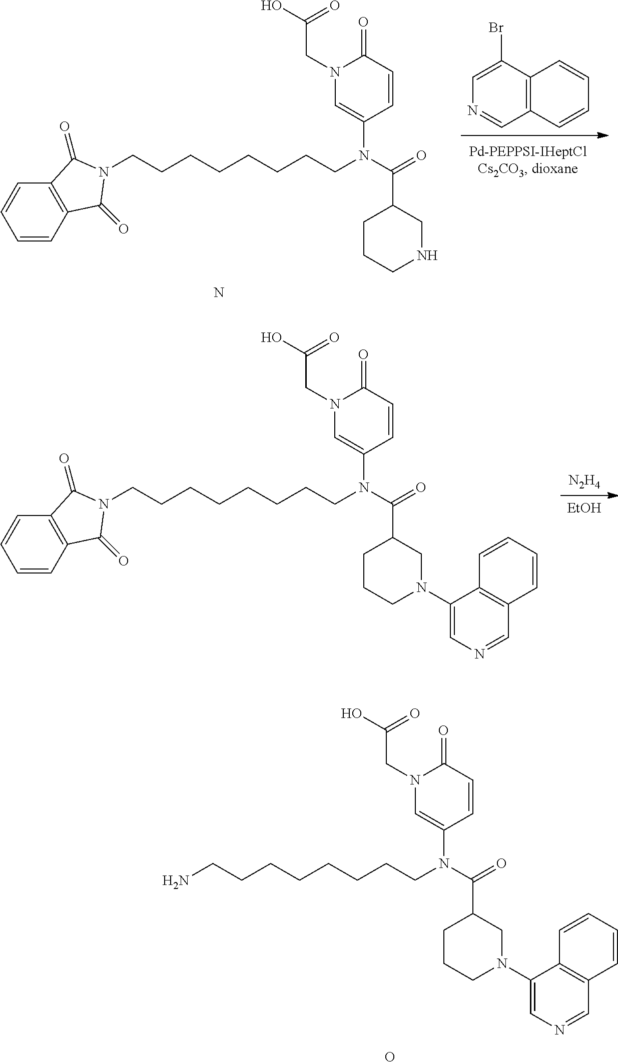

Step 1-(5-{N-[8-(1,3-dioxoisoindol-2-yl) octyl] 1-(isoquinolin-4-yl) piperidine-3-amido}-2-oxopyridin-1-yl) acetic acid. To a stirred mixture of (5-{N-[8-(1,3-dioxoisoindol-2-yl) octyl] piperidine-3-amido}-2-oxopyridin-1-yl) acetic acid (670 mg, 1.25 mmol, Intermediate N) and 4-bromoisoquinoline (311.72 mg, 1.499 mmol) in 1,4-dioxane (10 mL) were added Pd-PEPPSI-IHeptCl 3-chloropyridine (182.22 mg, 0.187 mmol) and Cs₂CO₃ (813.59 mg, 2.498 mmol) at rt under nitrogen atmosphere. The resulting mixture was stirred for 16 h at 80° C. under nitrogen atmosphere. On completion, the mixture was cooled to rt and diluted with water (50 mL). The mixture was extracted with EtOAc (3×20 mL). The combined organic layers were washed with brine (3×10 mL), and dried over anhydrous Na₂SO₄. After filtration, the filtrate was concentrated under reduced pressure. The residue was purified by reverse phase flash chromatography (Column: WelFlash™ C18-I, 20-40 um, 330 g; Eluent A: Water (plus 10 mmol/L NH₄HCO3); Eluent B: ACN; Gradient: 30%-60% B in 45 min; Flow rate: 80 mL/min; Detector: 220/254 nm; desired fractions were collected at 36% B) and concentrated under reduced pressure to afford the title compound (128 mg, 16% yield) as a yellow oil. LC/MS (ESI, m/z): [(M+1)]=664.3.

Step 2-{5-[N-(8-aminooctyl)₁-(isoquinolin-4-yl) piperidine-3-amido]-2-oxopyridin-1-yl} acetic acid. To a stirred solution of (5-{N-[8-(1,3-dioxoisoindol-2-yl) octyl] 1-(isoquinolin-4-yl) piperidine-3-amido}-2-oxopyridin-1-yl) acetic acid (128 mg, 0.193 mmol) in EtOH (2 mL) was added hydrazine hydrate (0.4 mL, 98% solution) at rt. The resulting mixture was stirred for 30 min at 70° C. On completion, the reaction mixture was concentrated under vacuum. The residue was purified by reverse phase flash chromatography (Column: WelFlash™ C18-I, 20-40 um, 80 g; Eluent A: Water (plus 10 mmol/L TFA); Eluent B: ACN; Gradient: 5%-30% B in 30 min; Flow rate: 60 mL/min; Detector: 220/254 nm; desired fractions were collected at 21% B) and concentrated under reduced pressure to afford the title compound (51 mg, 50% yield) as a yellow oil. LC/MS (ESI, m/z): [(M+1)]+=534.3; 1H NMR (400 MHZ, DMSO-d₆) § 9.39 (s, 1H), 8.39 (d, J=8.2 Hz, 1H), 8.20 (s, 1H), 8.02-7.89 (m, 4H), 7.71-7.61 (m, 2H), 7.56-7.48 (m, 1H), 6.53 (d, J=9.6 Hz, 1H), 4.83-4.46 (m, 3H), 3.38 (d, J=10.1 Hz, 2H), 2.98-2.82 (m, 4H), 2.81-2.70 (m, 2H), 1.93-1.54 (m, 4H), 1.54-1.34 (m, 4H), 1.31-1.17 (m, 8H).

### 2,3,4,5,6-Pentafluorophenyl 2-[(9S)-7-(4-chlorophenyl)-4,5,13-trimethyl-3-thia-1,8,11,12-tetraazatricyclo [8.3.0.0∧{2,6}] trideca-2 (6),4,7,10,12-pentaen-9-yl] acetate (Intermediate P)

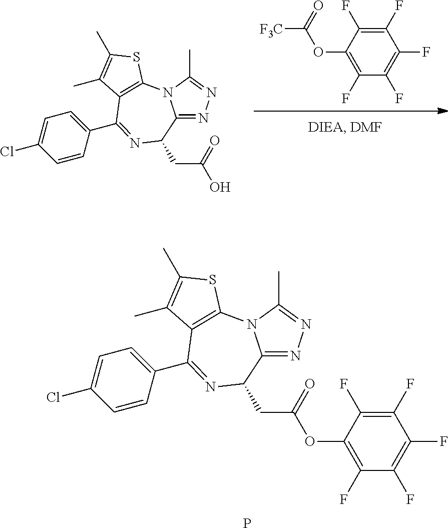

To a solution of [(9S)-7-(4-chlorophenyl)-4,5,13-trimethyl-3-thia-1,8,11,12-tetraazatricyclo [8.3.0.0∧{2,6}] trideca-2 (6),4,7,10,12-pentaen-9-yl]acetic acid (2.0 g, 5.0 mmol, CAS #202592-23-2) and 2,3,4,5,6-pentafluorophenyl 2,2,2-trifluoroacetate (2.8 g, 10 mmol, CAS #14533-84-7) in DMF (10 mL) was added DIEA (4.5 g, 34.817 mmol). The resulting mixture was stirred for 3 h at rt. On completion, the mixture was concentrated under reduced pressure. The residue was purified by silica gel column chromatography, eluted with petroleum ether/EtOAc (1:1), to afford the title compound (1.9 g, 67% yield) as a yellow oil. LC/MS (ESI, m/z): [(M+1)]+=567.2; 1H NMR (400 MHZ, DMSO-d₆) δ 7.52 (d, J=8.7 Hz, 2H), 7.45 (d, J=8.5 Hz, 2H), 4.68 (dd, J=8.5, 5.9 Hz, 1H), 3.97-3.78 (m, 2H), 2.63 (s, 3H), 2.42 (s, 3H), 1.63 (s, 3H).

### 2-(5-(N-(2-(2-(2-(2-Azidoethoxy) ethoxy) ethoxy)ethyl) piperidine-3-carboxamido)-2-oxopyridin-1 (2H)-yl) acetic acid (Intermediate Q)

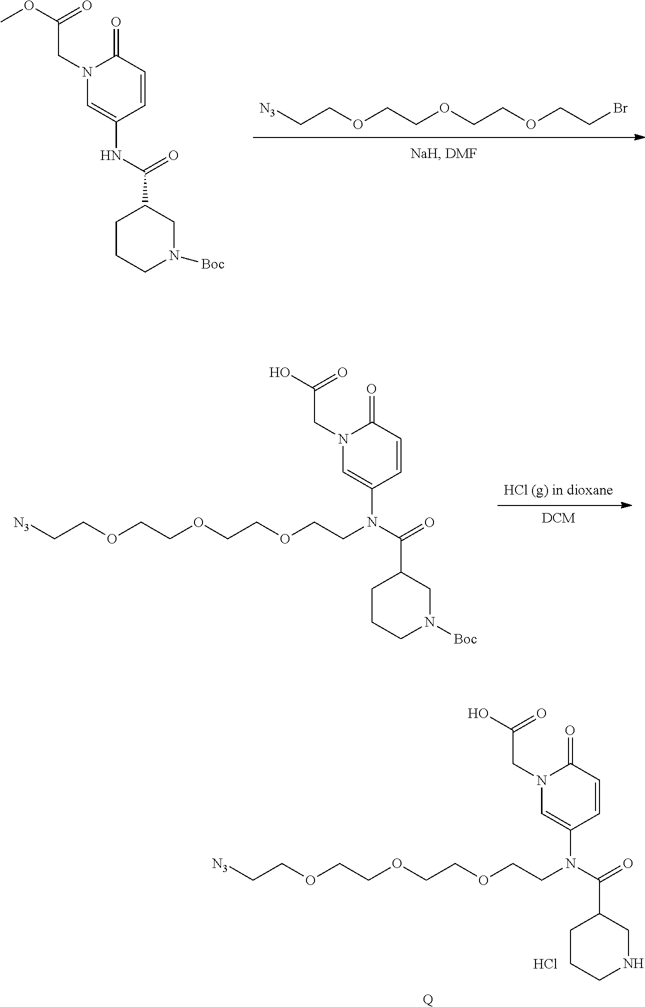

Step 1 2-(5-(N-(2-(2-(2-(2-azidoethoxy) ethoxy) ethoxy)ethyl)-1-(tert-butoxycarbonyl) piperidine-3-carboxamido)-2-oxopyridin-1 (2H)-yl) acetic acid. To a stirred mixture of tert-butyl(S)-3-((1-(2-methoxy-2-oxoethyl)-6-oxo-1,6-dihydropyridin-3-yl) carbamoyl) piperidine-1-carboxylate (2.9 g, 7.4 mmol, Intermediate AV) in DMF (50 mL) was added NaH (0.88 g, 22 mmol, 60% dispersion in mineral oil) in portions at 0° C. under nitrogen atmosphere. The resulting mixture was stirred for 30 min at 0° C. under nitrogen atmosphere. Next, a solution of 1-azido-2-{2-[2-(2-bromoethoxy)ethoxy]ethoxy} ethane (4.16 g, 14.7 mmol, CAS #1446282-43-4) in DMF (15 mL) was added to the reaction mixture at rt. The resulting mixture was stirred for an additional 16 h at 55° C. On completion, the reaction mixture was diluted with water (800 mL) and extracted with EtOAc (3×300 mL). The combined organic layer was dried over anhydrous Na₂SO₄. After filtration, the filtrate was concentrated under reduced pressure. The residue was purified by silica gel column chromatography, eluted with CH₂Cl2/MeOH (15:1), to afford the title compound (which racemized)(1.4 g, 33% yield) as an yellow oil. 1H NMR (300 MHz, Chloroform-d) δ 7.61-7.54 (m, 1H), 7.29-7.21 (m, 1H), 6.62-6.51 (t, J=9.6, 4.8 Hz, 1H), 4.56 (s, 2H), 4.05-3.96 (m, 2H), 3.75-3.61 (m, 14l H), 3.59-3.52 (m, 1H), 3.35-3.21 (m, 1H), 2.92-2.85 (m, 1H), 2.78-2.69 (m 1H), 2.48-2.39 (m, 1H), 1.82-1.71 (m 1H), 1.69-1.57 (m, 1H), 1.52-1.46 (m, 2H), 1.42 (s, 9H); LC/MS (ESI, m/z): [(M+H)]+=581.4.

Step 2-2-(5-(N-(2-(2-(2-(2-azidoethoxy) ethoxy) ethoxy)ethyl) piperidine-3-carboxamido)-2-oxopyridin-1 (2H)-yl) acetic acid hydrochloride. To a stirred solution of 2-(5-(N-(2-(2-(2-(2-azidoethoxy) ethoxy) ethoxy)ethyl)-1-(tert-butoxycarbonyl) piperidine-3-carboxamido)-2-oxopyridin-1 (2H)-yl) acetic acid (1.3 g, 2.2 mmol) in DCM (30 mL) was added a solution of 4 M HCl (gas) in 1,4-dioxane (15 mL, 60 mmol) dropwise at 0° C. under air atmosphere. The resulting mixture was stirred for 1 h at rt under nitrogen atmosphere. On completion, the reaction mixture was concentrated under reduced pressure to afford the title compound (1.1 g, 95% yield) as an yellow oil. LC/MS (ESI, m/z): [(M+H)]+=481.4.

### 2-(5-(N-(2-(2-(2-(2-Aminoethoxy) ethoxy) ethoxy)ethyl)-1-(isoquinolin-4-yl) piperidine-3-carboxamido)-2-oxopyridin-1 (2H)-yl) acetic acid (Intermediate R)

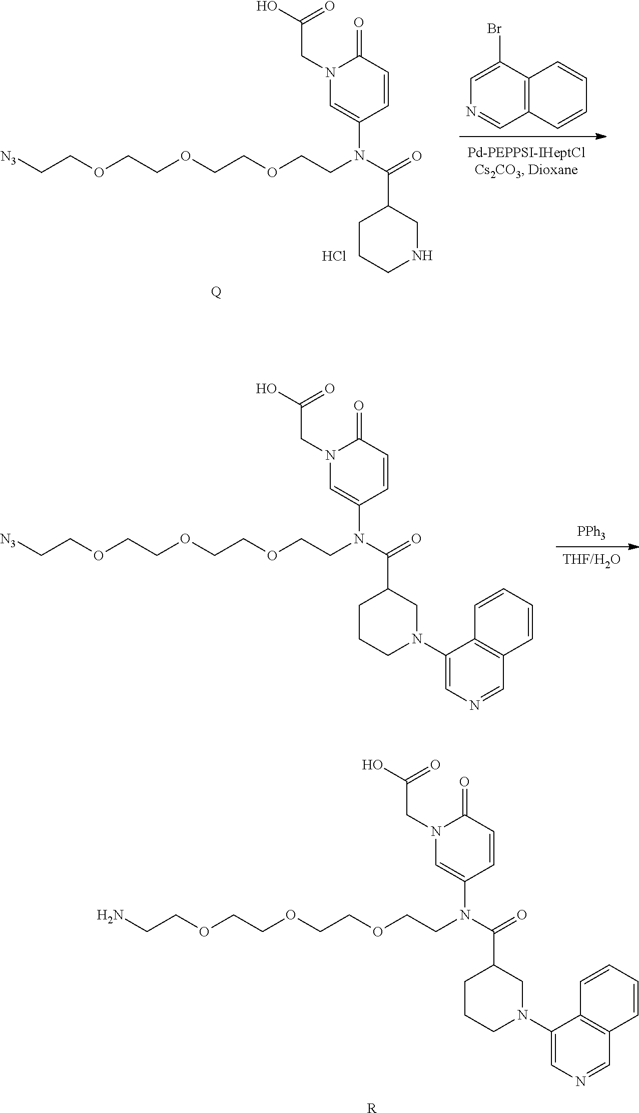

Step 1-2-(5-(N-(2-(2-(2-(2-azidoethoxy) ethoxy) ethoxy)ethyl)-1-(isoquinolin-4-yl) piperidine-3-carboxamido)-2-oxopyridin-1 (2H)-yl) acetic acid. To a stirred mixture of 2-(5-(N-(2-(2-(2-(2-azidoethoxy) ethoxy) ethoxy)ethyl) piperidine-3-carboxamido)-2-oxopyridin-1 (2H)-yl) acetic acid hydrochloride (1.0 g, 2.0 mmol, Intermediate Q) and Cs₂CO₃ (2.03 g, 6.24 mmol) in 1,4-dioxane (30 mL) were added 4-bromoisoquinoline (0.43 g, 2.08 mmol, CAS #1532-97-4) and Pd-PEPPSI-IHeptCl 3-chloropyridine (0.10 g, 0.10 mmol) at rt under nitrogen atmosphere. The resulting mixture was stirred for 16 h at 80° C. under nitrogen atmosphere. On completion, the mixture was cooled to rt and concentrated under reduced pressure. The residue was purified by reverse flash chromatography (column: WelFlash™ C18-I,20-40 um,330g; Eluent A: Water (plus 10 mmol/L FA);Eluent B: ACN, 15% to 35% gradient in 25 min; Flow rate: 80 mL/min; detector: UV 220/254 nm, desired fractions were collected at 23% B) and concentrated under reduced pressure to afford the title compound (680 mg, 50% yield) as an yellow oil. 1H NMR (400 MHZ, Chloroform-d) δ 9.09-9.01 (m, 1H), 8.12-8.05 (m, 2H), 7.94-7.82 (m, 1H), 7.73-7.65 (m, IH), 7.63-7.58 (m, 1H), 7.41-7.34 (m, 1H), 7.31-7.23 (m, 1H), 6.68-6.61 (m, 1H), 4.56 (s, 2H), 4.05-3.96 (m, 2H), 3.75-3.61 (m, 14l H), 3.59-3.52 (m, 1H), 3.35-3.21 (m, 1H), 2.92-2.85 (m, 1H), 2.78-2.69 (m 1H), 2.48-2.39 (m, 1H), 1.82-1.71 (m 1H), 1.69-1.57 (m, 1H), 1.52-1.46 (m, 2H); LC/MS (ESI, m/z): [(M+H)]+=608.3.

Step 2-2-(5-(N-(2-(2-(2-(2-aminoethoxy) ethoxy) ethoxy)ethyl)-1-(isoquinolin-4-y1) piperidine-3-carboxamido)-2-oxopyridin-1 (2H)-yl) acetic acid. To a stirred mixture of 2-(5-(N-(2-(2-(2-(2-azidoethoxy) ethoxy) ethoxy)ethyl)-1-(isoquinolin-4-yl) piperidine-3-carboxamido)-2-oxopyridin-1 (2H)-yl) acetic acid (400 mg, 0.66 mmol) in THF (8 mL) and water (1 mL) was added triphenylphosphine (345.31 mg, 1.32 mmol) at rt under nitrogen atmosphere. The resulting mixture was stirred for 16 h at 50° C. under nitrogen atmosphere. On completion, the mixture was concentrated under reduced pressure. The residue was purified by reverse flash chromatography (column: WelFlash™ C18-I,20-40 um,120g; Eluent A: Water (plus 10 mmol/L TFA);Eluent B: ACN, 10% to 30% gradient in 25 min; Flow rate: 60 mL/min; detector: UV 220/254 nm, desired fractions were collected at 20% B and concentrated under reduced pressure) to afford the title compound (210 mg, 55% yield) as an yellow solid. LC/MS (ESI, m/z): [(M-H)]+=580.1.

### 2-(11-Bromoundecyl) isoindole-1,3-dione (Intermediate S)

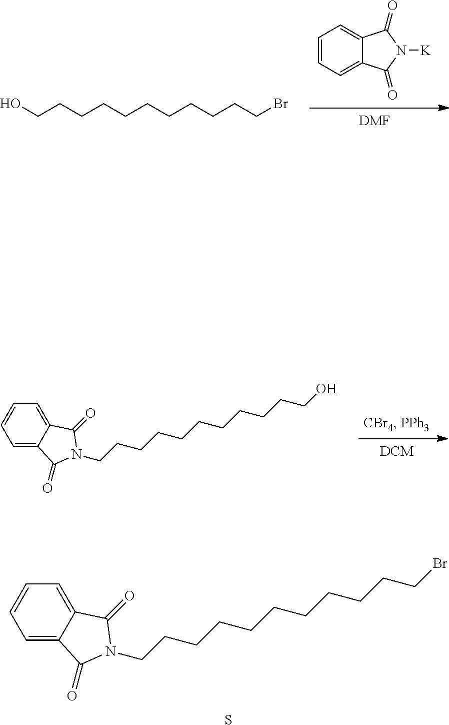

Step 1-2-(11-hydroxyundecyl) isoindole-1,3-dione. To a stirred mixture of 11-bromoundecan-1-ol (12 g, 48 mmol, CAS #1611-56-9) in DMF (100 mL) was added 2-potassioisoindole-1,3-dione (8.85 g, 47.8 mmol) at rt. The resulting mixture was stirred for 2 h at 70° C. On completion, the reaction mixture was diluted with water (1 L) and extracted with EtOAc (3×500 mL). The combined organic layer was dried over anhydrous Na₂SO₄. After filtration, the filtrate was concentrated under reduced pressure to afford the title compound (18.9 g, 88% yield) as a yellow solid. LC/MS (ESI, m/z): [(M+H)] +=318.2.

Step 2-2-(11-bromoundecyl) isoindole-1,3-dione. To a stirred mixture of 2-(11-hydroxyundecyl) isoindole-1,3-dione (18.8 g, 59.2 mmol) and PPh3 (23.30 g, 88.84 mmol) in DCM (800 mL) was added CBr4 (29.46 g, 88.84 mmol) at rt. The resulting mixture was stirred for 2 h at rt under nitrogen atmosphere. On completion, the mixture was concentrated under reduced pressure. The residue was purified by silica gel column chromatography, eluted with PE/EA (10:1), and concentrated under reduced pressure to afford the title compound (16.4 g, 56% yield) as a white solid. 1H NMR (300 MHz, Chloroform-d) δ 7.86 (t, J=5.6. 3.2 Hz, 2H), 7.72 (t, J=5.6, 3.2 Hz, 2H), 3.75-3.64 (m, 2H), 3.42 (t, J=6.8 Hz, 2H), 1.86 (p, J=7.2 Hz, 2H), 1.69 (p, J=7.2 Hz, 2H), 1.49-1.39 (m, 2H), 1.37-1.26 (m, 12l H); LC/MS (ESI, m/z): [(M+H)]+=380.0, 382.0.

### 2-(5-(N-(11-(1,3-dioxoisoindolin-2-yl) undecyl) piperidine-3-carboxamido)-2-oxopyridin-1 (2H)-yl) acetic acid (Intermediate T)

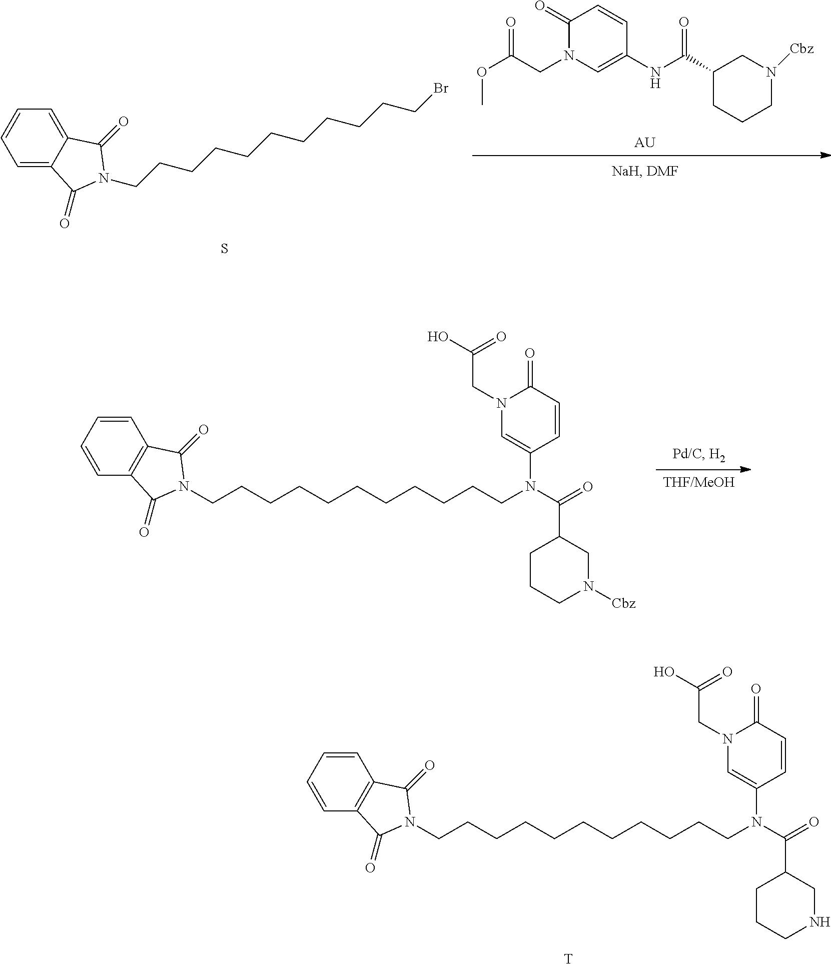

Step 1-2-(5-(1-((benzyloxy) carbonyl)-N-(11-(1,3-dioxoisoindolin-2-yl) undecyl) piperidine-3-carboxamido)-2-oxopyridin-1 (2H)-yl) acetic acid. To a stirred mixture of benzyl(S)-3-((1-(2-methoxy-2-oxoethyl)-6-oxo-1,6-dihydropyridin-3-yl) carbamoyl) piperidine-1-carboxylate (5.2 g, 12 mmol, Intermediate AU) in DMF (80 mL) was added NaHI (1.46 g, 36.5 mmol, 60% dispersion in mineral oil) in portions at 0° C. The resulting mixture was stirred for 30 min at rt under nitrogen atmosphere. To the above mixture was added a solution of 2-(11-bromoundecyl) isoindole-1,3-dione (9.25 g, 24.33 mmol, Intermediate S) in DMF (50 mL) at rt. The resulting mixture was stirred for an additional 16 h at rt. On completion, the mixture was concentrated under reduced pressure. The residue was purified by reverse flash chromatography (column: WelFlash™ C18-I,20-40 um,330g; Eluent A: Water (plus 10 mmol/L NH₄CO₃);Eluent B: ACN, 20% to 40% gradient in 25 min; Flow rate: 80 mL/min; detector: UV 220/254 nm, desired fractions were collected at 38% B) and concentrated under reduced pressure to afford the title compound (2.04 g, 21% yield) as a yellow oil. 1H NMR (300 MHz, Chloroform-d) δ 7.84 (t,.J=5.2, 3.2 Hz, 2H), 7.79-7.65 (m, 2H), 7.38-7.29 (m, 2H), 7.12 (m, 5H), 6.52-6.43 (m, 1H), 5.04 (s, 2H), 4.42 (s, 2H), 4.18-3.90 (m, 2H), 3.75-3.62 (m, 2H), 3.55-3.40 (m, 2H), 3.05-2.90 (m, 2H), 2.78-2.69 (m, 2H), 2.55-2.46 (m, 2H), 1.81-1.51 (m, 6H), 1.48-1.38 (m, 2H), 1.37-1.20 (m, 18l H); LC/MS (ESI, m/z): [(M+H)]+=713.3.

2-2-(5-(N-(11-(1,3-dioxoisoindolin-2-yl) undecyl) piperidine-3-carboxamido)-2-Step oxopyridin-1 (2H)-yl) acetic acid. To a stirred mixture of 2-(5-(1-((benzyloxy) carbonyl)-N-(11-(1,3-dioxoisoindolin-2-yl) undecyl) piperidine-3-carboxamido)-2-oxopyridin-1 (2H)-yl) acetic acid 1 g, 1 mmol) in THF (20 mL) and MeOH (20 mL) was added Pd/C (200 mg) at rt under nitrogen atmosphere. The resulting mixture was then stirred for 6 h at rt under hydrogen atmosphere (1 atm). The resulting mixture was filtered, and the filter cake was washed with THF (2×30 mL). The filtrate was concentrated under reduced pressure to the title compound (900 mg, 94% yield) as a yellow solid. 1H NMR (400 MHZ, DMSO-d₆) δ 7.89-7.79 (m, 3H), 7.66-7.54 (m, 2H), 7.35-7.28 (m, 1H), 6.43-6.02 (m, 1H), 4.42 (s, 2H), 4.18-3.90 (m, 2H), 3.75-3.62 (m, 2H), 3.55-3.40 (m, 2H), 3.05-2.90 (m, 2H), 2.78-2.69 (m, 2H), 2.55-2.46 (m, 2H), 1.81-1.51 (m, 4H), 1.48-1.38 (m, 2H), 1.37-1.20 (m, 14l H); LC/MS (ESI, m/z): [(M+H)]+=579.3.

### 2-(5-(N-(11-aminoundecyl)-1-(isoquinolin-4-yl) piperidine-3-carboxamido)-2-oxopyridin-1 (2H)-yl) acetic acid (Intermediate U)

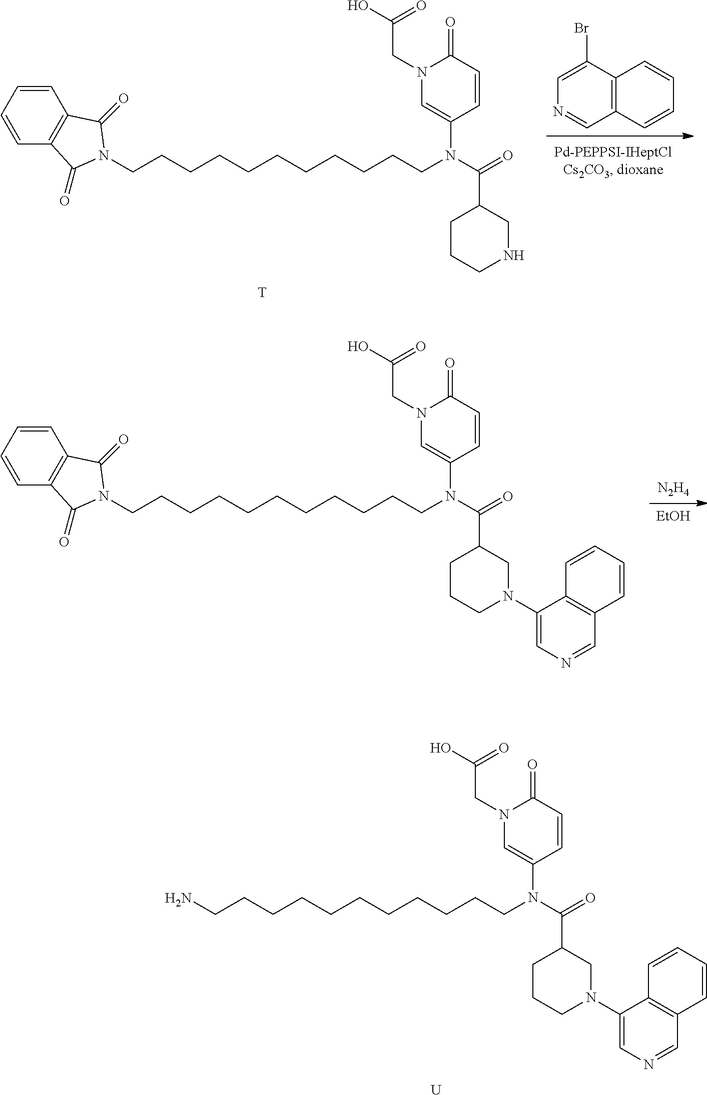

Step 1-2-(5-(N-(11-(1,3-dioxoisoindolin-2-yl) undecyl)-1-(isoquinolin-4-yl) piperidine-3-carboxamido)-2-oxopyridin-1 (2H)-yl) acetic acid. To a stirred mixture of 2-(5-(N-(11-(1,3-dioxoisoindolin-2-yl) undecyl) piperidine-3-carboxamido)-2-oxopyridin-1 (2H)-yl) acetic acid (1.0 g, 1.7 mmol, Intermediate T) in dioxane (25 mL) was added Cs₂CO₃ (1.13 g, 3.46 mmol) at rt under nitrogen atmosphere. Next, 4-bromoisoquinoline (0.36 g, 1.73 mmol) and Pd-PEPPSI-IHeptCl 3-chloropyridine (0.01 g, 0.09 mmol) was added to the mixture at rt. The resulting mixture was stirred for an additional 16 h at 80° C. On completion, the mixture was cooled to rt and concentrated under reduced pressure. The residue was purified by reverse flash chromatography (column: WelFlash™ C18-I,20-40 um,330g; Eluent A: Water (plus 10 mmol/L NH₄CO₃); Eluent B: ACN, 10% to 30% gradient in 25 min; Flow rate: 80 mL/min; detector: UV 220/254 nm, desired fractions were collected at 23% B) and concentrated under reduced pressure to afford the title compound (330 mg, 27% yield) as a yellow oil. LC/MS (ESI, m/z): [(M+H₂O)]+=724.5.

Step 2-2-(5-(N-(11-aminoundecyl)-1-(isoquinolin-4-yl) piperidine-3-carboxamido)-2-oxopyridin-1 (2H)-yl) acetic acid. To a stirred solution of (2-(5-(N-(11-(1,3-dioxoisoindolin-2-yl) undecyl)-1-(isoquinolin-4-yl) piperidine-3-carboxamido)-2-oxopyridin-1 (2H)-yl) acetic acid (300 mg, 0.43 mmol) in EtOH (15 mL) was added hydrazine hydrate (264 mg, 8.50 mmol) at 0°

C. under nitrogen atmosphere. The resulting mixture was stirred for 16 h at 80° C. under nitrogen atmosphere. On completion, the mixture cooled to rt and concentrated under reduced pressure. The residue was purified by reverse flash chromatography (column: WelFlash™ C18-I,20-40 um,120g; Eluent A: Water (plus 10 mmol/L TFA); Eluent B: ACN, 10% to 30% gradient in 25 min; Flow rate: 60 mL/min; detector: UV 220/254 nm, desired fractions were collected at 25% B) and concentrated under reduced pressure to afford the title compound (210 mg, 86% yield) as an yellow oil. 1H NMR (300 MHz, DMSO-d₆) δ 9.23 (s, 1H), 8.27 (d, J=8.0 Hz, 1H), 8.21-8.15 (m, 1H), 7.91 (d, J=3.2 Hz, 2H), 7.87-7.79 (m, 1H), 7.66-7.58 (m, 1H), 7.53 (d, J=9.6 Hz, 1H), 6.53 (d, J=9.6 Hz, 1H), 4.17 (s, 2H), 3.42-3.32 (m, 3H), 2.92-2.80 (m, 4H), 2.81-2.67 (m, 4H), 1.98-1.85 (m, 2H), 1.82-1.73 (m, 2H), 1.56-1.46 (m, 2H), 1.45-1.38 (m, 4H), 1.32-1.15 (m, 14l H); LC/MS (ESI, m/z): [(M+H)]+=576.4.

### 2-(14-Bromotetradecyl) isoindole-1,3-dione (Intermediate V)

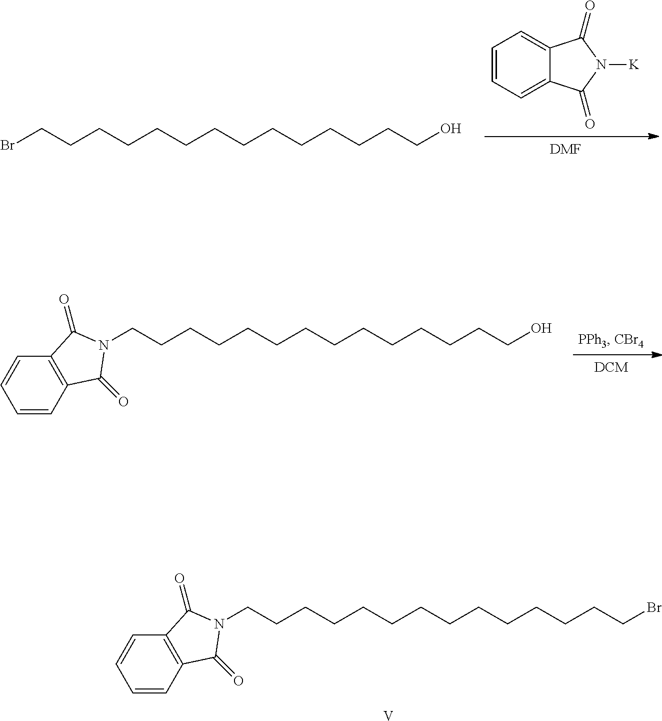

Step 1-2-(14-Hydroxytetradecyl) isoindole-1,3-dione. To a stirred solution of 14-bromotetradecan-1-ol (5 g, 17.0 mmol, CAS #72995-94-9) in DMF (100 mL) was added phthalimide (3.76 g, 25.5 mmol) at rt. The resulting mixture was stirred for 16 h at 70° C. under nitrogen atmosphere. On completion, the reaction mixture was cooled to rt and diluted with water (300 mL), then extracted with EtOAc (3×150 mL). The combined organic layers were washed with water (3×100 mL), and dried over anhydrous Na₂SO₄. After filtration, the filtrate was concentrated under reduced pressure to afford the title compound (6.4 g) as a white solid. LC/MS (ESI, m/z): [(M+1)]+=360.2; 1H NMR (400 MHZ, Chloroform-d) δ 7.86 (dd, J=5.4, 3.1 Hz, 2H), 7.73 (dd, J=5.5, 3.0 Hz, 2H), 3.73-3.64 (m, 4H), 1.72-1.51 (m, 4H), 1.40-1.21 (m, 20l H).

Step 2-2-(14-Bromotetradecyl) isoindole-1,3-dione. To a stirred solution of 2-(14-hydroxytetradecyl) isoindole-1,3-dione (6.4 g) and PPh3 (7.00 g, 26.7 mmol) in DCM (120 mL) was added CBr4 (8.86 g, 26.7 mmol) at rt under nitrogen atmosphere. The resulting mixture was stirred for 1 h at rt under nitrogen atmosphere. On completion, the mixture was concentrated under reduced pressure. The residue was purified by silica gel column chromatography, eluted with PE/EA (9:1), to afford the title compound (6 g, 80% yield) as a white solid. LC/MS (ESI, m/z): [(M+1)]+=422.2; 1H NMR (400 MHZ, Chloroform-d)δ 7.86 (dd, J=5.4, 3.1 Hz, 2H), 7.72 (dd, J=5.4, 3.1 Hz, 2H), 3.69 (t, J=7.0 Hz, 2H), 3.42 (t, J=6.9 Hz, 2H), 1.91-1.82 (m, 2H), 1.73-1.62 (m, 2H), 1.47-1.21 (m, 20l H).

### (5-{N-[14-(1,3-dioxoisoindol-2-yl)tetradecyl] piperidine-3-amido}-2-oxopyridin-1-yl) acetic acid (Intermediate W)

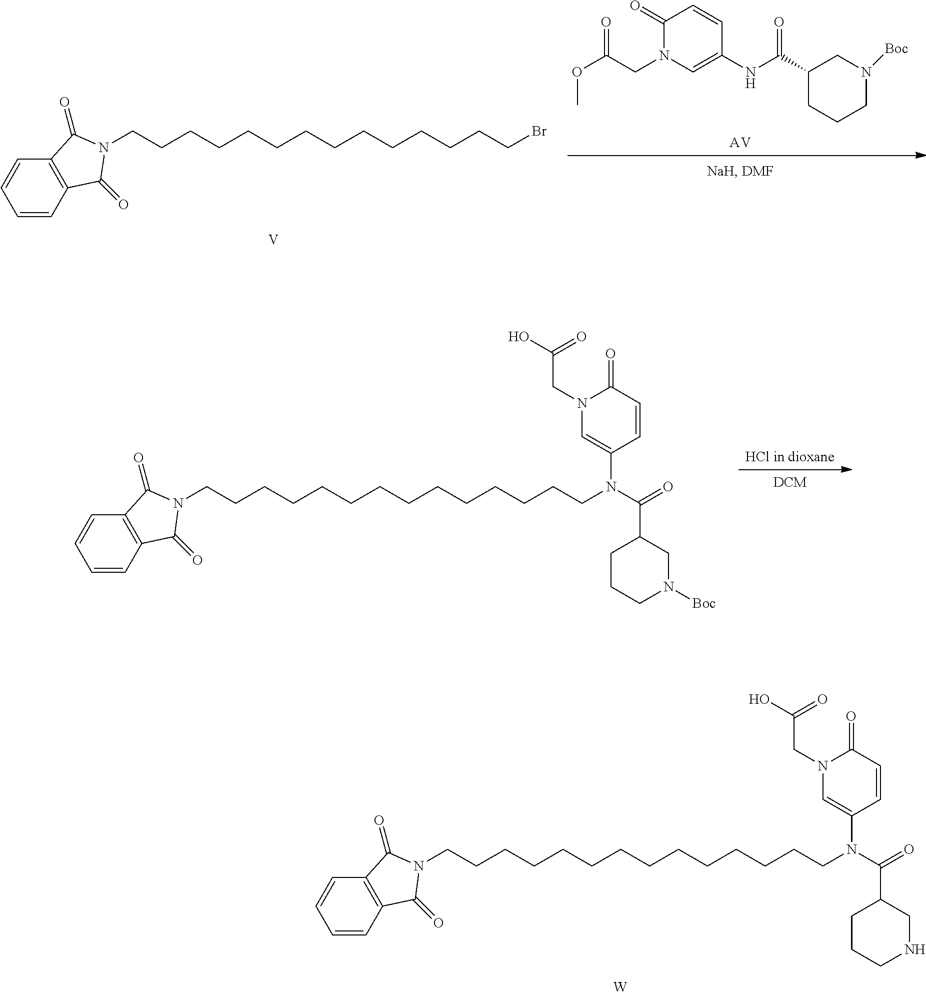

Step 1-(5-{N-[14-(1,3-dioxoisoindol-2-yl)tetradecyl] 1-(tert-butoxycarbonyl) piperidine-3-amido}-2-oxopyridin-1-yl) acetic acid. To a stirred solution of tert-butyl(S)-3-((1-(2-methoxy-2-oxoethyl)-6-oxo-1,6-dihydropyridin-3-yl) carbamoyl) piperidine-1-carboxylate (3.59 g, 9.4 mmol, Intermediate AV) in DMF (50 mL) was added NaH (1.14 g, 28.4 mmol, 60% dispersion in mineral oil) at 0° C. The resulting mixture was stirred for 30 min at rt. Next, 2-(14-bromotetradecyl) isoindole-1,3-dione (6 g, 14.2 mmol, Intermediate V) was added to the mixture at 0° C. The resulting mixture was stirred for an additional 16 h at 50° C. On completion, the mixture was cooled to rt and quenched with sat. NH₄Cl (aq.)(300 mL) at 0° C. The resulting mixture was extracted with EtOAc (3×150 mL). The combined organic layers were washed with water (3×200 mL), and dried over anhydrous Na₂SO₄. After filtration, the filtrate was concentrated under reduced pressure. The residue was purified by reverse phase flash chromatography (Column: WelFlash™ C18-I, 20-40 um, 330 g; Eluent A: Water (plus 10 mmol/L NH₄HCO3); Eluent B: ACN; Gradient: 20%-60% B in 40 min; Flow rate: 80 mL/min; Detector: 220/254 nm; desired fractions were collected at 33% B) and concentrated under reduced pressure to afford the title compound (which racemized during this step)(1.2 g, 18% yield) as a red oil. LC/MS (ESI, m/z): [(M+1)] =721.4; 1H NMR (400 MHZ, Chloroform-d)δ 7.85 (dd, J=5.4, 3.0 Hz, 2H), 7.72 (dd, J=5.4, 3.1 Hz, 2H), 7.44-7.32 (m, 1H), 7.26-7.26 (m, 1H), 6.64-6.55 (m, 1H), 4.66-4.38 (m, 2H), 4.08-3.86 (m, 2H), 3.74-3.63 (m, 2H), 3.62-3.44 (m, 2H), 2.95-2.81 (m, 1H), 2.76-2.60 (m, 1H), 2.50-2.36 (m, 1H), 1.81-1.59 (m, 4H), 1.54-1.17 (m, 33l H).

Step 2-(5-{N-[14-(1,3-dioxoisoindol-2-yl)tetradecyl] piperidine-3-amido}-2-oxopyridin-1-yl) acetic acid. To a stirred solution of (5-{N-[14-(1,3-dioxoisoindol-2-yl)tetradecyl] 1-(tert-butoxycarbonyl) piperidine-3-amido}-2-oxopyridin-1-yl) acetic acid (1.2 g, 1.6 mmol) in DCM (10 mL) was added HCl (gas) in 1,4-dioxane (10 mL, 4M) at rt. The resulting mixture was stirred for 1 h at rt. On completion, the reaction mixture was concentrated under reduced pressure. The residue was purified by reverse phase flash chromatography (Column: WelFlash™ C18-I, 20-40 um, 330 g; Eluent A: Water (plus 10 mmol/L NH₄HCO3); Eluent B: ACN; Gradient: 35%-80% B in 50 min; Flow rate: 80 mL/min; Detector: 220/254 nm; desired fractions were collected at 76% B) and concentrated under reduced pressure to afford the title compound (800 mg, 77% yield) as a yellow oil. LC/MS (ESI, m/z): [(M+1)]+=621.4; 1H NMR (400 MHZ, Chloroform-d)δ 7.86 (dd,.J=5.4, 3.0 Hz, 2H), 7.73 (dd, J=5.5, 3.0 Hz, 2H), 7.59-7.47 (m, 1H), 7.26-7.19 (m, 1H), 6.66-6.60 (m, 1H), 5.33-5.19 (m, 1H), 4.25-4.04 (m, 1H), 3.73-3.65 (m, 2H), 3.64-3.58 (m, 1H), 3.53-3.34 (m, 3H), 3.05-2.77 (m, 3H), 1.86-1.61 (m, 4H), 1.60-1.37 (m, 4H), 1.36-1.15 (m, 20l H).

### {5-[N-(14-Aminotetradecyl)₁-(isoquinolin-4-yl) piperidine-3-amido]-2-oxopyridin-1-yl} acetic acid (Intermediate X)

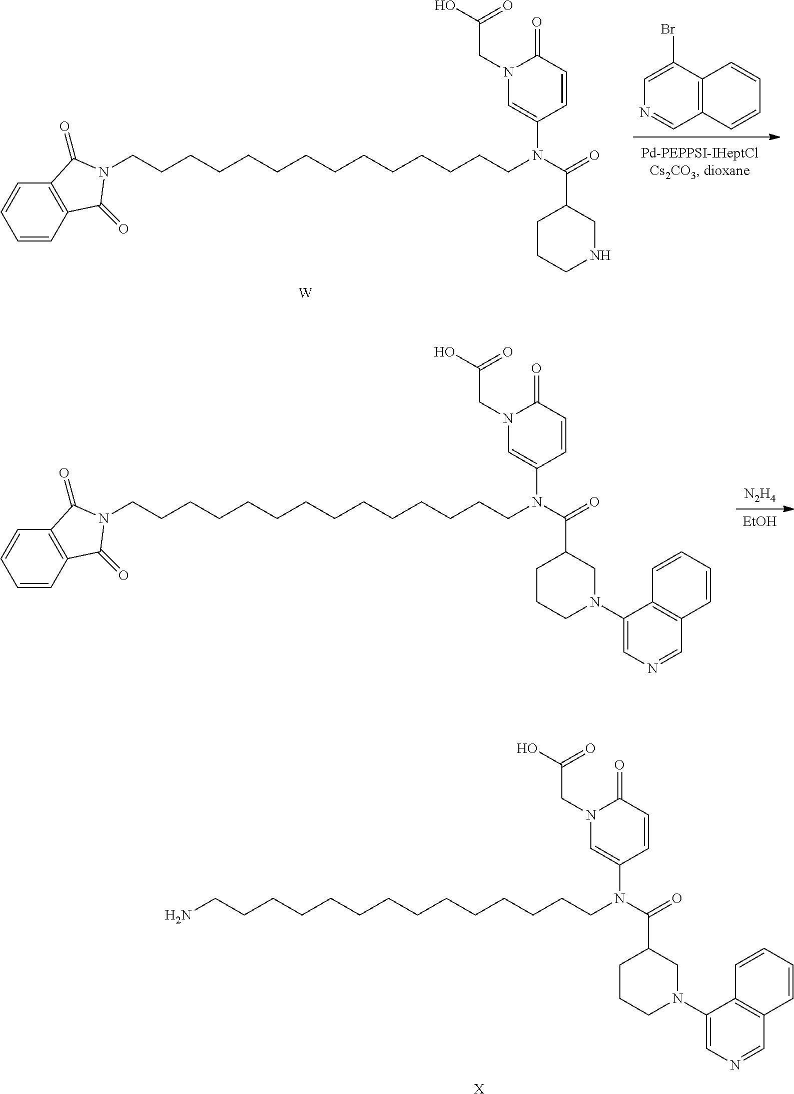

Step 1-(5-{N-[14-(1,3-dioxoisoindol-2-yl)tetradecyl] 1-(isoquinolin-4-yl) piperidine-3-amido}-2-oxopyridin-1-yl) acetic acid. To a stirred solution of (5-{N-[14-(1,3-dioxoisoindol-2-yl)tetradecyl] piperidine-3-amido}-2-oxopyridin-1-yl) acetic acid (770 mg, 1.2 mmol, Intermediate W) and Cs₂CO₃ (1200 mg, 3.7 mmol) in dioxane (16 mL) were added Pd-PEPPSI-IHeptCl 3-chloropyridine (121 mg, 0.10 mmol) and 4-bromoisoquinoline (310 mg, 1.4 mmol) at rt. The resulting mixture was stirred for 16 h at 80° C. under nitrogen atmosphere. On completion, the reaction mixture was cooled to rt and concentrated under reduced pressure. The residue was purified by reverse phase flash chromatography (Column: WelFlash™ C18-I, 20-40 um, 330 g; Eluent A: Water (plus 10 mmol/L FA); Eluent B: ACN; Gradient: 25%-65% B in 40 min; Flow rate: 80 mL/min; Detector: 220/254 nm; desired fractions were collected at 53% B) and concentrated under reduced pressure to afford the title compound (600 mg, 65% yield) as a brown-yellow oil. LC/MS (ESI, m/z): [(M+1)]+=748.4; 1H NMR (400 MHZ, Chloroform-d)_{8 9.44-9.35} (m, 1H), 8.36-8.26 (m, 1H), 8.22-8.11 (s, 1H), 8.08-8.00 (m, 1H), 7.94-7.88 (m, 1H), 7.86 (dd, J=5.5, 3.0 Hz, 2H), 7.73 (dd, J=5.4, 3.1 Hz, 2H), 7.52-7.43 (m, 1H), 7.41-7.32 (m, 1H), 7.28-7.26 (m, 1H), 6.97-6.79 (m, 1H), 5.82-5.68 (m, 1H), 4.27-4.14 (m, 1H), 3.91-3.79 (m, 1H), 3.74-3.59 (m, 4H), 3.57-3.33 (m, 3H), 3.10-2.91 (m, 1H), 1.97-1.85 (m, 2H), 1.77-1.59 (m, 3H), 1.58-1.40 (m, 3H), 1.29 (d, J=34.2 Hz, 20l H).

Step 2-{5-[N-(14-aminotetradecyl)₁-(isoquinolin-4-yl) piperidine-3-amido]-2-oxopyridin-1-yl} acetic acid. To a stirred solution of (5-{N-[14-(1,3-dioxoisoindol-2-yl)tetradecyl] 1-(isoquinolin-4-yl) piperidine-3-amido}-2-oxopyridin-1-yl) acetic acid (600 mg, 0.8 mmol) in EtOH (12 mL) was added hydrazine hydrate (2 mL, 98% solution) at rt. The resulting mixture was stirred for 1 h at 70° C. under nitrogen atmosphere. On completion, the reaction mixture was cooled to rt and concentrated under reduced pressure. The residue was purified by reverse phase flash chromatography (Column: WelFlash™ C18-I, 20-40 um, 330 g; Eluent A: Water (plus 10 mmol/L TFA); Eluent B: ACN; Gradient: 15%-55% B in 40 min; Flow rate: 80 mL/min; Detector: 220/254 nm; desired fractions were collected at 35% B) and concentrated under reduced pressure to afford the title compound (340 mg, 69% yield) as a yellow oil. LC/MS (ESI, m/z): [(M+1)]+=618.4; 1H NMR (400 MHZ, DMSO-d₆) δ 9.42-9.25 (m, 1H), 8.43-8.30 (m, 1H), 8.24-8.16 (m, 1H), 8.02-7.85 (m, 3H), 7.78-7.60 (m, 3H), 7.57-7.47 (m, 1H), 6.53 (d, J=9.6 Hz, 1H), 4.66-4.48 (m, 2H), 3.74-3.52 (m, 2H), 3.42-3.29 (m, 2H), 2.98-2.83 (m, 3H), 2.80-2.71 (m, 2H), 1.96-1.87 (m, 1H), 1.84-1.75 (m, 1H), 1.74-1.56 (m, 2H), 1.55-1.47 (m, 2H), 1.45-1.34 (m, 2H), 1.35-1.08 (m, 20l H).

### (S)-2-(5-(N-(2-(2-(2-(2-Aminoethoxy) ethoxy) ethoxy)ethyl)-1-(isoquinolin-4-yl) piperidine-3-carboxamido)-2-oxopyridin-1 (2H)-yl) acetic acid (Intermediate Y)

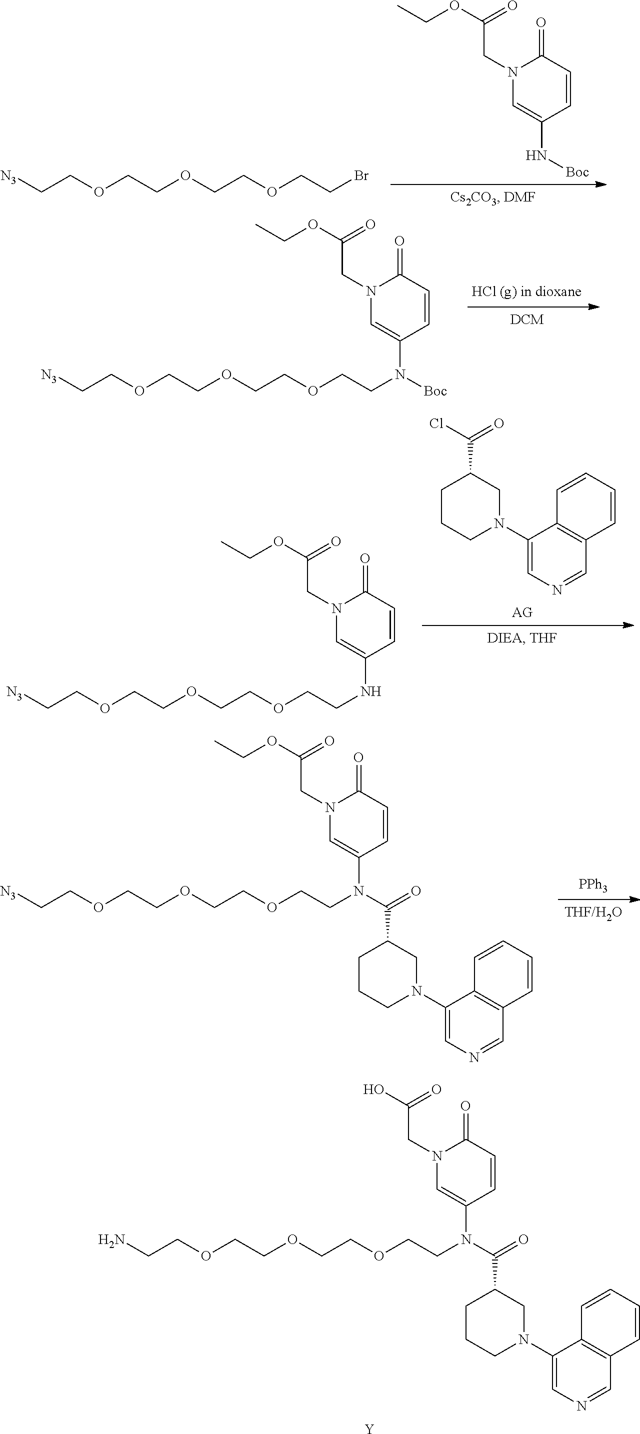

Step 1-Ethyl 2-(5-((2-(2-(2-(2-azidoethoxy) ethoxy) ethoxy)ethyl)(tert-butoxycarbonl)amino)-2-oxopyridin-1 (2H)-yl)acetate. A mixture of 1-azido-2-(2-(2-(2-bromoethoxy) ethoxy) ethoxy) ethane (4.0 0 g, 14.2 mmol, CAS #1446282-43-4), ethyl 2-(5-((tert-butoxycarbonyl)amino)-2-oxopyridin-1 (2H)-yl) ac etate (3.50 g, 11.8 mmol, synthesized via Steps 1-2 of Intermediate A) and Cs₂CO₃ (11.55 g, 35.44 mmol) in DMF (60 mL) was stirred for 16 hr at 50° C. On completion, the mixture was cooled to rt and purified d irectly by reversed-phase flash chromatography (Column: Spherical C18, 20˜40 μm, 330 g; Mobile Phase A: Water (plus 0.1% HCOOH), Mobile Phase B: acetonitrile; Flow rate: 80 mL/min; Gradient (B %): 5˜ 30%, 4 min; 30% ˜50%, 20 min; 50% ˜95%; 2 min; 95%, 5 min; Detector: 254 nm; the fractions containin g desired product were collected at 40% B) and concentrated under reduced pressure to afford the title co mpound (4 g, 68% yield) as a green oil. 1H NMR (400 MHZ, CDC13)δ 7.49-7.33 (m, 2H), 6.54 (d, J=9.6 Hz, 1H), 4.64 (s, 2H), 4.24 (q, J=7.2 Hz, 2H), 3.76-3.54 (m, 14l H), 3.41-3.37 (m, 2H), 1.44 (s, 9H), 1.3 0 (t, J=7.2 Hz, 3H); LC/MS (ESI, m/z): [(M+1)]+=498.2.

Step 2-Ethyl 2-(5-((2-(2-(2-(2-azidoethoxy) ethoxy) ethoxy)ethyl) amino)-2-oxopyridin-1 (2H)-yl)acetate. To a stirred mixture of ethyl 2-(5-((2-(2-(2-(2-azidoethoxy) ethoxy) ethoxy)ethyl)(tert-butoxycarbonyl)amino)-2-oxopyridin-1 (2H)-yl)acetate (4 g, 8 mmol) in DCM (20 mL) was added HCl (gas) in 1,4-dioxane (20 mL) dropwise at rt. The resulting mixture was stirred for 1 h at rt. On completion, the mixture was concentrated under reduced pressure to afford the title compound (2.8 g, 88% yield) as a green oil. 1H NMR (400 MHZ, DMSO-d₆) δ 7.72 (s, 1H), 7.62-7.57 (m, 1H), 6.53 (dd, J=9.6, 1.6 Hz, 1H), 4.71 (s, 2H), 4.16 (q, J=7.2 Hz, 2H), 3.66 (t, J=5.2 Hz, 2H), 3.64-3.51 (m, 12l H), 3.44-3.37 (m, 2H), 1.22 (t, J=7.2 Hz, 3H). LC/MS (ESI, m/z): [(M+1)]+=398.2.

Step 3-Ethyl(S)-2-E (5-(N-(2-(2-(2-(2-azidoethoxy) ethoxy) ethoxy)ethyl)-1-(isoquinolin-4-y1) piperidine-3-carboxamido)-2-oxopyridin-1 (2/)-yl)acetate. To a stirred mixture of ethyl 2-(5-((2-(2-(2-(2-azidoethoxy) ethoxy) ethoxy)ethyl)amino)-2-oxopyridin-1 (2H)-yl)acetate (2.00 g, 5.03 mmol) and(S)-1-(isoquinolin-4-yl) piperidine-3-carbonyl chloride (1.66 g, 6.04 mmol, Intermediate AG) in THF (30 mL) was added DIEA (1.95 g, 15.10 mmol) dropwise at 0° C. The resulting mixture was then stirred for 1 h at rt. On completion, the mixture was purified by reversed-phase flash chromatography (Column: Spherical C18, 20˜40 μm, 330 g; Mobile Phase A: Water (plus 0.1% HCOOH), Mobile Phase B: acetonitrile; Flow rate: 80 mL/min; Gradient (B %): 5%˜20%, 4 min; 20% ˜40%, 20 min; 40% ˜95%; 2 min; 95%, 5 min; Detector: 254 nm; the fractions containing desired product were collected at 27% B) and concentrated under reduced pressure to afford the title compound (1.20 g, 38% yield) as a green oil. LC/MS (ESI, m/z): [(M+1) |+=636.3.

Step 4-(S)-2-(5-(N-(2-(2-(2-(2-aminoethoxy) ethoxy) ethoxy)ethyl)-1-(isoquinolin-4-yl) piperi dine-3-carboxamido)-2-oxopyridin-1 (2H)-yl) acetic acid. A mixture of ethyl(S)-2-(5-(N-(2-(2-(2-(2-azido ethoxy) ethoxy) ethoxy)ethyl)-1-(isoquinolin-4-yl) piperidine-3-carboxamido)-2-oxopyridin-1 (2H)-yl) aceta the (1.10 g, 1.73 mmol) and PPh3 (908 mg, 3.46 mmol) in THF (16 mL) and H₂O (4 mL) was stirred for 16 hr at 50° C. The mixture was cooled to rt and purified by reversed-phase flash chromatography (Column: Spherical C18, 20˜40 μm, 330 g; Mobile Phase A: Water (plus 0.1% HCOOH), Mobile Phase B: acetonitr ile; Flow rate: 80 mL/min; Gradient (B %): 5%˜10%, 4 min; 10% ˜20%, 20 min; 20% ˜95%; 2 min; 95%, 5 min; Detector: 254 nm; the fractions containing desired product were collected at 14% B) and concentrate d under reduced pressure to afford the title compound (600 mg, 60% yield) as a yellow oil. LC/MS (ESI, m/z): [(M+1)]+=582.3.

### Tert-butyl ((5S,8S,10aR)-8-(((S)-5-amino-1-(benzhydrylamino)-1,5-dioxopentan-2-yl) carbamoyl)-6-oxodecahydropyrrolo[1,2-a][1,5] diazocin-5-yl) carbamate (Intermediate Z)

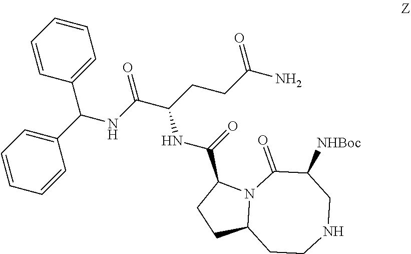 [SMILES: CC(C)(C)OC(=O)N[C@H]1CNCC[C@H]2CC[C@@H](C(=O)N[C@@H](CCC(N)=O)C(=O)NC(c3ccccc3)c3ccccc3)N2C1=O]

This compound was prepared as described in WO2021/188696 (Intermediate O).

### Perfluorophenyl 5-((diethoxyphosphoryl) carbonyl)-1H-indole-2-carboxylate (Intermediate A A)

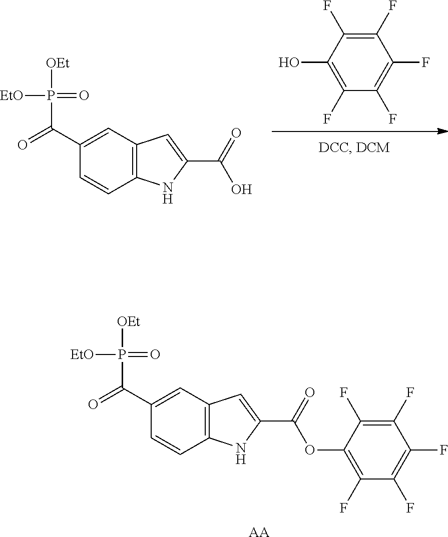 [SMILES: CCOP(=O)(OCC)C(=O)c1ccc2[nH]c(C(=O)O)cc2c1 ; Oc1c(F)c(F)c(F)c(F)c1F ; CCOP(=O)(OCC)C(=O)c1ccc2[nH]c(C(=O)Oc3c(F)c(F)c(F)c(F)c3F)cc2c1 ; "AA"]

To a stirred mixture of 5-((diethoxyphosphoryl) carbonyl)-1H-indole-2-carboxylic acid (3.00 g, 9.23 mmol, synthesis described in WO2021/188696) and 2,3,4,5,6-pentafluorophenol (1.87 g, 10.2 mmo 1) in DCM (50 mL) was added DCC (2.85 g, 13.9 mmol) at rt under nitrogen atmosphere. The resulting mi xture was then stirred for 16 hr at rt under nitrogen atmosphere. On completion, the reaction mixture was f iltered, and the filter cake was washed with DCM (3×20 mL). The filtrate was concentrated under reduce d pressure and the residue was diluted with hexane (50 mL). The precipitated solids were collected by filtr ation and washed with hexane (3×20 mL), then dried to afford the title compound (4 g, 88% yield) as a li ght yellow solid. 1H NMR (400 MHZ, DMSO-d₆) δ 13.02 (s, 1H), 8.97-8.84 (m, 1H), 8.06 (dd, J=8.8,1. 6 Hz, 1H), 7.94 (s, 1H), 7.75-7.60 (m, 1H), 4.26-4.18 (m, 4H), 1.30 (t, J=7.2 Hz, 6H); LC/MS (ESI, m iz): [(M+1)]+=492.1.

### 2-(5-((S)—N-(1-((5S,8S,10aR)-5-amino-8-(((S)-5-amino-1-(benzhydrylamino)-1,5-dioxopentan-2-yl) carbamoyl)-6-oxooctahydropyrrolo[1,2-a][1,5] diazocin-3 (4H)-yl)-1-oxo-5,8,11-trioxa-2-azatridecan-13-yl)-1-(isoquinolin-4-yl) piperidine-3-carboxamido)-2-oxopyridin-1 (2H)-yl) acetic acid (Intermediate A B)

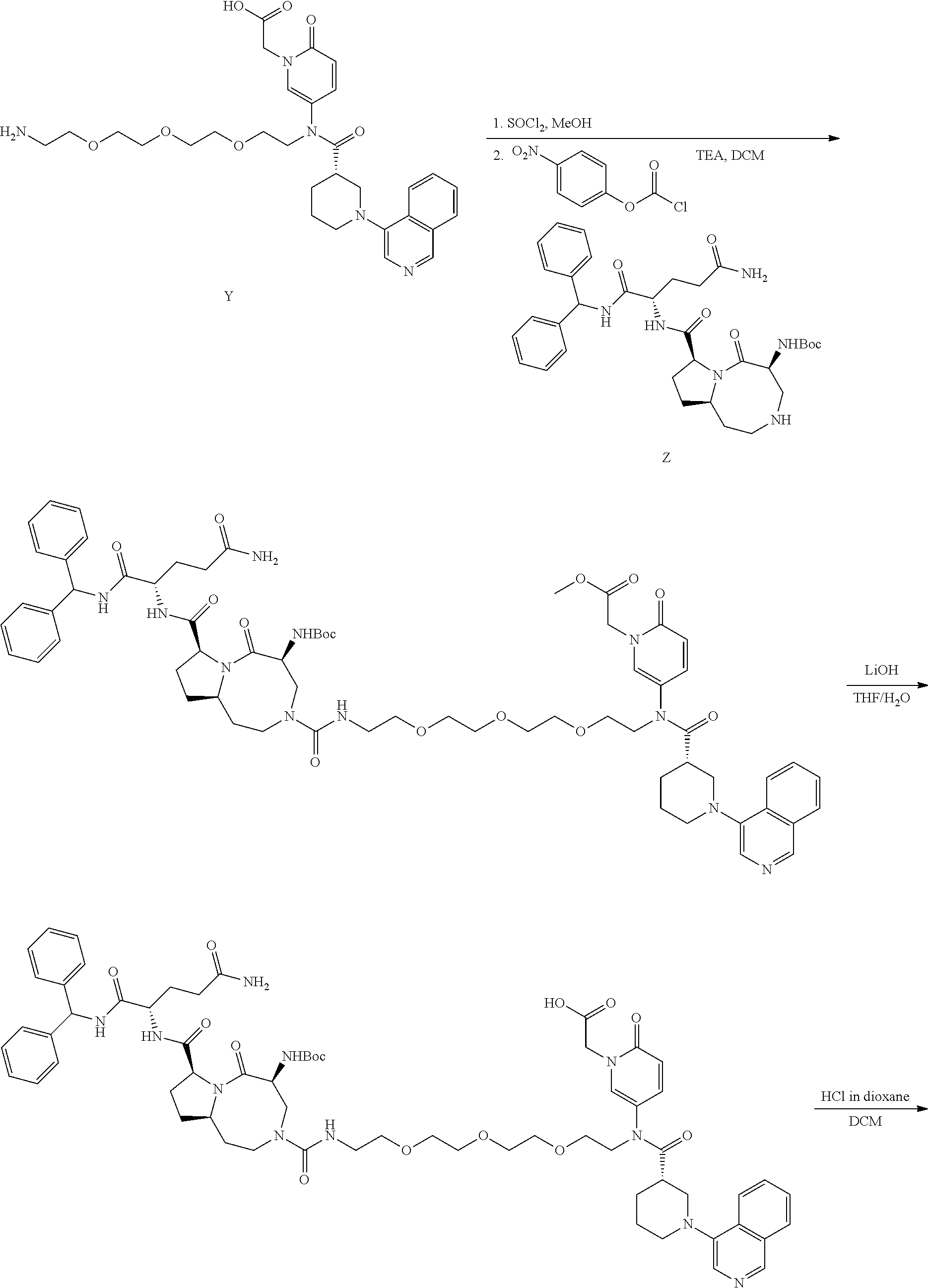

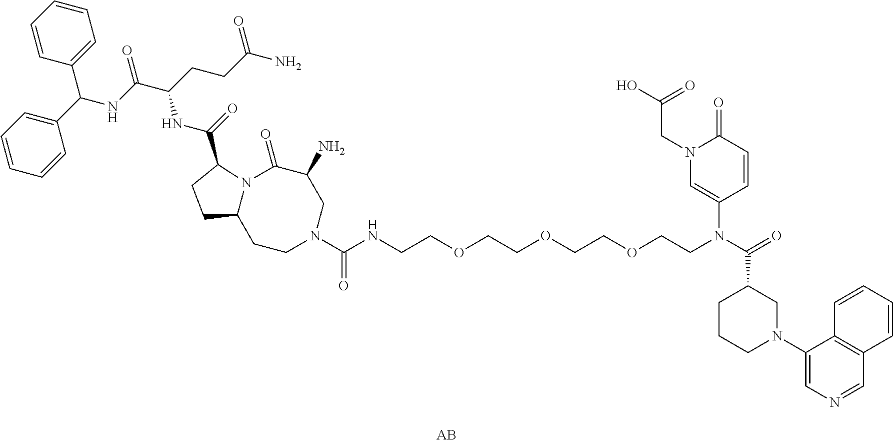 [SMILES: NC(=O)CC[C@H](NC(=O)[C@@H]1CC[C@@H]2CCN(C(=O)NCCOCCOCCOCCN(C(=O)[C@H]3CCCN(c4cncc5ccccc45)C3)c3ccc(=O)n(CC(=O)O)c3)C[C@H](N)C(=O)N21)C(=O)NC(c1ccccc1)c1ccccc1]

Step 1-Methyl 2-(5-((S)—N-(1-((5S,8S,10aR)-8-(((S)-5-amino-1-(benzhydrylamino)-1,5-dioxopentan-2-yl) carbamoyl)-5-((tert-butoxycarbonyl)amino)-6-oxooctahydropyrrolo[1,2-a] [1,5] diazocin-3 (4H)-yl)-1-oxo-5,8,11-trioxa-2-azatridecan-13-yl)-1-(isoquinolin-4-yl) piperidine-3-carboxamido)-2-oxopyridin-1 (2H)-yl)acetate. To a stirred solution of(S)-2-(5-(N-(2-(2-(2-(2-aminoethoxy) ethoxy) ethoxy)ethyl)-1-(isoquinolin-4-yl) piperidine-3-carboxamido)-2-oxopyridin-1 (2H)-yl) acetic acid (450 mg, 0.77 mmol, Intermediate Y) in MeOH (10 mL) was added SOCI2 (368 mg, 3.09 mmol) dropwise at rt under air atmosphere. The resulting mixture was stirred for 1 hr at 70° C. The mixture was allowed to cool to rt and the mixture was concentrated under reduced pressure. The residue was dissolved in DCM (10 mL), then 4-nitrophenyl carbonochloridate (172 mg, 0.85 mmol) was added dropwise at 0° C. The resulting mixture was stirred for 1 hr at rt. Next, tert-butyl ((55,8S,10aR)-8-(((S)-5-amino-1-(benzhydrylamino)-1,5-dioxopentan-2-yl) carbamoyl)-6-oxodecahydropyrrolo[1,2-a][1,5] diazocin-5-yl) carbamate (480 mg, 0.77 mmol, Intermediate Z) was added in portions at rt. The resulting mixture was stirred for additional 16 hr at rt. On completion, the mixture was concentrated under reduced pressure. The crude product was purified by reverse phase Flash chromatography (column: WelFlash™ C18-I, 20-40 um, 330 g; Eluent A: Water (plus 10 mmol/L FA); Eluent B: acetonitrile; Gradient: 25%-55% B in 25 min; Flow rate: 80 mL/min; Detector: 254 nm; desired fractions were collected at 40% B) and concentrated under reduced pressure to afford the title compound (400 mg, 42% yield) as a yellow oil. LC/MS (ESI, m/z): [(M+1)]+=1242.7.

Step 2-2-(5-((S)—N-(1-((5S,8S,10aR)-8-(((S)-5-amino-1-(benzhydrylamino)-1,5-dioxopentan-2-yl) carbamoyl)-5-((tert-butoxycarbonyl)amino)-6-oxooctahydropyrrolo[1,2-a][1,5] diazocin-3 (4H)-yl)-1-oxo-5,8,11-trioxa-2-azatridecan-13-yl)-1-(isoquinolin-4-yl) piperidine-3-carboxamido)-2-oxopyridin-1 (2H)-yl)acetic acid. To a stirred mixture of methyl 2-(5-((S)—N-(1-((5S,8S,10aR)-8-(((S)-5-amino-1-(benzhydr ylamino)-1,5-dioxopentan-2-yl) carbamoyl)-5-((tert-butoxycarbonyl)amino)-6-oxooctahydropyrrolo[1,2-a] [1,5] diazocin-3 (4/)-yl)-1-oxo-5,8,11-trioxa-2-azatridecan-13-yl)-1-(isoquinolin-4-yl) piperidine-3-carbo xamido)-2-oxopyridin-1 (2H)-yl)acetate (380 mg, 0.31 mmol) in THF (8 mL) and H₂O (8 mL) was added LiOH (30 mg, 1.22 mmol) in portions at rt. The resulting mixture was stirred for 1 hr at rt. On completion, the residue was purified by reversed-phase flash chromatography (Column: Spherical C18, 20˜40 μm, 330 g; Mobile Phase A: Water (plus 0.1% HCOOH), Mobile Phase B: acetonitrile; Flow rate: 80 mL/min; Gra dient (B %): 5%˜22%, 4 min; 22% ˜40%, 20 min; 40% ˜95%; 2 min; 95%, 5 min; Detector: 254 nm; the fr actions containing desired product were collected at 39% B) and concentrated under reduced pressure to a fford the title compound (350 mg, 94% yield) as a yellow solid. 1H NMR (400 MHZ, DMSO-d₆) δ 8.99 (s, 1H), 8.72 (d, J=8.4 Hz, 1H), 8.21 (d, J=8.0 Hz, 1H), 8.17 (s, 2H), 8.14 (s, 1H), 8.08 (d, J=8.0 Hz, 1H), 7.92 (s, 1H), 7.85-7.53 (m, 4H), 7.38-7.20 (m, 11l H), 6.75 (s, 1H), 6.57-6.50 (m, 2H), 6.48-6.44 (m, 1H), 6.10 (d, J=8.4 Hz, 1H), 4.80-4.65 (m, 2H), 4.40 (t, J=8.8 Hz, 1H), 4.35-4.31 (m, 1H), 4.27-4.24 (m, 1 H), 4.04-3.99 (m, 1H), 3.86-3.56 (m, 10l H), 3.52-3.35 (m, 10l H), 3.31-3.26 (m, 3H), 3.22-3.09 (m, 1H), 2.93-2.88 (m, 1H), 2.84-2.76 (m, 3H), 2.19-2.06 (m, 3H), 1.98-1.86 (m, 2H), 1.87-1.70 (m, 1H), 1.66-1. 60 (m, 2H), 1.55-1.47 (m, 1H), 1.38 (s, 9H). LC/MS (ESI, m/z): [(M+1)]+=1228.7.

Step 3-2-(5-((S)—N-(1-((5S,8S,10aR)-5-amino-8-(((S)-5-amino-1-(benzhydrylamino)-1,5-dio xopentan-2-yl) carbamoyl)-6-oxooctahydropyrrolo[1,2-a][1,5] diazocin-3 (4H)-yl)-1-oxo-5,8,11-trioxa-2-az atridecan-13-yl)-1-(isoquinolin-4-yl) piperidine-3-carboxamido)-2-oxopyridin-1 (2H)-yl) acetic acid. To a s tirred mixture of 2-(5-((S)—N-(1-((5S,8S,10aR)-8-(((S)-5-amino-1-(benzhydrylamino)-1,5-dioxopentan-2-y 1) carbamoyl)-5-((tert-butoxycarbonyl)amino)-6-oxooctahydropyrrolo[1,2-a][1,5] diazocin-3 (4H)-yl)-1-ox 0-5,8,11-trioxa-2-azatridecan-13-yl)-1-(isoquinolin-4-yl) piperidine-3-carboxamido)-2-oxopyridin-1 (2H)-yl) acetic acid (350 mg, 0.28 mmol) in DCM (7 mL) was added HCl (gas) in 1,4-dioxane (7 mL) dropwise at rt. The resulting mixture was stirred for 1 hr at rt. On completion, the mixture was concentrated under r educed pressure to afford the title compound (300 mg, 94% yield) as a yellow solid. LC/MS (ESI, m/z): [(M+1)]+=1128.6.

### 2-(11-Bromoundecyl) isoindoline-1,3-dione (Intermediate AC)

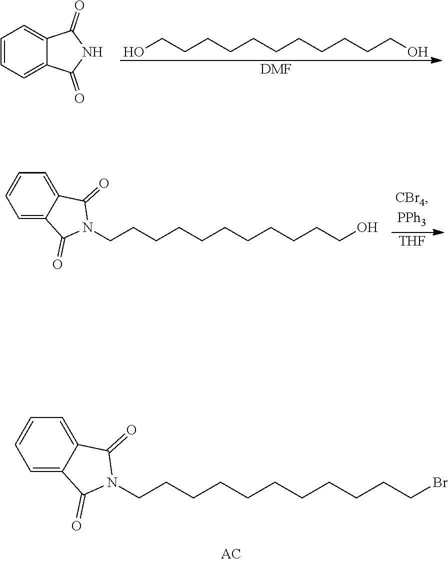

Step 1-2-(11-Hydroxyundecyl) isoindoline-1,3-dione. A mixture of isoindoline-1,3-dione (11 g, 60 mmol) and undecane-1,11-diol (14.92 g, 59.39 mmol) in DMF (60 mL) was stirred for 2 h at rt. On completion, the mixture was diluted with water (180 mL) and extracted with EtOAc (3×100 mL). The combined organic layers were washed with brine (3×200 mL), and dried over anhydrous Na₂SO₄. The resulting mixture was concentrated under reduced pressure. The residue was purified by silica gel column chromatography, eluted with CH₂Cl2/EA (10:1), to afford the title compound (10 g, 46%) as a white solid. 1H NMR (400 MHz, DMSO-d₆) δ 7.91-7.77 (m, 4H), 4.30 (t, J=5.2 Hz, 1H), 3.55 (t, J=7.2 Hz, 2H), 3.42-3.29 (m, 2H), 1.63-1.50 (m, 2H), 1.43-1.32 (m, 2H), 1.31-1.14 (m, 14l H); LC/MS (ESI, m/z): [(M+1)]+=318.4.

Step 2-2-(11-Bromoundecyl) isoindoline-1,3-dione. To a stirred solution of 2-(11-hydroxyundecyl) isoindoline-1,3-dione (10.0 g, 31.5 mmol) and PPh3 (12 g, 47.26 mmol) in THF (100 mL) was added CBr4 (16 g, 47 mmol) in portions at 0° C. under air atmosphere. The resulting mixture was then stirred for 2 hr at rt. On completion, the mixture was concentrated under reduced pressure. The residue was purified by silica gel column chromatography, eluted with CH₂Cl2/EA (15:1), to afford the title compound (10.5 g, 88% yield) as a white solid. 1H NMR (300 MHz, CDCl₃) δ 7.93-7.81 (m, 2H), 7.78-7.67 (m, 2H), 3.75-3.64 (m, 2H), 3.42 (t, J=6.8 Hz, 2H), 1.87 (p, J=6.8 Hz, 2H), 1.76-1.63 (m, 2H), 1.50-1.24 (m, 14l H); LC/MS (ESI, m/z): [(M+1)]+=380.1.

### Ethyl 2-(5-((11-(1,3-dioxoisoindolin-2-yl) undecyl)amino)-2-oxopyridin-1 (2H)-yl)acetate (Intermediate AD)

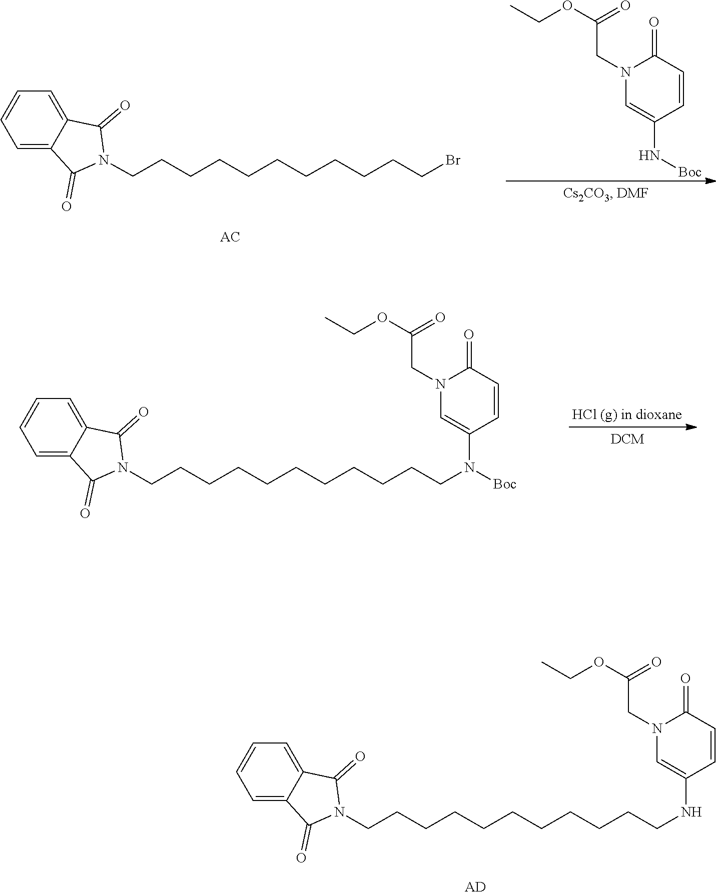 [SMILES: O=C1c2ccccc2C(=O)N1CCCCCCCCCCCBr ; CCOC(=O)Cn1cc(NC(=O)OC(C)(C)C)ccc1=O ; CCOC(=O)Cn1cc(N(CCCCCCCCCCCN2C(=O)c3ccccc3C2=O)C(=O)OC(C)(C)C)ccc1=O ; CCOC(=O)Cn1cc(NCCCCCCCCCCCN2C(=O)c3ccccc3C2=O)ccc1=O ; "AD"]

Step 1-Ethyl 2-(5-((tert-butoxycarbonyl)(11-(1,3-dioxoisoindolin-2-yl) undecyl)amino)-2-oxopyridin-1 (2H)-yl)acetate. To a stirred solution of 2-(11-bromoundecyl) isoindoline-1,3-dione (4.62 g, 12.15 mmol, Intermediate AC) and ethyl 2-(5-((tert-butoxycarbonyl)amino)-2-oxopyridin-1 (2H)-yl)acetate (3.00 g, 10.1 mmol, synthesized via Steps 1-2 of Intermediate A) in DMF (45 mL) was added Cs₂CO₃ (10.56 g, 32.40 mmol) in portions at rt under nitrogen atmosphere. The resulting mixture was then stirred for 16 hr at 50° C. On completion, the mixture was concentrated under reduced pressure. The crude product was purified by reverse phase flash chromatography (column: WelFlash™ C18-I, 20-40 um, 330 g; Eluent A: Water (plus 10 mmol/L FA); Eluent B: acetonitrile; Gradient: 50%-80% B in 25 min; Flow rate: 80 mL/min; Detector: 220/254 nm; desired fractions were collected at 72% B) and concentrated under reduced pressure to afford the title compound (4.8 g, 75%) as a brown solid. 1H NMR (400 MHZ, CDC13) 8 7.88-7.82 (m, 2H), 7.76-7.69 (m, 2H), 7.28-7.21 (m, 1H), 7.14 (s, 1H), 6.59 (d, J=9.6 Hz, 1H), 4.64 (s, 2H), 4.25 (q, J=7.2 Hz, 2H), 3.68 (t, J=7.2 Hz, 2H), 3.55-3.41 (m, 2H), 2.02 (s, 3H), 1.73-1.61 (m, 2H), 1.56-1.49 (m, 2H), 1.44 (s, 9H), 1.36-1.19 (m, 14l H); LC/MS (ESI, m/z): [(M+1)]+=596.3.

Step 2-Ethyl 2-(5-((11-(1,3-dioxoisoindolin-2-yl) undecyl)amino)-2-oxopyridin-1 (2H)-yl)acetate hydrochloride. To a stirred solution of ethyl 2-(5-((tert-butoxycarbonyl)(11-(1,3-dioxoisoindolin-2-yl) undecyl)amino)-2-oxopyridin-1 (2H)-yl)acetate (2.3 g, 3.9 mmol) in DCM (30 mL) was added HCl (gas) in 1,4-dioxane (30 mL) in portions at rt under air atmosphere. The resulting mixture was then stirred for 1 h at rt. On completion, the mixture was concentrated under reduced pressure to give the title compound (1.9 g, 98% yield) as a yellow solid. 1H NMR (400 MHZ, CDC13)δ 7.94-7.80 (m, 3H), 7.73-7.71 (m, 2H), 7.57 (dd, J=9.6, 2.8 Hz, 1H), 6.66 (d, J=9.6 Hz, 1H), 4.65 (s, 2H), 4.25 (q, J=7.2 Hz, 2H), 3.72 (s, 2H), 3.68 (t, J=7.2 Hz, 3H), 3.24-3.15 (m, 2H), 1.91-1.79 (m, 2H), 1.73-1.62 (m, 2H), 1.38-1.21 (m, 14l H); LC/MS (ESI, m/z): [(M+1)]+=496.2.

### (S)-2-(5-(N-(11-aminoundecyl)-1-(isoquinolin-4-yl) piperidine-3-carboxamido)-2-oxopyridin-1 (2H)-yl) acetic acid (Intermediate AE)

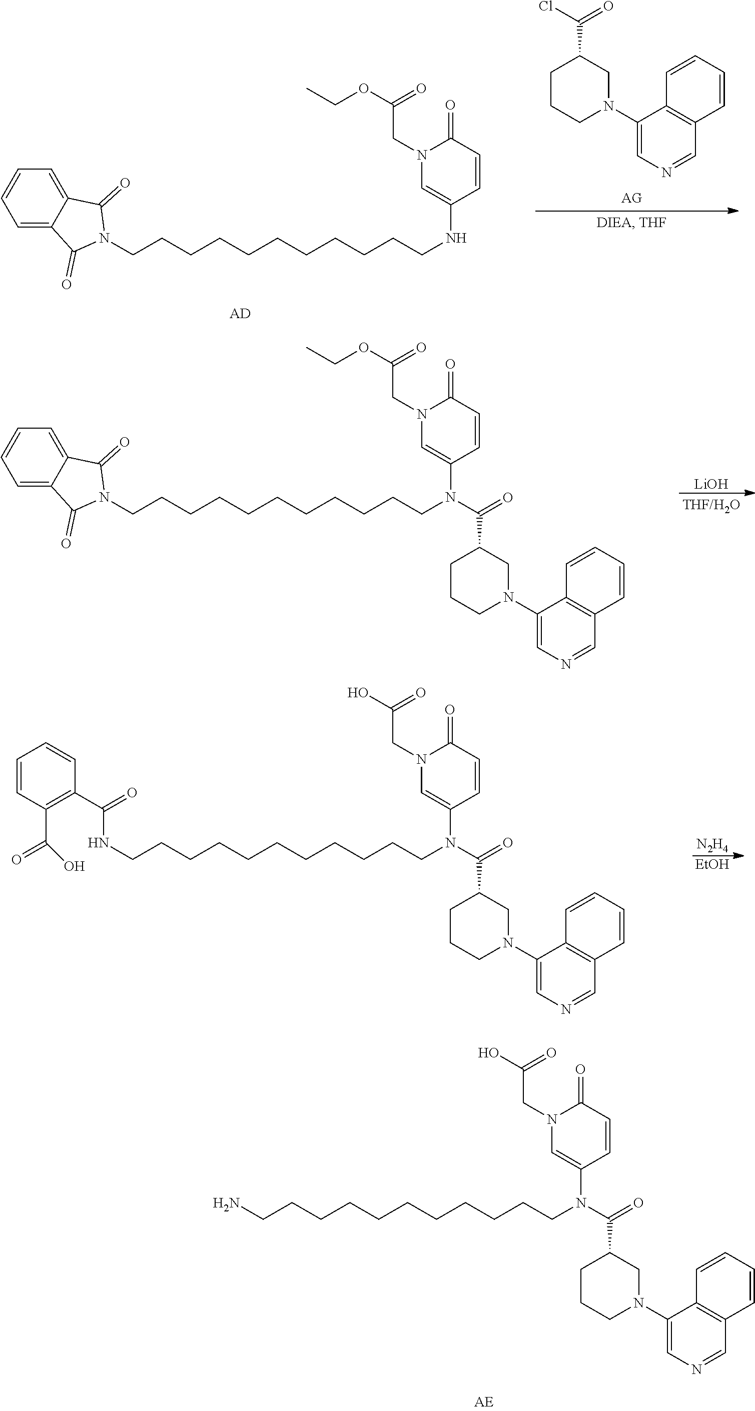

Step 1-Ethyl(S)-2-(5-(N-(11-(1,3-dioxoisoindolin-2-yl) undecyl)-1-(isoquinolin-4-yl) piperidine-3-carboxamido)-2-oxopyridin-1 (2H)-yl)acetate. To a stirred solution of ethyl 2-(5-((11-(1,3-dioxoisoindolin-2-yl) undecyl)amino)-2-oxopyridin-1 (2H)-yl)acetate hydrochloride (2.0 g, 4.0 mmol, Intermediate AD) in THF (15 mL) was added DIEA (1.56 g, 12.11 mmol) at 0° C. under air atmosphere.

The resulting mixture was then stirred for additional 30 min at rt. Next, (S)-1-(isoquinolin-4-yl) piperidine-3-carbonyl chloride (3.33 g, 12.1 mmol, Intermediate AG) was added in THF (5 mL) dropwise at 0° C. The resulting mixture was then stirred for additional 1 hr at rt. On completion, the reaction mixture was concentrated under reduced pressure. The residue was purified by silica gel column chromatography, eluted with CH₂Cl2/MeOH (10:1), to afford the title compound (1.3 g, 33%) as a dark green solid. 1H NMR (400 MHz, CDC13) δ 8.97 (s, 1H), 8.19 (s, 1H), 8.01-7.89 (m, 2H), 7.89-7.81 (m, 2H), 7.76-7.56 (m, 4H), 7.26-7.14 (m, 2H), 6.68 (d, J=9.6 Hz, 1H), 4.68-4.50 (m, 1H), 4.31-4.04 (m, 2H), 3.75-3.45 (m, 5H), 3.44-3.26 (m, 2H), 3.22-3.01 (m, 1H), 3.04-2.73 (m, 2H), 1.96-1.76 (m, 4H), 1.74-1.60 (m, 3H), 1.58-1.46 (m, 2H), 1.37-1.16 (m, 16l H); LC/MS (ESI, m/z): [(M+1)]+=734.3.

Step 2-(S)-2-((11-(N-(1-(carboxymethyl)-6-oxo-1,6-dihydropyridin-3-yl)-1-(isoquinolin-4-y1) piperidine-3-carboxamido) undecyl) carbamoyl)benzoic acid. To a stirred solution of ethyl(S)-2-(5-(N-(11-(1,3-dioxoisoindolin-2-yl) undecyl)-1-(isoquinolin-4-yl) piperidine-3-carboxamido)-2-oxopyridin-1 (2H)-yl)acetate (1.3 g, 1.77 mmol) and LiOH (170 mg, 7.08 mmol) in THF (10 mL) was added H₂O (10 mL) at rt under air atmosphere. The resulting mixture was stirred for 1 h at rt. On completion, the mixture was concentrated under reduced pressure. The mixture was acidified to pH 5 with HCl (aq.). The precipitated solids were collected by filtration and washed with water (3×5 mL), then dried under reduced pressure to give the title compound (1.2 g, 74% yield) as a dark green solid. 1H NMR (400 MHZ, DMSO-d₆)δ 8.98 (s, 1H), 8.41-8.27 (m, 1H), 8.13 (s, 1H), 8.08 (d, J=8.0 Hz, 1H), 7.87 (d, J=2.8 Hz, 1H), 7.81-7.63 (m, 4H), 7.58-7.44 (m, 2H), 7.39 (dd, J=7.2, 1.6 Hz, 1H), 6.51 (d, J=9.6 Hz, 1H), 4.79-4.45 (m, 3H), 3.28 (t, J=13.2 Hz, 2H), 3.20-3.07 (m, 3H), 2.97-2.73 (m, 3H), 1.82-1.74 (m, 1H), 1.72-1.55 (m, 1H), 1.53-1.36 (m, 4H), 1.31-1.14 (m, 16l H); LC/MS (ESI, m/z): [(M+1)]+=724.3.

Step 3-(S)-2-(5-(N-(11-aminoundecyl)-1-(isoquinolin-4-yl) piperidine-3-carboxamido)-2-oxopyridin-1 (2H)-yl) acetic acid. To a stirred solution of(S)-2-((11-(N-(1-(carboxymethyl)-6-oxo-1,6-dihydropyridin-3-yl)-1-(isoquinolin-4-yl) piperidine-3-carboxamido) undecyl) carbamoyl)benzoic acid (1.1 g, 1.5 mmol) in EtOH (15 mL) was added NH₂NH₂.H₂O (2.21 mL, 45.6 mmol) dropwise at rt under air atmosphere. The resulting mixture was stirred for 16 hr at 80° C. On completion, the mixture was concentrated under reduced pressure. The residue was purified by reverse phase flash chromatography (Column: WelFlash™ C18-I, 20-40 um, 330 g; Eluent A: Water (plus 10 mmol/L NH₄HCO3); Eluent B: ACN; Gradient: 25%-55% B in 25 min; Flow rate: 80 mL/min; Detector: 220 nm; desired fractions were collected at 40% B) and concentrated under reduced pressure to afford the title compound (500 mg, 57%) as a light brown solid. 1H NMR (300 MHz, DMSO-d₆) δ 8.98 (s, 1H), 8.46 (s, 3H), 8.12 (s, 1H), 8.12-8.02 (m, 1H), 7.80-7.59 (m, 4H), 7.41 (d, J=9.2 Hz, 1H), 6.43 (d, J=9.6 Hz, 1H), 4.49-4.35 (m, 2H), 3.38-3.21 (m, 3H), 2.96-2.65 (m, 5H), 1.95-1.84 (m, 1H), 1.82-1.49 (m, 4H), 1.47-1.37 (m, 2H), 1.33-1.13 (m, 16l H); LC/MS (ESI, m/z): [(M-1)]-=574.2.

### 2-(5-((S)—N-(11-((5S,8S,10aR)-5-amino-8-(((S)-5-amino-1-(benzhydrylamino)-1,5-dioxopentan-2-yl) carbamoyl)-6-oxodecahydropyrrolo[1,2-a][1,5] diazocine-3-carboxamido) undecyl)-1-(isoquinolin-4-yl) piperidine-3-carboxamido)-2-oxopyridin-1 (2H)-yl) acetic acid (Intermediate AF)

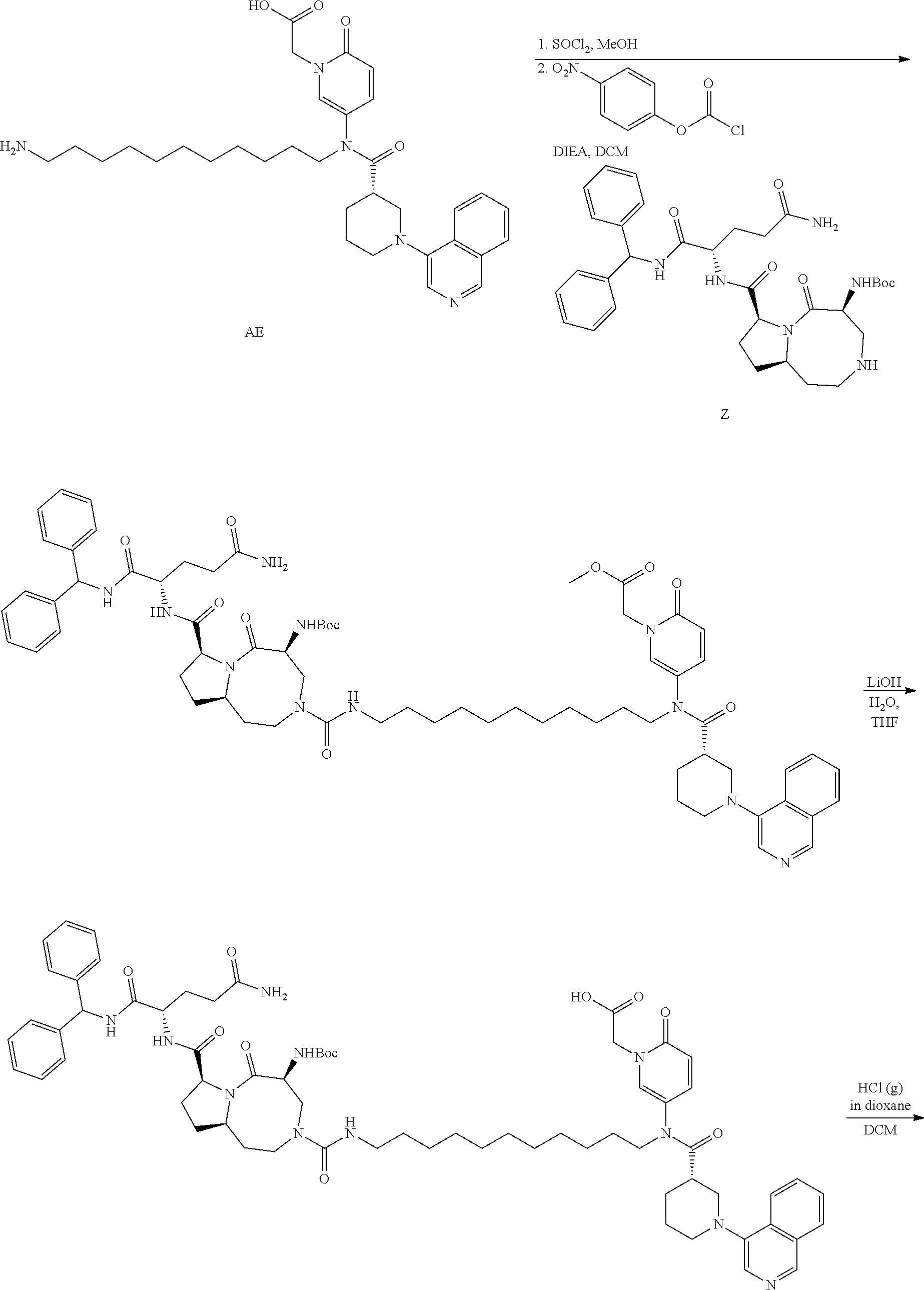

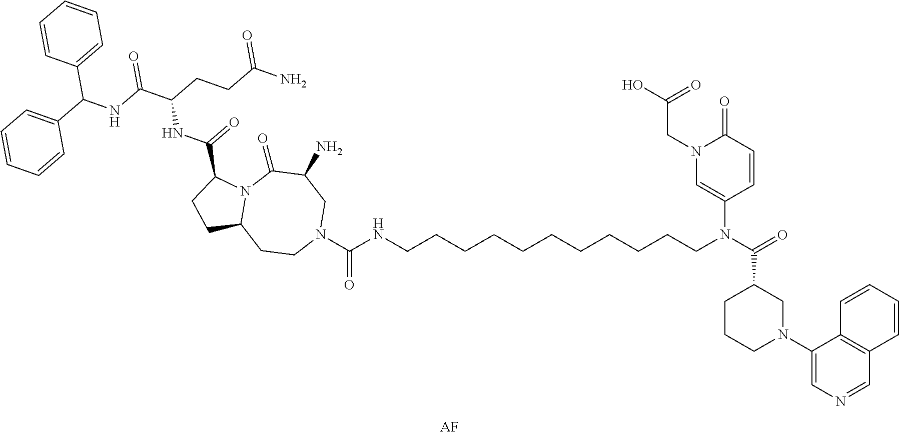 [SMILES: NC(=O)CC[C@H](NC(=O)[C@@H]1CC[C@@H]2CCN(C(=O)NCCCCCCCCCCCN(C(=O)[C@H]3CCCN(c4cncc5ccccc45)C3)c3ccc(=O)n(CC(=O)O)c3)C[C@H](N)C(=O)N21)C(=O)NC(c1ccccc1)c1ccccc1 ; "AF"]

Step 1-Methyl 2-(5-((S)—N-(11-((5S,8S,10aR)-8-(((S)-5-amino-1-(benzhydrylamino)-1,5-dioxopentan-2-yl) carbamoyl)-5-((tert-butoxycarbonyl)amino)-6-oxodecahydropyrrolo[1,2-a] [1,5] diazocine-3-carboxamido) undecyl)-1-(isoquinolin-4-yl) piperidine-3-carboxamido)-2-oxopyridin-1 (2H)-yl)acetate. To a stirred solution of(S)-2-(5-(N-(11-aminoundecyl)-1-(isoquinolin-4-yl) piperidine-3-carboxamido)-2-oxopyridin-1 (2H)-yl) acetic acid (500 mg, 0.87 mmol, Intermediate AE) in MeOH (5 mL) was added SOCI2 (310 mg, 2.60 mmol) dropwise at rt under air atmosphere. The resulting mixture was stirred for 1 hr at 70° C. Then the mixture was concentrated under reduced pressure. Next, 4-nitrophenyl carbonochloridate (192 mg, 0.95 mmol) and TEA (84 mg, 0.83 mmol) in DCM (5 mL) were added at 0° C. to the reaction mixture. Then the mixture was stirred for additional 20 min at 0° C. Next, tert-butyl ((5S,8S,10aR)-8-(((S)-5-amino-1-(benzhydrylamino)-1,5-dioxopentan-2-yl) carbamoyl)-6-oxodecahydropyrrolo[1,2-a][1,5] diazocin-5-yl) carbamate (539 mg, 0.87 mmol, Intermediate Z) was added in portions at rt and the mixture was stirred for additional 16 hr at rt. On completion, the mixture was concentrated under reduced pressure. The crude product was purified by reverse phase flash chromatography (column: WelFlash™ C18-I, 20-40 um, 330 g; Eluent A: Water (plus 10 mmol/L NH₄HCO3); Eluent B: ACN; Gradient: 45%-95% B in 25 min; Flow rate: 80 mL/min; Detector: 220 nm; desired fractions were collected at 81% B) and concentrated under reduced pressure to afford the title compound (400 mg, 35% yield) as a white solid. 1H NMR (400 MHZ, DMSO-d₆) δ 8.99 (s, 1H), 8.73 (d, J=8.4 Hz, 1H), 8.21 (d, J=7.6 Hz, 1H), 8.14 (s, 1H), 8.08 (d, J=8.0 Hz, 1H), 7.92 (s, 1H), 7.81-7.75 (m, 1H), 7.73-7.63 (m, 1H), 7.60-7.52 (m, 1H), 7.40-7.20 (m, 11l H), 6.75 (s, 1H), 6.54 (d, J=10.0 Hz, 1H), 6.50-6.40 (m, 2H), 6.10 (d, J=8.4 Hz, 1H), 4.81-4.62 (m, 3H), 4.41 (t, J=8.8 Hz, 1H), 4.37-4.22 (m, 2H), 4.08-3.95 (m, 2H), 3.84-3.74 (m, 2H), 3.73-3.52 (m, 4H), 3.35 (s, 3H), 3.31-3.23 (m, 2H), 3.20-2.96 (m, 2H), 2.94-2.74 (m, 3H), 2.20-2.05 (m, 3H), 1.97-1.85 (m, 3H), 1.85-1.72 (m, 2H), 1.70-1.56 (m, 2H), 1.49-1.39 (s, 13l H), 1.29-1.15 (m, 16l H); LC/MS (ESI, m/z): [(M+1)]+=1236.8.

Step 2-2-(5-((S)—N-(11-((5S,8S,10aR)-8-(((S)-5-amino-1-(benzhydrylamino)-1,5-dioxopentan-2-yl) carbamoyl)-5-((tert-butoxycarbonyl)amino)-6-oxodecahydropyrrolo[1,2-a] [1,5] diazocine-3-carboxamido) undecyl)-1-(isoquinolin-4-yl) piperidine-3-carboxamido)-2-oxopyridin-1 (2H)-yl) acetic acid. A stirred solution of methyl 2-(5-((S)—N-(11-((5S,8S,10aR)-8-(((S)-5-amino-1-(benzhydrylamino)-1,5-dioxopentan-2-yl) carbamoyl)-5-((tert-butoxycarbonyl)amino)-6-oxodecahydropyrrolo[1,2-a][1,5] diazocine-3-carboxamido) undecyl)-1-(isoquinolin-4-yl) piperidine-3-carboxamido)-2-oxopyridin-1 (2H)-yl)acetate (200 mg, 0.16 mmol) and LiOH (16 mg, 0.65 mmol) in THF (2 mL) and H₂O (2 mL) was stirred under air atmosphere for 1 hr at rt. On completion, the reaction mixture was concentrated under reduced pressure. The crude product was purified by reverse phase flash chromatography (column: WclFlash™ C18-I, 20-40 um, 120 g; Eluent A: Water (plus 10 mmol/L NH₄HCO3); Eluent B: ACN; Gradient: 25%-55% B in 25 min; Flow rate: 60 mL/min; Detector: 220 nm; desired fractions were collected at 40% B) and concentrated under reduced pressure to give the title compound (180 mg, 90% yield) as a yellow solid. LC/MS (ESI, m/z): [(M+1)]+=1222.5.

Step 3-2-(5-((S)—N-(11-((5S,8S,10aR)-5-amino-8-(((S)-5-amino-1-(benzhydrylamino)-1,5-dioxopentan-2-yl) carbamoyl)-6-oxodecahydropyrrolo[1,2-a][1,5] diazocine-3-carboxamido) undecyl)-1-(isoquinolin-4-yl) piperidine-3-carboxamido)-2-oxopyridin-1 (2H)-yl) acetic acid hydrochloride. To a stirred mixture of 2-(5-((S)—N-(11-((5S,8S,10aR)-8-(((S)-5-amino-1-(benzhydrylamino)-1,5-dioxopentan-2-yl) carbamoyl)-5-((tert-butoxycarbonyl)amino)-6-oxodecahydropyrrolo[1,2-a][1,5] diazocine-3-carboxamido) undecyl)-1-(isoquinolin-4-yl) piperidine-3-carboxamido)-2-oxopyridin-1 (2H)-yl) acetic acid (180 mg, 0.15 mmol) in DCM (2 mL) was added HCl (gas) in 1,4-dioxane (2 mL) dropwise at rt. The resulting mixture was stirred for 1 hr at rt. On completion, the mixture was concentrated under reduced pressure to afford the title compound (160 mg, 96% yield) as a yellow solid. 1H NMR (400 MHZ, DMSO-d₆)δ 13.13 (s, 1H), 9.42 (s, 1H), 8.85 (d, J=8.4 Hz, 1H), 8.42 (d, J=8.4 Hz, 1H), 8.38-8.26 (m, 3H), 8.19 (s, 1H), 8.08-8.00 (m, 1H), 7.99-7.89 (m, 2H), 7.57-7.49 (m, 1H), 7.40-7.20 (m, 10l H), 6.78 (s, 1H), 6.61 (s, 1H), 6.53 (d, J=9.6 Hz, 1H), 6.10 (d, J=8.4 Hz, 1H), 4.81-4.55 (m, 2H), 4.46 (t, J=8.8 Hz, 1H), 4.43-4.33 (m, 1H), 4.25 (d, J=13.8 Hz, 1H), 4.20-4.10 (m, 1H), 4.11-3.99 (m, 1H), 3.87-3.60 (m, 13l H), 3.45-3.33 (m, 2H), 3.26-3.07 (m, 1H), 3.05-2.80 (m, 4H), 2.26-2.04 (m, 2H), 1.99-1.85 (m, 3H), 1.84-1.58 (m, 3H), 1.56-1.35 (m, 4H), 1.32-1.16 (m, 14l H); LC/MS (ESI, m/z): [(M+1)]+=1122.5.

### (S)-1-(isoquinolin-4-yl) piperidine-3-carbonyl chloride (Intermediate AG)

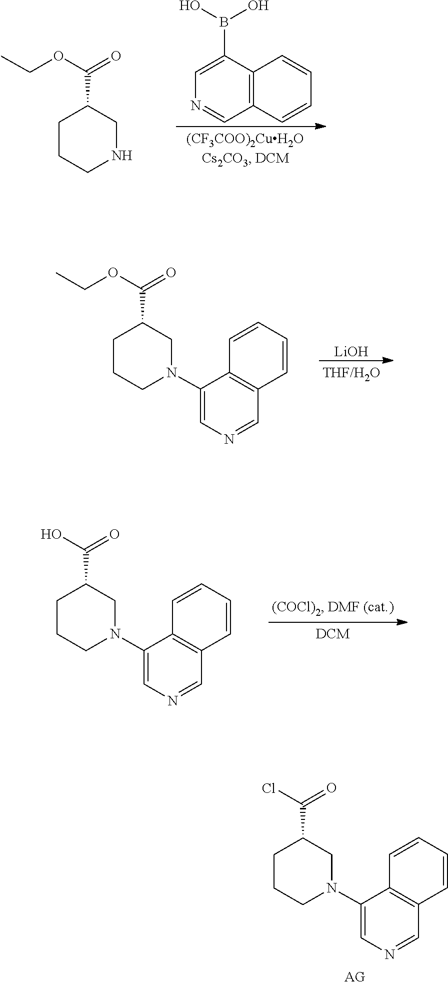

Step 1-Ethyl(S)-1-(isoquinolin-4-yl) piperidine-3-carboxylate. To a stirred solution of ethyl(S)-piperidine-3-carboxylate (30.00 g, 190.8 mmol), isoquinolin-4-ylboronic acid (49.51 g, 286.24 mmol) and Cs₂CO₃ (186.52 g, 572.47 mmol) in DCM (600 mL) was added Cu (OTf)₂ (138.03 g, 381.7 mmol) at r t under air atmosphere. The resulting mixture was stirred for 16 h at rt under air atmosphere. On completio n, the mixture was filtered, and the filter cake was washed with DCM (3×200 mL). The filtrate was concentrated under reduced pressure. The residue was purified by silica gel column chromatography, eluted with petroleum ether/EtOAc (38%), and concentrated under reduced pressure to afford the title compound (12 g, 22% yield) as a purple solid. 1H NMR (400 MHZ, DMSO-d₆) δ 9.02 (s, 1H), 8.20 (s, 1H), 8.13-8.05 (m, 2H), 7.86-7.74 (m, 1H), 7.74-7.61 (m, 1H), 4.12 (q, J=7.2 Hz, 2H), 3.42-3.39 (m, 1H), 3.25-3.16 (m, 1H), 3.13-3.02 (m, 1H), 2.97-2.82 (m, 2H), 2.05-1.95 (m, 1H), 1.95-1.87 (m, 1H), 1.87-1.62 (m, 2H), 1.1 9 (t, J=7.2 Hz, 3H); LC/MS (ESI, m/z): [(M+1)]+=385.2.

Step 2-(S)-1-(isoquinolin-4-yl) piperidine-3-carboxylic acid. To a stirred solution of ethyl(S)-1-(isoquinolin-4-yl) piperidine-3-carboxylate (12 g, 42 mmol) in THF (100 mL) and H₂O (100 mL) was a dded LiOH (5.05 g, 211 mmol) in portions at rt under nitrogen atmosphere. The resulting mixture was the n stirred for 16 hr at rt under nitrogen atmosphere. On completion, the mixture was concentrated under va cuum. The residue was purified by reverse phase Flash chromatography (Column: WelFlash™ C18-I, 2 0-40 um, 330 g; Eluent A: Water (10 mmol/L NH₄HCO3); Eluent B: acetonitrile; Gradient: 5%-30% B in 40 min; Flow rate: 80 mL/min; Detector: 220/254 nm; desired fractions were collected at 20% B) and con centrated under reduced pressure to afford the title compound (9 g, 83% yield) as a yellow solid. LC/MS (ESI, m/z): [(M+1)]+=257.0.

Step 3-(S)-1-(isoquinolin-4-yl) piperidine-3-carbonyl chloride. To a stirred solution of(S)-1-(isoquinolin-4-yl) piperidine-3-carboxylic acid (5 g, 20 mmol) and oxalic dichloride (4.95 g, 39.0 mmol) in DCM (50 mL) was added DMF (0.148 mL, 1.95 mmol) dropwise at rt under nitrogen atmosphere. The resulting mixture was stirred for 1 hr at rt under nitrogen atmosphere. On completion, the mixture was concentrated under vacuum to give the title compound (4 g, 75% yield) as a yellow solid. LC/MS (ESI, m/z): [(M+MeOH)]+=271.2.

### Ethyl 2-(5-((14-azido-3,6,9,12-tetraoxatetradecyl)amino)-2-oxopyridin-1 (2H)-yl)acetate (Intermediate AH)

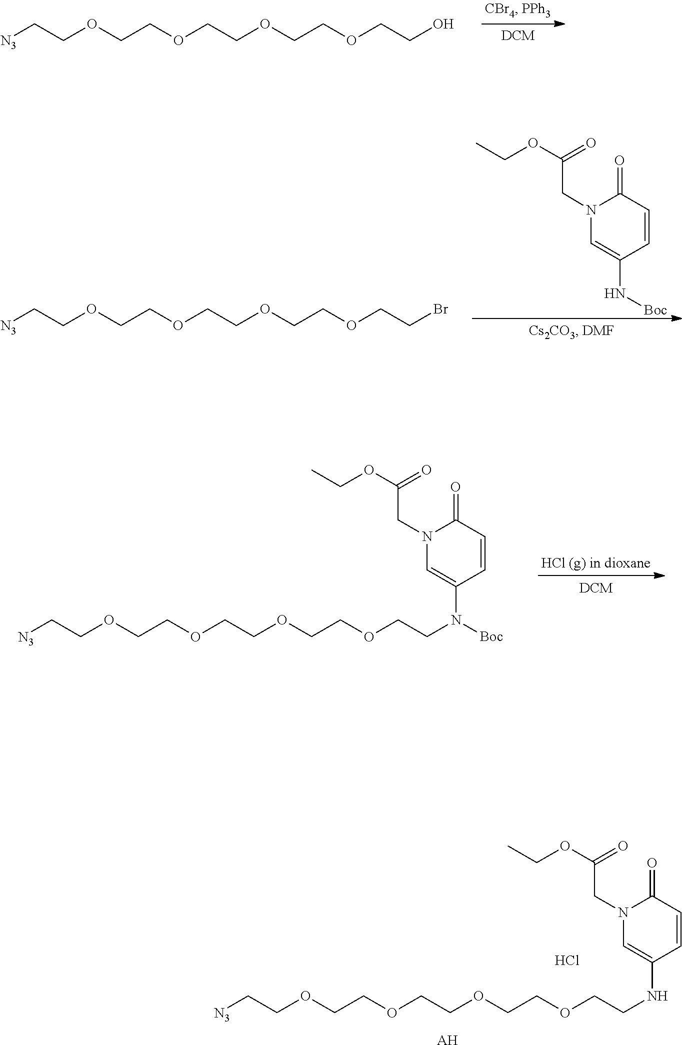

Step 1-1-Azido-14-bromo-3,6,9,12-tetraoxatetradecane. To a stirred solution of 14-azido-3,6,9,12-tetraoxatetradecan-1-ol (3.0 g, 11 mmol, CAS #86770-68-5) and CBr4 (7.56 g, 22.79 mmol) in DCM (45 mL) was added PPh3 (5.98 g, 22.79 mmol) at 0° C. under nitrogen atmosphere. The resulting mixture was then stirred for 1 hr at rt under nitrogen atmosphere. On completion, the mixture was concentrated under vacuum. The residue was purified by silica gel column chromatography, cluted with PE/EA (1:1) to afford the title compound (3.1 g, 83% yield) as a colorless oil. 1H NMR (300 MHZ, CDCl₃) δ 3.77 (t, J=6.4 Hz, 2H), 3.66-3.60 (m, 14l H), 3.43 (t, J=6.4 Hz, 2H), 3.34 (t, J=5.2 Hz, 2H).

Step 2-Ethyl 2-(5-((14-azido-3,6,9,12-tetraoxatetradecyl)(tert-butoxycarbonyl)amino)-2-oxopyridin-1 (2H)-yl)acetate. To a stirred solution of 1-azido-14-bromo-3,6,9,12-tetraoxatetradecane (3.1 g, 9.5 mmol) and ethyl 2-(5-((tert-butoxycarbonyl)amino)-2-oxopyridin-1 (2H)-yl)acetate (2.25 g, 7.60 mmol, synthesized via Steps 1-2 of Intermediate A) in DMF (45 mL) was added Cs₂CO₃ (9.29 g, 28.5 mmol) in portions at rt under nitrogen atmosphere. The resulting mixture was stirred for 16 hr at 50° C. under nitrogen atmosphere. The residue was purified by reverse phase flash chromatography (Column: WelFlash™ C18-I, 20-40 um, 120 g; Eluent A: Water (10 mmol/L NH₄HCO₃); Eluent B: acetonitrile; Gradient: 25%-55% B in 40 min; Flow rate: 80 mL/min; Detector: 220/254 nm; desired fractions were collected at 40% B) and concentrated under reduced pressure to afford the title compound (1.1 g, 21% yield) as a yellow oil. ¹H NMR (400 MHZ, DMSO-d₆) δ 7.69 (d, J=2.8 Hz, 1H), 7.41 (dd, J=9.6, 2.8 Hz, 1H), 6.37 (d, J=9.6 Hz, 1H), 4.65 (s, 2H), 4.14 (q, J=7.2 Hz, 2H), 3.67-3.44 (m, 18l H), 3.44-3.34 (m, 2H), 1.37 (s, 9H), 1.21 (t, J=7.2 Hz, 3H); LC/MS (ESI, m/z): [(M+1)]+=542.2.

Step 3-Ethyl 2-(5-((14-azido-3,6,9,12-tetraoxatetradecyl)amino)-2-oxopyridin-1 (2H)-yl)acetate hydrochloride. To a stirred solution of ethyl 2-(5-((14-azido-3,6,9,12-tetraoxatetradecyl)(tert-butoxycarbonyl)amino)-2-oxopyridin-1 (2H)-yl)acetate (1.1 g, 2.0 mmol) in DCM (10 mL) was added HCl (gas) in 1,4-dioxane (10 mL) at rt. The resulting mixture was then stirred for 1 hr at rt. On completion, the mixture was concentrated under vacuum to give the title compound (1.1 g) as a yellow oil. 1H NMR (300 MHz, DMSO-d₆) δ 7.80 (d, J=3.2 Hz, 1H), 7.63 (d, J=9.6, 3.2 Hz, 1H), 6.54 (d, J=9.6 Hz, 1H), 4.72 (s, 2H), 4.16 (q, J=7.2 Hz, 2H), 3.74-3.47 (m, 16l H), 3.44-3.35 (m, 2H), 3.36-3.26 (m, 2H), 1.22 (t, J=7.2 Hz, 3H). LC/MS (ESI, m/z): [(M+1)]+=442.2.

### Ethyl(S)-2-(5-(N-(14-amino-3,6,9,12-tetraoxatetradecyl)-1-(isoquinolin-4-yl) piperidine-3-carboxamido)-2-oxopyridin-1 (2H)-yl)acetate (Intermediate AI)

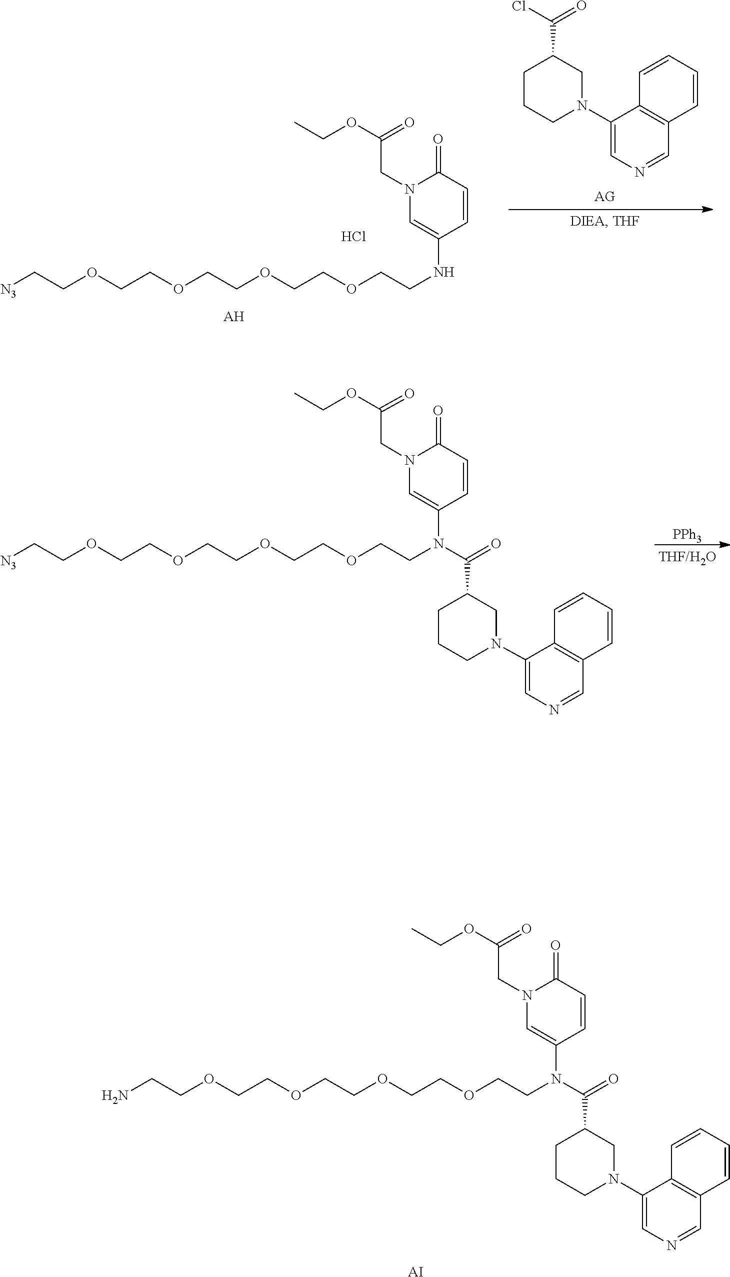

Step 1-Ethyl(S)-2-(5-(N-(14-azido-3,6,9,12-tetraoxatetradecyl)-1-(isoquinolin-4-yl) piperidine-3-carboxamido)-2-oxopyridin-1 (2H)-yl)acetate. To a solution of ethyl 2-(5-((14-azido-3,6,9,12-tetraoxatetradecyl)amino)-2-oxopyridin-1 (2H)-yl)acetate hydrochloride (1.0 g, 2.3 mmol, Intermediate AH) in THF (15 mL) was added DIEA (0.88 g, 6.80 mmol) at 0° C. and the mixture was stirred for 30 min at 0° C. under nitrogen atmosphere. Next, (S)-1-(isoquinolin-4-yl) piperidine-3-carbonyl chloride (0.93 g, 3.4 mmol, Intermediate AG) in DCM (2 mL) was added dropwise at 0° C. to the mixture. The resulting mixture was then stirred for 1 hr at rt under nitrogen atmosphere. On completion, the mixture was concentrated under vacuum. The crude product was purified by reverse phase flash chromatography (Column: WelFlash™ C18-I, 20-40 um, 120 g; Eluent A: Water (10 mmol/L NH₄HCO3); Eluent B: acetonitrile; Gradient: 25%-55% B in 40 min; Flow rate: 80 mL/min; Detector: 220/254 nm; desired fractions were collected at 50% B) and concentrated under reduced pressure to afford the title compound (770 mg, 50% yield) as a yellow solid. LC/MS (ESI, m/z): [(M+1)]+=680.3.

Step 2-Ethyl(S)-2-(5-(N-(14-amino-3,6,9,12-tetraoxatetradecyl)-1-(isoquinolin-4-yl) piperidine-3-carboxamido)-2-oxopyridin-1 (2H)-yl)acetate. To a stirred solution of ethyl(S)-2-(5-(N-(14-azido-3,6,9,12-tetraoxatetradecyl)-1-(isoquinolin-4-yl) piperidine-3-carboxamido)-2-oxopyridin-1 (2H)-yl)acetate (770 mg, 1.13 mmol) in THF (8 mL) and H₂O (2 mL) was added PPh3 (594 mg, 2.27 mmol) at rt under nitrogen atmosphere. The resulting mixture was then stirred for 16 h at 50° C. under nitrogen atmosphere. On completion, the mixture was concentrated under vacuum. The residue was purified by reverse phase flash chromatography (Column: WelFlash™ C18-I, 20-40 um, 330 g; Eluent A: Water (10 mmol/L NH₄HCO3); Eluent B: acetonitrile; Gradient: 15%-45% B in 40 min; Flow rate: 80 mL/min; Detector: 220/254 nm; desired fractions were collected at 32% B) and concentrated under reduced pressure to afford the title compound (320 mg, 43% yield) as a yellow oil. LC/MS (ESI, m/z): [(M+1)]+=654.4.

2-(5-((S)—N-(1-((5S,8S,10aR)-5-amino-8-(((S)-5-amino-1-(benzhydrylamino)-1,5-dioxopentan-2-yl) carbamoyl)-6-oxooctahydropyrrolo[1,2-a] [1,5l] diazocin-3 (4l H)-yl)-1-oxo-5,8,11,14-tetraoxa-2-azahexadecan-16-yl)-1-(isoquinolin-4-yl) piperidine-3-carboxamido)-2-oxopyridin-1 (2H)-yl) acetic acid (Intermediate AJ)

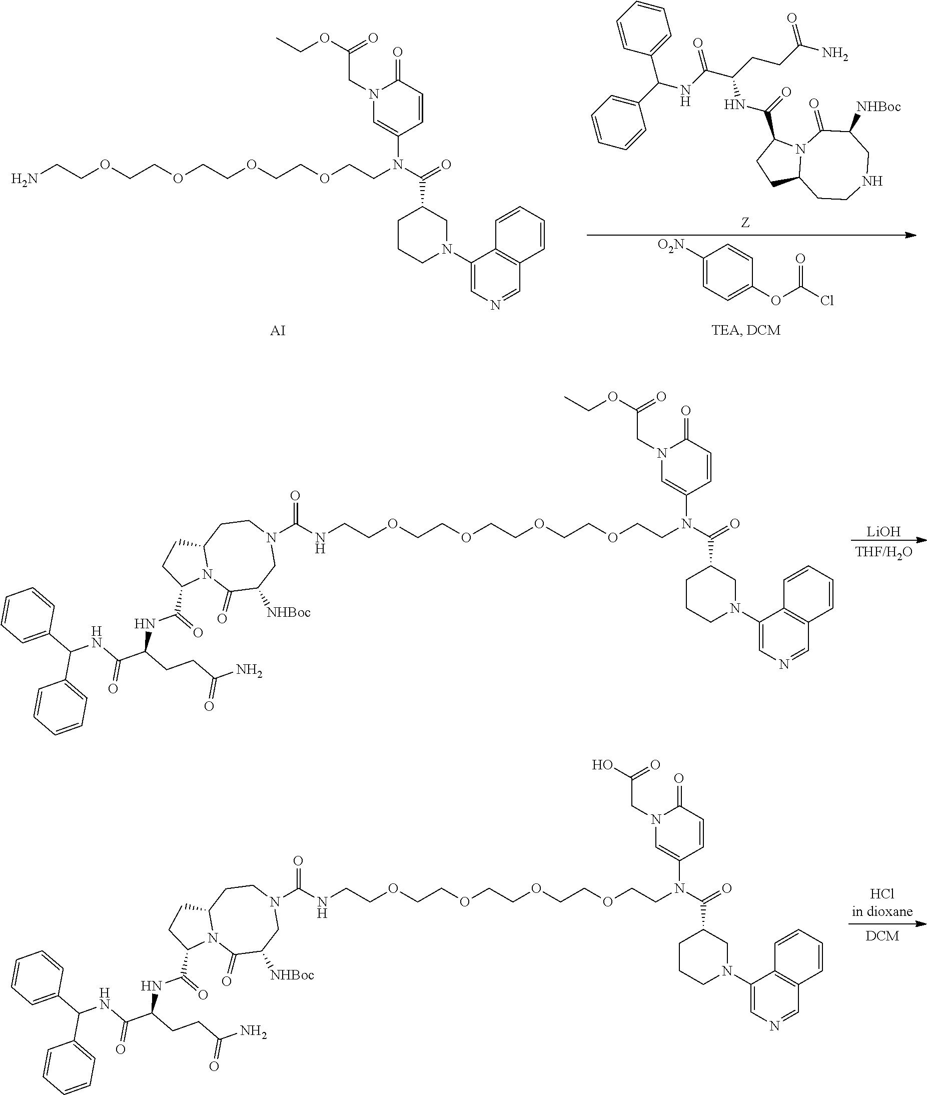

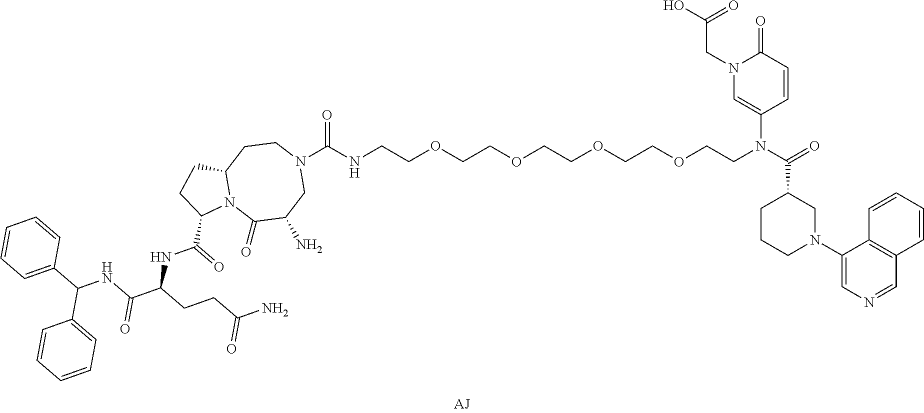 [SMILES: NC(=O)CC[C@H](NC(=O)[C@@H]1CC[C@@H]2CCN(C(=O)NCCOCCOCCOCCOCCN(C(=O)[C@H]3CCCN(c4cncc5ccccc45)C3)c3ccc(=O)n(CC(=O)O)c3)C[C@H](N)C(=O)N21)C(=O)NC(c1ccccc1)c1ccccc1]

Step 1-Ethyl 2-(5-((S)—N-(1-((5S,8S,10aR)-8-(((S)-5-amino-1-(benzhydrylamino)-1,5-dioxopentan-2-yl) carbamoyl)-5-((tert-butoxycarbonyl)amino)-6-oxooctahydropyrrolo[1,2-a] [1,5] diazocin-3 (4//)-yl)-1-oxo-5,8,11,14-tetraoxa-2-azahexadecan-16-yl)-1-(isoquinolin-4-yl) piperidine-3-carboxamido)-2-oxopyridin-1 (2H)-yl)acetate. To a stirred solution of ethyl(S)-2-(5-(N-(14-amino-3,6,9,12-tetraoxatetradecyl)-1-(isoquinolin-4-yl) piperidine-3-carboxamido)-2-oxopyridin-1 (2H)-yl)acetate (150 mg, 0.23 mmol, Intermediate AI) in DCM (2 mL) was added 4-nitrophenyl carbonochloridate (51 mg, 0.25 mmol) and TEA (70 mg, 0.69 mmol). The resulting mixture was stirred for 1 hr at rt under nitrogen atmosphere. To the above mixture was added tert-butyl ((5S,85,10aR)-8-(((S)-5-amino-1-(benzhydrylamino)-1,5-dioxopentan-2-yl) carbamoyl)-6-oxodecahydropyrrolo[1,2-a] [1,5] diazocin-5-yl) carbamate (157 mg, 0.25 mmol, Intermediate Z) in DCM (2 mL). The resulting mixture was then stirred for 16 hr at rt under nitrogen atmosphere. On completion, the mixture was concentrated under vacuum. The crude product was purified by reverse phase flash chromatography (Column: WelFlash™ C18-I, 20-40 um, 120 g; Eluent A: Water (10 mmol/L NH₄HCO3); Eluent B: acetonitrile; Gradient: 25%-55% B in 40 min; Flow rate: 80 mL/min; Detector: 220/254 nm; desired fractions were collected at 37% B) and concentrated under reduced pressure to afford the title compound (160 mg, 53% yield) as a yellow solid. LC/MS (ESI, m/z): [(M+1)]+=1300.6.

Step 2-2-(5-((S)—N-(1-((5S,8S,10aR)-8-(((S)-5-amino-1-(benzhydrylamino)-1,5-dioxopentan-2-yl) carbamoyl)-5-((tert-butoxycarbonyl)amino)-6-oxooctahydropyrrolo[1,2-a] [1,5] diazocin-3 (4H)-yl)-1-oxo-5,8,11,14-tetraoxa-2-azahexadecan-16-yl)-1-(isoquinolin-4-yl) piperidine-3-carboxamido)-2-oxopyridin-1 (2H)-yl) acetic acid. To a stirred solution of ethyl 2-(5-((S)-N-(1-((5S,8S,10aR)-8-(((S)-5-amino-1-(benzhydrylamino)-1,5-dioxopentan-2-yl) carbamoyl)-5-((tert-butoxycarbonyl)amino)-6-oxooctahydropyrrolo[1,2-a][1,5] diazocin-3 (4/I)-yl)-1-oxo-5,8,11, 14-tetraoxa-2-azahexadecan-16-yl)-1-(isoquinolin-4-yl) piperidine-3-carboxamido)-2-oxopyridin-1 (2H)-yl)acetate (166 mg, 0.13 mmol) in THF (2 mL) and H₂O (2 mL) was added LiOH (12 mg, 0.51 mmol) at room temperature. The resulting mixture was stirred for 1 hr at rt. On completion, the mixture was acidified to pH 5 with HCl (aq.). The residue was then purified by reverse phase flash chromatography (Column: WelFlash™ C18-I, 20-40 um, 120 g; Eluent A: Water (10 mmol/L NH₄HCO3); Eluent B: acetonitrile; Gradient: 20%-50% B in 40 min; Flow rate: 45 mL/min; Detector: 220/254 nm; desired fractions were collected at 37% B) and concentrated under reduced pressure to afford the title compound (110 mg, 68% yield) as a yellow solid. 1H NMR (400 MHZ, DMSO-d₆) δ 8.99 (s, 1H), 8.74 (d, J=8.4 Hz, 1H), 8.25 (d, J=8.0 Hz, 1H), 8.13 (s, 1H), 8.11-8.02 (m, 1H), 7.88 (s, 1H), 7.84-7.58 (m, 2H), 7.54 (s, 1H), 7.41-7.18 (m, 11l H), 6.75 (s, 1H), 6.60-6.41 (m, 3H), 6.10 (d, J=8.4 Hz, 1H), 4.78-4.49 (m, 2H), 4.41 (t, J=8.8 Hz, 1H), 4.36-4.29 (m, 1H), 4.29-4.18 (m, 1H), 4.01 (s, 1H), 3.91-3.55 (m, 3H), 3.54-3.38 (m, 20l H), 3.35-3.22 (m, 4H), 3.22-3.08 (m, 2H), 2.97-2.87 (m, 1H), 2.86-2.72 (m, 3H), 2.20-2.10 (m, 2H), 2.01-1.87 (m, 4H), 1.85-1.71 (m, 2H), 1.70-1.56 (m, 2H), 1.56-1.44 (m, 1H), 1.39 (s, 9H). LC/MS (ESI, m/z): [(M+1)]+=1272.6.

Step 3-2-(5-((S)—N-(1-((5S,8S,10aR)-5-amino-8-(((S)-5-amino-1-(benzhydrylamino)-1,5-dioxopentan-2-yl) carbamoyl)-6-oxooctahydropyrrolo[1,2-a][1,5] diazocin-3 (4H)-yl)-1-oxo-5,8,11,14-tetraoxa-2-azahexadecan-16-yl)-1-(isoquinolin-4-yl) piperidine-3-carboxamido)-2-oxopyridin-1 (2H)-yl) acetic acid hydrochloride. To a stirred solution of 2-(5-((S)—N-(1-((5S,8S,10aR)-8-(((S)-5-amino-1-(benzhydrylamino)-1,5-dioxopentan-2-yl) carbamoyl)-5-((tert-butoxycarbonyl)amino)-6-oxooctahydropyrrolo[1,2-a][1,5] diazocin-3 (4H)-yl)-1-oxo-5,8,11,14-tetraoxa-2-azahexadecan-16-yl)-1-(isoquinolin-4-yl) piperidine-3-carboxamido)-2-oxopyridin-1 (2H)-yl) acetic acid (100 mg, 0.08 mmol) in DCM (2 mL) was added HCl (gas) in 1,4-dioxane (1 mL) dropwise at rt. The resulting mixture was stirred for 1 hr at rt. On completion, the resulting mixture was concentrated under vacuum to give the title compound (90 mg, 98% yield) as a yellow solid. LC/MS (ESI, m/z): [(M+1)]+=1172.8.

### Ethyl 2-(5-((14-(1,3-dioxoisoindolin-2-yl)tetradecyl)amino)-2-oxopyridin-1 (2H)-yl)acetate (Intermediate AK)

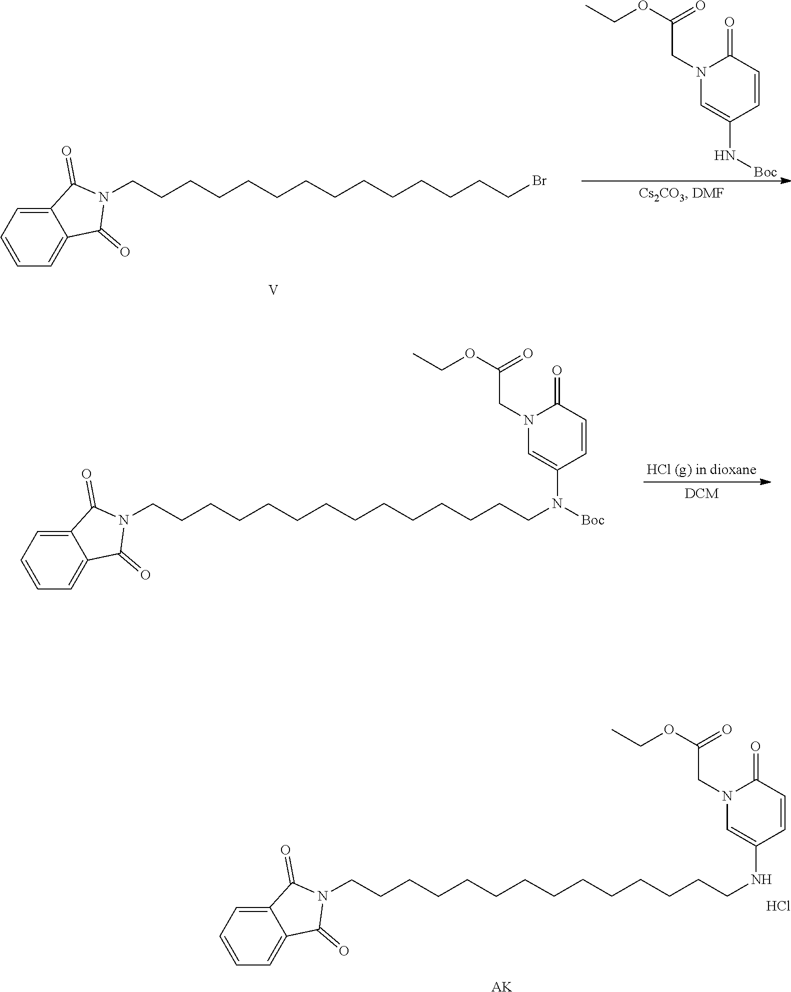

Step 1-Ethyl 2-(5-((tert-butoxycarbonyl)(14-(1,3-dioxoisoindolin-2-yl)tetradecyl)amino)-2-oxopyridin-1 (2H)-yl)acetate. To a stirred solution of 2-(14-bromotetradecyl) isoindoline-1,3-dione (2.57 g, 6.07 mmol, Intermediate V) and Cs₂CO₃ (4.95 g, 15.19 mmol) in DMF (45 mL) was added ethyl 2-(5-((tert-butoxycarbonyl)amino)-2-oxopyridin-1 (2H)-yl)acetate (1.50 g, 5.06 mmol, synthesized via Steps 1-2 of Intermediate A) at rt under nitrogen atmosphere. The resulting mixture was stirred for additional 16 hr at 50° C. On completion, the mixture was extracted with EtOAc (3×100 mL). The combined organic layers were washed with brine (3×200 mL), and dried over anhydrous Na₂SO₄. After filtration, the filtrate was concentrated under reduced pressure. The residue was purified by silica gel column chromatography, eluted with PE/EA (50%), to afford the title compound (2.8 g, 81% yield) as a dark green solid. 1H NMR (400 MHz, CDC13) δ 7.85 (dd, J=5.4, 3.2 Hz, 2H), 7.72 (dd, J=5.4, 3.2 Hz, 2H), 7.24 (s, 1H), 7.14 (s, 1H), 6.59 (d, J=9.6 Hz, 1H), 4.64 (s, 2H), 4.26 (q, J=7.2 Hz, 2H), 3.73-3.64 (m, 3H), 3.53-3.42 (m, 2H), 1.73-1.61 (m, 2H), 1.58-1.50 (m, 1H), 1.44 (s, 9H), 1.38-1.20 (m, 23l H). LC/MS (ESI, m/z): [(M+1)]+=638.3.

Step 2-Ethyl 2-(5-((14-(1,3-dioxoisoindolin-2-yl)tetradecyl)amino)-2-oxopyridin-1 (2H)-yl)acetate hydrochloride. To a stirred solution of ethyl 2-(5-((tert-butoxycarbonyl)(14-(1,3-dioxoisoindolin-2-yl)tetradecyl) amino)-2-oxopyridin-1 (2H)-yl)acetate (2.8 g, 4.39 mmol) in DCM (20 mL) was added HCl (gas) in 1,4-dioxane (20 mL) at rt. The resulting mixture was then stirred for 1 hr at rt. On completion, the mixture was concentrated under reduced pressure to give the title compound (2.5 g, 89% yield) as a yellow solid. 1H NMR (400 MHZ, CDC13)δ 7.89-7.84 (m, 3H), 7.72 (dd, J=5.4, 3.2 Hz, 2H), 7.56 (dd, J=10.0, 2.8 Hz, 1H), 6.66 (d, J=10.0 Hz, 1H), 4.64 (s, 2H), 4.25 (q, J=7.2 Hz, 2H), 3.71-3.65 (m, 2H), 3.27-3.13 (m, 2H), 1.92-1.79 (m, 2H), 1.73-1.61 (m, 2H), 1.40-1.19 (m, 24l H); LC/MS (ESI, m/z): [(M+1)]+=574.3.

### Methyl(S)-2-(5-(N-(14-aminotetradecyl)-1-(isoquinolin-4-yl) piperidine-3-carboxamido)-2-oxopyridin-1 (2H)-yl)acetate (Intermediate AL)

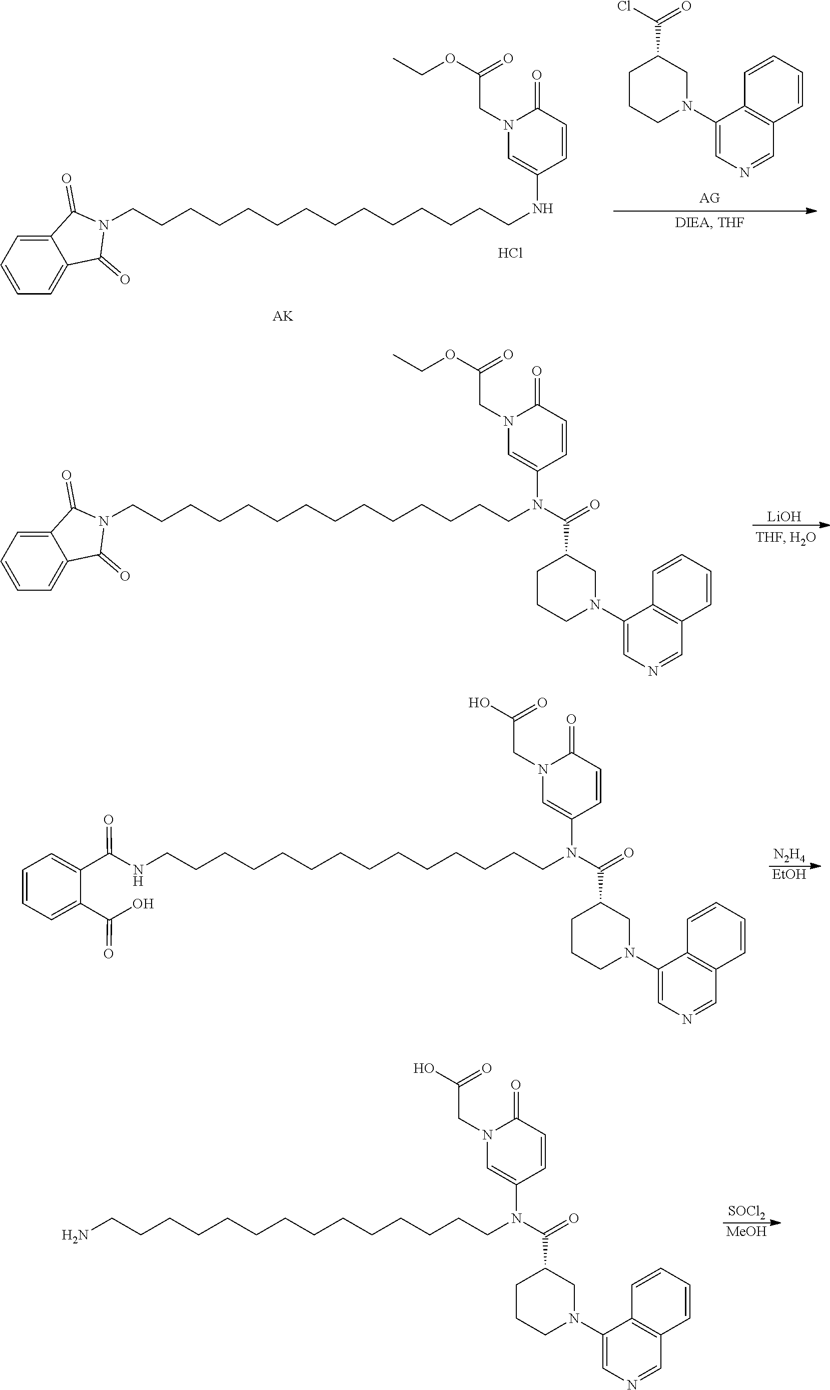

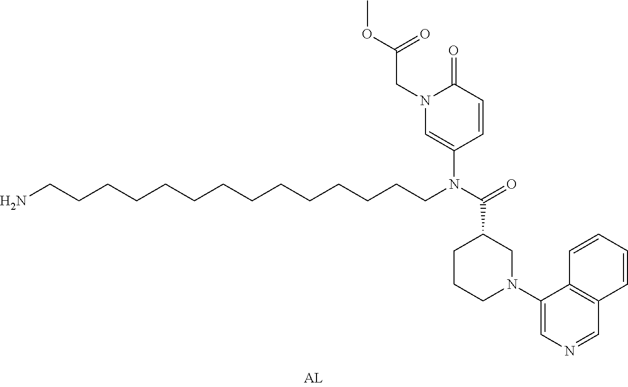 [SMILES: COC(=O)Cn1cc(N(CCCCCCCCCCCCCCN)C(=O)[C@H]2CCCN(c3cncc4ccccc34)C2)ccc1=O]

Step 1-Ethyl(S)-2-(5-(N-(14-(1,3-dioxoisoindolin-2-yl)tetradecyl)-1-(isoquinolin-4-yl) pipcridinc-3-carboxamido)-2-oxopyridin-1 (2H)-yl) acctatc. A stirred solution of ethyl 2-(5-((14-(1,3-dioxoisoindolin-2-yl)tetradecyl) amino)-2-oxopyridin-1 (2H)-yl)acetate hydrochloride (1.30 g, 2.42 mmol, Intermediate AK) and DIEA (1.68 mL, 9.67 mmol) in THF (20 mL) was stirred for 30 min at 0° C. under air atmosphere. Next, (S)-1-(isoquinolin-4-yl) piperidine-3-carbonyl chloride (1.00 g, 3.63 mmol, Intermediate AG) was added at 0° C. to the mixture. The resulting mixture was then stirred for 1 hr at rt. On completion, the mixture was concentrated under vacuum. The crude product was purified by silica gel column chromatography, eluted with CH₂Cl2/MeOH (10:1), to afford the title compound (1.6 g, 85% yield) as a dark green solid. 1H NMR (400 MHZ, DMSO-d₆) δ 8.99 (s, 1H), 8.18-8.02 (m, 2H), 7.98-7.44 (m, 9H), 6.54 (d, J=9.6 Hz, 1H), 4.91-4.51 (m, 2H), 4.23-4.08 (m, 2H), 3.55 (t, J=7.2 Hz, 2H), 3.32-3.18 (m, 3H), 2.96-2.69 (m, 4H), 1.94-1.73 (m, 2H), 1.71-1.52 (m, 4H), 1.47-1.32 (m, 2H), 1.31-1.03 (m, 23l H). LC/MS (ESI, m/z): [(M+1)]+=776.4.

Step 2-(S)-2-((14-(N-(1-(carboxymethyl)-6-oxo-1,6-dihydropyridin-3-yl)-1-(isoquinolin-4-yl) piperidine-3-carboxamido)tetradecyl) carbamoyl)benzoic acid. To a stirred solution of ethyl(S)-2-(5-(N-(14-(1,3-dioxoisoindolin-2-yl)tetradecyl)-1-(isoquinolin-4-yl) piperidine-3-carboxamido)-2-oxopyridin-1 (2H)-yl)acetate (1.6 g, 2.1 mmol) in THF (20 mL) and H₂O (20 mL) was added LiOH (197 mg, 8.25 mmol) at rt. The resulting mixture was stirred for 1 hr at rt. On completion, the mixture was acidified to pH 5 with aq HCl. The mixture was then purified by reversed-phase flash chromatography (column, C18 silica gel; mobile phase, MeCN in Water (10 mmol/L NH₄HCO3), 20% to 60% gradient in 30 min; detector, UV 254 nm) to afford the title compound (1 g, 63% yield) as a light green solid. LC/MS (ESI, m/z): [(M+1)]+=766.4.

Step 3-(S)-2-(5-(N-(14-aminotetradecyl)-1-(isoquinolin-4-yl) piperidine-3-carboxamido)-2-oxopyridin-1 (2H)-yl) acetic acid. To a stirred solution of(S)-2-((14-(N-(1-(carboxymethyl)-6-oxo-1,6-dihydropyridin-3-yl)-1-(isoquinolin-4-yl) piperidine-3-carboxamido)tetradecyl) carbamoyl)benzoic acid (1 g, 1.31 mmol) in EtOH (10 mL) was added hydrazine hydrate (1.96 g, 39.18 mmol) at rt. The resulting mixture was stirred for 16 hr at 80° C. On completion, the mixture was concentrated under vacuum. The crude product was purified by reversed-phase flash chromatography (column, C18 silica gel; mobile phase, MeCN in Water (10 mmol/L NH₄HCO3), 20% to 50% gradient in 30 min; detector, UV 254 nm) to afford the title compound (500 mg, 62% yield) as a light yellow solid. 1H NMR (400 MHZ, DMSO-d₆) δ 8.98 (s, 1H), 8.12 (s, 1H), 8.07 (d, J=8.0 Hz, 1H), 7.96 (s, 1H), 7.79-7.59 (m, 3H), 7.40 (s, 1H), 6.43 (d, J=9.6 Hz, 1H), 4.44-4.34 (m, 2H), 3.41-3.19 (m, 7H), 2.78 (d, J=11.2 Hz, 1H), 2.73-2.65 (m, 1H), 1.96-1.85 (m, 1H), 1.80-1.57 (m, 1H), 1.55-1.47 (m, 1H), 1.45-1.36 (m, 2H), 1.29-1.14 (m, 26l H). LC/MS (ESI, m/z): [(M+1)]+=618.4.

Step 4-Methyl(S)-2-(5-(N-(14-aminotetradecyl)-1-(isoquinolin-4-yl) piperidine-3-carboxamido)-2-oxopyridin-1 (2H)-yl)acetate. To a stirred solution of(S)-2-(5-(N-(14-aminotetradecyl)-1-(isoquinolin-4-yl) piperidine-3-carboxamido)-2-oxopyridin-1 (2H)-yl) acetic acid (500 mg, 0.8 mmol) in MeOH (7 mL) was added SOC12 (385 mg, 3.24 mmol) at rt. The resulting mixture was then stirred for 1 h at 70° C. On completion, the mixture was cooled to rt and concentrated under vacuum to afford the title compound (500 mg, 98% yield) as a yellow solid. LC/MS (ESI, m/z): [(M+1)]+=632.4.

### 2-(5-((S)—N-(14-((5S,8S,10aR)-5-amino-8-(((S)-5-amino-1-(benzhydrylamino)-1,5-dioxopentan-2-yl) carbamoyl)-6-oxodecahydropyrrolo[1,2-a][1,5] diazocine-3-carboxamido)tetradecyl)-1-(isoquinolin-4-yl) piperidine-3-carboxamido)-2-oxopyridin-1 (2H)-yl) acetic acid (Intermediate AM)

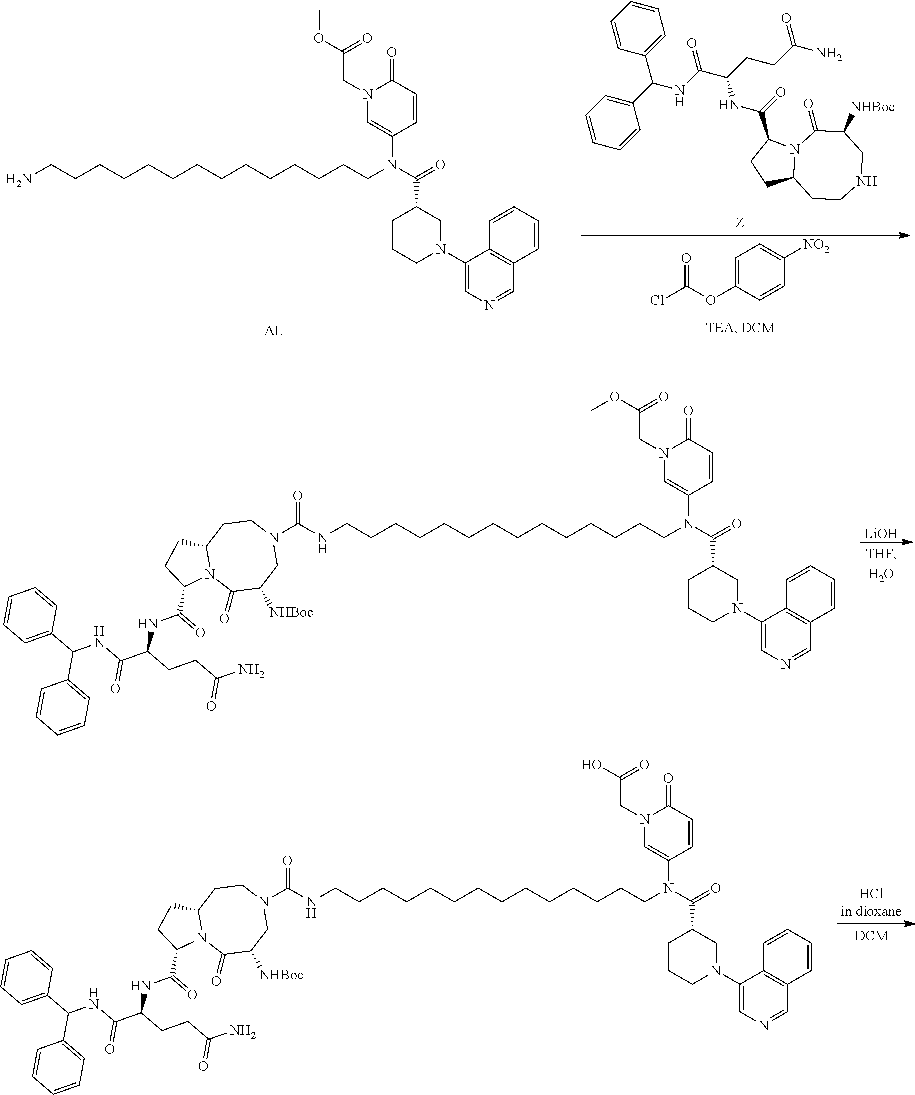

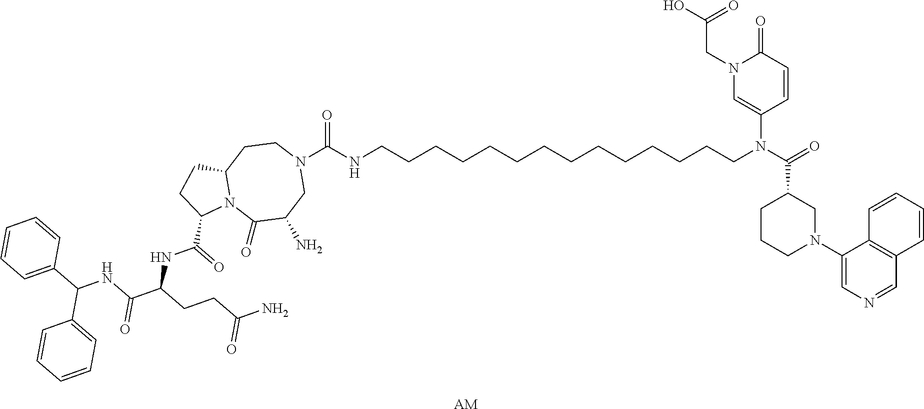

Step 1-Methyl 2-(5-((S)—N-(14-((5S,8S,10aR)-8-(((S)-5-amino-1-(benzhydrylamino)-1,5-dioxopentan-2-yl) carbamoyl)-5-((tert-butoxycarbonyl)amino)-6-oxodecahydropyrrolo[1,2-a] [1,5] diazocinc-3-carboxamido) tctradccyl)-1-(isoquinolin-4-yl) pipcridinc-3-carboxamido)-2-oxopyridin-1 (2H)-yl)acetate. To a stirred solution of methyl(S)-2-(5-(N-(14-aminotetradecyl)-1-(isoquinolin-4-y1) piperidine-3-carboxamido)-2-oxopyridin-1 (2H)-yl)acetate (300 mg, 0.475 mmol, Intermediate AL) in DCM (10 mL) were added TEA (288 mg, 2.850 mmol) and 4-nitrophenyl carbonochloridate (115 mg, 0.570 mmol) at rt. The resulting mixture was stirred for 1 h at rt. Then tert-butyl ((5S,8S,10aR)-8-(((S)-5-amino-1-(benzhydrylamino)-1,5-dioxopentan-2-yl) carbamoyl)-6-oxodecahydropyrrolo[1,2-a][1,5] diazocin-5-y1) carbamate (295 mg, 0.475 mmol, Intermediate Z) was added at rt. The resulting mixture was stirred for 16 hr at 45° C. On completion, the mixture was concentrated under vacuum. The crude product was purified by reversed-phase flash chromatography (column: C18 silica gel; mobile phase, MeCN in Water (0.1% FA), 20% to 50% gradient in 30 min; detector, UV 254 nm) to afford the title compound (330 mg, 54% yield) as an off-white solid. 1H NMR (400 MHZ, DMSO-d₆) δ 12.45 (s, 1H), 8.97 (s, 1H), 8.85 (d, J=8.4 Hz, 1H), 8.73 (d, J=1.6 Hz, 1H), 8.54-8.42 (m, 2H), 8.12 (s, 1H), 8.06 (d, J=8.0 Hz, 1H), 7.94 (dd, J=8.8, 1.6 Hz, 1H), 7.81-7.56 (m, 4H), 7.47 (s, 1H), 7.36-7.18 (m, 10l H), 6.77 (s, 1H), 6.69 (s, 1H), 6.45 (d, J=9.6 Hz, 1H), 6.11 (d, J=8.4 Hz, 1H), 4.82-4.73 (m, 1H), 4.60-4.44 (m, 2H), 4.43-4.31 (m, 1H), 4.26-4.13 (m, 4H), 4.14-3.93 (m, 2H), 3.89-3.79 (m, 1H), 3.29-3.07 (m, 10l H), 3.07-2.88 (m, 1H), 2.85-2.72 (m, 1H), 2.26-2.07 (m, 2H), 2.05-1.81 (m, 2H), 1.80-1.71 (m, 1H), 1.71-1.57 (m, 2H), 1.57-1.45 (m, 3H), 1.44-1.36 (m, 2H), 1.31-1.27 (m, 6H), 1.26-1.10 (m, 26l H). LC/MS (ESI, m/z): [(M+1)]+=1278.7.

Step 2-2-(5-((S)—N-(14-((5S,8S,10aR)-8-(((S)-5-amino-1-(benzhydrylamino)-1,5-dioxopentan-2-yl) carbamoyl)-5-((tert-butoxycarbonyl)amino)-6-oxodecahydropyrrolo[1,2-a] [1,5] diazocine-3-carboxamido)tetradecyl)-1-(isoquinolin-4-yl) piperidine-3-carboxamido)-2-oxopyridin-1 (2H)-yl) acetic acid. To a stirred solution of methyl 2-(5-((S)—N-(14-((55,8S,10aR)-8-(((S)-5-amino-1-(benzhydrylamino)-1,5-dioxopentan-2-yl) carbamoyl)-5-((tert-butoxycarbonyl)amino)-6-oxodecahydropyrrolo[1,2-a][1,5] diazocine-3-carboxamido)tetradecyl)-1-(isoquinolin-4-yl) piperidine-3-carboxamido)-2-oxopyridin-1 (2H)-yl)acetate (170 mg, 0.133 mmol) in THF (2 mL) and H₂O (2 mL) was added LiOH (13 mg, 0.532 mmol) at rt. The resulting mixture was stirred for 1 hr at rt. On completion, the mixture was acidified to pH 4 with aq. HCl. The residue was then purified by reversed-phase flash chromatography (column: C18 silica gel; mobile phase, MeCN in Water (10 mmol/L NH₄HCO3), 30% to 60% gradient in 30 min; detector, UV 254 nm) to afford the title compound (150 mg, 89% yield) as a light yellow solid. 1H NMR (400 MHZ, CDC13)δ 8.87 (s, 1H), 8.07 (s, 1H), 7.98-7.84 (m, 2H), 7.80-7.69 (m, 1H), 7.63-7.50 (m, 2H), 7.44-7.16 (m, 12l H), 6.78-6.33 (m, 3H), 6.23 (d, J=8.0 Hz, 1H), 5.94-5.68 (m, 2H), 4.65-4.52 (m, 1H), 4.41 (t, J=8.8 Hz, 1H), 4.34-4.25 (m, 1H), 4.19-3.98 (m, 2H), 3.79-3.75 (m, 1H), 3.45-3.31 (m, 4H), 3.29-3.18 (m, 1H), 3.11-2.95 (m, 5H), 2.52-2.39 (m, 1H), 2.35-2.14 (m, 5H), 2.11-1.85 (m, 4H), 1.86-1.71 (m, 3H), 1.65-1.54 (m, 3H), 1.46 (s, 9H), 1.40-1.15 (m, 26l H). LC/MS (ESI, m/z): [(M+1)]+=1264.7.

Step 3-2-(5-((S)—N-(14-((5S,8S,10aR)-5-amino-8-(((S)-5-amino-1-(benzhydrylamino)-1,5-dioxopentan-2-yl) carbamoyl)-6-oxodecahydropyrrolo[1,2-a][1,5] diazocine-3-carboxamido)tetradecyl)-1-(isoquinolin-4-yl) piperidine-3-carboxamido)-2-oxopyridin-1 (2H)-yl) acetic acid. To a stirred solution of 2-(5-((S)—N-(14-((5S,8S,10aR)-8-(((S)-5-amino-1-(benzhydrylamino)-1,5-dioxopentan-2-yl) carbamoyl)-5-((tert-butoxycarbonyl)amino)-6-oxodecahydropyrrolo[1,2-a][1,5] diazocine-3-carboxamido)tetradecyl)-1-(isoquinolin-4-yl) piperidine-3-carboxamido)-2-oxopyridin-1 (2H)-yl) acetic acid (130 mg, 0.102 mmol) in DCM (3 mL) was added HCl (gas) in 1,4-dioxane (1 mL) at rt. The resulting mixture was stirred for 1 hr at rt. On completion, the mixture was concentrated under vacuum to afford the title compoound (100 mg, 84% yield) as a light yellow solid. LC/MS (ESI, m/z): [(M+1)]+=1164.7.

### Methyl 2-(5-amino-2-oxopyridin-1-yl)acetate (Intermediate AN)

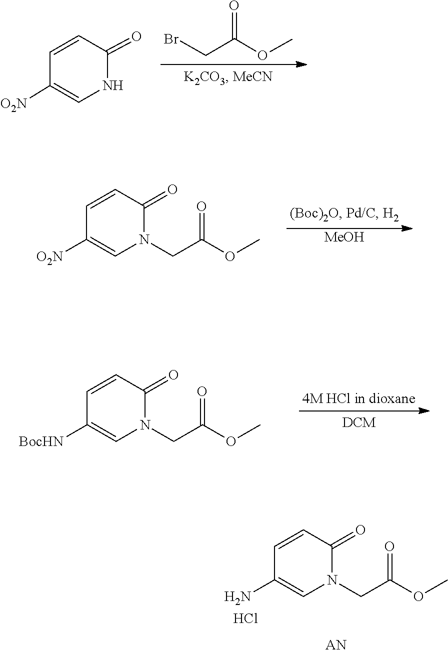

Step 1-Methyl 2-(5-nitro-2-oxopyridin-1-yl)acetate. To a stirred solution of 5-nitro-1H-pyridin-2-one (50 g, 350 mmol) and methyl 2-bromoacetate (60.06 g, 392.6 mmol) in MeCN (1.5 L) was added K₂CO₃ (73.99 g, 535.3 mmol) at rt under nitrogen atmosphere. The resulting mixture was stirred for 2 h at 70° C. under nitrogen atmosphere. On completion, the mixture was cooled to rt and filtered, and the filter cake was washed with DCM (3×20 mL). The filtrate was concentrated under reduced pressure. The residue was purified by silica gel column chromatography, eluted with Petroleum ether/EtOAc (20:1), to afford the title compound (75 g, 96% yield) as a yellow solid. 1H NMR (400 MHZ, DMSO-d₆) δ 9.26 (d, J=3.1 Hz, 1H), 8.20 (d, J=10.0 Hz, 1H), 6.56 (d, J=10.0 Hz, 1H), 4.90 (s, 2H), 3.71 (s, 3H). LC/MS (ESI, m/z): [(M+H)]+=213.1.

Step 2-Methyl 2-{5-[(tert-butoxycarbonyl)amino]-2-oxopyridin-1-yl} acetate. To a solution of methyl 2-(5-nitro-2-oxopyridin-1-yl)acetate (60 g, 280 mmol) and (Boc)₂₀ (67.89 g, 311.1 mmol) in MeOH (400 mL) was added Pd/C (1 g) under nitrogen atmosphere. The reaction system was degassed under vacuum and purged with H₂ several times, then the mixture was hydrogenated under H₂ balloon (˜1 atm) at 25° C. for 8 h. After completion of the reaction, the Pd/C was filtered off through celite. The filter cake was washed with MeOH (3×10 mL). The corresponding filtrate was concentrated under reduced pressure to afford the title compound (76 g, 95% yield) as a yellow-green solid. 1H NMR (400 MHZ, DMSO-d₆) δ 9.07 (s, 1H), 7.90 (s, 1H), 7.40 (dd, J=9.7, 2.8 Hz, 1H), 6.40 (d, J=9.7 Hz, 1H), 4.70 (s, 2H), 3.67 (s, 3H), 1.45 (s, 9H). LC/MS (ESI, m/z): [(M+H)]+=283.1.

Methyl 2-(5-amino-2-oxopyridin-1-yl)acetate hydrochloride. To a stirred solution of methyl 2-{5-[(tert-butoxycarbonyl)amino]-2-oxopyridin-1-yl} acetate (79 g, 280 mmol) in DCM (200 mL) was 4 M HCl (gas) in 1,4-dioxane (200 mL) dropwise at rt under nitrogen atmosphere. The resulting mixture was stirred for 2 h at rt under nitrogen atmosphere. On completion, the mixture was concentrated under reduced pressure. The crude was purified by triturated with Et2O to afford the title compound (50 g, 82% yield) as a green solid. LC/MS (ESI, m/z): [(M+H)]+=183.1.

### 3-(5-Bromopyridin-3-yl)benzoic acid (Intermediate AO)

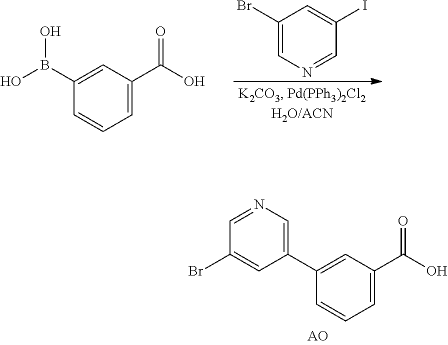 [SMILES: O=C(O)c1cccc(B(O)O)c1 ; Brc1cncc(I)c1 ; O=C(O)c1cccc(-c2cncc(Br)c2)c1]

To a solution of 3-bromo-5-iodopyridine (30.00 g, 105.5 mmol) and 3-(dihydroxyboranyl)benzoic acid (17.54 g) in H₂O (250.00 mL) and MeCN (500.00 mL) were added K₂CO₃ (29.21 g, 211.4 mmol) and Pd (PPh3)₂Cl2 (7.42 g, 10.6 mmol). The resulting solution was stirred for 3 h at 70° C. under a nitrogen atmosphere. On completion, the mixture was cooled to rt and filtered, and the filter cake was washed with MeCN (3×25 mL). The filtrate was concentrated under reduced pressure. The mixture was then acidified to pH 4 with HCl (aq.). The precipitated solids were collected by filtration and washed with H₂O (3×25 mL). The resulting solid was dried under reduced pressure to afford the title compound (30.2 g) as a white solid. 1H NMR (400 MHZ, DMSO-d₆) δ 13.20 (s, 1H), 8.93 (d, J=2.0 Hz, 1H), 8.75 (d, J=2.1 Hz, 1H), 8.47-8.40 (m, 1H), 8.25 (t, J=1.8 Hz, 1H), 8.09-7.94 (m, 2H), 7.66 (t, J=7.7 Hz, 1H); LC/MS (ESI, m/z): [(M+H) |+=278.0, 280.0.

### Methyl 2-{5-[3-(5-bromopyridin-3-yl)benzamido]-2-oxopyridin-1-yl} acetate (Intermediate AP)

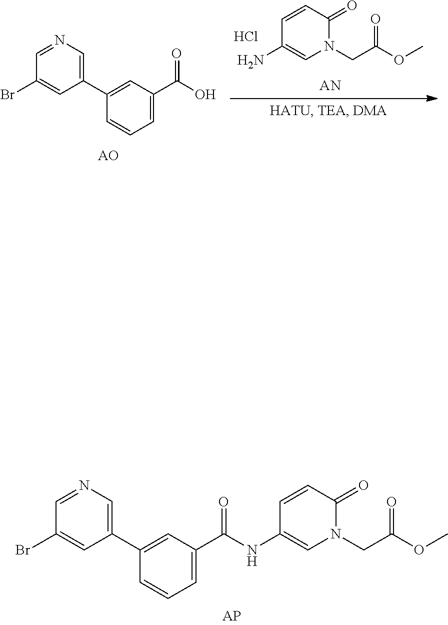 [SMILES: O=C(O)c1cccc(-c2cncc(Br)c2)c1 ; Cl ; COC(=O)Cn1cc(N)ccc1=O ; COC(=O)Cn1cc(NC(=O)c2cccc(-c3cncc(Br)c3)c2)ccc1=O]

To a stirred mixture of 3-(5-bromopyridin-3-yl)benzoic acid (20 g, 72 mmol, Intermediate AO) and methyl 2-(5-amino-2-oxopyridin-1-yl)acetate hydrochloride (17.30 g, 79.11 mmol, Intermediate AN) in DMA (200 mL) were added HATU (35.55 g, 93.49 mmol) and TEA (49.98 mL, 359.6 mmol) at 25° C. under nitrogen atmosphere. The resulting mixture was stirred for 2 h at rt under nitrogen atmosphere. On completion, the reaction was quenched by the addition of sat. NaHCO₃(aq.)(300 mL) at rt. The resulting mixture was extracted with CH₂Cl₂ (3×500 mL). The combined organic layers were washed with brine (3×200 mL), and dried over anhydrous Na₂SO₄. After filtration, the filtrate was concentrated under reduced pressure. The residue was purified by silica gel column chromatography, eluted with DCM: MeOH (20:1 to 10:1) to afford the title compound (17.6 g, 55% yield) as a green solid. 1H NMR (400 MHZ, DMSO-d₆) δ 10.18 (s, 1H), 9.01 (d, J=2.0 Hz, 1H), 8.76 (d, J=2.2 Hz, 1H), 8.49 (t, J=2.1 Hz, 1H), 8.31 (d, J=1.9 Hz, 1H), 8.26 (d, J=2.9 Hz, 1H), 8.06-7.96 (m, 2H), 7.74-7.64 (m, 2H), 6.51 (d, J=9.7 Hz, 1H), 4.77 (s, 2H), 3.70 (s, 3H). LC/MS (ESI, m/z): [(M+H)]+=442.0, 444.0.

### Methyl 2-(5-{3-[5-(9-aminononyl)pyridin-3-yl]benzamido}-2-oxopyridin-1-yl)acetate (Intermediate AQ)

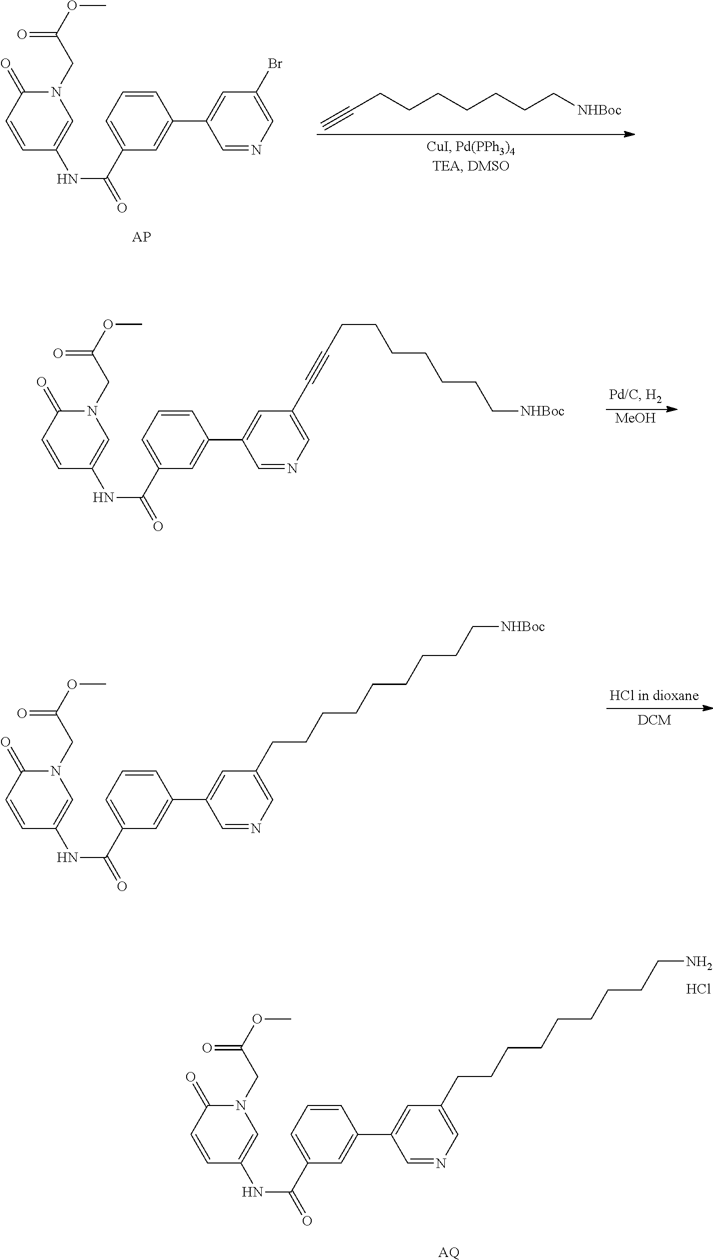

Step 1-Methyl 2-{5-[3-(5-{9-[(tert-butoxycarbonyl)amino] non-1-yn-1-yl}pyridin-3-yl)benzamido]-2-oxopyridin-1-ylacetate. To a stirred solution of methyl 2-{5-[3-(5-bromopyridin-3-yl)benzamido]-2-oxopyridin-1-yl} acetate (1.00 g, 2.26 mmol, Intermediate AP) and tert-butyl N-(non-8-yn-1-yl) carbamate (595.33 mg, 2.487 mmol, CAS #1903797-81-8) in DMSO (10 mL) were added Pd (PPh3)₄ (261.28 mg, 0.226 mmol), TEA (3 mL) and Cul (43.06 mg, 0.226 mmol) in turns at 80° C. under nitrogen atmosphere. The resulting mixture was stirred for 2 h at 80° C. under nitrogen atmosphere. On completion, the reaction mixture was concentrated under reduced pressure. The residue was purified by reverse phase flash chromatography (Column: WelFlash™ C18-I, 20-40 um, 330 g; Eluent A: Water (plus 10 mmol/L FA); Eluent B: ACN; Gradient: 25%-55% B in 25 min; Flow rate: 100 mL/min; Detector: 220/254 nm; desired fractions were collected at 50% B) and concentrated under reduced pressure to afford the title compound (793 mg, 58.38%) as a light brown solid. 1H NMR (400 MHZ, Chloroform-d) & 8.94 (s, 1H), 8.61 (s, 1H), 8.34-8.31 (m, 1H), 8.13-8.11 (m, 1H), 7.98-7.91 (m, 2H), 7.75-7.68 (m, 1H), 7.60-7.56 (m, IH), 7.49-7.43 (m, 1H), 6.53-6.51 (m, 1H), 4.69-4.65 (m, 2H), 4.59-4.55 (m, 1H), 3.74 (s, 3H), 3.13-3.08 (m, 2H), 2.47-2.43 (m, 2H), 1.69-1.59 (m, 2H), 1.51-1.47 (m, 5H), 1.43 (s, 9H), 1.42-1.35 (m, 4H). LC/MS (ESI, m/z): [(M+H)]+=601.4.

Step 2-Methyl 2-{5-[3-(5-{9-[(tert-butoxycarbonyl)amino] nonyl} pyridin-3-yl)benzamido]-2-oxopyridin-1-yl} acetate. To a solution of methyl 2-{5-[3-(5-{9-[(tert-butoxycarbonyl)amino] non-1-yn-1-yl}pyridin-3-yl)benzamido]-2-oxopyridin-1-yl} acetate (800 mg, 1.33 mmol) in MeOH (10 mL) was added Pd/C (200 mg) under nitrogen atmosphere. The reaction system was degassed under vacuum and purged with H₂ several times. Then the mixture was hydrogenated under H₂ balloon (˜1 atm) at rt for 3 hrs. After completion of the reaction, Pd/C was filtered off through celite. The filter cake was washed with MeOH (3×10 mL). The corresponding filtrate was concentrated under reduced pressure to afford the title compound (500 mg, 62% yield) as a light brown solid. 1H NMR (400 MHZ, Chloroform-d)δ 9.00-8.97 (m, 1H), 8.67 (s, 1H), 8.43 (s, 1H), 8.35-8.31 (m, 1H), 8.12 (s, 1H), 7.95-7.91 (m, 1H), 7.75-7.71 (m, 2H), 7.65-7.52 (m, 2H), 7.48-7.45 (m, 1H), 6.56-6.52 (m, 1H), 3.75 (s, 3H), 3.10-3.05 (m, 2H), 2.69-2.63 (m, 2H), 1.67-1.64 (m, 2H), 1.46-1.40 (m, 13l H), 1.32-1.25 (m, 10l H). LC/MS (ESI, m/z): [(M+H)]+=605.4.

Step 3-2-(5-{3-[5-(9-aminononyl)pyridin-3-yl]benzamido}-2-oxopyridin-1-yl)acetate hydrochloride. To a stirred solution of methyl 2-{5-[3-(5-{9-[(tert-butoxycarbonyl)amino] nonyl} pyridin-3-yl)benzamido]-2-oxopyridin-1-yl} acetate (400 mg, 0.661 mmol) in DCM (6 mL) was added HCl (gas) in 1,4-dioxane (2 mL) in portions at rt under air atmosphere. The resulting mixture was stirred for 1 h at rt under air atmosphere. On completion, the reaction mixture was concentrated under reduced pressure. The residue was purified by trituration with Et2O and filtered and dried to afford the title compound (300 mg, 90% yield) as a light brown solid. 1H NMR (400 MHZ, DMSO-d₆) § 10.74 (s, 1H), 9.36-9.32 (m, 1H), 9.01 (s, 1H), 8.83 (s, 1H), 8.65 (s, 1H), 8.38-8.34 (m, 1H), 8.17-8.05 (m, 3H), 7.95-7.91 (m, 1H), 7.77-7.66 (m, 1H), 6.52-6.48 (m, 1H), 4.79-4.75 (m, 2H), 3.69 (s, 3H), 2.89-2.86 (m, 2H), 2.75-2.71 (m, 2H), 1.74-1.70 (m, 2H), 1.56-1.49 (m, 2H), 1.33-1.28 (m, 11l H); LC/MS (ESI, m/z): [(M+H)]+=505.3.

### {5-[3-(5-{9-[(5S,8S,10aR)-5-Amino-8-{[(1S)-3-carbamoyl-1-{[(4-isopropylphenyl)methyl|carbamoyl} propyl] carbamoyl}-6-oxo-octahydropyrrolo[1,2-a][1,5] diazocine-3-carbonylamino] nonyl} pyridin-3-yl)benzamido]-2-oxopyridin-1-yl} acetic acid (Intermediate AS)

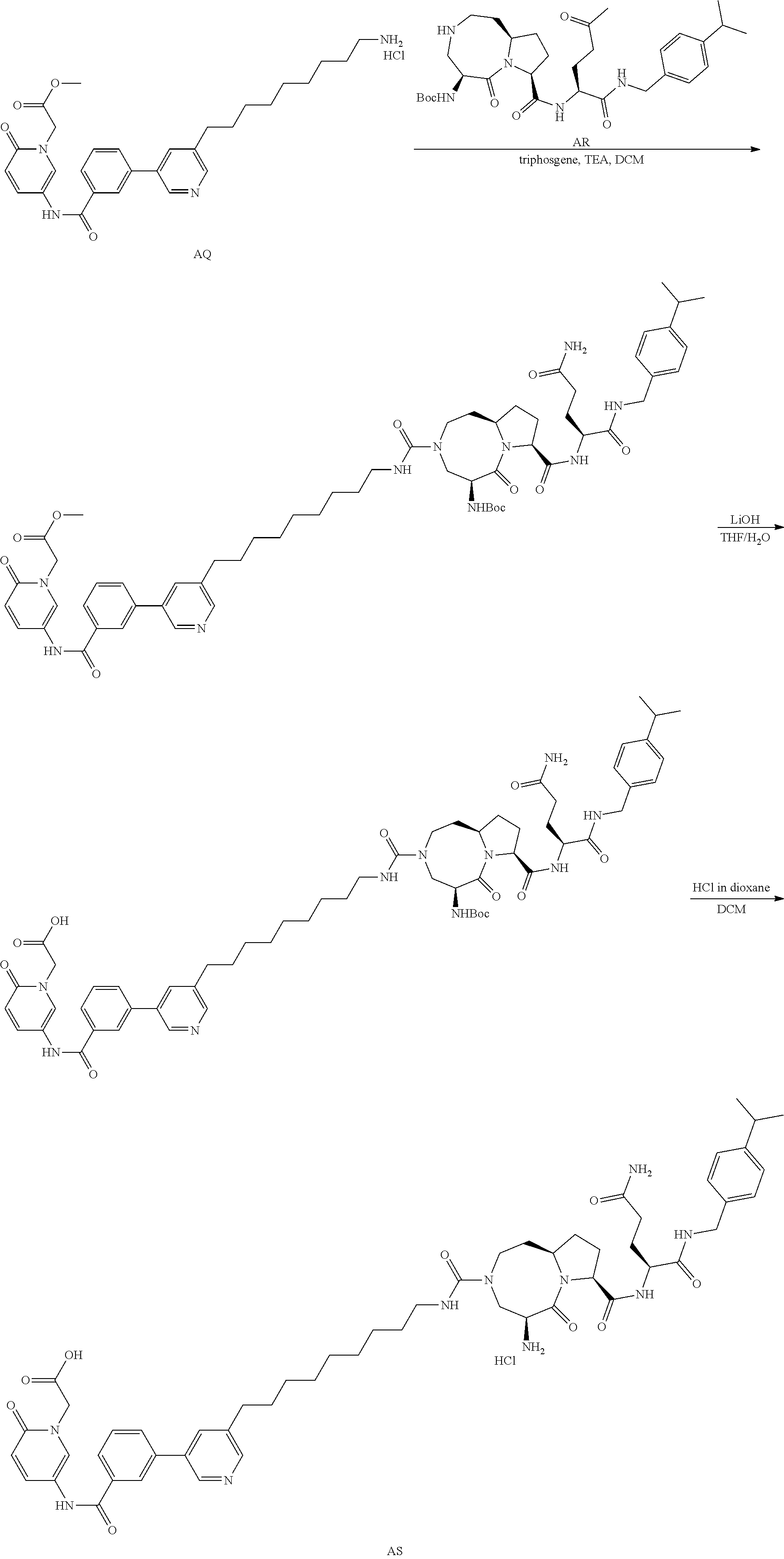

Step 1-Methyl 2-{5-[3-(5-{9-[(5S,8S,10aR)-5-[(tert-butoxycarbonyl)amino]-8-{[(1S)-3-carbamoyl-1-{[(4-isopropylphenyl)methyl] carbamoyl} propyl] carbamoyl}-6-oxo-octahydropyrrolo[1,2-a] [1,5] diazocine-3-carbonylamino] nonyl} pyridin-3-yl)benzamido]-2-oxopyridin-1-yl} acetate. To a stirred solution of triphosgene (63.51 mg, 0.214 mmol) and tert-butyl N-[(5S,8S,10aR)-8-{[(1S)-3-carbamoyl-1-{[(4-isopropylphenyl)methyl] carbamoyl} propyl] carbamoyl}-6-oxo-octahydro-1H-pyrrolo[1,2-a][1,5] diazocin-5-yl]carbamate (313.93 mg, 0.535 mmol, Intermediate AR, synthesis described in WO 2021/188696) and TEA (189.49 mg, 1.873 mmol) in DCM (14 mL) was added methyl 2-(5-{3-[5-(9-aminononyl)pyridin-3-yl]benzamido}-2-oxopyridin-1-yl)acetate hydrochloride (270 mg, 0.500 mmol, Intermediate AQ) in portions at rt under air atmosphere. The resulting mixture was stirred for 2 h at rt under air atmosphere. On completion, the reaction mixture was concentrated under vacuum. The residue was purified by reverse phase flash chromatography (Column: WelFlash™ C18-I, 20-40 um, 120 g; Eluent A: Water (plus 10 mmol/L FA); Eluent B: ACN; Gradient: 25%-55% B in 25 min; Flow rate: 60 mL/min; Detector: 220/254 nm; desired fractions were collected at 53% B) and concentrated under reduced pressure to afford the title compound (212.2 mg, 38% yield) as a light brown solid. 1H NMR (400 MHZ, Chloroform-d) ô 9.41 (s, 1H), 8.77 (s, 1H), 8.40 (s, 1H), 8.39-8.35 (m, 1H), 8.24 (s, 1H), 7.99-7.97 (m, 1H), 7.84-7.72 (m, 2H), 7.66-7.62 (m, 1H), 7.58-7.55 (m, 1H), 7.43-7.40 (m, 1H), 7.30-7.31 (m, 1H), 7.18-7.15 (m, 6H), 6.58-6.54 (m, 1H), 5.90-5.88 (m, 1H), 4.81-4.77 (m, 1H), 4.69 (s, 1H), 4.52-4.48 (m, 1H), 4.39-4.35 (m, 4H), 4.30-4.21 (m, 2H), 4.07-4.04 (m, 2H), 3.76 (s, 3H), 3.39-3.35 (m, 2H), 3.19-3.13 (m, 1H), 2.91-2.86 (m, 5H), 2.71-2.67 (m, 1H), 2.44-2.39 (m, 1H), 2.25-2.19 (m, 8H), 1.76-1.64 (m, 2H), 1.45 (s, 9H), 1.41-1.38 (m, 2H), 1.31-1.27 (m, 6H), 1.24-1.22 (m, 10l H); LC/MS (ESI, m/z): [(M+H)]+=1117.7.

Step 2-{5-[3-(5-{9-[(5S,8S,10aR)-5-[(tert-butoxycarbonyl)amino]-8-{[(1S)-3-carbamoyl-1-{[(4-isopropylphenyl)methyl] carbamoyl} propyl] carbamoyl}-6-oxo-octahydropyrrolo[1,2-a] [1,5] diazocine-3-carbonylamino] nonyl} pyridin-3-yl)benzamido]-2-oxopyridin-1-yl} acetic acid. To a stirred solution of methyl 2-{5-[3-(5-{9-[(55,85,10aR)-5-[(tert-butoxycarbonyl)amino]-8-{[(1S)-3-carbamoyl-1-{[(4-isopropylphenyl)methyl] carbamoyl} propyl] carbamoyl}-6-oxo-octahydropyrrolo[1,2-a] [1,5] diazocine-3-carbonylamino] nonyl} pyridin-3-yl)benzamido]-2-oxopyridin-1-yl} acetate (350 mg, 0.313 mmol) in H₂O (3 mL) were added THF (3 mL) and LiOH (75.01 mg, 3.132 mmol) in turns at rt under air atmosphere. The resulting mixture was stirred for 1 hr at rt under air atmosphere. On completion, the reaction mixture was acidified with FA. The resulting mixture was concentrated under vacuum. The residue was purified by reverse phase flash chromatography (Column: WelFlash™ C18-I, 20-40 um, 120 g; Eluent A: Water (plus 10 mmol/L FA); Eluent B: ACN; Gradient: 25%-55% B in 25 min; Flow rate: 60 mL/min; Detector: 220/254 nm; desired fractions were collected at 47% B) and concentrated under reduced pressure to afford the title compound (212 mg, 61) as a light brown solid. 1H NMR (400 MHZ, DMSO-d₆) ô 13.00 (s, 1H), 10.17 (s, 1H), 8.83-8.79 (m, 1H), 8.49-8.46 (m, 1H), 8.34-8.24 (m, 2H), 8.23-8.13 (m, 2H), 8.03-7.92 (m, 3H), 7.72-7.62 (m, 2H), 7.21-7.18 (m, 1H), 7.14-7.17 (m, 3H), 6.76-6.64 (m, 1H), 6.49-6.44 (m, 3H), 4.68-4.64 (m, 2H), 4.46-4.35 (m, 2H), 4.29-4.16 (m, 4H), 3.78-3.74 (m, 2H), 3.15-3.12 (m, 1H), 3.04-3.01 (m, 1H), 2.91-2.74 (m, 3H), 2.73-2.65 (m, 2H), 2.21-2.08 (m, 4H), 1.95-1.91 (m, 1H), 1.83-1.76 (m, 1H), 1.68-1.59 (m, 3H), 1.49-1.43 (m, 4H), 1.38 (s, 9H), 1.32-1.28 (m, 12l H), 1.20-1.15 (m, 6H); LC/MS (ESI, m/z): [(M+H)]+=1103.7.

Step 3 {5-[3-(5-{9-[(5S,8S,10aR)-5-amino-8-{[(1S)-3-carbamoyl-1-{[(4-isopropylphenyl)methyl] carbamoyl} propyl] carbamoyl}-6-oxo-octahydropyrrolo[1,2-a][1,5] diazocine-3-carbonylamino] nonyl} pyridin-3-yl)benzamido]-2-oxopyridin-1-yl} acetic acid hydrochloride. To a stirred solution of {5-[3-(5-{9-[(5S,8S,10aR)-5-[(tert-butoxycarbonyl)amino]-8-{[(1S)-3-carbamoyl-1-{[(4-isopropylphenyl)methyl|carbamoyl} propyl] carbamoyl}-6-oxo-octahydropyrrolo[1,2-a][1,5] diazocine-3-carbonylamino] nonyl} pyridin-3-yl)benzamido]-2-oxopyridin-1-yl} acetic acid (180 mg, 0.163 mmol) in DCM (3 mL) was added HCl (gas) in 1,4-dioxane (2 mL) dropwise at rt. The resulting mixture was then stirred for 30 min at rt. On completion, the mixture was concentrated under reduced pressure. The residue was purified by trituration with Et20, filtered and dried to afford the title compound (143 mg, 84% yield) as a light brown solid. 1H NMR (400 MHz, DMSO-d₆) § 13.00 (s, 1H), 10.17 (s, 1H), 8.83-8.79 (m, 1H), 8.49-8.45 (m, 1H), 8.34-8.24 (m, 2H), 8.24-8.15 (m, 2H), 8.03-7.92 (m, 3H), 7.72-7.62 (m, 2H), 7.21 (s, 1H), 7.18-7.14 (m, 3H), 6.73 (s, 1H), 6.48-6.44 (m, 3H), 4.67 (s, 2H), 4.46-4.37 (m, 2H), 4.29-4.16 (m, 4H), 3.80-3.72 (m, 3H), 3.19-3.08 (m, 2H), 3.07-2.94 (m, 2H), 2.93-2.74 (m, 2H), 2.74-2.66 (m, 2H), 2.21-2.08 (m, 3H), 1.95-1.91 (m, 1H), 1.83-1.76 (m, 1H), 1.65-1.59 (m, 4H), 1.49-1.43 (m, 3H), 1.34-1.26 (m, 12l H), 1.20-1.16 (m, 6H); LC/MS (ESI, m/z): [(M+H)]+=1003.5.

### 2-(2,3,4,5,6-pentafluorophenoxycarbonyl)-1H-indole-5-carbonylphosphonic acic (Intermediate AT)

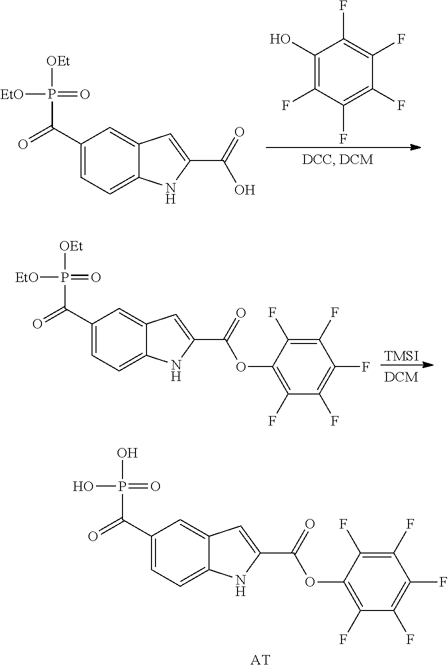 [SMILES: CCOP(=O)(OCC)C(=O)c1ccc2[nH]c(C(=O)O)cc2c1 ; "TMSI" ; Oc1c(F)c(F)c(F)c(F)c1F ; CCOP(=O)(OCC)C(=O)c1ccc2[nH]c(C(=O)Oc3c(F)c(F)c(F)c(F)c3F)cc2c1 ; O=C(Oc1c(F)c(F)c(F)c(F)c1F)c1cc2cc(C(=O)P(=O)(O)O)ccc2[nH]1 ; "AT"]

Step 1-2,3,4,5,6-Pentafluorophenyl 5-[(diethoxyphosphoryl) carbonyl]-1H-indole-2-carboxylate. To a stirred solution of 5-[(diethoxyphosphoryl) carbonyl]-1H-indole-2-carboxylic acid (100.00 g, 307.45 mmol, CAS #2502205-58-3) and pentafluorophenol (84.89 g, 461.2 mmol) in DCM (1.50 L) was added DCC (95.15 g, 461.2 mmol) at rt under air atmosphere. The resulting mixture was stirred overnight at rt under nitrogen atmosphere. On completion, the mixture was filtered and the filter cake was washed with DCM (3×100 mL). The filtrate was concentrated under reduced pressure to 300 mL of DCM. The resulting mixture was diluted with hexane (1 L). The precipitated solids were collected by filtration and washed with hexane (3×100 mL) to afford the title compound (150 g) as a light yellow solid. LC/MS (ESI, m/z): [(M+H)]+=492.1.

Step 2-2-(2,3,4,5,6-Pentafluorophenoxycarbonyl)-1H-indole-5-carbonylphosphonic acid. To a stirred solution of 2,3,4,5,6-pentafluorophenyl 5-[(diethoxyphosphoryl) carbonyl]-1H-indole-2-carboxylate (65.00 g, 132.3 mmol) in anhydrous DCM (1300 mL) was added TMSI (79.42 g, 396.9 mmol) dropwise at rt under Argon atmosphere. The resulting mixture was stirred for 30 min at rt under Argon atmosphere. On completion, the mixture was concentrated under reduced pressure. The residue was dissolved in dry MeCN (500 mL) then sat. aq. Na₂S203 (50 mL) was added dropwise into the solution until the dark brown solution converted to light yellow. A precipitate was formed and the suspension was filtered. The filter cake was washed with ACN/water (10/1, 50 mL, three times) and collected. The collected solid was triturated with Et2O (500 ml), filtered and dried in vacuo to afford the title compound (45 g, 78% yield) as an off-white solid. LC/MS (ESI, m/z): [(M+H)]+=436.0; 1H NMR (300 MHz, DMSO-d₆) δ 12.84 (s, 1H), 8.98 (d, J=1.6 Hz, 1H), 8.08 (dd, J=8.8, 1.6 Hz, 1H), 7.76 (d, J=1.9 Hz, 1H), 7.59 (d, J=8.8 Hz, 1H).

### Benzyl(S)-3-((1-(2-methoxy-2-oxoethyl)-6-oxo-1,6-dihydropyridin-3-yl) carbamoyl) piperidine-1-carboxylate (Intermediate AU)

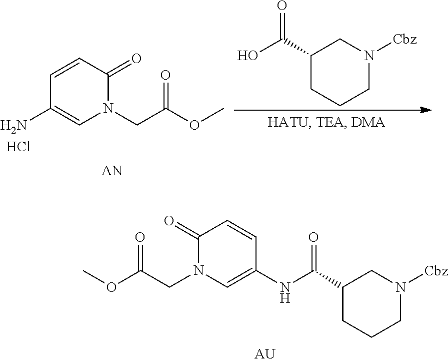

To a stirred solution of methyl 2-(5-amino-2-oxopyridin-1-yl) acctatc hydrochloridc (5 g, 22.869 mmol, Intermediate AN) and(S)-1-((benzyloxy) carbonyl) piperidine-3-carboxylic acid (6.62 g, 25.2 mmol, CAS #88466-74-7) in DMA (100 mL) were added DIEA (11.82 g, 91.48 mmol) and HATU (10.43 g, 27.44 mmol) at 0° C. under nitrogen atmosphere. The resulting mixture was stirred for 3 hr at rt under nitrogen atmosphere. On completion, the reaction mixture was diluted with water (800 mL) and extracted with EtOAc (3×300 mL). The combined organic layers were washed with brine (2×150 mL), and dried over anhydrous Na₂SO₄. After filtration, the filtrate was concentrated under reduced pressure. The residue was purified by reverse flash chromatography (column, WelFlash™ C18-I,20-40 um,330g; Eluent A: Water (plus 10 mmol/L NH₄CO₃);Eluent B: ACN, 20% to 40% gradient in 25 min; Flow rate: 80 mL/min; detector: UV 220/254 nm, desired fractions were collected at 38% B) and concentrated under reduced pressure to afford the title compound (6 g, 61% yield) as a light yellow solid. LC/MS (ESI, m/z): [(M+H)] +=428.2.

### Tert-butyl (3S)-3-{[1-(2-methoxy-2-oxoethyl)-6-oxopyridin-3-yl]carbamoyl} piperidine-1-carboxylate (Intermediate AV)

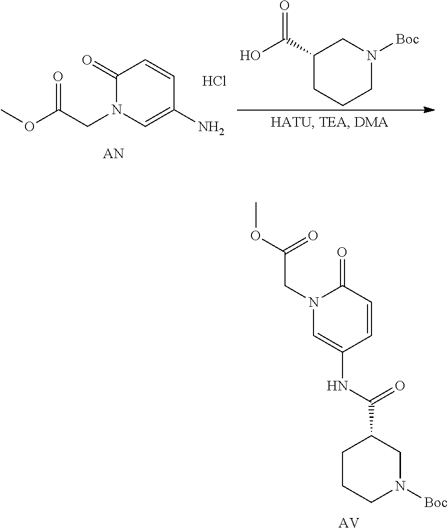

To a stirred solution of methyl 2-(5-amino-2-oxopyridin-1-yl)acetate hydrochloride (10 g, 50 mmol, Intermediate AN), TEA (19.07 mL, 137.211 mmol) and (3S)-1-(tert-butoxycarbonyl) piperidine-3-carboxylic acid (10.49 g, 45.75 mmol, CAS #88495-54-9) in DMA (100 mL) was added HATU (20.87 g, 54.884 mmol) at 0° C. The resulting mixture was then stirred for 3 hr at rt. On completion, the mixture was diluted with water (600 mL) and extracted with EtOAc (3×400 mL). The combined organic layers were washed with brine (3×400 mL), and dried over anhydrous Na₂SO₄. After filtration, the filtrate was concentrated under reduced pressure. The residue was purified by reversed-phase flash chromatography (column, C18 silica gel; mobile phase, MeCN in Water (10 mmol/L NH₄HCO3), 20% to 50% gradient in 30 min; detector, UV 254 nm) to afford the title compound (9 g, 50% yield) as a light-dark solid. LC/MS (ESI, m/z): [(M+1)]+=394.2; 1H NMR (400 MHZ, Chloroform-d)δ 8.76 (s, 1H), 8.32 (d, J=2.7 Hz, 1H), 7.32 (dd, J=9.7, 2.8 Hz, 1H), 6.58 (d, J=9.7 Hz, 1H), 4.65 (d, J=1.7 Hz, 2H), 3.78 (s, 3H), 3.75-3.70 (m, 1H), 3.65-3.49 (m, 2H), 3.39-3.23 (m, 1H), 2.56-2.48 (m, 1H), 2.18-2.05 (m, 1H), 1.93-1.86 (m, 1H), 1.67-1.57 (m, 1H), 1.54-1.44 (m, 10l H).

### Tert-butyl 2-(2-oxo-5-(3-(4,4,5,5-tetramethyl-1,3,2-dioxaborolan-2-yl)benzamido)pyridin-1 (2H)-yl)acetate (Intermediate AW)

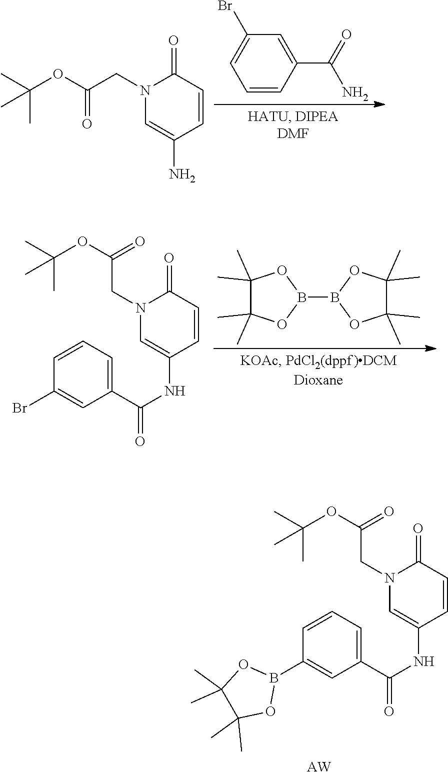

Step 1-Tert-butyl 2-(5-(3-bromobenzamido)-2-oxopyridin-1 (2H)-yl)acetate. To a stirred solution of 3-bromobenzoic acid (1.00 g, 5.00 mmol) in DMF (20 mL) was added DIPEA (2.7 mL, 15.00 mmol) followed by HATU (2.85 g, 7.50 mmol) at 0° C. and the reaction mixture was stirred at rt for 15 min. Next, tert-butyl 2-(5-amino-2-oxopyridin-1 (2H)-yl)acetate (1.67 g, 7.50 mmol, synthesized as described for Intermediate AN with tert-butyl 2-bromoacetate and 5-nitropyridin-2 (1H)-one used in Step 1) was added and the reaction mixture was stirred at rt for 12 h. After completion of reaction, the reaction mixture was diluted with water and the aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (80% EtOAc/hexane) to afford the title compound (1.30 g, 65% yield) as a dark brown solid. 1H NMR (400 MHZ, DMSO-d₆) δ 10.18 (s, 1H), 8.20 (brs, 1H), 8.12 (brs, 1H), 7.93 (d, J=7.34 Hz, 1H), 7.80 (d, J=8.31 Hz, 1H), 7.65 (d, J=9.29 Hz, 1H), 7.50 (t, J=7.34 Hz, 1H), 6.46 (d, J-9.78 Hz, 1H), 4.62 (s, 2H), 1.43 (s, 9H); LC-MS: m/z 350.9 [M-56+H]+.

Step 2 Tert-butyl 2-(2-oxo-5-(3-(4,4,5,5-tetramethyl-1,3,2-dioxaborolan-2-yl)benzamido)pyridin-1 (2H)-yl)acetate. To a stirred solution of tert-butyl 2-(5-(3-bromobenzamido)-2-oxopyridin-1 (2H)-yl)acetate (1.00 g, 2.46 mmol) and bispinacolato diborane (0.75 g, 2.90 mmol) in 1,4 dioxane (20 mL) was added KOAc (0.48 g, 4.90 mmol) and the reaction mixture was purged with argon for 20 min. To the resulting reaction mixture was added PdCl2 (dppf).DCM (0.20 g, 0.24 mmol) and the reaction mixture was again purged with argon for 20 min. The reaction mixture was then stirred at 90° C. for 12 h. After completion of reaction, the reaction mixture was filtered through celite and washed with ethyl acetate. The filtrate was dried over anhydrous Na₂SO₄ and concentrated under reduced pressure. The crude compound was purified by silica gel column chromatography (80% EtOAc/hexane) to afford the title compound (1.0 g, 89% yield) as a dark brown solid. 1H NMR (400 MHZ, DMSO-d₆) δ 10.18 (s, 1H), 8.23 (s, 1H), 8.18 (brs, 1H), 8.01-8.06 (m, 1H), 7.86 (d, J=6.85 Hz, 1H), 7.68 (d, J-9.78 Hz, 1H), 7.54 (t, J=7.58 Hz, 1H), 6.45 (d, J=9.29 Hz, 1H), 4.62 (s, 2H), 1.43 (s, 9H), 1.28-1.36 (m, 12l H); LC-MS: m/z 399.0 [M-56+H]+.

### 2-Bromothiophene (CAS #1003 Sep. 4)(Intermediate AX)

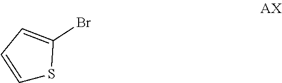

### 3-Bromo-N-methylbenzamide (Intermediate AY)

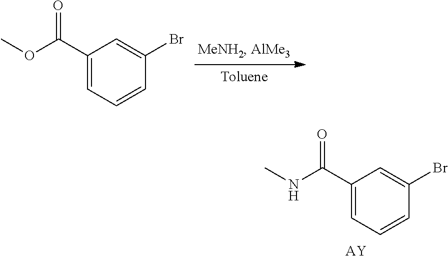

To a stirred solution of methyl 3-bromobenzoate (1.00 g, 4.30 mmol) in toluene (15 mL) was added methyl amine (2M in THF, 4.3 mL) followed by trimethyl aluminium (2M in toluene, 3.22 mL) and the reaction mixture was stirred at 90° C. for 14 h. After completion of reaction, the reaction mixture was cooled to rt and quenched with dilute HCl. The aqueous layer was then extracted with ethyl acetate. The organic layer was dried over anhydrous Na₂SO₄ and concentrated under reduced pressure. The crude compound was purified by silica gel column chromatography (50% EtOAc/hexane) to afford the title compound (0.70 g, 75% yield) as an off white solid. 1H NMR (400 MHZ, DMSO-d₆) δ 8.55 (brs, 1H), 8.00 (s, 1H), 7.83 (d, J=7.83 Hz, 1H), 7.68-7.75 (m, 1H), 7.43 (t, J=7.83 Hz, 1H), 2.78 (d, J=4.89 Hz, 3H); LC-MS: m/z 216.02 [M+2H]+.

### 1-Bromo-3-(methylsulfonyl)benzene (CAS #34896-80-5)(Intermediate AZ)

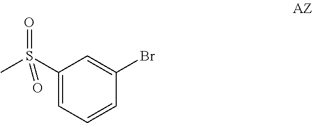

### 4-bromo-N-methylbenzamide (Intermediate BA)

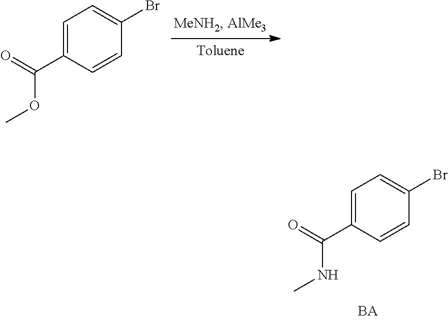

To a stirred solution of methyl 4-bromobenzoate (1.00 g, 4.30 mmol) in toluene (15 mL) was added methyl amine (2M in THF, 4.3 mL) followed by trimethyl aluminium (2M in toluene, 3.22 mL) and the reaction mixture was stirred at 90° C. for 14 h. After completion of reaction, the reaction mixture was cooled to rt and quenched with dilute HCl. The aqueous layer was extracted with ethyl acetate. The organic layer was dried over anhydrous Na₂SO₄ and concentrated under reduced pressure. The crude compound was purified by silica gel column chromatography (50% EtOAc/hexane) to afford the title compound (0.70 g, 75% yield) as an off white solid. 1H NMR (400 MHZ, DMSO-d₆) δ 8.51 (brs, 1H), 7.74-7.80 (m, 2H), 7.67 (d, J=8.80 Hz, 2H), 2.77 (d, J=4.40 Hz, 3H); LC-MS: m/z 213.8 [M]+.

### 1-Bromo-4-(methylsulfonyl)benzene (CAS #3466-32-8)(Intermediate BB)

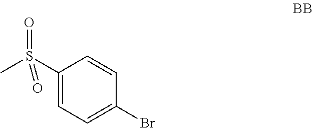

### Ethyl 2-(5-(3-iodobenzamido)-2-oxopyridin-1 (2H)-yl)acetate (Intermediate BC)

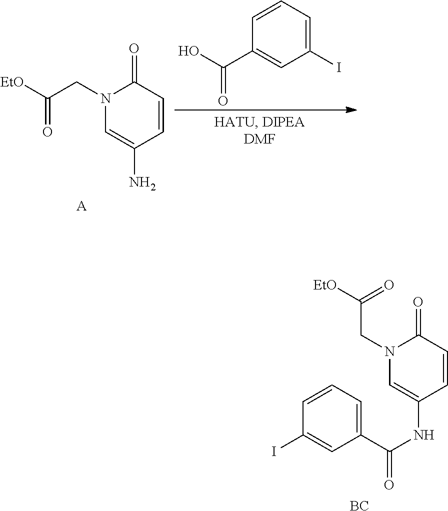

To a stirred solution of 3-iodobenzoic acid (1.26 g, 5.10 mmol) in DMF (10 mL) was added DIPEA (2.25 mL, 12.75 mmol) followed by HATU (2.32 g, 6.12 mmol) at 0° C. and the reaction mixture was stirred at rt for 15 min. Next, ethyl 2-(5-amino-2-oxopyridin-1 (2H)-yl)acetate (1.00 g, 5.10 mmol, Intermediate A) was added and the reaction mixture was stirred at rt for 16 h. After completion of reaction, the reaction mixture was diluted with water and the aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (60% EtOAc/hexane) to afford the title compound (0.80 g, 37% yield) as a dark brown solid. 1H NMR (400 MHZ, DMSO-d₆) δ 10.17 (s, 1H), 8.21-8.30 (m, 2H), 7.94 (dd, J-8.56, 9.54 Hz, 2H), 7.66 (dd, J=2.69, 9.54 Hz, 1H), 7.34 (t, J=7.83 Hz, 1H), 6.48 (d, J=9.78 Hz, 1H), 4.73 (s, 2H), 4.15 (q, J=7.34 Hz, 2H), 1.21 (t, J=7.09 Hz, 3H); LC-MS: m/z 427.2 [M+H]+.

### (3-(Trifluoromethyl)phenyl) boronic acid (CAS #1423-26-3)(Intermediate BD)

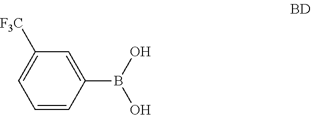 [SMILES: OB(O)c1cccc(C(F)(F)F)c1]

### (4-(Trifluoromethyl)phenyl) boronic acid (Intermediate BE)

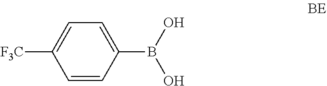 [SMILES: OB(O)c1ccc(C(F)(F)F)cc1]

### Pyrimidin-5-ylboronic acid (Intermediate BF)

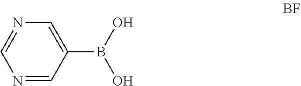

### 3-(Benzo [d] thiazol-2-yl)benzoic acid (Intermediate BG)

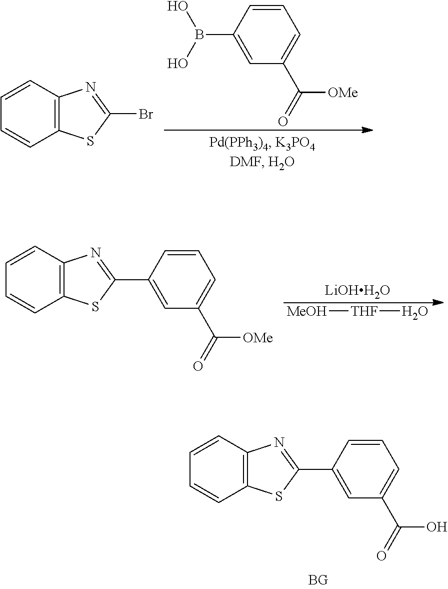

Step 1-Methyl 3-(benzo[d]thiazol-2-yl)benzoate. To a stirred solution of 2-bromobenzo[d]thiazole (0.50 g, 2.30 mmol) and (3-(methoxycarbonyl)phenyl) boronic acid (1.05 g, 5.80 mmol) in DMF (9 mL) and H₂O (1 mL), was added K₃PO4 (1.22 g, 5.80 mmol) and the reaction mixture was purged with argon for 15 min. To the resulting reaction mixture was added Pd (PPh3)₄ (0.27 g, 0.23 mmol) and the reaction mixture was again purged with argon for 15 min. The reaction mixture was stirred at 90° C. for 12 h. After completion of reaction, the reaction mixture was diluted with water and the aqueous layer was extracted with ethyl acetate. The organic layer was washed with water and brine, dried over anhydrous Na₂SO₄ and concentrated under reduced pressure. The crude compound was purified by silica gel column chromatography (10% EtOAc/hexane) to afford the title compound (0.20 g, 36% yield) as a light brown solid. LC-MS: m/z 269.85 [M+H]+.

Step 2-3-(Benzo [d] thiazol-2-yl)benzoic acid. To a stirred solution of methyl 3-(benzo[d]thiazol-2-yl)benzoate (0.20 g, 0.74 mmol) in a mixture of THF (2 mL), MeOH (2 mL) and water (1 mL) was added LiOH.H₂O (0.05 g, 1.10 mmol) and the reaction mixture was stirred at rt for 3 hr. After completion of reaction, the reaction mixture was concentrated under reduced pressure. The crude material was dissolved in water, and acidified with 1N HCl to pH 3. The aqueous layer was extracted with ethyl acetate. The organic layer was dried over anhydrous Na₂SO₄ and concentrated under reduced pressure to afford the title compound (0.16 g, 79% yield) as an off white solid. ¹H NMR (400 MHZ, DMSO-d₆) δ 13.36 (brs, 1H), 8.64 (s, 1H), 8.33 (d, J=7.83 Hz, 1H), 8.19 (d, J=7.83 Hz, 1H), 8.12 (dd, J-5.62, 7.58 Hz, 2H), 7.73 (t, J-7.58 Hz, 1H), 7.56-7.62 (m, 1H), 7.47-7.54 (m, 1H); LC-MS: m/z 255.9 [M+H]+.

### Ethyl 2-(5-(3-bromo-4-methylbenzamido)-2-oxopyridin-1 (2H)-yl)acetate (Intermediate BG)

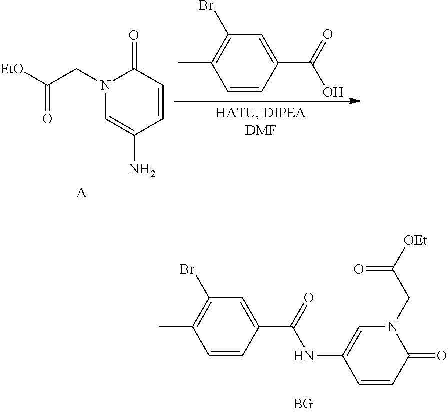

To a stirred solution of 3-bromo-4-methylbenzoic acid (0.50 g, 2.32 mmol) in DMF (5 mL) was added DIPEA (0.75 mL, 5.81 mmol), followed by HATU (1.06 g, 2.79 mmol) at 0° C., then the reaction mixture was stirred at rt for 15 min. To the resulting reaction mixture was added ethyl 2-(5-amino-2-oxo-1-pyridyl)acetate (0.46 g, 2.32 mmol, Intermediate A) and the reaction mixture was stirred at rt for 6 h. After completion of reaction, the reaction mixture was diluted with water and the aqueous layer was extracted with ethyl acetate. The organic layer was washed with saturated NaHCO₃ solution and brine, dried over anhydrous Na₂SO₄ and concentrated under reduced pressure. The crude compound was purified by silica gel column chromatography (80% EtOAc/hexane) to afford the title compound (0.30 g, 33% yield) as an off white solid. 1H NMR (400 MHZ, DMSO-d₆) δ 10.10-10.15 (m, 1H), 8.23 (d, J-2.45 Hz, 1H), 8.15 (d, J=0.98 Hz, 1H), 7.85 (dd, J=1.47, 7.83 Hz, 1H), 7.66 (dd, J-2.93, 9.78 Hz, 1H), 7.52 (d, J=8.31 Hz, 1H), 6.47 (d, J-9.78 Hz, 1H), 4.73 (s, 2H), 4.15 (q, J-6.85 Hz, 2H), 2.42 (s, 3H), 1.21 (t, J-7.34 Hz, 3H); LC-MS: m/z 395.1 [M+2H]+.

### Pyridin-3-ylboronic acid (CAS #1692-15-5)(Intermediate BH)

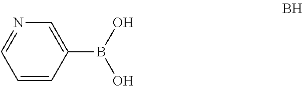

### 1-Isobutyl-1H-pyrrolo[2,3-b]pyridine-4-carboxylic acid (Intermediate BI)

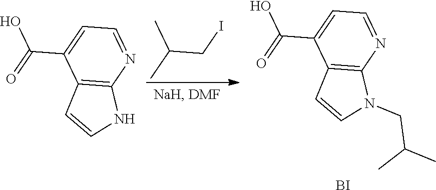

To a stirred solution of 1H-pyrrolo[2,3-b]pyridine-4-carboxylic acid (0.25 g, 1.50 mmol) in DMF (6 mL) was added NaH (0.09 g, 2.30 mmol, 60% dispersion in mineral oil) at 0° C. and the reaction mixture was stirred for 20 min. To the resulting reaction mixture was added 1-iodo-2-methylpropane (0.43 g, 2.30 mmol) and the reaction mixture was then stirred at rt for 6 h. After completion of reaction, the reaction mixture was quenched with cold water. The aqueous layer was extracted with 10% MeOH/DCM. The organic layer was dried over anhydrous Na₂SO₄ and concentrated under reduced pressure to afford the title compound (0.15 g) as brown thick liquid. LC-MS: m/z 219.10 [M+H]+.

### 1-(6-((Tert-butoxycarbonyl)amino) hexyl)-1H-indole-4-carboxylic acid (Intermediate BJ)

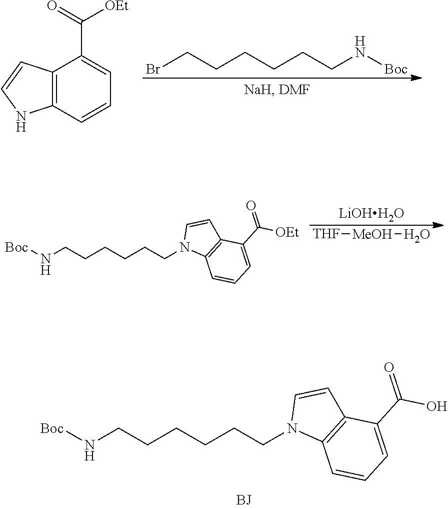

Step 1-Ethyl 1-(6-((tert-butoxycarbonyl)amino) hexyl)-1H-indole-4-carboxylate. To a stirred solution of ethyl 1H-indole-4-carboxylate (0.50 g, 2.64 mmol) in DMF (5 mL) was added NaH (0.19 g, 3.96 mmol, 60% dispersion in mineral oil) at 0° C. To the resulting reaction mixture was added tert-butyl (6-bromohexyl) carbamate (0.74 g, 2.64 mmol, CAS #142356-33-0) and the reaction mixture was stirred at rt for 2 hr. After completion of reaction, the reaction mixture was quenched with saturated NH₄Cl solution at 0° C. The aqueous layer was then extracted with ethyl acetate. The combined organic layer was dried over anhydrous Na₂SO₄ and concentrated under reduced pressure. The crude compound was purified by silica gel column chromatography (20% EtOAc/hexane) to afford the title compound (0.55 g, 54% yield) as an off white solid. 1H NMR (400 MHZ, DMSO-d₆) δ 7.71-7.82 (m, 2H), 7.54-7.59 (m, 1H), 7.23 (t, J=7.83 Hz, 1H), 6.93 (d, J=2.93 Hz, 1H), 6.75 (brs, 1H), 4.35 (q, J-6.85 Hz, 2H), 4.22 (t, J=6.85 Hz, 2H), 2.86 (q, J=6.36 Hz, 2H), 1.68-1.78 (m, 2H), 1.27-1.41 (m, 13l H), 1.21 (d, J=10.27 Hz, 3H)(2H merged in solvent peak).

Step 2-1-(6-((tert-butoxycarbonyl)amino) hexyl)-1H-indole-4-carboxylic acid. To a stirred solution of ethyl 1-(6-((tert-butoxycarbonyl)amino) hexyl)-1H-indole-4-carboxylate (0.55 g, 1.41 mmol) in a mixture of THF (5 mL), MeOH (5 mL) and water (2 mL) was added LiOH.H₂O (0.12 g, 2.83 mmol) and the reaction mixture was stirred at rt for 1 hr. TAfter completion of reaction, the reaction mixture was concentrated under reduced pressure. The crude material was dissolved in water, and acidified with 1N HCl to pH 3. The precipitated solid was filtered, washed with water, diethyl ether, and hexane and dried in vacuo to afford the title compound (0.40 g, 78% yield) as an off white solid. 1H NMR (400 MHZ, DMSO-d₆) δ 12.60 (brs, 1H), 7.71-7.77 (m, 2H), 7.52 (d, J=2.93 Hz, 1H), 7.18-7.24 (m, 1H), 6.94 (d, J=2.93 Hz, 1H), 6.74 (t, J=4.89 Hz, 1H), 4.21 (t, J=7.09 Hz, 2H), 2.86 (q, J-6.20 Hz, 2H), 1.69-1.78 (m, 2H), 1.36 (s, 9H), 1.27-1.33 (m, 2H), 1.18-1.27 (m, 4H); LC-MS: m/z 261.25 [M-boc+H]+.

### Example 1 (Method 1): Synthesis of 2-[2-Oxo-5-[(3S)-1-(3-pyridyl) piperidine-3-carbonyl]amino]-1-pyridyl] acetic acid (I-91)

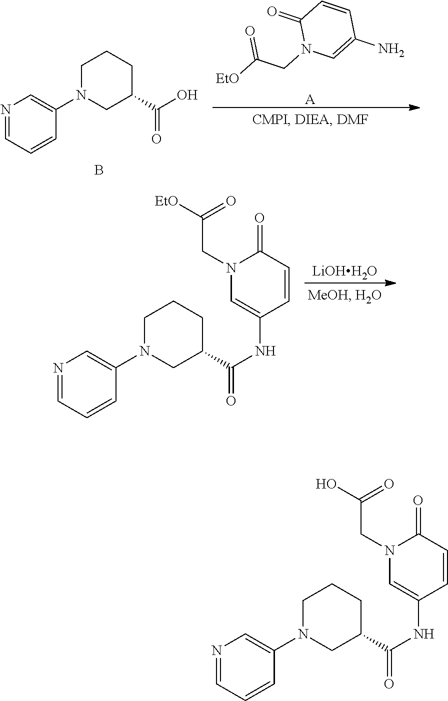

Step 1-Ethyl 2-[2-oxo-5-[[(3S)-1-(3-pyridyl) piperidine-3-carbonyl]amino]-1-pyridyl] acetate. To a solution of (3S)-1-(3-pyridyl) piperidine-3-carboxylic acid (300 mg, 1.45 mmol, Intermediate B) in DMF (2 mL) was added DIEA (752 mg, 5.82 mmol) and CMPI (446 mg, 1.75 mmol), and the mixture was stirred at 0° C. for 15 mins. Then ethyl 2-(5-amino-2-oxo-1-pyridyl)acetate (285 mg, 1.45 mmol, Intermediate A) was added, and the mixture was stirred at 0° C. for an additional 15 mins. On completion, the reaction mixture was concentrated in vacuo to give a residue. The residue was purified by prep-HPLC (column: Waters xbridge 150×25 mm 10 um; mobile phase: [water (NH₄HCO3)-ACN]) to give the title compound (80.0 mg, 13% yield, 100% ee) as brown solid. 1H NMR (400 MHZ, DMSO-d₆) δ 9.79 (s, 1H), 8.33 (s, 1H), 8.17 (s, 1H), 7.96 (d, J=4.4 Hz, 1H), 7.48-7.42 (m, 1H), 7.37-7.32 (m, 1H), 7.22-7.17 (m, 1H), 6.45-6.40 (m, 1H), 4.69 (s, 2H), 4.18-4.10 (m, 2H), 3.88 (d, J=12.0 Hz, 1H), 3.74 (d, J=13.2 Hz, 1H), 2.87 (t, J=11.6 Hz, 1H), 2.75 (t, J=11.6 Hz, 1H), 2.61 (t, J=10.4 Hz, 1H), 1.93 (d, J=11.2 Hz, 1H), 1.75 (d, J=11.2 Hz, 1H), 1.70-1.50 (m, 2H), 1.24-1.18 (m, 3H); LC-MS (ESI+) m/z 385.3 (M+H)+.

Step 2-2-[2-Oxo-5-[[(3S)-1-(3-pyridyl) piperidine-3-carbonyl]amino]-1-pyridyl] acetic acid. To a solution of ethyl 2-[2-oxo-5-[(3S)-1-(3-pyridyl) piperidine-3-carbonyl]amino]-1-pyridyl] acetate (80.0 mg, 208 umol) in MeOH (1 mL) and H₂O (0.215 mL) was added LiOH.H₂O (34.9 mg, 832 umol), then the mixture was stirred at 25° C. for 30 mins. On completion, the reaction mixture was concentrated in vacuo to give a residue. The residue was diluted with H₂O (1 mL) and HCl (4 M) was added to adjust the pH to 6. The mixture was then purified by prep-HPLC (column: Welch Xtimate C18 150×25 mm×5 um; mobile phase: [water (NH₄HCO3)-ACN]) to give the title compound (39.3 mg, 53% yield, 97% ee) as yellow solid.

1H NMR (400 MHZ, DMSO-d₆) δ 9.77 (s, 1H), 8.33 (d, J=2.8 Hz, 1H), 8.05 (d, J=2.4 Hz, 1H), 7.96 (d, J=4.4 Hz, 1H), 7.43-7.37 (m, 1H), 7.35-7.28 (m, 1H), 7.20-7.09 (m, 1H), 6.38 (d, J=9.6 Hz, 1H), 4.49 (s, 2H), 3.88 (d, J=12.0 Hz, 1H), 3.74 (d, J=12.8 Hz, 1H), 2.87 (t, J=11.6 Hz, 1H), 2.79-2.71 (m, 1H), 2.64-2.56 (m, 1H), 2.53-2.52 (m, 1H), 1.96-1.90 (m, 1H), 1.75 (d, J=12.0 Hz, 1H), 1.70-1.50 (m, 2H); LC-MS (ESI+) m/z 357.2 (M+H)*.

TABLE 4
Compounds synthesized via Method 1 using the corresponding amines and acids for the coupling.

| | | | LCMS | |
|---|---|---|---|---|
| | | | (ESI+) | |
| | | | m/z | |
| I-#^{a} | Amine | Acid | (M + H)⁺ | ¹H NMR (400 MHz, DMSO-d6) |
| I-9 | A | M | 437.0 | 13.25-12.58 (m, 1H), 9.79 (s, 1H), 9.20 (s, 1H), 8.22 (s, 1H), |
| | | | | 8.15-8.11 (m, 1H), 7.73-7.65 (m, 1H), 7.62-7.57 (m, 1H), 7.43 (dd, |
| | | | | J = 2.8, 9.6 Hz, 1H), 7.10 (d, J = 7.6 Hz, 1H), 6.41 (d, J = 9.6 Hz, |
| | | | | 1H), 4.59 (s, 2H), 4.00 (s, 3H), 3.46 (d, J = 10.4 Hz, 1H), |
| | | | | 3.38-3.35 (m, 1H), 2.98 (t, J = 10.2 Hz, 1H), 2.90-2.72 (m, 2H), |
| | | | | 2.07-1.96(m, 1H), 1.94-1.77 (m, 2H), 1.74-1.58 (m, 1H) |
| I-39 | A | L | 385.1 | 9.79 (s, 1H), 8.14 (d, J = 2.8 Hz, 1H), 8.00 (d, J = 2.8 Hz, 1H), |
| | | | | 7.84 (d, J = 1.6 Hz, 1H), 7.41-7.33 (m, 1H), 7.20-7.18-7.10 (m, |
| | | | | 1H), 6.34 (d, J = 9.6 Hz, 1H), 4.36 (s, 2H), 3.90-3.84 (m, 1H), |
| | | | | 3.73 (d, J = 12.4 Hz, 1H), 2.88-2.79 (m, 2H), 2.70 (d, J = 12.0 |
| | | | | Hz, 1H), 2.55 (d, J = 7.6 Hz, 3H), 1.92 (d, J = 9.6 Hz, 1H), |
| | | | | 1.78-1.71 (m, 1H), 1.67-1.53 (m, 2H), 1.17 (t, J = 7.6 Hz, 3H)) |
| I-40^{b} | A | H | 386.1 | 9.77 (s, 1H), 8.13 (d, J = 2.8 Hz, 1H), 7.56 (d, J = 2.0 Hz, 1H), |
| | | | | 7.47-7.38 (m, 2H), 6.45-6.36 (m, 2H), 5.74-5.56 (m, 1H), 4.59 |
| | | | | (s, 2H), 3.77 (d, J = 10.4 Hz, 1H), 3.71-3.60 (m, 1H), 2.80 (t, J = |
| | | | | 11.6 Hz, 1H), 2.71 (d, J = 2.4 Hz, 1H), 2.67 (s, 3H), 2.63-2.56 |
| | | | | (m, 1H), 2.33 (s, 1H), 1.92 (d, J = 8.0 Hz, 1H), 1.78-1.70 (m, 1H), |
| | | | | 1.65-1.54 (m, 2H) |
| I-83 | C | B | 390.9 | 9.48 (s, 1H), 8.32 (d, J = 2.8 Hz, 1H), 8.00-7.95 (m, 1H), 7.80 (s, |
| | | | | 1H), 7.34 (dd, J = 1.6, 8.4 Hz, 1H), 7.21 (dd, J = 4.4, 8.4 Hz, 1H), |
| | | | | 6.61 (s, 1H), 4.44 (s, 2H), 3.84 (d, J = 12.4 Hz, 1H), 3.71 (d, J = |
| | | | | 12.4 Hz, 1H), 2.96-2.85 (m, 1H), 2.83-2.72 (m, 1H), 2.71-2.63 |
| | | | | (m, 1H), 1.95 (d, .J = 10.0 Hz, 1H), 1.80-1.72 (m, 1H), 1.72-1.50 |
| | | | | (m, 2H) |
| I-132^{c} | A | BI | 369.2 | 13.00 (brs, 1H), 10.26 (s, 1H), 8.40 (d, J = 4.89 Hz, 1H), 8.30 (brs, |
| | | | | 1H), 7.63-7.72 (m, 2H), 7.50 (d, J = 4.89 Hz, 1H), 6.80 (d, J = 3.42 |
| | | | | Hz, 1H), 6.47 (d, J = 9.78 Hz, 1H), 4.65 (brs, 2H), 4.12 (d, J = 7.34 |
| | | | | Hz, 2H), 2.18-2.26 (m, 1H), 0.85 (d, J-6.85 Hz, 6H) |
| I-137^{c, d} | A | BJ | 411.3 | 13.00 (brs, 1H), 9.99 (s, 1H), 8.30 (d, J = 1.47 Hz, 1H), 7.64-7.73 |
| | | | | (m, 3H), 7.60 (brs, 2H), 7.55 (d, J = 7.34 Hz, 1H), 7.50 (d, J = 2.93 |
| | | | | Hz, 1H), 7.25 (t, J = 7.83 Hz, 1H), 6.85 (d, J = 2.93 Hz, 1H), 6.47 (d, |
| | | | | J = 9.29 Hz, 1H), 4.66 (s, 2H), 4.23 (t, J = 6.60 Hz, 2H), 2.68-2.79 |
| | | | | (m, 2H), 1.70-1.80 (m, 2H), 1.42-1.53 (m, 2H), 1.28-1.37 (m, 2H), |
| | | | | 1.17-1.27 (m, 2H) |
| I-138^{c} | A | BG | 406.1 | 13.00 (brs, 1H), 10.37 (s, 1H), 8.64 (s, 1H), 8.30 (d, J-8.31 Hz, |
| | | | | 1H), 8.19-8.26 (m, 2H), 8.12 (d, J = 7.82 Hz, 2H), 7.67-7.79 (m, |
| | | | | 2H), 7.56-7.62 (m, 1H), 7.47-7.54 (m, 1H), 6.49 (d, J = 9.29 Hz, |
| | | | | 1H), 4.67 (s, 2H) |
| I-160^{c} | A | BJ | 509.4 | 13.00 (brs, 1H), 9.98 (s, 1H), 8.31 (d, J-2.45 Hz, 1H), 7.63-7.71 |
| | | | | (m, 2H), 7.48-7.56 (m, 2H), 7.24 (t, J = 7.58 Hz, 1H), 6.83 (d, |
| | | | | J = 2.93 Hz, 1H), 6.74 (brs, 1H), 6.46 (d, J = 9.78 Hz, 1H), 4.65 (s, |
| | | | | 2H), 4.21 (t, J = 6.85 Hz, 2H), 2.86 (q, J = 6.36 Hz, 2H), 1.69-1.79 |
| | | | | (m, 2H), 1.36 (s, 9H), 1.28-1.33 (m, 2H), 1.24 (brs, 4H) |

^{a}The amine acid coupling was run under standard coupling conditions with typical purification techniques for the final compound.. Step 1 was run for 1-12 hrs at 0° C. to rt. Step 2 was run for 0.5-3 hr from 0° C. to rt.
^{b}After the coupling, the BOC protecting group was removed with HCl/Dioxane in DCM at rt for 1 hr.
Then the ester was hydrolyzed as described in Step 2 of Method 1.
^{c}HATU was used in place of CMPI in Step 1.
^{d}The product of the hydrolysis after Step 2 was then deprotected with TFA in DCM at rt for 1 hr.

### Example 2 (Method 2): 2-[4-ethyl-2-oxo-5-[(3S)-1-(3-pyridyl) piperidine-3-carbonyl|amino]-1-pyridyl]acetic acid (I-50)

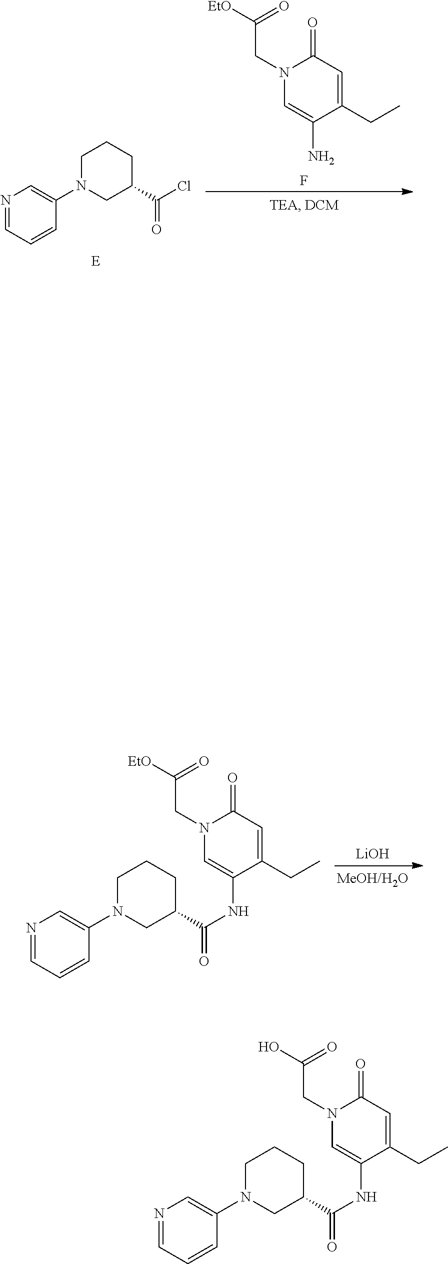

Step 1-Ethyl 2-[4-ethyl-2-oxo-5-[(3S)-1-(3-pyridyl) piperidine-3-carbonyl]amino]-1-pyridyl] acetate. A mixture of ethyl 2-(5-amino-4-ethyl-2-oxo-1-pyridyl)acetate (48.0 mg, 184 umol, HCl, Intermediate F) and TEA (93.1 mg, 920 umol) in DCM (5 mL) was added (3S)-1-(3-pyridyl) piperidine-3-carbonyl chloride (54.0 mg, 240 umol, Intermediate E) at 25° C. for 1 hr. On completion, the reaction mixture was quenched with water (0.2 mL) and concentrated in vacuo. The residue was purified by prep-HPLC (column: Phenomenex luna C18 150*25 mm*10 um; mobile phase: [water (FA)-ACN]; B %: 5%-35%, 10.5 min) to give the title compound (36 mg, 47% yield) as colorless oil. LC-MS (ESI+) m/z 413.1 (M+H)+.

Step 2 2-[4-Ethyl-2-oxo-5-[[(3S)-1-(3-pyridyl) piperidine-3-carbonyl]amino]-1-pyridyl] acetic acid. To a solution of ethyl 2-[4-ethyl-2-oxo-5-[[(3S)-1-(3-pyridyl) piperidine-3-carbonyl]amino]-1-pyridyl] acetate (36.0 mg, 87.2 umol) in MeOH (1 mL) and H₂O (0.3 mL) was added LiOH·H₂O (18.3 mg, 436 umol). The mixture was stirred at 25° C. for 1 hr. On completion, the reaction mixture was quenched with water (0.2 mL) and acidized with FA until the pH is 6. The mixture was purified by prep-HPLC (column: Phenomenex luna C18 150*25 mm*10 um; mobile phase: [water (FA)-ACN]; B %: 1%-30%, 10.5 min) to give the title compound (10.3 mg, 27% yield, FA) as white solid. 1H NMR (400 MHz, DMSO-d₆) δ 13.63-12.25 (m, 1H), 9.22 (s, 1H), 8.33 (d, J=2.8 Hz, 1H), 8.01-7.93 (m, 1H), 7.59 (s, IH), 7.39-7.30 (m, 1H), 7.25-7.17 (m, 1H), 6.23 (s, 1H), 4.54 (s, 2H), 3.89-3.78 (m, 1H), 3.72 (d, J=12.8 Hz, 1H), 2.95-2.87 (m, 1H), 2.81-2.74 (m, 1H), 2.65-2.61 (m, 1H), 2.41-2.33 (m, 2H), 2.01-1.88 (m, 1H), 1.81-1.71 (m, 1H), 1.70-1.53 (m, 2H), 1.12-1.03 (m, 3H); LC-MS (ESI+) m/z 385.1 (M+H)+.

TABLE 5
Compounds synthesized via Method 2 using the corresponding
amines and acyl chlorides for the coupling.

| | | | LCMS | |
|---|---|---|---|---|
| | | | (ESI+) | |
| | | Acyl | m/z | |
| I-#^{a} | Amine | Chloride | (M + H)⁺ | ¹H NMR (400 MHz, DMSO-d6) |
| I-41 | A | I | 407.0 | 9.80 (s, 1H), 9.01 (s, 1H), 8.20 (s, 1H), 8.13 (d, J = 2.4 Hz, |
| | | | | 1H), 8.09 (t, J = 8.4 Hz, 2H), 7.79 (t, J = 1.2, 7.6 Hz, 1H), |
| | | | | 7.70-7.65 (m, 1H), 7.44-7.36 (m, 1H), 6.41 (d, J = 9.6 Hz, 1H), |
| | | | | 4.59 (s, 2H), 3.48 (d, .J = 11.2 Hz, 1H), 3.04-2.95 (m, 1H), |
| | | | | 2.92-2.75 (m, 2H), 2.52 (d, J = 1.6 Hz, 1H), 2.08-1.98 (m, |
| | | | | 1H), 1.95-1.80 (m, 2H), 1.74-1.60 (m, 1H) |
| I-45 | A | K | 387.0 | 9.78 (s, 1H), 8.14 (d, J = 2.4 Hz, 1H), 7.95 (d, J = 2.4 Hz, 1H), |
| | | | | 7.70 (d, J = 2.4 Hz, 1H), 7.45 (dd, J = 2.8, 9.6 Hz, 1H), 6.88 |
| | | | | (t, J = 2.4 Hz, 1H), 6.42 (d, J = 9.6 Hz, 1H), 4.61 (s, 2H), 3.90 |
| | | | | (d, J = 12.4Hz, 1H), 3.80 (s, 3H), 3.75 (s, 1H), 2.93-2.84 (m, |
| | | | | 1H), 2.82-2.71 (m, 1H), 2.60 (m, J = 3.6, 7.2, 10.8 Hz, 1H), |
| | | | | 1.93 (d, J = 10.8 Hz, 1H), 1.79-1.70 (m, 1H), 1.70-1.49 (m, |
| | | | | 2H) |
| I-47 | A | J | 379.9 | 10.18 (s, 1H), 8.59 (d, J = 1.6 Hz, 1H), 8.34 (d, J = 2.8 Hz, |
| | | | | 1H), 8.27 (s, 1H), 8.22 (d, J = 2.4 Hz, 1H), 7.97 (t, J = 7.2 Hz, |
| | | | | 2H), 7.73 (t, J = 2.4 Hz, 1H), 7.70-7.63 (m, 2H), 6.48 (d, J = |
| | | | | 9.6 Hz, 1H), 4.65 (s, 2H), 3.93 (s, 3H) |

^{a}The coupling was run under standard conditions with typical purification techniques for the final compound.. DIEA in THF, with or without molecular sieves, could also be used for the coupling in Step 1. Step 1 was run for 1-2 h at rt; Step 2 was run for 0.5 to 2 hr at rt.

### Example 3 (Method 3): Synthesis of 2-(5-(N-(8-(2-((S)-4-(4-chlorophenyl)-2,3,9-trimethyl-6H-thieno[3,2-f][1,2,4]triazolo[4,3-a][1,4]diazepin-6-yl) acetamido) octyl)-1-(isoquinolin-4-yl) piperidine-3-carboxamido)-2-oxopyridin-1 (2H)-yl) acetic acid (I-478)

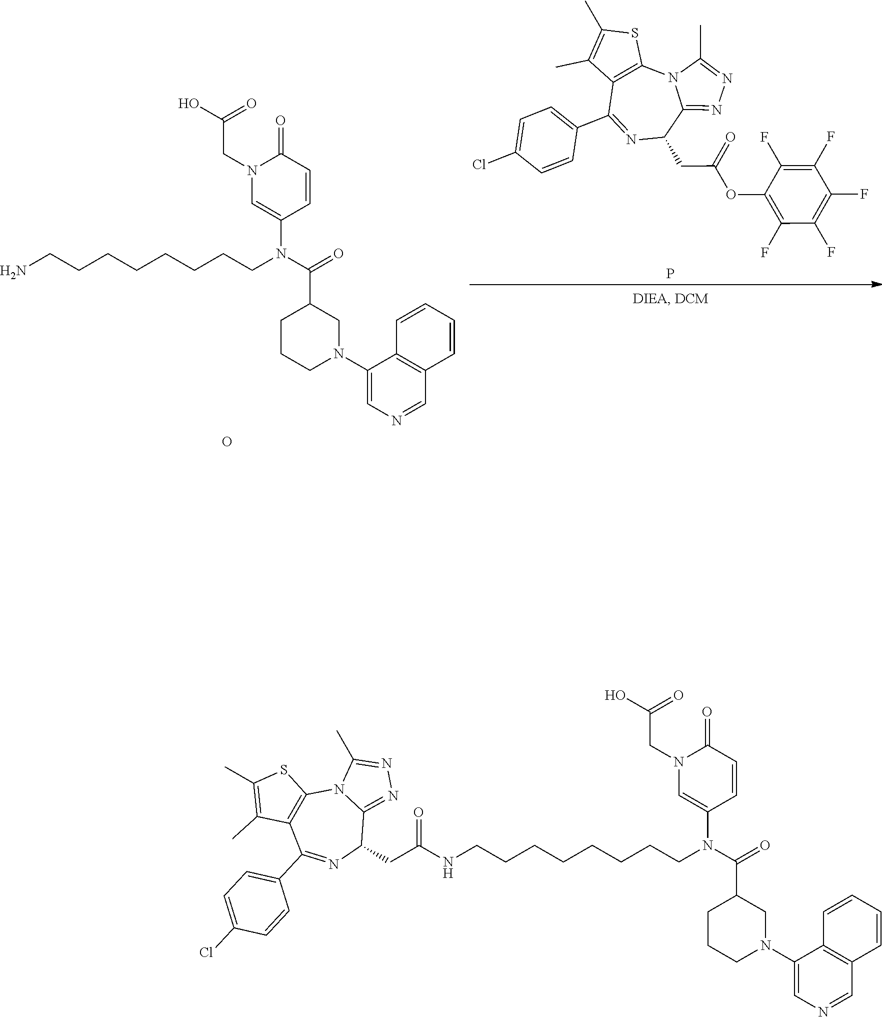

To a stirred mixture of {5-[N-(8-aminooctyl)₁-(isoquinolin-4-yl) piperidine-3-amido]-2-oxopyridin-1-yl} acctic acid (50 mg, 0.094 mmol, Intermediate O) and 2,3,4,5,6-pentafluorophenyl 2-[(9S)-7-(4-chlorophenyl)-4,5,13-trimethyl-3-thia-1,8,11,12-tetraazatricyclo [8.3.0.0∧{2,6}] trideca-2 (6),4,7,10,12-pentaen-9-yl] acetate (37.56 mg, 0.094 mmol, Intermediate P) in DCM (2 mL) was added DIEA (36.33 mg, 0.282 mmol) at rt. The resulting mixture was stirred for 16 h at rt under nitrogen atmosphere. On completion, the mixture was concentrated under vacuum. The residue was dissolved in DMSO (2 mL) and the solution was purified by reverse phase flash chromatography (Column: WelFlash™ C18-I, 20-40 um, 330 g; Eluent A: Water (plus 10 mmol/L NH₄HCO3); Eluent B: ACN; Gradient: 20%-50% B in 30 min; Flow rate: 80 mL/min; Detector: 220/254 nm; desired fractions were collected at 38% B) and concentrated under reduced pressure to afford the title compound (13.7 mg, 16% yield) as an off-white solid. LC/MS (ESI, m/z): [(M+1)]+=916.4; 1H NMR (400 MHZ, DMSO-d₆) δ 8.98 (s, 1H), 8.18-8.11 (m, 2H), 8.07 (d, J=8.3 Hz, 1H), 7.91-7.86 (m, 1H), 7.82-7.75 (m, 1H), 7.74-7.63 (m, 2H), 7.50-7.39 (m, 5H), 6.51 (d, J=9.7 Hz, 1H), 4.74-4.60 (m, 1H), 4.50 (dd, J=8.2, 6.0 Hz, 2H), 3.22-3.15 (m, 3H), 3.11-2.98 (m, 4H), 2.92-2.71 (m, 4H), 2.59 (s, 3H), 2.41 (s, 3H), 1.94-1.83 (m, 1H), 1.81-1.73 (m, 1H), 1.67-1.55 (m, 5H), 1.48-1.31 (m, 4H), 1.31-1.12 (m, 8H).

TABLE 6
Compounds synthesized via Method 3 using the corresponding
amines and activated ester for the coupling.

| | | | LCMS | |
|---|---|---|---|---|
| | | | (ESI+) | |
| | | | m/z | |
| I-#^{a} | Amine | Ester | (M + H)⁺ | ¹H NMR (400 MHz, DMSO-d6) |
| I-479 | U | P | 958.3 | 8.98 (s, 1H), 8.21-8.11 (m, 2H), 8.07 (d, J = 8.0 Hz, 1H), 7.89 (d, |
| | | | | J = 2.8 Hz, 1H), 7.78 (d, J = 8.5 Hz, 1H), 7.72-7.61 (m, 2H), |
| | | | | 7.53-7.38 (m, 5H), 6.52 (d, J = 9.6 Hz, 1H). 4.76-4.46 (m, 2H), |
| | | | | 3.29-2.98 (m, 5H), 2.92-2.73 (m, 3H), 2.60 (s, 3H), 2.59-2.53 (m, 2H), |
| | | | | 2.41 (s, 3H), 1.94-1.83(m, 1H), 1.81-1.73 (m, 2H), 1.68-1.57 (m, |
| | | | | 4H), 1.47-1.35 (m, 4H), 1.28-1.16 (m, 16H) |
| I-480^{b} | R | P | 962.1 | 9.05 (s, 1H), 8.15 (s, 1H), 7.98 (d, J = 9.4 Hz, 2H), 7.87-7.74 (m, |
| | | | | 2H), 7.65 (t, J = 6.9 Hz, 1H), 7.54-7.42 (m, 3H), 7.38-7.27 (m, |
| | | | | 3H), 6.63 (d, J = 9.6 Hz, 1H). 4.73-4.63 (m, 2H), 3.75-3.40 (m, |
| | | | | 14H), 3.09-2.88 (m, 3H), 2.69-2.61 (m, 5H), 2.50-2.43 (m, 5H), |
| | | | | 2.03-1.95 (m, 2H), 1.89-1.78 (m, 3H), 1.78-1.66 (m, 4H), 1.54-1.38 |
| | | | | (m, 1H) |
| I-481 | X | P | 1000.5 | 8.98 (s, 1H), 8.17 (t, J = 5.7 Hz, 1H), 8.12 (s, 1H), 8.07 (d, J = 8.0 |
| | | | | Hz, 1H), 7.86-7.82 (m, 1H), 7.80-7.75 (m, 1H), 7.74-7.69 (m, 1H), |
| | | | | 7.68-7.62( m, 1H), 7.51-7.39 (m, 5H), 6.49 (d, J = 9.6 Hz, 1H), |
| | | | | 4.71-4.57 (m, 1H), 4.50 (dd, J = 8.5, 5.6 Hz, 2H), 3.26-3.22 (m, |
| | | | | 2H), 3.18-3.09 (m, 4H), 3.09-2.99 (m, 1H), 2.94-2.84 (m, 2H), |
| | | | | 2.84-2.72 (m, 2H), 2.59 (s, 3H), 2.41 (s, 3H), 1.93-1.83 (m, 1H), |
| | | | | 1.79-1.72 (m, 1H), 1.70-1.64 (m, 1H), 1.62 (s, 3H), 1.60-1.52 |
| | | | | (m, 1H), 1.47-1.33 (m, 4H), 1.32-1.09 (m, 20H) |
| I-451^{c} | AS | AT | 1256.5 | 12.17 (s, 1H), 10.18 (s, 1H), 8.82-8.81 (m, 2H), 8.44-8.42 (m, 2H), |
| | | | | 8.34-8.32 (m, 1H), 8.29-8.20 (m, 3H), 8.03-8.01 (m, 1H), 8.00- |
| | | | | 7.91 (m, 3H), 7.72-7.61 (m, 2H), 7.57-7.53 (m, 1H), 7.46-7.42 (m, |
| | | | | 1H), 7.29-7.24 (m, 2H), 7.18-7.14 (m, 3H), 6.79-6.74 (m, 1H), |
| | | | | 6.70 (s, 1H), 6.50-6.46 (m, 1H), 4.84-4.75 (m, 1H), 4.67 (s, 2H), |
| | | | | 4.53-4.44 (m, 2H), 4.29-4.22 (m, 3H), 4.16-4.06 (m, 2H), 4.00- |
| | | | | 3.92 (m, 2H), 3.87-3.79 (m, 1H), 3.20-3.15 (m, 2H), 3.05-2.97 (m, |
| | | | | 2H), 2.88-2.81 (m, 1H), 2.70-2.66 (m, 2H), 2.27-2.15 (m, 2H), |
| | | | | 2.14-2.11 (m, 2H), 2.01-1.93 (m, 3H), 1.92-1.78 (m, 2H), 1.69-1.64 |
| | | | | (m, 3H), 1.58-1.47 (m, 5H), 1.34-1.30 (m, 9H), 1.19-1.15 (m, |
| | | | | 6H) |

^{a}The coupling was run with standard conditions with typical purification techniques for the final compound. DMA could also be employed as the solvent. The reaction was run for 1-16 hr at rt.
^{b}LCMS data reported as the (M-H)⁻ ion.
^{c}NMP and TEA was used for the coupling at rt for 1 hr.

### Example 4 (Method 4): Synthesis of 2-(5-((R)—N-(1-((5R,8R,10aS)-8-(((R)-5-amino-1-(benzhydrylamino)-1,5-dioxopentan-2-yl) carbamoyl)-5-(5-((hydroxyoxidophosphoryl) carbonyl)-1H-indole-2-carboxamido)-6-oxooctahydropyrrolo[1,2-a][1,5] diazocin-3 (4//)-yl)-1-oxo-5,8,11-trioxa-2-azatridecan-13-yl)-1-(isoquinolin-4-yl) piperidine-3-carboxamido)-2-oxopyridin-1 (2H)-yl)acetate (I-483)

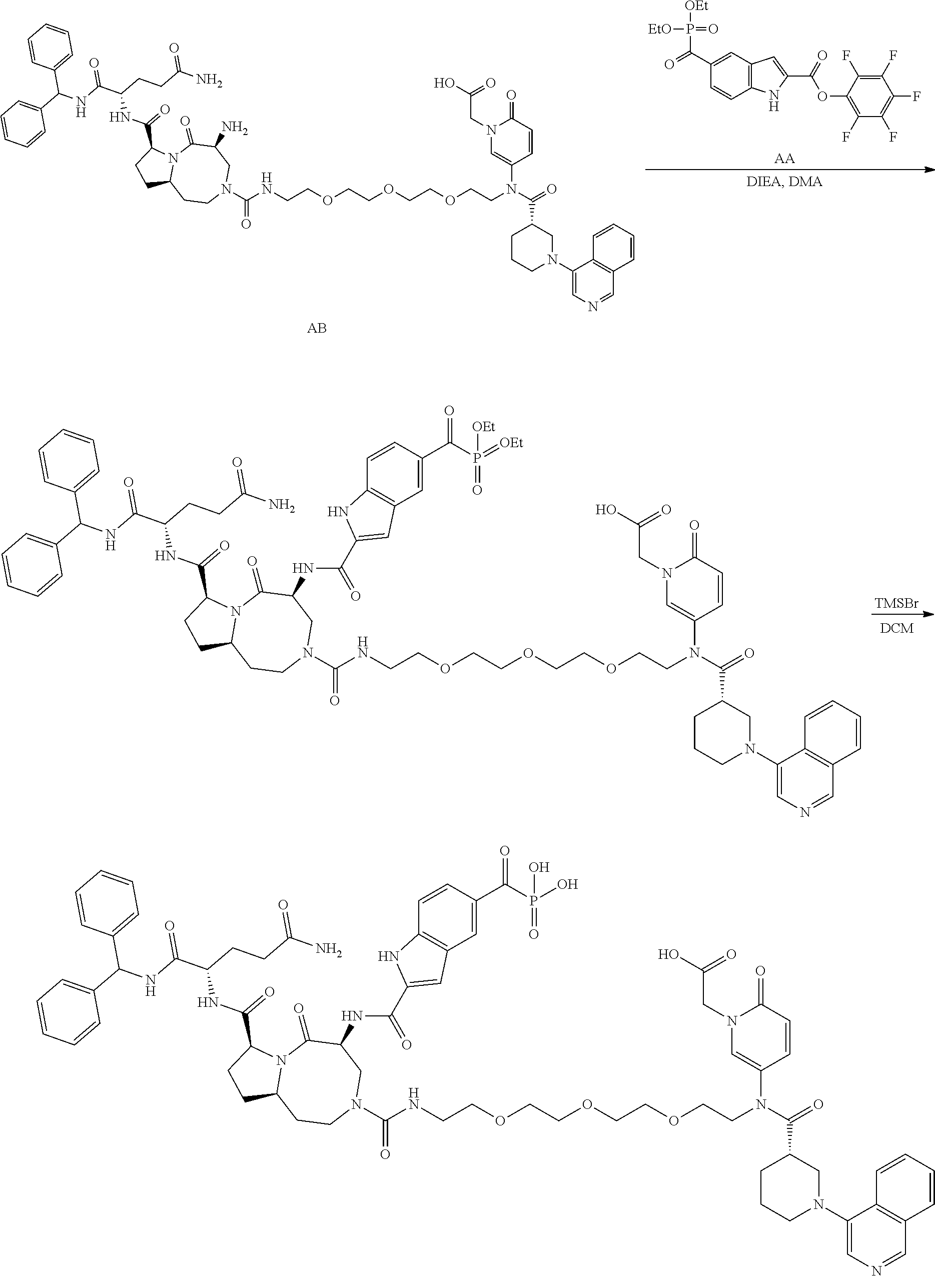

Step 1-2-(5-((S)—N-(1-((5S,8S,10aR)-8-(((S)-5-amino-1-(benzhydrylamino)-1,5-dioxopentan-2-yl) carbamoyl)-5-(5-((diethoxyphosphoryl) carbonyl)-1H-indole-2-carboxamido)-6-oxooctahydropyrrolo[1,2-a][1,5] diazocin-3 (4H)-yl)-1-oxo-5,8,11-trioxa-2-azatridecan-13-yl)-1-(isoquinolin-4-yl) piperidine-3-carboxamido)-2-oxopyridin-1 (2H)-yl) acetic acid. To a stirred mixture of 2-(5-((S)—N-(1-((5S,8S,10aR)-5-amino-8-(((S)-5-amino-1-(benzhydrylamino)-1,5-dioxopentan-2-yl) carbamoyl)-6-oxooctahydropyrrolo[1,2-a][1,5] diazocin-3 (4H)-yl)-1-oxo-5,8,11-trioxa-2-azatridecan-13-yl)-1-(isoquinolin-4-yl) piperidine-3-carboxamido)-2-oxopyridin-1 (2H)-yl) acetic acid (180 mg, 0.16 mmol, Intermediate AB) in DMA (4 mL) was added DIEA (103 mg, 0.80 mmol) dropwise at rt. The resulting mixture was stirred for 10 min at rt. Then, perfluorophenyl 5-((diethoxyphosphoryl) carbonyl)-IH-indole-2-carboxylate (157 mg, 0.32 mmol, Intermediate AA) was added to the mixture at rt and the mixture was stirred for additional 1 hr at rt. On completion, the reaction mixture was purified by reversed-phase flash chromatography (Column: Spherical C18, 20˜40 μm, 120 g; Mobile Phase A: Water (plus 0.1% HCOOH), Mobile Phase B: acetonitrile; Flow rate: 50 mL/min; Gradient (B %): 5%˜22%, 4 min; 22% ˜40%, 20 min; 40% ˜95%; 2 min; 95%, 5 min; Detector: 254 nm; the fractions containing desired product were collected at 31% B) and concentrated under reduced pressure to afford the title compound (120 mg, 53% yield) as a light yellow solid. 1H NMR (400 MHZ, DMSO-d₆) § 12.33 (s, 1H), 8.98 (s, 1H), 8.79 (d, J=8.4 Hz, 1H), 8.74 (s, 1H), 8.48 (s, 1H), 8.27 (d, J=8.0 Hz, 1H), 8.13 (s, 1H), 8.07 (d, J=8.0 Hz, 1H), 7.95 (d, J=8.8 Hz, 1H), 7.89 (s, 1H), 7.82-7.45 (m, 4H), 7.38-7.19 (m, 11l H), 6.77-6.71 (m, 2H), 6.54-6.48 (m, 1H), 6.11 (d, J=8.4 Hz, 1H), 4.83-4.78 (m, 1H), 4.61-4.55 (m, 1H), 4.47 (t, J=8.4 Hz, 1H), 4.38 (q, J=7.6 Hz, 1H), 4.29-4.06 (m, 6H), 4.02-3.98 (m, 1H), 3.84-3.79 (m, 1H), 3.67-3.43 (m, 16l H), 3.37-3.16 (m, 12l H), 3.06-2.99 (m, 1H), 2.94--2.88 (m, 1H), 2.82-2.77 (m, 2H), 2.24-2.08 (m, 1H), 2.03-1.86 (m, 2H), 1.83-1.75 (m, 1H), 1.72-1.48 (m, 4H), 1.29 (t, J=7.2 Hz, 6H); LC/MS (ESI, m/z): [(M+1)]+=1435.8.

Step 2-Ammonium rac-2-(5-((R)—N-(1-((5R,8R,10aS)-8-(((R)-5-amino-1-(benzhydrylamino)-1,5-dioxopentan-2-yl) carbamoyl)-5-(5-((hydroxyoxidophosphoryl) carbonyl)-1H-indole-2-carboxamido)-6-oxooctahydropyrrolo[1,2-a][1,5] diazocin-3 (4H)-yl)-1-oxo-5,8, 11-trioxa-2-azatridecan-13-yl)-1-(isoquinolin-4-yl) piperidine-3-carboxamido)-2-oxopyridin-1 (2H)-yl)acetate. To a stirred mixture of 2-(5-((S)—N-(1-((55,8S,10aR)-8-(((S)-5-amino-1-(benzhydrylamino)-1,5-dioxopentan-2-yl) carbamoyl)-5-(5-((diethoxyphosphoryl) carbonyl)-1H-indole-2-carboxamido)-6-oxooctahydropyrrolo[1,2-a][1,5] diazocin-3 (4H)-yl)-1-oxo-5,8,11-trioxa-2-azatridecan-13-yl)-1-(isoquinolin-4-yl) piperidine-3-carboxamido)-2-oxopyridin-1 (2H)-yl) acetic acid (90 mg, 0.06 mmol) in DCM (2 mL) was added bromotrimethylsilane (0.4 mL) dropwise at rt. The resulting mixture was stirred for 2 h at 50° C. On completion, the mixture was cooled to rt and purified by reversed-phase flash chromatography (Column: Spherical C18, 20˜40 μm, 120 g; Mobile Phase A: Water (plus 10 mM NH₄HCO3), Mobile Phase B: acetonitrile; Flow rate: 50 mL/min; Gradient (B %): 5%˜22%, 4 min; 22% ˜40%, 20 min; 40% ˜95%; 2 min; 95%, 5 min; Detector: 254 nm; the fractions containing desired product were collected at 26% B) and concentrated under reduced pressure to afford the title compound (67 mg, 78% yield) as a light yellow solid. 1H NMR (400 MHZ, DMSO-d₆) δ 12.15 (s, 1H), 8.97 (s, 1H), 8.90-8.86 (m, 2H), 8.55-8.40 (m, 2H), 8.12 (s, 1H), 8.06 (d, J=8.0 Hz, 1H), 7.99-7.90 (m, 1H), 7.79-7.70 (m, 3H), 7.67-7.62 (m, 1H), 7.51-7.41 (m, 2H), 7.36-7.19 (m, 12l H), 6.90-6.84 (m, 1H), 6.76 (s, 1H), 6.49-6.42 (m, 1H), 6.11 (d, J=8.4 Hz, 1H), 4.79 (t, J=8.8 Hz, 1H), 4.61-4.44 (m, 2H), 4.39-4.33 (m, 1H), 4.14-4.08 (m, 1H), 3.98-3.94 (m, 1H), 3.87-3.77 (m, 1H), 3.69-3.53 (m, 2H), 3.49-3.35 (m, 14l H), 3.34-3.14 (m, 6H), 3.07-3.00 (m, 1H), 2.95-2.88 (m, 1H), 2.82-2.75 (m, 2H), 2.25-1.98 (m, 3H), 1.98-1.81 (m, 4H), 1.80-1.44 (m, 5H). LC/MS (ESI, m/z): [(M+1)]+=1379.6.

TABLE 7
Compounds synthesized via Method 4 using the corresponding
amines and activated ester for the coupling in Step 1.

| | | | LCMS | |
|---|---|---|---|---|
| | | | (ESI+) | |
| | | | m/z | |
| I-#^{a} | Amine | Acid | (M + H)⁺ | ¹H NMR (400 MHz, DMSO-d6) |
| I-482 | AF | AA | 1373.5 | 12.05 (s, 1H), 8.98 (s, 1H), 8.88 (s, 1H), 8.81 (d, J = 8.4 Hz, 1H), |
| | | | | 8.39-8.28 (m, 2H), 8.12 (s, 1H), 8.07 (d, J = 8.0 Hz, 1H), 7.98 (d, |
| | | | | J = 8.8 Hz, 1H), 7.86 (s, 1H), 7.81-7.61 (m, 4H), 7.55-7.40 (m, |
| | | | | 3H), 7.40-7.17 (m, 16H), 6.85-6.74 (m, 2H), 6.51 (d, J = 9.6 Hz, |
| | | | | 1H), 6.11 (d, J = 8.4 Hz, 1H), 4.85-4.61 (m, 2H), 4.50 (t, J = 8.8 |
| | | | | Hz, 1H), 4.44-4.32 (m, 1H), 4.18-4.02 (m, 1H), 4.01-3.78 (m, 2H), |
| | | | | 3.34-3.08 (m, 6H), 3.06-2.96 (m, 1H), 2.95-2.71 (m, 3H), 2.29- |
| | | | | 2.07 (m, 2H), 2.05-1.94 (m, 2H), 1.94-1.84 (m, 2H), 1.81-1.71 (m, |
| | | | | 2H), 1.70-1.59 (m, 2H), 1.58-1.44 (m, 3H), 1.41-1.05 (m, 21H) |
| I-484 | AJ | AA | 1423.7 | 12.06 (s, 1H), 8.98 (s, 1H), 8.89 (s, 1H), 8.78 (d, J = 8.4 Hz, 1H), |
| | | | | 8.46-8.34 (m, 1H), 8.30 (d, J = 7.6 Hz, 1H), 8.12 (s, 1H), 8.06 (d, |
| | | | | J = 8.0 Hz, 1H), 7.97 (d, J = 8.8 Hz, 1H), 7.87-7.74 (m, 2H), |
| | | | | 7.75-7.59 (m, 1H), 7.58-7.49 (m, 1H), 7.44 (d, J = 8.8 Hz, 1H), |
| | | | | 7.38-7.13 (m, 14H), 6.92-6.66 (m, 2H), 6.57-6.43 (m, 1H), 6.11 (d, J = |
| | | | | 8.4 Hz, 1H), 4.86-4.61 (m, 2H), 4.50 (t, J = 8.8 Hz, 2H), 4.38 (q, J |
| | | | | = 7.6 Hz, 1H), 4.18-4.05 (m, 1H), 4.02-3.90 (m, 1H), 3.89-3.77 (m, |
| | | | | 1H), 3.73-3.63 (m, 3H), 3.58-3.34 (m, 13H), 3.35-3.14 (m, 7H), |
| | | | | 3.11-2.98 (m, 2H), 2.96-2.85 (m, 1H), 2.85-2.73 (m, 2H), 2.25-1.97 |
| | | | | (m, 4H), 1.98-1.83 (m, 3H), 1.83-1.72 (m, 2H), 1.71-1.46 (m, |
| | | | | 4H) |
| 1-485 | AM | AA | 1415.2 | 12.04 (s, 1H), 8.97 (s, 1H), 8.90-8.76 (m, 2H), 8.46-8.31 (m, 2H), |
| | | | | 8.18-7.95 (m, 3H), 7.86-7.63 (m, 5H), 7.54-7.43 (m, 2H), 7.38- |
| | | | | 7.17 (m, 13H), 6.79 (s, 2H), 6.49 (d, J = 9.6 Hz, 1H), 6.11 (d, J = |
| | | | | 8.4 Hz, 1H), 4.83-4.75 (m, 1H), 4.72-4.61 (m, 1H), 4.50 (t, J = 8.8 |
| | | | | Hz, 2H), 4.38 (q, J = 7.6 Hz, 1H), 4.17-4.04 (m, 1H), 4.02-3.92 (m, |
| | | | | 1H), 3.91-3.80 (m, 1H), 3.70-3.44 (m, 2H), 3.34-3.08 (m, 6H), |
| | | | | 3.06-2.96 (m, 1H), 2.95-2.85 (m, 1H), 2.83-2.72 (m, 3H), 2.27-2.08 |
| | | | | (m, 2H), 2.06-1.82 (m, 3H), 1.80-1.71 (m, 2H), 1.69-1.60 (m, |
| | | | | 2H), 1.60-1.46 (m, 3H), 1.44-1.07 (m, 23H) |

^{a}The coupling was run with standard conditions with typical purification techniques for the final compound. Step 1 was run for 1-2 hrs at rt. Step 2 was run for 2-16 hr at 50° C.

### Example 5 (Method 5): 2-(2-oxo-5-(3-(thiophen-2-yl)benzamido) pyridin-1 (2H)-yl) acetic acid (I-172)

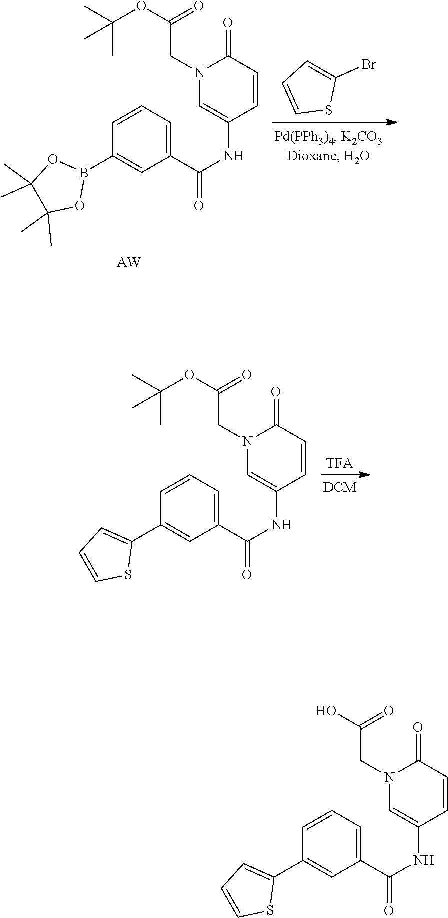

Step 1-Tert-butyl 2-(2-oxo-5-(3-(thiophen-2-yl)benzamido)pyridin-1 (2H)-yl)acetate. To a stirred solution of 2-bromothiophene (0.06 g, 0.35 mmol, Intermediate AX) and tert-butyl 2-(2-oxo-5-(3-(4,4,5,5-tetramethyl-1,3,2-dioxaborolan-2-yl)benzamido)pyridin-1 (2H)-yl)acetate (0.15 g, 0.35 mmol, Intermediate AW) in 1,4 dioxane (3 mL) and H₂O (1 mL), was added K₂CO₃ (0.12 g, 0.88 mmol) and the reaction mixture was purged with argon for 20 min. To the resulting reaction mixture was added Pd (PPh3)₄ (0.04 g, 0.04 mmol) and the reaction mixture was again purged with argon for 20 min. The reaction mixture was then stirred at 90° C. for 3 h. After completion of reaction, the reaction mixture was filtered through celite, washed with ethyl acetate. The filtrate was diluted with water and the aqueous layer was extracted with ethyl acetate. The organic layer was washed with water and brine, dried over anhydrous Na₂SO₄ and concentrated under reduced pressure. The crude compound was purified by silica gel column chromatography (5% MeOH/DCM) to afford the title compound (0.05 g, 33%) as an off white solid. 1H NMR (400 MHZ, CDC13)δ 8.20 (brs, 1H), 8.06 (s, 1H), 7.80 (d, J=7.34 Hz, 1H), 7.69 (d, J-7.34 Hz, 1H), 7.59 (brs, 1H), 7.47-7.53 (m, 1H), 7.41 (d, J=3.91 Hz, 1H), 7.30-7.37 (m, 2H), 7.12 (dd, J=3.42, 4.89 Hz, 1H), 6.63 (d, J=9.78 Hz, 1H), 4.60 (s, 2H), 1.50 (s, 9H); LC-MS: m/z 354.90 [M-56+H]+.

Step 2-2-(2-oxo-5-(3-(thiophen-2-yl)benzamido)pyridin-1 (2H)-yl) acetic acid. To a stirred solution of tert-butyl 2-(2-oxo-5-(3-(thiophen-2-yl)benzamido)pyridin-1 (2H)-yl)acetate (0.05 g, 0.11 mmol) in DCM (2 mL) was added TFA (1.0 mL) at 0° C., then the reaction mixture was stirred at rt for 3 hr. After completion of reaction, the reaction mixture was concentrated under reduced pressure. The crude compound was triturated with diethyl ether followed by pentane, then filtered and dried in vacuo to afford the title compound (0.005 g, 14% yield) as an off white solid. 1H NMR (400 MHZ, DMSO-d₆) δ 13.05 (brs, 1H), 10.20 (s, 1H), 8.14-8.23 (m, 2H), 7.86 (t, J-9.05 Hz, 2H), 7.53-7.70 (m, 4H), 7.19 (t, J=4.16 Hz, 1H), 6.47 (d, J-9.78 Hz, 1H), 4.66 (s, 2H); LC-MS: m/z 355.10 [M+H]+.

TABLE 8
Compounds synthesized via Method 5 using the corresponding
bromides and boronic esters for the coupling in Step 1.

| | | | LCMS | |
|---|---|---|---|---|
| | | | (ESI+) | |
| | | Boronic | m/z | |
| I-#^{a} | Bromide | Ester | (M+H)⁺ | 1H NMR (400 MHz, DMSO-d6) |
| I-192 | BB | AW | 427.2 | 13.00 (brs, 1H), 10.21 (s, 1H), 8.29 (s, 1H), 8.22 (d, J = 2.45 |
| | | | | Hz, 1H), 8.02-8.08 (m, 4H), 7.99 (d, J = 7.83 Hz, 2H), 7.64-7.71 |
| | | | | (m, 2H), 6.48 (d, J = 9.78 Hz, 1H), 4.66 (s, 2H), 3.28 (s, |
| | | | | 3H) |
| I-193 | BA | AW | 406.2 | 13.01 (brs, 1H), 10.17-10.23 (m, 1H), 8.52 (d, J = 4.89 Hz, |
| | | | | 1H), 8.18-8.31 (m, 2H), 7.91-8.00 (m, 4H), 7.82-7.90 (m, |
| | | | | 2H), 7.61-7.71 (m, 2H), 6.44-6.51 (m, 1H), 4.66 (s, 2H), |
| | | | | 2.81 (d, J = 4.40 Hz, 3H) |
| I-194 | AZ | AW | 427.1 | 13.00 (brs, 1H), 10.23 (s, 1H), 8.27 (d, J = 6.36 Hz, 2H), 8.21 |
| | | | | (d, J-2.45 Hz, 1H), 8.10-8.16 (m, 1H), 7.95-8.02 (m, 3H), |
| | | | | 7.77-7.83 (m, 1H), 7.65-7.72 (m, 2H), 6.48 (d, J = 9.78 Hz, |
| | | | | 1H), 4.66 (s, 2H), 3.32 (s, 3H) |
| I-195 | AY | AW | 406.2 | 10.22 (brs, 1H), 8.62 (brs, 1H), 8.16-8.29 (m, 2H), 8.07 (s, |
| | | | | 1H), 7.80-7.98 (m, 4H), 7.54-7.73 (m, 3H), 6.34-6.43 (m, |
| | | | | 1H), 4.32-4.44 (m, 2H), 2.82 (d, J-3.91 Hz, 3H) |
| I-202^{b} | BG | BH | 364.0 | 10.36 (brs, 1H), 8.58-8.66 (m, 2H), 7.84-7.96 (m, 4H), |
| | | | | 7.43-7.65 (m, 4H), 6.29 (d, J-9.78 Hz, 1H), 4.13 (s, 2H), |
| | | | | 2.30 (s, 3H) |

^{a}The coupling was run with standard conditions with typical purification techniques for the final compound.
^{b}A boronic acid was used in Step 1, where the reaction was run at 100° C. for 8 hr. No Step 2 was required.

### Example 6 (Method 6): 2-(2-oxo-5-(3′-(trifluoromethyl)-[1,1′-biphenyl]-3-carboxamido)pyridin-1 (2H)-yl) acetic acid (I-200)

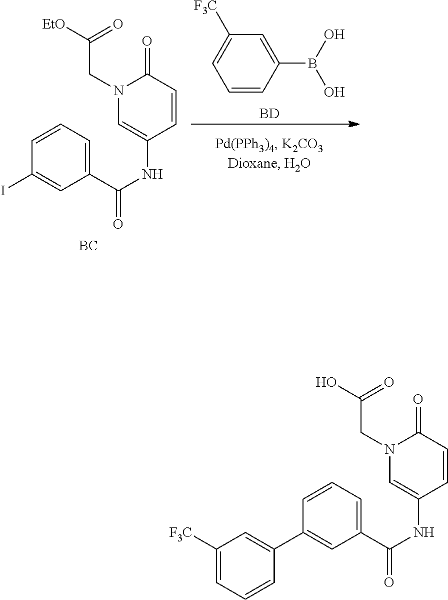

To a stirred solution of (3-(trifluoromethyl)phenyl) boronic acid (0.05 g, 0.25 mmol, Intermediate BD) and ethyl 2-(5-(3-iodobenzamido)-2-oxopyridin-1 (2H)-yl)acetate (0.10 g, 0.23 mmol, Intermediate BC) in 1,4 dioxane (10 mL) and H₂O (10 mL) was added K₂CO₃ (0.08 g, 0.58 mmol) and the reaction mixture was purged with argon for 15 min. To the resulting reaction mixture was added Pd (PPh3)₄ (0.03 g, 0.02 mmol) and the reaction mixture was again purged with argon for 15 min. The reaction mixture was then stirred at 90° C. for 6 hr. After completion of reaction, the reaction mixture was filtered through celite, and washed with 5% MeOH/DCM. The filtrate was concentrated under reduced pressure. The crude material was acidified with 1N HCl, then the aqueous layer was extracted with 10% MeOH/DCM. The combined organic layer was dried over anhydrous Na₂SO₄ and concentrated under reduced pressure. The crude compound was purified by prep HPLC to afford the title compound (0.024 g, 24% yield) as an off white solid. 1H NMR (400 MHZ, DMSO-d₆) δ 13.05 (brs, 1H), 10.20 (s, 1H), 8.17-8.33 (m, 2H), 8.09 (s, 2H), 7.91-8.03 (m, 2H), 7.72-7.84 (m, 2H), 7.63-7.71 (m, 2H), 6.48 (d, J=9.78 Hz, 1H), 4.66 (s, 2H); LC-MS: m/z 416.8 [M+H]+.

TABLE 9
Compounds synthesized via Method 6 using the corresponding
iodides and boronic acids for the coupling.

| | | | LCMS | |
|---|---|---|---|---|
| | | | (ESI+) | |
| | | Boronic | m/z | |
| I-#^{a} | Iodide | Acid | (M + H)⁺ | ¹H NMR (400 MHz, DMSO-d6) |
| I-199 | BC | BE | 416.8 | 13.05 (brs, 1H), 10.21 (s, 1H), 8.19-8.30 (m, 2H), 7.94-8.04 |
| | | | | (m, 4H), 7.88 (d, J = 8.31 Hz, 2H), 7.63-7.71 (m, 2H), 6.44-6.51 |
| | | | | (m, 1H), 4.66 (s, 2H) |
| I-201 | BC | BF | 350.9 | 10.18 (s, 1H), 9.25 (s, 3H), 8.34 (s, 1H), 8.21 (d, J = 2.45 Hz, |
| | | | | 1H), 7.99-8.07 (m, 2H), 7.64-7.75 (m, 2H), 6.48 (d, J = 9.29 |
| | | | | Hz, 1H), 4.64 (s, 2H) |

^{a}The coupling was run with standard conditions with typical purification techniques for the final compound.

### Example 7. KLHDC2 Binding Assays

HTRF binding assays were performed using 1 nM truncated His -MBP-Tev-KLHDC2 (residues 1-363) purified from E. coli, 0.2 nM terbium-anti-His antibody (CisBio), 1 nM fluorescein-labeled diglycine peptide probe, and test compounds in assay buffer consisting of 50 mM HEPES-Na pH 7.5, 100 mM NaCl, 2 mM DTT, 0.005% Tween-20 with a final volume of 20 uL. Compound stocks were dissolved at 10 mM in 100% DMSO and 11 point titration with 3 fold serial dilution was performed in white, opaque 384 well microplates. Reaction plates were incubated at room temperature for 30 minutes. Plates were centrifuged at low rpm for 5 mins, and the ratio of fluorescence intensities were measured at emission wavelengths for fluorescein acceptor (520 nm) and terbium donor (495 nm) on Envision Plate reader. % Inhibition was calculated from the 520/495 ratio generated by the no protein controls for 100% inhibition and DMSO only reactions for 0l % inhibition. Data was processed and dose response curves were generated using GraphPad Prism to determine the concentration required for inhibiting 50% of the HTRF signal (IC₅₀).

FP competition binding assays were performed using 15 nM truncated His -MBP-Tev-KLHDC2 (residues 1-363) purified from E. coli, 10 nM fluorescein-labeled diglycine peptide probe, and test compounds in assay buffer consisting of 50 mM HEPES-Na pH 7.5, 100 mM NaCl, 2 mM DTT, 0.005% Tween-20 with a final volume of 20 uL. Compound stocks were dissolved at 10 mM in 100% DMSO and 11 point titration with 3 fold serial dilution was performed in black, flat bottom 384 well microplates. Plates were incubated at room temperature for 30 minutes. Plates were centrifuged at 1000 rpm for 1 min, data were collected using an Envision plate reader in fluorescence polarization mode with excitation filter FITC FP at 480 nm, emission filter FITC FP P-pol at 535 nm and FITC FP S-pol at 535 nm. FP signal was calculated using the equation (mP=1000*(S-G*P)/(S+G*P), G-factor=1.0) and % Inhibition was calculated using no-protein controls for 100% inhibition and DMSO only reactions for 0l % inhibition (yes). Data was processed and dose response curves were generated using GraphPad Prism and fit to the equation (Y=Bottom+ (Top-Bottom)/(1+10∧ ((LogIC50−X)*HillSlope))) to determine the concentration required for inhibiting 50% of the FP signal (IC₅₀).

KLHDC2 HTRF and Human FP binding results for compounds of the invention are presented in Table 10. The letter codes for KLHDC2 IC₅₀ include: A (<0.05 μM), B (0.05-0.5 μM), C (>0.5-5 μM), D (>5.0 μM or not determinded).

TABLE 10
KLHDC2 HTRF and Human FP binding results

| | KLHDC2 | KLHDC2 |
|---|---|---|
| | binding | binding Human |
| | HTRF: | FP: average |
| I-# | average IC₅₀ | external IC₅₀ |
| I-1 | D | D |
| I-2 | C | D |
| 1-3 | C | B |
| I-4 | A | A |
| I-5 | B | B |
| I-6 | A | B |
| I-7 | A | B |
| 1-8 | A | A |
| I-9 | A | A |
| I-10 | C | D |
| I-11 | C | C |
| I-12 | D | D |
| I-13 | D | D |
| I-14 | D | D |
| I-15 | B | D |
| I-16 | D | D |
| I-17 | D | D |
| I-18 | B | C |
| I-19 | B | C |
| I-20 | B | C |
| I-21 | B | C |
| I-22 | D | D |
| I-23 | A | B |
| I-24 | B | B |
| I-25 | B | C |
| I-26 | B | C |
| I-27 | D | D |
| I-28 | C | D |
| I-29 | C | D |
| I-30 | D | D |
| I-31 | D | D |
| I-32 | C | D |
| I-33 | B | C |
| I-34 | D | D |
| I-35 | B | C |
| I-36 | D | D |
| I-37 | D | D |
| I-38 | D | D |
| I-39 | A | B |
| I-40 | A | B |
| I-41 | A | A |
| I-42 | C | D |
| I-43 | D | D |
| I-44 | C | D |
| I-45 | A | B |
| I-46 | C | D |
| I-47 | A | C |
| I-48 | C | D |
| I-49 | D | D |
| I-50 | B | C |
| I-51 | C | D |
| I-52 | D | D |
| I-53 | B | C |
| I-54 | B | C |
| I-55 | C | D |
| I-56 | C | D |
| I-57 | D | D |
| I-58 | C | D |
| I-59 | B | C |
| I-60 | C | D |
| I-61 | B | C |
| I-62 | C | D |
| I-63 | C | D |
| I-64 | B | C |
| I-65 | B | C |
| I-66 | C | D |
| I-67 | D | D |
| I-68 | C | D |
| I-69 | D | D |
| I-70 | D | D |
| I-71 | D | D |
| I-72 | C | D |
| I-73 | C | D |
| I-74 | D | D |
| I-75 | C | C |
| I-78 | D | D |
| I-79 | D | D |
| I-80 | D | D |
| I-81 | D | D |
| I-82 | C | D |
| I-83 | B | C |
| I-84 | B | C |
| I-85 | C | C |
| I-86 | D | D |
| I-87 | D | D |
| I-88 | C | C |
| I-89 | B | C |
| I-90 | D | D |
| I-91 | A | B |
| I-92 | B | C |
| I-93 | D | D |
| I-94 | D | D |
| I-95 | C | D |
| I-96 | B | C |
| I-97 | B | C |
| I-98 | D | D |
| I-107 | A | D |
| I-108 | B | D |
| I-109 | B | D |
| I-110 | B | D |
| I-122 | D | D |
| I-123 | D | D |
| I-131 | A | C |
| I-132 | C | D |
| I-133 | C | D |
| I-134 | B | C |
| I-135 | C | D |
| I-136 | C | D |
| I-137 | D | D |
| I-138 | B | C |
| I-139 | C | D |
| I-140 | D | D |
| I-141 | C | D |
| I-142 | B | C |
| I-143 | C | C |
| I-144 | C | C |
| I-145 | C | D |
| I-146 | B | B |
| I-147 | D | D |
| I-148 | C | C |
| I-149 | C | C |
| I-150 | D | D |
| I-151 | C | D |
| I-152 | D | D |
| I-153 | C | C |
| I-154 | C | D |
| I-155 | B | C |
| I-156 | D | D |
| I-157 | D | D |
| I-158 | D | D |
| I-159 | D | D |
| I-160 | B | C |
| I-161 | C | C |
| I-162 | D | D |
| I-163 | C | C |
| I-164 | B | C |
| I-165 | A | B |
| I-166 | C | C |
| I-167 | B | C |
| I-168 | C | D |
| I-169 | A | B |
| I-170 | C | D |
| I-171 | D | D |
| I-172 | D | D |
| I-173 | C | D |
| I-174 | C | C |
| I-175 | C | D |
| I-176 | D | D |
| I-177 | C | D |
| I-178 | D | D |
| I-179 | C | D |
| I-180 | D | D |
| I-181 | C | D |
| I-182 | C | D |
| I-183 | D | D |
| I-184 | D | D |
| I-185 | C | D |
| I-186 | D | D |
| I-187 | D | D |
| I-188 | D | D |
| I-189 | D | D |
| I-190 | D | D |
| I-191 | D | D |
| I-192 | D | D |
| I-193 | D | D |
| I-194 | D | D |
| I-195 | D | D |
| I-197 | D | D |
| I-198 | C | D |
| I-199 | D | D |
| I-200 | D | D |
| I-201 | C | C |
| I-202 | B | C |
| I-203 | C | C |
| I-204 | C | D |
| I-205 | D | D |
| I-206 | D | D |
| I-207 | D | D |
| I-208 | D | D |
| I-209 | D | D |
| I-210 | D | D |
| I-211 | C | D |
| I-212 | C | D |
| I-213 | D | D |
| I-214 | D | D |
| I-215 | D | D |
| I-216 | D | D |
| I-217 | D | D |
| I-218 | D | D |
| I-219 | D | D |
| I-220 | D | D |
| I-221 | D | D |
| I-222 | D | D |
| I-223 | D | D |
| I-224 | D | D |
| I-225 | D | D |
| I-226 | D | D |
| I-227 | D | D |
| I-228 | D | D |
| I-229 | D | D |
| I-230 | D | D |
| I-231 | D | D |
| I-232 | D | D |
| I-233 | D | D |
| I-234 | C | D |
| I-235 | D | D |
| I-236 | D | D |
| I-237 | B | C |
| I-238 | B | D |
| I-239 | C | D |
| I-240 | D | D |
| I-241 | D | D |
| I-242 | C | D |
| I-243 | D | D |
| I-244 | D | D |
| I-245 | D | D |
| I-246 | D | D |
| I-247 | C | D |
| I-248 | C | D |
| I-249 | C | D |
| I-250 | D | D |
| I-251 | D | D |
| I-252 | D | D |
| I-253 | D | D |
| I-254 | D | D |
| I-255 | D | D |
| I-256 | D | D |
| I-257 | D | D |
| I-258 | D | D |
| I-259 | C | D |
| I-260 | D | D |
| I-261 | D | D |
| I-262 | D | D |
| I-263 | D | D |
| I-264 | D | D |
| I-265 | D | D |
| I-266 | D | D |
| I-267 | D | D |
| I-268 | D | D |
| I-269 | C | D |
| I-270 | D | D |
| I-271 | D | D |
| I-272 | D | D |
| I-273 | D | D |
| I-274 | D | D |
| I-275 | C | D |
| I-276 | C | D |
| I-277 | D | D |
| I-278 | D | D |
| I-279 | D | D |
| I-280 | D | D |
| I-281 | C | C |
| I-282 | D | D |
| I-283 | D | D |
| I-284 | D | D |
| I-285 | D | D |
| I-286 | D | D |
| I-287 | D | D |
| I-288 | D | D |
| I-289 | D | D |
| I-290 | D | D |
| I-291 | D | D |
| I-292 | D | D |
| I-293 | C | D |
| I-294 | D | D |
| I-295 | D | D |
| I-296 | D | D |
| I-297 | C | D |
| I-298 | D | D |
| I-299 | D | D |
| I-300 | D | D |
| I-301 | D | D |
| I-302 | D | D |
| I-303 | D | D |
| I-304 | D | D |
| I-305 | D | D |
| I-306 | D | D |
| I-307 | D | D |
| I-308 | D | D |
| I-309 | C | D |
| I-310 | D | D |
| I-311 | C | D |
| I-312 | D | D |
| I-313 | D | D |
| I-314 | D | D |
| I-315 | C | D |
| I-316 | D | D |
| I-317 | D | D |
| I-318 | D | D |
| I-319 | D | D |
| I-320 | D | D |
| I-321 | D | D |
| I-322 | D | D |
| I-323 | D | D |
| I-324 | D | D |
| I-325 | D | D |
| I-326 | D | D |
| I-327 | D | D |
| I-328 | D | D |
| I-329 | D | D |
| I-330 | D | D |
| I-331 | D | D |
| I-332 | D | D |
| I-333 | D | D |
| I-334 | D | D |
| I-335 | D | D |
| I-336 | D | D |
| I-337 | D | D |
| I-338 | D | D |
| I-339 | D | D |
| I-340 | D | D |
| I-341 | D | D |
| I-342 | D | D |
| I-343 | D | D |
| I-344 | D | D |
| I-345 | D | D |
| I-346 | D | D |
| I-347 | D | D |
| I-348 | D | D |
| I-349 | D | D |
| I-350 | D | D |
| I-351 | D | D |
| I-352 | D | D |
| I-353 | D | D |
| I-354 | D | D |
| I-355 | D | D |
| I-356 | D | D |
| I-357 | D | D |
| I-358 | D | D |
| I-359 | D | D |
| I-360 | D | D |
| I-361 | D | D |
| I-362 | D | D |
| I-363 | D | D |
| I-364 | D | D |
| I-365 | D | D |
| I-366 | D | D |
| I-367 | D | D |
| I-368 | D | D |
| I-369 | D | D |
| I-370 | D | D |
| I-371 | D | D |
| I-372 | D | D |
| I-373 | D | D |
| I-374 | D | D |
| I-375 | D | D |
| I-376 | D | D |
| I-377 | D | D |
| I-378 | D | D |
| I-379 | D | D |
| I-380 | D | D |
| I-381 | D | D |
| I-382 | D | D |
| I-383 | D | D |
| I-384 | D | D |
| I-385 | D | D |
| I-386 | D | D |
| I-387 | D | D |
| I-388 | D | D |
| I-389 | D | D |
| I-390 | D | D |
| I-391 | D | D |
| I-392 | D | D |
| I-393 | D | D |
| I-394 | D | D |
| I-395 | D | D |
| I-396 | D | D |
| I-401 | D | D |
| I-404 | D | D |
| I-406 | D | D |
| I-408 | D | D |
| I-409 | D | D |
| I-419 | D | D |
| I-420 | D | D |
| I-422 | D | D |
| I-424 | D | D |
| I-425 | D | D |
| I-426 | D | D |
| I-427 | C | D |
| I-428 | D | D |
| I-429 | D | D |
| I-469 | C | B |
| I-470 | C | B |
| I-473 | B | A |
| I-474 | A | A |
| I-475 | B | A |
| I-476 | B | A |
| I-477 | A | A |

### Example 8. Degradation in HEK293 Cells

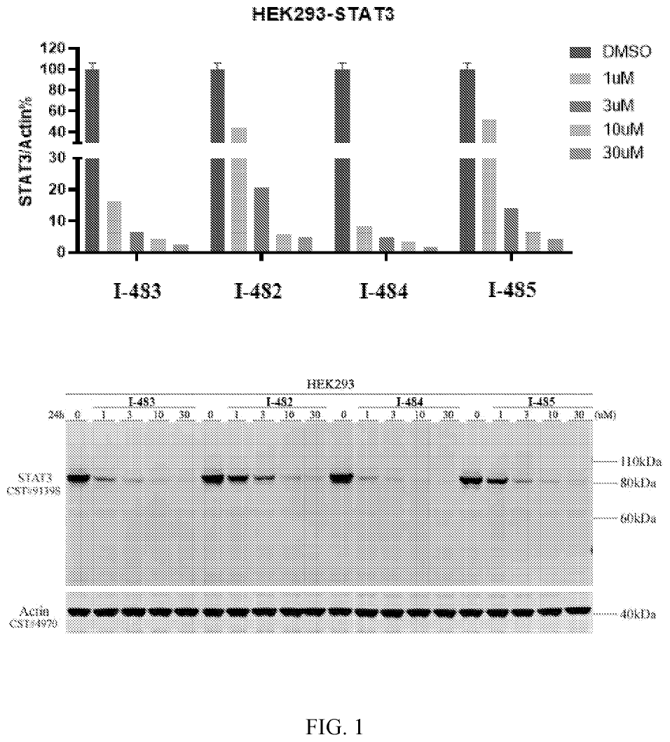

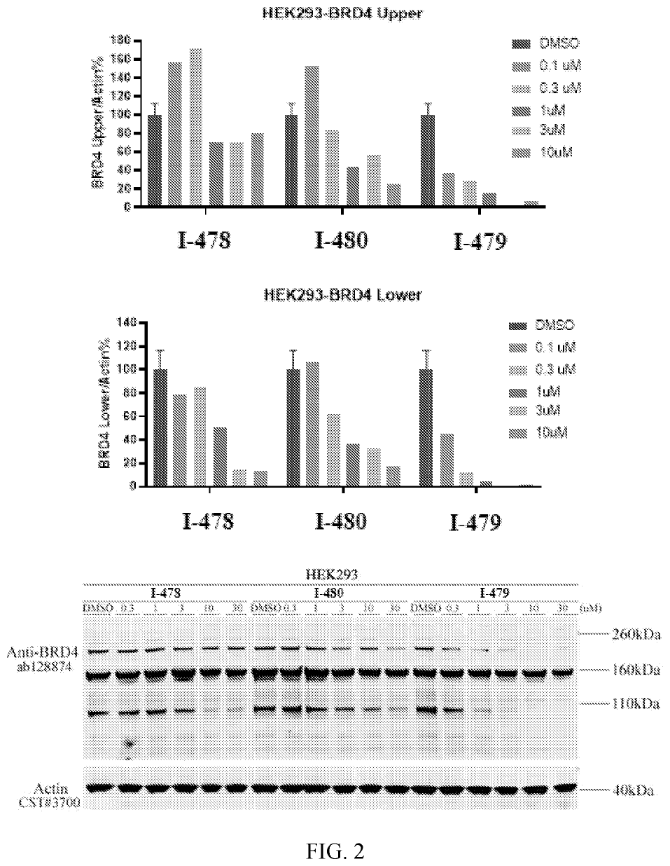

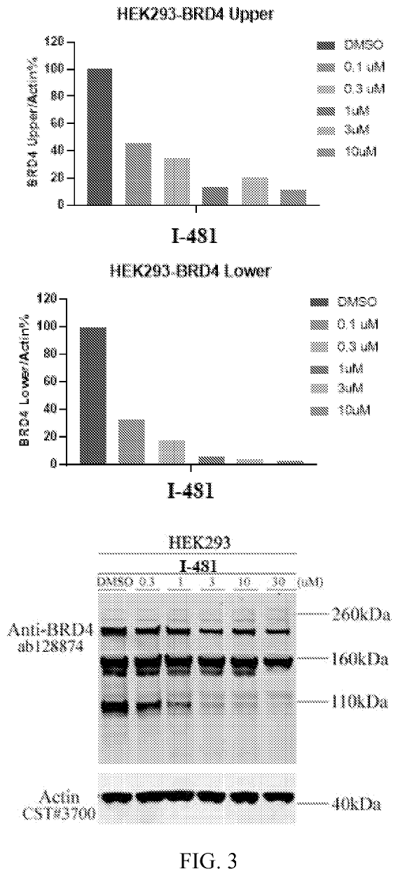

On day 1, 6.25×10⁵ HEK293 cells were seeded per well into a 12-well plate. On day 2, compounds were added to the cells at the concentrations shown in FIGS. 1-4. After 24, cells were lysed in wells with 80 ul of pre-chilled RIPA Lysis buffer (Beyotime, P0013B) with protease/phosphatase inhibitor (Roche 4693116001/Roche 04906837001) for 20 min at 4 degree on a rocker, then spun down at 20,000g at 4 degree for 10 min and the BCA assay was run. 39 ul of supernatant was transferred to a fresh EP tube containing 15 ul of 4X LDS loading buffer and 6 ul 10X reducing agent to make the loading samples. The samples were then heated to 70° C. for 10 minutes, cooled to RT, and stored under−20° C. Samples were loaded with equal amount of protein onto 4-12% Bis -Tris SDS-PAGE 26 wells gel (Novex, WG1403BOX) and the gel was run for 1.5 hours at 120 V. Electrotransfer to NC membrane was completed using wet-transfer method with 250 mA for 80 min and the membrane was blocked in 5% BSA (Solarbio,A8020) with 1X TBST for 1 hour. The samples were incubated with primary antibody prepared in blocking buffer (LI-COR, 927-60001) at 4° C. overnight and the membrane was washed three times with 1X TBST, 5 minutes each. Incubate with secondary antibody for 1 hour at RT (anti-rabbit IgG (Licor,926-32211) 1:5000; anti-mouse IgG (LI-COR, 926-68070)1:5000). The membrance was then washed three times with 1X TBST, 5 minutes each and once with RO H₂O, then read on LiCOR.

STAT3 and BRD4 degradation results are shown in FIGS. 1-4.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of formula I-a: 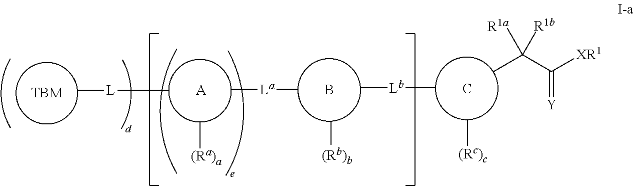
or a pharmaceutically acceptable salt thereof, wherein:
R¹, R^{1a} and R^{1b} are each independently hydrogen or optionally substituted C₁₋₆ aliphatic;
each R^{a}, R^{b}, and R^{o} are each independently hydrogen, R^{A}, halogen, —CN, —NO₂, —OR, —SR, —NR₂, —S(O)₂R, —S(O)₂NR₂, —S(O)R, —S(O)(NR)R—P(O)(OR)₂, —P(O)(NR₂)₂, —CFR₂, —CRF₂, —CF₃, —CR₂ (OR), —CR₂ (NR₂), —C(O)R, —C(O) OR, or —C(O)NR₂;
each R^{A} is independently an optionally substituted group selected from C₁₋₁₀ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are optionally taken together with their intervening atom to form an optionally substituted 4-11 membered saturated or partially unsaturated carbocyclic or heterocyclic monocyclic, bicyclic, bridged bicyclic, spirocyclic, or heteroaryl ring having 0-3 heteroatoms, in addition to the atom to which they are attached, independently selected from nitrogen, oxygen, and sulfur;
Ring A is bivalent ring selected from phenylenyl, naphthylenyl, a 4-10 membered saturated or partially unsaturated monocyclic or bicyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur;
Ring B is bivalent ring selected from phenylenyl, a 3-10 membered saturated or partially unsaturated monocyclic, bicyclic, bridged bicyclic, or spirocyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur;
Ring C is bivalent ring selected from phenylenyl, a 4-10 membered saturated or partially unsaturated monocyclic or bicyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur;
each of L^{a} and L^{b} is independently a covalent bond or a C₁₋₃ bivalent straight or branched saturated or unsaturated hydrocarbon chain wherein 1-3 methylene units of the chain are independently and optionally replaced with —O—, —C(O)—, —C(S)—, —C(R)₂—, —CH(R)—, —CF(R)—, —C(F)₂—, —N(R)—, —S—, —S (O)₂- or —CR═CR—;
a, b, and c are each independently 0, 1, 2, 3 or 4;
each of e and d is independently 0 or 1;
X is —O—, —N(R)—, or —S—;
Y is O, N (R), or S;
L is a covalent bond or a bivalent, saturated or unsaturated, straight or branched C₁₋₅₀ hydrocarbon chain, wherein 0-6 methylene units of L are independently replaced by —Cy—, —CRF—, —CF₂—, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N (R)—, —N(R)C(O)—, —C(O)N(R)—, —OC(O)N(R)—, —N (R)C(O)O—, 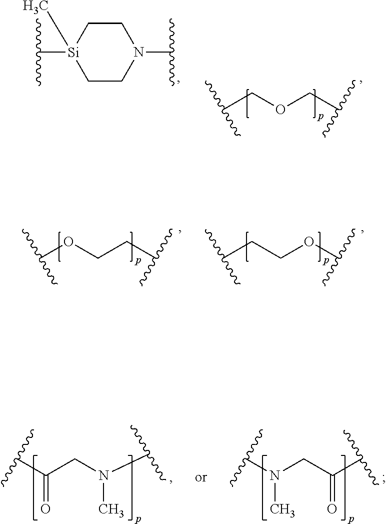
each —Cy— is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
each p is independently 0, 1, 2, 3, 4, 5, 6, 7, 8, 9, or 10; and
TBM is a target binding moiety.

2. The compound of claim 1, wherein said compound is any one of the following formulae: 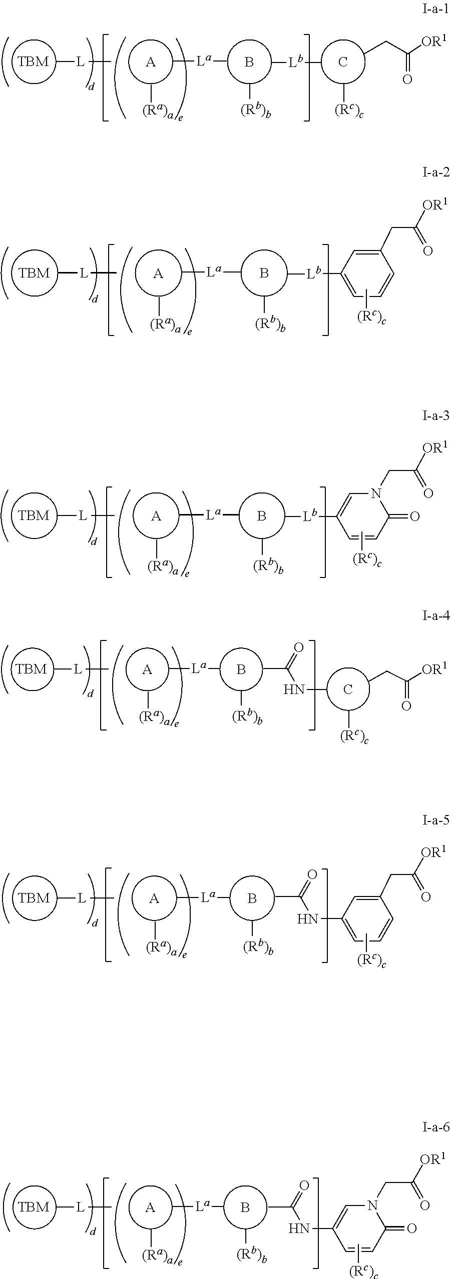
or a pharmaceutically acceptable salt thereof.

3. The compound of any one of claims 1-2, wherein R¹ is hydrogen, methyl, or ethyl.

4. The compound of any one of claims 1-3, wherein Ring A is bivalent ring selected from phenylenyl, naphthylenyl, or a 5-10 membered monocyclic or bicyclic heteroarylenyl having 1-4 heteroatoms independently selected from nitrogen, oxygen, and sulfur.

5. The compound of any one of claims 1-4, wherein each Ring B is bivalent ring selected from phenylenyl, a 5-6 membered saturated or partially unsaturated monocyclic carbocyclylenyl or heterocyclylenyl having 1-3 heteroatoms independently selected from nitrogen, oxygen, and sulfur, or a 5-10 membered monocyclic or bicyclic heteroarylenyl having 1-4 heteroatoms independently selected from nitrogen, oxygen, and sulfur.

6. The compound of any one of claims 1-5, wherein L is a covalent bond or a bivalent, saturated or unsaturated, straight or branched C₁₋₂₀ hydrocarbon chain, wherein 0-6 methylene units of L are independently replaced by —CRF—, —CF₂—, —Cy—, —O—, —N(R)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, and —N(R)C(O)O—.

7. The compound of any one of claims 1-6, wherein TBM is a target binding moiety that binds to a target protein selected from the group listed in paragraph [00274].

8. The compound of any one of claims 1-7, wherein TBM is

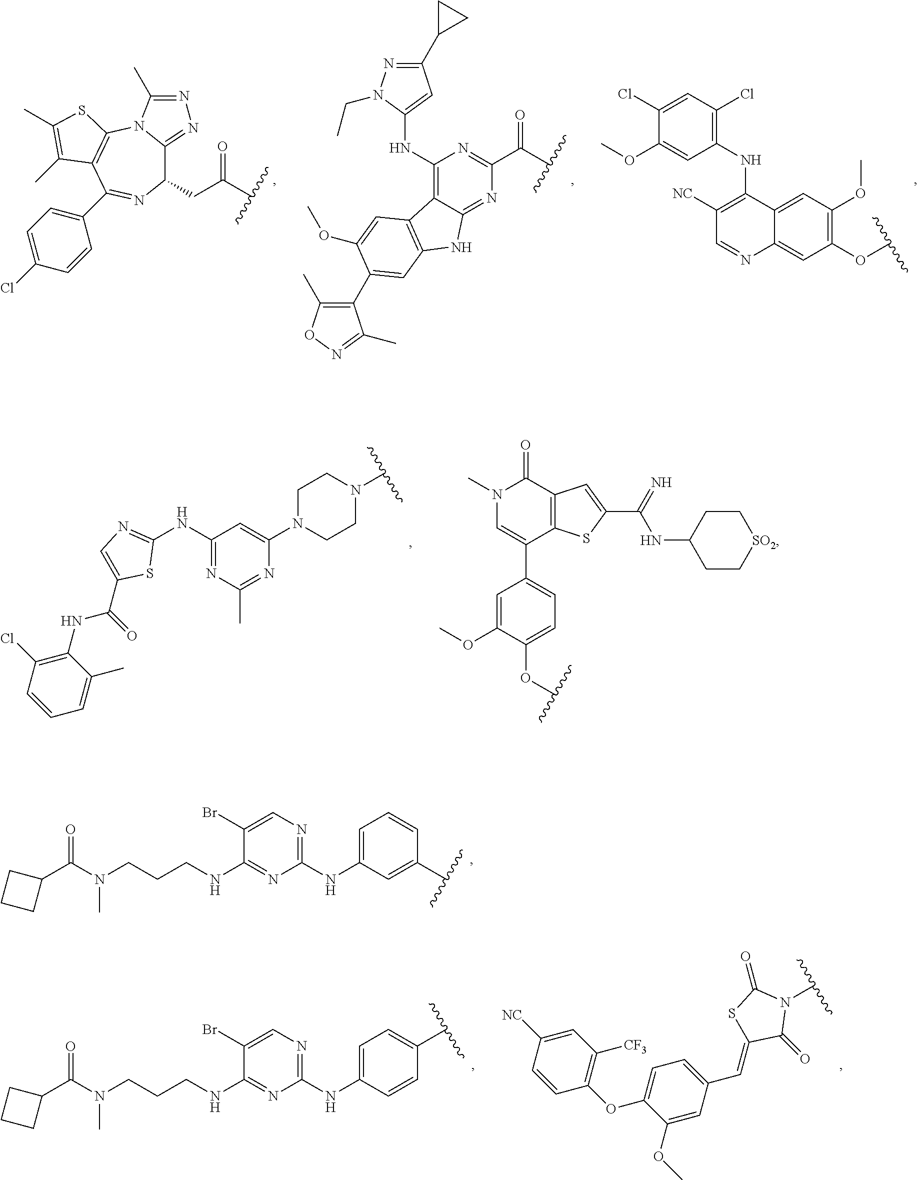

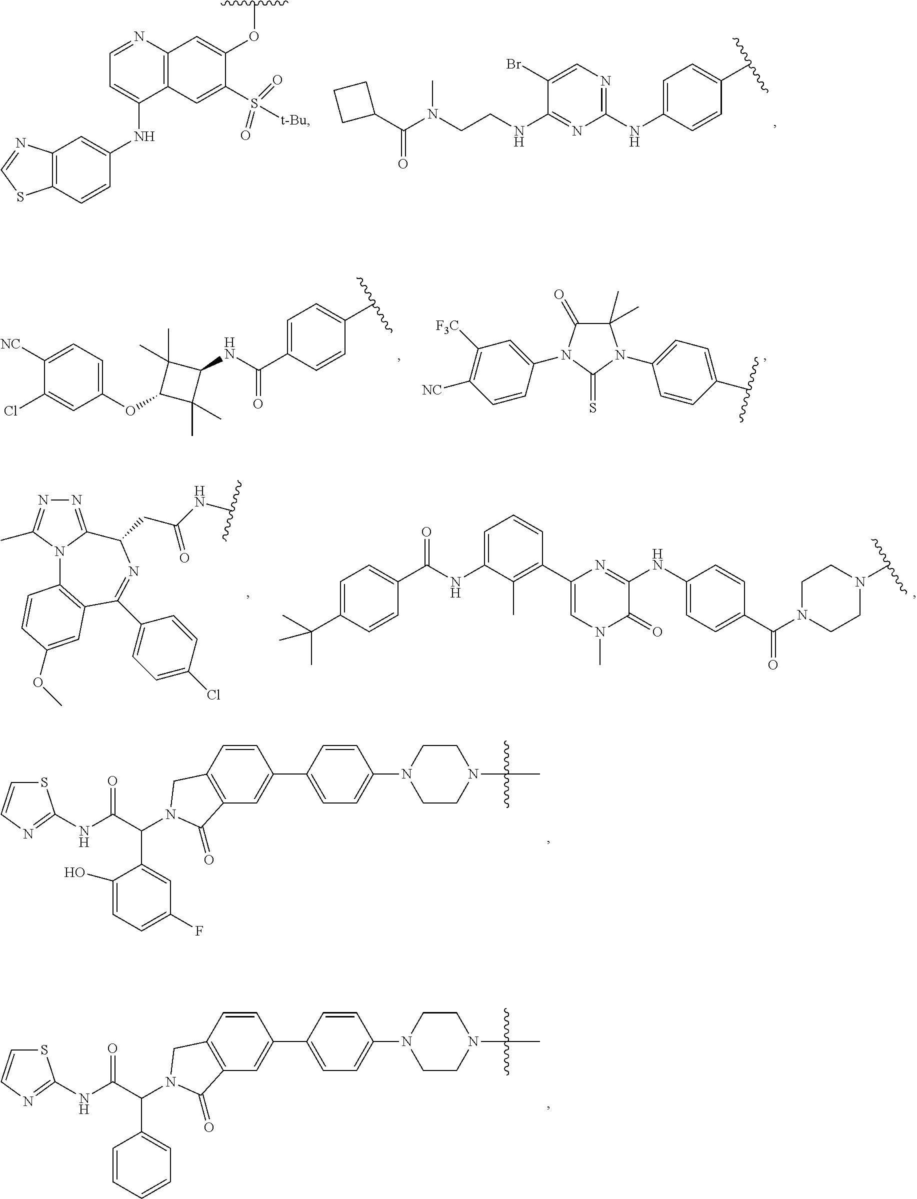

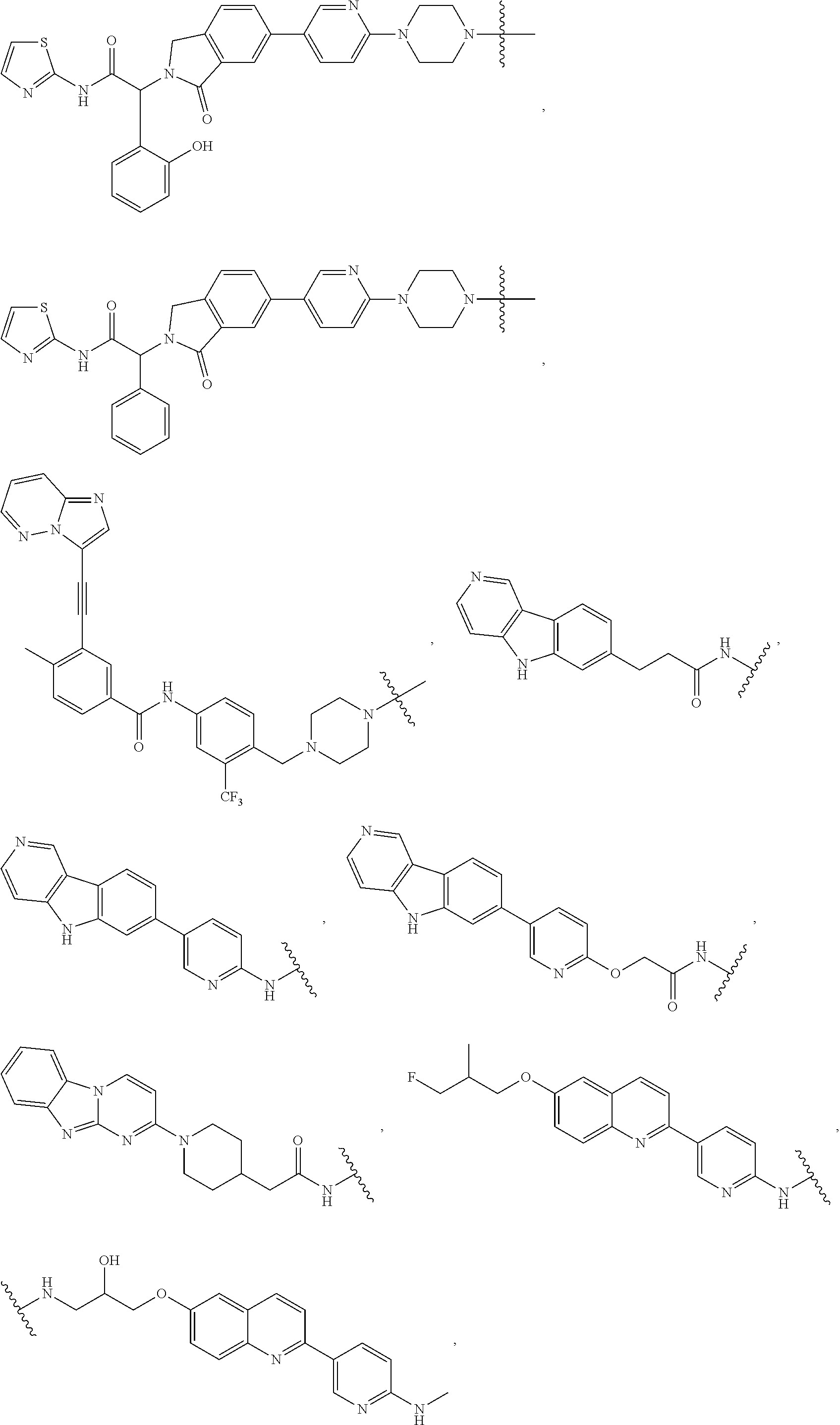

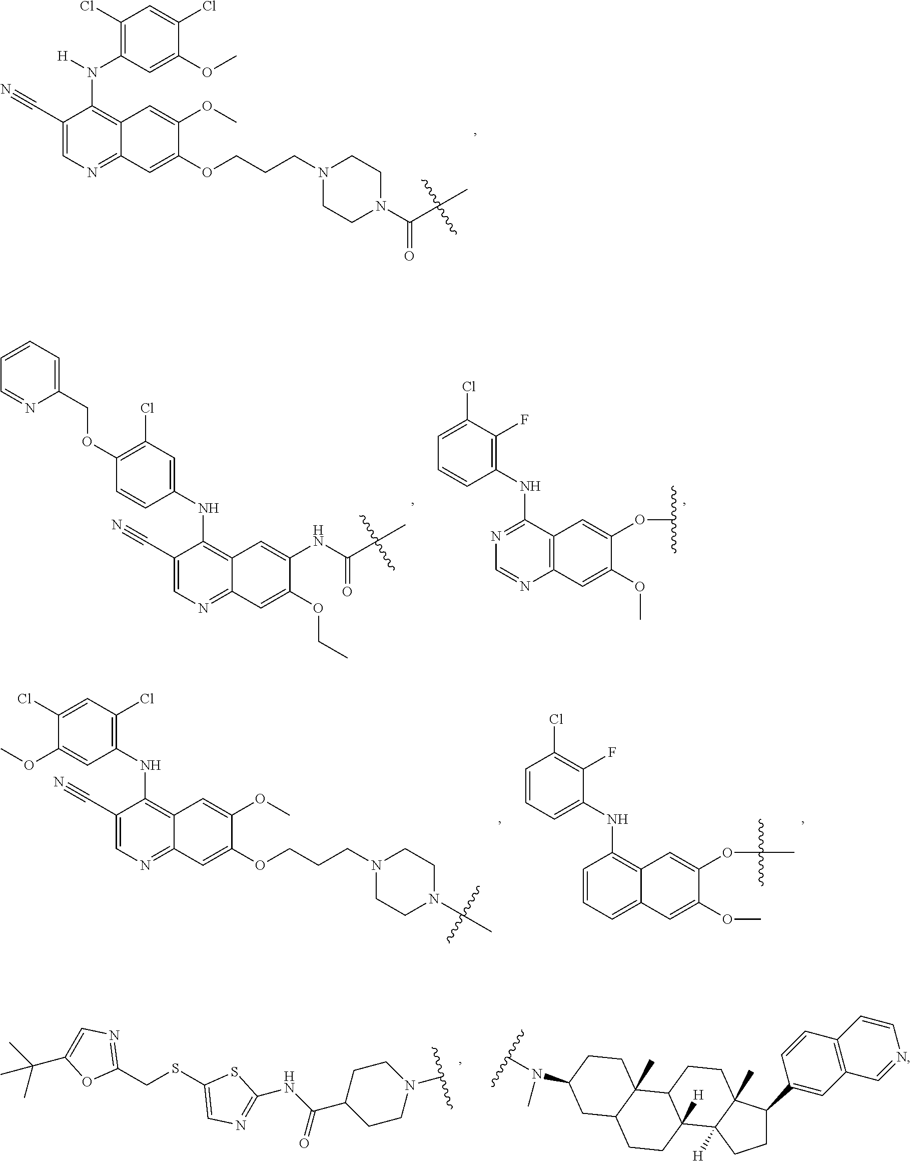

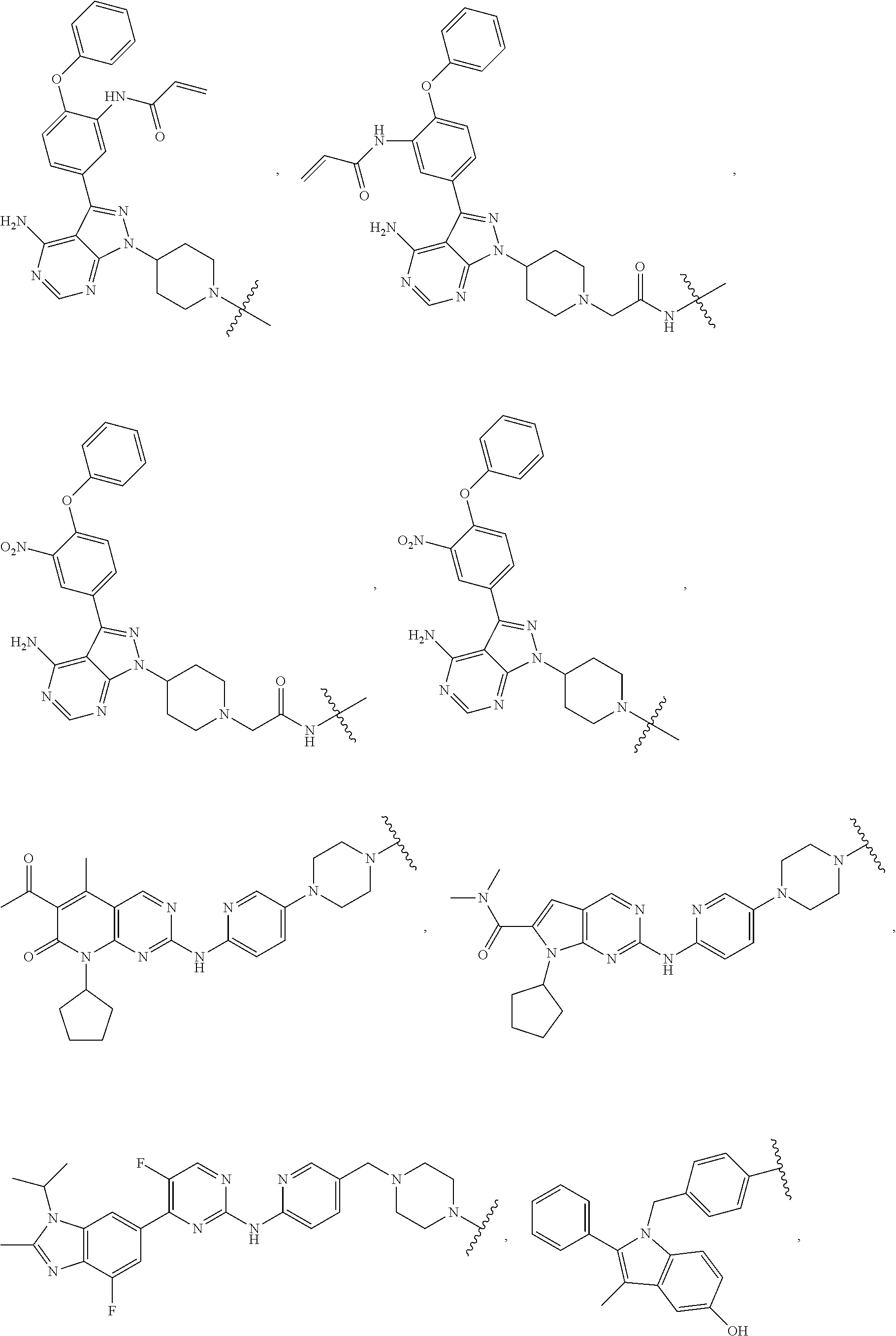

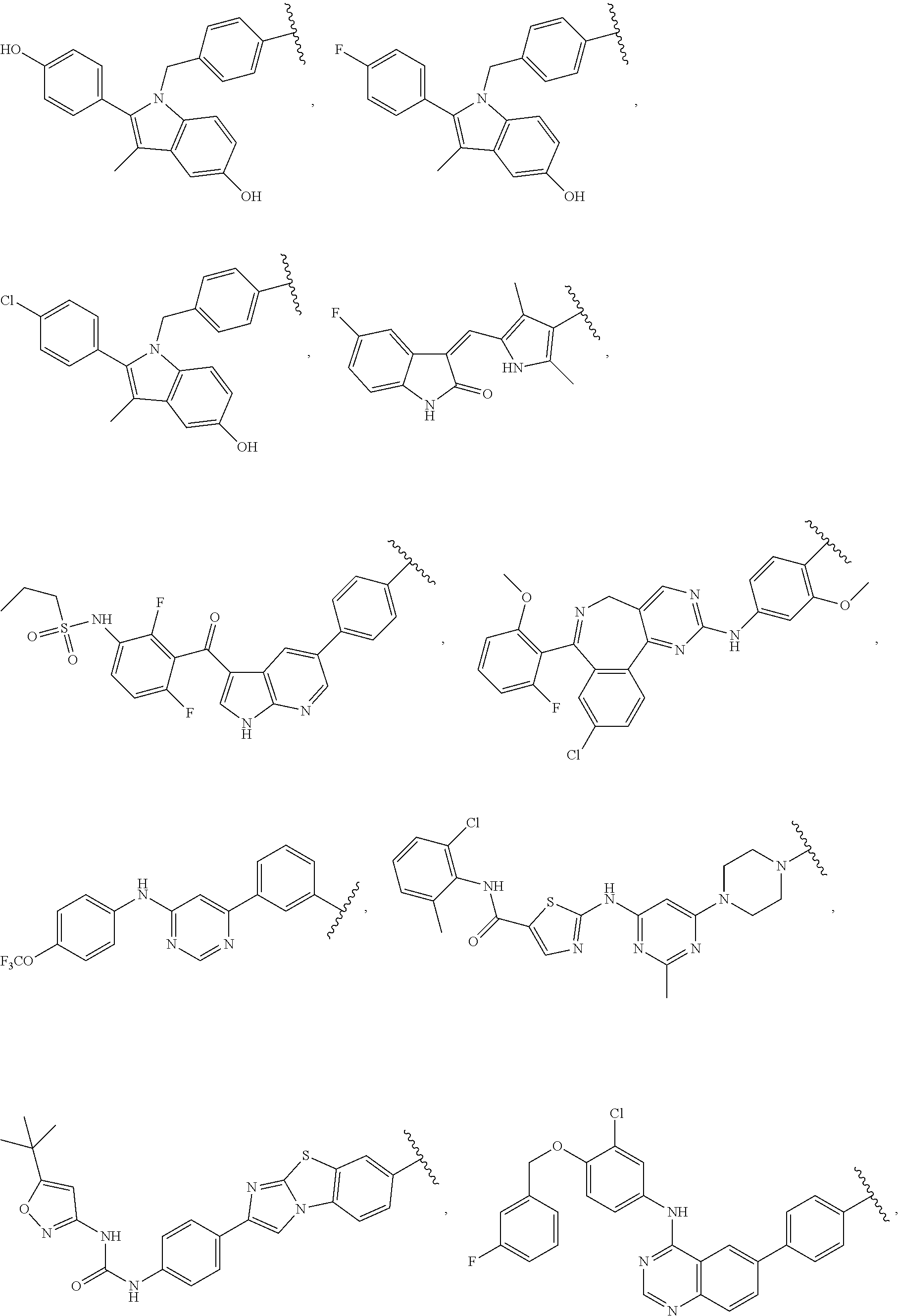

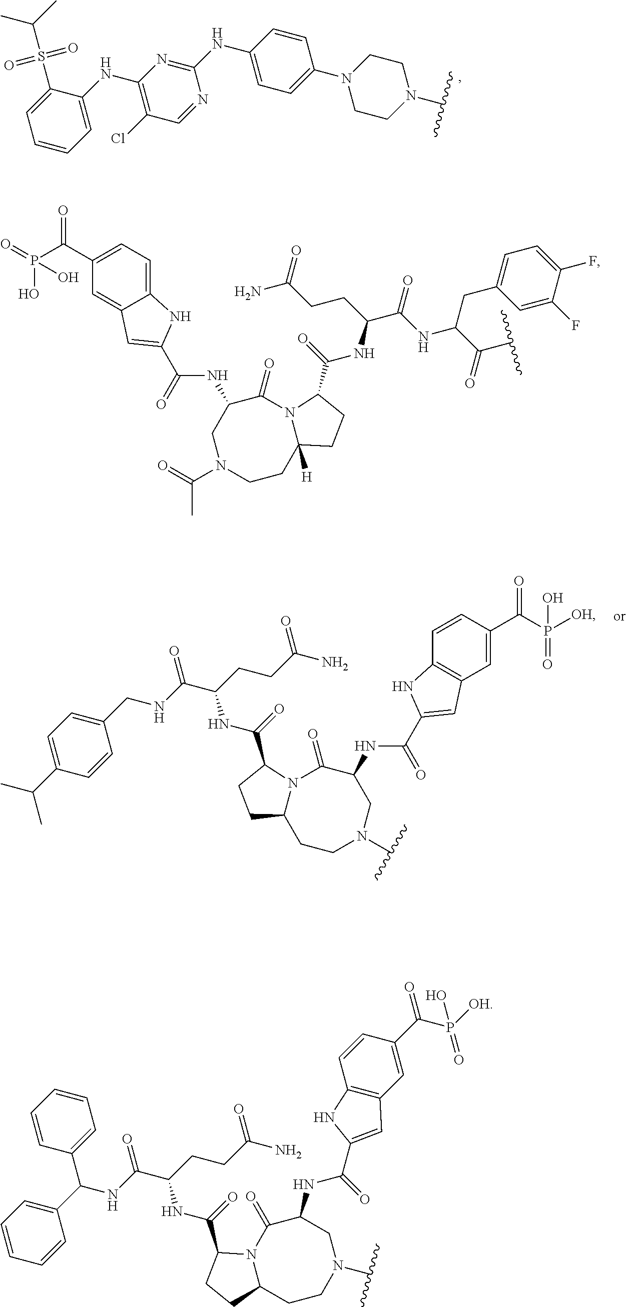 [SMILES: *N1CCN(c2ccc(Nc3ncc(Cl)c(Nc4ccccc4S(=O)(=O)C(C)C)n3)cc2)CC1 ; *C(=O)C(Cc1ccc(F)c(F)c1)NC(=O)[C@H](CCC(N)=O)NC(=O)[C@@H]1CC[C@@H]2CCN(C(C)=O)C[C@H](NC(=O)c3cc4cc(C(=O)P(=O)(O)O)ccc4[nH]3)C(=O)N21 ; *N1CC[C@H]2CC[C@@H](C(=O)N[C@@H](CCC(N)=O)C(=O)NCc3ccc(C(C)C)cc3)N2C(=O)[C@@H](NC(=O)c2cc3cc(C(=O)P(=O)(O)O)ccc3[nH]2)C1 ; *N1CC[C@H]2CC[C@@H](C(=O)N[C@@H](CCC(N)=O)C(=O)NC(c3ccccc3)c3ccccc3)N2C(=O)[C@@H](NC(=O)c2cc3cc(C(=O)P(=O)(O)O)ccc3[nH]2)C1]

9. The compound of any one of claims 1-8, wherein said compound is selected from those depicted in Table 1A or Table 1B of the specification, or a pharmaceutically acceptable salt thereof.

10. A pharmaceutical composition comprising a compound according to any one of the preceding claims, or a pharmaceutically acceptable salt thereof, and a pharmaceutically acceptable carrier, adjuvant, or vehicle.

11. A method of degrading a target protein in a biological sample comprising contacting the sample with the compound of any one of claims 1-9, or a pharmaceutically acceptable salt thereof, wherein the target protein is selected from group listed in paragraph [00274].

12. A method of treating a target protein-mediated disorder, disease, or condition in a patient comprising administering to said patient the compound of any one of claims 1-8 or a pharmaceutical composition thereof.

13. The method of claim 12, wherein the disorder is selected from an autoimmune disorder, an inflammatory disorder, a proliferative disorder, an endocrine disorder, a neurological disorder, or a disorder associated with transplantation.

14. The method of claim 13, wherein the disorder is a proliferative disorder.

15. The method of claim 14, wherein the proliferative disorder is a cancer.

16. The method of claim 15, wherein the cancer is squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and non-Hodgkin's lymphoma; benign and malignant melanomas; myeloproliferative diseases; multiple myeloma, sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor or teratocarcinomas, T-lineage acute lymphoblastic leukemia (T-ALL), T-lineage lymphoblastic lymphoma (T-LL), peripheral T-cell lymphoma, Adult T-cell leukemia, Pre-B ALL, Pre-B lymphomas, large B-cell lymphoma, Burkitts lymphoma, B-cell ALL, Philadelphia chromosome positive ALL and Philadelphia chromosome positive CML.
